(19) United States
(12) Patent Application Publication
(10) Pub. No.: US 2018/0305348 A1
(43) Pub. Date: Oct. 25, 2018

(54) INHIBITORS OF BRUTONS TYROSINE KINASE

(71) Applicant: Pharmacyclics LLC, Sunnyvale, CA (US)

(72) Inventors: Gordana B. Attallah, Fremont, CA (US); Wei Chen, Fremont, CA (US); Zhaozhong J. Jia, San Mateo, CA (US); Alfonso Pozzan, Venice (IT); Luca F. Raveglia, Milan (IT); Riccardo Zanaletti, Verona (IT)

(21) Appl. No.: 15/579,142

(22) PCT Filed: Jun. 2, 2016
(86) PCT No.: PCT/US16/35489

§ 371 Date: Dec. 1, 2017

Related U.S. Application Data
(60) Provisional application No. 62/249,338, filed on Nov. 1, 2015.
(60) Provisional application No. 62/249,340, filed on Nov. 1, 2015.
(60) Provisional application No. 62/249,336, filed on Nov. 1, 2015.
(60) Provisional application No. 62/169,945, filed on Jun. 2, 2015.
(60) Provisional application No. 62/169,941, filed on Jun. 2, 2015.
(60) Provisional application No. 62/169,935, filed on Jun. 2, 2015.

(51) Int. Cl. C07D 417/14; C07D 401/14; C07D 401/04; C07D 403/04; C07D 405/14; C07D 409/14; C07D 413/14; C07D 471/04; C07D 471/08; A61P 35/02; A61P 19/02; A61P 19/10; A61P 29/00; A61P 37/02

(52) U.S. Cl. CPC C07D 417/14; C07D 401/14; C07D 401/04; C07D 403/04; C07D 405/14; C07D 409/14; A61P 37/02; C07D 471/04; C07D 471/08; A61P 35/02; A61P 19/02; A61P 19/10; A61P 29/00; C07D 413/14

(57) ABSTRACT

Disclosed herein are reversible and irreversible inhibitors of Bruton's tyrosine kinase (Btk). Also disclosed are pharmaceutical compositions that include the compounds. Methods of using the Btk inhibitors are described, alone or in combination with other therapeutic agents, for the treatment of autoimmune diseases or conditions, heteroimmune diseases or conditions, cancer, including lymphoma, and inflammatory diseases or conditions.

### CROSS-REFERENCE TO RELATED APPLICATIONS

This application claims the benefit of U.S. Provisional Application No. 62/169,935, filed Jun. 2, 2015; U.S. Provisional Application No. 62/249,336, filed Nov. 1, 2015; U.S. Provisional Application No. 62/169,941, filed Jun. 2, 2015; U.S. Provisional Application No. 62/249,338, filed Nov. 1, 2015; U.S. Provisional Application No. 62/169,945, filed Jun. 2, 2015; and U.S. Provisional Application No. 62/249,340, filed Nov. 1, 2015. Each of said applications is incorporated herein by reference in its entirety.

### FIELD OF THE INVENTION

Described herein are compounds, methods of making such compounds, pharmaceutical compositions and medicaments containing such compounds, and methods of using such compounds and compositions to inhibit the activity of tyrosine kinases.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival (Kurosaki, Curr Op Imm, 2000, 276-281; Schaeffer and Schwartzberg, Curr Op Imm 2000, 282-288). In addition, Btk plays a role in a number of other hematopoetic cell signaling pathways, e.g., Toll like receptor (TLR) and cytokine receptor-mediated TNF-α production in macrophages, IgE receptor (FcepsilonRI) signaling in Mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

### SUMMARY OF THE INVENTION

Described herein are inhibitors of Bruton's tyrosine kinase (Btk). Also described herein are irreversible inhibitors of Btk. Also described herein are reversible inhibitors of Btk. Further described are irreversible inhibitors of Btk that form a covalent bond with a cysteine residue on Btk. Further described herein are irreversible inhibitors of other tyrosine kinases, wherein the other tyrosine kinases share homology with Btk by having a cysteine residue (including a Cys 481 residue) that can form a covalent bond with the irreversible inhibitor (such tyrosine kinases, are referred herein as “Btk tyrosine kinase cysteine homologs”).

Also described herein are methods for synthesizing such reversible or irreversible inhibitors, methods for using such reversible or irreversible inhibitors in the treatment of diseases (including diseases wherein irreversible inhibition of Btk provides therapeutic benefit to a patient having the disease). Further described are pharmaceutical formulations that include a reversible or irreversible inhibitor of Btk.

In one aspect, provided herein are compounds of Formula (I) and pharmaceutically acceptable solvates, pharmaceutically acceptable salts, and/or pharmaceutically acceptable prodrugs thereof:

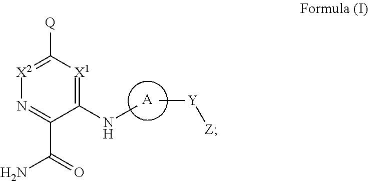

wherein:
Q is

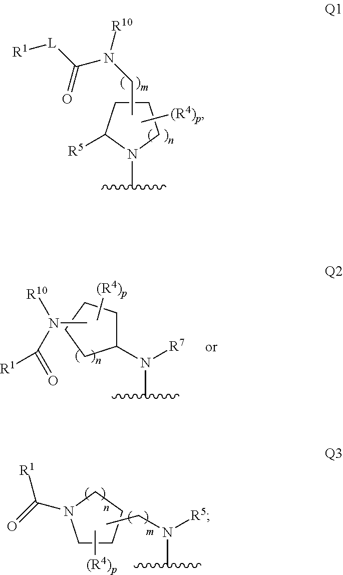

ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a single bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

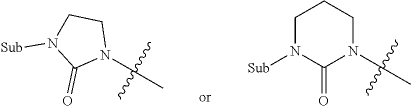

(wherein Sub represents H or a substituent); when m is 1, R¹ may also be substituted or unsubstituted C₁-C₄alkyl;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or two R⁴ form a C₁-C₄alkylene;
R⁵ is H, halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or R⁵ together with one R⁴ form a C₁-C₄alkylene;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3:
or a pharmaceutically acceptable solvate, pharmaceutically acceptable salt, and/or pharmaceutically acceptable prodrug thereof;
provided that:
(1) when Q is Q1, m is 0, R¹ is substituted or unsubstituted C₂-C₄alkenyl, and X¹ is N, then X² is other than C(Et);
(2) when Q is Q1, and m is 0, then -A-Y—Z is other than

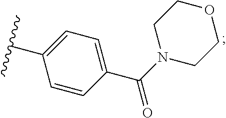 [SMILES: *c1ccc(C(=O)N2CCOCC2)cc1]

(3) when Q is Q1, and m is 0, then R¹ is other than

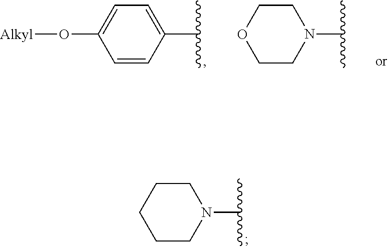

(4) when Q is Q2, R¹ is substituted or unsubstituted C₂-C₄alkenyl, A is substituted or unsubstituted phenyl, R¹ is H, the group

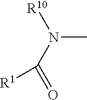

and the group

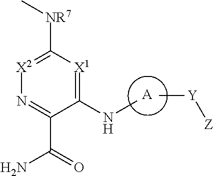

are attached to the same carbon atom or attached to carbon atoms that are adjacent to each other, and X¹ is N, then X² is other than CH or C(Et)
(5) when Q is Q3, and m is 0, then -A-Y—Z is other than

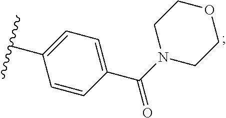 [SMILES: *c1ccc(C(=O)N2CCOCC2)cc1]

(6) when Q is Q3, m is 0, A is quinolinyl, X¹ is N and X² is CH, then R¹ is other than Me;
(7) when Q is Q3, and X¹ is N, then X² is CH or N; and
(8) the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-yl)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperidin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrahydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrahydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide;
(R)-5-(3-(3-(tetrahydro-2H-pyran-4-yl)ureido)piperidin-1-yl)-3-(tetrahydro-2H-pyran-4-ylamino)pyrazine-2-carboxamide;
5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide;
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide; or
(R)-3-(5-carbamoyl-6-(3-methylisothiazol-5-ylamino)pyrazin-2-ylamino)-N,N-dimethylazepane-1-carboxamide.

In another aspect, provided herein is a compound of Formula (A-I) having the structure:

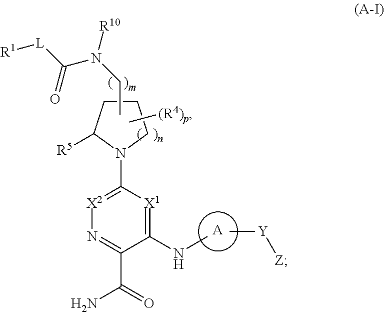

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a single bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

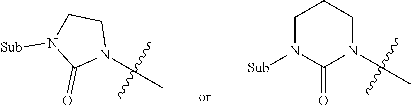

(wherein Sub represents H or a substituent); when m is 1, R¹ may also be substituted or unsubstituted C₁-C₄alkyl;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or two R⁴ form a C₁-C₄alkylene; R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or R⁵ together with one R⁴ form a C₁-C₄alkylene;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3;
or a pharmaceutically acceptable solvate, pharmaceutically acceptable salt, and/or pharmaceutically acceptable prodrug thereof;
provided that:
(1) when in is 0, R¹ is substituted or unsubstituted C₂-C₄alkenyl, and X¹ is N, then X² is other than C(Et);
(2) when in is 0, then -A-Y—Z is other than

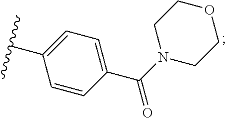 [SMILES: *c1ccc(C(=O)N2CCOCC2)cc1]

(3) when m is 0, then R¹ is other than

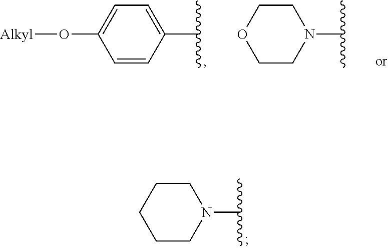

and
(4) the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-yl)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperidin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrahydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrahydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
5-((2R,3R)-3-(3,3-dimethylureido)-2-methylpiperidin-1-yl)-3-(3-methylisothiazol-5-ylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide; and
(R)-5-(3-(3-(tetrahydro-2H-pyran-4-yl)ureido)piperidin-1-yl)-3-(tetrahydro-2H-pyran-4-ylamino)pyrazine-2-carboxamide;
   and a pharmaceutically acceptable solvate, pharmaceutically acceptable salt, and/or pharmaceutically acceptable prodrug thereof.

In one aspect, the compound is a compound of Formula (A-I) wherein A, L, X¹, X², Y, Z, R¹⁰, m, n and p are as defined above;

each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; and
R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂.

In another aspect, provided herein is a compound of Formula (B-I) having the structure:

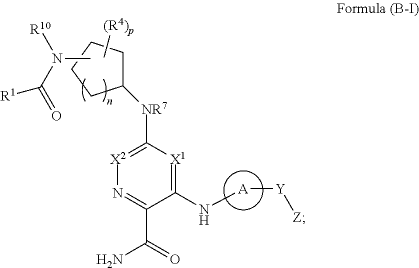

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R¹⁰ together with the —C(O)—N— between them form a substituted or unsubstituted C₁-C₁₂heteroaryl or substituted or unsubstituted C₂-C₇heterocycloalkyl optionally fused with a substituted or unsubstituted phenyl ring;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is substituted or unsubstituted C₁—C(alkyl, substituted or unsubstituted C₂-C₄alkenyl, substituted or unsubstituted C₂-C₄alkynyl, substituted or unsubstituted C₆-C₇cycloalkyl, substituted or unsubstituted C₂-C₇heterocycloalkyl, substituted or unsubstituted C₆-C₁₂aryl, or substituted or unsubstituted C₁-C₁₂heteroaryl;
R⁷ is H, substituted or unsubstituted C₁-C₄alkyl or C(O)—(C₂-C₄alkenyl);
R¹⁰ and R¹¹ are independently H, or substituted or unsubstituted C₁-C₄alkyl; or R¹⁰ and R¹¹ connect to form a C₁-C₄alkylene;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when R¹ is substituted or unsubstituted C₂-C₄alkenyl, A is substituted or unsubstituted phenyl, R⁷ is H, the group

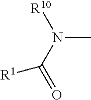

and the group

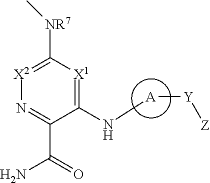

are attached to the same carbon atom or attached to carbon atoms that are adjacent to each other, and X¹ is N, then X² is other than CH or C(Et); and
(2) the compound is other than 5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide.

In one aspect, provided herein is a compound of Formula (A-II) having the structure:

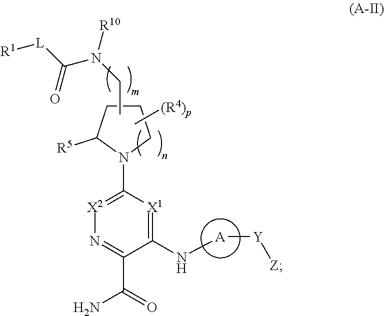

wherein:
ring A is substituted or unsubstituted C₆-C₁₂aryl, or substituted or unsubstituted C₁-C₁₂heteroaryl;
X¹ and X² are both N or are both CH; or X¹ is N and X² is CH;
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a single bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

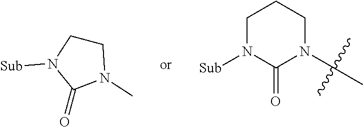

(wherein Sub represents H or a substituent); when m is 1, R¹ may also be substituted or unsubstituted C₁-C₄alkyl;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or two R⁴ form a C₁-C₄alkylene;
R⁵ is H, halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or R⁵ together with one R⁴ form a C₁-C₄alkylene;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3;
or a pharmaceutically acceptable solvate, pharmaceutically acceptable salt, and/or pharmaceutically acceptable prodrug thereof;
provided that
(1) when m is 0, then -A-Y—Z is other than

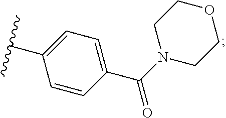 [SMILES: *c1ccc(C(=O)N2CCOCC2)cc1]

(2) when m is 0, then R¹ is other than

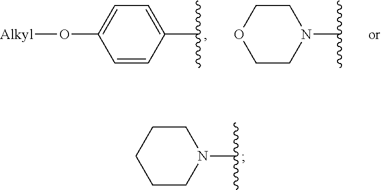

and
(3) the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-yl)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperdin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrabydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrabydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide; and
(R)-5-(3-(3-(tetrahydro-2H-pyran-4-yl)ureido)piperidin-1-yl)-3-(tetrahydro-2H-pyran-4-ylamino)pyrazine-2-carboxamide;
   or a pharmaceutically acceptable solvate, pharmaceutically acceptable salt, and/or pharmaceutically acceptable prodrug thereof.

In one aspect, the compound is a compound of Formula (A-II) wherein A, L, X¹, X², Y, Z, R¹⁰, m, n and p are as defined above;

each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; and
R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂.

In one aspect, provided herein is a compound of Formula (A-VI) having the structure:

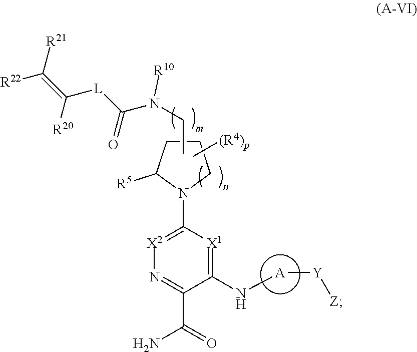

wherein:
wherein A, L, X¹, X², Y, Z, R⁴, R⁵, R¹⁰, m, n and p are as defined herein; and R²⁰, R²¹ and R²² are each independently H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, and/or pharmaceutically acceptable prodrug thereof.

In one aspect, provided herein is a compound of Formula (A-IA), (A-IB), (A-IC), (A-ID) or (A-IE) having the structure:

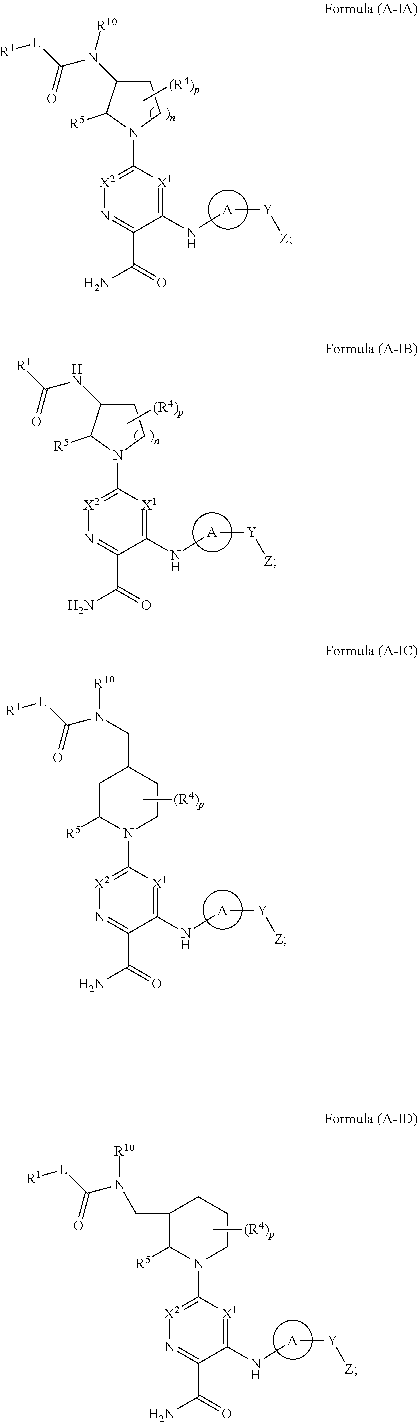

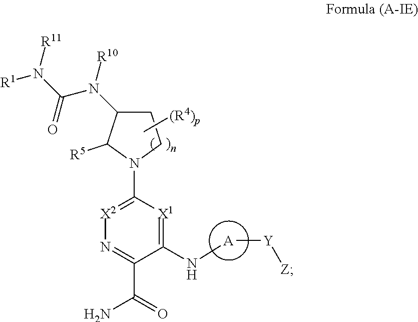

wherein A, L, X¹, X², Y, Z, R¹, R⁴, R⁵, R¹⁰, R¹¹, n and p are as defined herein; or a pharmaceutically acceptable solvate, pharmaceutically acceptable salt, and/or pharmaceutically acceptable prodrug thereof.

In some embodiments, the compound is a compound of Formula (A-I) or (A-II) wherein the group

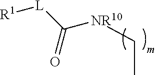

is not attached to a carbon atom adjacent to the nitrogen atom in the ring that it is attached to.

In some embodiments, the compound is a compound of Formula (A-I), (A-II) or (A-IA)-(A-IE) wherein R¹ is substituted or unsubstituted C₂-C₄alkenyl. In some embodiments, the compound is a compound of Formula (A-I), (A-II) or (IA)-(IE) wherein R¹ is substituted or unsubstituted C₂-C₄alkynyl. In some embodiments, the compound is a compound of Formula (A-I), (A-II) or (IA)-(IE) wherein R¹ is substituted or unsubstituted C₆-C₁₂aryl, or substituted or unsubstituted C₁-C₁₂heteroaryl. In some embodiments, the compound is a compound of Formula (A-I), (A-II) or (IA)-(IE) wherein R¹ is substituted or unsubstituted phenyl, pyridyl, pyrimidinyl, indolyl, benzimidazolyl, or benzofuranyl. In some embodiments, the compound is a compound of Formula (A-I), (A-II) or (IA)-(IE) wherein R¹ is substituted or unsubstituted isoindolinyl.

In one aspect, provided herein is a compound of Formula (B-II) having the structure:

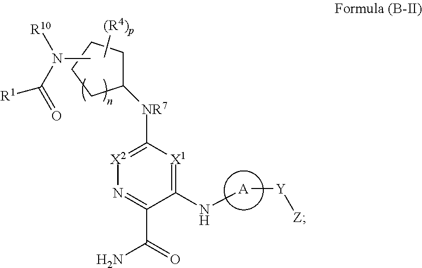

wherein:
ring A is substituted or unsubstituted C₆-C₁₂aryl, or substituted or unsubstituted C₁-C₁₂heteroaryl;
X¹ and X² are both N or are both CH; or X¹ is N and X² is CH;
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is unsubstituted or substituted C₁-C₄alkyl, substituted or unsubstituted C₂-C₄alkenyl, substituted or unsubstituted C₂-C₄alkynyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R^{1′} together with the —C(O)—N— moiety between them form a substituted or unsubstituted C₁-C₁₂heteroaryl or substituted or unsubstituted C₂-C₇heterocycloalkyl optionally fused with a substituted or unsubstituted phenyl ring;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₇heterocycloalkyl, or —N(R³)₂;
R⁵ is unsubstituted or substituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl;
R⁷ is H, or substituted or unsubstituted C₁-C₄alkyl;
R¹⁰ and R¹¹ are independently H, or substituted or unsubstituted C₁-C₄alkyl; or R¹⁰ and R¹¹ connect to form a C₁-C₄alkylene;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that when R¹ is substituted or unsubstituted C₂-C₄alkenyl, A is substituted or unsubstituted phenyl, R⁷ is H, the group

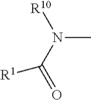

and the group

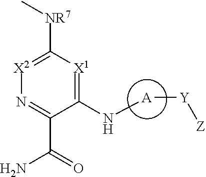

are attached to the same carbon atom or attached to carbon atoms that are adjacent to each other, and X¹ is N; then X² is other than CH.

In some embodiments, the compound is a compound of Formula (B-I) or (B-II), wherein the group

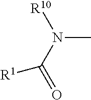

and the group

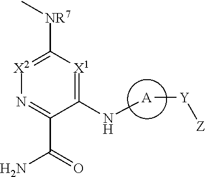

are not attached to the same carbon atom or attached to carbon atoms that are adjacent to each other.

In one aspect, provided herein is a compound of Formula (B-IA) having the structure:

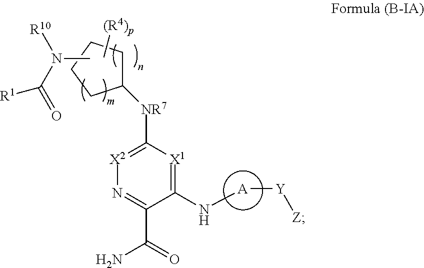

or a pharmaceutically acceptable solvate, pharmaceutically acceptable salt, or pharmaceutically acceptable prodrug thereof, wherein A, X¹, X², Y, Z, R¹, R⁴, R⁷, R¹⁰, n and p are as defined herein, and m is 1, 2, or 3.

In one aspect, provided herein is a compound of Formula (B-TB) having the structure:

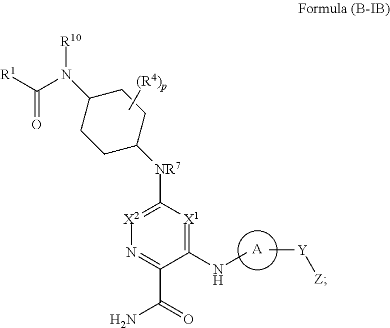

or a pharmaceutically acceptable solvate, pharmaceutically acceptable salt, or pharmaceutically acceptable prodrug thereof, wherein A, X¹, X², Y, Z, R¹, R⁴, R⁷, R¹⁰ and p are as defined herein.

In some embodiments, R² is hydrogen. In some embodiments, R² is C₃-C₄alkyl. In some embodiments, R² is other than ethyl.

In some embodiments, the compound is a compound of Formula (B-I), (B-II), (B-IA) or (B-IB) wherein R¹ is substituted or unsubstituted C₆-C₁₂aryl, or substituted or unsubstituted C₁-C₁₂heteroaryl. In some embodiments, the compound is a compound of Formula (B-I), (B-II), (B-IA) or (B-IB) wherein R¹ is substituted or unsubstituted phenyl, pyridyl, pyrimidinyl, indolyl, benzimidazolyl, or benzofuranyl. In some embodiments, the compound is a compound of Formula (B-I), (B-II), (B-IA) or (B-IB) wherein R¹ is substituted or unsubstituted isoindolinyl. In some embodiments, the compound is a compound of Formula (B-I), (B-II), (B-IA) or (B-IB) wherein R¹ is NR⁵R¹¹.

In some embodiments, the compound is a compound of Formula (B-I), (B-II), (B-IA) or (B-IB) wherein R¹ is substituted or unsubstituted C₂-C₄alkenyl or, substituted or unsubstituted C₂-C₄alkynyl. In some embodiments, the compound is a compound of Formula (B-I), (B-II), (B-IA) or (B-IB) wherein R¹ is unsubstituted C₂-C₄alkenyl or unsubstituted C₂-C₄alkynyl. In some embodiments, the compound is a compound of Formula (B-I), (B-II), (B-IA) or (B-IB) wherein R¹ is C₂-C₄alkenyl or C₂-C₄alkynyl substituted with OR¹⁷ or NR¹⁷R¹⁸, wherein R¹⁷ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl.

In one aspect, provided herein is a compound of Formula (C-I) having the structure:

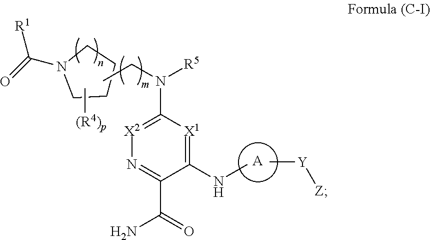

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, substituted or unsubstituted C₁-C₄alkyl or C(O)—(C₂-C₄alkenyl);
R⁷ is independently substituted or unsubstituted C₁-C₄alkyl, substituted or unsubstituted C₂-C₄alkenyl, substituted or unsubstituted C₂-C₄alkynyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹⁰ is H, or substituted or unsubstituted C₁-C₄alkyl;
m is 0 or 1;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when m is 0, then -A-Y—Z is other than

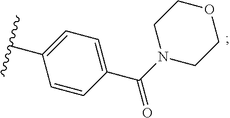 [SMILES: *c1ccc(C(=O)N2CCOCC2)cc1]

(2) when m is 0, A is quinolinyl, X¹ is N and X² is CH, then R¹ is other than Me;
(3) when R¹ is substituted or unsubstituted C₁-C₄alkyl or substituted or unsubstituted C₂-C₄alkenyl, m is 0, and X¹ is N, then X² is CH or N;
(4) the compound is other than
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide;
or
(R)-3-(5-carbamoyl-6-(3-methylisothiazol-5-ylamino)pyrazin-2-ylamino)-N,N-dimethylazepane-1-carboxamide;
or a pharmaceutically acceptable solvate, pharmaceutically acceptable salt, or pharmaceutically acceptable prodrug thereof.

In some embodiments, R⁵ is H or substituted or unsubstituted C₁-C₄alkyl.

In some embodiments, the compound is a compound of Formula (C-I), wherein A, Y, Z, R², R⁴, R⁵, R⁷, R¹⁰, m, n and p are as defined herein; X¹ is N and X² is C(R²); and R¹ is substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN.

In some embodiments, the compound is a compound of Formula (C-I), wherein A, Y, Z, R⁴, R⁵, R⁷, R¹⁰, m, n and p are as defined herein; each of X¹ and X² is N; and R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN.

In some embodiments, the compound is a compound of Formula (C-I), wherein X¹ and X² are both N or are both CH; or X¹ is —N— and X² is CH.

In one aspect, provided herein is a compound of Formula (C-I), (C-IA) or (C-IB) having the structure:

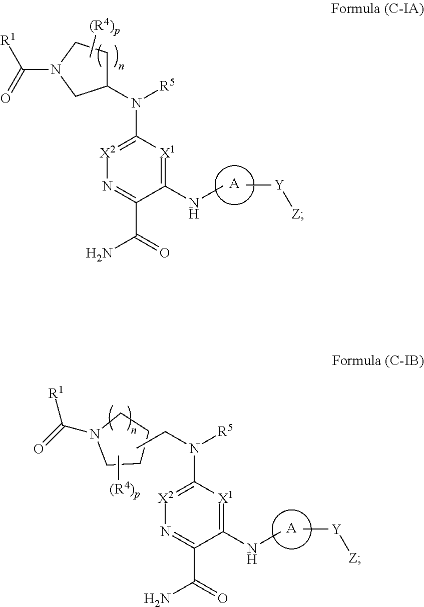

or a pharmaceutically acceptable solvate, pharmaceutically acceptable salt, or pharmaceutically acceptable prodrug thereof, wherein A, X¹, X², Y, Z, R¹, R⁴, R⁵, n and p are as defined herein.

In some embodiments, the compound is a compound of Formula (C-I), (C-IA) or (C-IB) wherein R¹ is substituted or unsubstituted C₆-C₁₂aryl, or substituted or unsubstituted C₁-C₁₂heteroaryl. In some embodiments, the compound is a compound of Formula (C-I), (C-IA) or (C-IB) wherein R¹ is substituted or unsubstituted phenyl, pyridyl, pyrimidinyl, indolyl, benzimidazolyl, or benzofuranyl. In some embodiments, the compound is a compound of Formula (C-I), (C-IA) or (C-IB) wherein R¹ is substituted or unsubstituted isoindolinyl. In some embodiments, the compound is a compound of Formula (C-I), (C-IA) or (C-IB) wherein R¹ is —NR⁷R¹⁰. In some embodiments, the compound is a compound of Formula (C-I), (C-IA) or (C-IB) wherein R¹ is —N(CH₃)₂.

In some embodiments, the compound is a compound of Formula (C-I), (C-IA) or (C-IB) wherein R¹ is substituted or unsubstituted C₂-C₄alkenyl or, substituted or unsubstituted C₂-C₄alkynyl. In some embodiments, the compound is a compound of Formula (C-I), (C-IA) or (C-IB) wherein R¹ is unsubstituted C₂-C₄alkenyl or unsubstituted C₂-C₄alkynyl. In some embodiments, the compound is a compound of Formula (C-I), (C-IA) or (C-IB) wherein R¹ is C₂-C₄alkenyl or C₂-C₄alkynyl substituted with OR¹⁷ or NR¹⁷R¹⁸, wherein R¹⁷ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl.

In some embodiments, R¹ is selected from CN,

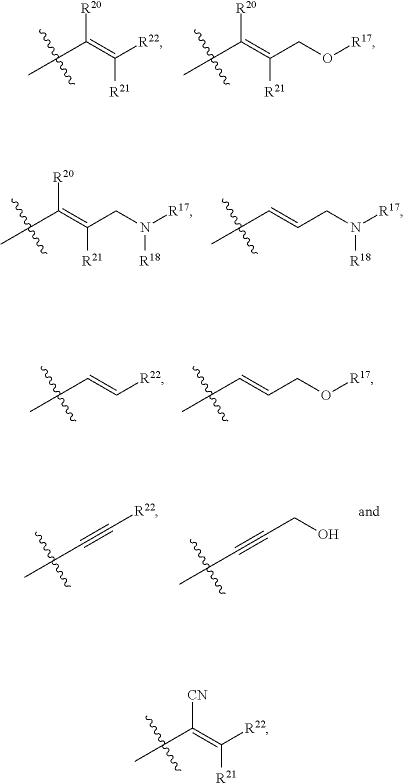

wherein R⁷ and R¹⁸ are as defined herein, R²⁰, R²¹ and R²² are each independently H, CN, halo, substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond.

In some embodiments, R¹ is selected from CN,

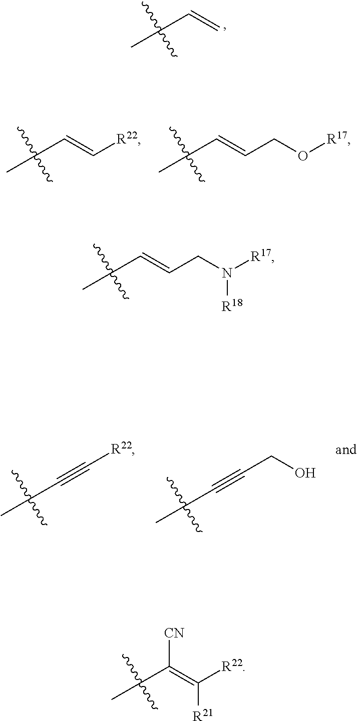

In some embodiments, R²⁰, R²¹ and R²² are independently H, F, Cl, C₁-C₄ alkyl or C₃-C₆ cycloalkyl, CF₃, or CN. In some embodiments, one of R²⁰ and R²¹ is H, the other one of R²⁰ and R²¹ is F, Cl, C₁-C₄ alkyl, C₃-C₈ cycloalkyl, CF₃, or CN, and R²² is H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl.

In some embodiments, R⁷ is H or substituted or unsubstituted C₁-C₄alkyl.

In another aspect, provided herein is a compound of Formula (A-IIa) having the structure:

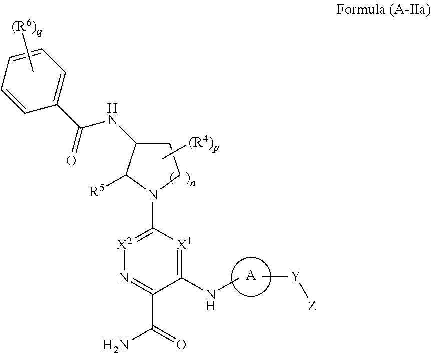

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂. R³ is as defined herein; and
q is 0, 1, 2 or 3;
or a pharmaceutically acceptable solvate, pharmaceutically acceptable salt, and/or pharmaceutically acceptable prodrug thereof.

In another aspect, provided herein is a compound of Formula (A-IIb) having the structure:

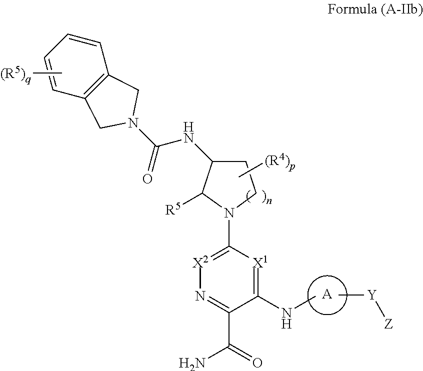

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₇heterocycloalkyl, or —N(R³)₂; R³ is as defined herein; and
q is 0, 1, 2 or 3;
or a pharmaceutically acceptable solvate, pharmaceutically acceptable salt, and/or pharmaceutically acceptable prodrug thereof.

In another aspect, provided herein is a compound of Formula (A-IIIa) having the structure:

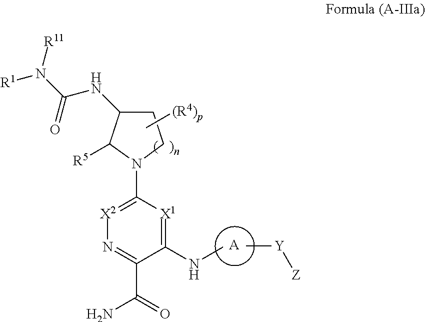

wherein:
A, X¹, X², Y, Z, R¹, R⁴, R⁵, R¹¹, n and p are as defined herein;
or a pharmaceutically acceptable solvate, pharmaceutically acceptable salt, and/or pharmaceutically acceptable prodrug thereof.

In another aspect, provided herein is a compound of Formula (A-IIIb) having the structure:

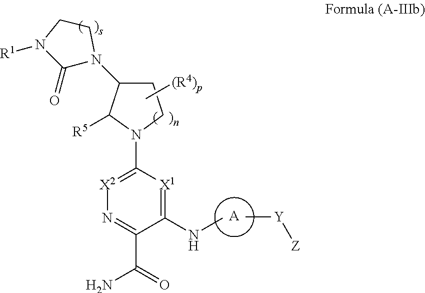

wherein:
A, X¹, X², Y, Z, R¹, R⁴, R⁵, n and p are as defined herein, and s is 1, 2 or 3;
or a pharmaceutically acceptable solvate, pharmaceutically acceptable salt, and/or pharmaceutically acceptable prodrug thereof.

In some embodiments, the present invention provides a compound of Formula (A-I), (A-I), (A-VI), (A-IA)-(A-IE), (A-IIa), (A-IIb), (A-IIIa) or (A-IIIb) wherein ring A is substituted or unsubstituted C₆-C₁₂aryl. In some embodiments, the compound is a compound of Formula (A-I), (A-II), (A-VI), (A-IA)-(A-IE), (A-IIa), (A-IIb), (A-IIIa) or (A-IIIb) wherein ring A is phenyl. In some embodiments, the compound is a compound of Formula (A-I), (A-II), (A-VI), (A-IA)-(A-IE), (A-IIa), (A-IIb), (A-IIIa) or (A-IIIb) wherein Y is a single bond, —CH₂O—, —OCH₂—, —O—, —N(R³)—, —C(O)—, —N(R₃)C(O)—, —C(O)N(R³)—, or substituted or unsubstituted C₁-C₄alkylene. In some embodiments, the compound is a compound of Formula (A-I), (A-II), (A-VI), (A-IA)-(A-IE), (A-IIa), (A-IIb), (A-IIIa) or (A-IIIb) wherein Y is a single bond, —CH₂O—, —OCH₂—, —O—, —N(R³)—, —N(R₃)C(O)—, —C(O)N(R³)—, or substituted or unsubstituted C₁-C₄alkylene. In some embodiments, the compound is a compound of Formula (A-I), (A-II), (A-VI), (A-IA)-(A-IE), (A-IIa), (A-IIb), (A-IIIa) or (A-IIIb) wherein Y is a single bond, —C(O)—, or —C(O)N(R³)—. In some embodiments, the compound is a compound of Formula (A-I), (A-II), (A-VI), (A-IA)-(A-IE), (A-IIa), (A-IIb), (A-IIIa) or (A-IIIb) wherein Z is substituted or unsubstituted C₁-C₃alkyl. In some embodiments, the compound is a compound of Formula (A-I), (A-II), (A-VI), (A-IA)-(A-IE), (A-IIa), (A-IIb), (A-IIIa) or (A-IIIb) wherein Z is Me, Et, or i-Pr. In some embodiments, the compound is a compound of Formula (A-I), (A-II), (A-IA)-(A-IE), (A-IIa), (A-IIb), (A-IIIa) or (A-IIIb) wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments, the present invention provides a compound of Formula (A-I), (A-II), (A-VI), (A-IA)-(A-IE), (A-IIa), (A-IIb), (A-IIIa) or (A-IIIb) wherein ring A is substituted or unsubstituted C₁-C₁₂heteroaryl. In some embodiments, the compound is a compound of Formula (A-I), (A-II), (A-VI), (A-IA)-(A-IE), (A-IIa), (A-IIb), (A-IIIa) or (A-IIIb) wherein ring A is pyridyl. In some embodiments, the compound is a compound of Formula (A-I), (A-II), (A-VI), (A-IA)-(A-IE), (A-IIa), (A-IIb), (A-IIIa) or (A-IIIb) wherein A is isothiazolyl. In some embodiments, the compound is a compound of Formula (A-I), (A-II), (A-VI), (A-IA), (A-IB), (A-IIa), (A-IIb), (A-IIIa) or (A-IIIb) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (A-I), (A-II), (A-VI), (A-IA)-(A-IE), (A-IIa), (A-IIb), (A-IIIa) or (A-IIIb) wherein Y is a single bond, —CH₂O—, —OCH₂—, —O—, —N(R³)—, —N(R₃)C(O)—, —C(O)N(R³)—, or substituted or unsubstituted C₁-C₄alkylene. In some embodiments, the compound is a compound of Formula (A-I), (A-II), (A-VI), (A-IA)-(A-IE), (A-IIa), (A-IIb), (A-IIIa) or (A-IIIb) wherein Y is a single bond, —C(O)—, or —C(O)N(R³)—. In some embodiments, the compound is a compound of Formula (A-I), (A-II), (A-VI), (A-IA)-(A-IE), (A-IIIa), (A-IIb), (A-IIIa) or (A-IIIb) wherein Z is substituted or unsubstituted C₁-C₃alkyl. In some embodiments, the compound is a compound of Formula (A-I), (A-II), (A-VI), (A-IA)-(A-IE), (A-IIIa), (A-IIb), (A-IIIa) or (A-IIIb) wherein Z is Me, Et, or i-Pr. In some embodiments, the compound is a compound of Formula (A-I), (A-II), (A-VI), (A-IA)-(A-IE), (A-IIa), (A-IIb), (A-IIIa) or (A-IIIb) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments, the compound is a compound of Formula (A-I), (A-II), (A-VI), (A-IA)-(A-IE), (A-IIa), (A-IIb), (A-IIIa) or (A-IIIb) wherein A is isothiazolyl; Y is a single bond; and Z is Me.

In further embodiments of the aforementioned embodiments the present invention provides a compound of Formula (A-I), (A-I), (A-VI), (A-IA)-(A-IE), (A-IIa), (A-IIb), (A-IIIa) or (A-IIIb) wherein X¹ and X² are both N. In further embodiments of the aforementioned embodiments the present invention provides a compound of Formula (A-I), (A-II), (A-VI), (A-IA)-(A-IE), (A-IIa), (A-IIb), (A-IIIa) or (A-IIIb) wherein X¹ and X² are both CH. In further embodiments of the aforementioned embodiments the present invention provides a compound of Formula (A-I), (A-II), (A-VI), (A-IA)-(A-IE), (A-IIa), (A-IIb), (A-IIIa) or (A-IIIb) wherein X¹ is N and X² is CH.

In some embodiments, the present invention provides a compound of Formula (A-I), (A-II), (A-VI), (A-IA)-(A-IE), (A-IIa), (A-IIb), (A-IIIa) or (A-IIIb) wherein X¹ and X² are both CH, and A is substituted or unsubstituted heteroaryl. In some embodiments, the present invention provides a compound of Formula (A-I), (A-II), (A-VI), (A-IA)-(A-IE), (A-IIa), (A-IIb), (A-IIIa) or (A-IIIb) wherein X¹ and X² are both CH, then A is substituted or unsubstituted heteroaryl.

In another aspect the present invention provides a pharmaceutical composition comprising a therapeutically effective amount of a compound of any one of formulas described herein or a pharmaceutically acceptable salt, pharmaceutically acceptable solvate, and/or pharmaceutically acceptable prodrug thereof, and a pharmaceutically acceptable excipient. In some embodiments, the pharmaceutical composition comprising the compound of any one of formulas described herein or a pharmaceutically acceptable salt, pharmaceutically acceptable solvate, and/or pharmaceutically acceptable prodrug thereof, is formulated for a route of administration selected from oral administration, parenteral administration, buccal administration, nasal administration, topical administration, or rectal administration.

In another aspect the present invention provides a method for treating an autoimmune disease or condition comprising administering to a patient in need a therapeutically effective amount of a compound of any one of formulas described herein or a pharmaceutically acceptable salt, pharmaceutically acceptable solvate, or and/pharmaceutically acceptable prodrug thereof. In some embodiments the autoimmune disease is selected from rheumatoid arthritis or lupus.

In another aspect the present invention provides a method for treating a heteroimmune disease or condition comprising administering to a patient in need a therapeutically effective amount of a compound of any one of formulas described herein or a pharmaceutically acceptable salt, pharmaceutically acceptable solvate, and/or pharmaceutically acceptable prodrug thereof.

In another aspect the present invention provides a method for treating a cancer comprising administering to a patient in need a therapeutically effective amount of a compound of any one of formulas described herein or a pharmaceutically acceptable salt, pharmaceutically acceptable solvate, and/or pharmaceutically acceptable prodrug thereof. In some embodiments the cancer is a B-cell proliferative disorder. In some embodiments the B-cell proliferative disorder is diffuse large B cell lymphoma, follicular lymphoma, mantle cell lymphoma, or chronic lymphocytic leukemia.

In another aspect the present invention provides a method for treating mastocytosis comprising administering to a patient in need a therapeutically effective amount of a compound of any one of formulas described herein or a pharmaceutically acceptable salt, pharmaceutically acceptable solvate, and/or pharmaceutically acceptable prodrug thereof.

In another aspect the present invention provides a method for treating osteoporosis or bone resorption disorders comprising administering to a patient in need a therapeutically effective amount of a compound of any one of formulas described herein or a pharmaceutically acceptable salt, pharmaceutically acceptable solvate, and/or pharmaceutically acceptable prodrug thereof.

In another aspect the present invention provides a method for treating an inflammatory disease or condition comprising administering to a patient in need a therapeutically effective amount of a compound of any one of formulas described herein or a pharmaceutically acceptable salt, pharmaceutically acceptable solvate, and/or pharmaceutically acceptable prodrug thereof.

In another aspect, provided herein is a compound of Formula (B-IIa) having the structure:

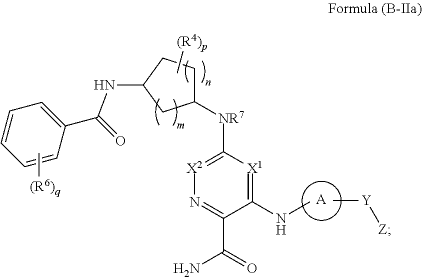

wherein:
A, X¹, X² Y, Z, R⁴, R⁷, m, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In another aspect, provided herein is a compound of Formula (B-IIb) having the structure:

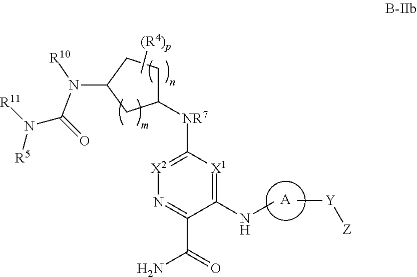

wherein:
A, X¹, X², Y, Z, R⁴, R⁵, R⁷, R¹⁰, R¹¹, m, n and p are as defined herein;
or a pharmaceutically acceptable solvate, pharmaceutically acceptable salt, or pharmaceutically acceptable prodrug thereof.

In another aspect, provided herein is a compound of Formula (B-IIc) having the structure:

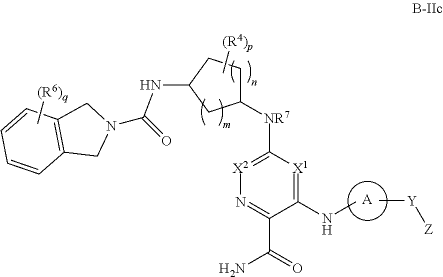

wherein:
A, X¹, X², Y, Z, R⁴, R⁷, m, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In another aspect, provided herein is a compound of Formula (B-IId) having the structure:

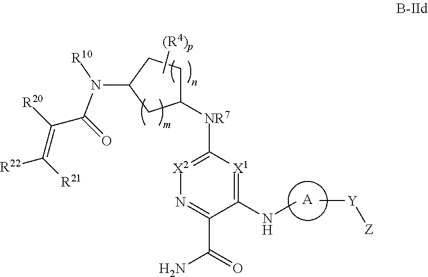

wherein:
A, X¹, X², Y, Z, R⁴, R⁷, R¹⁰, m, n and p are as defined herein:
R²⁰, R²¹ and R²² are each independently H, CN, halo, substituted or unsubstituted C₁-C₄alkyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, or pharmaceutically acceptable prodrug thereof.

In some embodiments, the present invention provides a compound of Formula (B-IId), wherein R²⁰ and R²¹ are H, R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, all of R²⁰, R²¹ and R²² are H. In some embodiments, R²⁰ and R²¹ together form a bond and R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R²⁰ is CN. In some embodiments, R²⁰ is halo, such as F or Cl.

In one aspect, provided herein is a compound of Formula (B-VIII) having the structure:

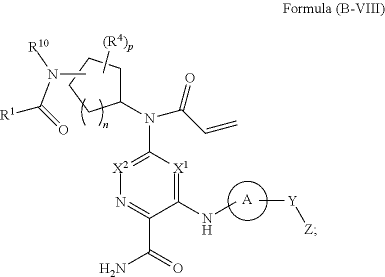

or a pharmaceutically acceptable solvate, pharmaceutically acceptable salt, or pharmaceutically acceptable prodrug thereof, the variables are as defined herein.

In some embodiments, the present invention provides a compound of Formula (B-I), (B-II), (B-IA), (B-IB), (B-IIa)-(B-IId) or (B-VIII) wherein ring A is substituted or unsubstituted C₆-C₁₂aryl. In some embodiments, the compound is a compound of Formula (B-I), (B-II), (B-IA), (B-IB), (B-IIa)-(B-IId) or (B-VIII) wherein ring A is phenyl. In some embodiments, the compound is a compound of Formula (B-I), (B-II), (B-IA), (B-IB), (B-IIa)-(B-IId) or (B-VIII) wherein Y is a single bond, —CH₂O—, —OCH₂—, —O—, —N(R³)—, —C(O)—, —N(R₃)C(O)—, —C(O)N(R³)—, or substituted or unsubstituted C₁-C₄alkylene. In some embodiments, the compound is a compound of Formula (B-I), (B-II), (B-IA), (B-IB), (B-IIa)-(B-IId) or (B-VIII) wherein Y is a single bond, —C(O)—, or —C(O)N(R³)—. In some embodiments, the compound is a compound of Formula (B-I), (B-II), (B-IA), (B-IB), (B-IIa)-(B-IId) or (B-VIII) wherein Z is substituted or unsubstituted C₁-C₃alkyl. In some embodiments the compound is a compound of Formula (B-I), (B-II), (B-IA), (B-IB), (B-Ia)-(B-IId) or (B-VIII) wherein Z is Me, Et, or i-Pr. In some embodiments, the compound is a compound of Formula (B-I), (B-II), (B-IA), (B-IB), (B-IIa)-(B-IId) or (B-VIII) wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments, the present invention provides a compound of Formula (B-I), (B-II), (B-IA), (B-IB), (B-IIa)-(B-IId) or (B-VIII) wherein ring A is substituted or unsubstituted C₁-C₁₂heteroaryl. In some embodiments, the compound is a compound of Formula (B-I), (B-II), (B-IA), (B-IB), (B-IIa)-(B-IId) or (B-VIII) wherein ring A is pyridyl. In some embodiments, the compound is a compound of Formula (B-I), (B-II), (B-IA), (B-IB), (B-IIa)-(B-IId) or (B-VIII) wherein A is isothiazolyl. In some embodiments, the compound is a compound of Formula (B-I), (B-II), (B-IA), (B-IB), (B-IIa)-(B-IId) or (B-VIII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (B-I), (B-II), (B-IA), (B-IB), (B-IIa)-(B-IId) or (B-VIII) wherein Y is a single bond, —C(O)—, or —C(O)N(R³)—. In some embodiments, the compound is a compound of Formula (B-I), (B-II), (B-IA), (B-IB), (B-IIa)-(B-IId) or (B-VIII) wherein Z is substituted or unsubstituted C₁-C₃alkyl. In some embodiments, the compound is a compound of Formula (B-I), (B-II), (B-IA), (B-IB), (B-IIa)-(B-IId) or (B-VIII) wherein Z is Me, Et, or i-Pr. In some embodiments, the compound is a compound of Formula (B-I), (B-II), (B-IA), (B-IB), (B-IIa)-(B-IId) or (B-VIII) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments, the compound is a compound of Formula (B-I), (B-II), (B-IA), (B-IB), (B-IIa)-(B-IId) or (B-VIII) wherein A is isothiazolyl; Y is a single bond; and Z is Me.

In some embodiments, the present invention provides a compound of Formula (B-I), (B-II), (B-IA), (B-IB), (B-IIa)-(B-IId) or (B-VIII) wherein X¹ and X² are both N. In some embodiments, the compound is a compound of Formula (B-I), (B-II), (B-IA), (B-IB), (B-IIa)-(B-IId) or (B-VIII) wherein X¹ and X² are independently C(R²). In some embodiments, the compound is a compound of Formula (B-I), (B-II), (B-IA), (B-IB), (B-IIa)-(B-IId) or (B-VIII) wherein X¹ is N and X² is C(R²). In some embodiments, each R² is independently H, substituted or unsubstituted C₁-C₄alkyl, —CN, or halogen. In some embodiments, R² is H.

In another aspect the present invention provides a pharmaceutical composition comprising a therapeutically effective amount of a compound of any one of formulas described herein or a pharmaceutically acceptable salt, pharmaceutically acceptable solvate, or pharmaceutically acceptable prodrug thereof, and a pharmaceutically acceptable excipient. In some embodiments, the pharmaceutical composition comprising the compound of any one of formulas described herein or a pharmaceutically acceptable salt, pharmaceutically acceptable solvate, or pharmaceutically acceptable prodrug thereof, is formulated for a route of administration selected from oral administration, parenteral administration, buccal administration, nasal administration, topical administration, or rectal administration.

In another aspect the present invention provides a method for treating an autoimmune disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof. In some embodiments the autoimmune disease is selected from rheumatoid arthritis or lupus.

In another aspect the present invention provides a method for treating a heteroimmune disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof.

In another aspect the present invention provides a method for treating a cancer comprising administering to a patient in need a therapeutically effective amount of a compound of any one of formulas described herein or a pharmaceutically acceptable salt, pharmaceutically acceptable solvate, or pharmaceutically acceptable prodrug thereof. In some embodiments the cancer is a B-cell proliferative disorder. In some embodiments the B-cell proliferative disorder is diffuse large B cell lymphoma, follicular lymphoma, mantle cell lymphoma, or chronic lymphocytic leukemia.

In another aspect the present invention provides a method for treating mastocytosis comprising administering to a patient in need a therapeutically effective amount of a compound of any one of formulas described herein or a pharmaceutically acceptable salt, pharmaceutically acceptable solvate, or pharmaceutically acceptable prodrug thereof.

In another aspect another aspect the present invention provides a method for treating osteoporosis or bone resorption disorders comprising administering to a patient in need a therapeutically effective amount of a compound of any one of formulas described herein or a pharmaceutically acceptable salt, pharmaceutically acceptable solvate, or pharmaceutically acceptable prodrug thereof.

In a another aspect the present invention provides a method for treating an inflammatory disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof.

In one aspect, provided herein is a compound of Formula (C-IC) having the structure:

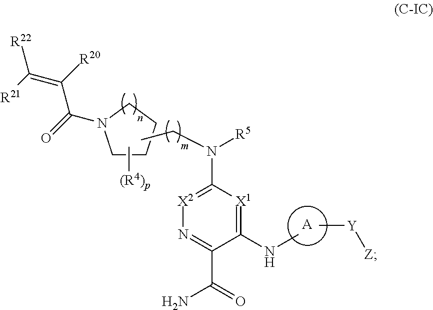

wherein:
A, X¹, X², Y, Z, R⁴, R⁵, m, n and p are as defined herein;
R²⁰, R²¹ and R²² are each independently H, CN, halo, substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, or pharmaceutically acceptable prodrug thereof.

In some embodiments, R²⁰ and R²¹ are H, R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, all of R²⁰, R²¹ and R²² are H. In some embodiments, R²⁰ and R²¹ together form a bond and R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R²⁰ is methyl or CN. In some embodiments, R²⁰ is halo, such as F or Cl.

In another aspect, provided herein is a compound of Formula (C-IIa) having the structure:

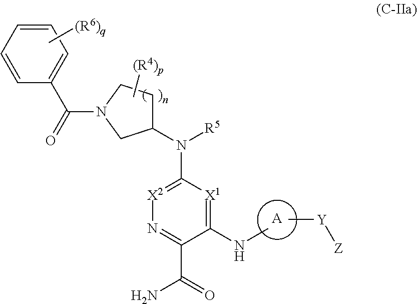

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3
or a pharmaceutically acceptable solvate, pharmaceutically acceptable salt, or pharmaceutically acceptable prodrug thereof.

In another aspect, provided herein is a compound of Formula (C-IIb) having the structure:

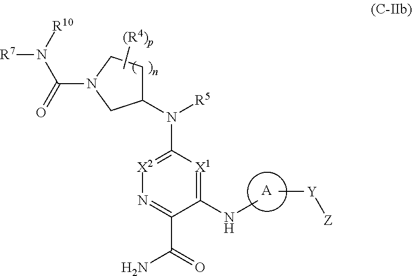

wherein:
A, X¹, X², Y, Z, R⁴, R⁵, R⁷, R¹⁰, n and p are as defined herein;
or a pharmaceutically acceptable solvate, pharmaceutically acceptable salt, or pharmaceutically acceptable prodrug thereof.

In another aspect, provided herein is a compound of Formula (C-IIc) having the structure:

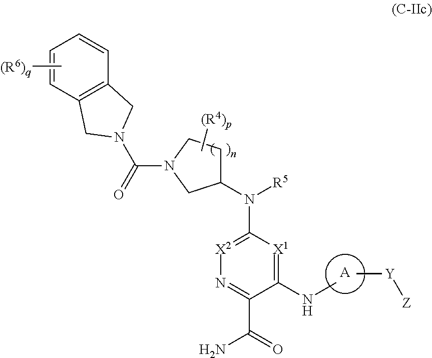

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In another aspect, provided herein is a compound of Formula (C-IIIa) having the structure:

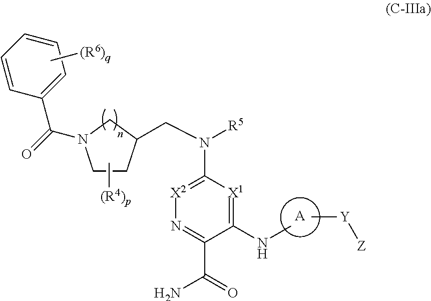

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₇heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In another aspect, provided herein is a compound of Formula (C-IIIb) having the structure:

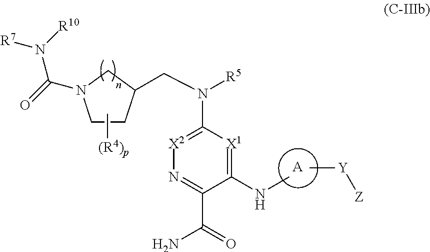

wherein:
A, X¹, X², Y, Z, R⁵, R⁷, R¹⁰, n and p are as defined herein;
or a pharmaceutically acceptable solvate, pharmaceutically acceptable salt, or pharmaceutically acceptable prodrug thereof.

In another aspect, provided herein is a compound of Formula (C-IIIc) having the structure:

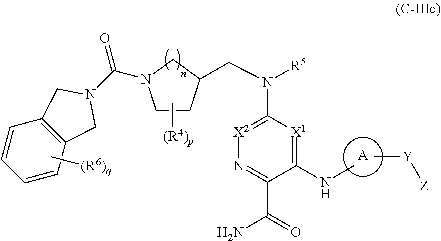

wherein:
A, X¹, X², Y, Z, R⁴, R⁵, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In one aspect, provided herein is a compound of Formula (C-VIII) having the structure:

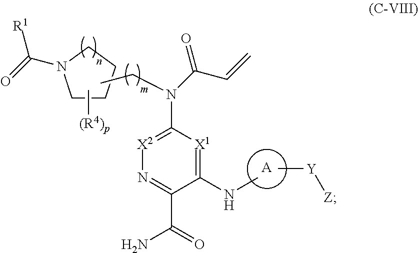

or a pharmaceutically acceptable solvate, pharmaceutically acceptable salt, or pharmaceutically acceptable prodrug thereof, wherein the variables are as defined herein.

In some embodiments, the present invention provides a compound of Formula (C-I), (C-IA)-(C-IC), (C-IIa)-(C-IIc), (C-IIIa)-(C-IIIc), or (C-VIII), wherein ring A is substituted or unsubstituted C₆-C₁₂aryl. In some embodiments, the compound is a compound of Formula (C-I), (C-IA)-(C-IC), (C-IIa)-(C-IIc), (C-IIIa)-(C-IIIc), or (C-VIII), wherein ring A is phenyl. In some embodiments, the compound is a compound of Formula (C-I), (C-IA)-(C-IC), (C-IIa)-(C-IIc), (C-IIIa)-(C-IIIc), or (C-VIII), wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (C-I), (C-IA)-(C-IC), (C-IIa)-(C-IIc), (C-IIIa)-(C-IIc), or (C-VIII), wherein Y is a single bond, —C(O)—, or —C(O)N(R³)—. In some embodiments, the compound is a compound of Formula (C-I), (C-IA)-(C-IC), (C-IIa)-(C-IIc), (C-IIIa)-(C-IIIc), or (C-VIII), wherein Z is substituted or unsubstituted C₁-C₃alkyl. In some embodiments, the compound is a compound of Formula (C-I), (C-IA)-(C-IC), (C-IIa)-(C-IIc), (C-IIIa)-(C-IIIc), or (C-VIII), wherein Z is Me, Et, or i-Pr. In some embodiments, the compound is a compound of Formula (C-I), (C-IA)-(C-IC), (C-IIa)-(C-IIc), (C-IIIa)-(C-IIIc), or (C-VIII), wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments, the present invention provides a compound of Formula (C-I), (C-IA)-(C-IC), (C-IIa)-(C-IIc), (C-IIIa)-(C-IIIc), or (C-VIII), wherein ring A is substituted or unsubstituted C₁-C₁₂heteroaryl. In some embodiments, the compound is a compound of Formula (C-I), (C-IA)-(C-IC), (C-IIa)-(C-IIc), (C-IIIa)-(C-IIIc), or (C-VIII), wherein ring A is pyridyl. In some embodiments, the compound is a compound of Formula (C-I), (C-IA)-(C-IC), (C-IIa)-(C-IIc), (C-IIIa)-(C-IIIc), or (C-VIII), wherein A is isothiazolyl. In some embodiments, the compound is a compound of Formula (C-I), (C-IA)-(C-IC), (C-IIa)-(C-IIc), (C-IIIa)-(C-IIIc), or (C-VIII), wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (C-I), (C-IA)-(C-IC), (C-IIIa)-(C-IIIc), (C-IIIa)-(C-IIIc), or (C-VIII) wherein Y is a single bond, —C(O)—, or —C(O)N(R³)—. In some embodiments, the compound is a compound of Formula (C-I), (C-IA)-(C-IC), (C-IIIa)-(C-IIIc), (C-IIIa)-(C-IIIc), or (C-VIII) wherein Z is substituted or unsubstituted C₁-C₃alkyl. In some embodiments, the compound is a compound of Formula (C-I), (C-IA)-(C-IC), (C-IIIa)-(C-IIIc), (C-IIIa)-(C-IIIc), or (C-VIII) wherein Z is Me, Et, or i-Pr. In some embodiments, the compound is a compound of Formula (C-I), (C-IA)-(C-IC), (C-IIa)-(C-IIc), (C-IIIa)-(C-IIIc), or (C-VIII) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments, the compound is a compound of Formula (C-I), (C-IA)-(C-IC), (C-IIa)-(C-IIc), (C-IIIa)-(C-IIIc), or (C-VIII) wherein A is isothiazolyl; Y is a single bond; and Z is Me.

In further embodiments of the aforementioned embodiments, the compound is a compound of Formula (C-I), (C-IA)-(C-IC), (C-IIa)-(C-IIc), (C-IIIa)-(C-IIIc), or (C-VIII) wherein X¹ and X² are both N. In further embodiments of the aforementioned embodiments, the compound is a compound of Formula (C-I), (C-IA)-(C-IC), (C-IIa)-(C-IIc), (C-IIIa)-(C-IIIc), or (C-VIII) wherein X¹ and X² are independently C(R²). In further embodiments of the aforementioned embodiments, the compound is a compound of Formula (C-I), (C-IA)-(C-IC), (C-IIa)-(C-IIc), (C-IIIa)-(C-IIIc), or (C-VIII) wherein X¹ is N and X² is C(R²). In some embodiments, R² is H.

In another aspect the present invention provides a pharmaceutical composition comprising a therapeutically effective amount of a compound of any one of formulas described herein or a pharmaceutically acceptable salt, pharmaceutically acceptable solvate, or pharmaceutically acceptable prodrug thereof, and a pharmaceutically acceptable excipient. some embodiments, the pharmaceutical composition comprising the compound of any one of formulas described herein or a pharmaceutically acceptable salt, pharmaceutically acceptable solvate, or pharmaceutically acceptable prodrug thereof, is formulated for a route of administration selected from oral administration, parenteral administration, buccal administration, nasal administration, topical administration, or rectal administration.

In another aspect the present invention provides a method for treating an autoimmune disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof. In some embodiments the autoimmune disease is selected from rheumatoid arthritis or lupus.

In another aspect the present invention provides a method for treating a heteroimmune disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof.

In another aspect the present invention provides a method for treating a cancer comprising administering to a patient in need a therapeutically effective amount of a compound of any one of formulas described herein or a pharmaceutically acceptable salt, pharmaceutically acceptable solvate, or pharmaceutically acceptable prodrug thereof. In some embodiments the cancer is a B-cell proliferative disorder. In some embodiments the B-cell proliferative disorder is diffuse large B cell lymphoma, follicular lymphoma, mantle cell lymphoma, or chronic lymphocytic leukemia.

In another aspect the present invention provides a method for treating mastocytosis comprising administering to a patient in need a therapeutically effective amount of a compound of any one of formulas described herein or a pharmaceutically acceptable salt, pharmaceutically acceptable solvate, or pharmaceutically acceptable prodrug thereof.

In another aspect the present invention provides a method for treating osteoporosis or bone resorption disorders comprising administering to a patient in need a therapeutically effective amount of a compound of any one of formulas described herein or a pharmaceutically acceptable salt, pharmaceutically acceptable solvate, or pharmaceutically acceptable prodrug thereof.

In another aspect the present invention provides a method for treating an inflammatory disease or condition comprising administering to a patient in need a therapeutically effective amount of a compound of any one of formulas described herein or a pharmaceutically acceptable salt, pharmaceutically acceptable solvate, or pharmaceutically acceptable prodrug thereof.

Any combination of the groups described above for the various variables is contemplated herein. It is understood that the compounds provided herein can be synthesized by techniques known in the art, as well as those set forth herein.

In a further aspect are provided pharmaceutical compositions, which include a therapeutically effective amount of at least one of any of the compounds herein, or a pharmaceutically acceptable salt, pharmaceutically acceptable prodrug, or pharmaceutically acceptable solvate. In certain embodiments, compositions provided herein further include a pharmaceutically acceptable diluent, excipient and/or binder.

Pharmaceutical compositions may be formulated for administration by an appropriate route and means containing effective concentrations of one or more of the compounds provided herein, or pharmaceutically effective derivatives thereof, that deliver amounts effective for the treatment, prevention, or amelioration of one or more symptoms of diseases, disorders or conditions that are modulated or otherwise affected by tyrosine kinase activity, or in which tyrosine kinase activity is implicated, are provided. The effective amounts and concentrations are effective for ameliorating any of the symptoms of any of the diseases, disorders or conditions disclosed herein.

In certain embodiments, provided herein is a pharmaceutical composition containing: i) a physiologically acceptable carrier, diluent, and/or excipient; and ii) one or more compounds provided herein.

In one aspect, provided herein are methods for treating a patient by administering a compound provided herein. In some embodiments, provided herein is a method of inhibiting the activity of tyrosine kinase(s), such as Btk, or of treating a disease, disorder, or condition, which would benefit from inhibition of tyrosine kinase(s), such as Btk, in a patient, which includes administering to the patient a therapeutically effective amount of at least one of any of the compounds herein, or pharmaceutically acceptable salt, pharmaceutically acceptable prodrug, or pharmaceutically acceptable solvate.

In another aspect, provided herein is the use of a compound disclosed herein for inhibiting Bruton's tyrosine kinase (Btk) activity or for the treatment of a disease, disorder, or condition, which would benefit from inhibition of Bruton's tyrosine kinase (Btk) activity.

In some embodiments, compounds provided herein are administered to a human.

In some embodiments, compounds provided herein are orally administered.

In some embodiments, compounds provided herein are used for the formulation of a medicament for the inhibition of tyrosine kinase activity. In some other embodiments, compounds provided herein are used for the formulation of a medicament for the inhibition of Bruton's tyrosine kinase (Btk) activity.

Articles of manufacture including packaging material, a compound or composition or pharmaceutically acceptable derivative thereof provided herein, which is effective for inhibiting the activity of tyrosine kinase(s), such as Btk, within the packaging material, and a label that indicates that the compound or composition, or pharmaceutically acceptable salt, pharmaceutically acceptable prodrug, or pharmaceutically acceptable solvate thereof, is used for inhibiting the activity of tyrosine kinase(s), such as Btk, are provided.

In a further aspect, provided herein is a method for inhibiting Bruton's tyrosine kinase in a subject in need thereof by administering to the subject a composition containing a therapeutically effective amount of a compound described herein. In some embodiments, the subject in need is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In some embodiments, the subject in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In some embodiments, the subject in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, the subject in need is suffering from a cancer. In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenström macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds. In some embodiments, the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002.

In some embodiments, the subject in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In another aspect, provided herein is a method for treating an autoimmune disease by administering to a subject in need thereof a composition containing a therapeutically effective amount of a compound described herein. In some embodiments, the autoimmune disease is arthritis. In some embodiments, the autoimmune disease is lupus. In some embodiments, the autoimmune disease is inflammatory bowel disease (including Crohn's disease and ulcerative colitis), rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behçet's disease, chronic fatigue, dysautonomia, endometriosis, interstitial cystitis, neuromyotonia, scleroderma, or vulvodynia.

In another aspect, provided herein is a method for treating a heteroimmune condition or disease by administering to a subject in need thereof a composition containing a therapeutically effective amount of a compound described herein. In some embodiments, the heteroimmune condition or disease is graft versus host disease, transplantation, transfusion, anaphylaxis, allergy, type I hypersensitivity, allergic conjunctivitis, allergic rhinitis, or atopic dermatitis.

In another aspect, provided herein is a method for treating an inflammatory disease by administering to a subject in need thereof a composition containing a therapeutically effective amount of a compound described herein. In some embodiments, the inflammatory disease is asthma, inflammatory bowel disease (including Crohn's disease and ulcerative colitis), appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In another aspect, provided herein is a method for treating a cancer by administering to a subject in need thereof a composition containing a therapeutically effective amount of a compound described herein. In some embodiments, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, where the subject is suffering from a cancer, an anti-cancer agent is administered to the subject in addition to one of the above-mentioned compounds. In some embodiments, the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002.

In another aspect, provided herein is a method for treating a thromboembolic disorder by administering to a subject in need thereof a composition containing a therapeutically effective amount of a compound described herein. In some embodiments, the thromboembolic disorder is myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In another aspect, provided herein is a method for treating an autoimmune disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In further or alternative embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a heteroimmune condition or disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In further or alternative embodiments, the compound forms a covalent bond with a cysteine residue on Bruton's tyrosine kinase.

In another aspect, provided herein is a method for treating an inflammatory disease by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a cancer by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein is a method for treating a thromboembolic disorder by administering to a subject in need thereof a composition containing a therapeutically effective amount of a compound that forms a covalent bond with Bruton's tyrosine kinase. In some embodiments, the compound forms a covalent bond with the activated form of Bruton's tyrosine kinase. In some embodiments, the compound irreversibly inhibits the Bruton's tyrosine kinase to which it is covalently bound. In some embodiments, the compound forms a covalent bond with a cysteine residue on Bruton's tyrosine kinase.

In another aspect, provided herein are methods for modulating, including irreversibly inhibiting, the activity of Btk or other tyrosine kinases, wherein the other tyrosine kinases share homology with Btk by having a cysteine residue (including a Cys 481 residue) that can form a covalent bond with at least one irreversible inhibitor described herein, in a mammal comprising administering to the mammal at least once an effective amount of a compound described herein. In another aspect, provided herein are methods for modulating, including reversibly or irreversibly inhibiting, the activity of Btk in a mammal comprising administering to the mammal at least once an effective amount of a compound described herein. In another aspect, provided herein are methods for treating Btk-dependent or Btk mediated conditions or diseases, comprising administering to the mammal at least once an effective amount of a compound described herein.

In another aspect, provided herein are methods for treating inflammation comprising administering to the mammal at least once an effective amount of a compound described herein.

In another aspect, provided herein are methods for the treatment of cancer comprising administering to the mammal at least once an effective amount of a compound described herein. The type of cancer may include, but is not limited to, pancreatic cancer and other solid or hematological tumors.

In another aspect, provided herein are methods for treating respiratory diseases comprising administering to the mammal at least once an effective amount of a compound described herein. In some embodiments, the respiratory disease is asthma. In some embodiments, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, and seasonal asthma.

In another aspect, provided herein are methods for preventing rheumatoid arthritis and osteoarthritis comprising administering to the mammal at least once an effective amount of a compound described herein.

In another aspect, provided herein are methods for treating inflammatory responses of the skin comprising administering to the mammal at least once an effective amount of a compound described herein. Such inflammatory responses of the skin include, by way of example, dermatitis, contact dermatitis, eczema, urticaria, rosacea, and scarring.

In another aspect, provided herein are methods for reducing psoriatic lesions in the skin, joints, or other tissues or organs, comprising administering to the mammal an effective amount of a first compound described herein.

In another aspect, provided herein is the use of a compound described herein, in the manufacture of a medicament for treating an inflammatory disease or condition in an animal in which the activity of Btk or other tyrosine kinases, wherein the other tyrosine kinases share homology with Btk by having a cysteine residue (including a Cys 481 residue) that can form a covalent bond with at least one irreversible inhibitor described herein, contributes to the pathology and/or symptoms of the disease or condition. In some embodiments, the tyrosine kinase protein is Btk. In other or further embodiments of this aspect, the inflammatory disease or conditions are respiratory, cardiovascular, or proliferative diseases.

In any of the aforementioned aspects are further embodiments in which administration is enteral, parenteral, or both, and embodiments wherein (a) the effective amount of the compound is systemically administered to the mammal; (b) the effective amount of the compound is administered orally to the mammal; (c) the effective amount of the compound is intravenously administered to the mammal; (d) the effective amount of the compound administered by inhalation; (e) the effective amount of the compound is administered by nasal administration; or (f) the effective amount of the compound is administered by injection to the mammal; (g) the effective amount of the compound is administered topically (dermal) to the mammal; (h) the effective amount of the compound is administered by ophthalmic administration; or (i) the effective amount of the compound is administered rectally to the mammal.

In any of the aforementioned aspects are further embodiments comprising single administrations of the effective amount of the compound, including further embodiments in which (i) the compound is administered to the mammal once; (ii) the compound is administered to the mammal multiple times over the span of one day; (iii) the compound is administered to the mammal continually; or (iv) the compound is administered to the mammal continuously.

In any of the aforementioned aspects are further embodiments comprising multiple administrations of the effective amount of the compound, including further embodiments in which (i) the time between multiple administrations is every 6 hours; (ii) the compound is administered to the mammal every 8 hours. In some embodiments, the method comprises a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In any of the aforementioned aspects involving the treatment of proliferative disorders, including cancer, are further embodiments comprising administering at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

In any of the aforementioned aspects involving the prevention or treatment of Btk-dependent or tyrosine kinase mediated diseases or conditions are further embodiments comprising identifying patients by screening for a tyrosine kinase gene haplotype. In further or alternative embodiments the tyrosine kinase gene haplotype is a tyrosine kinase pathway gene, while in still further or alternative embodiments, the tyrosine kinase gene haplotype is a Btk haplotype.

In a some embodiments, the compounds of the present invention are reversible inhibitors of Bruton's tyrosine kinase (Btk), while in some embodiments, such reversible inhibitors are selective for Btk. In some embodiments, such inhibitors have an IC₅₀ below 10 microM in enzyme assay. In some embodiments, a Btk reversible inhibitor has an IC₅₀ of less than 1 μM, and in some embodiments, less than 0.25 μM.

In some embodiments, the compounds of the present invention are selective reversible inhibitors for Btk over Itk. In some embodiments, the compounds of the present invention are selective reversible inhibitors for Btk over Lck. In some embodiments, the compounds of the present invention are selective reversible inhibitors for Btk over ABL. In some embodiments, the compounds of the present invention are selective reversible inhibitors for Btk over CMET. In some embodiments, the compounds of the present invention are selective reversible inhibitors for Btk over EGFR. In some embodiments, the compounds of the present invention are selective reversible inhibitors for Btk over Lyn.

In some embodiments, the reversible Btk inhibitors are also inhibitors of EGFR

In some embodiments, the compounds of the present invention are irreversible inhibitors of Bruton's tyrosine kinase (Btk), while in some embodiments, such irreversible inhibitors are selective for Btk. In some embodiments, such inhibitors have an IC₅₀ below 10 IM in enzyme assay. In some embodiments, such inhibitors have an IC₅₀ of less than 1 μM, and in some embodiments, less than 0.25 μM.

In some embodiments, the compounds of the present invention are selective irreversible inhibitors for Btk over Itk. In some embodiments, the compounds of the present invention are selective irreversible inhibitors for Btk over Lck. In some embodiments, the compounds of the present invention are selective irreversible inhibitors for Btk over ABL. In some embodiments, the compounds of the present invention are selective irreversible inhibitors for Btk over CMET.

In some embodiments, the compounds of the present invention are selective irreversible inhibitors for Btk over EGFR In some embodiments, the compounds of the present invention are selective irreversible inhibitors for Btk over Lyn.

In some embodiments, the irreversible Btk inhibitors are also inhibitors of EGFR.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the present disclosure will become apparent to those skilled in the art from this detailed description. The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise.

Furthermore, use of the term “including” as well as other forms, such as “include”, “includes,” and “included,” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl).

The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl or t-Bu), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, trimethylsilanyl, —OR, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms.

In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜOR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{c}-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is optionally saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR³ (where t is 1 or 2) and —R^{b}—S(O)₁N(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and Re is a straight or branched alkylene or alkenylene chain, and where each of the above substituents is unsubstituted unless otherwise indicated.

“Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine ring atoms. Cₓheterocycloalkyl refers to a heterocycloalkyl having x number of ring carbon atoms wherein the remaining ring atom(s) are heteroatom(s). Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

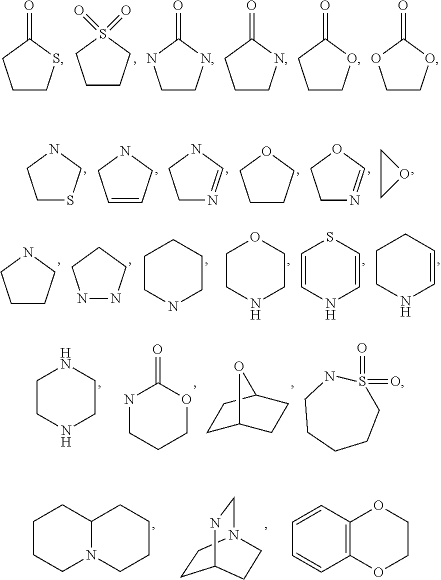

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises at least one heteroatom, in particular, one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system contains a heteroatom and is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. Cₓheteroaryl refers to a heteroaryl having x number of ring carbon atoms wherein the remaining ring atom(s) are heteroatom(s). The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—SR^{a}, —R^{b}—OC(O)—R^{a}, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to an (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl,” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule.

Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule.

Chemical moieties are often recognized chemical entities embedded in or appended to a molecule.

A “thioalkoxy” or “alkylthio” group refers to a —S-alkyl group.

An “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to an (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, supra.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, haloalkoxy, amino, including mono- and di-substituted amino groups, and the N-oxide and protected derivatives thereof; or “optionally substituted” or “substituted” may be -L^{s}R^{s}, wherein each L^{s} is independently selected from a single bond, —O—, —C(═O)—, —S—, —S(═O)—, —S(═O)₂—, —NH—, —N(CH₃)—, —NHC(O)—, —N(CH₃)C(O)—, —C(O)NH—, —C(O)N(CH₃)—, S(═O)₂NH—, —NHS(═O)₂—, —OC(O)NH—, —NHC(O)O—, -(substituted or unsubstituted C₁-C₆ alkyl)-, or -(substituted or unsubstituted C₂-C₆ alkenyl)-; and each R^{s} is independently selected from H, C₁-C₆alkyl, C₂-C₆ alkenyl, C₃-C₆cycloalkyl, C₂-C₇heterocycloalkyl, C₆-C₁₂aryl, C₁-C₁₂heteroaryl, or C₁-C₆heteroalkyl. The protecting groups that may form the protective derivatives of the above substituents are known to those of skill in the art and may be found in references such as Greene and Wuts, above.

The term “nucleophile” or “nucleophilic” refers to an electron rich compound, or moiety thereof. An example of a nucleophile includes, but in no way is limited to, a cysteine residue of a molecule, such as, for example Cys 481 of Btk.

The term “electrophile” or “electrophilic” refers to an electron poor or electron deficient molecule, or moiety thereof. Examples of electrophiles include, but in no way are limited to, Michael acceptor moieties.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, the term “agonist” refers to a compound, the presence of which results in a biological activity of a protein that is the same as the biological activity resulting from the presence of a naturally occurring ligand for the protein, such as, for example, Btk.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, Btk. In certain embodiments, an antagonist is an inhibitor.

As used herein, “amelioration” of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of the weight of compounds disclosed herein, such as, compounds of the present invention, dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which compounds disclosed herein, such as, compounds of any of Formula (A-I), (ii), (VI), (IA), (IB), (IIa), (IIb), (IIIa) or (IIIb) or Formula (B-I), (B-II), (B-IA), (B-IB), (B-IIa)-(B-IId) or (B-VIII), or Formula (C-I), (C-IA)-(C-IC), (C-IIa)-(C-IIc), (C-IIIa)-(C-IIIc), or (C-VIII), (C-IIIa)-(C-IIIc), or (C-VIII), or any other Formula described herein are absorbed into the general circulation when the pharmaceutical composition is taken orally as compared to intravenous injection.

“Blood plasma concentration” refers to the concentration of compounds disclosed herein, such as, compounds of the present invention, in the plasma component of blood of a subject. It is understood that the plasma concentration of compounds of the present invention, may vary significantly between subjects, due to variability with respect to metabolism and/or possible interactions with other therapeutic agents. In accordance with one embodiment disclosed herein, the blood plasma concentration of the compounds of the present invention, may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound of the present invention, may vary from subject to subject.

The term “Bruton's tyrosine kinase,” as used herein, refers to Bruton's tyrosine kinase from Homo sapiens, as disclosed in, e.g., U.S. Pat. No. 6,326,469 (GenBank Accession No. NP_000052).

The term “Bruton's tyrosine kinase homolog,” as used herein, refers to orthologs of Bruton's tyrosine kinase, e.g., the orthologs from mouse (GenBank Accession No. AAB47246), dog (GenBank Accession No. XP_549139.), rat (GenBank Accession No. NP_001007799), chicken (GenBank Accession No. NP_989564), or zebra fish (GenBank Accession No. XP_698117), and fusion proteins of any of the foregoing that exhibit kinase activity towards one or more substrates of Bruton's tyrosine kinase (e.g. a peptide substrate having the amino acid sequence “AVLESEEELYSSARQ”).

The terms “co-administration” or the like, as used herein, are meant to encompass administration of the selected therapeutic agents to a single patient, and are intended to include treatment regimens in which the agents are administered by the same or different route of administration or at the same or different time. In some embodiments, the term “co-administration”or the like, is meant to encompass the administration of the selected therapeutic agents in the same cycle(s). In these embodiments, the selected therapeutic agents may be administered on the same or different days of the cycle(s).

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound of the present invention, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “homologous cysteine,” as used herein refers to a cysteine residue found with in a sequence position that is homologous to that of cysteine 481 of Bruton's tyrosine kinase, as defined herein. For example, cysteine 482 is the homologous cysteine of the rat ortholog of Bruton's tyrosine kinase; cysteine 479 is the homologous cysteine of the chicken ortholog; and cysteine 481 is the homologous cysteine in the zebra fish ortholog. In another example, the homologous cysteine of TXK, a Tec kinase family member related to Bruton's tyrosine, is Cys 350.

The term “identical,” as used herein, refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection.

By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages are used to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75-100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75-100 nucleic acids in length, over a region that is about 50 nucleic acids in length, or, where not specified, across the entire sequence of a polynucleotide sequence.

The terms “inhibits,” “inhibiting” or “inhibitor” of a kinase, as used herein, refer to inhibition of enzymatic phosphotransferase activity.

The term “irreversible inhibitor” as used herein, refers to a compound that, upon contact with a target protein (e.g., a kinase) causes the formation of a new covalent bond with or within the protein, whereby one or more of the target protein's biological activities (e.g., phosphotransferase activity) is diminished or abolished notwithstanding the subsequent presence or absence of the irreversible inhibitor. In contrast, a reversible inhibitor compound upon contact with a target protein does not cause the formation of a new covalent bond with or within the protein and therefore can associate and dissociate from the target protein.

The term “irreversible Btk inhibitor” as used herein, refers to an inhibitor of Btk that can form a covalent bond with an amino acid residue of Btk. In one embodiment, the irreversible inhibitor of Btk can form a covalent bond with a Cys residue of Btk; in particular embodiments, the irreversible inhibitor can form a covalent bond with a Cys 481 residue (or a homolog thereof) of Btk or a cysteine residue in the homologous corresponding position of another tyrosine kinase.

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

As used herein, the term “selective binding compound” refers to a compound that selectively binds to any portion of one or more target proteins.

As used herein, the term “selectively binds” refers to the ability of a selective binding compound to bind to a target protein, such as, for example, Btk, with greater affinity than it binds to a non-target protein. In certain embodiments, specific binding refers to binding to a target with an affinity that is at least 10, 50, 100, 250, 500, 1000 or more times greater than the affinity for a non-target.

As used herein, the term “selective modulator” refers to a compound that selectively modulates a target activity relative to a non-target activity. In certain embodiments, specific modulater refers to modulating a target activity at least 10, 50, 100, 250, 500, 1000 times more than a non-target activity.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “subject” or “patient” as used herein, refers to an animal which is the object of treatment, observation or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “target protein” refers to a molecule or a portion of a protein capable of being bound by a selective binding compound. In certain embodiments, a target protein is Btk.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, the IC₅₀ refers to an amount, concentration or dosage of a particular test compound that achieves a 50% inhibition of a maximal response, such as inhibition of Btk, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The methods described herein include administering to a subject in need a composition containing a therapeutically effective amount of one or more reversible or irreversible Btk inhibitor compounds described herein. Without being bound by theory, the diverse roles played by Btk signaling in various hematopoietic cell functions, e.g., B-cell receptor activation, suggests that small molecule Btk inhibitors are useful for reducing the risk of or treating a variety of diseases affected by or affecting many cell types of the hematopoetic lineage including, e.g., autoimmune diseases, heteroimmune conditions or diseases, inflammatory diseases, cancer (e.g., B-cell proliferative disorders), and thromboembolic disorders. Further, the irreversible Btk inhibitor compounds described herein can be used to inhibit a small subset of other tyrosine kinases that share homology with Btk by having a cysteine residue (including a Cys 481 residue) that can form a covalent bond with the irreversible inhibitor. Thus, a subset of tyrosine kinases other than Btk are also expected to be useful as therapeutic targets in a number of health conditions.

In some embodiments, the compositions and methods described herein can be used to treat an autoimmune disease, which includes, but is not limited to, rheumatoid arthritis, psoriatic arthritis, osteoarthritis, Still's disease, juvenile arthritis, lupus, diabetes, myasthenia gravis, Hashimoto's thyroiditis, Ord's thyroiditis, Graves' disease Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, autoimmune hemolytic anemia, warm autoimmune hemolytic anemia, cold hemolytic anemia, Wegener's granulomatosis, psoriasis, alopecia universalis, Behçet's disease, chronic fatigue, dysautonomia, endometriosis, interstitial cystitis, neuromyotonia, scleroderma, immune-mediated thrombocytopenia, and vulvodynia.

In some embodiments, the compositions and methods described herein can be used to treat heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the compositions and methods described herein can be used to treat ischemia/reperfusion injury, such as ischemia/reperfusion injury caused by transplantation, heart attack, stroke, or the like.

In some embodiments, the compositions and methods described herein can be used to treat an inflammatory disease, which includes, but is not limited to asthma, inflammatory bowel disease, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some embodiments, the compositions and methods described herein can be used to treat a cancer, e.g., B-cell proliferative disorders, which include, but are not limited to diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, and lymphomatoid granulomatosis.

In some embodiments, the methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

In some embodiments, the compositions and methods described herein can be used to treat a solid tumor. In some embodiments, the composition is for use in treatment of a sarcoma or carcinoma. In some embodiments, the composition is for use in treatment of a sarcoma. In some embodiments, the composition is for use in treatment of a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extrasketetal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation: periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the solid tumor is selected from anal cancer; appendix cancer; bile duct cancer (i.e., cholangiocarcinoma); bladder cancer; brain tumor; breast cancer; HER2-amplified breast cancer; cervical cancer; colon cancer; cancer of Unknown Primary (CUP): esophageal cancer; eye cancer; fallopian tube cancer; kidney cancer; renal cell carcinoma; liver cancer; lung cancer; medulloblastoma; melanoma; oral cancer; ovarian cancer; pancreatic cancer; pancreatic ductal cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the carcinoma is breast cancer. In some embodiments, the breast cancer is invasive ductal carcinoma, ductal carcinoma in situ, invasive lobular carcinoma, or lobular carcinoma in situ. In some embodiments, the carcinoma is pancreatic cancer. In some embodiments, the pancreatic cancer is adenocarcinoma, or islet cell carcinoma. In some embodiments, the carcinoma is colorectal cancer. In some embodiments, the colorectal cancer is adenocarcinoma. In some embodiments, the solid tumor is a colon polyp. In some embodiments, the colon polyp is associated with familial adenomatous polyposis. In some embodiments, the carcinoma is bladder cancer. In some embodiments, the bladder cancer is transitional cell bladder cancer, squamous cell bladder cancer, or adenocarcinoma. In some embodiments, the carcinoma is lung cancer. In some embodiments, the lung cancer is a non-small cell lung cancer. In some embodiments, the non-small cell lung cancer is adenocarcinoma, squamous-cell lung carcinoma, or large-cell lung carcinoma. In some embodiments, the non-small cell lung cancer is large cell lung cancer. In some embodiments, the lung cancer is a small cell lung cancer. In some embodiments, the carcinoma is prostate cancer. In some embodiments, the prostate cancer is adenocarcinoma or small cell carcinoma. In some embodiments, the carcinoma is ovarian cancer.

In some embodiments, the ovarian cancer is epithelial ovarian cancer. In some embodiments, the carcinoma is bile duct cancer. In some embodiments, the bile duct cancer is proximal bile duct carcinoma or distal bile duct carcinoma.

In some embodiments, the composition and methods described herein can be used to treat mastocytosis.

In some embodiments, the compositions and methods described herein can be used to treat carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, or Smoldering of indolent multiple myeloma.

In some embodiments, the compositions and methods described herein can be used to treat a central nervous system (CNS) malignancy. In some embodiments, the CNS malignancy is a primary CNS lymphoma. In some embodiments the primary CNS lymphoma is a glioma. In some embodiments the glioma is astrocytomas, ependymomas, oligodendrogliomas. In some embodiments the CNS malignancy is astrocytic tumors such as juvenile pilocytic, subependymal, well differentiated or moderately differentiated anaplastic astrocytoma; anaplastic astrocytoma; glioblastoma multiforme; ependymal tumors such as myxopapillary and well-differentiated ependymoma, anaplastic ependymoma, ependymoblastoma; oligodendroglial tumors including well-differentiated oligodendroglioma and anaplastic oligodendroglioma; mixed tumors such as mixed astrocytoma-ependymoma, mixed astrocytoma-oligodendroglioma, mixed astrocytomaependymoma-oligodendroglioma; or medulloblastoma.

In some embodiments, the compositions and methods described herein can be used to treat hematological malignancies such as, but not limited to, a leukemia, a lymphoma, a myeloma, a non-Hodgkin's lymphoma, a Hodgkin's lymphoma, a T-cell malignancy, or a B-cell malignancy. In some embodiments, the hematological malignancy is a treatment naive hematological malignancy. In some embodiments the hematological malignancy is a relapsed or refractory hematological malignancy.

In some embodiments, the hematologic malignancy is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the T-cell malignancy is a relapsed or refractory T-cell malignancy. In some embodiments, the T-cell malignancy is a treatment naïve T-cell malignancy.

In some embodiments, the hematologic malignancy is a B-cell proliferative disorder. In some embodiments, the cancer is chronic lymphocytic leukemia (CLL), small lymphocytic lymphoma (SLL), high risk CLL, a non-CLL/SLL lymphoma, or prolymphocytic leukemia (PLL). In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL), germinal center diffuse large B-cell lymphoma (GCB DLBCL), and Double-Hit (DH) DLBCL. In some embodiments, ABC-DLBCL is characterized by a CD79B mutation. In some embodiments, ABC-DLBCL is characterized by a CD79A mutation. In some embodiments, the ABC-DLBCL is characterized by a mutation in MyD88, A20, or a combination thereof. In some embodiments, the cancer is acute or chronic myelogenous (or myeloid) leukemia, myelodysplastic syndrome, or acute lymphoblastic leukemia. In some embodiments, the B-cell proliferative disorder is a relapsed and refractory B-cell proliferative disorder. In some embodiments, the B-cell proliferative disorder is a treatment naive B-cell proliferative disorder.

In some embodiments, the compositions and methods described herein can be used to treat a hematological malignancy (including leukemia, peripheral T-cell lymphoma, anaplastic large cell lymphoma, angioimmunoblastic lymphoma, cutaneous T-cell lymphoma, adult T-cell leukemia/lymphoma, blastic NK-cell lymphoma, lymphoblastic lymphoma, NK/T-cell lymphoma, treatment-related T cell lymphoma, T-cell acute lymphoblastic leukemia (T-cell ALL), T-cell polymorphocytic leukemia, or large granular lymphocytic leukemiadiffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma). In an embodiment the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis.

In some embodiments, the compositions and methods described herein can be used to treat fibrosis. In some embodiments, the fibrosis is not associated with graft versus host disease (GVHD). In some embodiments, the fibrosis is not associated with sclerodermatous GVHD, lung chronic GVHD, or liver chronic GVHD. In some embodiments, the fibrosis is of the liver, lung, pancreas, kidney, bone marrow, heart, skin, intestine, or joints. In some embodiments, the fibrosis is of the liver. In some embodiments, the fibrosis is of the lung. In some embodiments, the fibrosis is of the pancreas. In some embodiments, the patient has cirrhosis, chronic pancreatitis, or cystic fibrosis.

In some embodiments, the compositions and methods described herein can be used to treat thromboembolic disorders, which include, but are not limited to myocardial infarct, angina pectoris (including unstable angina), reocclusions or restenoses after angioplasty or aortocoronary bypass, stroke, transitory ischemia, peripheral arterial occlusive disorders, pulmonary embolisms, and deep venous thromboses.

Symptoms, diagnostic tests, and prognostic tests for each of the above-mentioned conditions are known in the art. See, e.g., Harrison's Principles of Internal Medicine©,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

A number of animal models of are useful for establishing a range of therapeutically effective doses of reversible or irreversible Btk inhibitor compounds for treating any of the foregoing diseases.

For example, dosing of reversible or irreversible Btk inhibitor compounds for treating an autoimmune disease can be assessed in a mouse model of rheumatoid arthritis. In this model, arthritis is induced in Balb/c mice by administering anti-collagen antibodies and lipopolysaccharide. See Nandakumar et al. (2003), Am. J. Pathol 163:1827-1837.

In another example, dosing of reversible or irreversible Btk inhibitors for the treatment of B-cell proliferative disorders can be examined in, e.g., a human-to-mouse xenograft model in which human B-cell lymphoma cells (e.g. Ramos cells) are implanted into immunodefficient mice (e.g., “nude” mice) as described in, e.g., Pagel et al. (2005), Clin Cancer Res 11(13):4857-4866.

Animal models for treatment of thromboembolic disorders are also known.

The therapeutic efficacy of the compound for one of the foregoing diseases can be optimized during a course of treatment. For example, a subject being treated can undergo a diagnostic evaluation to correlate the relief of disease symptoms or pathologies to inhibition of in vivo Btk activity achieved by administering a given dose of an irreversible Btk inhibitor. Cellular assays known in the art can be used to determine in vivo activity of Btk in the presence or absence of an irreversible Btk inhibitor. For example, since activated Btk is phosphorylated at tyrosine 223 (Y223) and tyrosine 551 (Y551), phospho-specific immunocytochemical staining of P-Y223 or P-Y551-positive cells can be used to detect or quantify activation of Bkt in a population of cells (e.g., by FACS analysis of stained vs unstained cells). See, e.g., Nisitani et al. (1999), Proc. Natl. Acad. Sci, USA 96:2221-2226. Thus, the amount of the Btk inhibitor compound that is administered to a subject can be increased or decreased as needed so as to maintain a level of Btk inhibition optimal for treating the subject's disease state.

### Compounds

In the following description of Btk inhibitory compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. In addition, nucleic acid and amino acid sequences for Btk (e.g., human Btk) are known in the art as disclosed in, e.g., U.S. Pat. No. 6,326,469. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

The Btk inhibitor compounds described herein are selective for Btk and kinases having a cysteine residue in an amino acid sequence position of the tyrosine kinase that is homologous to the amino acid sequence position of cysteine 481 in Btk. Inhibitor compounds described herein include a Michael acceptor moiety.

Generally, a reversible or irreversible inhibitor compound of Btk used in the methods described herein is identified or characterized in an in vitro assay, e.g., an acellular biochemical assay or a cellular functional assay. Such assays are useful to determine an in vitro IC₅₀ for a reversible or irreversible Btk inhibitor compound.

For example, an acellular kinase assay can be used to determine Btk activity after incubation of the kinase in the absence or presence of a range of concentrations of a candidate irreversible Btk inhibitor compound. If the candidate compound is in fact an irreversible Btk inhibitor, Btk kinase activity will not be recovered by repeat washing with inhibitor-free medium. See, e.g., J. B. Smaill, et al. (1999), J. Med. Chem. 42(10): 1803-1815. Further, covalent complex formation between Btk and a candidate irreversible Btk inhibitor is a useful indicator of irreversible inhibition of Btk that can be readily determined by a number of methods known in the art (e.g., mass spectrometry). For example, some irreversible Btk-inhibitor compounds can form a covalent bond with Cys 481 of Btk (e.g., via a Michael reaction).

Cellular functional assays for Btk inhibition include measuring one or more cellular endpoints in response to stimulating a Btk-mediated pathway in a cell line (e.g., BCR activation in Ramos cells) in the absence or presence of a range of concentrations of a candidate irreversible Btk inhibitor compound. Useful endpoints for determining a response to BCR activation include, e.g., autophosphorylation of Btk, phosphorylation of a Btk target protein (e.g., PLC-γ), and cytoplasmic calcium flux.

High throughput assays for many acellular biochemical assays (e.g., kinase assays) and cellular functional assays (e.g., calcium flux) are well known to those of ordinary skill in the art.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. Automated systems thereby allow the identification and characterization of a large number of reversible or irreversible Btk compounds without undue effort.

Reversible or irreversible Btk inhibitor compounds can be used for the manufacture of a medicament for treating any of the foregoing conditions (e.g., autoimmune diseases, inflammatory diseases, allergy disorders, B-cell proliferative disorders, or thromboembolic disorders).

In some embodiments, the reversible or irreversible Btk inhibitor compound used for the methods described herein inhibits Btk or a Btk homolog kinase activity with an in vitro IC₅₀ of less than about 10 μM, less than about 1 μM, less than about 0.5 μM, less than about 0.4 μM, less than about 0.3 μM, less than about 0.1 μM, less than about 0.08 μM, less than about 0.06 μM, less than about 0.05 μM, less than about 0.04 μM, less than about 0.03 μM, less than about 0.02 μM, less than about 0.01 μM, less than about 0.008 μM, less than about 0.006 μM, less than about 0.005 μM, less than about 0.004 μM, less than about 0.003 μM, less than about 0.002 μM, less than about 0.001 μM, less than about 0.00099 μM, less than about 0.00098 μM, less than about 0.00097 μM, less than about 0.00096 μM, less than about 0.00095 μM, less than about 0.00094 μM, less than about 0.00093 μM, less than about 0.00092, or less than about 0.00090 μM.

In one embodiment, the Btk inhibitor compound selectively inhibits an activated form of its target tyrosine kinase (e.g., a phosphorylated form of the tyrosine kinase). For example, activated Btk is transphosphorylated at tyrosine 551. Thus, in these embodiments the Btk inhibitor inhibits the target kinase in cells only once the target kinase is activated by the signaling events.

Described herein are compounds of the present invention. Also described herein are pharmaceutically acceptable salts, pharmaceutically acceptable solvates, pharmaceutically active metabolites, and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided. In some embodiments, when compounds disclosed herein contain an oxidizable nitrogen atom, the nitrogen atom can be converted to an N-oxide by methods well known in the art. In certain embodiments, isomers and chemically protected forms of compounds having a structure represented by any one of the Formulas described herein are also provided.

In some embodiments, the present invention is a compound having the structure of Formula (A-I):

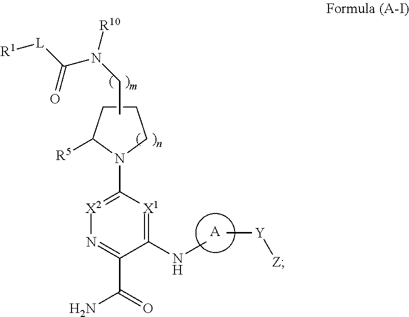

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a single bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3;
or a pharmaceutically acceptable solvate, pharmaceutically acceptable salt, and/or pharmaceutically acceptable prodrug thereof.

In some embodiments of Formula (A-I), R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl fused with a substituted or unsubstituted phenyl ring.

In some embodiments of Formula (A-I), m is 1 and R¹ is substituted or unsubstituted C₁-C₄alkyl, substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl.

In some embodiments of Formula (A-I), R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

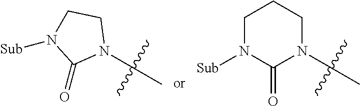

(wherein Sub represents H or a substituent).

In some embodiments of Formula (A-I), X² is N. In some embodiments of Formula (A-I), m is 0, R¹ is substituted or unsubstituted C₂-C₄alkenyl, X¹ is N, and X² is N. In some embodiments, X² is C(R²), wherein R² is H, —CN, halogen, —OH, substituted or unsubstituted C₁-C₄alkoxy, substituted or unsubstituted methyl, propyl, isopropyl, or C₄alkyl, substituted or unsubstituted C₃-C₆cycloalkyl, substituted or unsubstituted C₂-C₆heterocycloalkyl, or —N(R³)₂.

In some embodiments, m is 0, R¹ is substituted or unsubstituted C₂-C₄alkenyl, X¹ is N, and X² is C(R²), wherein R² is H, —CN, halogen, —OH, substituted or unsubstituted C₁-C₄alkoxy, substituted or unsubstituted methyl, propyl, isopropyl, or C₄alkyl, substituted or unsubstituted C₃-C₆cycloalkyl, substituted or unsubstituted C₂-C₆heterocycloalkyl, or —N(R³)₂. In some embodiments, X² is C(R²), wherein R² is H, —CN, halogen, —OH, substituted or unsubstituted C₁-C₄alkoxy, substituted C₁-C₄alkyl, substituted or unsubstituted C₃-C₆cycloalkyl, substituted or unsubstituted C₂-C₆heterocycloalkyl, or —N(R³)₂.

In some embodiments of Formula (A-I), m is 1.

In some embodiments of Formula (A-I), L is NR¹¹.

In some embodiments of Formula (A-I), A is substituted or unsubstituted C₁-C₁₂heteroaryl. In some embodiments of Formula (A-I), m is 0 and A is substituted or unsubstituted C₁-C₁₂heteroaryl. In some embodiments of Formula (A-I), R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl. In some embodiments of Formula (A-I), m is 0 and R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₂aryl, or substituted or unsubstituted C₁-C₁₂heteroaryl.

In some embodiments of Formula (A-I), n is 0. In some embodiments, n is 0 and m is 1.

In some embodiments of Formula (A-I), n is 0, R¹ is substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl; and A is substituted or unsubstituted C₁-C₁₂heteroaryl.

In some embodiments of Formula (A-I):
n is 0;
m is 1;
L is NR¹¹;
A is substituted or unsubstituted C₁-C₁₂heteroaryl;
Y is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₆-C₁₂aryl, or substituted or unsubstituted C₁-C₁₂heteroaryl; or R¹ is substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl; and
A is substituted or unsubstituted C₁-C₁₂heteroaryl.

In some embodiments of Formula (A-I), the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-yl)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperdin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrabydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrabydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide; and
(R)-5-(3-(3-(tetrahydro-2H-pyran-4-yl)ureido)piperidin-1-yl)-3-(tetrahydro-2H-pyran-4-ylamino)pyrazine-2-carboxamide.

In some embodiments, the present invention is a compound having the structure of Formula (A-I):

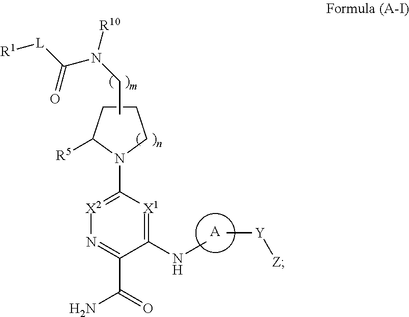

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a single bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

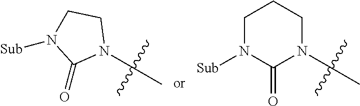

(wherein Sub represents H or a substituent); when m is 1, R¹ may also be substituted or unsubstituted C₁-C₄alkyl;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3;
or a pharmaceutically acceptable solvate, pharmaceutically acceptable salt, and/or pharmaceutically acceptable prodrug thereof;
provided that:
(1) when m is 0, R¹ is substituted or unsubstituted C₂-C₄alkenyl, and X¹ is N, then X² is other than C(Et);
(2) when m is 0, then -A-Y—Z is other than

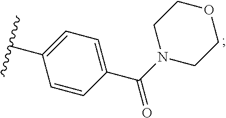 [SMILES: *c1ccc(C(=O)N2CCOCC2)cc1 ; ";"]

(3) when m is 0 and L is a single bond, then R¹ is other than

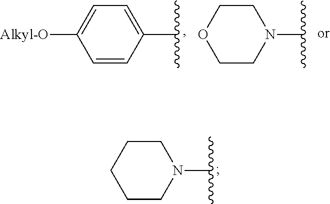

and
(4) the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-y)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperidin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrahydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrahydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide; and
(R)-5-(3-(3-(tetrahydro-2H-pyran-4-yl)ureido)piperidin-1-yl)-3-(tetrahydro-2H-pyran-4-ylamino)pyrazine-2-carboxamide.

In some embodiments, R² is H.

In some embodiments, the present invention is a compound having the structure of Formula (A-VII):

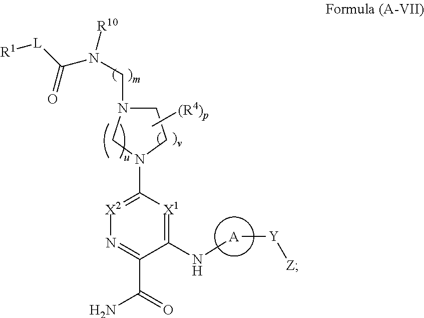

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a single bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is optionally present and when present is NR¹¹;
R¹ is substituted or unsubstituted C₁-C₄alkyl, substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— that separates them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

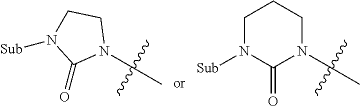

(wherein Sub represents H or a substituent);
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3;
p is 0, 1, 2 or 3;
u is 1, 2 or 3; and
v is 0, 1, 2 or 3;
or a pharmaceutically acceptable solvate, pharmaceutically acceptable salt, and/or pharmaceutically acceptable prodrug thereof.

In some embodiments, R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl or a substituted or unsubstituted C₂-C₇heterocycloalkyl fused with a substituted or unsubstituted phenyl ring.

In some embodiments, m is 1 and R¹ is substituted or unsubstituted C₁-C₄alkyl, substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— moiety between them form a substituted or unsubstituted C₁-C₁₂heteroaryl.

In some embodiments, R¹ and R¹⁰ together with the -L-C(O)—N— moiety between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than

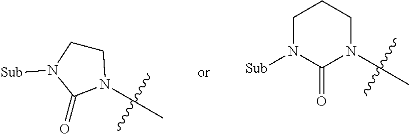

(wherein Sub represents H or a substituent).

In some embodiments, X² is N. In some embodiments, m is 0, R¹ is substituted or unsubstituted C₂-C₄alkenyl, X¹ is N, and X² is N. In some embodiments, X² is C(R²), wherein R² is H, —CN, halogen, —OH, substituted or unsubstituted C₁-C₄alkoxy, substituted or unsubstituted methyl, propyl, isopropyl, or C₄alkyl, substituted or unsubstituted C₃-C₆cycloalkyl, substituted or unsubstituted C₂-C₆heterocycloalkyl, or —N(R³)₂. In some embodiments, m is 0, R¹ is substituted or unsubstituted C₂-C₄alkenyl, X¹ is N, and X² is C(R²), wherein R² is H, —CN, halogen, —OH, substituted or unsubstituted C₁-C₄alkoxy, substituted or unsubstituted methyl, propyl, isopropyl, or C₄alkyl, substituted or unsubstituted C₃-C₆cycloalkyl, substituted or unsubstituted C₂-C₆heterocycloalkyl, or —N(R³)₂. In some embodiments, X² is C(R²), wherein R² is H, —CN, halogen, —OH, substituted or unsubstituted C₁-C₄alkoxy, substituted C₁-C₄alkyl, substituted or unsubstituted C₃-C₆cycloalkyl, substituted or unsubstituted C₂-C₆heterocycloalkyl, or —N(R³)₂.

In some embodiments, m is 1.

In some embodiments, L is NR¹¹.

In some embodiments, A is substituted or unsubstituted C₁-C₁₂heteroaryl. In some embodiments, m is 0 and A is substituted or unsubstituted C₁-C₁₂heteroaryl. In some embodiments, R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl. In some embodiments, m is 0 and R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁aryl, or substituted or unsubstituted C₁-C₁₂heteroaryl.

In some embodiments:
m is 1;
X² is N or C(R²), wherein R² is H, —CN, halogen, —OH, substituted or unsubstituted C₁-C₄alkoxy, substituted or unsubstituted methyl, propyl, isopropyl, or C₄alkyl, substituted or unsubstituted C₃-C₆cycloalkyl, substituted or unsubstituted C₂-C₆heterocycloalkyl, or —N(R³)₂; L is NR¹¹;
A is substituted or unsubstituted C₁-C₁₂heteroaryl; Y is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₆-C₁₂aryl, or substituted or unsubstituted C₁-C₁₂heteroaryl; and
R¹ is substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl; and A is substituted or unsubstituted C₁-C₂₁heteroaryl.

In some embodiments, when m is 0, R¹ is substituted or unsubstituted C₁-C₄alkyl or substituted or unsubstituted C₂-C₄alkenyl, and X¹ is N, then X² is other than C(Et).

In some embodiments, when m is 0, then -A-Y—Z is other than

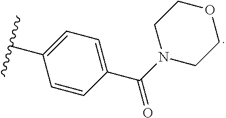 [SMILES: *c1ccc(C(=O)N2CCOCC2)cc1]

In some embodiments, when m is 0 and L is a single bond, then R¹ is other than

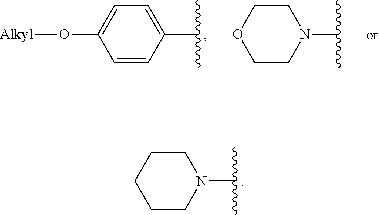

In some embodiments, u is 1. In some embodiments, u is 2. In some embodiments, u is 3.

In some embodiments, v is 0. In some embodiments, v is 1. In some embodiments, v is 2. In some embodiments, v is 3.

In one aspect, provided herein is a compound of Formula (A-IA) having the structure:

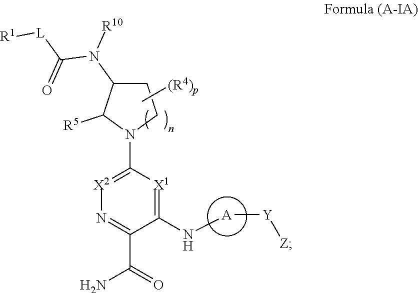

wherein A, L, X¹, X², Y, Z, R¹, R⁴, R⁵, R¹⁰, n and p are as defined herein;
or a pharmaceutically acceptable solvate, pharmaceutically acceptable salt, and/or pharmaceutically acceptable prodrug thereof.

In one aspect, provided herein is a compound of Formula (A-IB) having the structure:

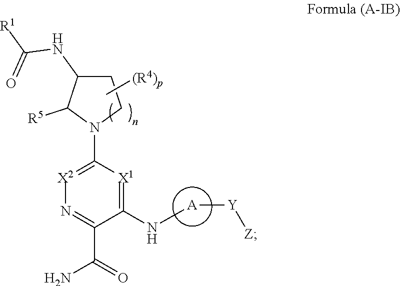

wherein A, X¹, X², Y, Z, R¹, R⁴, R⁵, n and p are as defined herein;
or a pharmaceutically acceptable solvate, pharmaceutically acceptable salt, and/or pharmaceutically acceptable prodrug thereof.

In one aspect, provided herein is a compound of Formula (A-IC) or (ID) having the structure:

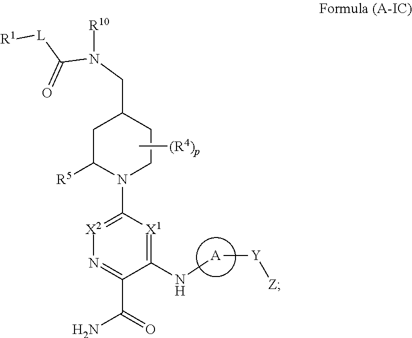

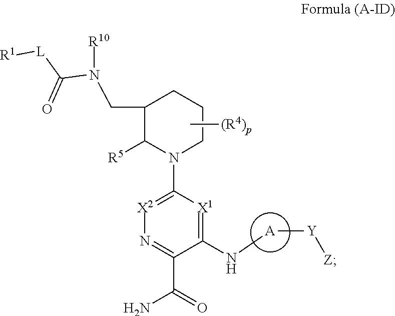

wherein A, L, X¹, X², Y, Z, R¹, R⁴, R⁵, R¹⁰ and p are as defined herein;
or a pharmaceutically acceptable solvate, pharmaceutically acceptable salt, and/or pharmaceutically acceptable prodrug thereof.

In one aspect, provided herein is a compound of Formula (A-IE) having the structure:

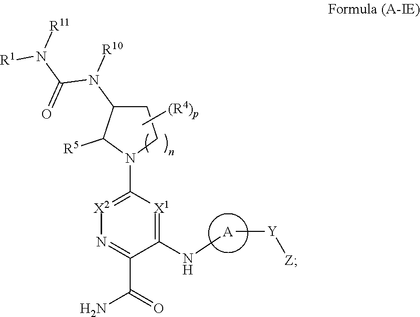

wherein A, X¹, X², Y, Z, R¹, R⁴, R⁵, R¹⁰, R¹¹, n and p are as defined herein;
or a pharmaceutically acceptable solvate, pharmaceutically acceptable salt, and/or pharmaceutically acceptable prodrug thereof.

In one aspect, provided herein is a compound of Formula (A-IF) having the structure:

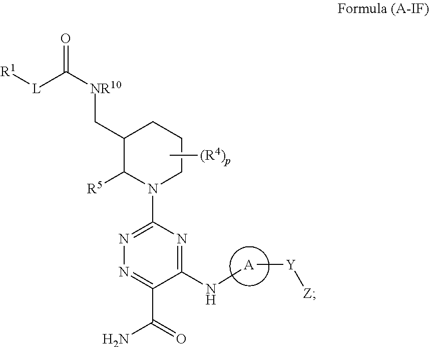

wherein A, L, Y, Z, R¹, R⁴, R⁵, R¹⁰ and p are as defined herein;
or a pharmaceutically acceptable solvate, pharmaceutically acceptable salt, and/or pharmaceutically acceptable prodrug thereof.

In one aspect, provided herein is a compound of Formula (A-IG) having the structure:

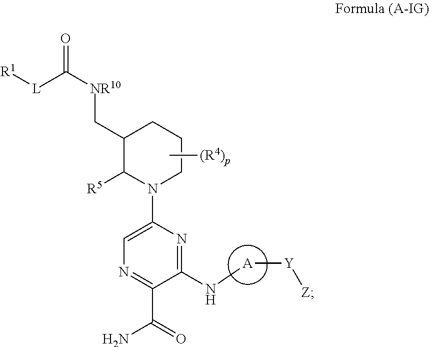

wherein A, L, Y, Z, R¹, R⁴, R⁵, R¹⁰ and p are as defined herein;
or a pharmaceutically acceptable solvate, pharmaceutically acceptable salt, and/or pharmaceutically acceptable prodrug thereof.

In one aspect, provided herein is a compound of Formula (A-IH) having the structure:

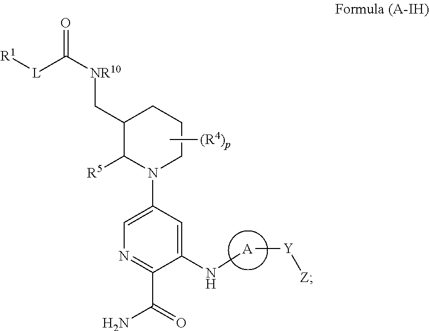

wherein A, L, Y, Z, R¹, R⁴, R⁵, R¹⁰ and p are as defined herein;
or a pharmaceutically acceptable solvate, pharmaceutically acceptable salt, and/or pharmaceutically acceptable prodrug thereof.

In some embodiments, provided herein are are compounds having the structure of Formula (B-I):

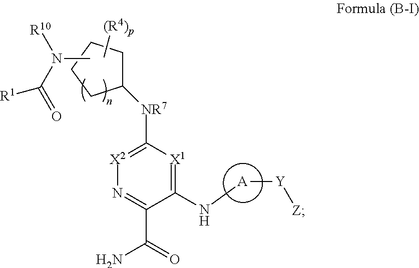

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is optionally present and when present is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is optionally present and when present is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R¹⁰ together with —C(O)—N— form a substituted or unsubstituted C₁-C₁₂heteroaryl or substituted or unsubstituted C₂-C₇heterocycloalkyl optionally fused with a substituted or unsubstituted phenyl ring;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is substituted or unsubstituted C₁-C₆alkyl, substituted or unsubstituted C₂-C₄alkenyl, substituted or unsubstituted C₂-C₄alkynyl, substituted or unsubstituted C₆—C cycloalkyl, substituted or unsubstituted C₂-C₇heterocycloalkyl, substituted or unsubstituted C₆-C₁₂aryl, or substituted or unsubstituted C₁-C₁₂heteroaryl;
R⁷ is H, or substituted or unsubstituted C₁-C₄alkyl;
R¹⁰ and R¹¹ are independently H, or substituted or unsubstituted C₁-C₄alkyl; or R¹⁰ and R¹¹ connect to form a C₁-C₄alkylene;
n is 0, 1, 2 or 3; and
p is 0, 1, 2 or 3;
or a pharmaceutically acceptable solvate, pharmaceutically acceptable salt, or pharmaceutically acceptable prodrug thereof.

In some embodiments of Formula (B-I), R¹ is substituted or unsubstituted C₂-C₄alkenyl, A is substituted or unsubstituted phenyl, R⁷ is H, the group

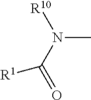

and the group

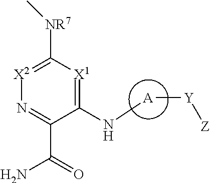

are attached to the same carbon atom or attached to carbon atoms that are adjacent to each other, X¹ is N, and X² is N or C(R²), wherein R² is —CN, halogen, —OH, substituted or unsubstituted C₁-C₄alkoxy, substituted or unsubstituted methyl, substituted or unsubstituted C₃-C₆cycloalkyl, substituted or unsubstituted C₂-C₆heterocycloalkyl, or —N(R³)₂.

In some embodiments of Formula (B-I):
R¹ is substituted or unsubstituted C₁-C₄alkyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R¹⁰ together with —C(O)—N— form a substituted or unsubstituted C₁-C₁₂heteroaryl or substituted or unsubstituted C₂-C₇heterocycloalkyl optionally fused with a substituted or unsubstituted phenyl ring;
A is substituted or unsubstituted C₁-C₁₂heteroaryl;
the group

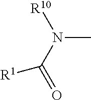

and the group

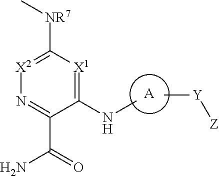

are not attached to the same carbon atom or to carbon atoms that are adjacent to each other;
X¹ is C(R²); and
X² is N or C(R²), wherein R² is —CN, halogen, —OH, substituted or unsubstituted C₁-C₄alkoxy, substituted or unsubstituted methyl, substituted or unsubstituted C₃-C₆cycloalkyl, substituted or unsubstituted C₂-C₆heterocycloalkyl, or —N(R³)₂.

In some embodiments of Formula (B-I), the compound is other than 5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide.

In some embodiments, provided herein are compounds having the structure of Formula (B-I):

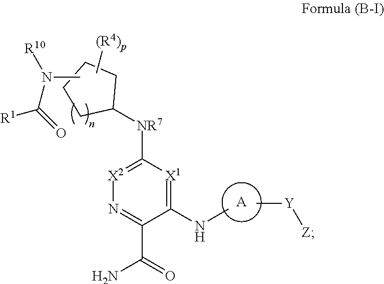

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is optionally present and when present is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is optionally present and when present is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R¹⁰ together with —C(O)—N— form a substituted or unsubstituted C₁-C₁₂heteroaryl or substituted or unsubstituted C₂-C₇heterocycloalkyl optionally fused with a substituted or unsubstituted phenyl ring;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is substituted or unsubstituted C₁-C₆alkyl, substituted or unsubstituted C₂-C₄alkenyl, substituted or unsubstituted C₂-C₄alkynyl, substituted or unsubstituted C₆-C₇cycloalkyl, substituted or unsubstituted C₂-C₇heterocycloalkyl, substituted or unsubstituted C₆-C₁₂aryl, or substituted or unsubstituted C₁-C₁₂heteroaryl;
R⁷ is H, or substituted or unsubstituted C₁-C₄alkyl;
R¹⁰ and R¹¹ are independently H, or substituted or unsubstituted C₁-C₄alkyl; or R¹⁰ and R¹¹ connect to form a C₁-C₄alkylene;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when R¹ is substituted or unsubstituted C₂-C₄alkenyl, A is substituted or unsubstituted phenyl, R⁷ is H, the group

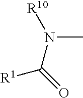

and the group

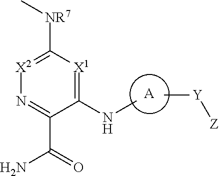

are attached to the same carbon atom or attached to carbon atoms that are adjacent to each other, and X¹ is N, then X² is other than CH or C(Et); and
(2) the compound is other than 5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide.

In some embodiments, the group

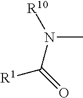

and the group

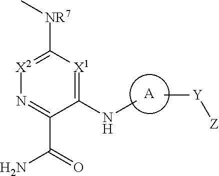

are attached to the same carbon atom or attached to carbon atoms that are adjacent to each other, and X¹ and X² are both N.

In some embodiments, the group

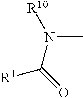

and the group

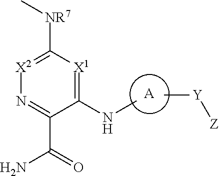

are not attached to the same carbon atom or attached to carbon atoms that are adjacent to each other.

In some embodiments, the present invention provides compounds having the structure of Formula (B-IA):

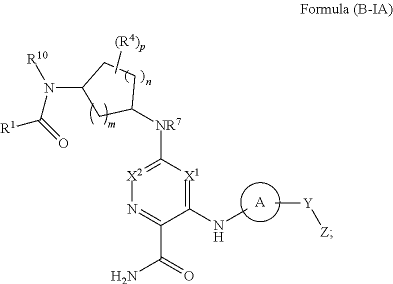

or a pharmaceutically acceptable solvate, pharmaceutically acceptable salt, or pharmaceutically acceptable prodrug thereof, wherein A, X¹, X², Y, Z, R¹, R⁴, R⁷, R¹⁰, n and p are as defined herein, and m is 1, 2, or 3.

In some embodiments, the present invention provides compounds having the structure of Formula (B-IB):

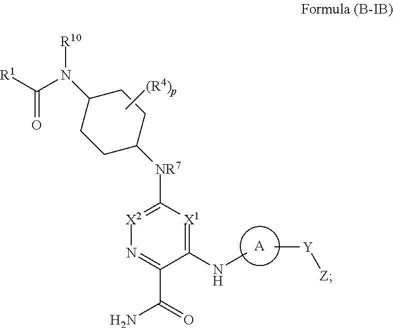

wherein A, X¹, X², Y, Z, R¹, R⁴, R⁷, R¹⁰, and p are as defined herein;
or a pharmaceutically acceptable solvate, pharmaceutically acceptable salt, or pharmaceutically acceptable prodrug thereof.

In some embodiments, the present invention provides a compound of Formula (B-I), (B-II), (B-IA) or (B-IB) wherein R¹ is substituted or unsubstituted C₆-C₁₂aryl, or substituted or unsubstituted C₁-C₁₂heteroaryl. In another embodiment is a compound of Formula (B-I), (B-II), (B-IA) or (B-IB) wherein R¹ is substituted or unsubstituted phenyl. In some embodiments, the compound is a compound of Formula (B-I), (B-II), (B-IA) or (B-IB) wherein R¹ is substituted or unsubstituted pyridyl. In some embodiments, the compound is a compound of Formula (B-I), (B-II), (B-IA) or (B-IB) wherein R¹ is substituted or unsubstituted pyrimidinyl. In some embodiments, the compound is a compound of Formula (B-I), (B-II), (B-IA) or (B-IB) wherein R¹ is substituted or unsubstituted indolyl. In some embodiments, the compound is a compound of Formula (B-I), (B-II), (B-IA) or (B-IB) wherein R¹ is substituted or unsubstituted benzimidazolyl. In some embodiments, the compound is a compound of Formula (B-I), (B-II), (B-IA) or (B-IB) wherein R¹ is substituted or unsubstituted benzofuranyl. In some embodiments, the compound is a compound of Formula (B-I), (B-II), (B-IA) or (B-IB) wherein R¹ is substituted isoindolinyl. In some embodiments, the compound is a compound of Formula (B-I), (B-II), (B-IA) or (B-IB) wherein R¹ is unsubstituted isoindolinyl.

In some embodiments, the present invention provides compounds having the structure of Formula (C-I):

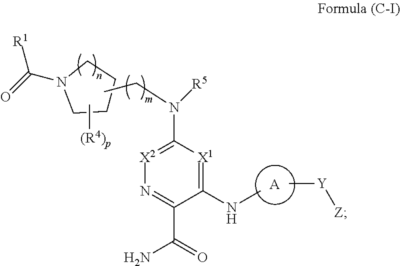

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is optionally present and when present is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is optionally present and when present is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, or substituted or unsubstituted C₁-C₄alkyl;
R⁷ is independently substituted or unsubstituted C₁-C₄alkyl, substituted or unsubstituted C₂-C₄alkenyl, substituted or unsubstituted C₂-C₄alkynyl, substituted or unsubstituted C₃-C₆cycloalkyl, substituted or unsubstituted C₂-C₄heterocycloalkyl, substituted or unsubstituted C₆-C₁₂aryl, or substituted or unsubstituted C₁-C₁₂heteroaryl;
R¹⁰ is H, or substituted or unsubstituted C₁-C₄alkyl;
m is 0 or 1;
n is 0, 1, 2 or 3; and
p is 0, 1, 2 or 3;
or a pharmaceutically acceptable solvate, pharmaceutically acceptable salt, or pharmaceutically acceptable prodrug thereof.

In some embodiments of Formula (C-I), m is 1.

In some embodiments of Formula (C-I), A is substituted or unsubstituted C₁-C₁₂heteroaryl. In some embodiments of Formula (C-I), m is 0 and A is substituted or unsubstituted C₁-C₁₂heteroaryl.

In some embodiments of Formula (C-I), R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl.

In some embodiments, m is 0 and R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl. In some embodiments, A is quinolinyl, m is 0, X¹ is N, X² is CH, and R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl.

In some embodiments of Formula (C-I), X¹ is N and X² is CH or N. In some embodiments of Formula (C-I), X1 is C(R²).

In some embodiments of Formula (C-I), the compound is not
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide; or
(R)-3-(5-carbamoyl-6-(3-methylisothiazol-5-ylamino)pyrazin-2-ylamino)-N,N-dimethylazepane-1-carboxamide.

In some embodiments, the present invention provides compounds having the structure of Formula (C-I):

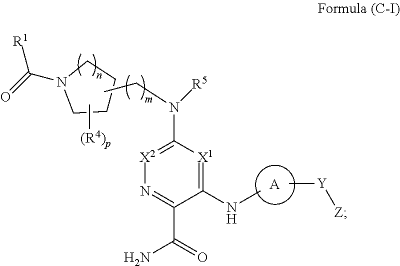

wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is optionally present and when present is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is optionally present and when present is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, or substituted or unsubstituted C₁-C₄alkyl;
R¹ is independently substituted or unsubstituted C₁-C₄alkyl, substituted or unsubstituted C₂-C₄alkenyl, substituted or unsubstituted C₂-C₄alkynyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₂heteroaryl;
R¹⁰ is H, or substituted or unsubstituted C₁-C₄alkyl;
m is 0 or 1;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when m is 0, then -A-Y—Z is not

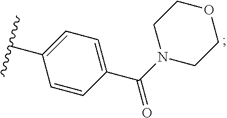 [SMILES: *c1ccc(C(=O)N2CCOCC2)cc1]

(2) when A is quinolinyl, m is 0, X¹ is N and X² is CH, then R¹ is other than Me;
(3) when R¹ is substituted or unsubstituted C₁-C₄alkyl or substituted or unsubstituted C₂-C₄alkenyl, m is 0, and X¹ is N, then X² is CH or N; and
(4) the compound is not
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide; or
(R)-3-(5-carbamoyl-6-(3-methylisothiazol-5-ylamino)pyrazin-2-ylamino)-N,N-dimethylazepane-1-carboxamide;
or a pharmaceutically acceptable solvate, pharmaceutically acceptable salt, or pharmaceutically acceptable prodrug thereof.

In another embodiment are compounds having the structure of Formula (C-IA):

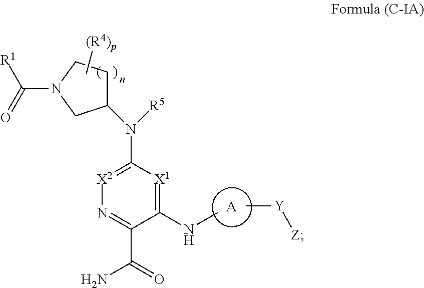

wherein:
A, X¹, X², Y, Z, R¹, R⁴, R⁵, n and p are as defined herein;
or a pharmaceutically acceptable solvate, pharmaceutically acceptable salt, or pharmaceutically acceptable prodrug thereof.

In another embodiment are compounds having the structure of Formula (C-IB):

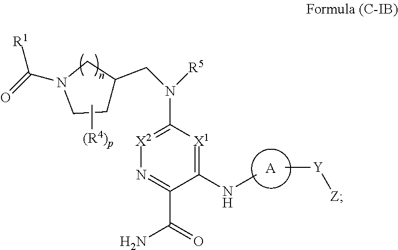

wherein:
A, X¹, X², Y, Z, R¹, R⁴, R⁵, n and p are as defined herein;
or a pharmaceutically acceptable solvate, pharmaceutically acceptable salt, or pharmaceutically acceptable prodrug thereof.

In some embodiments, the present invention provides a compound of Formula (A-I), (A-II), (A-IA)-(A-IH) or (A-VII), or Formula (B-I), (B-II), (B-IA) or (B-IB), or Formula (C-I), (C-IA) or (C-IB) wherein R¹ is substituted or unsubstituted C₂-C₄alkenyl, or substituted or unsubstituted C₂-C₄alkynyl. In some embodiments, R¹ is unsubstituted C₂-C₄alkenyl or unsubstituted C₂-C₄alkynyl. In some embodiments, R¹ is C₂-C₄alkenyl substituted with OR¹⁷ or NR¹⁷R¹⁸, wherein R¹ and R¹⁸ are independently H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R¹ is C₂-C₄alkynyl substituted with OR¹⁷ or NR¹⁷R¹⁸.

In some embodiments, the present invention provides a compound of Formula (C-I), (C-IA) or (C-IB) wherein R¹ is substituted or unsubstituted C₁-C₄alkyl. In some embodiments, R¹ is C₂-C₄alkenyl substituted with OR¹⁷ or NR¹⁷R¹⁸, wherein R¹⁷ and R¹⁸ are as defined herein. In some embodiments, R¹ is C₂-C₄alkynyl substituted with OR¹⁷ or NR¹⁷R¹⁸, wherein R¹⁷ and R¹⁸ are as defined herein.

In some embodiments, the group R¹ is

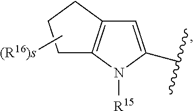

wherein R¹⁵ is C₁-C₄alkyl, such as methyl, or halo, such as F, Cl, or Br; each R¹⁶ is independently H or C₁-C₃alkyl, such as methyl; and s is 0, 1 or 2.

In some embodiments, the present invention provides a compound of Formula (A-I), (A-II), (A-IA)-(A-IH) or (A-VII), or Formula (B-I), (B-II), (B-IA) or (B-IB), or, Formula (C-I), (C-IA) or (C-IB) wherein R¹ is selected from:

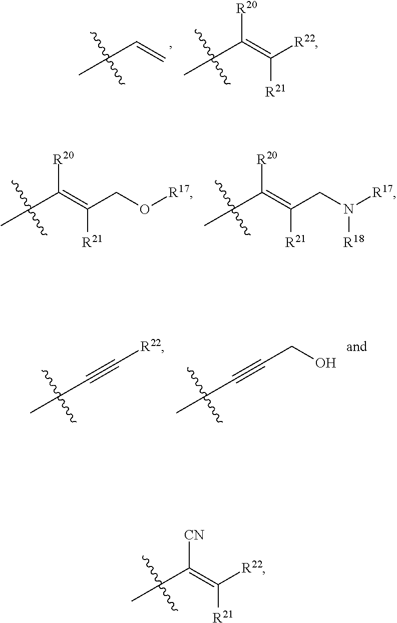

wherein R¹⁷, R¹⁸, R²⁰, R²¹ and R²² are as defined herein.

In some embodiments, R²⁰ and R²¹ are H, R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, all of R²⁰, R²¹ and R²² are H. In some embodiments, R²⁰ and R²¹ together form a bond and R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R²⁰ is CN. In some embodiments, R²⁰ is halo, such as F or Cl.

In some embodiments, the present invention provides a compound of Formula (A-I), (A-II), (A-IA)-(A-IH) or (A-VII), or Formula (B-I), (B-II), (B-IA) or (B-IB), or Formula (C-I), (C-IA) or (C-IB) wherein R¹ is selected from:

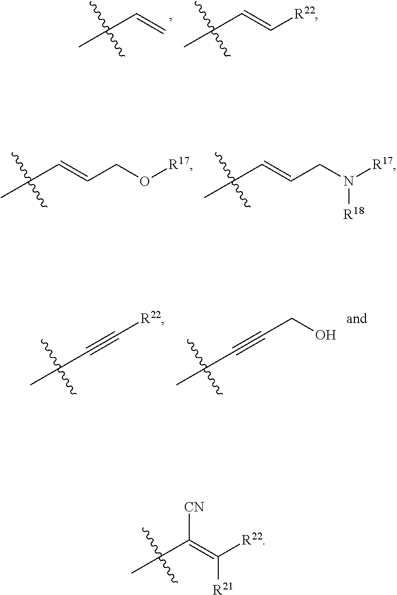

In some embodiments, the present invention provides a compound of Formula (A-I), (A-II), (A-IA)-(A-IH) or (A-VII), or Formula (B-I), (B-II), (B-IA) or (B-IB), or Formula (C-I), (C-IA) or (C-IB) wherein R¹ is selected from CN.

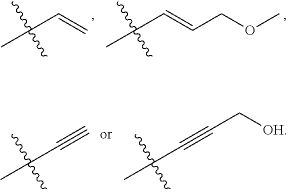

In some embodiments, the present invention provides a compound of Formula (B-I), (B-II), (B-IA) or (B-IB), or Formula (C-I), (C-IA) or (C-IB) wherein R¹ is NR⁵R¹¹. In some embodiments, the compound is a compound of Formula (B-I), (B-II), (B-IA) or (B-IB), or Formula (C-I), (C-IA) or (C-IB) wherein R¹ is N(CH₃)₂. In some embodiments, R⁵ is substituted or unsubstituted C₁-C₆alkyl. In some embodiments, R⁵ is substituted or unsubstituted C₆-C₇cycloalkyl. In some embodiments, R⁵ is substituted or unsubstituted C₂-C₇heterocycloalkyl. In some embodiments, R⁵ is substituted or unsubstituted C₆-C₁₂aryl. In some embodiments, R⁵ is substituted or unsubstituted C₁-C₁₂heteroaryl. In some embodiments, R^{S} is substituted or unsubstituted C₂-C₄alkenyl, or substituted or unsubstituted C₂-C₄alkynyl. In some embodiments, R⁵ is unsubstituted C₂-C₄alkenyl or unsubstituted C₂-C₄alkynyl. In some embodiments, R⁵ is C₂-C₄alkenyl substituted with OR¹⁷ or NR¹⁷R¹⁸, wherein R¹⁷ and R¹⁸ are as defined herein.

In some embodiments, R⁵ is selected from

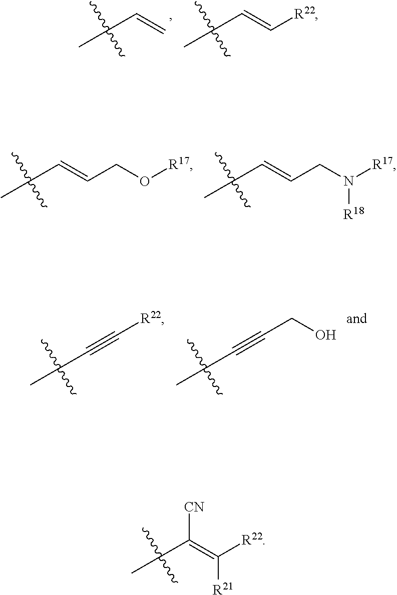

In some embodiments, R⁵ is selected from

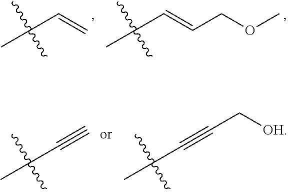

In some embodiments, the present invention provides a compound of Formula (B-I), (B-II), (B-IA) or (B-IB) wherein R¹⁰ is hydrogen. In some embodiments, the compound is a compound of Formula (B-I), (B-II), (B-IA) or (B-IB) wherein R¹⁰ is substituted or unsubstituted C₁-C₄alkyl, such as methyl or ethyl. In some embodiments, the compound is a compound of Formula (B-I), (B-II), (B-IA) or (B-IB) wherein R¹¹ is hydrogen. In some embodiments, the compound is a compound of Formula (B-I), (B-II), (B-IA) or (B-IB) wherein R¹¹ is substituted or unsubstituted C₁-C₄alkyl. In some embodiments, the compound is a compound of Formula (B-I), (B-II), (B-IA) or (B-IB) wherein R¹⁰ and R¹¹ connect to form a C₁-C₄alkylene. In some embodiments, the compound is a compound of Formula (B-I), (B-II), (B-IA) or (B-IB) wherein R¹⁰ and R¹¹ connect to form a C₂ or C₃alkylene.

In some embodiments, R¹ and R¹⁰ together with the -L-C(O)—N— moiety that separates them form a unsubstituted C₂-C₇heterocycloalkyl optionally fused with a phenyl ring.

In some embodiments, the group

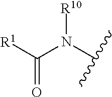

is selected from:

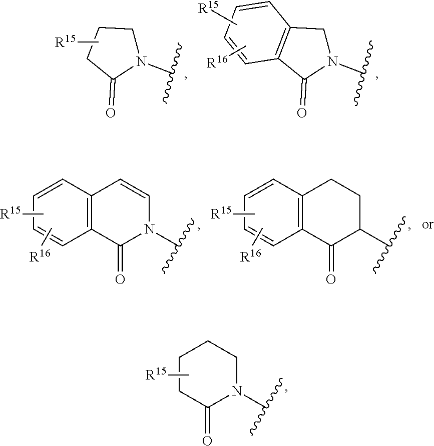

wherein R¹⁵ is H, CN, C₁-C₃ alkyl or C₃-C₅ cycloalkyl, OR¹⁹, NR¹⁷R¹⁸ (R¹⁷ and R¹⁸ are as defined herein, e.g., R¹⁷ and R¹⁸ are independently C₁-C₃ alkyl) or CN; R¹⁶ is H, F or Cl, R⁹ is C₁-C₃ alkyl or C₃-C₈ cycloalkyl.

In some embodiments, R¹⁵ is H.

In some embodiments, R¹ is bicyclo[1.1.1]pentanyl, phenyl, pyridyl, pyrimidyl or pyrazinyl, wherein the phenyl, pyridyl, pyrimidyl or pyrazinyl is substituted with a substitutent selected from isopropyl, hydroxyl substituted isopropyl, cyano substituted isopropyl, tertbutyl, hydroxyl substituted tertbutyl, cyano substituted tertbutyl, cyclopropyl, fluoro substituted cyclopropy, trifluoromethyl substituted cyclopropyl, oxetanyl, pyridyl, pyrimidyl and dimethylamino, and optionally substituted with trifluoromethyl, fluoro or chloro.

In some embodiments, the present invention provides a compound of Formula (C-I), (C-IA) or (C-IB) wherein R¹ is substituted or unsubstituted C₆-C₁₂aryl, or substituted or unsubstituted C₁-C₁₂heteroaryl. In some embodiments, the compound is a compound of Formula (C-I), (C-IA) or (C-IB) wherein R¹ is substituted or unsubstituted phenyl. In some embodiments, the compound is a compound of Formula (C-I), (C-IA) or (C-IB) wherein R¹ is substituted or unsubstituted pyridyl. In some embodiments, the compound is a compound of Formula (C-I), (C-IA) or (C-IB) wherein R¹ is substituted or unsubstituted pyrimidinyl. In some embodiments, the compound is a compound of Formula (C-I), (C-IA) or (C-IB) wherein R¹ is substituted or unsubstituted indolyl. In another embodiment is a compound of Formula (C-I), (C-IA) or (C-IB) wherein R¹ is substituted or unsubstituted benzimidazolyl. In some embodiments, the compound is a compound of Formula (C-I), (C-IA) or (C-IB) wherein R¹ is substituted or unsubstituted benzofuranyl. In some embodiments, the compound is a compound of Formula (C-I), (C-IA) or (C-IB) wherein R¹ is substituted isoindolinyl. In some embodiments, the compound is a compound of Formula (C-I), (C-IA) or (C-IB) wherein R¹ is unsubstituted isoindolinyl. In some embodiments, the compound is a compound of Formula (C-I), (C-IA) or (C-IB) wherein R¹ is —NR⁷R¹⁰. In some embodiments, the compound is a compound of Formula (C-I), (C-IA) or (C-IB) wherein R¹ is —N(CH₃)₂.

In some embodiments, R¹⁰ is hydrogen. In some embodiments, R¹⁰ is C₁-C₄alkyl, such as methyl. In some embodiments, m is 1 and R¹⁰ and R⁷ are independently C₁-C₄alkyl. In some embodiments, R⁷ is hydrogen, C₁-C₄alkyl, or C₂-C₄alkenyl. In another embodiment is a compound of Formula (C-I), (C-IA) or (C-IB) wherein R¹ is C₁-C₄alkyl substituted with —NR⁷R¹⁰, such as NH₂. In another embodiment is a compound of Formula (C-I), (C-IA) or (C-IB) wherein R¹ is C₁-C₄alkyl substituted with —NHC(O)R⁸, such as —NHC(O)CH═CH₂. In some embodiments, R⁸ is C₁-C₄alkyl. In some embodiments, R⁸ is C₂-C₄alkenyl.

In some embodiments, R⁷ is selected from

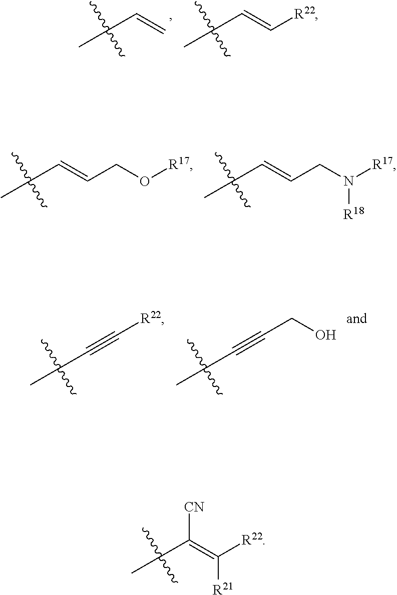

In some embodiments, R⁷ is selected from

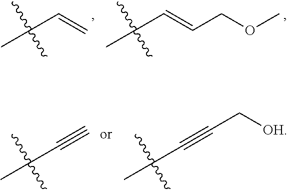

In some embodiments, the present invention provides a compound of Formula (A-I), (A-I), (A-IA)-(A-IH) or (A-VII) wherein L is a single bond. In some embodiments, the compound is a compound of Formula (A-I), (A-II), (A-IA)-(A-IH) or (A-VII) wherein L is NR¹¹.

In some embodiments, the present invention provides a compound of Formula (A-I), (A-II), (A-IA)-(A-IH) or (A-VII) wherein R¹ is substituted or unsubstituted C₆-C₁₂aryl, or substituted or unsubstituted C₁-C₁₂heteroaryl. In some embodiments, the compound is a compound of Formula (A-I), (A-II), (A-IA)-(A-IH) or (A-VII) wherein R¹ is substituted or unsubstituted phenyl. In some embodiments, the compound is a compound of Formula (A-I), (A-II), (A-IA)-(A-IH) or (A-VII) wherein R¹ is substituted or unsubstituted pyridyl. In some embodiments, the compound is a compound of Formula (A-I), (A-II), (A-IA)-(A-IH) or (A-VII) wherein R¹ is substituted or unsubstituted pyrimidinyl. In some embodiments, the compound is a compound of Formula (A-I), (A-II), (A-IA)-(A-IH) or (A-VII) wherein R¹ is substituted or unsubstituted indolyl. In some embodiments, the compound is a compound of Formula (A-I), (A-II), (A-IA)-(A-IH) or (A-VII) wherein R¹ is substituted or unsubstituted benzimidazolyl. In some embodiments, the compound is a compound of Formula (A-I), (A-II), (A-IA)-(A-IH) or (A-VII) wherein R¹ is substituted or unsubstituted benzofuranyl. In some embodiments, the compound is a compound of Formula (A-I), (A-II), (A-IA)-(A-IH) or (A-VII) wherein R¹ is substituted isoindolinyl. In some embodiments, the compound is a compound of Formula (A-I), (A-II), (A-IA)-(A-IH) or (A-VII) wherein R¹ is unsubstituted isoindolinyl.

In some embodiments, R¹ and R¹⁰ together with the -L-C(O)—N— moiety that separate them form a unsubstituted C₂-C₇heterocycloalkyl optionally fused with a phenyl ring.

In some embodiments, the group

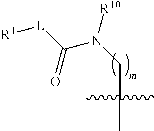

is selected from:

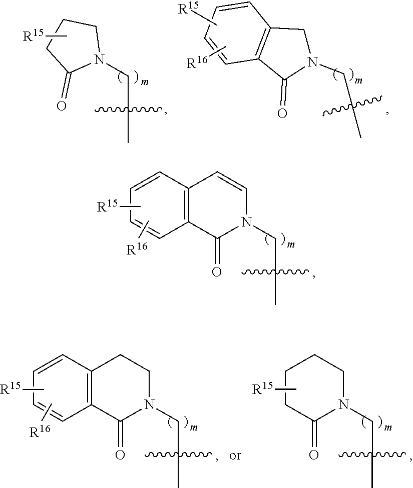

wherein R¹⁵ is H, CN, C₁-C₃ alkyl or C₃-C₈ cycloalkyl, OR¹⁹, or NR¹⁷R¹⁸ (R¹⁷ and R¹⁸ are as defined herein, e.g., R¹⁷ and R¹⁸ are independently C₁-C₃ alkyl); R¹⁶ is H, F or Cl, and R¹⁹ is C₁-C₃ alkyl or C₃-C₈ cycloalkyl.
In some embodiments, R¹ is:

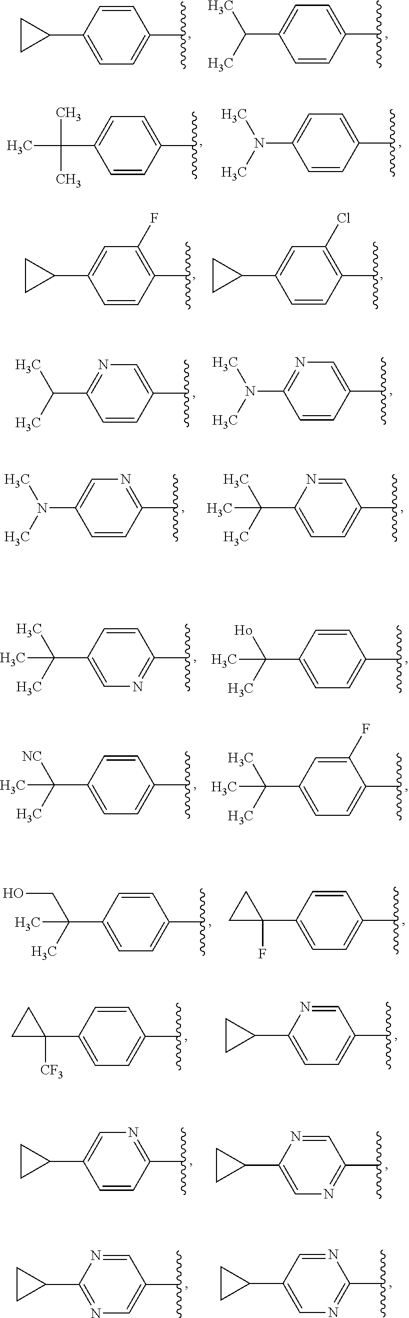

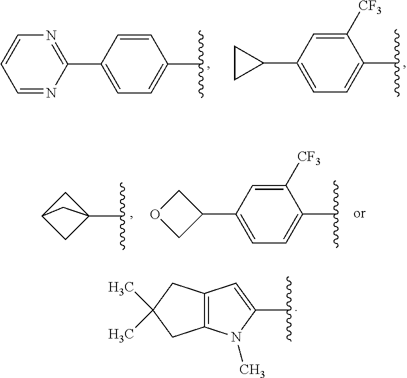

In some embodiments, the group R¹ is

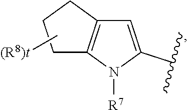

wherein each R⁷ is independently C₁-C₄alkyl, such as methyl, or halo, such as F, Cl, or Br; each R⁸ is independently H or C₁-C₃alkyl, such as methyl; t is 0, 1 or 2.

In some embodiments, the compound is a compound of Formula (A-I), (A-II), (A-IA)-(A-IH) or (A-VII) wherein R¹ is substituted or unsubstituted C₂-C₄alkenyl, substituted or unsubstituted C₂-C₄alkynyl, substituted or unsubstituted cyclohexyl, substituted C₂-C₇heterocycloalkyl, substituted C₆-C₁₂aryl, or substituted or unsubstituted C₁-C₁₂heteroaryl; or R¹ is substituted or unsubstituted isoindolinyl or CN; in some embodiments, R¹ is 2-substituted phenyl or 3-substituted phenyl.

In some embodiments, the compound is a compound of Formula (A-I). (A-II), (A-IA)-(A-IH) or (A-VII) wherein R¹ is other than

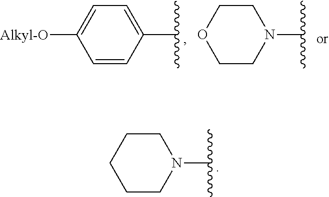

In other embodiments, R¹ is other than a phenyl substituted with one substitutent at the 4-position. In other embodiments, R¹ is substituted phenyl and the substitution is at the 2-, or 3-position.

In some embodiments, the present invention provides compounds of Formula (A-IIa) having the structure:

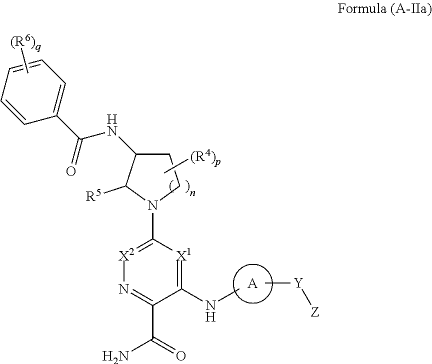

wherein:
A, X¹, X², Y, Z, R⁴, R⁵, n and p are as defined herein;
each R⁶ is independently halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, and/or pharmaceutically acceptable prodrug thereof.

In some embodiments, the present invention provides compounds of Formula (A-IIb) having the structure:

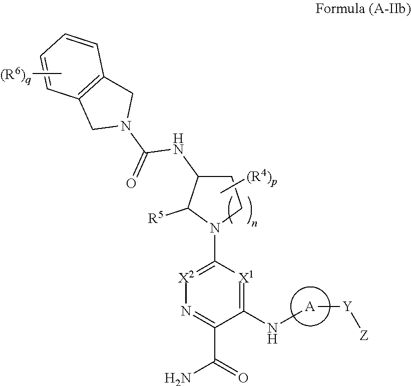

wherein:
A, X¹, X², Y, Z, R⁴, R⁵, n and p are as defined herein;
each R⁶ is independently halogen, —CN, —OH, —NH₂, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; and
q is 0, 1, 2 or 3;
or a pharmaceutically acceptable solvate, pharmaceutically acceptable salt, and/or pharmaceutically acceptable prodrug thereof.

In some embodiments, the present invention provides compounds of Formula (A-IIIa) having the structure:

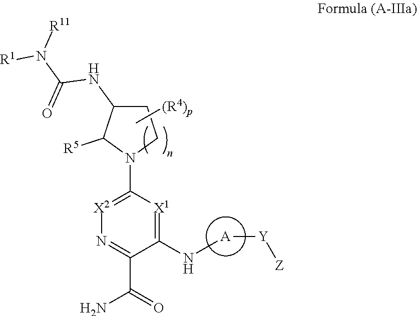

wherein:
A, X¹, X², Y, Z, R¹, R⁵, R¹¹, n and p are as defined herein;
or a pharmaceutically acceptable solvate, pharmaceutically acceptable salt, and/or pharmaceutically acceptable prodrug thereof.

In some embodiments, the present invention provides compounds of Formula (A-IIIb) having the structure:

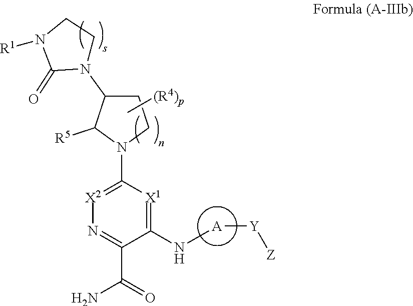

wherein:
A, X¹, X², Y, Z, R¹, R⁴, R⁵, n and p are as defined herein, and s is 1, 2 or 3;
or a pharmaceutically acceptable solvate, pharmaceutically acceptable salt, and/or pharmaceutically acceptable prodrug thereof.

In some embodiments, the present invention provides compounds of Formula (A-VI) having the structure:

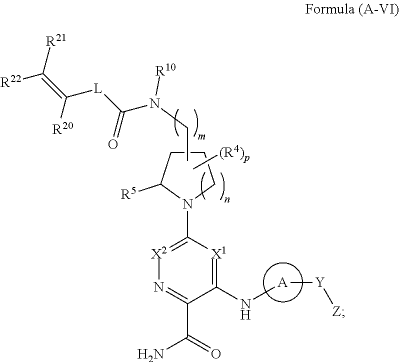

wherein:
wherein A, L, X¹, X², Y, Z, R⁴, R⁵, R¹⁰, R²⁰, R²¹, R²², m, n and p are as defined herein;
or a pharmaceutically acceptable solvate, pharmaceutically acceptable salt, and/or pharmaceutically acceptable prodrug thereof.

In some embodiments, the present invention provides compounds of Formula (B-IIa):

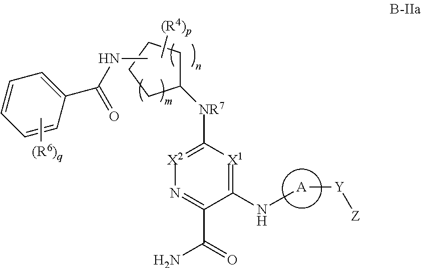

wherein:
A, X¹, X², Y, Z, R⁴, R⁷, m, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In some embodiments, the present invention provides compounds of Formula (B-IIb) having the structure:

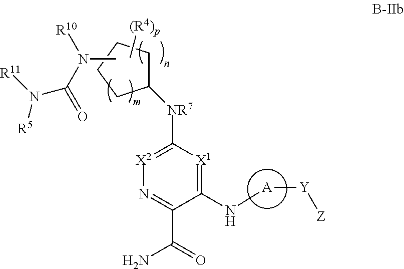

wherein:
A, X¹, X², Y, Z, R⁴, R⁵, R⁷, R¹⁰, R¹¹, m, n and p are as defined herein;
or a pharmaceutically acceptable solvate, pharmaceutically acceptable salt, or pharmaceutically acceptable prodrug thereof.

In some embodiments, the present invention provides compounds of Formula (B-IIc) having the structure:

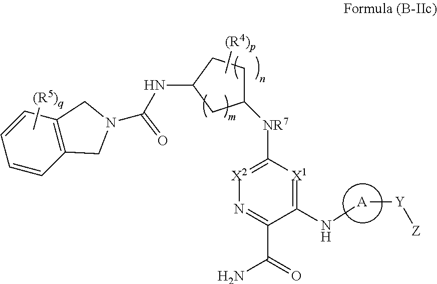

wherein:
A, X¹, X², Y, Z, R⁴, R⁷, m, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In some embodiments, the present invention provides compounds of Formula (B-IId) having the structure:

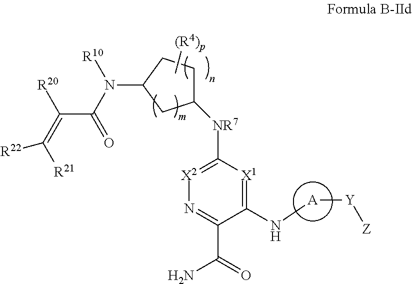

wherein:
A, X¹, X², Y, Z, R⁴, R⁷, R¹⁰, m, n and p are as defined herein;
R²⁰, R²¹ and R²² are each independently H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, or pharmaceutically acceptable prodrug thereof.

In some embodiments, in Formulas (A-IIa), (A-IIb), (A-IIIa), (A-IIIb), (A-VI), (B-IIa), (B-IIb), (B-IIc), or (B-IId), R²⁰ and R²¹ are H, R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₂aryl, or substituted or unsubstituted C₁-C₁₂heteroaryl. In some embodiments, all of R²⁰, R²¹ and R²² are H. In some embodiments, R²⁰ and R²¹ together form a bond and R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R²⁰ is CN. In some embodiments, R²⁰ is halo, such as F.

In some embodiments, in Formulas (A-IIa), (A-IIb), (A-IIIa), (A-IIIb), (A-VI), (B-IIa), (B-IIb), (B-IIc), or (B-IId), R²⁰, R²¹ and R²² are independently H, F, Cl, C₁-C₄ alkyl or cycloalkyl, CF₃, or CN. In some embodiments, one of R²⁰ and R²¹ is H, the other one of R²⁰ and R²¹ is F, Cl, C₁-C₄ alkyl, C₃-C₈ cycloalkyl, CF₃, or CN, and R²² is H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl.

In another aspect, provided herein is a compound of the formula:

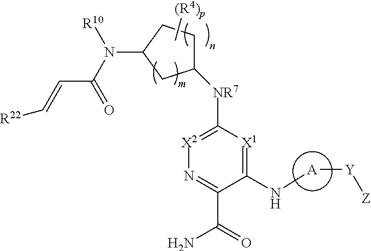

or a pharmaceutically acceptable solvate, pharmaceutically acceptable salt, or pharmaceutically acceptable prodrug thereof; wherein A, X¹, X², Y, Z, R⁴, R⁷, R¹⁰, R²², m, n and p are as defined herein.

In another aspect, provided herein is a compound of the formula:

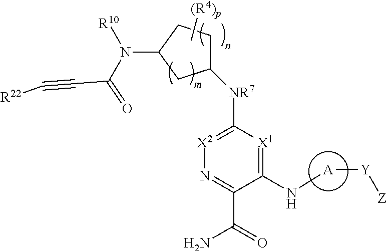

or a pharmaceutically acceptable solvate, pharmaceutically acceptable salt, or pharmaceutically acceptable prodrug thereof; wherein A, X¹, X², Y, Z, R⁴, R⁷, R¹⁰, R²², m, n and p are as defined herein.

In another aspect, provided herein is a compound of Formula (B-VIII) having the structure:

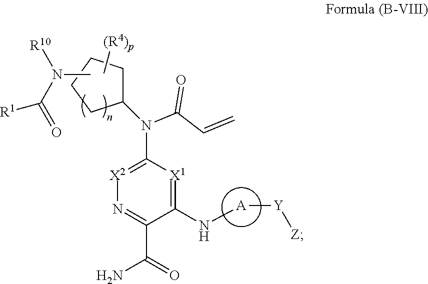

or a pharmaceutically acceptable solvate, pharmaceutically acceptable salt, or pharmaceutically acceptable prodrug thereof, the variables are as defined herein.

In some embodiments the present invention provides a compound of Formula (A-I), (A-II), (A-VI), (A-IA)-(A-IH), (A-IIa), (A-IIb), (A-IIIa), (A-IIIb) or (A-VII) wherein ring A is substituted or unsubstituted C₆-C₁₂aryl. In some embodiments, the compound is a compound of Formula (A-I), (A-II), (A-VI), (A-IA)-(A-IH), (A-IIa), (A-IIb), (A-IIIa), (A-IIIb) or (A-VII) wherein ring A is phenyl. In some embodiments, the compound is a compound of Formula (A-I), (A-II), (A-VI), (A-IA)-(A-IH), (A-IIa), (A-IIb), (A-IIIa), (A-IIIb) or (A-VII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (A-I), (A-II), (A-VI), (A-IA)-(A-IH), (A-IIa), (A-IIb), (A-IIIa), (A-IIIb) or (A-VII) wherein Y is a single bond, —CH₂O—, —OCH₂—, —O—, —N(R³)—, —N(R³)C(O)—, —C(O)N(R³)—, or substituted or unsubstituted C₁-C₄alkylene. In some embodiments, the compound is a compound of Formula (A-I), (A-II), (A-VI), (A-IA)-(A-IH), (A-IIa), (A-IIb), (A-IIIa), (A-IIIb) or (A-VII) wherein Y is a single bond, —C(O)—, or —C(O)N(R³)—. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is substituted or unsubstituted C₁-C₃alkyl. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is Me, Et, or i-Pr. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments the present invention provides a compound of Formula (A-I), (A-II), (A-VI), (A-IA)-(A-IH), (A-IIa), (A-IIb), (A-IIIa), (A-IIIb) or (A-VII) wherein ring A is substituted or unsubstituted C₁-C₁₂heteroaryl. In some embodiments, the compound is a compound of Formula (A-I), (A-II), (A-VI), (A-IA)-(A-IH), (A-IIa), (A-IIb), (A-IIIa), (A-IIIb) or (A-VII) wherein ring A is pyridyl. In some embodiments, the compound is a compound of Formula (A-I), (A-II), (A-VI), (A-IA)-(A-IH), (A-IIa), (A-IIb), (A-IIIa), (A-IIIb) or (A-VII) wherein A is isothiazolyl. In some embodiments, the compound is a compound of Formula (A-I), (A-II), (A-VI), (A-IA)-(A-IH), (A-IIa), (A-IIb), (A-IIIa), (A-IIIb) or (A-VII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (A-I), (A-II), (A-VI), (A-IA)-(A-IH), (A-IIa), (A-IIb), (A-IIIa), (A-IIIb) or (A-VII) wherein Y is a single bond, —C(O)—, or —C(O)N(R³)—. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is substituted or unsubstituted C₁-C₃alkyl. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is Me, Et, or i-Pr. In some embodiments, the compound is a compound of Formula (A-I), (A-II), (A-VI), (A-IA)-(A-IH), (A-IIa), (A-IIb), (A-IIIa), (A-IIIb) or (A-VII) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments, the compound is a compound of Formula (A-I), (A-II), (A-VI), (A-IA)-(A-IH), (A-IIa), (A-IIb), (A-IIIa), (A-IIIb) or (A-VII) wherein A is isothiazolyl; Y is a single bond; and Z is Me.

In some embodiments the present invention provides a compound of Formula (B-I), (B-II), (B-IA), (B-IB), (B-IIa)-(B-IId) or (B-VIII) wherein ring A is substituted or unsubstituted C₆-C₁₂aryl. In some embodiments, the compound is a compound of Formula (B-I), (B-II), (B-IA), (B-IB), (B-IIa)-(B-IId) or (B-VIII) wherein ring A is phenyl. In another embodiment is a compound of Formula (B-I), (B-II), (B-IA), (B-IB), (B-IIa)-(B-IId) or (B-VIII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (B-I), (B-II), (B-IA), (B-IB), (B-IIa)-(B-IId) or (B-VIII) wherein Y is a single bond, —C(O)—, or —C(O)N(R³)—. In some embodiments, the compound is a compound of Formula (B-I), (B-II), (B-IA), (B-IB), (B-IIa)-(B-IId) or (B-VIII) wherein Z is substituted or unsubstituted C₁-C₃alkyl. In some embodiments, the compound is a compound of Formula (B-I), (B-II), (B-IA), (B-IB), (B-IIa)-(B-IId) or (B-VIII) wherein Z is Me, Et, or i-Pr. In some embodiments, the compound is a compound of Formula (B-I), (B-II), (B-IA), (B-IB), (B-IIa)-(B-IId) or (B-VIII) wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments, m is 1. In some embodiments, m is 2. In some embodiments, m is 3.

In some embodiments the present invention provides a compound of Formula (B-I), (B-II), (B-IA), (B-IB), (B-IIa)-(B-IId) or (B-VIII) wherein ring A is substituted or unsubstituted C₁-C₁₂heteroaryl. In some embodiments, the compound is a compound of Formula (B-I), (B-II), (B-IA), (B-IB), (B-IIa)-(B-IId) or (B-VIII) wherein ring A is pyridyl. In another embodiment is a compound of Formula (B-I), (B-II), (B-IA), (B-IB), (B-IIa)-(B-IId) or (B-VIII) wherein A is isothiazolyl. In some embodiments, the compound is a compound of Formula (B-I), (B-II), (B-IA), (B-IB), (B-IIa)-(B-IId) or (B-VIII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments, the compound is a compound of Formula (B-I), (B-II), (B-IA), (B-IB), (B-IIa)-(B-IId) or (B-VIII) wherein Y is a single bond, —C(O)—, or —C(O)N(R³)—. In some embodiments, the compound is a compound of Formula (B-I), (B-II), (B-IA), (B-IB), (B-IIa)-(B-IId) or (B-VIII) wherein Z is substituted or unsubstituted C₁-C₃alkyl. In some embodiments, the compound is a compound of Formula (B-I), (B-II), (B-IA), (B-IB), (B-IIa)-(B-IId) or (B-VIII) wherein Z is Me, Et, or i-Pr. In some embodiments, the compound is a compound of Formula (B-I), (B-II), (B-IA), (B-IB), (B-IIa)-(B-IId) or (B-VIII) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments, the compound is a compound of Formula (B-I), (B-II), (B-IA), (B-IB), (B-IIa)-(B-IId) or (B-VIII) wherein A is isothiazolyl; Y is a single bond; and Z is Me.

In some embodiments, -A-Y—Z is substituted or unsubstituted 5-membered heteroaryl. In some embodiments, the 5-membered heteroaryl is a 5-membered heteroaryl having one sulfur atom and optionally a nitrogen atom, e.g., isothiazole or thiazole. In some embodiments, -A-Y—Z is thiazole or isothiazole substituted with C₁-C₃alkyl, such as isopropyl, or phenyl.

In some embodiments, R¹ is substituted or unsubstituted C₁-C₄alkenyl, and -A-Y—Z is unsubstituted 5-membered heteroaryl. In some embodiments, R¹ is substituted or unsubstituted C₁-C₄alkenyl, and -A-Y—Z is 5-membered heteroaryl substituted with C₁-C₃alkyl or phenyl. In some embodiments, the 5-membered heteroaryl is a 5-membered heteroaryl having one sulfur atom and optionally a nitrogen atom, e.g., isothiazole or thiazole. In some embodiments, -A-Y—Z is isothiazole substituted with C₁-C₃alkyl, such as isopropyl, or phenyl. In some embodiments, R¹ is unsubstituted C₁-C₄alkenyl, and -A-Y—Z is phenyl substituted with one or two substituents independently selected from C₁-C₃alkyl, 5- or 6-membered heteroaryl, or C(O)NHR⁹, wherein R⁹ is phenyl substituted with one or two C₁-C₃alkyl. In some embodiments, -A-Y—Z is phenyl substituted with an isopropyl and optionally a methyl. In some embodiments, -A-Y—Z is phenyl substituted with pyridyl. In some embodiments, -A-Y—Z is phenyl substituted with C(O)NHR⁹. In some embodiments, R⁹ is phenyl substituted an isopropyl and optionally a methyl.

In some embodiments, -A-Y—Z is

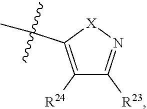

wherein X is O or S, R²³ and R²⁴ are independently H, C₁-C₄alkyl, halo, CN, CONR^{2S}R²⁶, CH₂NR²⁵R²⁶, aryl or heteroaryl, wherein R²⁵ and R²⁶ are independently H or C₁-C₄alkyl. In some embodiments, X is O. In some embodiments, X is S.

In some embodiments, -A-Y—Z

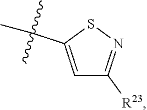

wherein R²³ is CN, CONR²⁵R²⁶, CH₂NR²⁵R²⁶, aryl or heteroaryl, wherein R²⁵ and R²⁶ are independently H or C₁-C₄alkyl.

In some embodiments, -A-Y—Z is

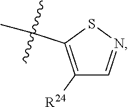

wherein R²⁴ is C₁-C₄alkyl, such as methyl, or halo, such as F, Cl, or Br.

In some embodiments, -A-Y—Z is

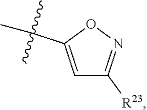

wherein R²³ is CN, CONR²⁵R²⁶, CH₂NR²⁵R²⁶, aryl or heteroaryl, wherein R²⁵ and R²⁶ are independently H or C₁-C₄alkyl.

In some embodiments, -A-Y—Z is

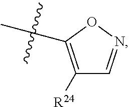

wherein R²⁴ is C₁-C₄alkyl, such as methyl, or halo, such as F, Cl, or Br.

In some embodiments, -A-Y—Z is

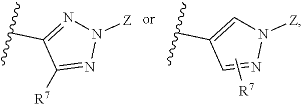

wherein R⁷ is C₁-C₄alkyl, such as methyl, or halo, such as F, Cl, or Br, and Z is H or C₁-C₃alkyl optionally substituted with halo, alkoxy or N(R³⁰)₂ (wherein R³⁰ is each independently H or C₁-C₃alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl.

In some embodiments, -A-Y—Z is

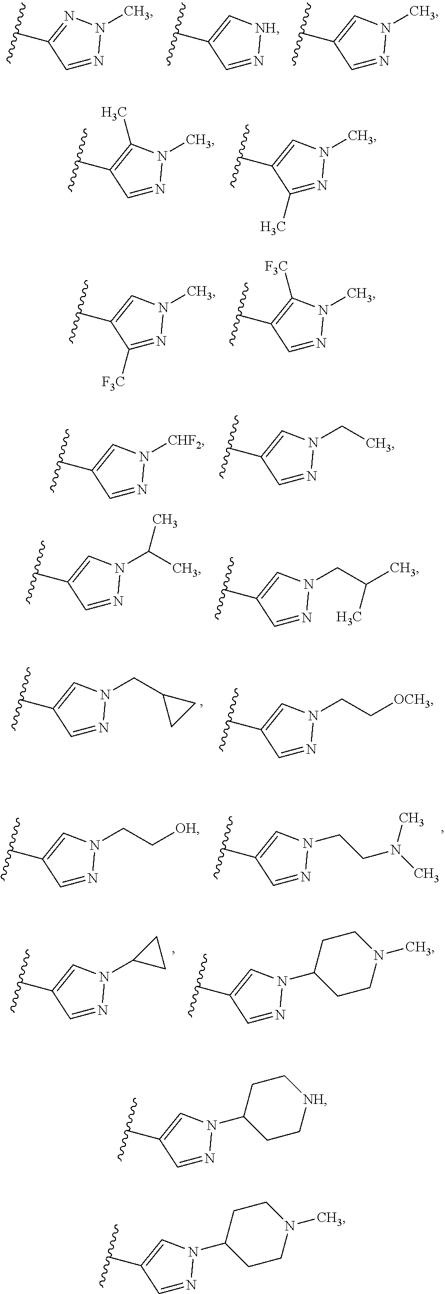

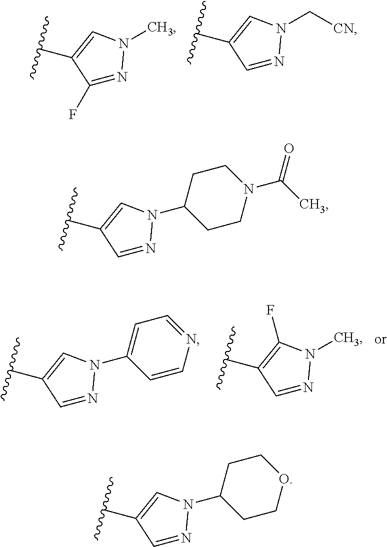

In some embodiments, the present invention provides a compound of Formula (A-I), (A-II), (A-VI), (A-IA)-(A-IH), (A-IIa), (A-IIb), (A-IIIa), (A-IIIb) or (A-VII) wherein A is substituted or unsubstituted C₁-C₁₂heteroaryl. In some embodiments, the present invention provides a compound of Formula (A-I), (A-II), (A-VI), (A-IA)-(A-IH), (A-IIa), (A-IIb), (A-IIIa), (A-IIIb) or (A-VII) wherein Y is a single bond; in some embodiments, the present invention provides a compound of Formula (A-I), (A-II), (A-VI), (A-IA)-(A-IH), (A-IIa), (A-IIb), (A-IIIa), (A-IIIb) or (A-VII) wherein Z is H, substituted or unsubstituted C₁-C₃alkyl, substituted or unsubstituted C₃-C₆cycloalkyl, substituted or unsubstituted C₆-C_{12z}aryl, or substituted or unsubstituted C₁-C₁₂heteroaryl.

In some embodiments the present invention provides a compound of Formula (A-I), (A-I), (A-VI), (A-IA)-(A-IH), (A-IIa), (A-IIb), (A-IIa), (A-IIIb) or (A-VII) wherein A-Y—Z is other than

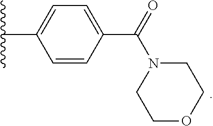 [SMILES: *c1ccc(C(=O)N2CCOCC2)cc1]

In some embodiments, the group -A-Y—Z is:

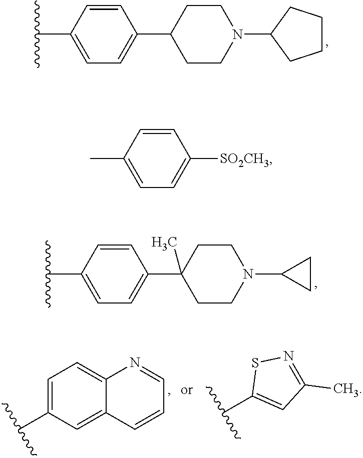 [SMILES: *c1ccc(C2CCN(C3CCCC3)CC2)cc1 ; Cc1ccc(S(C)(=O)=O)cc1 ; *c1ccc(C2(C)CCN(C3CC3)CC2)cc1 ; *c1ccc2ncccc2c1 ; *c1cc(C)ns1]

In some embodiments, the group -A-Y—Z is

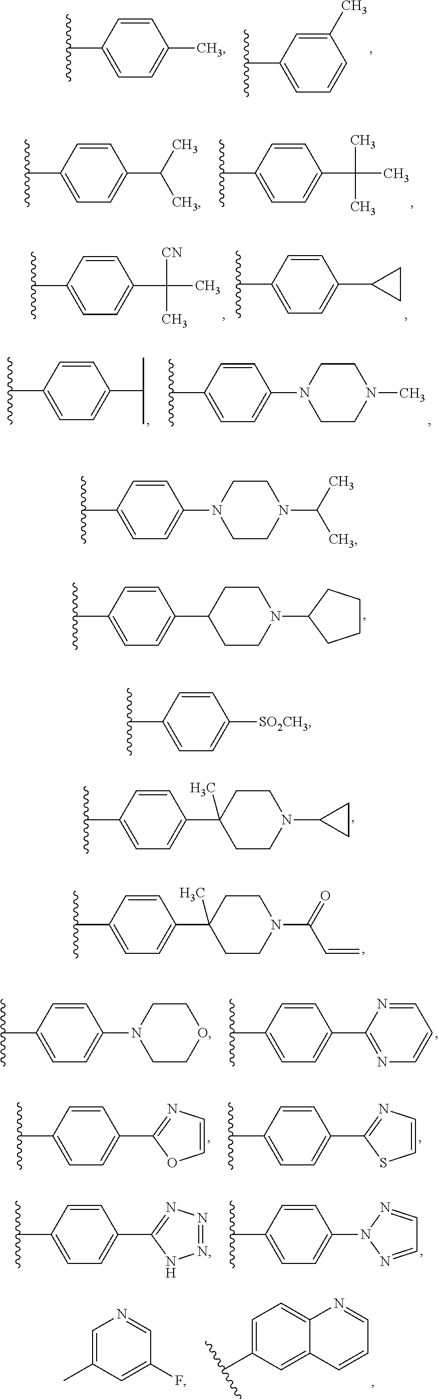

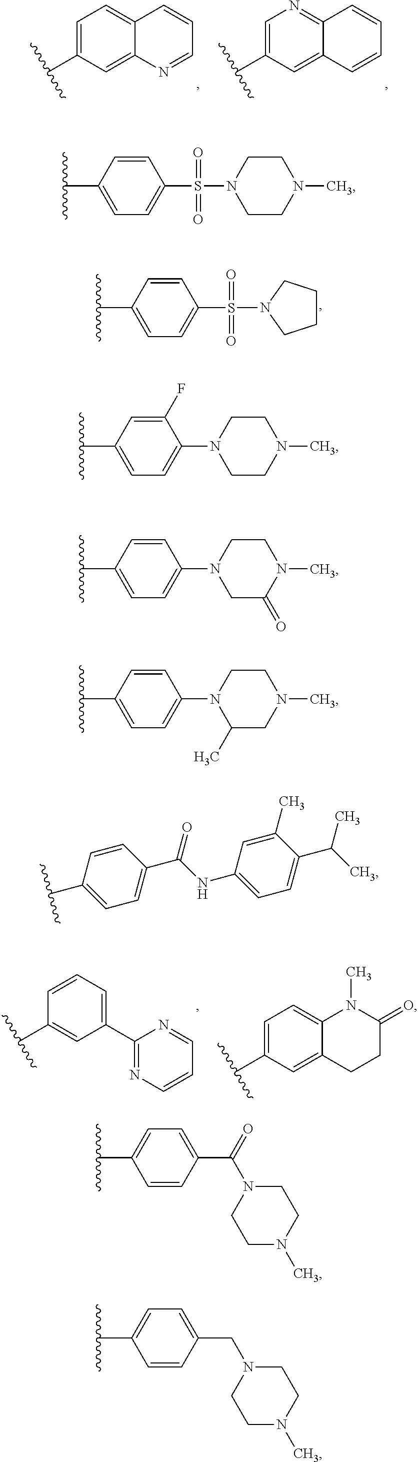

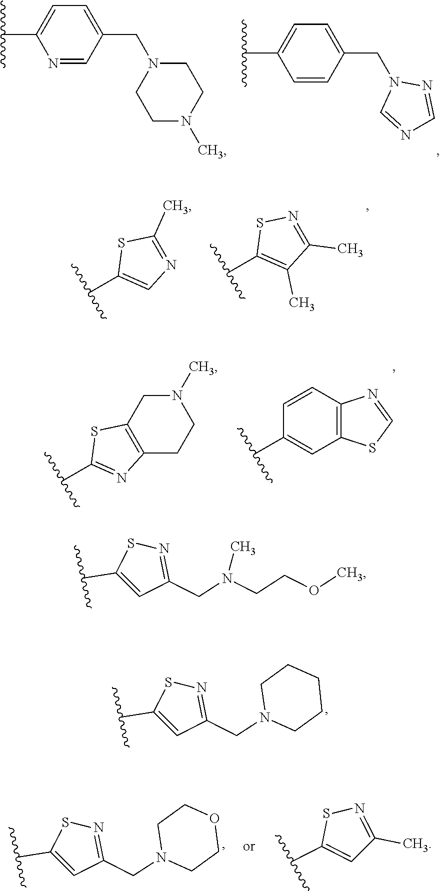

In some embodiments, the group -A-Y—Z is:

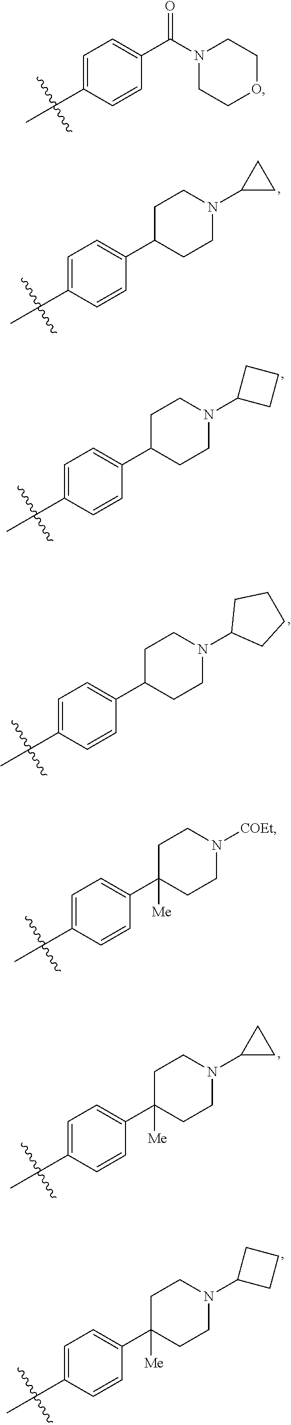

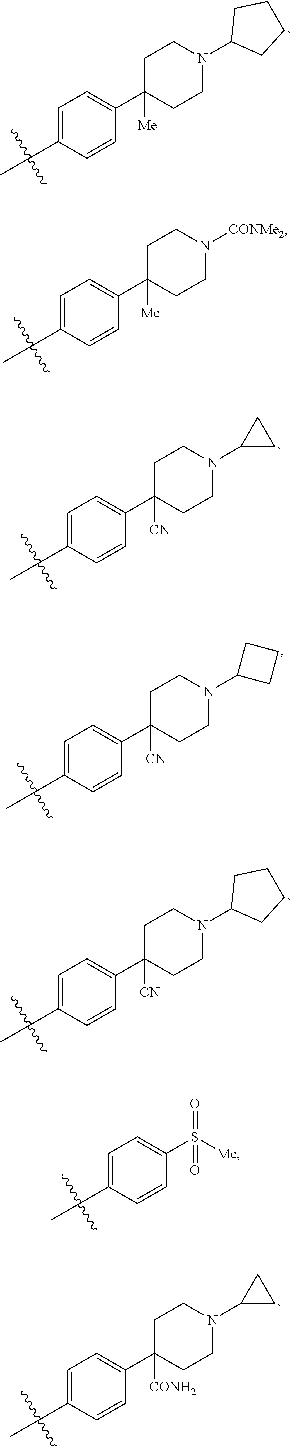

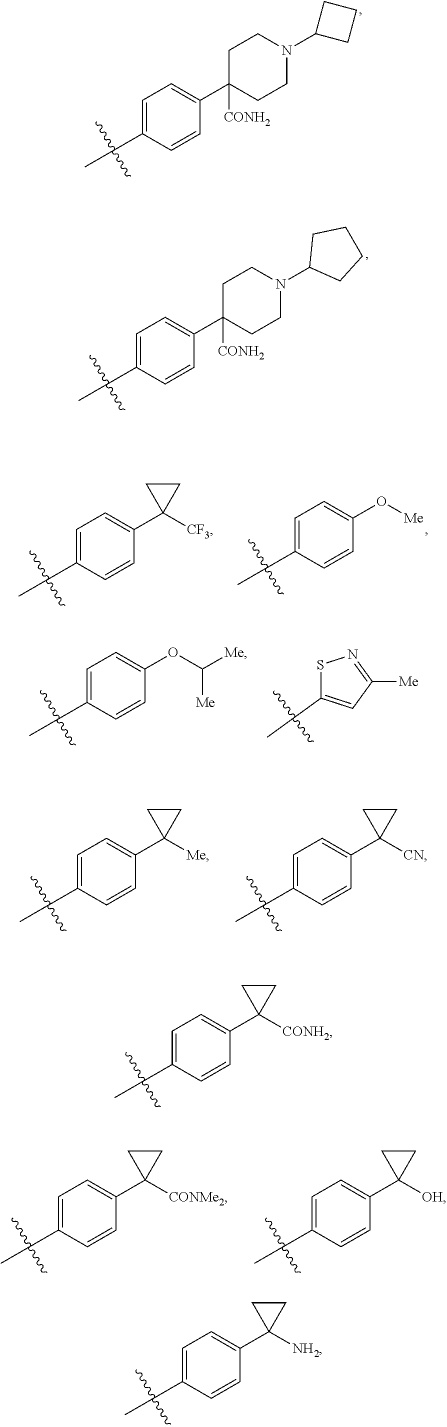

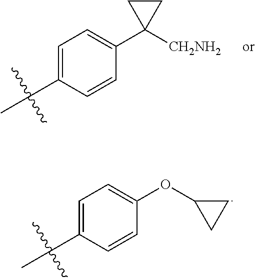

In some embodiments, the group -A-Y—Z is:

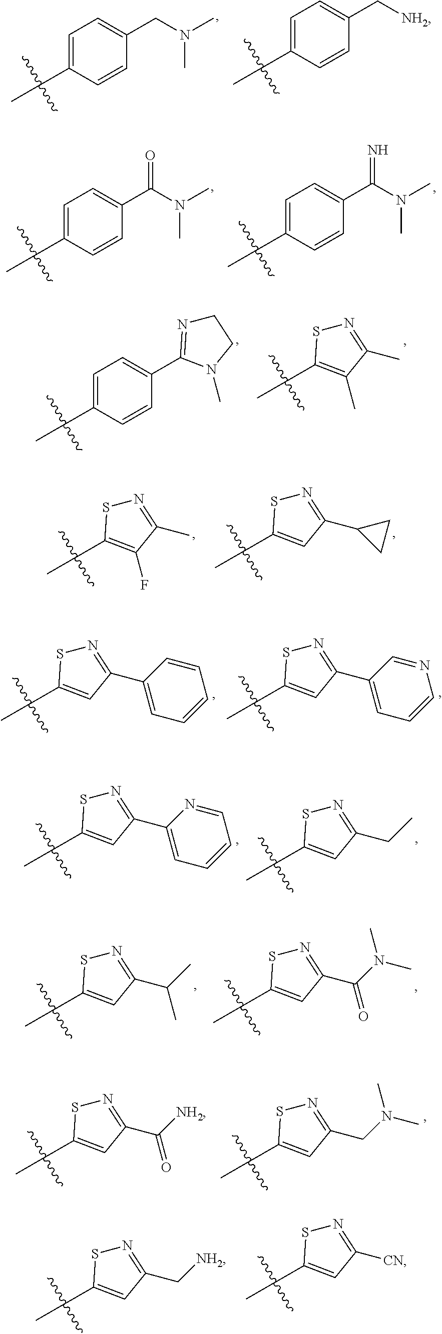

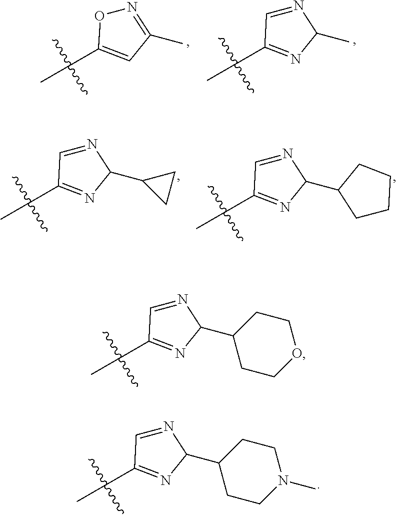

In some embodiments, -A-Y—Z is other than a substituted phenyl wherein at least one of the substituent is a substituted or unsubstituted 6-membered heterocycloalkyl.

In another aspect, provided herein is a compound of Formula (A-IVa), (A-IVb), (A-IVc), (A-IVd), (A-IVe), (A-IVf), (A-IVg), (A-IVh), (A-Va), (A-Vb), (A-Vc), (A-Vd), (A-Ve), (A-Vf), (A-Vg) or (A-Vh), having the structure:

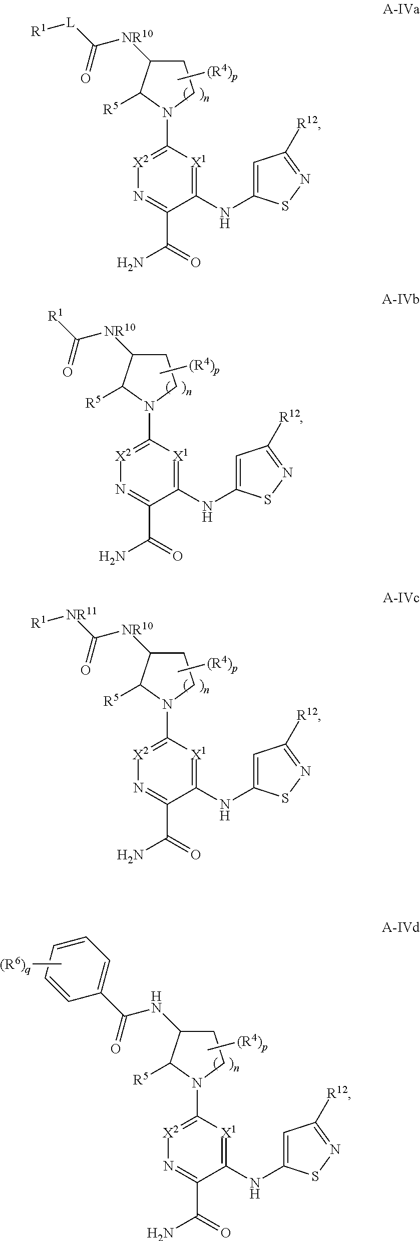

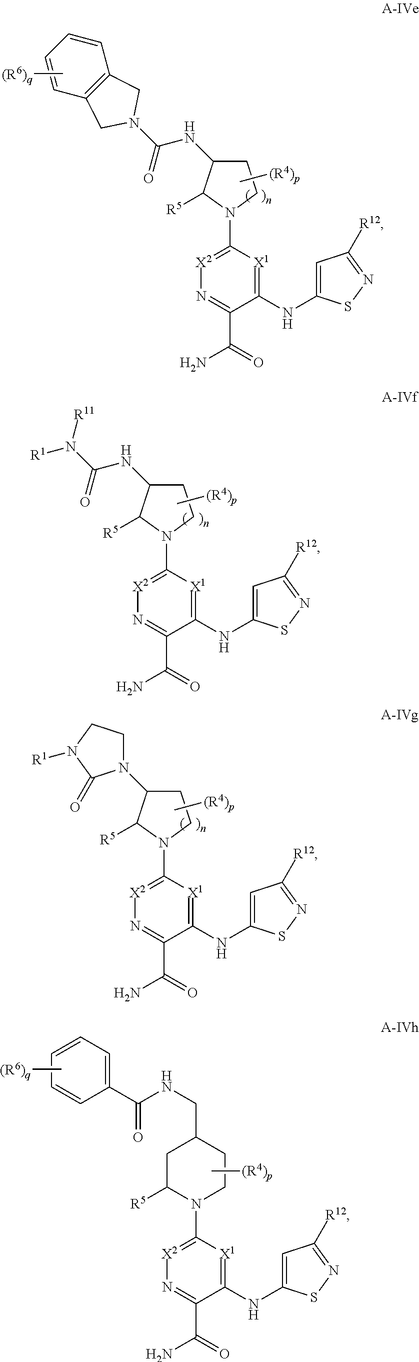

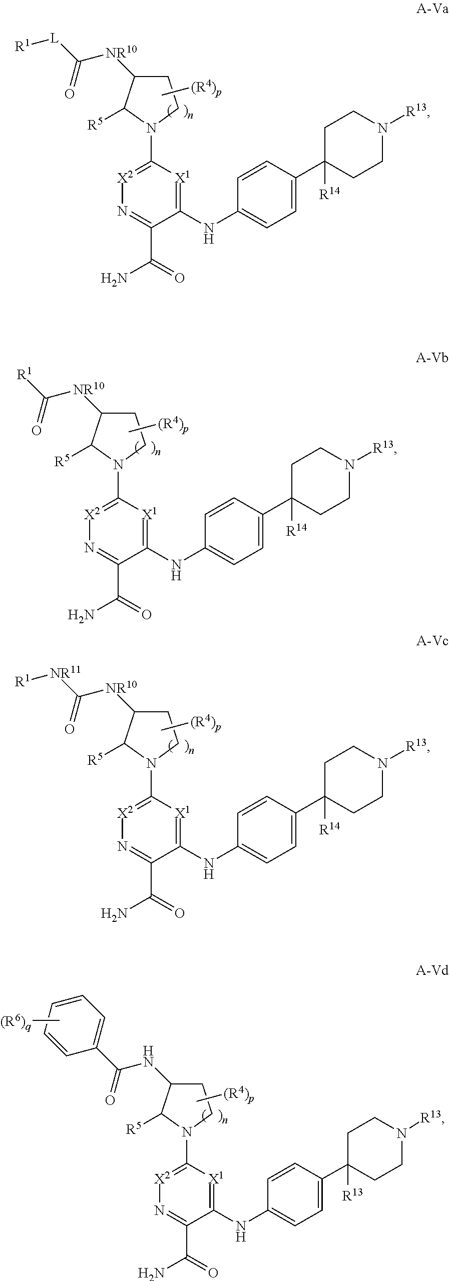

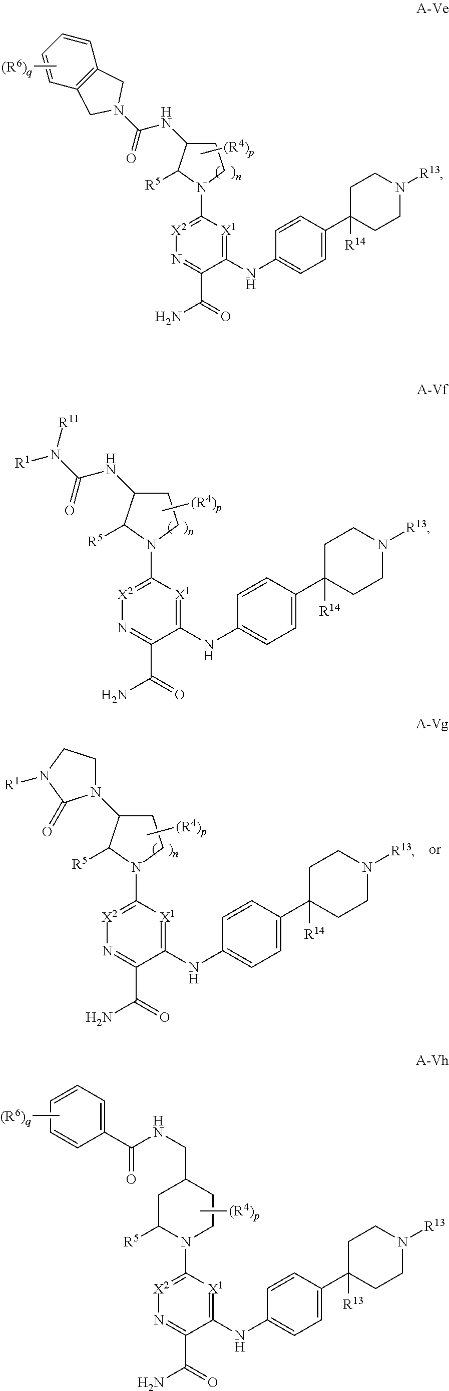

wherein:
L, X¹, X², R¹, R⁴, R⁵, R⁶, R¹⁰, R¹¹, n, p and q are as defined herein;
R¹² is substituted or unsubstituted C₁-C₃alkyl,
R¹³ is substituted or unsubstituted C₃-C₇cycloalkyl,
R¹⁴ is hydrogen or unsubstituted C₁-C₃alkyl,
or a pharmaceutically acceptable solvate, pharmaceutically acceptable salt, and/or pharmaceutically acceptable prodrug thereof.

In one embodiment, R¹² is H. In another embodiment, R¹³ is H. In another embodiment, R¹⁴ is H.

In some embodiments, R¹² is unsubstituted C₁-C₃alkyl, such as methyl or ethyl.

In some embodiments, R¹³ is unsubstituted C₃-C₇alkyl, such as cyclopropyl, cyclobutyl, cyclopentyl, cyclohexyl or cycloheptyl.

In some embodiments of any of Formula (A-I), (A-II), (A-VI), (A-IA)-(A-IE), (A-IIa), (A-IIb), (A-IIIa), (A-IIIb), (A-VII), (A-IVa)-(A-IVh) or (A-Va)-(A-Vh), X¹ and X² are both N. In some embodiments of any of Formula (A-I), (A-II), ((A-VI), ((A-IA)-((A-IE), ((A-IIa), ((A-IIb), ((A-IIIa), ((A-IIb), ((A-VII), ((A-IVa)-((A-IVh) or ((A-Va)-((A-Vh), X¹ and X² are independently C(R²). In some embodiments, X¹ and X² are both CH. In some embodiments of any of Formula (A-I), (A-II), ((A-VI), ((A-IA)-((A-IE), ((A-IIa), ((A-IIb), ((A-IIIa), ((A-IIIb), ((A-VII), ((A-IVa)-((A-IVh) or ((A-Va)-((A-Vh), X¹ is N and X² is C(R²). In some embodiments, X¹ is N and X² is CH. In some embodiments, each R² is independently H, substituted or unsubstituted C₁-C₄alkyl, —CN, or halogen.

In some embodiments of the aforementioned embodiments the compound is a compound of Formula (A-I), (A-II), (A-IA)-(A-IH), (A-IIa), (A-IIb), (A-IIIa), (A-IIIb), (A-VI), (A-IVa)-(A-IVh) or (A-Va)-(A-Vh) wherein R⁵ is H. In further embodiments of the aforementioned embodiments is a compound of Formula (A-I), (A-II), (A-IA)-(A-IH), (A-IIa), (A-IIb), (A-IIIa), (A-IIIb), (A-VI), (A-IVa)-(A-IVh) or (A-Va)-(A-Vh) wherein R⁵ is unsubstituted C₁-C₄alkyl. In further embodiments of the aforementioned embodiments the compound is a compound of Formula (A-I), (A-II), (A-IA)-(A-IH), (A-IIa), (A-IIb), (A-IIIa), (A-IIIb), (A-VI), (A-IVa)-(A-IVh) or (A-Va)-(A-Vh) wherein R⁵ is unsubstituted C₁-C₄alkyl, such as —CH₂OH. In further embodiments, p is 1 and R⁴ is C₁-C₄alkyl, such as methyl. In further embodiments, p is 1 and R⁵ together with one R⁴ is C₁-C₄alkylene. In further embodiments, p is 2 and the two R⁴ form a C₁-C₄alkylene. In further embodiments, p is 2 and the two R⁴ are independently halo. In further embodiments, both R⁴ are fluoro.

In some embodiments, the group is

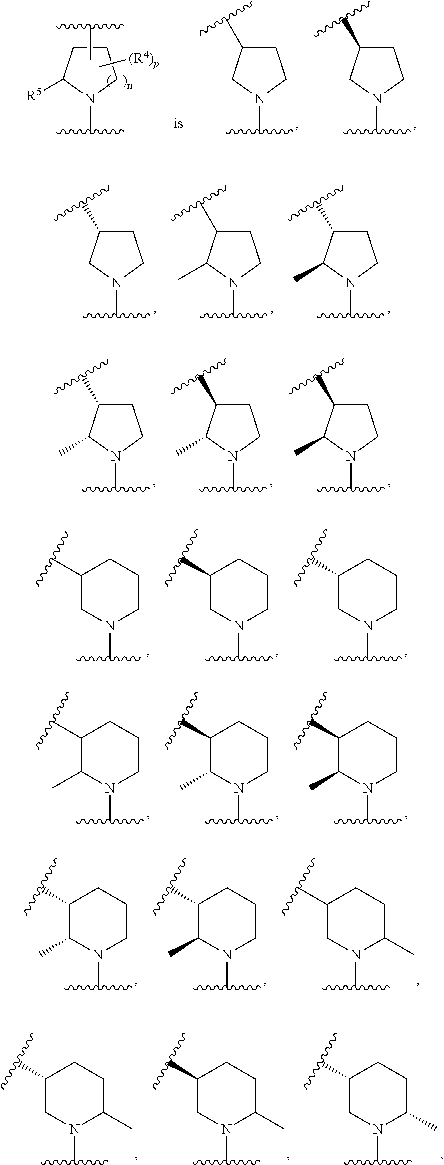

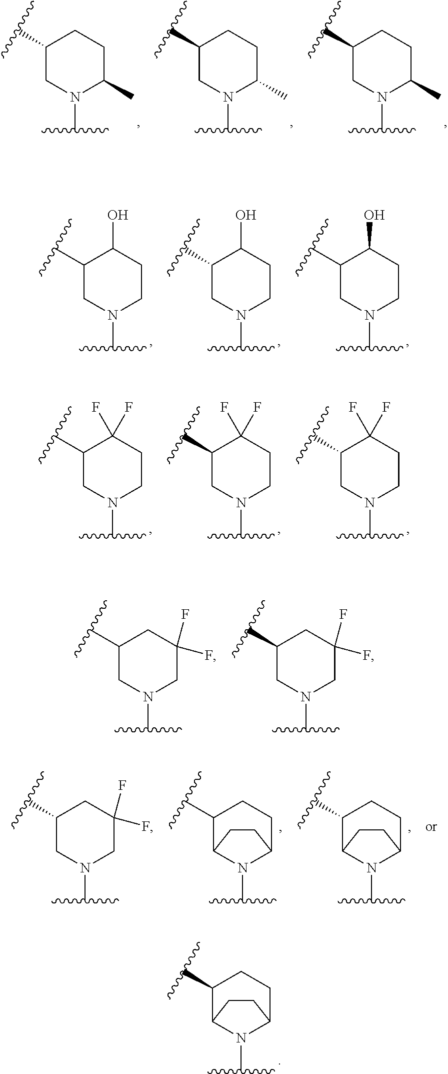

In some embodiments, the present invention is a compound of Formula (A-IIa), (A-IIb), (A-IVd), (A-IVe), (A-IVh), (A-Vd), (A-Ve) or (A-Vh) wherein q is 1. In another embodiment is a compound of Formula (A-IIa), (A-IIb), (A-IVd), (A-IVe), (A-IVh), (A-Vd), (A-Ve) or (A-Vh) wherein q is 2 or 3. In some embodiments, the compound is a compound of Formula (A-IIa), (A-IIb), (A-IVd), (A-IVe), (A-IVh), (A-Vd), (A-Ve) or (A-Vh), wherein R⁶ is independently Me, Et, i-Pr, cyclopropyl, cyclobutyl, cyclopentyl, Cl, F, amino, or dimethylamino. In some embodiments, the compound is a compound of Formula (A-IIa), (A-IIb), (A-IVd), (A-IVe), (A-IVh), (A-Vd), (A-Ve) or (A-Vh), wherein at least one R⁶ is independently F, CF₃, OCH₃, or OCF₃. In some embodiments, the compound is a compound of Formula (A-IIa), (A-IIb), (A-IVd), (A-IVe), (A-IVh), (A-Vd), (A-Ve) or (A-Vh), wherein R⁶ is independently F, CF₃, OCH₃, or OCF₃. In some embodiments, the compound is a compound of Formula (A-IIa), (A-IIb), (A-IVd), (A-IVe), (A-IVh), (A-Vd), (A-Ve) or (A-Vh) wherein at least one R⁶ is independently —N(R³)₂, or —OH. In some embodiments, at least one R⁶ is —NH₂. In some embodiments, at least one R⁶ is —N(R³)₂. In some embodiments, at least one R⁶ is —N(CH₃)₂ or —NH₂. In some embodiments, at least one R¹ is —OH.

In some embodiments, the compound is:

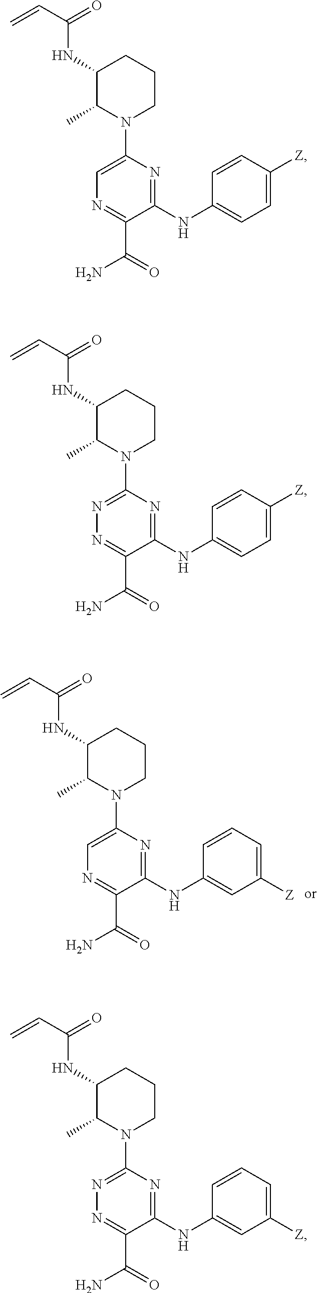

wherein Z is a 5- or 6-membered heteroaryl optionally substituted with methyl, ethyl or hydoxyl, such as

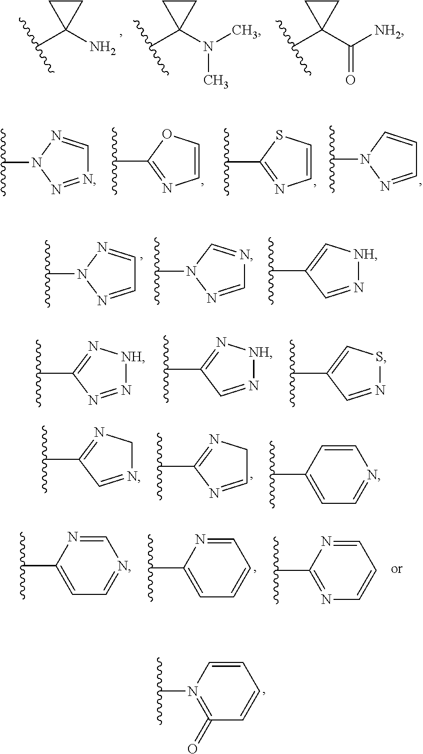

or a pharmaceutically acceptable solvate, pharmaceutically acceptable salt, and/or pharmaceutically acceptable prodrug thereof.

In some embodiments, the compound is selected from:

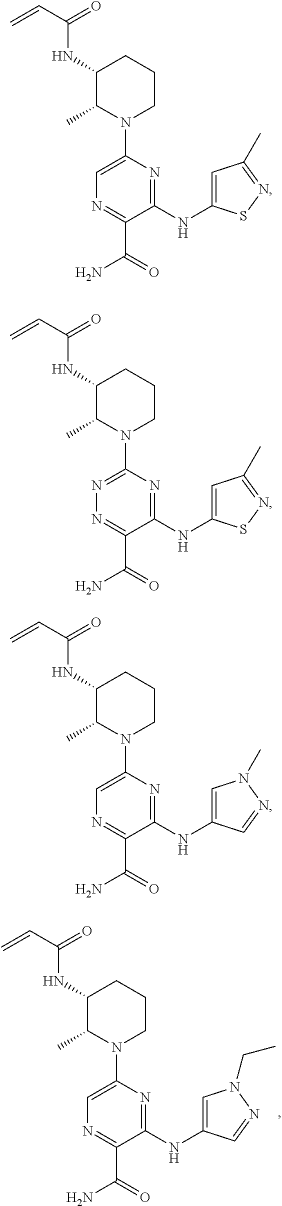

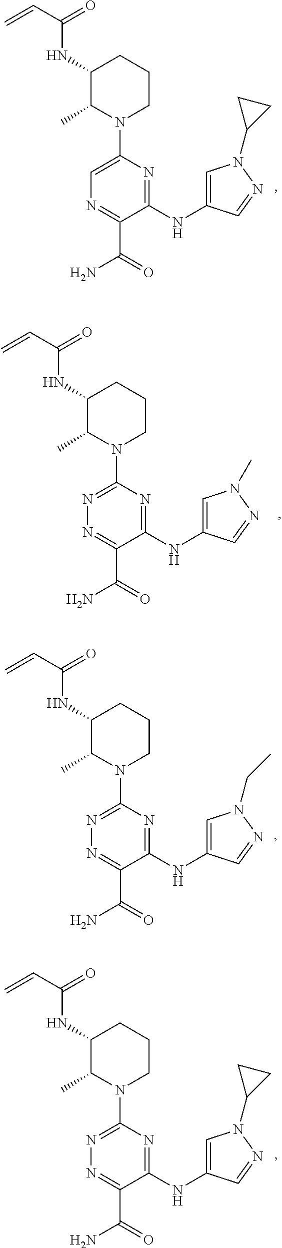

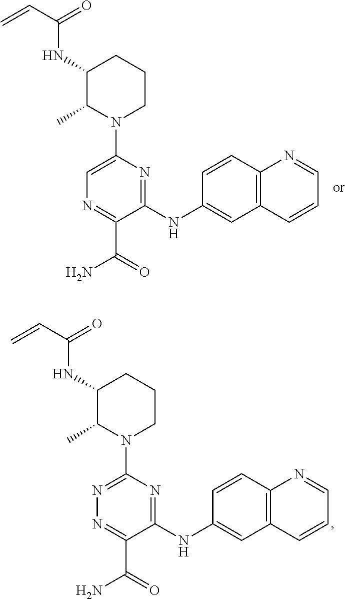 [SMILES: C=CC(=O)N[C@@H]1CCCN(c2cnc(C(N)=O)c(Nc3ccc4ncccc4c3)n2)[C@@H]1C ; C=CC(=O)N[C@@H]1CCCN(c2nnc(C(N)=O)c(Nc3ccc4ncccc4c3)n2)[C@@H]1C]

or a pharmaceutically acceptable solvate, pharmaceutically acceptable salt, and/or pharmaceutically acceptable prodrug thereof.

In some embodiments, the compound is selected from:

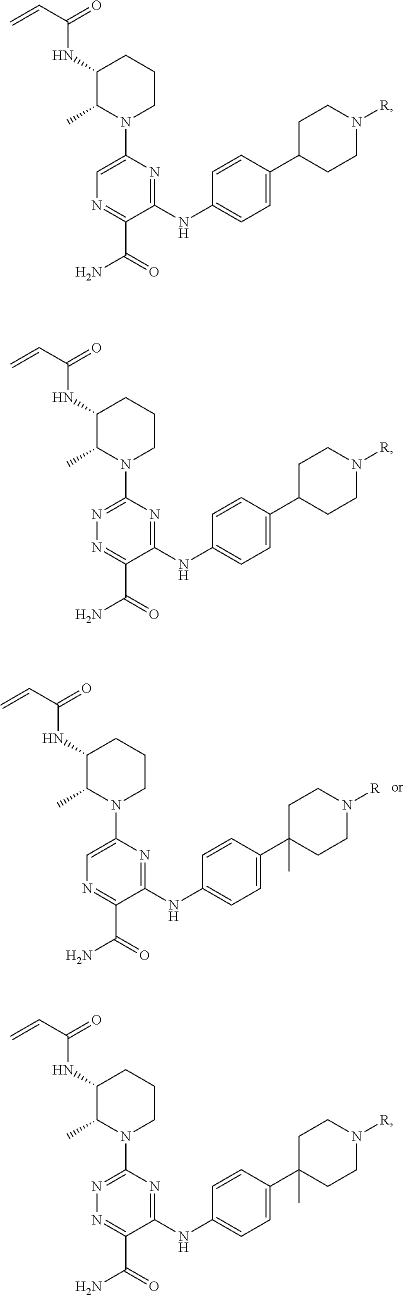

wherein R is methyl, ethyl, isopropyl, cyclopropyl, cyclobutyl, or cyclopentyl, or a pharmaceutically acceptable solvate, pharmaceutically acceptable salt, and/or pharmaceutically acceptable prodrug thereof.

In one aspect, provided herein is a compound of Formula (A-VIII):

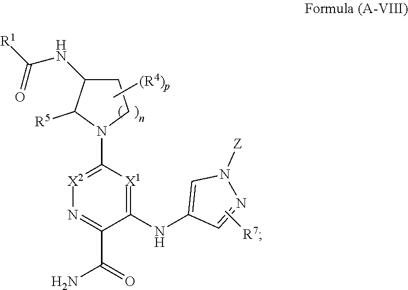

wherein
X¹ and X² are both N or are both C(R²); or X¹ is N and X² is C(R²);
R¹ is bicyclo[1.1.1]pentanyl, phenyl, pyridyl, pyrimidyl or pyrazinyl, wherein the phenyl, pyridyl, pyrimidyl or pyrazinyl is substituted with a substitutent selected from isopropyl, hydroxyl substituted isopropyl, cyano substituted isopropyl, tertbutyl, hydroxyl substituted tertbutyl, cyano substituted tertbutyl, cyclopropyl, fluoro substituted cyclopropy, trifluoromethyl substituted cyclopropyl, oxetanyl, pyridyl, pyrimidyl and dimethylamino, and optionally substituted with trifluoromethyl, fluoro or chloro, or R¹ is CH═CH₂, or

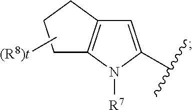

the group

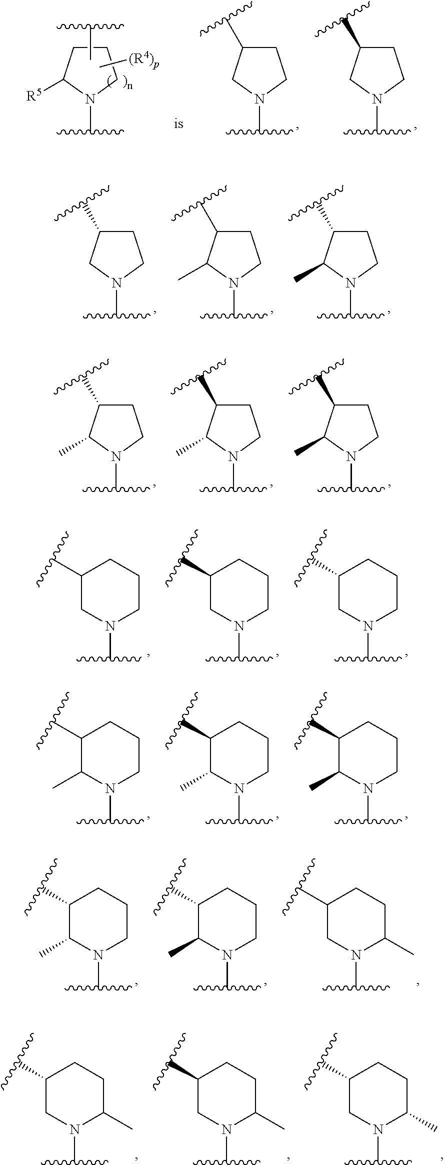

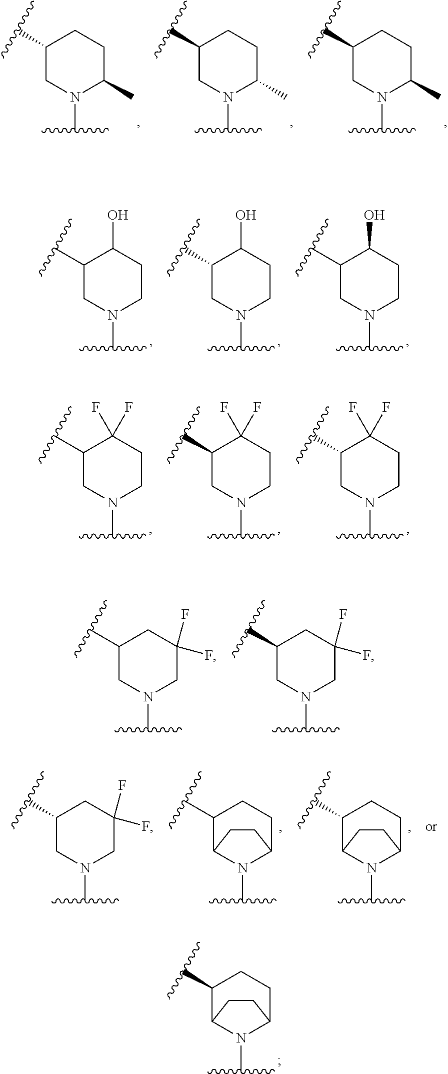

each R⁷ is independently C₁-C₄alkyl, such as methyl, or halo, such as F, Cl, or Br;
each R⁸ is independently H or C₁-C₃alkyl, such as methyl;
Z is H or C₁-C₃alkyl optionally substituted with halo, alkoxy or N(R³⁰)₂ (each R³⁰ is independently H, or substituted or unsubstituted C₁-C₄alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl; and
t is 0, 1 or 2;
or a pharmaceutically acceptable solvate, pharmaceutically acceptable salt, and/or pharmaceutically acceptable prodrug thereof.

In some embodiments, X¹ and X² are both N.

In some embodiments, X¹ and X² are both CH.

In some embodiments, X¹ is N and X² is CH.

In some embodiments, R¹ is phenyl, pyridyl or pyrazinyl, wherein the phenyl, pyridyl or pyrazinyl is substituted with a substitutent selected from isopropyl, tertbutyl, cyclopropyl and dimethylamino, and optionally substituted with fluoro or chloro.

In some embodiments, R¹ is CH═CH₂.

In some embodiments, R¹ is

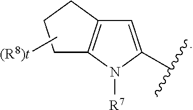

In some embodiments, the compound is:

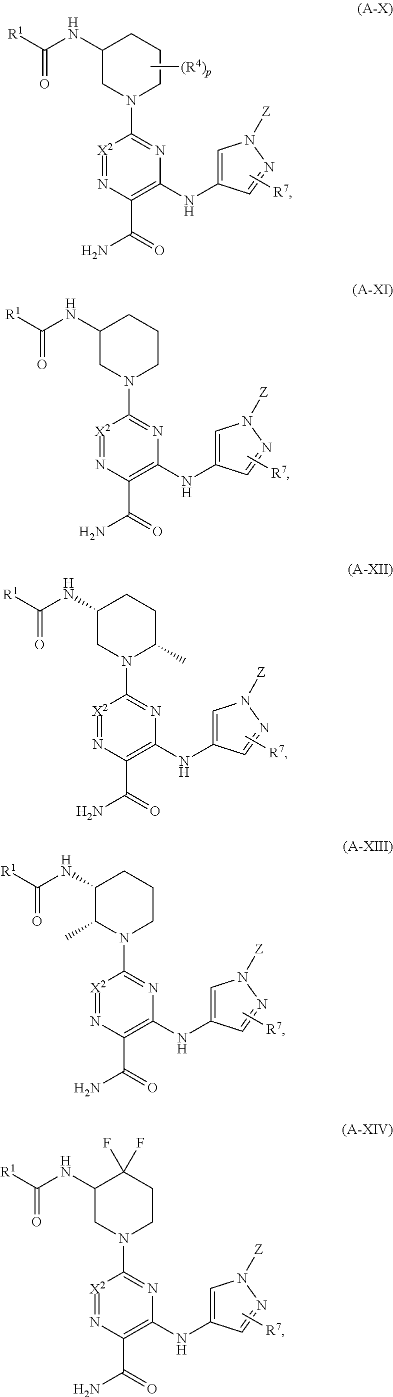

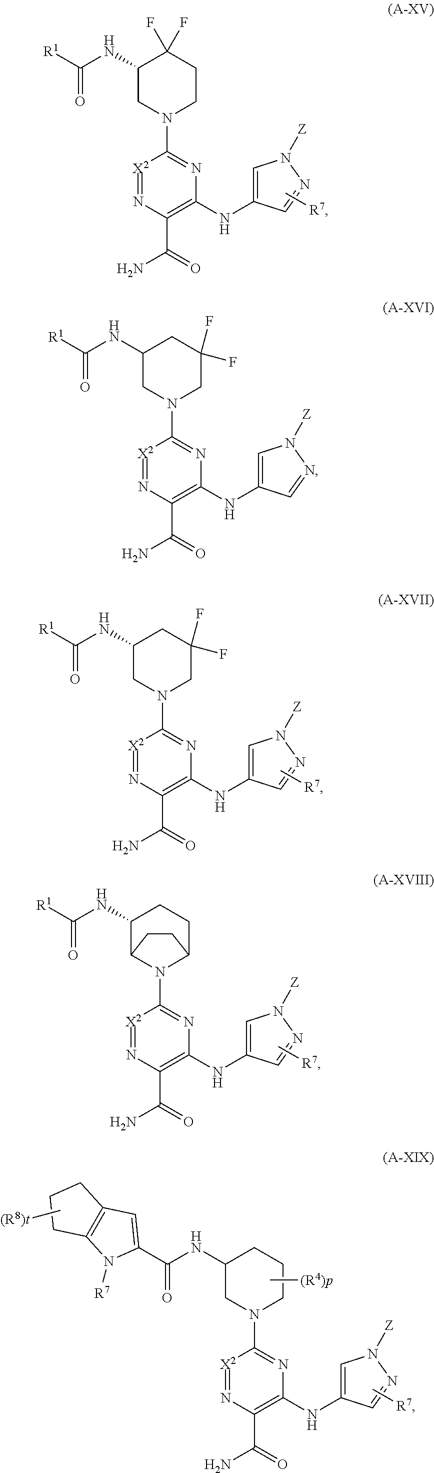

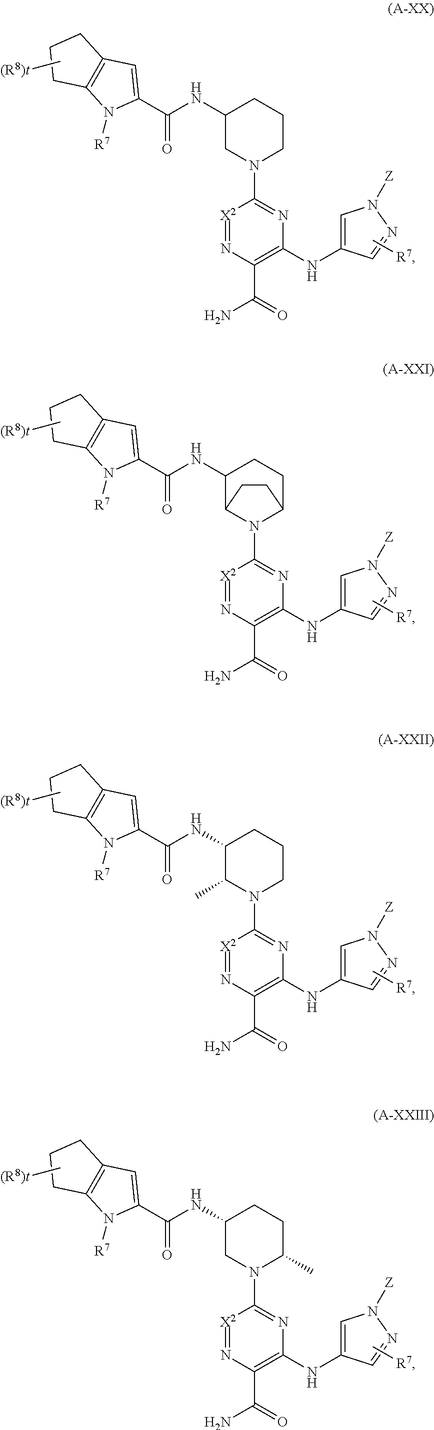

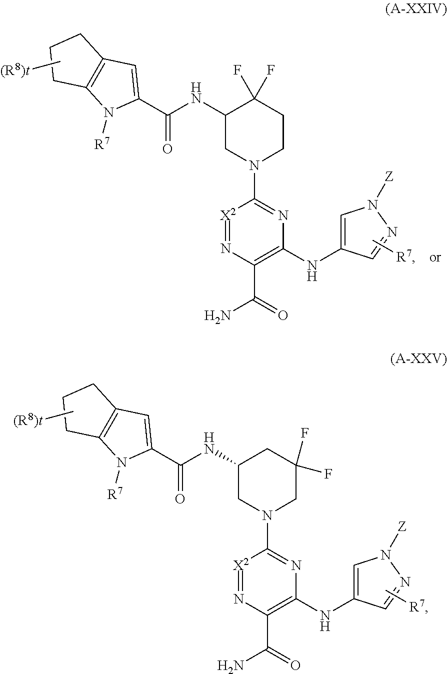

wherein
X² is CH or N,
R¹ is bicyclo[1.1.1]pentanyl, phenyl, pyridyl, pyrimidyl or pyrazinyl, wherein the phenyl, pyridyl, pyrimidyl and pyrazinyl are substituted with a substitutent selected from isopropyl, hydroxyl substituted isopropyl, cyano substituted isopropyl, tertbutyl, hydroxyl substituted tertbutyl, cyano substituted tertbutyl, cyclopropyl, fluoro substituted cyclopropy, trifluoromethyl substituted cyclopropyl, oxetanyl, pyridyl, pyrimidyl and dimethylamino, and optionally substituted with trifluoromethyl, fluoro or chloro,
each R⁴ is independently H, methyl or fluoro,
each R⁷ is independently H or C₁-C₃alkyl, such as methyl,
each R⁸ is independently H or C₁-C₃alkyl, such as methyl,
Z is H or C₁-C₃alkyl optionally substituted with halo, alkoxy or N(R³⁰)₂ (each R³⁰ is independently H, or substituted or unsubstituted C₁-C₄alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl,
p is 0, 1 or 2, and
t is 0, 1 or 2,
or a pharmaceutically acceptable solvate, pharmaceutically acceptable salt, and/or pharmaceutically acceptable prodrug thereof.

In some embodiments,

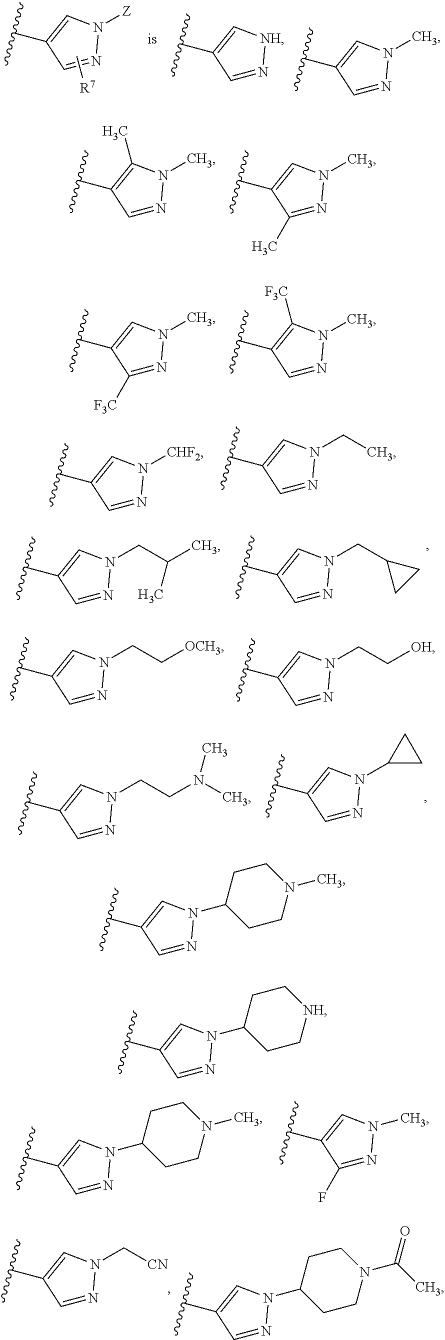

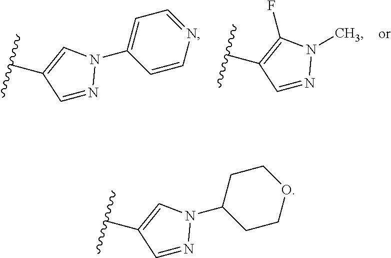

In some embodiments,

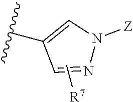

is replaced with

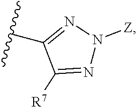

such as

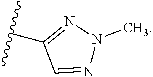 [SMILES: *c1cnn(C)n1]

In some embodiments, R¹ is

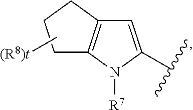

wherein each R⁷ is independently C₁-C₄alkyl, such as methyl, or halo, such as F, Cl, or Br; each R⁸ is independently H or C₁-C₃alkyl, such as methyl; t is 0, 1 or 2.
In some embodiments, R¹ is:

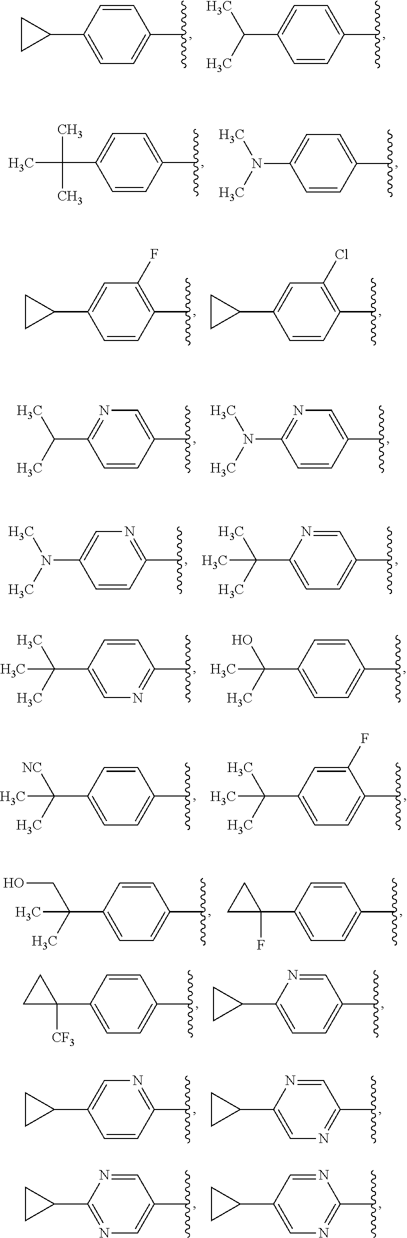

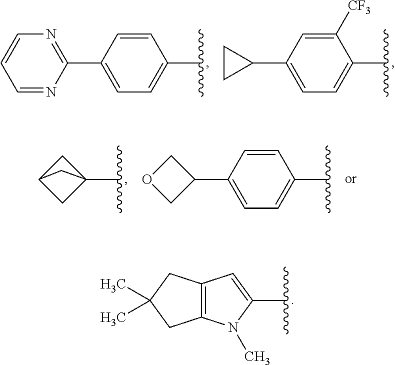

In some embodiments, the compound is a compound of Formula (A-X)-(A-XXV), and X² is CH.

In some embodiments, the compound is a compound according to Formula (A-X)-(A-XXV), and R¹ is phenyl substituted with 1 or 2 groups independently selected from Me, Et, i-Pr, t-Bu, CF₃, CHF₂, cyclopropyl, Cl, F, Br, and CN.

In particular embodiments, the compound is according to Formula (A-X)-(A-XXV), and R¹ is:

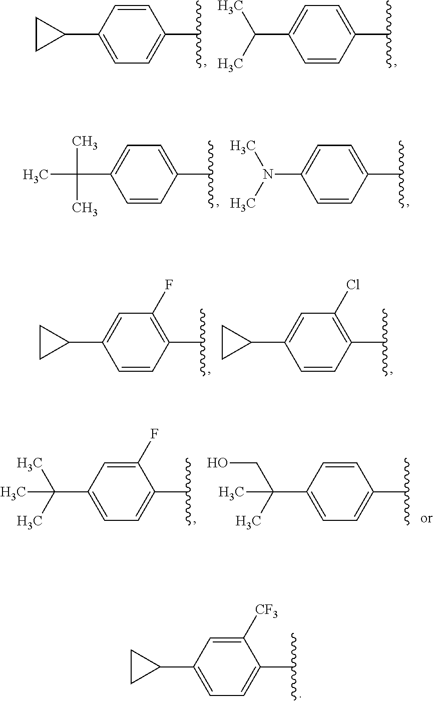

In some embodiments, the compound is:

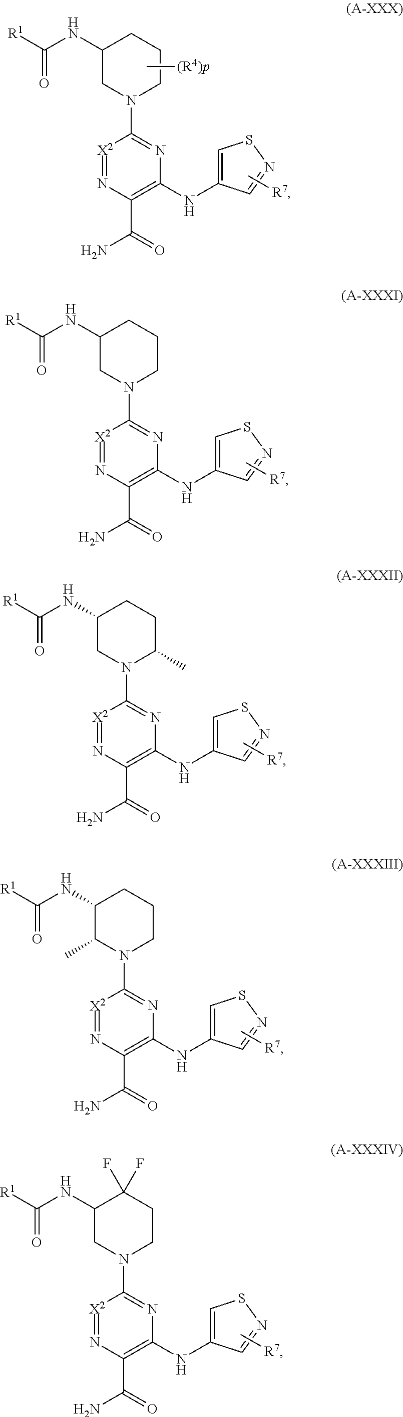

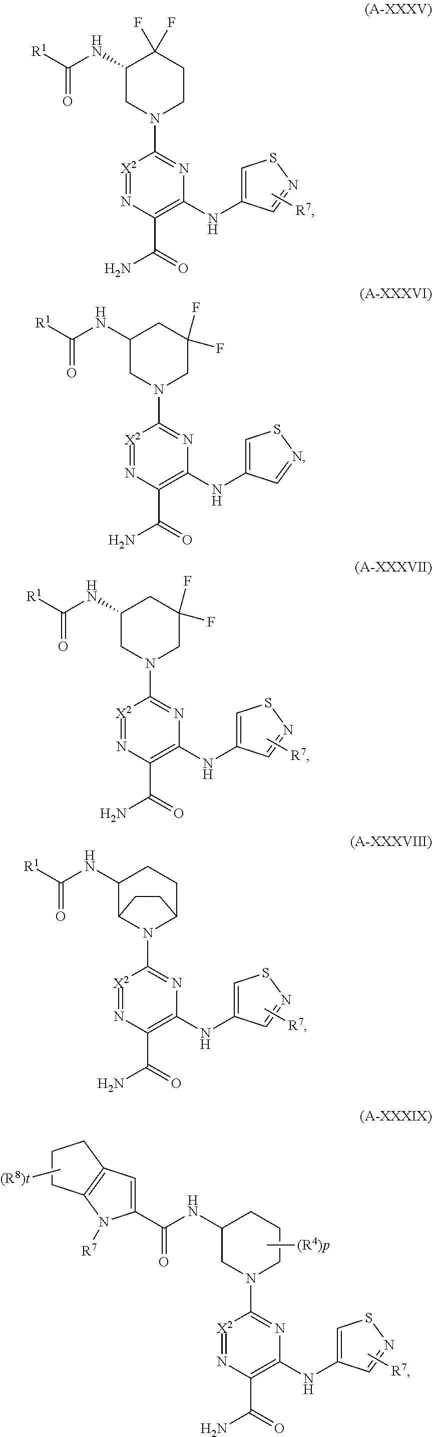

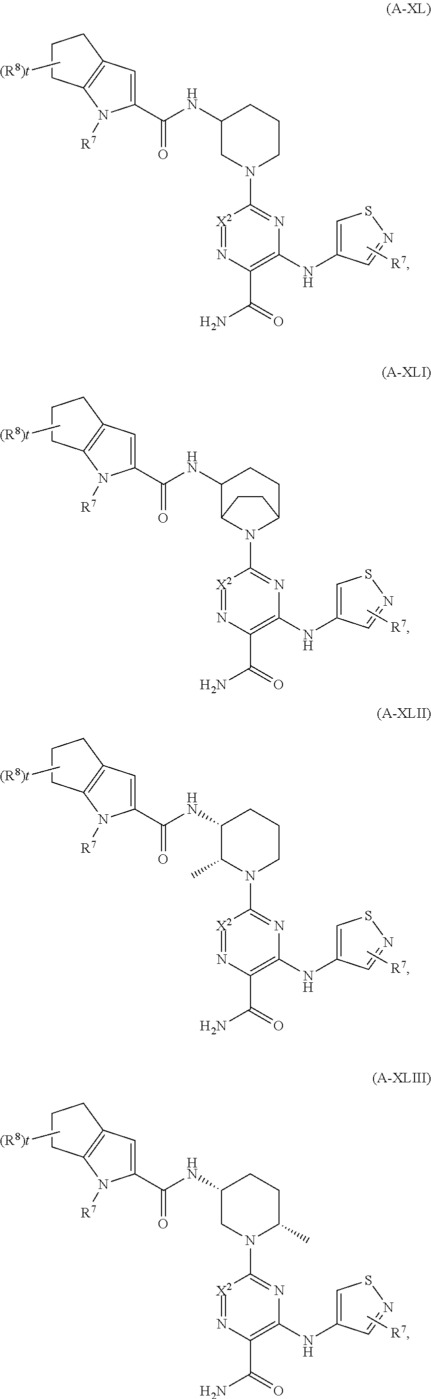

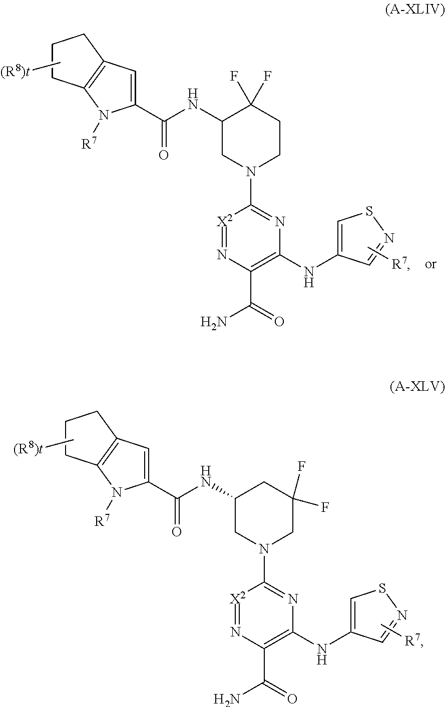

wherein
X² is CH or N,
R¹ is bicyclo[1.1.1]pentanyl, phenyl, pyridyl, pyrimidyl or pyrazinyl, wherein the phenyl, pyridyl, pyrimidyl and pyrazinyl are substituted with a substitutent selected from isopropyl, hydroxyl substituted isopropyl, cyano substituted isopropyl, tertbutyl, hydroxyl substituted tertbutyl, cyano substituted tertbutyl, cyclopropyl, fluoro substituted cyclopropy, trifluoromethyl substituted cyclopropyl, oxetanyl, pyridyl, pyrimidyl and dimethylamino, and optionally substituted with trifluoromethyl, fluoro or chloro,
each R⁴ is independently H, methyl or fluoro,
each R⁷ is independently H or C₁-C₃alkyl, such as methyl,
each R⁸ is independently H or C₁-C₃alkyl, such as methyl,
Z is H or C₁-C₃alkyl optionally substituted with halo, alkoxy or N(R³⁰)₂ (each R³⁰ is independently H, or substituted or unsubstituted C₁-C₄alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl,
p is 0, 1 or 2, and
t is 0, 1 or 2,
or a pharmaceutically acceptable solvate, pharmaceutically acceptable salt, and/or pharmaceutically acceptable prodrug thereof.

In some embodiments, R⁷ is Me, Et, or i-Pr. In another embodiment, R⁷ is 3-Me, 3-Et, or 3-i-Pr.

In some embodiments, R¹ is:

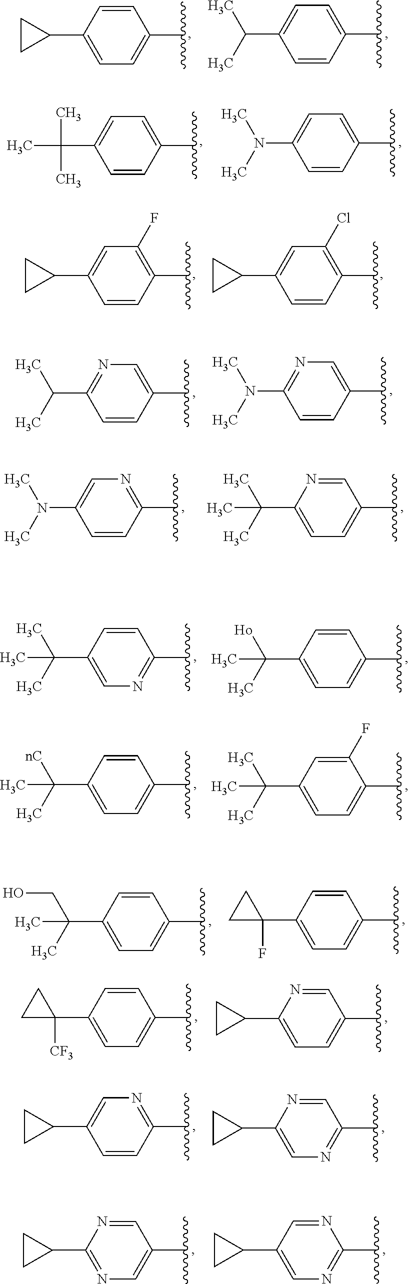

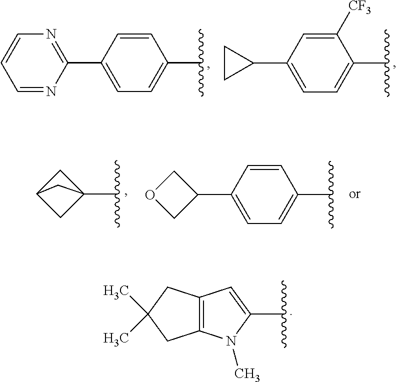

In some embodiments, the compound is:

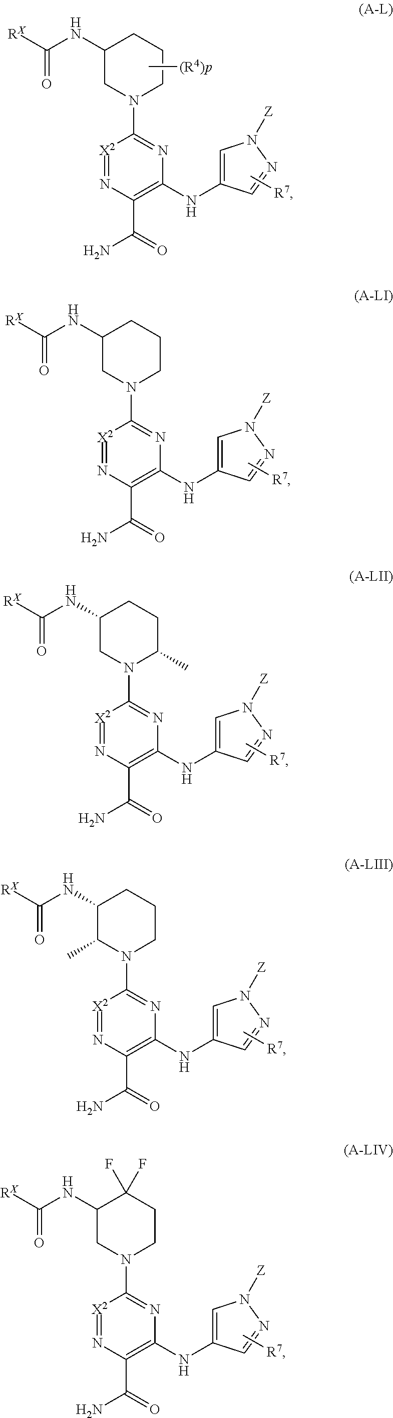

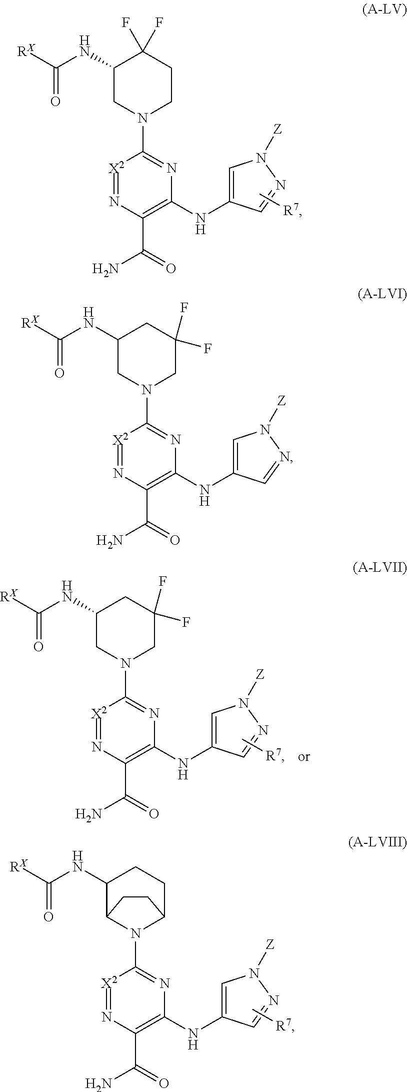

wherein
X² is CH or N,
R^{X} is dialkylamino,
each R⁴ is independently H, methyl or fluoro,
each R⁷ is independently H or C₁-C₃alkyl, such as methyl,
each R⁸ is independently H or C₁-C₃alkyl, such as methyl,
Z is H or C₁-C₃alkyl optionally substituted with halo, alkoxy or N(R³⁰)₂ (each R³⁰ is independently H, or substituted or unsubstituted C₁-C₄alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl,
p is 0, 1 or 2, and
t is 0, 1 or 2,
or a pharmaceutically acceptable solvate, pharmaceutically acceptable salt, and/or pharmaceutically acceptable prodrug thereof.

In some embodiments,

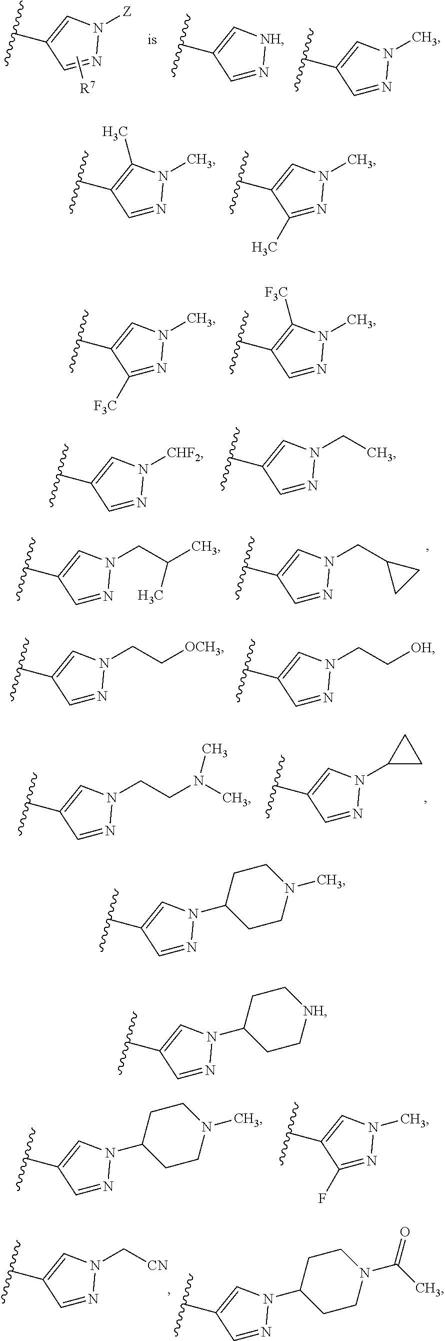

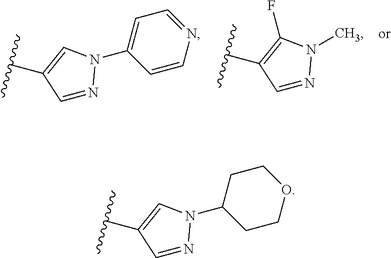

In some embodiments,

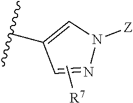

is replaced with

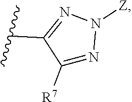

such as

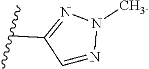 [SMILES: *c1cnn(C)n1]

In some embodiments, R¹ is:

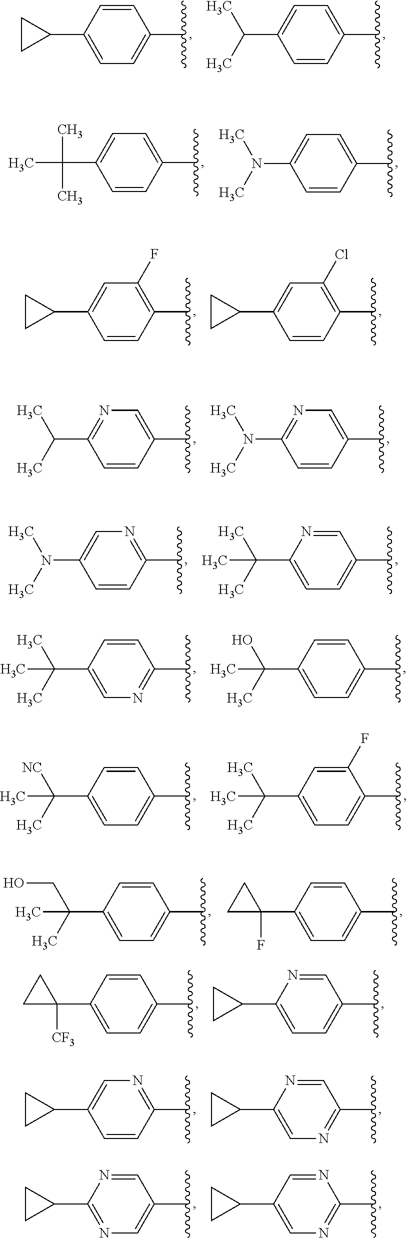

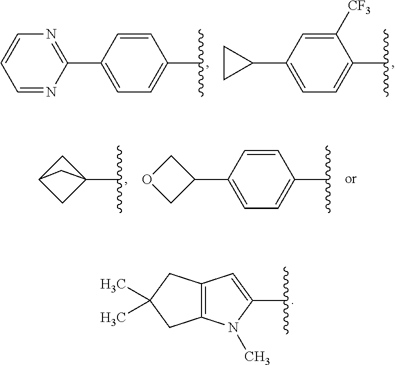

In some embodiments, the compound is according to Formula (A-XXX)-(A-XLV), or (A-LI)-(A-LVIII) and X² is CH.

In some embodiments, the compound is according to Formula (A-XXX)-(A-XLV), or (A-LI)-(A-LVIII) and R¹ is phenyl substituted with 1 or 2 groups independently selected from Me, Et, i-Pr, t-Bu, CF₃, CHF₂, cyclopropyl, Cl, F, Br, and CN.

In particular embodiments, the compound is according to Formula (A-XXX)-(A-XLV), or (A-LI)-(A-LVIII) and R¹ is:

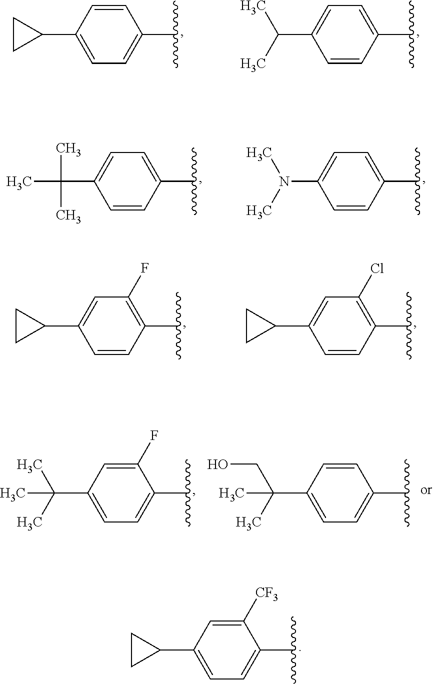

In some embodiments, the compound is:

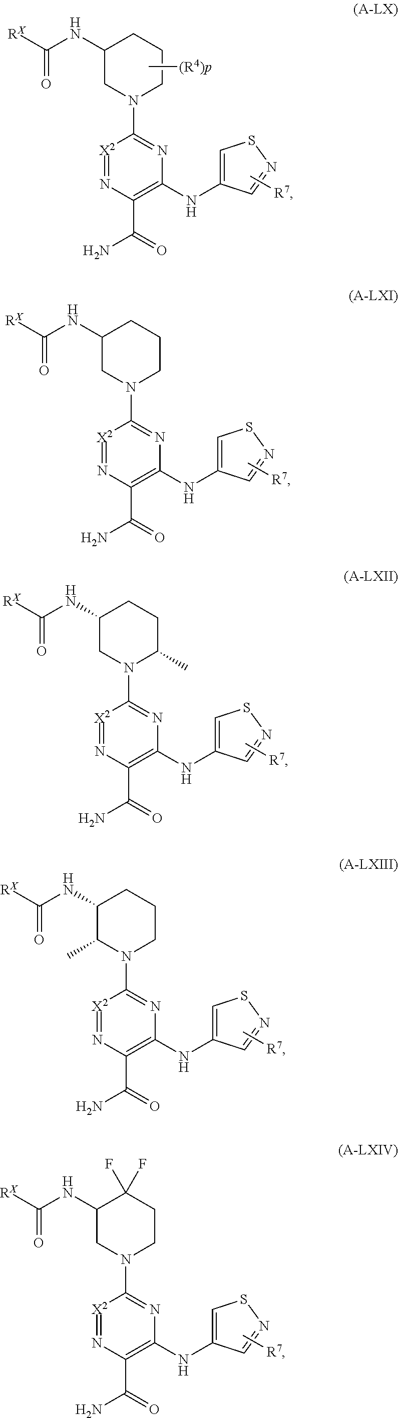

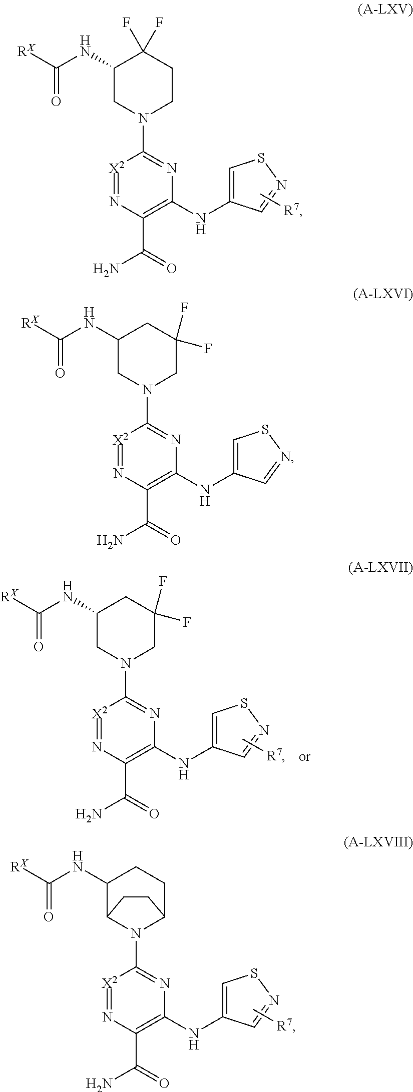

wherein
X² is CH or N,
R^{X} is dialkylamino,
each R⁴ is independently H, methyl or fluoro,
each R⁷ is independently H or C₁-C₃alkyl, such as methyl,
each R⁸ is independently H or C₁-C₃alkyl, such as methyl,
Z is H or C₁-C₃alkyl optionally substituted with halo, alkoxy or N(R³⁰)₂ (each R³⁰ is independently H, or substituted or unsubstituted C₁-C₄alkyl), such as methyl, ethyl, isopropyl, isopropylmethyl, CHF₂, CF₃, 2-methoxyethyl or 2-(dimethylamino)ethyl, or Z is C₃-C₆cycloalkyl or 3- to 6-membered heterocycloalkyl optionally substituted with C₁-C₃alkyl, such as cyclopropyl, 4-methylpiperidiny or tetrahydropyranyl,
p is 0, 1 or 2, and
t is 0, 1 or 2,
or a pharmaceutically acceptable solvate, pharmaceutically acceptable salt, and/or pharmaceutically acceptable prodrug thereof.

In some embodiments, R⁷ is Me, Et, or i-Pr. In another embodiment, R⁷ is 3-Me, 3-Et, or 3-i-Pr.

In some embodiments, R¹ is:

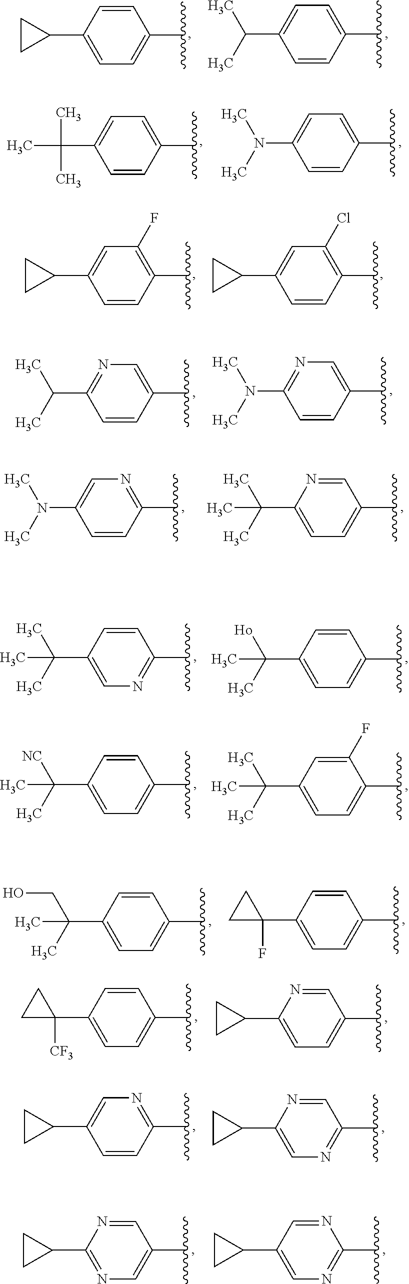

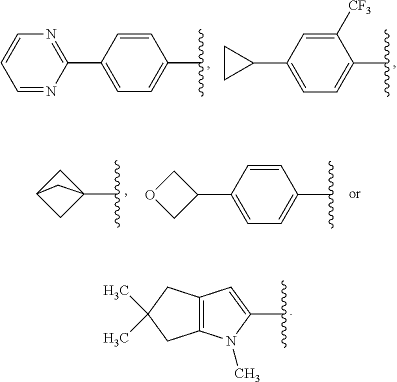

In some embodiments, the compound is according to Formula (A-X)-(A-XXV), (A-XXX)-(A-XLV), (A-L)-(A-LVII), or (A-LX)-(A-LXVIII) and X² is CH.

In some embodiments, the compound is according to Formula (A-X)-(A-XXV), (A-XXX)-(A-XLV), (A-L)-(A-LVII), or (A-LX)-(A-LXVIII) and R¹ is phenyl substituted with 1 or 2 groups independently selected from Me, Et, i-Pr, t-Bu, CF₃, CHF₂, cyclopropyl, Cl, F, Br, and CN.

In particular embodiments, the compound is according to Formula (A-X)-(A-XXV), (A-XXX)-(A-XLV), (A-L)-(A-LVIII), or (A-LX)-(A-LXVIII), and R¹ is:

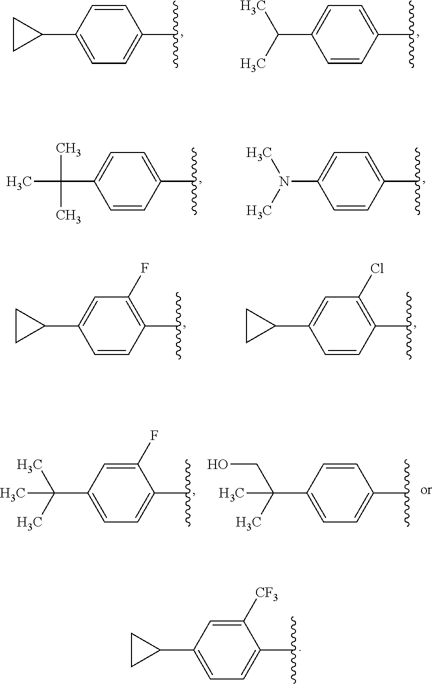

In some embodiments, the compound is selected from:

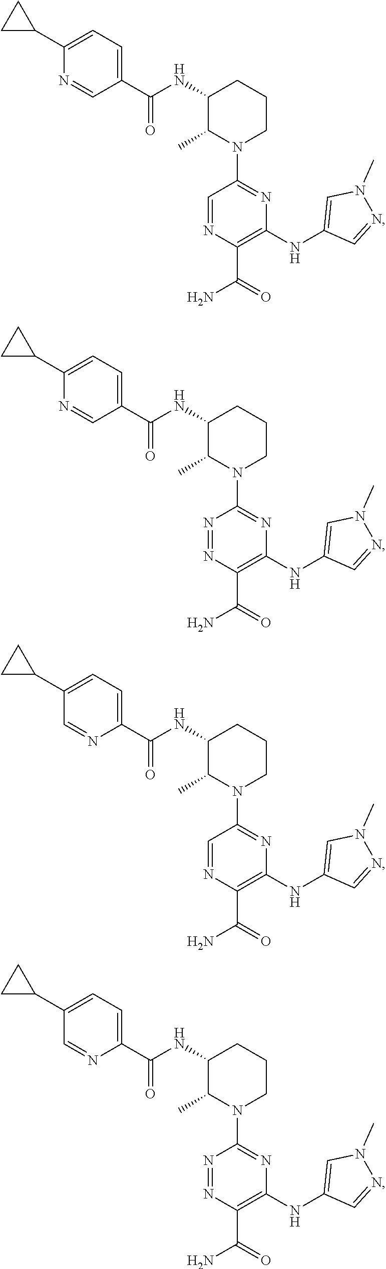 [SMILES: C[C@@H]1[C@H](NC(=O)c2ccc(C3CC3)nc2)CCCN1c1cnc(C(N)=O)c(Nc2cnn(C)c2)n1 ; C[C@@H]1[C@H](NC(=O)c2ccc(C3CC3)nc2)CCCN1c1nnc(C(N)=O)c(Nc2cnn(C)c2)n1 ; C[C@@H]1[C@H](NC(=O)c2ccc(C3CC3)cn2)CCCN1c1cnc(C(N)=O)c(Nc2cnn(C)c2)n1 ; C[C@@H]1[C@H](NC(=O)c2ccc(C3CC3)cn2)CCCN1c1nnc(C(N)=O)c(Nc2cnn(C)c2)n1]

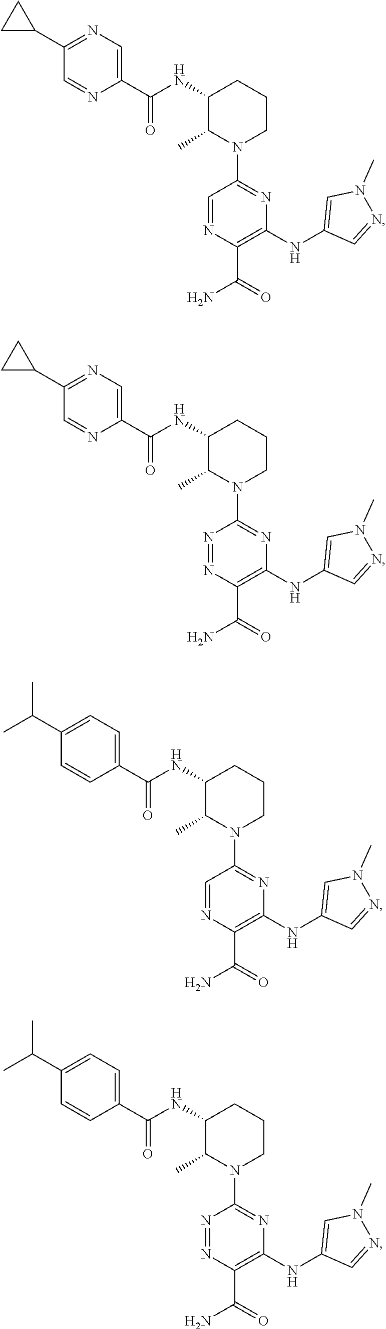 [SMILES: C[C@@H]1[C@H](NC(=O)c2cnc(C3CC3)cn2)CCCN1c1cnc(C(N)=O)c(Nc2cnn(C)c2)n1 ; C[C@@H]1[C@H](NC(=O)c2cnc(C3CC3)cn2)CCCN1c1nnc(C(N)=O)c(Nc2cnn(C)c2)n1 ; CC(C)c1ccc(C(=O)N[C@@H]2CCCN(c3cnc(C(N)=O)c(Nc4cnn(C)c4)n3)[C@@H]2C)cc1 ; CC(C)c1ccc(C(=O)N[C@@H]2CCCN(c3nnc(C(N)=O)c(Nc4cnn(C)c4)n3)[C@@H]2C)cc1]

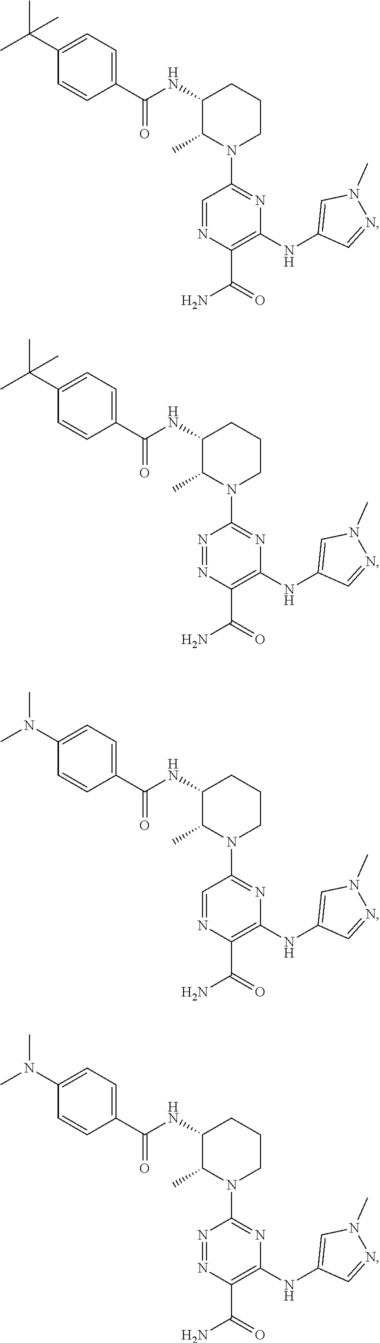 [SMILES: C[C@@H]1[C@H](NC(=O)c2ccc(C(C)(C)C)cc2)CCCN1c1cnc(C(N)=O)c(Nc2cnn(C)c2)n1 ; C[C@@H]1[C@H](NC(=O)c2ccc(C(C)(C)C)cc2)CCCN1c1nnc(C(N)=O)c(Nc2cnn(C)c2)n1 ; C[C@@H]1[C@H](NC(=O)c2ccc(N(C)C)cc2)CCCN1c1cnc(C(N)=O)c(Nc2cnn(C)c2)n1 ; C[C@@H]1[C@H](NC(=O)c2ccc(N(C)C)cc2)CCCN1c1nnc(C(N)=O)c(Nc2cnn(C)c2)n1]

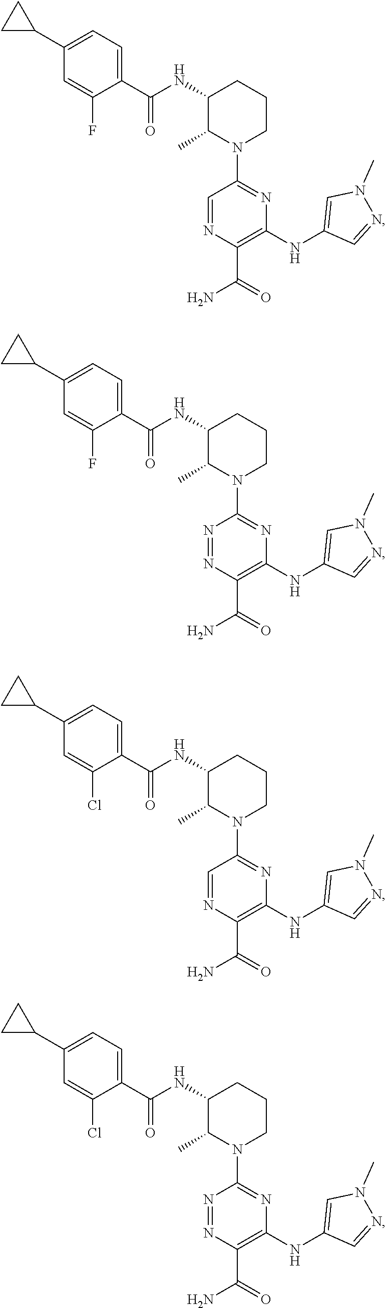 [SMILES: C[C@@H]1[C@H](NC(=O)c2ccc(C3CC3)cc2F)CCCN1c1cnc(C(N)=O)c(Nc2cnn(C)c2)n1 ; C[C@@H]1[C@H](NC(=O)c2ccc(C3CC3)cc2F)CCCN1c1nnc(C(N)=O)c(Nc2cnn(C)c2)n1 ; C[C@@H]1[C@H](NC(=O)c2ccc(C3CC3)cc2Cl)CCCN1c1cnc(C(N)=O)c(Nc2cnn(C)c2)n1 ; C[C@@H]1[C@H](NC(=O)c2ccc(C3CC3)cc2Cl)CCCN1c1nnc(C(N)=O)c(Nc2cnn(C)c2)n1]

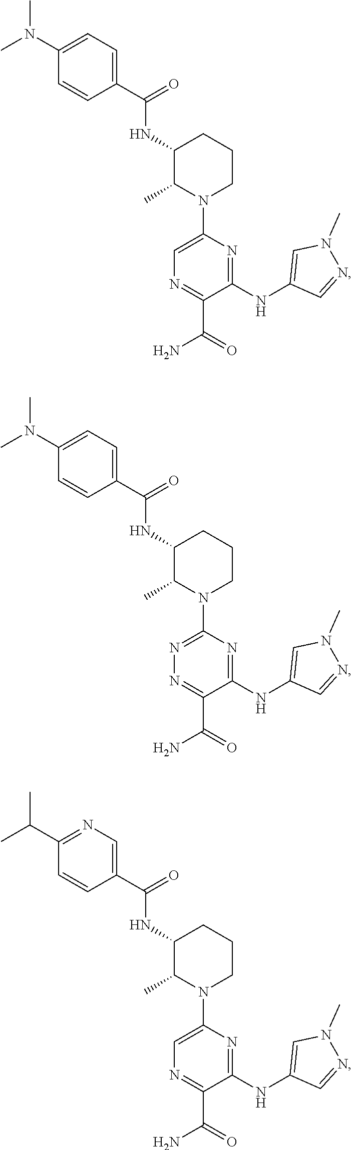 [SMILES: C[C@@H]1[C@H](NC(=O)c2ccc(N(C)C)cc2)CCCN1c1cnc(C(N)=O)c(Nc2cnn(C)c2)n1 ; C[C@@H]1[C@H](NC(=O)c2ccc(N(C)C)cc2)CCCN1c1nnc(C(N)=O)c(Nc2cnn(C)c2)n1 ; CC(C)c1ccc(C(=O)N[C@@H]2CCCN(c3cnc(C(N)=O)c(Nc4cnn(C)c4)n3)[C@@H]2C)cn1]

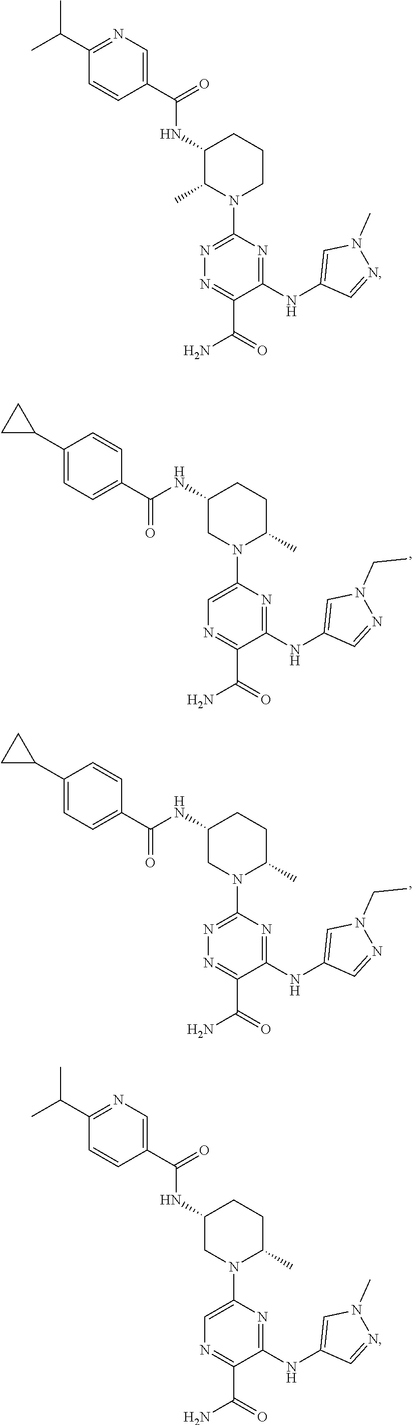

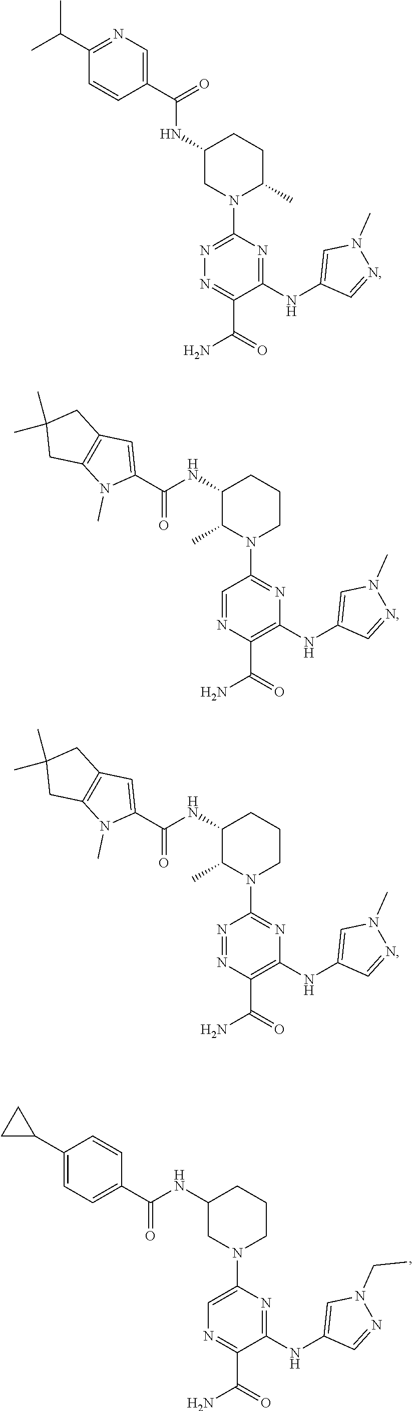 [SMILES: CC(C)c1ccc(C(=O)N[C@@H]2CC[C@H](C)N(c3nnc(C(N)=O)c(Nc4cnn(C)c4)n3)C2)cn1 ; C[C@@H]1[C@H](NC(=O)c2cc3c(n2C)CC(C)(C)C3)CCCN1c1cnc(C(N)=O)c(Nc2cnn(C)c2)n1 ; C[C@@H]1[C@H](NC(=O)c2cc3c(n2C)CC(C)(C)C3)CCCN1c1nnc(C(N)=O)c(Nc2cnn(C)c2)n1 ; CCn1cc(Nc2nc(N3CCCC(NC(=O)c4ccc(C5CC5)cc4)C3)cnc2C(N)=O)cn1]

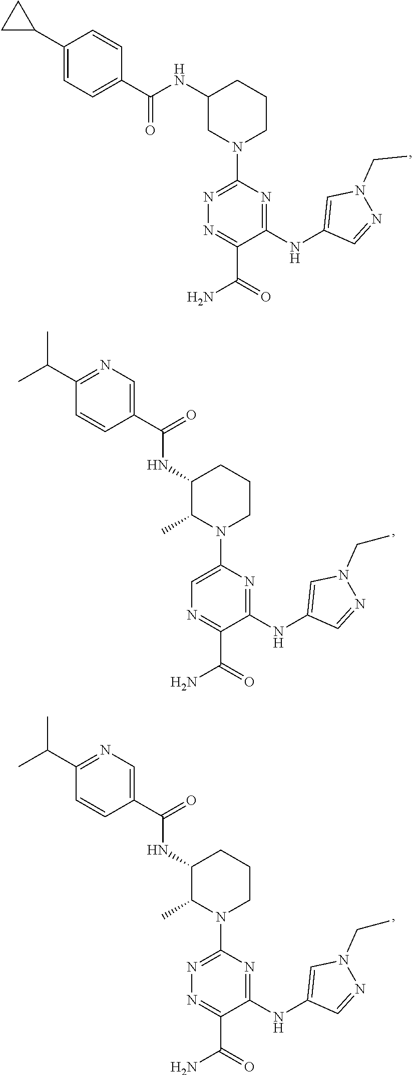

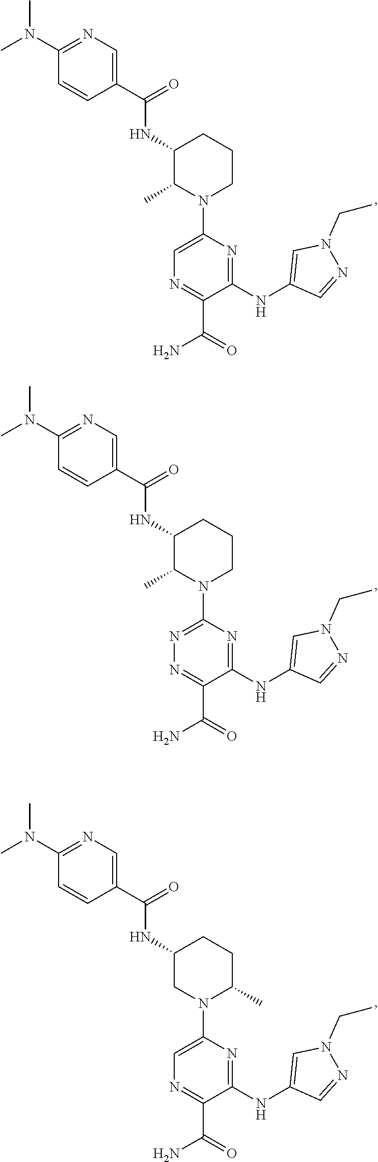

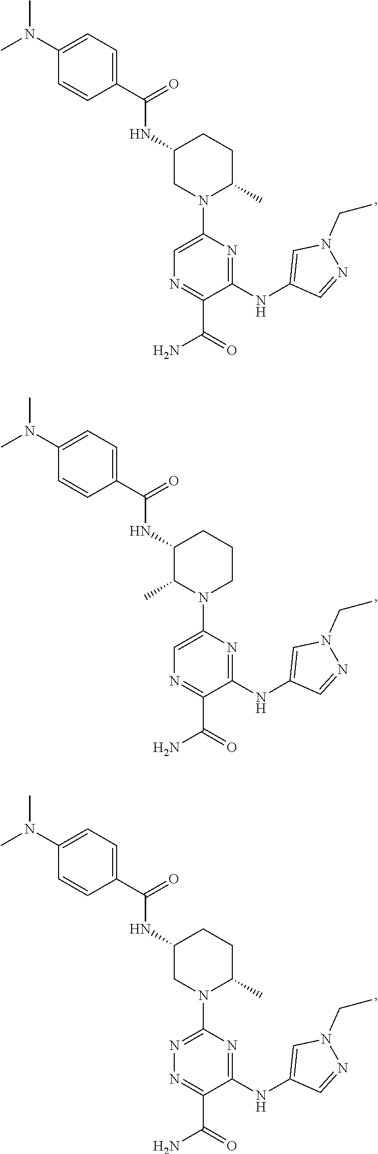

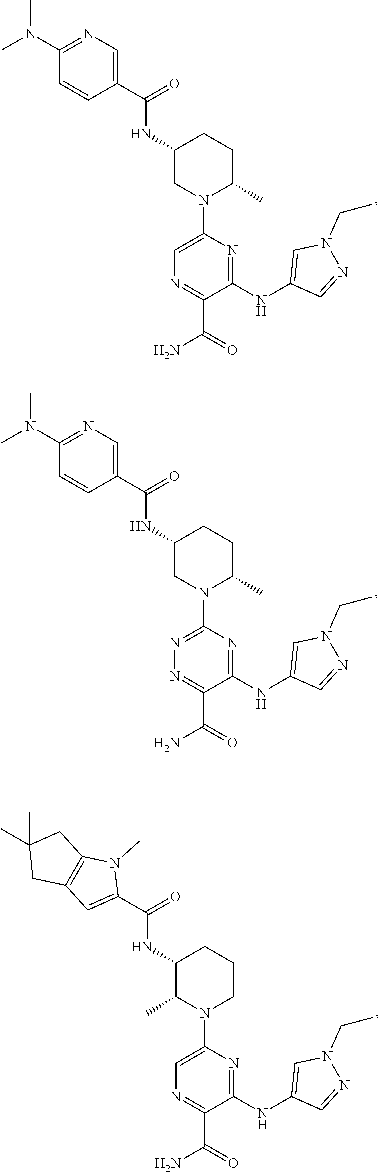

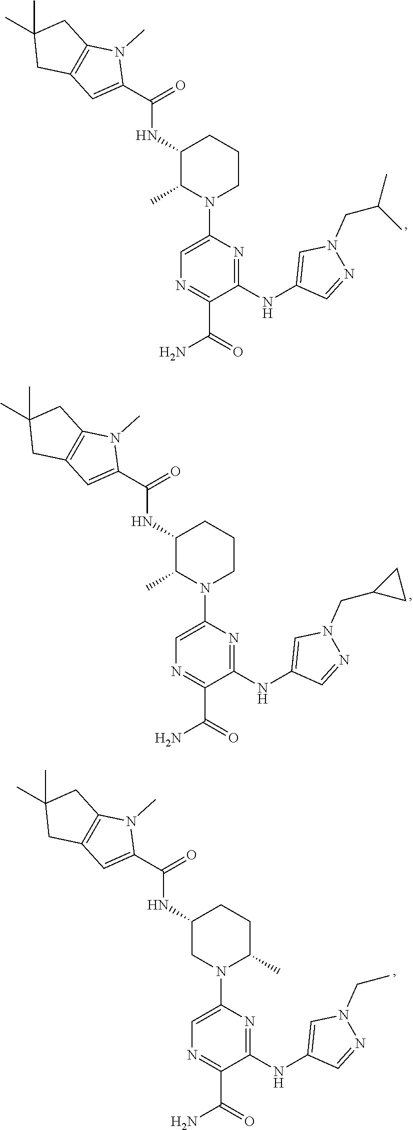

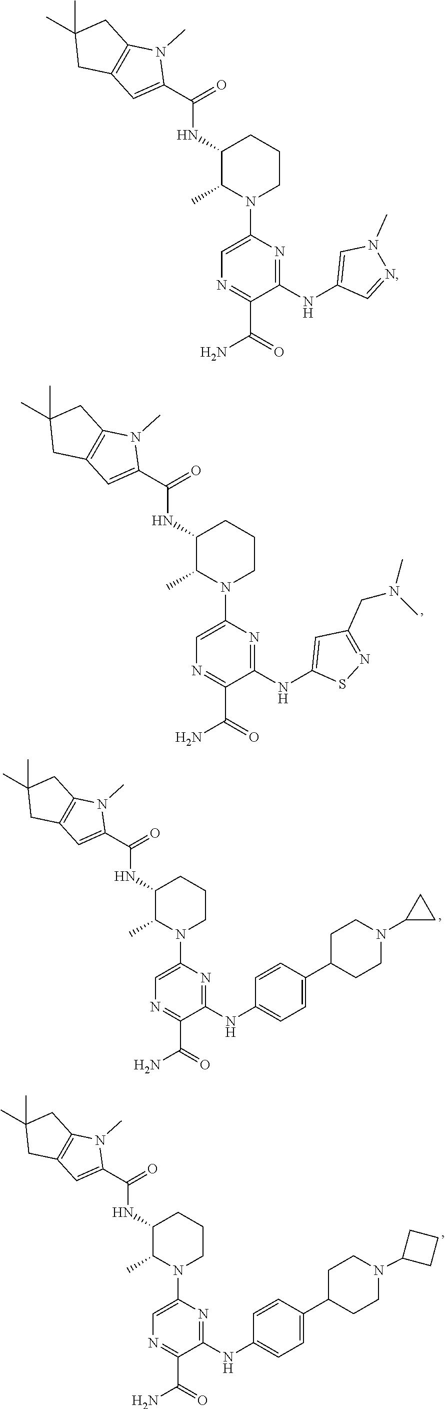

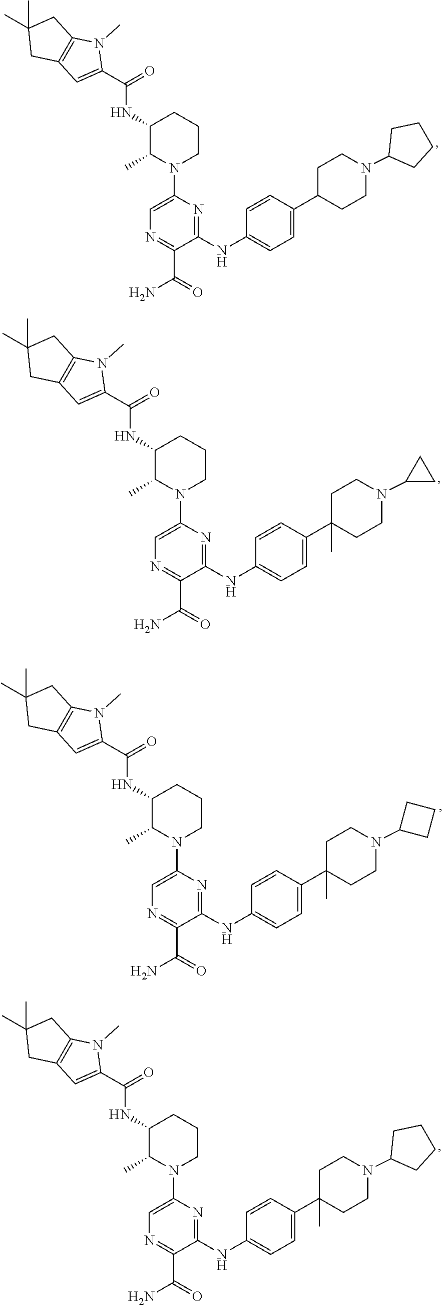 [SMILES: C[C@@H]1[C@H](NC(=O)c2cc3c(n2C)CC(C)(C)C3)CCCN1c1cnc(C(N)=O)c(Nc2ccc(C3CCN(C4CCCC4)CC3)cc2)n1 ; C[C@@H]1[C@H](NC(=O)c2cc3c(n2C)CC(C)(C)C3)CCCN1c1cnc(C(N)=O)c(Nc2ccc(C3(C)CCN(C4CC4)CC3)cc2)n1 ; C[C@@H]1[C@H](NC(=O)c2cc3c(n2C)CC(C)(C)C3)CCCN1c1cnc(C(N)=O)c(Nc2ccc(C3(C)CCN(C4CCC4)CC3)cc2)n1 ; C[C@@H]1[C@H](NC(=O)c2cc3c(n2C)CC(C)(C)C3)CCCN1c1cnc(C(N)=O)c(Nc2ccc(C3(C)CCN(C4CCCC4)CC3)cc2)n1]

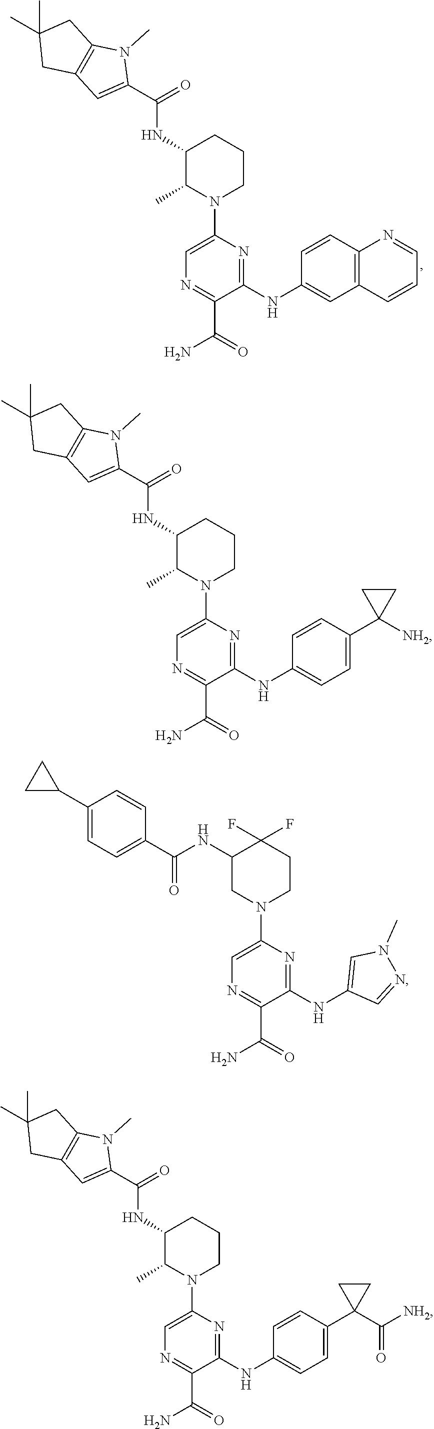 [SMILES: C[C@@H]1[C@H](NC(=O)c2cc3c(n2C)CC(C)(C)C3)CCCN1c1cnc(C(N)=O)c(Nc2ccc3ncccc3c2)n1 ; C[C@@H]1[C@H](NC(=O)c2cc3c(n2C)CC(C)(C)C3)CCCN1c1cnc(C(N)=O)c(Nc2ccc(C3(N)CC3)cc2)n1 ; Cn1cc(Nc2nc(N3CCC(F)(F)C(NC(=O)c4ccc(C5CC5)cc4)C3)cnc2C(N)=O)cn1 ; C[C@@H]1[C@H](NC(=O)c2cc3c(n2C)CC(C)(C)C3)CCCN1c1cnc(C(N)=O)c(Nc2ccc(C3(C(N)=O)CC3)cc2)n1]

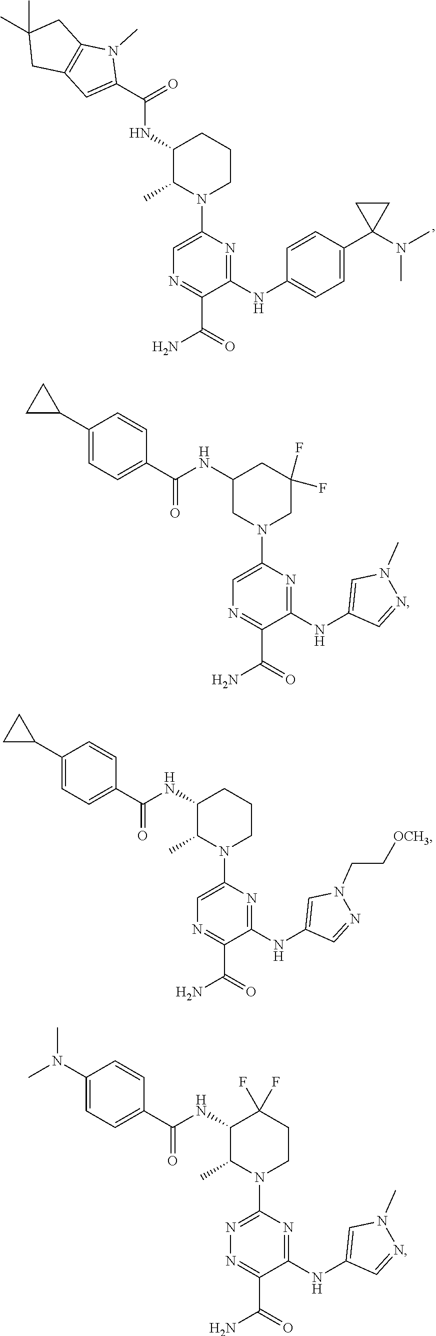

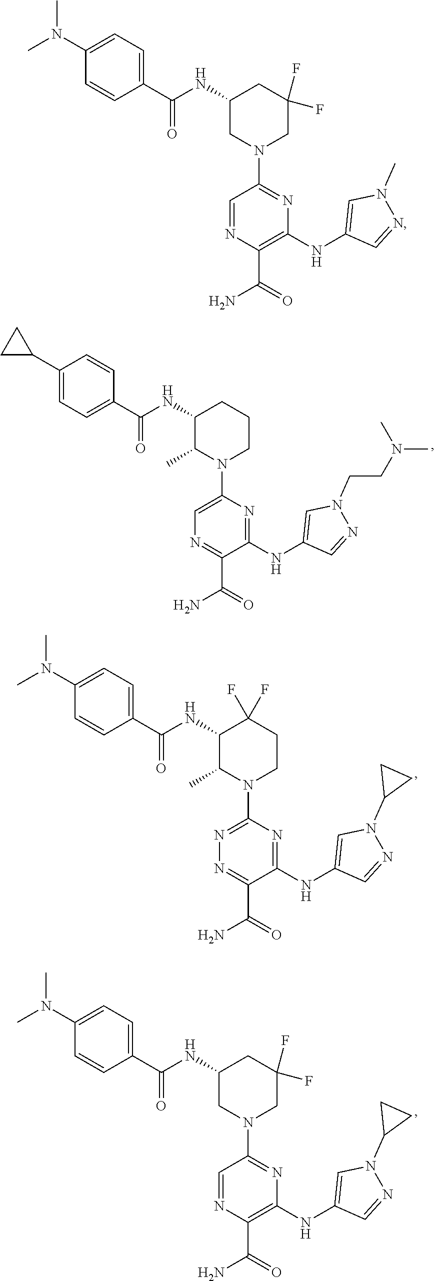

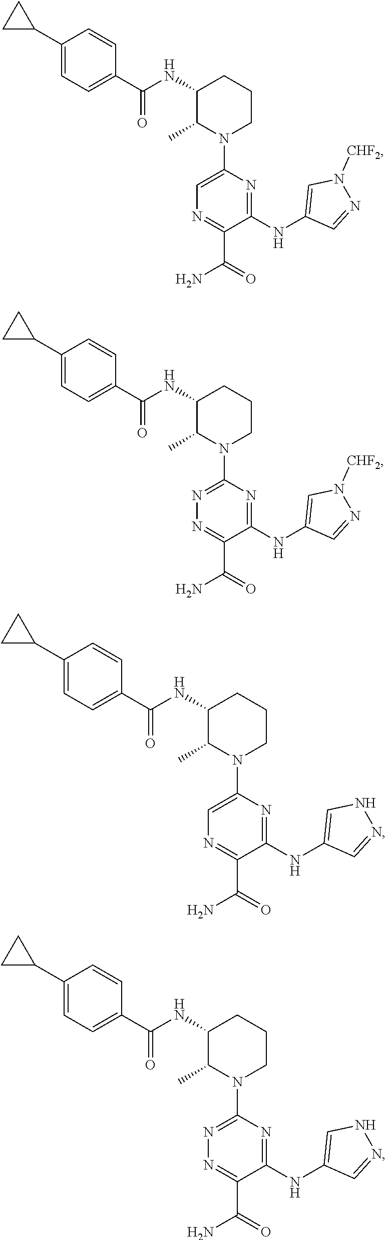 [SMILES: C[C@@H]1[C@H](NC(=O)c2ccc(C3CC3)cc2)CCCN1c1cnc(C(N)=O)c(Nc2cnn(C(F)F)c2)n1 ; C[C@@H]1[C@H](NC(=O)c2ccc(C3CC3)cc2)CCCN1c1nnc(C(N)=O)c(Nc2cnn(C(F)F)c2)n1 ; C[C@@H]1[C@H](NC(=O)c2ccc(C3CC3)cc2)CCCN1c1cnc(C(N)=O)c(Nc2cn[nH]c2)n1 ; C[C@@H]1[C@H](NC(=O)c2ccc(C3CC3)cc2)CCCN1c1nnc(C(N)=O)c(Nc2cn[nH]c2)n1]

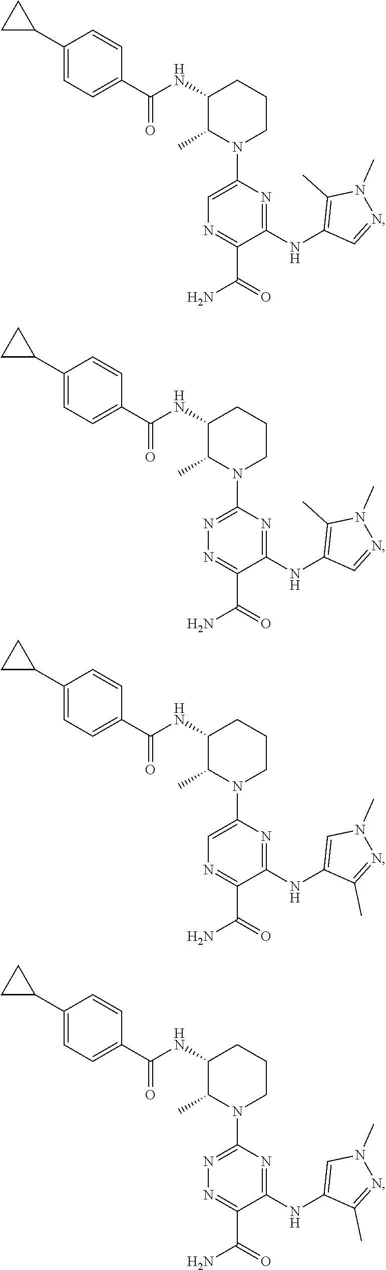 [SMILES: Cc1c(Nc2nc(N3CCC[C@@H](NC(=O)c4ccc(C5CC5)cc4)[C@H]3C)cnc2C(N)=O)cnn1C ; Cc1c(Nc2nc(N3CCC[C@@H](NC(=O)c4ccc(C5CC5)cc4)[C@H]3C)nnc2C(N)=O)cnn1C ; Cc1nn(C)cc1Nc1nc(N2CCC[C@@H](NC(=O)c3ccc(C4CC4)cc3)[C@H]2C)cnc1C(N)=O ; Cc1nn(C)cc1Nc1nc(N2CCC[C@@H](NC(=O)c3ccc(C4CC4)cc3)[C@H]2C)nnc1C(N)=O]

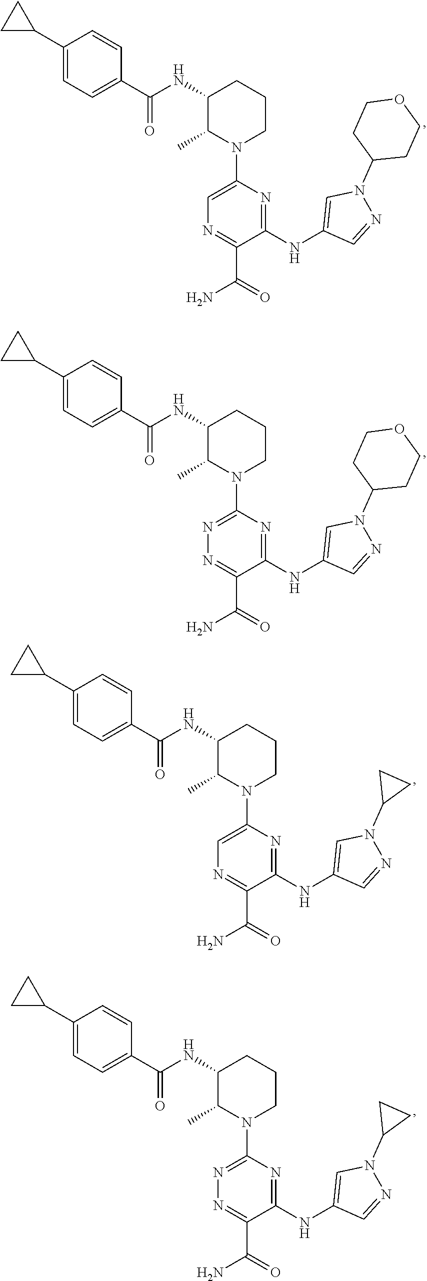 [SMILES: C[C@@H]1[C@H](NC(=O)c2ccc(C3CC3)cc2)CCCN1c1cnc(C(N)=O)c(Nc2cnn(C3CCOCC3)c2)n1 ; C[C@@H]1[C@H](NC(=O)c2ccc(C3CC3)cc2)CCCN1c1nnc(C(N)=O)c(Nc2cnn(C3CCOCC3)c2)n1 ; C[C@@H]1[C@H](NC(=O)c2ccc(C3CC3)cc2)CCCN1c1cnc(C(N)=O)c(Nc2cnn(C3CC3)c2)n1 ; C[C@@H]1[C@H](NC(=O)c2ccc(C3CC3)cc2)CCCN1c1nnc(C(N)=O)c(Nc2cnn(C3CC3)c2)n1]

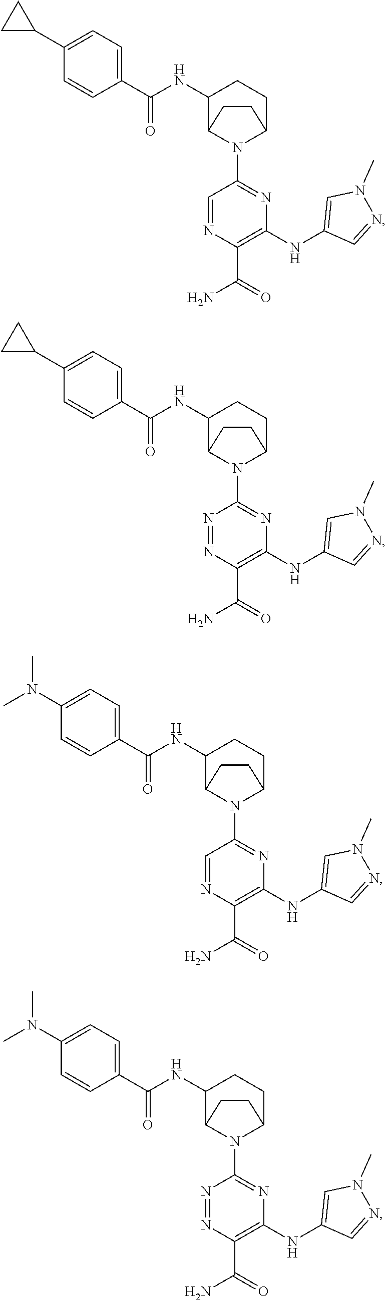 [SMILES: Cn1cc(Nc2nc(N3C4CCC(NC(=O)c5ccc(C6CC6)cc5)C3CC4)cnc2C(N)=O)cn1 ; Cn1cc(Nc2nc(N3C4CCC(NC(=O)c5ccc(C6CC6)cc5)C3CC4)nnc2C(N)=O)cn1 ; CN(C)c1ccc(C(=O)NC2CCC3CCC2N3c2cnc(C(N)=O)c(Nc3cnn(C)c3)n2)cc1 ; CN(C)c1ccc(C(=O)NC2CCC3CCC2N3c2nnc(C(N)=O)c(Nc3cnn(C)c3)n2)cc1]

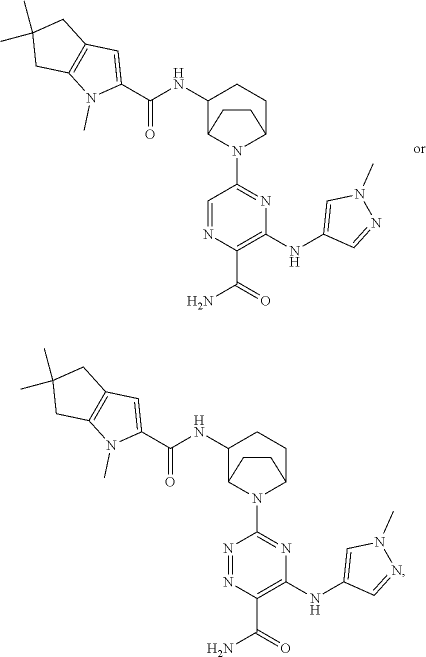 [SMILES: Cn1cc(Nc2nc(N3C4CCC(NC(=O)c5cc6c(n5C)CC(C)(C)C6)C3CC4)cnc2C(N)=O)cn1 ; Cn1cc(Nc2nc(N3C4CCC(NC(=O)c5cc6c(n5C)CC(C)(C)C6)C3CC4)nnc2C(N)=O)cn1]

or a stereoisomer, pharmaceutically acceptable solvate, pharmaceutically acceptable salt, and/or pharmaceutically acceptable prodrug thereof.

In another aspect, provided herein is a compound of Formula (B-ma), (B-IIIb), (B-IIIc), (B-IIId), (B-IIIe), (B-IVa), (B-IVb), (B-IVc), (B-IVd), (B-IVe) or (B-VIII), having the structure:

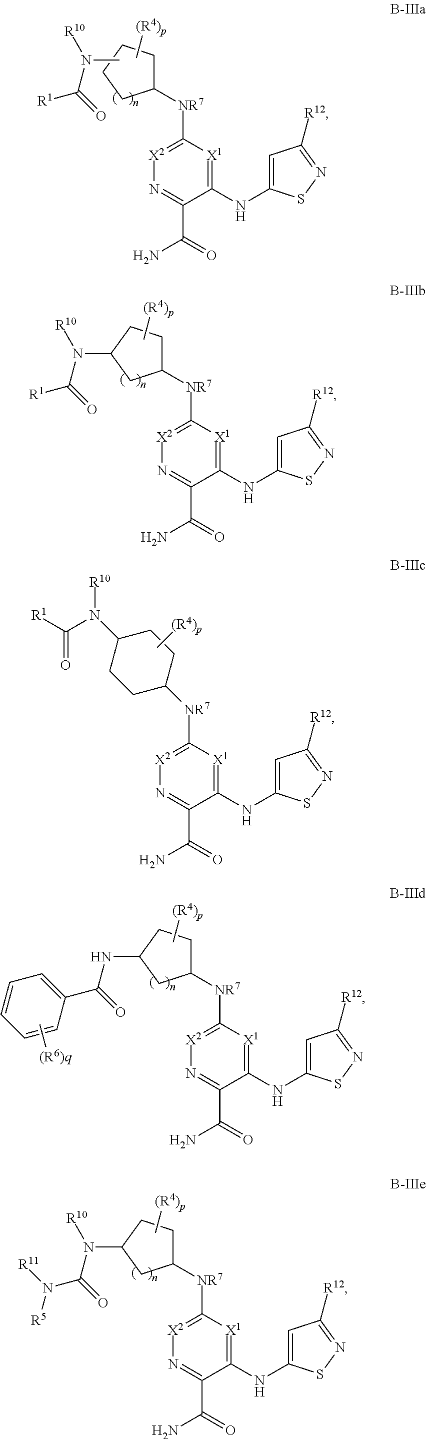

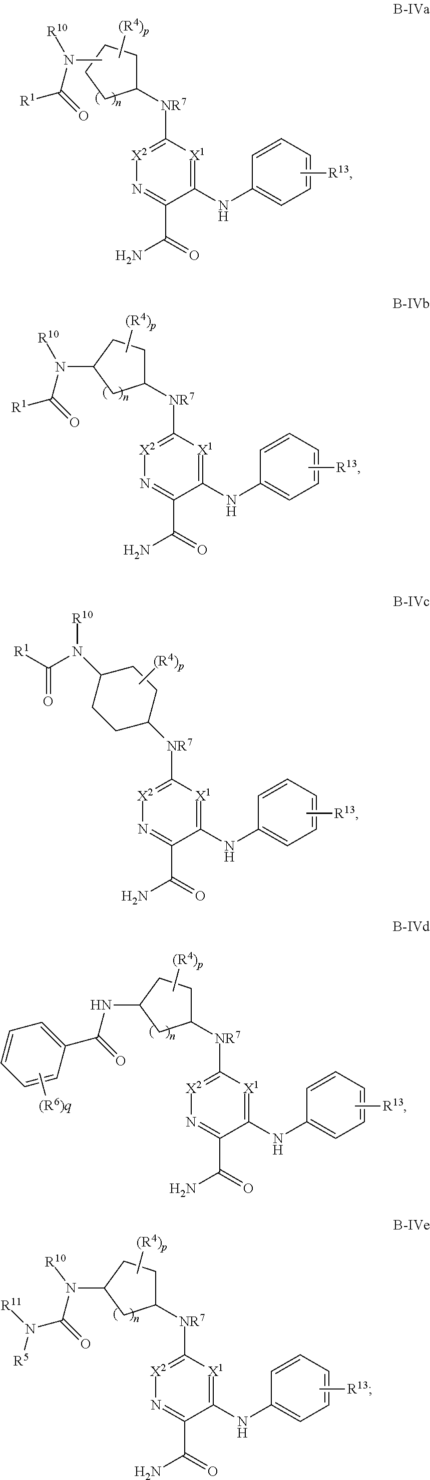

wherein:
X¹, X², R¹, R⁴, R⁵, R⁶, R⁷, R¹⁰, R¹¹, n, p and q are as defined herein;
R¹² is substituted or unsubstituted C₁-C₃alkyl, and
R¹³ is substituted or unsubstituted C₂-C₆heterocycloalkyl,
or a pharmaceutically acceptable solvate, pharmaceutically acceptable salt, or pharmaceutically acceptable prodrug thereof.

In some embodiments, R¹² is unsubstituted C₁-C₃alkyl, such as methyl or ethyl.

In some embodiments, R¹³ is C₂-C₆heterocycloalkyl substituted with C₃-C₇cycloalkyl, such as cyclopropyl, cyclobutyl, cyclopentyl, cyclohexyl or cycloheptyl, and optionally substituted with C₁-C₃alkyl. In some embodiments, R¹³ is

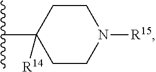

wherein R¹⁴ is hydrogen or C₁-C₃alkyl, such as methyl, and R¹⁵ is hydrogen, C₁-C₆alkyl or C₃-C₈cycloalkyl. In some embodiments, R¹³ is not substituted or unsubstituted piperazine.

In further embodiments the compound is a compound of Formula (B-I), (B-II), (B-IA), (B-IB), (B-IIa)-(B-IId), (B-IIIa)-(B-IIIe), (B-IVa)-(B-IVe) or (B-VIII) wherein X¹ and X² are both N. In further embodiments the compound is a compound of Formula (B-I), (B-II), (B-IA), (B-IB), (B-IIa)-(B-IId), (B-IIa)-(B-IIIe), (B-IVa)-(B-IVe) or (B-VIII) wherein X¹ and X² are independently C(R²). In some embodiments, X¹ and X² are both CH. In further embodiments the compound is a compound of Formula (B-I), (B-II), (B-IA), (B-IB), (B-IIa)-(B-IId), (B-IIIa)-(B-IIIe), (B-IVa)-(B-IVe) or (B-VIII) wherein X¹ is N and X² is C(R²). In some embodiments, X¹ is N and X² is CH. In some embodiments, X¹ and X² are both N or are both CH or X¹ is —N— and X² is CH. In further embodiments the compound is a compound of Formula (B-I), (B-II), (B-IA), (B-IIa)-(B-IId), (B-IIIa), (B-IIb), (B-IId), (B-IIIe), (B-IVa), (B-IVb), (B-IVd), (B-IVe) or (B-VIII) wherein n is 0. In further embodiments the compound is a compound of Formula (B-I), (B-II), (B-IA), (B-IIa)-(B-IId), (B-ma), (B-IIIb), (B-IIId), (B-IIIe), (B-IVa), (B-IVb), (B-IVd), (B-IVe) or (B-VIII) wherein n is 1. In further embodiments the compound is a compound of Formula (B-I), (B-II), (B-IA), (B-IIa)-(B-IId), (B-IIa), (B-IIIb), (B-IIId), (B-IIIe), (B-IVa), (B-IVb), (B-IVd), (B-IVe) or (B-VIII) wherein n is 2. In further embodiments the compound is a compound of Formula (B-I), (B-II), (B-IA), (B-IIa)-(B-IId), (B-IIIa), (B-IIIb), (B-IIId), (B-IIIe), (B-IVa), (B-IVb), (B-IVd), (B-IVe) or (B-VIII) wherein X¹ is N and n is 1. In further embodiments the compound is a compound of Formula (B-I), (B-II), (B-IA), (B-IIa)-(B-IId), (B-IIIa), (B-IIIb), (B-IIId), (B-IIIe), (B-IVa), (B-IVb), (B-IVd), (B-IVe) or (B-VIII) wherein X¹ is N and n is 2. In further embodiments the compound is a compound of Formula (B-I), (B-II), (B-IA), (B-IIa)-(B-IId), (B-IIIa), (B-IIIb), (B-IIId), (B-IIIe), (B-IVa), (B-IVb), (B-IVd), (B-IVe) or (B-VII) wherein X² is C(R²), in particular CH, and n is 1. In further embodiments the compound is a compound of Formula (B-I), (B-II), (B-IA), (B-IIa)-(B-IId), (B-IIIa), (B-IIIb), (B-IIId), (B-IIIe), (B-IVa), (B-IVb), (B-IVd), (B-IVe) or (B-VIII) wherein X² is C(R²), in particular CH, and n is 2. In some embodiments, each R² is independently H, substituted or unsubstituted C₁-C₄alkyl, —CN, or halogen.

In some embodiments, the present invention provides is a compound of Formula (B-Ha), (B-IIc), (B-IIId), or (B-IVd), q is 1. In some embodiments, q is 2 or 3. In some embodiments, q is 2 or 3. In another embodiment is a compound of Formula (B-IIa), (B-IIc), (B-IId), or (B-IVd), wherein each R⁶ is independently Me, Et, i-Pr, cyclopropyl, cyclobutyl, cyclopentyl, Cl, F, amino, or dimethylamino. In some embodiments, each R⁶ is independently —F, CF₃, OCH₃, or OCF₃. In some embodiments, at least one R⁶ is —N(R³)₂ or —OH. In some embodiments, at least one R⁶ is —N(R³)₂. In some embodiments, at least one R⁶ is —NH₂. In some embodiments, at least one R⁶ is —N(CH₃)₂. In some embodiments, at least one R⁶ is —OH.

In some embodiments, the compound is:

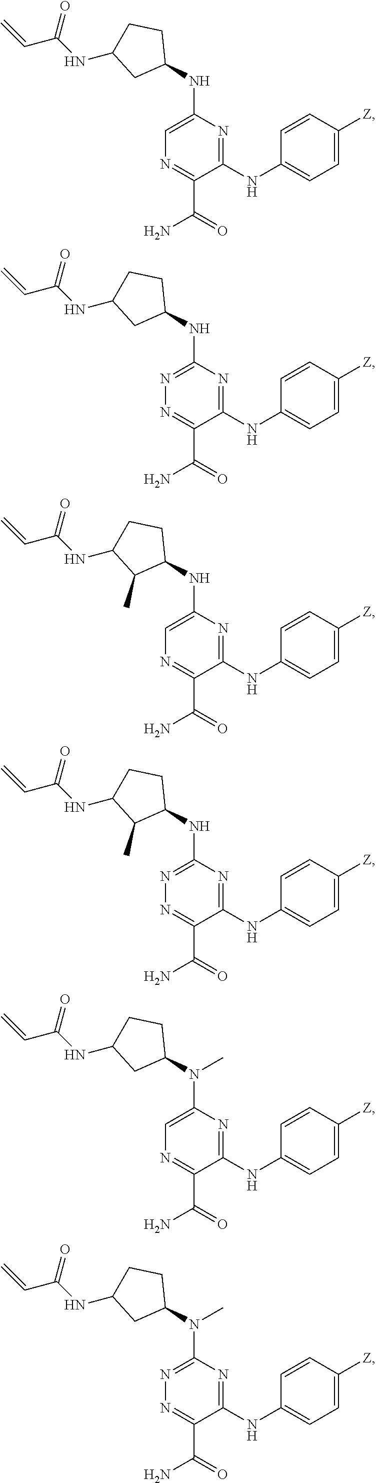

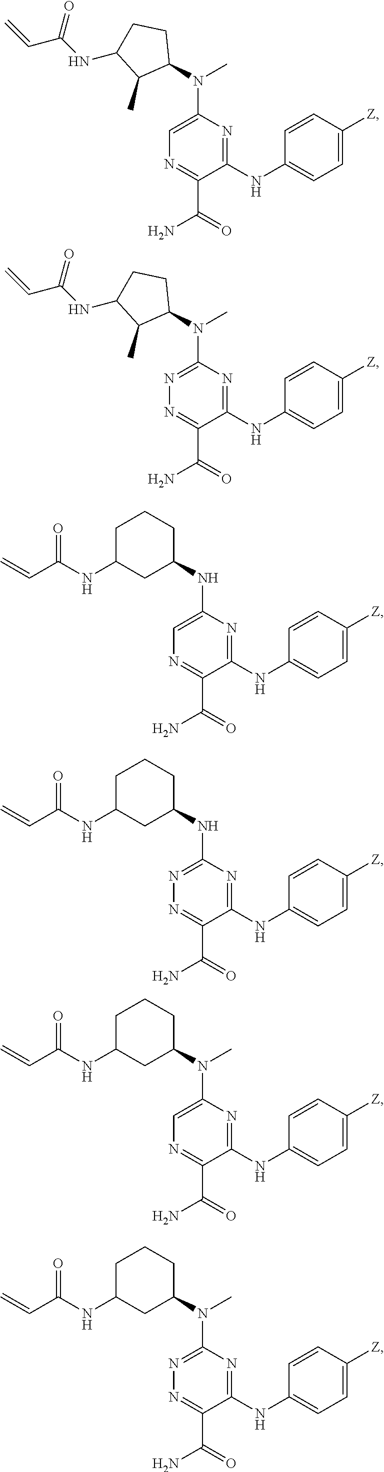

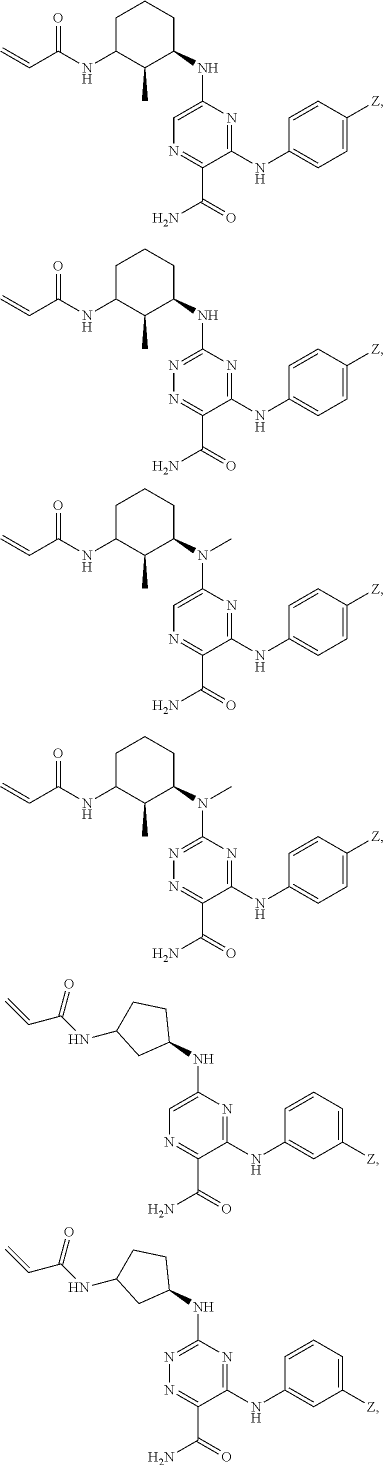

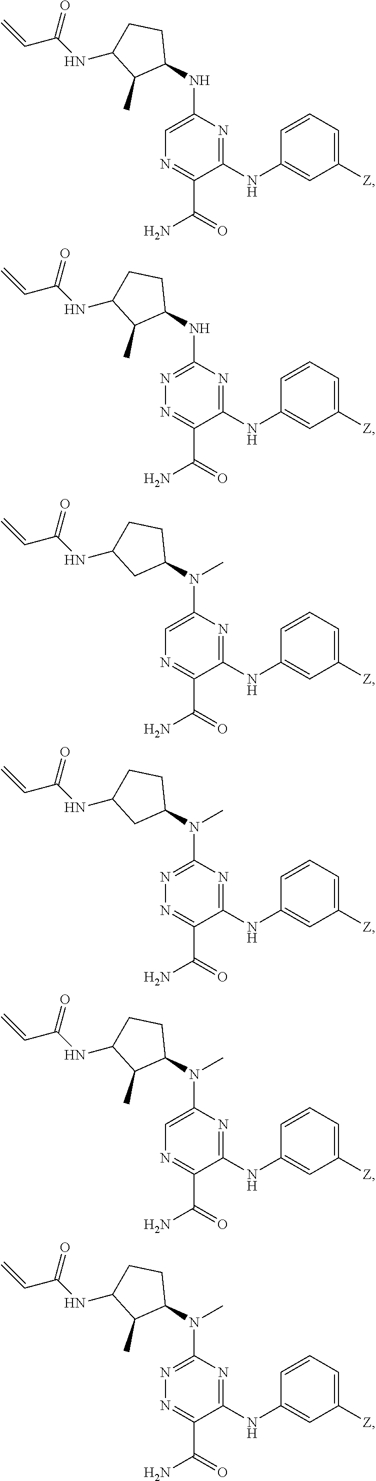

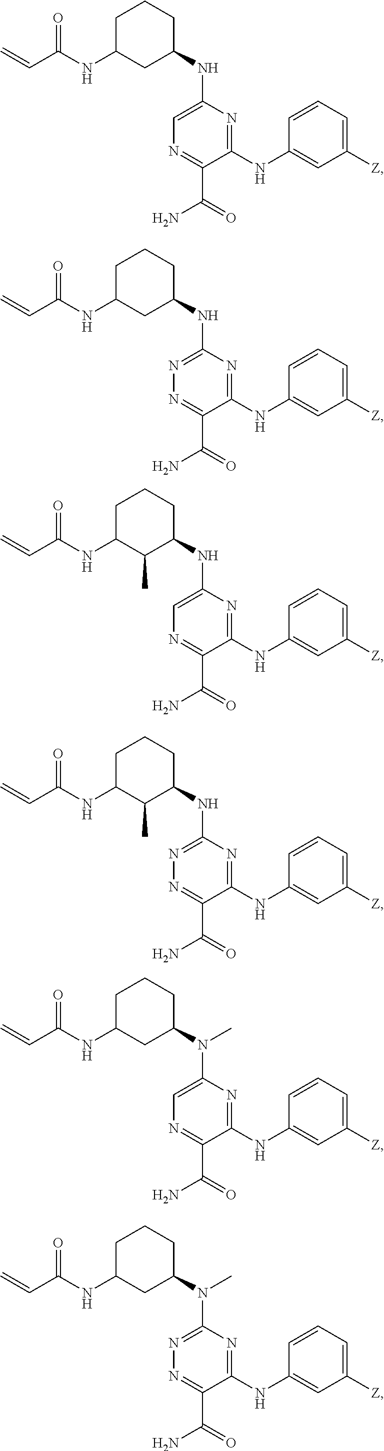

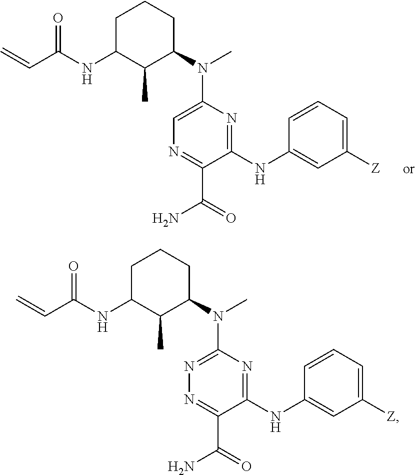

wherein Z is a 5- or 6-membered heteroaryl optionally substituted with methyl, ethyl or hydoxyl, such as

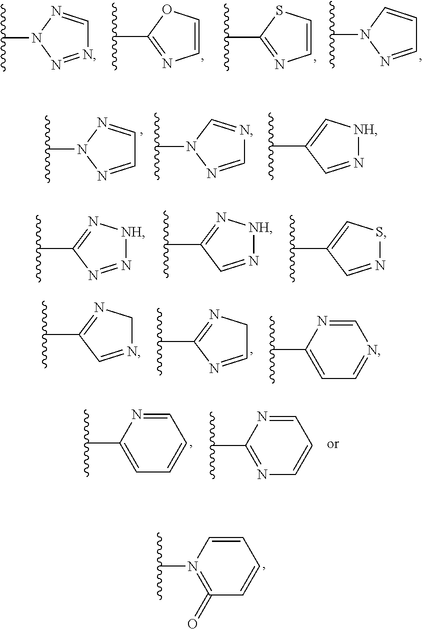

or a pharmaceutically acceptable solvate, pharmaceutically acceptable salt, and/or pharmaceutically acceptable prodrug thereof.

In some embodiments, the compound is selected from:

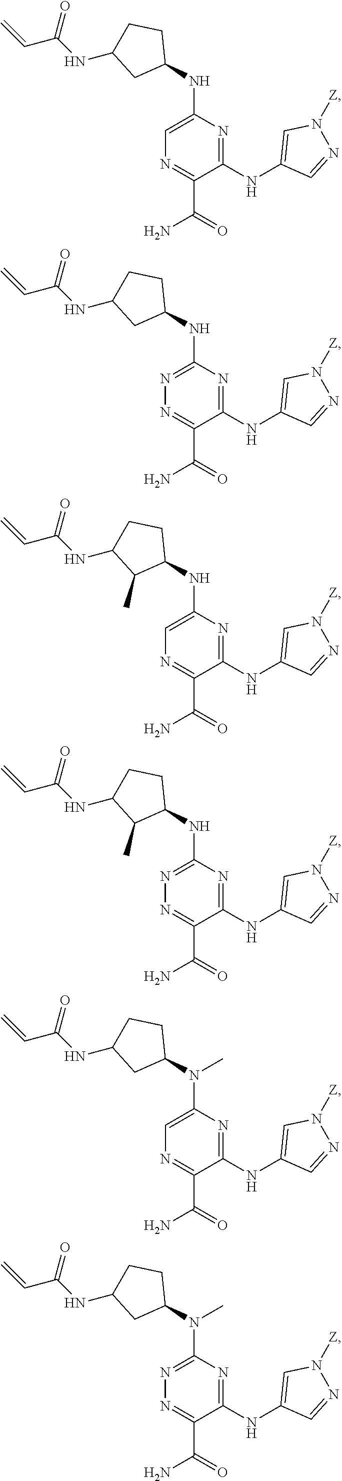

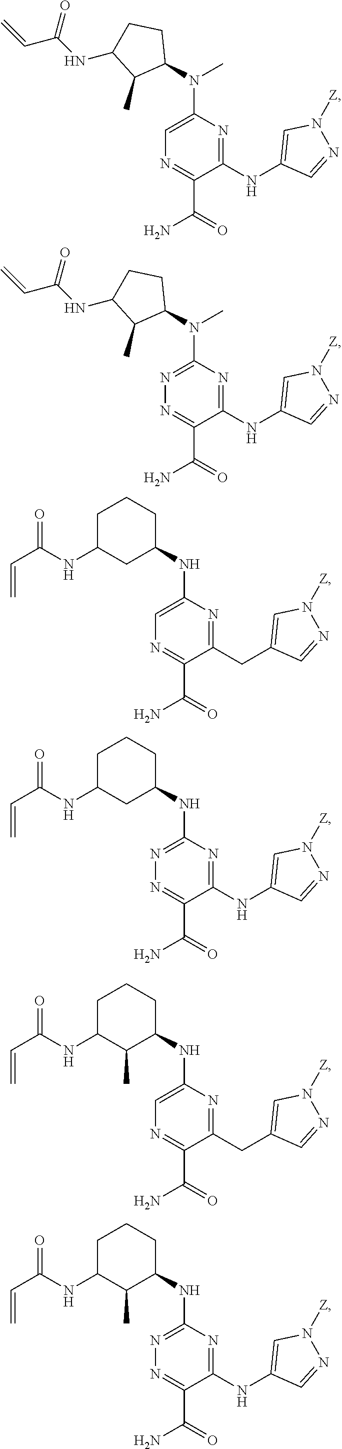

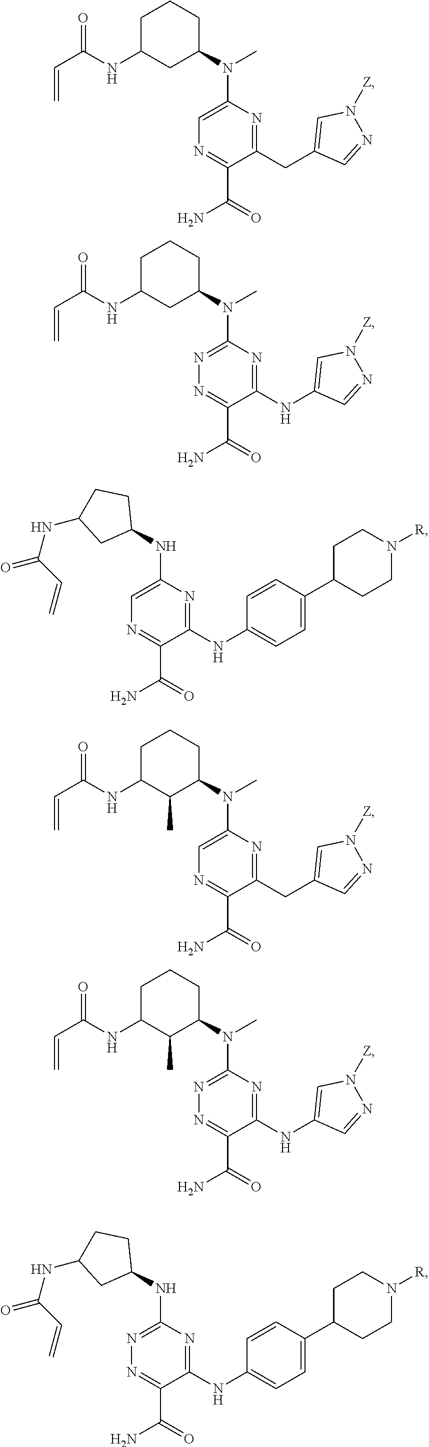

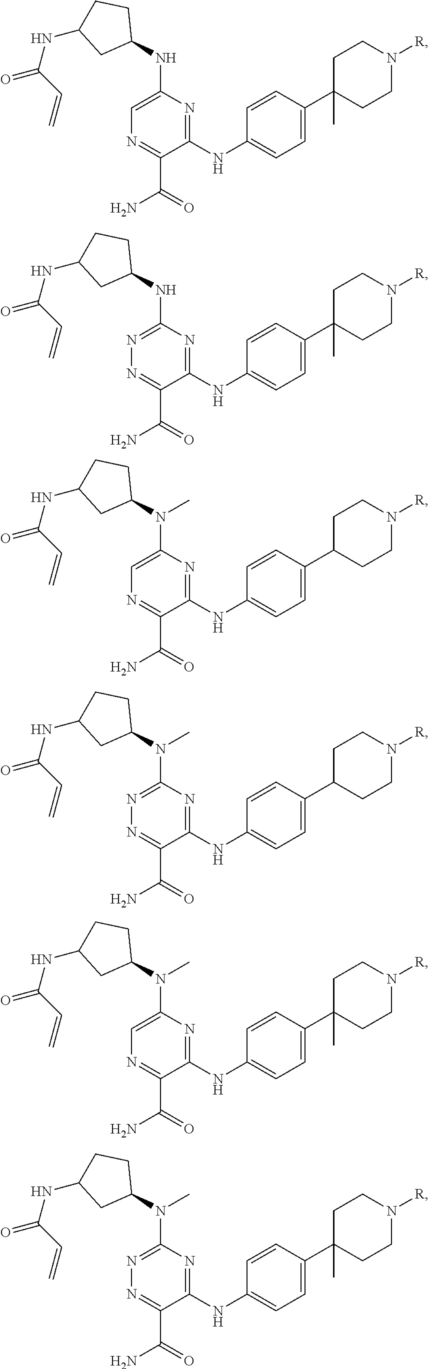

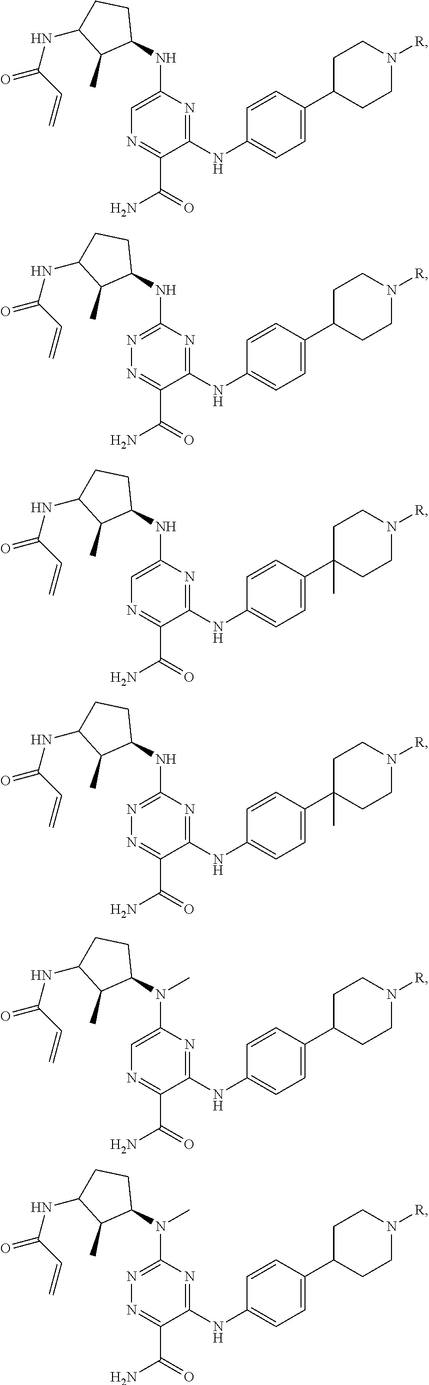

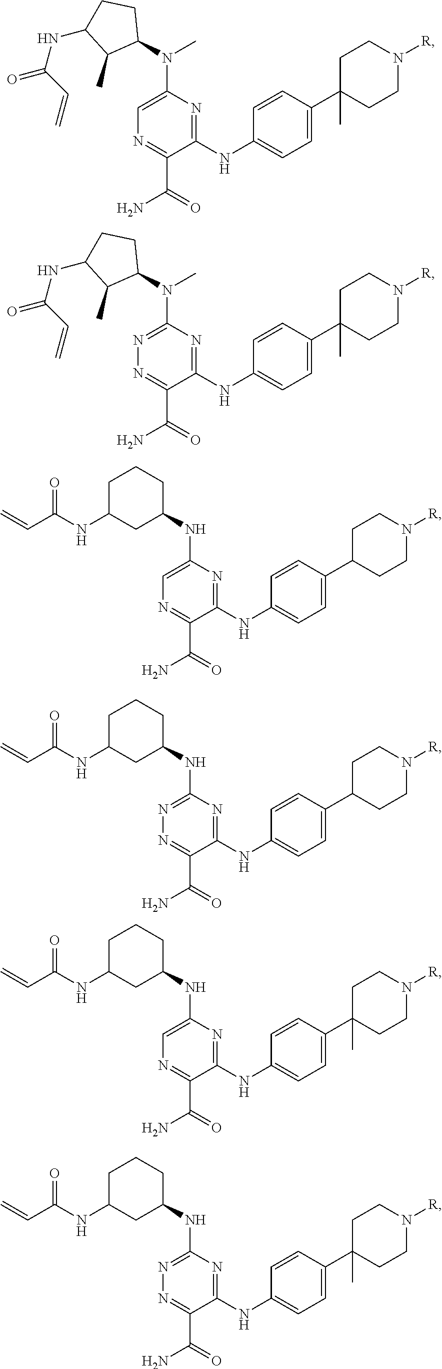

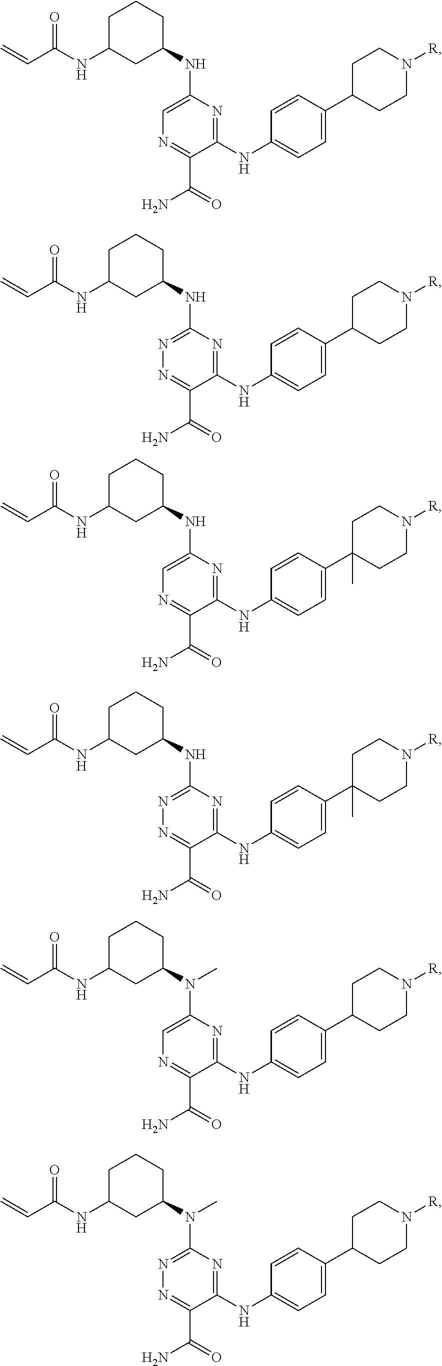

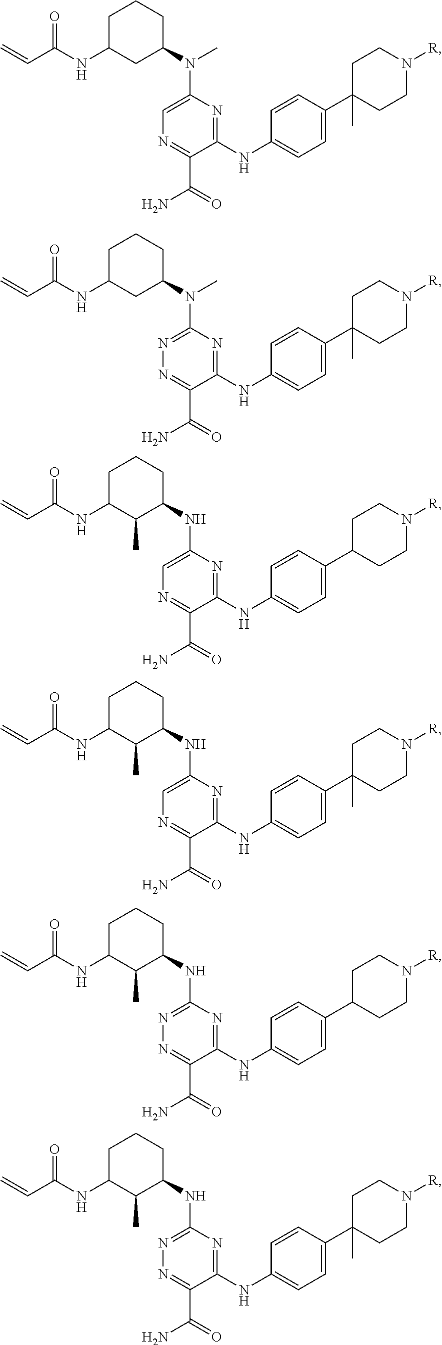

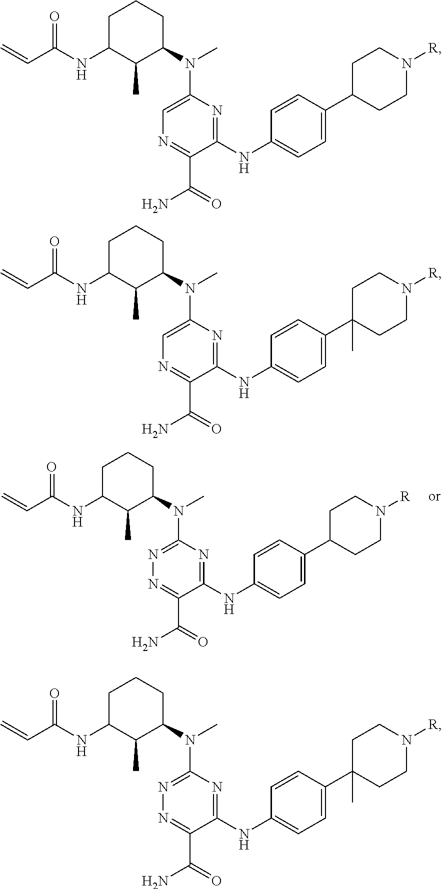

wherein Z and R are methyl, ethyl, isopropyl, cyclopropyl, cyclobutyl, or cyclopentyl, or a pharmaceutically acceptable solvate, pharmaceutically acceptable salt, and/or pharmaceutically acceptable prodrug thereof.

In some embodiments, the compound is selected from:

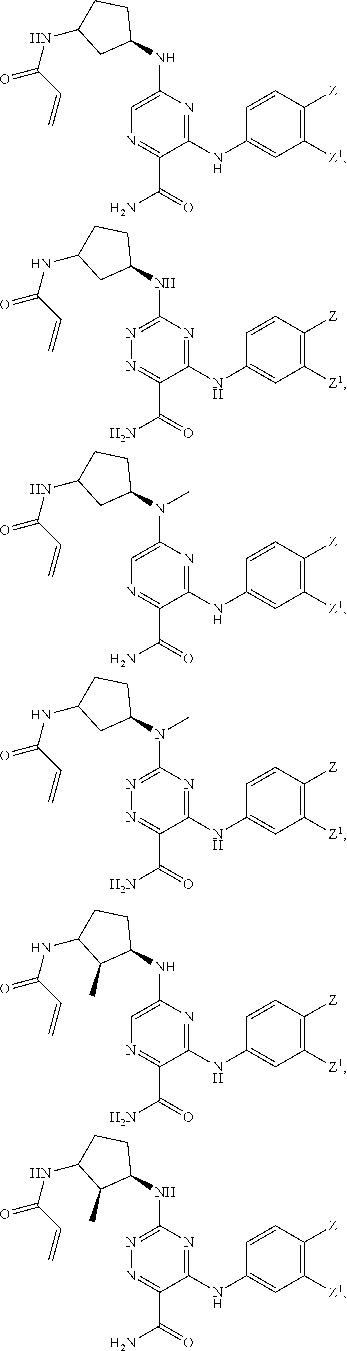

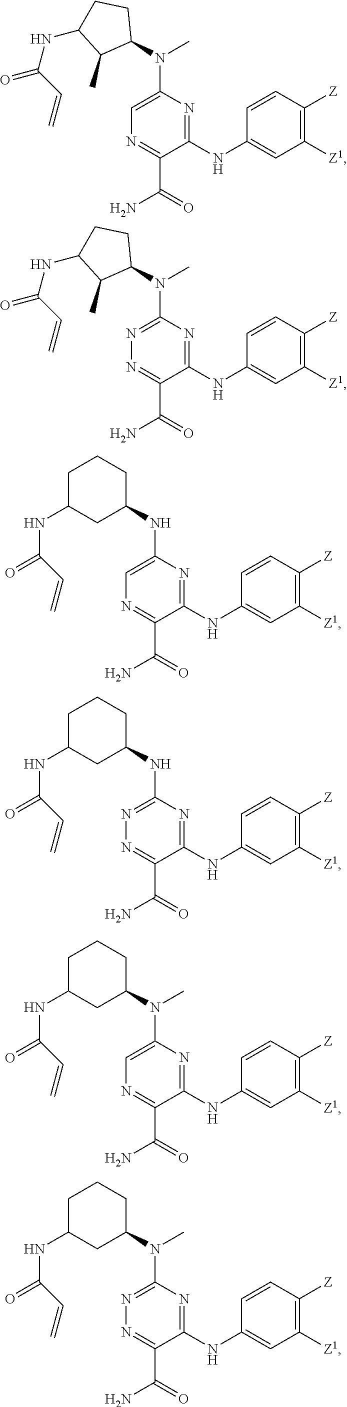

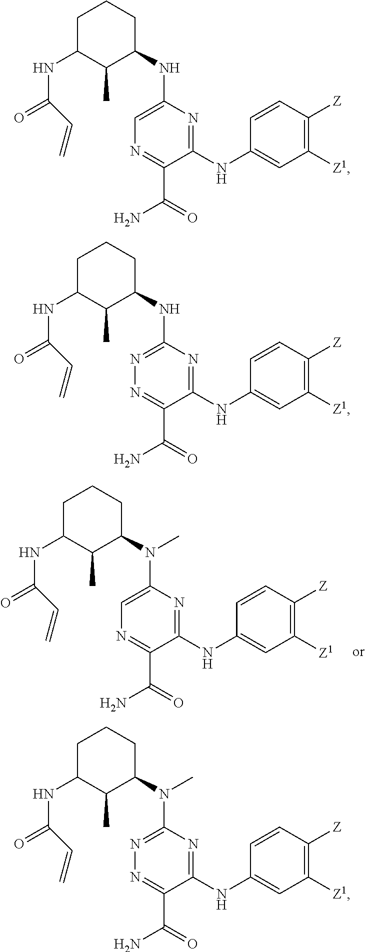

wherein Z and Z¹ are independently H, F, Cl, CN, methyl, ethyl, isopropyl, cyclopropyl, or CF₃, or a pharmaceutically acceptable solvate, pharmaceutically acceptable salt, and/or pharmaceutically acceptable prodrug thereof. In some embodiments, Z and Z¹ are independently F, Cl, CN, methyl, ethyl, isopropyl, cyclopropyl, or CF₃. In some embodiments, at least one of Z and Z¹ is other than H.

In some embodiments, the compound is selected from:

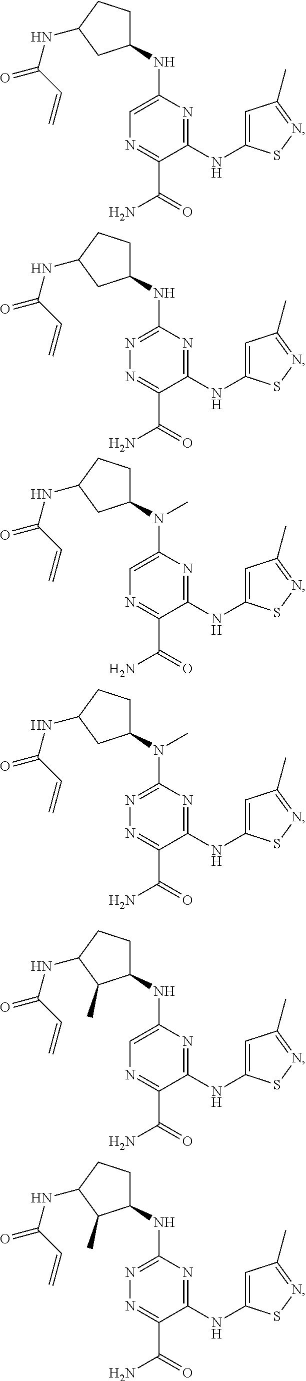

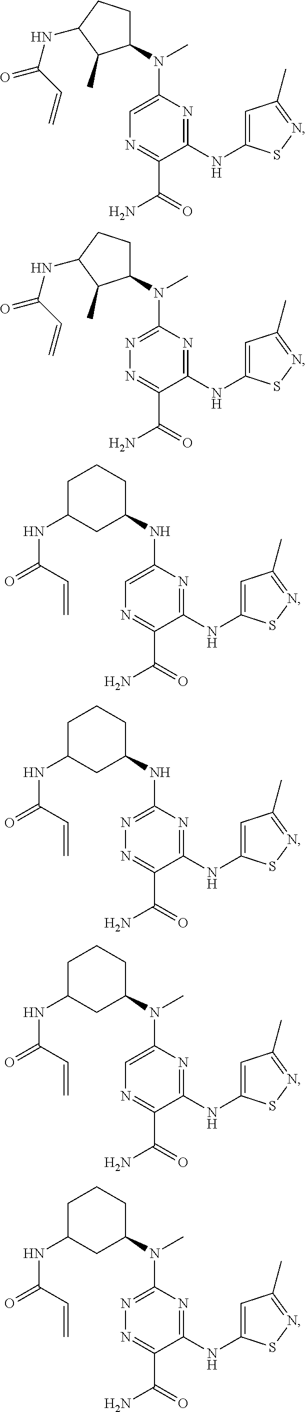

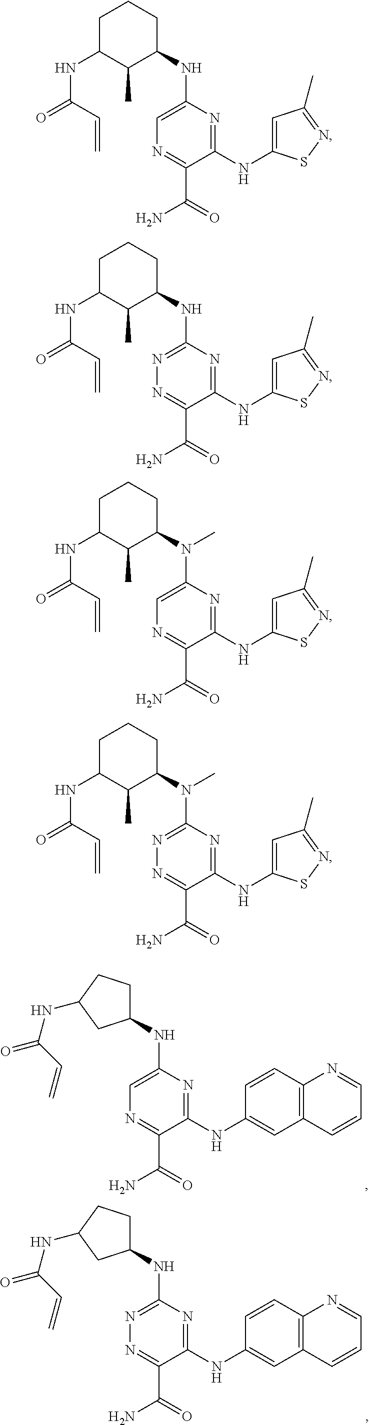

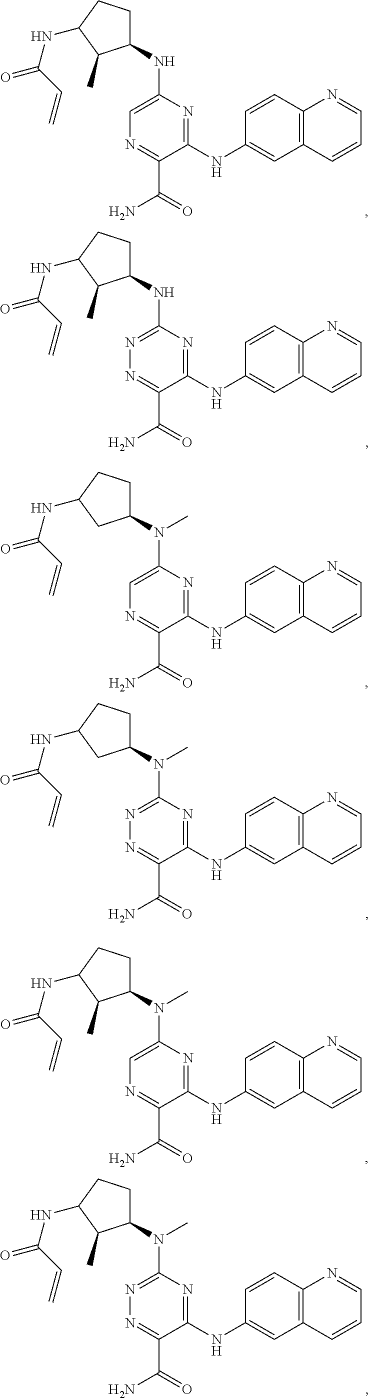

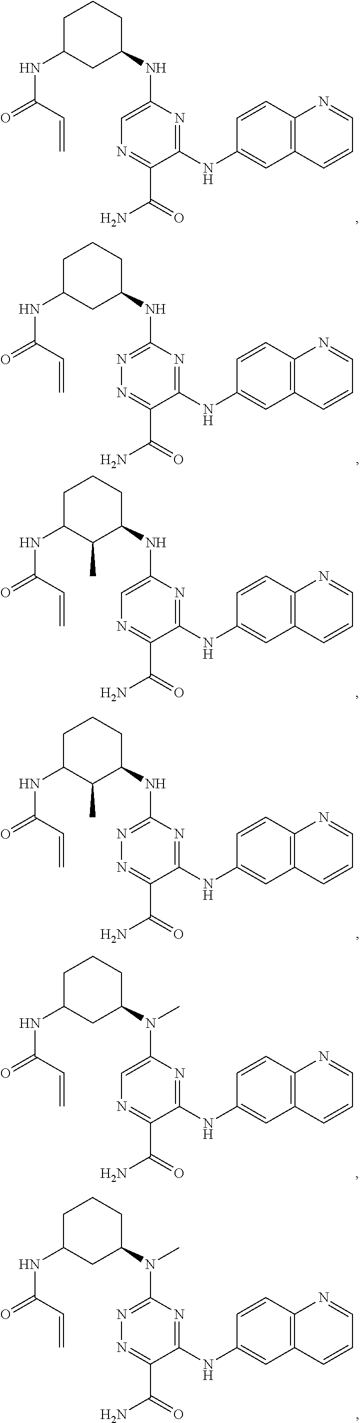

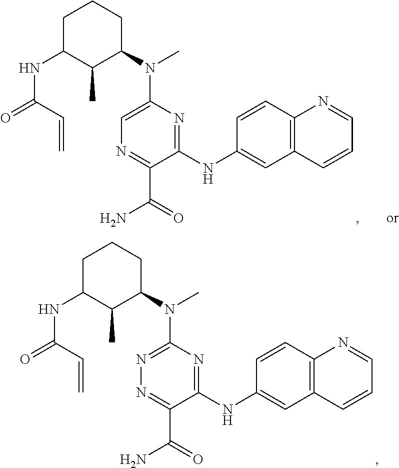

or a pharmaceutically acceptable solvate, pharmaceutically acceptable salt, and/or pharmaceutically acceptable prodrug thereof.

### Formula A-I Compounds

In particular embodiments, the compound is selected from the group consisting of compounds listed in Table N1:

TABLE N1
Formula A-I Compounds

| Compound Structure | Compound Name |
|---|---|
| 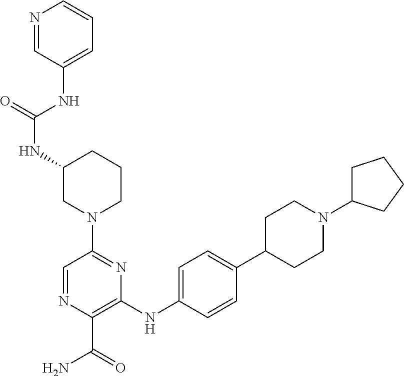 [SMILES: NC(=O)c1ncc(N2CCC[C@@H](NC(=O)Nc3cccnc3)C2)nc1Nc1ccc(C2CCN(C3CCCC3)CC2)cc1] | (R)-3-(4-(1-cyclopentylpiperidin-4- yl)phenylamino)-5-(3-(3-pyridin-3- ylureido)piperidin-1-yl)pyrazine-2- carboxamide |
| 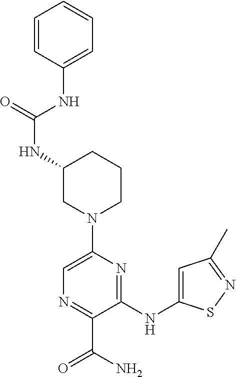 [SMILES: Cc1cc(Nc2nc(N3CCC[C@@H](NC(=O)Nc4ccccc4)C3)cnc2C(N)=O)sn1] | (R)-3-(3-methylisothiazol-5-ylamino)-5- (3-(3-phenylureido)piperidin-1- yl)pyrazine-2-carboxamide |
| 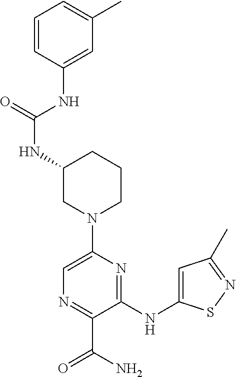 [SMILES: Cc1cccc(NC(=O)N[C@@H]2CCCN(c3cnc(C(N)=O)c(Nc4cc(C)ns4)n3)C2)c1] | (R)-3-(3-methylisothiazol-5-ylamino)-5- (3-(3-m-tolylureido)piperidin-1- yl)pyrazine-2-carboxamide |
| 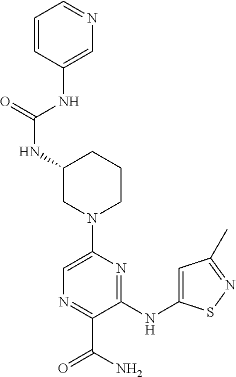 [SMILES: Cc1cc(Nc2nc(N3CCC[C@@H](NC(=O)Nc4cccnc4)C3)cnc2C(N)=O)sn1] | (R)-3-(3-methylisothiazol-5-ylamino)-5- (3-(3-pyridin-3-ylureido)piperidin-1- yl)pyrazine-2-carboxamide |
| 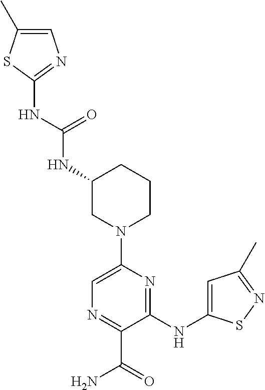 [SMILES: Cc1cc(Nc2nc(N3CCC[C@@H](NC(=O)Nc4ncc(C)s4)C3)cnc2C(N)=O)sn1] | (R)-3-(3-methylisothiazol-5-ylamino)-5- (3-(3-(5-methylthiazol-2- yl)ureido)piperidin-1-yl)pyrazine-2- carboxamide |
| 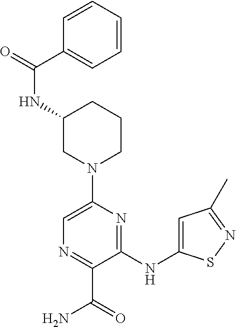 [SMILES: Cc1cc(Nc2nc(N3CCC[C@@H](NC(=O)c4ccccc4)C3)cnc2C(N)=O)sn1] | (R)-5-(3-benzamidopiperidin-1-yl)-3-(3- methylisothiazol-5-ylamino)pyrazine-2- carboxamide |
| 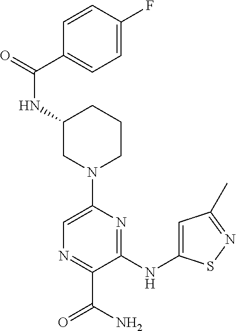 [SMILES: Cc1cc(Nc2nc(N3CCC[C@@H](NC(=O)c4ccc(F)cc4)C3)cnc2C(N)=O)sn1] | (R)-5-(3-(4-fluorobenzamido)piperidin-1- yl)-3-(3-methylisothiazol-5- ylamino)pyrazine-2-carboxamide |
| 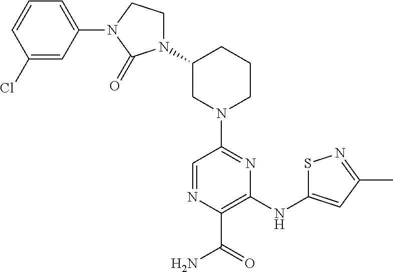 [SMILES: Cc1cc(Nc2nc(N3CCC[C@@H](N4CCN(c5cccc(Cl)c5)C4=O)C3)cnc2C(N)=O)sn1] | (R)-5-(3-(3-(3-chlorophenyl)-2- oxoimidazolidin-1-yl)piperidin-1-yl)-3- (3-methylisothiazol-5-ylamino)pyrazine- 2-carboxamide |
| 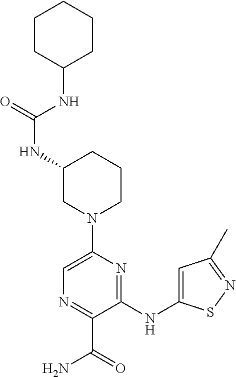 [SMILES: Cc1cc(Nc2nc(N3CCC[C@@H](NC(=O)NC4CCCCC4)C3)cnc2C(N)=O)sn1] | (R)-5-(3-(3-cyclohexylureido)piperidin- 1-yl)-3-(3-methylisothiazol-5- ylamino)pyrazine-2-carboxamide |
| 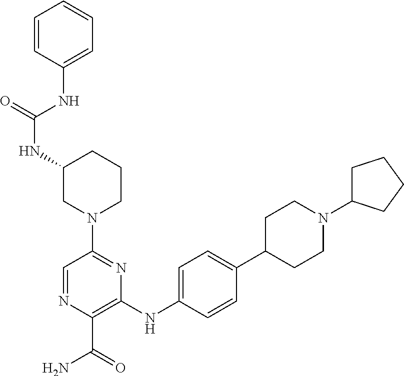 [SMILES: NC(=O)c1ncc(N2CCC[C@@H](NC(=O)Nc3ccccc3)C2)nc1Nc1ccc(C2CCN(C3CCCC3)CC2)cc1] | (R)-3-(4-(1-cyclopentylpiperidin-4- yl)phenylamino)-5-(3-(3- phenylureido)piperidin-1-yl)pyrazine-2- carboxamide |
| 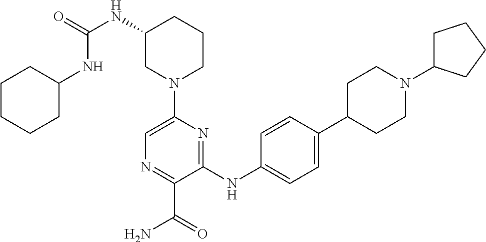 [SMILES: NC(=O)c1ncc(N2CCC[C@@H](NC(=O)NC3CCCCC3)C2)nc1Nc1ccc(C2CCN(C3CCCC3)CC2)cc1] | (R)-5-(3-(3-cyclohexylureido)piperidin- 1-yl)-3-(4-(1-cyclopentylpiperidin-4- yl)phenylamino)pyrazine-2-carboxamide |
| 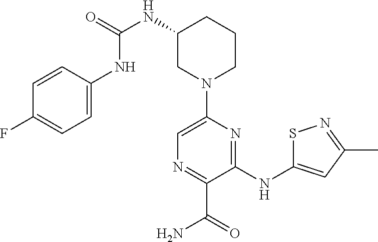 [SMILES: Cc1cc(Nc2nc(N3CCC[C@@H](NC(=O)Nc4ccc(F)cc4)C3)cnc2C(N)=O)sn1] | (R)-5-(3-(3-(4- fluorophenyl)ureido)piperidin-1-yl)-3-(3- methylisothiazol-5-ylamino)pyrazine-2- carboxamide |
| 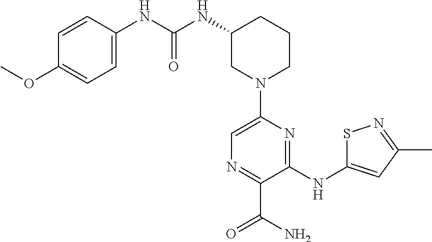 | (R)-5-(3-(3-(4- methoxyphenyl)ureido)piperidin-1-yl)-3- (3-methylisothiazol-5-ylamino)pyrazine- 2-carboxamide |
| 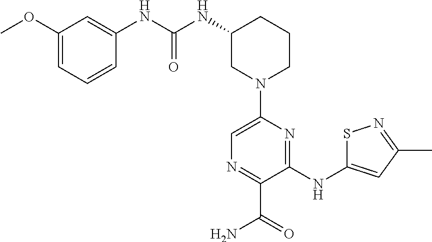 | (R)-5-(3-(3-(3- methoxyphenyl)ureido)piperidin-1-yl)-3- (3-methylisothiazol-5-ylamino)pyrazine- 2-carboxamide |
| 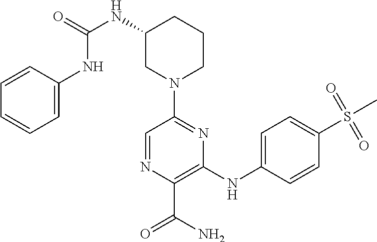 [SMILES: CS(=O)(=O)c1ccc(Nc2nc(N3CCC[C@@H](NC(=O)Nc4ccccc4)C3)cnc2C(N)=O)cc1] | (R)-3-(4-(methylsulfonyl)phenylamino)- 5-(3-(3-phenylureido)piperidin-1- yl)pyrazine-2-carboxamide |
| 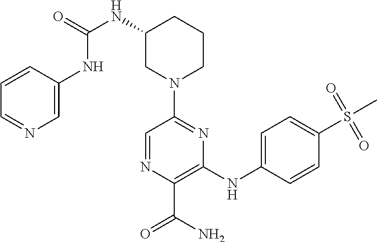 [SMILES: CS(=O)(=O)c1ccc(Nc2nc(N3CCC[C@@H](NC(=O)Nc4cccnc4)C3)cnc2C(N)=O)cc1] | (R)-3-(4-(methylsulfonyl)phenylamino)- 5-(3-(3-pyridin-3-ylureido)piperidin-1- yl)pyrazine-2-carboxamide |
| 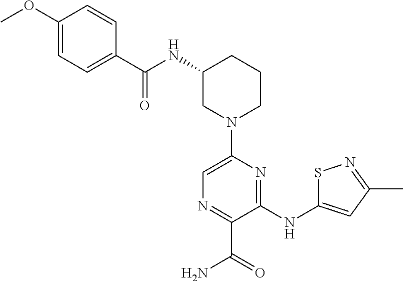 | (R)-5-(3-(4- methoxybenzamido)piperidin-1-yl)-3-(3- methylisothiazol-5-ylamino)pyrazine-2- carboxamide |
| 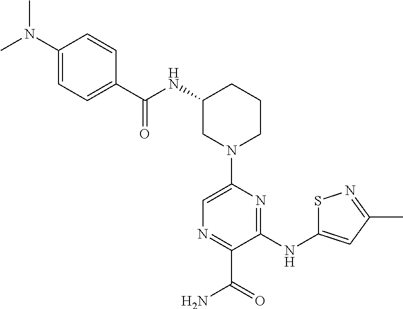 | (R)-5-(3-(4- (dimethylamino)benzamido)piperidin-1- yl)-3-(3-methylisothiazol-5- ylamino)pyrazine-2-carboxamide |
| 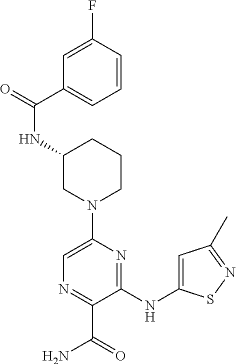 [SMILES: Cc1cc(Nc2nc(N3CCC[C@@H](NC(=O)c4cccc(F)c4)C3)cnc2C(N)=O)sn1] | (R)-5-(3-(3-fluorobenzamido)piperidin-1- yl)-3-(3-methylisothiazol-5- ylamino)pyrazine-2-carboxamide |
| 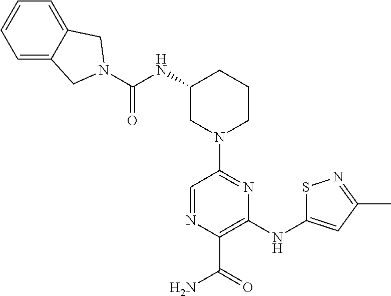 [SMILES: Cc1cc(Nc2nc(N3CCC[C@@H](NC(=O)N4Cc5ccccc5C4)C3)cnc2C(N)=O)sn1] | (R)-N-(1-(5-carbamoyl-6-(3- methylisothiazol-5-ylamino)pyrazin-2- yl)piperidin-3-yl)isoindoline-2- carboxamide |
| 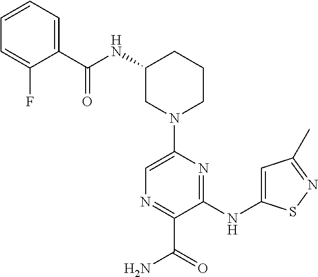 [SMILES: Cc1cc(Nc2nc(N3CCC[C@@H](NC(=O)c4ccccc4F)C3)cnc2C(N)=O)sn1] | (R)-5-(3-(2-fluorobenzamido)piperidin-1- yl)-3-(3-methylisothiazol-5- ylamino)pyrazine-2-carboxamide |
| 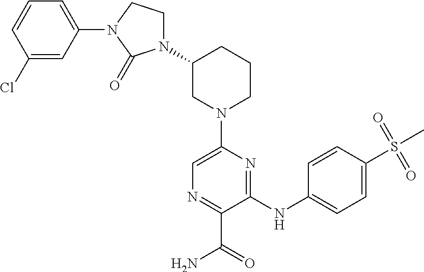 [SMILES: CS(=O)(=O)c1ccc(Nc2nc(N3CCC[C@@H](N4CCN(c5cccc(Cl)c5)C4=O)C3)cnc2C(N)=O)cc1] | (R)-5-(3-(3-(3-chlorophenyl)-2- oxoimidazolidin-1-yl)piperidin-1-yl)-3- (4- (methylsulfonyl)phenylamino)pyrazine- 2-carboxamide |
| 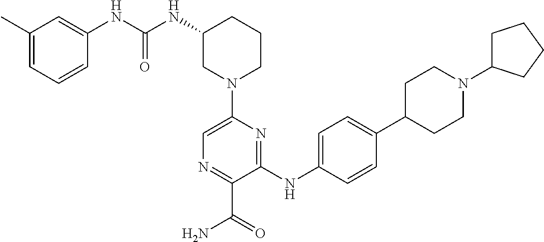 [SMILES: Cc1cccc(NC(=O)N[C@@H]2CCCN(c3cnc(C(N)=O)c(Nc4ccc(C5CCN(C6CCCC6)CC5)cc4)n3)C2)c1] | (R)-3-(4-(1-cyclopentylpiperidin-4- yl)phenylamino)-5-(3-(3-m- tolylureido)piperidin-1-yl)pyrazine-2- carboxamide |
| 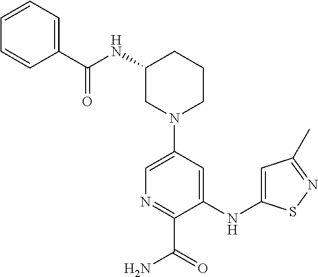 [SMILES: Cc1cc(Nc2cc(N3CCC[C@@H](NC(=O)c4ccccc4)C3)cnc2C(N)=O)sn1] | (R)-5-(3-benzamidopiperidin-1-yl)-3-(3- methylisothiazol-5-ylamino)picolinamide |
| 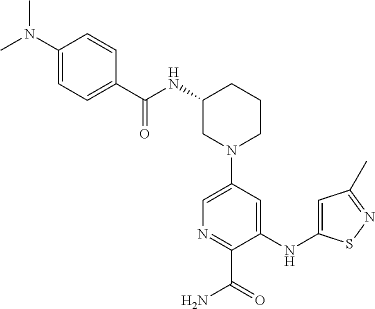 [SMILES: Cc1cc(Nc2cc(N3CCC[C@@H](NC(=O)c4ccc(N(C)C)cc4)C3)cnc2C(N)=O)sn1] | (R)-5-(3-(4- (dimethylamino)benzamido)piperidin-1- yl)-3-(3-methylisothiazol-5- ylamino)picolinamide |
| 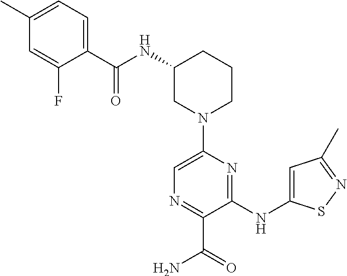 [SMILES: Cc1ccc(C(=O)N[C@@H]2CCCN(c3cnc(C(N)=O)c(Nc4cc(C)ns4)n3)C2)c(F)c1] | (R)-5-(3-(2-fluoro-4- methylbenzamido)piperidin-1-yl)-3-(3- methylisothiazol-5-ylamino)pyrazine-2- carboxamide |
| 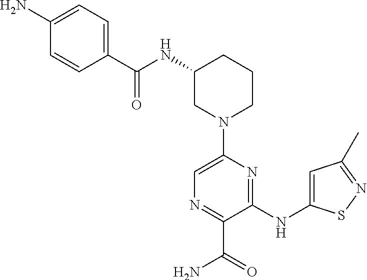 [SMILES: Cc1cc(Nc2nc(N3CCC[C@@H](NC(=O)c4ccc(N)cc4)C3)cnc2C(N)=O)sn1] | (R)-5-(3-(4-aminobenzamido)piperidin- 1-yl)-3-(3-methylisothiazol-5- ylamino)pyrazine-2-carboxamide |
| 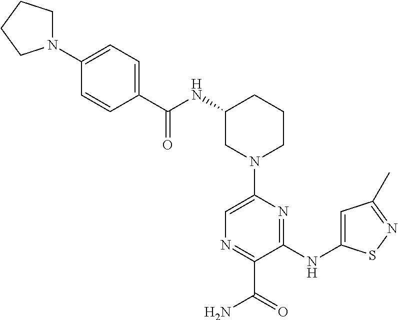 [SMILES: Cc1cc(Nc2nc(N3CCC[C@@H](NC(=O)c4ccc(N5CCCC5)cc4)C3)cnc2C(N)=O)sn1] | (R)-3-(3-methylisothiazol-5-ylamino)-5- (3-(4-(pyrrolidin-1- yl)benzamido)piperidin-1-yl)pyrazine-2- carboxamide |
| 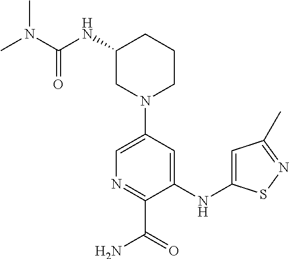 [SMILES: Cc1cc(Nc2cc(N3CCC[C@@H](NC(=O)N(C)C)C3)cnc2C(N)=O)sn1] | (R)-5-(3-(3,3-dimethylureido)piperidin-1- yl)-3-(3-methylisothiazol-5- ylamino)picolinamide |
| 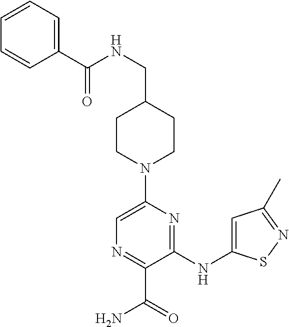 [SMILES: Cc1cc(Nc2nc(N3CCC(CNC(=O)c4ccccc4)CC3)cnc2C(N)=O)sn1] | 5-(4-(benzamidomethyl)piperidin-1-yl)- 3-(3-methylisothiazol-5- ylamino)pyrazine-2-carboxamide |
| 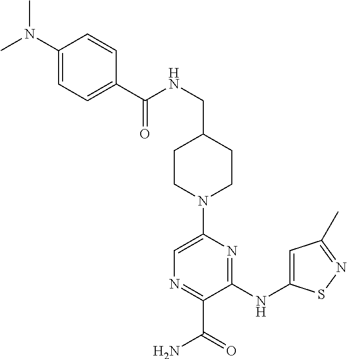 [SMILES: Cc1cc(Nc2nc(N3CCC(CNC(=O)c4ccc(N(C)C)cc4)CC3)cnc2C(N)=O)sn1] | 5-(4-((4-(dimethyl- amino)benzamido)methyl)piperidin- 1-yl)-3-(3-methylisothiazol-5- ylamino)pyrazine-2-carboxamide |
| 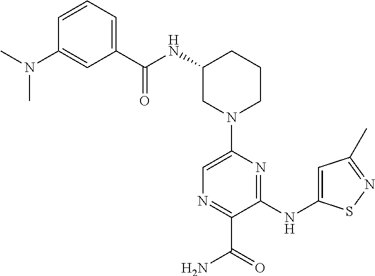 [SMILES: Cc1cc(Nc2nc(N3CCC[C@@H](NC(=O)c4cccc(N(C)C)c4)C3)cnc2C(N)=O)sn1] | (R)-5-(3-(3- (dimethylamino)benzamido)piperidin-1- yl)-3-(3-methylisothiazol-5- ylamino)pyrazine-2-carboxamide |
| 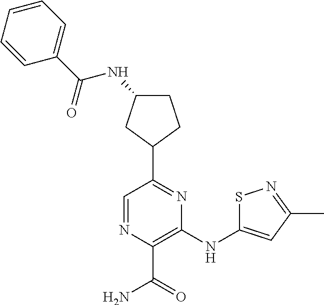 [SMILES: Cc1cc(Nc2nc(C3CC[C@@H](NC(=O)c4ccccc4)C3)cnc2C(N)=O)sn1] | (R)-5-(3-benzamidopyrrolidin-1-yl)-3-(3- methylisothiazol-5-ylamino)pyrazine-2- carboxamide |
| 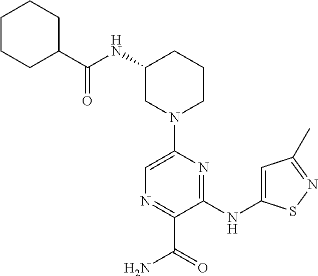 [SMILES: Cc1cc(Nc2nc(N3CCC[C@@H](NC(=O)C4CCCCC4)C3)cnc2C(N)=O)sn1] | (R)-5-(3- (cyclohexanecarboxamido)piperidin-1- yl)-3-(3-methylisothiazol-5- ylamino)pyrazine-2-carboxamide |
| 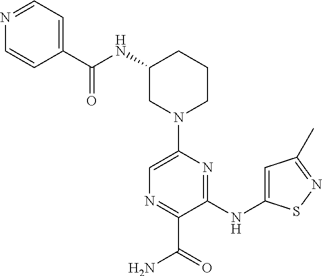 [SMILES: Cc1cc(Nc2nc(N3CCC[C@@H](NC(=O)c4ccncc4)C3)cnc2C(N)=O)sn1] | (R)-5-(3-(isonicotinamido)piperidin-1- yl)-3-(3-methylisothiazol-5- ylamino)pyrazine-2-carboxamide |
| 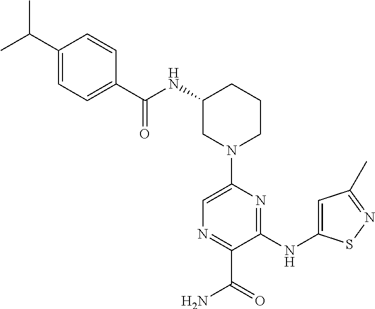 [SMILES: Cc1cc(Nc2nc(N3CCC[C@@H](NC(=O)c4ccc(C(C)C)cc4)C3)cnc2C(N)=O)sn1] | (R)-5-(3-(4- isopropylbenzamido)piperidin-1-yl)-3-(3- methylisothiazol-5-ylamino)pyrazine-2- carboxamide |
| 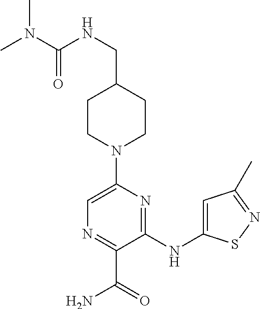 [SMILES: Cc1cc(Nc2nc(N3CCC(CNC(=O)N(C)C)CC3)cnc2C(N)=O)sn1] | 5-(4-((3,3- dimethylureido)methyl)piperidin-1-yl)-3- (3-methylisothiazol-5-ylamino)pyrazine- 2-carboxamide |
| 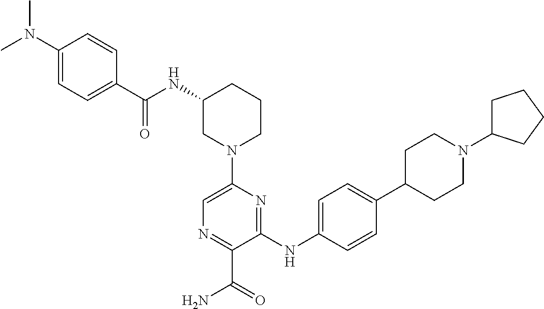 [SMILES: CN(C)c1ccc(C(=O)N[C@@H]2CCCN(c3cnc(C(N)=O)c(Nc4ccc(C5CCN(C6CCCC6)CC5)cc4)n3)C2)cc1] | (R)-3-(4-(1-cyclopentylpiperidin-4- yl)phenylamino)-5-(3-(4- (dimethylamino)benzamido)piperidin-1- yl)pyrazine-2-carboxamide |
| 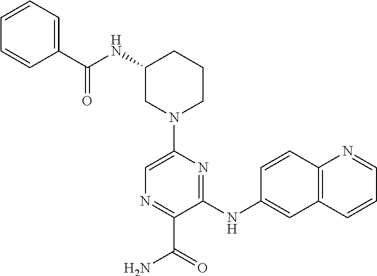 [SMILES: NC(=O)c1ncc(N2CCC[C@@H](NC(=O)c3ccccc3)C2)nc1Nc1ccc2ncccc2c1] | (R)-5-(3-benzamidopiperidin-1-yl)-3- (quinolin-6-ylamino)pyrazine-2- carboxamide |
| 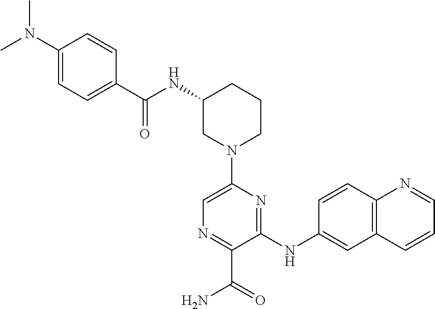 [SMILES: CN(C)c1ccc(C(=O)N[C@@H]2CCCN(c3cnc(C(N)=O)c(Nc4ccc5ncccc5c4)n3)C2)cc1] | (R)-5-(3-(4- (dimethylamino)benzamido)piperidin-1- yl)-3-(quinolin-6-ylamino)pyrazine-2- carboxamide |
| 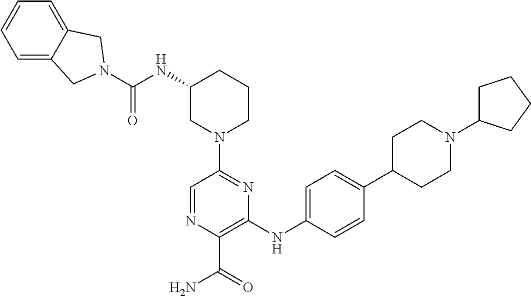 [SMILES: NC(=O)c1ncc(N2CCC[C@@H](NC(=O)N3Cc4ccccc4C3)C2)nc1Nc1ccc(C2CCN(C3CCCC3)CC2)cc1] | (R)-N-(1-(5-carbamoyl-6-(4-(1- cyclopentylpiperidin-4- yl)phenylamino)pyrazin-2-yl)piperidin-3- yl)isoindoline-2-carboxamide |
| 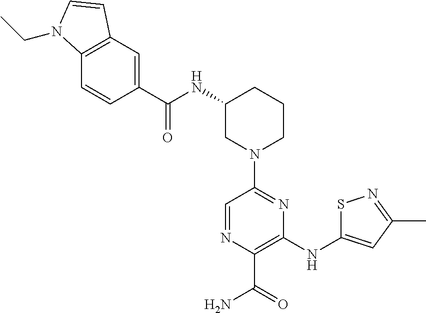 [SMILES: CCn1ccc2cc(C(=O)N[C@@H]3CCCN(c4cnc(C(N)=O)c(Nc5cc(C)ns5)n4)C3)ccc21] | (R)-N-(1-(5-carbamoyl-6-(3- methylisothiazol-5-ylamino)pyrazin-2- yl)piperidin-3-yl)-1-ethyl-1H-indole-5- carboxamide |
| 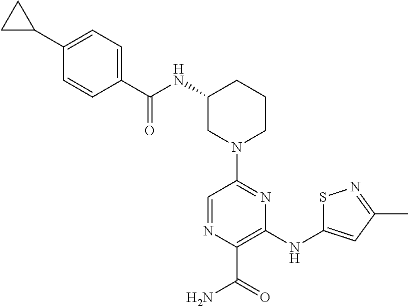 [SMILES: Cc1cc(Nc2nc(N3CCC[C@@H](NC(=O)c4ccc(C5CC5)cc4)C3)cnc2C(N)=O)sn1] | (R)-5-(3-(4- cyclopropylbenzamido)piperidin-1-yl)-3- (3-methylisothiazol-5-ylamino)pyrazine- 2-carboxamide |
| 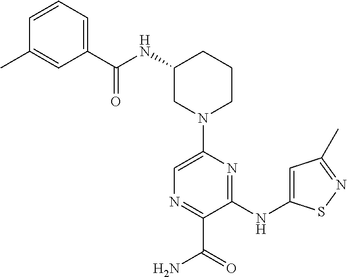 [SMILES: Cc1cccc(C(=O)N[C@@H]2CCCN(c3cnc(C(N)=O)c(Nc4cc(C)ns4)n3)C2)c1] | (R)-5-(3-(3-methylbenzamido)piperidin- 1-yl)-3-(3-methylisothiaozl-5- ylamino)pyrazine-2-carboxamide |
| 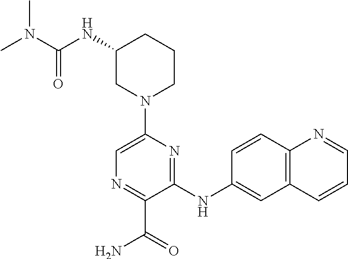 [SMILES: CN(C)C(=O)N[C@@H]1CCCN(c2cnc(C(N)=O)c(Nc3ccc4ncccc4c3)n2)C1] | (R)-5-(3-(3,3-dimethylureido)piperidin-1- yl)-3-(quinolin-6-ylamino)pyrazine-2- carboxamide |
| 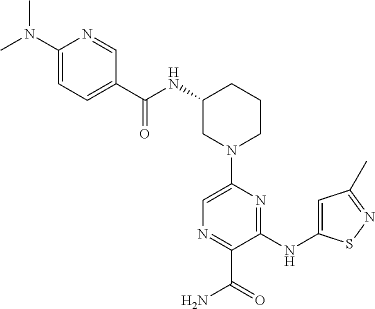 [SMILES: Cc1cc(Nc2nc(N3CCC[C@@H](NC(=O)c4ccc(N(C)C)nc4)C3)cnc2C(N)=O)sn1] | (R)-5-(3-(6- (dimethylamino)nicotinamido)piperidin- 1-yl)-3-(3-methylisothiazol-5- ylamino)pyrazine-2-carboxamide |
| 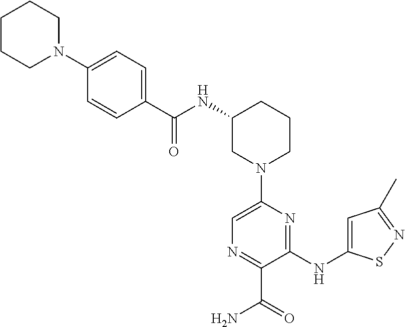 [SMILES: Cc1cc(Nc2nc(N3CCC[C@@H](NC(=O)c4ccc(N5CCCCC5)cc4)C3)cnc2C(N)=O)sn1] | (R)-3-(3-methylisothiazol-5-ylamino)-5- (3-(4-(piperidin-1- yl)benzamido)piperidin-1-yl)pyrazine-2- carboxamide |
| 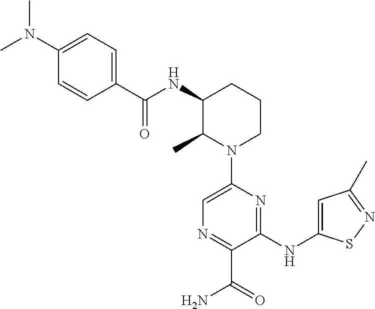 [SMILES: Cc1cc(Nc2nc(N3CCC[C@H](NC(=O)c4ccc(N(C)C)cc4)[C@@H]3C)cnc2C(N)=O)sn1] | 5-((2S,3S)-3-(4- (dimethylamino)benzamido)-2- methylpiperidin-1-yl)-3-(3- methylisothiazol-5-ylamino)pyrazine-2- carboxamide |
| 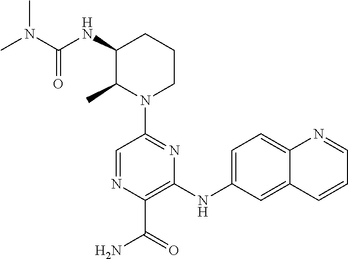 [SMILES: C[C@H]1[C@@H](NC(=O)N(C)C)CCCN1c1cnc(C(N)=O)c(Nc2ccc3ncccc3c2)n1] | 5-((2S,3S)-3-(3,3-dimethylureido)-2- methylpiperidin-1-yl)-3-(quinolin-6- ylamino)pyrazine-2-carboxamide |
| 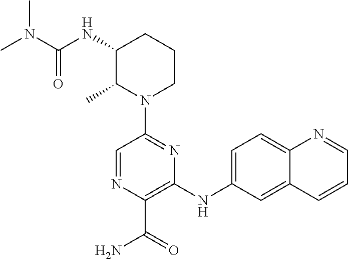 [SMILES: C[C@@H]1[C@H](NC(=O)N(C)C)CCCN1c1cnc(C(N)=O)c(Nc2ccc3ncccc3c2)n1] | 5-((2R,3R)-3-(3,3-dimethylureido)-2- methylpiperidin-1-yl)-3-(quinolin-6- ylamino)pyrazine-2-carboxamide |
| 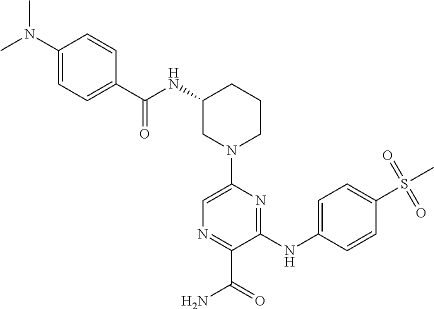 | (R)-5-(3-(4- (dimethylamino)benzamido)piperidin-1- yl)-3-(4- (methylsulfonyl)phenylamino)pyrazine- 2-carboxamide |
| 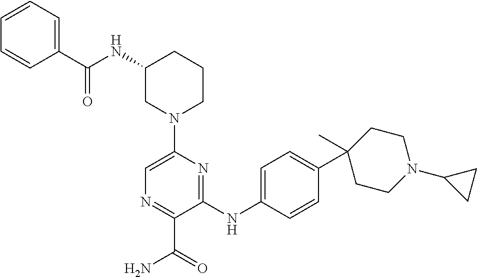 [SMILES: CC1(c2ccc(Nc3nc(N4CCC[C@@H](NC(=O)c5ccccc5)C4)cnc3C(N)=O)cc2)CCN(C2CC2)CC1] | (R)-5-(3-benzamidopiperidin-1-yl)-3-(4- (1-cyclopropyl-4-methylpiperidin-4- yl)phenylamino)pyrazine-2-carboxamide |
| 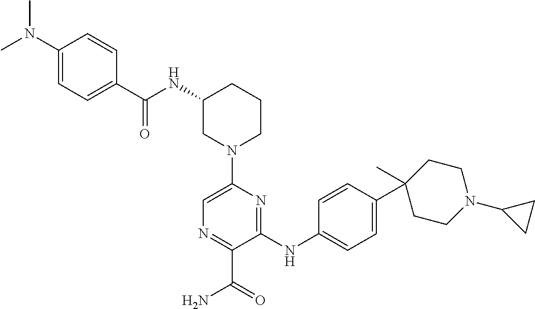 [SMILES: CN(C)c1ccc(C(=O)N[C@@H]2CCCN(c3cnc(C(N)=O)c(Nc4ccc(C5(C)CCN(C6CC6)CC5)cc4)n3)C2)cc1] | (R)-3-(4-(1-cyclopropyl-4- methylpiperidin-4-yl)phenylamino)-5-(3- (4-(dimethylamino)benzamido)piperidin- 1-yl)pyrazine-2-carboxamide |
| 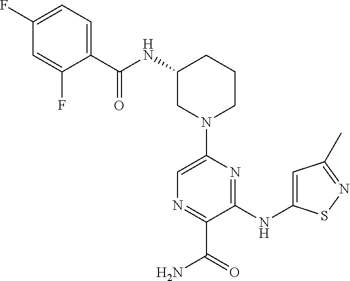 [SMILES: Cc1cc(Nc2nc(N3CCC[C@@H](NC(=O)c4ccc(F)cc4F)C3)cnc2C(N)=O)sn1] | (R)-5-(3-(2,4- difluorobenzamido)piperidin-1-yl)-3-(3- methylisothiazol-5-ylamino)pyrazine-2- carboxamide |
| 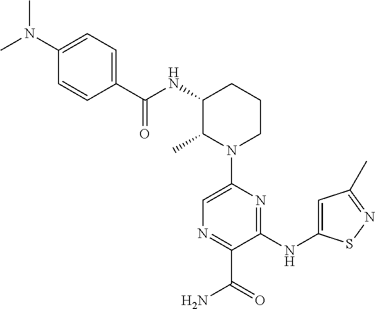 [SMILES: Cc1cc(Nc2nc(N3CCC[C@@H](NC(=O)c4ccc(N(C)C)cc4)[C@H]3C)cnc2C(N)=O)sn1] | 5-((2R,3R)-3-(4- (dimethylamino)benzamido)-2- methylpiperidin-1-yl)-3-(3- methylisothiazol-5-ylamino)pyrazine-2- carboxamide |
| 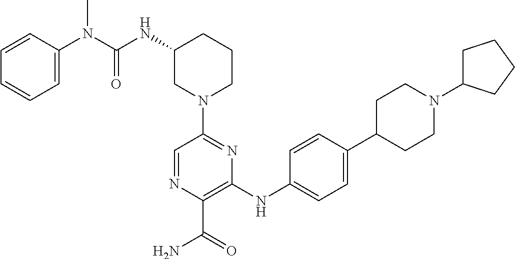 [SMILES: CN(C(=O)N[C@@H]1CCCN(c2cnc(C(N)=O)c(Nc3ccc(C4CCN(C5CCCC5)CC4)cc3)n2)C1)c1ccccc1] | (R)-3-(4-(1-cyclopentylpiperidin-4- yl)phenylamino)-5-(3-(3-methyl-3- phenylureido)piperidin-1-yl)pyrazine-2- carboxamide |
| 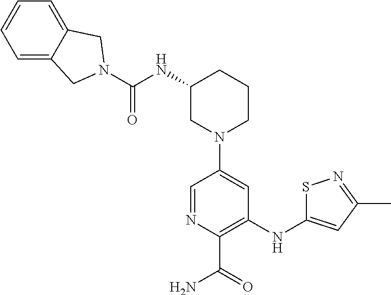 [SMILES: Cc1cc(Nc2cc(N3CCC[C@@H](NC(=O)N4Cc5ccccc5C4)C3)cnc2C(N)=O)sn1] | (R)-N-(1-(6-carbamoyl-5-(3- methylisothiazol-5-ylamino)pyridin-3- yl)piperidin-3-yl)isoindoline-2- carboxamide |
| 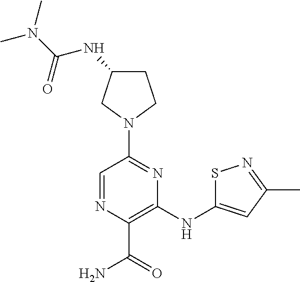 | (R)-5-(3-(3,3-dimethylureido)pyrrolidin- 1-yl)-3-(3-methylisothiazol-5- ylamino)pyrazine-2-carboxamide |
| 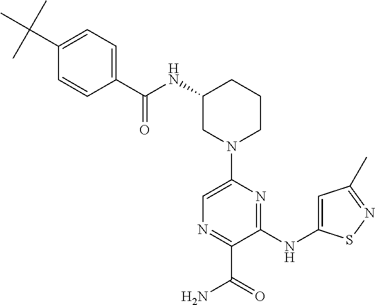 [SMILES: Cc1cc(Nc2nc(N3CCC[C@@H](NC(=O)c4ccc(C(C)(C)C)cc4)C3)cnc2C(N)=O)sn1] | (R)-5-(3-(4-tert- butylbenzamido)piperidin-1-yl)-3-(3- methylisothiazol-5-ylamino)pyrazine-2- carboxamide |
| 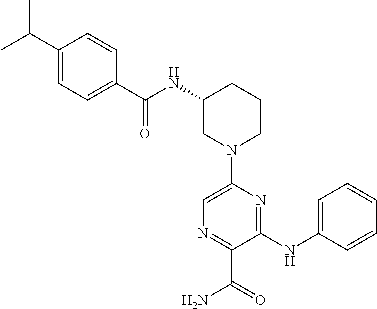 [SMILES: CC(C)c1ccc(C(=O)N[C@@H]2CCCN(c3cnc(C(N)=O)c(Nc4ccccc4)n3)C2)cc1] | (R)-5-(3-(4- isopropylbenzamido)piperidin-1-yl)-3- (phenylamino)pyrazine-2-carboxamide |
| 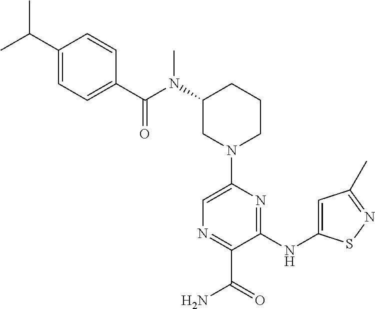 [SMILES: Cc1cc(Nc2nc(N3CCC[C@@H](N(C)C(=O)c4ccc(C(C)C)cc4)C3)cnc2C(N)=O)sn1] | (R)-5-(3-(4-isopropyl-N- methylbenzamido)piperidin-1-yl)-3-(3- methylisothiazol-5-ylamino)pyrazine-2- carboxamide |
| 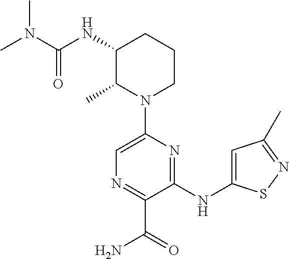 [SMILES: Cc1cc(Nc2nc(N3CCC[C@@H](NC(=O)N(C)C)[C@H]3C)cnc2C(N)=O)sn1] | 5-((2R,3R)-3-(3,3-dimethylureido)-2- methylpiperidin-1-yl)-3-(3- methylisothiazol-5-ylamino)pyrazine-2- carboxamide |
| 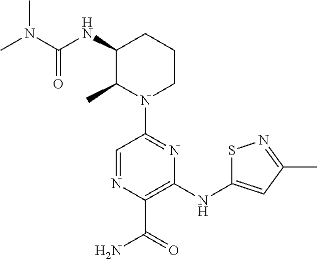 | 5-((2S,3S)-3-(3,3-dimethylureido)-2- methylpiperidin-1-yl)-3-(3- methylisothiazol-5-ylamino)pyrazine-2- carboxamide |
| 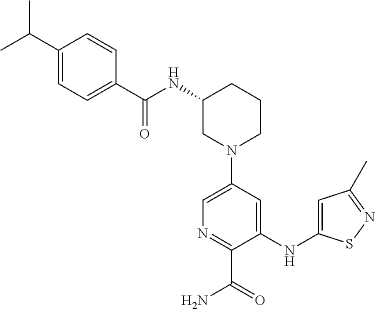 [SMILES: Cc1cc(Nc2cc(N3CCC[C@@H](NC(=O)c4ccc(C(C)C)cc4)C3)cnc2C(N)=O)sn1] | (R)-5-(3-(4- isopropylbenzamido)piperidin-1-yl)-3-(3- methylisothiazol-5-ylamino)picolinamide |
| 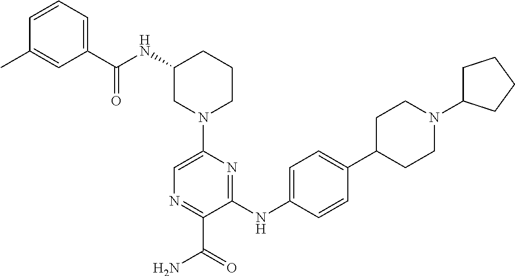 [SMILES: Cc1cccc(C(=O)N[C@@H]2CCCN(c3cnc(C(N)=O)c(Nc4ccc(C5CCN(C6CCCC6)CC5)cc4)n3)C2)c1] | (R)-3-(4-(1-cyclopentylpiperidin-4- yl)phenylamino)-5-(3-(3- methylbenzamido)piperidin-1- yl)pyrazine-2-carboxamide |
| 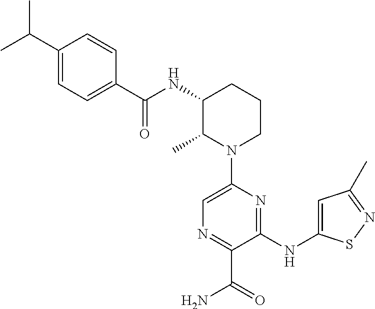 [SMILES: Cc1cc(Nc2nc(N3CCC[C@@H](NC(=O)c4ccc(C(C)C)cc4)[C@H]3C)cnc2C(N)=O)sn1] | 5-((2R,3R)-3-(4-isopropylbenzamido)-2- methylpiperidin-1-yl)-3-(3- methylisothiazol-5-ylamino)pyrazine-2- carboxamide |
| 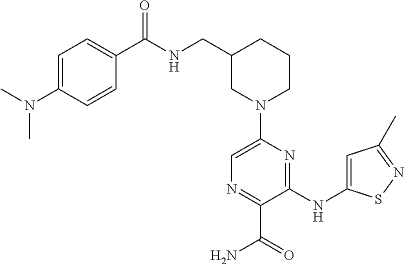 [SMILES: Cc1cc(Nc2nc(N3CCCC(CNC(=O)c4ccc(N(C)C)cc4)C3)cnc2C(N)=O)sn1] | 5-(3-((4-(dimethyl- amino)benzamido)methyl)piperidin- 1-yl)-3-(3-methylisothiazol-5- ylamino)pyrazine-2-carboxamide |
| 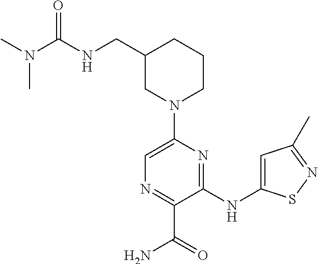 [SMILES: Cc1cc(Nc2nc(N3CCCC(CNC(=O)N(C)C)C3)cnc2C(N)=O)sn1] | 5-(3-((3,3- dimethylureido)methyl)piperidin-1-yl)-3- (3-methylisothiazol-5-ylamino)pyrazine- 2-carboxamide |
| 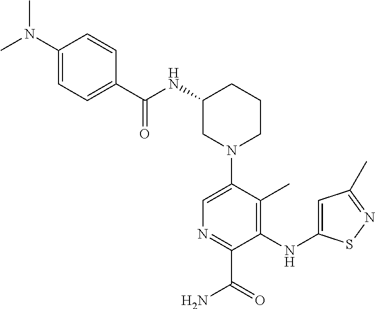 [SMILES: Cc1cc(Nc2c(C(N)=O)ncc(N3CCC[C@@H](NC(=O)c4ccc(N(C)C)cc4)C3)c2C)sn1] | (R)-5-(3-(4- (dimethylamino)benzamido)piperidin-1- yl)-4-methyl-3-(3-methylisothiazol-5- ylamino)picolinamide |
| 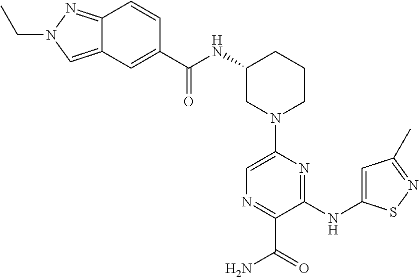 [SMILES: CCn1cc2cc(C(=O)N[C@@H]3CCCN(c4cnc(C(N)=O)c(Nc5cc(C)ns5)n4)C3)ccc2n1] | (R)-N-(1-(5-carbamoyl-6-(3- methylisothiazol-5-ylamino)pyrazin-2- yl)piperidin-3-yl)-2-ethyl-2H-indazole-5- carboxamide |
| 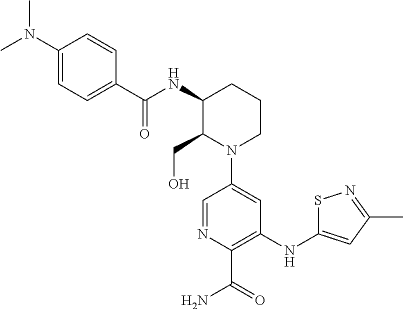 | 5-((2R,3S)-3-(4- (dimethylamino)benzamido)-2- (hydroxymethyl)piperidin-1-yl)-3-(3- methylisothiazol-5-ylamino)pyrazine-2- carboxamide |
| 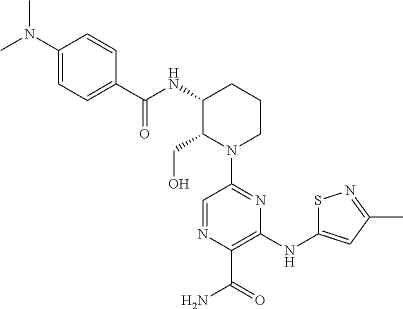 | 5-((2S,3R)-3-(4- (dimethylamino)benzamido)-2- (hydroxymethyl)piperidin-1-yl)-3-(3- methylisothiazol-5-ylamino)pyrazine-2- carboxamide |
| 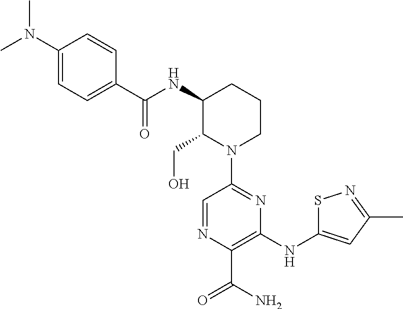 | 5-((2S,3S)-3-(4- (dimethylamino)benzamido)-2- (hydroxymethyl)piperidin-1-yl)-3-(3- methylisothiazol-5-ylamino)pyrazine-2- carboxamide |
| 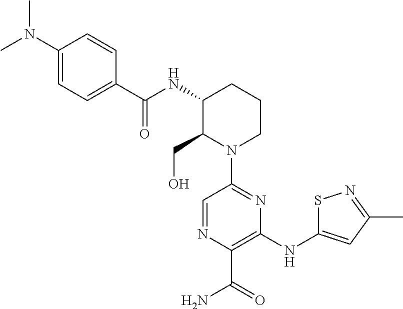 | 5-((2R,3R)-3-(4- (dimethylamino)benzamido)-2- (hydroxymethyl)piepridin-1-yl)-3-(3- methylisothiazol-5-ylamino)pyrazine-2- carboxamide |
| 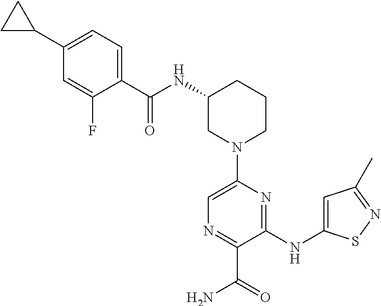 [SMILES: Cc1cc(Nc2nc(N3CCC[C@@H](NC(=O)c4ccc(C5CC5)cc4F)C3)cnc2C(N)=O)sn1] | (R)-5-(3-(4-cyclopropyl-2- fluorobenzamido)piperidin-1-yl)-3-(3- methylisothiazol-5-ylamino)pyrazine-2- carboxamide |
| 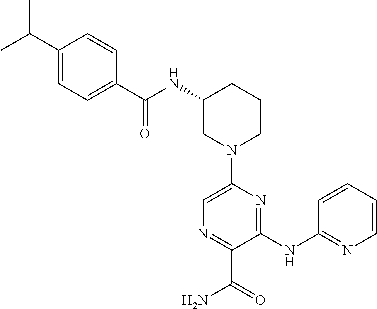 [SMILES: CC(C)c1ccc(C(=O)N[C@@H]2CCCN(c3cnc(C(N)=O)c(Nc4ccccn4)n3)C2)cc1] | (R)-5-(3-(4- isopropylbenzamido)piperidin-1-yl)-3- (pyridin-2-ylamino)pyrazine-2- carboxamide |
| 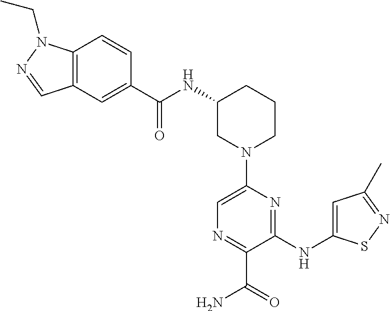 [SMILES: CCn1ncc2cc(C(=O)N[C@@H]3CCCN(c4cnc(C(N)=O)c(Nc5cc(C)ns5)n4)C3)ccc21] | (R)-N-(1-(5-carbamoyl-6-(3- methylisothiazol-5-ylamino)pyrazin-2- yl)piperidin-3-yl)-1-ethyl-1H-indazole-5- carboxamide |
| 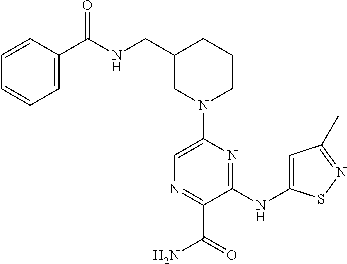 [SMILES: Cc1cc(Nc2nc(N3CCCC(CNC(=O)c4ccccc4)C3)cnc2C(N)=O)sn1] | 5-(3-(benzamidomethyl)piperidin-1-yl)- 3-(3-methylisothiazol-5- ylamino)pyrazine-2-carboxamide |
| 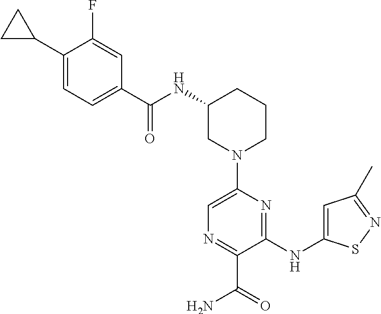 [SMILES: Cc1cc(Nc2nc(N3CCC[C@@H](NC(=O)c4ccc(C5CC5)c(F)c4)C3)cnc2C(N)=O)sn1] | (R)-5-(3-(4-cyclopropyl-3- fluorobenzamido)piperidin-1-yl)-3-(3- methylisothiazol-5-ylamino)pyrazine-2- carboxamide |
| 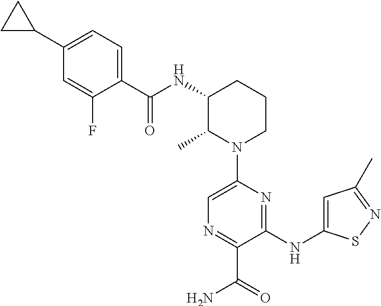 [SMILES: Cc1cc(Nc2nc(N3CCC[C@@H](NC(=O)c4ccc(C5CC5)cc4F)[C@H]3C)cnc2C(N)=O)sn1] | 5-((2R,3R)-3-(4-cyclopropyl-2- fluorobenzamido)-2-methylpiperidin-1- yl)-3-(3-methylisothiazol-5- ylamino)pyrazine-2-carboxamide |
| 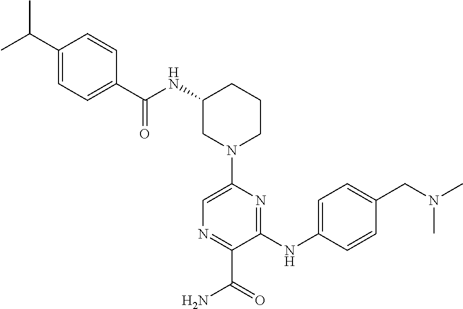 [SMILES: CC(C)c1ccc(C(=O)N[C@@H]2CCCN(c3cnc(C(N)=O)c(Nc4ccc(CN(C)C)cc4)n3)C2)cc1] | (R)-3-(4- ((dimethylamino)methyl)phenylamino)- 5-(3-(4-isopropylbenzamido)piperidin-1- yl)pyrazine-2-carboxamide |
| 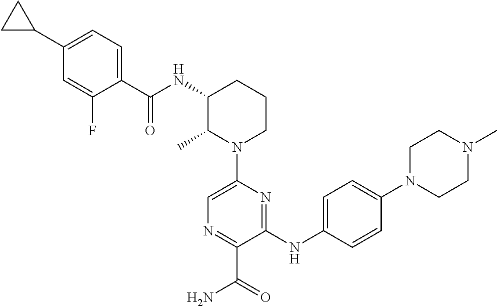 [SMILES: C[C@@H]1[C@H](NC(=O)c2ccc(C3CC3)cc2F)CCCN1c1cnc(C(N)=O)c(Nc2ccc(N3CCN(C)CC3)cc2)n1] | 5-((2R,3R)-3-(4-cyclopropyl-2- fluorobenzamido)-2-methylpiperidin-1- yl)-3-(4-(4-methylpiperazin-1- yl)phenylamino)pyrazine-2-carboxamide |
| 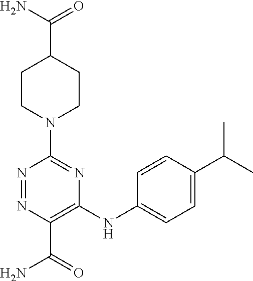 [SMILES: CC(C)c1ccc(Nc2nc(N3CCC(C(N)=O)CC3)nnc2C(N)=O)cc1] | 3-(4-carbamoylpiperidin-1-yl)-5-(4- isopropylphenylamino)-1,2,4-triazine-6- carboxamide |
| 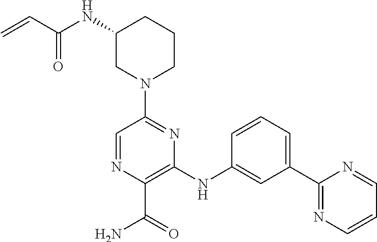 [SMILES: C=CC(=O)N[C@@H]1CCCN(c2cnc(C(N)=O)c(Nc3cccc(-c4ncccn4)c3)n2)C1] | (R)-5-(3-acrylamidopiperidin-1-yl)-3-(3- (pyrimidin-2-yl)phenylamino)pyrazine-2- carboxamide |
| 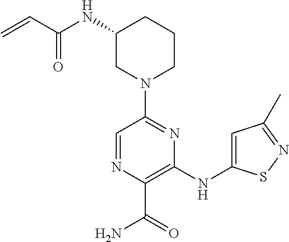 [SMILES: C=CC(=O)N[C@@H]1CCCN(c2cnc(C(N)=O)c(Nc3cc(C)ns3)n2)C1] | (R)-5-(3-acrylamidopiperidin-1-yl)-3-(3- methylisothiazol-5-ylamino)pyrazine-2- carboxamide |
| 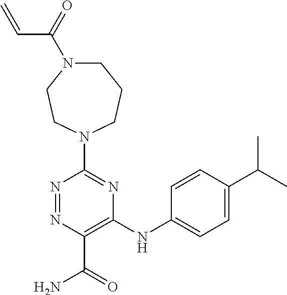 [SMILES: C=CC(=O)N1CCCN(c2nnc(C(N)=O)c(Nc3ccc(C(C)C)cc3)n2)CC1] | 3-(4-acryloyl-1,4-diazepan-1-yl)-5-(4- isopropylphenylamino)-1,2,4-triazine-6- carboxamide |
| 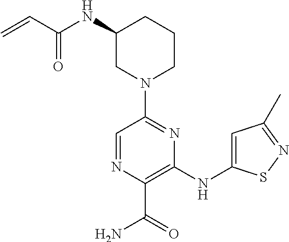 [SMILES: C=CC(=O)N[C@H]1CCCN(c2cnc(C(N)=O)c(Nc3cc(C)ns3)n2)C1] | (S)-5-(3-acrylamidopiperidin-1-yl)-3-(3- methylisothiazol-5-ylamino)pyrazine-2- carboxamide |
| 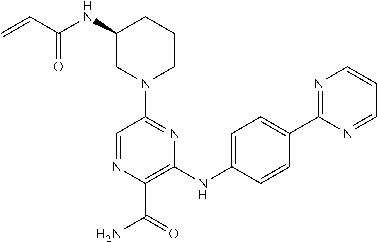 [SMILES: C=CC(=O)N[C@H]1CCCN(c2cnc(C(N)=O)c(Nc3ccc(-c4ncccn4)cc3)n2)C1] | (S)-5-(3-acrylamidopiperidin-1-yl)-3-(4- (pyrimidin-2-yl)phenylamino)pyrazine-2- carboxamide |

or a pharmaceutically acceptable solvate, pharmaceutically acceptable salt, and/or pharmaceutically acceptable prodrug thereof.

In some embodiments, the compound is selected from compounds listed in Table N2:

TABLE N2
(R)-3-(3-acrylamidopiperidin-1-yl)-5-(4-isopropylphenylamino)-1,2,4-triazine-6-carboxamide;
(S)-3-(3-acrylamidopiperidin-1-yl)-5-(4-isopropylphenylamino)-1,2,4-triazine-6-carboxamide;
5-((2R,3R)-3-(5-cyclopropylpicolinamido)-2-methylpiperidin-1-yl)-3-(5-(4-methylpiperazin-1-
yl)pyridin-2-ylamino)pyrazine-2-carboxamide;
5-[(2R,3R)-3-[(dimethylcarbamoyl)amino]-2-methylpiperidin-1-yl]-3-{[4-(4-methylpiperazine-
1-carbonyl)phenyl]amino}pyrazine-2-carboxamide;
5-[(3R)-3-(6-cyclopropyl-1-oxo-1,2-dihydroisoquinolin-2-yl)piperidin-1-yl]-3-{[4-(4-
methylpiperazin-1-yl)phenyl]amino}pyrazine-2-carboxamide;
5-[(2R,3R)-3-[(dimethylcarbamoyl)amino]-2-methylpiperidin-1-yl]-3-{[4-(4-methylpiperazin-1-
yl)phenyl]amino}pyrazine-2-carboxamide;
3-{[4-(1-cyclopentyl-4-methylpiperidin-4-yl)phenyl]amino}-5-[(2S,3R)-3-
[(dimethylcarbamoyl)amino]-2-(hydroxymethyl)piperidin-1-yl]pyrazine-2-carboxamide;
3-{[4-(1-cyclopentyl-4-methylpiperidin-4-yl)phenyl]amino}-5-[(2R,3S)-3-
[(dimethylcarbamoyl)amino]-2-(hydroxymethyl)piperidin-1-yl]pyrazine-2-carboxamide;
3-{[4-(1-cyclopentyl-4-methylpiperidin-4-yl)phenyl]amino}-5-[(2R,3R)-3-
[(dimethylcarbamoyl)amino]-2-(hydroxymethyl)piperidin-1-yl]pyrazine-2-carboxamide;
3-{[4-(1-cyclopentyl-4-methylpiperidin-4-yl)phenyl]amino}-5-[(2S,3S)-3-
[(dimethylcarbamoyl)amino]-2-(hydroxymethyl)piperidin-1-yl]pyrazine-2-carboxamide;
3-{[4-(1-cyclopentyl-4-methylpiperidin-4-yl)phenyl]amino}-5-[(2R,3R)-3-(4-
cyclopropylbenzamido)-2-methylpiperidin-1-yl]pyrazine-2-carboxamide;
5-[(2R,3R)-3-(4-cyclopropyl-2-fluorobenzamido)-2-methylpiperidin-1-yl]-3-({4-[(4-
methylpiperazin-1-yl)methyl]phenyl}amino)pyrazine-2-carboxamide;
5-[(3R)-3-(6-cyclopropyl-1-oxo-1,2-dihydroisoquinolin-2-yl)piperidin-1-yl]-3-[(3-methyl-1,2-
thiazol-5-yl)amino]pyrazine-2-carboxamide;
5-[(2R,3R)-3-(4-cyclopropyl-2-fluorobenzamido)-2-methylpiperidin-1-yl]-3-({5-methyl-
4H,5H,6H,7H-[1,3]thiazolo[5,4-c]pyridin-2-yl}amino)pyrazine-2-carboxamide;
5-[(2R,3R)-3-(4-cyclopropyl-2-fluorobenzamido)-2-methylpiperidin-1-yl]-3-[(4-methyl-1,3-
thiazol-2-yl)amino]pyrazine-2-carboxamide;
5-[(2R,3R)-3-(4-cyclopropyl-2-fluorobenzamido)-2-methylpiperidin-1-yl]-3-[(1-methyl-1H-
pyrazol-4-yl)amino]pyrazine-2-carboxamide;
5-[(3R)-3-(6-cyclopropyl-1-oxo-1,2-dihydroisoquinolin-2-yl)piperidin-1-yl]-3-({4-[(4-
methylpiperazin-1-yl)methyl]phenyl}amino)pyrazine-2-carboxamide;
5-[(3R)-3-(6-cyclopropyl-1-oxo-1,2-dihydroisoquinolin-2-yl)piperidin-1-yl]-3-[(quinolin-6-
yl)amino]pyrazine-2-carboxamide;
5-[(3R)-3-(4-cyclopropyl-2-fluorobenzamido)piperidin-1-yl]-3-[(3-methyl-1,2-oxazol-5-
yl)amino]pyrazine-2-carboxamide;
5-[(3R)-3-(4-cyclopropyl-2-fluorobenzamido)piperidin-1-yl]-3-[(3-phenyl-1,2-thiazol-5-
yl)amino]pyrazine-2-carboxamide;
5-[(3R)-3-(6-cyclopropyl-1-oxo-1,2-dihydroisoquinolin-2-yl)piperidin-1-yl]-3-{[4-(4-
methylpiperazine-1-carbonyl)phenyl]amino}pyrazine-2-carboxamide;
5-[(2R,3R)-3-(4-cyclopropyl-2-fluorobenzamido)-2-methylpiperidin-1-yl]-3-[(quinolin-6-
yl)amino]pyrazine-2-carboxamide;
5-[(2S,3S)-3-[(dimethylcarbamoyl)amino]-2-(hydroxymethyl)piperidin-1-yl]-3-[(quinolin-6-
yl)amino]pyrazine-2-carboxamide;
5-[(2R,3S)-3-[(dimethylcarbamoyl)amino]-2-(hydroxymethyl)piperidin-1-yl]-3-[(quinolin-6-
yl)amino]pyrazine-2-carboxamide;
5-[(2R,3R)-3-{2-fluoro-4-[(1E)-prop-1-en-1-yl]benzamido}-2-methylpiperidin-1-yl]-3-[(3-
methyl-1,2-thiazol-5-yl)amino]pyridine-2-carboxamide;
5-[(3R)-3-(4-cyclopropyl-2-fluorobenzamido)piperidin-1-yl]-3-[(2-methyl-1,3-thiazol-5-
yl)amino]pyrazine-2-carboxamide;
3-{[4-(1-cyclopentyl-4-methylpiperidin-4-yl)phenyl]amino}-5-[(2R,3R)-3-(4-cyclopropyl-2-
fluorobenzamido)-2-methylpiperidin-1-yl]pyrazine-2-carboxamide;
5-[(2R,3R)-3-(4-cyclopropyl-2-fluorobenzamido)-2-methylpiperidin-1-yl]-3-{[4-(4-
methylpiperazine-1-carbonyl)phenyl]amino}pyrazine-2-carboxamide;
5-[(2S,3R)-3-[(dimethylcarbamoyl)amino]-2-(hydroxymethyl)piperidin-1-yl]-3-[(3-methyl-1,2-
thiazol-5-yl)amino]pyrazine-2-carboxamide;
5-[(2R,3S)-3-[(dimethylcarbamoyl)amino]-2-(hydroxymethyl)piperidin-1-yl]-3-[(3-methyl-1,2-
thiazol-5-yl)amino]pyrazine-2-carboxamide;
3-{[4-(4-cyclopentylpiperazin-1-yl)phenyl]amino}-5-[(2R,3R)-3-(4-cyclopropyl-2-
fluorobenzamido)-2-methylpiperidin-1-yl]pyrazine-2-carboxamide;
5-[(2R,3R)-3-(4-cyclopropyl-2-fluorobenzamido)-2-methylpiperidin-1-yl]-3-[(3-methyl-1,2-
thiazol-5-yl)amino]pyridine-2-carboxamide;
5-[(2R,3R)-3-(4-cyclopropyl-2-fluorobenzamido)-2-methylpiperidin-1-yl]-3-[(1-methyl-1H-
pyrazol-4-yl)amino]pyridine-2-carboxamide;
5-[(2R,3R)-3-(4-cyclopropyl-2-fluorobenzamido)-2-methylpiperidin-1-yl]-3-{[3-(morpholin-4-
ylmethyl)-1,2-thiazol-5-yl]amino}pyrazine-2-carboxamide;
5-[(2R,3R)-3-[(dimethylcarbamoyl)amino]-2-methylpiperidin-1-yl]-3-{[3-(morpholin-4-
ylmethyl)-1,2-thiazol-5-yl]amino}pyrazine-2-carboxamide;
3-[(dimethyl-1,2-thiazol-5-yl)amino]-5-[(2R,3R)-3-[(dimethylcarbamoyl)amino]-2-
methylpiperidin-1-yl]pyrazine-2-carboxamide;
5-[(2R,3R)-3-[(dimethylcarbamoyl)amino]-2-methylpiperidin-1-yl]-3-({4-[(4-methylpiperazin-
1-yl)methyl]phenyl}amino)pyridine-2-carboxamide;
5-[(2R,3R)-3-[(dimethylcarbamoyl)amino]-2-methylpiperidin-1-yl]-3-[(quinolin-7-
yl)amino]pyrazine-2-carboxamide;
5-[(3R)-3-(6-cyclopropyl-8-fluoro-1-oxo-1,2-dihydroisoquinolin-2-yl)piperidin-1-yl]-3-{[4-(4-
methylpiperazin-1-yl)phenyl]amino}pyrazine-2-carboxamide;
5-[(2R,3R)-3-(4-cyclopropyl-2-fluorobenzamido)-2-methylpiperidin-1-yl]-3-({3-
[(dimethylamino)methyl]-1,2-thiazol-5-yl}amino)pyrazine-2-carboxamide;
5-[(2R,3R)-3-[(dimethylcarbamoyl)amino]-2-methylpiperidin-1-yl]-3-[(3-methyl-1,2-thiazol-5-
yl)amino]pyridine-2-carboxamide;
5-[(2R,3R)-3-(4-cyclopropyl-2-fluorobenzamido)-2-methylpiperidin-1-yl]-3-{[3-(piperidin-1-
ylmethyl)-1,2-thiazol-5-yl]amino}pyrazine-2-carboxamide;
5-[(2R,3R)-3-(4-cyclopropyl-2-fluorobenzamido)-2-methylpiperidin-1-yl]-3-{[1-(propan-2-yl)-
1H-pyrazol-4-yl]amino}pyrazine-2-carboxamide;
5-[(2R,3R)-3-(4-cyclopropyl-2-fluorobenzamido)-2-methylpiperidin-1-yl]-3-[(quinolin-7-
yl)amino]pyrazine-2-carboxamide;
5-[(2R,3R)-3-(4-cyclopropyl-2-fluorobenzamido)-2-methylpiperidin-1-yl]-3-({4-[(4-
methylpiperazin-1-yl)methyl]phenyl}amino)pyridine-2-carboxamide;
5-[(2R,3R)-3-(6-cyclopropyl-1-oxo-1,2-dihydroisoquinolin-2-yl)-2-methylpiperidin-1-yl]-3-{[4-
(4-methylpiperazin-1-yl)phenyl]amino}pyrazine-2-carboxamide;
3-{[4-(1-cyclopentyl-4-methylpiperidin-4-yl)phenyl]amino}-5-[(3R)-3-(6-cyclopropyl-1-oxo-
1,2-dihydroisoquinolin-2-yl)piperidin-1-yl]pyrazine-2-carboxamide;
5-[(2R,3R)-3-(4-cyclopropyl-2-fluorobenzamido)-2-methylpiperidin-1-yl]-3-{[4-(4-
methylpiperazin-1-yl)phenyl]amino}pyridine-2-carboxamide;
5-[(2R,3R)-3-[(dimethylcarbamoyl)amino]-2-methylpipendin-1-yl]-3-{[4-(4-methylpiperazin-1-
yl)phenyl]amino}pyridine-2-carboxamide;
(R)-5-(3-(6-cyclopropyl-1-oxoisoquinolin-2(1H)-yl)piperidin-1-yl)-3-(4-(4-methylpiperazin-1-
yl)phenylamino)picolinamide;
5-[(3R)-3-(6-cyclopropyl-1-oxo-1,2-dihydroisoquinolin-2-yl)piperidin-1-yl]-3-{[5-(4-
methylpiperazin-1-yl)pyridin-2-yl]amino}pyrazine-2-carboxamide;
5-[(2R,3R)-3-[(dimethylcarbamoyl)amino]-2-methylpiperidin-1-yl]-3-[(1-methyl-1H-pyrazol-4-
yl)amino]pyrazine-2-carboxamide;
5-[(2R,3R)-3-[(dimethylcarbamoyl)amino]-2-methylpiperidin-1-yl]-3-{[5-(4-methylpiperazin-1-
yl)pyridin-2-yl]amino}pyrazine-2-carboxamide;
5-[(2R,3R)-3-(4-cyclopropyl-2-fluorobenzamido)-2-methylpiperidin-1-yl]-3-{[4-(morpholin-4-
yl)phenyl]amino}pyrazine-2-carboxamide;
5-[(2R,3R)-3-(4-cyclopropyl-2-fluorobenzamido)-2-methylpiperidin-1-yl]-3-({4-[4-(propan-2-
yl)piperazin-1-yl]phenyl}amino)pyrazine-2-carboxamide;
5-[(2R,3R)-3-(4-cyclopropyl-2-fluorobenzamido)-2-methylpiperidin-1-yl]-3-{[5-(4-
methylpiperazin-1-yl)pyridin-2-yl]amino}pyrazine-2-carboxamide;
5-[(2S,5R)-5-[(dimethylcarbamoyl)amino]-2-methylpiperidin-1-yl]-3-[(3-methyl-1,2-thiazol-5-
yl)amino]pyrazine-2-carboxamide;
5-[(2R,5S)-5-[(dimethylcarbamoyl)amino]-2-methylpiperidin-1-yl]-3-[(3-methyl-1,2-thiazol-5-
yl)amino]pyrazine-2-carboxamide;
5-[(2R,5R)-5-[(dimethylcarbamoyl)amino]-2-methylpiperidin-1-yl]-3-[(3-methyl-1,2-thiazol-5-
yl)amino]pyrazine-2-carboxamide;
5-[(2S,5S)-5-[(dimethylcarbamoyl)amino]-2-methylpiperidin-1-yl]-3-[(3-methyl-1,2-thiazol-5-
yl)amino]pyrazine-2-carboxamide;
5-[(2R,3R)-3-(4-cyclopropyl-2-fluorobenzamido)-2-methylpiperidin-1-yl]-3-[(1-methyl-1H-
pyrazol-3-yl)amino]pyrazine-2-carboxamide;
5-[(2R,3R)-3-(4-cyclopropyl-2-fluorobenzamido)-2-methylpiperidin-1-yl]-3-({4-[(4-
methylpiperazin-1-yl)sulfonyl]phenyl}amino)pyrazine-2-carboxamide;
5-[(2R,5R)-5-(4-cyclopropyl-2-fluorobenzamido)-2-methylpiperidin-1-yl]-3-[(3-methyl-1,2-
thiazol-5-yl)amino]pyrazine-2-carboxamide;
5-[(2S,5S)-5-(4-cyclopropyl-2-fluorobenzamido)-2-methylpiperidin-1-yl]-3-[(3-methyl-1,2-
thiazol-5-yl)amino]pyrazine-2-carboxamide;
5-[(2R,5S)-5-(4-cyclopropyl-2-fluorobenzamido)-2-methylpiperidin-1-yl]-3-[(3-methyl-1,2-
thiazol-5-yl)amino]pyrazine-2-carboxamide;
5-[(2S,5R)-5-(4-cyclopropyl-2-fluorobenzamido)-2-methylpiperidin-1-yl]-3-[(3-methyl-1,2-
thiazol-5-yl)amino]pyrazine-2-carboxamide;
3-{[4-(4-cyclopentylpiperazin-1-yl)phenyl]amino}-5-[(2R,3R)-3-[(dimethylcarbamoyl)amino]-
2-methylpiperidin-1-yl]pyrazine-2-carboxamide;
3-{[4-(1-cyclopentyl-4-methylpiperidin-4-yl)phenyl]amino}-5-[(2R,3R)-2-methyl-3-{[(pyridin-
3-yl)carbamoyl]amino}piperidin-1-yl]pyrazine-2-carboxamide;
5-[(2R,3R)-3-(4-cyclopropyl-2-fluorobenzamido)-2-methylpiperidin-1-yl]-3-{[4-(4-methyl-2-
oxopiperazin-1-yl)phenyl]amino}pyrazine-2-carboxamide;
5-[(2R,3R)-3-(4-cyclopropyl-2-fluorobenzamido)-2-methylpiperidin-1-yl]-3-({5-[(4-
methylpiperazin-1-yl)methyl]pyridin-2-yl}amino)pyrazine-2-carboxamide;
5-[(2R,3R)-3-(4-cyclopropyl-2-fluorobenzamido)-2-methylpiperidin-1-yl]-3-[(4-
methanesulfonylphenyl)amino]pyrazine-2-carboxamide;
3-{[4-(1-cyclopentyl-4-methylpiperidin-4-yl)phenyl]amino}-5-[(2R,3R)-3-[4-
(dimethylamino)benzamido]-2-methylpiperidin-1-yl]pyrazine-2-carboxamide;
5-[(3R)-3-(6-cyclopropyl-1-oxo-1,2-dihydroisoquinolin-2-yl)piperidin-1-yl]-3-[(1-methyl-1H-
pyrazol-4-yl)amino]pyrazine-2-carboxamide;
3-{[4-(1-cyclobutyl-4-methylpiperidin-4-yl)phenyl]amino}-5-[(2R,3R)-3-(4-cyclopropyl-2-
fluorobenzamido)-2-methylpiperidin-1-yl]pyrazine-2-carboxamide;
5-[(2R,3R)-3-(4-cyclopropylbenzamido)-2-methylpiperidin-1-yl]-3-[(1-methyl-1H-pyrazol-4-
yl)amino]pyrazine-2-carboxamide;
5-[(2R,3R)-3-(4-cyclopropylbenzamido)-2-methylpiperidin-1-yl]-3-{[4-(4-methylpiperazin-1-
yl)phenyl]amino}pyrazine-2-carboxamide;
5-[(2R,3R)-3-(4-cyclopropyl-2-fluorobenzamido)-2-methylpiperidin-1-yl]-3-{[3-fluoro-4-(4-
methylpiperazin-1-yl)phenyl]amino}pyrazine-2-carboxamide;
5-[(2R,3R)-2-methyl-3-[4-(trifluoromethyl)benzamido]piperidin-1-yl]-3-{[4-(4-
methylpiperazin-1-yl)phenyl]amino}pyrazine-2-carboxamide;
5-[(2R,3R)-3-(4-cyclopropylbenzamido)-2-methylpiperidin-1-yl]-3-{[1-(1-methylpiperidin-4-
yl)-1H-pyrazol-4-yl]amino}pyrazine-2-carboxamide;
5-[(2R,3R)-3-(4-cyclopropyl-2-fluorobenzamido)-2-methylpiperidin-1-yl]-3-{[4-(1-cyclopropyl-
4-methylpiperidin-4-yl)phenyl]amino}pyrazine-2-carboxamide;
5-[(2R,3R)-3-(4-cyclopropyl-2-fluorobenzamido)-2-methylpiperidin-1-yl]-3-{[4-(pyrrolidine-1-
sulfonyl)phenyl]amino}pyrazine-2-carboxamide;
5-[(2R,3R)-3-(4-cyclopropyl-2-fluorobenzamido)-2-methylpiperidin-1-yl]-3-({4-[(2S)-2,4-
dimethylpiperazin-1-yl]phenyl}amino)pyrazine-2-carboxamide;
5-[(2R,3R)-3-(4-cyclopropyl-2-fluorobenzamido)-2-methylpiperidin-1-yl]-3-{[6-(4-
methylpiperazin-1-yl)pyridin-3-yl]amino}pyrazine-2-caoxamide;
5-[(3R)-3-(6-cyclopropyl-1-oxo-1,2-dihydroisoquinolin-2-yl)piperidin-1-yl]-3-{[1-(1-
methylpiperidin-4-yl)-1H-pyrazol-4-yl]amino}pyrazine-2-carboxamide;
5-[(2R,3R)-3-(5-cyclopropylpyridine-2-amido)-2-methylpiperidin-1-yl]-3-{[4-(4-
methylpiperazin-1-yl)phenyl]amino}pyrazine-2-carboxamide;
5-[(2R,3R)-3-(2-chloro-4-cyclopropylbenzamido)-2-methylpiperidin-1-yl]-3-{[4-(4-
methylpiperazin-1-yl)phenyl]amino}pyrazine-2-carboxamide;
5-[(2R,3R)-3-(4-cyclopropylbenzamido)-2-methylpiperidin-1-yl]-3-{[3-(piperidin-1-ylmethyl)-
1,2-thiazol-5-yl]amino}pyrazine-2-carboxamide;
5-[(2R,3R)-3-(4-cyclopropylbenzamido)-2-methylpiperidin-1-yl]-3-({1-[2-
(dimethylamino)ethyl]-1H-pyrazol-4-yl}amino)pyrazine-2-carboxamide;
N-[(3R)-1-{5-carbamoyl-6-[(3-methyl-1,2-thiazol-5-yl)amino]pyrazin-2-yl}piperidin-3-yl]-1-
oxo-2,3-dihydro-1H-isoindole-2-carboxamide;
5-[(2R,3R)-3-(4-tert-butylbenzamido)-2-methylpiperidin-1-yl]-3-{[4-(4-methylpiperazin-1-
yl)phenyl]amino}pyrazine-2-carboxamide;
5-((2R,3R)-3-(4-cyclopropylbenzamido)-2-methylpiperidin-1-yl)-3-(3-(((2-
methoxyethyl)(methyl)amino)methyl)isothiazol-5-ylamino)pyrazine-2-carboxamide;
5-((2R,3R)-3-(4-cyclopropylbenzamido)-2-methylpiperidin-1-yl)-3-(1-(2-methoxyethyl)-1H-
pyrazol-4-ylamino)pyrazine-2-carboxamide;
(S)-5-(5-(4-cyclopropylbenzamido)-3,3-difluoropiperidin-1-yl)-3-(1-methyl-1H-pyrazol-4-
ylamino)pyrazine-2-carboxamide;
(R)-5-(5-(4-cyclopropylbenzamido)-3,3-difluoropiperidin-1-yl)-3-(1-methyl-1H-pyrazol-4-
ylamino)pyrazine-2-carboxamide
5-((2R,3R)-3-(3-chloro-4-cyclopropylbenzamido)-2-methylpiperidin-1-yl)-3-(4-(4-
methylpiperazin-1-yl)phenylamino)pyrazine-2-carboxamide;
5-((2R,3R)-3-(6-cyclopropylnicotinamido)-2-methylpiperidin-1-yl)-3-(1-methyl-1H-pyrazol-4-
ylamino)pyrazine-2-carboxamide;
5-((2R,3R)-3-(4-cyclopropylbenzamido)-2-methylpiperidin-1-yl)-3-(1,5-dimethyl-1H-pyrazol-4-
ylamino)pyrazine-2-carboxamide;
5-((2R,3R)-3-(4-cyclopropylbenzamido)-2-methylpiperidin-1-yl)-3-(1,3-dimethyl-1H-pyrazol-4-
ylamino)pyrazine-2-carboxamide;
N-((2R,3R)-1-(5-carbamoyl-6-(1-methyl-1H-pyrazol-4-ylamino)pyrazin-2-yl)-2-
methylpiperidin-3-yl)-1,5,5-trimethyl-1,4,5,6-tetrahydrocyclopenta[b]pyrrole-2-carboxamide;
5-((2R,3R)-3-(2-chloro-4-cyclopropylbenzamido)-2-methylpiperidin-1-yl)-3-(1-methyl-1H-
pyrazol-4-ylamino)pyrazine-2-carboxamide;
5-((2R,3R)-3-acrylamido-2-methylpiperidin-1-yl)-3-(1-methyl-1H-pyrazol-4-ylamino)pyrazine-
2-carboxamide;
5-((2R,3R)-3-acrylamido-2-methylpiperidin-1-yl)-3-(3-methylisothiazol-5-ylamino)pyrazine-2-
carboxamide;
5-((2R,3R)-3-acrylamido-2-methylpiperidin-1-yl)-3-(4-(1-cyclopropylpiperidin-4-
yl)phenylamino)pyrazine-2-carboxamide;
5-[(2R,3R)-3-(4-cyclopropylbenzamido)-2-methylpiperidin-1-yl]-3-{[1-(difluoromethyl)-1H-
pyrazol-4-yl]amino}pyrazine-2-carboxamide;
5-[(2R,3R)-3-(4-cyclopropylbenzamido)-2-methylpiperidin-1-yl]-3-{[1-methyl-5-
(trifluoromethyl)-1H-pyrazol-4-yl]amino}pyrazine-2-carboxamide;
5-((2R,3R)-3-(4-(2-hydroxypropan-2-yl)benzamido)-2-methylpiperidin-1-yl)-3-(1-methyl-1H-
pyrazol-4-ylamino)pyrazine-2-carboxamide;
3-[(2R,3R)-3-(4-cyclopropylbenzamido)-2-methylpiperidin-1-yl]-5-[(1-methyl-1H-pyrazol-4-
yl)amino]-1,2,4-triazine-6-carboxamide;
3-[(3R)-3-(4-cyclopropylbenzamido)piperidin-1-yl]-5-[(1-methyl-1H-pyrazol-4-yl)amino]-1,2,4-
triazine-6-carboxamide;
5-((2R,3R)-3-(4-tert-butylbenzamido)-2-methylpiperidin-1-yl)-3-(1-methyl-1H-pyrazol-4-
ylamino)pyrazine-2-carboxamide;
5-[(2R,3R)-3-(4-cyclopropylbenzamido)-2-methylpiperidin-1-yl]-3-{[1-methyl-3-
(trifluoromethyl)-1H-pyrazol-4-yl]amino}pyrazine-2-carboxamide;
3-[(1-cyclopropyl-1H-pyrazol-4-yl)amino]-5-[(2R,3R)-3-(4-cyclopropylbenzamido)-2-
methylpiperidin-1-yl]pyrazine-2-carboxamide;
3-(1-methyl-1H-pyrazol-4-ylamino)-5-((2R,3R)-2-methyl-3-(4-(oxetan-3-
yl)benzamido)piperidin-1-yl)pyrazine-2-carboxamide;
5-[(2R,3R)-3-(5-cyclopropylpyrimidine-2-amido)-2-methylpiperidin-1-yl]-3-[(1-methyl-1H-
pyrazol-4-yl)amino]pyrazine-2-carboxamide;
5-[(2R,3R)-3-(5-cyclopropylpyrazine-2-amido)-2-methylpiperidin-1-yl]-3-[(1-methyl-1H-
pyrazol-4-yl)amino]pyrazine-2-carboxamide;
3-[(1-methyl-1H-pyrazol-4-yl)amino]-5-[(2R,3R)-2-methyl-3-(3-methyl-2-oxoimidazolidin-1-
yl)piperidin-1-yl]pyrazine-2-carboxamide;
5-((2R,3R)-3-(5-cyclopropylpicolinamido)-2-methylpiperidin-1-yl)-3-(4-(4-methylpiperazin-1-
yl)phenylamino)pyrazine-2-carboxamide;
5-((2R,3R)-3-(6-cyclopropylnicotinamido)-2-methylpiperidin-1-yl)-3-(4-(4-methylpiperazin-1-
yl)phenylamino)pyrazine-2-carboxamide;
5-((2R,3R)-3-(4-tert-butylbenzamido)-2-methylpiperidin-1-yl)-3-(4-(4-methylpiperazin-1-
yl)phenylamino)pyrazine-2-carboxamide;
5-((2R,3R)-3-(4-(dimethylamino)benzamido)-2-methylpiperidin-1-yl)-3-(4-(4-methylpiperazin-
1-yl)phenylamino)pyrazine-2-carboxamide;
5-((2R,3R)-3-(6-cyclopropylnicotinamido)-2-methylpiperidin-1-yl)-3-(5-(4-methylpiperazin-1-
yl)pyridin-2-ylamino)pyrazine-2-carboxamide;
5-((2R,3R)-3-(4-(dimethylamino)benzamido)-2-memylpiperidin-1-yl)-3-(5-(4-methylpiperazin-
1-yl)pyridin-2-ylamino)pyrazine-2-carboxamide;
5-((2R,3R)-3-(4-tert-butylbenzamido)-2-methylpiperidin-1-yl)-3-(5-(4-methylpiperazin-1-
yl)pyridin-2-ylamino)pyrazine-2-carboxamide;
tert-butyl 4-(4-(3-carbamoyl-6-((2R,3R)-3-(4-cyclopropylbenzamido)-2-methylpiperidin-1-
yl)pyrazin-2-ylamino)-1H-pyrazol-1-yl)piperidine-1-carboxylate;
5-((2R,3R)-3-(4-cyclopropylbenzamido)-2-methylpiperidin-1-yl)-3-(1-(piperidin-4-yl)-1H-
pyrazol-4-ylamino)pyrazine-2-carboxamide;
3-{[1-(1-acetylpiperidin-4-yl)-1H-pyrazol-4-yl]amino}-5-[(2R,3R)-3-(4-
cyclopropylbenzamido)-2-methylpiperidin-1-yl]pyrazine-2-carboxamide;
5-((2R,3R)-3-(4-cyclopropylbenzamido)-2-methylpiperidin-1-yl)-3-(4-(4-methyl-3-
oxopiperazin-1-yl)phenylamino)pyrazine-2-carboxamide;
5-((2R,3R)-3-(4-cyclopropylbenzamido)-2-methylpiperidin-1-yl)-3-(3-fluoro-4-(4-
methylpiperazin-1-yl)phenylamino)pyrazine-2-carboxamide;
5-((2R,3R)-3-(4-cyclopropylbenzamido)-2-methylpiperidin-1-yl)-3-(4-(4,4-difluoropiperidin-1-
yl)phenylamino)pyrazine-2-carboxamide;
5-((2R,3R)-3-(4-cyclopropylbenzamido)-2-methylpiperidin-1-yl)-3-(4-(1-methylpiperidin-4-
yloxy)phenylamino)pyrazine-2-carboxamide;
5-((2R)-2-(4-cyclopropylbenzamido)-8-azabicyclo[3.2.1]octan-8-yl)-3-(1-methyl-1H-pyrazol-4-
ylamino)pyrazine-2-carboxamide;
5-((2S)-2-(4-cyclopropylbenzamido)-8-azabicyclo[3.2.1]octan-8-yl)-3-(1-methyl-1H-pyrazol-4-
ylamino)pyrazine-2-carboxamide;
5-[(3S,4S)-3-(4-cyclopropylbenzamido)-4-hydroxypiperidin-1-yl]-3-[(1-methyl-1H-pyrazol-4-
yl)amino]pyrazine-2-carboxamide;
3-(1H-pyrazol-4-ylamino)-5-((2R,3R)-3-(4-cyclopropylbenzamido)-2-methylpiperidin-1-
yl)pyrazine-2-carboxamide;
5-((2R,3R)-3-(4-cyclopropyl-2-(trifluoromethyl)benzamido)-2-methylpiperidin-1-yl)-3-(1-
methyl-1H-pyrazol-4-ylamino)pyrazine-2-carboxamide;
5-((2R,3R)-3-(5-cyclopropylpicolinamido)-2-methylpiperidin-1-yl)-3-(1-methyl-1H-pyrazol-4-
ylamino)pyrazine-2-carboxamide;
3-(1-methyl-1H-pyrazol-4-ylamino)-5-((2R,3R)-2-methyl-3-(4-(pyrimidin-2-
yl)benzamido)piperidin-1-yl)pyrazine-2-carboxamide;
5-((2R,3R)-3-(4-(dimethylamino)benzamido)-2-methylpiperidin-1-yl)-3-(1-methyl-1H-pyrazol-
4-ylamino)pyrazine-2-carboxamide;
5-((2R,3R)-3-(5-(dimethylamino)picolinamido)-2-methylpiperidin-1-yl)-3-(1-methyl-1H-
pyrazol-4-ylamino)pyrazine-2-carboxamide; and
5-((2R,3R)-3-(4-isopropylbenzamido)-2-methylpiperidin-1-yl)-3-(1-methyl-1H-pyrazol-4-
ylamino)pyrazine-2-carboxamide;

or a pharmaceutically acceptable solvate, pharmaceutically acceptable salt, and/or pharmaceutically acceptable prodrug thereof.

In some embodiments, the compound is selected from:

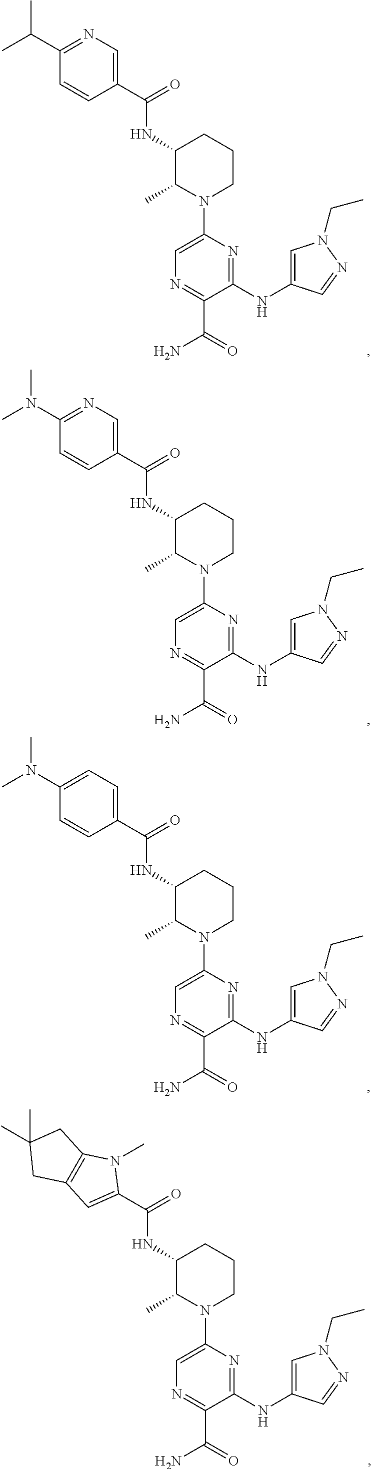

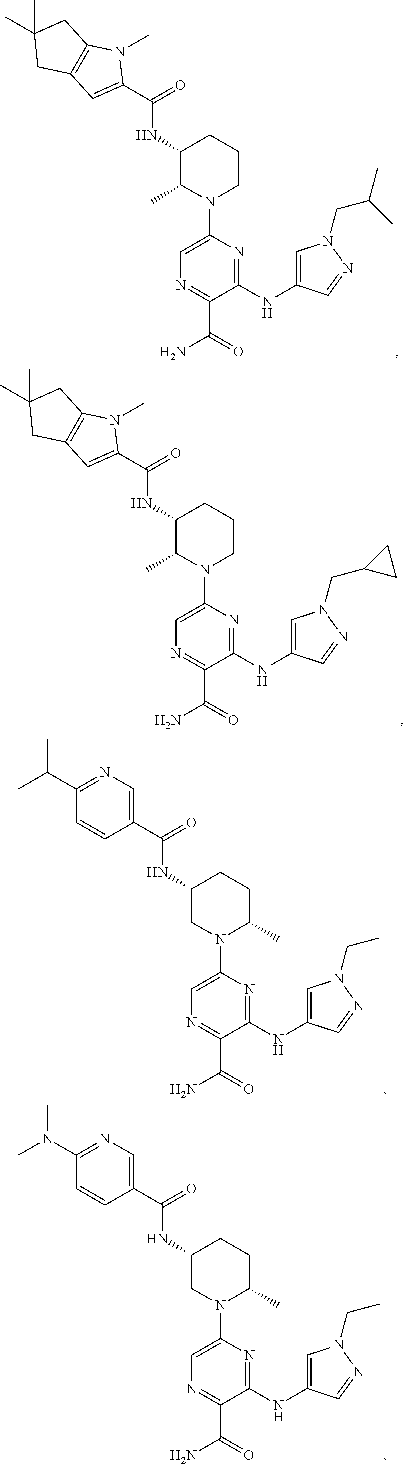

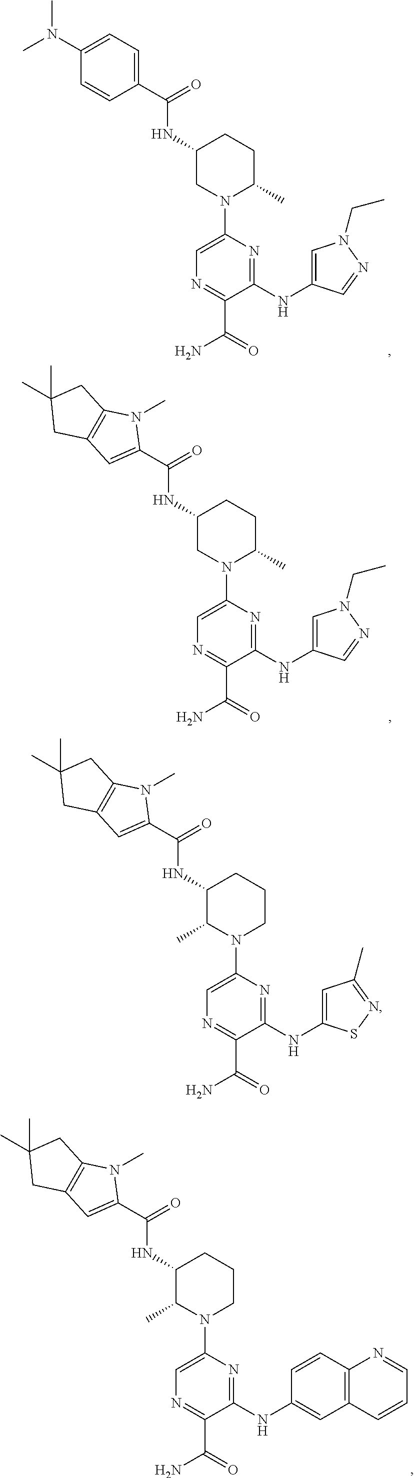

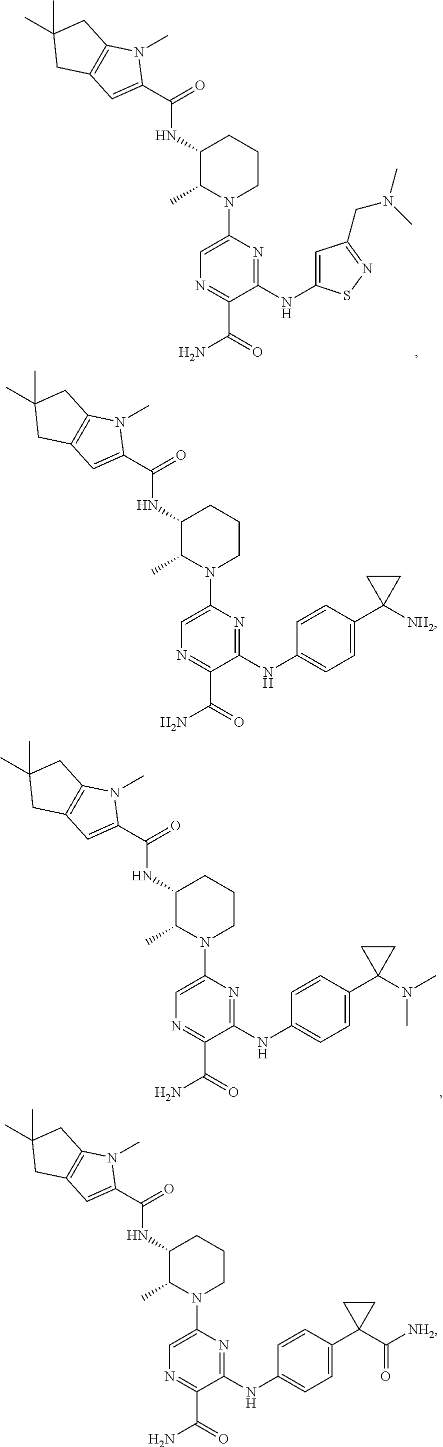

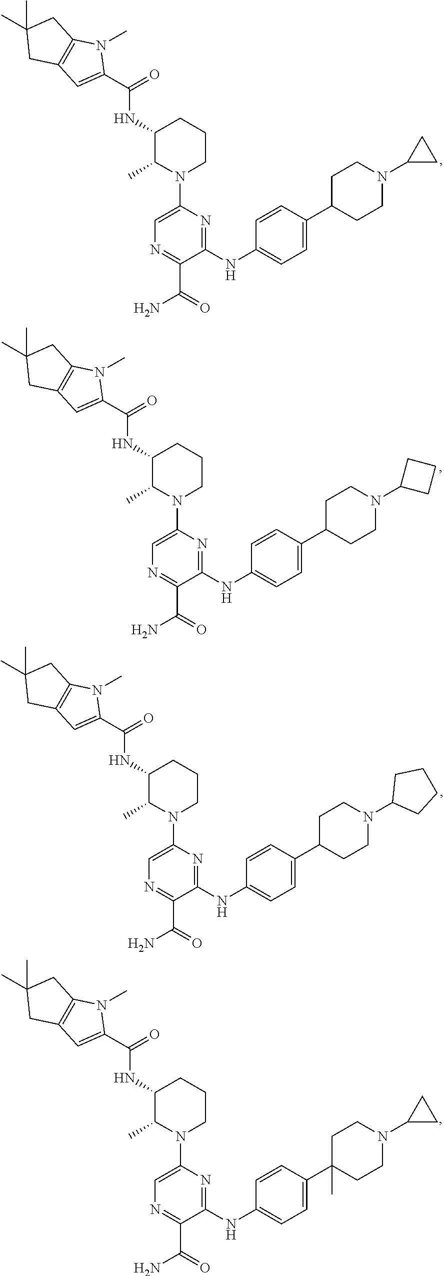 [SMILES: C[C@@H]1[C@H](NC(=O)c2cc3c(n2C)CC(C)(C)C3)CCCN1c1cnc(C(N)=O)c(Nc2ccc(C3CCN(C4CC4)CC3)cc2)n1 ; C[C@@H]1[C@H](NC(=O)c2cc3c(n2C)CC(C)(C)C3)CCCN1c1cnc(C(N)=O)c(Nc2ccc(C3CCN(C4CCC4)CC3)cc2)n1 ; C[C@@H]1[C@H](NC(=O)c2cc3c(n2C)CC(C)(C)C3)CCCN1c1cnc(C(N)=O)c(Nc2ccc(C3CCN(C4CCCC4)CC3)cc2)n1 ; C[C@@H]1[C@H](NC(=O)c2cc3c(n2C)CC(C)(C)C3)CCCN1c1cnc(C(N)=O)c(Nc2ccc(C3(C)CCN(C4CC4)CC3)cc2)n1]

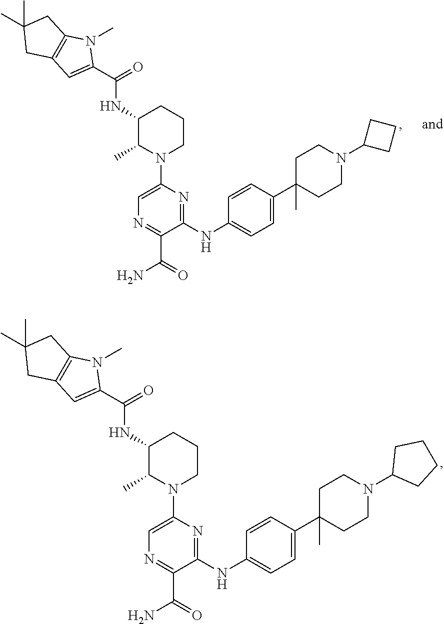 [SMILES: C[C@@H]1[C@H](NC(=O)c2cc3c(n2C)CC(C)(C)C3)CCCN1c1cnc(C(N)=O)c(Nc2ccc(C3(C)CCN(C4CCC4)CC3)cc2)n1 ; C[C@@H]1[C@H](NC(=O)c2cc3c(n2C)CC(C)(C)C3)CCCN1c1cnc(C(N)=O)c(Nc2ccc(C3(C)CCN(C4CCCC4)CC3)cc2)n1]

or a pharmaceutically acceptable solvate, pharmaceutically acceptable salt, and/or pharmaceutically acceptable prodrug thereof.

### Formula B-I Compounds

In particular embodiments, the compound is selected from the group consisting of compounds listed in Table N3:

TABLE N3
Formula B-I Compounds

| Compound Structure | Compound Name |
|---|---|
| 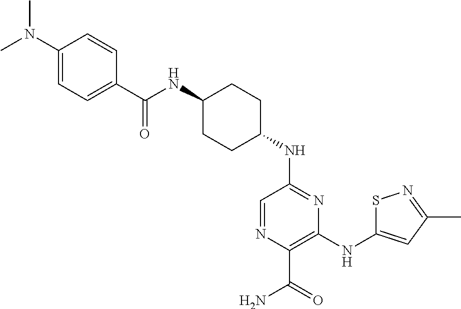 | 5-((1r,4r)-4-(4-(dimethyl- amino)benzamido)cyclohexyl- amino)-3-(3-methylisothiazol-5- ylamino)pyrazine-2-carboxamide |
| 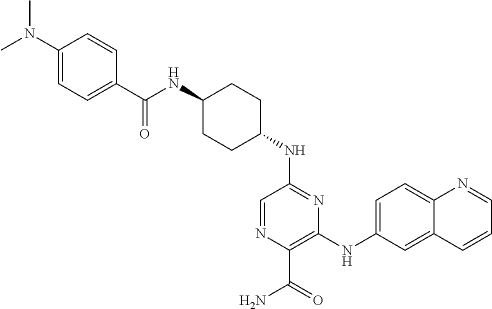 [SMILES: CN(C)c1ccc(C(=O)N[C@H]2CC[C@H](Nc3cnc(C(N)=O)c(Nc4ccc5ncccc5c4)n3)CC2)cc1] | 5-((1r,4r)-4-(4- (dimethylamino)benzamido)cyclo- hexylamino)-3-(quinolin-6- ylamino)pyrazine-2-carboxamide |
| 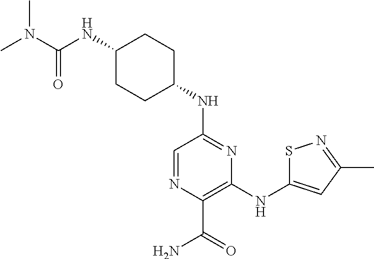 | 5-((1s,4s)-4-(3,3- dimethylureido)cyclohexylamino)-3- (3-methylisothiazol-5- ylamino)pyrazine-2-carboxamide |
| 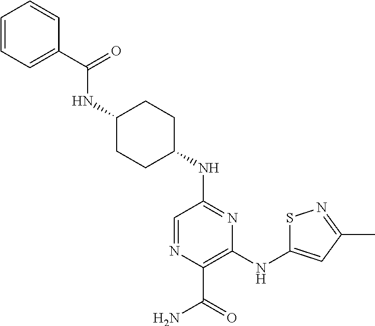 [SMILES: Cc1cc(Nc2nc(N[C@H]3CC[C@@H](NC(=O)c4ccccc4)CC3)cnc2C(N)=O)sn1] | 5-((1s,4s)-4- benzamidocyclohexylamino)-3-(3- methylisothiazol-5-ylamino)pyrazine- 2-carboxamide |
| 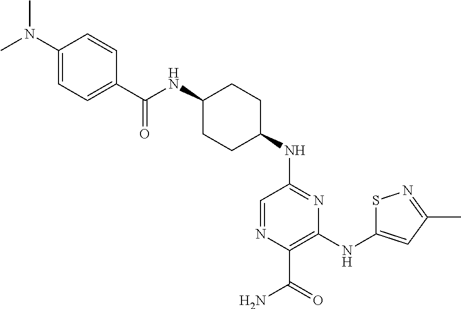 | 5-((1s,4s)-4-(4- (dimethylamino)benzamido)cyclohexyl amino)-3-(3-methylisothiazol-5- ylamino)pyrazine-2-carboxamide |
| 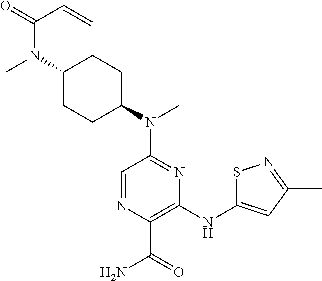 | 5-(methyl((1r,4r)-4-(N- methylacrylamido)cyclohexyl)amino)- 3-(3-methylisothiazol-5- ylamino)pyrazine-2-carboxamide |

or a pharmaceutically acceptable solvate, pharmaceutically acceptable salt, or pharmaceutically acceptable prodrug thereof.

In particular embodiments, the compound is selected from the group consisting of compounds listed in Table N4:

TABLE N4
3-((1R,2S)-2-acrylamidocyclohexylamino)-5-(4-isopropylphenylamino)-1,2,4-triazine-6-
carboxamide;
3-((1R,2R)-2-acrylamidocyclohexylamino)-5-(4-isopropylphenylamino)-1,2,4-triazine-6-
carboxamide;
3-((1S,2S)-2-acrylamidocyclohexylamino)-5-(4-isopropylphenylamino)-1,2,4-triazine-6-
carboxamide;
3-((1S,2R)-2-acrylamidocyclohexylamino)-5-(4-isopropylphenylamino)-1,2,4-triazine-6-
carboxamide;
3-((1R,3R)-3-acrylamidocyclohexylamino)-5-(4-isopropylphenylamino)-1,2,4-triazine-6-
carboxamide;
3-((1R,3S)-3-acrylamidocyclohexylamino)-5-(4-isopropylphenylamino)-1,2,4-triazine-6-
carboxamide;
5-(4-(3,3-dimethylureido)cyclohexylamino)-3-(4-(4-methylpiperazine-1-
carbonyl)phenylamino)pyrazine-2-carboxamide;
5-((1r,4r)-4-(4-cyclopropyl-2-fluorobenzamido)cyclohexylamino)-3-(4-(4-methylpiperazine-1-
carbonyl)phenylamino)pyrazine-2-carboxamide;
5-(4-(4-cyclopropyl-2-fluorobenzamido)cyclohexylamino)-3-(4-(4-methylpiperazine-1-
carbonyl)phenylamino)pyrazine-2-carboxamide;
5-((1s,4s)-4-(3,3-dimethylureido)cyclohexylamino)-3-(4-(4-methylpiperazine-1-
carbonyl)phenylamino)pyrazine-2-carboxamide;
5-(4-(cyclohexanecarboxamido)cyclohexylamino)-3-(3-methylisothiazol-5-ylamino)pyrazine-2-
carboxamide;
5-(4-benzamidocyclohexylamino)-3-(3-methylisothiazol-5-ylamino)pyrazine-2-carboxamide
3-(3-methylisothiazol-5-ylamino)-5-(4-(nicotinamido)cyclohexylamino)pyrazine-2-
carboxamide;
N-(4-(5-carbamoyl-6-(3-methylisothiazol-5-ylamino)pyrazin-2-ylamino)cyclohexyl)isoxazole-
5-carboxamide;
N-(4-(5-carbamoyl-6-(3-methylisothizol-5-ylamino)pyrazin-2-ylamino)cyclohexyl)thiazole-2-
carboxamide;
3-(3-methylisothiazol-5-ylamino)-5-(4-(tetrahydro-2H-pyran-4-
carboxamido)cyclohexylamino)pyrazine-2-carboxamide;
3-(4-(4-(dimethylamino)benzamido)cyclohexylamino)-5-(4-(morpholine-4-
carbonyl)phenylamino)-1,2,4-triazine-6-carboxamide;
3-(4-(4-isopropylbenzamido)cyclohexylamino)-5-(4-(morpholine-4-carbonyl)phenylamino)-
1,2,4-triazine-6-carboxamide;
3-(4-(morpholine-4-carbonyl)phenylamino)-5-(4-(4-
(trifluoromethyl)benzamido)cyclohexylamino)pyrazine-2-carboxamide;
5-(4-(4-chlorobenzamido)cyclohexylamino)-3-(4-(morpholine-4-
carbonyl)phenylamino)pyrazine-2-carboxamide;
3-(4-(morpholine-4-carbonyl)phenylamino)-5-(4-(picolinamido)cyclohexylamino)pyrazine-2-
carboxamide;
N-(4-(5-carbamoyl-6-(4-(morpholine-4-carbonyl)phenylamino)pyrazin-2-
ylamino)cyclohexyl)thiazole-2-carboxamide;
N-(4-(5-carbamoyl-6-(4-(morpholine-4-carbonyl)phenylamino)pyrazin-2-
ylamino)cyclohexyl)benzo[d]thiazole-2-carboxamide;
5-(4-(cycloheptanecarboxamido)cyclohexylamino)-3-(4-(morpholine-4-
carbonyl)phenylamino)pyrazine-2-carboxamide; and
3-(4-(morpholine-4-carbonyl)phenylamino)-5-(4-(tetrahydro-2H-pyran-4-
carboxamido)cyclohexylamino)pyrazine-2-carboxamide;

or a pharmaceutically acceptable solvate, pharmaceutically acceptable salt, or pharmaceutically acceptable prodrug thereof.

In one aspect, provided herein is a compound of Formula (C-IC) having the structure:

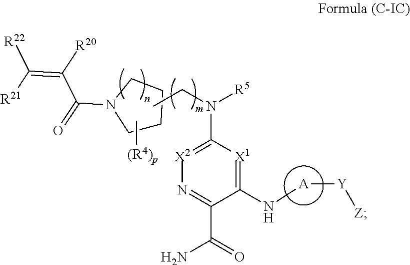

wherein:
A, X¹, X², Y, Z, R⁴, R⁵, n and p are as defined herein;
R²⁰, R²¹ and R²² are each independently H, CN, halo, substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, or pharmaceutically acceptable prodrug thereof.

In some embodiments, R²⁰ and R²¹ are H, R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, all of R²⁰, R²¹ and R²² are H. In some embodiments, R²⁰ and R²¹ together form a bond and R²² is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl. In some embodiments, R²⁰ is CN. In some embodiments, R²⁰ is halo, such as F.

In some embodiments, R²⁰, R²¹ and R²² are independently H, F, Cl, C₁-C₄ alkyl or cycloalkyl, CF₃, or CN. In some embodiments, one of R²⁰ and R²¹ is H, the other one of R²⁰ and R²¹ is F, Cl, C₁-C₄ alkyl, C₃-C₈ cycloalkyl, CF₃, or CN, and R²² is H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl.

In another embodiment are compounds having the structure of any one of Formulas (C-IIa)-(C-IIe):

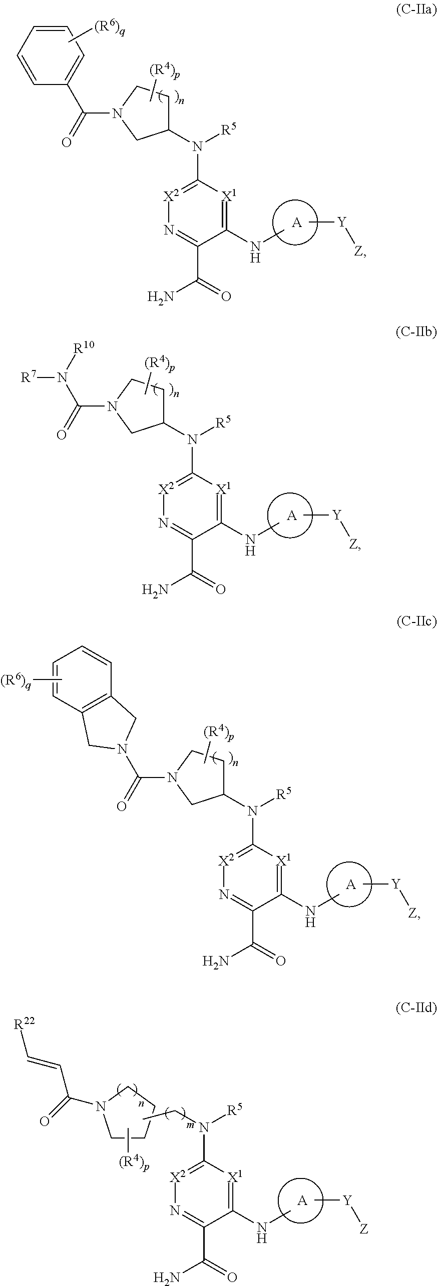

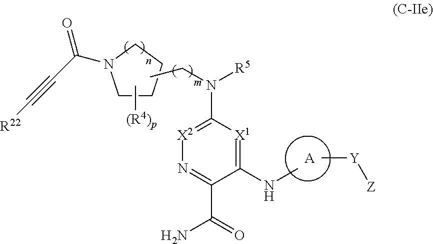

wherein:
A, X¹, X², Y, Z, R⁴, R⁵, R⁷, R¹⁰, R²², m, n and pare as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In some embodiments, the compound of the invention is not (R)-3-(5-carbamoyl-6-(3-methylisothiazol-5-ylamino)pyrazin-2-ylamino)-N,N-dimethylazepane-1-carboxamide or a pharmaceutically acceptable solvate, pharmaceutically acceptable salt, or pharmaceutically acceptable prodrug thereof.

In some embodiments, the compounds of the invention have the structure of Formula (C-IIIa), (C-IIIb) or (C-IIIc):

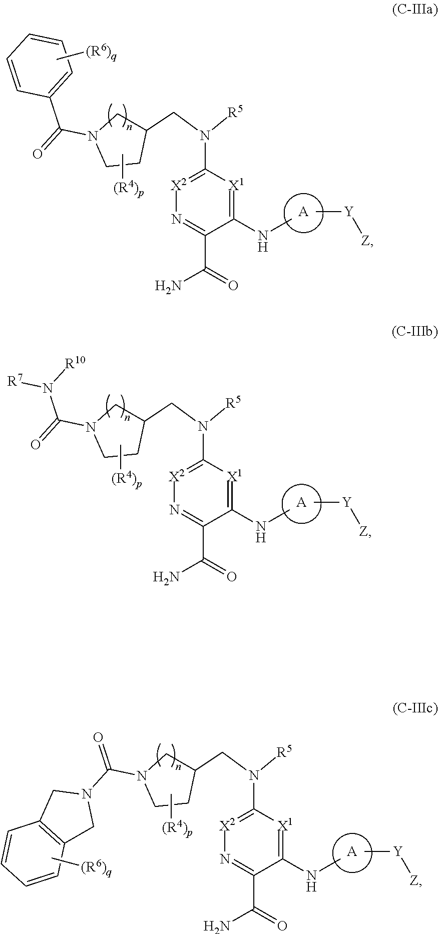

wherein:
A, X¹, X², Y, Z, R⁴, R⁵, R⁷, R¹⁰, n and p are as defined herein;
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; R³ is as defined herein; and
q is 0, 1, 2 or 3;
or a pharmaceutically acceptable solvate, pharmaceutically acceptable salt, or pharmaceutically acceptable prodrug thereof.

In one aspect, provided herein is a compound of Formula (C-VIII) having the structure:

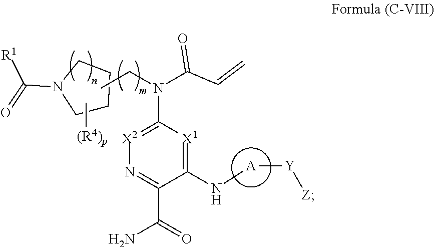

or a pharmaceutically acceptable solvate, pharmaceutically acceptable salt, or pharmaceutically acceptable prodrug thereof, the variables are as defined herein.

In some embodiments the present invention provides a compound of Formula (C-I), (C-IA)-(C-IC), (C-IIa)-(C-IIe), (C-IIIa)-(C-IIIc) or (C-VIII) wherein ring A is substituted or unsubstituted C₆-C₁₂aryl. In some embodiments the compound is a compound of Formula (C-I), (C-IA)-(C-IC), (C-IIa)-(C-IIe), (C-IIIa)-(C-IIIc) or (C-VIII) wherein ring A is phenyl. In some embodiments the compound is a compound of Formula (C-I), (C-IA)-(C-IC), (C-IIa)-(C-IIe), (C-IIIa)-(C-IIIc) or (C-VIII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments the compound is a compound of Formula (C-(I), (C-IA)-(C-IC), (C-IIa)-(C-IIe), (C-IIIa)-(C-IIIc) or (C-VIII) wherein Y is a single bond, —CH₂O—, —OCH₂—, —O—, —N(R³)—, —N(R³)C(O)—, —C(O)N(R³)—, or substituted or unsubstituted C₁-C₄alkylene. In some embodiments the compound is a compound of Formula (C-I), (C-IA)-(C-IC), (C-IIa)-(C-IIe), (C-IIIa)-(C-IIIc) or (C-VII) wherein Y is a single bond, —C(O)—, or —C(O)N(R³)—. In some embodiments the compound is a compound of Formula (C-I), (C-IA)-(C-IC), (C-IIa)-(C-IIe), (C-IIIa)-(C-IIIc) or (C-VIII) wherein Z is substituted or unsubstituted C₁-C₃alkyl. In some embodiments the compound is a compound of Formula (C-I), (C-IA)-(C-IC), (C-IIa)-(C-IIe), (C-IIIa)-(C-IIIc) or (C-VIII) wherein Z is Me, Et, or i-Pr. In some embodiments the compound is a compound of Formula (C-I), (C-IA)-(C-IC), (C-IIa)-(C-IIe), (C-IIIa)-(C-IIIc) or (C-VIII) wherein Z is substituted or unsubstituted C₂-C₇heterocycloalkyl, substituted or unsubstituted C₆-C₁₂aryl, or substituted or unsubstituted C₁-C₁₂heteroaryl.

In some embodiments the present invention provides a compound of Formula (C-I), (C-IA)-(C-IC), (C-IIa)-(C-IIe), (C-IIIa)-(C-IIIc) or (C-VIII) wherein ring A is substituted or unsubstituted C₁-C₁₂heteroaryl. In some embodiments the compound is a compound of Formula (C-I), (C-IA)-(C-IC), (C-IIa)-(C-IIe), (C-IIIa)-(C-IIIc) or (C-VIII) wherein ring A is pyridyl. In some embodiments the compound is a compound of Formula (C-I), (C-IA)-(C-IC), (C-IIa)-(C-IIe), (C-IIIa)-(C-IIIc) or (C-VIII) wherein A is isothiazolyl. In some embodiments the compound is a compound of Formula (C-I), (C-IA)-(C-IC), (C-IIa)-(C-IIe), (C-IIIa)-(C-IIIc) or (C-VIII) wherein Y is a single bond, —CH₂O—, —OCH₂—, —O—, —N(R³)—, —C(O)—, —N(R³)C(O)—, —C(O)N(R³)—, or substituted or unsubstituted C₁-C₄alkylene. In some embodiments the compound is a compound of Formula (C-I), (C-IA)-(C-IC), (C-IIa)-(C-IIe), (C-IIIa)-(C-IIIc) or (C-VIII) wherein Y is a single bond, —C(O)—, or —C(O)N(R³)—. In some embodiments the compound is a compound of Formula (C-I), (C-IA)-(C-IC), (C-IIa)-(C-IIe), (C-IIIa)-(C-IIIc) or (C-VIII) wherein Z is substituted or unsubstituted C₁-C₃alkyl. In some embodiments the compound is a compound of Formula (C-I), (C-IA)-(C-IC), (C-IIa)-(C-IIe), (C-IIIa)-(C-IIIc) or (C-VIII) wherein Z is Me, Et, or i-Pr. In some embodiments the compound is a compound of Formula (C-I), (C-IA)-(C-IC), (C-IIa)-(C-IIe), (C-IIIa)-(C-IIIc) or (C-VIII) wherein Z is substituted or unsubstituted C₂-C₇heterocycloalkyl, substituted or unsubstituted C₆-C₁₂aryl, or substituted or unsubstituted C₁-C₁₂heteroaryl. In some embodiments the compound is a compound of Formula (C-I), (C-IA)-(C-IC), (C-IIa)-(C-IIe), (C-IIIa)-(C-IIc) or (C-VIII) wherein A is isothiazolyl; Y is a single bond; and Z is Me.

In some embodiments, the present invention provides a compound of Formula (A-I), (A-II), (A-VI), (A-IA)-(A-IH), (A-IIa), (A-IIb), (A-IIIa), (A-IIIb) or (A-VII) wherein A is substituted or unsubstituted C₁-C₁₂heteroaryl. In some embodiments, the present invention provides a compound of Formula (A-I), (A-II), (A-VI), (A-IA)-(A-IH), (A-IIa), (A-IIb), (A-IIa), (A-IIIb) or (A-VII) wherein Y is a single bond; in some embodiments, the present invention provides a compound of Formula (A-I), (A-II), (A-VI), (A-IA)-(A-IH), (A-IIa), (A-IIb), (A-IIIa), (A-IIIb) or (A-VII) wherein Z is H, substituted or unsubstituted C₁-C₃alkyl, substituted or unsubstituted C₃-C₆cycloalkyl, substituted or unsubstituted C₆-C₁₂aryl, or substituted or unsubstituted C₁-C₁₂heteroaryl.

In some embodiments the present invention provides is a compound of Formula (C-I), (C-IA)-(C-IC), (C-IIa)-(C-IIe), (C-IIIa)-(C-IIIc) or (C-VIII) wherein A-Y—Z is other than

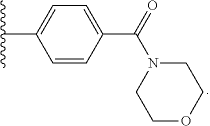 [SMILES: *c1ccc(C(=O)N2CCOCC2)cc1]

In another aspect, provided herein is a compound of Formula (C-IVa), (C-IVb), (C-IVc), (C-IVd), (C-Va), (C-Vb), (C-Vc), (C-Vd), (C-VIa), (C-VIb), (C-VIc), (C-VId), (C-VIIa), (C-VIIb), (C-VIIc) or (C-VIId) having the structure:

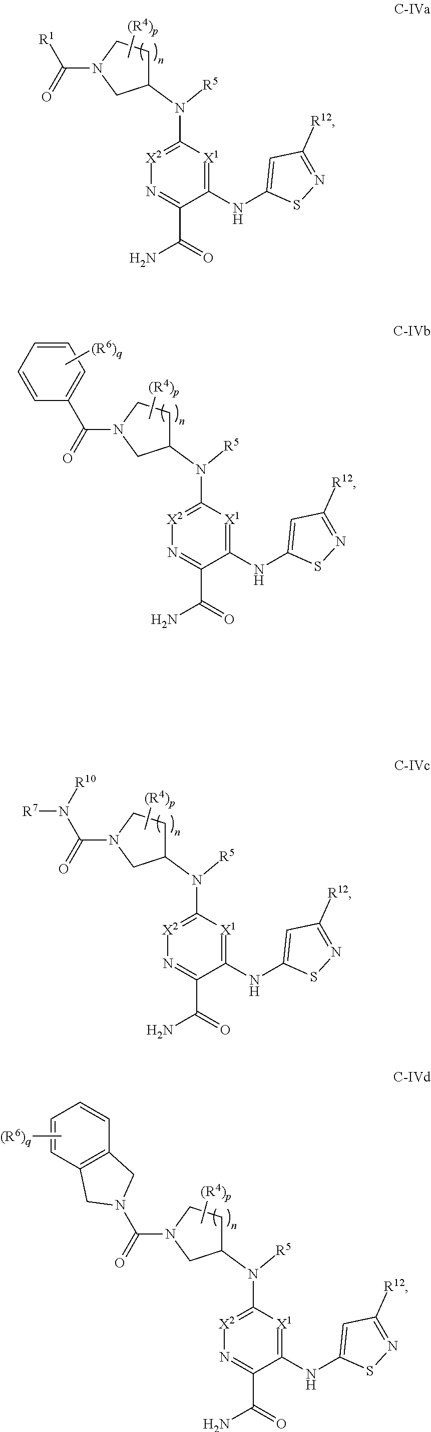

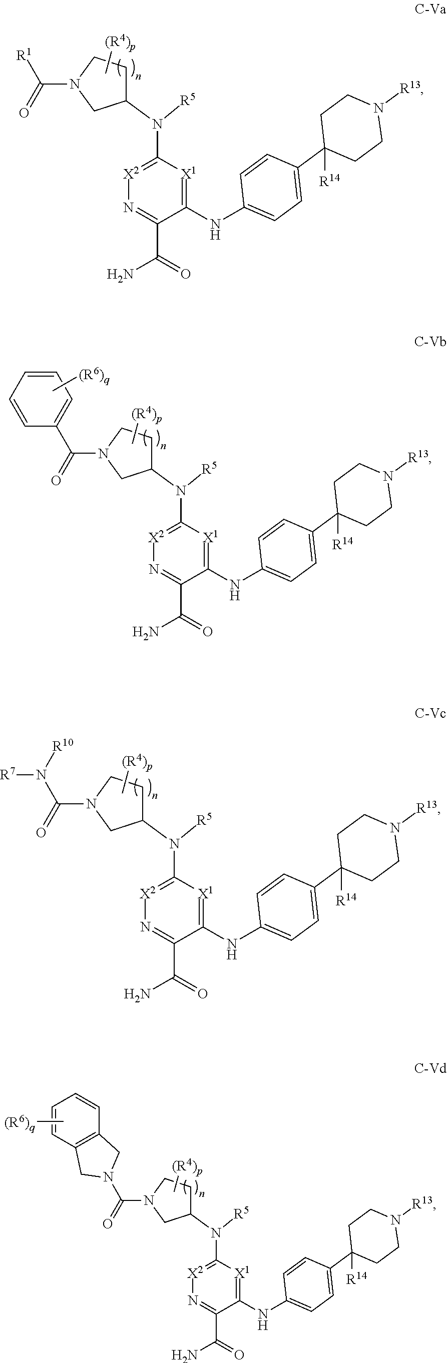

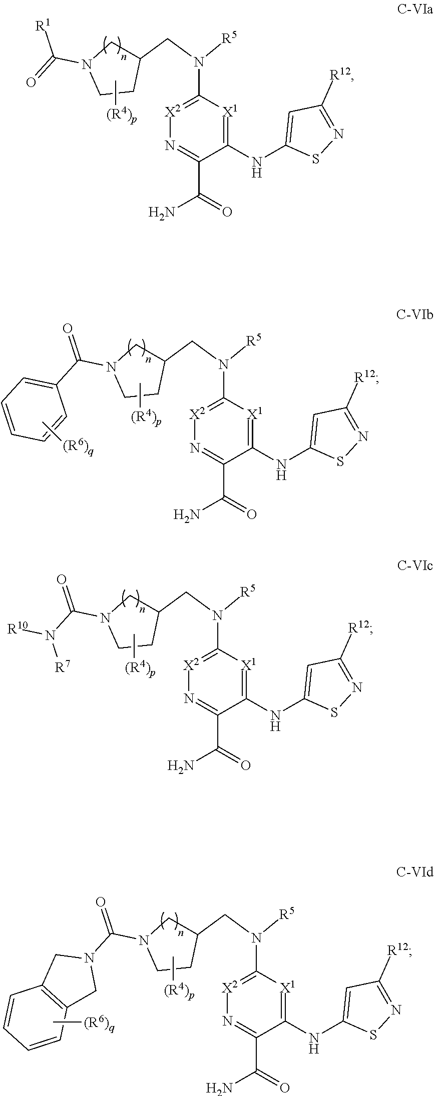

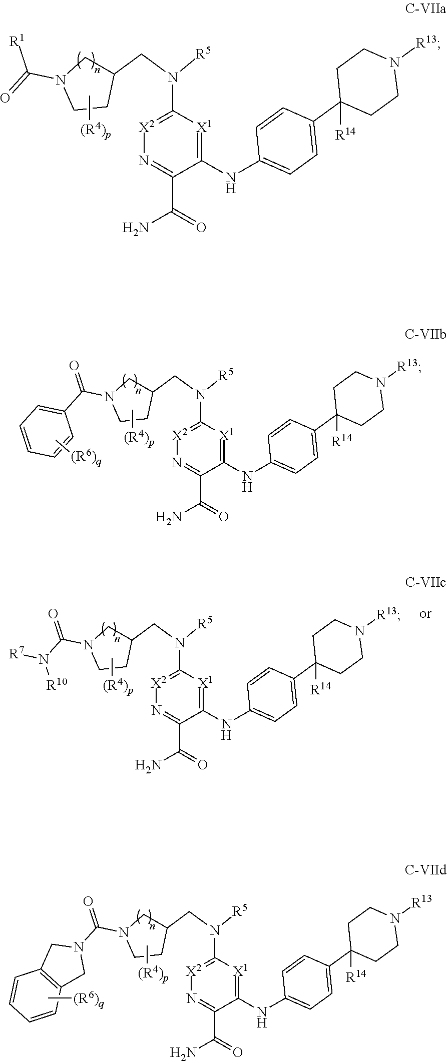

wherein:
X¹, X², R¹, R⁴, R⁵, R⁶, R⁷, R¹⁰, n, p and q are as defined herein;
R¹² is substituted or unsubstituted C₁-C₃alkyl,
R¹³ is substituted or unsubstituted C₃-C₇cycloalkyl, and
R¹⁴ is hydrogen or unsubstituted C₁-C₃alkyl,
or a pharmaceutically acceptable solvate, pharmaceutically acceptable salt, or pharmaceutically acceptable prodrug thereof.

In some embodiments, R¹² is unsubstituted C₁-C₃alkyl, such as methyl or ethyl.

In some embodiments, R¹³ is unsubstituted C₃-C₇alkyl, such as cyclopropyl, cyclobutyl, cyclopentyl, cyclohexyl or cycloheptyl.

In further embodiments of the aforementioned embodiments the invention provides a compound of Formula (C-I), (C-IA)-(C-IC), (C-IIa)-(C-IIe), (C-IIIa)-(C-IIIc), (IVa)-(C-IVd), (C-Va)-(C-Vd), (C-VIa)-(C-VId), (C-VIIa)-(C-VIId) or (C-VIII) wherein X¹ and X² are both N. In further embodiments of the aforementioned embodiments the invention provides a compound of Formula (C-I), (C-IA)-(C-IC), (C-IIa)-(C-IIe), (C-IIIa)-(C-IIIc), (IVa)-(C-IVd), (C-Va)-(C-Vd), (C-VIa)-(C-VId), (C-VIIa)-(C-VIId) or (C-VIII) wherein X¹ and X² are independently C(R²). In further embodiments of the aforementioned embodiments the invention provides a compound of Formula (C-I), (C-IA)-(C-IC), (C-IIa)-(C-IIe), (C-IIIa)-(C-IIIc), (IVa)-(C-IVd), (C-Va)-(C-Vd), (C-VIa)-(C-VId), (C-VIIa)-(C-VIId) or (C-VIII) wherein X¹ is N and X² is C(R²). In some embodiments, each R² is independently H, substituted or unsubstituted C₁-C₄alkyl, —CN, or halogen. In some embodiments, R² is H. In some embodiments, X¹ and X² are both N or are both CH; or X¹ is N and X² is CH.

In some embodiments the invention provides a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId) wherein q is 0. In some embodiments the compound is a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId) wherein q is 1. In some embodiments the compound is a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId) wherein q is 2 or 3.

In some embodiments the invention provides a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId), wherein R⁶ is independently Me, Et, i-Pr, cyclopropyl, cyclobutyl, cyclopentyl, Cl, F, amino, or dimethylamino. In some embodiments the compound is a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId), wherein at least one R⁶ is independently F, CF₃, OCH₃, OCF₃. In some embodiments the compound is a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId), wherein R⁶ is independently F, CF₃, OCH₃, OCF₃. In some embodiments the compound is a compound of Formula (C-IIa), (C-IIc), (C-IIIa), (C-IIIc), (C-IVb), (C-IVd), (C-Vb), (C-Vd), (C-VIb), (C-VId), (C-VIIb) or (C-VIId) wherein at least one R⁶ is independently —N(R³)₂, or —OH. In some embodiments, at least one R⁶ is —N(CH₃)₂. In some embodiments, at least one R⁶ is —NH₂. In some embodiments, at least one R⁶ is —N(R³)₂. In some embodiments, at least one R⁶ is —OH.

In some embodiments, A, Y, Z, R², R⁴, R⁵, R⁷, R¹⁰, m, n and p are as defined herein; X¹ is N and X² is C(R²); and R¹ is substituted or unsubstituted C₁-C₄alkyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN.

In some embodiments, A, Y, Z, R⁴, R⁵, R⁷, R¹⁰, m, n and p are as defined herein; each of X¹ and X² is N; and R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN.

In some embodiments, A, Y, Z, R⁴, R⁵, R⁷, R¹⁰, m, n and p are as defined herein; X¹ is N and X² is C(H); and R¹ is substituted or unsubstituted phenyl. In some embodiments, the substitution on phenyl is dimethylamino.

In some embodiments, A, Y, Z, R⁴, R⁵, R⁷, R¹⁰, m, n and p are as defined herein; X¹ is N, and X² is C(H); and R¹ is dialkylamino, aminomethyl, or aminopropyl.

In some embodiments, A, Y, Z, R⁴, R⁵, R⁷, R¹⁰, m, n and p are as defined herein; X¹ is N, and X² is N; and R¹ is substituted or unsubstituted C₂-C₄alkenyl. In one embodiment, R¹ is unsubstituted or substituted ethenyl. In one embodiment, R¹ is unsubstituted ethenyl.

In some embodiments, p is 1 and R⁴ is Me.

In some embodiments, R⁵ is H. In some embodiments, R⁵ is Me. In some embodiments, R⁵ is CO—(C₂-C₄alkenyl), such as CO—CH═CH₂.

In more particular embodiments, the group -A-Y—Z is 3-methyl-5-isothiazolyl or 3-phenyl-5-isothiazolyl. In other particular embodiments, the group -A-Y—Z is 4-isopropylmethylphenyl.

In some embodiments, the compound is:

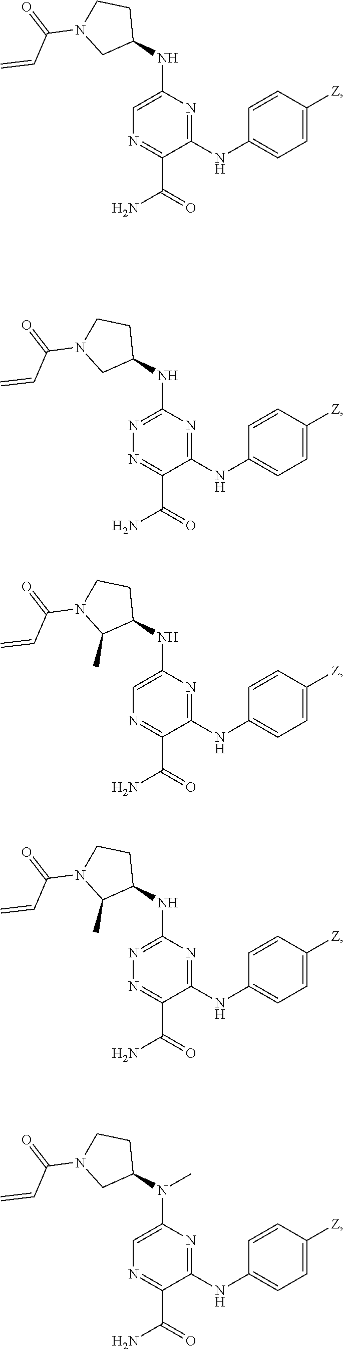

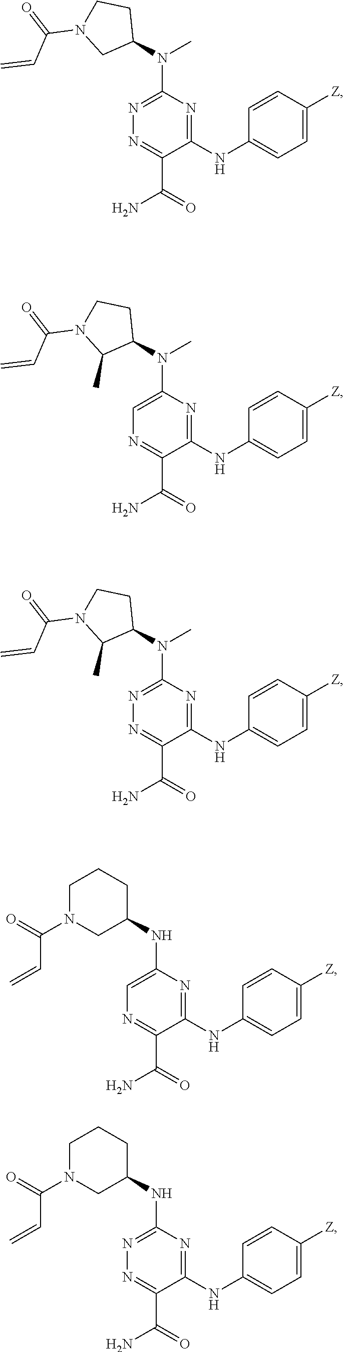

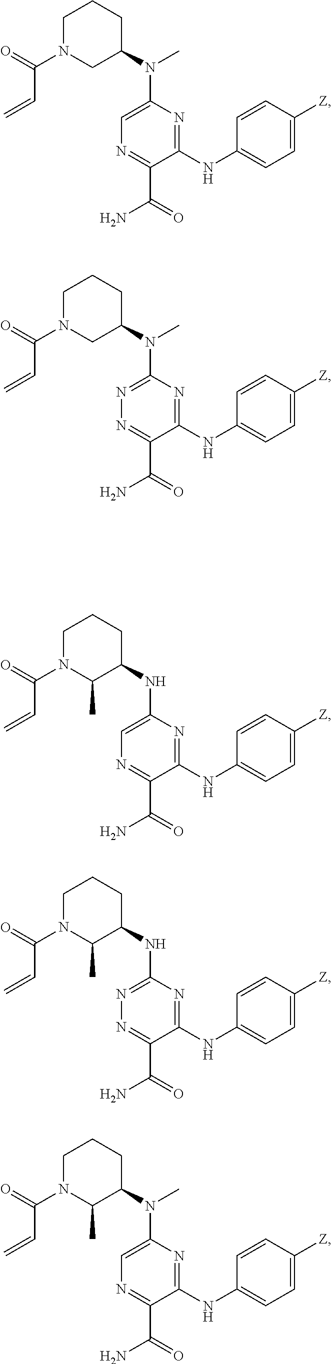

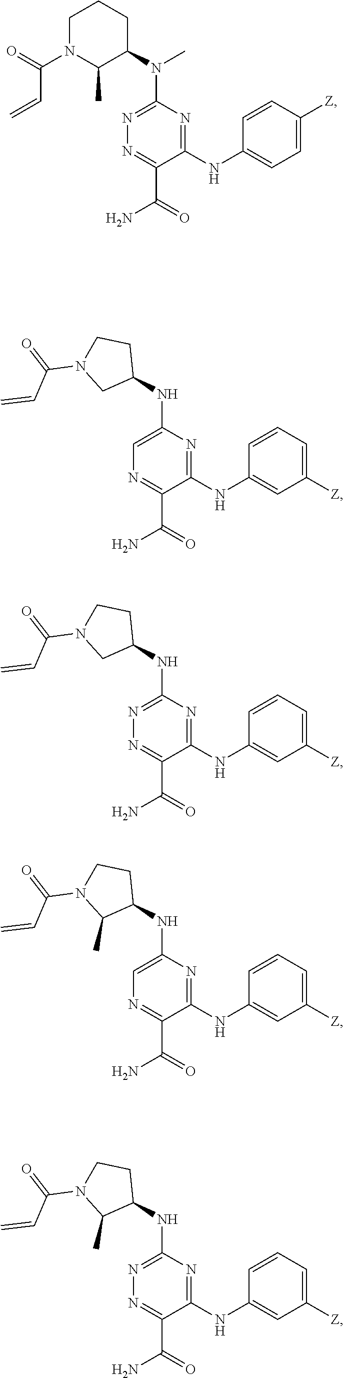

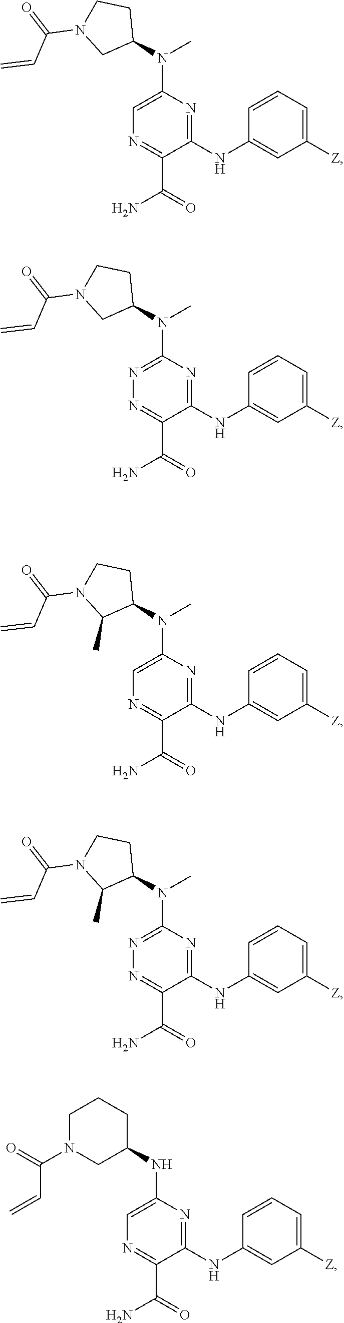

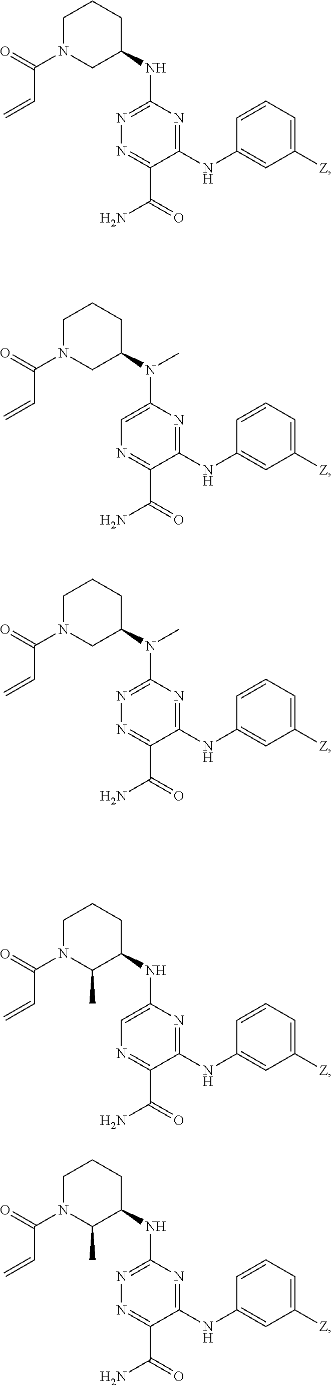

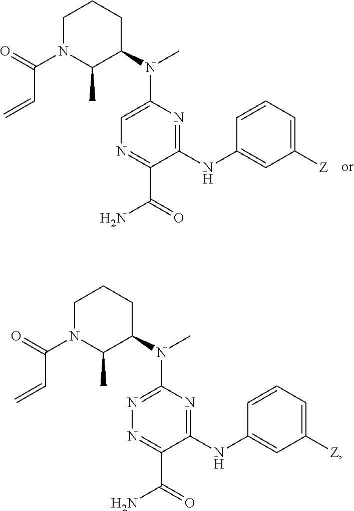

wherein Z is methyl substituted with mono or dialkyl amino, or Z is a 5- or 6-membered heteroaryl optionally substituted with methyl, ethyl, CO—CH═CH₂ or hydroxyl,
or a pharmaceutically acceptable solvate, pharmaceutically acceptable salt, and/or pharmaceutically acceptable prodrug thereof.

In some embodiments, Z is

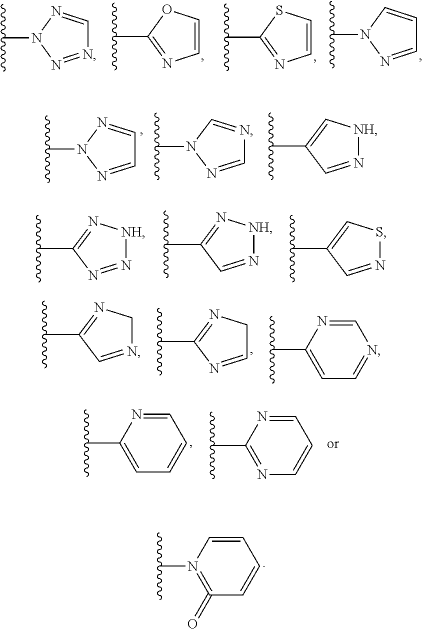

In some embodiments, the compound is selected from:

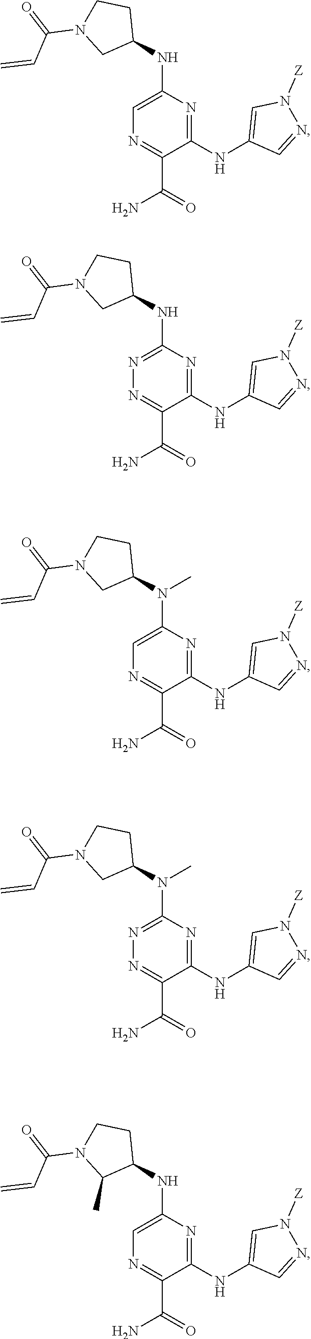

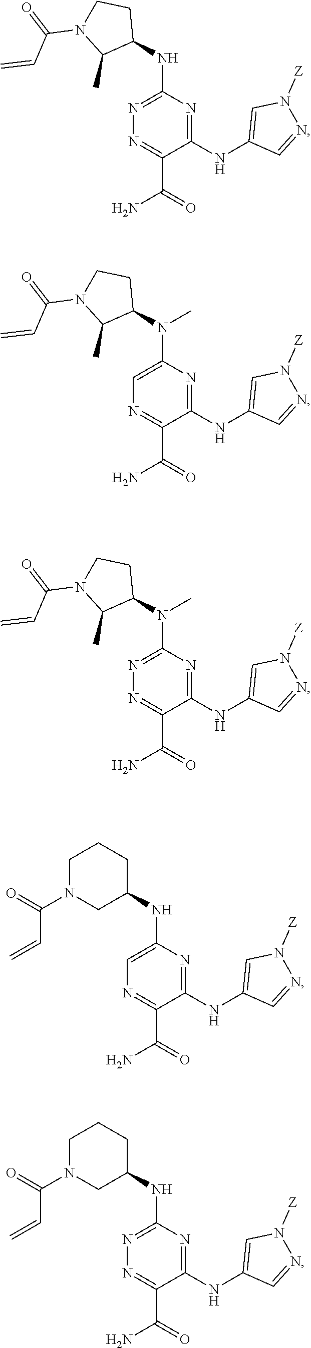

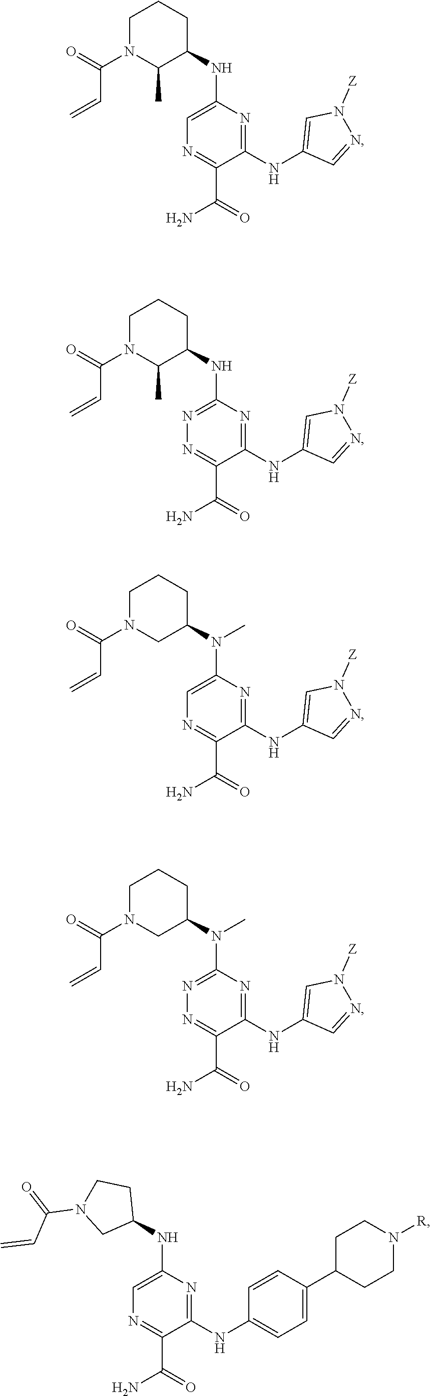

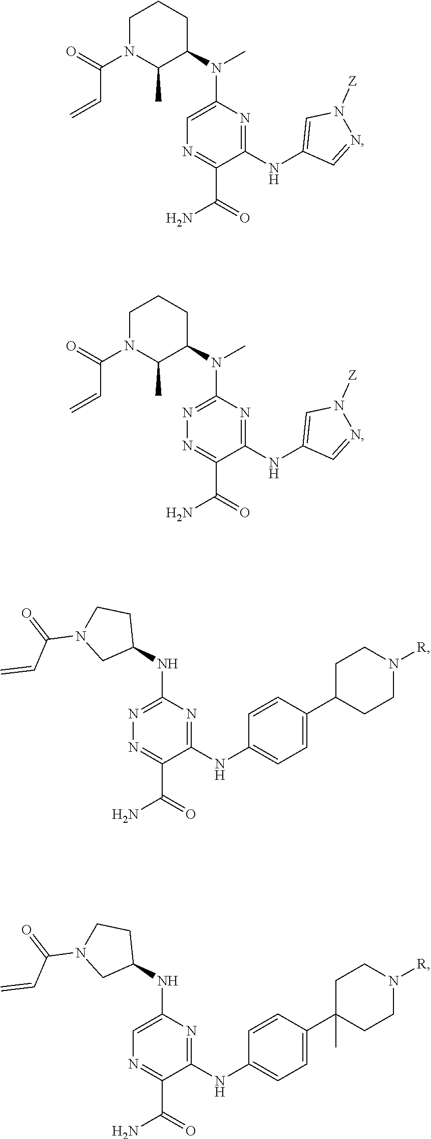

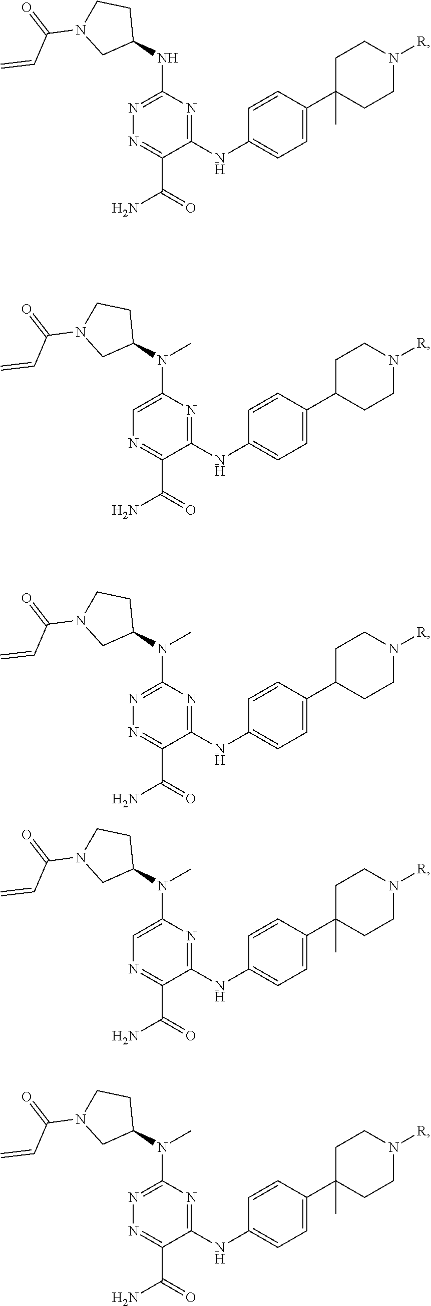

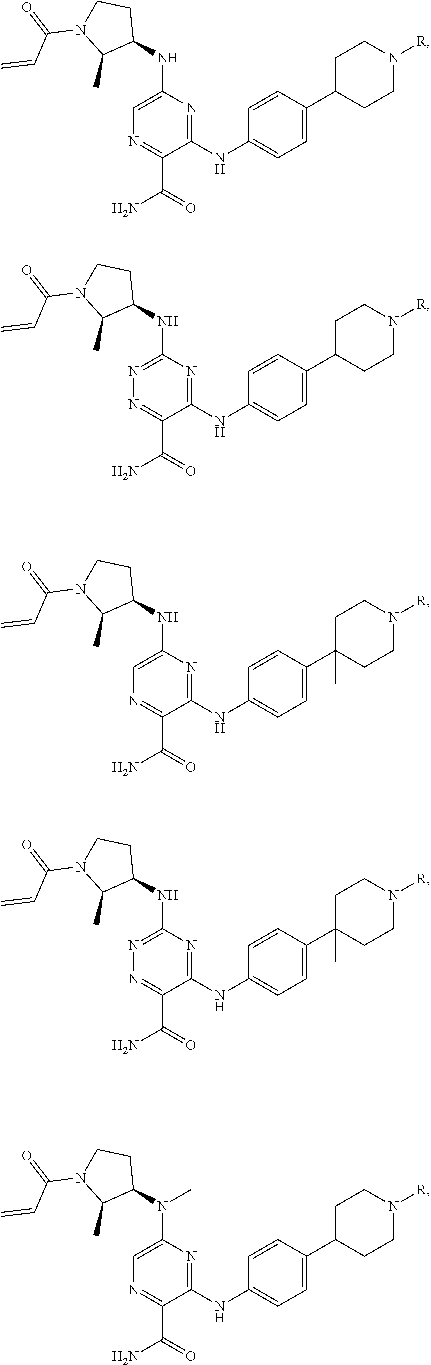

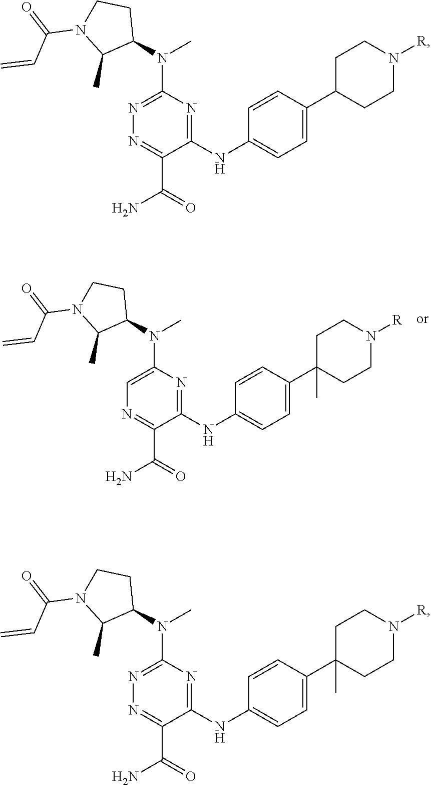

wherein Z and R are methyl, ethyl, isopropyl, cyclopropyl, cyclobutyl, or cyclopentyl, or a pharmaceutically acceptable solvate, pharmaceutically acceptable salt, and/or pharmaceutically acceptable prodrug thereof.

In some embodiments, the compound is selected from:

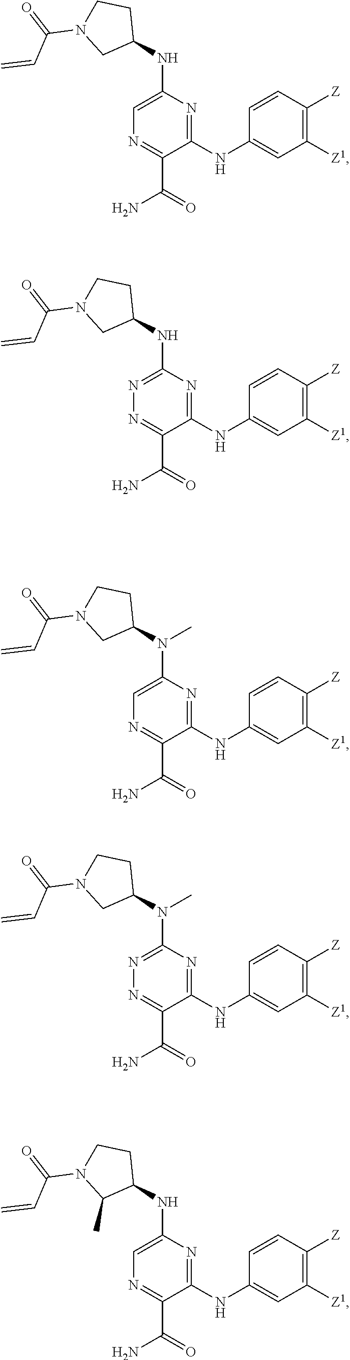

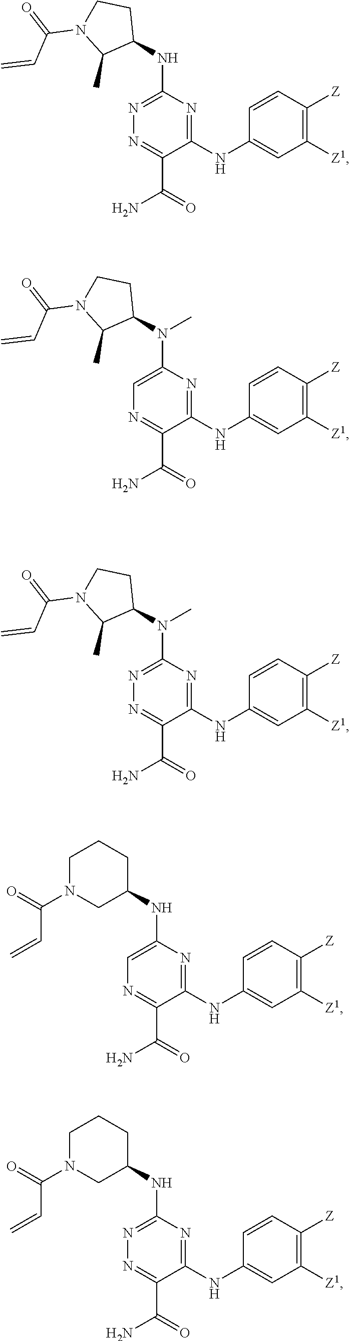

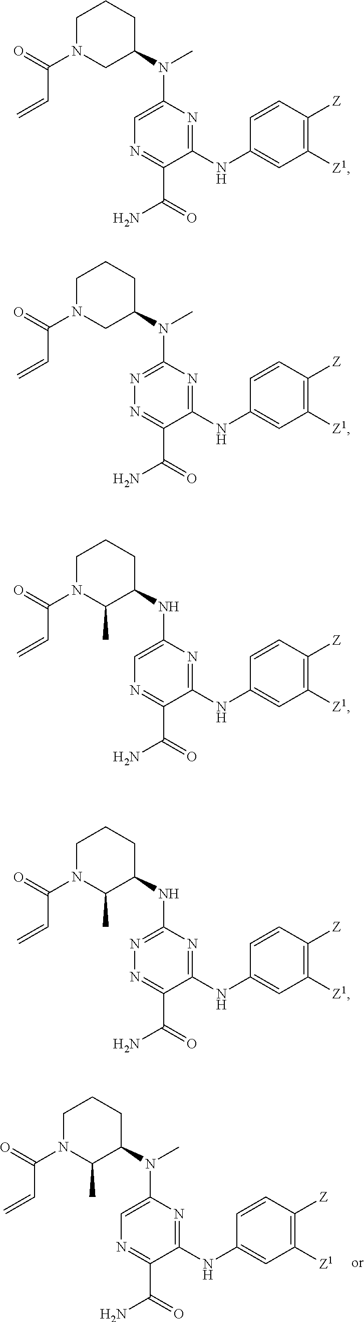

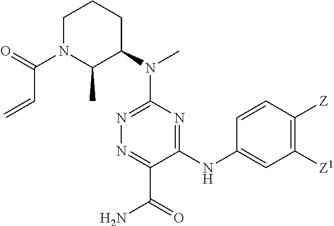

wherein Z and Z¹ are independently H, F, Cl, CN, methyl, ethyl, isopropyl, cyclopropyl, or CF₃, or a pharmaceutically acceptable solvate, pharmaceutically acceptable salt, and/or pharmaceutically acceptable prodrug thereof.

In some embodiments, Z and Z¹ are independently F, Cl, CN, methyl, ethyl, isopropyl, cyclopropyl, or CF₃.

In some embodiments, at least one of Z and Z¹ is other than H.

In some embodiments, the compound is selected from:

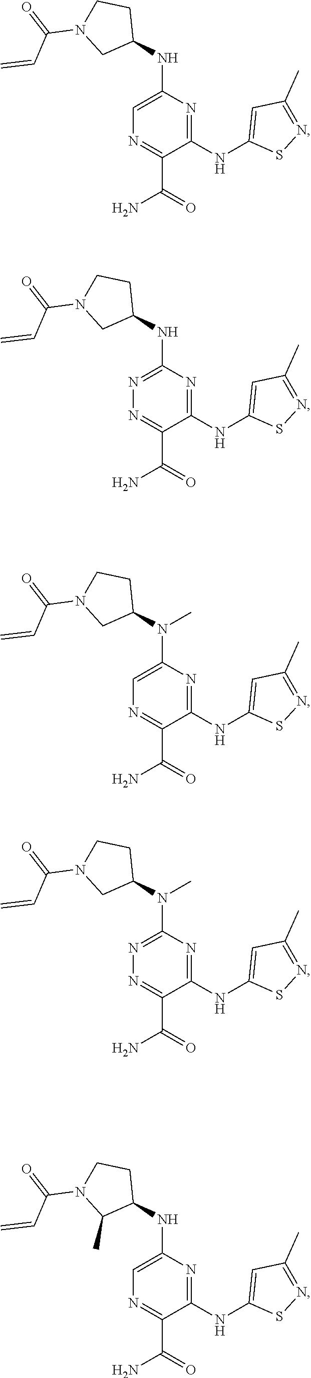 [SMILES: C=CC(=O)N1CC[C@@H](Nc2cnc(C(N)=O)c(Nc3cc(C)ns3)n2)C1 ; C=CC(=O)N1CC[C@@H](Nc2nnc(C(N)=O)c(Nc3cc(C)ns3)n2)C1 ; C=CC(=O)N1CC[C@@H](N(C)c2cnc(C(N)=O)c(Nc3cc(C)ns3)n2)C1 ; C=CC(=O)N1CC[C@@H](N(C)c2nnc(C(N)=O)c(Nc3cc(C)ns3)n2)C1 ; C=CC(=O)N1CC[C@@H](Nc2cnc(C(N)=O)c(Nc3cc(C)ns3)n2)[C@H]1C]

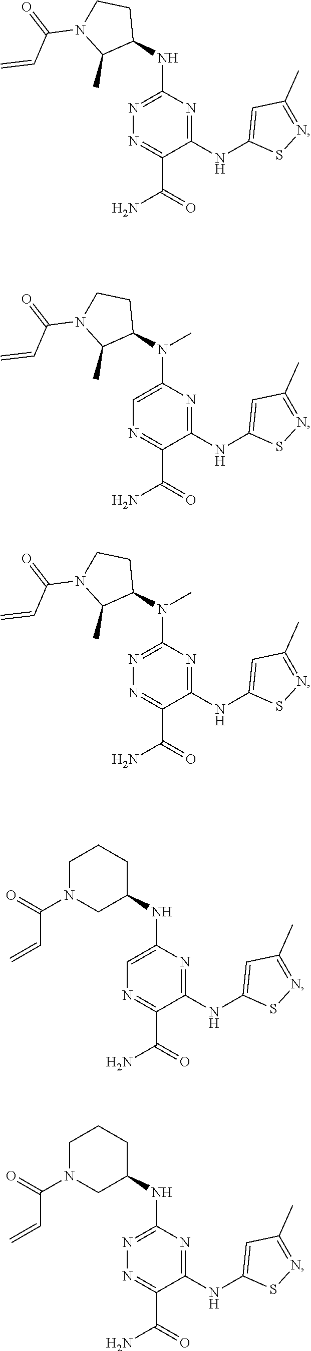 [SMILES: C=CC(=O)N1CC[C@@H](Nc2nnc(C(N)=O)c(Nc3cc(C)ns3)n2)[C@H]1C ; C=CC(=O)N1CC[C@@H](N(C)c2cnc(C(N)=O)c(Nc3cc(C)ns3)n2)[C@H]1C ; C=CC(=O)N1CC[C@@H](N(C)c2nnc(C(N)=O)c(Nc3cc(C)ns3)n2)[C@H]1C ; C=CC(=O)N1CCC[C@@H](Nc2cnc(C(N)=O)c(Nc3cc(C)ns3)n2)C1 ; C=CC(=O)N1CCC[C@@H](Nc2nnc(C(N)=O)c(Nc3cc(C)ns3)n2)C1]

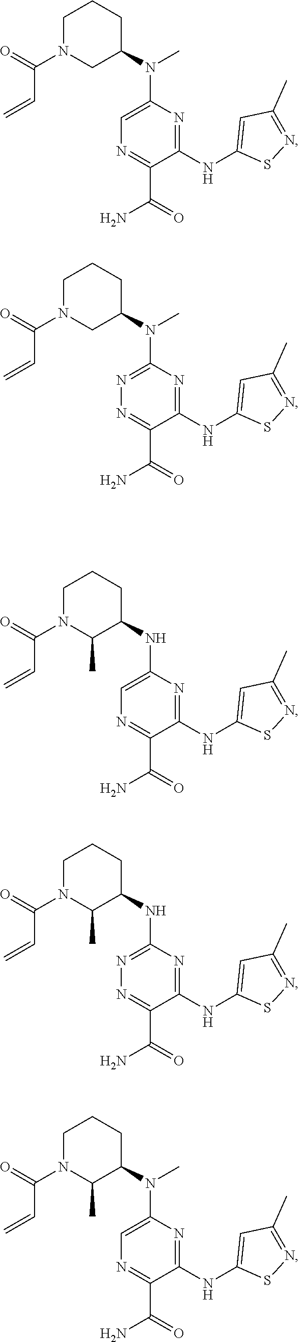 [SMILES: C=CC(=O)N1CCC[C@@H](N(C)c2cnc(C(N)=O)c(Nc3cc(C)ns3)n2)C1 ; C=CC(=O)N1CCC[C@@H](N(C)c2nnc(C(N)=O)c(Nc3cc(C)ns3)n2)C1 ; C=CC(=O)N1CCC[C@@H](Nc2cnc(C(N)=O)c(Nc3cc(C)ns3)n2)[C@H]1C ; C=CC(=O)N1CCC[C@@H](Nc2nnc(C(N)=O)c(Nc3cc(C)ns3)n2)[C@H]1C ; C=CC(=O)N1CCC[C@@H](N(C)c2cnc(C(N)=O)c(Nc3cc(C)ns3)n2)[C@H]1C]

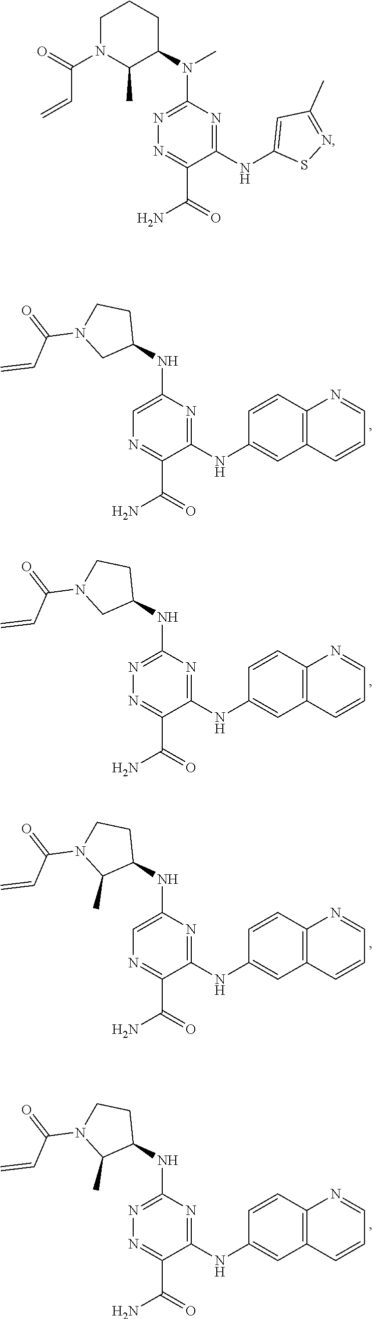 [SMILES: C=CC(=O)N1CCC[C@@H](N(C)c2nnc(C(N)=O)c(Nc3cc(C)ns3)n2)[C@H]1C ; C=CC(=O)N1CC[C@@H](Nc2cnc(C(N)=O)c(Nc3ccc4ncccc4c3)n2)C1 ; C=CC(=O)N1CC[C@@H](Nc2nnc(C(N)=O)c(Nc3ccc4ncccc4c3)n2)C1 ; C=CC(=O)N1CC[C@@H](Nc2cnc(C(N)=O)c(Nc3ccc4ncccc4c3)n2)[C@H]1C ; C=CC(=O)N1CC[C@@H](Nc2nnc(C(N)=O)c(Nc3ccc4ncccc4c3)n2)[C@H]1C]

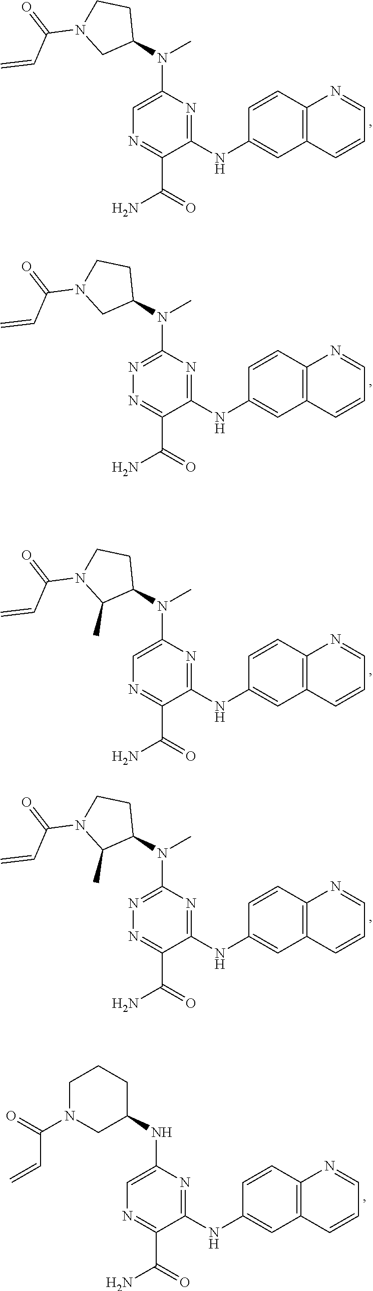 [SMILES: C=CC(=O)N1CC[C@@H](N(C)c2cnc(C(N)=O)c(Nc3ccc4ncccc4c3)n2)C1 ; C=CC(=O)N1CC[C@@H](N(C)c2nnc(C(N)=O)c(Nc3ccc4ncccc4c3)n2)C1 ; C=CC(=O)N1CC[C@@H](N(C)c2cnc(C(N)=O)c(Nc3ccc4ncccc4c3)n2)[C@H]1C ; C=CC(=O)N1CC[C@@H](N(C)c2nnc(C(N)=O)c(Nc3ccc4ncccc4c3)n2)[C@H]1C ; C=CC(=O)N1CCC[C@@H](Nc2cnc(C(N)=O)c(Nc3ccc4ncccc4c3)n2)C1]

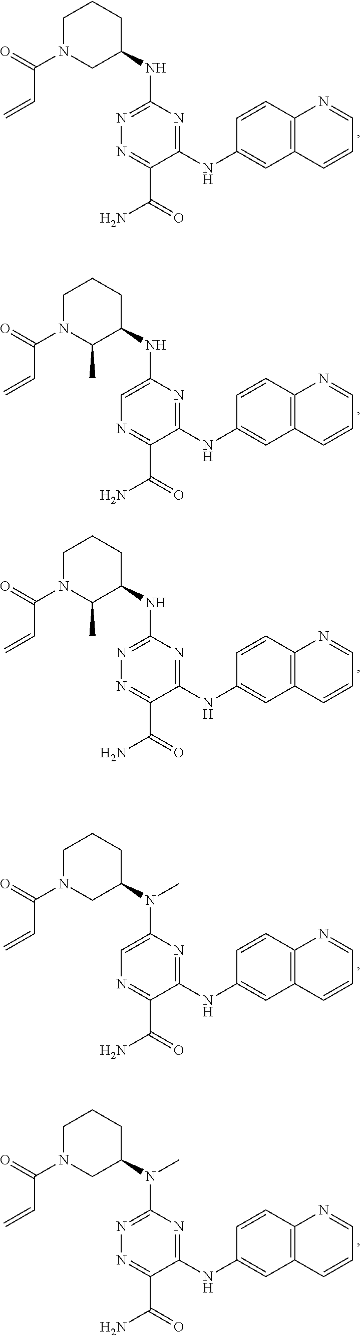 [SMILES: C=CC(=O)N1CCC[C@@H](Nc2nnc(C(N)=O)c(Nc3ccc4ncccc4c3)n2)C1 ; C=CC(=O)N1CCC[C@@H](Nc2cnc(C(N)=O)c(Nc3ccc4ncccc4c3)n2)[C@H]1C ; C=CC(=O)N1CCC[C@@H](Nc2nnc(C(N)=O)c(Nc3ccc4ncccc4c3)n2)[C@H]1C ; C=CC(=O)N1CCC[C@@H](N(C)c2cnc(C(N)=O)c(Nc3ccc4ncccc4c3)n2)C1 ; C=CC(=O)N1CCC[C@@H](N(C)c2nnc(C(N)=O)c(Nc3ccc4ncccc4c3)n2)C1]

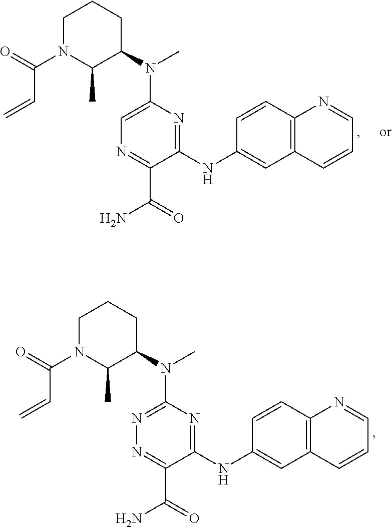 [SMILES: C=CC(=O)N1CCC[C@@H](N(C)c2cnc(C(N)=O)c(Nc3ccc4ncccc4c3)n2)[C@H]1C ; C=CC(=O)N1CCC[C@@H](N(C)c2nnc(C(N)=O)c(Nc3ccc4ncccc4c3)n2)[C@H]1C]

or a pharmaceutically acceptable solvate, pharmaceutically acceptable salt, and/or pharmaceutically acceptable prodrug thereof.

### Formula C-I Compounds

In a particular embodiment, the compound is selected from the group consisting of compounds listed in Table N5:

TABLE N5
Formula C-I Compounds

| Compound Structure | Compound Name |
|---|---|
| 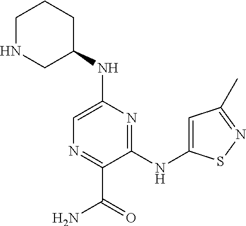 [SMILES: Cc1cc(Nc2nc(N[C@@H]3CCCNC3)cnc2C(N)=O)sn1] | (R)-3-(4-methylisothiazol-5-ylamino)- 5-(piperidin-3-ylamino)pyrazine-2- carboxamide |
| 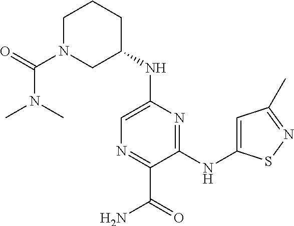 [SMILES: Cc1cc(Nc2nc(N[C@H]3CCCN(C(=O)N(C)C)C3)cnc2C(N)=O)sn1] | (S)-5-(1-(dimethylcarbamoyl)piperidin- 3-ylamino)-3-(3-methylisothiazol-5- ylamino)pyrazine-2-carboxamide |
| 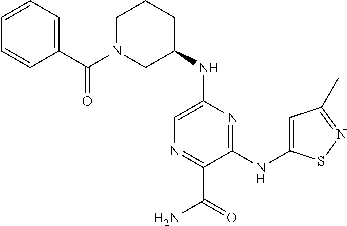 [SMILES: Cc1cc(Nc2nc(N[C@@H]3CCCN(C(=O)c4ccccc4)C3)cnc2C(N)=O)sn1] | (R)-5-(1-benzoylpiperidin-3-ylamino)- 3-(3-methylisothiazol-5- ylamino)pyrazine-2-carboxamide |
| 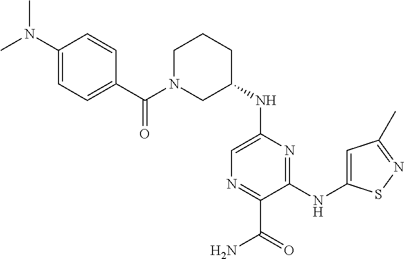 [SMILES: Cc1cc(Nc2nc(N[C@H]3CCCN(C(=O)c4ccc(N(C)C)cc4)C3)cnc2C(N)=O)sn1] | (S)-5-(1-(4- (dimethylamino)benzoyl)piperidin-3- ylamino)-3-(3-methylisothiazol-5- ylamino)pyrazine-2-carboxamide |
| 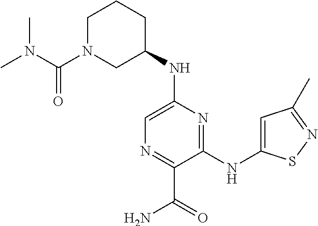 [SMILES: Cc1cc(Nc2nc(N[C@@H]3CCCN(C(=O)N(C)C)C3)cnc2C(N)=O)sn1] | (R)-5-(1- (dimethylcarbamoyl)piperidin-3- ylamino)-3-(3-methylisothiazol-5- ylamino)pyrazine-2-carboxamide |
| 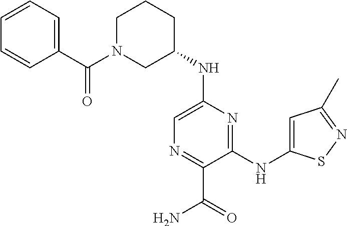 [SMILES: Cc1cc(Nc2nc(N[C@H]3CCCN(C(=O)c4ccccc4)C3)cnc2C(N)=O)sn1] | (S)-5-(1-benzoylpiperidin-3-ylamino)- 3-(3-methylisothiazol-5- ylamino)pyrazine-2-carboxamide |
| 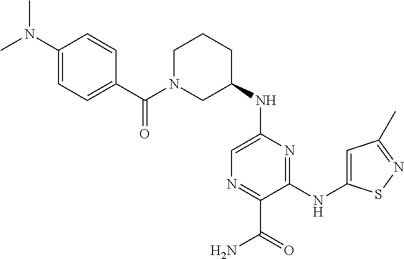 [SMILES: Cc1cc(Nc2nc(N[C@@H]3CCCN(C(=O)c4ccc(N(C)C)cc4)C3)cnc2C(N)=O)sn1] | (R)-5-(1-(4- (dimethylamino)benzoyl)piperidin-3- ylamino)-3-(3-methylisothiazol-5- ylamino)pyrazine-2-carboxamide |
| 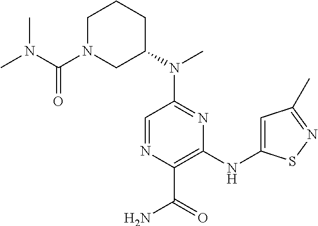 [SMILES: Cc1cc(Nc2nc(N(C)[C@H]3CCCN(C(=O)N(C)C)C3)cnc2C(N)=O)sn1] | (S)-5-((1- (dimethylcarbamoyl)piperidin-3- yl)(methyl)amino)-3-(3- methylisothiazol-5-ylamino)pyrazine- 2-carboxamide |
| 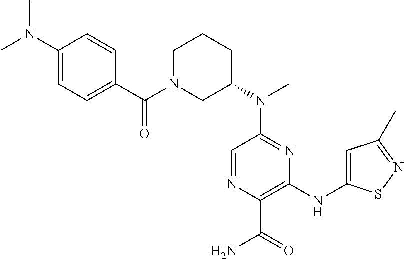 [SMILES: Cc1cc(Nc2nc(N(C)[C@H]3CCCN(C(=O)c4ccc(N(C)C)cc4)C3)cnc2C(N)=O)sn1] | (S)-5-((1-(4- (dimethylamino)benzoyl)piperidin-3- yl)(methyl)amino)-3-(3- methylisothiazol-5-ylamino)pyrazine- 2-carboxamide |
| 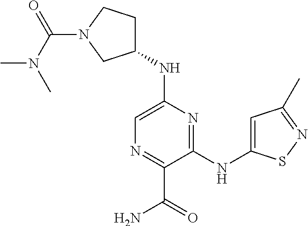 [SMILES: Cc1cc(Nc2nc(N[C@H]3CCN(C(=O)N(C)C)C3)cnc2C(N)=O)sn1] | (S)-5-(1- (dimethylcarbamoyl)pyrrolidin-3- ylamino)-3-(3-methylisothiazol-5- ylamino)pyrazine-2-carboxamide |
| 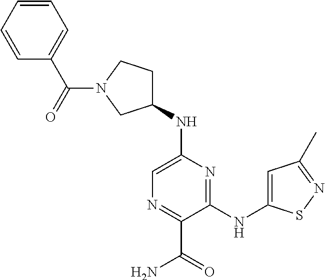 [SMILES: Cc1cc(Nc2nc(N[C@@H]3CCN(C(=O)c4ccccc4)C3)cnc2C(N)=O)sn1] | (R)-5-(1-benzoylpyrrolidin-3-ylamino)- 3-(3-methylisothiazol-5- ylamino)pyrazine-2-carboxamide |
| 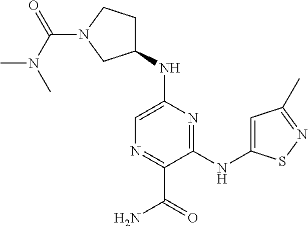 [SMILES: Cc1cc(Nc2nc(N[C@@H]3CCN(C(=O)N(C)C)C3)cnc2C(N)=O)sn1] | (R)-5-(1- (dimethylcarbamoyl)pyrrolidin-3- ylamino)-3-(3-methylisothiazol-5- ylamino)pyrazine-2-carboxamide |
| 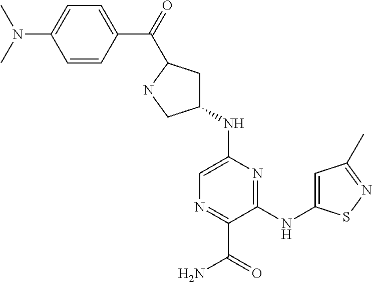 [SMILES: Cc1cc(Nc2nc(N[C@@H]3CNC(C(=O)c4ccc(N(C)C)cc4)C3)cnc2C(N)=O)sn1] | (R)-5-(1-(4- (dimethylamino)benzoyl)pyrrolidin-3- ylamino)-3-(3-methylisothiazol-5- ylamino)pyrazine-2-carboxamide |
| 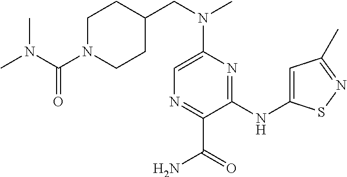 [SMILES: Cc1cc(Nc2nc(N(C)CC3CCN(C(=O)N(C)C)CC3)cnc2C(N)=O)sn1] | 5-(((1-(dimethylcarbamoyl)piperidin-2- yl)methyl)(methyl)amino)-3-(3- methylisothiazol-5-ylamino)pyrazine- 2-carboxamide |
| 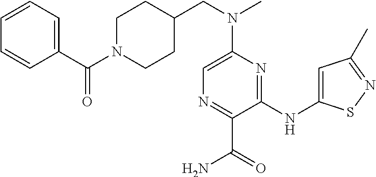 [SMILES: Cc1cc(Nc2nc(N(C)CC3CCN(C(=O)c4ccccc4)CC3)cnc2C(N)=O)sn1] | 5-(((1-benzoylpiperidin-4- yl)methyl)(methyl)amino)-3-(3- methylisothiazol-5-ylamino)pyrazine- 2-carboxamide |
| 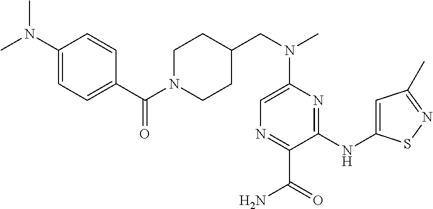 [SMILES: Cc1cc(Nc2nc(N(C)CC3CCN(C(=O)c4ccc(N(C)C)cc4)CC3)cnc2C(N)=O)sn1] | 5-(((1-(4- (dimethylamino)benzoyl)piperidin-4- yl)methyl)(methyl)amino)-3-(3- methylisothiazol-5-ylamino)pyrazine- 2-carboxamide |
| 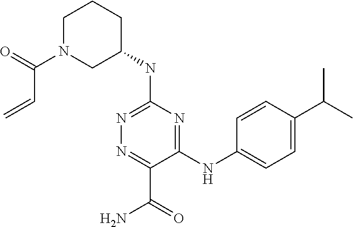 [SMILES: C=CC(=O)N1CCC[C@H](Nc2nnc(C(N)=O)c(Nc3ccc(C(C)C)cc3)n2)C1] | (S)-3-(1-acryloylpiperidin-3-ylamino)- 5-(4-isopropylphenylamino)-1,2,4- triazine-6-carboxamide |
| 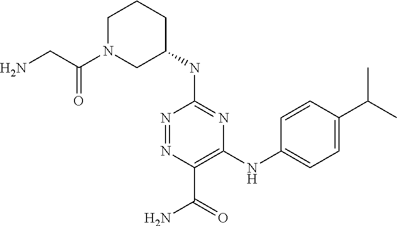 [SMILES: CC(C)c1ccc(Nc2nc(N[C@H]3CCCN(C(=O)CN)C3)nnc2C(N)=O)cc1] | (S)-3-(1-(2-aminoacetyl)piperidin-3- ylamino)-5-(4-isopropylphenylamino)- 1,2,4-triazine-6-carboxamide |
| 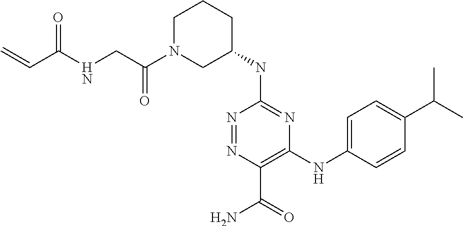 [SMILES: C=CC(=O)NCC(=O)N1CCC[C@H](Nc2nnc(C(N)=O)c(Nc3ccc(C(C)C)cc3)n2)C1] | (S)-3-(1-(2-acrylamidoacetyl)piperidin- 3-ylamino)-5-(4- isopropylphenylamino)-1,2,4-triazine- 6-carboxamide |
| 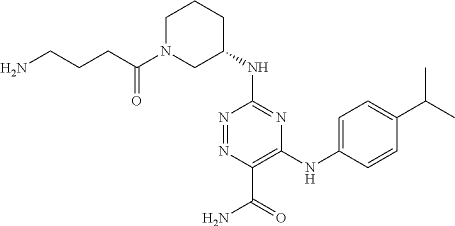 [SMILES: CC(C)c1ccc(Nc2nc(N[C@H]3CCCN(C(=O)CCCN)C3)nnc2C(N)=O)cc1] | Synthesis of (S)-3-(1-(4- aminobutanoyl)piperidin-3-ylamino)-5- (4-isopropylphenylamino)-1,2,4- triazine-6-carboxamide |
| 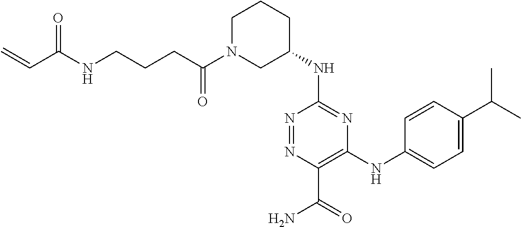 [SMILES: C=CC(=O)NCCCC(=O)N1CCC[C@H](Nc2nnc(C(N)=O)c(Nc3ccc(C(C)C)cc3)n2)C1] | (S)-3-(1-(4- acrylamidobutanoyl)piperidin-3- ylamino)-5-(4-isopropylphenylamino)- 1,2,4-triazine-6-carboxamide |
| 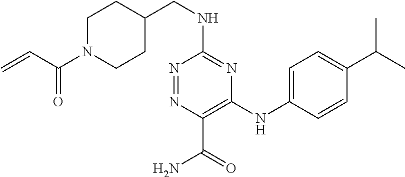 [SMILES: C=CC(=O)N1CCC(CNc2nnc(C(N)=O)c(Nc3ccc(C(C)C)cc3)n2)CC1] | 3-((1-acryloylpiperidin-2- yl)methylamino)-5-(4- isopropylphenylamino)-1,2,4-triazine- 6-carboxamide |
| 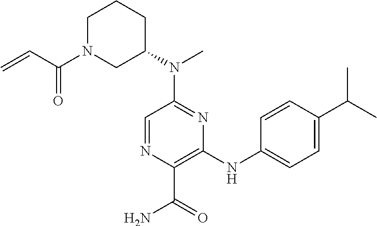 [SMILES: C=CC(=O)N1CCC[C@H](N(C)c2cnc(C(N)=O)c(Nc3ccc(C(C)C)cc3)n2)C1] | (S)-5-((1-acryloylpiperidin-3- yl)(methyl)amino)-3-(4- isopropylphenylamino)pyrazine-2- carboxamide |
| 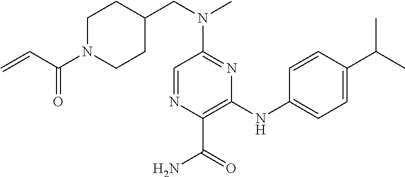 [SMILES: C=CC(=O)N1CCC(CN(C)c2cnc(C(N)=O)c(Nc3ccc(C(C)C)cc3)n2)CC1] | (S)-5-((1-acryloylpiperidin-3- yl)(methyl)amino)-3-(4- isopropylphenylamino)pyrazine-2- carboxamide |
| 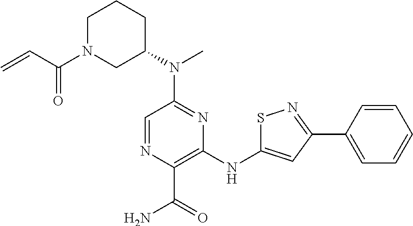 [SMILES: C=CC(=O)N1CCC[C@H](N(C)c2cnc(C(N)=O)c(Nc3cc(-c4ccccc4)ns3)n2)C1] | 5-(((1-acryloylpiperidin-4- yl)methyl)(methyl)amino)-3-(3- phenylisothiazol-5-ylamino)pyrazine- 2-carboxamide |
| 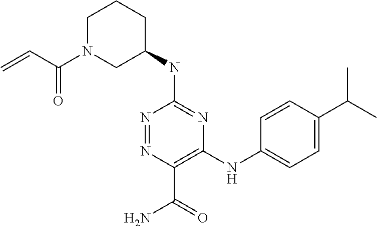 [SMILES: C=CC(=O)N1CCC[C@@H](Nc2nnc(C(N)=O)c(Nc3ccc(C(C)C)cc3)n2)C1] | (R)-3-(1-acryloylpiperidin-3-ylamino)- 5-(4-isopropylphenylamino)-1,2,4- triazine-6-carboxamide |
| 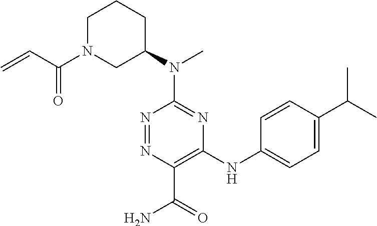 [SMILES: C=CC(=O)N1CCC[C@@H](N(C)c2nnc(C(N)=O)c(Nc3ccc(C(C)C)cc3)n2)C1] | (R)-3-((1-acryloylpiperidin-3- yl)(methyl)amino)-5-(4- isopropylphenylamino)-1,2,4-triazine- 6-carboxamide |
| 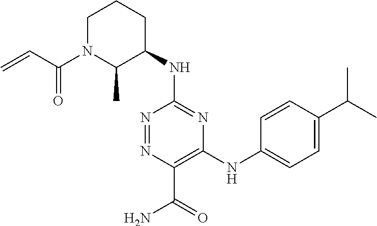 [SMILES: C=CC(=O)N1CCC[C@@H](Nc2nnc(C(N)=O)c(Nc3ccc(C(C)C)cc3)n2)[C@H]1C] | 3-((2R,3R)-1-acryloyl-2- methylpiperidin-3-ylamino)-5-(4- isopropylphenylamino)-1,2,4-triazine- 6-carboxamide |
| 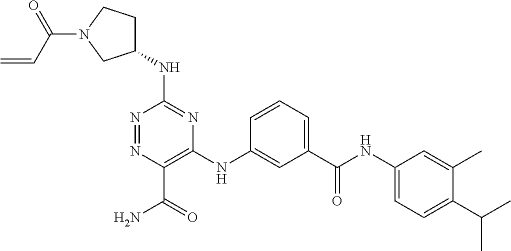 [SMILES: C=CC(=O)N1CC[C@H](Nc2nnc(C(N)=O)c(Nc3cccc(C(=O)Nc4ccc(C(C)C)c(C)c4)c3)n2)C1] | (S)-3-(1-acryloylpyrrolidin-3-ylamino)- 5-(3-(4-isopropyl-3- methylphenylcarbamoyl)phenylamino)- 1,2,4-triazine-6-carboxamide |
| 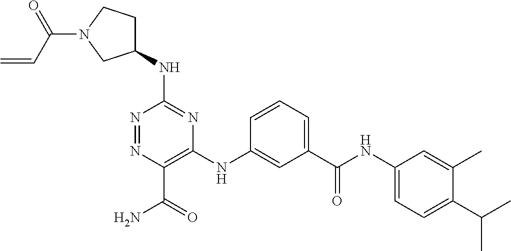 [SMILES: C=CC(=O)N1CC[C@@H](Nc2nnc(C(N)=O)c(Nc3cccc(C(=O)Nc4ccc(C(C)C)c(C)c4)c3)n2)C1] | (R)-3-(1-acryloylpyrrolidin-3- ylamino)-5-(3-(4-isopropyl-3- methylphenylcarbamoyl)phenylamino)- 1,2,4-triazine-6-carboxamide |
| 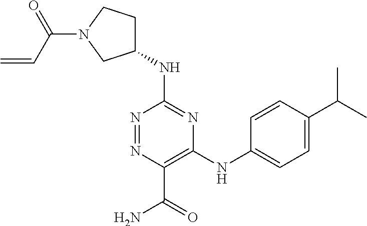 [SMILES: C=CC(=O)N1CC[C@H](Nc2nnc(C(N)=O)c(Nc3ccc(C(C)C)cc3)n2)C1] | (S)-3-(1-acryloylpyrrolidin-3-ylamino)- 5-(4-isopropylphenylamino)-1,2,4- triazine-6-carboxamide |
| 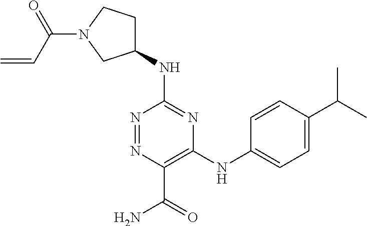 [SMILES: C=CC(=O)N1CC[C@@H](Nc2nnc(C(N)=O)c(Nc3ccc(C(C)C)cc3)n2)C1] | (R)-3-(1-acryloylpyrrolidin-3- ylamino)-5-(4-isopropylphenylamino)- 1,2,4-triazine-6-carboxamide |

or a pharmaceutically acceptable solvate, pharmaceutically acceptable salt, or pharmaceutically acceptable prodrug thereof.

In a particular embodiment, the compound is selected from the group consisting of compounds listed in Table N6:

TABLE N6
(S)-3-(1-acryloylpyrrolidin-3-ylamino)-5-(3-(4-isopropyl-3-
methylphenylcarbamoyl)phenylamino)-1,2,4-triazine-6-carboxamide;
(R)-3-(1-acryloylpyrrolidin-3-ylamino)-5-(3-(4-isopropyl-3-
methylphenylcarbamoyl)phenylamino)-1,2,4-triazine-6-carboxamide;
(S)-3-(1-acryloylpyrrolidin-3-ylamino)-5-(4-isopropylphenylamino)-1,2,4-triazine-6-
carboxamide;
(R)-3-(1-acryloylpyrrolidin-3-ylamino)-5-(4-isopropylphenylamino)-1,2,4-triazine-6-
carboxamide;
3-((2S,3R)-1-acryloyl-2-memylpiperidin-3-ylamino)-5-(4-isopropylphenylamino)-1,2,4-triazine-
6-carboxamide;
(S)-3-(1-acryloylpiperidin-3-ylamino)-5-(4-(oxazol-2-yl)phenylamino)-1,2,4-triazine-6-
carboxamide;
(S)-3-(1-acryloylazepan-3-ylamino)-5-(4-isopropylphenylamino)-1,2,4-triazine-6-carboxamide;
(S)-5-(1-acryloylpiperidin-3-ylamino)-3-(4-(pyrimidin-2-yl)phenylamino)pyrazine-2-
carboxamide;
(S)-5-(1-acryloylpiperidin-3-ylamino)-3-(4-isopropylphenylamino)pyrazine-2-carboxamide;
(R)-3-(1-acryloylazepan-3-ylamino)-5-(4-isopropylphenylamino)-1,2,4-triazine-6-carboxamide;
(S)-3-(1-acryloylpiperidin-3-ylamino)-5-(4-tert-butylphenylamino)-1,2,4-triazine-6-
carboxamide;
(R)-3-(1-acryloylpiperidin-3-ylamino)-5-(4-tert-butylphenylamino)-1,2,4-triazine-6-
carboxamide;
(R)-3-((1-acryloylpyrrolidin-3-yl)(methyl)amino)-5-(4-isopropylphenylamino)-1,2,4-triazine-6-
carboxamide;
(R)-5-((1-acryloylpiperidin-3-yl)(methyl)amino)-3-(4-isopropylphenylamino)pyrazine-2-
carboxamide;
(S)-3-((1-acryloylpiperidin-3-yl)(methyl)amino)-5-(4-isopropylphenylamino)-1,2,4-triazine-6-
carboxamide;
(R)-5-((1-acryloylpiperidin-3-yl)(methyl)amino)-3-(4-(1-cyclopentyl-4-methylpiperidin-4-
yl)phenylamino)pyrazine-2-carboxamide;
(R)-5-(4-(1-acryloyl-4-methylpiperidin-4-yl)phenylamino)-3-(1-acryloylpiperidin-3-ylamino)-
1,2,4-triazine-6-carboxamide;
(R)-5-(4-tert-butylphenylamino)-3-(1-propionylpiperidin-3-ylamino)-1,2,4-triazine-6-
carboxamide;
(R)-3-(1-acryloylpiperidin-3-ylamino)-5-(4-cyclopropylphenylamino)-1,2,4-triazine-6-
carboxamide;
(R)-3-(1-acryloylpiperidin-3-ylamino)-5-(4-(1-cyanocyclopropyl)phenylamino)-1,2,4-triazine-6-
carboxamide;
(R)-3-(N-(1-acryloylpiperidin-3-yl)acrylamido)-5-(4-cyclopropylphenylamino)-1,2,4-triazine-6-
carboxamide;
(R)-3-(N-(1-acryloylpiperidin-3-yl)acrylamido)-5-(4-(1-cyanocyclopropyl)phenylamino)-1,2,4-
triazine-6-carboxamide;
(R)-3-(1-acryloylpiperidin-3-ylamino)-5-(4-(oxazol-2-yl)phenylamino)-1,2,4-triazine-6-
carboxamide;
(R)-3-(1-acryloylpiperidin-3-ylamino)-5-(4-(pyrimidin-2-yl)phenylamino)-1,2,4-triazine-6-
carboxamide;
(R)-3-(1-acryloylpiperidin-3-ylamino)-5-(4-isopropyl-3-methylphenylamino)-1,2,4-triazine-6-
carboxamide;
(R)-3-(1-acryloylpiperidin-3-ylamino)-5-(p-tolylamino)-1,2,4-triazine-6-carboxamide;
(R)-3-(1-acryloylpiperidin-3-ylamino)-5-(m-tolylamino)-1,2,4-triazine-6-carboxamide;
(R)-3-(1-acryloylpiperidin-3-ylamino)-5-(3-methylisothiazol-5-ylamino)-1,2,4-triazine-6-
carboxamide;
(R)-3-(1-acryloylpiperidin-3-ylamino)-5-(4-(1-propionylpiperidin-4-yl)phenylamino)-1,2,4-
triazine-6-carboxamide;
(R)-3-(1-acryloylpiperidin-3-ylamino)-5-(4-(1-cyanocyclopentyl)phenylamino)-1,2,4-triazine-6-
carboxamide;
(R)-3-(1-acryloylpiperidin-3-ylamino)-5-(4-(methylsulfonyl)phenylamino)-1,2,4-triazine-6-
carboxamide;
(R)-3-(1-acryloylpiperidin-3-ylamino)-5-(4-(1-cyclopentylpiperidin-4-yl)phenylamino)-1,2,4-
triazine-6-carboxamide;
(R)-3-(1-acryloylpiperidin-3-ylamino)-5-(4-(2-cyanopropan-2-yl)phenylamino)-1,2,4-triazine-6-
carboxamide;
(R)-3-(1-acryloylpiperidin-3-ylamino)-5-(4-iodophenylamino)-1,2,4-triazine-6-carboxamide;
(R)-3-(1-acryloylpiperidin-3-ylamino)-5-(benzo[d]thiazol-6-ylamino)-1,2,4-triazine-6-
carboxamide;
(R)-5-(4-((1H-1,2,4-triazol-1-yl)methyl)phenylamino)-3-(1-acryloylpiperidin-3-ylamino)-1,2,4-
triazine-6-carboxamide;
(R)-5-((1-acryloylpyrrolidin-3-yl)(methyl)amino)-3-(4-(1-cyclopropyl-4-methylpiperidin-4-
yl)phenylamino)pyrazine-2-carboxamide;
(R)-3-(4-(1-cyclopropyl-4-methylpiperidin-4-yl)phenylamino)-5-(methyl(1-(2,2,2-
trifluoroacetyl)pyrrolidin-3-yl)amino)pyrazine-2-carboxamide;
(R)-5-((1-acryloylpyrrolidin-3-yl)(methyl)amino)-3-(4-(1-cyclopentyl-4-methylpiperidin-4-
yl)phenylamino)pyrazine-2-carboxamide;
(R)-3-(4-(1-cyclopentyl-4-methylpiperidin-4-yl)phenylamino)-5-(rnethyl(1-(2,2,2-
trifluoroacetyl)pyrrolidin-3-yl)amino)pyrazine-2-carboxamide;
(R)-3-(1-acryloylpiperidin-3-ylamino)-5-(5-fluoropyridin-3-ylamino)-1,2,4-triazine-6-
carboxamide;
(R)-3-(1-acryloylpiperidin-3-ylamino)-5-(quinolin-3-ylamino)-1,2,4-triazine-6-carboxamide;
(R)-3-(1-acryloylpiperidin-3-ylamino)-5-(3-(pyrimidin-2-yl)phenylamino)-1,2,4-triazine-6-
carboxamide;
benzyl 4-(3-((R)-1-acryloylpiperidin-3-ylamino)-6-carbamoyl-1,2,4-triazin-5-
ylamino)benzyl((S)-3,3-dimethylbutan-2-yl)carbamate;
3-((R)-1-acryloylpiperidin-3-ylamino)-5-(4-(((S)-3,3-dimethylbutan-2-
ylamino)methyl)phenylamino)-1,2,4-triazine-6-carboxamide;
(R)-5-(4-(1-acryloyl-1H-pyrazol-4-yl)phenylamino)-3-(1-acryloylpiperidin-3-ylamino)-1,2,4-
triazine-6-carboxamide;
(R)-5-(3-(2H-1,2,3-triazol-2-yl)phenylamino)-3-(1-acryloylpiperidin-3-ylamino)-1,2,4-triazine-
6-carboxamide;
(R)-3-(1-acryloylpiperidin-3-ylamino)-5-(4-(3-oxomorpholino)phenylamino)-1,2,4-triazine-6-
carboxamide;
(R)-3-(1-acryloylpiperidin-3-ylamino)-5-(4-(thiazol-2-yl)phenylamino)-1,2,4-triazine-6-
carboxamide;
(R)-5-(4-(2H-tetrazol-5-yl)phenylamino)-3-(1-acryloylpiperidin-3-ylamino)-1,2,4-triazine-6-
carboxamide
(R)-3-(1-acryloylpiperidin-3-ylamino)-5-(3-(oxazol-2-yl)phenylamino)-1,2,4-triazine-6-
carboxamide;
(R)-3-(1-acryloylpiperidin-3-ylamino)-5-(1-ethyl-1H-indazol-5-ylamino)-1,2,4-triazine-6-
carboxamide;
(R)-5-(4-(2H-1,2,3-triazol-2-yl)phenylamino)-3-(1-acryloylpiperidin-3-ylamino)-1,2,4-triazine-
6-carboxamide
(R)-3-(1-acryloylpiperidin-3-ylamino)-5-(quinolin-6-ylamino)-1,2,4-triazine-6-carboxamide;
(R)-5-(4-(1H-1,2,4-triazol-1-yl)phenylamino)-3-(1-acryloylpiperidin-3-ylamino)-1,2,4-triazine
6-carboxamide;
(R)-5-(1-acryloylpiperidin-3-ylamino)-3-(4-(1-cyclopentylpiperidin-4-yl)phenylamino)pyrazine-
2-carboxamide;
(R)-5-((1-acryloylpyrrolidin-3-yl)(methyl)amino)-3-(4-(1-cyclopentylpiperidin-4-
yl)phenylamino)pyrazine-2-carboxamide;
(R)-3-(1-acryloylpiperidin-3-ylamino)-5-(3-(thiazol-2-yl)phenylamino)-1,2,4-triazine-6-
carboxamide;
(R)-3-(1-acryloylpiperidin-3-ylamino)-5-(1-methyl-2-oxo-1,2,3,4-tetrahydroquinolin-6-
ylamino)-1,2,4-triazine-6-carboxamide;
5-((2R,3R)-1-acryloyl-2-methylpiperidin-3-ylamino)-3-(4-(1-cyclopentylpiperidin-4-
yl)phenylamino)pyrazine-2-carboxamide; and
5-((2R,3R)-1-acryloyl-2-methylpiperidin-3-ylamino)-3-(4-(1-cyclopentyl-4-methylpiperidin-4-
yl)phenylamino)pyrazine-2-carboxamide;

or a pharmaceutically acceptable solvate, pharmaceutically acceptable salt, or pharmaceutically acceptable prodrug thereof.

In another particular embodiment, the compound is selected from the group consisting of compounds listed in Table N7:

TABLE N7

| Ex. # | Chemical Name |
|---|---|
| D-1 | 5-((2R,3R)-3-(4-cyclopropyl-2-fluorobenzamido)-2-methylpiperidin-1-yl)-3-((1-methyl- |
| | 1H-pyrazol-4-yl)amino)pyrazine-2-carboxamide |
| D-2 | (R)-5-(3-(4-cyclopropyl-2-fluorobenzamido)piperidin-1-yl)-3-((3-methylisoxazol-5- |
| | yl)amino)pyrazine-2-carboxamide |
| D-3 | (R)-5-(3-(4-cyclopropyl-2-fluorobenzamido)piperidin-1-yl)-3-((3-phenylisothiazol-5- |
| | yl)amino)pyrazine-2-carboxamide |
| D-4 | (R)-5-(3-(4-cyclopropyl-2-fluorobenzamido)piperidin-1-yl)-3-((2-methylthiazol-5- |
| | yl)amino)pyrazine-2-carboxamide |
| D-5 | 3-((4-(4-cyclopentylpiperazin-1-yl)phenyl)amino)-5-((2R,3R)-3-(4-cyclopropyl-2- |
| | fluorobenzamido)-2-methylpiperidin-1-yl)pyrazine-2-carboxamide |
| D-6 | 5-((2R,3R)-3-(6-cyclopropyl-1-oxoisoquinolin-2(1H)-yl)-2-methylpiperidin-1-yl)-3-((4- |
| | (4-methylpiperazin-1-yl)phenyl)amino)pyrazine-2-carboxamide |
| D-7 | 5-((2R,3R)-3-(4-cyclopropyl-2-fluorobenzamido)-2-methylpiperidin-1-yl)-3-((4-(4- |
| | isopropylpiperazin-1-yl)phenyl)amino)pyrazine-2-carboxamide |
| D-8 | 5-((2R,3R)-3-(4-cyclopropyl-2-fluorobenzamido)-2-methylpiperidin-1-yl)-3-((5-(4- |
| | methylpiperazin-1-yl)pyridin-2-yl)amino)pyrazine-2-carboxamide |
| D-9 | 3-((4-(1-cyclopentyl-4-methylpiperidin-4-yl)phenyl)amino)-5-((2R,3R)-2-methyl-3-(3- |
| | (pyridin-3-yl)ureido)piperidin-1-yl)pyrazine-2-carboxamide |
| D-10 | 5-((2R,3R)-3-(4-cyclopropyl-2-fluorobenzamido)-2-methylpiperidin-1-yl)-3-((4-(4- |
| | methyl-2-oxopiperazin-1-yl)phenyl)amino)pyrazine-2-carboxamide |
| D-11 | 5-((2R,3R)-2-methyl-3-(4-(trifluoromethyl)benzamido)piperidin-1-yl)-3-((4-(4- |
| | methylpiperazin-1-yl)phenyl)amino)pyrazine-2-carboxamide |
| D-12 | 5-((2R,3R)-3-(4-cyclopropyl-2-fluorobenzamido)-2-methylpiperidin-1-yl)-3-((4-((S)- |
| | 2,4-dimethylpiperazin-1-yl)phenyl)amino)pyrazine-2-carboxamide |
| D-13 | 5-((2R,3R)-3-(4-cyclopropyl-2-fluorobenzamido)-2-methylpiperidin-1-yl)-3-((6-(4- |
| | methylpiperazin-1-yl)pyridin-3-yl)amino)pyrazine-2-carboxamide |
| D-14 | 5-((2R,3R)-3-(4-(tert-butyl)benzamido)-2-methylpiperidin-1-yl)-3-((4-(4- |
| | methylpiperazin-1-yl)phenyl)amino)pyrazine-2-carboxamide |
| D-15 | (R)-N-(1-(5-carbamoyl-6-((3-methylisothiazol-5-yl)amino)pyrazin-2-yl)piperidin-3-yl)- |
| | 1-oxoisoindoline-2-carboxamide |
| D-16 | 5-((2R,3R)-3-(2-chloro-4-cyclopropylbenzamido)-2-methylpiperidin-1-yl)-3-((4-(4- |
| | methylpiperazin-1-yl)phenyl)amino)pyrazine-2-carboxamide |
| D-17 | 5-((2R,3R)-3-(3-chloro-4-cyclopropylbenzamido)-2-methylpiperidin-1-yl)-3-((4-(4- |
| | methylpiperazin-1-yl)phenyl)amino)pyrazine-2-carboxamide |
| D-18 | 5-((2R,3R)-3-(6-cyclopropylnicotinamido)-2-methylpiperidin-1-yl)-3-((1-methyl-1H- |
| | pyrazol-4-yl)amino)pyrazine-2-carboxamide |
| D-19 | N-((2R,3R)-1-(5-carbamoyl-6-((1-methyl-1H-pyrazol-4-yl)amino)pyrazin-2-yl)-2- |
| | methylpiperidin-3-yl)-1,5,5-trimethyl-1,4,5,6-tetrahydrocyclopenta[b]pyrrole-2- |
| | carboxamide |
| D-20 | 5-((2R,3R)-3-(2-chloro-4-cyclopropylbenzamido)-2-methylpiperidin-1-yl)-3-((1-methyl- |
| | 1H-pyrazol-4-yl)amino)pyrazine-2-carboxamide |
| D-21 | 5-((2R,3R)-3-(5-cyclopropylpicolinamido)-2-methylpiperidin-1-yl)-3-((1-methyl-1H- |
| | pyrazol-4-yl)amino)pyrazine-2-carboxamide |
| D-22 | 5-((2R,3R)-3-(4-(tert-butyl)benzamido)-2-methylpiperidin-1-yl)-3-((1-methyl-1H- |
| | pyrazol-4-yl)amino)pyrazine-2-carboxamide |
| D-23 | 3-((1-methyl-1H-pyrazol-4-yl)amino)-5-((2R,3R)-2-methyl-3-(4-(oxetan-3- |
| | yl)benzamido)piperidin-1-yl)pyrazine-2-carboxamide |
| D-24 | 5-((2R,3R)-3-(4-(dimethylamino)benzamido)-2-methylpiperidin-1-yl)-3-((1-methyl-1H- |
| | pyrazol-4-yl)amino)pyrazine-2-carboxamide |
| D-25 | 5-((2R,3R)-3-(4-cyclopropyl-2-(trifluoromethyl)benzamido)-2-methylpiperidin-1-yl)-3- |
| | ((1-methyl-1H-pyrazol-4-yl)amino)pyrazine-2-carboxamide |
| D-26 | 5-(3-(4-cyclopropylbenzamido)-4-hydroxypiperidin-1-yl)-3-((1-methyl-1H-pyrazol-4- |
| | yl)amino)pyrazine-2-carboxamide |
| D-27 | 5-((2R,3R)-3-(5-bromo-1-oxoisoindolin-2-yl)-2-methylpiperidin-1-yl)-3-((1-methyl-1H- |
| | pyrazol-4-yl)amino)pyrazine-2-carboxamide |
| D-28 | 5-((3S,4S)-4-(4-cyclopropylbenzamido)-3-hydroxypiperidin-1-yl)-3-((1-methyl-1H- |
| | pyrazol-4-yl)amino)pyrazine-2-carboxamide |
| D-29 | 5-((3R,4S)-4-(4-cyclopropylbenzamido)-3-hydroxypiperidin-1-yl)-3-((1-methyl-1H- |
| | pyrazol-4-yl)amino)pyrazine-2-carboxamide |
| D-30 | 5-((3R,5S)-3-(4-cyclopropylbenzamido)-5-methylpiperidin-1-yl)-3-((1-methyl-1H- |
| | pyrazol-4-yl)amino)pyrazine-2-carboxamide |
| D-31 | 5-((3S,5S)-3-(4-cyclopropylbenzamido)-5-methylpiperidin-1-yl)-3-((1-methyl-1H- |
| | pyrazol-4-yl)amino)pyrazine-2-carboxamide |
| D-32 | 5-((3S,5R)-3-(4-cyclopropylbenzamido)-5-methylpiperidin-1-yl)-3-((1-methyl-1H- |
| | pyrazol-4-yl)amino)pyrazine-2-carboxamide |
| D-33 | 5-((2R,3R)-3-(3,3-dimethylureido)-2-methylpiperidin-1-yl)-3-((4-((1-methylpiperidin-4- |
| | yl)oxy)phenyl)amino)pyrazine-2-carboxamide |
| D-34 | 5-((3R,5R)-3-(4-cyclopropylbenzamido)-5-methylpiperidin-1-yl)-3-((1-methyl-1H- |
| | pyrazol-4-yl)amino)pyrazine-2-carboxamide |
| D-35 | 5-((2R,3R)-3-(1-isopropyl-2-oxo-1,2-dihydropyridine-4-carboxamido)-2- |
| | methylpiperidin-1-yl)-3-((1-methyl-1H-pyrazol-4-yl)amino)pyrazine-2-carboxamide |
| D-36 | 5-((2R,3R)-3-(5-cyclopropyl-1-oxoisoindolin-2-yl)-2-methylpiperidin-1-yl)-3-((1- |
| | methyl-1H-pyrazol-4-yl)amino)pyrazine-2-carboxamide |
| D-37 | 5-((2R,3R)-3-(4-cyclopropylbenzamido)-2-methylpiperidin-1-yl)-3-((4-(piperidin-1- |
| | yl)phenyl)amino)pyrazine-2-carboxamide |
| D-38 | 3-((4-(1-cyclopropyl-4-methylpiperidin-4-yl)phenyl)amino)-5-((2R,3R)-2-methyl-3-(3- |
| | methyl-2-oxoimidazolidin-1-yl)piperidin-1-yl)pyrazine-2-carboxamide |
| D-39 | (R)-3-(3-(4-(2-hydroxypropan-2-yl)benzamido)piperidin-1-yl)-5-((1-methyl-1H-pyrazol- |
| | 4-yl)amino)-1,2,4-triazine-6-carboxamide |
| D-40 | 5-((2R,3R)-3-(6-(2-hydroxypropan-2-yl)nicotinamido)-2-methylpiperidin-1-yl)-3-((1- |
| | methyl-1H-pyrazol-4-yl)amino)pyrazine-2-carboxamide |
| D-41 | 3-((2R,3R)-3-(6-(2-hydroxypropan-2-yl)nicotinamido)-2-methylpiperidin-1-yl)-5-((1- |
| | methyl-1H-pyrazol-4-yl)amino)-1,2,4-triazine-6-carboxamide |
| D-42 | 5-((2R,3R)-3-(4-cyclopropylbenzamido)-2-methylpiperidin-1-yl)-3-((4- |
| | (trifluoromethoxy)phenyl)amino)pyrazine-2-carboxamide |
| D-43 | 5-((3R,5S)-3-(4-cyclopropylbenzamido)-5-(hydroxymethyl)piperidin-1-yl)-3-((1-methyl- |
| | 1H-pyrazol-4-yl)amino)pyrazine-2-carboxamide |
| D-44 | 5-((3aR,7aR)-1-(4-cyclopropylbenzoyl)octahydro-4H-pyrrolo[3,2-b]pyridin-4-yl)-3-((1- |
| | methyl-1H-pyrazol-4-yl)amino)pyrazine-2-carboxamide |
| D-45 | 5-((3aS,7aS)-1-(4-cyclopropylbenzoyl)octahydro-4H-pyrrolo[3,2-b]pyridin-4-yl)-3-((1- |
| | methyl-1H-pyrazol-4-yl)amino)pyrazine-2-carboxamide |
| D-46 | 5-((4aR,8aR)-5-(4-cyclopropylbenzoyl)octahydro-1,5-naphthyridin-1(2H)-yl)-3-((1- |
| | methyl-1H-pyrazol-4-yl)amino)pyrazine-2-carboxamide |
| D-47 | 5-((4aS,8aS)-5-(4-cyclopropylbenzoyl)octahydro-1,5-naphthyridin-1(2H)-yl)-3-((1- |
| | methyl-1H-pyrazol-4-yl)amino)pyrazine-2-carboxamide |
| D-48 | 5-((2R,3R)-3-(6-cyclopropylnicotinamido)-2-methylpiperidin-1-yl)-3-((4-((4,4- |
| | difluorocyclohexyl)oxy)phenyl)amino)pyrazine-2-carboxamide |
| D-49 | 5-((3R,5R)-3-(4-cyclopropylbenzamido)-5-(hydroxymethyl)piperidin-1-yl)-3-((1- |
| | methyl-1H-pyrazol-4-yl)amino)pyrazine-2-carboxamide |
| D-50 | 4-(5-carbamoyl-6-((1-methyl-1H-pyrazol-4-yl)amino)pyrazin-2-yl)-N,N- |
| | dimethyloctahydro-1H-pyrrolo[3,2-b]pyridine-1-carboxamide |
| D-51 | 5-((2R,3R)-3-(4-(2-aminopropan-2-yl)benzamido)-2-methylpiperidin-1-yl)-3-((1- |
| | methyl-1H-pyrazol-4-yl)amino)pyrazine-2-carboxamide |
| D-52 | 5-((2R,3R)-3-(3,3-dimethylureido)-2-methylpiperidin-1-yl)-3-((2-fluoro-4-(4- |
| | methylpiperazin-1-yl)phenyl)amino)pyrazine-2-carboxamide |
| D-53 | 3-((1H-pyrazol-4-yl)amino)-5-((2R,3R)-3-(3,3-dimethylureido)-2-methylpiperidin-1- |
| | yl)pyrazine-2-carboxamide |
| D-54 | 5-(1-benzoyloctahydro-4H-pyrrolo[3,2-b]pyridin-4-yl)-3-((1-methyl-1H-pyrazol-4- |
| | yl)amino)pyrazine-2-carboxamide |
| D-55 | 5-((2R,3R)-3-(3,3-dimethyl-2,3-dihydrobenzofuran-5-carboxamido)-2-methylpiperidin- |
| | 1-yl)-3-((1-methyl-1H-pyrazol-4-yl)amino)pyrazine-2-carboxamide |
| D-56 | 3-((1-methyl-1H-pyrazol-4-yl)amino)-5-((2R,3R)-2-methyl-3-(3-methylchromane-7- |
| | carboxamido)piperidin-1-yl)pyrazine-2-carboxamide |
| D-57 | 5-((2R,3R)-3-(1-(tert-butyl)-1H-pyrazole-4-carboxamido)-2-methylpiperidin-1-yl)-3-((1- |
| | methyl-1H-pyrazol-4-yl)amino)pyrazine-2-carboxamide |
| D-58 | 3-((4-(1-cyclopentyl-4-methylpiperidin-4-yl)phenyl)amino)-5-((2S,3R)-3-(3,3- |
| | dimethylureido)-2-(hydroxymethyl)piperidin-1-yl)pyrazine-2-carboxamide |
| D-59 | 3-((4-(1-cyclopentyl-4-methylpiperidin-4-yl)phenyl)amino)-5-((2R,3S)-3-(3,3- |
| | dimethylureido)-2-(hydroxymethyl)piperidin-1-yl)pyrazine-2-carboxamide |
| D-60 | 3-((4-(1-cyclopentyl-4-methylpiperidin-4-yl)phenyl)amino)-5-((2R,3R)-3-(3,3- |
| | dimethylureido)-2-(hydroxymethyl)piperidin-1-yl)pyrazine-2-carboxamide |
| D-61 | 3-((4-(1-cyclopentyl-4-methylpiperidin-4-yl)phenyl)amino)-5-((2S,3S)-3-(3,3- |
| | dimethylureido)-2-(hydroxymethyl)piperidin-1-yl)pyrazine-2-carboxamide |
| D-62 | 5-((2R,3R)-3-(4-cyclopropyl-2-fluorobenzamido)-2-methylpiperidin-1-yl)-3-((5-methyl- |
| | 4,5,6,7-tetrahydrothiazolo[5,4-c]pyridin-2-yl)amino)pyrazine-2-carboxamide |
| D-63 | 5-((2R,3R)-3-(4-cyclopropyl-2-fluorobenzamido)-2-methylpiperidin-1-yl)-3-((4- |
| | methylthiazol-2-yl)amino)pyrazine-2-carboxamide |
| D-64 | 5-((2S,3S)-3-(3,3-dimethylureido)-2-(hydroxymethyl)piperidin-1-yl)-3-(quinolin-6- |
| | ylamino)pyrazine-2-carboxamide |
| D-65 | 5-((2R,3S)-3-(3,3-dimethylureido)-2-(hydroxymethyl)piperidin-1-yl)-3-(quinolin-6- |
| | ylamino)pyrazine-2-carboxamide |
| D-66 | 3-((4-(1-cyclopentyl-4-methylpiperidin-4-yl)phenyl)amino)-5-((2R,3R)-3-(4- |
| | cyclopropyl-2-fluorobenzamido)-2-methylpiperidin-1-yl)pyrazine-2-carboxamide |
| D-67 | 5-((2S,3R)-3-(3,3-dimethylureido)-2-(hydroxymethyl)piperidin-1-yl)-3-((3- |
| | methylisothiazol-5-yl)amino)pyrazine-2-carboxamide |
| D-68 | 5-((2R,3S)-3-(3,3-dimethylureido)-2-(hydroxymethyl)piperidin-1-yl)-3-((3- |
| | methylisothiazol-5-yl)amino)pyrazine-2-carboxamide |
| D-69 | 5-((4-(3,3-dimethylureido)cyclohexyl)amino)-3-((4-(4-methylpiperazine-1- |
| | carbonyl)phenyl)amino)pyrazine-2-carboxamide |
| D-70 | 5-(((1r,4r)-4-(4-cyclopropyl-2-fluorobenzamido)cyclohexyl)amino)-3-((4-(4- |
| | methylpiperazine-1-carbonyl)phenyl)amino)pyrazine-2-carboxamide |
| D-71 | 5-((4-(4-cyclopropyl-2-fluorobenzamido)cyclohexyl)amino)-3-((4-(4-methylpiperazine- |
| | 1-carbonyl)phenyl)amino)pyrazine-2-carboxamide |
| D-72 | 5-(((1s,4s)-4-(3,3-dimethylureido)cyclohexyl)amino)-3-((4-(4-methylpiperazine-1- |
| | carbonyl)phenyl)amino)pyrazine-2-carboxamide |
| D-73 | 5-((2R,3R)-3-(4-cyclopropyl-2-fluorobenzamido)-2-methylpiperidin-1-yl)-3-((4- |
| | morpholinophenyl)amino)pyrazine-2-carboxamide |
| D-74 | 5-((2S,5R)-5-(3,3-dimethylureido)-2-methylpiperidin-1-yl)-3-((3-methylisothiazol-5- |
| | yl)amino)pyrazine-2-carboxamide |
| D-75 | 5-((2R,5S)-5-(3,3-dimethylureido)-2-methylpiperidin-1-yl)-3-((3-methylisothiazol-5- |
| | yl)amino)pyrazine-2-carboxamide |
| D-76 | 5-((2R,5R)-5-(3,3-dimethylureido)-2-methylpiperidin-1-yl)-3-((3-methylisothiazol-5- |
| | yl)amino)pyrazine-2-carboxamide |
| D-77 | 5-((2S,5S)-5-(3,3-dimethylureido)-2-methylpiperidin-1-yl)-3-((3-methylisothiazol-5- |
| | yl)amino)pyrazine-2-carboxamide |
| D-78 | 5-((2R,5R)-5-(4-cyclopropyl-2-fluorobenzamido)-2-methylpiperidin-1-yl)-3-((3- |
| | methylisothiazol-5-yl)amino)pyrazine-2-carboxamide |
| D-79 | 5-((2S,5S)-5-(4-cyclopropyl-2-fluorobenzamido)-2-methylpiperidin-1-yl)-3-((3- |
| | methylisothiazol-5-yl)amino)pyrazine-2-carboxamide |
| D-80 | 5-((2R,5S)-5-(4-cyclopropyl-2-fluorobenzamido)-2-methylpiperidin-1-yl)-3-((3- |
| | methylisothiazol-5-yl)amino)pyrazine-2-carboxamide |
| D-81 | 5-((2R,3R)-3-(4-cyclopropylbenzamido)-2-methylpiperidin-1-yl)-3-((4-(4- |
| | methylpiperazin-1-yl)phenyl)amino)pyrazine-2-carboxamide |
| D-82 | 5-((2R,3R)-3-(4-cyclopropyl-2-fluorobenzamido)-2-methylpiperidin-1-yl)-3-((3-fluoro- |
| | 4-(4-methylpiperazin-1-yl)phenyl)amino)pyrazine-2-carboxamide |
| D-83 | 5-((2R,3R)-3-(4-cyclopropylbenzamido)-2-methylpiperidin-1-yl)-3-(imidazo[1,2- |
| | a]pyridin-6-ylamino)pyrazine-2-carboxamide |
| D-84 | 5-((2R,3R)-3-(5-(dimethylamino)picolinamido)-2-methylpiperidin-1-yl)-3-((1-methyl- |
| | 1H-pyrazol-4-yl)amino)pyrazine-2-carboxamide |
| D-85 | 3-((1-methyl-1H-pyrazol-4-yl)amino)-5-((2R,3R)-2-methyl-3-(4-(pyrimidin-2- |
| | yl)benzamido)piperidin-1-yl)pyrazine-2-carboxamide |
| D-86 | N-((2R,3R)-1-(5-carbamoyl-6-((1-methyl-1H-pyrazol-4-yl)amino)pyrazin-2-yl)-2- |
| | methylpiperidin-3-yl)-2-cyclopropylpyrimidine-5-carboxamide |
| D-87 | 3-((1-methyl-1H-pyrazol-4-yl)amino)-5-((2R,3R)-2-methyl-3-(5- |
| | (trifluoromethyl)picolinamido)piperidin-1-yl)pyrazine-2-carboxamide |
| D-88 | 5-((2R,3R)-3-(4-(2-cyanopropan-2-yl)benzamido)-2-methylpiperidin-1-yl)-3-((1-methyl- |
| | 1H-pyrazol-4-yl)amino)pyrazine-2-carboxamide |
| D-89 | 5-((2R,3R)-3-(4-cyclopropylbenzamido)-2-methylpiperidin-1-yl)-3-((4-((1- |
| | methylpiperidin-4-yl)oxy)phenyl)amino)pyrazine-2-carboxamide |
| D-90 | 5-((2R,3R)-3-(bicyclo[1.1.1]pentane-1-carboxamido)-2-methylpiperidin-1-yl)-3-((1- |
| | methyl-1H-pyrazol-4-yl)amino)pyrazine-2-carboxamide |
| D-91 | 3-((1-methyl-1H-pyrazol-4-yl)amino)-5-((2R,3R)-2-methyl-3-(6- |
| | (trifluoromethyl)nicotinamido)piperidin-1-yl)pyrazine-2-carboxamide |
| D-92 | 5-((2R,3R)-3-(4-cyclopropylbenzamido)-2-methylpiperidin-1-yl)-3-((4-(4- |
| | methylpiperazin-1-yl)-3-(trifluoromethyl)phenyl)amino)pyrazine-2-carboxamide |
| D-93 | 3-((1-methyl-1H-pyrazol-4-yl)amino)-5-((2R,3R)-2-methyl-3-(3-methyl-2- |
| | oxoimidazolidin-1-yl)piperidin-1-yl)pyrazine-2-carboxamide |
| D-94 | 5-((2R,3R)-3-(4-(1-hydroxy-2-methylpropan-2-yl)benzamido)-2-methylpiperidin-1-yl)- |
| | 3-((1-methyl-1H-pyrazol-4-yl)amino)pyrazine-2-carboxamide |
| D-95 | 3-((1-methyl-1H-pyrazol-4-yl)amino)-5-((2R,3R)-2-methyl-3-(4- |
| | (trifluoromethoxy)benzamido)piperidin-1-yl)pyrazine-2-carboxamide |
| D-96 | 5-((2R,3R)-3-(4-cyclopropylbenzamido)-2-methylpiperidin-1-yl)-3-((4-(4,4- |
| | difluoropiperidin-1-yl)phenyl)amino)pyrazine-2-carboxamide |
| D-97 | 5-((2R,3R)-3-(4-cyclopropylbenzamido)-2-methylpiperidin-1-yl)-3-((4-(4-(2,2,2- |
| | trifluoroethyl)piperazin-1-yl)phenyl)amino)pyrazine-2-carboxamide |
| D-98 | 5-((2R,3R)-3-(4-cyclopropylbenzamido)-2-methylpiperidin-1-yl)-3-((4-((1-(2,2,2- |
| | trifluoroethyl)piperidin-4-yl)oxy)phenyl)amino)pyrazine-2-carboxamide |
| D-99 | 3-((4-(2-oxa-7-azaspiro[3.5]nonan-7-yl)phenyl)amino)-5-((2R,3R)-3-(4- |
| | cyclopropylbenzamido)-2-methylpiperidin-1-yl)pyrazine-2-carboxamide |
| D-100 | 5-((2R,3R)-3-(4-cyclopropylbenzamido)-2-methylpiperidin-1-yl)-3-((4-(4-(2- |
| | hydroxypropan-2-yl)piperidin-1-yl)phenyl)amino)pyrazine-2-carboxamide |
| D-101 | 3-((4-((1-cyclopentylpiperidin-4-yl)oxy)phenyl)amino)-5-((2R,3R)-3-(4- |
| | cyclopropylbenzamido)-2-methylpiperidin-1-yl)pyrazine-2-carboxamide |
| D-102 | 3-((4-((1-cyclopentylpiperidin-4-yl)oxy)phenyl)amino)-5-((2R,3R)-3-(3,3- |
| | dimethylureido)-2-methylpiperidin-1-yl)pyrazine-2-carboxamide |
| D-103 | 5-((2R,3R)-3-(4-cyclopropylbenzamido)-2-methylpiperidin-1-yl)-3-((2-fluoro-4-(4- |
| | methylpiperazin-1-yl)phenyl)amino)pyrazine-2-carboxamide |
| D-104 | 5-((2R,3R)-3-(4-cyclopropylbenzamido)-2-methylpiperidin-1-yl)-3-((4-(((1R,3s,5S)-8- |
| | methyl-8-azabicyclo[3.2.1]octan-3-yl)oxy)phenyl)amino)pyrazine-2-carboxamide |
| D-105 | 5-((2R,3R)-3-(4-cyclopropylbenzamido)-2-methylpiperidin-1-yl)-3-((4-((1- |
| | methylpiperidin-3-yl)oxy)phenyl)amino)pyrazine-2-carboxamide |
| D-106 | 5-((2R,3R)-3-(4-cyclopropylbenzamido)-2-methylpiperidin-1-yl)-3-((4-(((1R,5S)-8- |
| | methyl-8-azabicyclo[3.2.1]octan-3-yl)oxy)phenyl)amino)pyrazine-2-carboxamide |
| D-107 | 5-((2R,3R)-3-(4-(2-acetamidopropan-2-yl)benzamido)-2-methylpiperidin-1-yl)-3-((1- |
| | methyl-1H-pyrazol-4-yl)amino)pyrazine-2-carboxamide |
| D-108 | 5-((2R,3R)-3-(4-(2-(dimethylamino)propan-2-yl)benzamido)-2-methylpiperidin-1-yl)-3- |
| | ((1-methyl-1H-pyrazol-4-yl)amino)pyrazine-2-carboxamide |
| D-109 | 3-((1-methyl-1H-pyrazol-4-yl)amino)-5-((2R,3R)-2-methyl-3-(3-methylureido)piperidin- |
| | 1-yl)pyrazine-2-carboxamide |
| D-110 | 5-((2R,3R)-3-(3,3-dimethylureido)-2-methylpiperidin-1-yl)-3-((4-(4-methylpiperazine-1- |
| | carbonyl)phenyl)amino)pyrazine-2-carboxamide |
| D-111 | 5-((2R,3R)-3-(3,3-dimethylureido)-2-methylpiperidin-1-yl)-3-((4-(4-methylpiperazin-1- |
| | yl)phenyl)amino)pyrazine-2-carboxamide |
| D-112 | 3-((4-(1-cyclopentyl-4-methylpiperidin-4-yl)phenyl)amino)-5-((2R,3R)-3-(4- |
| | cyclopropylbenzamido)-2-methylpiperidin-1-yl)pyrazine-2-carboxamide |
| D-113 | 5-((2R,3R)-3-(4-cyclopropyl-2-fluorobenzamido)-2-methylpiperidin-1-yl)-3-((4-((4- |
| | methylpiperazin-1-yl)methyl)phenyl)amino)pyrazine-2-carboxamide |
| D-114 | 5-((2R,3R)-3-(4-cyclopropyl-2-fluorobenzamido)-2-methylpiperidin-1-yl)-3-(quinolin-6- |
| | ylamino)pyrazine-2-carboxamide |
| D-115 | 5-((2R,3R)-3-(4-cyclopropyl-2-fluorobenzamido)-2-methylpiperidin-1-yl)-3-((4-(4- |
| | methylpiperazine-1-carbonyl)phenyl)amino)pyrazine-2-carboxamide |
| D-116 | 5-((2R,3R)-3-(4-cyclopropyl-2-fluorobenzamido)-2-methylpiperidin-1-yl)-3-((3- |
| | methylisothiazol-5-yl)amino)picolinamide |
| D-117 | 5-((2R,3R)-3-(4-cyclopropyl-2-fluorobenzamido)-2-methylpiperidin-1-yl)-3-((1-methyl- |
| | 1H-pyrazol-4-yl)amino)picolinamide |
| D-118 | 5-((2R,3R)-3-(3,3-dimethylureido)-2-methylpiperidin-1-yl)-3-((3- |
| | (morpholinomethyl)isothiazol-5-yl)amino)pyrazine-2-carboxamide |
| D-119 | 3-((3,4-dimethylisothiazol-5-yl)amino)-5-((2R,3R)-3-(3,3-dimethylureido)-2- |
| | methylpiperidin-1-yl)pyrazine-2-carboxamide |
| D-120 | 5-((2R,3R)-3-(3,3-dimethylureido)-2-methylpiperidin-1-yl)-3-((4-((4-methylpiperazin-1- |
| | yl)methyl)phenyl)amino)picolinamide |
| D-121 | 5-((2R,3R)-3-(3,3-dimethylureido)-2-methylpiperidin-1-yl)-3-(quinolin-7- |
| | ylamino)pyrazine-2-carboxamide |
| D-122 | 5-((2R,3R)-3-(4-cyclopropyl-2-fluorobenzamido)-2-methylpiperidin-1-yl)-3-((3- |
| | ((dimethylamino)methyl)isothiazol-5-yl)amino)pyrazine-2-carboxamide |
| D-123 | 5-((2R,3R)-3-(4-cyclopropyl-2-fluorobenzamido)-2-methylpiperidin-1-yl)-3-((1- |
| | isopropyl-1H-pyrazol-4-yl)amino)pyrazine-2-carboxamide |
| D-124 | 5-((2R,3R)-3-(4-cyclopropyl-2-fluorobenzamido)-2-methylpiperidin-1-yl)-3-(quinolin-7- |
| | ylamino)pyrazine-2-carboxamide |
| D-125 | 5-((2R,3R)-3-(4-cyclopropyl-2-fluorobenzamido)-2-methylpiperidin-1-yl)-3-((4-((4- |
| | methylpiperazin-1-yl)methyl)phenyl)amino)picolinamide |
| D-126 | 5-((2R,3R)-3-(4-cyclopropyl-2-fluorobenzamido)-2-methylpiperidin-1-yl)-3-((4-(4- |
| | methylpiperazin-1-yl)phenyl)amino)picolinamide |
| D-127 | 5-((2R,3R)-3-(3,3-dimethylureido)-2-methylpiperidin-1-yl)-3-((4-(4-methylpiperazin-1- |
| | yl)phenyl)amino)picolinamide |
| D-128 | 5-((2R,3R)-3-(3,3-dimethylureido)-2-methylpiperidin-1-yl)-3-((1-methyl-1H-pyrazol-4- |
| | yl)amino)pyrazine-2-carboxamide |
| D-129 | 5-((2R,3R)-3-(4-cyclopropyl-2-fluorobenzamido)-2-methylpiperidin-1-yl)-3-((1-methyl- |
| | 1H-pyrazol-3-yl)amino)pyrazine-2-carboxamide |
| D-130 | 5-((2R,3R)-3-(4-cyclopropyl-2-fluorobenzamido)-2-methylpiperidin-1-yl)-3-((4-((4- |
| | methylpiperazin-1-yl)sulfonyl)phenyl)amino)pyrazine-2-carboxamide |
| D-131 | 5-((2R,3R)-2-methyl-3-(5-(p-tolyl)-1H-imidazol-2-yl)piperidin-1-yl)-3-((3- |
| | methylisothiazol-5-yl)amino)pyrazine-2-carboxamide |
| D-132 | 5-((2R,3R)-3-(4-cyclopropyl-2-fluorobenzamido)-2-methylpiperidin-1-yl)-3-((5-((4- |
| | methylpiperazin-1-yl)methyl)pyridin-2-yl)amino)pyrazine-2-carboxamide |
| D-133 | 5-((2R,3R)-3-(4-cyclopropyl-2-fluorobenzamido)-2-methylpiperidin-1-yl)-3-((4- |
| | (methylsulfonyl)phenyl)amino)pyrazine-2-carboxamide |
| D-134 | 3-((4-(1-cyclobutyl-4-methylpiperidin-4-yl)phenyl)amino)-5-((2R,3R)-3-(4-cyclopropyl- |
| | 2-fluorobenzamido)-2-methylpiperidin-1-yl)pyrazine-2-carboxamide |
| D-135 | 5-((2R,3R)-3-(4-cyclopropylbenzamido)-2-methylpiperidin-1-yl)-3-((1-methyl-1H- |
| | pyrazol-4-yl)amino)pyrazine-2-carboxamide |
| D-136 | (S)-3-((3-methylisothiazol-5-yl)amino)-5-(3-(5-(p-tolyl)-1H-imidazol-2-yl)piperidin-1- |
| | yl)pyrazine-2-carboxamide |
| D-137 | (R)-3-((3-methylisothiazol-5-yl)amino)-5-(3-(5-(p-tolyl)-1H-imidazol-2-yl)piperidin-1- |
| | yl)pyrazine-2-carboxamide |
| D-138 | 5-((2R,3R)-3-(4-cyclopropyl-2-fluorobenzamido)-2-methylpiperidin-1-yl)-3-((4-(1- |
| | cyclopropyl-4-methylpiperidin-4-yl)phenyl)amino)pyrazine-2-carboxamide |
| D-139 | 5-((2R,3R)-3-(4-cyclopropyl-2-fluorobenzamido)-2-methylpiperidin-1-yl)-3-((4- |
| | (pyrrolidin-1-ylsulfonyl)phenyl)amino)pyrazine-2-carboxamide |
| D-140 | 5-((2R,3R)-3-(4-cyclopropylbenzamido)-2-methylpiperidin-1-yl)-3-((3-(piperidin-1- |
| | ylmethyl)isothiazol-5-yl)amino)pyrazine-2-carboxamide |
| D-141 | (R)-5-(3-(6-cyclopropyl-1-oxoisoquinolin-2(1H)-yl)piperidin-1-yl)-3-((1-(1- |
| | methylpiperidin-4-yl)-1H-pyrazol-4-yl)amino)pyrazine-2-carboxamide |
| D-142 | 5-((2S,5R)-5-(4-cyclopropylbenzamido)-2-methylpiperidin-1-yl)-3-((1-methyl-1H- |
| | pyrazol-4-yl)amino)pyrazine-2-carboxamide |
| D-143 | 5-((2R,5R)-5-(4-cyclopropylbenzamido)-2-methylpiperidin-1-yl)-3-((1-methyl-1H- |
| | pyrazol-4-yl)amino)pyrazine-2-carboxamide |
| D-144 | 5-((2S,5S)-5-(4-cyclopropylbenzamido)-2-methylpiperidin-1-yl)-3-((1-methyl-1H- |
| | pyrazol-4-yl)amino)pyrazine-2-carboxamide |
| D-145 | 5-((2R,5S)-5-(4-cyclopropylbenzamido)-2-methylpiperidin-1-yl)-3-((1-methyl-1H- |
| | pyrazol-4-yl)amino)pyrazine-2-carboxamide |
| D-146 | 5-((2R,3R)-3-(4-cyclopropylbenzamido)-2-methylpiperidin-1-yl)-3-((3-fluoro-4-(4- |
| | methylpiperazin-1-yl)phenyl)amino)pyrazine-2-carboxamide |
| D-147 | 5-(3-(4-cyclopropylphenyl)-2-oxo-1-oxa-3,7-diazaspiro[4.5]decan-7-yl)-3-((1-methyl- |
| | 1H-pyrazol-4-yl)amino)pyrazine-2-carboxamide |
| D-148 | 3-((1-methyl-1H-pyrazol-4-yl)amino)-5-(3-(5-(m-tolyl)-1H-imidazol-2-yl)piperidin-1- |
| | yl)pyrazine-2-carboxamide |
| D-149 | 5-((2R,5S)-5-(4-cyclopropylbenzamido)-2-methylpiperidin-1-yl)-3-((3-fluoro-4-(4- |
| | methylpiperazin-1-yl)phenyl)amino)pyrazine-2-carboxamide |
| D-150 | 5-((2S,5R)-5-(4-cyclopropylbenzamido)-2-methylpiperidin-1-yl)-3-((3-fluoro-4-(4- |
| | methylpiperazin-1-yl)phenyl)amino)pyrazine-2-carboxamide |
| D-151 | 5-((2R,5S)-5-(3,3-dimethylureido)-2-methylpiperidin-1-yl)-3-((1-methyl-1H-pyrazol-4- |
| | yl)amino)pyrazine-2-carboxamide |
| D-152 | 5-((2S,5R)-5-(3,3-dimethylureido)-2-methylpiperidin-1-yl)-3-((1-methyl-1H-pyrazol-4- |
| | yl)amino)pyrazine-2-carboxamide |
| D-153 | 5-((2S,5R)-5-(4-cyclopropylbenzamido)-2-methylpiperidin-1-yl)-3-((4-(4- |
| | methylpiperazin-1-yl)phenyl)amino)pyrazine-2-carboxamide |
| D-154 | 5-((2R,5S)-5-(4-cyclopropylbenzamido)-2-methylpiperidin-1-yl)-3-((4-(4- |
| | methylpiperazin-1-yl)phenyl)amino)pyrazine-2-carboxamide |
| D-155 | (R)-5-(3-(6-(dimethylamino)-1-oxoisoquinolin-2(1H)-yl)piperidin-1-yl)-3-((1-methyl- |
| | 1H-pyrazol-4-yl)amino)pyrazine-2-carboxamide |
| D-156 | (R)-5-(3-(6-(dimethylamino)-1-oxo-3,4-dihydroisoquinolin-2(1H)-yl)piperidin-1-yl)-3- |
| | ((1-methyl-1H-pyrazol-4-yl)amino)pyrazine-2-carboxamide |
| D-157 | 5-((2S,5R)-5-(4-(2-hydroxypropan-2-yl)benzamido)-2-methylpiperidin-1-yl)-3-((1- |
| | methyl-1H-pyrazol-4-yl)amino)pyrazine-2-carboxamide |
| D-158 | (R)-5-(3-(4-(2-hydroxypropan-2-yl)benzamido)piperidin-1-yl)-3-((3-methylisothiazol-5- |
| | yl)amino)pyrazine-2-carboxamide |
| D-159 | 5-((2R,3R)-3-(4-(2-hydroxypropan-2-yl)benzamido)-2-methylpiperidin-1-yl)-3-((3- |
| | methylisothiazol-5-yl)amino)pyrazine-2-carboxamide |
| D-160 | 5-((2R,3R)-3-(3-fluoro-4-(2-hydroxypropan-2-yl)benzamido)-2-methylpiperidin-1-yl)-3- |
| | ((1-methyl-1H-pyrazol-4-yl)amino)pyrazine-2-carboxamide |
| D-161 | 5-((2R,3R)-3-(4-(2-methoxypropan-2-yl)benzamido)-2-methylpiperidin-1-yl)-3-((1- |
| | methyl-1H-pyrazol-4-yl)amino)pyrazine-2-carboxamide |
| D-162 | 5-((2R,3R)-3-(3,3-dimethylureido)-2-methylpiperidin-1-yl)-3-((3-(tetrahydro-2H-pyran- |
| | 4-yl)isothiazol-5-yl)amino)pyrazine-2-carboxamide |
| D-163 | 3-((3-(1-cyclopentylpiperidin-4-yl)isothiazol-5-yl)amino)-5-((2R,3R)-3-(3,3- |
| | dimethylureido)-2-methylpiperidin-1-yl)pyrazine-2-carboxamide |
| D-164 | 3-((3-methylisothiazol-5-yl)amino)-5-(3-(5-phenyl-1H-imidazol-2-yl)piperidin-1- |
| | yl)pyrazine-2-carboxamide |
| D-165 | (R)-5-(3-(6-cyclopropyl-1-oxoisoquinolin-2(1H)-yl)piperidin-1-yl)-3-((4-(4- |
| | methylpiperazin-1-yl)phenyl)amino)pyrazine-2-carboxamide |
| D-166 | (R)-5-(3-(6-cyclopropyl-1-oxoisoquinolin-2(1H)-yl)piperidin-1-yl)-3-((3- |
| | methylisothiazol-5-yl)amino)pyrazine-2-carboxamide |
| D-167 | (R)-5-(3-(6-cyclopropyl-1-oxoisoquinolin-2(1H)-yl)piperidin-1-yl)-3-((4-((4- |
| | methylpiperazin-1-yl)methyl)phenyl)amino)pyrazine-2-carboxamide |
| D-168 | (R)-5-(3-(6-cyclopropyl-1-oxoisoquinolin-2(1H)-yl)piperidin-1-yl)-3-(quinolin-6- |
| | ylamino)pyrazine-2-carboxamide |
| D-169 | (R)-5-(3-(6-cyclopropyl-1-oxoisoquinolin-2(1H)-yl)piperidin-1-yl)-3-((4-(4- |
| | methylpiperazine-1-carbonyl)phenyl)amino)pyrazine-2-carboxamide |
| D-170 | 5-((2R,3R)-3-(2-fluoro-4-((E)-prop-1-en-1-yl)benzamido)-2-methylpiperidin-1-yl)-3-((3- |
| | methylisothiazol-5-yl)amino)picolinamide |
| D-171 | (S)-3-((3-methylisothiazol-5-yl)amino)-5-(3-(5-phenyl-1H-imidazol-2-yl)piperidin-1- |
| | yl)pyrazine-2-carboxamide |
| D-172 | (R)-3-((3-methylisothiazol-5-yl)amino)-5-(3-(5-phenyl-1H-imidazol-2-yl)piperidin-1- |
| | yl)pyrazine-2-carboxamide |
| D-173 | 5-((2R,3R)-3-(4-cyclopropyl-2-fluorobenzamido)-2-methylpiperidin-1-yl)-3-((3- |
| | (morpholinomethyl)isothiazol-5-yl)amino)pyrazine-2-carboxamide |
| D-174 | (R)-5-(3-(6-cyclopropyl-8-fluoro-1-oxoisoquinolin-2(1H)-yl)piperidin-1-yl)-3-((4-(4- |
| | methylpiperazin-1-yl)phenyl)amino)pyrazine-2-carboxamide |
| D-175 | 5-((2R,3R)-3-(3,3-dimethylureido)-2-methylpiperidin-1-yl)-3-((3-methylisothiazol-5- |
| | yl)amino)picolinamide |
| D-176 | 5-((2R,3R)-3-(4-cyclopropyl-2-fluorobenzamido)-2-methylpiperidin-1-yl)-3-((3- |
| | (piperidin-1-ylmethyl)isothiazol-5-yl)amino)pyrazine-2-carboxamide |
| D-177 | (R)-3-((4-(1-cyclopentyl-4-methylpiperidin-4-yl)phenyl)amino)-5-(3-(6-cyclopropyl-1- |
| | oxoisoquinolin-2(1H)-yl)piperidin-1-yl)pyrazine-2-carboxamide |
| D-178 | (R)-5-(3-(6-cyclopropyl-1-oxoisoquinolin-2(1H)-yl)piperidin-1-yl)-3-((4-(4- |
| | methylpiperazin-1-yl)phenyl)amino)picolinamide |
| D-179 | (R)-5-(3-(6-cyclopropyl-1-oxoisoquinolin-2(1H)-yl)piperidin-1-yl)-3-((5-(4- |
| | methylpiperazin-1-yl)pyridin-2-yl)amino)pyrazine-2-carboxamide |
| D-180 | 5-((2R,3R)-3-(3,3-dimethylureido)-2-methylpiperidin-1-yl)-3-((5-(4-methylpiperazin-1- |
| | yl)pyridin-2-yl)amino)pyrazine-2-carboxamide |
| D-181 | 5-((2S,5R)-5-(4-cyclopropyl-2-fluorobenzamido)-2-methylpiperidin-1-yl)-3-((3- |
| | methylisothiazol-5-yl)amino)pyrazine-2-carboxamide |
| D-182 | 3-((4-(4-cyclopentylpiperazin-1-yl)phenyl)amino)-5-((2R,3R)-3-(3,3-dimethylureido)-2- |
| | methylpiperidin-1-yl)pyrazine-2-carboxamide |
| D-183 | 3-((4-(1-cyclopentyl-4-methylpiperidin-4-yl)phenyl)amino)-5-((2R,3R)-3-(4- |
| | (dimethylamino)benzamido)-2-methylpiperidin-1-yl)pyrazine-2-carboxamide |
| D-184 | (R)-5-(3-(6-cyclopropyl-1-oxoisoquinolin-2(1H)-yl)piperidin-1-yl)-3-((1-methyl-1H- |
| | pyrazol-4-yl)amino)pyrazine-2-carboxamide |
| D-185 | 5-((2R,3R)-3-(4-cyclopropylbenzamido)-2-methylpiperidin-1-yl)-3-((1-(1- |
| | methylpiperidin-4-yl)-1H-pyrazol-4-yl)amino)pyrazine-2-carboxamide |
| D-186 | 5-((2R,3R)-3-(5-(4-cyclopropylphenyl)-1H-imidazol-2-yl)-2-methylpiperidin-1-yl)-3- |
| | ((3-methylisothiazol-5-yl)amino)pyrazine-2-carboxamide |
| D-187 | 5-((2R,3R)-3-(4-cyclopropylbenzamido)-2-methylpiperidin-1-yl)-3-((1-(2- |
| | (dimethylamino)ethyl)-1H-pyrazol-4-yl)amino)pyrazine-2-carboxamide |
| D-188 | 5-((2R,3R)-3-(5-cyclopropylpicolinamido)-2-methylpiperidin-1-yl)-3-((4-(4- |
| | methylpiperazin-1-yl)phenyl)amino)pyrazine-2-carboxamide |
| D-189 | (S)-5-(5-(4-cyclopropylbenzamido)-3,3-difluoropiperidin-1-yl)-3-((1-methyl-1H- |
| | pyrazol-4-yl)amino)pyrazine-2-carboxamide |
| D-190 | (R)-5-(5-(4-cyclopropylbenzamido)-3,3-difluoropiperidin-1-yl)-3-((1-methyl-1H- |
| | pyrazol-4-yl)amino)pyrazine-2-carboxamide |
| D-191 | (R)-5-(3-(4-cyclopropylbenzamido)piperidin-1-yl)-3-((1-methyl-1H-pyrazol-4- |
| | yl)amino)pyrazine-2-carboxamide |
| D-192 | (R)-5-(3-(4-cyclopropylbenzamido)-4,4-difluoropiperidin-1-yl)-3-((1-methyl-1H- |
| | pyrazol-4-yl)amino)pyrazine-2-carboxamide |
| D-193 | (S)-5-(3-(4-cyclopropylbenzamido)-4,4-difluoropiperidin-1-yl)-3-((1-methyl-1H- |
| | pyrazol-4-yl)amino)pyrazine-2-carboxamide |
| D-194 | 5-((2R,3R)-3-(4-(2-hydroxypropan-2-yl)benzamido)-2-methylpiperidin-1-yl)-3-((1- |
| | methyl-1H-pyrazol-4-yl)amino)pyrazine-2-carboxamide |
| D-195 | (R)-3-(3-(4-cyclopropylbenzamido)piperidin-1-yl)-5-((1-methyl-1H-pyrazol-4- |
| | yl)amino)-1,2,4-triazine-6-carboxamide |
| D-196 | 5-((2R,3R)-3-(4-(dimethylamino)benzamido)-2-methylpiperidin-1-yl)-3-((4-(4- |
| | methylpiperazin-1-yl)phenyl)amino)pyrazine-2-carboxamide |
| D-197 | 5-((2R,3R)-3-(4-isopropylbenzamido)-2-methylpiperidin-1-yl)-3-((1-methyl-1H-pyrazol- |
| | 4-yl)amino)pyrazine-2-carboxamide |
| D-198 | N-((2R,3R)-1-(5-carbamoyl-6-((1-methyl-1H-pyrazol-4-yl)amino)pyrazin-2-yl)-2- |
| | methylpiperidin-3-yl)-5-cyclopropylpyrimidine-2-carboxamide |
| D-199 | 5-((1R,2S,5R)-2-(4-cyclopropylbenzamido)-8-azabicyclo[3.2.1]octan-8-yl)-3-((1- |
| | methyl-1H-pyrazol-4-yl)amino)pyrazine-2-carboxamide |
| D-200 | 5-((1S,2R,5S)-2-(4-cyclopropylbenzamido)-8-azabicyclo[3.2.1]octan-8-yl)-3-((1- |
| | methyl-1H-pyrazol-4-yl)amino)pyrazine-2-carboxamide |
| D-201 | 5-((1R,2R,5R)-2-(4-cyclopropylbenzamido)-8-azabicyclo[3.2.1]octan-8-yl)-3-((1- |
| | methyl-1H-pyrazol-4-yl)amino)pyrazine-2-carboxamide |
| D-202 | 5-((1S,2S,5S)-2-(4-cyclopropylbenzamido)-8-azabicyclo[3.2.1]octan-8-yl)-3-((1-methyl- |
| | 1H-pyrazol-4-yl)amino)pyrazine-2-carboxamide |
| D-203 | 5-((2R,3R)-3-(6-cyclopropylnicotinamido)-2-methylpiperidin-1-yl)-3-((4-(4- |
| | methylpiperazin-1-yl)phenyl)amino)pyrazine-2-carboxamide |
| D-205 | N-((2R,3R)-1-(5-carbamoyl-6-((1-methyl-1H-pyrazol-4-yl)amino)pyrazin-2-yl)-2- |
| | methylpiperidin-3-yl)-5-cyclopropylpyrazine-2-carboxamide |
| D-206 | (S)-3-(3-(6-cyclopropyl-1-oxoisoquinolin-2(1H)-yl)piperidin-1-yl)-5-((1-methyl-1H- |
| | pyrazol-4-yl)amino)-1,2,4-triazine-6-carboxamide |
| D-207 | 5-((2R,3R)-3-(4-cyclopropylbenzamido)-2-methylpiperidin-1-yl)-3-((1-methyl-1H- |
| | pyrazol-4-yl)amino)picolinamide |
| D-208 | 5-((2R,3R)-3-(5-(tert-butyl)picolinamido)-2-methylpiperidin-1-yl)-3-((1-methyl-1H- |
| | pyrazol-4-yl)amino)pyrazine-2-carboxamide |
| D-209 | (R)-3-((1-methyl-1H-pyrazol-4-yl)amino)-5-(3-(3-methyl-2-oxoimidazolidin-1- |
| | yl)piperidin-1-yl)pyrazine-2-carboxamide |
| D-210 | 5-((2R,3R)-3-(4-cyclopropylbenzamido)-2-methylpiperidin-1-yl)-3-((2-methylthiazol-5- |
| | yl)amino)pyrazine-2-carboxamide |
| D-211 | 5-((2R,3R)-3-(4-cyclopropylbenzamido)-2-methylpiperidin-1-yl)-3-((4- |
| | ((octahydroindolizin-7-yl)oxy)phenyl)amino)pyrazine-2-carboxamide |
| D-212 | 5-((2R,3R)-3-(4-(1-hydroxycyclopentyl)benzamido)-2-methylpiperidin-1-yl)-3-((1- |
| | methyl-1H-pyrazol-4-yl)amino)pyrazine-2-carboxamide |
| D-213 | 3-((1-methyl-1H-pyrazol-4-yl)amino)-5-((2R,3R)-2-methyl-3-(4-(1,1,1-trifluoro-2- |
| | hydroxypropan-2-yl)benzamido)piperidin-1-yl)pyrazine-2-carboxamide |
| D-214 | 3-((1-methyl-1H-pyrazol-4-yl)amino)-5-((2R,3R)-2-methyl-3-(4-(1,1,1-trifluoro-2- |
| | hydroxypropan-2-yl)benzamido)piperidin-1-yl)pyrazine-2-carboxamide |
| D-215 | 5-((2R,3R)-3-(4-cyclopropylbenzamido)-2-methylpiperidin-1-yl)-3-((1-(2-hydroxy-2- |
| | methylpropyl)-1H-pyrazol-4-yl)amino)pyrazine-2-carboxamide |
| D-216 | 5-((2R,3R)-3-(2,2-difluorobenzo[d][1,3]dioxole-5-carboxamido)-2-methylpiperidin-1- |
| | yl)-3-((1-methyl-1H-pyrazol-4-yl)amino)pyrazine-2-carboxamide |
| D-217 | 5-((2R,3R)-3-(4-((2-hydroxyethyl)(methyl)amino)benzamido)-2-methylpiperidin-1-yl)- |
| | 3-((1-methyl-1H-pyrazol-4-yl)amino)pyrazine-2-carboxamide |
| D-218 | 5-((2R,3R)-3-(4-(diethylamino)benzamido)-2-methylpiperidin-1-yl)-3-((1-methyl-1H- |
| | pyrazol-4-yl)amino)pyrazine-2-carboxamide |
| D-219 | 5-((2R,3R)-3-(4-cyclopropoxybenzamido)-2-methylpiperidin-1-yl)-3-((1-methyl-1H- |
| | pyrazol-4-yl)amino)pyrazine-2-carboxamide |
| D-220 | 5-((2R,3R)-3-((4-isopropylphenyl)sulfonamido)-2-methylpiperidin-1-yl)-3-((1-methyl- |
| | 1H-pyrazol-4-yl)amino)pyrazine-2-carboxamide |
| D-221 | 3-((1-methyl-1H-pyrazol-4-yl)amino)-5-((2R,3R)-2-methyl-3-(4-(1- |
| | (trifluoromethyl)cyclopropyl)benzamido)piperidin-1-yl)pyrazine-2-carboxamide |
| D-222 | (R)-5-(3-(4-isopropylbenzamido)piperidin-1-yl)-3-((1-methyl-1H-pyrazol-4- |
| | yl)amino)pyrazine-2-carboxamide |
| D-223 | 5-((2R,3R)-3-(4-(tert-butyl)-2-fluorobenzamido)-2-methylpiperidin-1-yl)-3-((1-methyl- |
| | 1H-pyrazol-4-yl)amino)pyrazine-2-carboxamide |
| D-224 | 5-((2R,3R)-3-(2,3-dihydrobenzo[b][1,4]dioxine-6-carboxamido)-2-methylpiperidin-1- |
| | yl)-3-((1-methyl-1H-pyrazol-4-yl)amino)pyrazine-2-carboxamide |
| D-225 | (R)-5-(3-(6-cyclopropyl-1-oxo-2,7-naphthyridin-2(1H)-yl)piperidin-1-yl)-3-((1-methyl- |
| | 1H-pyrazol-4-yl)amino)pyrazine-2-carboxamide |
| D-226 | N-((2R,3R)-1-(5-carbamoyl-6-((4-(4-methylpiperazin-1-yl)phenyl)amino)pyrazin-2-yl)- |
| | 2-methylpiperidin-3-yl)benzo[d]thiazole-5-carboxamide |
| D-227 | 5-((2R,3R)-2-methyl-3-(4-(methylsulfonyl)benzamido)piperidin-1-yl)-3-((4-(4- |
| | methylpiperazin-1-yl)phenyl)amino)pyrazine-2-carboxamide |
| D-228 | 3-((1-methyl-1H-pyrazol-4-yl)amino)-5-((2R,3R)-2-methyl-3-(4-(3-methyloxetan-3- |
| | yl)benzamido)piperidin-1-yl)pyrazine-2-carboxamide |
| D-229 | 5-((2R,3R)-3-(5-(tert-butyl)thiophene-2-carboxamido)-2-methylpiperidin-1-yl)-3-((1- |
| | methyl-1H-pyrazol-4-yl)amino)pyrazine-2-carboxamide |
| D-230 | 3-((1-methyl-1H-pyrazol-4-yl)amino)-5-((2R,3R)-2-methyl-3-(4-(pentafluoro-16- |
| | sulfanyl)benzamido)piperidin-1-yl)pyrazine-2-carboxamide |
| D-231 | 5-((2R,3R)-3-(4-cyclopropyl-3-methoxybenzamido)-2-methylpiperidin-1-yl)-3-((1- |
| | methyl-1H-pyrazol-4-yl)amino)pyrazine-2-carboxamide |
| D-232 | 3-((1-methyl-1H-pyrazol-4-yl)amino)-5-((2R,3R)-2-methyl-3-(4- |
| | methylbenzamido)piperidin-1-yl)pyrazine-2-carboxamide |
| D-233 | 5-((2R,3R)-2-methyl-3-(4-((trifluoromethyl)thio)benzamido)piperidin-1-yl)-3-((4-(4- |
| | methylpiperazin-1-yl)phenyl)amino)pyrazine-2-carboxamide |
| D-234 | N-((2R,3R)-1-(5-carbamoyl-6-((4-(4-methylpiperazin-1-yl)phenyl)amino)pyrazin-2-yl)- |
| | 2-methylpiperidin-3-yl)-1-methyl-1H-indazole-5-carboxamide |
| D-235 | 5-((2R,3R)-3-(4-(1-hydroxycyclopropyl)benzamido)-2-methylpiperidin-1-yl)-3-((1- |
| | methyl-1H-pyrazol-4-yl)amino)pyrazine-2-carboxamide |
| D-236 | 5-((2S,5R)-5-(4-(2-hydroxypropan-2-yl)benzamido)-2-methylpiperidin-1-yl)-3-((1- |
| | methyl-1H-pyrazol-4-yl)amino)pyrazine-2-carboxamide |
| D-237 | N-((2R,3R)-1-(5-carbamoyl-6-((1-methyl-1H-pyrazol-4-yl)amino)pyrazin-2-yl)-2- |
| | methylpiperidin-3-yl)benzo[d]thiazole-5-carboxamide |
| D-238 | N-((2R,3R)-1-(5-carbamoyl-6-((1-methyl-1H-pyrazol-4-yl)amino)pyrazin-2-yl)-2- |
| | methylpiperidin-3-yl)-2-methylbenzo[d]oxazole-5-carboxamide |
| D-239 | 2-(tert-butyl)-N-((2R,3R)-1-(5-carbamoyl-6-((1-methyl-1H-pyrazol-4-yl)amino)pyrazin- |
| | 2-yl)-2-methylpiperidin-3-yl)thiazole-5-carboxamide |
| D-240 | 5-((2R,3R)-3-(4-(1,1,1,3,3,3-hexafluoro-2-hydroxypropan-2-yl)benzamido)-2- |
| | methylpiperidin-1-yl)-3-((1-methyl-1H-pyrazol-4-yl)amino)pyrazine-2-carboxamide |
| D-241 | 3-((1-methyl-1H-pyrazol-4-yl)amino)-5-((2R,3R)-2-methyl-3-(4- |
| | ((trifluoromethyl)thio)benzamido)piperidin-1-yl)pyrazine-2-carboxamide |
| D-242 | N-((2R,3R)-1-(5-carbamoyl-6-((1-methyl-1H-pyrazol-4-yl)amino)pyrazin-2-yl)-2- |
| | methylpiperidin-3-yl)-2-methylbenzo[d]thiazole-5-carboxamide |
| D-243 | N-((2R,3R)-1-(5-carbamoyl-6-((1-methyl-1H-pyrazol-4-yl)amino)pyrazin-2-yl)-2- |
| | methylpiperidin-3-yl)benzo[d]thiazole-6-carboxamide |
| D-244 | 5-((2R,3R)-3-(3-(tert-butyl)-1H-pyrazole-5-carboxamido)-2-methylpiperidin-1-yl)-3-((1- |
| | methyl-1H-pyrazol-4-yl)amino)pyrazine-2-carboxamide |
| D-245 | 5-((2R,3R)-3-(4-cyclopropyl-3-hydroxybenzamido)-2-methylpiperidin-1-yl)-3-((1- |
| | methyl-1H-pyrazol-4-yl)amino)pyrazine-2-carboxamide |
| D-246 | 3-((1-methyl-1H-pyrazol-4-yl)amino)-5-((2R,3R)-2-methyl-3-(2-methylbenzofuran-5- |
| | carboxamido)piperidin-1-yl)pyrazine-2-carboxamide |
| D-247 | 5-(tert-butyl)-N-((2R,3R)-1-(5-carbamoyl-6-((1-methyl-1H-pyrazol-4-yl)amino)pyrazin- |
| | 2-yl)-2-methylpiperidin-3-yl)isoxazole-3-carboxamide |
| D-248 | 5-((2R,3R)-3-(4-((2-methoxyethyl)(methyl)amino)benzamido)-2-methylpiperidin-1-yl)- |
| | 3-((1-methyl-1H-pyrazol-4-yl)amino)pyrazine-2-carboxamide |
| D-249 | 3-((1-methyl-1H-pyrazol-4-yl)amino)-5-((2R,3R)-2-methyl-3-(4-((R)-2,2,2-trifluoro-1- |
| | hydroxyethyl)benzamido)piperidin-1-yl)pyrazine-2-carboxamide |
| D-250 | 3-((1-methyl-1H-pyrazol-4-yl)amino)-5-((2R,3R)-2-methyl-3-(4-((S)-2,2,2-trifluoro-1- |
| | hydroxyethyl)benzamido)piperidin-1-yl)pyrazine-2-carboxamide |
| D-251 | 5-((2R,3R)-3-(4-(2-fluoropropan-2-yl)benzamido)-2-methylpiperidin-1-yl)-3-((1-methyl- |
| | 1H-pyrazol-4-yl)amino)pyrazine-2-carboxamide |
| D-252 | 5-((2R,3R)-3-(3-cyclopropyl-1H-pyrazole-5-carboxamido)-2-methylpiperidin-1-yl)-3- |
| | ((1-methyl-1H-pyrazol-4-yl)amino)pyrazine-2-carboxamide |
| D-253 | 5-((2R,3R)-3-(5-cyclopropyl-1-methyl-1H-pyrazole-3-carboxamido)-2-methylpiperidin- |
| | 1-yl)-3-((1-methyl-1H-pyrazol-4-yl)amino)pyrazine-2-carboxamide |
| D-254 | N-((2R,3R)-1-(5-carbamoyl-6-((1-methyl-1H-pyrazol-4-yl)amino)pyrazin-2-yl)-2- |
| | methylpiperidin-3-yl)-1-methyl-1H-indazole-5-carboxamide |
| D-255 | N-((2R,3R)-1-(5-carbamoyl-6-((1-methyl-1H-pyrazol-4-yl)amino)pyrazin-2-yl)-2- |
| | methylpiperidin-3-yl)-1-isopropyl-1H-benzo[d][1,2,3]triazole-5-carboxamide |
| D-256 | 5-((2R,3R)-3-(4-((R)-1,2-dihydroxypropan-2-yl)benzamido)-2-methylpiperidin-1-yl)-3- |
| | ((1-methyl-1H-pyrazol-4-yl)amino)pyrazine-2-carboxamide |
| D-257 | N-((2R,3R)-1-(5-carbamoyl-6-((1-methyl-1H-pyrazol-4-yl)amino)pyrazin-2-yl)-2- |
| | methylpiperidin-3-yl)-2-cyclopropyloxazole-4-carboxamide |
| D-258 | 5-((2R,3R)-3-(2,2-dimethylchromane-6-carboxamido)-2-methylpiperidin-1-yl)-3-((1- |
| | methyl-1H-pyrazol-4-yl)amino)pyrazine-2-carboxamide |
| D-259 | N-((2R,3R)-1-(5-carbamoyl-6-((1-methyl-1H-pyrazol-4-yl)amino)pyrazin-2-yl)-2- |
| | methylpiperidin-3-yl)-1-methyl-1H-indazole-6-carboxamide |
| D-260 | 5-((2R,3R)-3-(4-(1-(hydroxymethyl)cyclopropyl)benzamido)-2-methylpiperidin-1-yl)-3- |
| | ((1-methyl-1H-pyrazol-4-yl)amino)pyrazine-2-carboxamide |
| D-261 | N-((2R,3R)-1-(5-carbamoyl-6-((1-methyl-1H-pyrazol-4-yl)amino)pyrazin-2-yl)-2- |
| | methylpiperidin-3-yl)-2-methyl-1H-benzo[d]imidazole-5-carboxamide |
| D-262 | N-((2R,3R)-1-(5-carbamoyl-6-((1-methyl-1H-pyrazol-4-yl)amino)pyrazin-2-yl)-2- |
| | methylpiperidin-3-yl)-1H-benzo[d]imidazole-5-carboxamide |
| D-263 | N-((2R,3R)-1-(5-carbamoyl-6-((1-methyl-1H-pyrazol-4-yl)amino)pyrazin-2-yl)-2- |
| | methylpiperidin-3-yl)-5,6-dihydro-4H-pyrrolo[1,2-b]pyrazole-2-carboxamide |
| D-264 | 5-((2R,3R)-3-(2-cyclopropyl-1H-imidazole-4-carboxamido)-2-methylpiperidin-1-yl)-3- |
| | ((1-methyl-1H-pyrazol-4-yl)amino)pyrazine-2-carboxamide |
| D-265 | 5-((2R,3R)-3-(4-((S)-1,2-dihydroxypropan-2-yl)benzamido)-2-methylpiperidin-1-yl)-3- |
| | ((1-methyl-1H-pyrazol-4-yl)amino)pyrazine-2-carboxamide |
| D-266 | N-((2R,3R)-1-(5-carbamoyl-6-((1-methyl-1H-pyrazol-4-yl)amino)pyrazin-2-yl)-2- |
| | methylpiperidin-3-yl)imidazo[1,2-a]pyridine-6-carboxamide |
| D-267 | N-((2R,3R)-1-(5-carbamoyl-6-((1-methyl-1H-pyrazol-4-yl)amino)pyrazin-2-yl)-2- |
| | methylpiperidin-3-yl)-2-methyl-2H-indazole-5-carboxamide |
| D-268 | N-((2R,3R)-1-(5-carbamoyl-6-((1-methyl-1H-pyrazol-4-yl)amino)pyrazin-2-yl)-2- |
| | methylpiperidin-3-yl)terephthalamide |
| D-269 | 5-((2R,3R)-3-(4-cyclopropylbenzamido)-2-methylpiperidin-1-yl)-3-((3-(((2- |
| | methoxyethyl)(methyl)amino)methyl)isothiazol-5-yl)amino)pyrazine-2-carboxamide |
| D-270 | 5-((2R,3R)-3-(4-cyclopropylbenzamido)-2-methylpiperidin-1-yl)-3-((1-(2- |
| | methoxyethyl)-1H-pyrazol-4-yl)amino)pyrazine-2-carboxamide |
| D-271 | 5-((2R,3R)-3-(4-cyclopropylbenzamido)-2-methylpiperidin-1-yl)-3-((1,5-dimethyl-1H- |
| | pyrazol-4-yl)amino)pyrazine-2-carboxamide |
| D-272 | 5-((2R,3R)-3-(4-cyclopropylbenzamido)-2-methylpiperidin-1-yl)-3-((1,3-dimethyl-1H- |
| | pyrazol-4-yl)amino)pyrazine-2-carboxamide |
| D-273 | 5-((2R,3R)-3-(4-cyclopropylbenzamido)-2-methylpiperidin-1-yl)-3-((1-ethyl-1H- |
| | pyrazol-4-yl)amino)pyrazine-2-carboxamide |
| D-274 | 5-((2R,3R)-3-(4-cyclopropylbenzamido)-2-methylpiperidin-1-yl)-3-((1-(tetrahydro-2H- |
| | pyran-4-yl)-1H-pyrazol-4-yl)amino)pyrazine-2-carboxamide |
| D-275 | 5-((2R,3R)-3-(4-cyclopropylbenzamido)-2-methylpiperidin-1-yl)-3-((1-(difluoromethyl)- |
| | 1H-pyrazol-4-yl)amino)pyrazine-2-carboxamide |
| D-276 | 5-((2R,3R)-3-(4-cyclopropylbenzamido)-2-methylpiperidin-1-yl)-3-((1-methyl-5- |
| | (trifluoromethyl)-1H-pyrazol-4-yl)amino)pyrazine-2-carboxamide |
| D-277 | 3-((2R,3R)-3-(4-cyclopropylbenzamido)-2-methylpiperidin-1-yl)-5-((1-methyl-1H- |
| | pyrazol-4-yl)amino)-1,2,4-triazine-6-carboxamide |
| D-278 | 5-((2R,3R)-3-(4-cyclopropylbenzamido)-2-methylpiperidin-1-yl)-3-((1-methyl-3- |
| | (trifluoromethyl)-1H-pyrazol-4-yl)amino)pyrazine-2-carboxamide |
| D-279 | 3-((1-cyclopropyl-1H-pyrazol-4-yl)amino)-5-((2R,3R)-3-(4-cyclopropylbenzamido)-2- |
| | methylpiperidin-1-yl)pyrazine-2-carboxamide |
| D-280 | 5-((2R,3R)-3-(4-cyclopropylbenzamido)-2-methylpiperidin-1-yl)-3-((1-(piperidin-4-yl)- |
| | 1H-pyrazol-4-yl)amino)pyrazine-2-carboxamide |
| D-281 | 5-((2R,3R)-3-(4-cyclopropylbenzamido)-2-methylpiperidin-1-yl)-3-((1-(2- |
| | hydroxyethyl)-1H-pyrazol-4-yl)amino)pyrazine-2-carboxamide |
| D-282 | 3-((1H-pyrazol-4-yl)amino)-5-((2R,3R)-3-(4-cyclopropylbenzamido)-2-methylpiperidin- |
| | 1-yl)pyrazine-2-carboxamide |
| D-283 | 3-((1-(1-acetylpiperidin-4-yl)-1H-pyrazol-4-yl)amino)-5-((2R,3R)-3-(4- |
| | cyclopropylbenzamido)-2-methylpiperidin-1-yl)pyrazine-2-carboxamide |
| D-284 | 5-((2R,3R)-3-(4-cyclopropylbenzamido)-2-methylpiperidin-1-yl)-3-((1-(pyridin-4-yl)- |
| | 1H-pyrazol-4-yl)amino)pyrazine-2-carboxamide |
| D-285 | 3-((2R,3R)-3-(4-(tert-butyl)benzamido)-2-methylpiperidin-1-yl)-5-((1-methyl-1H- |
| | pyrazol-4-yl)amino)-1,2,4-triazine-6-carboxamide |
| D-286 | 3-((2R,3R)-3-(3,3-dimethylureido)-2-methylpiperidin-1-yl)-5-((1-methyl-1H-pyrazol-4- |
| | yl)amino)-1,2,4-triazine-6-carboxamide |
| D-287 | 3-((1-(cyanomethyl)-1H-pyrazol-4-yl)amino)-5-((2R,3R)-3-(4-cyclopropylbenzamido)- |
| | 2-methylpiperidin-1-yl)pyrazine-2-carboxamide |
| D-288 | 3-((1-(1-(cyclopropanecarbonyl)piperidin-4-yl)-1H-pyrazol-4-yl)amino)-5-((2R,3R)-3- |
| | (4-cyclopropylbenzamido)-2-methylpiperidin-1-yl)pyrazine-2-carboxamide |
| D-289 | 5-((2R,3R)-3-(4-cyclopropylbenzamido)-2-methylpiperidin-1-yl)-3-((1-(1-(3- |
| | methyloxetane-3-carbonyl)piperidin-4-yl)-1H-pyrazol-4-yl)amino)pyrazine-2- |
| | carboxamide |
| D-290 | 5-((2R,3R)-3-(4-cyclopropylbenzamido)-2-methylpiperidin-1-yl)-3-((5-fluoro-1-methyl- |
| | 1H-pyrazol-4-yl)amino)pyrazine-2-carboxamide |
| D-291 | 5-((2R,3R)-3-(4-(tert-butyl)benzamido)-2-methylpiperidin-1-yl)-3-((4-((1- |
| | methylpiperidin-4-yl)oxy)phenyl)amino)pyrazine-2-carboxamide |
| D-292 | 5-((2R,3R)-3-(4-(2-cyanopropan-2-yl)benzamido)-2-methylpiperidin-1-yl)-3-((4-((1- |
| | methylpiperidin-4-yl)oxy)phenyl)amino)pyrazine-2-carboxamide |
| D-293 | 5-((2R,3R)-3-(4-cyclopropylbenzamido)-2-methylpiperidin-1-yl)-3-((1-(1-(2,2,2- |
| | trifluoroethyl)piperidin-4-yl)-1H-pyrazol-4-yl)amino)pyrazine-2-carboxamide |
| D-294 | 5-((2R,3R)-3-(6-cyclopropylnicotinamido)-2-methylpiperidin-1-yl)-3-((4-((1- |
| | methylpiperidin-4-yl)oxy)phenyl)amino)pyrazine-2-carboxamide |
| D-295 | 3-((4-(4-acetylpiperazin-1-yl)phenyl)amino)-5-((2R,3R)-3-(4-cyclopropylbenzamido)-2- |
| | methylpiperidin-1-yl)pyrazine-2-carboxamide |
| D-296 | 3-((4-(1-acetylpiperidin-4-yl)phenyl)amino)-5-((2R,3R)-3-(4-cyclopropylbenzamido)-2- |
| | methylpiperidin-1-yl)pyrazine-2-carboxamide |
| D-297 | 5-((2R,3R)-3-(5-cyclopropylpicolinamido)-2-methylpiperidin-1-yl)-3-((4-((1- |
| | methylpiperidin-4-yl)oxy)phenyl)amino)pyrazine-2-carboxamide |
| D-298 | 3-((2R,3R)-3-(4-(2-hydroxypropan-2-yl)benzamido)-2-methylpiperidin-1-yl)-5-((1- |
| | methyl-1H-pyrazol-4-yl)amino)-1,2,4-triazine-6-carboxamide |
| D-299 | 5-((2R,3R)-3-(4-cyclopropylbenzamido)-2-methylpiperidin-1-yl)-3-((4-(2- |
| | ethoxyethoxy)phenyl)amino)pyrazine-2-carboxamide |
| D-300 | 5-((2R,3R)-3-(4-cyclopropylbenzamido)-2-methylpiperidin-1-yl)-3-(((1-methyl-1,2,3,6- |
| | tetrahydropyridin-4-yl)methyl)amino)pyrazine-2-carboxamide |
| D-301 | 5-((2R,3R)-3-(4-cyclopropylbenzamido)-2-methylpiperidin-1-yl)-3-((4-(1- |
| | methylpiperidin-4-yl)phenyl)amino)pyrazine-2-carboxamide |
| D-302 | 5-((2R,3R)-3-(2-fluoro-4-(2-hydroxypropan-2-yl)benzamido)-2-methylpiperidin-1-yl)-3- |
| | ((1-methyl-1H-pyrazol-4-yl)amino)pyrazine-2-carboxamide |
| D-303 | 5-((2R,3R)-3-(4-cyclopropylbenzamido)-2-methylpiperidin-1-yl)-3-(isochroman-6- |
| | ylamino)pyrazine-2-carboxamide |
| D-304 | 5-((2R,3R)-3-(3-ethyl-3-methylureido)-2-methylpiperidin-1-yl)-3-((1-methyl-1H- |
| | pyrazol-4-yl)amino)pyrazine-2-carboxamide |
| D-305 | 5-((2R,3R)-3-(6-cyclopropylnicotinamido)-2-methylpiperidin-1-yl)-3-((4-(4,4- |
| | difluoropiperidin-1-yl)phenyl)amino)pyrazine-2-carboxamide |
| D-306 | 3-((4-(4,4-difluoropiperidin-1-yl)phenyl)amino)-5-((2R,3R)-3-(3,3-dimethylureido)-2- |
| | methylpiperidin-1-yl)pyrazine-2-carboxamide |
| D-307 | 5-((2R,3R)-3-(4-cyclopropylbenzamido)-2-methylpiperidin-1-yl)-3-((1-(4,4- |
| | difluorocyclohexyl)-1H-pyrazol-4-yl)amino)pyrazine-2-carboxamide |
| D-308 | 3-((2R,3R)-3-(2-fluoro-4-(2-hydroxypropan-2-yl)benzamido)-2-methylpiperidin-1-yl)-5- |
| | ((1-methyl-1H-pyrazol-4-yl)amino)-1,2,4-triazine-6-carboxamide |
| D-309 | 5-((2R,3R)-3-(6-cyclopropylnicotinamido)-2-methylpiperidin-1-yl)-3-((4-((tetrahydro- |
| | 2H-pyran-4-yl)oxy)phenyl)amino)pyrazine-2-carboxamide |
| D-310 | 5-((2R,3R)-3-(4-cyclopropylbenzamido)-2-methylpiperidin-1-yl)-3-((3-(tetrahydro-2H- |
| | pyran-4-yl)isothiazol-5-yl)amino)pyrazine-2-carboxamide |
| D-311 | 3-((3-(1-cyclopentylpiperidin-4-yl)isothiazol-5-yl)amino)-5-((2R,3R)-3-(4- |
| | cyclopropylbenzamido)-2-methylpiperidin-1-yl)pyrazine-2-carboxamide |
| D-312 | 5-((2R,3R)-3-(4-cyclopropylbenzamido)-2-methylpiperidin-1-yl)-3-((3-(1- |
| | methylpiperidin-4-yl)isothiazol-5-yl)amino)pyrazine-2-carboxamide |
| D-313 | 5-((2R,3R)-3-(4-cyclopropyl-2-fluorobenzamido)-2-methylpiperidin-1-yl)-3-((3-fluoro- |
| | 4-(piperazin-1-yl)phenyl)amino)pyrazine-2-carboxamide |
| D-314 | 5-((2R,3R)-3-(3-cyclopropyl-3-methylureido)-2-methylpiperidin-1-yl)-3-((1-methyl-1H- |
| | pyrazol-4-yl)amino)pyrazine-2-carboxamide |
| D-315 | 5-((2R,3R)-3-(4-cyclopropylbenzamido)-2-methylpiperidin-1-yl)-3-((2,2- |
| | difluorobenzo[d][1,3]dioxol-5-yl)amino)pyrazine-2-carboxamide |
| D-316 | 5-((2R,3R)-3-(4-cyclopropylbenzamido)-2-methylpiperidin-1-yl)-3-((4- |
| | (morpholinomethyl)phenyl)amino)pyrazine-2-carboxamide |
| D-317 | 5-((2R,3R)-3-(4-cyclopropylbenzamido)-2-methylpiperidin-1-yl)-3-((4-(tetrahydro-2H- |
| | pyran-4-yl)phenyl)amino)pyrazine-2-carboxamide |
| D-318 | 5-((2R,3R)-3-(4-cyclopropylbenzamido)-2-methylpiperidin-1-yl)-3-((2-(4- |
| | methylpiperazin-1-yl)pyrimidin-5-yl)amino)pyrazine-2-carboxamide |
| D-319 | 5-((2R,3R)-3-(4-cyclopropylbenzamido)-2-methylpiperidin-1-yl)-3-((1-((tetrahydro-2H- |
| | pyran-4-yl)methyl)-1H-pyrazol-4-yl)amino)pyrazine-2-carboxamide |
| D-320 | 5-((2R,3R)-3-(4-cyclopropylbenzamido)-2-methylpiperidin-1-yl)-3-((4-(4- |
| | (dimethylcarbamoyl)piperidin-1-yl)phenyl)amino)pyrazine-2-carboxamide |
| D-321 | 5-((2R,3R)-3-(4-cyclopropylbenzamido)-2-methylpiperidin-1-yl)-3-((1-((4,4- |
| | difluorocyclohexyl)methyl)-1H-pyrazol-4-yl)amino)pyrazine-2-carboxamide |

In another particular embodiment, the compound is selected from the group consisting of compounds listed in Table N8:

TABLE N8

| Ex. # | Chemical Name |
|---|---|
| E-1 | (R)-5-((1-acryloylpyrrolidin-3-yl)(methyl)amino)-3-((2-methylthiazol-5- |
| | yl)amino)pyrazine-2-carboxamide; |
| E-2 | tert-butyl (R)-3-((6-carbamoyl-5-((4-(oxazol-2-yl)phenyl)amino)-1,2,4-triazin-3- |
| | yl)(methyl)amino)piperidine-1-carboxylate; |
| E-5 | 3-((2R,3R)-3-acrylamido-2-methylpiperidin-1-yl)-5-((4-(1-cyclopropylpiperidin-4- |
| | yl)phenyl)amino)-1,2,4-triazine-6-carboxamide; |
| E-6 | 5-((2R,3R)-3-acrylamido-2-methylpiperidin-1-yl)-3-((4-(1-cyclopropyl-4- |
| | methylpiperidin-4-yl)phenyl)amino)pyrazine-2-carboxamide; |
| E-7 | 3-((2R,3R)-3-acrylamido-2-methylpiperidin-1-yl)-5-((4-(1-cyclopropyl-4- |
| | methylpiperidin-4-yl)phenyl)amino)-1,2,4-triazine-6-carboxamide; |
| E-10 | 3-((3R,3′S,4′S)-3-((3-chloro-5-(trifluoromethyl)phenyl)amino)-4′-hydroxy-2-oxo-[1,3′- |
| | bipiperidin]-1′-yl)-5-((3-methylisothiazol-5-yl)amino)-1,2,4-triazine-6-carboxamide; |
| E-11 | (R)-3-((1-acryloylpiperidin-3-yl)amino)-5-((3-methylisothiazol-5-yl)amino)-1,2,4- |
| | triazine-6-carboxamide; |
| E-12 | (R)-3-((1-acryloylpyrrolidin-3-yl)(methyl)amino)-5-((3-methylisothiazol-5-yl)amino)- |
| | 1,2,4-triazine-6-carboxamide; |
| E-13 | (R)-3-((1-(4-(1,1,1,3,3,3-hexafluoro-2-hydroxypropan-2-yl)benzoyl)pyrrolidin-3- |
| | yl)(methyl)amino)-5-((3-methylisothiazol-5-yl)amino)-1,2,4-triazine-6-carboxamide; |
| E-14 | (R)-5-((1-acryloylpyrrolidin-3-yl)(methyl)amino)-3-(cyclopropylamino)pyrazine-2- |
| | carboxamide; |
| E-16 | (R)-3-((1-acryloylpyrrolidin-3-yl)(methyl)amino)-5-((4-(1,1,1,3,3,3-hexafluoro-2- |
| | hydroxypropan-2-yl)phenyl)amino)-1,2,4-triazine-6-carboxamide; |
| E-17 | 3-(((2S,3R)-1-acryloyl-2-methylpiperidin-3-yl)amino)-5-((4-isopropylphenyl)amino)- |
| | 1,2,4-triazine-6-carboxamide; |
| E-18 | (S)-3-((1-acryloylpiperidin-3-yl)amino)-5-((4-(oxazol-2-yl)phenyl)amino)-1,2,4-triazine- |
| | 6-carboxamide; |
| E-19 | (S)-3-((1-acryloylazepan-3-yl)amino)-5-((4-isopropylphenyl)amino)-1,2,4-triazine-6- |
| | carboxamide; |
| E-20 | (R)-3-((1-acryloylazepan-3-yl)amino)-5-((4-isopropylphenyl)amino)-1,2,4-triazine-6- |
| | carboxamide; |
| E-21 | (R)-3-(3-acrylamidopiperidin-1-yl)-5-((4-isopropylphenyl)amino)-1,2,4-triazine-6- |
| | carboxamide; |
| E-22 | (S)-3-(3-acrylamidopiperidin-1-yl)-5-((4-isopropylphenyl)amino)-1,2,4-triazine-6- |
| | carboxamide; |
| E-23 | (S)-3-((1-acryloylpiperidin-3-yl)amino)-5-((4-(tert-butyl)phenyl)amino)-1,2,4-triazine-6- |
| | carboxamide; |
| E-24 | (R)-3-((1-acryloylpiperidin-3-yl)amino)-5-((4-(tert-butyl)phenyl)amino)-1,2,4-triazine-6- |
| | carboxamide; |
| E-25 | 3-(((1R,2S)-2-acrylamidocyclohexyl)amino)-5-((4-isopropylphenyl)amino)-1,2,4- |
| | triazine-6-carboxamide; |
| E-26 | (R)-3-((1-acryloylpyrrolidin-3-yl)(methyl)amino)-5-((4-isopropylphenyl)amino)-1,2,4- |
| | triazine-6-carboxamide; |
| E-27 | (S)-3-((1-acryloylpiperidin-3-yl)(methyl)amino)-5-((4-isopropylphenyl)amino)-1,2,4- |
| | triazine-6-carboxamide; |
| E-28 | 3-(((1R,2R)-2-acrylamidocyclohexyl)amino)-5-((4-isopropylphenyl)amino)-1,2,4- |
| | triazine-6-carboxamide; |
| E-29 | 3-(((1S,2S)-2-acrylamidocyclohexyl)amino)-5-((4-isopropylphenyl)amino)-1,2,4- |
| | triazine-6-carboxamide; |
| E-32 | (R)-5-((4-(1-acryloyl-4-methylpiperidin-4-yl)phenyl)amino)-3-((1-acryloylpiperidin-3- |
| | yl)amino)-1,2,4-triazine-6-carboxamide; |
| E-34 | 3-(((1S,2R)-2-acrylamidocyclohexyl)amino)-5-((4-isopropylphenyl)amino)-1,2,4- |
| | triazine-6-carboxamide; |
| E-35 | (R)-5-((4-(tert-butyl)phenyl)amino)-3-((1-propionylpiperidin-3-yl)amino)-1,2,4-triazine- |
| | 6-carboxamide; |
| E-36 | (R)-3-((1-acryloylpiperidin-3-yl)amino)-5-((4-cyclopropylphenyl)amino)-1,2,4-triazine- |
| | 6-carboxamide; |
| E-37 | (R)-3-(N-(1-acryloylpiperidin-3-yl)acrylamido)-5-((4-cyclopropylphenyl)amino)-1,2,4- |
| | triazine-6-carboxamide; |
| E-38 | (R)-3-((1-acryloylpiperidin-3-yl)amino)-5-((4-(1-cyanocyclopropyl)phenyl)amino)-1,2,4- |
| | triazine-6-carboxamide; |
| E-39 | (R)-3-(N-(1-acryloylpiperidin-3-yl)acrylamido)-5-((4-(1- |
| | cyanocyclopropyl)phenyl)amino)-1,2,4-triazine-6-carboxamide; |
| E-40 | (R)-3-((1-acryloylpiperidin-3-yl)amino)-5-((4-(oxazol-2-yl)phenyl)amino)-1,2,4-triazine- |
| | 6-carboxamide; |
| E-41 | (R)-3-((1-acryloylpiperidin-3-yl)amino)-5-((4-(pyrimidin-2-yl)phenyl)amino)-1,2,4- |
| | triazine-6-carboxamide; |
| E-42 | (R)-3-((1-acryloylpiperidin-3-yl)amino)-5-((4-isopropyl-3-methylphenyl)amino)-1,2,4- |
| | triazine-6-carboxamide; |
| E-43 | (R)-3-((1-acryloylpiperidin-3-yl)amino)-5-(p-tolylamino)-1,2,4-triazine-6-carboxamide; |
| E-44 | (R)-3-((1-acryloylpipendin-3-yl)amino)-5-(m-tolylamino)-1,2,4-triazine-6-carboxamide; |
| E-45 | (R)-3-((1-acryloylpiperidin-3-yl)amino)-5-((4-(1-propionylpiperidin-4-yl)phenyl)amino)- |
| | 1,2,4-triazine-6-carboxamide; |
| E-46 | (R)-3-((1-acryloylpiperidin-3-yl)amino)-5-((4-(1-cyanocyclopentyl)phenyl)amino)-1,2,4- |
| | triazine-6-carboxamide; |
| E-47 | (R)-3-((1-acryloylpiperidin-3-yl)amino)-5-((4-(methylsulfonyl)phenyl)amino)-1,2,4- |
| | triazine-6-carboxamide; |
| E-48 | (R)-3-((1-acryloylpiperidin-3-yl)amino)-5-((4-(1-cyclopentylpiperidin-4- |
| | yl)phenyl)amino)-1,2,4-triazine-6-carboxamide; |
| E-49 | (R)-3-((1-acryloylpiperidin-3-yl)amino)-5-((4-(2-cyanopropan-2-yl)phenyl)amino)-1,2,4- |
| | triazine-6-carboxamide; |
| E-50 | (R)-3-((1-acryloylpiperidin-3-yl)amino)-5-((4-iodophenyl)amino)-1,2,4-triazine-6- |
| | carboxamide; |
| E-51 | (R)-3-((1-acryloylpiperidin-3-yl)amino)-5-(benzo[d]thiazol-6-ylamino)-1,2,4-triazine-6- |
| | carboxamide; |
| E-52 | (R)-5-((4-((1H-1,2,4-triazol-1-yl)methyl)phenyl)amino)-3-((1-acryloylpiperidin-3- |
| | yl)amino)-1,2,4-triazine-6-carboxamide; |
| E-53 | (R)-3-((1-acryloylpiperidin-3-yl)amino)-5-((5-fluoropyridin-3-yl)amino)-1,2,4-triazine-6- |
| | carboxamide; |
| E-54 | (R)-3-((1-acryloylpiperidin-3-yl)amino)-5-(quinolin-3-ylamino)-1,2,4-triazine-6- |
| | carboxamide; |
| E-55 | (R)-3-((1-acryloylpiperidin-3-yl)amino)-5-((3-(pyrimidin-2-yl)phenyl)amino)-1,2,4- |
| | triazine-6-carboxamide; |
| E-56 | benzyl (4-((3-(((R)-1-acryloylpiperidin-3-yl)amino)-6-carbamoyl-1,2,4-triazin-5- |
| | yl)amino)benzyl)((S)-3,3-dimethylbutan-2-yl)carbamate; |
| E-57 | 3-(((R)-1-acryloylpiperidin-3-yl)amino)-5-((4-((((S)-3,3-dimethylbutan-2- |
| | yl)amino)methyl)phenyl)amino)-1,2,4-triazine-6-carboxamide; |
| E-58 | 3-(((1R,3R)-3-acrylamidocyclohexyl)amino)-5-((4-isopropylphenyl)amino)-1,2,4- |
| | triazine-6-carboxamide; |
| E-59 | 3-(((1R,3S)-3-acrylamidocyclohexyl)amino)-5-((4-isopropylphenyl)amino)-1,2,4- |
| | triazine-6-carboxamide; |
| E-60 | (R)-5-((4-(1-acryloyl-1H-pyrazol-4-yl)phenyl)amino)-3-((1-acryloylpiperidin-3- |
| | yl)amino)-1,2,4-triazine-6-carboxamide; |
| E-61 | (R)-5-((3-(2H-1,2,3-triazol-2-yl)phenyl)amino)-3-((1-acryloylpiperidin-3-yl)amino)- |
| | 1,2,4-triazine-6-carboxamide; |
| E-62 | (R)-3-((1-acryloylpiperidin-3-yl)amino)-5-((4-(3-oxomorpholino)phenyl)amino)-1,2,4- |
| | triazine-6-carboxamide; |
| E-63 | (R)-3-((1-acryloylpiperidin-3-yl)amino)-5-((4-(thiazol-2-yl)phenyl)amino)-1,2,4-triazine- |
| | 6-carboxamide; |
| E-64 | (R)-5-((4-(2H-tetrazol-5-yl)phenyl)amino)-3-((1-acryloylpiperidin-3-yl)amino)-1,2,4- |
| | triazine-6-carboxamide; |
| E-65 | (R)-3-((1-acryloylpiperidin-3-yl)amino)-5-((3-(oxazol-2-yl)phenyl)amino)-1,2,4-triazine- |
| | 6-carboxamide; |
| E-66 | (R)-3-((1-acryloylpiperidin-3-yl)amino)-5-((1-ethyl-1H-indazol-5-yl)amino)-1,2,4- |
| | triazine-6-carboxamide; |
| E-67 | (R)-5-((4-(2H-1,2,3-triazol-2-yl)phenyl)amino)-3-((1-acryloylpiperidin-3-yl)amino)- |
| | 1,2,4-triazine-6-carboxamide; |
| E-68 | (R)-3-((1-acryloylpiperidin-3-yl)amino)-5-(quinolin-6-ylamino)-1,2,4-triazine-6- |
| | carboxamide; |
| E-69 | (R)-5-((4-(1H-1,2,4-triazol-1-yl)phenyl)amino)-3-((1-acryloylpiperidin-3-yl)amino)- |
| | 1,2,4-triazine-6-carboxamide; |
| E-70 | (R)-3-((1-acryloylpiperidin-3-yl)amino)-5-((3-(thiazol-2-yl)phenyl)amino)-1,2,4-triazine- |
| | 6-carboxamide; |
| E-71 | (R)-3-((1-acryloylpiperidin-3-yl)amino)-5-((1-methyl-2-oxo-1,2,3,4-tetrahydroquinolin- |
| | 6-yl)amino)-1,2,4-triazine-6-carboxamide; |
| E-72 | tert-butyl (R)-3-((6-carbamoyl-5-((1-methyl-2-oxo-1,2,3,4-tetrahydroquinolin-6- |
| | yl)amino)-1,2,4-triazin-3-yl)amino)piperidine-1-carboxylate; |
| E-73 | (R)-3-((1-acryloylpiperidin-3-yl)amino)-5-((1-methyl-1H-pyrazol-4-yl)amino)-1,2,4- |
| | triazine-6-carboxamide; |
| E-74 | (R)-3-((1-acryloylpiperidin-3-yl)amino)-5-((4-chlorophenyl)amino)-1,2,4-triazine-6- |
| | carboxamide; |
| E-75 | (R)-3-((1-acryloylpiperidin-3-yl)amino)-5-((3-chlorophenyl)amino)-1,2,4-triazine-6- |
| | carboxamide; |
| E-76 | (R)-3-((1-acryloylpiperidin-3-yl)amino)-5-((3-chloro-5-fluorophenyl)amino)-1,2,4- |
| | triazine-6-carboxamide; |
| E-77 | (R)-3-((1-acryloylpiperidin-3-yl)amino)-5-((3-cyanophenyl)amino)-1,2,4-triazine-6- |
| | carboxamide; |
| E-78 | (R)-3-((1-acryloylpiperidin-3-yl)amino)-5-((4-(pentefluoro-16-sulfanyl)phenyl)amino)- |
| | 1,2,4-triazine-6-carboxamide; |
| E-79 | (R)-3-((1-acryloylpiperidin-3-yl)amino)-5-((4-cyanophenyl)amino)-1,2,4-triazine-6- |
| | carboxamide; |
| E-80 | (R)-3-((1-acryloylpiperidin-3-yl)amino)-5-((4-(1-methyl-4,5-dihydro-1H-imidazol-2- |
| | yl)phenyl)amino)-1,2,4-triazine-6-carboxamide; |
| E-81 | ethyl (R)-3-((3-((1-acryloylpiperidin-3-yl)amino)-6-carbamoyl-1,2,4-triazin-5- |
| | yl)amino)benzoate; |
| E-82 | (R)-3-((1-acryloylpiperidin-3-yl)amino)-5-((3-(methylcarbamoyl)phenyl)amino)-1,2,4- |
| | triazine-6-carboxamide; |
| E-83 | (R)-3-((1-acryloylpiperidin-3-yl)amino)-5-((3-(dimethylcarbamoyl)phenyl)amino)-1,2,4- |
| | triazine-6-carboxamide; |
| E-84 | (R)-3-((1-acryloylpiperidin-3-yl)amino)-5-((3-(cyclopropylcarbamoyl)phenyl)amino)- |
| | 1,2,4-triazine-6-carboxamide; |
| E-85 | (R)-3-((1-acryloylpiperidin-3-yl)amino)-5-((3-(morpholine-4-carbonyl)phenyl)amino)- |
| | 1,2,4-triazine-6-carboxamide; |
| E-86 | (R)-3-((1-acryloylpiperidin-3-yl)amino)-5-((3-(benzylcarbamoyl)phenyl)amino)-1,2,4- |
| | triazine-6-carboxamide; |
| E-87 | (R)-3-((1-acryloylpiperidin-3-yl)amino)-5-((3-(piperidine-1-carbonyl)phenyl)amino)- |
| | 1,2,4-triazine-6-carboxamide; |
| E-88 | (R)-3-(2-(acrylamidomethyl)piperidin-1-yl)-5-((4-isopropylphenyl)amino)-1,2,4-triazine- |
| | 6-carboxamide; |
| E-89 | (S)-3-(2-(acrylamidomethyl)piperidin-1-yl)-5-((4-isopropylphenyl)amino)-1,2,4-triazine- |
| | 6-carboxamide; |
| E-90 | (R)-3-(2-(acrylamidomethyl)pyrrolidin-1-yl)-5-((4-isopropylphenyl)amino)-1,2,4- |
| | triazine-6-carboxamide; |
| E-91 | (S)-3-(2-(acrylamidomethyl)pyrrolidin-1-yl)-5-((4-isopropylphenyl)amino)-1,2,4- |
| | triazine-6-carboxamide; |
| E-92 | (R)-5-((1-acryloylpiperidin-3-yl)(methyl)amino)-3-((4-(1-cyclopentylpiperidin-4- |
| | yl)phenyl)amino)pyrazine-2-carboxamide; |
| E-93 | 5-((2R,3R)-3-acrylamido-2-methylpiperidin-1-yl)-3-((4-(1-cyclopropylpiperidin-4- |
| | yl)phenyl)amino)pyrazine-2-carboxamide; |
| E-94 | (S)-5-((1-acryloylpiperidin-3-yl)amino)-3-((4-(pyrimidin-2-yl)phenyl)amino)pyrazine-2- |
| | carboxamide; |
| E-95 | 3-((2R,3R)-3-acrylamido-2-methylpiperidin-1-yl)-5-((4-chlorophenyl)amino)-1,2,4- |
| | triazine-6-carboxamide; |
| E-96 | 3-(((2R,3R)-1-acryloyl-2-methylpiperidin-3-yl)amino)-5-((4-chlorophenyl)amino)-1,2,4- |
| | triazine-6-carboxamide; |
| E-97 | 5-((2R,3R)-3-acrylamido-2-methylpiperidin-1-yl)-3-((3-methylisothiazol-5- |
| | yl)amino)pyrazine-2-carboxamide; |
| E-98 | 5-((2R,3R)-3-acrylamido-2-methylpiperidin-1-yl)-3-((1-methyl-1H-pyrazol-4- |
| | yl)amino)pyrazine-2-carboxamide; |
| E-99 | 5-(((2R,3R)-1-acryloyl-2-methylpiperidin-3-yl)amino)-3-((4-(1-cyclopentylpiperidin-4- |
| | yl)phenyl)amino)pyrazine-2-carboxamide; |
| E-100 | 5-(((2R,3R)-1-acryloyl-2-methylpiperidin-3-yl)amino)-3-((4-(1-cyclopentyl-4- |
| | methylpiperidin-4-yl)phenyl)amino)pyrazine-2-carboxamide; |
| E-101 | (S)-5-((1-acryloylpiperidin-3-yl)amino)-3-((4-isopropylphenyl)amino)pyrazine-2- |
| | carboxamide; |
| E-102 | (R)-5-((1-acryloylpiperidin-3-yl)(methyl)amino)-3-((4-isopropylphenyl)amino)pyrazine- |
| | 2-carboxamide; |
| E-103 | (R)-5-((1-acryloylpiperidin-3-yl)(methyl)amino)-3-((4-(1-cyclopentyl-4-methylpiperidin- |
| | 4-yl)phenyl)amino)pyrazine-2-carboxamide; |
| E-104 | (R)-5-((1-acryloylpyrrolidin-3-yl)(methyl)amino)-3-((4-(1-cyclopropyl-4- |
| | methylpiperidin-4-yl)phenyl)amino)pyrazine-2-carboxamide; |
| E-105 | (R)-3-((4-(1-cyclopropyl-4-methylpiperidin-4-yl)phenyl)amino)-5-(methyl(1-(2,2,2- |
| | trifluoroacetyl)pyrrolidin-3-yl)amino)pyrazine-2-carboxamide; |
| E-106 | (R)-5-((1-acryloylpyrrolidin-3-yl)(methyl)amino)-3-((4-(1-cyclopentyl-4- |
| | methylpiperidin-4-yl)phenyl)amino)pyrazine-2-carboxamide; |
| E-107 | (R)-3-((4-(1-cyclopentyl-4-methylpiperidin-4-yl)phenyl)amino)-5-(methyl(1-(2,2,2- |
| | trifluoroacetyl)pyrrolidin-3-yl)amino)pyrazine-2-carboxamide; |
| E-108 | (R)-5-((1-acryloylpiperidin-3-yl)amino)-3-((4-(1-cyclopentylpiperidin-4- |
| | yl)phenyl)amino)pyrazine-2-carboxamide; |
| E-109 | (R)-5-((1-acryloylpyrrolidin-3-yl)(methyl)amino)-3-((4-(1-cyclopentylpiperidin-4- |
| | yl)phenyl)amino)pyrazine-2-carboxamide; |
| E-110 | (R)-5-((1-acryloylpyrrolidin-3-yl)(methyl)amino)-3-((1-(2-methoxyethyl)-1H-pyrazol-4- |
| | yl)amino)pyrazine-2-carboxamide; |
| E-111 | (R)-5-((1-acryloylpyrrolidin-3-yl)(methyl)amino)-3-((4-(4-methylpiperazin-1- |
| | yl)phenyl)amino)pyrazine-2-carboxamide; |
| E-112 | (R)-5-((1-acryloylpyrrolidin-3-yl)(methyl)amino)-3-((3-(1-methyl-1H-imidazol-2- |
| | yl)phenyl)amino)pyrazine-2-carboxamide; |
| E-113 | (R)-3-((3-(2H-1,2,3-triazol-2-yl)phenyl)amino)-5-((1-acryloylpyrrolidin-3- |
| | yl)(methyl)amino)pyrazine-2-carboxamide; |
| E-114 | (R)-5-((1-acryloylpyrrolidin-3-yl)(methyl)amino)-3-((3-methylisothiazol-5- |
| | yl)amino)pyrazine-2-carboxamide; |
| E-115 | (R)-5-((1-acryloylpyrrolidin-3-yl)(methyl)amino)-3-((4-(1-methylpiperidin-4- |
| | yl)phenyl)amino)pyrazine-2-carboxamide; |
| E-116 | (R)-3-((4-(2H-1,2,3-triazol-2-yl)phenyl)amino)-5-((1-acryloylpyrrolidin-3- |
| | yl)(methyl)amino)pyrazine-2-carboxamide; |
| E-117 | (R)-5-((1-acryloylpyrrolidin-3-yl)(methyl)amino)-3-((4-(pyrimidin-2- |
| | yl)phenyl)amino)pyrazine-2-carboxamide; |
| E-118 | (R)-5-((1-acryloylpyrrolidin-3-yl)(methyl)amino)-3-((4-(oxazol-2- |
| | yl)phenyl)amino)pyrazine-2-carboxamide; |
| E-119 | (R)-5-((1-acryloylpyrrolidin-3-yl)(methyl)amino)-3-((1-methyl-1H-pyrazol-4- |
| | yl)amino)pyrazine-2-carboxamide; |
| E-120 | (R)-5-((1-acryloylpyrrolidin-3-yl)(methyl)amino)-3-((4-(1,1,1,3,3,3-hexafluoro-2- |
| | hydroxypropan-2-yl)phenyl)amino)pyrazine-2-carboxamide; |
| E-121 | (R)-5-((1-acryloylpyrrolidin-3-yl)(methyl)amino)-3-((4-(1-cyclopropylpiperidin-4- |
| | yl)phenyl)amino)pyrazine-2-carboxamide; |
| E-122 | (R)-5-((1-(3-chloropropanoyl)pyrrolidin-3-yl)(methyl)amino)-3-((4-(1- |
| | cyclopropylpiperidin-4-yl)phenyl)amino)pyrazine-2-carboxamide; |
| E-123 | (R)-5-((1-(3-chloropropanoyl)pyrrolidin-3-yl)(methyl)amino)-3- |
| | (cyclopropylamino)pyrazine-2-carboxamide; |
| E-124 | (R)-3-((1-acryloylpyrrolidin-3-yl)amino)-5-((3-methylisothiazol-5-yl)amino)-1,2,4- |
| | triazine-6-carboxamide; |
| E-125 | (R)-5-((1-acryloylpiperidin-3-yl)(methyl)amino)-3-((4-(1-cyclopropylpiperidin-4- |
| | yl)phenyl)amino)pyrazine-2-carboxamide; |
| E-126 | (R)-5-((1-acryloylpiperidin-3-yl)(methyl)amino)-3-((3-methylisothiazol-5- |
| | yl)amino)pyrazine-2-carboxamide; |
| E-127 | 3-(((2R,3R)-1-acryloyl-2-methylpiperidin-3-yl)amino)-5-((4-(1-cyclopropyl-4- |
| | methylpiperidin-4-yl)phenyl)amino)-1,2,4-triazine-6-carboxamide |
| E-128 | 3-((3R,3′R)-3-((3-chloro-5-(trifluoromethyl)phenyl)amino)-2-oxo-[1,3′-bipiperidin]-1′- |
| | yl)-5-((4-isopropylphenyl)amino)-1,2,4-triazine-6-carboxamide |
| E-129 | 3-((3R,3′R,4S)-3-((3-chloro-5-(trifluoromethyl)phenyl)amino)-4-hydroxy-2-oxo-[1,3′- |
| | bipiperidin]-1′-yl)-5-((4-isopropylphenyl)amino)-1,2,4-triazine-6-carboxamide |
| E-130 | 5-((4-isopropylphenyl)amino)-3-((3R,3′R)-2-oxo-3-((3-(trifluoromethyl)phenyl)amino)- |
| | [1,3′-bipiperidin]-1′-yl)-1,2,4-triazine-6-carboxamide |
| E-131 | 5-((4-isopropylphenyl)amino)-3-((3R,3′R)-2-oxo-3-((4-(trifluoromethyl)phenyl)amino)- |
| | [1,3′-bipiperidin]-1′-yl)-1,2,4-triazine-6-carboxamide |
| E-132 | rac-3-((3R,4S)-3-acrylamido-4-fluoropiperidin-1-yl)-5-((4-isopropylphenyl)amino)- |
| | 1,2,4-triazine-6-carboxamide |
| E-133 | 3-(3-acrylamido-3-methylpiperidin-1-yl)-5-((4-isopropylphenyl)amino)-1,2,4-triazine-6- |
| | carboxamide |
| E-134 | (R)-5-((4-(2H-1,2,3-triazol-2-yl)phenyl)amino)-3-((1-acryloylpiperidin-3- |
| | yl)(methyl)amino)-1,2,4-triazine-6-carboxamide |
| E-135 | (R)-3-((1-acryloylpyrrolidin-3-yl)(methyl)amino)-5-((4-(1-cyclopropylpiperidin-4- |
| | yl)phenyl)amino)-1,2,4-triazine-6-carboxamide |
| E-136 | 5-((2R,3R)-3-(4-cyclopropylbenzamido)-2-methylpiperidin-1-yl)-3-((4-((1- |
| | methylpyrrolidin-3-yl)oxy)phenyl)amino)pyrazine-2-carboxamide |
| E-137 | 3-((4-((S)-4-cyclopentyl-2-methylpiperazin-1-yl)phenyl)amino)-5-((2R,3R)-3-(4- |
| | cyclopropylbenzamido)-2-methylpiperidin-1-yl)pyrazine-2-carboxamide |
| E-138 | N1-((2R,3R)-1-(5-carbamoyl-6-((1-methyl-1H-pyrazol-4-yl)amino)pyrazin-2-yl)-2- |
| | methylpiperidin-3-yl)-N4,N4-dimethylterephthalamide |
| E-139 | N-((2R,3R)-1-(5-carbamoyl-6-((1-methyl-1H-pyrazol-4-yl)amino)pyrazin-2-yl)-2- |
| | methylpiperidin-3-yl)-2-cyclopropylbenzo[d]oxazole-5-carboxamide |
| E-140 | 5-((3R,4R)-3-(4-cyclopropylbenzamido)-4-methylpiperidin-1-yl)-3-((1-methyl-1H- |
| | pyrazol-4-yl)amino)pyrazine-2-carboxamide |
| E-141 | 5-(1-(4-cyclopropylbenzoyl)octahydro-6H-pyrrolo[2,3-c]pyridin-6-yl)-3-((1-methyl-1H- |
| | pyrazol-4-yl)amino)pyrazine-2-carboxamide |
| E-142 | 5-((2R,3R)-3-(4-cyclopropylbenzamido)-2-methylpiperidin-1-yl)-3-((4-((S)-2-methyl-4- |
| | (oxetan-3-yl)piperazin-1-yl)phenyl)amino)pyrazine-2-carboxamide |
| E-143 | 3-((1-methyl-1H-pyrazol-4-yl)amino)-5-((2R,3R)-2-methyl-3-(4-(2-(pyrrolidin-1- |
| | yl)propan-2-yl)benzamido)piperidin-1-yl)pyrazine-2-carboxamide |
| E-144 | 5-((2R,3R)-3-(4-cyclopropylbenzamido)-2-methylpiperidin-1-yl)-3-((2-(tetrahydro-2H- |
| | pyran-4-yl)pyrimidin-5-yl)amino)pyrazine-2-carboxamide |
| E-145 | 5-((2R,3R)-3-(3-(2-hydroxypropan-2-yl)benzamido)-2-methylpiperidin-1-yl)-3-((1- |
| | methyl-1H-pyrazol-4-yl)amino)pyrazine-2-carboxamide |
| E-146 | 5-((2R,3R)-3-(3-(tert-butyl)-1-methyl-1H-pyrazole-5-carboxamido)-2-methylpiperidin-1- |
| | yl)-3-((1-methyl-1H-pyrazol-4-yl)amino)pyrazine-2-carboxamide |
| E-147 | 3-((4-(1-cyclopropyl-4-methylpiperidin-4-yl)phenyl)amino)-5-((2R,3R)-3-(4-(2- |
| | hydroxypropan-2-yl)benzamido)-2-methylpiperidin-1-yl)pyrazine-2-carboxamide |
| E-148 | 5-((2R,3R)-3-(4-cyclopropylbenzamido)-2-methylpiperidin-1-yl)-3-((4-((S)-2,4- |
| | dimethylpiperazin-1-yl)phenyl)amino)pyrazine-2-carboxamide |
| E-149 | 5-(tert-butyl)-N-((2R,3R)-1-(5-carbamoyl-6-((1-methyl-1H-pyrazol-4-yl)amino)pyrazin- |
| | 2-yl)-2-methylpiperidin-3-yl)-1,2,4-oxadiazole-3-carboxamide |
| E-150 | 5-(3-(4-cyclopropylbenzamido)-3-methylpiperidin-1-yl)-3-((1-methyl-1H-pyrazol-4- |
| | yl)amino)pyrazine-2-carboxamide |
| E-151 | 5-((2R,3R)-3-(4-(3-hydroxypentan-3-yl)benzamido)-2-methylpiperidin-1-yl)-3-((1- |
| | methyl-1H-pyrazol-4-yl)amino)pyrazine-2-carboxamide |
| E-152 | 5-((2R,3R)-3-(4-(2-hydroxypropan-2-yl)-3-methylbenzamido)-2-methylpiperidin-1-yl)- |
| | 3-((1-methyl-1H-pyrazol-4-yl)amino)pyrazine-2-carboxamide |
| E-153 | 5-((2R,3R)-3-(3,3-dimethyl-1,3-dihydroisobenzofuran-5-carboxamido)-2- |
| | methylpiperidin-1-yl)-3-((1-methyl-1H-pyrazol-4-yl)amino)pyrazine-2-carboxamide |
| E-154 | 3-((1-methyl-1H-pyrazol-4-yl)amino)-5-((2R,3R)-2-methyl-3-(4-(oxazol-2- |
| | yl)benzamido)piperidin-1-yl)pyrazine-2-carboxamide |
| E-155 | 3-((1-methyl-1H-pyrazol-4-yl)amino)-5-((2R,3R)-2-methyl-3-(4-(2-methylthiazol-4- |
| | yl)benzamido)piperidin-1-yl)pyrazine-2-carboxamide |
| E-156 | 3-((2R,3R)-3-(3-fluoro-4-(2-hydroxypropan-2-yl)benzamido)-2-methylpiperidin-1-yl)-5- |
| | ((1-methyl-1H-pyrazol-4-yl)amino)-1,2,4-triazine-6-carboxamide |
| E-157 | 5-((3S,4R)-3-(4-cyclopropylbenzamido)-4-fluoropiperidin-1-yl)-3-((1-methyl-1H- |
| | pyrazol-4-yl)amino)pyrazine-2-carboxamide |
| E-158 | 5-((2R,3R)-3-(5-(2-hydroxypropan-2-yl)picolinamido)-2-methylpiperidin-1-yl)-3-((1- |
| | methyl-1H-pyrazol-4-yl)amino)pyrazine-2-carboxamide |

In some embodiments, the compound is selected from:

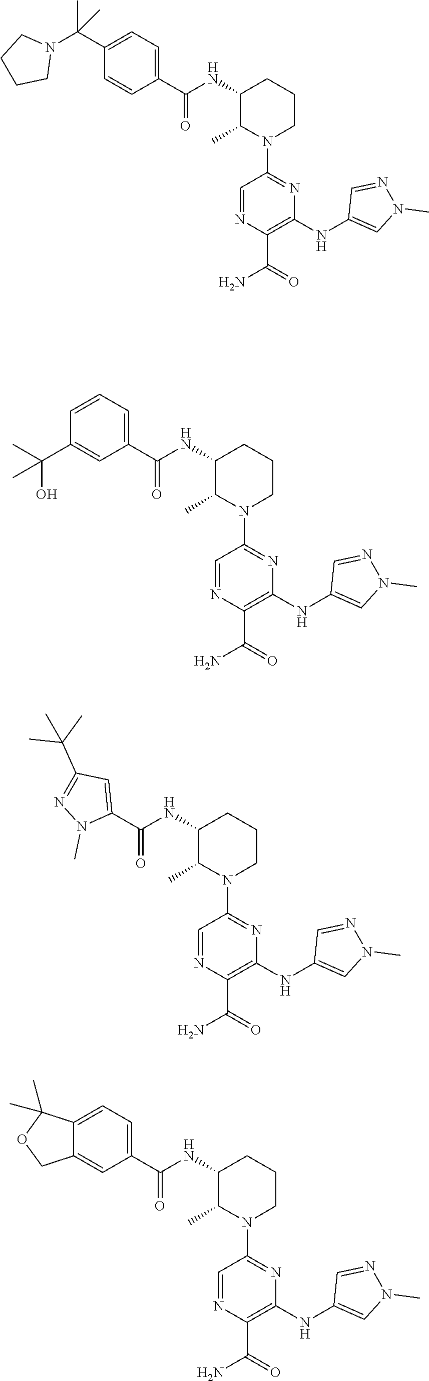

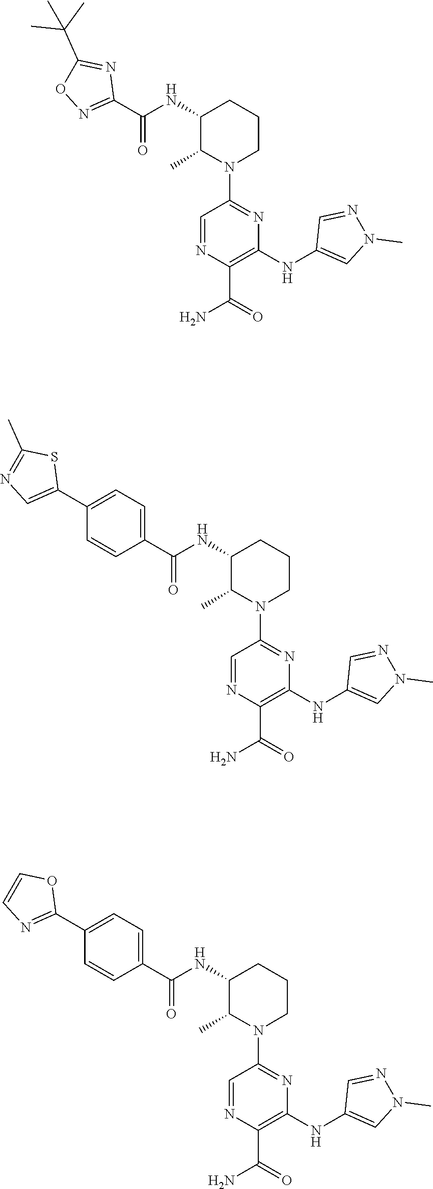 [SMILES: C[C@@H]1[C@H](NC(=O)c2noc(C(C)(C)C)n2)CCCN1c1cnc(C(N)=O)c(Nc2cnn(C)c2)n1 ; Cc1ncc(-c2ccc(C(=O)N[C@@H]3CCCN(c4cnc(C(N)=O)c(Nc5cnn(C)c5)n4)[C@@H]3C)cc2)s1 ; C[C@@H]1[C@H](NC(=O)c2ccc(-c3ncco3)cc2)CCCN1c1cnc(C(N)=O)c(Nc2cnn(C)c2)n1]

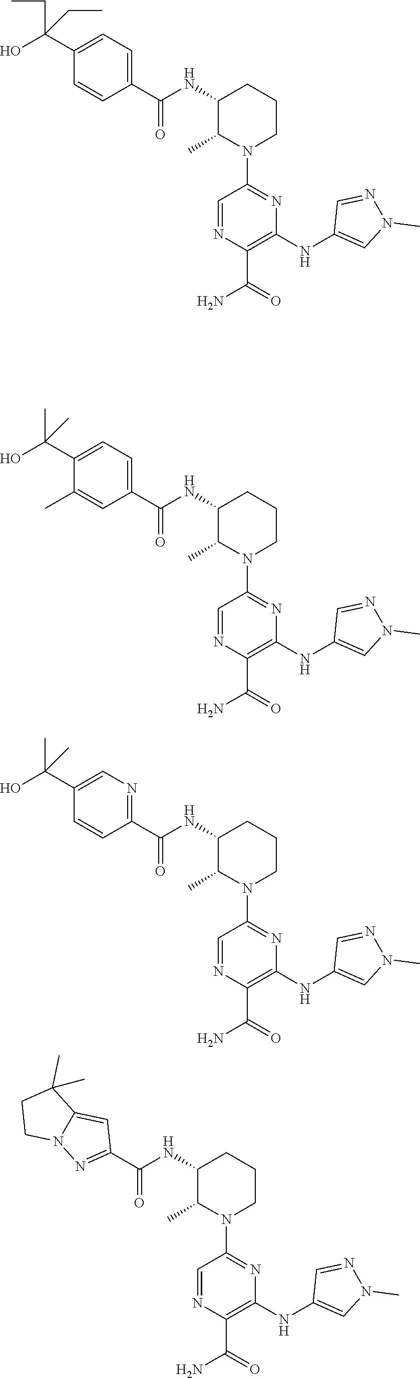 [SMILES: CCC(O)(CC)c1ccc(C(=O)N[C@@H]2CCCN(c3cnc(C(N)=O)c(Nc4cnn(C)c4)n3)[C@@H]2C)cc1 ; Cc1cc(C(=O)N[C@@H]2CCCN(c3cnc(C(N)=O)c(Nc4cnn(C)c4)n3)[C@@H]2C)ccc1C(C)(C)O ; C[C@@H]1[C@H](NC(=O)c2ccc(C(C)(C)O)cn2)CCCN1c1cnc(C(N)=O)c(Nc2cnn(C)c2)n1 ; C[C@@H]1[C@H](NC(=O)c2cc3n(n2)CCC3(C)C)CCCN1c1cnc(C(N)=O)c(Nc2cnn(C)c2)n1]

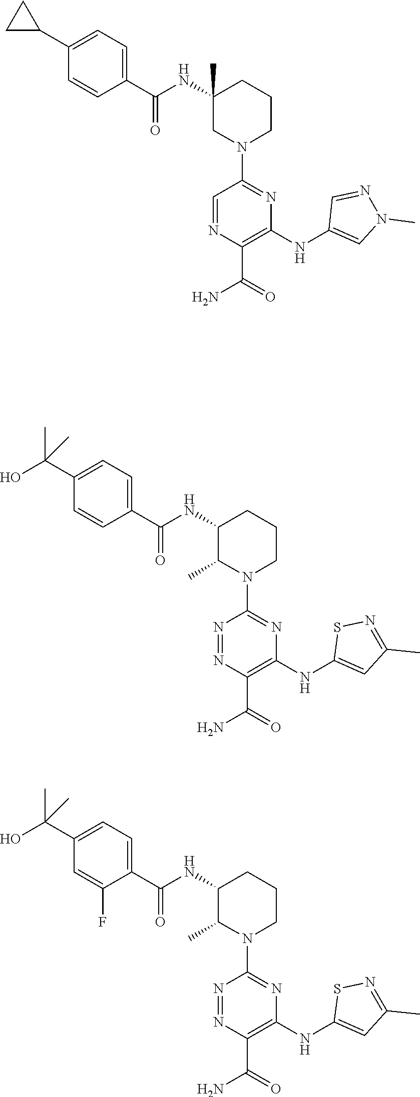 [SMILES: Cn1cc(Nc2nc(N3CCC[C@@](C)(NC(=O)c4ccc(C5CC5)cc4)C3)cnc2C(N)=O)cn1 ; Cc1cc(Nc2nc(N3CCC[C@@H](NC(=O)c4ccc(C(C)(C)O)cc4)[C@H]3C)nnc2C(N)=O)sn1 ; Cc1cc(Nc2nc(N3CCC[C@@H](NC(=O)c4ccc(C(C)(C)O)cc4F)[C@H]3C)nnc2C(N)=O)sn1]

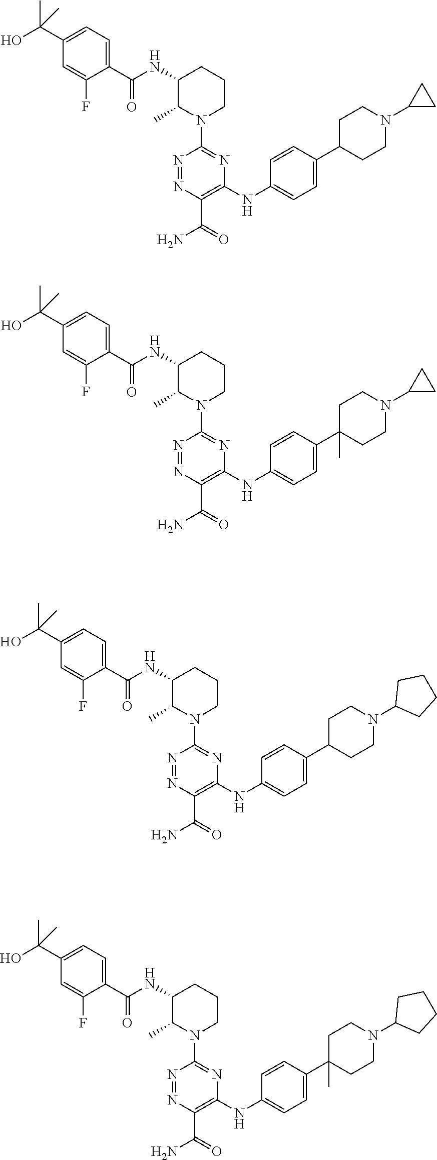 [SMILES: C[C@@H]1[C@H](NC(=O)c2ccc(C(C)(C)O)cc2F)CCCN1c1nnc(C(N)=O)c(Nc2ccc(C3CCN(C4CC4)CC3)cc2)n1 ; C[C@@H]1[C@H](NC(=O)c2ccc(C(C)(C)O)cc2F)CCCN1c1nnc(C(N)=O)c(Nc2ccc(C3(C)CCN(C4CC4)CC3)cc2)n1 ; C[C@@H]1[C@H](NC(=O)c2ccc(C(C)(C)O)cc2F)CCCN1c1nnc(C(N)=O)c(Nc2ccc(C3CCN(C4CCCC4)CC3)cc2)n1 ; C[C@@H]1[C@H](NC(=O)c2ccc(C(C)(C)O)cc2F)CCCN1c1nnc(C(N)=O)c(Nc2ccc(C3(C)CCN(C4CCCC4)CC3)cc2)n1]

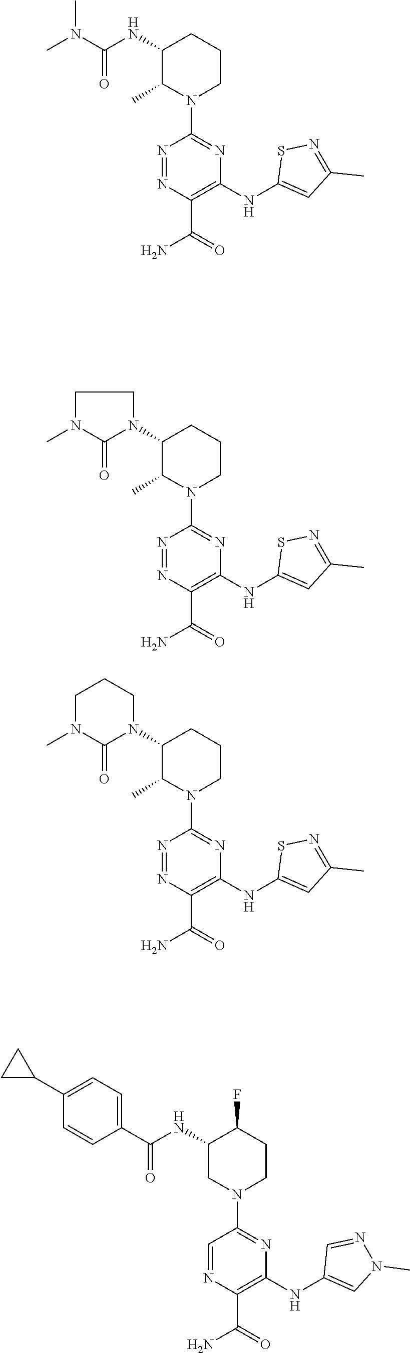

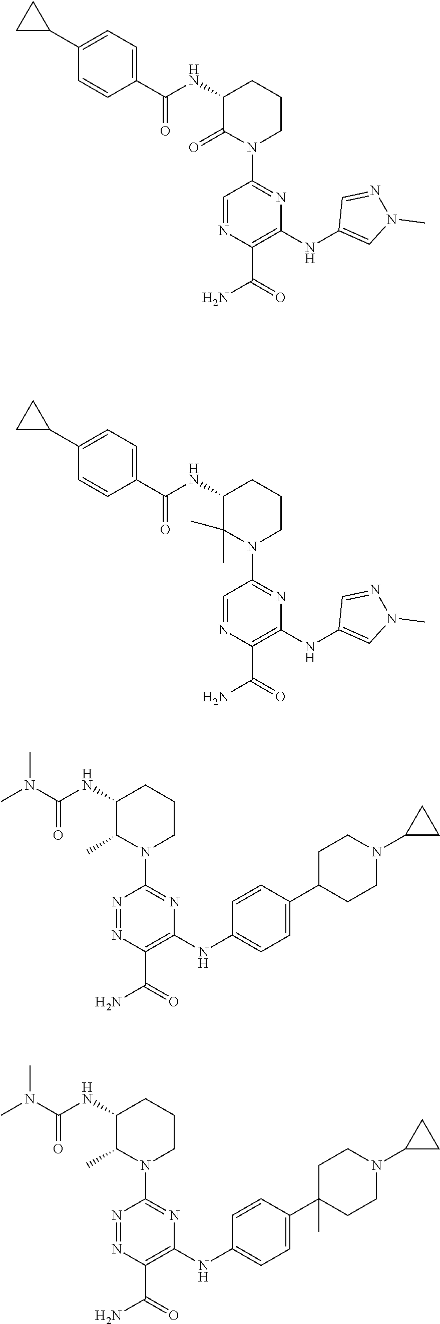

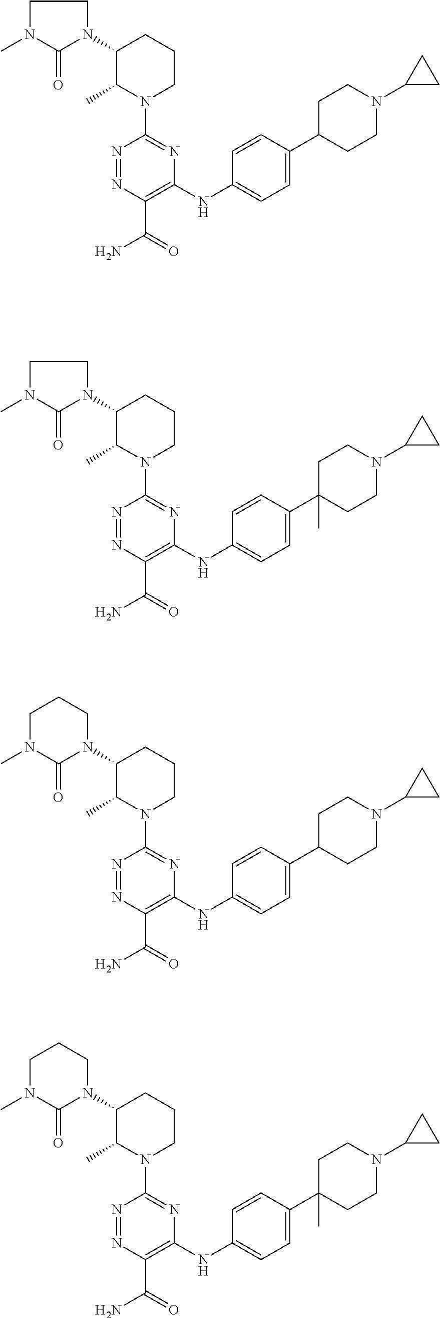 [SMILES: C[C@@H]1[C@H](N2CCN(C)C2=O)CCCN1c1nnc(C(N)=O)c(Nc2ccc(C3CCN(C4CC4)CC3)cc2)n1 ; C[C@@H]1[C@H](N2CCN(C)C2=O)CCCN1c1nnc(C(N)=O)c(Nc2ccc(C3(C)CCN(C4CC4)CC3)cc2)n1 ; C[C@@H]1[C@H](N2CCCN(C)C2=O)CCCN1c1nnc(C(N)=O)c(Nc2ccc(C3CCN(C4CC4)CC3)cc2)n1 ; C[C@@H]1[C@H](N2CCCN(C)C2=O)CCCN1c1nnc(C(N)=O)c(Nc2ccc(C3(C)CCN(C4CC4)CC3)cc2)n1]

In some embodiments, the compound is a deuterated analog of any one of the compounds described herein.

In some embodiments, the compound is not a compound described in U.S. patent application Ser. No. 14/559,889 or International Patent Application PCT/US2014/68434, both of which were filed on Dec. 3, 2014.

At least some of the chemical names of compounds of the invention as given and set forth in this application, may have been generated on an automated basis by use of a commercially available chemical naming software program, and have not been independently verified. Representative programs performing this function include the ChemDraw naming tool sold by Cambridge Software, Inc. and the Instant JChem Software tool sold by ChemAxon, Inc. In the instance where the indicated chemical name and the depicted structure differ, the depicted structure will control.

### Preparation of Compounds

Compounds described herein may be synthesized using standard synthetic reactions known to those of skill in the art or using methods known in the art. The reactions can be employed in a linear sequence to provide the compounds or they may be used to synthesize fragments which are subsequently joined by the methods known in the art.

Described herein are compounds that inhibit the activity of tyrosine kinase(s), such as Btk, and processes for their preparation. Also described herein are pharmaceutically acceptable salts, pharmaceutically acceptable solvates, pharmaceutically active metabolites and pharmaceutically acceptable prodrugs of such compounds. Pharmaceutical compositions that include at least one such compound or a pharmaceutically acceptable salt, pharmaceutically acceptable solvate, pharmaceutically active metabolite or pharmaceutically acceptable prodrug of such compound, are provided.

The starting material used for the synthesis of the compounds described herein may be synthesized or can be obtained from commercial sources, such as, but not limited to, Aldrich Chemical Co. (Milwaukee, Wis.), Bachem (Torrance, Calif.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). All of the foregoing publications are incorporated herein by reference in their entireties. Other methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography and the like. Such materials may be characterized using conventional means, including physical constants and spectral data.

Compounds described herein may be prepared as a single isomer or a mixture of isomers.

In some aspects, the compounds of Formula (A-I) are prepared according to Scheme A wherein A, L, X¹, X², Y, Z, R¹, R⁴, R⁵, R¹⁰, m, n and p are as defined herein, W is C(O)NH₂ or a group that can be converted to C(O)NH₂, such as CN or an ester, and LG¹ and LG² are independently a leaving group, such as halo, tosylate or triflate, or a group that can be converted to a leaving group, such as SCH₃. In Scheme A, Compound A-1 reacts with Compound A-2 under conditions, such as in the presence of a base (e.g., TEA, DIEA, pyridine, etc.) and a solvent (such as DMF, DCM, etc.), to form Compound A-3. Compound A-3 reacts with Compound A-4 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (A-I) when W is C(O)NH₂. In cases where LG² is a group that can be converted to a leaving group, it is converted to a leaving group before reacting with Compound A-4, for example, SCH₃ is first converted to SO₂CH₃ by oxidation. The conversion of LG² and reaction with Compound A-4 can be done without isolation of the intermediate. In certain embodiments, LG¹ and LG² are the same. In certain embodiments, LG¹ is a more reactive leaving group as compared to LG². For example, LG¹ is bromo and LG² is chloro. Reactivity of leaving groups is generally known in the art. Alternatively, Compound A-1 reacts with Compound A-4 under conditions, such as in the presence of a base (e.g., TEA, DIEA, pyridine, etc.) and a solvent (such as DMF, DCM, etc.), to form Compound A-5. Compound A-5 reacts with Compound A-2 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (A-I) when W is C(O)NH₂; or through a Buchwald coupling-type reaction with a Pd catalyst (e.g. Pd(OAc)₂, Pd(dba)₂, Pd₂(dba)₃, etc.), a ligand (e.g. BINAP, XantPhos, Q-Phos, etc.), a base (Cs₂CO₃, K₂CO₃, tBuOK, etc) in solvent (e.g. dioxane, toluene, etc) under elevated temperature (e.g., 100-120° C.) in nitrogen or argon atmosphere, to form a compound of Formula (A-I) when W is CN, C(O)Me or C(O)Et. Then the CN group is converted to C(O)NH₂ to form the compound of Formula (A-I) through nitrile hydrolysis (e.g., via H₂O₂/DMSO with base, such as NaOH, Cs₂CO₃ or K₂CO₃; or via H₂SO₄/TFA under elevated temperature (e.g., 60-80° C.)) or through saponification and then amidation from the ester.

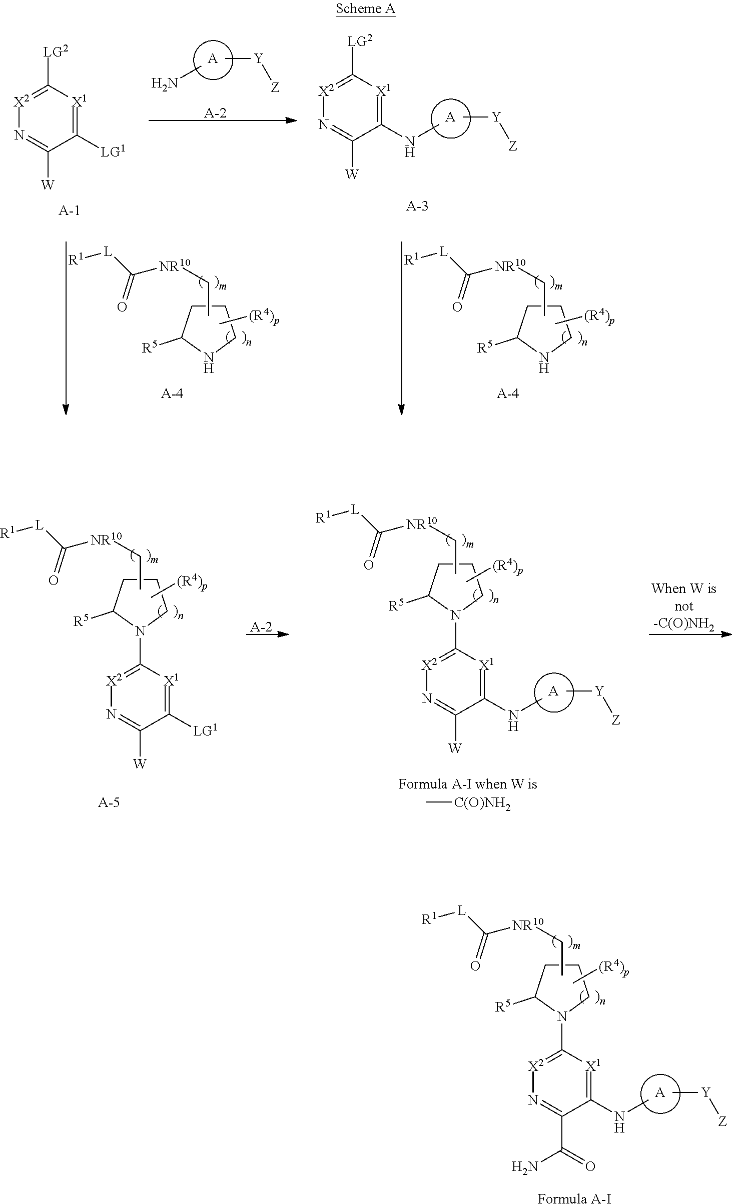

In some aspects, the compounds of Formula (A-I) are prepared according to Scheme B wherein A, L, X¹, X², Y, Z, R¹, R⁴, R⁵, R¹⁰, n and p are as defined herein, W² is OH, halo, or C(O)W² is an active ester or anhydride, and PG¹ is an amino acid protecting group. Many active esters, anhydride groups are known in the art. Many amino acid protecting groups and respective methods of deprotection are also known in the art. Compound B-1 can be prepared according to Scheme A. In Scheme B, Compound B-1 is first deprotected to give the free amino group which reacts with R¹-L-C(O)W² or R¹—NCO under conditions generally known in the art to give a compound of Formula (A-I).

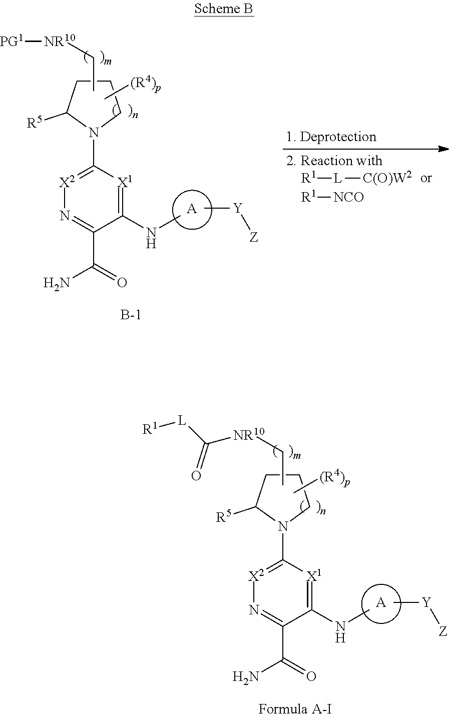

In some aspects, the compounds of Formula (B-I) are prepared according to Scheme A′ wherein A, X¹, X², Y, Z, R¹, R⁴, R¹, R¹⁰, n and p are as defined herein, W is C(O)NH₂ or a group that can be converted to C(O)NH₂, such as CN or an ester, and LG¹ and LG² are independently a leaving group, such as halo, tosylate or triflate, or a group that can be converted to a leaving group, such as SCH₃. In Scheme A, Compound A-I reacts with Compound A-2 under conditions, such as in the presence of a base (e.g., TEA, DIEA, pyridine, etc.) and a solvent (such as DMF, DCM, etc.), to form Compound A-3. Compound A-3 reacts with Compound A-4 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (B-I) when W is C(O)NH₂. In cases where LG² is a group that can be converted to a leaving group, it is converted to a leaving group before reacting with Compound A-4, for example, SCH₃ is first converted to SO₂CH₃ by oxidation. The conversion of LG² and reaction with Compound A-4 can be done without isolation of the intermediate. In certain embodiments, LG¹ and LG² are the same. In certain embodiments, LG¹ is a more reactive leaving group as compared to LG². For example, LG¹ is bromo and LG² is chloro. Reactivity of leaving groups is generally known in the art. Alternatively, Compound A-1 reacts with Compound A-4 under conditions, such as in the presence of a base (e.g., TEA, DIEA, pyridine, etc.) and a solvent (such as DMF, DCM, etc.), to form Compound A-5. Compound A-5 reacts with Compound A-2 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (B-I) when W is C(O)NH₂; or through a Buchwald coupling-type reaction with a Pd catalyst (e.g., Pd(OAc)₂, Pd(dba)₂, Pd₂(dba)₃, etc.), a ligand (e.g., BINAP, XantPhos, Q-Phos, etc.), a base (Cs₂CO₃, K₂CO₃, tBuOK, etc.) in solvent (e.g., dioxane, toluene, etc) under elevated temperature (100-120° C.) in nitrogen or argon atmosphere, to form a compound of Formula (B-I) when W is CN, C(O)Me or C(O)Et. Then W is converted to C(O)NH₂ to form the compound of Formula (B-I) through nitrile hydrolysis (e.g., via H₂O₂/DMSO with base, such as NaOH, Cs₂CO₃ or K₂CO₃; or via H₂SO₄/TFA under elevated temperature (e.g., 60-80° C.)) or through saponification and then amidation from the ester.

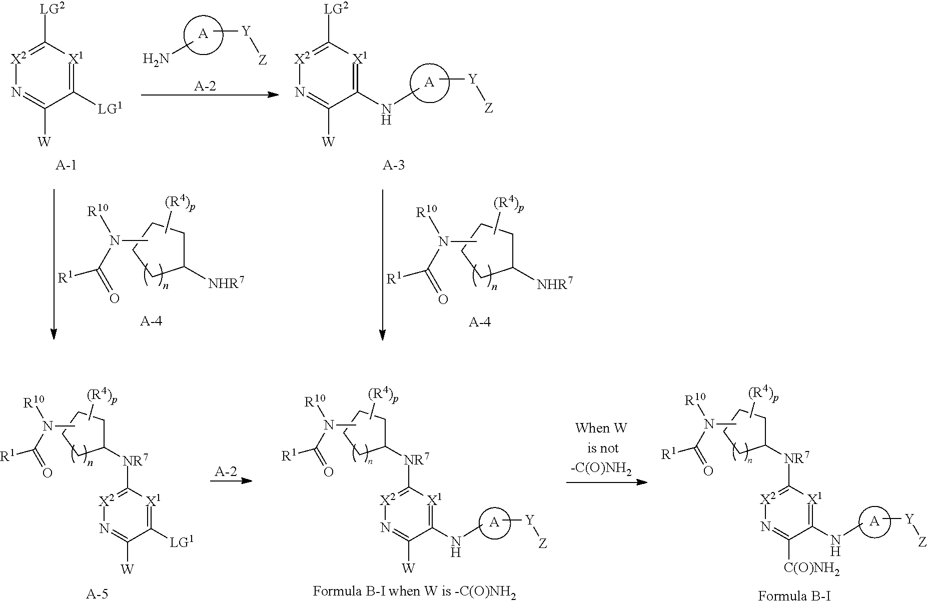

In some aspects, the compounds of Formula (B-I) are prepared according to Scheme B′ wherein A, X¹, X², Y, Z, R¹, R⁴, R⁵, R⁷, R¹⁰, n and p are as defined herein, W² is OH, halo, or C(O)W² is an active ester or anhydride, and PG¹ is an amino acid protecting group. Many active esters, anhydride groups are known in the art. Many amino acid protecting groups and respective methods of deprotection are also known in the art. Compound B-1 can be prepared according to Scheme A. In Scheme B, Compound B-1 is first deprotected to give the free amino group which reacts with R¹C(O)W² or R⁵NCO under conditions generally known in the art to give a compound of Formula (B-I).

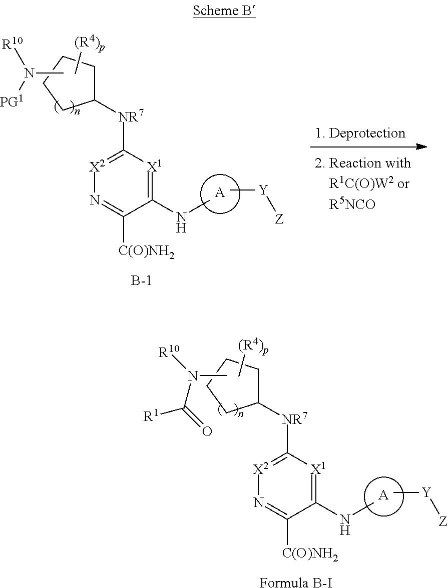

In some aspects, the compounds of Formula (C-I) are prepared according to Scheme A″ wherein A, X, Y, Z, R¹, R⁴, R⁵, m, n and p are as defined herein, W is C(O)NH₂ or a group that can be converted to C(O)NH₂, such as CN or an ester, and LG¹ and LG² are independently a leaving group, such as halo, tosylate or triflate, or a group that can be converted to a leaving group, such as SCH₃. In Scheme A, Compound A-1 reacts with Compound A-2 under conditions, such as in the presence of a base (e.g., TEA, DIEA, pyridine, etc.) and a solvent (such as DMF, DCM, etc.), to form Compound A-3. Compound A-3 reacts with Compound A-4 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (C-I) when W is C(O)NH₂. In cases where LG² is a group that can be converted to a leaving group, it is converted to a leaving group before reacting with Compound A-4, for example, SCH₃ is first converted to SO₂CH₃ by oxidation. The conversion of LG² and reaction with Compound A-4 can be done without isolation of the intermediate. In certain embodiments, LG¹ and LG² are the same. In certain embodiments, LG¹ is a more reactive leaving group as compared to LG². For example, LG¹ is bromo and LG² is chloro. Reactivity of leaving groups is generally known in the art. Alternatively, Compound A-1 reacts with Compound A-4 under conditions, such as in the presence of a base (e.g., TEA, DIEA, pyridine, etc.) and a solvent (such as DMF, DCM, etc.), to form Compound A-5. Compound A-5 reacts with Compound A-2 under conditions, such as in the presence of a base (e.g., TEA, DIEA, pyridine, etc.) and a solvent (such as DMF, DCM, etc.), and optionally under elevated temperatures (such as at about 50-120° C.) to form a compound of Formula (C-I) when W is C(O)NH₂; or through a Buchwald coupling-type reaction with a Pd catalyst (e.g., Pd(OAc)₂, Pd(dba)₂, Pd₂(dba)₃, etc.), a ligand (e.g., BINAP, XantPhos, Q-Phos, etc.), a base (Cs₂CO₃, K₂CO₃, tBuOK, etc) in solvent (e.g. dioxane, toluene, etc) under elevated temperature (100-120° C.) in nitrogen or argon atmosphere, to form a compound of Formula (C-I) when W is CN, C(O)Me or C(O)Et. Then W is converted to C(O)NH₂ to form the compound of Formula (C-I) through nitrile hydrolysis (e.g., via H₂O₂/DMSO with base, such as NaOH, Cs₂CO₃ or K₂CO₃; or via H₂SO₄/TFA under elevated temperature (e.g., 60-80° C.)) or through saponification and then amidation from the ester.

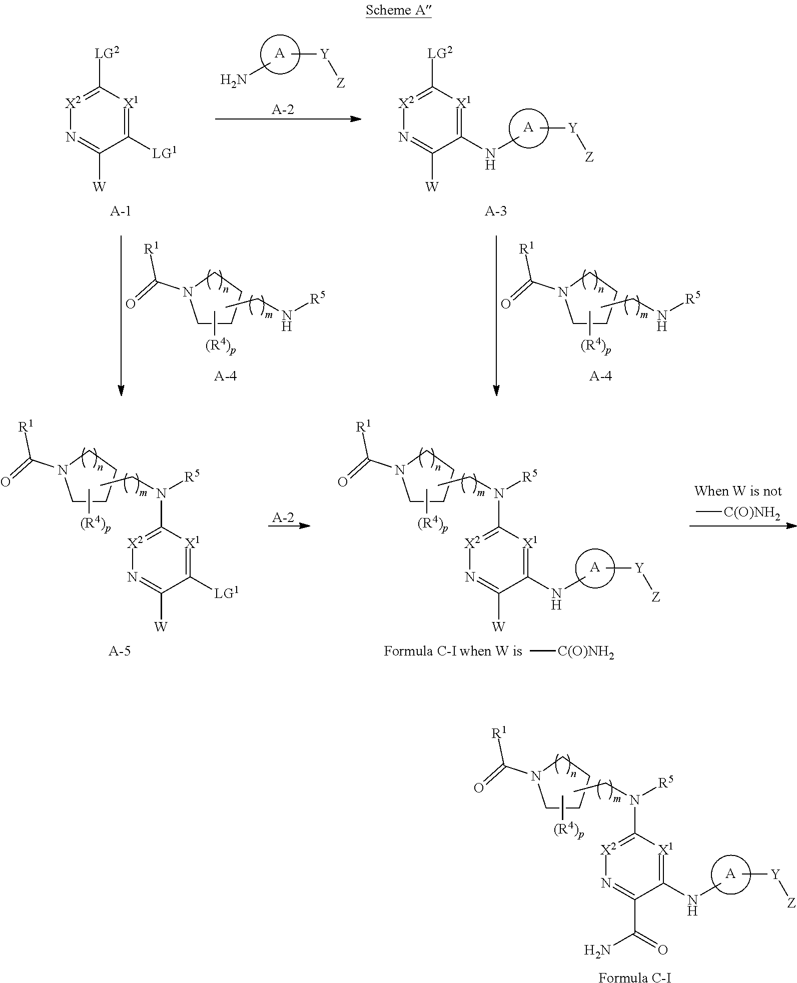

In some aspects, the compounds of Formula (C-I) are prepared according to Scheme B″ wherein A, L, X, Y, Z, R¹, R⁴, R⁵, m, n and p are as defined herein, W² is OH, halo, or C(O)W² is an active ester or anhydride, and PG¹ is an amino acid protecting group. Many active esters, anhydride groups are known in the art. Many amino acid protecting groups and respective methods of deprotection are also known in the art. Compound B-1 can be prepared according to Scheme A. In Scheme B, Compound B-1 is first deprotected to give the free amino group which reacts with R¹C(O)W² under conditions generally known in the art to give a compound of Formula (C-I).

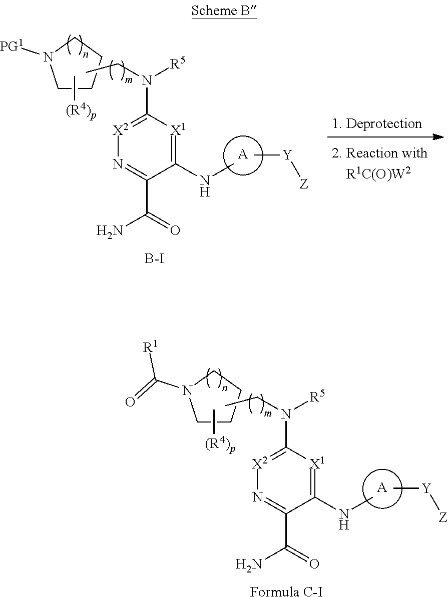

### Further Forms of Compounds

Compounds disclosed herein have a structure of any one of the Formulas described herein. It is understood that when reference is made to compounds described herein, it is meant to include compounds of any one of the Formulas described herein, as well as to all of the specific compounds that fall within the scope of these generic formulae, unless otherwise indicated.

The compounds described herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

Diasteromeric mixtures can be separated into their individual diastereomers on the basis of their physical chemical differences by methods known, for example, by chromatography and/or fractional crystallization. In one embodiment, enantiomers can be separated by chiral chromatographic columns. In other embodiments, enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

The methods and formulations described herein include the use of N-oxides, crystalline forms (also known as polymorphs), or pharmaceutically acceptable salts of compounds described herein, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of any one of the Formulas described herein in unoxidized form can be prepared from N-oxides of compounds of any one of Formula described herein by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985).

Prodrug forms of the herein described compounds, wherein the prodrug is metabolized in vivo to produce a derivative as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Sites on the aromatic ring portion of compounds of any of Formula (I) can be susceptible to various metabolic reactions, therefore incorporation of appropriate substituents on the aromatic ring structures, such as, by way of example only, halogens can reduce, minimize or eliminate this metabolic pathway.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the pharmaceutically acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof.

The salts are recovered by using at least one of the following techniques: filtration, precipitation with a non-solvent followed by filtration, evaporation of the solvent, or, in the case of aqueous solutions, lyophilization.

It should be understood that a reference to a salt, such as a pharmaceutically acceptable salt, includes the solvent addition forms or crystal forms thereof, particularly solvates or polymorphs. Solvates contain either stoichiometric or non-stoichiometric amounts of a solvent, and are often formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

The compounds described herein may be in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

### Pharmaceutical Composition/Formulation

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, compounds of any of Formula described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound described herein, such as, for example, a compound of any of Formula (A-I), (A-II), (A-IA)-(A-IH), (A-IIa), (A-IIb), (A-IIIa), (A-IIIb), (A-IVa)-(A-IVh), (A-Va)-(A-Vh), (A-VI), (A-VII) or (A-VIII), or Formula (B-I), (B-II), (B-IA), (B-IB), (B-IIa)-(B-IId), (B-IIIa)-(B-IIIe), (B-IVa)-(B-IVe) or (B-VIII), or Formula (C-I), (C-IA)-(C-IC), (C-IIa)-(C-IIe), (C-IIIa)-(C-IIIc), (C-VI), (C-IVa)-(C-IVd), (C-Va)-(C-Vd), (C-VIa)-(C-VId), or (C-VIIa)-(C-VIId) as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of any of Formula (described herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PHI 05, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, pig, or ng of therapeutic agent per ml, dl, or l of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. As used herein, the term “subject” is used to mean an animal, preferably a mammal, including a human or non-human. The terms patient and subject may be used interchangeably.

Moreover, the pharmaceutical compositions described herein, which include a compound of any one of Formula described herein can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of a compound described herein with one or more pharmaceutical excipients to form a bulk blend composition. When referring to these bulk blend compositions as homogeneous, it is meant that the particles of the compound described herein are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of the compound described herein. In one embodiment, some or all of the particles of the compound are coated. In another embodiment, some or all of the particles of the compound are microencapsulated. In still another embodiment, the particles of the compound are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound described herein from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet.

In some embodiments, the film coating can provide a delayed release of the compound described herein from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of the compound described herein inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compound described herein, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with compounds described herein, which sufficiently isolate the compound from other non-compatible excipients. Materials compatible with compounds described herein include those that delay the release of the compounds in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds described herein may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds described herein are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds described herein are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound described herein may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, the formulations are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated herein by reference. In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated herein by reference.

The pharmaceutical solid oral dosage forms, including formulations described herein, which include a compound described herein, can be further formulated to provide a controlled release of the compound. Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include a compound described herein are delivered using a pulsatile dosage form. A pulsatile dosage form is capable of providing one or more immediate release pulses at predetermined time points after a controlled lag time or at specific sites. Pulsatile dosage forms may be administered using a variety of pulsatile formulations known in the art. For example, such formulations include, but are not limited to, those described in U.S. Pat. Nos. 5,011,692, 5,017,381, 5,229,135, and 5,840,329, each of which is specifically incorporated by reference. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284, all of which are specifically incorporated herein by reference. In one embodiment, the controlled release dosage form is pulsatile release solid oral dosage form including at least two groups of particles, (i.e. multiparticulate) each containing the formulation described herein. The first group of particles provides a substantially immediate dose of the compound described herein upon ingestion by a mammal. The first group of particles can be either uncoated or include a coating and/or sealant. The second group of particles includes coated particles, which includes from about 2% to about 75%, from about 2.5% to about 70%, or from about 40% to about 70%, by weight of the total dose of the compound described herein in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the compound described herein.

Many other types of controlled release systems known to those of ordinary skill in the art and are suitable for use with the formulations described herein. Examples of such delivery systems include, e.g., polymer-based systems, such as polylactic and polyglycolic acid, polyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated herein by reference.

In some embodiments, pharmaceutical formulations are provided that include particles of the compounds described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition to the particles of compound described herein, the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80: PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate, triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated herein by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

### Intranasal Formulations

Intranasal formulations are known in the art and are described in, for example, U.S. Pat. Nos. 4,476,116, 5,116,817 and 6,391,452, each of which is specifically incorporated herein by reference. Formulations that include a compound described herein, which are prepared according to these and other techniques well-known in the art, may be prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation, the compounds described herein may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations that include compounds described herein may be administered using a variety of formulations known in the art. For example, such formulations include, but are not limited to, U.S. Pat. Nos. 4,229,447, 4,596,795, 4,755,386, and 5,739,136, each of which is specifically incorporated herein by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound described herein is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the compound described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated herein by reference in its entirety.

The transdermal dosage forms described herein may incorporate certain pharmaceutically acceptable excipients which are conventional in the art. In one embodiments, the transdermal formulations described herein include at least three components: (1) a compound described herein; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds described herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include a compound described herein suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The compounds described herein can be used in the preparation of medicaments for the inhibition of Btk or a homolog thereof, or for the treatment of diseases or conditions that would benefit, at least in part, from inhibition of Btk or a homolog thereof. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10/o %-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

The reversible or irreversible Btk inhibitor compositions described herein can also be used in combination with other well known therapeutic reagents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In certain instances, it may be appropriate to administer at least one reversible or irreversible Btk inhibitor compound described herein in combination with another therapeutic agent. By way of example only, if one of the side effects experienced by a patient upon receiving one of the reversible or irreversible Btk inhibitor compounds described herein is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is a compound of Formula (A-IA), (IIa) or (IIb) described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, within the first 6 hours of the onset of the symptoms, or within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound should be administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, between about 1 month to about 5 years, or from about 1 month to about 3 years.

Exemplary Therapeutic Agents for Use in Combination with a Reversible or Irreversible Btk Inhibitor Compound

Where the subject is suffering from or at risk of suffering from an autoimmune disease, an inflammatory disease, or an allergy disease, a reversible or irreversible Btk inhibitor compound can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

In the instance where the subject is suffering from or at risk of suffering from a B-cell proliferative disorder (e.g., plasma cell myeloma), the subject can be treated with a reversible or irreversible Btk inhibitor compound in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a reversible or irreversible Btk inhibitor compound include inhibitors of mitogen-activated protein kinase signaling, e.g., U0126, PD98059, PDI84352, PD0325901, ARRY-142886, SB239063, SP600125, BAY 43-9006, wortmannin, or LY294002; Syk inhibitors; mTOR inhibitors (e.g., everolimus and simrolimus); and antibodies (e.g., rituxan).

Other anti-cancer agents that can be employed in combination with a reversible or irreversible Btk inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a reversible or irreversible Btk inhibitor compound include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a reversible or irreversible Btk inhibitor compound include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).In some embodiments, the anti-cancer agent is a chemotherapeutic agent, analgesic, an immunotherapeutic agent, a targeted therapy, or a combination thereof. In some embodiments, the additional therapeutic agent is a B cell receptor pathway inhibitor. In some embodiments, the B cell receptor pathway inhibitor is a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, or a combination thereof. In some embodiments, the additional therapeutic agent is an antibody, B cell receptor signaling inhibitor, a PI3K inhibitor, an IAP inhibitor, an mTOR inhibitor, a radioimmunotherapeutic, a DNA damaging agent, a proteosome inhibitor, a histone deacetylase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In some embodiments, the additional therapeutic agent comprises an analgesic such as acetaminophen.

In some embodiments, the additional therapeutic agent comprises an agent selected from: an inhibitor of LYN, SYK, JAK, PI3K, PLCγ, MAPK, MEK or NFκB.

In some embodiments, the additional therapeutic agent comprises an agent selected from: bendamustine, bortezomib, lenalidomide, idelalisib (GS-1101), vorinostat, everolimus, panobinostat, temsirolimus, romidepsin, vorinostat, fludarabine, cyclophosphamide, mitoxantrone, pentostatine, prednisone, etopside, procarbazine, and thalidomide.

In some embodiments the additional therapeutic agent is bendamustine. In some embodiments, bortezomib is administered in combination with rituximab.

In some embodiments, the additional therapeutic agent is bortezomib. In some embodiments, bendamustine is administered in combination with rituximab.

In some embodiments, the additional therapeutic agent is lenalidomide. In some embodiments, lenalidomide is administered in combination with rituximab.

In some embodiments, the additional therapeutic agent is a multi-agent therapeutic regimen. In some embodiments the additional therapeutic agent comprises the HyperCVAD regimen (cyclophosphamide, vincristine, doxorubicin, dexamethasone alternating with methotrexate and cytarabine). In some embodiments, the HyperCVAD regimen is administered in combination with rituximab.

In some embodiments the additional therapeutic agent comprises the R-CHOP regiment (rituximab, cyclophosphamide, doxorubicin, vincristine, and prednisone).

In some embodiments the additional therapeutic agent comprises the FCR regimen (FCR (fludarabine, cyclophosphamide, rituximab).

In some embodiments the additional therapeutic agent comprises the FCMR regimen (fludarabine, cyclophosphamide, mitoxantrone, rituximab).

In some embodiments the additional therapeutic agent comprises the FMR regimen (fludarabine, mitoxantrone, rituximab).

In some embodiments the additional therapeutic agent comprises the PCR regimen (pentostatin, cyclophosphamide, rituximab).

In some embodiments the additional therapeutic agent comprises the PEPC regimen (prednisone, etoposide, procarbazine, cyclophosphamide).

In some embodiments the additional therapeutic agent comprises radioimmunotherapy with ⁹⁰Y-ibritumomab tiuxetan or ¹³¹-tositumomab.

In some embodiments, the additional therapeutic agent is an autologous stem cell transplant.

In some embodiments, the additional therapeutic agent is selected from: nitrogen mustards such as for example, bendamustine, chlorambucil, chlormethine, cyclophosphamide, ifosfamide, melphalan, prednimustine, trofosfamide; alkyl sulfonates like busulfan, mannosulfan, treosulfan; ethylene imines like carboquone, thiotepa, triaziquone; nitrosoureas like carmustine, fotemustine, lomustine, nimustine, ranimustine, semustine, streptozocin; epoxides such as for example, etoglucid; other alkylating agents such as for example dacarbazine, mitobronitol, pipobroman, temozolomide; folic acid analogues such as for example methotrexate, permetrexed, pralatrexate, raltitrexed; purine analogs such as for example cladribine, clofarabine, fludarabine, mercaptopurine, nelarabine, tioguanine; pyrimidine analogs such as for example azacitidine, capecitabine, carmofur, cytarabine, decitabine, fluorouracil, gemcitabine, tegafur; vinca alkaloids such as for example vinblastine, vincristine, vindesine, vinflunine, vinorelbine; podophyllotoxin derivatives such as for example etoposide, teniposide; colchicine derivatives such as for example demecolcine; taxanes such as for example docetaxel, paclitaxel, paclitaxel poliglumex; other plant alkaloids and natural products such as for example trabectedin; actinomycines such as for example dactinomycin; antracyclines such as for example aclarubicin, daunorubicin, doxorubicin, epirubicin, idarubicin, mitoxantrone, pirarubicin, valrubicin, zorubincin; other cytotoxic antibiotics such as for example bleomycin, ixabepilone, mitomycin, plicamycin; platinum compounds such as for example carboplatin, cisplatin, oxaliplatin, satraplatin; methylhydrazines such as for example procarbazine; sensitizers such as for example aminolevulinic acid, efaproxiral, methyl aminolevulinate, porfimer sodium, temoporfin; protein kinase inhibitors such as for example dasatinib, erlotinib, everolimus, gefitinib, imatinib, lapatinib, nilotinib, pazonanib, sorafenib, sunitinib, temsirolimus; other antineoplastic agents such as for example alitretinoin, altretamine, amzacrine, anagrelide, arsenic trioxide, asparaginase, bexarotene, bortezomib, celecoxib, denileukin diftitox, estramustine, hydroxycarbamide, irinotecan, lonidamine, masoprocol, miltefosein, mitoguazone, mitotane, oblimersen, pegaspargase, pentostatin, romidepsin, sitimagene ceradenovec, tiazofurine, topotecan, tretinoin, vorinostat; estrogens such as for example diethylstilbenol, ethinylestradiol, fosfestrol, polyestradiol phosphate; progestogens such as for example gestonorone, medroxyprogesterone, megestrol; gonadotropin releasing hormone analogs such as for example buserelin, goserelin, leuprorelin, triptorelin; anti-estrogens such as for example fulvestrant, tamoxifen, toremifene; anti-androgens such as for example bicalutamide, flutamide, nilutamide, enzyme inhibitors, aminoglutethimide, anastrozole, exemestane, formestane, letrozole, vorozole; other hormone antagonists such as for example abarelix, degarelix; immunostimulants such as for example histamine dihydrochloride, mifamurtide, pidotimod, plerixafor, roquinimex, thymopentin; immunosuppressants such as for example everolimus, gusperimus, leflunomide, mycophenolic acid, sirolimus; calcineurin inhibitors such as for example ciclosporin, tacrolimus; other immunosuppressants such as for example azathioprine, lenalidomide, methotrexate, thalidomide; and radiopharmaceuticals such as for example, iobenguane.

In some embodiments, the additional therapeutic agent is selected from: interferons, interleukins, tumor necrosis factors, growth factors, or the like.

In some embodiments, the additional therapeutic agent is selected from: ancestim, filgrastim, lenograstim, molgramostim, pegfilgrastim, sargramostim; interferons such as for example interferon α natural, interferon α-2a, interferon α-2b, interferon α-n1, interferon α-n1, interferon β natural, interferon β-1a, interferon β-1b, interferon γ, peginterferon α-2a, peginterferon α-2b; interleukins such as for example aldesleukin, oprelvekin; other immunostimulants such as for example BCG vaccine, glatiramer acetate, histamine dihydrochloride, immunocyanin, lentinan, melanoma vaccine, mifamurtide, pegademase, pidotimod, plerixafor, poly I:C, poly ICLC, roquinimex, tasonermin, thymopentin; immunosuppressants such as for example abatacept, abetimus, alefacept, antilymphocyte immunoglobulin (horse), antithymocyte immunoglobulin (rabbit), eculizumab, efalizumab, everolimus, gusperimus, leflunomide, muromab-CD3, mycophenolic acid, natalizumab, sirolimus; TNF a Inhibitors such as for example adalimumab, afelimomab, certolizumab pegol, etanercept, golimumab, infliximab; Interleukin Inhibitors such as for example anakinra, basiliximab, canakinumab, daclizumab, mepolizumab, rilonacept, tocilizumab, ustekinumab; calcineurin inhibitors such as for example ciclosporin, tacrolimus; other immunosuppressants such as for example azathioprine, lenalidomide, methotrexate, thalidomide.

In some embodiments, the additional therapeutic agent is selected from: adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, panitumumab, ranibizumab, tositumomab, trastuzumab, or the like, or a combination thereof.

In some embodiments, the additional therapeutic agent is selected from: monoclonal antibodies such as for example alemtuzumab, bevacizumab, catumaxomab, cetuximab, edrecolomab, gemtuzumab, panitumumab, trastuzumab; immunosuppressants, eculizumab, efalizumab, muromab-CD3, natalizumab; TNF alpha inhibitors such as for example adalimumab, afelimomab, certolizumab pegol, golimumab, infliximab; interleukin inhibitors, basiliximab, canakinumab, daclizumab, mepolizumab, tocilizumab, ustekinumab; radiopharmaceuticals, ibritumomab tiuxetan, tositumomab; others monoclonal antibodies such as for example abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, zalutumumab.

In some embodiments, the additional therapeutic agent is selected from: agents that affect the tumor micro-environment such as cellular signaling network (e.g. phosphatidylinositol 3-kinase (PI3K) signaling pathway, signaling from the B-cell receptor and the IgE receptor). In some embodiments, the additional therapeutic agent is a PI3K signaling inhibitor or a syc kinase inhibitor. In one embodiment, the syk inhibitor is R788. In another embodiment is a PKCγ inhibitor such as by way of example only, enzastaurin.

Examples of agents that affect the tumor micro-environment include P13K signaling inhibitor, syc kinase inhibitor, Protein Kinase Inhibitors such as for example dasatinib, erlotinib, everolimus, gefitinib, imatinib, lapatinib, nilotinib, pazonanib, sorafenib, sunitinib, temsirolimus; Other Angiogenesis Inhibitors such as for example GT-111, JI-101, R1530; Other Kinase Inhibitors such as for example AC220, AC480, ACE-041, AMG 900, AP24534, Arry-614, AT7519, AT9283, AV-951, axitinib, AZD1152, AZD7762, AZD8055, AZD8931, bafetinib, BAY 73-4506, BGJ398, BGT226, BI 811283, B16727, BIBF 1120, BIBW 2992, BMS-690154, BMS-777607, BMS-863233, BSK-461364, CAL-101, CEP-11981, CYC16, DCC-2036, dinaciclib, dovitinib lactate, E7050, EMD 1214063, ENMD-2076, fostamatinib disodium, GSK2256098, GSK690693, INCB18424, INNO-406, JNJ26483327, JX-594, KX2-391, linifanib, LY2603618, MGCD265, MK-0457, MK1496, MLN8054, MLN8237, MP470, NMS-1116354, NMS-1286937, ON 01919.Na, OSI-027, OSI-930, Btk inhibitor, PF-00562271, PF-02341066, PF-03814735, PF-04217903, PF-04554878, PF-04691502, PF-3758309, PHA-739358, PLC3397, progenipoietin, R547, R763, ramucirumab, regorafenib, RO5185426, SAR103168, SCH 727965, SGI-1176, SGX523, SNS-314, TAK-593, TAK-901, TK1258, TLN-232, TTP607, XL147, XL228, XL281RO5126766, XL4A18, XL765.

In some embodiments, the additional therapeutic agent is selected from: inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, the additional therapeutic agent is selected from: Adriamycin, Dactinomycin, Bleomycin, Vinblastine, Cisplatin, acivicin, aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rIL2), interferon α-2a; interferon α-2b; interferon α-n1; interferon α-n3; interferon β-1a; interferon γ-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In some embodiments, the additional therapeutic agent is selected from: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-such as for example growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, the additional therapeutic agent is selected from: alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

In some embodiments, the additional therapeutic agent is selected from: nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

In some embodiments, the additional therapeutic agent is selected from: agents which act by arresting cells in the G2-M phases due to stabilized microtubules, e.g., Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-I (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-IAABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NC1), Resverastatin phosphate sodium, BPR-OY-007 (National Health Research Institutes), and SSR-250411 (Sanofi).

Examples of natural products useful in combination with a reversible or irreversible Btk inhibitor compound include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon-α).

Examples of alkylating agents that can be employed in combination a reversible or irreversible Btk inhibitor compound include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists useful in combination with a reversible or irreversible Btk inhibitor compound include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with a reversible or irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, lnanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

In the instance where the subject is suffering from or at risk of suffering from a thromboembolic disorder (e.g., stroke), the subject can be treated with a reversible or irreversible Btk inhibitor compound in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048. In some embodiments, the additional anti-cancer agent that can be used in combination with the compounds described herein is a Bcl-2 inhibitor.

In some embodiments, the additional anti-cancer agent is an immune checkpoint inhibitor. In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of PD-L1, PD-1, CTLA-4, LAG3, or TIM3. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of PD-L2.

In some embodiments, a compound described herein is administered in combination with a CD20 inhibitor. Exemplary CD20 inhibitors include, but are not limited to, ibritumomab tiuxetan, ofatumumab, rituximab, tositumomab, and obinutuzumab.

In some embodiments, the additional anticancer agents used in combination with the compounds described herein include CDK4 inhibitors (e.g., palbociclib).

In some embodiments, the additional cancer agent is a proteosome inhibitor. In some embodiments, the proteasome inhibitor is selected from bortezomib or carfilzomib.

In some embodiments, the additional cancer agent that can be administered in combination with the compounds is an HDAC inhibitor. In some embodiments, the HDAC inhibitor is abexinostat or a salt thereof. In some embodiments, the abexinostat or a salt thereof is abexinostat HCl. In some embodiments, the abexinostat or a salt thereof is abexinostat tosylate.

In some embodiments, the additional cancer agent that can be administered in combination with the compounds is a MALT1 inhibitor, MCL-1 inhibitor, IDH1 inhibitor, TLR inhibitor, or PIM inhibitor.

In some embodiments, the additional anti-cancer agent that can be administered in combination with the compounds is an immunomodulatory agent Exemplary immunomodulatory agents include, but are not limited to, lenalidomide, thalidomide, and pomalidomide.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease, disorder, or condition that would benefit by inhibition of Btk, or in which Btk is a mediator or contributor to the symptoms or cause.

For example, the container(s) can include one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically may include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the pharmaceutical compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible pharmaceutical carrier can also be prepared, placed in an appropriate container, and labeled for treatment of an indicated condition.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information. The examples below as well as throughout the application, the following abbreviations have the following meanings. If not defined, the terms have their generally accepted meanings.
aq=aqueous
[(t-Bu)₃PH]BF₄=tri-tert-butylphosphonium tetrafluoroborate
t-BuOH=tertiary butanol
DCE=1,2-dichloroethane
DCM=dichloromethane
DIEA or DIPEA=N,N-diisopropylethylamine
DMAP=dimethylaminopyridine
DMF=dimethylformamide
DMSO=dimethylsulfoxide
ESI=electron spray ionization
EtOAc=ethyl acetate
g=gram
HCl=hydrogen chloride
HPLC=high performance liquid chromatography
¹H NMR=proton nuclear magnetic resonance
IPA=isopropyl alcohol
LC-MS=liquid chromatography mass spectroscopy
M=molar
MeCN=acetonitrile
MeOH=methanol
mg=milligram
min=minute
mL=milliliter
mM=millimolar
mmol=millimole
m.p.=melting point
MS=mass spectrometry
m/z=mass-to-charge ratio
N=normal
nM=nanomolar
nm=nanometer
Pd₂dba₃=tris(dibenzylideneacetone)dipalladium(0)
p.s.i.=pound per square inch
RT=room temperature
TEA=triethylamine
TFA=trifluoroacetic acid
TLC=thin layer chromatography
μL=microliter
μM=micromolar

### Example A-1: Synthesis of 3-{[4-(1-cyclopentylpiperidin-4-yl)phenyl]amino}-5-[(3R)-3-{[(pyridin-3-yl)carbamoyl]amino}piperidin-1-yl]pyrazine-2-carboxamide (1)

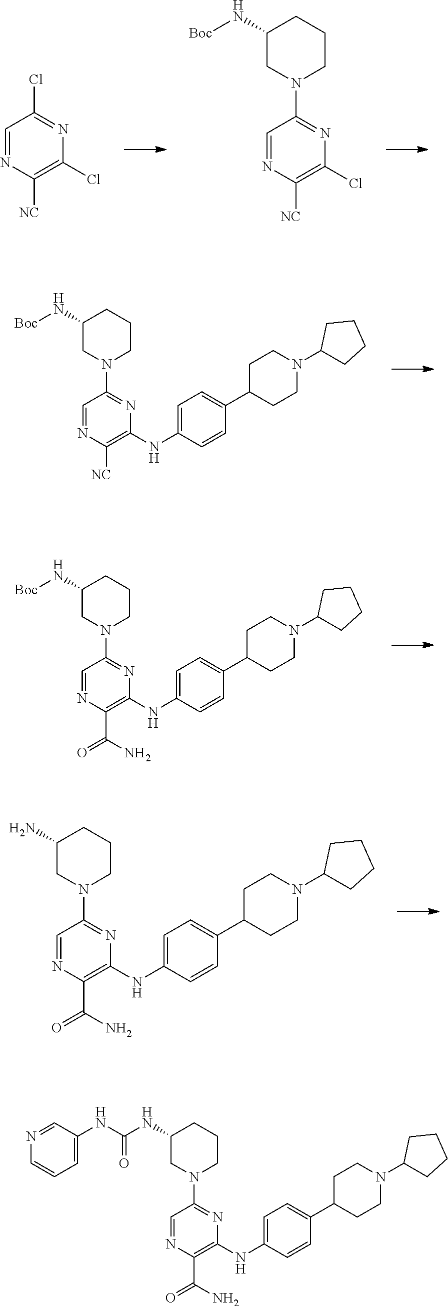 [SMILES: N#Cc1ncc(Cl)nc1Cl ; CC(C)(C)OC(=O)N[C@@H]1CCCN(c2cnc(C#N)c(Cl)n2)C1 ; CC(C)(C)OC(=O)N[C@@H]1CCCN(c2cnc(C#N)c(Nc3ccc(C4CCN(C5CCCC5)CC4)cc3)n2)C1 ; CC(C)(C)OC(=O)N[C@@H]1CCCN(c2cnc(C(N)=O)c(Nc3ccc(C4CCN(C5CCCC5)CC4)cc3)n2)C1 ; NC(=O)c1ncc(N2CCC[C@@H](N)C2)nc1Nc1ccc(C2CCN(C3CCCC3)CC2)cc1 ; NC(=O)c1ncc(N2CCC[C@@H](NC(=O)Nc3cccnc3)C2)nc1Nc1ccc(C2CCN(C3CCCC3)CC2)cc1]

To a solution of 3,5-dichloropyrazine-2-carbonitrile (7.00 g, 40.23 mmol) in DMF (50 mL) was added (R)-(3-BOC-amino)piperidine (8.64 g, 44.25 mmol) and DIPEA (14.0 mL, 5.74 mmol) in a dropwise manner. The mixture was stirred at room temperature for 90 min. The mixture was diluted with ethyl acetate (500 mL), washed with water (×2), dried, and concentrated in vacuo. The residue was purified by flash chromatography with 0 to 20% ethyl acetate in DCM to isolate tert-butyl N-[(3R)-1-(6-chloro-5-cyanopyrazin-2-yl)piperidin-3-yl]carbamate (12.59 g, 93% yield).

A mixture of tert-butyl N-[(3R)-1-(6-chloro-5-cyanopyrazin-2-yl)piperidin-3-yl]carbamate (1.447 g, 4.28 mmol), 4-(1-cyclopentylpiperidin-4-yl)aniline (1.254 g, 5.13 mmol), Pd(OAc)₂ (0.192 g, 0.85 mmol), BINAP (0.534 g, 0.85 mmol), fine powder Cs₂CO₃ (4.185 g, 12.84 mmol) in dioxane (60 mL) was degassed with a nitrogen stream for 10 min. The mixture was stirred in a nitrogen atmosphere at 115° C. for 1.5 h, then cooled to room temperature and Pd(OAc)₂ (96 mg, 0.1 eq) and BINAP (266 mg, 0.1 eq) were added. The mixture was degassed with nitrogen stream for 5 min and stirred at 115° C. overnight, and was then cooled, diluted with ethyl acetate, filtered through celite, and concentrated in vacuo. The residue was purified by flash chromatography with 0 to 10% MeOH in DCM to isolate tert-butyl N-[(3R)-1-(5-cyano-6-{[4-(1-cyclopentylpiperidin-4-yl)phenyl]amino} pyrazin-2-yl)piperidin-3-yl]carbamate (1.248 g, 53% yield).

To a solution of tert-butyl N-[(3R)-1-(5-cyano-6-{[4-(1-cyclopentylpiperidin-4-yl)phenyl]amino}pyrazin-2-yl)piperidin-3-yl]carbamate (1.995 g, 3.65 mmol) in MeOH (30 mL) and DMSO (3 mL) was added solid NaOH (400 mg) and 30% H₂O₂ (5 mL). The mixture was stirred at room temperature for 25 min, diluted with acetonitrile (20 mL), and ethyl acetate (300 mL) 10 min later. The organic phase was washed with water (×2), dried, and concentrated in vacuo to obtain tert-butyl N-[(3R)-1-(5-carbamoyl-6-{[4-(1-cyclopentylpiperidin-4-yl)phenyl]amino}pyrazin-2-yl)piperidin-3-yl]carbamate (2.843 g, quantitative yield). tert-butyl N-[(3R)-1-(5-carbamoyl-6-{[4-(1-cyclopentylpiperidin-4-yl)phenyl]amino}pyrazin-2-yl)piperidin-3-yl]carbamate was treated with 4N HCl in dioxane (60 mL) for 1 h. The mixture was concentrated in vacuo to dryness to obtain a residue that was passed through an SCX cartridge and eluted with ammonia in MeOH 1 N. The residue obtained was further purified by flash chromatography with 0 to 3% MeOH in DCM to afford 5-[(3R)-3-aminopiperidin-1-yl]-3-{[4-(1-cyclopentylpiperidin-4-yl)phenyl]amino}pyrazine-2-carboxamide (547 mg, 33% yield).

To a solution of 5-[(3R)-3-aminopiperidin-1-yl]-3-{[4-(1-cyclopentylpiperidin-4-yl)phenyl]amino}pyrazine-2-carboxamide (100 mg, 0.216 mmol) in THF (3 mL) was added 3-isocyanatopyridine (26 mg, 0.54 mmol). The mixture was stirred at room temperature for 2 h, then concentrated in vacuo. The residue was purified by flash chromatography (silica-NH) with 0 to 100/MeOH in DCM to afford 3-{[4-(1-cyclopentylpiperidin-4-yl)phenyl]amino}-5-[(3R)-3-{[(pyridin-3-yl)carbamoyl]amino)}piperidin-1-yl]pyrazine-2-carboxamide (1) as a orange-yellow solid (76.7 mg, 61% yield). MS found for C32H41N9O2 as (M+H)⁺ 584.5. ¹H NMR (500 MHz, DMSO) δ 11.25 (s, 1H), 8.61 (s, 1H), 8.50 (d, J=2.45 Hz, 1H), 8.12 (dd, J=4.65, 1.22 Hz, 1H), 7.99 (d, J=8.80 Hz, 1H), 7.75 (d, J=1.96 Hz, 1H), 7.67 (s, 1H), 7.52 (d, J=8.80 Hz, 2H), 7.39-7.24 (m, 2H), 7.11 (d, J=8.80 Hz, 2H), 6.47 (d, J=7.83 Hz, 1H), 4.29 (d, J=8.80 Hz, 1H), 3.91 (d, J=13.21 Hz, 1H), 3.75 (dd, J=8.07, 3.67 Hz, 1H), 3.47-3.37 (m, 1H), 3.25-3.12 (m, 1H), 2.97 (d, J=9.78 Hz, 2H), 2.48-2.40 (m, 1H), 2.28 (t, J=12.23 Hz, 1H), 1.97-1.29 (m, 18H).

### Example A-2: Synthesis of 3-{[4-(1-cyclopentylpiperidin-4-yl)phenyl]amino}-5-[(3R)-3-[(phenylcarbamoyl)amino]piperidin-1-yl]pyrazine-2-carboxamide hydrochloride (2)

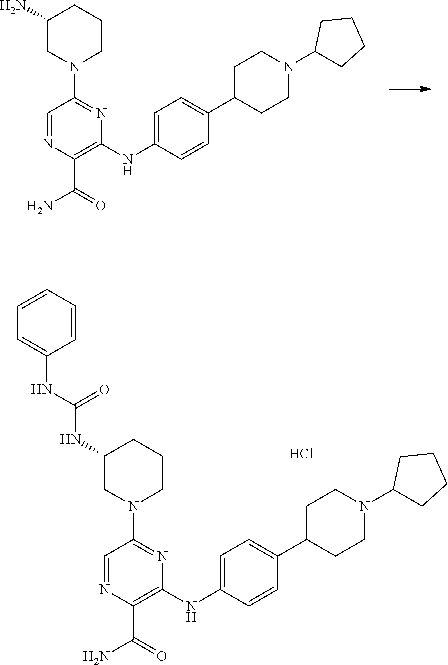 [SMILES: NC(=O)c1ncc(N2CCC[C@@H](N)C2)nc1Nc1ccc(C2CCN(C3CCCC3)CC2)cc1 ; NC(=O)c1ncc(N2CCC[C@@H](NC(=O)Nc3ccccc3)C2)nc1Nc1ccc(C2CCN(C3CCCC3)CC2)cc1 ; Cl]

In a similar manner as described in Example A-1, 3-{[4-(1-cyclopentylpiperidin-4-yl)phenyl]amino}-5-[(3R)-3-[(phenylcarbamoyl)amino]piperidin-1-yl]pyrazine-2-carboxamide hydrochloride (2) was prepared using phenyl isocyanate. MS found for C₃₃H₄₂N₈O₂ as (M+H)⁺ 583.3. ¹H NMR (400 MHz, CDCl₃) δ 11.27 (s, 1H), 8.70-8.45 (m, 1H), 7.79-7.71 (m, 1H), 7.66 (s, 1H), 7.54 (d, J=8.31 Hz, 2H), 7.44 (d, J=7.83 Hz, 2H), 7.34-7.20 (m, 3H), 7.15 (d, J=8.31 Hz, 2H), 6.88 (t, J=7.34 Hz, 1H), 6.62-6.31 (m, 1H), 4.52-3.64 (m, 5H), 2.98 (d, J=10.76 Hz, 2H), 2.59-2.22 (m, 2H), 1.95-1.30 (m, 18H).

### Example A-3: 5-[(3R)-3-[(cyclohexylcarbamoyl)amino]piperidin-1-yl]-3-{[4-(1-cyclopentylpiperidin-4-yl)phenyl]amino}pyrazine-2-carboxamide (3)

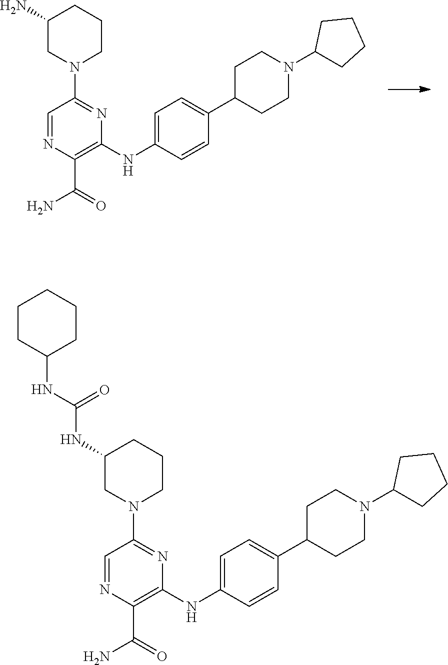 [SMILES: NC(=O)c1ncc(N2CCC[C@@H](N)C2)nc1Nc1ccc(C2CCN(C3CCCC3)CC2)cc1 ; NC(=O)c1ncc(N2CCC[C@@H](NC(=O)NC3CCCCC3)C2)nc1Nc1ccc(C2CCN(C3CCCC3)CC2)cc1]

In a similar manner as described in Example A-1, 5-[(3R)-3-[(cyclohexylcarbamoyl)amino]piperidin-1-yl]-3-{[4-(1-cyclopentylpiperidin-4-yl)phenyl]amino}pyrazine-2-carboxamide (3) was prepared using cyclohexyl isocyanate. MS found for C33H48N8O2 as (M+H)⁺ 589.3. ¹H NMR (500 MHz, DMSO) δ 11.25 (s, 1H), 7.72 (d, J=1.96 Hz, 1H), 7.62 (s, 1H), 7.53 (d, J=8.31 Hz, 2H), 7.30 (d, J=1.96 Hz, 1H), 7.17 (d, J=8.80 Hz, 2H), 5.83 (d, J=7.34 Hz, 1H), 5.71 (d, J=8.31 Hz, 1H), 4.39-4.16 (m, 1H), 3.97-3.83 (m, 1H), 3.65-3.53 (m, 1H), 3.47-3.34 (m, 2H), 3.02 (d, J=10.76 Hz, 3H), 2.48-2.32 (m, 2H), 2.01-1.00 (m, 28H).

### Example A-4: 3-{[4-(1-cyclopentylpiperidin-4-yl)phenyl]amino}-5-[(3R)-3-{[(3-methylphenyl)carbamoyl]amino}piperidin-1-yl]pyrazine-2-carboxamide hydrochloride (4)

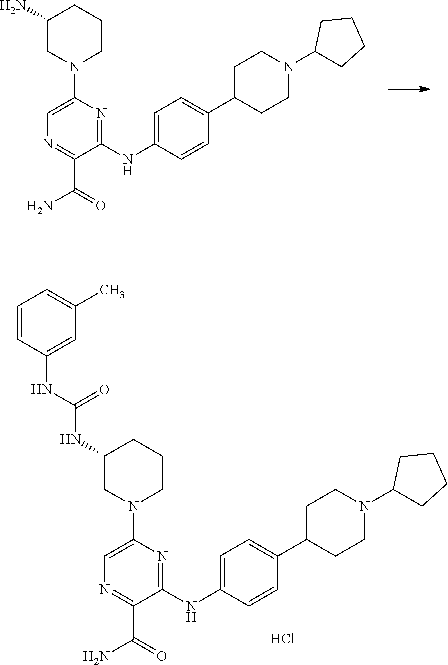 [SMILES: NC(=O)c1ncc(N2CCC[C@@H](N)C2)nc1Nc1ccc(C2CCN(C3CCCC3)CC2)cc1 ; Cc1cccc(NC(=O)N[C@@H]2CCCN(c3cnc(C(N)=O)c(Nc4ccc(C5CCN(C6CCCC6)CC5)cc4)n3)C2)c1 ; Cl]

In a similar manner as described in Example A-1, 3-{[4-(1-cyclopentylpiperidin-4-yl)phenyl]amino}-5-[(3R)-3-{[(3-methylphenyl)carbamoyl]amino}piperidin-1-yl]pyrazine-2-carboxamide hydrochloride (4) was prepared using m-tolyl isocyanate. MS found for C34H44N8O2 as (M+H)⁺ 597.1. ¹H NMR (500 MHz, DMSO) δ 11.24 (s, 1H), 9.3 (br. s., 1H), 8.33 (s, 1H), 7.74 (br. s., 1H), 7.66 (s, 1H), 7.52 (d, J=8.31 Hz, 2H), 7.32 (br. s., 1H), 7.28-7.18 (m, 2H), 7.17-7.06 (m, 3H), 6.71 (d, J=7.34 Hz, 1H), 6.29 (d, J=7.83 Hz, 1H), 4.35-4.16 (m, 1H), 3.90 (d, J=13.21 Hz, 1H), 3.77-3.64 (m, 1H), 3.41 (br. s., 1H), 3.24-3.15 (m, 1H), 2.97 (d, J=8.80 Hz, 2H), 2.48-2.41 (m, 1H), 2.33-2.20 (m, 4H), 1.99-1.27 (m, 18H).

### Example A-5: N-[(3R)-1-(5-carbamoyl-6-{[4-(1-cyclopentylpiperidin-4-yl)phenyl]amino}pyrazin-2-yl)piperidin-3-yl]-2,3-dihydro-1H-isoindole-2-carboxamide hydrochloride (5)

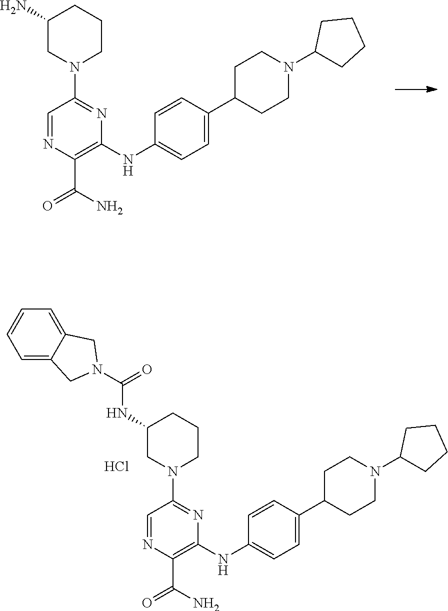 [SMILES: NC(=O)c1ncc(N2CCC[C@@H](N)C2)nc1Nc1ccc(C2CCN(C3CCCC3)CC2)cc1 ; NC(=O)c1ncc(N2CCC[C@@H](NC(=O)N3Cc4ccccc4C3)C2)nc1Nc1ccc(C2CCN(C3CCCC3)CC2)cc1 ; Cl]

To a solution of 2,3-dihydro-1H-isoindole (0.051 mL, 0.452 mmol) in DCM (4 mL) TEA (0.189 mL, 1.358 mmol) and triphosgene (63.9 mg, 0.215 mmol) were added at 0° C. The mixture was stirred at room temperature for 30 min. 5-[(3R)-3-aminopiperidin-1-yl]-3-[(5-methyl-1,2-thiazol-3-yl)amino]pyrazine-2-carboxamide (210 mg, 0.452 mmol) was added and the mixture was stirred overnight a room temperature. The mixture was diluted with DCM, washed with water (×2), dried, and concentrated in vacuo. The residue was purified by flash chromatography twice (silica-NH) with 0 to 5% MeOH in DCM and 0 to 2% MeOH in DCM to afford N-[(3R)-1-(5-carbamoyl-6-{[4-(1-cyclopentylpiperidin-4-yl)phenyl]amino}pyrazin-2-yl)piperidin-3-yl]-2,3-dihydro-1H-isoindole-2-carboxamide. The sample was treated with HCl in MeOH 2.5 M to obtain N-[(3R)-1-(5-carbamoyl-6-{[4-(1-cyclopentylpiperidin-4-yl)phenyl]amino}pyrazin-2-yl)piperidin-3-yl]-2,3-dihydro-1H-isoindole-2-carboxamide hydrochloride (5) (15.7 mg, 6% yield). MS found for C₃₅H₄₄N₈O₂ as (M+H)⁺ 609.1. ¹H NMR (500 MHz, DMSO) δ 11.43-11.18 (m, 1H), 9.22 (br. s., 1H), 7.77 (br. s., 1H), 7.69 (s, 1H), 7.61-7.53 (m, 2H), 7.39-7.24 (m, 5H), 7.15 (d, J=8.31 Hz, 2H), 6.25 (d, J=7.34 Hz, 1H), 4.79-4.54 (m, 4H), 4.49-4.13 (m, 2H), 3.82-3.23 (m, 3H), 3.18-2.64 (m, 6H), 2.12-1.40 (m, 16H).

### Example A-6: 3-{[4-(1-cyclopentylpiperidin-4-yl)phenyl]amino}-5-[(3R)-3-{[methyl(phenyl)carbamoyl]amino}piperidin-1-yl]pyrazine-2-carboxamide (6)

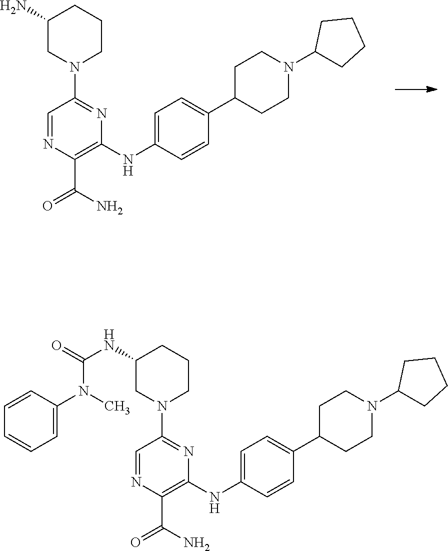 [SMILES: NC(=O)c1ncc(N2CCC[C@@H](N)C2)nc1Nc1ccc(C2CCN(C3CCCC3)CC2)cc1 ; CN(C(=O)N[C@@H]1CCCN(c2cnc(C(N)=O)c(Nc3ccc(C4CCN(C5CCCC5)CC4)cc3)n2)C1)c1ccccc1]

In a similar manner as described in Example A-5, 3-{[4-(1-cyclopentylpiperidin-4-yl)phenyl]amino}-5-[(3R)-3-{[methyl(phenyl)carbamoyl]amino}piperidin-1-yl]pyrazine-2-carboxamide (6) was prepared using N-methylaniline. MS found for C₃₄H₄₄N₈O₂ as (M+H)⁺ 597.3. ¹H NMR (500 MHz, DMSO) δ 11.23 (s, 1H), 7.75 (br. s., 1H), 7.62 (s, 1H), 7.49 (d, J=8.31 Hz, 2H), 7.36-7.28 (m, 3H), 7.20 (d, J=7.83 Hz, 2H), 7.15 (m, J=8.31 Hz, 3H), 5.77 (d, J=7.83 Hz, 1H), 4.21 (d, J=12.23 Hz, 1H), 4.04-3.92 (m, 1H), 3.75-3.62 (m, 1H), 3.17 (s, 3H), 3.21-3.08 (m, 2H), 3.05-2.97 (m, 2H), 2.41-2.33 (m, 1H), 2.00-1.91 (m, 2H), 1.87-1.27 (m, 17H).

### Example A-7: 3-{[4-(1-cyclopentylpiperidin-4-yl)phenyl]amino}-5-[(3R)-3-[4-(dimethylamino)benzamido]piperidin-1-yl]pyrazine-2-carboxamide hydrochloride (7)

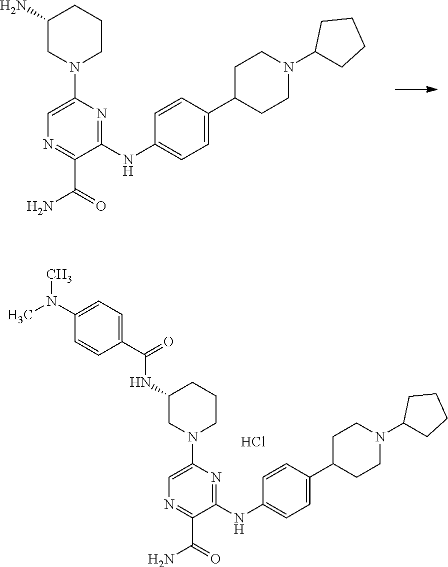 [SMILES: NC(=O)c1ncc(N2CCC[C@@H](N)C2)nc1Nc1ccc(C2CCN(C3CCCC3)CC2)cc1 ; CN(C)c1ccc(C(=O)N[C@@H]2CCCN(c3cnc(C(N)=O)c(Nc4ccc(C5CCN(C6CCCC6)CC5)cc4)n3)C2)cc1 ; Cl]

To a solution of 5-[(3R)-3-aminopiperidin-1-yl]-3-{[4-(1-cyclopentylpiperidin-4-yl)phenyl]amino}pyrazine-2-carboxamide (0.103 g, 0.22 mmol) in DMF (2 ml) 4-(dimethylamino)benzoyl chloride (0.052 g, 0.283 mmol) and DIPEA (0.115 mL, 0.66 mmol) were added. The mixture was stirred at room temperature for 1 h then concentrated in vacuo. The residue was purified by flash chromatography twice with 0 to 20% MeOH in DCM and with (silica-NH) 50% to 100% ethyl acetate in cyclohexane to afford 3-{[4-(1-cyclopentylpiperidin-4-yl)phenyl]amino}-5-[(3R)-3-[4-(dimethylamino)benzamido]piperidin-1-yl]pyrazine-2-carboxamide. It was treated with HCl in MeOH (3 mL, 1 N) to obtain 3-{[4-(1-cyclopentylpiperidin-4-yl)phenyl]amino}-5-[(3R)-3-[4-(dimethylamino)benzamido]piperidin-1-yl]pyrazine-2-carboxamide hydrochloride (7) (25 mg, 18% yield). MS found for C35H46N8O2 as (M+H)⁺ 611.1. ¹H NMR (500 MHz, DMSO) δ 11.23 (s, 1H), 9.56 (br. s., 1H), 8.03 (d, J=7.78 Hz, 1H), 7.85-7.73 (m, 3H), 7.70 (s, 1H), 7.56 (d, J=8.53 Hz, 2H), 7.32 (br. s., 1H), 7.13 (d, J=8.53 Hz, 2H), 6.73 (d, J=9.03 Hz, 2H), 4.44 (d, J=11.42 Hz, 1H), 4.21 (d, J=11.40 Hz, 1H), 3.96 (br. s., 1H), 3.66-3.38 (m, 2H), 3.16-2.93 (m, 10H), 2.85-2.60 (m, 1H), 2.19-1.42 (m, 17H).

### Example A-8: 3-{[4-(1-cyclopentylpiperidin-4-yl)phenyl]amino}-5-[(3R)-3-(3-methylbenzamido)piperidin-1-yl]pyrazine-2-carboxamide (8)

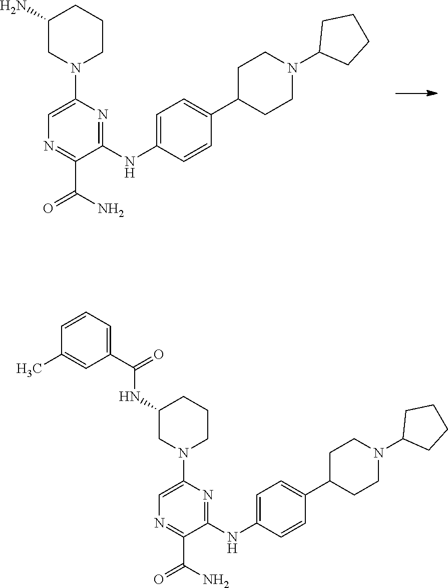 [SMILES: NC(=O)c1ncc(N2CCC[C@@H](N)C2)nc1Nc1ccc(C2CCN(C3CCCC3)CC2)cc1 ; Cc1cccc(C(=O)N[C@@H]2CCCN(c3cnc(C(N)=O)c(Nc4ccc(C5CCN(C6CCCC6)CC5)cc4)n3)C2)c1]

To a solution of 5-[(3R)-3-aminopiperidin-1-yl]-3-{[4-(1-cyclopentylpiperidin-4-yl)phenyl]amino}pyrazine-2-carboxamide (199 mg, 0.43 mmol) in DMF (2 mL), 3-methylbenzoic acid (89.3 mg, 0.65 mmol), DIPEA (0.25 mL, 1.26 mmol) and PyBOP (336.2 mg, 0.65 mmol) were added. The mixture was stirred at room temperature for 1 h, then concentrated and purified by flash chromatography from 0 to 5% MeOH in DCM to obtain a pale yellow residue. The residue was further purified by LC preparative chromatography to afford 3-{[4-(1-cyclopentylpiperidin-4-yl)phenyl]amino}-5-[(3R)-3-(3-methylbenzamido)piperidin-1-yl]pyrazine-2-carboxamide (8) (7 mg, 3% yield) as a yellowish solid. MS found for C34H43N7O2 as (M+H)⁺ 582.3. ¹H NMR (500 MHz, DMSO) δ 11.18 (s, 1H), 8.38 (d, J=7.58 Hz, 1H), 7.75 (s, 1H), 7.72-7.63 (m, 3H), 7.51 (d, J=8.56 Hz, 2H), 7.40-7.29 (m, 3H), 7.09 (d, J=8.56 Hz, 2H), 4.60-4.47 (m, 1H), 4.24-4.14 (m, 1H), 4.03-3.90 (m, 1H), 3.16-3.08 (m, 1H), 3.01-2.91 (m, 3H), 2.47-2.42 (m, 1H), 2.39 (s, 3H), 2.32 (s, 1H), 2.09-1.21 (m, 18H).

### Example A-9: 3-[(5-methyl-1,2-thiazol-5-yl)amino]-5-[(3R)-3-[(phenylcarbamoyl)amino]piperidin-1-yl]pyrazine-2-carboxamide (9)

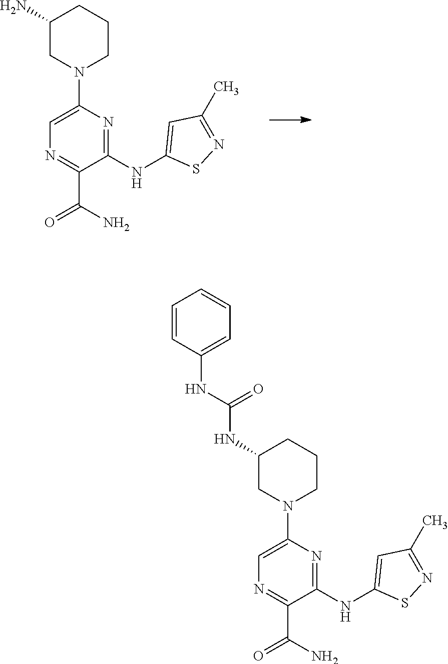 [SMILES: Cc1cc(Nc2nc(N3CCC[C@@H](N)C3)cnc2C(N)=O)sn1 ; Cc1cc(Nc2nc(N3CCC[C@@H](NC(=O)Nc4ccccc4)C3)cnc2C(N)=O)sn1]

In a similar manner as described in Example A-1, 3-[(5-methyl-1,2-thiazol-5-yl)amino]-5-[(3R)-3-[(phenylcarbamoyl)amino]piperidin-1-yl]pyrazine-2-carboxamide (9) was prepared using isocyanatobenzene. MS found for C21H24N8O2S as (M+H)⁺ 453.0. ¹H NMR (500 MHz, DMSO) δ 12.33 (br. s, 1H), 8.37 (s, 1H), 7.98-7.88 (m, 1H), 7.83 (s, 1H), 7.59-7.50 (m, 1H), 7.37 (d, J=7.83 Hz, 2H), 7.21 (t, J=8.07 Hz, 2H), 6.89 (t, J=7.34 Hz, 1H), 6.85 (s, 1H), 6.34 (d, J=7.34 Hz, 1H), 4.18-3.94 (m, 2H), 3.82-3.72 (m, 2H), 3.61-3.53 (m, 1H), 2.29 (s, 3H), 2.01-1.90 (m, 1H), 1.87-1.77 (m, 1H), 1.73-1.60 (m, 2H).

### Example A-10: 3-[(3-methyl-1,2-thiazol-5-yl)amino]-5-[(3R)-3-{[(3-methylphenyl)carbamoyl]amino}piperidin-1-yl]pyrazine-2-carboxamide (10)

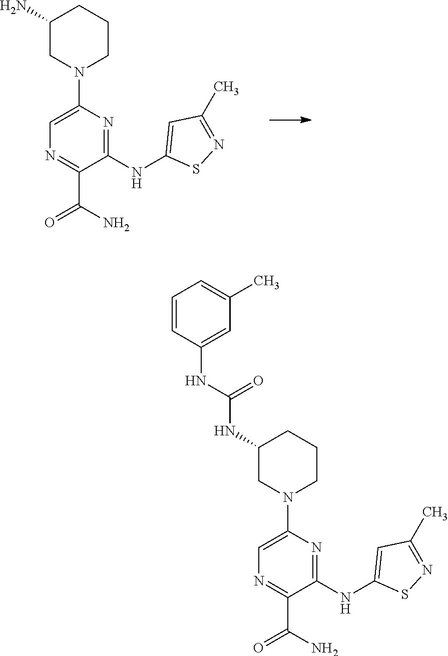 [SMILES: Cc1cc(Nc2nc(N3CCC[C@@H](N)C3)cnc2C(N)=O)sn1 ; Cc1cccc(NC(=O)N[C@@H]2CCCN(c3cnc(C(N)=O)c(Nc4cc(C)ns4)n3)C2)c1]

In a similar manner as described in Example A-1, 3-[(3-methyl-1,2-thiazol-5-yl)amino]-5-[(3R)-3-{[(3-methylphenyl)carbamoyl]amino}piperidin-1-yl]pyrazine-2-carboxamide (10) was prepared using m-tolyl isocyanate. MS found for C22H26N8O2S as (M+H)⁺ 467.0. ¹H NMR (500 MHz, DMSO) δ 12.29 (s, 1H), 8.28 (s, 1H), 7.90 (br. s., 1H), 7.81 (s, 1H), 7.53 (d, J=1.96 Hz, 1H), 7.22 (s, 1H), 7.16-7.01 (m, 2H), 6.84 (s, 1H), 6.70 (d, J=7.34 Hz, 1H), 6.30 (d, J=7.34 Hz, 1H), 4.19-4.06 (m, 1H), 4.04-3.91 (m, 1H), 3.82-3.69 (m, 2H), 3.54 (dd, J=13.21, 7.34 Hz, 1H), 2.23 (s, 3H), 2.28 (s, 3H), 1.94 (dt, J=7.70, 3.73 Hz, 1H), 1.85-1.74 (m, 1H), 1.72-1.49 (m, 2H)

### Example A-11: 3-[(5-methyl-1,2-thiazol-5-yl)amino]-5-[(3R)-3-{[(pyridin-3-yl)carbamoyl]amino}piperidin-1-yl]pyrazine-2-carboxamide (11)

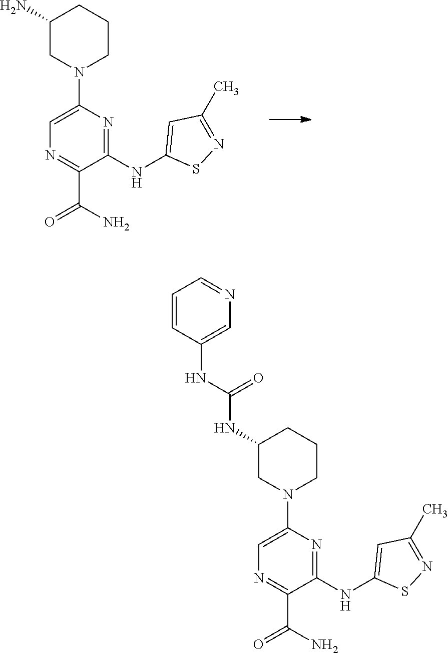 [SMILES: Cc1cc(Nc2nc(N3CCC[C@@H](N)C3)cnc2C(N)=O)sn1 ; Cc1cc(Nc2nc(N3CCC[C@@H](NC(=O)Nc4cccnc4)C3)cnc2C(N)=O)sn1]

In a similar manner as described in Example A-1, 3-[(5-methyl-1,2-thiazol-5-yl)amino]-5-[(3R)-3-{[(pyridin-3-yl)carbamoyl]amino}piperidin-1-yl]pyrazine-2-carboxamide (11) was prepared using pyridine-3-isocyanate. MS found for C20H23N9O2S as (M+H)⁺ 454.0. ¹H NMR (500 MHz, DMSO) δ 12.30 (s, 1H), 8.57 (s, 1H), 8.49 (d, J=2.45 Hz, 1H), 8.11 (dd, J=4.89, 1.47 Hz, 1H), 7.96-7.84 (m, 2H), 7.82 (s, 1H), 7.54 (br. s., 1H), 7.24 (dd, J=8.31, 4.89 Hz, 1H), 6.84 (s, 1H), 6.52 (d, J=7.83 Hz, 1H), 4.20-4.07 (m, 1H), 3.99 (br. s., 1H), 3.78 (br. s., 2H), 3.58 (dd, J=12.96, 7.58 Hz, 1H), 2.28 (s, 3H), 1.96 (br. s., 1H), 1.87-1.77 (m, 1H), 1.73-1.57 (m, 2H).

### Example A-12: 3-[(5-methyl-1,2-thiazol-5-yl)amino]-5-[(3R)-3-{[(5-methyl-1,3-thiazol-2-yl)carbamoyl]amino}piperidin-1-yl]pyrazine-2-carboxamide (12)

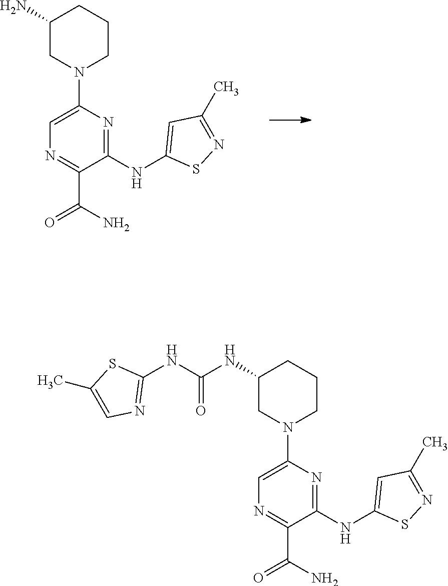

In a similar manner as described in Example A-5, 3-[(5-methyl-1,2-thiazol-5-yl)amino]-5-[(3R)-3-{[(5-methyl-1,3-thiazol-2-yl)carbamoyl]amino}piperidin-1-yl]pyrazine-2-carboxamide (12) was prepared using 2-amino-5-methylthiazole. MS found for C19H23N9O2S2 as (M+H)⁺ 473.9. ¹H NMR (500 MHz, Methanol) δ 7.77 (s, 1H), 6.79 (s, 1H), 6.72 (s, 1H), 4.16-3.80 (m, 5H), 2.36 (s, 3H), 2.31 (s, 3H), 2.15-1.75 (m, 4H).

### Example A-13: 5-[(3R)-3-[(cyclohexylcarbamoyl)amino]piperidin-1-yl]-3-[(3-methyl-1,2-thiazol-5-yl)amino]pyrazine-2-carboxamide (13)

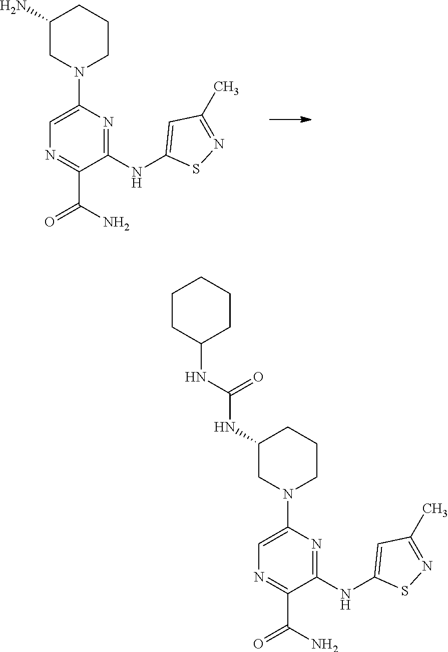 [SMILES: Cc1cc(Nc2nc(N3CCC[C@@H](N)C3)cnc2C(N)=O)sn1 ; Cc1cc(Nc2nc(N3CCC[C@@H](NC(=O)NC4CCCCC4)C3)cnc2C(N)=O)sn1]

In a similar manner as described in Example A-1, 5-[(3R)-3-[(cyclohexylcarbamoyl)amino]piperidin-1-yl]-3-[(3-methyl-1,2-thiazol-5-yl)amino]pyrazine-2-carboxamide (13) was prepared using cyclohexylisocyanate. MS found for C21H30N8O2S as (M+H)⁺ 459.0. ¹H NMR (500 MHz, DMSO) δ 12.21 (s, 1H), 7.81 (br. s., 1H), 7.69 (s, 1H), 7.46 (br. s., 1H), 6.78 (s, 1H), 5.78 (d, J=7.34 Hz, 1H), 5.63 (d, J=8.31 Hz, 1H), 4.00-3.82 (m, 2H), 3.77-3.62 (m, 1H), 3.61-3.51 (m, 1H), 3.43 (dd, J=12.72, 7.34 Hz, 1H), 3.37-3.18 (m, 1H), 2.23 (s, 3H), 1.89-1.37 (m, 9H), 1.27-0.89 (m, 5H).

### Example A-14: 5-[(3R)-3-aminopiperidin-1-yl]-3-[(5-methyl-1,2-thiazol-5-yl)amino]pyrazine-2-carboxamide (14)

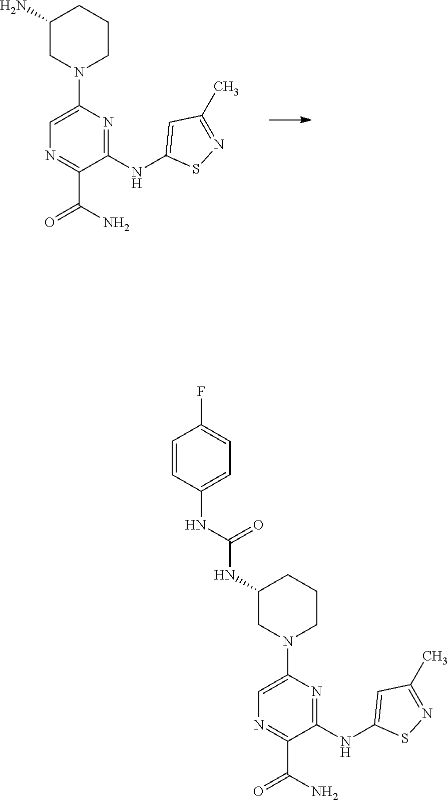 [SMILES: Cc1cc(Nc2nc(N3CCC[C@@H](N)C3)cnc2C(N)=O)sn1 ; Cc1cc(Nc2nc(N3CCC[C@@H](NC(=O)Nc4ccc(F)cc4)C3)cnc2C(N)=O)sn1]

In a similar manner as described in Example A-1, 5-[(3R)-3-aminopiperidin-1-yl]-3-[(5-methyl-1,2-thiazol-5-yl)amino]pyrazine-2-carboxamide (14) was prepared using 4-fluorophenyl isocyanate. MS found for C21H23FN8O2S as (M+H)⁺ 471.0. ¹H NMR (500 MHz, DMSO) δ 12.29 (br. s, 1H), 8.45-8.35 (m, 1H), 7.94-7.86 (m, 1H), 7.84-7.78 (m, 1H), 7.60-7.49 (m, 1H), 7.41-7.31 (m, 2H), 7.10-6.98 (m, 2H), 6.87-6.81 (m, 1H), 6.31 (d, J=7.43 Hz, 1H), 4.17-4.06 (m, 1H), 4.05-3.95 (m, 1H), 3.83-3.68 (m, 2H), 3.61-3.50 (m, 1H), 2.29 (s, 3H), 2.02-1.89 (m, 1H), 1.88-1.74 (m, 1H), 1.73-1.56 (m, 2H).

### Example A-15: 5-[(3R)-3-{[(4-methoxyphenyl)carbamoyl]amino}piperidin-1-yl]-3-[(3-methyl-1,2-thiazol-5-yl)amino]pyrazine-2-carboxamide (15)

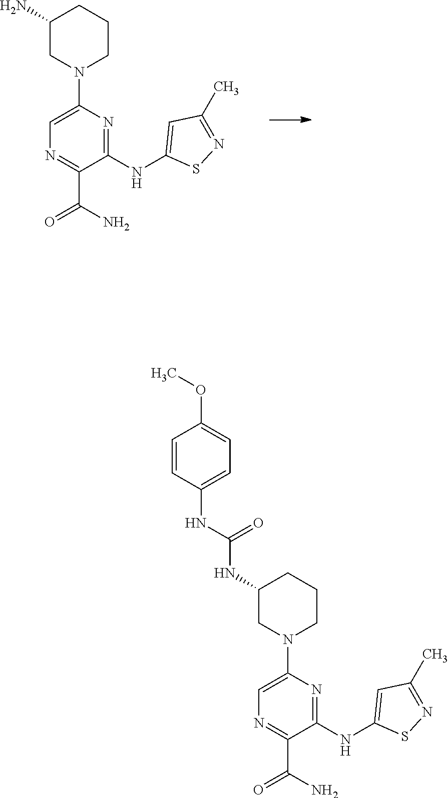 [SMILES: Cc1cc(Nc2nc(N3CCC[C@@H](N)C3)cnc2C(N)=O)sn1 ; COc1ccc(NC(=O)N[C@@H]2CCCN(c3cnc(C(N)=O)c(Nc4cc(C)ns4)n3)C2)cc1]

In a similar manner as described in Example A-1, 5-[(3R)-3-{[(4-methoxyphenyl)carbamoyl]amino}piperidin-1-yl]-3-[(3-methyl-1,2-thiazol-5-yl)amino]pyrazine-2-carboxamide (15) was prepared using 4-methoxyphenyl isocyanate. MS found for C22H26N8O3S as (M+H)⁺ 483.0. ¹H NMR (500 MHz, DMSO) δ 12.29 (s, 1H), 8.16 (s, 1H), 7.90 (br. s., 1H), 7.81 (s, 1H), 7.53 (br. s., 1H), 7.26 (d, J=9.00 Hz, 2H), 6.84 (s, 1H), 6.80 (d, J=9.00 Hz, 2H), 6.21 (d, J=7.43 Hz, 1H), 4.17-3.95 (m, 2H), 3.81-3.63 (m, 5H), 3.60-3.50 (m, 1H), 2.29 (s, 3H), 2.01-1.88 (m, 1H), 1.88-1.73 (m, 1H), 1.73-1.55 (m, 2H).

### Example A-16: 5-[(3R)-3-{[(3-methoxyphenyl)carbamoyl]amino}piperidin-1-yl]-3-[(3-methyl-1,2-thiazol-5-yl)amino]pyrazine-2-carboxamide (16)

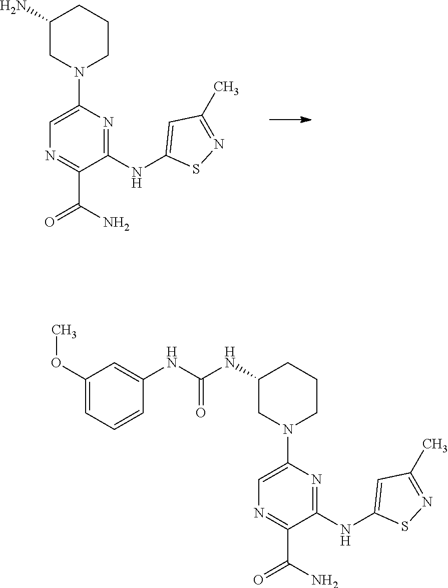 [SMILES: Cc1cc(Nc2nc(N3CCC[C@@H](N)C3)cnc2C(N)=O)sn1 ; COc1cccc(NC(=O)N[C@@H]2CCCN(c3cnc(C(N)=O)c(Nc4cc(C)ns4)n3)C2)c1]

In a similar manner as described in Example A-1, 5-[(3R)-3-{[(3-methoxyphenyl)carbamoyl]amino)}piperidin-1-yl]-3-[(3-methyl-1,2-thiazol-5-yl)amino]pyrazine-2-carboxamide (16) was prepared using 4-methoxyphenyl isocyanate. MS found for C22H26N8O3S as (M+H)⁺ 483.0. ¹H NMR (500 MHz, DMSO) δ 12.29 (br. s, 1H), 8.36 (br. s, 1H), 7.94-7.85 (m, 1H), 7.83-7.78 (m, 1H), 7.57-7.45 (m, 1H), 7.14-7.11 (m, 1H), 7.08 (t, J=8.22 Hz, 1H), 6.83 (s, 1H), 6.80 (d, J=8.22 Hz, 1H), 6.46 (dd, J=8.22, 1.96 Hz, 1H), 6.31 (d, J=7.43 Hz, 1H), 4.15-4.04 (m, 1H), 4.02-3.91- (m, 1H), 3.83-3.71 (m, 2H), 3.69 (s, 3H), 3.62-3.49 (m, 1H), 2.27 (s, 3H), 2.00-1.87 (m, 1H), 1.85-1.76 (m, 1H), 1.72-1.56 (m, 2H)

### Example A-17: N-[(3R)-1-(5-carbamoyl-6-[(3-methyl-1,2-thiazol-5-yl)amino]pyrazin-2-yl)piperidin-3-yl]-2,3-dihydro-1H-isoindole-2-carboxamide (17)

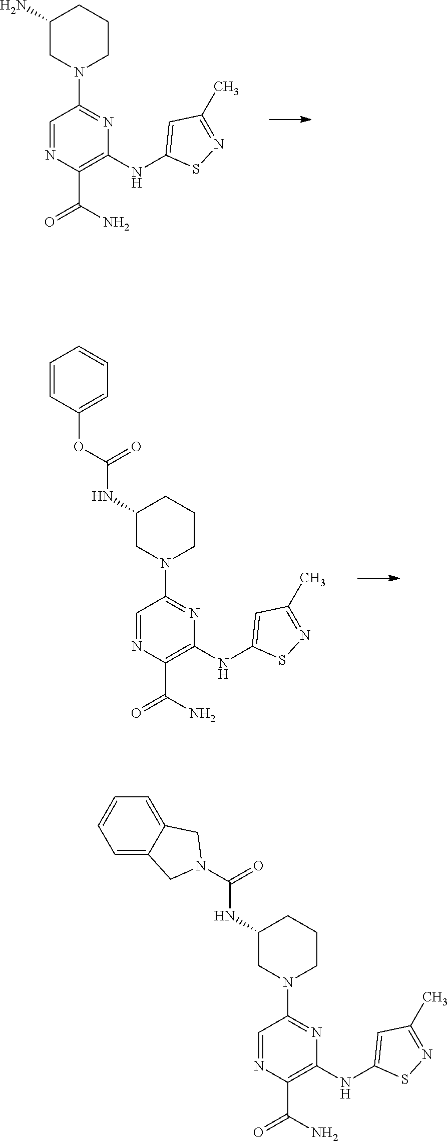 [SMILES: Cc1cc(Nc2nc(N3CCC[C@@H](N)C3)cnc2C(N)=O)sn1 ; Cc1cc(Nc2nc(N3CCC[C@@H](NC(=O)Oc4ccccc4)C3)cnc2C(N)=O)sn1 ; Cc1cc(Nc2nc(N3CCC[C@@H](NC(=O)N4Cc5ccccc5C4)C3)cnc2C(N)=O)sn1]

5-[(3R)-3-aminopiperidin-1-yl]-3-[(5-methyl-1,2-thiazol-5-yl)amino]pyrazine-2-carboxamide (150 mg, 0.45 mmol) was dissolved in DCM (3 mL) then TEA (0.125 mL, 0.9 mmol) and phenyl chloroformate (0.056 ml, 0.45 mmol) were added. The mixture was stirred for 10 min at room temperature then methanol (5 mL) was added. The solvent was removed and the residue was dissolved in DMF (2 mL), TEA (0.188 mL, 1.35 mmol) was added, followed by isoindoline (0.9 mmol). The mixture was heated at 50° C. overnight then it was charged on a SCX cartridge and, after several washes with methanol, was eluted with ammonia in MeOH 1N. The material was dried under vacuum and purified by flash chromatography 50 to 95% DCM in cyclohexane to afford N-[(3R)-1-{5-carbamoyl-6-[(3-methyl-1,2-thiazol-5-yl)amino]pyrazin-2-yl}piperidin-3-yl]-2,3-dihydro-1H-isoindole-2-carboxamide (38.4 mg, 18% yield). MS found for C23H26N8O2S as (M+H)⁺ 479.0. ¹H NMR (500 MHz, DMSO) δ 12.26 (br. s, 1H), 7.83 (s, 1H), 7.72-7.57 (m, 1H), 7.34-7.24 (m, 4H), 6.75 (s, 1H), 6.69-6.60 (m, 1H), 5.72-5.63 (m, 1H), 4.75-4.57 (m, 4H), 4.52-4.45 (m, 1H), 4.45-4.37 (m, 1H), 3.94-3.84 (m, 1H), 3.52-3.44 (m, 1H), 3.43-3.36 (m, 1H), 2.31 (s, 3H), 2.16-2.05 (m, 1H), 2.02-1.94 (m, 1H), 1.88-1.69 (m, 2H).

### Example A-18: N-[(3R)-1-{6-carbamoyl-5-[(3-methyl-1,2-thiazol-5-yl)amino]pyridin-3-yl}piperidin-3-yl]-2,3-dihydro-1H-isoindole-2-carboxamide (18)

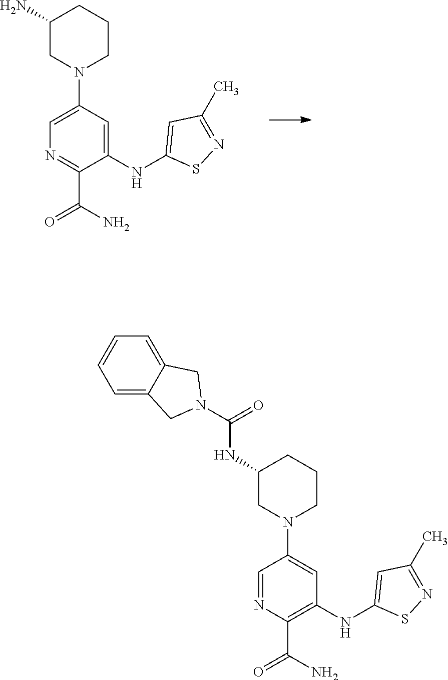 [SMILES: Cc1cc(Nc2cc(N3CCC[C@@H](N)C3)cnc2C(N)=O)sn1 ; Cc1cc(Nc2cc(N3CCC[C@@H](NC(=O)N4Cc5ccccc5C4)C3)cnc2C(N)=O)sn1]

In a similar manner as described in Example A-5, N-[(3R)-1-(6-carbamoyl-5-[(3-methyl-1,2-thiazol-5-yl)amino]pyridin-3-yl) piperidin-3-yl]-2,3-dihydro-1H-isoindole-2-carboxamide (18) was prepared using Isoindoline and triphosgene. MS found for C24H27N7O2S as (M+H)⁺ 478.1. ¹H NMR (500 MHz, DMSO) δ 12.02 (s, 1H), 8.02 (br. s., 1H), 7.94 (d, J=2.20 Hz, 1H), 7.55 (br. s., 1H), 7.38-7.22 (m, 4H), 6.96 (d, J=2.20 Hz, 1H), 6.89 (s, 1H), 6.21 (d, J=7.14 Hz, 1H), 4.68-4.52 (m, 4H), 3.97 (d, J=12.70 Hz, 1H), 3.86 (d, J=11.00 Hz, 1H), 3.73-3.61 (m, 1H), 3.02 (t, J=11.05 Hz, 1H), 2.90 (dd, J=12.69, 10.09 Hz, 1H), 2.34 (s, 3H), 1.98-1.89 (m, 1H), 1.86-1.76 (m, 1H), 1.72-1.48 (m, 2H).

### Example A-19: 5-[(3R)-3-benzamidopiperidin-1-yl]-3-[(3-methyl-1,2-thiazol-5-yl)amino]pyrazine-2-carboxamide (19)

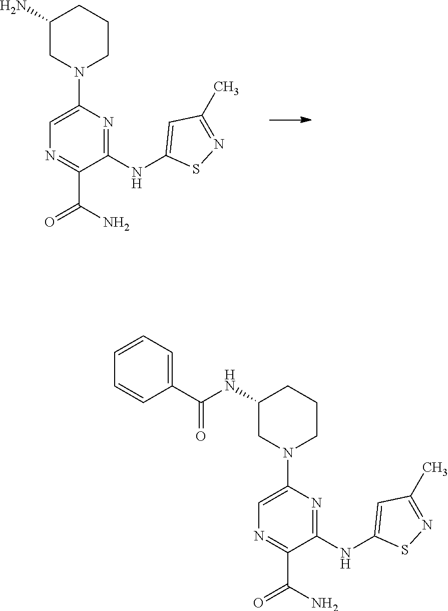 [SMILES: Cc1cc(Nc2nc(N3CCC[C@@H](N)C3)cnc2C(N)=O)sn1 ; Cc1cc(Nc2nc(N3CCC[C@@H](NC(=O)c4ccccc4)C3)cnc2C(N)=O)sn1]

In similar manner as described in Example A-7, 5-[(3R)-3-benzamidopiperidin-1-yl]-3-[(3-methyl-1,2-thiazol-5-yl)amino]pyrazine-2-carboxamide (19) was prepared using benzoyl chloride. MS found for C21H23N7O2S as (M+H)⁺ 438.0. ¹H NMR (400 MHz, DMSO) δ 12.26 (br. s, 1H), 8.45 (d, J=7.43 Hz, 1H), 7.95-7.89 (m, 1H), 7.87-7.79 (m, 3H), 7.58-7.41 (m, 4H), 6.84 (s, 1H), 4.52-4.35 (m, 2H), 4.06-3.92 (m, 1H), 3.42-3.21 (m, 2H), 2.28 (s, 3H), 2.06-1.97 (m, 1H), 1.97-1.88 (m, 1H), 1.8-1.70 (m, 1H), 1.69-1.57 (m, 1H).

### Example A-20: 5-[(3R)-3-(4-fluorobenzamido)piperidin-1-yl]-3-[(3-methyl-1,2-thiazol-5-yl)amino]pyrazine-2-carboxamide (20)

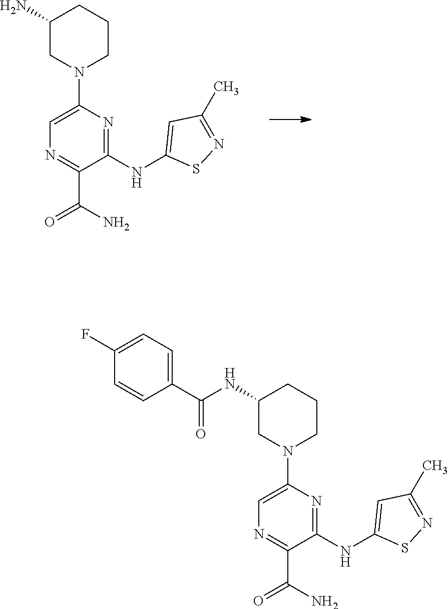 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(F)cc4)C3)cnc2C(N)=O)sn1]

In similar manner as described in Example A-7, 5-[(3R)-3-(4-fluorobenzamido)piperidin-1-yl]-3-[(3-methyl-1,2-thiazol-5-yl)amino]pyrazine-2-carboxamide (20) was prepared using 4-fluorobenzoyl chloride. MS found for C21H22FN7O2S as (M+H)⁺ 455.9. ¹H NMR (500 MHz, DMSO) δ 12.29 (br. s, 1H), 8.48 (d, J=7.34 Hz, 1H), 7.92-7.90 (m, 3H), 7.83 (s, 1H), 7.59-7.50 (m, 1H), 7.30 (t, J=9.05 Hz, 2H), 6.85 (s, 1H), 4.54-4.43 (m, 1H), 4.43-4.35 (m, 1H), 4.02-3.91 (m, 1H), 3.42-3.18 (m, 2H), 2.27 (s, 3H), 2.05-1.96 (m, 1H), 1.96-1.88 (m, 1H), 1.80-1.69 (m, 1H), 1.69-1.56 (m, 1H).

### Example A-21: 5-[(3R)-3-[4-(dimethylamino)benzamido]piperidin-1-yl]-3-[(3-methyl-1,2-thiazol-5-yl)amino]pyrazine-2-carboxamide (21)

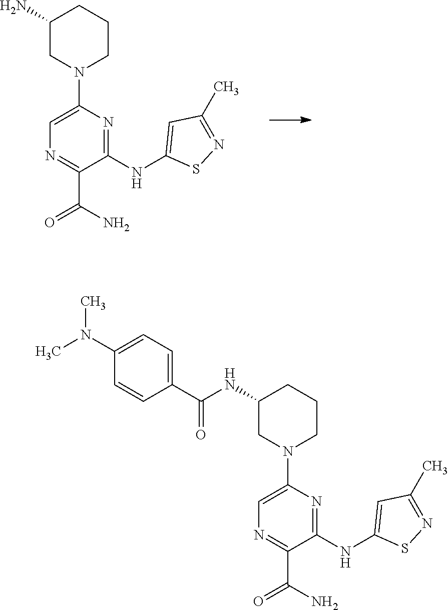

In similar manner as described in Example A-7, 5-[(3R)-3-[4-(dimethylamino)benzamido]piperidin-1-yl]-3-[(3-methyl-1,2-thiazol-5-yl)amino]pyrazine-2-carboxamide (21) was prepared using 4-(dimethylamino)benzoyl chloride. MS found for C23H28N8O2S as (M+H)⁺ 481.4. ¹H NMR (400 MHz, DMSO) δ 12.28 (s, 1H), 8.05 (d, J=7.28 Hz, 1H), 7.94-7.87 (m, 1H), 7.84 (s, 1H), 7.73 (d, J=9.03 Hz, 2H), 7.59-7.48 (m, 1H), 6.84 (s, 1H), 6.70 (d, J=9.03 Hz, 2H), 4.55-4.37 (m, 2H), 3.85- (m, 1H), 3.28-3.15 (m, 2H), 2.97 (s, 6H), 2.28 (s, 3H), −2.03-1.87 (m, 2H), 1.82-1.69 (m, 1H), 1.68-1.54 (m, 1H).

### Example A-22: 5-[(3R)-3-(3-fluorobenzamido)piperidin-1-yl]-3-[(3-methyl-1,2-thiazol-5-yl)amino]pyrazine-2-carboxamide (22)

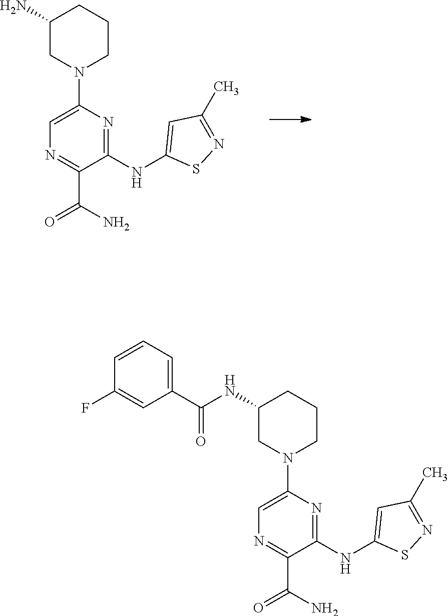 [SMILES: Cc1cc(Nc2nc(N3CCC[C@@H](N)C3)cnc2C(N)=O)sn1 ; Cc1cc(Nc2nc(N3CCC[C@@H](NC(=O)c4cccc(F)c4)C3)cnc2C(N)=O)sn1]

In similar manner as described in Example A-8, 5-[(3R)-3-(3-fluorobenzamido)piperidin-1-yl]-3-[(3-methyl-1,2-thiazol-5-yl)amino]pyrazine-2-carboxamide (22) was prepared using 3-fluorobenzoic acid. MS found for C21H22FN7O2S as (M+H)⁺ 456.1. ¹H NMR (500 MHz, DMSO) δ 12.30 (s, 1H), 8.55 (d, J=7.34 Hz, 1H), 7.98-7.89 (m, 1H), 7.85 (s, 1H), 7.69 (d, J=7.83 Hz, 1H), 7.63 (d, J=9.29 Hz, 1H), 7.57-7.48 (m, 2H), 7.39 (td, J=8.56, 1.96 Hz, 1H), 6.85 (s, 1H), 4.59-4.26 (m, 2H), 4.07-3.88 (m, 1H), 3.41-3.21 (m, 2H), 2.28 (s, 3H), 2.06-1.97 (m, 1H), 1.98-1.89 (m, 1H), 1.80-1.68 (m, 1H), 1.68-1.58 (m, 1H).

### Example A-23: 5-[(3R)-3-(2-fluorobenzamido)piperidin-1-yl]-3-[(3-methyl-1,2-thiazol-5-yl)amino]pyrazine-2-carboxamide (23)

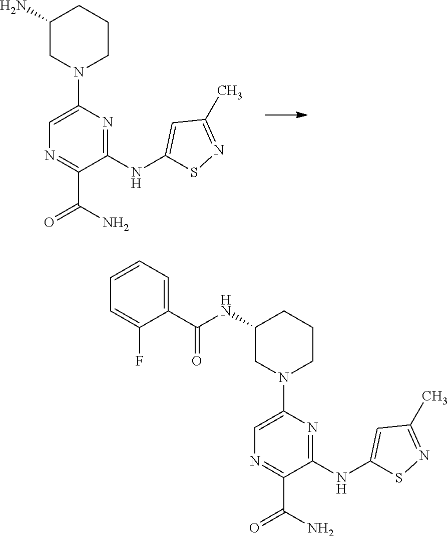 [SMILES: Cc1cc(Nc2nc(N3CCC[C@@H](N)C3)cnc2C(N)=O)sn1 ; Cc1cc(Nc2nc(N3CCC[C@@H](NC(=O)c4ccccc4F)C3)cnc2C(N)=O)sn1]

In similar manner as described in Example A-7, 5-[(3R)-3-(2-fluorobenzamido)piperidin-1-yl]-3-[(3-methyl-1,2-thiazol-5-yl)amino]pyrazine-2-carboxamide (23) was prepared using 2-fluorobenzoyl chloride. MS found for C21H22FN7O2S as (M+H)⁺ 456.3. ¹H NMR (500 MHz, DMSO) δ 12.31 (s, 1H), 8.45 (d, J=7.34 Hz, 1H), 7.95-7.88 (m, 1H), 7.83 (s, 1H), 7.57-7.21 (m, 5H), 6.86 (s, 1H), 4.44-4.13 (m, 2H), 3.54-3.37 (m, 2H), 4.05-3.33 (m, 1H), 2.29 (s, 3H), 2.07-1.86 (m, 2H), 1.79-1.57 (m, 2H).

### Example A-24: 5-[(3R)-3-(2-fluoro-4-methylbenzamido)piperidin-1-yl]-3-[(3-methyl-1,2-thiazol-5-yl)amino]pyrazine-2-carboxamide (24)

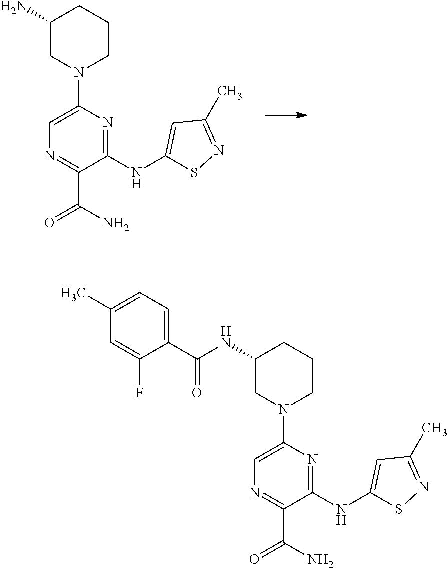

In similar manner as described in Example A-8, 5-[(3R)-3-(2-fluoro-4-methylbenzamido)piperidin-1-yl]-3-[(3-methyl-1,2-thiazol-5-yl)amino]pyrazine-2-carboxamide (24) was prepared using 2-fluoro-4-methylbenzoic acid. MS found for C22H24FN7O2S as (M+H)⁺ 470.0. ¹H NMR (500 MHz, DMSO) δ 12.31 (s, 1H), 8.29 (d, J=6.85 Hz, 1H), 7.91 (br. s., 1H), 7.83 (s, 1H), 7.54 (br. s., 1H), 7.45 (t, J=7.58 Hz, 1H), 7.14-7.04 (m, 2H), 6.86 (s, 1H), 4.45-4.15 (m, 2H), 4.07-3.88 (m, 1H), 3.57-3.38 (m, 2H), 2.34 (s, 3H), 2.29 (s, 3H), 2.04-1.95 (m, 1H), 1.95-1.86 (m, 1H), 1.78-1.57 (m, 2H).

### Example A-25: 5-[(3R)-3-(4-aminobenzamido)piperidin-1-yl]-3-[(3-methyl-1,2-thiazol-5-yl)amino]pyrazine-2-carboxamide (25)

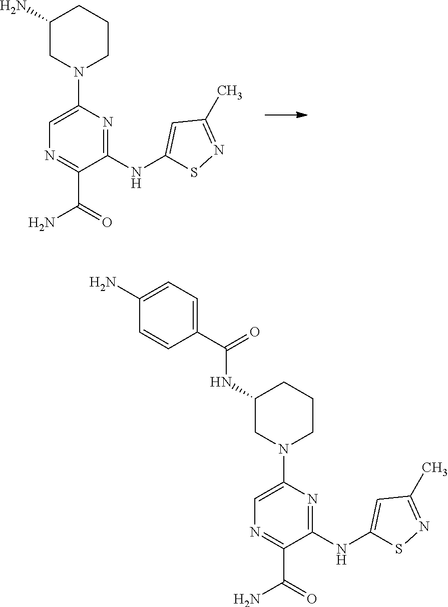 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(N)cc4)C3)cnc2C(N)=O)sn1]

In similar manner as described in Example A-8, 5-[(3R)-3-(4-aminobenzamido)piperidin-1-yl]-3-[(3-methyl-1,2-thiazol-5-yl)amino]pyrazine-2-carboxamide (25) was prepared using 4-aminobenzoic acid. MS found for C21H24N8O2S as (M+H)⁺ 453.4. ¹H NMR (500 MHz, DMSO) δ 12.32 (s, 1H), 8.15 (d, J=6.85 Hz, 1H), 7.92 (br. s., 1H), 7.85 (s, 1H), 7.69 (d, J=8.31 Hz, 2H), 7.55 (br. s., 1H), 6.93-6.73 (m, 3H), 4.53-4.35 (m, 2H), 4.02-3.84 (m, 1H), 3.36-3.15 (m, 2H), 2.29 (s, 3H), 2.05-1.83 (m, 2H), 1.81-1.53 (m, 2H).

### Example A-26: 3-[(3-methyl-1,2-thiazol-5-yl)amino]-5-[(3R)-3-[4-(pyrrolidin-1-yl)benzamido]piperidin-1-yl]pyrazine-2-carboxamide (26)

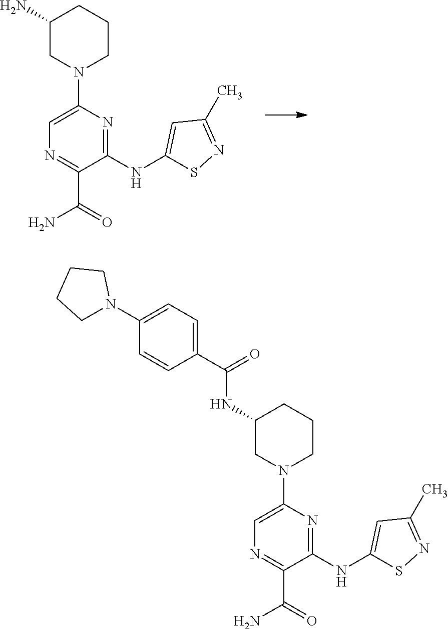 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(N5CCCC5)cc4)C3)cnc2C(N)=O)sn1]

In similar manner as described in Example A-8, 3-[(3-methyl-1,2-thiazol-5-yl)amino]-5-[(3R)-3-[4-(pyrrolidin-1-yl)benzamido]piperidin-1-yl]pyrazine-2-carboxamide (26) was prepared using 4-(1-pyrrolidinyl)benzoic acid. MS found for C25H30N8O2S as (M+H)⁺ 507.1. ¹H NMR (500 MHz, DMSO) δ 12.33 (s, 1H), 8.03 (d, J=7.83 Hz, 1H), 7.97-7.89 (m, 1H), 7.86 (s, 1H), 7.73 (d, J=8.80 Hz, 2H), 7.61-7.50 (m, 1H), 6.88 (s, 1H), 6.53 (d, J=8.80 Hz, 2H), 4.53-4.35 (m, 2H), 4.02-3.82 (m, 1H), 3.32-3.14 (m, 6H), 2.29 (s, 3H), 2.04-1.87 (m, 6H), 1.79-1.69 (m, 1H), 1.67-1.55 (m, 1H).

### Example A-27: (5-[(3R)-3-[3-(dimethylamino)benzamido]piperidin-1-yl]-3-[(3-methyl-1,2-thiazol-5-yl)amino]pyrazine-2-carboxamide hydrochloride (27)

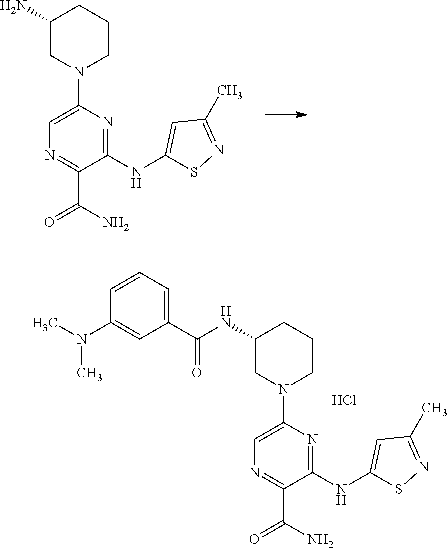

To a solution of 5-[(3R)-3-aminopiperidin-1-yl]-3-[(3-methyl-1,2-thiazol-5-yl)amino]pyrazine-2-carboxamide (150.5 mg, 0.45 mmol) in DMF (3 mL) DIPEA (0.4 mL, 2.25 mmol) and 3-(dimethylamino)benzoyl chloride hydrochloride (117.9 mg, 0.54 mmol) were added. The mixture was stirred at room temperature for 1 hour then it was purified by flash chromatography (silica) MeOH in DCM, from 0 to 5% to obtain (5-[(3R)-3-[3-(dimethylamino)benzamido]piperidin-1-yl]-3-[(3-methyl-1,2-thiazol-5-yl)amino]pyrazine-2-carboxamide. The product was dissolved in a mixture of dichloromethane and methanol and 1.25 M HCl in methanol (1 mL) was added. The mixture was stirred at room temperature for 1 hour then it was concentrated to afford (5-[(3R)-3-[3-(dimethylamino)benzamido]piperidin-1-yl]-3-[(3-methyl-1,2-thiazol-5-yl)amino]pyrazine-2-carboxamide hydrochloride (112.7 mg, yield 48%). MS found for C23H28N8O2S as (M+H)⁺ 481.4. ¹H NMR (400 MHz, DMSO) δ 12.32 (s, 1H), 8.41 (d, J=7.04 Hz, 1H), 7.91 (br. s., 1H), 7.86 (s, 1H), 7.56 (br. s., 1H), 7.43-7.20 (m, 3H), 7.10 (br. s., 1H), 6.87 (s, 1H), 4.52-4.31 (m, 2H), 4.05-3.91 (m, 1H), 3.39-3.24 (m, 2H), 2.98 (s, 6H), 2.29 (s, 3H), 2.07-1.88 (m, 2H), 1.84-1.54 (m, 2H).

### Example A-28: 5-[(3R)-3-cyclohexaneamidopiperidin-1-yl]-3-[(3-methyl-1,2-thiazol-5-yl)amino]pyrazine-2-carboxamide (28)

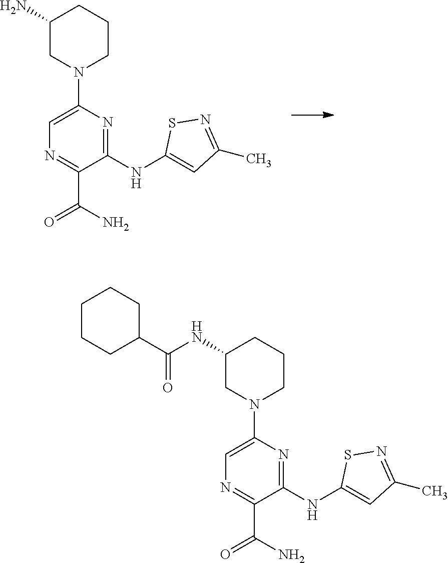 [SMILES: Cc1cc(Nc2nc(N3CCC[C@@H](N)C3)cnc2C(N)=O)sn1 ; Cc1cc(Nc2nc(N3CCC[C@@H](NC(=O)C4CCCCC4)C3)cnc2C(N)=O)sn1]

In similar manner as described in Example A-8 5-[(3R)-3-cyclohexaneamidopiperidin-1-yl]-3-[(3-methyl-1,2-thiazol-5-yl)amino]pyrazine-2-carboxamide (28) was prepared using cyclohexanecarboxylic acid. MS found for C21H29N7O2S as (M+H)⁺ 444.0. ¹H NMR (500 MHz, DMSO) δ 12.28 (s, 1H), 7.89 (br. s, 1H), 7.80-7.70 (m, 2H), 7.54 (br. s, 1H), 6.84 (s, 1H), 4.27-3.99 (m, 2H), 3.81-3.66 (m, 1H), 3.58-3.42 (m, 1H), 3.34-3.21 (m, 1H), 2.29 (s, 3H), 2.14-2.02 (m, 1H), 1.95-1.01 (m, 14H).

### Example A-29: 3-[(3-methyl-1,2-thiazol-5-yl)amino]-5-[(3R)-3-(pyridine-4-amido)piperidin-1-yl]pyrazine-2-carboxamide (29)

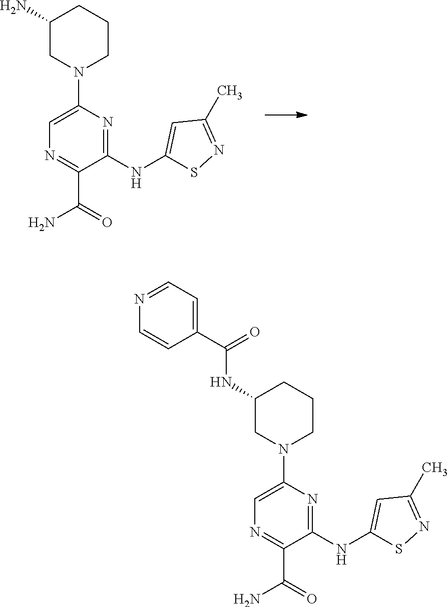 [SMILES: Cc1cc(Nc2nc(N3CCC[C@@H](N)C3)cnc2C(N)=O)sn1 ; Cc1cc(Nc2nc(N3CCC[C@@H](NC(=O)c4ccncc4)C3)cnc2C(N)=O)sn1]

In similar manner as described in Example A-8, 3-[(3-methyl-1,2-thiazol-5-yl)amino]-5-[(3R)-3-(pyridine-4-amido)piperidin-1-yl]pyrazine-2-carboxamide (29) was prepared using isonicotinic acid. MS found for C20H22N8O2S as (M+H)⁺ 439.0. ¹H NMR (500 MHz, DMSO) δ 12.29 (br. s, 1H), 8.79 (d, J=7.34 Hz, 1H), 8.77-8.73 (m, 2H), 7.92 (br. s., 1H), 7.85 (s, 1H), 7.80-7.76 (m, 2H), 7.55 (br. s., 1H), 7.55 (br. s., 1H), 6.85 (s, 1H), 4.57-4.41 (m, 1H), 4.40-4.27 (m, 1H), 4.10-3.93 (m, 1H), 3.45-3.24 (m, 2H), 2.28 (s, 3H), 2.09-1.98 (m, 1H), 1.97-1.88 (m, 1H), 1.84-1.70 (m, 1H), 1.70-1.58 (m, 1H).

### Example A-30: 3-[(3-methyl-1,2-thiazol-5-yl)amino]-5-[(3R)-3-[4-(propan-2-yl)benzamido]piperidin-1-yl]pyrazine-2-carboxamide (30)

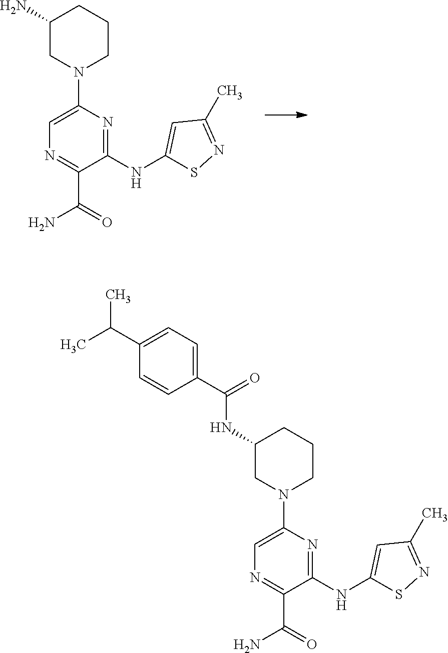 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(C(C)C)cc4)C3)cnc2C(N)=O)sn1]

In similar manner as described in Example A-8, 3-[(3-methyl-1,2-thiazol-5-yl)amino]-5-[(3R)-3-[4-(propan-2-yl)benzamido]piperidin-1-yl]pyrazine-2-carboxamide (30) was prepared using 4-isopropylbenzoic acid. MS found for C24H29N7O2S as (M+H)⁺ 480.0. ¹H NMR (500 MHz, DMSO) δ 12.29 (s, 1H), 8.36 (d, J=7.34 Hz, 1H), 7.91 (br. s., 1H), 7.84 (s, 1H), 7.76 (d, J=8.31 Hz, 2H), 7.54 (br. s., 1H), 7.33 (d, J=8.31 Hz, 2H), 6.85 (s, 1H), 4.55-4.31 (m, 2H), 4.05-3.89 (m, 1H), 3.35-3.24 (m, 2H), 2.94 (spt, J=6.93 Hz, 1H), 2.28 (s, 3H), 2.05-1.87 (m, 2H), 1.79-1.56 (m, 2H), 1.22 (d, J=6.85 Hz, 6H).

### Example A-31: N-[(3R)-1-{5-carbamoyl-6-[(3-methyl-1,2-thiazol-5-yl)amino]pyrazin-2-yl}piperidin-3-yl]-1-ethyl-1H-indole-5-carboxamide (31)

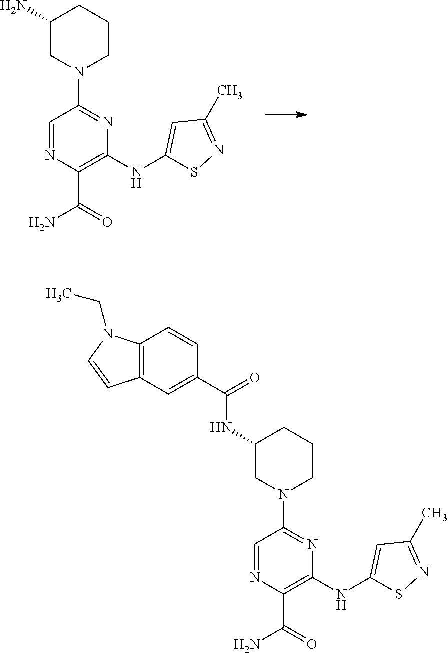 [SMILES: Cc1cc(Nc2nc(N3CCC[C@@H](N)C3)cnc2C(N)=O)sn1 ; CCn1ccc2cc(C(=O)N[C@@H]3CCCN(c4cnc(C(N)=O)c(Nc5cc(C)ns5)n4)C3)ccc21]

In similar manner as described in Example A-8, N-[(3R)-1-{5-carbamoyl-6-[(3-methyl-1,2-thiazol-5-yl)amino]pyrazin-2-yl}piperidin-3-yl]-1-ethyl-1H-indole-5-carboxamide (31) was prepared using 1-ethyl-1H-indole-5-carboxylic acid. MS found for C25H28N8O2S as (M+H)⁺ 505.0. ¹H NMR (500 MHz, DMSO) δ 12.30 (s, 1H), 8.31 (d, J=7.83 Hz, 1H), 8.12 (s, 1H), 7.91 (br. s., 1H), 7.86 (s, 1H), 7.68 (dd, J=8.80, 1.47 Hz, 1H), 7.58-7.50 (m, 2H), 7.48 (d, J=2.93 Hz, 1H), 6.84 (s, 1H), 6.54 (d, J=3.42 Hz, 1H), 4.59-4.34 (m, 2H), 4.24 (q, J=7.01 Hz, 2H), 4.03-3.96 (m, 1H), 3.31-3.24 (m, 2H), 2.27 (s, 3H), 2.06-2.00 (m, 1H), 1.97-1.90 (m, 1H), 1.84-1.71 (m, 1H), 1.70-1.59 (m, 1H), 1.36 (t, J=7.34 Hz, 3H).

### Example A-32: 5-[(3R)-3-(4-cycldopropylbenzamido)piperidin-1-yl]-3-[(3-methyl-1,2-thiazol-5-yl)amino]pyrazine-2-carboxamide (32)

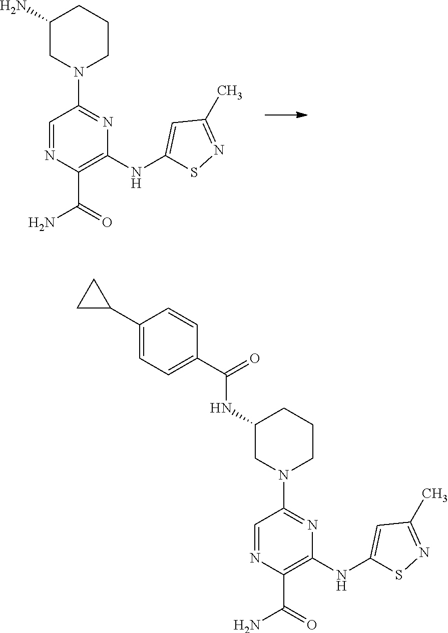 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(C5CC5)cc4)C3)cnc2C(N)=O)sn1]

In similar manner as described in Example A-8, 5-[(3R)-3-(4-cyclopropylbenzamido)piperidin-1-yl]-3-[(3-methyl-1,2-thiazol-5-yl)amino]pyrazine-2-carboxamide (32) was prepared using 4-cyclopropylbenzoic acid. MS found for C24H27N7O2S as (M+H)⁺ 478.0. ¹H NMR (500 MHz, DMSO) δ 12.29 (s, 1H), 8.34 (d, J=7.83 Hz, 1H), 7.91 (br. s., 1H), 7.84 (s, 1H), 7.73 (d, J=8.31 Hz, 2H), 7.54 (br. s., 1H), 7.15 (d, J=8.31 Hz, 2H), 6.84 (s, 1H), 4.43 (br. s., 2H), 4.00-3.87 (m, 1H), 3.31-3.20 (m, 2H), 2.27 (s, 3H), 2.05-1.57 (m, 5H), 1.04-0.97 (m, 2H), 0.76-0.70 (m, 2H).

### Example A-33: 3-[(3-methyl-1,2-thiazol-5-yl)amino]-5-[(3R)-3-(3-methylbenzamido)piperidin-1-yl]pyrazine-2-carboxamide (33)

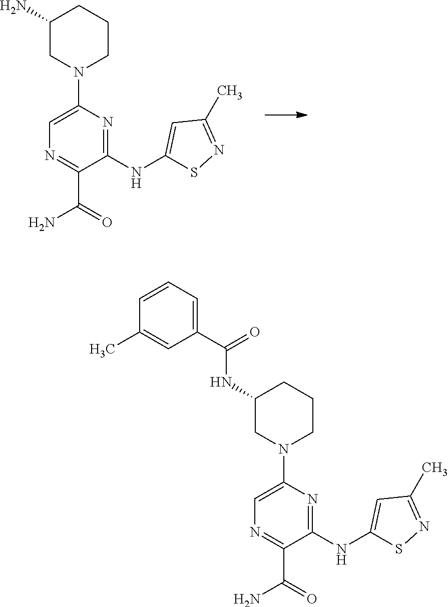

In similar manner as described in Example A-8, 3-[(3-methyl-1,2-thiazol-5-yl)amino]-5-[(3R)-3-(3-methylbenzamido)piperidin-1-yl]pyrazine-2-carboxamide (33) was prepared using 3-methylbenzoic acid. MS found for C22H25N7O2S as (M+H)⁺ 452.0. ¹H NMR (500 MHz, DMSO) δ 12.29 (s, 1H), 8.40 (d, J=7.41 Hz, 1H), 7.91 (br. s., 1H), 7.84 (s, 1H), 7.62 (m, J=2.50 Hz, 2H), 7.55 (br. s, 1H), 7.37-7.31 (m, 2H), 6.85 (s, 1H), 4.57-4.23 (m, 2H), 4.01-3.92 (m, 1H), 3.42-3.26 (m, 2H), 2.35 (s, 3H), 2.28 (s, 3H), 2.06-1.97 (m, 1H), 1.97-1.89 (m, 1H), 1.81-1.70 (m, 1H), 1.69-1.58 (m, 1H).

### Example A-34: 5-[(3R)-3-[6-(dimethylamino)pyridine-3-amido]piperidin-1-yl]-3-[(3-methyl-1,2-thiazol-5-yl)amino]pyrazine-2-carboxamide (34)

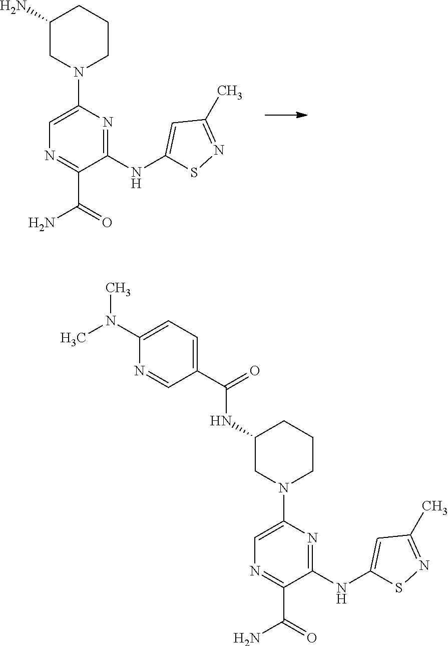

In similar manner as described in Example A-8, 5-[(3R)-3-[6-(dimethylamino)pyridine-3-amido]piperidin-1-yl]-3-[(3-methyl-1,2-thiazol-5-yl)amino]pyrazine-2-carboxamide (34) was prepared using 6-(dimethylamino)pyridine-3-carboxylic acid. MS found for C22H27N9O2S as (M+H)⁺ 482.0. ¹H NMR (500 MHz, DMSO) δ 12.26 (s, 1H), 8.59 (d, J=1.96 Hz, 1H), 8.19-8.14 (m, 1H), 7.99-7.89 (m, 2H), 7.84 (s, 1H), 7.58-7.49 (m, 1H), 6.84 (s, 1H), 6.65 (d, J=8.80 Hz, 1H), 4.57-4.24 (m, 2H), 4.06-3.76 (m, 1H), 3.31-3.17 (m, 2H), 3.08 (s, 6H), 2.28 (s, 3H), 2.05-1.96 (m, 1H), 1.96-1.88 (m, 1H), 1.80-1.68 (m, 1H), 1.67-1.54 (m, 1H).

### Example A-35: 3-[(3-methyl-1,2-thiazol-5-yl)amino]-5-[(3R)-3-[4-(piperidin-1-yl)benzamido]piperidin-1-yl]pyrazine-2-carboxamide (35)

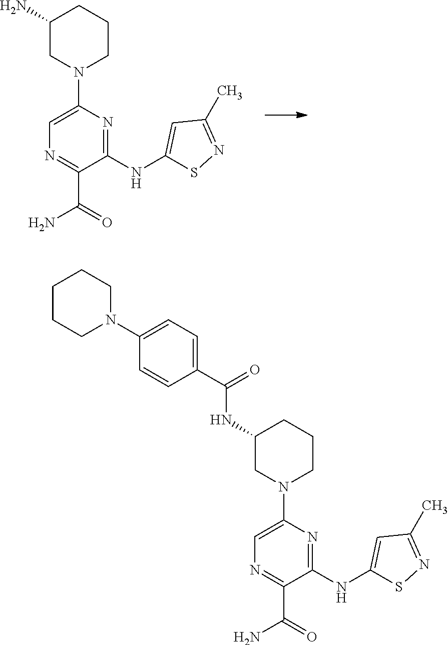 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(N5CCCCC5)cc4)C3)cnc2C(N)=O)sn1]

In similar manner as described in Example A-8 3-[(3-methyl-1,2-thiazol-5-yl)amino]-5-[(3R)-3-[4-(piperidin-1-yl)benzamido]piperidin-1-yl]pyrazine-2-carboxamide (35) was prepared using 4-(piperidin-1-yl)benzoic acid. MS found for C26H32N8O2S as (M+H)⁺ 521.0. ¹H NMR (500 MHz, DMSO) δ 12.29 (s, 1H), 8.13 (d, J=7.34 Hz, 1H), 7.91 (br. s., 1H), 7.84 (s, 1H), 7.73 (d, J=8.80 Hz, 2H), 7.54 (br. s., 1H), 6.95 (d, J=8.31 Hz, 2H), 6.85 (s, 1H), 4.45 (br. s., 2H), 3.99-3.89 (m, 1H), 3.31-3.18 (m, 6H), 2.28 (s, 3H), 2.04-1.53 (m, 10H).

### Example A-36: 5-[(3R)-3-(2,4-difluorobenzamido)piperidin-1-yl]-3-[(3-methyl-1,2-thiazol-5-yl)amino]pyrazine-2-carboxamide (36)

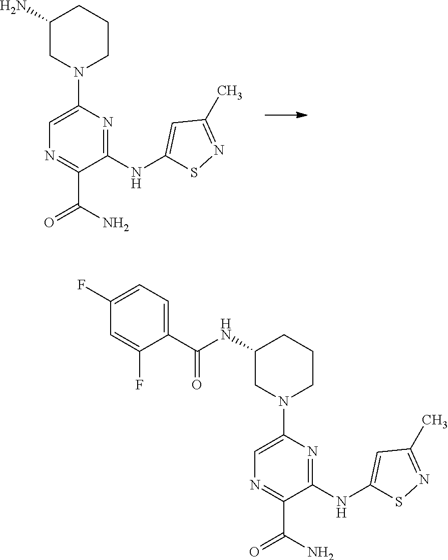 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(F)cc4F)C3)cnc2C(N)=O)sn1]

In similar manner as described in Example A-7 5-[(3R)-3-(2,4-difluorobenzamido)piperidin-1-yl]-3-[(3-methyl-1,2-thiazol-5-yl)amino]pyrazine-2-carboxamide (36) was prepared using 2,4-difluorobenzoyl chloride. MS found for C21H21F2N7O2S as (M+H)⁺ 473.9. ¹H NMR (500 MHz, DMSO) δ 12.29 (s, 1H), 8.45 (d, J=7.34 Hz, 1H), 7.91 (br. s., 1H), 7.82 (s, 1H), 7.61 (td, J=8.31, 6.85 Hz, 1H), 7.54 (br. s., 1H), 7.34 (td, J=9.90, 2.20 Hz, 1H), 7.16 (td, J=8.44, 2.20 Hz, 1H), 6.85 (s, 1H), 4.43-4.26 (m, 1H), 4.25-4.12 (m, 1H), 4.06-3.91 (m, 1H), 3.57-3.42 (m, 2H), 2.29 (s, 3H), 1.99 (s, 2H), 1.78-1.56 (m, 2H).

### Example A-37: 5-[(3R)-3-(4-tert-butylbenzamido)piperidin-1-yl]-3-[(3-methyl-1,2-thiazol-5-yl)amino]pyrazine-2-carboxamide (37)

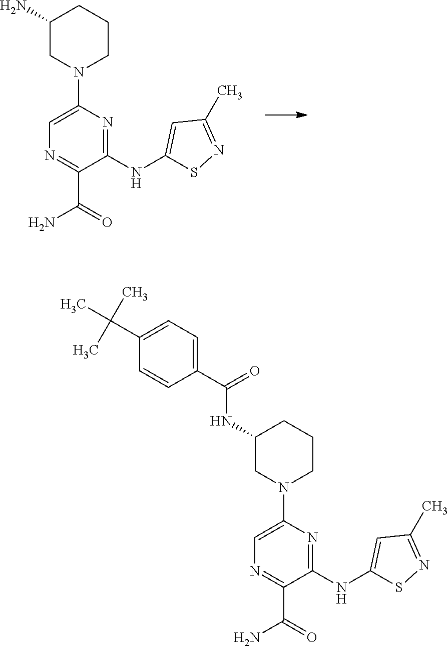 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(C(C)(C)C)cc4)C3)cnc2C(N)=O)sn1]

In similar manner as described in Example A-8 5-[(3R)-3-(4-tert-butylbenzamido)piperidin-1-yl]-3-[(3-methyl-1,2-thiazol-5-yl)amino]pyrazine-2-carboxamide (37) was prepared using 4-tert-butylbenzoic acid. MS found for C25H31N7O2S as (M+H) 494.0. ¹H NMR (500 MHz, DMSO) δ 12.24 (s, 1H), 12.24 (s, 1H), 8.37 (d, J=7.41 Hz, 1H), 7.91 (br. s., 1H), 7.84 (s, 1H), 7.76 (d, J=8.23 Hz, 2H), 7.54 (br. s., 1H), 7.47 (d, J=8.51 Hz, 2H), 6.84 (s, 1H), 4.55-4.30 (m, 2H), 4.00-3.91 (m, 1H), 3.36-3.32 (m, 1H), 3.30-3.25 (m, 1H), 2.28 (s, 3H), 1.99 (s, 2H), 1.81-1.57 (m, 2H), 1.30 (s, 9H).

### Example A-38: N-[(3R)-1-{5-carbamoyl-6-[(3-methyl-1,2-thiazol-5-yl)amino]pyrazin-2-yl}piperidin-3-yl]-2-ethyl-2H-indazole-5-carboxamide (38)

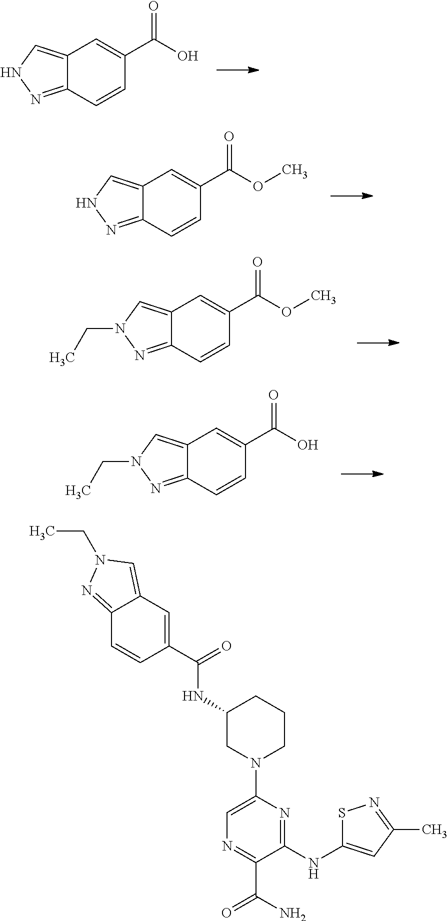

To a suspension of 1H-indazole-5-carboxylic acid (470 mg, 2.9 mmol) in MeOH (5 mL) H₂SO₄ (0.2 mL) was added. The mixture was heated to 70° C. and stirred at this temperature overnight. The mixture was left to reach room temperature, H₂O (10 mL), NaHCO₃ saturated aqueous solution (5 mL) and ethyl acetate (30 mL) were added. The phases were separated, the aqueous phase was extracted with ethyl acetate. The combined organic phases were dried over Na₂SO₄ and evaporated to dryness to give methyl 1H-indazole-5-carboxylate (466 mg, 2.65 mmol, 88% yield) as a pale pink-yellow solid. MS found for C9H8N2O2 as (M+H)⁺ 176.9.

To a solution of 1H-indazole-5-carboxylate (466 mg, 2.65 mmol) in DMF (10 mL) K₂CO₃ (721 mg, 5.22 mmol) and CH₃CH₂I (0.25 mL, 3.13 mmol) were added. The mixture was stirred at room temperature overnight then it was filtered. The solution was concentrated and submitted to preparative purification to give methyl 2-ethyl-2H-indazole-5-carboxylate (121 mg, 0.59 mmol, 23% yield). MS found for C11H12N2O2 as (M+H)⁺ 205.9. To a solution of methyl 2-ethyl-2H-indazole-5-carboxylate (121 mg, 0.59 mmol) in MeOH (3 mL) a solution of NaOH (95.3 mg, 2.37 mmol) in H₂O (0.5 mL) was added. The mixture was stirred at room temperature for 20 hours. H₂O and DCM were added, the phases were separated and the organic one dried over Na₂SO₄ and evaporated to dryness to give 2-ethyl-2H-indazole-5-carboxylic acid (101.3 mg, 90% yield). MS found for C10H10N2O2 as (M+H)⁺ 190.9.

In similar manner as described in Example A-8 N-[(3R)-1-{5-carbamoyl-6-[(3-methyl-1,2-thiazol-5-yl)amino]pyrazin-2-yl}piperidin-3-yl]-2-ethyl-2H-indazole-5-carboxamide (38) was prepared using 2-ethyl-2H-indazole-5-carboxylic acid. MS found for C24H27N9O2S as (M+H)⁺ 506.3. ¹H NMR (400 MHz, DMSO) δ 12.27 (s, 1H), 8.55 (s, 1H), 8.39 (d, J=7.45 Hz, 1H), 8.27 (s, 1H), 7.89 (br. s., 1H), 7.84 (s, 1H), 7.66-7.71 (m, 1H), 7.58-7.63 (m, 1H), 7.52 (br. s., 1H), 6.83 (s, 1H), 4.34-4.54 (m, 4H), 3.89-4.05 (m, 1H), 3.23-3.34 (m, 2H), 2.25 (s, 3H), 1.87-2.07 (m, 2H), 1.56-1.82 (m, 2H), 1.50 (t, J=7.24 Hz, 3H).

### Example A-39: 5-[(3R)-3-(4-cyclopropyl-2-fluorobenzamido)piperidin-1-yl]-3-[(3-methyl-1,2-thiazol-5-yl)amino]pyrazine-2-carboxamide (39)

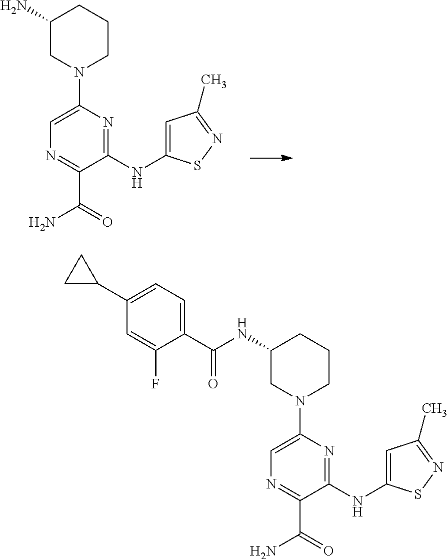

In similar manner as described in Example A-8, 5-[(3R)-3-(4-cyclopropyl-2-fluorobenzamido)piperidin-1-yl]-3-[(3-methyl-1,2-thiazol-5-yl)amino]pyrazine-2-carboxamide (39) was prepared using 4-cyclopropyl-2-fluorobenzoic acid. MS found for C24H26FN7O2S as (M+H)⁺ 496.3. ¹H NMR (400 MHz, Chloroform) δ 11.82 (s, 1H), 7.96 (t, J=8.33 Hz, 1H), 7.68 (s, 1H), 7.47-7.36 (m, 1H), 6.95 (dd, J=8.11, 1.32 Hz, 1H), 6.74 (d, J=1.32 Hz, 2H), 6.64 (s, 1H), 5.29 (br. s., 1H), 4.38-4.07 (m, 3H), 3.86-3.72 (m, 1H), 3.69-3.60 (m, 1H), 2.41 (s, 3H), 2.16-1.70 (m, 5H), 1.17-1.01 (m, 2H), 0.81-0.69 (m, 2H).

### Example A-40: 3-[(3-methyl-1,2-thiazol-5-yl)amino]-5-[(3R)-3-[N-methyl4-(propan-2-yl)benzamido]piperidin-1-yl]pyrazine-2-carboxamide (Compound 40)

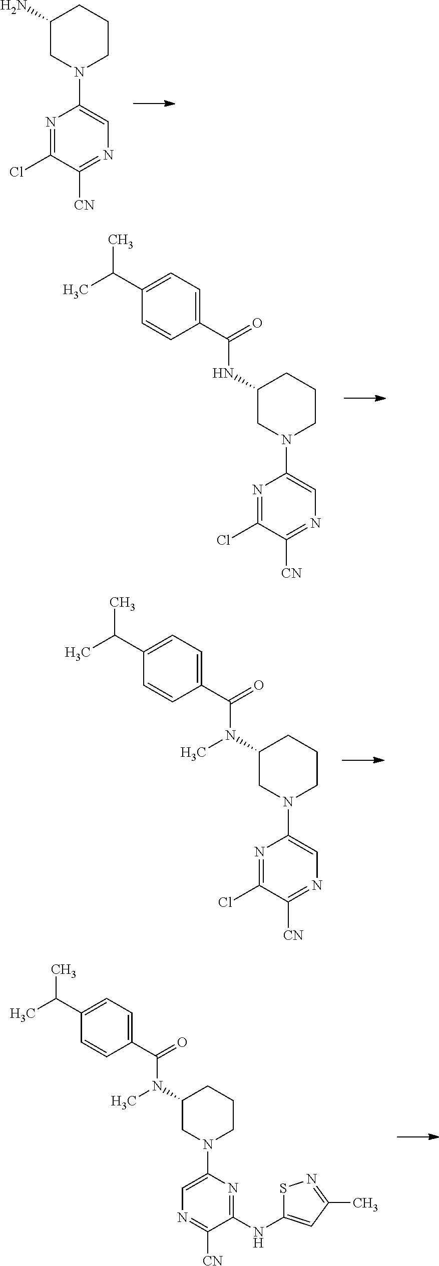 [SMILES: N#Cc1ncc(N2CCC[C@@H](N)C2)nc1Cl ; CC(C)c1ccc(C(=O)N[C@@H]2CCCN(c3cnc(C#N)c(Cl)n3)C2)cc1 ; CC(C)c1ccc(C(=O)N(C)[C@@H]2CCCN(c3cnc(C#N)c(Cl)n3)C2)cc1 ; Cc1cc(Nc2nc(N3CCC[C@@H](N(C)C(=O)c4ccc(C(C)C)cc4)C3)cnc2C#N)sn1]

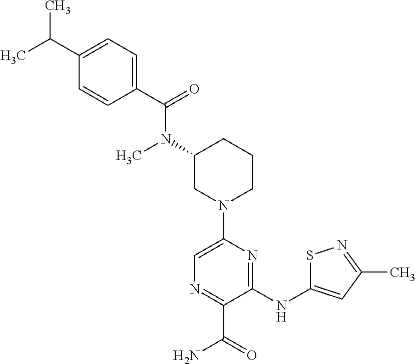 [SMILES: Cc1cc(Nc2nc(N3CCC[C@@H](N(C)C(=O)c4ccc(C(C)C)cc4)C3)cnc2C(N)=O)sn1]

In similar manner as described in Example A-8, N-[(3R)-1-(6-chloro-5-cyanopyrazin-2-yl)piperidin-3-yl]-4-(propan-2-yl)benzamide was prepared using 4-(propan-2-yl)benzoic acid. MS found for C20H22ClN5O as (M+H)⁺ 384.3.

To a solution of N-[(3R)-1-(6-chloro-5-cyanopyrazin-2-yl)piperidin-3-yl]-4-(propan-2-yl)benzamide (300 mg, 0.78 mmol) in THF (7 mL) NaH 60% dispersion in mineral oil (53.5 mg, 1.33 mmol) and CH₃I (75 μL, 1.18 mmol) were added. The mixture was stirred at 60° C. for 5 hours. Further NaH 60% dispersion in mineral oil (53 mg, 1.33 mmol) and CH₃I (75 μL, 1.18 mmol) were added and the mixture was stirred at 60° C. for 2 hours. The solution was left to reach room temperature then it was diluted with ethyl acetate (50 mL) and washed with H₂O (30 mL). The organic phase was dried over Na₂SO₄ and concentrated. The obtained crude was purified by flash chromatography (silica), ethyl acetate in cyclohexane from 50 to 100% to give N-[(3R)-1-(6-chloro-5-cyanopyrazin-2-yl)piperidin-3-yl]-N-methyl-4-(propan-2-yl)benzamide (162 mg, 53% yield) as a white foam.

In similar manner as described in Example A-1, N-[(3R)-1-{5-cyano-6-[(3-methyl-1,2-thiazol-5-yl)amino]pyrazin-2-yl}piperidin-3-yl]-N-methyl-4-(propan-2-yl)benzamide was prepared using 3-methyl-1,2-thiazol-5-amine hydrochloride. MS found for C25H29N7OS as (M+H)⁺ 476.0.

To a solution of N-[(3R)-1-{5-cyano-6-[(3-methyl-1,2-thiazol-5-yl)amino]pyrazin-2-yl}piperidin-3-yl]-N-methyl-4-(propan-2-yl)benzamide (84 mg, 0.18 mmol) in MeOH/DMSO (2/0.2 mL), NaOH (22 mg, 0.55 mmol) and H₂O₂ 30% in water (0.1 mL) were added. The mixture was stirred at room temperature for 2 hours then it was partitioned between ethyl acetate and water. The organic phase was dried over Na₂SO₄, concentrated and purified by flash chromatography silica, MeOH in DCM from 0 to 3% to give 3-[(3-methyl-1,2-thiazol-5-yl)amino]-5-[(3R)-3-[N-methyl4-(propan-2-yl)benzamido]piperidin-1-yl]pyrazine-2-carboxamide (47.3 mg, 53% yield) as a yellow solid. MS found for C25H31 N7O2S as (M+H)⁺ 494.4. ¹H NMR (400 MHz, DMSO) δ 12.24 (s, 1H), 7.95-7.73 (m, 2H), 7.57 (br. s., 1H), 7.45-7.06 (m, 4H), 6.87 (s, 1H), 4.91-4.22 (m, 3H), 3.47-2.77 (m, 6H), 2.31 (s, 3H), 2.11-1.52 (m, 4H), 1.36-1.03 (m, 6H).

### Example A-42: Synthesis of 3-[(4-methanesulfonylphenyl)amino]-5-[(3R)-3-[(phenylcarbamoyl)amino]piperidin-1-yl]pyrazine-2-carboxamide (42)

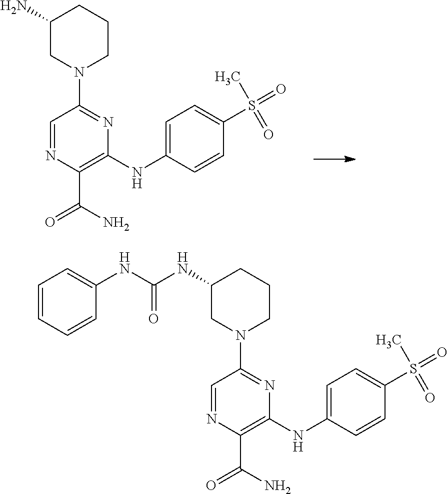 [SMILES: CS(=O)(=O)c1ccc(Nc2nc(N3CCC[C@@H](N)C3)cnc2C(N)=O)cc1 ; CS(=O)(=O)c1ccc(Nc2nc(N3CCC[C@@H](NC(=O)Nc4ccccc4)C3)cnc2C(N)=O)cc1]

In a similar manner as described in Example A-1, 3-[(4-methanesulfonylphenyl)amino]-5-[(3R)-3-[(phenylcarbamoyl)amino]piperidin-1-yl]pyrazine-2-carboxamide (42) was prepared using isocyanatobenzene. MS found for C24H27N7O4S as (M+H)⁺ 510.0. ¹H NMR (400 MHz, DMSO) δ 11.88 (s, 1H), 8.38 (s, 1H), 7.95-7.75 (m, 6H), 7.48 (d, J=2.26 Hz, 1H), 7.40 (dd, J=7.90, 1.00 Hz, 2H), 7.23 (t, J=7.90 Hz, 2H), 6.89 (t, J=7.90 Hz, 1H), 6.31 (d, J=7.03 Hz, 1H), 4.28 (d, J=10.79 Hz, 1H), 3.99-3.85 (m, 1H), 3.80-3.68 (m, 1H), 3.58-3.45 (m, 1H), 3.39-3.26 (m, 1H), 3.08 (s, 3H), 2.04-1.89 (m, 1H), 1.86-1.73 (m, 1H), 1.72-1.49 (m, 2H).

### Example A-43: Synthesis of 3-[(4-methanesulfonylphenyl)amino]-5-[(3R)-3-{[(pyridin-3-yl)carbamoyl]amino}piperidin-1-yl]pyrazine-2-carboxamide (43)

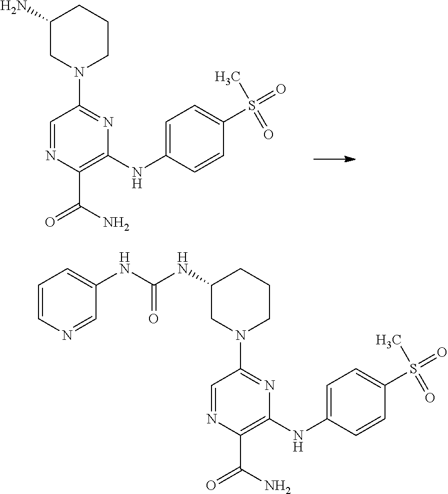 [SMILES: CS(=O)(=O)c1ccc(Nc2nc(N3CCC[C@@H](N)C3)cnc2C(N)=O)cc1 ; CS(=O)(=O)c1ccc(Nc2nc(N3CCC[C@@H](NC(=O)Nc4cccnc4)C3)cnc2C(N)=O)cc1]

In a similar manner as described in Example A-1, 3-[(4-methanesulfonylphenyl)amino]-5-[(3R)-3-{[(pyridin-3-yl)carbamoyl]amino}piperidin-1-yl]pyrazine-2-carboxamide (43) was prepared using 3-isocyanatopyridine. MS found for C23H26N8O4S as (M+H)⁺ 511.0. ¹H NMR (400 MHz, DMSO) δ 11.87 (s, 1H) 8.59 (s, 1H), 8.45 (d, J=2.51 Hz, 1H), 8.11 (dd, J=4.77, 1.51 Hz, 1H), 8.01-7.94 (m, 1H), 7.91-7.79 (m, 6H), 7.49 (d, J=2.26 Hz, 1H), 7.26 (dd, J=8.28, 4.70 Hz, 1H), 6.50 (d, J=7.28 Hz, 1H), 4.32-4.16 (m, 1H), 3.97-3.84 (m, 1H), 3.83-3.70 (m, 1H), 3.62-3.46 (m, 1H), 3.39 (dd, J=12.80, 7.78 Hz, 1H), 3.10 (s, 3H), 2.05-1.89 (m, 1H), 1.87-1.74 (m, 1H), 1.72-1.54 (m, 2H).

### Example A-44: Synthesis of 5-[(3R)-3-[4-(dimethylamino)benzamido]piperidin-1-yl]-3-[(4-methanesulfonylphenyl)amino]pyrazine-2-carboxamide (44)

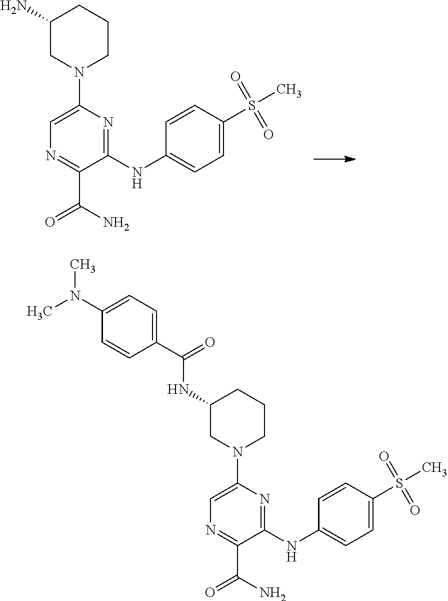

In a similar manner as described in Example A-7, 5-[(3R)-3-[4-(dimethylamino)benzamido]piperidin-1-yl]-3-[(4-methanesulfonylphenyl)amino]pyrazine-2-carboxamide (44) was prepared using 4-(dimethylamino)benzoyl chloride. MS found for C26H31N7O4S as (M+H)⁺ 538.1. ¹H NMR (400 MHz, DMSO) δ 11.81 (s, 1H), 8.03 (d, J=7.68 Hz, 1H), 7.90-7.83 (m, 5H), 7.82 (s, 1H), 7.79 (d, J=8.78 Hz, 2H), 7.48 (br. s., 1H), 6.69 (d, J=8.78 Hz, 2H), 4.61-4.44 (m, 1H), 4.23 (d, J=12.63 Hz, 1H), 4.01-3.87 (m, 1H), 3.19-3.12 (m, 1H), 3.11 (s, 3H), 3.04-2.98 (m, 1H), 2.97 (s, 6H), 2.02-1.85 (m, 2H), 1.82-1.71 (m, 1H), 1.67-1.54 (m, 1H).

### Example A-45: Synthesis of 5-[(3R)-3-benzamidopiperidin-1-yl]-3-[(quinolin-6-yl)amino]pyrazine-2-carboxamide (45)

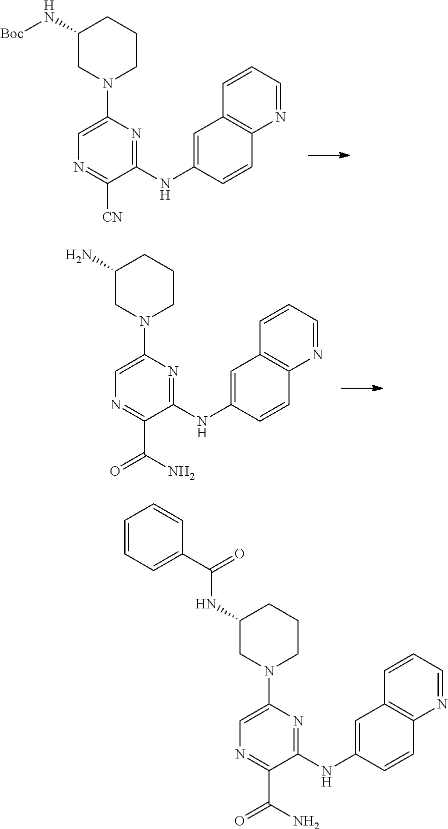

In a similar manner as described in Example A-I, tert-butyl N-[(3R)-1-{5-cyano-6-[(quinolin-6-yl)amino]pyrazin-2-yl}piperidin-3-yl]carbamate was prepared using 6-aminoquinoline. MS found for C24H27N7O2 as (M+H)⁺ 446.0.

Tert-butyl N-[(3R)-1-{5-cyano-6-[(quinolin-6-yl)amino]pyrazin-2-yl}piperidin-3-yl]carbamate (1.0 g, 2.24 mmol), was dissolved in TFA (10 ml) and H₂SO₄ (2 ml) was added. The mixture was refluxed for 30 min then the solvent was removed. The resulting oil was treated with K₂CO₃ aqueous saturated solution. The solvent was removed and the residue washed with MeOH several times. The solution was then loaded onto a SCX cartridge. 5-[(3R)-3-aminopiperidin-1-yl]-3-[(quinolin-6-yl)amino]pyrazine-2-carboxamide was obtained with NH₃ in methanol (520.0 mg, 64% yield).

In a similar manner as described in Example A-7, 5-[(3R)-3-benzamidopiperidin-1-yl]-3-[(quinolin-6-yl)amino]pyrazine-2-carboxamide (45) was prepared using benzoyl chloride. MS found for C26H25N7O2 as (M+H)⁺ 468.0. ¹H NMR (400 MHz, DMSO) δ 11.92 (s, 1H), 8.84 (d, J=3.76 Hz, 1H), 8.67-8.39 (m, 3H), 8.05 (d, J=9.29 Hz, 1H), 7.98-7.88 (m, 4H), 7.85 (s, 1H), 7.64-7.47 (m, 4H), 7.39 (br. s., 1H), 4.62 (d, J=10.04 Hz, 1H), 4.25 (d, J=13.30 Hz, 1H), 4.05 (br. s., 1H), 3.31-3.02 (m, 2H), 2.11-1.90 (m, 2H), 1.86-1.57 (m, 2H).

### Example A-46: Synthesis of 5-[(3R)-3-[4-(dimethylamino)benzamido]piperidin-1-yl]-3-[(quinolin-6-yl)amino]pyrazine-2-carboxamide hydrochloride (46)

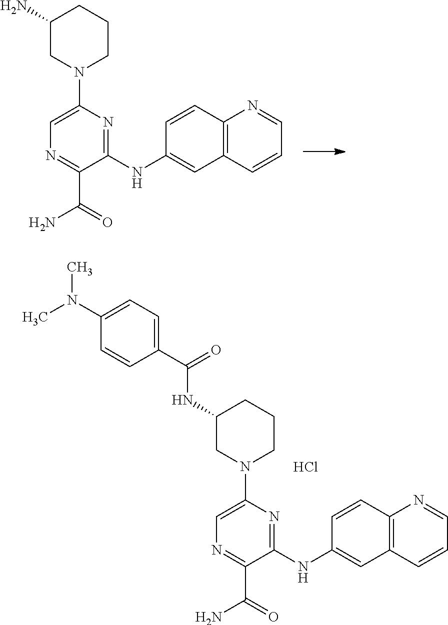 [SMILES: NC(=O)c1ncc(N2CCC[C@@H](N)C2)nc1Nc1ccc2ncccc2c1 ; CN(C)c1ccc(C(=O)N[C@@H]2CCCN(c3cnc(C(N)=O)c(Nc4ccc5ncccc5c4)n3)C2)cc1 ; Cl]

In a similar manner as described in Example A-7, 5-[(3R)-3-[4-(dimethylamino)benzamido]piperidin-1-yl]-3-[(quinolin-6-yl)amino]pyrazine-2-carboxamide hydrochloride (46) was prepared using 4-(dimethylamino)benzoyl chloride. MS found for C28H30N8O2 as (M+H)⁺ 511.0. ¹H NMR (400 MHz, DMSO) δ 11.82-11.67 (m, 1H), 8.67 (dd, J=4.14, 1.63 Hz, 1H), 8.45 (d, J=2.51 Hz, 1H), 8.19-8.10 (m, 2H), 7.95-7.76 (m, 5H), 7.70 (dd, J=9.03, 2.26 Hz, 1H), 7.45 (br. s, 1H), 7.23-7.11 (m, 1H), 6.76 (d, J=9.03 Hz, 2H), 4.79-4.57 (m, 1H), 4.28 (d, J=13.05 Hz, 1H), 4.14-3.94 (m, 1H), 3.19-3.08 (m, 1H), 3.07-3.02 (m, 1H), 3.00 (s, 6H), 2.08-1.84 (m, 2H), 1.82-1.58 (m, 2H).

### Example A-47: Synthesis of 5-[(3R)-3-[(dimethylcarbamoyl)amino]piperidin-1-yl]-3-[(quinolin-6-yl)amino]pyrazine-2-carboxamide (47)

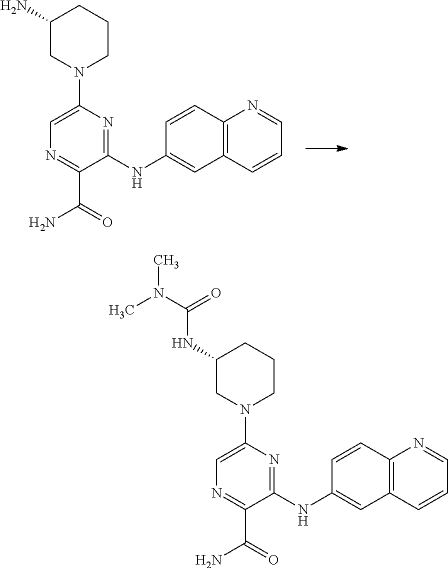 [SMILES: NC(=O)c1ncc(N2CCC[C@@H](N)C2)nc1Nc1ccc2ncccc2c1 ; CN(C)C(=O)N[C@@H]1CCCN(c2cnc(C(N)=O)c(Nc3ccc4ncccc4c3)n2)C1]

In a similar manner as described in Example A-7, 5-[(3R)-3-[4-(dimethylamino)benzamido]piperidin-1-yl]-3-[(quinolin-6-yl)amino]pyrazine-2-carboxamide (47) was prepared using dimethylcarbamyl chloride. MS found for C22H26N8O2 as (M+H)⁺ 435.0. ¹H NMR (400 MHz, DMSO) δ 11.74 (s, 1H), 8.73 (dd, J=4.14, 1.63 Hz, 1H), 8.45 (d, J=2.26 Hz, 1H), 8.27-8.20 (m, 1H), 7.94 (d, J=9.03 Hz, 1H), 7.85 (d, J=1.76 Hz, 1H), 7.77 (s, 1H), 7.73 (dd, J=9.03, 2.51 Hz, 1H), 7.47-7.38 (m, 2H), 6.21 (d, J=7.53 Hz, 1H), 4.67-4.19 (m, 2H), 3.80-3.58 (m, 1H), 3.06 (d, J=11.29 Hz, 1H), 2.92 (dd, J=12.55, 10.29 Hz, 1H), 2.84 (s, 6H), 2.01-1.73 (m, 2H), 1.71-1.50 (m, 2H).

### Example A-48: Synthesis of 5-[(3R)-3-benzamidopiperidin-1-yl]-3-{[4-(1-cyclopropyl-4-methylpiperidin-4-yl)phenyl]amino}pyrazine-2-carboxamide (48)

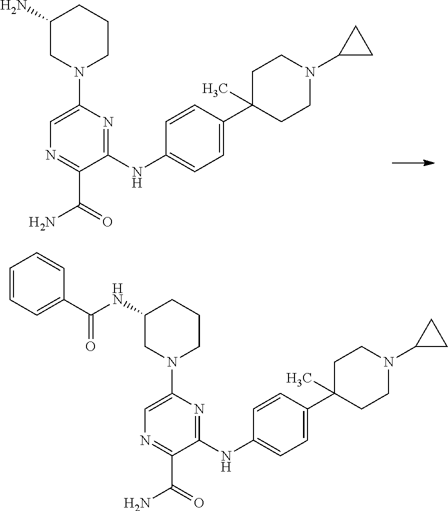

In a similar manner as described in Example A-45, 5-[(3R)-3-benzamidopiperidin-1-yl]-3-{[4-(1-cyclopropyl-4-methylpiperidin-4-yl)phenyl]amino}pyrazine-2-carboxamide (48) was prepared using benzoyl chloride. MS found for C32H39N7O2 as (M+H)⁺ 554.3. ¹H NMR (400 MHz, DMSO) δ 11.20 (s, 1H), 8.45 (d, J=7.68 Hz, 1H), 7.91 (d, J=7.14 Hz, 2H), 7.76 (br. s., 1H), 7.70 (s, 1H), 7.58-7.52 (m, 3H), 7.51-7.44 (m, 2H), 7.32 (br. s., 1H), 7.19 (d, J=8.78 Hz, 2H), 4.56 (d, J=12.08 Hz, 1H), 4.21 (d, J=13.17 Hz, 1H), 4.08-3.92 (m, 1H), 3.17-3.07 (m, 1H), 3.00-2.90 (m, 1H), 2.54-2.35 (m, 4H), 2.05-1.43 (m, 9H), 1.05 (s, 3H), 0.41-0.31 (m, 2H), 0.27-0.19 (m, 2H).

### Example A-49: Synthesis of 3-{[4-(1-cyclopropyl-4-methylpiperidin-4-yl)phenyl]amino}-5-[(3R)-3-[4-(dimethylamino)benzamido]piperidin-1-yl]pyrazine-2-carboxamide (49)

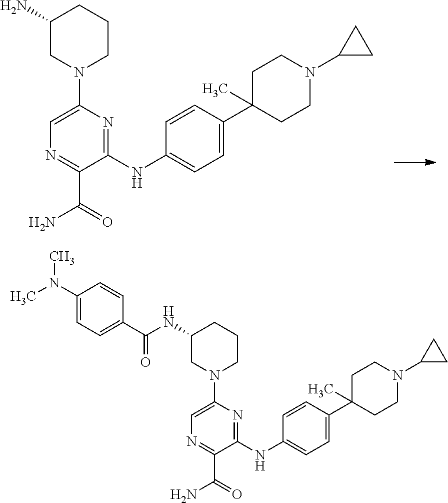

In a similar manner as described in Example A-45, 3-{[4-(1-cyclopropyl-4-methylpiperidin-4-yl)phenyl]amino}-5-[(3R)-3-[4-(dimethylamino)benzamido]piperidin-1-yl]pyrazine-2-carboxamide (49) was prepared using 4-(dimethylamino)benzoyl chloride. MS found for C34H44N8O2 as (M+H)⁺ 597.3. ¹H NMR (400 MHz, DMSO) δ 11.17 (s, 1H), 8.06 (d, J=7.69 Hz, 1H), 7.80 (d, J=8.78 Hz, 2H), 7.75 (br. s., 1H), 7.69 (s, 1H), 7.52 (d, J=8.78 Hz, 2H), 7.31 (br. s., 1H), 7.18 (d, J=8.78 Hz, 2H), 6.71 (d, J=8.78 Hz, 2H), 4.62-4.49 (m, 1H), 4.23 (d, J=13.17 Hz, 1H), 4.05-3.90 (m, 1H), 3.12-3.04 (m, 1H), 2.99 (s, 6H), 2.94-2.85 (m, 1H), 2.61-2.32 (m, 4H), 2.03-1.40 (m, 9H), 1.05 (s, 3H), 0.40-0.31 (m, 2H), 0.29-0.18 (m, 2H).

### Example A-50: Synthesis 5-[(3R)-3-benzamidopyrrolidin-1-yl]-3-[(3-methyl-1,2-thiazol-5-yl)amino]pyrazine-2-carboxamide (50)

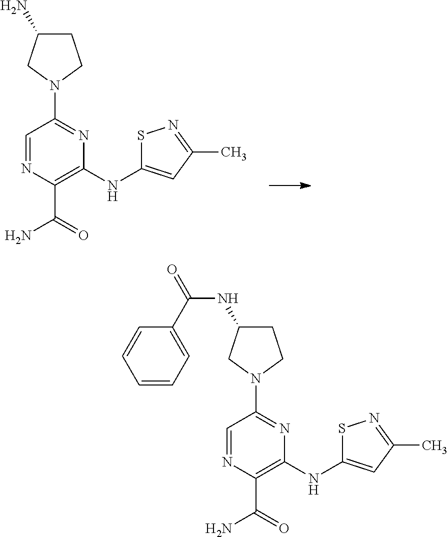 [SMILES: Cc1cc(Nc2nc(N3CC[C@@H](N)C3)cnc2C(N)=O)sn1 ; Cc1cc(Nc2nc(N3CC[C@@H](NC(=O)c4ccccc4)C3)cnc2C(N)=O)sn1]

In a similar manner as described in Example A-7, 5-[(3R)-3-benzamidopyrrolidin-1-yl]-3-[(3-methyl-1,2-thiazol-5-yl) amino]pyrazine-2-carboxamide (50) was prepared using benzoyl chloride. MS found for C20H21N7O2S as (M+H)⁺ 423.9. ¹H NMR (500 MHz, DMSO) δ 12.22 (s, 1H), 8.69 (d, J=6.85 Hz, 1H), 7.88 (d, J=7.34 Hz, 3H), 7.61-7.40 (m, 5H), 6.86 (s, 1H), 4.81-4.62 (m, 1H), 4.23-3.42 (m, 4H), 2.29 (s, 3H), 2.39-2.26 (m, 1H), 2.21-2.10 (m, 1H).

### Example A-51: 5-[(3R)-3-[(dimethylcarbamoyl)amino]pyrrolidin-1-yl]-3-[(3-methyl-1,2-thiazol-5-yl)amino]pyrazine-2-carboxamide (51)

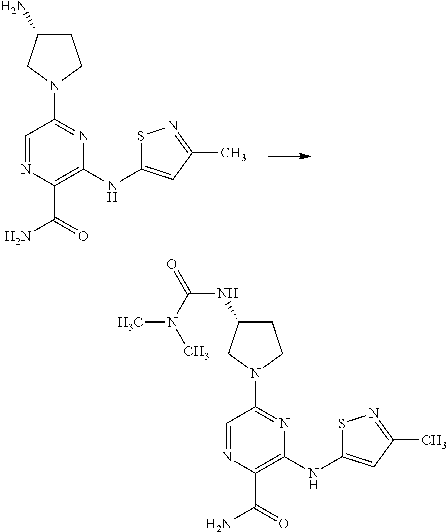

In a similar manner as described in Example A-7, 5-[(3R)-3-[(dimethylcarbamoyl)amino]pyrrolidin-1-yl]-3-[(3-methyl-1,2-thiazol-5-yl)amino]pyrazine-2-carboxamide (51) was prepared using dimethylcarbamyl chloride. MS found for C16H22N8O2S as (M+H)⁺ 391.0. ¹H NMR (500 MHz, DMSO) δ 12.20 (s, 1H), 7.85 (br. s., 1H), 7.56-7.34 (m, 2H), 6.84 (s, 1H), 6.41 (d, J=6.36 Hz, 1H), 4.47-4.23 (m, 1H), 4.09-3.49 (m, 4H), 2.80 (s, 6H), 2.28 (s, 3H), 2.24-2.14 (m, 1H), 2.09-1.95 (m, 1H).

### Example A-52: 5-[(3R)-3-[3-(3-chlorophenyl)-2-oxoimidazolidin-1-yl]piperidin-1-yl]-3-[(3-methyl-1,2-thiazol-5-yl)amino]pyrazine-2-carboxamide (52)

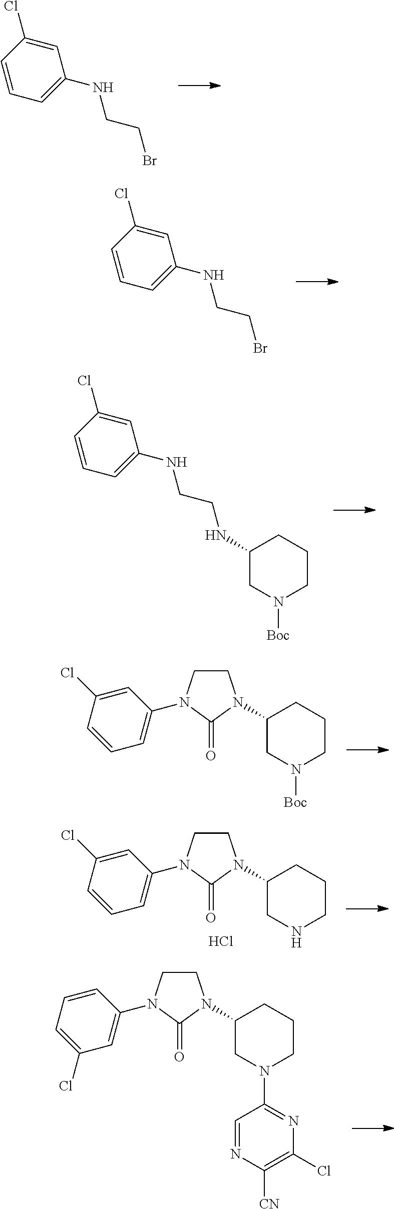

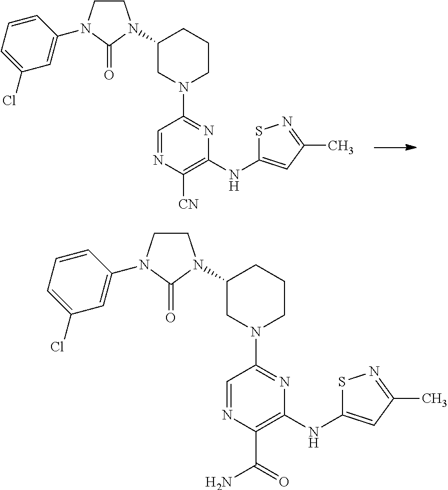

A mixture of 3-chloroaniline (50 g, 0.39 mol) and 1,2-dibromoethane (73 g, 0.39 mol) in MeCN (500 mL) was heated at 80° C. for 2 days. The reaction mixture was concentrated and subjected to silica flash column chromatography using 0 to 30% ethyl acetate in petroleum ether to isolate N-(2-bromoethyl)-3-chloroaniline (9.0 g, 10% yield) as a yellow solid. To a solution of N-(2-bromoethyl)-3-chloroaniline (9.0 g, 38.46 mmol) and (R)-tert-butyl 3-aminopiperidine-1-carboxylate (7.74 g, 38.46 mmol) in THF (100 mL) was added DIEA (14.88 g, 115.38 mmol). The resulting mixture was stirred at 70° C. for 2 days. It was partitioned between ethyl acetate (50 mL) and water (50 mL). The layers were separated and the aqueous was extracted with ethyl acetate (50 mL×3). The combined organic layers were washed with brine, dried, concentrated and subjected to silica flash column chromatography using 0 to 5% MeOH in DCM to isolated (R)-tert-butyl 3-(2-(3-chlorophenylamino)ethylamino)piperidine-1-carboxylate (10.1 g, 74.4% yield) as a white solid.

In an ice bath and under N₂ atmosphere, to a stirred solution of (R)-tert-butyl 3-(2-(3-chlorophenylamino)ethylamino)piperidine-1-carboxylate (10.1 g, 28.37 mmol) and Et₃N (3.01 g, 29.8 mmol) in anhydrous DCM (300 mL) was added a solution of triphosgene (2.95 g, 9.93 mmol) in DCM (100 mL) dropwise. The resulting mixture was stirred in an ice bath for 1 hour, and quenched with NaHCO₃ aqueous solution (1M, 50 mL). The mixture was extracted with DCM (100 mL×3). The combined organic layer was washed with brine, dried, concentrated and subjected to silica flash column chromatography using 0 to 5% MeOH in DCM to isolate (R)-tert-butyl 3-(3-(3-chlorophenyl)-2-oxoimidazolidin-1-yl) piperidine-1-carboxylate (4 g, 37% yield) as a white solid. It was treated with 40 mL commercial 4N HCl in dioxane to give (R)-1-(3-chlorophenyl)-3-(piperidin-3-yl)imidazolidin-2-one hydrochloride (3.3 g, quant. yield) as a white solid.

To a solution of 3,5-dichloropyrazine-2-carbonitrile (72 mg 0.406 mmol) in 2 mL of DMF, (R)-1-(3-chlorophenyl)-3-(piperidin-3-yl)imidazolidin-2-one hydrochloride (125 mg, 0.447 mmol) and 0.14 ml of DIEA were added. The reaction was left stirring at room temperature for 1.5 h. The solution was diluted with ethyl acetate (60 mL) and washed with water. The organic layer was dried over Na₂SO₄, the solid was filtered out and the filtrate concentrated to give 3-chloro-5-[(3R)-3-[3-(3-chlorophenyl)-2-oxoimidazolidin-1-yl]piperidin-1-yl]pyrazine-2-carbonitrile (202 mg, quant. yield) as crude, used in the next step without further purification.

To a mixture of 3-[3-(3-chlorophenyl)-2-oxoimidazolidin-1-yl]piperidin-1-yl]pyrazine-2-carbonitrile (202 mg, 0.406 mmol), 5-amino-3-methyl-isothiazole hydrochloride (73 mg, 0.487 mmol), Cs₂CO₃ (403 mg, 1.23 mmol) in 8 mL of dry dioxane, BINAP(±) (51 mg, 0.081 mmol) and Pd(OAc)₂ (19.5 mg, 0.086 mmol) were added. The mixture was degassed bubbling N₂ for 10 minutes and then refluxed for 3 hours. It was cooled, diluted with water (45 ml) and extracted with ethyl acetate. The collected organic layers were dried over Na₂SO₄, filtered and concentrated. The crude was purified on flash chromatography (silica) eluting with ethyl acetate in DCM from 10 to 60%. The fractions containing the product were collected and concentrated in vacuo to give the desired compound 5-[(3R)-3-[3-(3-chlorophenyl)-2-oxoimidazolidin-1-yl]piperidin-1-yl]-3-[(3-methyl-1,2-thiazol-5-yl)amino]pyrazine-2-carbonitrile (70.5 mg, 35% yield).

5-[(3R)-3-[3-(3-chlorophenyl)-2-oxoimidazolidin-1-yl]piperidin-1-yl]-3-[(3-methyl-1,2-thiazol-5-yl)amino]pyrazine-2-carbonitrile (70 mg, 0.141 mmol) was dissolved in 8 mL of MeOH and 4 mmol of DMSO. A pallet of NaOH (172 mg) and 1.35 mL of H₂O₂ (30% in H₂O) was added and the mixture was stirred at room temperature for 20 hours. More NaOH (180 mg) and H₂O₂ (1 mL) were added and the mixture was stirred for 24 hours. Ethyl acetate was added and the organic solution was washed with NaHCO₃ aqueous solution and with water. The organic layer was dried over Na₂SO₄, filtered and concentrated. The crude obtained was purified flash chromatography (silica) eluting with ethyl acetate in DCM from 10 to 65% to give 5-[(3R)-3-[3-(3-chlorophenyl)-2-oxoimidazolidin-1-yl]piperidin-1-yl]-3-[(3-methyl-1,2-thiazol-5-yl)amino]pyrazine-2-carboxamide (36 mg, 50% yield). MS found for C23H25ClN8O2S as (M+H)⁺ 513.0. ¹H NMR (400 MHz, DMSO) δ 12.29 (s, 1H), 7.93 (br. s., 1H), 7.86 (s, 1H), 7.83-7.79 (m, 1H), 7.56 (br. s., 1H), 7.47-7.39 (m, 1H), 7.34 (t, J=7.40 Hz, 1H), 7.05 (d, J=7.43 Hz, 1H), 6.85 (s, 1H), 4.65-4.42 (m, 2H), 3.94-3.69 (m, 3H), 3.67-3.50 (m, 2H), 3.41-3.25 (m, 1H), 3.20-3.06 (m, 1H), 2.28 (s, 3H), 2.00-1.81 (m, 3H), 1.76-1.57 (m, 1H).

### Example A-53: 5-[(3R)-3-[3-(3-chlorophenyl)-2-oxoimidazolidin-1-yl]piperidin-1-yl]-3-[(4-methanesulfonylphenyl)amino]pyrazine-2-carboxamide (53)

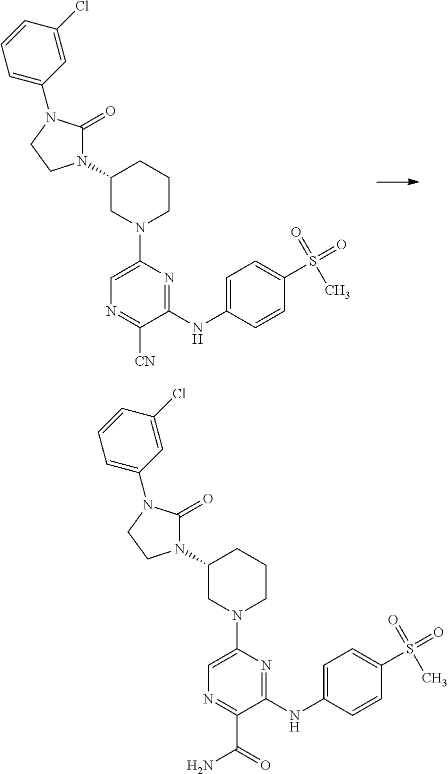 [SMILES: CS(=O)(=O)c1ccc(Nc2nc(N3CCC[C@@H](N4CCN(c5cccc(Cl)c5)C4=O)C3)cnc2C#N)cc1 ; CS(=O)(=O)c1ccc(Nc2nc(N3CCC[C@@H](N4CCN(c5cccc(Cl)c5)C4=O)C3)cnc2C(N)=O)cc1]

In a similar manner as described in Example A-52, 5-[(3R)-3-[3-(3-chlorophenyl)-2-oxoimidazolidin-1-yl]piperidin-1-yl]-3-[(4-methanesulfonylphenyl)amino]pyrazine-2-carboxamide (53) was prepared using 4-methanesulfonylaniline. MS found for C26H28ClN7O4S as (M+H)⁺ 570.1. ¹H NMR (500 MHz, DMSO) δ 11.88 (s, 1H), 7.92 (br. s, 1H), 7.89-7.85 (m, 2H), 7.84 (s, 1H), 7.83-7.79 (m, 2H), 7.71-7.66 (m, 1H), 7.61 (d, J=8.31 Hz, 1H), 7.52 (br. s, 1H), 7.37 (t, J=8.07 Hz, 1H), 7.05 (dd, J=8.07, 1.22 Hz, 1H), 4.56-4.24 (m, 2H), 3.89-3.83 (m, 2H), 3.82-3.74 (m, 1H), 3.65-3.52 (m, 2H), 3.24-3.16 (m, 1H), 3.06 (s, 3H), 3.10-2.99 (m, 1H), 2.00-1.54 (m, 4H).

### Preparation of tert-butyl N-(2-methylpiperidin-3-yl)carbamate

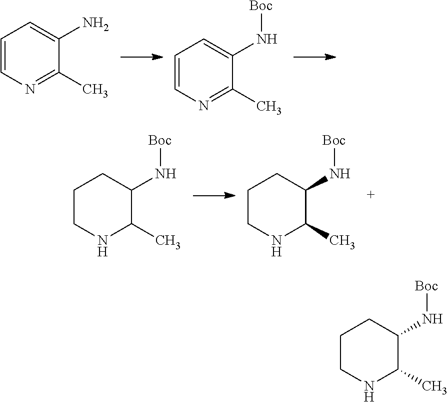

3-Amino-2-methylpyridine (3.11 g, 28.83 mmol) was dissolved in 60 mL of THF and di-tert-butyl dicarbonate (6.3 g, 28.83 mmol) were added. The solution was stirred for at room temperature for 2 weeks. The mixture was concentrated under vacuum and the crude purified by flash chromatography (silica) eluting with ethyl acetate in cyclohexane acetate from 50 to 100% The fractions containing the product were collected and concentrated in vacuum to give the desired compound tert-butyl N-(2-methylpyridin-3-yl)carbamate (5.41 g, 90% yield). Tert-butyl N-(2-methylpyridin-3-yl)carbamate (5.41 g, 25.99 mmol) was dissolved in AcOH (100 mL). PtO₂ (2.7 g) was added and the mixture was stirred overnight under H₂ atmosphere (4 atm). The catalyst was filtered off, the solvent was evaporated and the residue neutralized with K₂CO₃ solid. The mixture was extracted with ethyl acetate (3×250 mL) and DCM (3×100 mL). The combined organic phases was concentrated under reduced pressure to give tert-butyl N-(2-methylpiperidin-3-yl)carbamate (7.0 g, quant. yield) as diastereoisomeric mixture. The diastereoisomeric mixture was purified by preparative chiral HPLC to give: tert-butyl N-[(2S,3S)-2-methylpiperidin-3-yl]carbamate (1.94 g, 9.05 mmol), tert-butyl N-[(2R,3R)-2-methylpiperidin-3-yl]carbamate (1.39 g, 6.48 mmol).

### Example A-54: 5-[(2S,3S)-3-[4-(dimethylamino)benzamido]-2-methylpiperidin-1-yl]-3-[(3-methyl-1,2-thiazol-5-yl)amino]pyrazine-2-carboxamide (54)

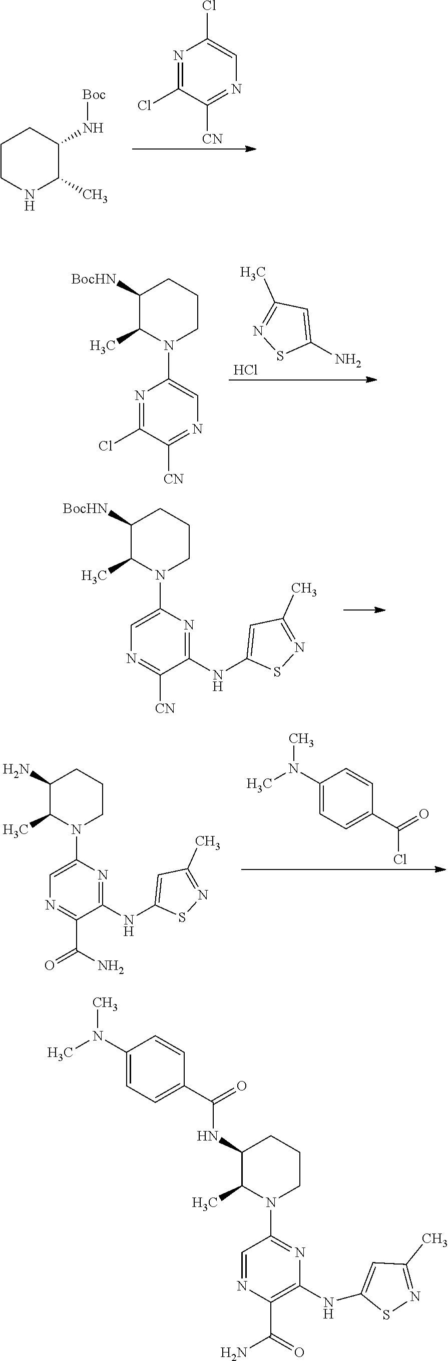

3,5-Dichloropyrazine-2-carbonitrile (487.14 mg, 2.8 mmol) was added to a solution of tert-butyl N-[(2S,3S)-2-methylpiperidin-3-yl]carbamate (600.0 mg, 2.8 mmol) and DIPEA (1.0 mL, 5.6 mmol) in DMF (5 mL) and stirred at room temperature for 2 hours. The mixture was poured into ice and extracted with ethyl acetate (3×50 mL), the organic phase dried on Na₂SO₄, filtered and concentrated under reduced pressure. The residue purified by flash chromatography (silica) eluting with ethyl acetate in cyclohexane from 0 to 60% to obtain tert-butyl N-[(2S,3S)-1-(6-chloro-5-cyanopyrazin-2-yl)-2-methylpiperidin-3-yl]carbamate (274.3 mg, 28% yield). To a mixture of tert-butyl N-[(2S,3S)-1-(6-chloro-5-cyanopyrazin-2-yl)-2-methylpiperidin-3-yl]carbamate (274.3 mg, 0.78 mmol) in 5 mL of 1,4-dioxane, 5-amino-3-methyl-isothiazole hydrochloride (117.43 mg, 1.559 mmol), Cs₂CO₃ (1015.2 mg, 3.116 mmol), BINAP(±) (97.01 mg, 0.1559 mmol) and Pd(OAc)₂ (35.0 mg, 0.1559 mmol) were added. The mixture was degassed bubbling N₂ for 10 minutes and then refluxed overnight. The mixture was diluted with 50 mL of ethyl acetate and filtered. The filtrate was concentrated and purified by flash chromatography (silica) eluting with ethyl acetate in cyclohexane from 0 to 100% to give tert-butyl N-[(2S,3S)-1-(5-cyano-6-[(3-methyl-1,2-thiazol-5-yl)amino]pyrazin-2-yl)-2-methylpiperidin-3-yl]carbamate (235.1 mg, 70% yield). This compound was dissolved in 5 mL of TFA and 0.5 mL of H₂SO₄ was added. The reaction was heated at 90° C. for 30 minutes. The reaction was concentrated under reduced pressure. The residue was filtered through SCX cartridge eluting with NH₃. 7 N in MeOH. The solution was concentrated in vacuo to give 5-[(2S,3S)-3-amino-2-methylpiperidin-1-yl]-3-[(3-methyl-,2-thiazol-5-yl)amino]pyrazine-2-carboxamide (91.1 mg, 48% yield).

4-Dimethylaminobenzoyl chloride (34.07 mg, 0.185 mmol) and DIPEA (0.114 mL, 0.654 mmol) were added to a solution of 5-[(2S,3S)-3-amino-2-methylpiperidin-1-yl]-3-[(3-methyl-1,2-thiazol-5-yl)amino]pyrazine-2-carboxamide (45 mg, 0.129 mmol) in 1.5 mL of DMF and the reaction was stirred at room temperature 8 hours. The mixture was concentrated and the residue purified by flash chromatography (silica) eluting with MeOH in DCM from 0 to 10% to give 5-[(2S,3S)-3-[4-(dimethylamino)benzamido]-2-methylpiperidin-1-yl]-3-[(3-methyl-1,2-thiazol-5-yl)amino]pyrazine-2-carboxamide (32.7 mg, 51% yield). MS found for C24H30N8O2S as (M+H)⁺ 495. ¹H NMR (500 MHz, DMSO) δ 12.28 (s, 1H), 8.05 (d, J=7.34 Hz, 1H), 7.91 (br. s., 1H), 7.82 (s, 1H), 7.79 (d, J=8.80 Hz, 2H), 7.55 (br. s., 1H), 6.83 (s, 1H), 6.72 (d, J=8.80 Hz, 2H), 5.49-4.77 (m, 1H), 4.75-4.24 (m, 1H), 4.15-3.90 (m, 1H), 3.16 (t, J=12.76 Hz, 1H), 2.98 (s, 6H), 2.27 (s, 3H), 2.10-1.51 (m, 4H), 1.21 (d, J=6.85 Hz, 3H).

### Preparation of 5-(3-[4-(dimethylamino)benzamido]-2-methylpiperidin-1-yl)-3-[(3-methyl-1,2-thiazol-5-yl)amino]pyrazine-2-carboxamide

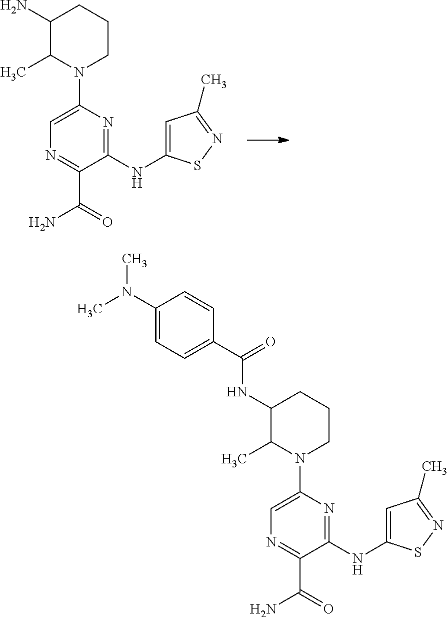

In a similar manner as described in Example A-54, 5-{3-[4-(dimethylamino)benzamido]-2-methylpiperidin-1-yl}-3-[(3-methyl-1,2-thiazol-5-yl)amino]pyrazine-2-carboxamide was prepared (81 mg, 0.163 mmol) and purified by preparative chiral HPLC giving:

### Example A-55: 5-[(2R,3R)-3-[4-(dimethylamino)benzamido]-2-methylpiperidin-1-yl]-3-[(3-methyl-1,2-thiazol-5-yl)amino]pyrazine-2-carboxamide (55)

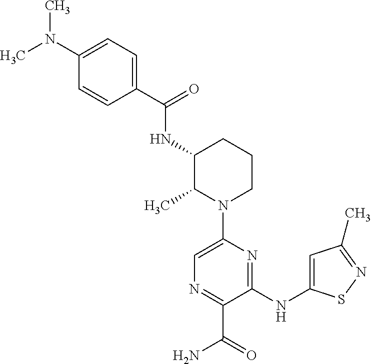 [SMILES: Cc1cc(Nc2nc(N3CCC[C@@H](NC(=O)c4ccc(N(C)C)cc4)[C@H]3C)cnc2C(N)=O)sn1]

(12.1 mg, 0.024 mmol, Y=21%). MS found for C24H30N8O2S as (M+H)⁺ 495.

¹H NMR (500 MHz, DMSO) δ 12.28 (s, 1H), 8.06 (d, J=7.34 Hz, 1H), 7.91 (br. s., 1H), 7.85-7.76 (m, 3H), 7.55 (br. s., 1H), 6.84 (s, 1H), 6.76-6.67 (m, 2H), 5.30-4.15 (m, 2H), 4.12-3.99 (m, 1H), 3.16 (t, J=12.96 Hz, 1H), 2.98 (d, J=1.47 Hz, 6H), 2.27 (s, 3H), 1.68 (d, J=13.69 Hz, 4H), 1.21 (d, J=6.85 Hz, 3H).

### Example A-56: 3-[(3-methyl-1,2-thiazol-5-yl)amino]-5-[(2R,3R)-2-methyl-3-[4-(propan-2-yl)benzamido]piperidin-1-yl]pyrazine-2-carboxamide (56)

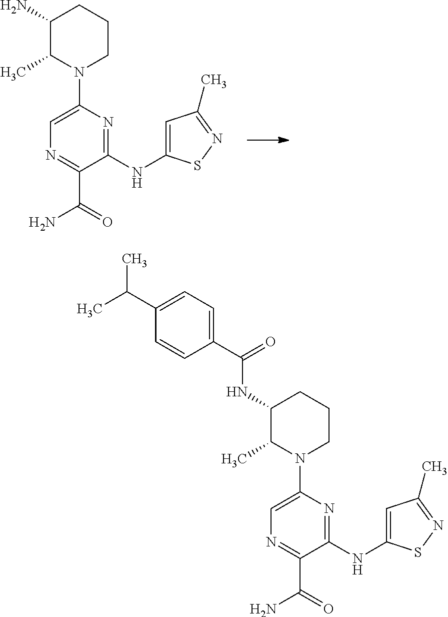

In a similar manner as described in Example A-8, 3-[(3-methyl-1,2-thiazol-5-yl)amino]-5-[(2R,3R)-2-methyl-3-[4-(propan-2-yl)benzamido]piperidin-1-yl]pyrazine-2-carboxamide (56) was prepared using 4-isopropylbenzoic acid. MS found for C25H31N7O2S as (M+H)⁺ 494. ¹H NMR (500 MHz, DMSO) δ 12.30 (s, 1H), 8.36 (d, J.=7.41 Hz, 1H), 8.11-7.69 (m, 4H), 7.56 (br. s., 1H), 7.35 (d, J=8.23 Hz, 2H), 6.85 (s, 1H), 5.50-4.73 (m, 1H), 4.52-4.04 (m, 2H), 3.26-2.88 (m, 2H), 2.27 (s, 3H), 2.03-1.84 (m, 2H), 1.78-1.55 (m, 2H), 1.32-1.19 (m, 9H).

### Example A-57: 5-[(2R,3R)-3-[(dimethylcarbamoyl)amino]-2-methylpiperidin-1-yl]-3-[(3-methyl-1,2-thiazol-5-yl)amino]pyrazine-2-carboxamide (57)

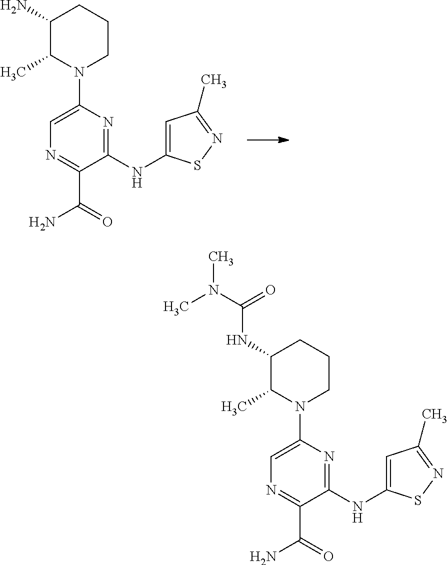 [SMILES: Cc1cc(Nc2nc(N3CCC[C@@H](N)[C@H]3C)cnc2C(N)=O)sn1 ; Cc1cc(Nc2nc(N3CCC[C@@H](NC(=O)N(C)C)[C@H]3C)cnc2C(N)=O)sn1]

In a similar manner as described in Example A-7, 5-[(2R,3R)-3-[(dimethylcarbamoyl)amino]-2-methylpiperidin-1-yl]-3-[(3-methyl-1,2-thiazol-5-yl)amino]pyrazine-2-carboxamide (57) was prepared using dimethylcarbamyl chloride. MS found for C18H26N8O2S as (M+H)⁺ 419. ¹H NMR (500 MHz, DMSO) δ 12.30 (s, 1H), 7.95-7.85 (m, 1H), 7.78 (s, 1H), 7.63-7.49 (m, 1H), 6.85 (s, 1H), 6.14 (d, J=6.85 Hz, 1H), 5.06-4.13 (m, 2H), 3.78-3.61 (m, 1H), 3.11 (td, J=13.08, 2.20 Hz, 1H), 2.82 (s, 6H), 2.37-2.23 (m, 3H), 1.96-1.45 (m, 4H), 1.17 (d, J=6.85 Hz, 3H).

### Example A-58: 5-[(2S,3S)-3-[(dimethylcarbamoyl)amino]-2-methylpiperidin-1-yl]-3-[(3-methyl-1,2-thiazol-5yl)amino]pyrazine-2-carboxamide (58)

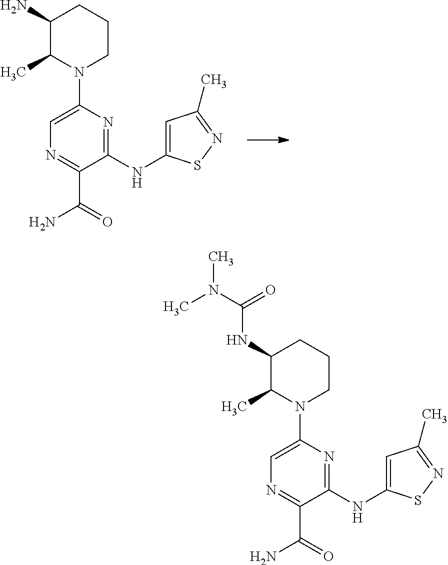 [SMILES: Cc1cc(Nc2nc(N3CCC[C@H](N)[C@@H]3C)cnc2C(N)=O)sn1 ; Cc1cc(Nc2nc(N3CCC[C@H](NC(=O)N(C)C)[C@@H]3C)cnc2C(N)=O)sn1]

In a similar manner as described in Example A-7, 5-[(2S,3S)-3-[(dimethylcarbamoyl)amino]-2-methylpiperidin-1-yl]-3-[(3-methyl-1,2-thiazol-5-yl)amino]pyrazine-2-carboxamide (58) was prepared using dimethylcarbamyl chloride. MS found for C18H26N8O2S as (M+H)⁺ 419. ¹H NMR (500 MHz, DMSO) δ 12.21 (s, 1H), 7.89 (br. s., 1H, 1H), 7.77 (s, 1H), 7.53 (br. s., 1H), 7.77 (d, J=1=8.78 Hz, 2H), 6.83 (s, 1H), 6.13 (d, J=7.04 Hz, 1H), 5.06-4.66 (m, 1H), 3.80-3.62 (m, 1H), 3.11 (t, J=12.91 Hz, 1H), 2.82 (s, 6H), 2.29 (s, 3H), 1.95-1.45 (m, 4H), 1.17 (m, J=6.65 Hz, 3H).

### Preparation of 5-{3-[(dimethylcarbamoyl)amino]-2-methylpiperidin-1-yl}-3-[(quinolin-6-yl)amino]pyrazine-2-carboxamide

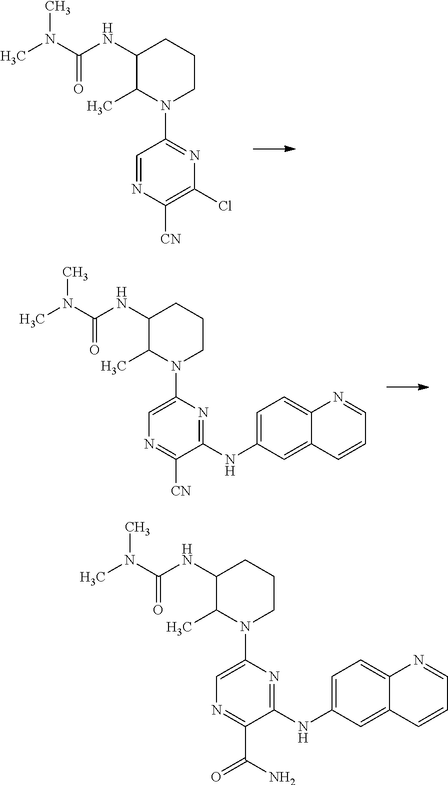 [SMILES: CC1C(NC(=O)N(C)C)CCCN1c1cnc(C#N)c(Cl)n1 ; CC1C(NC(=O)N(C)C)CCCN1c1cnc(C#N)c(Nc2ccc3ncccc3c2)n1 ; CC1C(NC(=O)N(C)C)CCCN1c1cnc(C(N)=O)c(Nc2ccc3ncccc3c2)n1]

To a mixture of 1-[1-(6-chloro-5-cyanopyrazin-2-yl)-2-methylpiperidin-3-yl]-3,3-dimethylurea (100.0 mg, 0.309 mmol) in 25 mL of 1,4-dioxane, quinolin-6-amine (89.33 mg, 0.619 mmol), Cs₂CO₃ (302.03 mg, 0.927 mmol), BINAP(±) (38.54 mg, 0.0619 mmol) and Pd(OAc)₂ (14.0 mg, 0.0619 mmol) were added. The mixture was degassed bubbling N₂ for 10 minutes and then heated at 100° C. overnight. The mixture was diluted with 50 mL of ethyl acetate and filtered. The filtrate was concentrated and purified by flash chromatography (silica) eluting with ethylacetate in cyclohexane from 10 to 100% to give 1-(1-{5-cyano-6-[(quinolin-6-yl)amino]pyrazin-2-yl}-2-methylpiperidin-3-yl)-3,3-dimethylurea (56.0 mg, 42% yield). 1-(1-{5-cyano-6-[(quinolin-6-yl)amino]pyrazin-2-yl}-2-methylpiperidin-3-yl)-3,3-dimethylurea (56.0 mg, 0.13 mmol) was dissolved in a mixture of MeOH/DMSO (2:1). 1 mL of TEA and 0.1 mL of H₂O₂ (30% in water) were added. The mixture was stirred at room temperature for 48 h, then diluted with ethyl acetate and washed with water. The organic phase was dried on Na₂SO₄, filtered and concentrated under reduced pressure. The residue was purified by preparative chiral HPLC to give:

### Example A-59: 5-[(2R,3R)-3-[(dimethylcarbamoyl)amino]-2-methylpiperidin-1-yl]-3-[(quinolin-6-yl)amino]pyrazine-2-carboxamide (59)

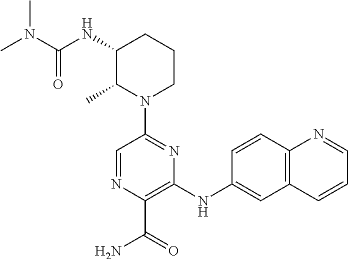 [SMILES: C[C@@H]1[C@H](NC(=O)N(C)C)CCCN1c1cnc(C(N)=O)c(Nc2ccc3ncccc3c2)n1]

16 mg, 0.037 mmol; MS found for C23H28N8O2 as (M+H)⁺ 449. ¹H NMR (500 MHz, DMSO) δ 11.77 (s, 1H), 8.74-8.70 (m, 1H), 8.56-8.49 (m, 1H), 8.25 (d, J=8.31 Hz, 1H), 7.94 (d, J=9.29 Hz, 1H), 7.89-7.83 (m, 1H), 7.73 (s, 1H), 7.69-7.63 (m, 1H), 7.50-7.45 (m, 1H), 7.44-7.39 (m, 1H), 6.21 (d, J=6.85 Hz, 1H), 5.39-4.91 (m, 1H), 4.31-3.95 (m, 1H), 3.90-3.68 (m, 1H), 3.06 (t, J=13.21 Hz, 1H), 2.89 (s, 6H), 2.29 (s, 3H), 2.04-1.44 (m, 4H), 1.09 (d, J=6.85 Hz, 3H).

### Example A-60: 5-[(2S,3S)-3-[(dimethylcarbamoyl)amino]-2-methylpiperidin-1-yl]-3-[(quinolin-6-yl)amino]pyrazine-2-carboxamide (60)

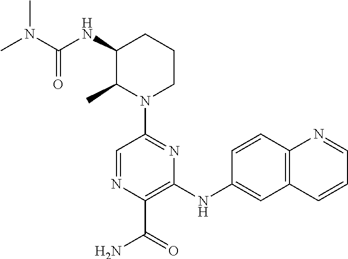 [SMILES: C[C@H]1[C@@H](NC(=O)N(C)C)CCCN1c1cnc(C(N)=O)c(Nc2ccc3ncccc3c2)n1]

17 mg, 0.038 mmol; MS found for C23H28N8O2 as (M+H)⁺ 449. ¹H NMR (500 MHz, DMSO) δ 11.77 (s, 1H), 8.74-8.70 (m, 1H), 8.56-8.49 (m, 1H), 8.25 (d, J=8.31 Hz, 1H), 7.94 (d, J=9.29 Hz, 1H), 7.89-7.83 (m, 1H), 7.73 (s, 1H), 7.69-7.63 (m, 1H), 7.50-7.45 (m, 1H), 7.44-7.39 (m, 1H), 6.21 (d, J=6.85 Hz, 1H), 5.39-4.91 (m, 1H), 4.31-3.95 (m, 1H), 3.90-3.68 (m, 1H), 3.06 (t, J=13.21 Hz, 1H), 2.89 (s, 6H), 2.29 (s, 3H), 2.04-1.44 (m, 4H), 1.09 (d, J=6.85 Hz, 3H).

### Example A-61: 5-[(3R)-3-benzamidopiperidin-1-yl]-3-[(3-methyl-1,2-thiazol-5-yl)amino]pyridine-2-carboxamide (61)

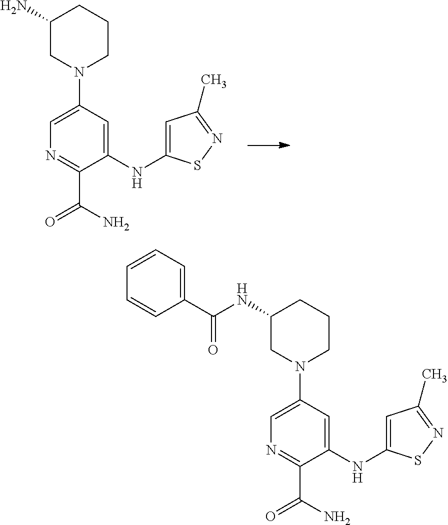 [SMILES: Cc1cc(Nc2cc(N3CCC[C@@H](N)C3)cnc2C(N)=O)sn1 ; Cc1cc(Nc2cc(N3CCC[C@@H](NC(=O)c4ccccc4)C3)cnc2C(N)=O)sn1]

In a similar manner as described in Example A-7, 5-[(3R)-3-benzamidopiperidin-1-yl]-3-[(3-methyl-1,2-thiazol-5-yl)amino]pyridine-2-carboxamide (61) was prepared using benzoyl chloride. MS found for C22H24N6O2S as (M+H)⁺ 437.0. ¹H NMR (500 MHz, DMSO) δ 12.03 (s, 1H), 8.41 (d, J=6.85 Hz, 1H), 8.14-8.00 (m, 1H), 7.99-7.94 (m, 1H), 7.85 (d, J=7.34 Hz, 2H), 7.65-7.56 (m, 1H), 7.53 (m, J=7.34 Hz, 1H), 7.49-7.44 (m, 2H), 7.03-6.94 (m, 1H), 6.93-6.86 (m, 1H), 4.11-3.74 (m, 3H), 3.12-3.01 (m, 2H), 2.32 (s, 3H), 2.02-1.92 (m, 1H), 1.91-1.79 (m, 1H), 1.79-1.68 (m, 1H), 1.68-1.57 (m, 1H).

### Example A-62: 5-[(3R)-3-[4-(dimethylamino)benzamido]piperidin-1-yl]-3-[(3-methyl-1,2-thiazol-5-yl)amino]pyridine-2-carboxamide (62)

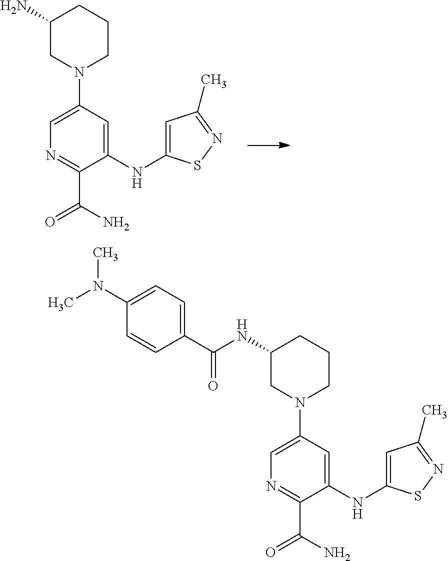

In a similar manner as described in Example A-7, 5-[(3R)-3-[4(dimethylamino)benzamido]piperidin-1-yl]-3-[(3-methyl-1,2-thiazol-5-yl)amino]pyridine-2-carboxamide (62) was prepared using 4-(dimethylamino) benzoyl chloride. MS found for C24H29N7O2S as (M+H)⁺ 480.0. ¹H NMR (500 MHz, DMSO) δ 12.03 (s, 1H), 8.13-7.99 (m, 2H), 7.96 (d, J=2.45 Hz, 1H), 7.77 (d, J=8.80 Hz, 2H), 7.67-7.50 (m, 1H), 6.98 (d, J=2.45 Hz, 1H), 6.91 (s, 1H), 6.82-6.72 (m, 2H), 4.07-3.80 (m, 3H), 2.98 (s, 6H), 3.11-2.86 (m, 2H), 2.33 (s, 3H), 2.00-1.90 (m, 1H), 1.88-1.79 (m, 1H), 1.77-1.67 (m, 1H), 1.66-1.54 (m, 1H).

### Example A-63: 3-[(3-methyl-1,2-thiazol-5-yl)amino]-5-[(3R)-3-[4-(propan-2-yl)benzamido]piperidin-1-yl]pyridine-2-carboxamide (63)

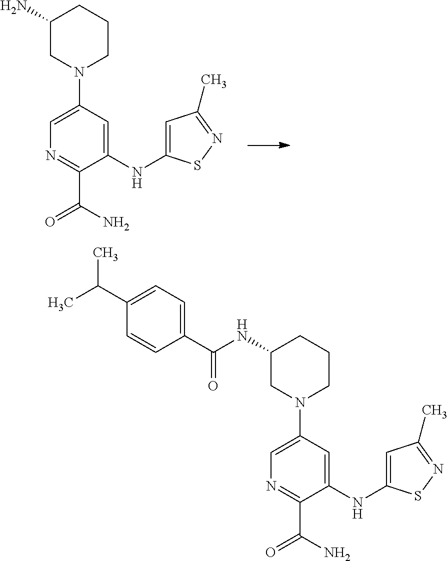

In a similar manner as described in Example A-8, 3-[(3-methyl-1,2-thiazol-5-yl)amino]-5-[(3R)-3-[4-(propan-2-yl)benzamido]piperidin-1-yl]pyridine-2-carboxamide (63) was prepared using 4-isopropylbenzoic acid. MS found for C25H30N6O2S as (M+H)⁺ 479.1. ¹H NMR (500 MHz, DMSO) δ 12.03 (s, 1H), 8.30 (d, J=6.86 Hz, 1H), 8.04 (d, J=2.33 Hz, 1H), 7.95 (d, J=2.33 Hz, 1H), 7.78 (d, J=8.40 Hz, 2H), 7.57 (d, J=2.30 Hz, 1H), 7.33 (d, J=8.37 Hz, 2H), 6.97 (d, J=2.33 Hz, 1H), 6.89 (s, 1H), 4.02-3.83 (m, 3H), 3.12-2.90 (m, 3H), 2.32 (s, 3H), 2.02-1.91 (m, 1H), 1.89-1.80 (m, 1H), 1.78-1.55 (m, 2H), 1.21 (d, J=7.00 Hz, 6H).

### Example A-64: 5-[(3R)-3-[(dimethylcarbamoyl)amino]piperidin-1-yl]-3-[(3-methyl-1,2-thiazol-5-yl)amino]pyridine-2-carboxamide (64)

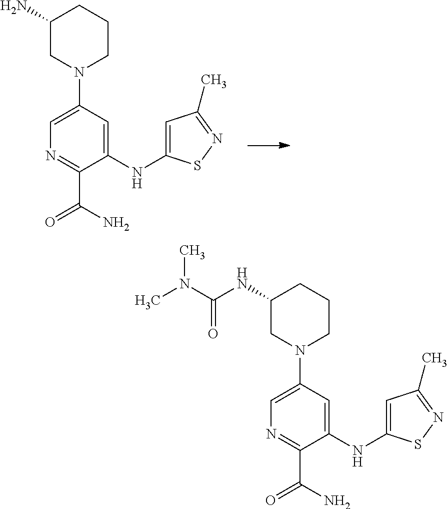 [SMILES: Cc1cc(Nc2cc(N3CCC[C@@H](N)C3)cnc2C(N)=O)sn1 ; Cc1cc(Nc2cc(N3CCC[C@@H](NC(=O)N(C)C)C3)cnc2C(N)=O)sn1]

In a similar manner as described in Example A-7, 5-[(3R)-3 [(dimethylcarbamoyl)amino]piperidin-1-yl]-3-[(3-methyl-1,2-thiazol-5-yl)amino]pyridine-2-carboxamide (64) was prepared using dimethylcarbamyl chloride. MS found for C18H25N7O2S as (M+H)⁺ 404.1. ¹H NMR (500 MHz, DMSO) δ 12.03 (s, 1H), 8.02 (br. s., 1H), 7.92 (d, J=2.45 Hz, 1H), 7.55 (br. s., 1H), 6.93 (d, J=2.45 Hz, 1H), 6.90 (s, 1H), 6.08 (d, J=6.85 Hz, 1H), 3.96-3.82 (m, 2H), 3.63-3.52 (m, 1H), 3.02-2.93 (m, 1H), 2.86-2.74 (m, 7H), 2.33 (s, 3H), 1.92-1.84 (m, 1H), 1.81-1.73 (m, 1H), 1.66-1.48 (m, 2H).

### Example A-65: Synthesis of 3-(phenylamino)-5-[(3R)-3-[4-(propan-2-yl)benzamido]piperidin-1-yl]pyrazine-2-carboxamide

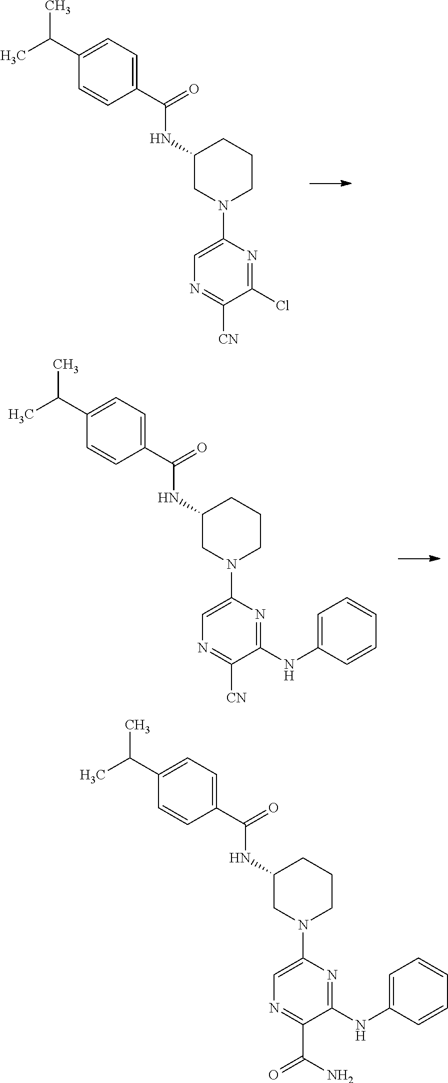 [SMILES: CC(C)c1ccc(C(=O)N[C@@H]2CCCN(c3cnc(C#N)c(Cl)n3)C2)cc1 ; CC(C)c1ccc(C(=O)N[C@@H]2CCCN(c3cnc(C#N)c(Nc4ccccc4)n3)C2)cc1 ; CC(C)c1ccc(C(=O)N[C@@H]2CCCN(c3cnc(C(N)=O)c(Nc4ccccc4)n3)C2)cc1]

In a similar manner as described in Example A-8, N-[(3R)-1-(6-chloro-5-cyanopyrazin-2-yl)piperidin-3-yl]-4-(propan-2-yl)benzamide was prepared using 4-(propan-2-yl)benzoic acid. MS found for C20H22ClN5O as (M+H)⁺ 384.3, (M−H)⁻ 382.3. In a similar manner as described in Example A-1, N-[(3R)-1-[5-cyano-6-(phenylamino)pyrazin-2-yl]piperidin-3-yl]-4-(propan-2-yl)benzamide was prepared using aniline and N-[(3R)-1-(6-chloro-5-cyanopyrazin-2-yl)piperidin-3-yl]-4-(propan-2-yl)benzamide. MS found for C26H28N6O as (M+H)⁺ 441.0. In a similar manner as described in Example A-1, 3-(phenylamino)-5-[(3R)-3-[4-(propan-2-yl)benzamido]piperidin-1-yl]pyrazine-2-carboxamide (65) was prepared using N-[(3R)-1-[5-cyano-6-(phenylamino)pyrazin-2-yl]piperidin-3-yl]-4-(propan-2-yl)benzamide. MS found for C26H30N6O2 as (M+H)⁺ 459.0. ¹H NMR (400 MHz, DMSO) δ 11.32 (s, 1H), 8.31 (s, 1H), 7.87-7.74 (m, 3H), 7.70 (s, 1H), 7.61 (d, J=7.89 Hz, 2H), 7.34 (d, J=8.11 Hz, 3H), 7.26 (t, J=7.78 Hz, 2H), 6.95 (t, J=7.80 Hz, 1H), 4.48-4.37 (m, 1H), 4.27-4.14 (m, 1H), 4.06-3.88 (m, 1H), 3.24-3.03 (m, 2H), 3.01-2.89 (m, 1H), 2.03-1.93 (m, 1H), 1.93-1.82 (m, 1H), 1.79-1.67 (m, 1H), 1.66-1.51 (m, 1H), 1.22 (d, J=7.02 Hz, 6H).

### Example A-66: Synthesis of 5-[(3R)-3-[4-(dimethylamino)benzamido]piperidin-1-yl]-4-methyl-3-[(3-methyl-1,2-thiazol-5-yl)amino]pyridine-2-carboxamide (66)

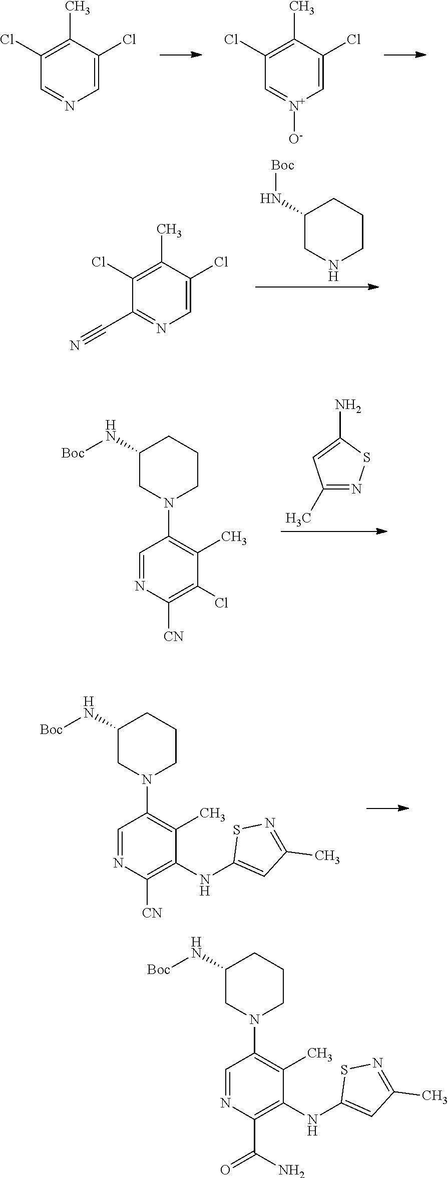

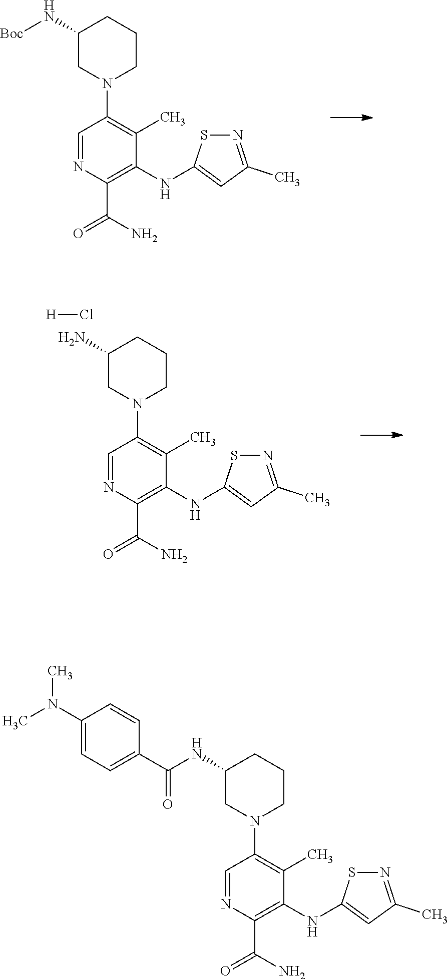

3,5-Dichloro-4-Picoline (2.0 g, 12.34 mmol) was dissolved in dry CH₂Cl₂ (30 mL), mCPBA (2.788 g, 16.10 mmol) was added and the reaction mixture was stirred at room temperature overnight. K₂CO₃ was added (1.771 g) and the mixture was stirred at room temperature for 1 hour. The solid was filtered off and the organic phase was concentrated to give 3,5-dichloro-4-methylpyridin-1-ium-1-olate (1.892 g, 86% yield) as white solid. MS found for C6H5C12NO as (M+H)⁺ 177.9.

3,5-dichloro-4-methylpyridin-1-ium-1-olate (1.892 g, 10.63 mmol) was dissolved in ACN (35 mL), Et₃N (2.22 mL) and TMSCN (2.66 mL, 21.256 mmol) was added at room temperature. The reaction mixture was refluxed for 7 hours and stirred for further 10 hours at room temperature. Ethyl acetate (200 mL) was added and the mixture was washed with aqueous NaHCO₃ (100 mL) and brine (100 mL). The organic phase was separated and concentrated under reduced pressure to obtain 3,5-dichloro-4-methylpyridine-2-carbonitrile 3,5-dichloro-4-methylpyridine-2-carbonitrile (1.678 g, 84% yield) as brown-red liquid. MS found for C7H4Cl2N2 as (M+H)⁺ 186.9.

In a similar manner as described in Example A-1, tert-butyl N-[(3R)-1-(5-chloro-6-cyano-4-methylpyridin-3-yl)piperidin-3-yl]carbamate was prepared using 3,5-dichloro-4-methylpyridine-2-carbonitrile. MS found for C17H23ClN4O2 as (M+H)⁺ 351.0.

In a similar manner as described in Example A-1, tert-butyl N-[(3R)-1-{6-cyano-4-methyl-5-[(3-methyl-1,2-thiazol-5-yl)amino]pyridin-3-yl}piperidin-3-yl]carbamate (66.5) was prepared using tert-butyl N-[(3R)-1-(5-bromo-6-cyanopyridin-3-yl)piperidin-3-yl]carbamate. MS found for C21H28N6O2S as (M+H)⁺ 429.0.

In a similar manner as described in Example A-1, tert-butyl N-[(3R)-1-(6-carbamoyl-4-methyl-5-[(3-methyl-1,2-thiazol-5-yl)amino]pyridin-3-yl) piperidin-3-yl]carbamate was prepared using tert-butyl N-[(3R)-1-{6-cyano-4-methyl-5-[(3-methyl-1,2-thiazol-5-yl)amino]pyridin-3-yl}piperidin-3-yl]carbamate. MS found for C21H30N6O3S as (M+H)⁺ 447.4.

In a similar manner as described in Example A-1, 5-[(3R)-3-aminopiperidin-1-yl]-4-methyl-3-[(3-methyl-1,2-thiazol-5-yl)amino]pyridine-2-carboxamide hydrochloride was prepared using tert-butyl N-[(3R)-1-(5-carbamoyl-6-{[4-(1-cyclopentylpiperidin-4-yl)phenyl]amino}pyrazin-2-yl)piperidin-3-yl]carbamate. MS found for C16H23ClN6OS as (M+H)⁺ 347.0.

In a similar manner as described in Example A-7, 5-[(3R)-3-[4-(dimethylamino)benzamido]piperidin-1-yl]-4-methyl-3-[(3-methyl-1,2-thiazol-5-yl)amino]pyridine-2-carboxamide (66) was prepared using 4-(dimethylamino)benzoyl chloride. MS found for C25H31N7O2S as (M+H)⁺ 494.2. ¹H NMR (400 MHz, DMSO) δ 10.30 (s, 1H), 8.15 (d, J=1.92 Hz, 1H), 8.09 (s, 1H), 7.94 (d, J=7.41 Hz, 1H), 7.76-7.71 (m, 2H), 7.68 (d, J=1.92 Hz, 1H), 6.72-6.66 (m, 2H), 6.23 (s, 1H), 4.11-3.99 (m, 1H), 3.44-3.37 (m, 1H), 3.28-3.21 (m, 1H), 2.96 (s, 6H), 2.90-2.83 (m, 1H), 2.80-2.72 (m, 1H), 2.23 (s, 3H), 2.12 (s, 3H), 1.98-1.84 (m, 2H), 1.76-1.56 (m, 2H).

### Preparation of 5-[3-[4-(dimethylamino)benzamido]-2-(hydroxymethyl)piperidin-1-yl]-3-[(3-methyl-1,2-thiazol-5-yl)amino]pyrazine-2-carboxamide

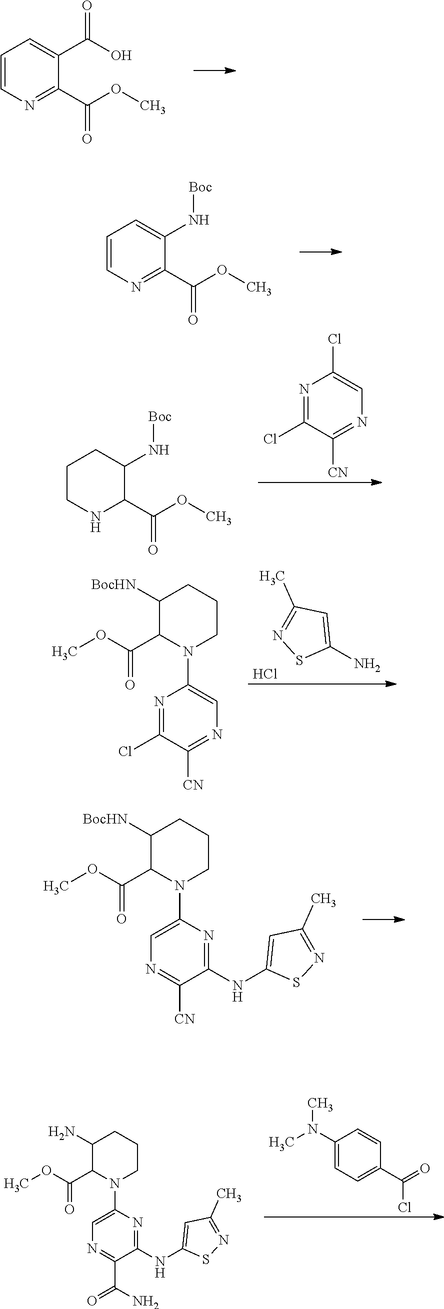

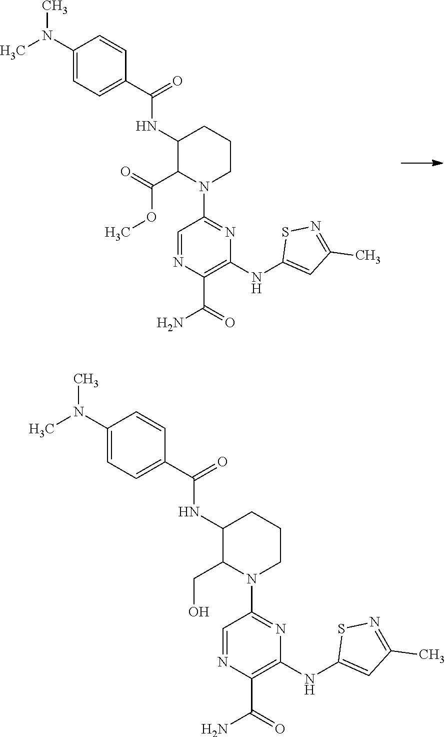

2-(Methoxycarbonyl)pyridine-3-carboxylic acid (3 g, 16.57 mmol) was dissolved in 50 mL of t-ButOH and 4 mL of TEA were added. The solution was stirred for 5 min at room temperature, then diphenylphosphorylazide (3.6 mL, 16.57 mmol) was added and the reaction was refluxed for 3 hours. The mixture was concentrated and the residue purified flash chromatography (silica) eluting with ethyl acetate in cyclohexane from 20 to 50%/. The fractions containing the product were collected and concentrated in vacuo to give methyl 3-{[(tert-butoxy)carbonyl]amino}pyridine-2-carboxylate (1.6 g, 38.5% yield).

Methyl 3-{[(tert-butoxy)carbonyl]amino}pyridine-2-carboxylate (1.53 g, 6.06 mmol) was dissolved in AcOH (30 mL). PtO₂ (770 mg) was added and the mixture was stirred under H₂ atmosphere (5 bar) for 12 hours. The catalyst was filtered off, the solvent was evaporated and the residue taken up with DCM (50 mL) and washed with NaHCO₃ aqueous saturated solution. The organic phase was concentrated to give methyl 3-{[(tert-butoxy)carbonyl]amino}piperidine-2-carboxylate (1.42 g, 91% yield) as diastereoisomeric mixture.

3,5-Dichloropyrazine-2-carbonitrile (1.15 g, 6.59 mmol) was added to a solution of methyl 3-{[(tert-butoxy)carbonyl]amino}piperidine-2-carboxylate (1.42 g, 5.5 mmol) and DIPEA (1.9 mL, 11 mmol) in DMF (15 ml) and heated at 60° C. for 4 hours. The mixture was concentrated and the residue purified by flash chromatography (silica) eluting with ethyl acetate in cyclohexane 10% to 80% to obtain methyl 3-{[(tert-butoxy)carbonyl]amino}-1-(6-chloro-5-cyanopyrazin-2-yl)piperidine-2-carboxylate (1.94 g, 74% yield) as diastereoisomeric mixture.

To a mixture of methyl 3-{[(tert-butoxy)carbonyl]amino}-1-(6-chloro-5-cyanopyrazin-2-yl)piperidine-2-carboxylate (1.7 g, 4.29 mmol), 5-amino-3-methyl-isothiazole hydrochloride (1.94 g, 12.88 mmol), Cs₂CO₃ (4.2 g, 12.97 mmol), BINAP(±) (534 mg, 0.858 mmol) and Pd(OAc)₂ (192 mg, 0.858 mmol) were added. The mixture was degassed bubbling N₂ for 10 minutes and then refluxed for 5 hours. The mixture was concentrated, redissolved in DCM and filtered. The filtrate was concentrated and purified by flash chromatography (silica) eluting with ethyl acetate in cyclohexane 30 to 50% to give methyl 3-{[(tert-butoxy)carbonyl]amino}-1-{5-cyano-6-[(3-methyl-1,2-thiazol-5-yl)amino]pyrazin-2-yl}piperidine-2-carboxylate (1.432 g, 3.024 mmol). This compound was dissolved in 30 mL of TFA and 1 mL of H₂SO₄ was added. The reaction was left stirring 8 hours at room temperature. Na₂CO₃ aqueous saturated solution (2 mL) was added and the mixture was concentrated in vacuo. The residue was passed through SCX cartridge eluting with NH₃ 7 N in MeOH solution. The obtained solution was concentrated in vacuo to give methyl 3-amino-1-{5-carbamoyl-6-[(3-methyl-1,2-thiazol-5-yl)amino]pyrazine-2-yl}piperidine-2-carboxylate (1.092 g) as mixture of diastereoisomers.

4-Dimethylaminobenzoyl chloride (614.8 mg, 3.34 mmol) and DIPEA (1.5 mL, 8.37 mmol) were added to a solution of methyl 3-amino-1-{5-carbamoyl-6-[(3-methyl-1,2-thiazol-5-yl)amino]pyrazine-2-yl}piperidine-2-carboxylate (1.092 g) in 10 mL of DMF and the reaction was stirred at room temperature 8 hours. The mixture was concentrated and the residue purified by flash chromatography (silica) eluting with ethyl acetate in cyclohexane from 10 to 80% to give methyl 1-{5-carbamoyl-6-[(3-methyl-1,2-thiazol-5-yl)amino]pyrazin-2-yl}-3-[4-(dimethylamino)benzamido]piperidine-2-carboxylate (1.081 g).

To a solution of methyl 1-{5-carbamoyl-6-[(3-methyl-1,2-thiazol-5-yl)amino]pyrazin-2-yl}-3-[4-(dimethylamino) benzamido] piperidine-2-carboxylate (1.081 g, 2.006 mmol) was dissolved in THF (15 mL). To the solution LiAlH₄ 2M solution in THF (1.2 mL) was added dropwise. The mixture was left stirring at room temperature 12 hours. Further 0.5 mL of LiAlH₄ 2 M solution in THF was added and the reaction was stirred 4 hours. Na₂SO₄×10 H₂O was added portionwise, DCM was added and the solid was filtered off. The filtrate was concentrated and purified on by flash chromatography eluting with MeOH in DCM from 5 to 40% obtaining the two diasteroisomers: cis-5-[3-[4-(dimethylamino)benzamido]-2-(hydroxymethyl)piperidin-1-yl]-3-[(3-methyl-1,2-thiazol-5-yl)amino]pyrazine-2-carboxamide (430 mg) and trans-5-[3-[4-(dimethylamino)benzamido]-2-(hydroxymethyl)piperidin-1-yl]-3-[(3-methyl-1,2-thiazol-5-yl)amino]pyrazine-2-carboxamide (130 mg).

cis-5-[3-[4-(dimethylamino)benzamido]-2-(hydroxymethyl)piperidin-1-yl]-3-[(3-methyl-1,2-thiazol-5-yl)amino]pyrazine-2-carboxamide (430 mg) was purified by preparative chiral HPLC to give:

### Example A-67: 5-[(2R,3S)-3-[4-(dimethylamino)benzamido]-2-(hydroxymethyl)piperidin-1-yl]-3-[(3-methyl-1,2-thiazol-5-yl)amino]pyrazine-2-carboxamide (67)

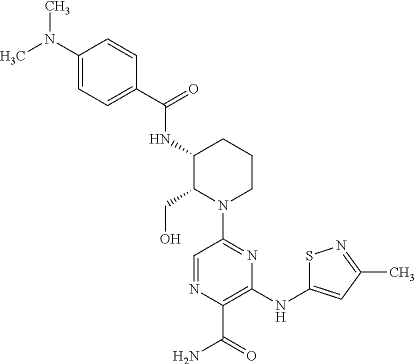

101 mg, 0.198 mmol; MS found for C24H30N8O3S as (M+H)⁺ 511. ¹H NMR (500 MHz, DMSO) δ 12.28 (s, 1H), 8.12 (d, J=7.14 Hz, 1H), 7.90 (br. s., 1H), 7.83 (s, 1H), 7.77 (d, J=8.78 Hz, 2H), 7.52 (br. s., 1H), 6.84 (s, 1H), 6.72 (d, J=9.06 Hz, 2H), 4.78 (t, J=5.21 Hz, 1H), 5.07-4.49 (m, 2H), 4.13-4.03 (m, 1H), 4.02-3.92 (m, 1H), 3.84-3.68 (m, 1H), 3.23 (t, J=12.76 Hz, 1H), 2.98 (s, 6H), 2.29 (s, 3H), 2.00-1.86 (m, 2H), 1.77-1.70 (m, 1H), 1.70-1.57 (m, 1H).

### Example A-68: 5-[(2S,3R)-3-[4-(dimethylamino)benzamido]-2-(hydroxymethyl)piperidin-1-yl]-3-[(3-methyl-1,2-thiazol-5-yl)amino]pyrazine-2-carboxamide (68)

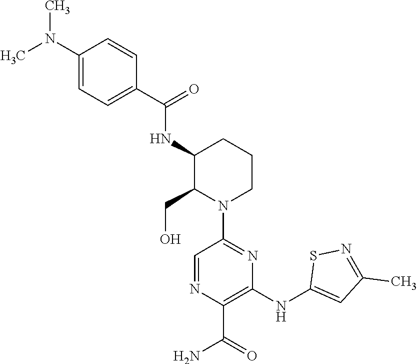

124 mg, 0.243 mmol; MS found for C24H30N8O3S as (M+H)⁺ 511. ¹H NMR (500 MHz, DMSO) δ 12.28 (s, 1H), 8.12 (d, J=7.14 Hz, 1H), 7.90 (br. s., 1H), 7.83 (s, 1H), 7.77 (d, J=8.78 Hz, 2H), 7.52 (br. s., 1H), 6.84 (s, 1H), 6.72 (d, J=9.06 Hz, 2H), 4.78 (t, J=5.21 Hz, 1H), 5.07-4.49 (m, 2H), 4.13-4.03 (m, 1H), 4.02-3.92 (m, 1H), 3.84-3.68 (m, 1H), 3.23 (t, J=12.76 Hz, 1H), 2.98 (s, 6H), 2.29 (s, 3H), 2.00-1.86 (m, 2H), 1.77-1.70 (m, 1H), 1.70-1.57 (m, 1H).

trans-5-[3-[4-(dimethylamino)benzamido]-2-(hydroxymethyl)piperidin-1-yl]-3-[(3-methyl-1,2-thiazol-5-yl)amino]pyrazine-2-carboxamide (430 mg) was purified by preparative chiral HPLC to give:

### Example A-69: 5-[(2R,3R)-3-[4-(dimethylamino)benzamido]-2-(hydroxymethyl)piperidin-1-yl]-3-[(3-methyl-1,2-thiazol-5-yl)amino]pyrazine-2-carboxamide (69)

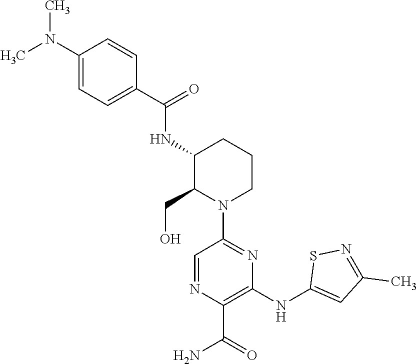

28 mg, 0.055 mmol; MS found for C24H30N8O3S as (M+H)⁺ 511.0. ¹H NMR (500 MHz, DMSO) δ 12.30 (s, 1H), 7.96 (d, J=7.14 Hz, 1H), 7.84 (s, 1H), 7.71 (s, 1H), 7.64 (d, J=9.06 Hz, 2H), 7.46 (br. s., 1H), 6.82 (s, 1H), 6.63 (d, J=9.06 Hz, 2H), 4.98 (t, J=5.35 Hz, 1H), 4.65 (br. s., 2H), 4.31 (br. s., 1H), 3.84-3.63 (m, 2H), 3.23 (td, J=12.97, 3.43 Hz, 1H), 2.92 (s, 6H), 2.29 (s, 3H), 2.12-1.97 (m, 1H), 1.97-1.84 (m, 1H), 1.76-1.61 (m, 2H).

### Example A-70: 5-[(2S,3S)-3-[4-(dimethylamino)benzamido]-2-(hydroxymethyl)piperidin-1-yl]-3-[(3-methyl-1,2-thiazol-5-yl)amino]pyrazine-2-carboxamide (70)

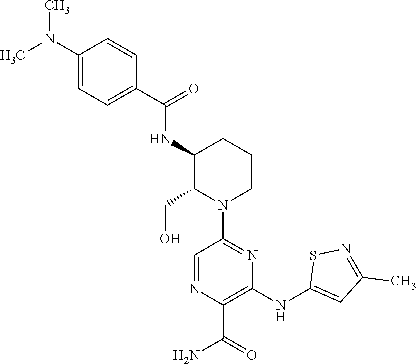

29 mg, 0.057 mmol; MS found for C24H30N8O3S as (M+H)⁺ 511.0. ¹H NMR (500 MHz, DMSO) δ 12.30 (s, 1H), 7.96 (d, J=7.14 Hz, 1H), 7.84 (s, 1H), 7.71 (s, 1H), 7.64 (d, J=9.06 Hz, 2H), 7.46 (br. s., 1H), 6.82 (s, 1H), 6.63 (d, J=9.06 Hz, 2H), 4.98 (t, J=5.35 Hz, 1H), 4.65 (br. s., 2H), 4.31 (br. s., 1H), 3.84-3.63 (m, 2H), 3.23 (td, J=12.97, 3.43 Hz, 1H), 2.92 (s, 6H), 2.29 (s, 3H), 2.12-1.97 (m, 1H), 1.97-1.84 (m, 1H), 1.76-1.61 (m, 2H).

### Example A-71: 5-[(3R)-3-[4-(propan-2-yl)benzamido]piperidin-1-yl]-3-[(pyridin-2-yl)amino]pyrazine-2-carboxamide (71)

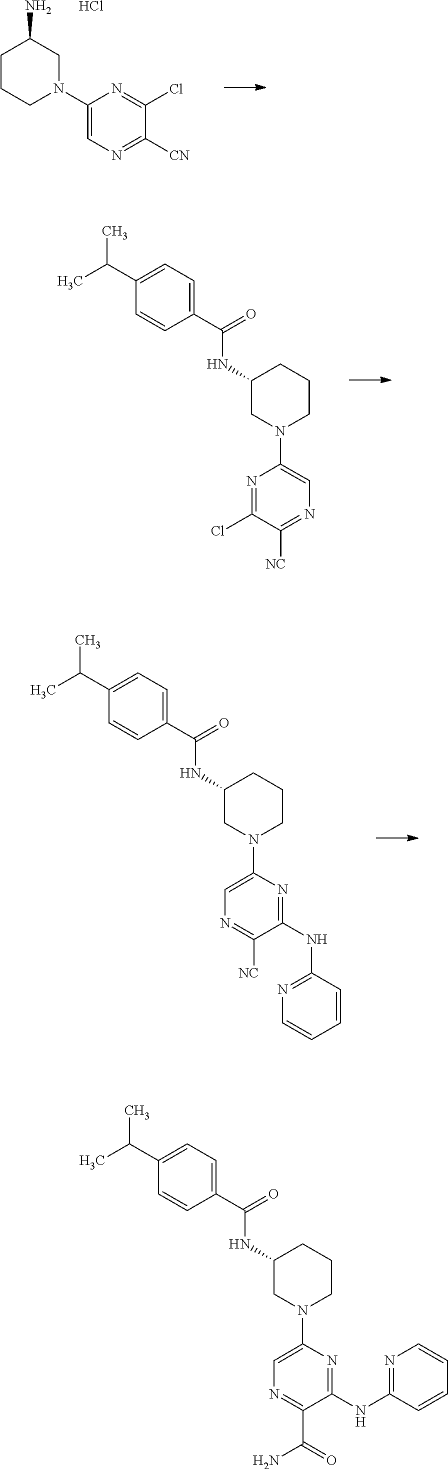 [SMILES: N#Cc1ncc(N2CCC[C@@H](N)C2)nc1Cl ; Cl ; CC(C)c1ccc(C(=O)N[C@@H]2CCCN(c3cnc(C#N)c(Cl)n3)C2)cc1 ; CC(C)c1ccc(C(=O)N[C@@H]2CCCN(c3cnc(C#N)c(Nc4ccccn4)n3)C2)cc1 ; CC(C)c1ccc(C(=O)N[C@@H]2CCCN(c3cnc(C(N)=O)c(Nc4ccccn4)n3)C2)cc1]

In a similar manner as described in Example A-8, N-[(3R)-1-(6-chloro-5-cyanopyrazin-2-yl)piperidin-3-yl]-4-(propan-2-yl)benzamide was prepared using 4-(propan-2-yl)benzoic acid. MS found for C20H22ClN5O as (M+H)⁺ 384.0. To a solution of N-[(3R)-1-(6-chloro-5-cyanopyrazin-2-yl)piperidin-3-yl]-4-(propan-2-yl)benzamide (201.3 mg, 0.52 mmol) in 1,4-dioxane (3 mL), t-BuONa (64.7 mg, 0.67 mmol), pyridin-2-amine (60.2 mg, 0.63 mmol), xantphos (39.3 mg, 0.068 mmol) and Pd₂(dba)₃ (44.9 mg, 0.052 mmol) were added. The suspension was degassed under N₂ then heated to 95° C. and stirred at this temperature for 4 hours. DCM (100 mL) and H₂O (50 mL) were added. The phases were separated, the organic one was concentrated and purified by flash-chromatography (silica), MeOH in DCM from 0 to 10% to give N-[(3R)-1-{5-cyano-6-[(pyridin-2-yl)amino]pyrazin-2-yl}piperidin-3-yl]-4-(propan-2-yl)benzamide (92 mg, 40% yield) as a yellow solid. MS found for C25H27N7O as (M+H)⁺ 442.0. To a suspension of N-[(3R)-1-(5-cyano-6-[(pyridin-2-yl)amino]pyrazin-2-yl)piperidin-3-yl]-4-(propan-2-yl)benzamide (92 mg, 0.21 mmol) in MeOH/DMSO (3/0.2 mL) TEA (0.5 mL, 3.6 mmol), H₂O₂ 30% in water (0.15 mL) and NaOH (22.4 mg, 0.56 mmol) were added. The mixture was stirred at room temperature overnight then it was concentrated and partitioned between DCM and water. The organic layer was concentrated and purified by preparative HPLC to give 5-[(3R)-3-[4-(propan-2-yl)benzamido]piperidin-1-yl]-3-[(pyridin-2-yl)amino]pyrazine-2-carboxamide (44.3 mg, 46% yield) as a yellowish solid. MS found for C28H30N6O2 as (M+H)⁺ 460.2. ¹H NMR (500 MHz, DMSO) δ 11.74 (s, 1H), 8.32 (d, J=7.41 Hz, 1H), 8.29 (d, J=8.51 Hz, 1H), 8.27-8.24 (m, 1H), 7.85-7.82 (m, 1H), 7.81-7.77 (m, 3H), 7.72-7.67 (m, 1H), 7.43 (d, J=2.20 Hz, 1H), 7.34 (d, J=8.23 Hz, 2H), 6.97 (ddd, J=7.27, 4.80, 0.82 Hz, 1H), 4.48 (d, J=11.25 Hz, 1H), 4.20 (d, J=13.17 Hz, 1H), 3.97 (dd, J=6.86, 3.02 Hz, 1H), 3.27-3.17 (m, 1H), 3.15-3.07 (m, 1H), 2.99-2.89 (m, 1H), 2.03-1.95 (m, 1H), 1.94-1.87 (m, 1H), 1.81-1.71 (m, 1H), 1.69-1.56 (m, 1H), 1.22 (d, J=6.86 Hz, 6H).

### Example A-72: N-[(3R)-1-{5-carbamoyl-6-[(3-methyl-1,2-thiazol-5-yl)amino]pyrazin-2-yl}piperidin-3-yl]-1-ethyl-3a,7a-dihydro-1H-indazole-5-carboxamide (72)

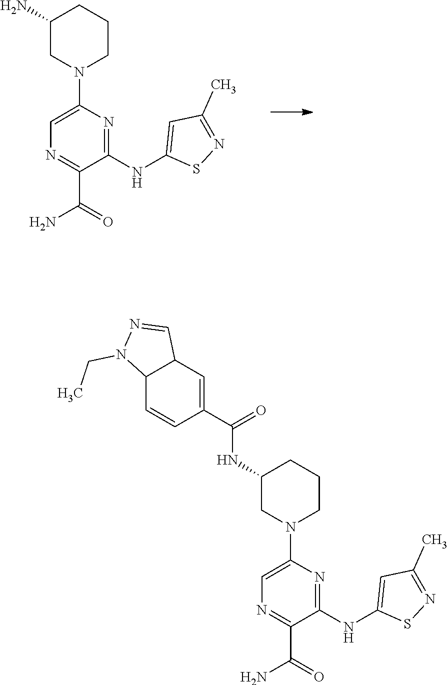 [SMILES: Cc1cc(Nc2nc(N3CCC[C@@H](N)C3)cnc2C(N)=O)sn1 ; CCN1N=CC2C=C(C(=O)N[C@@H]3CCCN(c4cnc(C(N)=O)c(Nc5cc(C)ns5)n4)C3)C=CC21]

In a similar manner as described in Example A-8, N-[(3R)-1-{5-carbamoyl-6-[(3-methyl-1,2-thiazol-5-yl)amino]pyrazin-2-yl}piperidin-3-yl]-1-ethyl-3a,7a-dihydro-1H-indazole-5-carboxamide (72) was prepared using 1-ethyl-1H-indazole-5-carboxylic acid. MS found for C24H29N9O2S as (M+H)⁺ 506.4. ¹H NMR (500 MHz, DMSO) δ 12.29 (s, 1H), 8.46 (d, J=7.41 Hz, 1H), 8.31 (dd, J=1.65, 0.82 Hz, 1H), 8.20 (d, J=0.82 Hz, 1H), 7.97-7.82 (m, 3H), 7.73 (d, J=9.06 Hz, 1H), 7.54 (d, J=1.92 Hz, 1H), 6.85 (s, 1H), 4.47 (q, J=7.23 Hz, 4H), 4.08-3.94 (m, 1H), 3.39-3.22 (m, 2H), 2.27 (s, 3H), 2.09-1.99 (m, 1H), 1.98-1.89 (m, 1H), 1.84-1.72 (m, 1H), 1.69-1.58 (m, 1H), 1.40 (t, J=7.27 Hz, 3H).

### Example A-73: Synthesis of 5-[(3R)-3-(4-cyclopropyl-3-fluorobenzamido)piperidin-1-yl]-3-[(3-methyl-1,2-thiazol-5-yl)amino]pyrazine-2-carboxamide (73)

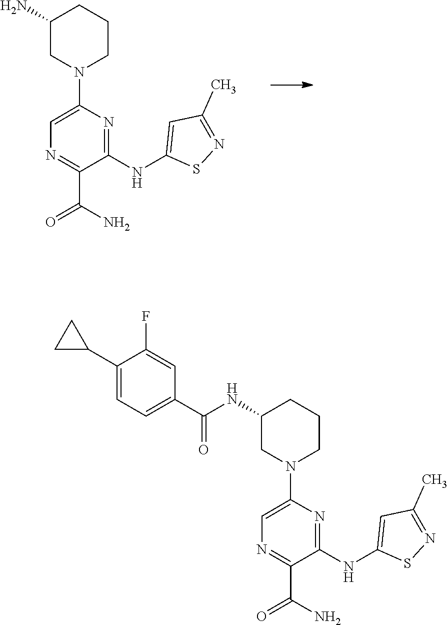 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(C5CC5)c(F)c4)C3)cnc2C(N)=O)sn1]

In a similar manner as described in Example A-8, 5-[(3R)-3-(4-cyclopropyl-3-fluorobenzamido)piperidin-1-yl]-3-[(3-methyl-1,2-thiazol-5-yl)amino]pyrazine-2-carboxamide (73) was prepared using 4-cyclopropyl-3-fluorobenzoic acid. MS found for C24H26FN7O2S as (M+H)⁺ 496.0. ¹H NMR (400 MHz, DMSO) δ 12.28 (s, 1H), 8.44 (d, J=7.28 Hz, 1H), 7.90 (br. s., 1H), 7.83 (s, 1H), 7.63-7.49 (m, 3H), 7.07 (t, J=8.03 Hz, 1H), 6.84 (s, 1H), 4.58-4.31 (m, 2H), 4.06-3.89 (m, 1H), 3.42-3.15 (m, 2H), 2.28 (s, 3H), 2.15-2.04 (m, 1H), 2.04-1.87 (m, 2H), 1.80-1.55 (m, 2H), 1.10-0.75 (m, 4H).

### Example A-74: 5-[(2R,3R)-3-(4-cyclopropyl-2-fluorobenzamido)-2-methylpiperidin-1-yl]-3-[(3-methyl-1,2-thiazol-5-yl)amino]pyrazine-2-carboxamide (74)

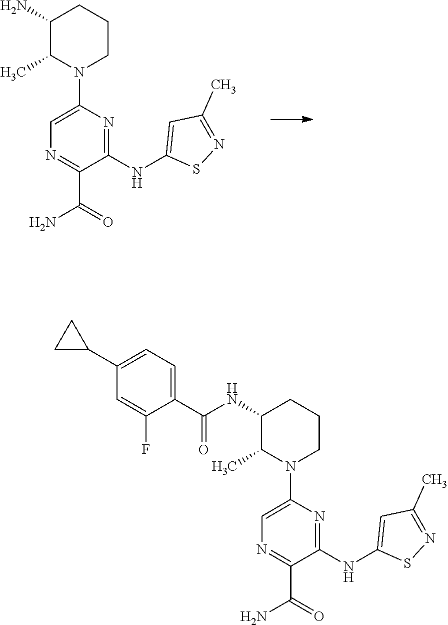 [SMILES: Cc1cc(Nc2nc(N3CCC[C@@H](N)[C@H]3C)cnc2C(N)=O)sn1 ; Cc1cc(Nc2nc(N3CCC[C@@H](NC(=O)c4ccc(C5CC5)cc4F)[C@H]3C)cnc2C(N)=O)sn1]

In a similar manner as described in Example A-8, 5-[(2R,3R)-3-(4-cyclopropyl-2-fluorobenzamido)-2-methylpiperidin-1-yl]-3-[(3-methyl-1,2-thiazol-5-yl)amino]pyrazine-2-carboxamide (74) was prepared using 4-cyclopropyl-2-fluorobenzoic acid. MS found for C25H28FN7O2S as (M+H)⁺ 510.0. ¹H NMR (500 MHz, DMSO) δ 12.28 (s, 1H), 8.31 (d, J=7.55 Hz, 1H), 7.90 (s, 1H), 7.81 (s, 1H), 7.55 (s, 1H), 7.45 (t, J=7.80 Hz, 1H), 7.05-6.96 (m, 2H), 6.83 (s, 1H), 5.12 (br. s., 1H), 4.39 (br. s., 1H), 4.15-3.96 (m, 1H), 3.20-3.06 (m, 1H), 2.27 (s, 3H), 2.07-1.95 (m, 1H), 1.95-1.79 (m, 2H), 1.74-1.53 (m, 2H), 1.22 (d, J=6.72 Hz, 3H), 1.07-0.97 (m, 2H), 0.83-0.71 (m, 2H).

### Example A-75: Synthesis of 5-(4-{[(dimethylcarbamoyl)amino]methyl}piperidin-1-yl)-3-[(3-methyl-1,2-thiazol-5-yl)amino]pyrazine-2-carboxamide (75)

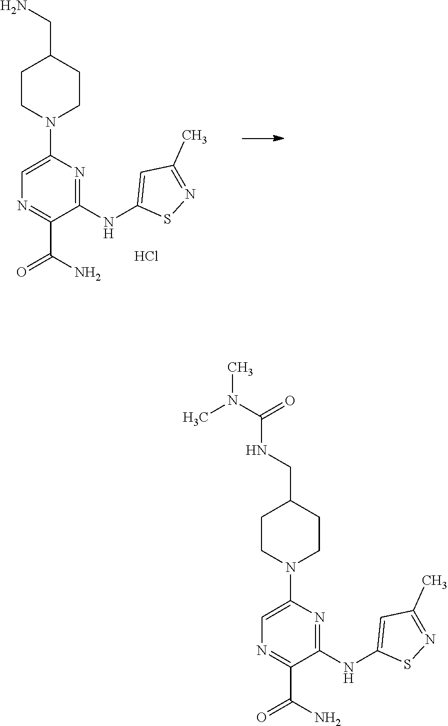 [SMILES: Cc1cc(Nc2nc(N3CCC(CN)CC3)cnc2C(N)=O)sn1 ; Cl ; Cc1cc(Nc2nc(N3CCC(CNC(=O)N(C)C)CC3)cnc2C(N)=O)sn1]

In a similar manner as described in Example A-7, 5-(4-{([(dimethylcarbamoyl)amino]methyl}piperidin-1-yl)-3-[(3-methyl-1,2-thiazol-5-yl)amino]pyrazine-2-carboxamide (75) was prepared using dimethylcarbamyl chloride. MS found for C18H26N8O2S as (M+H)⁺ 419.0. ¹H NMR (400 MHz, DMSO) δ 12.30 (br. s, 1H), 7.91-7.84 (m, 1H), 7.84-7.80 (m, 1H), 7.58-7.49 (m, 1H), 6.89-6.82 (m, 1H), 6.33 (br. s., 1H), 4.64-4.47 (m, 2H), 3.11 (t, J=11.98 Hz, 2H), 3.00-2.88 (m, 2H), 2.78 (s, 6H), 2.30 (s, 3H), 1.89-1.72 (m, 3H), 1.25-1.07 (m, 2H).

### Example A-76: Synthesis of 3-[(3-methyl-1,2-thiazol-5-yl)amino]-5-{4-[(phenylformamido)methyl]piperidin-1-yl}pyrazine-2-carboxamide (76)

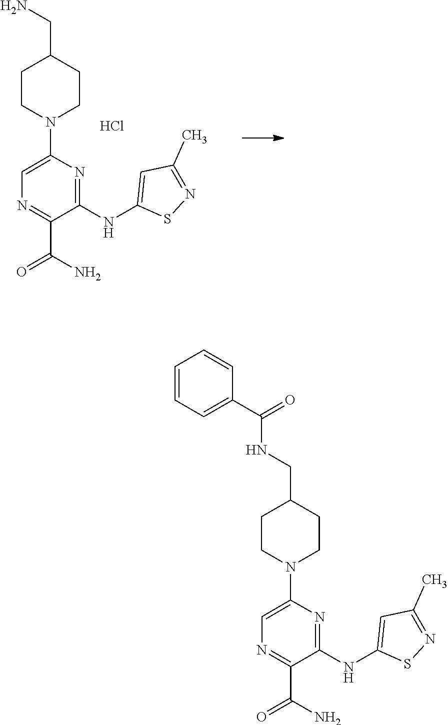 [SMILES: Cc1cc(Nc2nc(N3CCC(CN)CC3)cnc2C(N)=O)sn1 ; Cl ; Cc1cc(Nc2nc(N3CCC(CNC(=O)c4ccccc4)CC3)cnc2C(N)=O)sn1]

In a similar manner as described in Example A-7, 3-[(3-methyl-1,2-thiazol-5-yl)amino]-5-{4-[(phenylformamido)methyl]piperidin-1-yl}pyrazine-2-carboxamide (76) was prepared using benzoyl chloride. MS found for C22H25N7O2S as (M+H)⁺ 452.15. ¹H NMR (400 MHz, DMSO) δ 12.29 (s, 1H) 8.54 (m, J=4.89 Hz, 1H), 7.90-7.81 (m, 4H), 7.56-7.49 (m, 2H), 7.46 (m, J=7.83 Hz, 2H), 6.85 (s, 1H), 4.69-4.48 (m, 2H), 3.20 (t, J=6.36 Hz, 2H), 3.18-3.07 (m, 2H), 2.29 (s, 3H), 2.05-1.92 (m, 1H), 1.87 (d, J=11.74 Hz, 2H), 1.33-1.20 (m, 2H).

### Example A-77: 5-[4-({[4-(dimethylamino)phenyl]formamido}methyl)piperidin-1-yl]-3-[(3-methyl-1,2-thiazol-5-yl)amino]pyrazine-2-carboxamide (77)

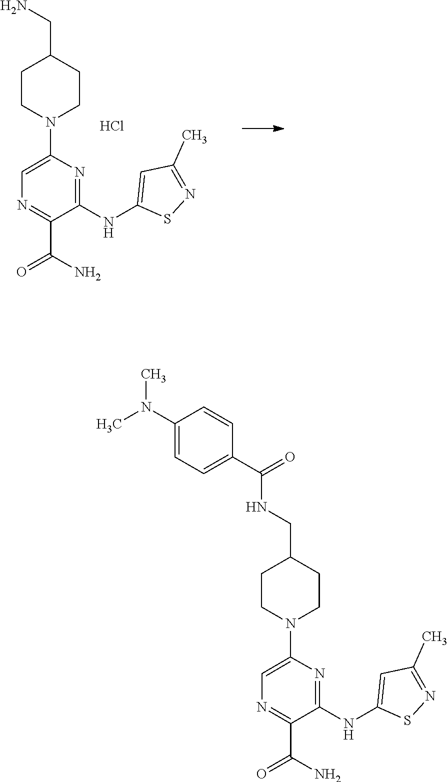 [SMILES: Cc1cc(Nc2nc(N3CCC(CN)CC3)cnc2C(N)=O)sn1 ; Cl ; Cc1cc(Nc2nc(N3CCC(CNC(=O)c4ccc(N(C)C)cc4)CC3)cnc2C(N)=O)sn1]

In a similar manner as described in Example A-7, 5-[4-({[4-(dimethylamino)phenyl]formamido}methyl)piperidin-1-yl]-3-[(3-methyl-1,2-thiazol-5-yl)amino]pyrazine-2-carboxamide (77) was prepared using 4-(dimethylamino)benzoyl chloride. MS found for C24H30N8O2S as (M+H)⁺ 495.2. ¹H NMR (400 MHz, DMSO) δ 12.27 (br. s, 1H), 8.18 (t, J=5.87 Hz, 1H), 7.87 (br. s., 1H), 7.83 (s, 1H), 7.73 (d, J=8.80 Hz, 2H), 7.52 (br. s., 1H), 6.85 (s, 1H), 6.69 (d, J=9.29 Hz, 2H), 4.64-4.49 (m, 2H), 3.21-3.06 (m, 4H), 2.96 (s, 6H), 2.29 (s, 3H), 2.04-1.89 (m, 1H), 1.84 (d, J=10.76 Hz, 2H), 1.30-1.18 (m, 2H).

### Example A-78: 5-(3-({[(dimethylcarbamoyl)amino]methyl}piperidin-1-yl)-3-[(3-methyl-1,2-thiazol-5-yl)amino]pyrazine-2-carboxamide (78)

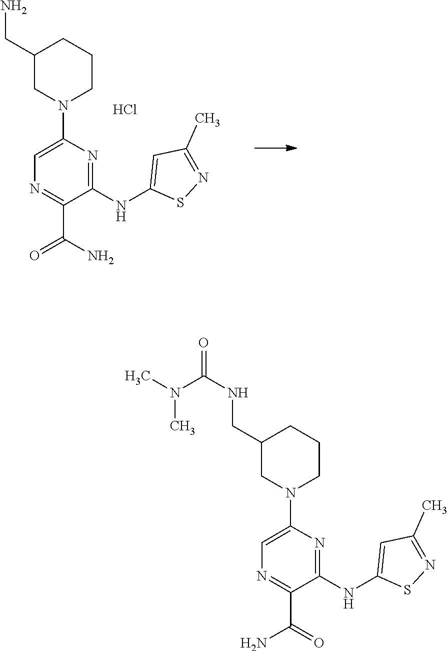 [SMILES: Cc1cc(Nc2nc(N3CCCC(CN)C3)cnc2C(N)=O)sn1 ; Cl ; Cc1cc(Nc2nc(N3CCCC(CNC(=O)N(C)C)C3)cnc2C(N)=O)sn1]

In a similar manner as described in Example A-47, 5-(3-{[(dimethylcarbamoyl)amino]methyl}piperidin-1-yl)-3-[(3-methyl-1,2-thiazol-5-yl)amino]pyrazine-2-carboxamide (78) was prepared using dimethylcarbamyl chloride. MS found for C18H26N8O2S as (M+H)⁺ 419.0. ¹H NMR (400 MHz, DMSO) δ 12.27 (s, 1H), 7.84 (br. s., 1H), 7.75 (s, 1H), 7.49 (br. s., 1H), 6.81 (s, 1H), 6.30 (t, J=5.48 Hz, 1H), 4.47-4.25 (m, 2H), 3.20 (t, J=11.29 Hz, 1H), 3.07-2.88 (m, 3H), 2.75 (s, 6H), 2.27 (s, 3H), 1.86-1.67 (m, 3H), 1.55-1.19 (m, 2H).

### Example A-79: 3-[(3-methyl-1,2-thiazol-5-yl)amino]-5-{3-[(phenylformamido)methyl]piperidin-1-yl}pyrazine-2-carboxamide (79)

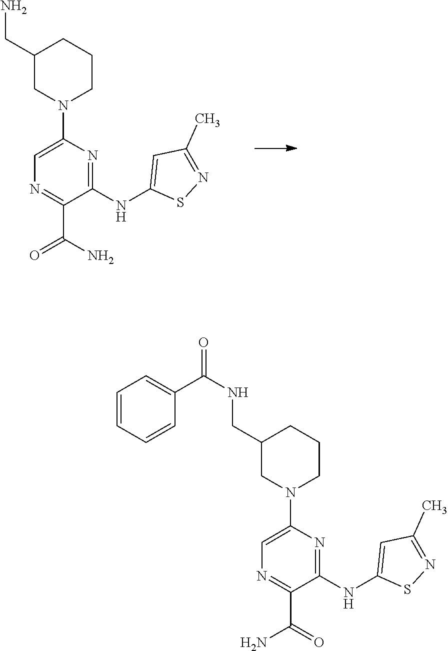 [SMILES: Cc1cc(Nc2nc(N3CCCC(CN)C3)cnc2C(N)=O)sn1 ; Cc1cc(Nc2nc(N3CCCC(CNC(=O)c4ccccc4)C3)cnc2C(N)=O)sn1]

In a similar manner as described in Example A-47, 3-[(3-methyl-1,2-thiazol-5-yl)amino]-5-{3-[(phenylformamido)methyl]piperidin-1-yl}pyrazine-2-carboxamide (79) was prepared using benzoyl chloride. MS found for C22H25N7O2S as (M+H)⁺ 452.0. ¹H NMR (400 MHz, DMSO) δ 12.28 (s, 1H), 8.54 (t, J=5.76 Hz, 1H), 7.89-7.86 (m, 1H), 7.86-7.82 (m, 2H), 7.79 (s, 1H), 7.54-7.48 (m, 2H), 7.44 (m, J=7.68 Hz, 2H), 6.83 (s, 1H), 4.52-4.31 (m, 2H), 3.34-3.24 (m, 3H), 3.12 (dd, J=13.45, 9.88 Hz, 1H), 2.29 (s, 3H), 1.98-1.79 (m, 3H), 1.61-1.47 (m, 1H), 1.46-1.35 (m, 1H).

### Example A-80: Synthesis of 5-[3-({[4-dimethylamino)phenyl]formamido}methyl)piperidin-1-yl]-3-[(3-methyl-1,2-thiazol-1-yl)amino]pyrazine-2-carboxamide (80)

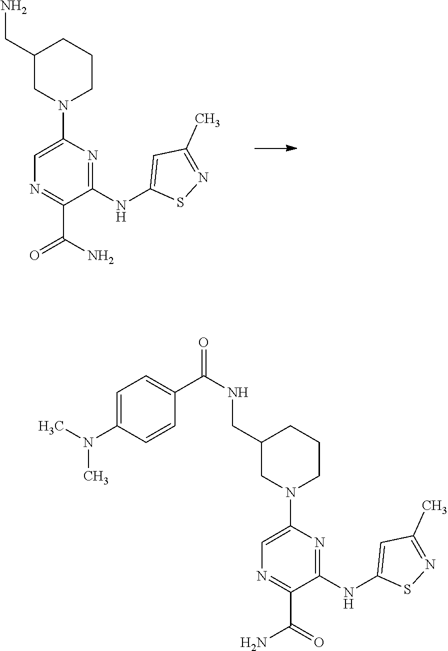 [SMILES: Cc1cc(Nc2nc(N3CCCC(CN)C3)cnc2C(N)=O)sn1 ; Cc1cc(Nc2nc(N3CCCC(CNC(=O)c4ccc(N(C)C)cc4)C3)cnc2C(N)=O)sn1]

In a similar manner as described in Example A-47, 5-[3-({[4-dimethylamino)phenyl]formamido}methyl)piperidin-1-yl]-3-[(3-methyl-1,2-thiazol-5-yl)amino]pyrazine-2-carboxamide (80) was prepared using 4-(dimethylamino) benzoyl chloride. MS found for C24H30N8O2S as (M+H)⁺ 495.0. ¹H NMR (400 MHz, DMSO) δ 12.26 (s, 1H), 8.16 (t, J=5.59 Hz, 1H), 7.85 (br. s., 1H), 7.74 (s, 1H), 7.70 (d, J=8.77 Hz, 2H), 7.49 (br. s., 1H), 6.81 (s, 1H), 6.66 (d, J=8.99 Hz, 2H), 4.47-4.27 (m, 2H), 3.27-3.16 (m, 3H), 3.07 (dd, J=13.15, 10.09 Hz, 1H), 2.94 (s, 6H), 2.27 (s, 3H), 1.93-1.73 (m, 3H), 1.57-1.28 (m, 2H).

### Example A-81: 5-[(2R,3R)-3-(4-cyclopropyl-2-fluorobenzamido)-2-methylpiperidin-1-yl]-3-{[4-(4-methylpiperazin-1-yl)phenyl]amino}pyrazine-2-carboxamide (81)

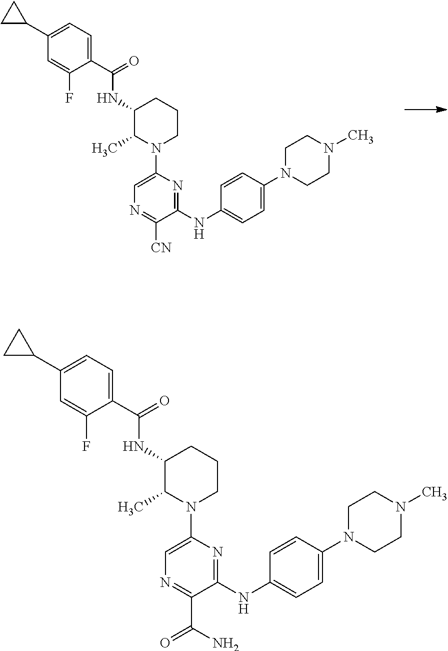 [SMILES: C[C@@H]1[C@H](NC(=O)c2ccc(C3CC3)cc2F)CCCN1c1cnc(C#N)c(Nc2ccc(N3CCN(C)CC3)cc2)n1 ; C[C@@H]1[C@H](NC(=O)c2ccc(C3CC3)cc2F)CCCN1c1cnc(C(N)=O)c(Nc2ccc(N3CCN(C)CC3)cc2)n1]

In a similar manner as described in Example A-40, 5-[(2R,3R)-3-(4-cyclopropyl-2-fluorobenzamido)-2-methylpiperidin-1-yl]-3-{[4-(4-methylpiperazin-1-yl)phenyl]amino}pyrazine-2-carboxamide (81) was prepared using NaOH and H₂O₂. MS found for C32H39FN8O2 as (M+H)⁺ 487.0. ¹H NMR (400 MHz, DMSO) δ 10.94 (s, 1H), 8.31 (d, J=7.41 Hz, 1H), 7.74-7.67 (m, 1H), 7.59 (s, 1H), 7.48 (t, J=7.82 Hz, 1H), 7.44 (d, J=9.06 Hz, 2H), 7.28 (d, J=2.20 Hz, 1H), 7.04-6.97 (m, 2H), 6.80 (d, J=9.06 Hz, 2H), 5.26-4.99 (m, 1H), 4.21-3.94 (m, 2H), 3.10-2.99 (m, 1H), 2.96-2.83 (m, 4H), 2.42-2.29 (m, 4H), 2.20 (s, 3H), 2.07-1.96 (m, 1H), 1.89-1.50 (m, 4H), 1.10 (d, J=6.86 Hz, 3H), 1.07-1.00 (m, 2H), 0.81-0.73 (m, 2H).

### Example A-82: N-[(3R)-1-[5-cyano-6-({4-[(dimethylamino)methyl]phenyl}amino)pyrazin-2-yl]piperidin-3-yl]-4-(propan-2-yl)benzamide (82)

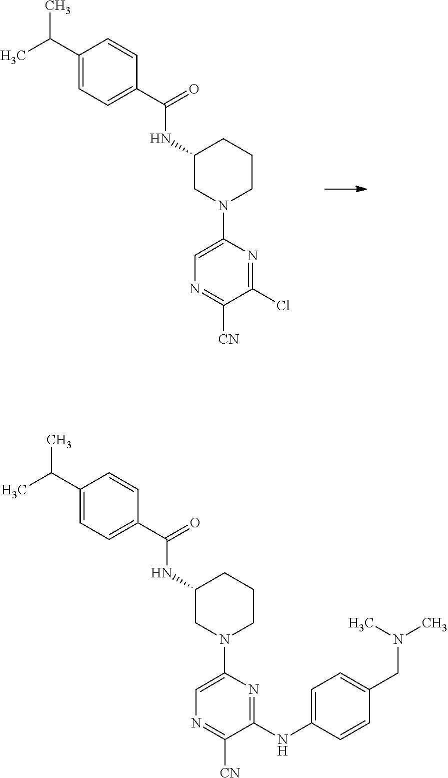 [SMILES: CC(C)c1ccc(C(=O)N[C@@H]2CCCN(c3cnc(C#N)c(Cl)n3)C2)cc1 ; CC(C)c1ccc(C(=O)N[C@@H]2CCCN(c3cnc(C#N)c(Nc4ccc(CN(C)C)cc4)n3)C2)cc1]

To a solution of N-[(3R)-1-(6-chloro-5-cyanopyrazin-2-yl)piperidin-3-yl]-4-(propan-2-yl)benzamide (194.5 mg, 0.51 mmol) in 1,4-dioxane (5 mL) Cs₂CO₃ (681.3 mg, 2.09 mmol), 4-[(dimethylamino)methyl]aniline hydrochloride (119.3 mg, 0.79 mmol), BINAP (66.7 mg, 0.11 mmol) and Pd(AcO)₂ (22.4 mg, 0.1 mmol) were added. The mixture was stirred at 90° C. for 5 hours then it was partitioned between DCM and water. The combined organic phases were concentrated and purified by preparative HPLC to give N-[(3R)-1-[5-cyano-6-({(4-[(dimethylamino)methyl]phenyl}amino)pyrazin-2-yl]piperidin-3-yl]-4-(propan-2-yl)benzamide (62.2 mg, 24% yield) as a yellow solid. MS found for C₂₉H₃₅N₇O as (M+H)⁺ 498.5. ¹H NMR (400 MHz, DMSO) δ 8.99 (s, 1H), 8.29 (d, J=7.96 Hz, 1H), 7.86-7.82 (m, 1H), 7.79 (d, J=8.23 Hz, 2H), 7.47 (d, J=8.51 Hz, 2H), 7.34 (d, J=8.23 Hz, 2H), 7.12 (d, J=8.23 Hz, 2H), 4.48-4.06 (m, 2H), 3.99-3.88 (m, 1H), 3.20 (br. s., 2H), 3.17-3.09 (m, 1H), 3.07-2.87 (m, 2H), 2.02 (s, 6H), 1.97-1.91 (m, 1H), 1.88-1.77 (m, 1H), 1.76-1.63 (m, 1H), 1.62-1.47 (m, 1H), 1.22 (d, J=6.86 Hz, 6H).

### Example A-83: 3-({4-[(dimethylamino)methyl]phenyl}amino)-5-[(3R)-3-[4-(propan-2-yl)benzamido]piperidin-1-yl]pyrazine-2-carboxamide (83)

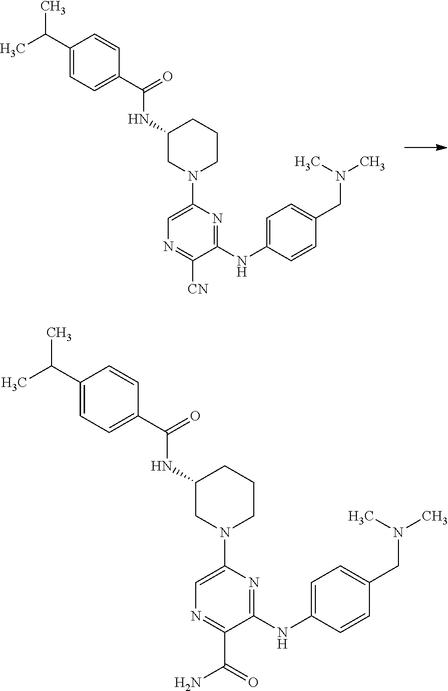

In similar manner as described in Example A-40, 3-({4-[(dimethylamino)methyl]phenyl}amino)-5-[(3R)-3-[4-(propan-2-yl)benzamido]piperidin-1-yl]pyrazine-2-carboxamide (83) was prepared using NaOH and H₂O₂. MS found for C29H37N7O2 as (M+H)⁺ 516.3. ¹H NMR (400 MHz, DMSO) δ 11.26 (s, 1H), 8.32 (d, J=7.67 Hz, 1H), 7.86-7.72 (m, 3H), 7.69 (s, 1H), 7.54 (d, J=8.40 Hz, 2H), 7.38-7.26 (m, 3H), 7.15 (d, J=8.40 Hz, 2H), 4.44 (d, J=10.41 Hz, 1H), 4.19 (d, J=12.72 Hz, 1H), 4.04-3.90 (m, 1H), 3.24 (s, 2H), 3.21-3.10 (m, 1H), 3.09-3.00 (m, 1H), 2.99-2.89 (m, 1H), 2.07 (s, 6H), 2.03-1.82 (m, 2H), 1.81-1.51 (m, 2H), 1.22 (d, J=6.91 Hz, 6H).

### Example A-84: (R)-5-(3-acrylamidopiperidin-1-yl)-3-(4-(1-cyclopentylpiperidin-4-yl)phenylamino)pyrazine-2-carboxamide (84)

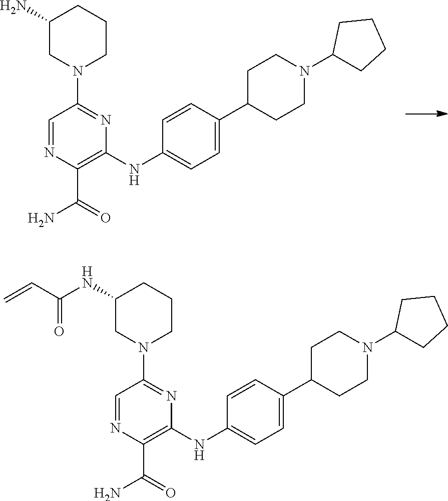 [SMILES: NC(=O)c1ncc(N2CCC[C@@H](N)C2)nc1Nc1ccc(C2CCN(C3CCCC3)CC2)cc1 ; C=CC(=O)N[C@@H]1CCCN(c2cnc(C(N)=O)c(Nc3ccc(C4CCN(C5CCCC5)CC4)cc3)n2)C1]

(R)-5-(3-aminopiperidin-1-yl)-3-(4-(1-cyclopentylpiperidin-4-yl)phenylamino)pyrazine-2-carboxamide, described in Example A-1, (HCl salt, 15 mg, 0.032 mmol) was dissolved in 2 mL NMP. To it was added DIPEA (28 μL, 0.16 mmol), and the mixture was stirred in ice bath. To it was added acryloyl chloride (5.3 μL, 0.064 mmol). After 10 min, the reaction was quenched with 100 μL TFA, and the mixture was subjected to reverse phase preparative HPLC (mobile phases: 0.1% formic acid in water and neat acetonitrile) to isolate the title compound (R)-5-(3-acrylamidopiperidin-1-yl)-3-(4-(1-cyclopentylpiperidin-4-yl)phenylamino)pyrazine-2-carboxamide as formic acid salt (9.8 mg). MS found for C29H39N7O2 as (M+H)⁺ 518.7, and (M−H)⁻ 516.3.

### Example A-85: 5-((2R,3R)-3-acrylamido-2-methylpiperidin-1-yl)-3-(4-(1-cyclopentylpiperidin-4-yl)phenylamino)pyrazine-2-carboxamide (85)

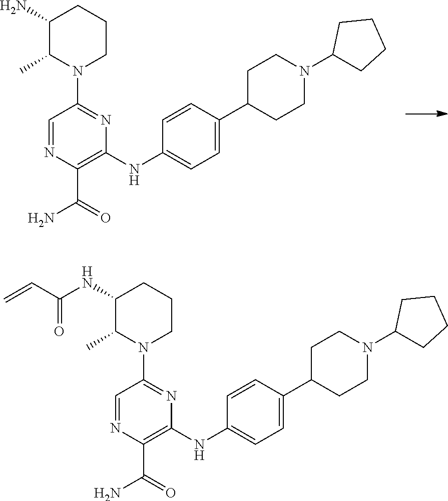 [SMILES: C[C@@H]1[C@H](N)CCCN1c1cnc(C(N)=O)c(Nc2ccc(C3CCN(C4CCCC4)CC3)cc2)n1 ; C=CC(=O)N[C@@H]1CCCN(c2cnc(C(N)=O)c(Nc3ccc(C4CCN(C5CCCC5)CC4)cc3)n2)[C@@H]1C]

In a similar manner described in Example A-84, the title compound 5-((2R,3R)-3-acrylamido-2-methylpiperidin-1-yl)-3-(4-(1-cyclopentylpiperidin-4-yl)phenylamino)pyrazine-2-carboxamide was prepared using 5-((2R,3R)-3-amino-2-methylpiperidin-1-yl)-3-(4-(1-cyclopentylpiperidin-4-yl)phenylamino)pyrazine-2-carboxamide, which had been prepared by the same synthetic scheme described in Example A-54 for 5-[(2S,3S)-3-amino-2-methylpiperidin-1-yl]-3-[(3-methyl-1,2-thiazol-5-yl)amino]pyrazine-2-carboxamide. MS found for C30H41N7O2 as (M+H)⁺ 532.5, and (M−H)⁻ 530.4.

### Example A-86: (R)-5-(3-acrylamidopiperidin-1-yl)-3-(3-methylisothiazol-5-ylamino)pyrazine-2-carboxamide (86)

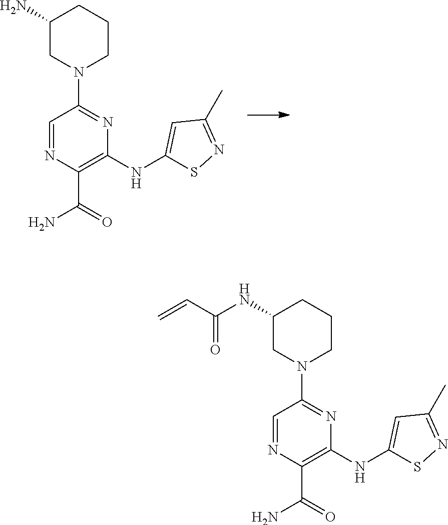 [SMILES: Cc1cc(Nc2nc(N3CCC[C@@H](N)C3)cnc2C(N)=O)sn1 ; C=CC(=O)N[C@@H]1CCCN(c2cnc(C(N)=O)c(Nc3cc(C)ns3)n2)C1]

In a similar manner described in Example A-84, the title compound (R)-5-(3-acrylamidopiperidin-1-yl)-3-(3-methylisothiazol-5-ylamino)pyrazine-2-carboxamide was prepared using (R)-5-(3-aminopiperidin-1-yl)-3-(3-methylisothiazol-5-ylamino)pyrazine-2-carboxamide, which had been prepared by the same synthetic scheme described in Example A-1 for (R)-5-(3-aminopiperidin-1-yl)-3-(4-(1-cyclopentylpiperidin-4-yl)phenylamino)pyrazine-2-carboxamide with 3-methyl-5-aminoisothiazole hydrochloride to replace 4-(1-cyclopentylpiperidin-4-yl)aniline. MS found for C17H21N7O2S as (M+H)⁺ 388.1, and (M−H)⁻ 386.1.

### Example A-87: (S)-5-(3-acrylamidopiperidin-1-yl)-3-(3-methylisothiazol-5-ylamino)pyrazine-2-carboxamide (87)

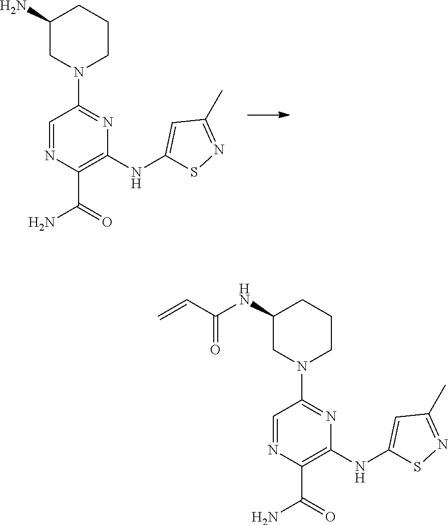 [SMILES: Cc1cc(Nc2nc(N3CCC[C@H](N)C3)cnc2C(N)=O)sn1 ; C=CC(=O)N[C@H]1CCCN(c2cnc(C(N)=O)c(Nc3cc(C)ns3)n2)C1]

In a similar manner described in Example A-86, the title compound (S)-5-(3-acrylamidopiperidin-1-yl)-3-(3-methylisothiazol-5-ylamino)pyrazine-2-carboxamide was prepared using (S)-5-(3-aminopiperidin-1-yl)-3-(3-methylisothiazol-5-ylamino)pyrazine-2-carboxamide. MS found for C17H21N7O2S as (M+H)⁺ 388.2, and (M−H)⁻ 386.1.

### Example A-88: (R)-5-(3-acrylamidopiperidin-1-yl)-3-(3-(pyrimidin-2-yl)phenylamino)pyrazine-2-carboxamide (88)

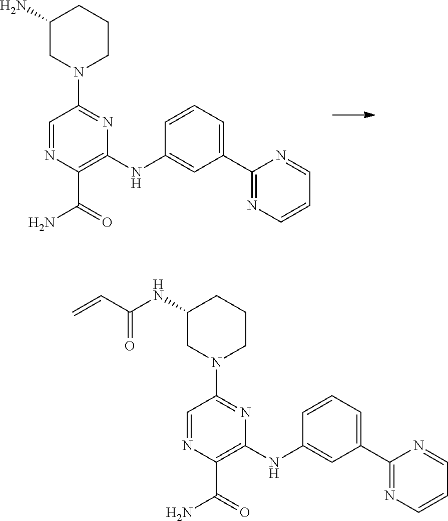 [SMILES: NC(=O)c1ncc(N2CCC[C@@H](N)C2)nc1Nc1cccc(-c2ncccn2)c1 ; C=CC(=O)N[C@@H]1CCCN(c2cnc(C(N)=O)c(Nc3cccc(-c4ncccn4)c3)n2)C1]

In a similar manner described in Example A-84, the title compound (R)-5-(3-acrylamidopiperidin-1-yl)-3-(3-(pyrimidin-2-yl)phenylamino)pyrazine-2-carboxamide was prepared using (R)-5-(3-aminopiperidin-1-yl)-3-(3-(pyrimidin-2-yl)phenylamino)pyrazine-2-carboxamide, which had been prepared by the same synthetic scheme described in Example A-1 for (R)-5-(3-aminopiperidin-1-yl)-3-(4-(1-cyclopentylpiperidin-4-yl)phenylamino)pyrazine-2-carboxamide with commercial 3-(pyrimidin-2-yl)aniline. MS found for C23H24N8O2 as (M+H)⁺ 445.3, and (M−H)⁻ 443.1.

### Example A-89: (S)-5-(3-acrylamidopiperidin-1-yl)-3-(4-(pyrimidin-2-yl)phenylamino)pyrazine-2-carboxamide (89)

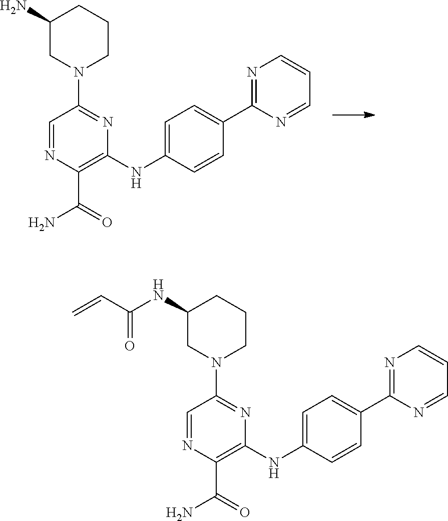 [SMILES: NC(=O)c1ncc(N2CCC[C@H](N)C2)nc1Nc1ccc(-c2ncccn2)cc1 ; C=CC(=O)N[C@H]1CCCN(c2cnc(C(N)=O)c(Nc3ccc(-c4ncccn4)cc3)n2)C1]

In a similar manner described in Example A-84, the title compound (S)-5-(3-acrylamidopiperidin-1-yl)-3-(4-(pyrimidin-2-yl)phenylamino)pyrazine-2-carboxamide was prepared using (S)-5-(3-aminopiperidin-1-yl)-3-(4-(pyrimidin-2-yl)phenylamino)pyrazine-2-carboxamide, which had been prepared by the same synthetic scheme described in Example A-I for (R)-5-(3-aminopiperidin-1-yl)-3-(4-(1-cyclopentylpiperidin-4-yl)phenylamino)pyrazine-2-carboxamide with commercial 4-(pyrimidin-2-yl)aniline and (S)-(3-BOC-amino)piperidine. MS found for C23H24N8O2 as (M+H)⁺ 445.3, and (M−H)⁻ 443.2.

### Example A-90: Synthesis of 3-(4-acryloyl-1,4-diazepan-1-yl)-5-(4-isopropylphenylamino)-1,2,4-triazine-6-carboxamide (90)

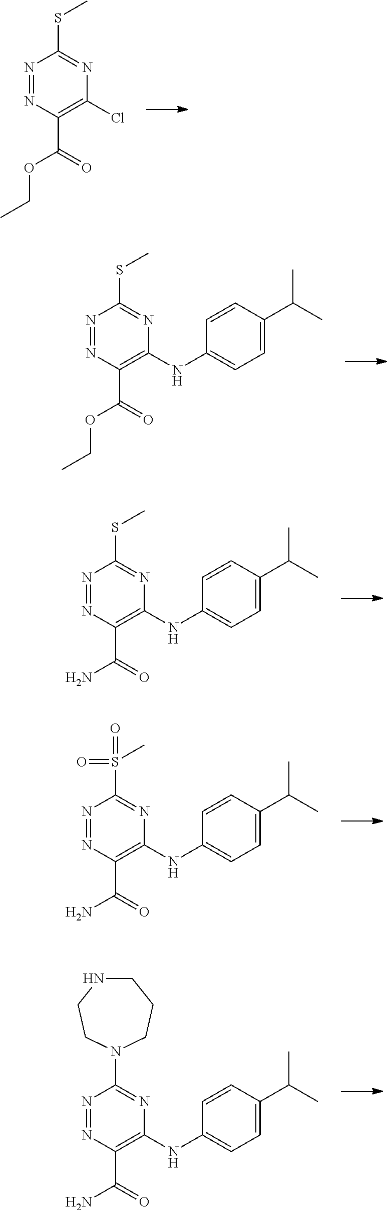 [SMILES: CCOC(=O)c1nnc(SC)nc1Cl ; CCOC(=O)c1nnc(SC)nc1Nc1ccc(C(C)C)cc1 ; CSc1nnc(C(N)=O)c(Nc2ccc(C(C)C)cc2)n1 ; CC(C)c1ccc(Nc2nc(S(C)(=O)=O)nnc2C(N)=O)cc1 ; CC(C)c1ccc(Nc2nc(N3CCCNCC3)nnc2C(N)=O)cc1]

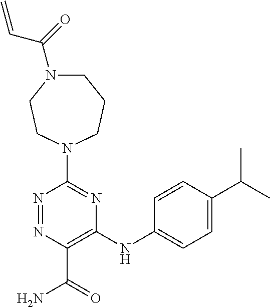 [SMILES: C=CC(=O)N1CCCN(c2nnc(C(N)=O)c(Nc3ccc(C(C)C)cc3)n2)CC1]

To a light yellow solution of commercially available ethyl 5-chloro-3-(methylthio)-1,2,4-triazine-6-carboxylate (3.00 g, 12.88 mmol) was added 4-isopropylaniline (2.2 mL, 15.45 mmol), resulting in gelatinous yellow slurry. The mixture was treated dropwise with Hunig's base (2.7 mL, 15.45 mmol), resulting in a clear brown solution. After 5 minutes of stirring, LCMS confirmed clean transformation into the SnAr product, ethyl 5-(4-isopropylphenylamino)-3-(methylthio)-1,2,4-triazine-6-carboxylate: MH⁺=333.3.

To the reaction mixture was added 7 N ammonia in methanol (74 mL, 515 mmol). Within 30 minutes of stirring, pale yellow solid starts crushing out of the solution. Stirred for a total of 4 hours, upon which time the pale yellow solid was isolated through a disposable ChemGlass filter funnel (cat# OP-6602-12), washed with cold acetonitrile (2×20 mL) and cold hexanes (2×25 mL), then air-vacuum dried for 1 hr to isolate 3.35 g 5-(4-isopropylphenylamino)-3-(methylthio)-1,2,4-triazine-6-carboxamide 3.35 g, (86% yield): MH⁺=304.0.

To a yellow solution of 5-(4-isopropylphenylamino)-3-(methylthio)-1,2,4-triazine-6-carboxamide (3.35 g, 11.04 mmol) in 245 ml THF, under the atmosphere of nitrogen, was added dry mCPBA (7.42 g, 33.13 mmol) in small portions. The resulting solution became yellow slurry within 1 hr. At 2 hr time point LCMS showed progressing oxidation with a 1:5 ratio of sulfoxide/sulfone products. The mixture was stirred for additional 2 hrs, to allow maximum product precipitation, then was filtered through a disposable ChemGlass filter funnel (cat# OP-6602-12), washed with cold DCM (4×15 ml), air-vacuum dried for overnight to produce 5-(4-isopropylphenylamino)-3-(methylsulfonyl)-1,2,4-triazine-6-carboxamide (2.91 g, 78%) as yellow powder; MW=335.4, MH⁺=336.0.

A yellow solution of 5-(4-isopropylphenylamino)-3-(methylsulfonyl)-1,2,4-triazine-6-carboxamide (50 mg, 2.24 mmol), 1-BOC-homopiperazine (60 mg, 0.30 mmol) and DIPEA (50 μL, 0.30 mmol) in 3 mL NMP was heated at 90° C. for 2 hours, then cooled to 70° C. To the mixture was added 2 mL TFA, and it was stirred at 70° C. for 1 hour. Then the mixture was cooled to RT and directly subjected to reverse phase preparative HPLC to isolate 3-(1,4-diazepan-1-yl)-5-(4-isopropylphenylamino)-1,2,4-triazine-6-carboxamide as HCl salt (60 mg). MS found for C18H25N7O as (M+H)⁺ 356.3.

To a solution of 3-(1,4-diazepan-1-yl)-5-(4-isopropylphenylamino)-1,2,4-triazine-6-carboxamide (HCl salt, 20 mg, 0.051 mmol) in 1.7 mL NMP was added DIPEA (63 μL, 0.36 mmol) and 2 minutes later acryloyl chloride (8.3 μL, 0.10 mmol). The mixture was stirred for 10 minutes, then quenched with 0.2 mL TFA and diluted with 3 ml water. The crude was purified directly by reverse phase preparative HPLC, using 0.1% formic acid in water and acetonitrile as mobile phase, to give the title compound 3-(4-acryloyl-1,4-diazepan-1-yl)-5-(4-isopropylphenylamino)-1,2,4-triazine-6-carboxamide (12.2 mg, 58%) as formic acid salt. MS found for C21H27N7O2 as (M+H)⁺ 445.3, and (M−H)⁻ 443.2.

### Example A-91: 3-(4-carbamoylpiperidin-1-yl)-5-(4-isopropylphenylamino)-1,2,4-triazine-6-carboxamide

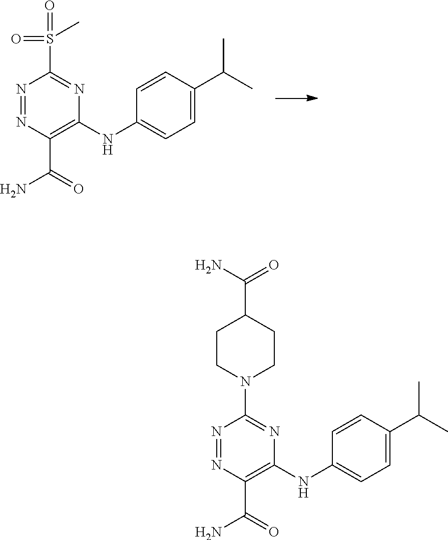 [SMILES: CC(C)c1ccc(Nc2nc(S(C)(=O)=O)nnc2C(N)=O)cc1 ; CC(C)c1ccc(Nc2nc(N3CCC(C(N)=O)CC3)nnc2C(N)=O)cc1]

In a similar manner described in Example A-90, the title compound 3-(4-carbamoylpiperidin-1-yl)-5-(4-isopropylphenylamino)-1,2,4-triazine-6-carboxamide was prepared using piperidine-4-carboxamide. MS found for C19H25N7O2 as (M+H)⁺ 384.3, and (M−H)⁻ 382.2.

The degree of inhibition of a panel of kinases is determined using the in vitro HotSpot kinase assay (purified enzymes, ³³P-ATP, an appropriate substrate and 1 μM ATP).

Compounds in Table 1 were prepared using procedures similar to those described herein or known in the art.

TABLE 1
Additional Compounds of Formula (A-I)

| Cmpd. | | |
|---|---|---|
| No. | Structure | Name |
| A-94 | 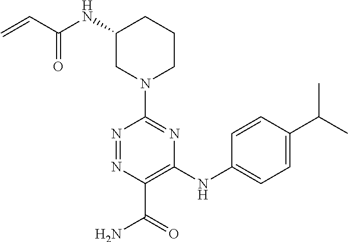 [SMILES: C=CC(=O)N[C@@H]1CCCN(c2nnc(C(N)=O)c(Nc3ccc(C(C)C)cc3)n2)C1] | (R)-3-(3-acrylamido- piperidin-1-yl)-5-(4- isopropylphenylamino)-1,2,4- triazine-6-carboxamide MS: M + H = 410.3; M − H = 408.1 |
| A-95 | 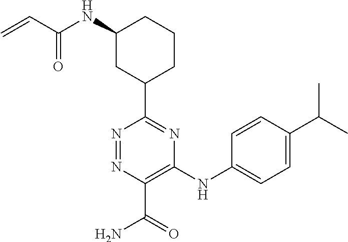 [SMILES: C=CC(=O)N[C@H]1CCCC(c2nnc(C(N)=O)c(Nc3ccc(C(C)C)cc3)n2)C1] | (S)-3-(3-acrylamido- piperidin-1-yl)-5-(4- isopropylphenylamino)-1,2,4- triazine-6-carboxamide MS: M + H = 410.4; M − H = 408.1 |
| A-96 | 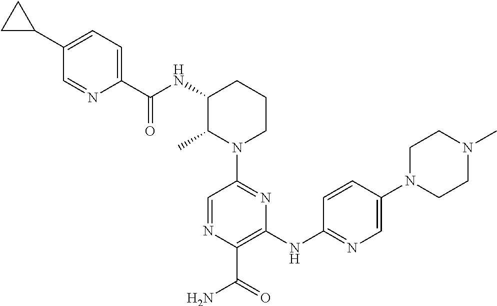 [SMILES: C[C@@H]1[C@H](NC(=O)c2ccc(C3CC3)cn2)CCCN1c1cnc(C(N)=O)c(Nc2ccc(N3CCN(C)CC3)cn2)n1] | 5-((2R,3R)-3-(5- cyclopropylpicolinamido)-2- methylpiperidin-1-yl)-3-(5-(4- methylpiperazin-1-yl)pyridin-2- ylamino)pyrazine-2- carboxamide |
| A-98 | 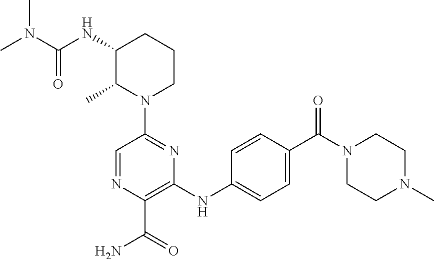 | 5-[(2R,3R)-3- [(dimethylcarbamoyl)amino]-2- methylpiperidin-1-yl]-3-{[4-(4- methylpiperazine-1- carbonyl)phenyl]amino}pyrazine- 2-carboxamide |
| A-99 | 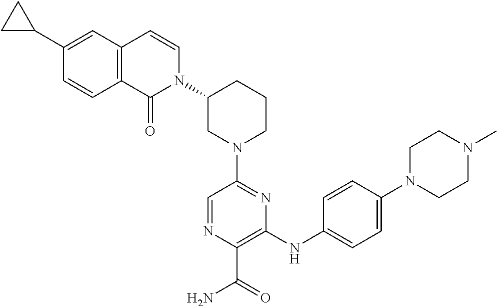 [SMILES: CN1CCN(c2ccc(Nc3nc(N4CCC[C@@H](n5ccc6cc(C7CC7)ccc6c5=O)C4)cnc3C(N)=O)cc2)CC1] | 5-[(3R)-3-(6-cyclo- propyl-1-oxo- 1,2-dihydroisoquinolin-2- yl)piperidin-1-yl]-3-{[4-(4- methylpiperazin-1- yl)phenyl]amino}pyrazine-2- carboxamide |
| A-100 | 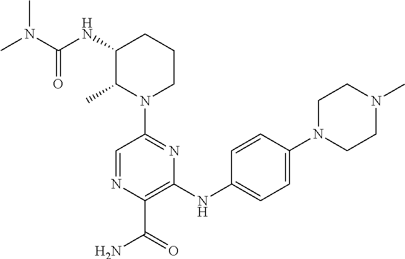 | 5-[(2R,3R)-3- [(dimethylcarbamoyl)amino]-2- methylpiperidin-1-yl]-3-{[4-(4- methylpiperazin-1- yl)phenyl]amino}pyrazine-2- carboxamide |
| A-101 | 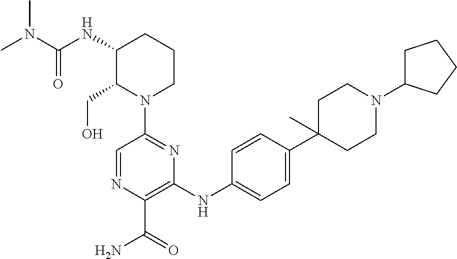 [SMILES: CN(C)C(=O)N[C@@H]1CCCN(c2cnc(C(N)=O)c(Nc3ccc(C4(C)CCN(C5CCCC5)CC4)cc3)n2)[C@@H]1CO] | 3-{[4-(1-cyclopentyl-4- methylpiperidin-4- yl)phenyl]amino}-5-[(2S,3R)-3- [(dimethylcarbamoyl)amino]-2- (hydroxymethyl)piperidin-1- yl]pyrazine-2-carboxamide |
| A-102 | 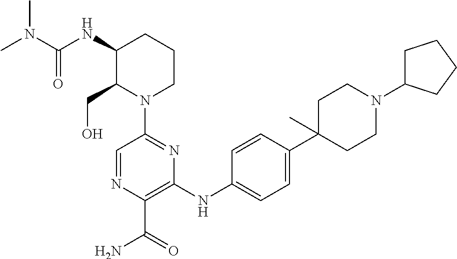 [SMILES: CN(C)C(=O)N[C@H]1CCCN(c2cnc(C(N)=O)c(Nc3ccc(C4(C)CCN(C5CCCC5)CC4)cc3)n2)[C@H]1CO] | 3-{[4-(1-cyclopentyl-4- methylpiperidin-4- yl)phenyl]amino}-5-[(2R,3S)-3- [(dimethylcarbamoyl)amino]-2- (hydroxymethyl)piperidin-1- yl]pyrazine-2-carboxamide |
| A-103 | 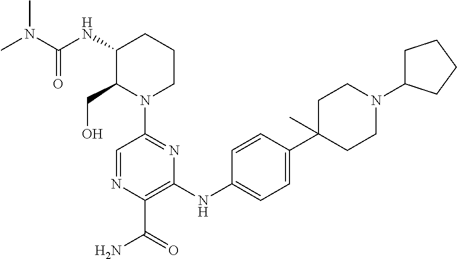 [SMILES: CN(C)C(=O)N[C@@H]1CCCN(c2cnc(C(N)=O)c(Nc3ccc(C4(C)CCN(C5CCCC5)CC4)cc3)n2)[C@H]1CO] | 3-{[4-(1-cyclopentyl-4- methylpiperidin-4- yl)phenyl]amino}-5-[(2R,3R)-3- [(dimethylcarbamoyl)amino]-2- (hydroxymethyl)piperidin-1- yl]pyrazine-2-carboxamide |
| A-104 | 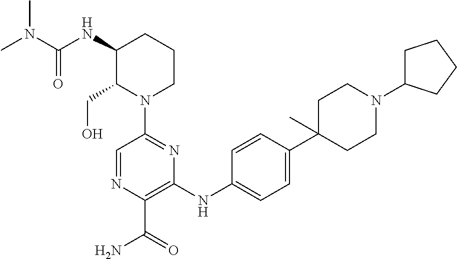 [SMILES: CN(C)C(=O)N[C@H]1CCCN(c2cnc(C(N)=O)c(Nc3ccc(C4(C)CCN(C5CCCC5)CC4)cc3)n2)[C@@H]1CO] | 3-{[4-(1-cyclopentyl-4- methylpiperidin-4- yl)phenyl]amino}-5-[(2S,3S)-3- [(dimethylcarbamoyl)amino]-2- (hydroxymethyl)piperidin-1- yl]pyrazine-2-carboxamide |
| A-105 | 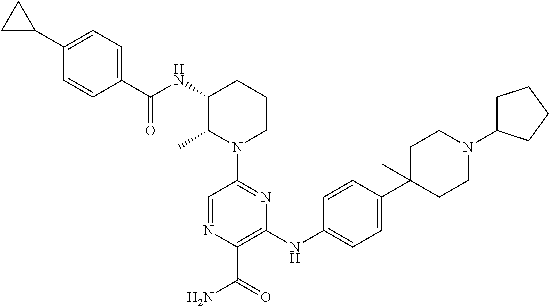 [SMILES: C[C@@H]1[C@H](NC(=O)c2ccc(C3CC3)cc2)CCCN1c1cnc(C(N)=O)c(Nc2ccc(C3(C)CCN(C4CCCC4)CC3)cc2)n1] | 3-{[4-(1-cyclopentyl-4- methylpiperidin-4- yl)phenyl]amino}- 5-[(2R,3R)-3-(4- cyclopropylbenzamido)-2- methylpiperidin-1-yl]pyrazine- 2-carboxamide |
| A-106 | 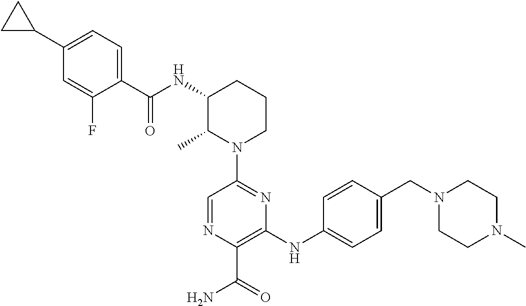 [SMILES: C[C@@H]1[C@H](NC(=O)c2ccc(C3CC3)cc2F)CCCN1c1cnc(C(N)=O)c(Nc2ccc(CN3CCN(C)CC3)cc2)n1] | 5-[(2R,3R)-3-(4-cyclopropyl-2- fluorobenzamido)-2- methylpiperidin-1-yl]-3-({4-[(4- methylpiperazin-1-yl)meth- yl]phenyl}amino)pyrazine- 2-carboxamide |
| A-107 | 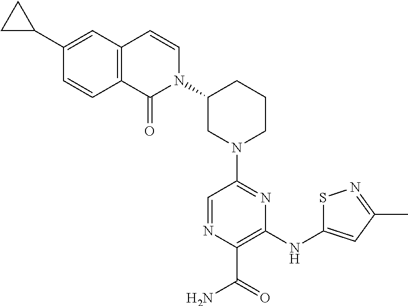 [SMILES: Cc1cc(Nc2nc(N3CCC[C@@H](n4ccc5cc(C6CC6)ccc5c4=O)C3)cnc2C(N)=O)sn1] | 5-[(3R)-3-(6-cyclopropyl-1-oxo- 1,2-dihydroisoquinolin-2- yl)piperidin-1-yl]-3- [(3-methyl-1,2- thiazol-5-yl)amino]pyrazine-2- carboxamide |
| A-108 | 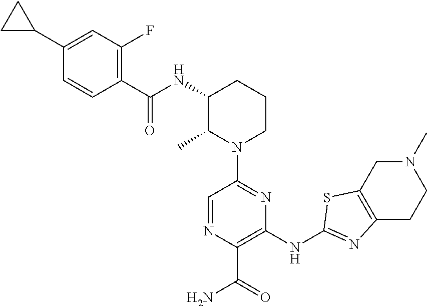 [SMILES: C[C@@H]1[C@H](NC(=O)c2ccc(C3CC3)cc2F)CCCN1c1cnc(C(N)=O)c(Nc2nc3c(s2)CN(C)CC3)n1] | 5-[(2R,3R)-3-(4-cyclopropyl-2- fluorobenzamido)-2- methylpiperidin-1-yl]-3-({5- methyl-4H,5H,6H,7H- [1,3]thiazolo[5,4-c]pyridin-2- yl}amino)pyrazine-2- carboxamide |
| A-109 | 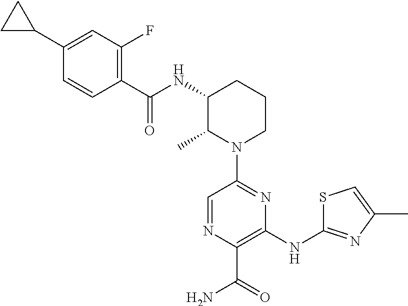 [SMILES: Cc1csc(Nc2nc(N3CCC[C@@H](NC(=O)c4ccc(C5CC5)cc4F)[C@H]3C)cnc2C(N)=O)n1] | 5-[(2R,3R)-3-(4-cyclopropyl-2- fluorobenzamido)-2- methylpiperidin-1-yl]-3-[(4- methyl-1,3-thiazol-2- yl)amino]pyrazine-2- carboxamide |
| A-110 | 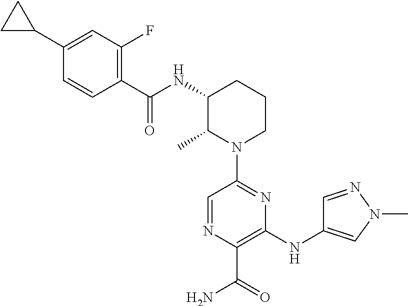 [SMILES: C[C@@H]1[C@H](NC(=O)c2ccc(C3CC3)cc2F)CCCN1c1cnc(C(N)=O)c(Nc2cnn(C)c2)n1] | 5-[(2R,3R)-3-(4-cyclopropyl-2- fluorobenzamido)-2- methylpiperidin-1-yl]-3-[(1- methyl-1H-pyrazol-4- yl)amino]pyrazine-2- carboxamide |
| A-111 | 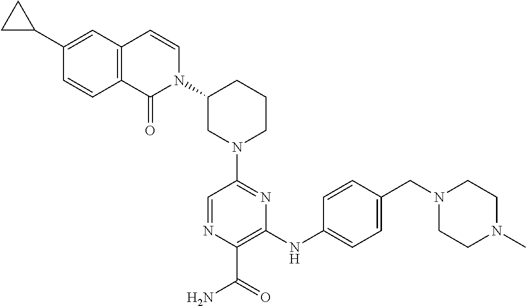 [SMILES: CN1CCN(Cc2ccc(Nc3nc(N4CCC[C@@H](n5ccc6cc(C7CC7)ccc6c5=O)C4)cnc3C(N)=O)cc2)CC1] | 5-[(3R)-3-(6-cyclopropyl-1- oxo-1,2-dihydroisoquinolin-2- yl)piperidin-1-yl]-3-({4-[(4- methylpiperazin-1-yl)meth- yl]phenyl}amino)pyrazine- 2-carboxamide |
| A-112 | 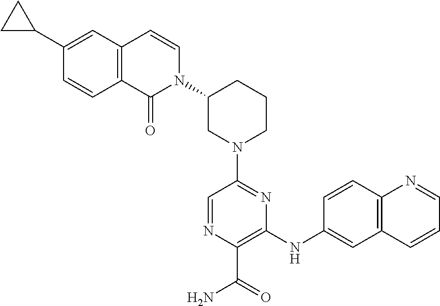 [SMILES: NC(=O)c1ncc(N2CCC[C@@H](n3ccc4cc(C5CC5)ccc4c3=O)C2)nc1Nc1ccc2ncccc2c1] | 5-[(3R)-3-(6-cyclopropyl-1- oxo-1,2-dihydroisoquinolin-2- yl)piperidin-1-yl]-3- [(quinolin-6- yl)amino]pyrazine-2- carboxamide |
| A-113 | 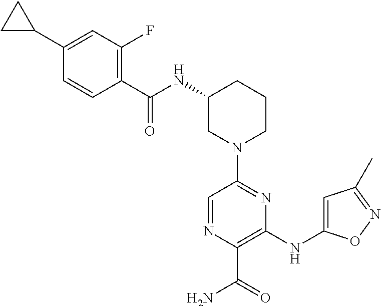 [SMILES: Cc1cc(Nc2nc(N3CCC[C@@H](NC(=O)c4ccc(C5CC5)cc4F)C3)cnc2C(N)=O)on1] | 5-[(3R)-3-(4-cyclopropyl-2- fluorobenzamido)piperidin-1-yl]- 3-[(3-methyl-1,2-oxazol-5- yl)amino]pyrazine-2- carboxamide |
| A-114 | 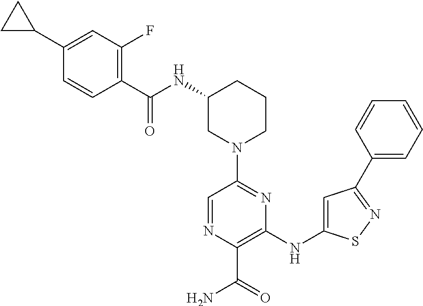 [SMILES: NC(=O)c1ncc(N2CCC[C@@H](NC(=O)c3ccc(C4CC4)cc3F)C2)nc1Nc1cc(-c2ccccc2)ns1] | 5-[(3R)-3-(4-cyclopropyl-2- fluorobenzamido)piperidin-1-yl]-3- [(3-phenyl-1,2-thiazol-5- yl)amino]pyrazine-2- carboxamide |
| A-115 | 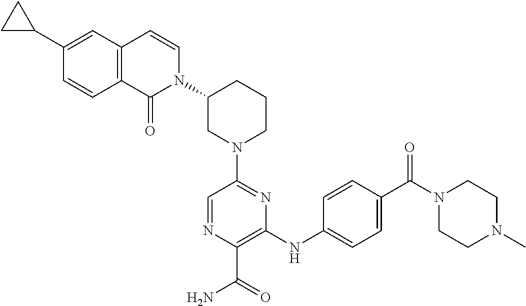 [SMILES: CN1CCN(C(=O)c2ccc(Nc3nc(N4CCC[C@@H](n5ccc6cc(C7CC7)ccc6c5=O)C4)cnc3C(N)=O)cc2)CC1] | 5-[(3R)-3-(6-cyclo- propyl-1-oxo- 1,2-dihydroisoquinolin-2- yl)piperidin-1-yl]-3-{[4-(4- methylpiperazine-1- carbonyl)phenyl]amino}pyr- azine-2-carboxamide |
| A-116 | 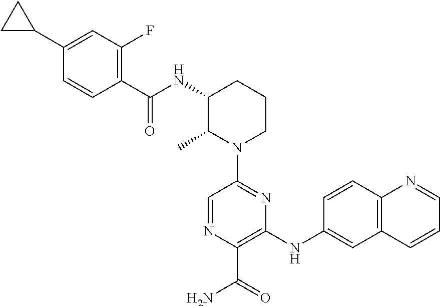 [SMILES: C[C@@H]1[C@H](NC(=O)c2ccc(C3CC3)cc2F)CCCN1c1cnc(C(N)=O)c(Nc2ccc3ncccc3c2)n1] | 5-[(2R,3R)-3-(4-cyclopropyl- 2-fluorobenzamido)-2- methylpiperidin- 1-yl]-3-[(quinolin- 6-yl)amino]pyrazine-2- carboxamide |
| A-117 | 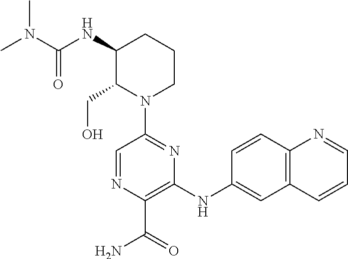 [SMILES: CN(C)C(=O)N[C@H]1CCCN(c2cnc(C(N)=O)c(Nc3ccc4ncccc4c3)n2)[C@@H]1CO] | 5-[(2S,3S)-3- [(dimethyl- carbamoyl)amino]-2- (hydroxymethyl)piperidin- 1-yl]-3-[(quinolin-6- yl)amino]pyrazine-2- carboxamide |
| A-118 | 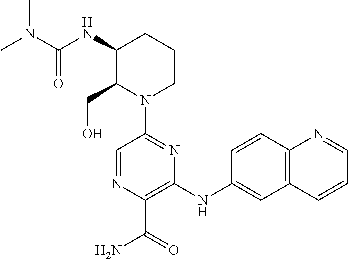 [SMILES: CN(C)C(=O)N[C@H]1CCCN(c2cnc(C(N)=O)c(Nc3ccc4ncccc4c3)n2)[C@H]1CO] | 5-[(2R,3S)-3- [(dimethyl- carbamoyl)amino]-2- (hydroxymethyl)piperidin- 1-yl]-3-[(quinolin- 6-yl)amino]pyrazine- 2-carboxamide |
| A-119 | 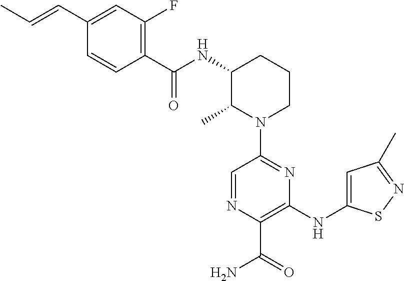 [SMILES: C/C=C/c1ccc(C(=O)N[C@@H]2CCCN(c3cnc(C(N)=O)c(Nc4cc(C)ns4)n3)[C@@H]2C)c(F)c1] | 5-[(2R,3R)-3-{2- fluoro-4-[(1E)- prop-1-en-1-yl]benzamido}-2- methylpiperidin-1-yl]-3-[(3- methyl-1,2-thiazol-5- yl)amino]pyridine-2- carboxamide |
| A-120 | 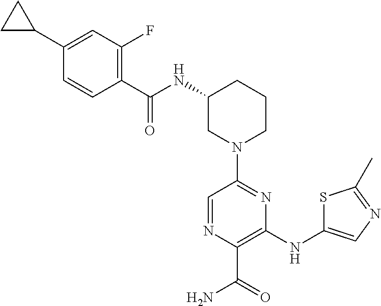 [SMILES: Cc1ncc(Nc2nc(N3CCC[C@@H](NC(=O)c4ccc(C5CC5)cc4F)C3)cnc2C(N)=O)s1] | 5-[(3R)-3-(4-cyclopropyl-2- fluorobenzamido)piperidin- 1-yl]-3- [(2-methyl-1,3-thiazol-5- yl)amino]pyrazine-2- carboxamide |
| A-121 | 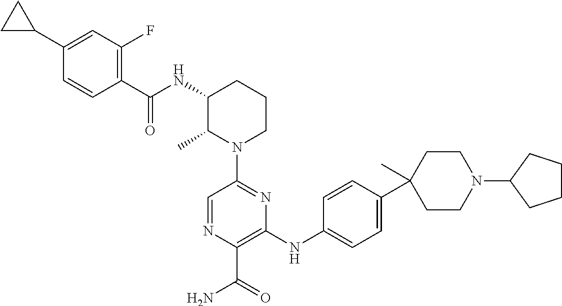 [SMILES: C[C@@H]1[C@H](NC(=O)c2ccc(C3CC3)cc2F)CCCN1c1cnc(C(N)=O)c(Nc2ccc(C3(C)CCN(C4CCCC4)CC3)cc2)n1] | 3-{[4-(1-cyclopentyl-4- methylpiperidin-4- yl)phenyl]amino}-5- [(2R,3R)-3-(4-cyclopropyl- 2-fluorobenzamido)-2- methylpiperidin-1-yl]pyrazine- 2-carboxamide |
| A-122 | 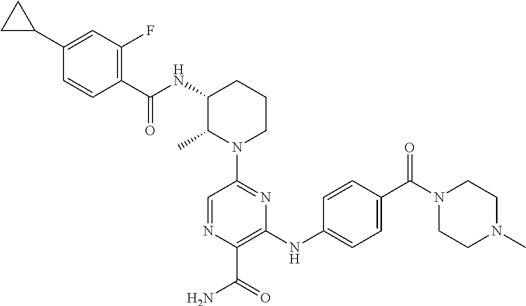 [SMILES: C[C@@H]1[C@H](NC(=O)c2ccc(C3CC3)cc2F)CCCN1c1cnc(C(N)=O)c(Nc2ccc(C(=O)N3CCN(C)CC3)cc2)n1] | 5-[(2R,3R)-3-(4-cyclopropyl- 2-fluorobenzamido)-2- methylpiperidin-1-yl]-3-{[4-(4- methylpiperazine-1- carbonyl)phenyl]amino}pyr- azine-2-carboxamide |
| A-123 | 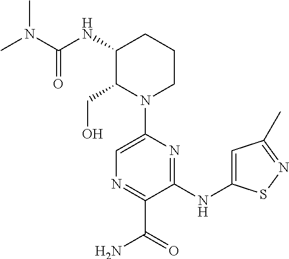 [SMILES: Cc1cc(Nc2nc(N3CCC[C@@H](NC(=O)N(C)C)[C@H]3CO)cnc2C(N)=O)sn1] | 5-[(2S,3R)-3- [(dimethylcarbamoyl)amino]-2- (hydroxymeth- yl)piperidin-1-yl]- 3-[(3-methyl-1,2-thiazol-5- yl)amino]pyrazine-2- carboxamide |
| A-124 | 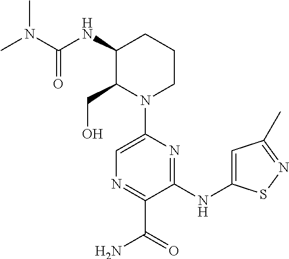 [SMILES: Cc1cc(Nc2nc(N3CCC[C@H](NC(=O)N(C)C)[C@@H]3CO)cnc2C(N)=O)sn1] | 5-[(2R,3S)-3- [(dimethyl- carbamoyl)amino]-2- (hydroxymethyl)piperidin-1-yl]- 3-[(3-methyl-1,2-thiazol-5- yl)amino]pyrazine-2- carboxamide |
| A-125 | 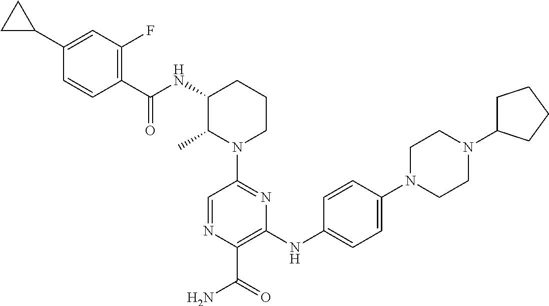 [SMILES: C[C@@H]1[C@H](NC(=O)c2ccc(C3CC3)cc2F)CCCN1c1cnc(C(N)=O)c(Nc2ccc(N3CCN(C4CCCC4)CC3)cc2)n1] | 3-{[4-(4-cyclopentylpiperazin- 1-yl)phenyl]amino}- 5-[(2R,3R)-3-(4-cyclopropyl- 2-fluorobenzamido)-2- methylpiperidin-1-yl]pyrazine- 2-carboxamide |
| A-126 | 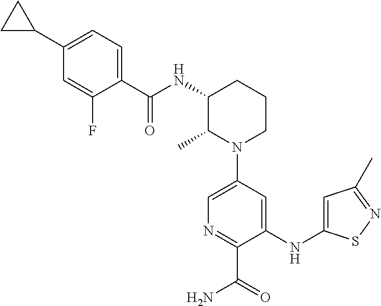 [SMILES: Cc1cc(Nc2cc(N3CCC[C@@H](NC(=O)c4ccc(C5CC5)cc4F)[C@H]3C)cnc2C(N)=O)sn1] | 5-[(2R,3R)-3-(4-cyclopropyl-2- fluorobenzamido)-2- methylpiperidin-1-yl]-3-[(3- methyl-1,2-thiazol-5- yl)amino]pyridine-2- carboxamide |
| A-127 | 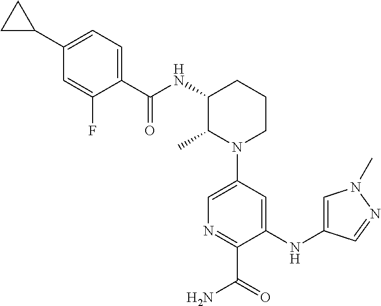 [SMILES: C[C@@H]1[C@H](NC(=O)c2ccc(C3CC3)cc2F)CCCN1c1cnc(C(N)=O)c(Nc2cnn(C)c2)c1] | 5-[(2R,3R)-3-(4-cyclopropyl-2- fluorobenzamido)-2- methylpiperidin-1-yl]-3-[(1- methyl-1H-pyrazol-4- yl)amino]pyridine-2- carboxamide |
| A-128 | 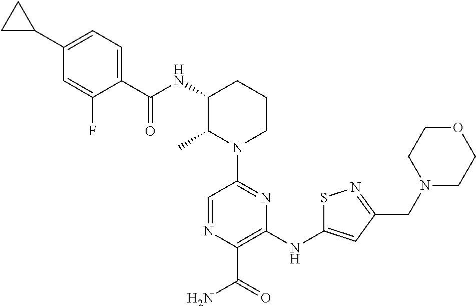 [SMILES: C[C@@H]1[C@H](NC(=O)c2ccc(C3CC3)cc2F)CCCN1c1cnc(C(N)=O)c(Nc2cc(CN3CCOCC3)ns2)n1] | 5-[(2R,3R)-3-(4-cyclopropyl-2- fluorobenzamido)-2- methylpiperidin-1-yl]-3-{[3- (morpholin-4-ylmethyl)-1,2- thiazol-5-yl]amino}pyrazine-2- carboxamide |
| A-129 | 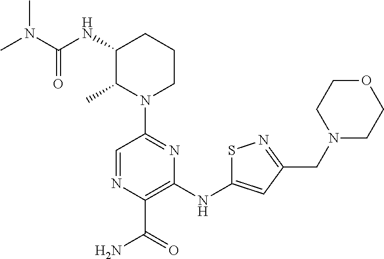 [SMILES: C[C@@H]1[C@H](NC(=O)N(C)C)CCCN1c1cnc(C(N)=O)c(Nc2cc(CN3CCOCC3)ns2)n1] | 5-[(2R,3R)-3- [(dimethylcarbamoyl)amino]-2- methylpiperidin-1-yl]-3-{[3- (morpholin-4-ylmethyl)-1,2- thiazol-5-yl]amino}pyrazine-2- carboxamide |
| A-130 | 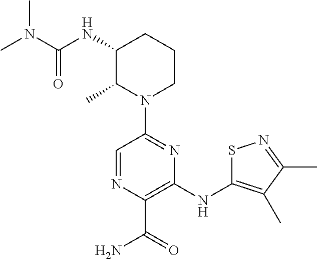 | 3-[(dimethyl-1,2-thiazol-5- yl)amino]-5-[(2R,3R)-3- [(dimethylcarbamoyl)amino]-2- methylpiperidin-1-yl]pyrazine- 2-carboxamide |
| A-131 | 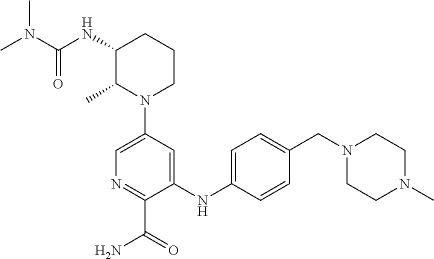 | 5-[(2R,3R)-3- [(dimethylcarbamoyl)amino]-2- methylpiperidin-1-yl]-3-({4-[(4- methylpiperazin-1-yl)meth- yl]phenyl}amino)pyridine- 2-carboxamide |
| A-132 | 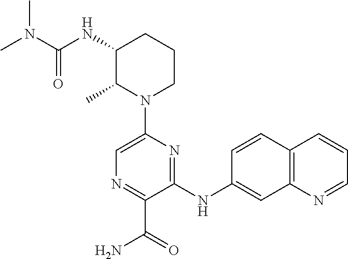 [SMILES: C[C@@H]1[C@H](NC(=O)N(C)C)CCCN1c1cnc(C(N)=O)c(Nc2ccc3cccnc3c2)n1] | 5-[(2R,3R)-3- [(dimethylcarbamoyl)amino]-2- methylpiperidin-1- yl]-3-[(quinolin- 7-yl)amino]pyrazine-2- carboxamide |
| A-133 | 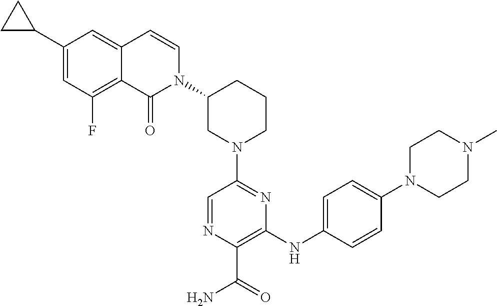 [SMILES: CN1CCN(c2ccc(Nc3nc(N4CCC[C@@H](n5ccc6cc(C7CC7)cc(F)c6c5=O)C4)cnc3C(N)=O)cc2)CC1] | 5-[(3R)-3-(6-cyclo- propyl-8-fluoro- 1-oxo-1,2-dihydroisoquinolin- 2-yl)piperidin-1-yl]-3-{[4-(4- methylpiperazin-1- yl)phenyl]amino}pyrazine-2- carboxamide |
| A-134 | 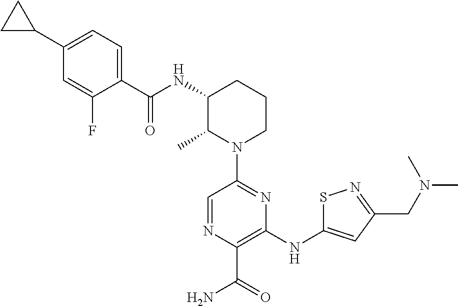 [SMILES: C[C@@H]1[C@H](NC(=O)c2ccc(C3CC3)cc2F)CCCN1c1cnc(C(N)=O)c(Nc2cc(CN(C)C)ns2)n1] | 5-[(2R,3R)-3-(4-cyclopropyl-2- fluorobenzamido)-2- methylpiperidin-1-yl]-3-({3- [(dimethylamino)methyl]-1,2- thiazol-5-yl}amino)pyrazine-2- carboxamide |
| A-135 | 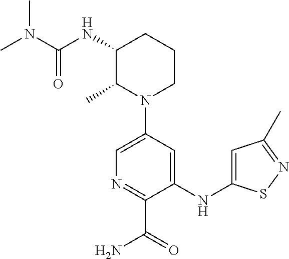 [SMILES: Cc1cc(Nc2cc(N3CCC[C@@H](NC(=O)N(C)C)[C@H]3C)cnc2C(N)=O)sn1] | 5-[(2R,3R)-3- [(dimethylcarbamoyl)amino]-2- methylpiperidin-1-yl]-3-[(3- methyl-1,2-thiazol-5- yl)amino]pyridine-2- carboxamide |
| A-136 | 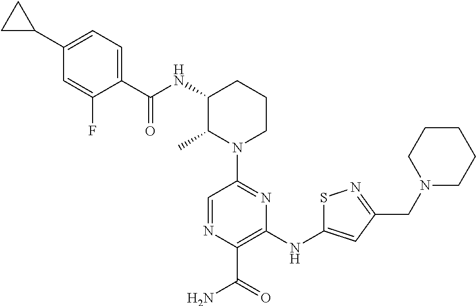 [SMILES: C[C@@H]1[C@H](NC(=O)c2ccc(C3CC3)cc2F)CCCN1c1cnc(C(N)=O)c(Nc2cc(CN3CCCCC3)ns2)n1] | 5-[(2R,3R)-3-(4-cyclopropyl-2- fluorobenzamido)-2- methylpiperidin-1-yl]-3-{[3- (piperidin-1-ylmethyl)- 1,2-thiazol- 5-yl]amino}pyrazine-2- carboxamide |
| A-137 | 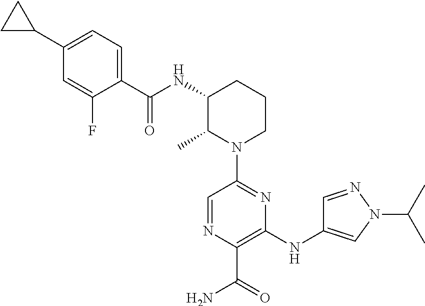 [SMILES: CC(C)n1cc(Nc2nc(N3CCC[C@@H](NC(=O)c4ccc(C5CC5)cc4F)[C@H]3C)cnc2C(N)=O)cn1] | 5-[(2R,3R)-3-(4-cyclopropyl-2- fluorobenzamido)-2- methylpiperidin-1-yl]-3-{[1- (propan-2-yl)-1H-pyrazol-4- yl]amino}pyrazine-2- carboxamide |
| A-138 | 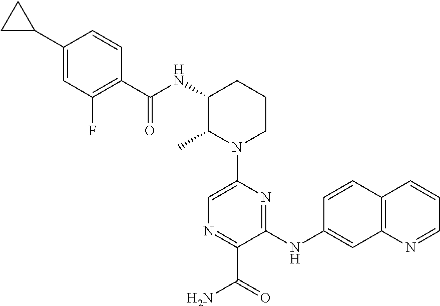 [SMILES: C[C@@H]1[C@H](NC(=O)c2ccc(C3CC3)cc2F)CCCN1c1cnc(C(N)=O)c(Nc2ccc3cccnc3c2)n1] | 5-[(2R,3R)-3-(4-cyclopropyl-2- fluorobenzamido)-2- methylpiperidin-1-yl]- 3-[(quinolin- 7-yl)amino]pyrazine-2- carboxamide |
| A-139 | 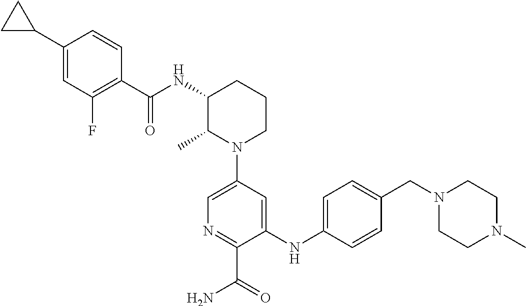 [SMILES: C[C@@H]1[C@H](NC(=O)c2ccc(C3CC3)cc2F)CCCN1c1cnc(C(N)=O)c(Nc2ccc(CN3CCN(C)CC3)cc2)c1] | 5-[(2R,3R)-3-(4-cyclopropyl-2- fluorobenzamido)-2- methylpiperidin-1-yl]-3-({4-[(4- methylpiperazin-1- yl)methyl]phenyl}amino)pyri- dine-2-carboxamide |
| A-140 | 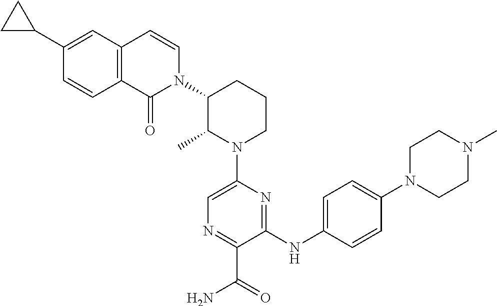 [SMILES: C[C@@H]1[C@H](n2ccc3cc(C4CC4)ccc3c2=O)CCCN1c1cnc(C(N)=O)c(Nc2ccc(N3CCN(C)CC3)cc2)n1] | 5-[(2R,3R)-3-(6-cyclopropyl-1- oxo-1,2-dihydro- isoquinolin-2-yl)- 2-methylpiperidin-1-yl]-3-{[4- (4-methylpiperazin-1- yl)phenyl]amino}pyrazine-2- carboxamide |
| A-141 | 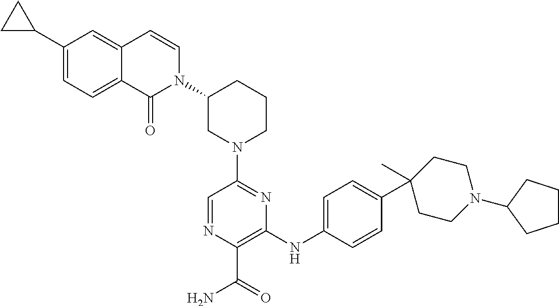 [SMILES: CC1(c2ccc(Nc3nc(N4CCC[C@@H](n5ccc6cc(C7CC7)ccc6c5=O)C4)cnc3C(N)=O)cc2)CCN(C2CCCC2)CC1] | 3-{[4-(1-cyclopentyl-4- methylpiperidin-4- yl)phenyl]amino}-5-[(3R)-3-(6- cyclopropyl-1-oxo-1,2- dihydroisoquinolin- 2-yl)piperidin- 1-yl]pyrazine-2-carboxamide |
| A-142 | 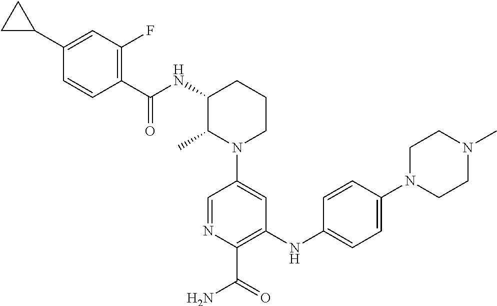 [SMILES: C[C@@H]1[C@H](NC(=O)c2ccc(C3CC3)cc2F)CCCN1c1cnc(C(N)=O)c(Nc2ccc(N3CCN(C)CC3)cc2)c1] | 5-[(2R,3R)-3-(4-cyclopropyl-2- fluorobenzamido)-2- methylpiperidin-1-yl]-3-{[4-(4- methylpiperazin-1- yl)phenyl]amino}pyridine-2- carboxamide |
| A-143 | 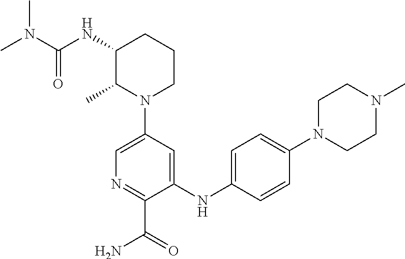 | 5-[(2R,3R)-3- [(dimethylcarbamoyl)amino]-2- methylpiperidin-1-yl]-3-{[4-(4- methylpiperazin-1- yl)phenyl]amino}pyridine-2- carboxamide |
| A-144 | 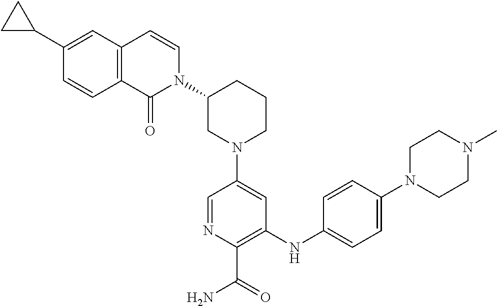 [SMILES: CN1CCN(c2ccc(Nc3cc(N4CCC[C@@H](n5ccc6cc(C7CC7)ccc6c5=O)C4)cnc3C(N)=O)cc2)CC1] | (R)-5-(3-(6-cyclopropyl-1- oxoiso- quinolin-2(1H)-yl)piperidin- 1-yl)-3-(4-(4-methylpiperazin-1- yl)phenylamino)picolinamide |
| A-145 | 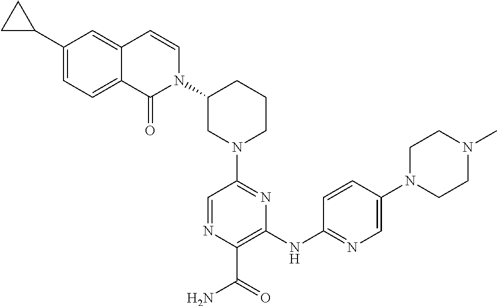 [SMILES: CN1CCN(c2ccc(Nc3nc(N4CCC[C@@H](n5ccc6cc(C7CC7)ccc6c5=O)C4)cnc3C(N)=O)nc2)CC1] | 5-[(3R)-3-(6-cyclopropyl-1-oxo- 1,2-dihydroisoquinolin-2- yl)piperidin-1-yl]-3-{[5-(4- methylpiperazin-1-yl)pyridin-2- yl]amino}pyrazine-2- carboxamide |
| A-146 | 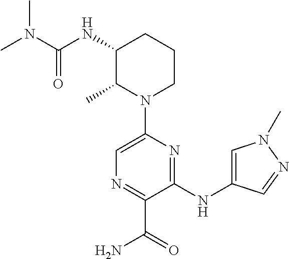 [SMILES: C[C@@H]1[C@H](NC(=O)N(C)C)CCCN1c1cnc(C(N)=O)c(Nc2cnn(C)c2)n1] | 5-[(2R,3R)-3- [(dimethylcarbamoyl)amino]-2- methylpiperidin-1-yl]-3-[(1- methyl-1H-pyrazol-4- yl)amino]pyrazine-2- carboxamide |
| A-147 | 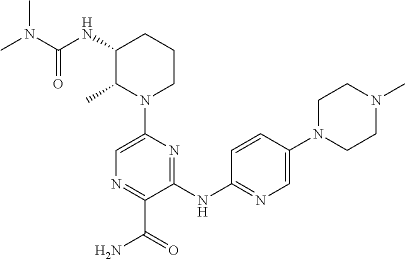 | 5-[(2R,3R)-3- [(dimethylcarbamoyl)amino]-2- methylpiperidin-1-yl]-3-{[5-(4- methylpiperazin-1-yl)pyridin-2- yl]amino}pyrazine-2- carboxamide |
| A-148 | 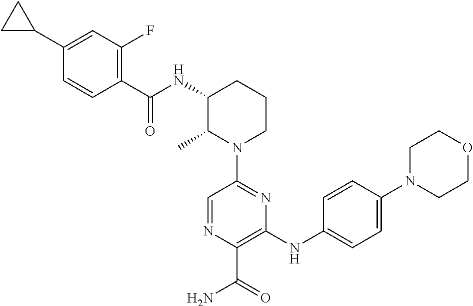 [SMILES: C[C@@H]1[C@H](NC(=O)c2ccc(C3CC3)cc2F)CCCN1c1cnc(C(N)=O)c(Nc2ccc(N3CCOCC3)cc2)n1] | 5-[(2R,3R)-3-(4-cyclopropyl-2- fluorobenzamido)-2- methylpiperidin-1-yl]-3-{[4- (morpholin-4- yl)phenyl]amino}pyrazine-2- carboxamide |
| A-149 | 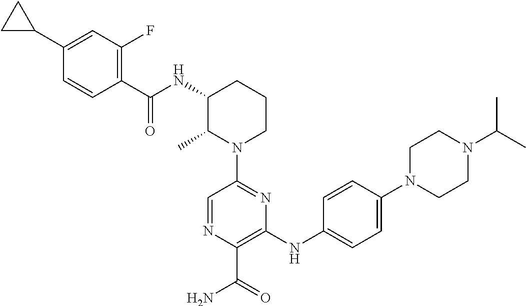 [SMILES: CC(C)N1CCN(c2ccc(Nc3nc(N4CCC[C@@H](NC(=O)c5ccc(C6CC6)cc5F)[C@H]4C)cnc3C(N)=O)cc2)CC1] | 5-[(2R,3R)-3-(4-cyclopropyl-2- fluorobenzamido)-2- methylpiperidin-1-yl]-3-({4-[4- (propan-2-yl)piperazin-1- yl]phenyl}amino)pyrazine-2- carboxamide |
| A-150 | 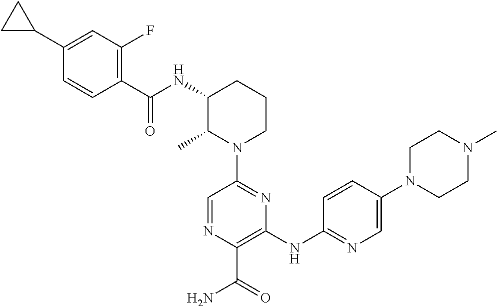 [SMILES: C[C@@H]1[C@H](NC(=O)c2ccc(C3CC3)cc2F)CCCN1c1cnc(C(N)=O)c(Nc2ccc(N3CCN(C)CC3)cn2)n1] | 5-[(2R,3R)-3-(4-cyclopropyl-2- fluorobenzamido)-2- methylpiperidin-1-yl]-3-{[5-(4- methylpiperazin-1-yl)pyridin-2- yl]amino}pyrazine-2- carboxamide |
| A-151 | 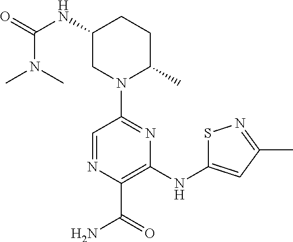 [SMILES: Cc1cc(Nc2nc(N3C[C@H](NC(=O)N(C)C)CC[C@@H]3C)cnc2C(N)=O)sn1] | 5-[(2S,5R)-5- [(dimethylcarbamoyl)amino]-2- methylpiperidin-1-yl]-3-[(3- methyl-1,2-thiazol-5- yl)amino]pyrazine-2- carboxamide |
| A-152 | 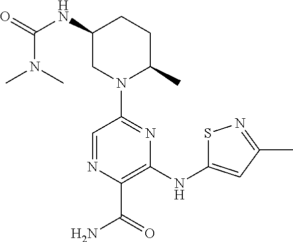 [SMILES: Cc1cc(Nc2nc(N3C[C@@H](NC(=O)N(C)C)CC[C@H]3C)cnc2C(N)=O)sn1] | 5-[(2R,5S)-5- [(dimethylcarbamoyl)amino]-2- methylpiperidin-1-yl]-3-[(3- methyl-1,2-thiazol-5- yl)amino]pyrazine-2- carboxamide |
| A-153 | 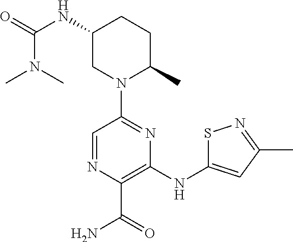 [SMILES: Cc1cc(Nc2nc(N3C[C@H](NC(=O)N(C)C)CC[C@H]3C)cnc2C(N)=O)sn1] | 5-[(2R,5R)-5- [(dimethylcarbamoyl)amino]-2- methylpiperidin-1-yl]-3-[(3- methyl-1,2-thiazol-5- yl)amino]pyrazine-2- carboxamide |
| A-154 | 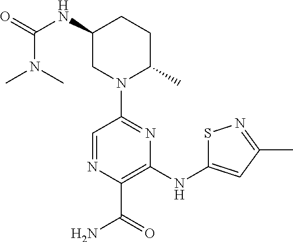 [SMILES: Cc1cc(Nc2nc(N3C[C@@H](NC(=O)N(C)C)CC[C@@H]3C)cnc2C(N)=O)sn1] | 5-[(2S,5S)-5- [(dimethylcarbamoyl)amino]-2- methylpiperidin-1-yl]-3-[(3- methyl-1,2-thiazol-5- yl)amino]pyrazine-2- carboxamide |
| A-155 | 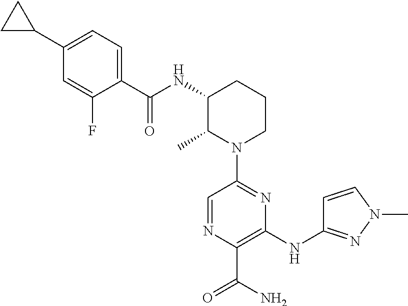 [SMILES: C[C@@H]1[C@H](NC(=O)c2ccc(C3CC3)cc2F)CCCN1c1cnc(C(N)=O)c(Nc2ccn(C)n2)n1] | 5-[(2R,3R)-3-(4-cyclopropyl-2- fluorobenzamido)-2- methylpiperidin-1-yl]-3-[(1- methyl-1H-pyrazol-3- yl)amino]pyrazine-2- carboxamide |
| A-156 | 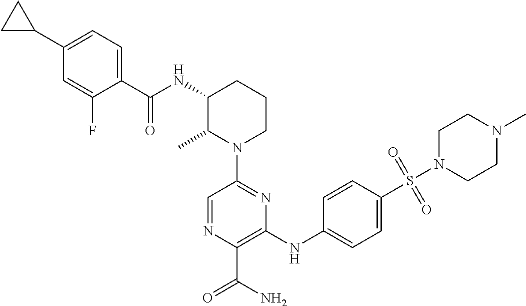 [SMILES: C[C@@H]1[C@H](NC(=O)c2ccc(C3CC3)cc2F)CCCN1c1cnc(C(N)=O)c(Nc2ccc(S(=O)(=O)N3CCN(C)CC3)cc2)n1] | 5-[(2R,3R)-3-(4-cyclopropyl-2- fluorobenzamido)-2- methylpiperidin-1-yl]-3-({4-[(4- methylpiperazin-1- yl)sulfonyl]phenyl}amino)pyr- azine-2-carboxamide |
| A-157 | 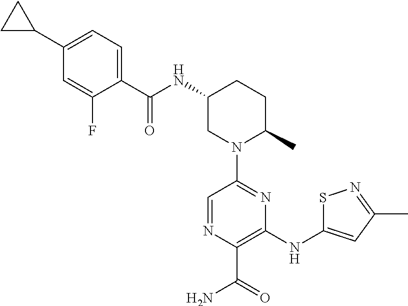 [SMILES: Cc1cc(Nc2nc(N3C[C@H](NC(=O)c4ccc(C5CC5)cc4F)CC[C@H]3C)cnc2C(N)=O)sn1] | 5-[(2R,5R)-5-(4-cyclopropyl-2- fluorobenzamido)-2- methylpiperidin-1-yl]-3-[(3- methyl-1,2-thiazol-5- yl)amino]pyrazine-2- carboxamide |
| A-158 | 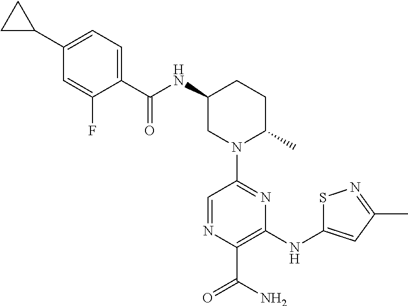 [SMILES: Cc1cc(Nc2nc(N3C[C@@H](NC(=O)c4ccc(C5CC5)cc4F)CC[C@@H]3C)cnc2C(N)=O)sn1] | 5-[(2S,5S)-5-(4-cyclopropyl-2- fluorobenzamido)-2- methylpiperidin-1-yl]-3-[(3- methyl-1,2-thiazol-5- yl)amino]pyrazine-2- carboxamide |
| A-159 | 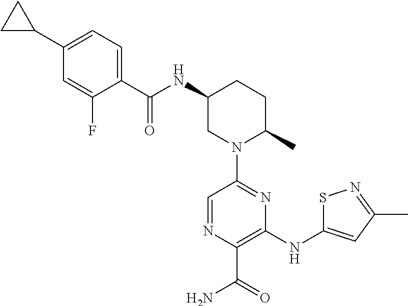 [SMILES: Cc1cc(Nc2nc(N3C[C@@H](NC(=O)c4ccc(C5CC5)cc4F)CC[C@H]3C)cnc2C(N)=O)sn1] | 5-[(2R,5S)-5-(4-cyclopropyl-2- fluorobenzamido)-2- methylpiperidin-1-yl]-3-[(3- methyl-1,2-thiazol-5- yl)amino]pyrazine-2- carboxamide |
| A-160 | 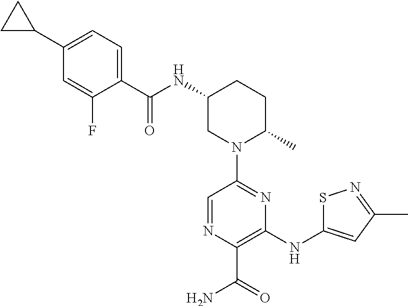 [SMILES: Cc1cc(Nc2nc(N3C[C@H](NC(=O)c4ccc(C5CC5)cc4F)CC[C@@H]3C)cnc2C(N)=O)sn1] | 5-[(2S,5R)-5-(4-cyclopropyl-2- fluorobenzamido)-2- methylpiperidin-1-yl]-3-[(3- methyl-1,2-thiazol-5- yl)amino]pyrazine-2- carboxamide |
| A-161 | 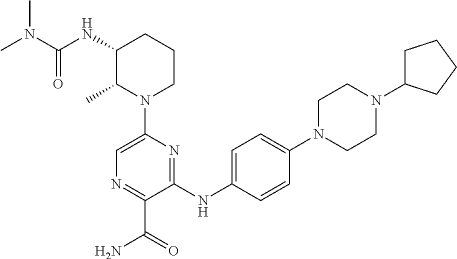 [SMILES: C[C@@H]1[C@H](NC(=O)N(C)C)CCCN1c1cnc(C(N)=O)c(Nc2ccc(N3CCN(C4CCCC4)CC3)cc2)n1] | 3-{[4-(4-cyclopentyl- piperazin-1- yl)phenyl]amino}- 5-[(2R,3R)-3-[(dimethyl- carbamoyl)amino]-2- methylpiperidin-1-yl]pyrazine- 2-carboxamide |
| A-162 | 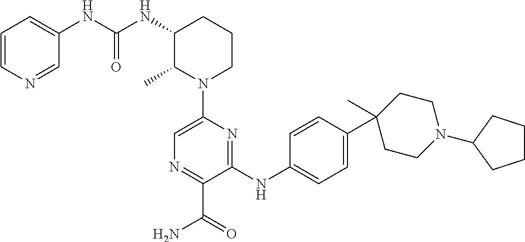 [SMILES: C[C@@H]1[C@H](NC(=O)Nc2cccnc2)CCCN1c1cnc(C(N)=O)c(Nc2ccc(C3(C)CCN(C4CCCC4)CC3)cc2)n1] | 3-{[4-(1-cyclopentyl-4- methylpiperidin-4- yl)phenyl]amino}-5-[(2R,3R)- 2-methyl-3-{[(pyridin-3- yl)carbamoyl]amino}piperidin- 1-yl]pyrazine-2-carboxamide |
| A-163 | 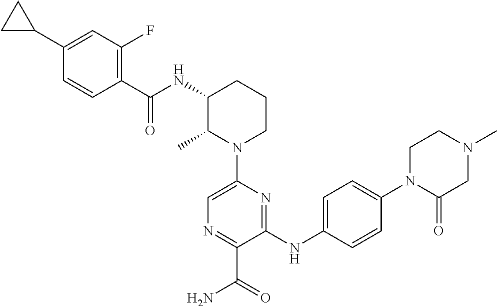 [SMILES: C[C@@H]1[C@H](NC(=O)c2ccc(C3CC3)cc2F)CCCN1c1cnc(C(N)=O)c(Nc2ccc(N3CCN(C)CC3=O)cc2)n1] | 5-[(2R,3R)-3-(4-cyclopropyl-2- fluorobenzamido)-2- methylpiperidin-1-yl]-3-{[4-(4- methyl-2-oxopiperazin-1- yl)phenyl]amino}pyrazine-2- carboxamide |
| A-164 | 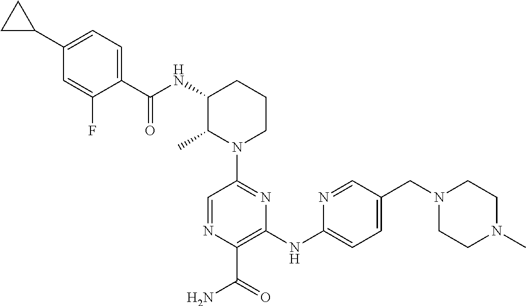 [SMILES: C[C@@H]1[C@H](NC(=O)c2ccc(C3CC3)cc2F)CCCN1c1cnc(C(N)=O)c(Nc2ccc(CN3CCN(C)CC3)cn2)n1] | 5-[(2R,3R)-3-(4-cyclopropyl-2- fluorobenzamido)-2- methylpiperidin-1-yl]-3-({5-[(4- methylpiperazin-1- yl)methyl]pyridin-2- yl}amino)pyrazine-2- carboxamide |
| A-165 | 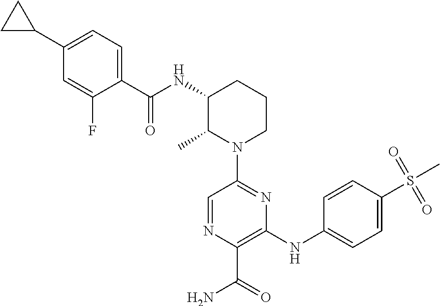 [SMILES: C[C@@H]1[C@H](NC(=O)c2ccc(C3CC3)cc2F)CCCN1c1cnc(C(N)=O)c(Nc2ccc(S(C)(=O)=O)cc2)n1] | 5-[(2R,3R)-3-(4-cyclopropyl-2- fluorobenzamido)-2- methylpiperidin-1-yl]-3-[(4- methanesulfonyl- phenyl)amino]pyr- azine-2-carboxamide |
| A-166 | 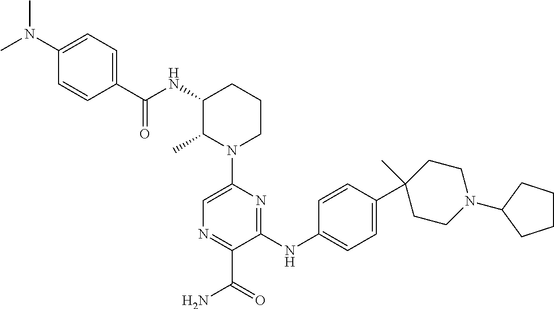 [SMILES: C[C@@H]1[C@H](NC(=O)c2ccc(N(C)C)cc2)CCCN1c1cnc(C(N)=O)c(Nc2ccc(C3(C)CCN(C4CCCC4)CC3)cc2)n1] | 3-{[4-(1-cyclopentyl-4- methylpiperidin-4- yl)phenyl]amino}- 5-[(2R,3R)-3-[4- (dimethylamino)benzamido]-2- methylpiperidin-1-yl]pyrazine- 2-carboxamide |
| A-167 | 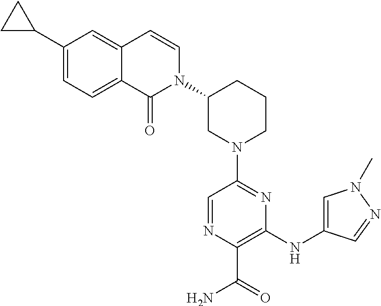 [SMILES: Cn1cc(Nc2nc(N3CCC[C@@H](n4ccc5cc(C6CC6)ccc5c4=O)C3)cnc2C(N)=O)cn1] | 5-[(3R)-3-(6-cyclopropyl-1-oxo- 1,2-dihydroisoquinolin-2- yl)piperidin-1-yl]-3- [(1-methyl-1H- pyrazol-4-yl)amino]pyrazine-2- carboxamide |
| A-168 | 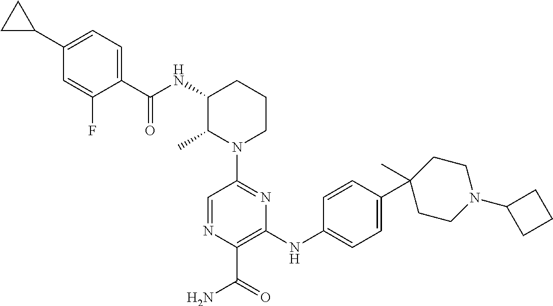 [SMILES: C[C@@H]1[C@H](NC(=O)c2ccc(C3CC3)cc2F)CCCN1c1cnc(C(N)=O)c(Nc2ccc(C3(C)CCN(C4CCC4)CC3)cc2)n1] | 3-{[4-(1-cyclobutyl-4- methylpiperidin-4- yl)phenyl]amino}- 5-[(2R,3R)-3-(4- cyclopropyl-2-fluoro- benzamido)-2- methylpiperidin-1-yl]pyrazine- 2-carboxamide |
| A-169 | 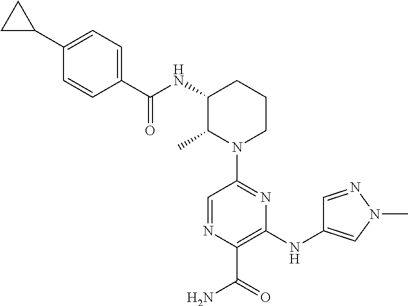 [SMILES: C[C@@H]1[C@H](NC(=O)c2ccc(C3CC3)cc2)CCCN1c1cnc(C(N)=O)c(Nc2cnn(C)c2)n1] | 5-[(2R,3R)-3-(4- cyclopropylbenzamido)-2- methylpiperidin-1-yl]-3-[(1- methyl-1H-pyrazol-4- yl)amino]pyrazine-2- carboxamide |
| A-170 | 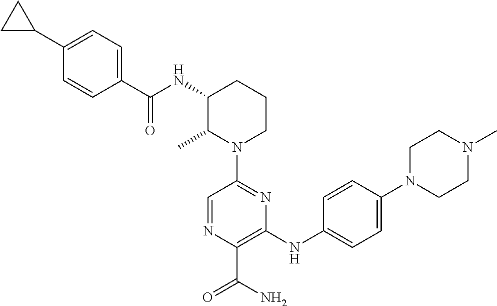 [SMILES: C[C@@H]1[C@H](NC(=O)c2ccc(C3CC3)cc2)CCCN1c1cnc(C(N)=O)c(Nc2ccc(N3CCN(C)CC3)cc2)n1] | 5-[(2R,3R)-3-(4- cyclopropylbenzamido)-2- methylpiperidin-1-yl]-3-{[4-(4- methylpiperazin-1- yl)phenyl]amino}pyrazine-2- carboxamide |
| A-171 | 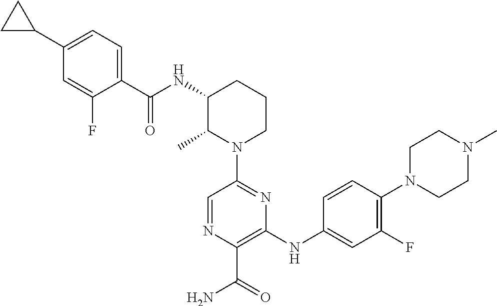 [SMILES: C[C@@H]1[C@H](NC(=O)c2ccc(C3CC3)cc2F)CCCN1c1cnc(C(N)=O)c(Nc2ccc(N3CCN(C)CC3)c(F)c2)n1] | 5-[(2R,3R)-3-(4-cyclopropyl-2- fluorobenzamido)-2- methylpiperidin-1- yl]-3-{[3-fluoro- 4-(4-methylpiperazin-1- yl)phenyl]amino}pyrazine-2- carboxamide |
| A-172 | 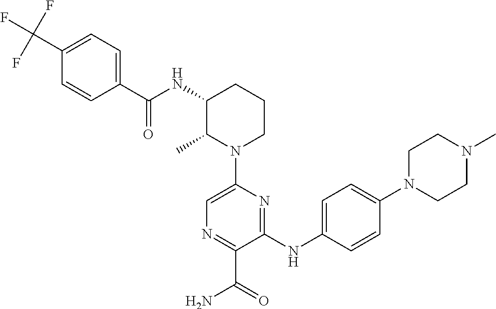 [SMILES: C[C@@H]1[C@H](NC(=O)c2ccc(C(F)(F)F)cc2)CCCN1c1cnc(C(N)=O)c(Nc2ccc(N3CCN(C)CC3)cc2)n1] | 5-[(2R,3R)-2-methyl-3-[4- (trifluoromethyl)benz- amido]piperidin- 1-yl]-3-{4-(4-methylpiperazin- 1-yl)phenyl]amino}pyrazine-2- carboxamide |
| A-173 | 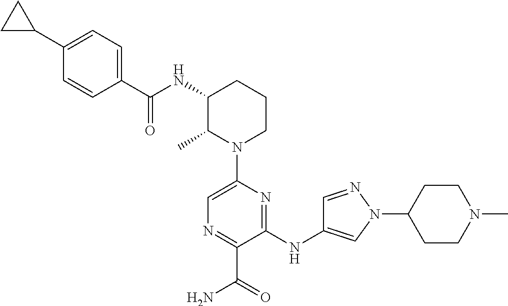 [SMILES: C[C@@H]1[C@H](NC(=O)c2ccc(C3CC3)cc2)CCCN1c1cnc(C(N)=O)c(Nc2cnn(C3CCN(C)CC3)c2)n1] | 5-[(2R,3R)-3-(4- cyclopropylbenzamido)-2- methylpiperidin-1-yl]-3-{1-(1- methylpiperidin- 4-yl)-1H-pyrazol- 4-yl]amino}pyrazine-2- carboxamide |
| A-174 | 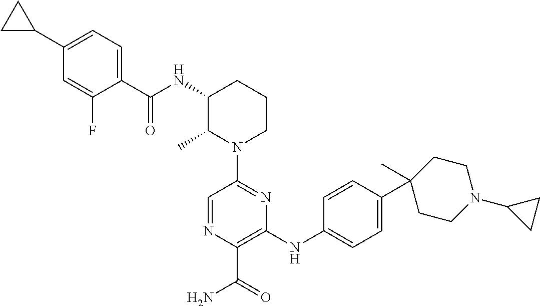 [SMILES: C[C@@H]1[C@H](NC(=O)c2ccc(C3CC3)cc2F)CCCN1c1cnc(C(N)=O)c(Nc2ccc(C3(C)CCN(C4CC4)CC3)cc2)n1] | 5-[(2R,3R)-3-(4-cyclopropyl-2- fluorobenzamido)-2- methylpiperidin-1-yl]-3-{[4-(1- cyclopropyl-4-methylpiperidin- 4-yl)phenyl]amino}pyrazine-2- carboxamide |
| A-175 | 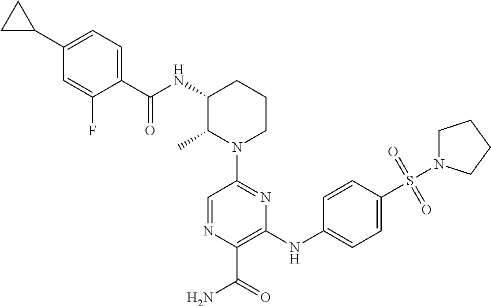 [SMILES: C[C@@H]1[C@H](NC(=O)c2ccc(C3CC3)cc2F)CCCN1c1cnc(C(N)=O)c(Nc2ccc(S(=O)(=O)N3CCCC3)cc2)n1] | 5-[(2R,3R)-3-(4-cyclopropyl- 2-fluorobenzamido)-2- methylpiperidin-1-yl]-3-{[4- (pyrrolidine-1- sulfonyl)phenyl]amino}pyr- azine-2-carboxamide |
| A-176 | 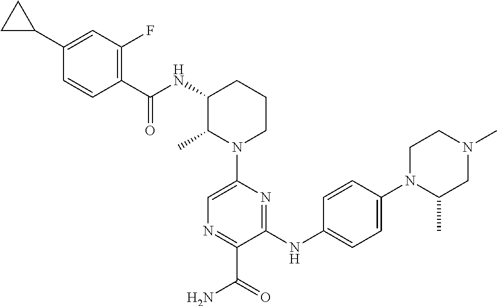 [SMILES: C[C@@H]1[C@H](NC(=O)c2ccc(C3CC3)cc2F)CCCN1c1cnc(C(N)=O)c(Nc2ccc(N3CCN(C)C[C@@H]3C)cc2)n1] | 5-[(2R,3R)-3-(4-cyclopropyl-2- fluorobenzamido)-2- methylpiperidin-1-yl]-3-({4- [(2S)-2,4-dimethylpiperazin-1- yl]phenyl}amino)pyrazine-2- carboxamide |
| A-177 | 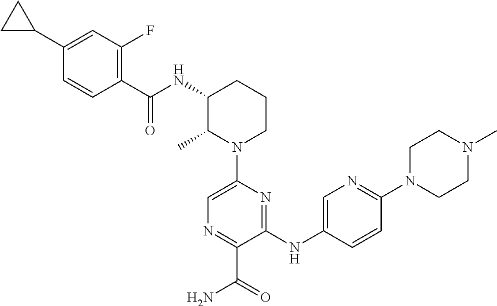 [SMILES: C[C@@H]1[C@H](NC(=O)c2ccc(C3CC3)cc2F)CCCN1c1cnc(C(N)=O)c(Nc2ccc(N3CCN(C)CC3)nc2)n1] | 5-[(2R,3R)-3-(4-cyclopropyl-2- fluorobenzamido)-2- methylpiperidin-1-yl]-3-{[6-(4- methylpiperazin-1-yl)pyridin-3- yl]amino}pyrazine-2- caoxamide |
| A-178 | 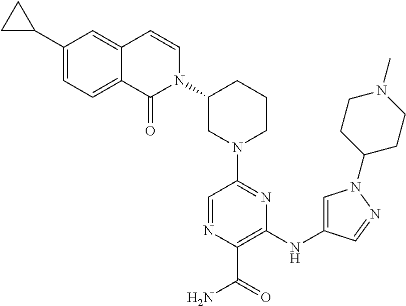 [SMILES: CN1CCC(n2cc(Nc3nc(N4CCC[C@@H](n5ccc6cc(C7CC7)ccc6c5=O)C4)cnc3C(N)=O)cn2)CC1] | 5-[(3R)-3-(6-cyclopropyl- 1-oxo- 1,2-dihydroisoquinolin-2- yl)piperidin-1-yl]-3-{[1-(1- methylpiperidin-4-yl)- 1H-pyrazol- 4-yl]amino}pyrazine-2- carboxamide |
| A-179 | 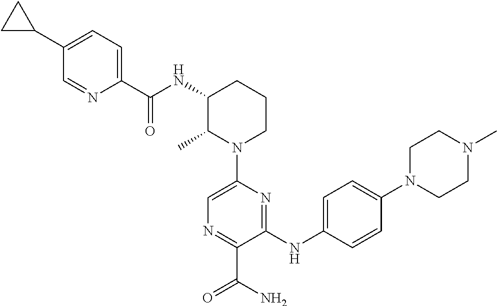 [SMILES: C[C@@H]1[C@H](NC(=O)c2ccc(C3CC3)cn2)CCCN1c1cnc(C(N)=O)c(Nc2ccc(N3CCN(C)CC3)cc2)n1] | 5-[(2R,3R)-3-(5- cyclopropyl- pyridine-2-amido)-2- methylpiperidin-1-yl]-3-{[4-(4- methylpiperazin-1- yl)phenyl]amino}pyrazine-2- carboxamide |
| A-180 | 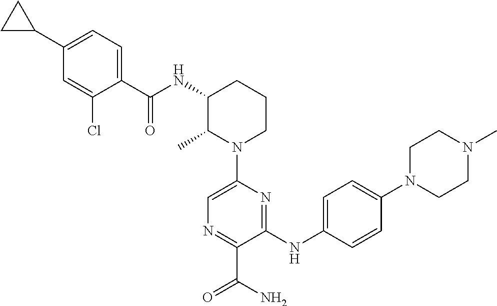 [SMILES: C[C@@H]1[C@H](NC(=O)c2ccc(C3CC3)cc2Cl)CCCN1c1cnc(C(N)=O)c(Nc2ccc(N3CCN(C)CC3)cc2)n1] | 5-[(2R,3R)-3-(2-chloro-4- cyclopropylbenzamido)-2- methylpiperidin-1-yl]-3-{[4-(4- methylpiperazin-1- yl)phenyl]amino}pyrazine-2- carboxamide |
| A-181 | 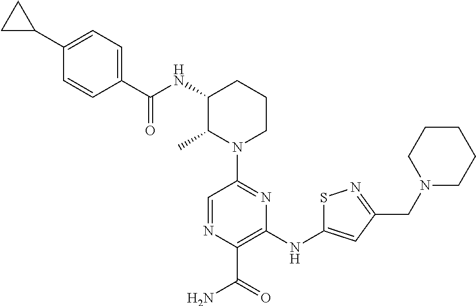 [SMILES: C[C@@H]1[C@H](NC(=O)c2ccc(C3CC3)cc2)CCCN1c1cnc(C(N)=O)c(Nc2cc(CN3CCCCC3)ns2)n1] | 5-[(2R,3R)-3-(4- cyclopropylbenzamido)-2- methylpiperidin-1-yl]-3-{[3- (piperidin-1-ylmethyl)- 1,2-thiazol- 5-yl]amino}pyrazine-2- carboxamide |
| A-182 | 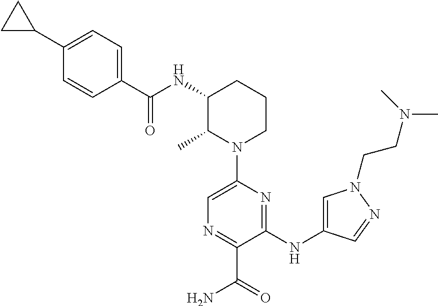 [SMILES: C[C@@H]1[C@H](NC(=O)c2ccc(C3CC3)cc2)CCCN1c1cnc(C(N)=O)c(Nc2cnn(CCN(C)C)c2)n1] | 5-[(2R,3R)-3-(4- cyclopropylbenzamido)-2- methylpiperidin-1-yl]-3-({1-[2- (dimethylamino)ethyl]- 1H-pyrazol- 4-yl}amino)pyrazine-2- carboxamide |
| A-183 | 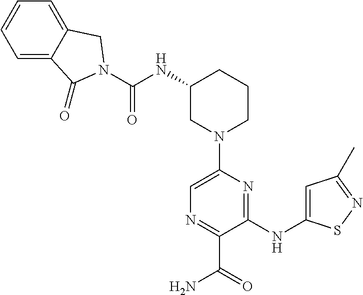 [SMILES: Cc1cc(Nc2nc(N3CCC[C@@H](NC(=O)N4Cc5ccccc5C4=O)C3)cnc2C(N)=O)sn1] | N-[(3R)-1-{5-carbamoyl-6-[(3- methyl-1,2-thiazol-5- yl)amino]pyrazin- 2-yl}piperidin-3- yl]-1-oxo-2,3-dihydro-1H- isoindole-2-carboxamide |
| A-184 | 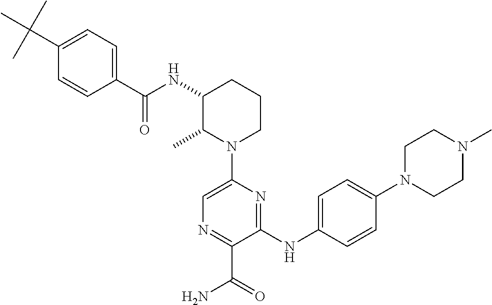 | 5-[(2R,3R)-3-(4-tert- butylbenzamido)-2- methylpiperidin-1-yl]-3-{[4-(4- methylpiperazin-1- yl)phenyl]amino}pyrazine-2- carboxamide |
| A-185 | 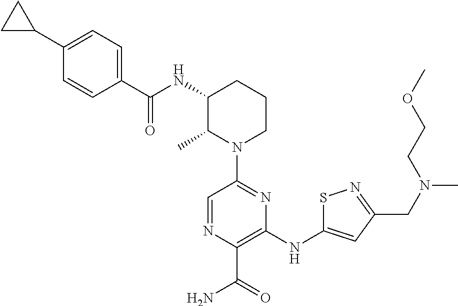 [SMILES: COCCN(C)Cc1cc(Nc2nc(N3CCC[C@@H](NC(=O)c4ccc(C5CC5)cc4)[C@H]3C)cnc2C(N)=O)sn1] | 5-((2R,3R)-3-(4- cyclopropylbenzamido)-2- methylpiperidin-1-yl)-3-(3- (((2-methoxyethyl)(meth- yl)amino)meth- yl)isothiazol-5-ylamino)pyr- azine-2-carboxamide |
| A-186 | 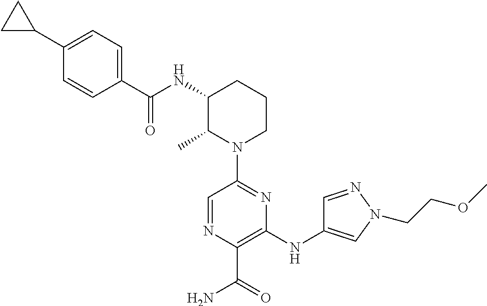 [SMILES: COCCn1cc(Nc2nc(N3CCC[C@@H](NC(=O)c4ccc(C5CC5)cc4)[C@H]3C)cnc2C(N)=O)cn1] | 5-((2R,3R)-3-(4- cyclopropylbenzamido)-2- methylpiperidin-1-yl)-3-(1-(2- methoxylethyl)-1H-pyrazol-4- ylamino)pyrazine-2- carboxamide |
| A-187 | 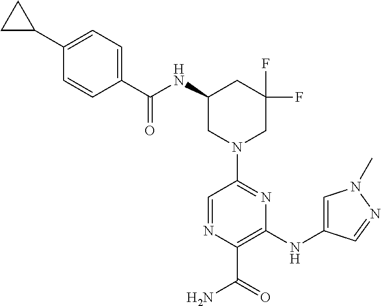 [SMILES: Cn1cc(Nc2nc(N3C[C@@H](NC(=O)c4ccc(C5CC5)cc4)CC(F)(F)C3)cnc2C(N)=O)cn1] | (S)-5-(5-(4- cyclopropylbenzamido)-3,3- difluoropiperidin-1-yl)-3-(1- methyl-1H-pyrazol-4- ylamino)pyrazine-2- carboxamide |
| A-188 | 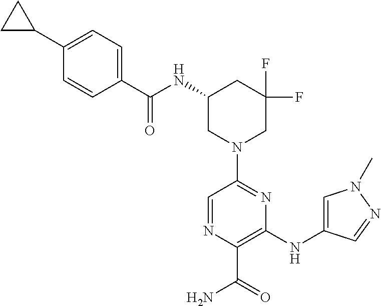 [SMILES: Cn1cc(Nc2nc(N3C[C@H](NC(=O)c4ccc(C5CC5)cc4)CC(F)(F)C3)cnc2C(N)=O)cn1] | (R)-5-(5-(4- cyclopropylbenzamido)-3,3- difluoropiperidin-1-yl)-3-(1- methyl-1H-pyrazol-4- ylamino)pyrazine-2- carboxamide |
| A-189 | 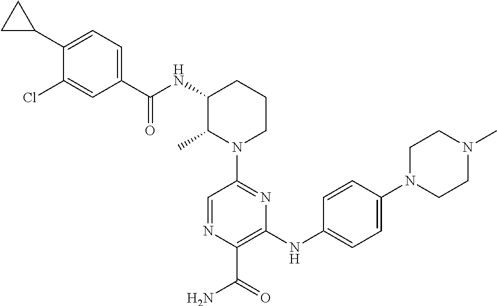 [SMILES: C[C@@H]1[C@H](NC(=O)c2ccc(C3CC3)c(Cl)c2)CCCN1c1cnc(C(N)=O)c(Nc2ccc(N3CCN(C)CC3)cc2)n1] | 5-((2R,3R)-3-(3-chloro-4- cyclopropylbenzamido)-2- methylpiperidin-1-yl)-3-(4-(4- methylpiperazin-1- yl)phenylamino)pyrazine-2- carboxamide |
| A-190 | 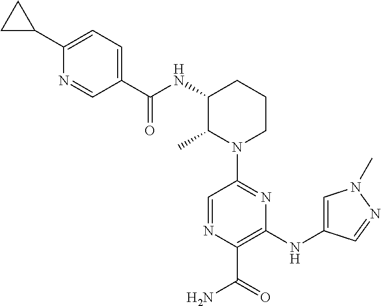 [SMILES: C[C@@H]1[C@H](NC(=O)c2ccc(C3CC3)nc2)CCCN1c1cnc(C(N)=O)c(Nc2cnn(C)c2)n1] | 5-((2R,3R)-3-(6- cyclopropylnicotinamido)-2- methylpiperidin- 1-yl)-3-(1-methyl- 1H-pyrazol-4-ylamino)pyr- azine-2-carboxamide |
| A-191 | 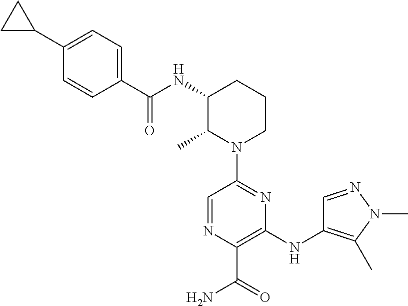 [SMILES: Cc1c(Nc2nc(N3CCC[C@@H](NC(=O)c4ccc(C5CC5)cc4)[C@H]3C)cnc2C(N)=O)cnn1C] | 5-((2R,3)R-3-(4- cyclopropylbenzamido)-2- methylpiperidin-1-yl)-3-(1,5- dimethyl-1H-pyrazol-4- ylamino)pyrazine-2- carboxamide |
| A-192 | 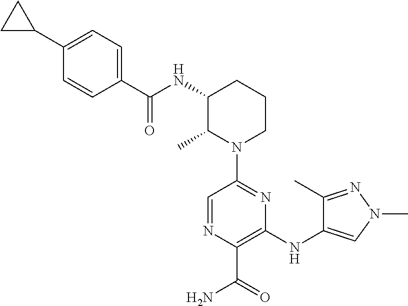 [SMILES: Cc1nn(C)cc1Nc1nc(N2CCC[C@@H](NC(=O)c3ccc(C4CC4)cc3)[C@H]2C)cnc1C(N)=O] | 5-((2R,3R)-3-(4- cyclopropylbenzamido)-2- methylpiperidin-1-yl)-3-(1,3- dimethyl-1H-pyrazol-4- ylamino)pyrazine-2- carboxamide |
| A-193 | 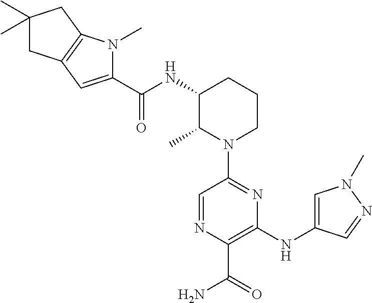 [SMILES: C[C@@H]1[C@H](NC(=O)c2cc3c(n2C)CC(C)(C)C3)CCCN1c1cnc(C(N)=O)c(Nc2cnn(C)c2)n1] | N-((2R,3R)-1-(5- carbamoyl-6-(1- methyl-1H-pyrazol-4- ylamino)pyrazin-2-yl)-2- methylpiperidin-3-yl)-1,5,5- trimethyl-1,4,5,6- tetrahydrocyclo- penta[b]pyrrole- 2-carboxamide |
| A-194 | 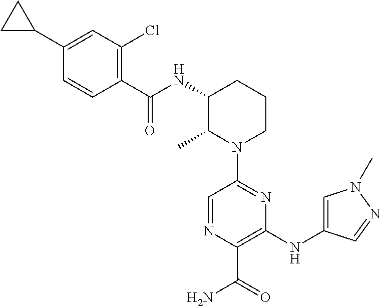 [SMILES: C[C@@H]1[C@H](NC(=O)c2ccc(C3CC3)cc2Cl)CCCN1c1cnc(C(N)=O)c(Nc2cnn(C)c2)n1] | 5-((2R,3R)-3-(2-chloro-4- cyclopropylbenzamido)-2- methylpiperidin- 1-yl)-3-(1-methyl- 1H-pyrazol-4- ylamino)pyrazine-2- carboxamide |
| A-195 | 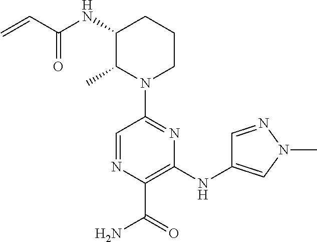 [SMILES: C=CC(=O)N[C@@H]1CCCN(c2cnc(C(N)=O)c(Nc3cnn(C)c3)n2)[C@@H]1C] | 5-((2R,3R)-3-acrylamido-2- methylpiperidin-1-yl)- 3-(1-methyl-1H- pyrazol-4-ylamino)pyrazine-2- carboxamide |
| A-196 | 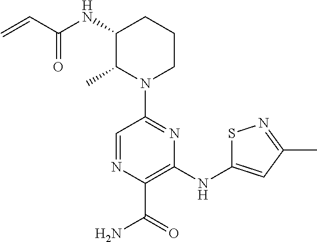 [SMILES: C=CC(=O)N[C@@H]1CCCN(c2cnc(C(N)=O)c(Nc3cc(C)ns3)n2)[C@@H]1C] | 5-((2R,3R)-3-acrylamido-2- methylpiperidin-1-yl)-3-(3- methylisothiazol-5- ylamino)pyrazine-2- carboxamide |
| A-197 | 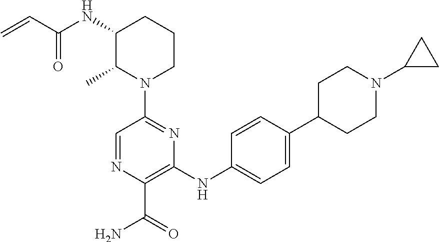 [SMILES: C=CC(=O)N[C@@H]1CCCN(c2cnc(C(N)=O)c(Nc3ccc(C4CCN(C5CC5)CC4)cc3)n2)[C@@H]1C] | 5-((2R,3R)-3-acrylamido-2- methylpiperidin-1-yl)-3-(4-(1- cyclopropylpiperidin-4- yl)phenylamino)pyrazine-2- carboxamide |
| A-198 | 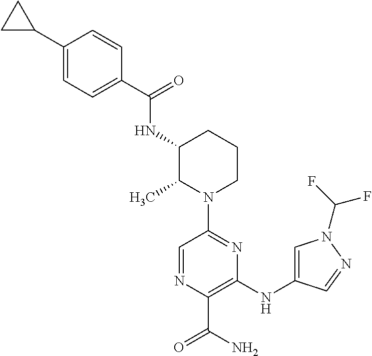 [SMILES: C[C@@H]1[C@H](NC(=O)c2ccc(C3CC3)cc2)CCCN1c1cnc(C(N)=O)c(Nc2cnn(C(F)F)c2)n1] | 5-[(2R,3R)-3-(4- cyclopropylbenzamido)-2- methylpiperidin-1-yl]-3-{[1- (difluoromethyl)-1H- pyrazol-4-yl]amino}pyrazine- 2-carboxamide |
| A-199 | 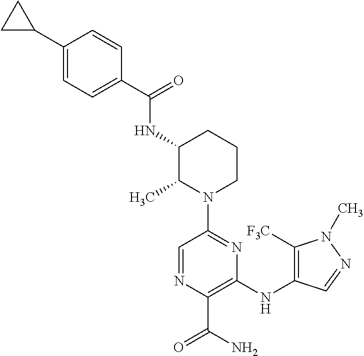 [SMILES: C[C@@H]1[C@H](NC(=O)c2ccc(C3CC3)cc2)CCCN1c1cnc(C(N)=O)c(Nc2cnn(C)c2C(F)(F)F)n1] | 5-[(2R,3R)-3-(4- cyclopropylbenzamido)-2- methylpiperidin-1-yl]-3-{[1- methyl-5- (trifluoromethyl)-1H-pyrazol- 4-yl]amino}pyrazine-2- carboxamide |
| A-200 | 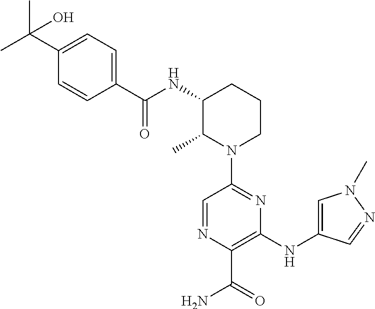 [SMILES: C[C@@H]1[C@H](NC(=O)c2ccc(C(C)(C)O)cc2)CCCN1c1cnc(C(N)=O)c(Nc2cnn(C)c2)n1] | 5-((2R,3R)-3- (4-(2-hydroxypropan- 2-yl)benzamido)-2-methyl- piperidin-1-yl)-3-(1-methyl- 1H-pyrazol-4-yl- amino)pyrazine-2- carboxamide |
| A-201 | 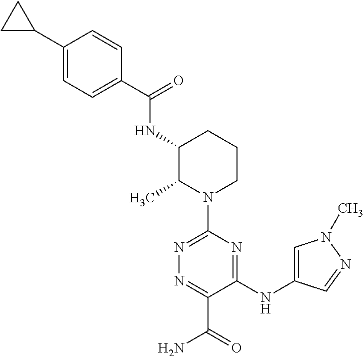 [SMILES: C[C@@H]1[C@H](NC(=O)c2ccc(C3CC3)cc2)CCCN1c1nnc(C(N)=O)c(Nc2cnn(C)c2)n1] | 3-[(2R,3R)-3-(4- cyclopropylbenzamido)-2- methylpiperidin-1-yl]-5-[(1- methyl-1H-pyrazol-4- yl)amino]-1,2,4-triazine-6- carboxamide |
| A-202 | 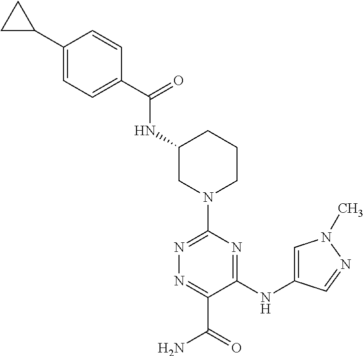 [SMILES: Cn1cc(Nc2nc(N3CCC[C@@H](NC(=O)c4ccc(C5CC5)cc4)C3)nnc2C(N)=O)cn1] | 3-[(3R)-3-(4- cyclopropylbenzamido)piper- idin-1-yl]-5- [(1-methyl-1H-pyrazol-4- yl)amino]-1,2,4-triazine-6- carboxamide |
| A-203 | 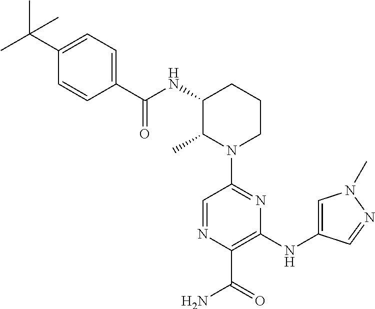 [SMILES: C[C@@H]1[C@H](NC(=O)c2ccc(C(C)(C)C)cc2)CCCN1c1cnc(C(N)=O)c(Nc2cnn(C)c2)n1] | 5-((2R,3R)-3-(4-tert- butylbenzamido)-2- methylpiperidin-1-yl)- 3-(1-methyl-1H-pyrazol- 4-ylamino)pyrazine-2- carboxamide |
| A-204 | 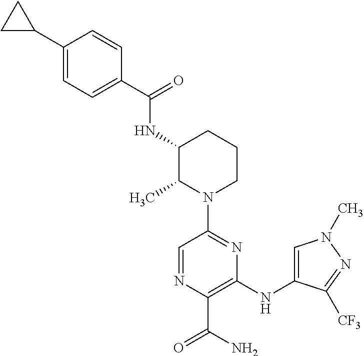 [SMILES: C[C@@H]1[C@H](NC(=O)c2ccc(C3CC3)cc2)CCCN1c1cnc(C(N)=O)c(Nc2cn(C)nc2C(F)(F)F)n1] | 5-[(2R,3R)-3-(4- cyclopropylbenzamido)-2- methylpiperidin-1-yl]-3-{[1- methyl-3-(tri- fluoromethyl)-1H-pyrazol-4- yl]amino}pyrazine-2- carboxamide |
| A-205 | 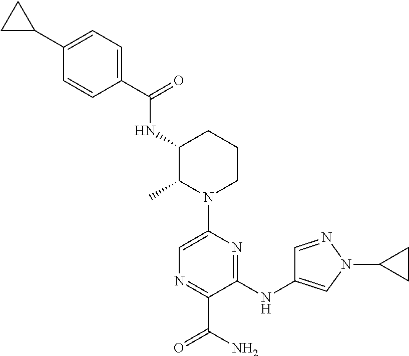 [SMILES: C[C@@H]1[C@H](NC(=O)c2ccc(C3CC3)cc2)CCCN1c1cnc(C(N)=O)c(Nc2cnn(C3CC3)c2)n1] | 3-[(1-cyclopropyl-1H- pyrazol-4-yl)amino]-5- [(2R,3R)-3-(4- cyclopropylbenzamido)-2- methylpiperidin-1-yl]pyrazine- 2-carboxamide |
| A-206 | 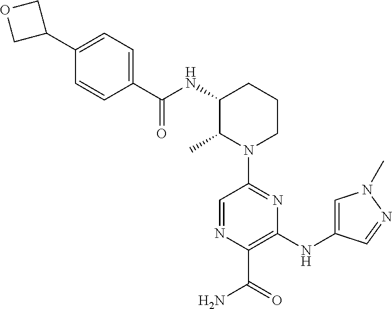 [SMILES: C[C@@H]1[C@H](NC(=O)c2ccc(C3COC3)cc2)CCCN1c1cnc(C(N)=O)c(Nc2cnn(C)c2)n1] | 3-(1-methyl-1H-pyrazol-4- ylamino)-5-((2R,3R)-2- methyl-3-(4-(oxetan-3- yl)benzamido)piperidin-1- yl)pyrazine-2-carboxamide |
| A-207 | 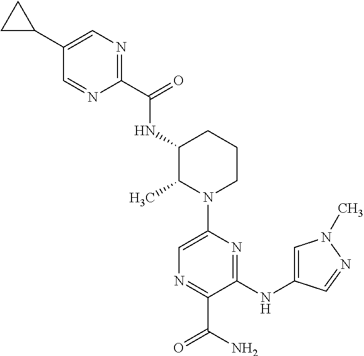 [SMILES: C[C@@H]1[C@H](NC(=O)c2ncc(C3CC3)cn2)CCCN1c1cnc(C(N)=O)c(Nc2cnn(C)c2)n1] | 5-[(2R,3R)-3-(5- cyclopropylpyrimidine- 2-amido)-2- methylpiperidin-1-yl]-3-[(1- methyl-1H-pyrazol-4- yl)amino]pyrazine-2- carboxamide |
| A-208 | 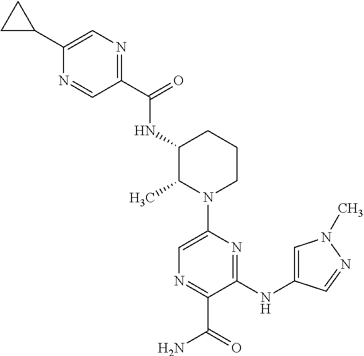 [SMILES: C[C@@H]1[C@H](NC(=O)c2cnc(C3CC3)cn2)CCCN1c1cnc(C(N)=O)c(Nc2cnn(C)c2)n1] | 5-[(2R,3R)-3-(5- cyclopropylpyrazine- 2-amido)-2- methylpiperidin-1-yl]-3-[(1- methyl-1H-pyrazol-4- yl)amino]pyrazine-2- carboxamide |
| A-209 | 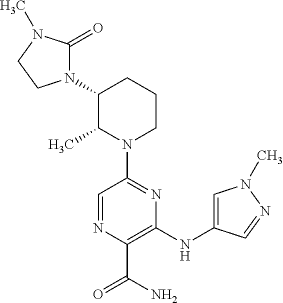 [SMILES: C[C@@H]1[C@H](N2CCN(C)C2=O)CCCN1c1cnc(C(N)=O)c(Nc2cnn(C)c2)n1] | 3-[(1-methyl-1H-pyrazol-4- yl)amino]-5-[(2R,3R)-2- methyl-3-(3-methyl-2- oxoimidazolidin-1- yl)piperidin-1-yl]pyrazine-2- carboxamide |
| A-210 | 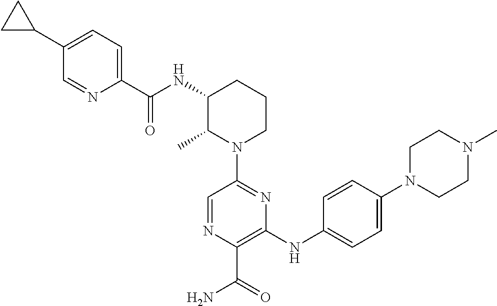 [SMILES: C[C@@H]1[C@H](NC(=O)c2ccc(C3CC3)cn2)CCCN1c1cnc(C(N)=O)c(Nc2ccc(N3CCN(C)CC3)cc2)n1] | 5-((2R,3R)-3-(5- cyclopropylpicolinamido)-2- methylpiperidin-1-yl)-3-(4-(4- methylpiperazin-1- yl)phenylamino)pyrazine-2- carboxamide |
| A-211 | 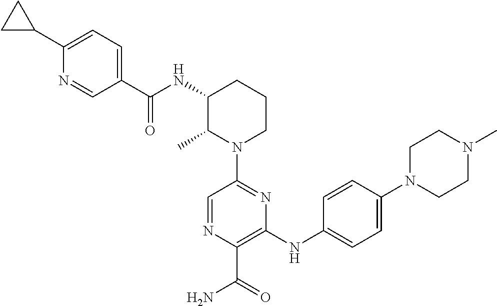 [SMILES: C[C@@H]1[C@H](NC(=O)c2ccc(C3CC3)nc2)CCCN1c1cnc(C(N)=O)c(Nc2ccc(N3CCN(C)CC3)cc2)n1] | 5-((2R,3R)-3-(6- cyclopropylnicotinamido)-2- methylpiperidin-1-yl)-3-(4-(4- methylpiperazin-1- yl)phenylamino)pyrazine-2- carboxamide |
| A-212 | 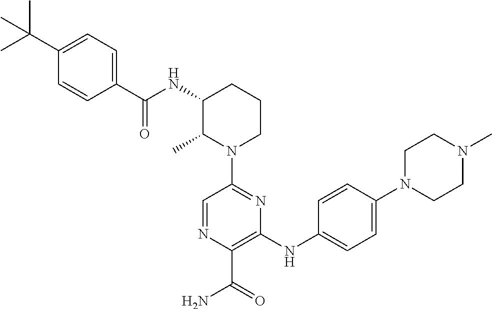 [SMILES: C[C@@H]1[C@H](NC(=O)c2ccc(C(C)(C)C)cc2)CCCN1c1cnc(C(N)=O)c(Nc2ccc(N3CCN(C)CC3)cc2)n1] | 5-((2R,3R)-3-(4-tert- butylbenzamido)-2- methylpiperidin-1-yl)-3-(4-(4- methylpiperazin-1- yl)phenylamino)pyrazine-2- carboxamide |
| A-213 | 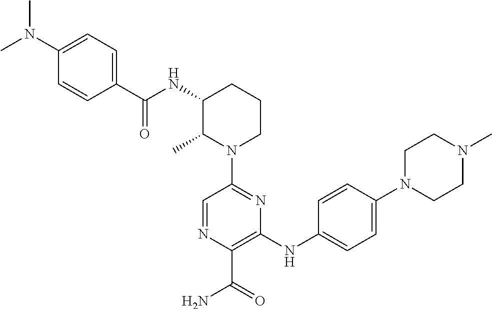 | 5-((2R,3R)-3-(4- (dimethylamino)benzamido)-2- methylpiperidin-1-yl)-3-(4-(4- methylpiperazin-1- yl)phenylamino)pyrazine-2- carboxamide |
| A-214 | 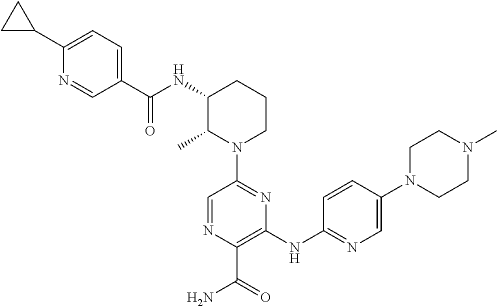 [SMILES: C[C@@H]1[C@H](NC(=O)c2ccc(C3CC3)nc2)CCCN1c1cnc(C(N)=O)c(Nc2ccc(N3CCN(C)CC3)cn2)n1] | 5-((2R,3R)-3-(6- cyclopropylnicotinamido)-2- methylpiperidin-1-yl)-3-(5-(4- methylpiperazin-1-yl)pyridin- 2-ylamino)pyrazine-2- carboxamide |
| A-215 | 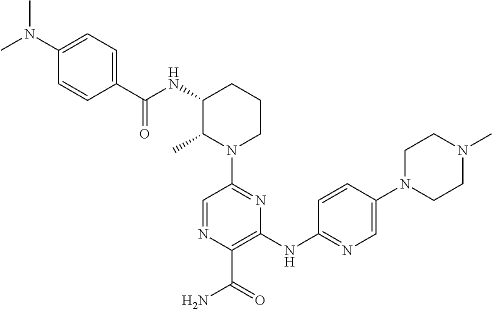 | 5-((2R,3R)-3-(4- (dimethylamino)benz- amido)-2-methyl- piperidin-1-yl)-3-(5-(4- methylpiperazin- 1-yl)pyridin-2- ylamino)pyrazine-2- carboxamide |
| A-216 | 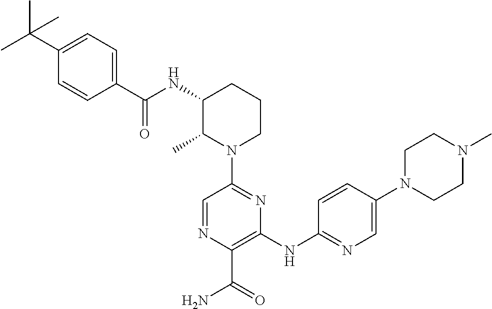 [SMILES: C[C@@H]1[C@H](NC(=O)c2ccc(C(C)(C)C)cc2)CCCN1c1cnc(C(N)=O)c(Nc2ccc(N3CCN(C)CC3)cn2)n1] | 5-((2R,3R)-3-(4-tert- butylbenzamido)-2- methylpiperidin-1-yl)-3-(5-(4- methylpiperazin-1-yl)pyridin- 2-ylamino)pyrazine-2- carboxamide |
| A-217 | 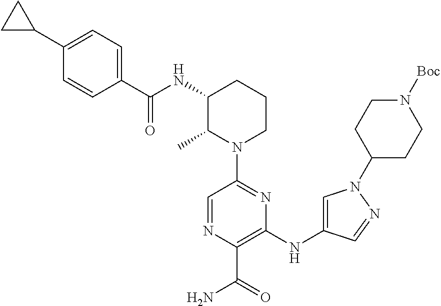 [SMILES: C[C@@H]1[C@H](NC(=O)c2ccc(C3CC3)cc2)CCCN1c1cnc(C(N)=O)c(Nc2cnn(C3CCN(C(=O)OC(C)(C)C)CC3)c2)n1] | tert-butyl 4-(4-(3-carbamoyl- 6-((2R,3R)-3-(4- cyclopropylbenzamido)-2- methylpiperidin-1-yl)pyrazin- 2-ylamino)-1H-pyrazol-1- yl)piperidin-1-carboxylate |
| A-218 | 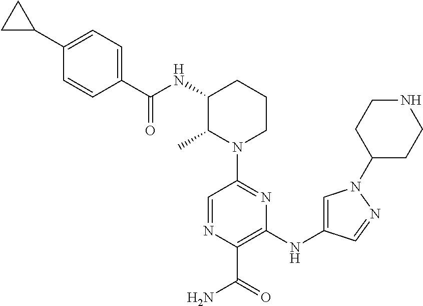 [SMILES: C[C@@H]1[C@H](NC(=O)c2ccc(C3CC3)cc2)CCCN1c1cnc(C(N)=O)c(Nc2cnn(C3CCNCC3)c2)n1] | 5-((2R,3R)-3-(4- cyclopropylbenzamido)-2- methylpiperidin-1-yl)-3-(1- (piperidin-4-yl)-1H-pyrazol-4- ylamino)pyrazine-2- carboxamide |
| A-219 | 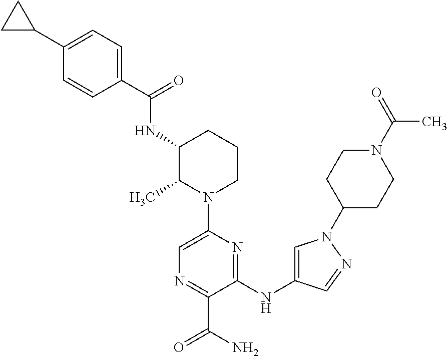 [SMILES: CC(=O)N1CCC(n2cc(Nc3nc(N4CCC[C@@H](NC(=O)c5ccc(C6CC6)cc5)[C@H]4C)cnc3C(N)=O)cn2)CC1] | 3-{[1-(1-acetylpiperidin-4-yl)- 1H-pyrazol-4- yl]amino}-5-[(2R,3)R-3-(4- cyclopropylbenzamido)-2- methylpiperidin-1-yl]pyrazine- 2-carboxamide |
| A-220 | 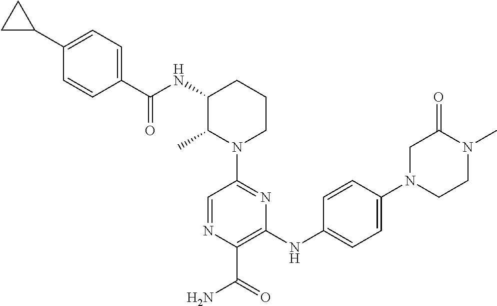 [SMILES: C[C@@H]1[C@H](NC(=O)c2ccc(C3CC3)cc2)CCCN1c1cnc(C(N)=O)c(Nc2ccc(N3CCN(C)C(=O)C3)cc2)n1] | 5-((2R,3R)-3-(4- cyclopropylbenzamido)-2- methylpiepridin-1-yl)-3-(4-(4- methyl-3-oxopiperazin-1- yl)phenylamino)pyrazine-2- carboxamide |
| A-221 | 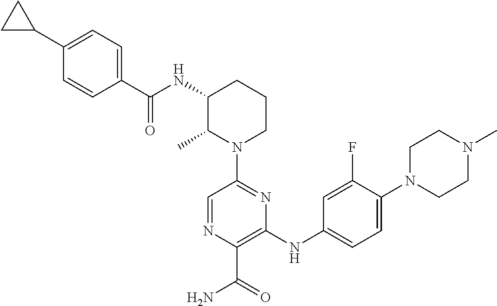 [SMILES: C[C@@H]1[C@H](NC(=O)c2ccc(C3CC3)cc2)CCCN1c1cnc(C(N)=O)c(Nc2ccc(N3CCN(C)CC3)c(F)c2)n1] | 5-((2R,3R)-3-(4- cyclopropylbenzamido)-2- methylpiperidin-1-yl)- 3-(3-fluoro- 4-(4-methylpiperazin-1- yl)phenylamino)pyrazine-2- carboxamide |
| A-222 | 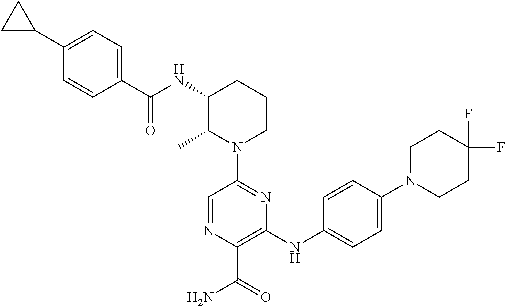 [SMILES: C[C@@H]1[C@H](NC(=O)c2ccc(C3CC3)cc2)CCCN1c1cnc(C(N)=O)c(Nc2ccc(N3CCC(F)(F)CC3)cc2)n1] | 5-((2R,3R)-3-(4- cyclopropylbenzamido)-2- methylpiperidin-1-yl)-3-(4- (4,4-difluoropiperidin-1- yl)phenylamino)pyrazine-2- carboxamide |
| A-223 | 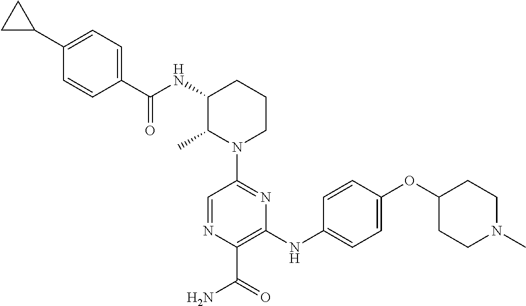 [SMILES: C[C@@H]1[C@H](NC(=O)c2ccc(C3CC3)cc2)CCCN1c1cnc(C(N)=O)c(Nc2ccc(OC3CCN(C)CC3)cc2)n1] | 5-((2R,3R)-3-(4- cyclopropylbenzamido)-2- methylpiperidin-1-yl)-3-(4-(1- methylpiperidin-4- yloxy)phenylamino)pyrazine- 2-carboxamide |
| A-224 | 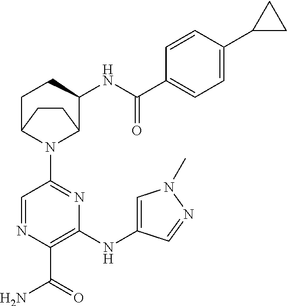 [SMILES: Cn1cc(Nc2nc(N3C4CCC3[C@H](NC(=O)c3ccc(C5CC5)cc3)CC4)cnc2C(N)=O)cn1] | 5-((2R)-2-(4- cyclopropylbenzamido)-8- azabicyclo[3.2.1]octan- 8-yl)-3-(1- methyl-1H-pyrazol-4- ylamino)pyrazine-2- carboxamide |
| A-225 | 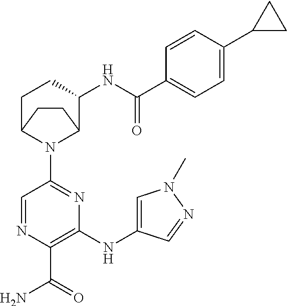 [SMILES: Cn1cc(Nc2nc(N3C4CCC3[C@@H](NC(=O)c3ccc(C5CC5)cc3)CC4)cnc2C(N)=O)cn1] | 5-((2S)-2-(4- cyclopropylbenzamido)-8- azabicyclo[3.2.1]octan- 8-yl)-3-(1- methyl-1H-pyrazol-4- ylamino)pyrazine-2- carboxamide |
| A-226 | 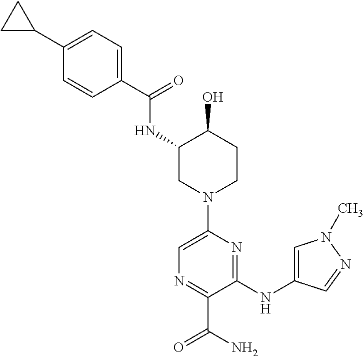 [SMILES: Cn1cc(Nc2nc(N3CC[C@H](O)[C@@H](NC(=O)c4ccc(C5CC5)cc4)C3)cnc2C(N)=O)cn1] | 5-[(3S,4S)-3-(4- cyclopropylbenzamido)-4- hydroxypiperidin-1-yl]-3-[(1- methyl-1H-pyrazol-4- yl)amino]pyrazine-2- carboxamide |
| A-227 | 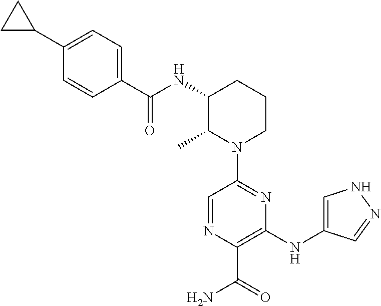 [SMILES: C[C@@H]1[C@H](NC(=O)c2ccc(C3CC3)cc2)CCCN1c1cnc(C(N)=O)c(Nc2cn[nH]c2)n1] | 3-(1H-pyrazol-4-ylamino)-5- ((2R,3R)-3-(4- cyclopropylbenzamido)-2- methylpiperidin-1-yl)pyrazine- 2-carboxamide |
| A-228 | 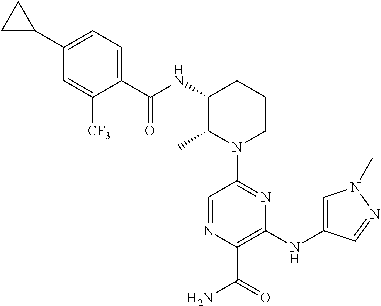 [SMILES: C[C@@H]1[C@H](NC(=O)c2ccc(C3CC3)cc2C(F)(F)F)CCCN1c1cnc(C(N)=O)c(Nc2cnn(C)c2)n1] | 5-((2R,3R)-3-(4- cyclopropyl-2-(tri- fluoromethyl)benzamido)-2- methylpiperidin-1-yl)- 3-(1-methyl-1H-pyrazol- 4-ylamino)pyrazine-2- carboxamide |
| A-229 | 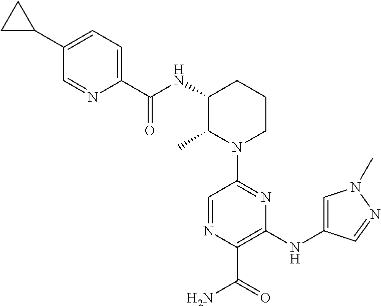 [SMILES: C[C@@H]1[C@H](NC(=O)c2ccc(C3CC3)cn2)CCCN1c1cnc(C(N)=O)c(Nc2cnn(C)c2)n1] | 5-((2R,3R)-3-(5- cyclopropylpicolinamido)-2- methylpiperidin- 1-yl)-3-(1-methyl- 1H-pyrazol-4- ylamino)pyrazine- 2-carboxamide |
| A-230 | 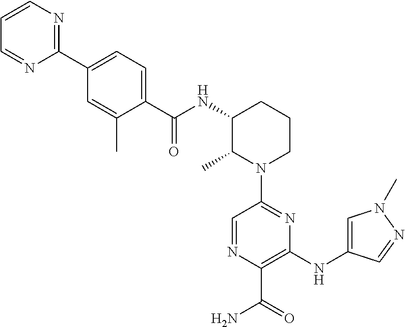 [SMILES: Cc1cc(-c2ncccn2)ccc1C(=O)N[C@@H]1CCCN(c2cnc(C(N)=O)c(Nc3cnn(C)c3)n2)[C@@H]1C] | 3-(1-methyl-1H-pyrazol-4- ylamino)-5-((2R,3R)-2- methyl-3-(4-(pyrimidin-2- yl)benzamido)piperidin-1- yl)pyrazine-2-carboxamide |
| A-231 | 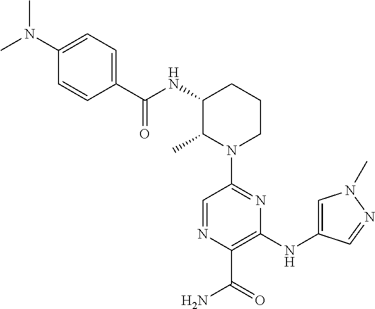 [SMILES: C[C@@H]1[C@H](NC(=O)c2ccc(N(C)C)cc2)CCCN1c1cnc(C(N)=O)c(Nc2cnn(C)c2)n1] | 5-((2R,3R)-3-(4- (dimethyalmino)benzamido)- 2-methylpiperidin- 1-yl)-3-(1-methyl-1H-pyrazol- 4-ylamino)pyrazine-2- carboxamide |
| A-232 | 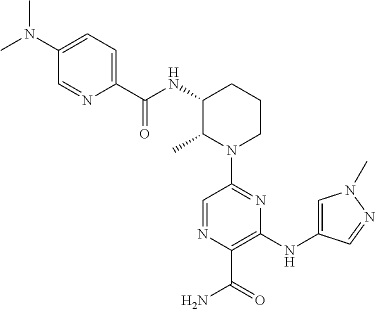 [SMILES: C[C@@H]1[C@H](NC(=O)c2ccc(N(C)C)cn2)CCCN1c1cnc(C(N)=O)c(Nc2cnn(C)c2)n1] | 5-((2R,3R)-3-(5- (dimethylamino)picolin- amido)-2-methylpiperidin- 1-yl)-3-(1-methyl-1H- pyrazol-4-ylamino)pyrazine- 2-carboxamide |
| A-233 | 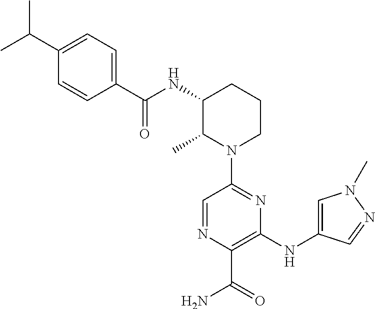 [SMILES: CC(C)c1ccc(C(=O)N[C@@H]2CCCN(c3cnc(C(N)=O)c(Nc4cnn(C)c4)n3)[C@@H]2C)cc1] | 5-((2R,3R)-3-(4- isopropylbenzamido)-2- methylpiperidin-1-yl)- 3-(1-methyl-1H-pyrazol- 4-ylamino)pyrazine-2- carboxamide |

### Example B-1: 3-[(3-methyl-1,2-thiazol-5-yl)amino]-5-{[(1r,4r)-4-[4-(dimethylamino) benzamido]cyclohexyl]amino}pyrazine-2-carboxamide (1)

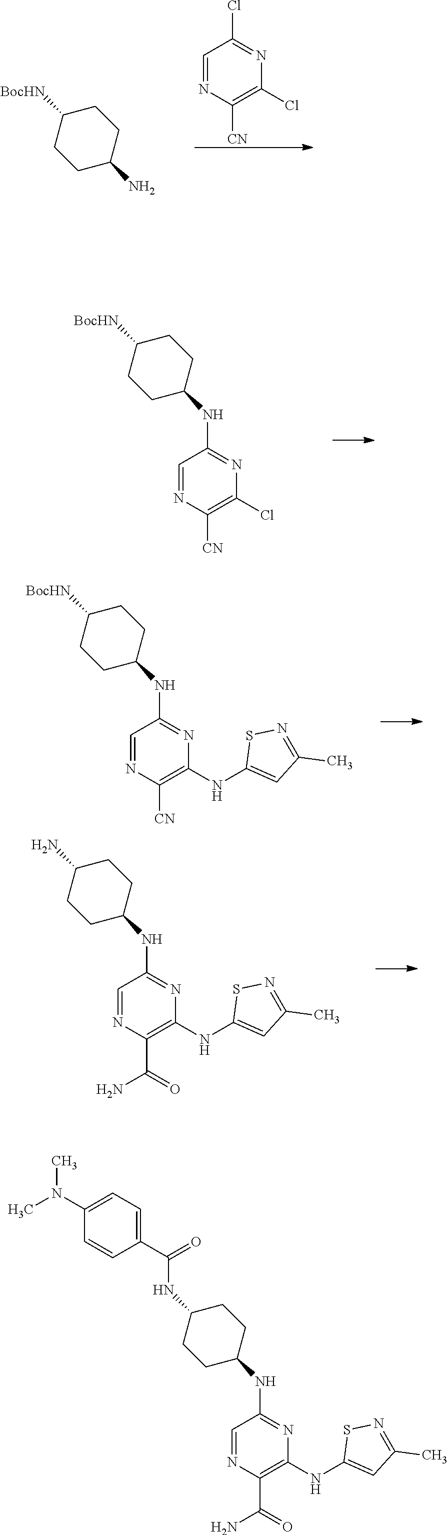

Tert-butyl N-[(1R,4R)-4-aminocyclohexyl]carbamate (2.0 g, 9.32 mmol) and 3,5-dichloropyrazine-2-carbonitrile (1.62 g, 9.32 mmol) were suspended in 40 ml of DMF, DIPEA (3.24 ml, 18.64 mmol) was added and the reaction was left stirring 2h at room temperature. The mixture was concentrated, ethyl acetate and water were added, the phases were separated and the organic one was dried over Na₂SO₄, filtered and concentrated. The residue was triturated with Et₂O to give 2.61 g of tert-butyl N-{4-[(6-chloro-5-cyanopyrazin-2-yl)amino]cyclohexyl}carbamate (2.61 g, 79.6% yield).

Tert-butyl N-({4-[(6-chloro-5-cyanopyrazin-2-yl)amino]cyclohexyl}carbamate (500 mg, 1.4 mmol), 5-amino-3-methyl-isothiazole hydrochloride (891.0 mg, 5.92 mmol), Cs₂CO₃ (1.37 g, 4.2 mmol), BINAP(±) (184 mg, 0.296 mmol) and Pd(OAc)₂ (66 mg, 0.296 mmol) were added. The mixture was degassed bubbling N₂ for 10 minutes and then refluxed overnight. The mixture was concentrated and the residue was purified flash chromatography (silica) eluting with ethyl acetate in cyclohexane from 30 to 50% to give tert-butyl N-[4-({5-cyano-6-[(3-methyl-1,2-thiazol-5-yl)amino]pyrazin-2-yl}amino)cyclohexyl]carbamate (122.0 mg, 0.284 mmol). This compound was dissolved in 3 ml of TFA and 0.1 ml of H₂SO₄ was added. The reaction was left stirring 8 hours at room temperature. Na₂CO₃ saturated aqueous solution was added and the mixture was concentrated in vacuo. The residue was dissolved in MeOH and passed through SCX cartridge eluting with NH₃ 7 N in MeOH solution. The filtrate was concentrated in vacuo to give methyl 5-[(4-aminocyclohexyl)amino]-3-[(3-methyl-1,2-thiazol-5-yl)amino]pyrazine-2-carboxamide (69 mg, 69.9% yield).

4-Dimethylaminobenzoyl chloride (43.63 mg, 0.237 mmol) and DIPEA (0.104 ml, 0.594 mmol) were added to a solution of 5-[(4-aminocyclohexyl)amino]-3-[(3-methyl-1,2-thiazol-5-yl)amino]pyrazine-2-carboxamide (69.0 mg, 0.198 mmol) in 3 ml of DMF and the reaction was stirred at room temperature 8 hours. The mixture was concentrated and the residue purified flash chromatography (silica) eluting with MeOH in DCM from 0 to 5% to give 5-({4-[4-(dimethylamino)benzamido]cyclohexyl}amino)-3-[(3-methyl-1,2-thiazol-5-yl)amino]pyrazine-2-carboxamide (36 mg, 0.072 mmol, 35.6% yield). MS found for C24H30N8O2S as (M+H)⁺ 495.0. ¹H NMR (500 MHz, DMSO) δ 12.28 (br. s, 1H), 8.02 (d, J=7.83 Hz, 2H), 7.84-7.70 (m, 3H), 7.47-7.39 (m, 2H), 6.69 (d, J=9.29 Hz, 2H), 6.84 (s, 1H), 4.06-3.95 (m, 1H), 3.92-3.81 (m, 1H), 2.96 (s, 6H), 2.29 (s, 3H), 2.15 (d, J=11.00 Hz, 2H), 1.93 (d, J=11.00 Hz, 2H), 1.63 (q, J=14.00 Hz, 2H), 1.40 (q, J=14.00 Hz, 2H).

### Example 2: 3-[(quinolin-6-yl)amino]-5-{[(1r,4r)-4-benzamidocyclohexyl]amino}pyrazine-2-carboxamide (2)

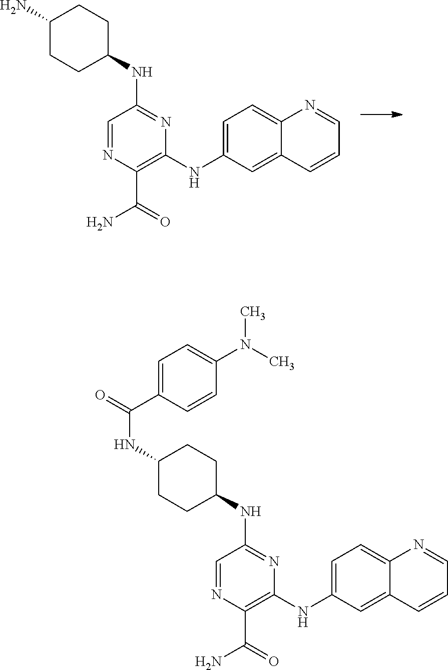 [SMILES: NC(=O)c1ncc(N[C@H]2CC[C@H](N)CC2)nc1Nc1ccc2ncccc2c1 ; CN(C)c1ccc(C(=O)N[C@H]2CC[C@H](Nc3cnc(C(N)=O)c(Nc4ccc5ncccc5c4)n3)CC2)cc1]

In a similar manner as described in Example B-1, 3-[(quinolin-6-yl)amino]-5-{[(1r,4r)-4-benzamidocyclohexyl]amino}pyrazine-2-carboxamide (2) was prepared using 4-dimethylaminobenzoyl chloride. MS found for C27H27N7O2 as (M+H)⁺ 525.2. ¹H NMR (500 MHz, DMSO) δ 11.81 (s, 1H), 8.74 (dd, J=4.12, 1.65 Hz, 1H), 8.34 (br. s., 1H), 8.21 (d, J=7.96 Hz, 1H), 8.02-7.94 (m, 3H), 7.82-7.71 (m, 4H), 7.48 (dd, J=8.20, 4.10 Hz, 1H), 7.41 (s, 1H), 7.34 (br. s., 1H), 6.71 (d, J=8.78 Hz, 2H), 3.90-3.72 (m, 2H), 2.97 (s, 6H), 2.21-2.10 (m, 2H), 1.99-1.87 (m, 2H), 1.64-1.51 (m, 2H), 1.48-1.34 (m, 2H).

### Example B-3: 5-({4-[(dimethylcarbamoyl)amino]cyclohexyl}amino)-3-[(3-methyl-1,2-thiazol-5-yl)amino]pyrazine-2-carboxamide (3)

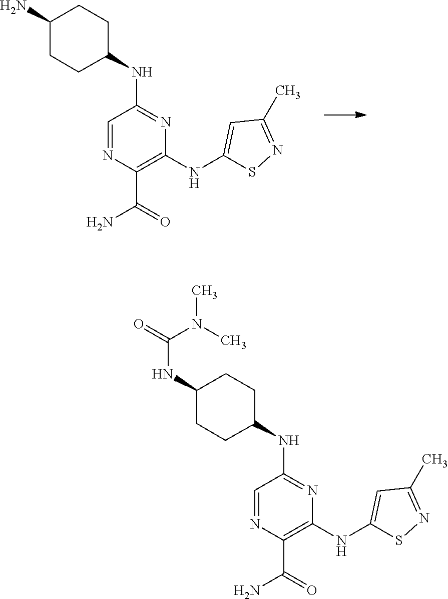

In a similar manner as described in Example 1, 5-({4-[(dimethylcarbamoyl)amino]cyclohexyl}amino)-3-[(3-methyl-1,2-thiazol-5-yl)amino]pyrazine-2-carboxamide (3) was prepared using N,N-dimethylcarbamoyl chloride. MS found for C18H26N8O2S as (M+H)⁺ 419.0. ¹H NMR (500 MHz, DMSO) δ 12.23 (s, 1H), 7.97-7.84 (m, 1H), 7.79 (br. s., 1H), 7.54 (s, 1H), 7.42 (br. s., 1H), 6.82 (s, 1H), 5.83-5.67 (m, 1H), 4.22-4.04 (m, 1H), 1.94-1.51 (m, 8H), 3.67-3.55 (m, 1H), 2.80 (s, 6H), 2.28 (s, 3H).

### Example B-4: 5-[(4-benzamidocyclohexyl)amino]-3-[(3-methyl-1,2-thiazol-5-yl)amino]pyrazine-2-carboxamide (4)

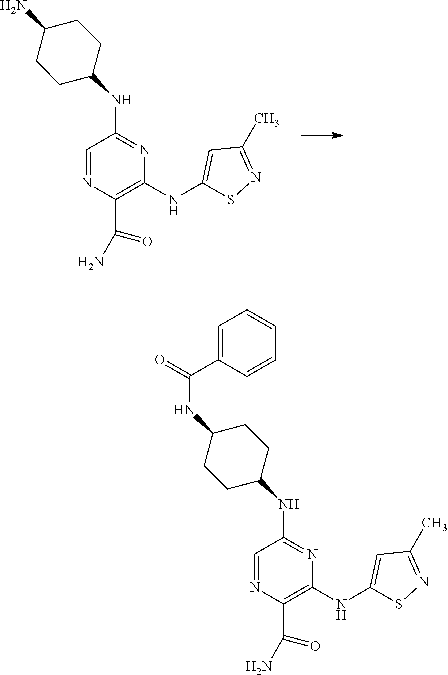

In a similar manner as described in Example B-1, 5-[(4-benzamidocyclohexyl)amino]-3-[(3-methyl-1,2-thiazol-5-yl)amino]pyrazine-2-carboxamide (4) was prepared using benzoyl chloride. MS found for C22H25N7O2S as (M+H)⁺ 451.9. ¹H NMR (500 MHz, DMSO) δ 12.24 (s, 1H), 8.21 (d, J=6.31 Hz, 1H), 8.02-7.88 (m, 1H), 7.88-7.82 (m, 1H), 7.79 (br. s., 1H), 7.56 (s, 1H), 7.54-7.49 (m, 1H), 7.48-7.39 (m, 3H), 6.83 (s, 1H), 4.18 (br. s., 1H), 3.96 (br. s., 1H), 2.29 (s, 3H), 2.01-1.66 (m, 8H).

### Example B-5: 5-({4-[4-(dimethylamino)benzamido]cyclohexyl}amino)-3-[(3-methyl-1,2-thiazol-5-yl)amino]pyrazine-2-carboxamide (5)

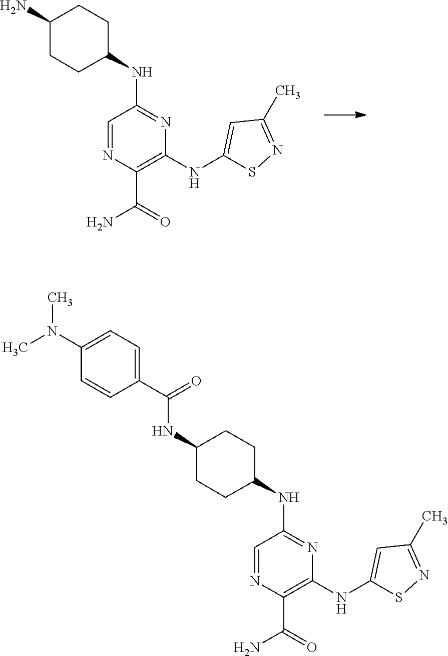

In a similar manner as described in Example B-1, 5-({4-[4-(dimethylamino)benzamido]cyclohexyl}amino)-3-[(3-methyl-1,2-thiazol-5-yl)amino]pyrazine-2-carboxamide (5) was prepared using 4-dimethylaminobenzoyl chloride. MS found for C24H30N8O2S as (M+H)⁺ 495.0. ¹H NMR (500 MHz, DMSO) δ 12.24 (s, 1H), 8.00-7.90 (m, 1H), 7.82-7.71 (m, 4H), 7.56 (s, 1H), 7.43 (br. s, 1H), 6.83 (s, 1H), 6.69 (d, J=8.80 Hz, 2H), 4.26-4.12 (m, 1H), 4.00-3.85 (m, 1H), 2.96 (s, 6H), 2.29 (s, 3H), 2.00-1.64 (m, 8H).

### Example B-6: 5-(methyl((1r,4r)-4-(N-methylacrylamido)cyclohexyl)amino)-3-(3-methylisothiazol-5-ylamino)pyrazine-2-carboxamide (6)

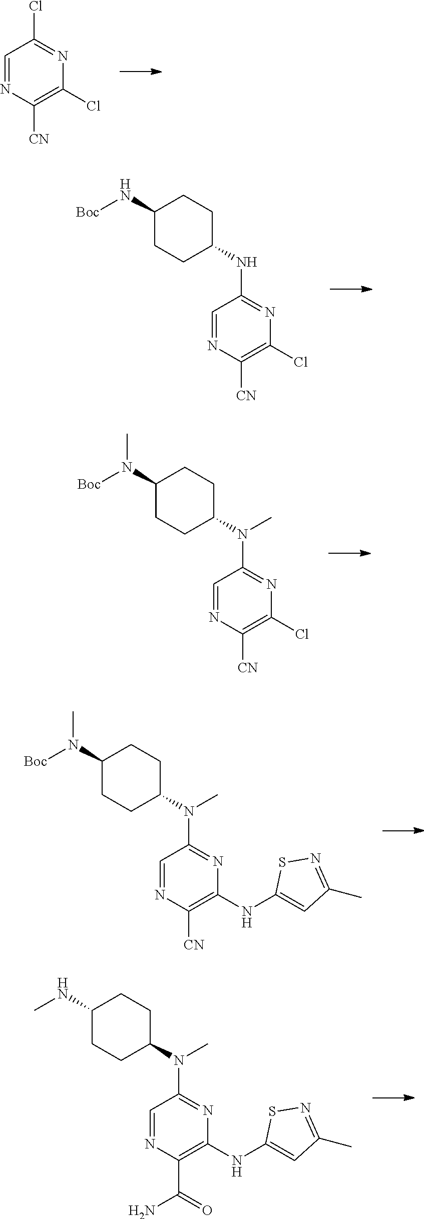

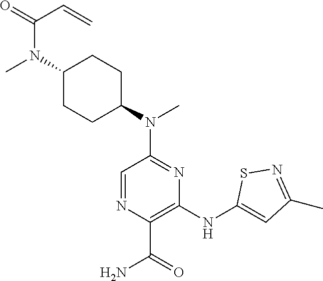

To a solution of 3,5-dichloropyrazine-2-carbonitrile (1.00 g, 5.75 mmol) in DMF (20 mL) were added N-Boc-trans-1,4-diaminocyclohexane (1.35 g, 6.32 mmol) and then DIPEA (1.5 mL, 8.62 mmol) in a dropwise manner. The mixture was stirred at room temperature for 5 hrs. To it was poured water. The mixture was rapidly stirred for 30 min, and the solid was isolated by filtration. It was washed with water and dried in vacuo as tert-butyl (1r,4r)-4-(6-chloro-5-cyanopyrazin-2-ylamino)cyclohexylcarbamate (1.70 g, 84%) in excellent purity.

Tert-butyl (1r,4r)-4-(6-chloro-5-cyanopyrazin-2-ylamino)cyclohexylcarbamate (350 mg, 1.0 mmol) was dissolved in dry DMSO. To it was added NaH (60% in mineral oil, 320 mg, 8.0 mmol). The mixture was stirred for 10 min, and then iodomethane (620 μL, 10 mmol) was added. After 1.5 hr, extra NaH (160 mg) and iodomethane (310 μL) were added. The mixture was stirred at RT for overnight. It was diluted with EtOAc, washed with water ×3, dried, concentrated, and subjected to silica flash column to isolate tert-butyl (1r,4r)-4-((6-chloro-5-cyanopyrazin-2-yl)(methyl)amino)cyclohexyl(methyl)carbamate using 0 to 20% EtOAc in DCM.

A mixture of tert-butyl (1r,4r)-4-((6-chloro-5-cyanopyrazin-2-yl)(methyl)amino)cyclohexyl(methyl)carbamate (150 mg, 0.40 mmol), 5-amino-3-methylisothiazole hydrochloride (180 mg, 1.20 mmol), Pd(OAc)₂ (27 mg, 0.12 mmol), BINAP (75 mg, 0.12 mmol), fine powder Cs₂CO₃ (1.30 g, 4.0 mmol) in dioxane (25 mL, with 40 μL water) was degassed with a nitrogen stream for 5 min. The mixture was stirred in a nitrogen atmosphere at 115° C. for 2 hrs. The mixture was cooled to room temperature, diluted with ethyl acetate, filtered through a disposable ChemGlass filter (cat# OP-6602-12), and concentrated in vacuo. The residue was purified by flash chromatography with 10 to 75% ethyl acetate in hexane to give tert-butyl (1r,4r)-4-((5-cyano-6-(3-methylisothiazol-5-ylamino)pyrazin-2-yl)(methyl)amino)cyclohexyl(methyl)carbamate. It was treated with 6 mL TFA and 1 mL concentrated H₂SO₄ at 80° C. for 40 min. The mixture was cooled to RT, diluted water, and subjected to reverse phase preparative HPLC, using 5.0 mM HCl and neat acetonitrile as mobile phases, to isolate 5-(methyl((1r,4r)-4-(methylamino)cyclohexyl)amino)-3-(3-methylisothiazol-5-ylamino)pyrazine-2-carboxamide as HCl salt (131 mg). MS found for C17H25N7OS as (M+H)⁺ 376.2 and (M−H)⁻ 374.1.

To a solution of 5-(methyl((1r,4r)-4-(methylamino)cyclohexyl)amino)-3-(3-methylisothiazol-5-ylamino)pyrazine-2-carboxamide (HCl salt, 50 mg, 0.12 mmol) in 2 mL NMP in ice bath were added DIPEA (110 μL, 0.60 mmol) and 2 minutes later acryloyl chloride (20 μl, 0.24 mmol). The mixture was stirred for 5 minutes, then quenched with 0.2 mL TFA and diluted with 2 mL water. The mixture was directly subjected to reverse phase preparative HPLC, using 0.1% formic acid in water and neat acetonitrile as mobile phases, to give the title compound 5-(methyl((1r,4r)-4-(N-methylacrylamido)cyclohexyl)amino)-3-(3-methylisothiazol-5-ylamino)pyrazine-2-carboxamide as formic acid salt (31 mg). MS found for C20H27N7O2S as (M+H)⁺ 430.5 and (M−H)⁻ 428.2.

Compounds in Table 2 were prepared according to procedures similar to or same as those described herein or methods known in the art.

TABLE 2
Compounds of Formula (B-I)

| Cmpd. | | | |
|---|---|---|---|
| No. | Compound Structure | Compound Name | MS |
| B-7 | 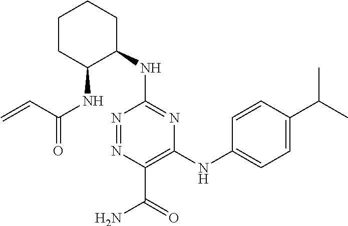 [SMILES: C=CC(=O)N[C@H]1CCCC[C@H]1Nc1nnc(C(N)=O)c(Nc2ccc(C(C)C)cc2)n1] | 3-((1R,2S)-2-acrylamido- cyclohexylamino)-5-(4- isopropylphenylamino)- 1,2,4-triazine-6- carboxamide | M + H = 424.4 M − H = 422.1 |
| B-8 | 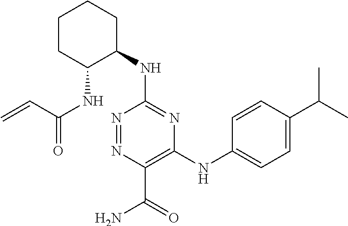 [SMILES: C=CC(=O)N[C@@H]1CCCC[C@H]1Nc1nnc(C(N)=O)c(Nc2ccc(C(C)C)cc2)n1] | 3-((1R,2R)-2-acrylamido- cyclohexylamino)-5-(4- isopropylphenylamino)- 1,2,4-triazine-6- carboxamide | M + H = 424.4 M − H = 422.1 |
| B-9 | 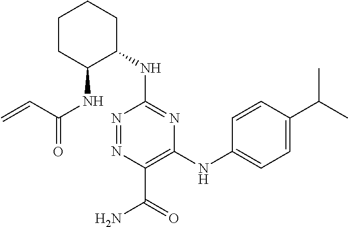 [SMILES: C=CC(=O)N[C@H]1CCCC[C@@H]1Nc1nnc(C(N)=O)c(Nc2ccc(C(C)C)cc2)n1] | 3-((1S,2S)-2-acrylamido- cyclohexylamino)-5-(4- isopropylphenylamino)- 1,2,4-triazine-6- carboxamide | M + H = 424.4 M − H = 422.1 |
| B-10 | 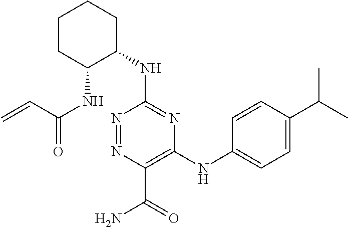 [SMILES: C=CC(=O)N[C@@H]1CCCC[C@@H]1Nc1nnc(C(N)=O)c(Nc2ccc(C(C)C)cc2)n1] | 3-((1S,2R)-2-acrylamido- cyclohexylamino)-5-(4- isopropylphenylamino)- 1,2,4-triazine-6- carboxamide | M + H = 424.4 M − H = 422.2 |
| B-11 | 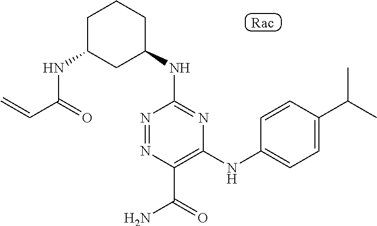 [SMILES: C=CC(=O)N[C@@H]1CCC[C@@H](Nc2nnc(C(N)=O)c(Nc3ccc(C(C)C)cc3)n2)C1 ; "Rac"] | 3-((1R,3R)-3-acrylamido- cyclohexylamino)-5-(4- isopropylphenylamino)- 1,2,4-triazine-6- carboxamide | M + H = 424.4 M − H = 422.1 |
| B-12 | 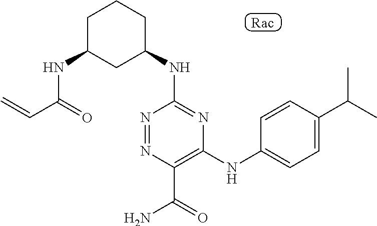 [SMILES: C=CC(=O)N[C@H]1CCC[C@@H](Nc2nnc(C(N)=O)c(Nc3ccc(C(C)C)cc3)n2)C1 ; "Rac"] | 3-((1R,3S)-3-acrylamido- cyclohexylamino)-5-(4- isopropylphenylamino)- 1,2,4-triazine-6- carboxamide | M + H = 424.3 M − H = 422.1 |
| B-13 | 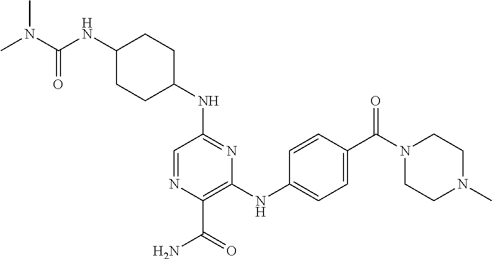 | 5-(4-(3,3- dimethylureido)cyclo- hexylamino)-3-(4-(4- methylpiperazine-1- carbonyl)phenylamino) pyrazine-2-carboxamide | |
| B-14 | 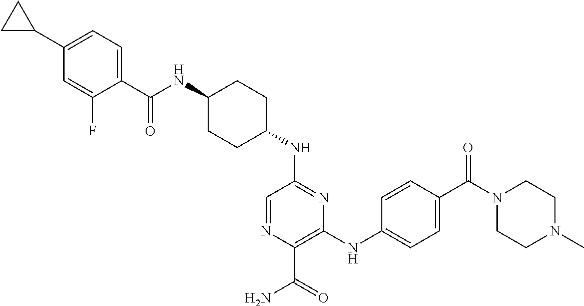 [SMILES: CN1CCN(C(=O)c2ccc(Nc3nc(N[C@H]4CC[C@H](NC(=O)c5ccc(C6CC6)cc5F)CC4)cnc3C(N)=O)cc2)CC1] | 5-((1r,4r)-4-(4-cyclo- propyl-2-fluoro- benzamido)cyclohexyl- amino)-3-(4-(4- methylpiperazine-1- carbonyl)phenylamino) pyrazine-2-carboxamide | |
| B-15 | 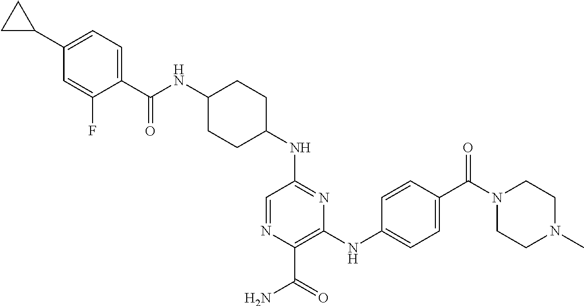 [SMILES: CN1CCN(C(=O)c2ccc(Nc3nc(NC4CCC(NC(=O)c5ccc(C6CC6)cc5F)CC4)cnc3C(N)=O)cc2)CC1] | 5-(4-(4-cyclopropyl- 2-fluoro- benzamido)cyclohexyl- amino)-3-(4-(4- methylpiperazine-1- carbonyl)phenylamino) pyrazine-2-carboxamide | |
| B-16 | 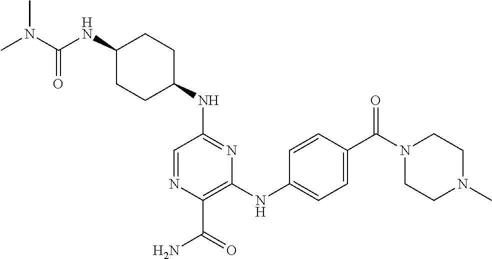 | 5-((1s,4s)-4-(3,3-dimethyl- ureido)cyclohexyl- amino)-3-(4-(4- methylpiperazine-1- carbonyl)phenylamino) pyrazine-2-carboxamide | |

Compounds in Table 3 may be prepared according to the procedures described herein or methods known in the art.

TABLE 3
Additional Compounds of Formula (B-I)

| Cmpd. No. | Compound Structure | Compound Name |
|---|---|---|
| B-17 | 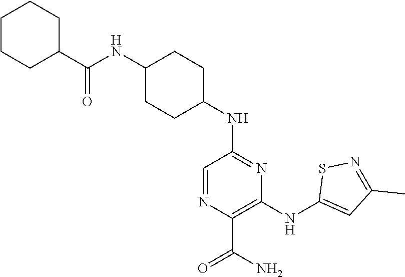 [SMILES: Cc1cc(Nc2nc(NC3CCC(NC(=O)C4CCCCC4)CC3)cnc2C(N)=O)sn1] | 5-(4- (cyclohexanecarboxamido)cyclohexyl- amino)-3-(3-methylisothiazol-5- ylamino)pyrazine-2-carboxamide |
| B-18 | 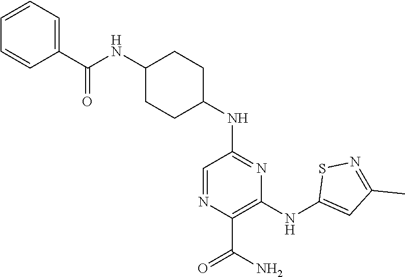 [SMILES: Cc1cc(Nc2nc(NC3CCC(NC(=O)c4ccccc4)CC3)cnc2C(N)=O)sn1] | 5-(4-benzamidocyclohexylamino)-3-(3- methylisothiazol-5-ylamino)pyrazine- 2-carboxamide |
| B-19 | 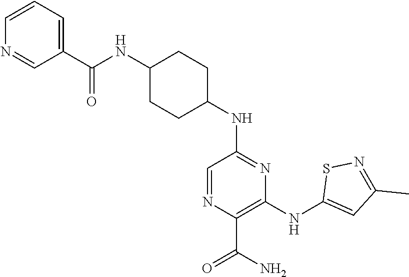 [SMILES: Cc1cc(Nc2nc(NC3CCC(NC(=O)c4cccnc4)CC3)cnc2C(N)=O)sn1] | 3-(3-methylisothiazol-5-ylamino)-5-(4- (nicotinamido)cyclohexyl- amino)pyrazine-2-carboxamide |
| B-20 | 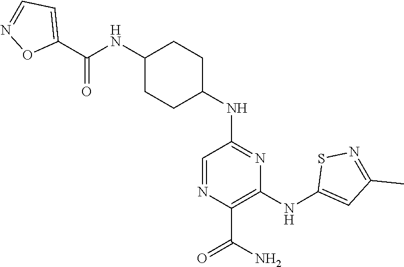 [SMILES: Cc1cc(Nc2nc(NC3CCC(NC(=O)c4ccno4)CC3)cnc2C(N)=O)sn1] | N-(4-(5-carbamoyl-6-(3- methylisothiazol-5-ylamino)pyrazin- 2-ylamino)cyclohexyl)isoxazole-5- carboxamide |
| B-21 | 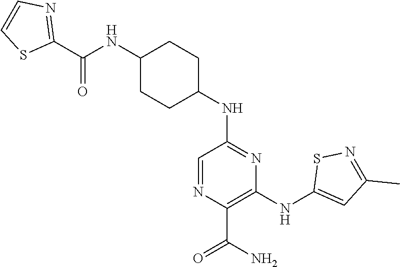 [SMILES: Cc1cc(Nc2nc(NC3CCC(NC(=O)c4nccs4)CC3)cnc2C(N)=O)sn1] | N-(4-(5-carbamoyl-6-(3- methylisothiazol-5-ylamino)pyrazin-2- ylamino)cyclohexyl)thiazole-2- carboxamide |
| B-22 | 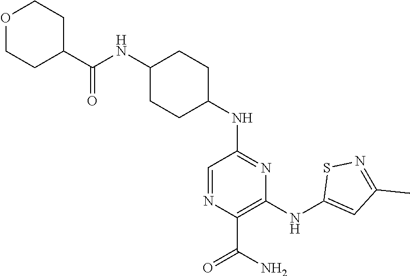 [SMILES: Cc1cc(Nc2nc(NC3CCC(NC(=O)C4CCOCC4)CC3)cnc2C(N)=O)sn1] | 3-(3-methylisothiazol-5-ylamino)-5-(4- (tetrahydro-2H-pyran-4- carboxamido)cyclohexyl- amino)pyrazine-2-carboxamide |
| B-23 | 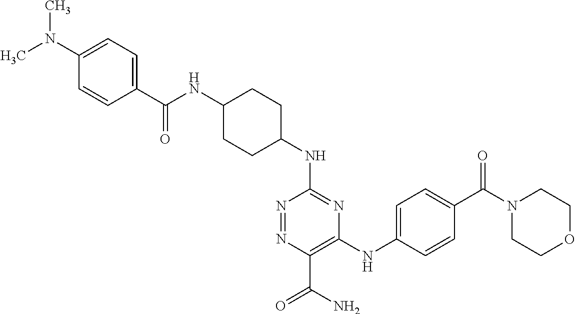 [SMILES: CN(C)c1ccc(C(=O)NC2CCC(Nc3nnc(C(N)=O)c(Nc4ccc(C(=O)N5CCOCC5)cc4)n3)CC2)cc1] | 3-(4-(4- (dimethylamino)benzamido)cyclohexyl- amino)-5-(4-(morpholine-4- carbonyl)phenylamino)-1,2,4-triazine- 6-carboxamide |
| B-24 | 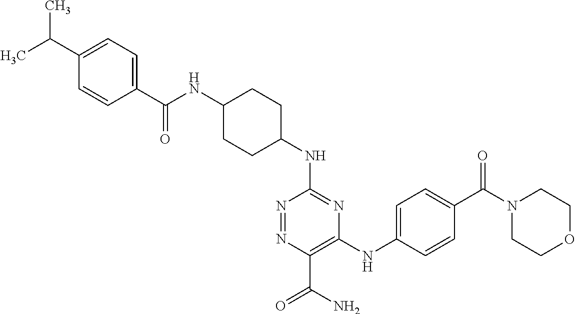 [SMILES: CC(C)c1ccc(C(=O)NC2CCC(Nc3nnc(C(N)=O)c(Nc4ccc(C(=O)N5CCOCC5)cc4)n3)CC2)cc1] | 3-(4-(4- isopropylbenzamido)cyclohexylamino)- 5-(4-(morpholine-4- carbonyl)phenylamino)-1,2,4-triazine- 6-carboxamide |
| B-25 | 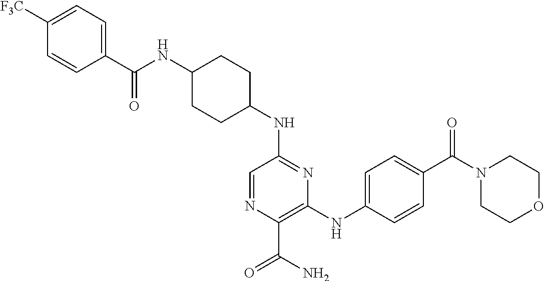 [SMILES: NC(=O)c1ncc(NC2CCC(NC(=O)c3ccc(C(F)(F)F)cc3)CC2)nc1Nc1ccc(C(=O)N2CCOCC2)cc1] | 3-(4-(morpholine-4- carbonyl)phenylamino)-5-(4-(4- (trifluoromethyl)benzamido)cyclohexyl- amino)pyrazine-2-carboxamide |
| B-26 | 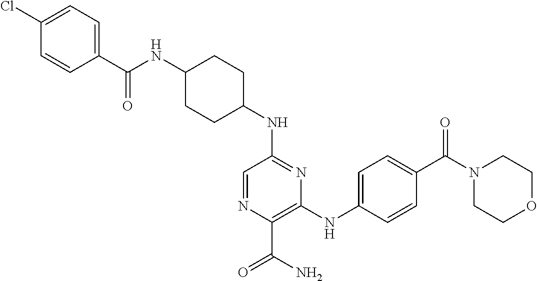 [SMILES: NC(=O)c1ncc(NC2CCC(NC(=O)c3ccc(Cl)cc3)CC2)nc1Nc1ccc(C(=O)N2CCOCC2)cc1] | 5-(4-(4- chlorobenzamido)cyclohexylamino)-3- (4-(morpholine-4- carbonyl)phenylamino)pyrazine-2- carboxamide |
| B-27 | 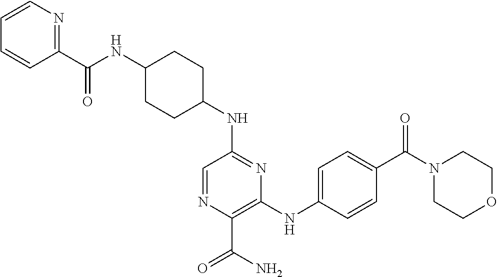 [SMILES: NC(=O)c1ncc(NC2CCC(NC(=O)c3ccccn3)CC2)nc1Nc1ccc(C(=O)N2CCOCC2)cc1] | 3-(4-(morpholine-4- carbonyl)phenylamino)-5-(4- (picolinamido)cyclohexyl- amino)pyrazine-2-carboxamide |
| B-28 | 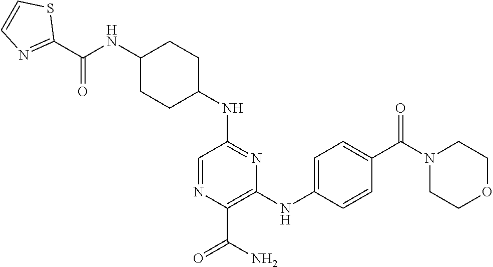 [SMILES: NC(=O)c1ncc(NC2CCC(NC(=O)c3nccs3)CC2)nc1Nc1ccc(C(=O)N2CCOCC2)cc1] | N-(4-(5-carbamoyl-6-(4-(morpholine-4- carbonyl)phenylamino)pyrazin-2- ylamino)cyclohexyl)thiazole-2- carboxamide |
| B-29 | 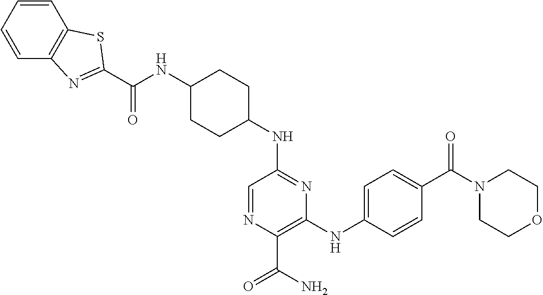 [SMILES: NC(=O)c1ncc(NC2CCC(NC(=O)c3nc4ccccc4s3)CC2)nc1Nc1ccc(C(=O)N2CCOCC2)cc1] | N-(4-(5-carbamoyl-6-(4-(morpholine-4- carbonyl)phenylamino)pyrazin-2- ylamino)cyclohexyl)benzo[d]thiazole- 2-carboxamide |
| B-30 | 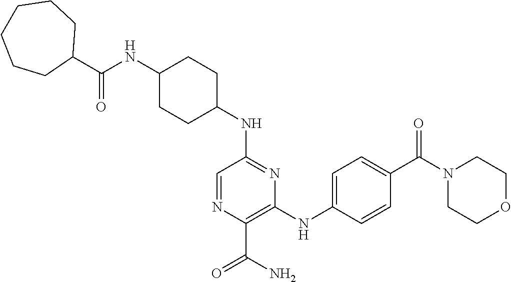 [SMILES: NC(=O)c1ncc(NC2CCC(NC(=O)C3CCCCCC3)CC2)nc1Nc1ccc(C(=O)N2CCOCC2)cc1] | 5-(4- (cycloheptanecarboxamido)cyclohexyl- amino)-3-(4-(morpholine-4- carbonyl)phenylamino)pyrazine-2- carboxamide |
| B-31 | 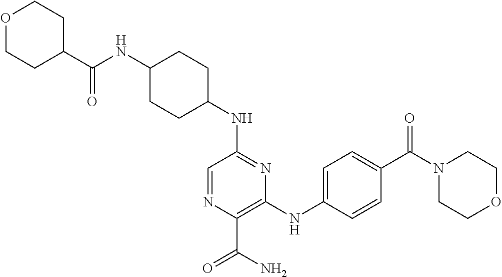 [SMILES: NC(=O)c1ncc(NC2CCC(NC(=O)C3CCOCC3)CC2)nc1Nc1ccc(C(=O)N2CCOCC2)cc1] | 3-(4-(morpholine-4- carbonyl)phenylamino)-5-(4-(tetrahydro- 2H-pyran-4- carboxamido)cyclohexyl- amino)pyrazine-2-carboxamide |

### Example C-1: Synthesis of 3-[(3-methyl-1,2-thiazol-5-yl)amino]-5-{[(3R)-piperidin-3-yl]amino}pyrazine-2-carboxamide (1)

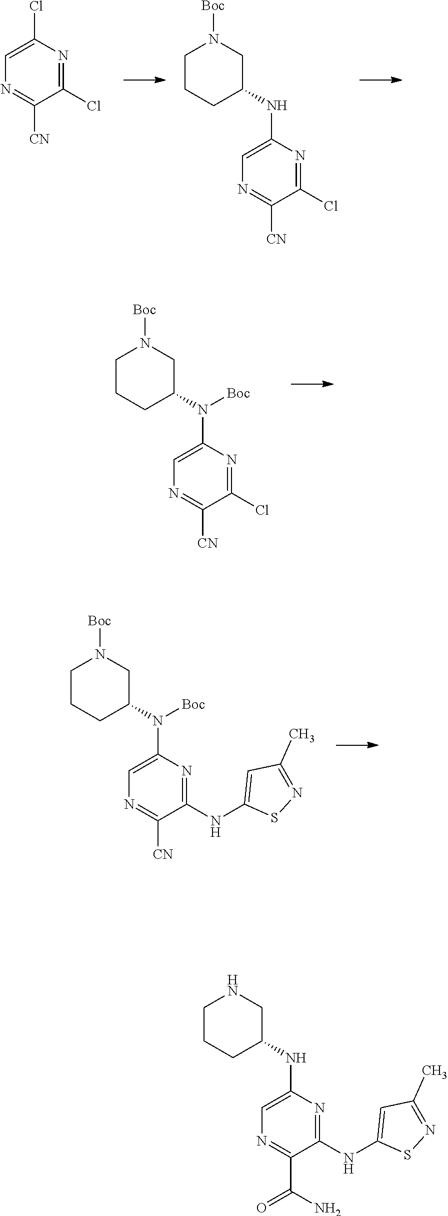 [SMILES: N#Cc1ncc(Cl)nc1Cl ; CC(C)(C)OC(=O)N1CCC[C@@H](Nc2cnc(C#N)c(Cl)n2)C1 ; CC(C)(C)OC(=O)N1CCC[C@@H](N(C(=O)OC(C)(C)C)c2cnc(C#N)c(Cl)n2)C1 ; Cc1cc(Nc2nc(N(C(=O)OC(C)(C)C)[C@@H]3CCCN(C(=O)OC(C)(C)C)C3)cnc2C#N)sn1 ; Cc1cc(Nc2nc(N[C@@H]3CCCNC3)cnc2C(N)=O)sn1]

To a solution of 3,5-dichloropyrazine-2-carbonitrile (1.00 g, 5.47 mmol) in DMF (10 mL) was added (R)-(−)-3-amino-1-Boc-piperidine (1.38 g, 6.9 mmol) and DIPEA (2.0 mL, 10.94 mmol) in a dropwise manner. The mixture was stirred at room temperature for 60 min. The reaction mixture was evaporated under reduced pressure and the residue was purified by flash chromatography with 0 to 50% ethyl acetate in cyclohexane to give tert-butyl (3R)-3-[(6-chloro-5-cyanopyrazin-2-yl)amino]piperidine-1-carboxylate (2.36 g, quantitative yield).

To a solution of tert-butyl (3R)-3-[(6-chloro-5-cyanopyrazin-2-yl)amino]piperidine-1-carboxylate (2.06 g, 6.1 mmol) in DCM (90 mL) was added a solution of Boc₂O (1.6 g, 7.32 mmol) in DCM (10 mL) and 4-DMAP (30 mg). The reaction mixture was refluxed for 2 h and, after cooling at room temperature, NaHCO₃ aqueous saturated solution (150 mL) was added to quench the reaction. The aqueous layer was extracted with DCM (3×100 mL), the organic layer was made dry with Na₂SO₄, filtered and concentrated under vacuum. The crude was purified by flash chromatography with 0 to 40% of ethyl acetate in cyclohexane to give tert-butyl (3R)-3-{[(tert-butoxy)carbonyl](6-chloro-5-cyanopyrazin-2-yl)amino}piperidine-1-carboxylate (1.86 g, 69% yield).

A mixture of tert-butyl (3R)-3-{[(tert-butoxy)carbonyl](6-chloro-5-cyanopyrazin-2-yl)amino}piperidine-1-carboxylate (0.63 g, 1.43 mmol), 3-methyl-1,2-thiazol-5-amine hydrochloride (0.65 g, 4.29 mmol), Pd(OAc)₂ (64 mg, 0.286 mmol), BINAP (178 mg, 0.286 mmol), fine powder Cs₂CO₃ (1.864 g, 5.72 mmol) in dioxane (20 mL) was degassed with a nitrogen stream for 10 min. The mixture was stirred in a nitrogen atmosphere at 115° C. overnight. The mixture was cooled, diluted with ethyl acetate, filtered through celite, and concentrated in vacuo. The residue was purified by flash chromatography with 0 to 50% ethyl acetate in cyclohexane to give tert-butyl (3R)-3-{[(tert-butoxy)carbonyl]({5-cyano-6-[(3-methyl-1,2-thiazol-5-yl)amino]pyrazin-2-yl})amino}piperidine-1-carboxylate (0.7 g, 94% yield).

Tert-butyl (3R)-3-{[(tert-butoxy)carbonyl]({5-cyano-6-[(3-methyl-1,2-thiazol-5-yl)amino]pyrazin-2-yl})amino}piperidine-1-carboxylate (50 mg, 0.097 mmol) was dissolved in TFA (2.5 mL) and then concentrated, sulfuric acid (0.5 mL) was added. The mixture was stirred at 60° C. for 30 min the solvent was removed under vacuum. The obtained solid was dissolved in a mixture of MeOH/H₂O and it was passed through SCX cartridge. The SCX cartridge was eluted with NH₃ in MeOH 1M. and concentration of these fractions afforded 3-[(3-methyl-1,2-thiazol-5-yl)amino]-5-{[(3R)-piperidin-3-yl]amino}pyrazine-2-carboxamide (25 mg, 77.3% yield). MS found for C14H19N7OS as (M+H)⁺ 334.0. ¹H NMR (400 MHz, DMSO) δ 12.21 (s, 1H), 7.91 (br. s., 1H), 7.78 (br. s., 1H), 7.48 (s, 1H), 7.42 (br. s., 1H), 6.82 (s, 1H), 4.08 (br. s., 1H), 3.16 (d, J=3.91 Hz, 2H), 2.94-2.82 (m, 1H), 2.63-2.52 (m, 2H), 2.28 (s, 3H), 2.13-2.00 (m, 1H), 1.70 (dd, J=8.22, 4.30 Hz, 1H), 1.55 (dt, J=13.40, 9.93 Hz, 1H), 1.48-1.34 (m, 1H).

### Example C-2: 5-{[(3S)-1-(dimethylcarbamoyl)piperidin-3-yl]amino}-3-[(3-methyl-1,2-thiazol-5-yl)amino]pyrazine-2-carboxamide (2)

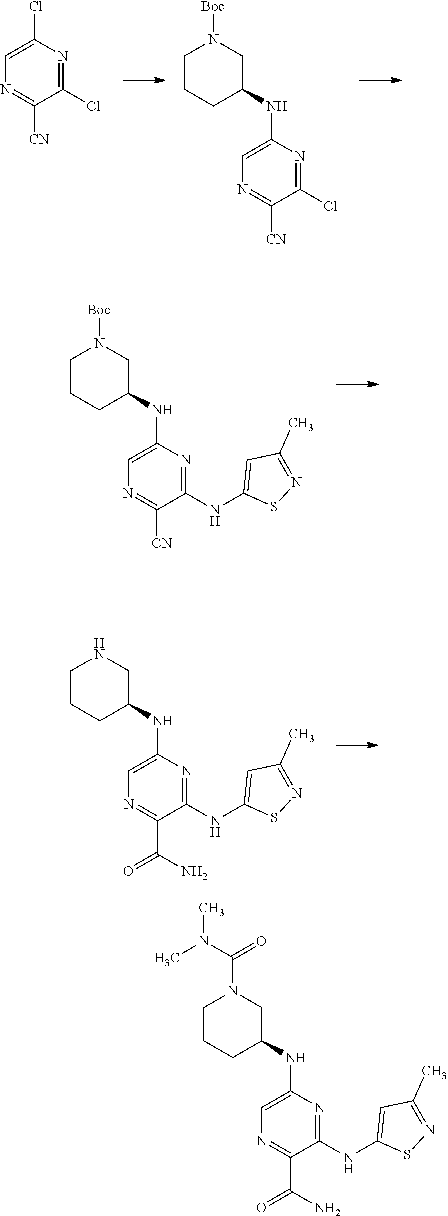

To a solution of 3,5-dichloropyrazine-2-carbonitrile (1.00 g, 5.47 mmol) in DMF (10 mL) was added (S)-(+)-3-Amino-1-Boc-piperidine (1.38 g, 6.9 mmol) and DIPEA (2.0 mL, 10.94 mmol) in a dropwise manner. The mixture was stirred at room temperature for 60 min. The reaction mixture was evaporated under reduced pressure and the residue was purified by flash chromatography with 0 to 50% ethyl acetate in cyclohexane to isolate tert-butyl (3S)-3-[(6-chloro-5-cyanopyrazin-2-yl)amino]piperidine-1-carboxylate (2.41 g, quantitative yield).

A mixture of tert-butyl (3S)-3-[(6-chloro-5-cyanopyrazin-2-yl)amino]piperidine-1-carboxylate (1 g, 3 mmol), 3-methyl-1,2-thiazol-5-amine hydrochloride (1.36 g, 9.0 mmol), Pd(OAc)₂ (340 mg, 1.5 mmol), BINAP (930 mg, 1.5 mmol), fine powder Cs₂CO₃ (3.9 g, 12 mmol) in dioxane (40 mL) was degassed with a nitrogen stream for 10 min. The mixture was stirred in a nitrogen atmosphere at 115° C. for 6 h, then cooled, diluted with ethyl acetate, filtered through celite, and concentrated in vacuo. The residue was purified by flash chromatography with 0 to 50% ethyl acetate in cyclohexane to give tert-butyl (3S)-3-({5-cyano-6-[(3-methyl-1,2-thiazol-5-yl)amino]pyrazin-2-yl}amino)piperidine-1-carboxylate (1.12 g, 90% yield).

Tert-butyl (3S)-3-({5-cyano-6-[(3-methyl-1,2-thiazol-5-yl)amino]pyrazin-2-yl}amino)piperidine-1-carboxylate (1.12 g, 2.7 mmol) was dissolved in TFA (10 mL) and then concentrated sulfuric acid (2 mL) was added. The mixture was stirred at 60° C. for 30 min, and then the solvent was removed under vacuum. The obtained solid was dissolved in a mixture of MeOH/H₂O and it was passed through SCX cartridge. 3-[(3-methyl-1,2-thiazol-5-yl)amino]-5-{[(3S)-piperidin-3-yl]amino}pyrazine-2-carboxamide was eluted with ammonia in methanol 1M (1.38 g, quant. yield).

To a mixture of 3-[(3-methyl-1,2-thiazol-5-yl)amino]-5-{[(3S)-piperidin-3-yl]amino}pyrazine-2-carboxamide (100 mg, 0.3 mmol), DIPEA (0.157 mL, 0.9 mmol) in DMF (3 mL) was added dimethylcarbamyl chloride (0.033 mL, 0.36 mmol) and the mixture was stirred 10 min at room temperature. The solvent was removed under vacuum and the crude was purified by flash chromatography with MeOH in DCM form 0 to 5% to give 5-{[(3S)-1-(dimethylcarbamoyl)piperidin-3-yl]amino}-3-[(3-methyl-1,2-thiazol-5-yl)amino]pyrazine-2-carboxamide (34 mg, 28% yield). MS found for C17H24N8O2S as (M+H)⁺ 405.0. ¹H NMR (400 MHz, DMSO) δ 12.23 (s, 1H), 8.00 (d, J=5.87 Hz, 1H), 7.82 (br. s., 1H), 7.49 (s, 1H), 7.45 (br. s., 1H), 6.82 (s, 1H), 4.18 (br. s., 1H), 3.52 (d, J=10.27 Hz, 1H), 3.36-3.32 (m, 1H), 2.95-2.89 (m, 2H), 2.70 (s, 6H), 2.27 (s, 3H), 2.13-2.01 (m, 1H), 1.85-1.76 (m, 1H), 1.67-1.46 (m, 2H).

### Example C-3: Synthesis of 5-{[(3R)-1-benzoylpiperidin-3-yl]amino}-3-[(3-methyl-1,2-thiazol-5-yl)amino]pyrazine-2-carboxamide (3)

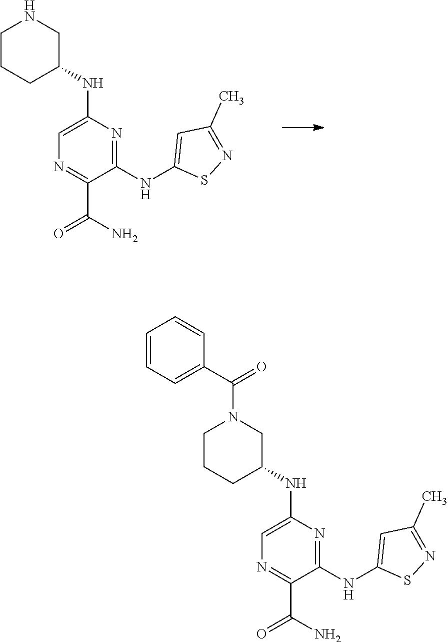 [SMILES: Cc1cc(Nc2nc(N[C@@H]3CCCNC3)cnc2C(N)=O)sn1 ; Cc1cc(Nc2nc(N[C@@H]3CCCN(C(=O)c4ccccc4)C3)cnc2C(N)=O)sn1]

In a similar manner as described in Example C-1, 3-[(3-methyl-1,2-thiazol-5-yl)amino]-5-[(3R)-piperidin-3-yl]amino) pyrazine-2-carboxamide was prepared. 3-[(3-methyl-1,2-thiazol-5-yl)amino]-5-{[(3R)-piperidin-3-yl]amino}pyrazine-2-carboxamide (60 mg, 0.18 mmol) was dissolved in DMF (2 mL) and DIPEA (0.042 mL), then benzoyl chloride (0.042 mL, 0.36 mmol) was added and the mixture was stirred for 1 h at room temperature. The solvent was removed and the crude was purified by flash chromatography with MeOH in DCM from 0 to 7% to obtain 5-{[(3R)-1-benzoylpiperidin-3-yl]amino}-3-[(3-methyl-1,2-thiazol-5-yl)amino]pyrazine-2-carboxamide (32 mg, 40.6% yield). MS found for C21H23N7O2S as (M+H)⁺ 438.4. ¹H NMR (400 MHz, DMSO) δ 12.47-11.82 (m, 1H), 8.31-6.56 (m, 9H), 4.47-3.08 (m, 6H), 2.33-1.44 (m, 7H).

### Example C-4: Synthesis of 5-{[(3S)-1-[4-(dimethylamino)benzoyl]piperidin-3-yl]amino}-3-[(3-methyl-1,2-thiazol-5-yl)amino]pyrazine-2-carboxamide (4)

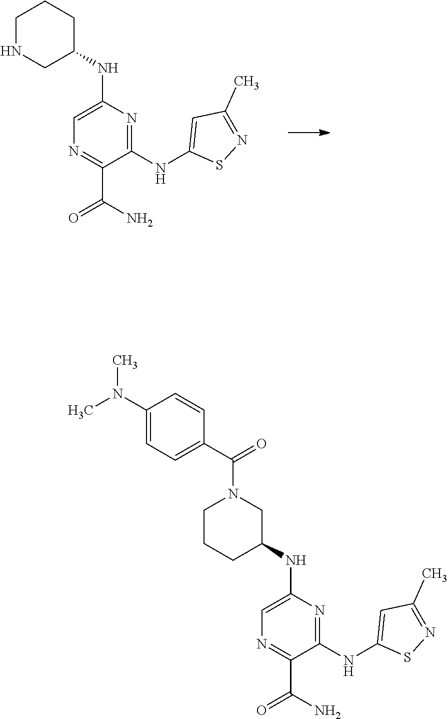 [SMILES: Cc1cc(Nc2nc(N[C@H]3CCCNC3)cnc2C(N)=O)sn1 ; Cc1cc(Nc2nc(N[C@H]3CCCN(C(=O)c4ccc(N(C)C)cc4)C3)cnc2C(N)=O)sn1]

In a similar manner as described in Example C-1, 5-{[(3S)-1-[4-(dimethylamino)benzoyl]piperidin-3-yl]amino}-3-[(3-methyl-1,2-thiazol-5-yl)amino]pyrazine-2-carboxamide (4) was prepared using 4-(dimethylamino) benzoyl chloride. MS found for C17H24N8O2S as (M+H)⁺ 481.0. ¹H NMR (400 MHz, DMSO) δ 12.15 (s, 1H), 7.91 (br. s., 1H), 7.83 (br. s., 1H), 7.51 (s, 1H), 7.44 (br. s., 1H), 7.06 (br. s., 2H), 6.78 (s, 1H), 6.38 (br. s., 2H), 3.32 (s, 5H), 2.83 (br. s., 6H), 2.28 (s, 3H), 2.16-2.03 (m, 1H), 2.00-1.85 (m, 1H), 1.77-1.65 (m, 1H), 1.63-1.51 (m, 1H).

### Example C-5: Synthesis of 5-{[(3R)-1-(dimethylcarbamoyl)piperidin-3-yl]amino}-3-[(3-methyl-1,2-thiazol-5-yl)amino]pyrazine-2-carboxamide hydrochloride (5)

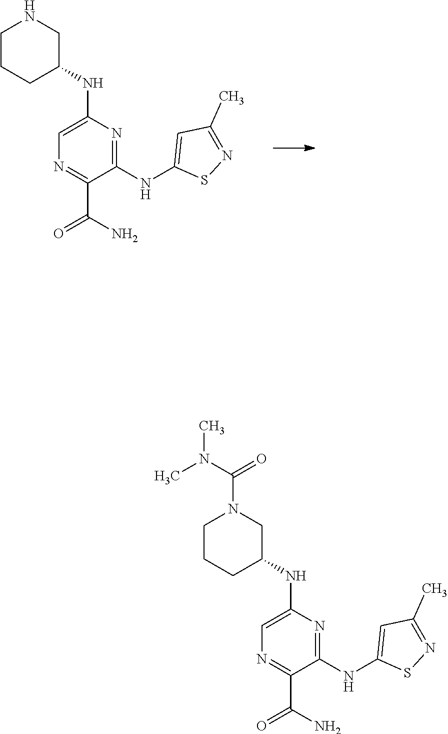 [SMILES: Cc1cc(Nc2nc(N[C@@H]3CCCNC3)cnc2C(N)=O)sn1 ; Cc1cc(Nc2nc(N[C@@H]3CCCN(C(=O)N(C)C)C3)cnc2C(N)=O)sn1]

In a similar manner as described in Example C-3, 5-{[(3R)-1-(dimethylcarbamoyl)piperidin-3-yl]amino}-3-[(3-methyl-1,2-thiazol-5-yl)amino]pyrazine-2-carboxamide hydrochloride (5) was prepared using dimethylcarbamyl chloride. MS found for C17H24N8O2S as (M+H)⁺ 405.0. ¹H NMR (400 MHz, DMSO) δ 12.27 (s, 1H), 8.05 (d, J=5.87 Hz, 1H), 7.83 (br. s., 1H), 7.63-7.35 (m, 2H), 6.86 (s, 1H), 4.65-3.96 (m, 1H), 3.53 (d, J=10.76 Hz, 1H), 3.39-3.27 (m, 1H), 2.99-2.85 (m, 2H), 2.71 (s, 6H), 2.29 (s, 3H), 2.14-1.99 (m, 1H), 1.87-1.75 (m, 1H), 1.69-1.44 (m, 2H).

### Example C-6: Synthesis of 5-{[(3S)-1-benzoylpiperidin-3-yl]amino}-3-[(3-methyl-1,2-thiazol-5-yl)amino]pyrazine-2-carboxamide (6)

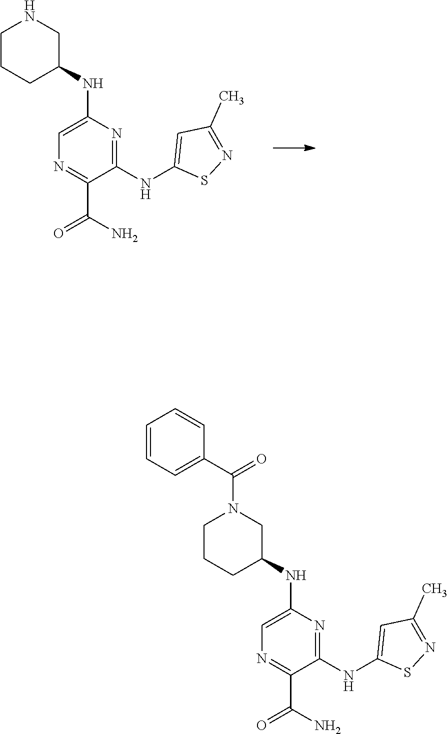 [SMILES: Cc1cc(Nc2nc(N[C@H]3CCCNC3)cnc2C(N)=O)sn1 ; Cc1cc(Nc2nc(N[C@H]3CCCN(C(=O)c4ccccc4)C3)cnc2C(N)=O)sn1]

In a similar manner as described in Example C-2, 5-{[(3S)-1-benzoylpiperidin-3-yl]amino}-3-[(3-methyl-1,2-thiazol-5-yl)amino]pyrazine-2-carboxamide (6) was prepared using benzoyl chloride. MS found for C21H23N7O2S as (M+H)⁺ 438.0. ¹H NMR (400 MHz, DMSO) δ 11.82-12.47 (m, 1H), 6.56-8.31 (m, 9H), 3.08-4.47 (m, 6H), 1.44-2.33 (m, 7H).

### Example C-7: Synthesis of 5-{[(3R)-1-[4-(dimethylamino)benzoyl]piperidin-3-yl]amino}-3-[(3-methyl-1,2-thiazol-5-yl)amino]pyrazine-2-carboxamide hydrochloride (7)

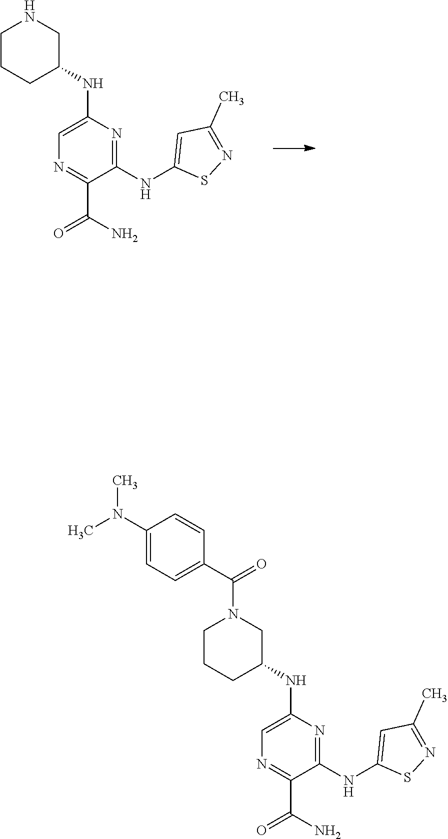 [SMILES: Cc1cc(Nc2nc(N[C@@H]3CCCNC3)cnc2C(N)=O)sn1 ; Cc1cc(Nc2nc(N[C@@H]3CCCN(C(=O)c4ccc(N(C)C)cc4)C3)cnc2C(N)=O)sn1]

In a similar manner as described in Example C-3, 5-{[(3R)-1-[4-(dimethylamino)benzoyl]piperidin-3-yl]amino}-3-[(3-methyl-1,2-thiazol-5-yl)amino]pyrazine-2-carboxamide hydrochloride (7) was prepared using 4-(dimethylamino)benzoyl chloride. MS found for C23H28N8O2S as (M+H)⁺ 481.0. ¹H NMR (400 MHz, DMSO) δ 12.24 (br.s., 1H), 8.13-7.94 (m, 1H), 7.80 (br. s, 1H), 7.55 (s, 1H), 7.48 (br. s, 1H), 7.23-7.01 (m, 2H), 6.85 (s, 2H), 6.74-6.35 (m, 2H), 4.19-3.31 (m, 5H), 2.86 (br. s., 6H), 2.31 (s, 3H), 2.16-2.03 (m, 1H), 1.98-1.86 (m, 1H), 1.80-1.66 (m, 1H), 1.64-1.52 (m, 1H).

### Example C-8: Synthesis of 5-{[(3S)-1-(dimethylcarbamoyl)piperidin-3-yl](methyl)amino}-3-[(3-methyl-1,2-thiazol-5-yl)amino]pyrazine-2-carboxamide (8)

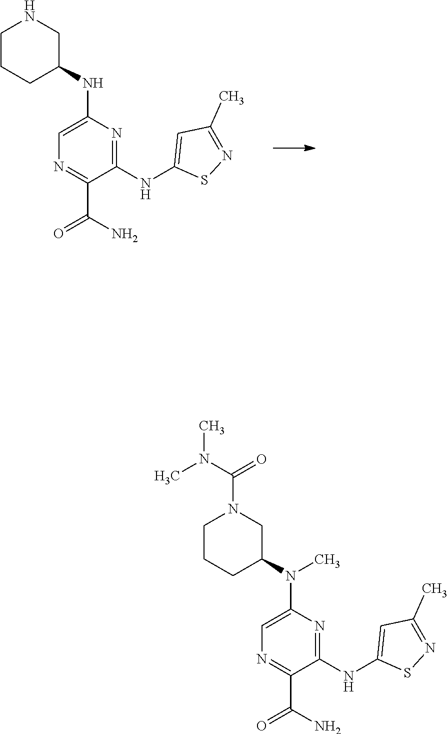 [SMILES: Cc1cc(Nc2nc(N[C@H]3CCCNC3)cnc2C(N)=O)sn1 ; Cc1cc(Nc2nc(N(C)[C@H]3CCCN(C(=O)N(C)C)C3)cnc2C(N)=O)sn1]

In a similar manner as described in Example C-2, 5-{[(3S)-1-(dimethylcarbamoyl)piperidin-3-yl](methyl)amino}-3-[(3-methyl-1,2-thiazol-5-yl)amino]pyrazine-2-carboxamide (8) was prepared using dimethylcarbamyl chloride. MS found for C18H26N8O2S as (M+H)⁺ 417.1. ¹H NMR (400 MHz, DMSO) δ 12.29 (s, 1H), 7.98-7.63 (m, 2H), 7.54 (br. s., 1H), 6.84 (s, 1H), 4.70 (br. s., 1H), 3.63-3.45 (m, 2H), 3.13 (br. s., 3H), 2.91 (t, J=12.20 Hz, 1H), 2.80-2.67 (m, 7H), 2.29 (s, 3H), 1.94-1.55 (m, 4H).

### Example C-9: Synthesis of 5-{[(3S)-1-[4-(dimethylamino)benzoyl]piperidin-3-yl](methyl)amino}-3-[(3-methyl-1,2-thiazol-5-yl)amino]pyrazine-2-carboxamide (9)

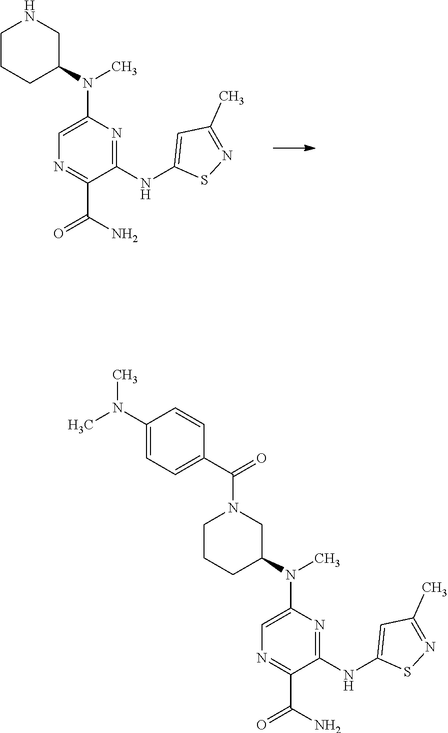 [SMILES: Cc1cc(Nc2nc(N(C)[C@H]3CCCNC3)cnc2C(N)=O)sn1 ; Cc1cc(Nc2nc(N(C)[C@H]3CCCN(C(=O)c4ccc(N(C)C)cc4)C3)cnc2C(N)=O)sn1]

In a similar manner as described in Example C-2, 5-{[(3S)-1-[4-(dimethylamino)benzoyl]piperidin-3-yl](methyl)amino}-3-[(3-methyl-1,2-thiazol-5-yl)amino]pyrazine-2-carboxamide (9) was prepared using 4-(dimethylamino)benzoyl chloride. MS found for C24H30N8O2S as (M+H)⁺ 495.0. ¹H NMR (400 MHz, DMSO) δ 12.29 (s, 1H), 7.91 (br. s., 1H), 7.70 (br. s., 1H), 7.56 (br. s., 1H), 7.28 (d, J=8.23 Hz, 2H), 6.86 (s, 1H), 6.75-6.55 (m, 2H), 5.03-2.34 (m, 14H), 2.30 (s, 3H), 2.05-1.58 (m, 4H).

### Example C-10: Synthesis of 5-{[(3S)-1-(dimethylcarbamoyl)pyrrolidin-3-yl]amino}-3-[(3-methyl-1,2-thiazol-5-yl)amino]pyrazine-2-carboxamide (10)

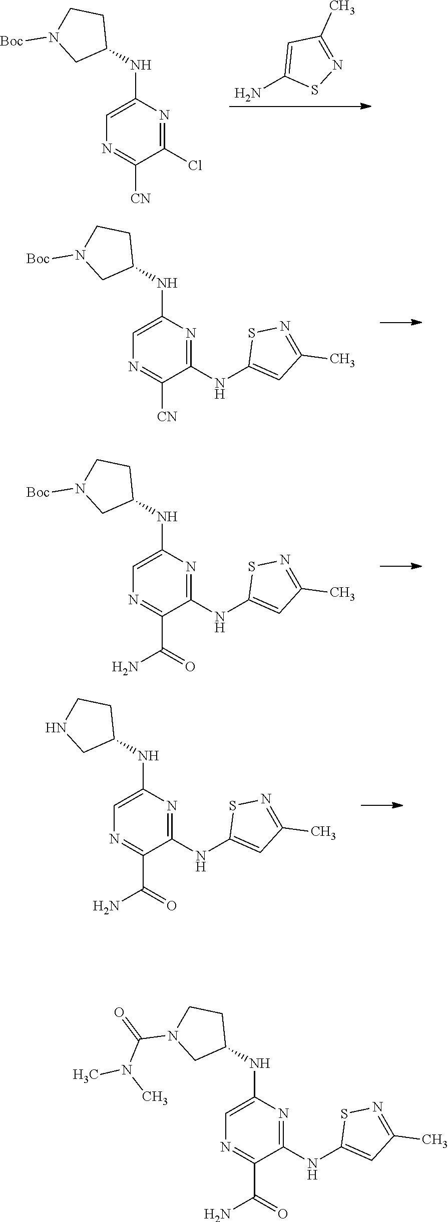

A mixture of Tert-butyl (3R)-3-[(6-chloro-5-cyanopyrazin-2-yl)amino]pyrrolidine-1-carboxylate (0.701 g, 2.16 mmol), 5-amino-3-methyl-isothiazole hydrochloride (975 mg, 6.48 mmol), Pd(OAc)₂ (100 mg, 0.445 mmol), BINAP (270 mg, 0.43 mmol), fine powder Cs₂CO₃ (2.82 g, 8.64 mmol) in dioxane (30 mL) was degassed with a nitrogen stream for 10 min. The mixture was stirred in a nitrogen atmosphere at 115° C. overnight, then cooled, diluted with ethyl acetate, filtered through celite, and concentrated in vacuo. The residue was purified by flash chromatography with 0 to 70% ethyl acetate in cyclohexane to give (3S)-3-({5-cyano-6-[(3-methyl-1,2-thiazol-5-yl)amino]pyrazin-2-yl}amino)pyrrolidine-1-carboxylate (835 mg, 96% yield) as yellow solid. MS found for C18H23N7O2S as (M+H)⁺ 402.0.

(3S)-3-({5-cyano-6-[(3-methyl-1,2-thiazol-5-yl)amino]pyrazin-2-yl}amino)pyrrolidine-1-carboxylate (835 mg, 2.08 mmol) was dissolved in a mixture of 5 mL DMSO and 5 mL MeOH and stirred at room temperature. NaOH (200 mg) was added followed by 1 mL 30% H₂O₂. The mixture was stirred at room temperature for 1 h then at 50° C. for further 12 hours. 1 mL of 30% H₂O₂ was added and the mixture was stirred at room temperature for further 20 minutes. The mixture was diluted with 10 mL of acetonitrile and stirred for 10 minutes. Ethyl acetate (100 ml) was added, the solid was filtrated off and the organic solution was concentrated in vacuo to afford crude tert-butyl (3 S)-3-({5-carbamoyl-6-[(3-methyl-1,2-thiazol-5-yl)amino]pyrazin-2-yl}amino)pyrrolidine-1-carboxylate as a red/orange oil that was treated with 50 mL of HCl 4N in dioxane at room temperature for 2 hours. The mixture was concentrated in vacuo and evaporated to afford a crude product that was purified by SCX cartridge followed by flash chromatography (silica) eluent MeOH in DCM from 0 to 10% to give 3-[(3-methyl-1,2-thiazol-5-yl)amino]-5-{[(3S)-pyrrolidin-3-yl]amino}pyrazine-2-carboxamide (100.0 mg, 14% yield). MS found for C13H17N7OS as (M+H)⁺ 319.9.

In a similar manner as described in Example C-2, 5-{[(3S)-1-(dimethylcarbamoyl)pyrrolidin-3-yl]amino}-3-[(3-methyl-1,2-thiazol-5-yl)amino]pyrazine-2-carboxamide (10) was prepared using dimethylcarbamyl chloride. MS found for C16H22N8O2S as (M+H)⁺ 391.0. ¹H NMR (400 MHz, DMSO) δ 12.29 (s, 1H), 8.32-8.17 (m, 1H), 7.92-7.77 (m, 1H), 7.59-7.42 (m, 2H), 6.87 (s, 1H), 4.71-4.48 (m, 1H), 3.79-3.73 (m, 1H), 3.48-3.57 (m, 1H), 3.44-3.39 (m, 1H), 3.31-3.23 (m, 1H), 2.75 (s, 6H), 2.30 (s, 3H), 2.28-2.17 (m, 1H), 1.97-1.86 (m, 1H).

### Example C-11: Synthesis of 5-{[(3R)-1-benzoylpyrrolidin-3-yl]amino}-3-[(3-methyl-1,2-thiazol-5-yl)amino]pyrazine-2-carboxamide hydrochloride (11)

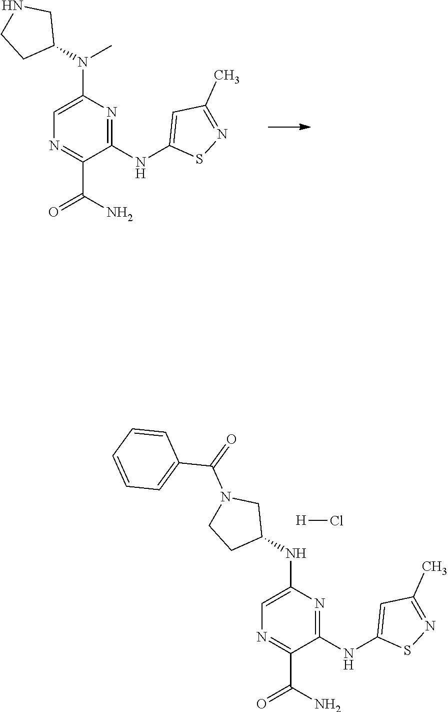

In a similar manner as described in Example C-2, 5-{[(3R)-1-benzoylpyrrolidin-3-yl]amino}-3-[(3-methyl-1,2-thiazol-5-yl)amino]pyrazine-2-carboxamide hydrochloride (11) was prepared using benzoyl chloride. MS found for C20H22ClN7O2S as (M+H)⁺ 424.3. ¹H NMR (400 MHz, DMSO) δ 12.50-12.06 (m, 1H), 8.68-8.21 (m, 1H), 8.03-7.72 (m, 1H), 7.69-7.30 (m, 7H), 7.07-6.58 (m, 1H), 4.84-4.59 (m, 1H), 4.00-3.29 (m, 4H), 2.31 (d, J=9.78 Hz, 5H).

### Example C-12: Synthesis of 5-{[(3R)-1-(dimethylcarbamoyl)pyrrolidin-3-yl]amino}-3-[(3-methyl-1,2-thiazol-5-yl)amino]pyrazine-2-carboxamide (12)

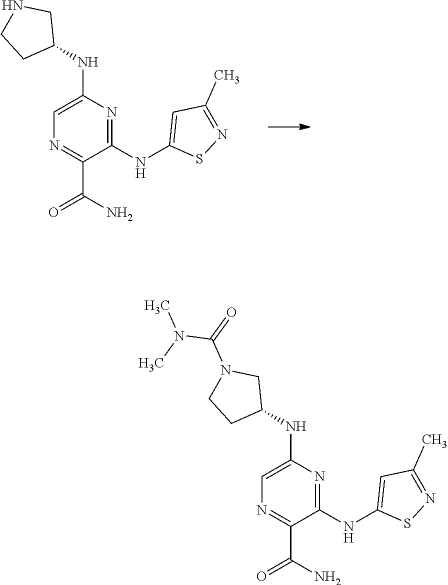 [SMILES: Cc1cc(Nc2nc(N[C@@H]3CCNC3)cnc2C(N)=O)sn1 ; Cc1cc(Nc2nc(N[C@@H]3CCN(C(=O)N(C)C)C3)cnc2C(N)=O)sn1]

In a similar manner as described in Example C-2, 5-{[(3R)-1-(dimethylcarbamoyl)pyrrolidin-3-yl]amino}-3-[(3-methyl-1,2-thiazol-5-yl)amino]pyrazine-2-carboxamide (12) was prepared using dimethylcarbamyl chloride. MS found for C16H22N8O2S as (M+H)⁺ 391.0. ¹H NMR (400 MHz, DMSO) δ 12.27 (s, 1H), 8.23 (d, J=5.38 Hz, 1H), 7.84 (br. s., 1H), 7.49 (s, 1H), 7.47 (br. s., 1H), 6.85 (s, 1H), 4.66-4.55 (m, 1H), 3.76 (dd, J=10.76, 5.87 Hz, 1H), 3.57-3.49 (m, 1H), 3.45-3.36 (m, 1H), 3.28 (dd, J=11.00, 3.67 Hz, 1H), 2.75 (s, 6H), 2.29 (s, 3H), 2.27-2.19 (m, 1H), 1.96-1.85 (m, 1H).

### Example C-13: Synthesis of 5-{[(3R)-1-[4-(dimethylamino)benzoyl]pyrrolidin-3-yl]amino}-3-[(3-methyl-1,2-thiazol-5-yl)amino]pyrazine-2-carboxamide (13)

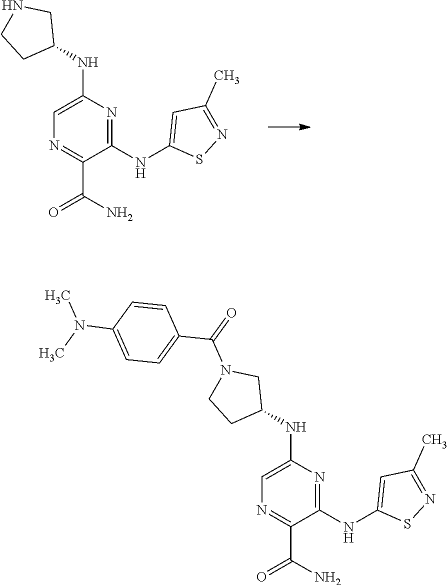 [SMILES: Cc1cc(Nc2nc(N[C@@H]3CCNC3)cnc2C(N)=O)sn1 ; Cc1cc(Nc2nc(N[C@@H]3CCN(C(=O)c4ccc(N(C)C)cc4)C3)cnc2C(N)=O)sn1]

In a similar manner as described in Example C-2, 5-{[(3R)-1-[4-(dimethylamino)benzoyl]pyrrolidin-3-yl]amino}-3-[(3-methyl-1,2-thiazol-5-yl)amino]pyrazine-2-carboxamide (13) was prepared using 4-(dimethylamino)benzoyl chloride. MS found for C22H26N8O2S as (M+H)⁺ 465.1. ¹H NMR (400 MHz, DMSO) δ 12.25 (br. s., 1H), 8.48-8.15 (m, 1H), 7.84 (br. s., 1H), 7.47 (br. s., 4H), 6.84 (br. s., 1H), 6.67 (br. s., 2H), 4.69 (br. s., 1H), 4.13-3.83 (m, 1H), 3.78-3.69 (m, 1H), 3.67-3.46 (m, 2H), 2.93 (br. s., 6H), 2.29 (s, 3H), 2.36-2.26 (m, 1H), 2.07-1.96 (m, 1H).

### Example C-14: Synthesis of 5-({[(3R)-1-(dimethylcarbamoyl)piperidin-3-yl]amino}-3-[(3-methyl-1,2-thiazol-5-yl)amino]pyrazine-2-carboxamide hydrochloride (14)

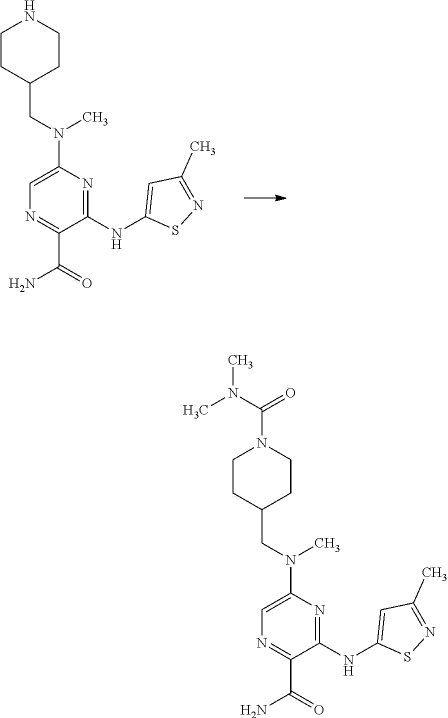 [SMILES: Cc1cc(Nc2nc(N(C)CC3CCNCC3)cnc2C(N)=O)sn1 ; Cc1cc(Nc2nc(N(C)CC3CCN(C(=O)N(C)C)CC3)cnc2C(N)=O)sn1]

In a similar manner as described in Example C-2, 5-({[1-(dimethylcarbamoyl)piperidin-4-yl]methyl}(methyl)amino)-3-[(3-methyl-1,2-thiazol-5-yl)amino]pyrazine-2-carboxamide (14) was prepared using dimethylcarbamyl chloride. MS found for C19H28N8O2S as (M+H)⁺ 433.4. ¹H NMR (400 MHz, DMSO) δ 12.31 (s, 1H), 7.91-7.78 (m, 1H), 7.72-7.60 (m, 1H), 7.57-7.45 (m, 1H), 6.84 (s, 1H), 3.80-3.62 (m, 2H), 3.55 (d, J=12.91 Hz, 2H), 3.26 (br. s., 3H), 2.69 (s, 6H), 2.61 (t, J=11.93 Hz, 2H), 2.29 (s, 3H), 2.09-1.91 (m, 1H), 1.62 (d, J=11.35 Hz, 2H), 1.26 (qd, J=12.06, 2.93 Hz, 2H).

### Example C-15: 5-{[(1-benzoylpiperidin-4-yl)methyl](methyl)amino}-3-[(3-methyl-1,2-thiazol-5-yl)amino]pyrazine-2-carboxamide (15)

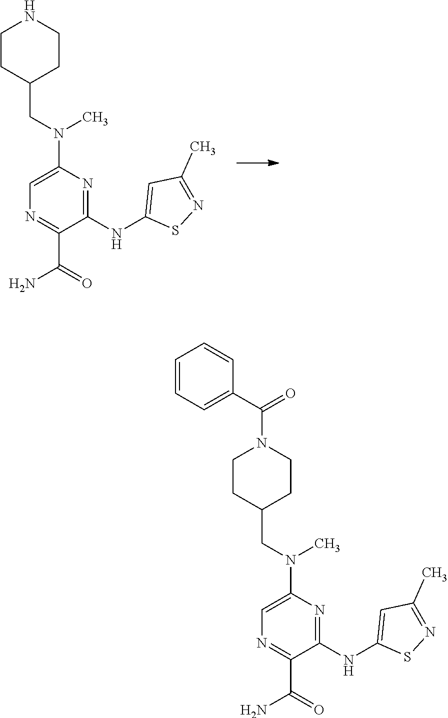 [SMILES: Cc1cc(Nc2nc(N(C)CC3CCNCC3)cnc2C(N)=O)sn1 ; Cc1cc(Nc2nc(N(C)CC3CCN(C(=O)c4ccccc4)CC3)cnc2C(N)=O)sn1]

In a similar manner as described in Example C-2, 5-{[(1-benzoylpiperidin-4-yl)methyl](methyl)amino}-3-[(3-methyl-1,2-thiazol-5-yl)amino]pyrazine-2-carboxamide (15) was prepared using benzoyl chloride. MS found for C23H27N7O2S as (M+H)⁺ 466.4. ¹H NMR (400 MHz, DMSO) δ 12.32 (s, 1H), 7.84 (br. s., 1H), 7.66 (br. s., 1H), 7.52 (br. s., 1H), 7.46-7.39 (m, 3H), 7.37-7.27 (m, 2H), 6.85 (s, 1H), 4.49 (br. s., 1H), 3.92-3.43 (m, 3H), 3.26 (br. s., 3H), 3.09-2.87 (m, 1H), 2.84-2.60 (m, 1H), 2.30 (s, 3H), 2.14 (s, 1H), 1.90-1.46 (m, 2H), 1.31 (s, 1H), 1.41-1.15 (m, 2H).

### Example C-16: 5-[({1-[4-(dimethylamino)benzoyl]piperidin-4-yl}methyl)(methyl)amino]-3-[(3-methyl-1,2-thiazol-5-yl)amino]pyrazine-2-carboxamide (16)

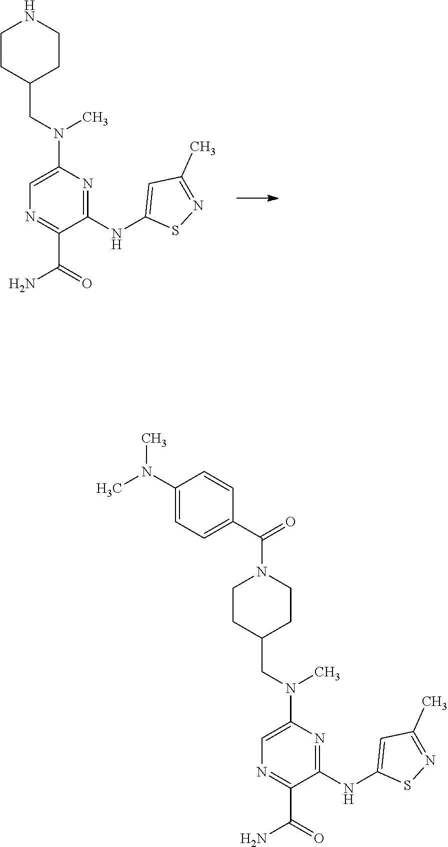 [SMILES: Cc1cc(Nc2nc(N(C)CC3CCNCC3)cnc2C(N)=O)sn1 ; Cc1cc(Nc2nc(N(C)CC3CCN(C(=O)c4ccc(N(C)C)cc4)CC3)cnc2C(N)=O)sn1]

In a similar manner as described in Example C-2, 5-[({(1-[4-(dimethylamino)benzoyl]piperidin-4-yl}methyl)(methyl)amino]-3-[(3-methyl-1,2-thiazol-5-yl)amino]pyrazine-2-carboxamide (16) was prepared using 4-(dimethylamino)benzoyl chloride. MS found for C25H32N8O2S as (M+H)⁺ 509.4. ¹H NMR (400 MHz, DMSO) δ 12.26 (s, 1H), 7.84 (br. s, 1H), 7.68 (br. s, 1H), 7.52 (br. s, 1H), 7.22 (d, J=8.61 Hz, 2H), 6.84 (s, 1H), 6.69 (d, J=8.61 Hz, 2H), 4.39-3.89 (m, 2H), 3.83-3.54 (m, 2H), 3.26 (br. s., 3H), 2.93 (s, 6H), 2.89-2.73 (m, 2H), 2.30 (s, 3H), 2.22-1.99 (m, 1H), 1.67 (d, J=11.35 Hz, 2H), 1.27 (qd, J=12.26, 3.52 Hz, 2H).

### Example C-17: Synthesis of (S)-3-(1-acryloylpiperidin-3-ylamino)-5-(4-isopropylphenylamino)-1,2,4-triazine-6-carboxamide (17)

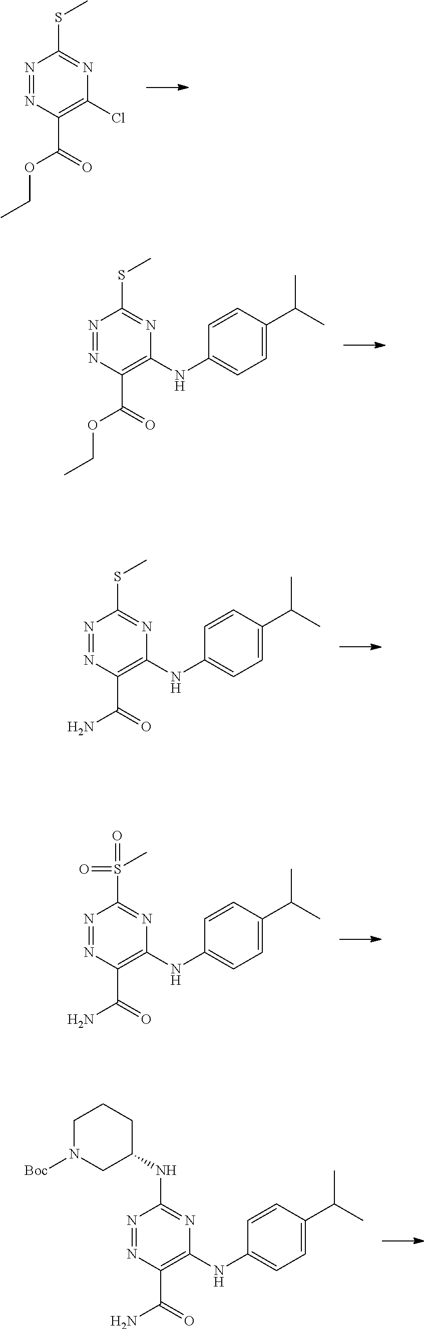 [SMILES: CCOC(=O)c1nnc(SC)nc1Cl ; CCOC(=O)c1nnc(SC)nc1Nc1ccc(C(C)C)cc1 ; CSc1nnc(C(N)=O)c(Nc2ccc(C(C)C)cc2)n1 ; CC(C)c1ccc(Nc2nc(S(C)(=O)=O)nnc2C(N)=O)cc1 ; CC(C)c1ccc(Nc2nc(N[C@H]3CCCN(C(=O)OC(C)(C)C)C3)nnc2C(N)=O)cc1]

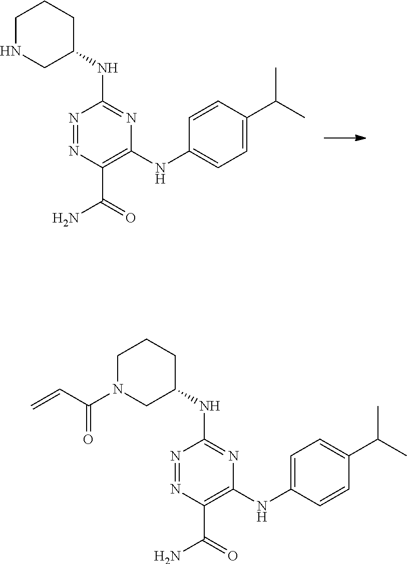

To a light yellow solution of commercially available ethyl 5-chloro-3-(methylthio)-1,2,4-triazine-6-carboxylate (3.00 g, 12.88 mmol) was added 4-isopropylaniline (2.2 ml, 15.45 mmol), resulting in gelatinous yellow slurry. The mixture was treated dropwise with DIPEA (2.7 ml, 15.45 mmol), resulting in a clear brown solution. After 5 minutes of stirring, LCMS confirmed clean transformation into ethyl 5-(4-isopropylphenylamino)-3-(methylthio)-1,2,4-triazine-6-carboxylate; MW=332.4, MH⁺=333.3.

To the reaction mixture was added 7N ammonia in methanol (74 ml, 515 mmol). Within 30 minutes of stirring, pale yellow solids started to form out of the solution. The solution was stirred for a total of 4 hours, upon which time the pale yellow solids were isolated by a disposable ChemGlass filter funnel (cat# OP-6602-12), washed with cold acetonitrile (2×20 ml) and cold hexanes (2×25 ml), then air-vacuum dried for 1 hr to isolate 5-(4-isopropylphenylamino)-3-(methylthio)-1,2,4-triazine-6-carboxamide (3.35 g, 86% yield); MW=303.4, MH⁺=304.0.

To a yellow solution of 5-(4-isopropylphenylamino)-3-(methylthio)-1,2,4-triazine-6-carboxamide (3.35 g, 11.04 mmol) in 245 ml THF, under the atmosphere of nitrogen, was added dry mCPBA (7.42 g, 33.13 mmol) in small portions. The resulting solution became yellow slurry within 1 hr. At the 2 hr time point, LCMS showed progressing oxidation with a 1:5 ratio of sulfoxide/sulfone products. The mixture was stirred for an additional 2 hrs, to allow maximum product precipitation, then was filtered through a disposable ChemGlass filter funnel (cat# OP-6602-12), washed with cold DCM (4×15 ml), and air-vacuum dried for overnight to produce 5-(4-isopropylphenylamino)-3-(methylsulfonyl)-1,2,4-triazine-6-carboxamide (2.91 g, 78%) as yellow powder; MW=335.4, MH⁺=336.0.

A yellow solution of 5-(4-isopropylphenylamino)-3-(methylsulfonyl)-1,2,4-triazine-6-carboxamide (750 mg, 2.24 mmol), (S)-tert-butyl 3-aminopiperidine-1-carboxylate (896 mg, 4.47 mmol) and DIPEA (0.78 ml, 4.47 mmol) in 30 ml NMP was heated under nitrogen for 2 hours, then cooled to room temperature. 50 ml saturated aqueous NaHCO₃ was added to the solution, resulting in white precipitate which was isolated by filtration and confirmed by LCMS to be the desired product, (MW=455.6, MH⁺=456.5). The solid was washed with water and air-vacuum dried overnight. The isolated 2.2 g of this material was purified further by flash chromatography using 0 to 100% EtOAc in hexanes to give (S)-tert-butyl 3-(6-carbamoyl-5-(4-isopropylphenylamino)-1,2,4-triazin-3-ylamino)piperidine-1-carboxylate (660 mg, 65%) as a yellow crystalline solid.

(S)-tert-butyl 3-(6-carbamoyl-5-(4-isopropylphenylamino)-1,2,4-triazin-3-ylamino)piperidine-1-carboxylate (660 mg, 1.45 mmol) was treated with 10 ml of 4N HCl in dioxane at RT for 1.5 hrs, then concentrated in vacuo to afford crude (S)-5-(4-isopropylphenylamino)-3-(piperidin-3-ylamino)-1,2,4-triazine-6-carboxamide, (700 mg, assume quantitative) as a bright yellow crystalline solid, HCl-salt; MW=355.4, MH⁺=356.2.

To a solution of (S)-5-(4-isopropylphenylamino)-3-(piperidin-3-ylamino)-1,2,4-triazine-6-carboxamide (20 mg, 0.05 mmol) in 1.5 ml NMP was added DIPEA (62 μl, 0.36 mmol), and 2 minutes later acryloyl chloride (7.0 μl, 0.087 mmol). Allowed to stir for 10 minutes, then quenched with 0.2 ml TFA. The mixture was diluted with 6 ml water and directly subjected to reverse phase preparative HPLC using 0.1% formic acid in water and CH₃CN as mobile phases. The product, (S)-3-(1-acryloylpiperidin-3-ylamino)-5-(4-isopropylphenylamino)-1,2,4-triazine-6-carboxamide was isolated as a pale yellow solid, formic acid salt (8.2 mg, 39%); MW=409.5, MH⁺=410.3, MH⁺=408.2.

### Example C-18: Synthesis of (S)-3-(1-(2-aminoacetyl)piperidin-3-ylamino)-5-(4-isopropylphenylamino)-1,2,4-triazine-6-carboxamide (18)

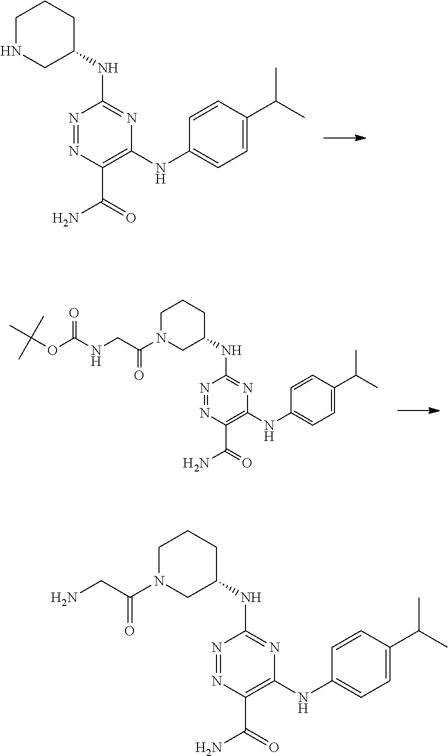

2-(tert-butoxycarbonylamino)acetic acid (67 mg, 0.38 mmol), EDC (147 mg, 0.77 mmol), HOBt.H₂O (117 mg, 0.77 mmol) and TEA (178 μl, 1.28 mmol) were stirred in 1.3 ml DMF for 15 minutes. (S)-5-(4-isopropylphenylamino)-3-(piperidin-3-ylamino)-1,2,4-triazine-6-carboxamide (100 mg, 0.255 mmol) was added and stirring was continued for 1.5 hours. The resulting mixture was quenched with 3 ml water and diluted further with EtOAc (35 ml), water (7 ml) and NaHCO₃ sat. (7 ml). The phases were separated and the aqueous phase was re-extracted 1× with EtOAc. The combined organics were dried over Na₂SO₄, filtered and concentrated in vacuo to 0.4 g crude liquid. The crude material was purified by flash chromatography using 0 to 10% MeOH in DCM gradient to isolate (S)-tert-butyl 2-(3-(6-carbamoyl-5-(4-isopropylphenylamino)-1,2,4-triazin-3-ylamino)piperidin-1-yl)-2-oxoethylcarbamate (95.7 mg, 73%) as a clear yellow solid; MW=512.6, MH⁺=513.7.

(S)-tert-butyl 2-(3-(6-carbamoyl-5-(4-isopropylphenylamino)-1,2,4-triazin-3-ylamino)piperidin-1-yl)-2-oxoethylcarbamate (95.7 mg, 0.19 mmol) was dissolved in 2 ml 4N HCl in dioxane and allowed to sit for 2 hrs, then concentrated in vacuo. (S)-3-(1-(2-aminoacetyl)piperidin-3-ylamino)-5-(4-isopropylphenylamino)-1,2,4-triazine-6-carboxamide (95.2 mg, assume quantitative) was obtained as a pale yellow solid, HCl salt; MW=412.5, MH⁺=413.2, MH⁻=411.2.

### Example C-19: Synthesis of (S)-3-(1-(2-acrylamidoacetyl)piperidin-3-ylamino)-5-(4-isopropylphenylamino)-1,2,4-triazine-6-carboxamide (19)

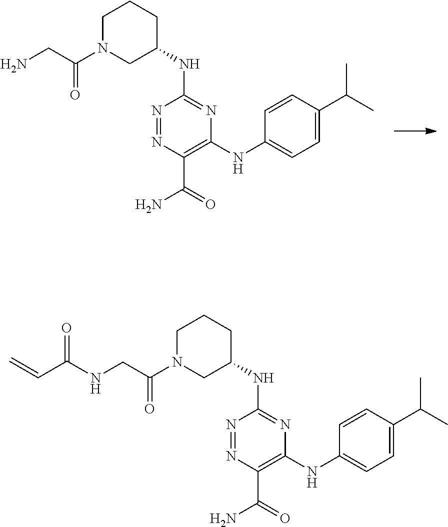

A solution of (S)-3-(1-(2-aminoacetyl)piperidin-3-ylamino)-5-(4-isopropylphenylamino)-1,2,4-triazine-6-carboxamide (20 mg, 0.044 mmol) in 1.5 ml NMP was treated with DIPEA (55 μl, 0.31 mmol), followed by acryloyl chloride (6.2 μl, 0.076 mmol). After 10 min of stirring the reaction was quenched by addition of 0.2 ml TFA. The mixture was diluted with 5 ml water and purified by reverse phase preparative HPLC, using 0.1% formic acid in water and CH₃CN as mobile phase, to give (S)-3-(1-(2-acrylamidoacetyl)piperidin-3-ylamino)-5-(4-isopropylphenylamino)-1,2,4-triazine-6-carboxamide (13.4 mg, 64%) as a light yellow solid, formic acid salt; MW=466.5, MH⁺=467.4, MH⁻=465.2.

### Example C-20: Synthesis of (S)-3-(1-(4-aminobutanoyl)piperidin-3-ylamino)-5-(4-isopropylphenylamino)-1,2,4-triazine-6-carboxamide (20)

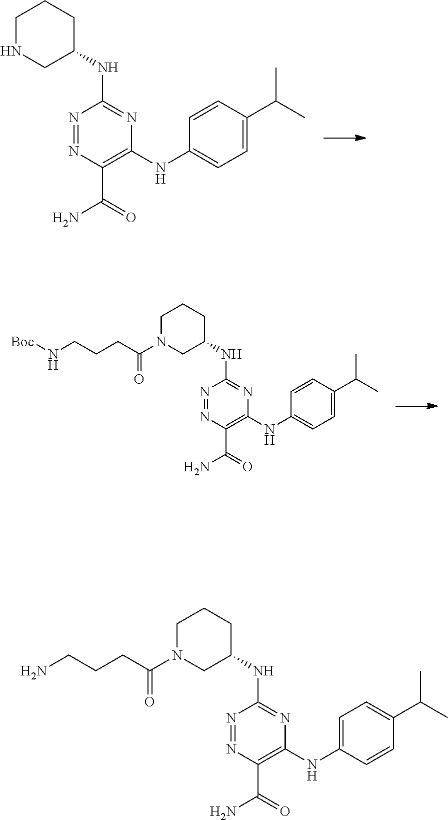 [SMILES: CC(C)c1ccc(Nc2nc(N[C@H]3CCCNC3)nnc2C(N)=O)cc1 ; CC(C)c1ccc(Nc2nc(N[C@H]3CCCN(C(=O)CCCNC(=O)OC(C)(C)C)C3)nnc2C(N)=O)cc1 ; CC(C)c1ccc(Nc2nc(N[C@H]3CCCN(C(=O)CCCN)C3)nnc2C(N)=O)cc1]

4-(tert-butoxycarbonylamino)butanoic acid (78 mg, 0.38 mmol), EDC (147 mg, 0.77 mmol), HOBt.H₂O (117 mg, 0.77 mmol) and TEA (178 μl, 1.28 mmol) were stirred in 1.3 ml DMF for 15 minutes. To the mixture was added (S)-5-(4-isopropylphenylamino)-3-(piperidin-3-ylamino)-1,2,4-triazine-6-carboxamide (100 mg, 0.255 mmol) and the stirring continued for 1.5 hours. The reaction was then quenched with 3 ml water and diluted further with EtOAc (35 ml), water (7 ml) and NaHCO₃ sat. (7 ml). The phases were separated and the aqueous re-extracted 1× with EtOAc. The combined organics were dried over Na₂SO₄, filtered and concentrated in vacuo to 0.6 g crude liquid. The crude was purified by flash chromatography using 0 to 10% MeOH in DCM gradient to isolate (S)-tert-butyl 4-(3-(6-carbamoyl-5-(4-isopropylphenylamino)-1,2,4-triazin-3-ylamino)piperidin-1-yl)-4-oxobutylcarbamate (104.6 mg, 78%) as a clear yellow solid; MW=540.7, MH⁺=541.8.

(S)-tert-butyl 4-(3-(6-carbamoyl-5-(4-isopropylphenylamino)-1,2,4-triazin-3-ylamino)piperidin-1-yl)-4-oxobutylcarbamate (104.6 mg, 0.19 mmol) was dissolved in 2 ml 4N HCl in dioxane and allowed to sit for 2 hrs, then concentrated in vacuo. Obtained (S)-3-(1-(4-aminobutanoyl)piperidin-3-ylamino)-5-(4-isopropylphenylamino)-1,2,4-triazine-6-carboxamide (100 mg, assume quantitative) as a pale yellow solid, HCl salt; MW=440.5, MH⁺=441.2, MH⁻=439.3.

### Example C-21: Synthesis of (S)-3-(1-(4-acrylamidobutanoyl)piperidin-3-ylamino)-5-(4-isopropylphenylamino)-1,2,4-triazine-6-carboxamide (21)

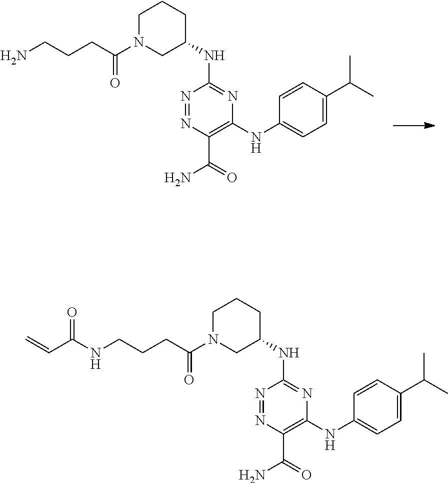

(S)-3-(1-(4-aminobutanoyl)piperidin-3-ylamino)-5-(4-isopropylphenylamino)-1,2,4-triazine-6-carboxamide (20 mg, 0.04 mmol) was dissolved in 1.5 ml dry NMP. To the solution were added DIPEA (51 ml, 0.29 mmol), and a few minutes later, acryloyl chloride (5.8 μl, 0.071 mmol). The mixture was stirred for 10 minutes, then quenched with 0.2 ml TFA. The mixture was diluted with 5 ml water and purified by reverse phase preparative HPLC, using 0.1% formic acid in water and CH₃CN as mobile phase, to give (S)-3-(1-(4-acrylamidobutanoyl)piperidin-3-ylamino)-5-(4-isopropylphenylamino)-1,2,4-triazine-6-carboxamide (9.4 mg, 45%), as a yellow solid, formic acid salt; MW=494.6, MH⁺=495.4, MH⁻=493.2.

### Example C-22: Synthesis of 3-((1-acryloylpiperidin-4-yl)methylamino)-5-(4-isopropylphenylamino)-1,2,4-triazine-6-carboxamide (22)

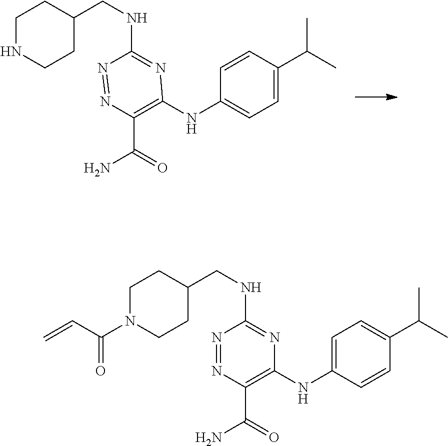

Compound 5-(4-isopropylphenylamino)-3-(piperidin-4-ylmethylamino)-1,2,4-triazine-6-carboxamide was prepared using the same synthetic procedure described for compound (S)-5-(4-isopropylphenylamino)-3-(piperidin-3-ylamino)-1,2,4-triazine-6-carboxamide in Example C-17, using 4-aminomethyl-1-Boc-piperidine.

To a solution of 5-(4-isopropylphenylamino)-3-(piperidin-4-ylmethylamino)-1,2,4-triazine-6-carboxamide (15 mg, 0.037 mmol) in 1.2 ml NMP were added DIPEA (45 μl, 0.26 mmol) and 2 minutes later acryloyl chloride (6.0 μl, 0.074 mmol). The mixture was stirred for 10 minutes, then quenched with 0.2 ml TFA and diluted with 2 ml water. The crude was purified directly by reverse phase preparative HPLC, using 0.1% formic acid in Water and CH₃CN as mobile phase, to give 3-((1-acryloylpiperidin-4-yl)methylamino)-5-(4-isopropylphenylamino)-1,2,4-triazine-6-carboxamide (6.0 mg, 38%), as a yellow solid, formic acid salt; MW=423.5, MH⁺=424.3, MH⁻=422.2.

### Example C-23: Synthesis of (S)-5-((1-acryloylpiperidin-3-yl)(methyl)amino)-3-(4-isopropylphenylamino)pyrazine-2-carboxamide (23)

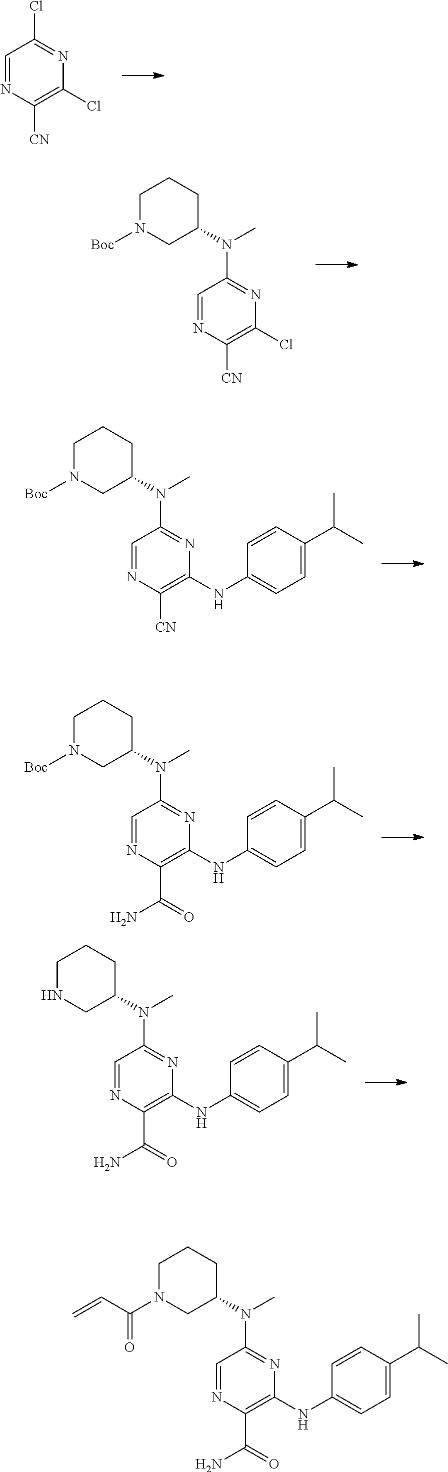

To a solution of 3,5-dichloropyrazine-2-carbonitrile (1035 mg, 5.98 mmol) in DMF (10 mL) were added (S)-tert-butyl 3-(methylamino)piperidine-1-carboxylate (1280 mg, 5.98 mmol) and DIPEA (1.25 mL, 7.2 mmol) in a dropwise manner. The mixture was stirred at room temperature for 3 hrs. The reaction mixture was diluted with 200 mL EtOAc, washed with brine ×3, dried, concentrated in vacuo and subjected to silica flash column with 0 to 60% EtOAc in hexane to give (S)-tert-butyl 3-((6-chloro-5-cyanopyrazin-2-yl)methyl)amino)piperidine-1-carboxylate (1.50 g, 71%).

A mixture of (S)-tert-butyl 3-((6-chloro-5-cyanopyrazin-2-yl)(methyl)amino)piperidine-1-carboxylate (220 mg, 0.63 mmol), 4-isopropylaniline (180 μL, 1.26 mmol), Pd(OAc)₂ (42 mg, 0.19 mmol), BINAP (120 mg, 0.19 mmol), fine powder Cs₂CO₃ (820 mg, 2.52 mmol) in dioxane (40 mL) was degassed with a nitrogen stream for 5 min. The mixture was stirred in a nitrogen atmosphere at 115° C. for 2 hrs. The mixture was cooled to RT, diluted with ethyl acetate, filtered through a disposable ChemGlass filter (cat# OP-6602-12), and concentrated in vacuo. The residue was purified by flash chromatography with 0 to 40% ethyl acetate in hexane to give (S)-tert-butyl 3-((5-cyano-6-(4-isopropylphenylamino)pyrazin-2-yl)(methyl)amino)piperidine-1-carboxylate. It was dissolved in 20 mL MeOH and 5 mL DMSO. To it were added 1 NaOH pellet and then 1 mL 30% H₂O₂. The mixture was stirred at room temperature for 2 hrs, yielding (S)-tert-butyl 3-((5-carbamoyl-6-(4-isopropylphenylamino)pyrazin-2-yl)(methyl)amino)piperidine-1-carboxylate. To the mixture was added 5 mL acetonitrile, and the mixture was concentrated to dryness. To it were added EtOAc and water, and the organic phase was separated, washed, dried, concentrated, and treated with 5 mL TFA for 20 min. The mixture was concentrated, diluted with water and directly subjected to reverse phase preparative HPLC to isolate (S)-3-(4-isopropylphenylamino)-5-(methyl(piperidin-3-yl)amino)pyrazine-2-carboxamide as HCl salt (194 mg). MS found for C20H28N6O as (M+H)⁺ 396.4.

To a solution of (S)-3-(4-isopropylphenylamino)-5-(methyl(piperidin-3-yl)amino)pyrazine-2-carboxamide (HCl salt, 20 mg, 0.049 mmol) in 1.6 mL NMP were added DIPEA (60 μL, 0.35 mmol) and 2 minutes later acryloyl chloride (8.0 μl, 0.10 mmol). The mixture was stirred for 10 minutes, then quenched with 0.2 mL TFA and diluted with 2 mL water. The crude was purified directly by reverse phase preparative HPLC, using 0.1% formic acid in water and neat acetonitrile as mobile phases, to give the title compound (S)-5-((1-acryloylpiperidin-3-yl)(methyl)amino)-3-(4-isopropylphenylamino)pyrazine-2-carboxamide (16 mg), as a yellow solid, formic acid salt. MS found for C₂₃H₃₀N₆O₂ as (M+H)⁺ 423.3.

### Example C-24: Synthesis of 5-(((1-acryloylpiperidin-4-yl)methyl)(methyl)amino)-3-(4-isopropylphenylamino)pyrazine-2-carboxamide (24)

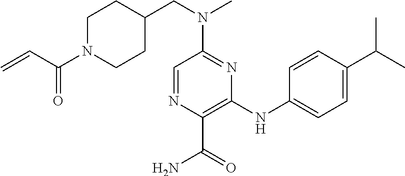 [SMILES: C=CC(=O)N1CCC(CN(C)c2cnc(C(N)=O)c(Nc3ccc(C(C)C)cc3)n2)CC1]

In a similar manner as described in Example C-23, 5-(((1-acryloylpiperidin-4-yl)methyl)(methyl)amino)-3-(4-isopropylphenylamino)pyrazine-2-carboxamide was prepared using tert-butyl 4-((methylamino)methyl)piperidine-1-carboxylate. MS found for C24H32N6O2 as (M+H)⁺ 437.3.

### Example C-25: Synthesis of (S)-5-((1-acryloylpiperidin-3-yl)(methyl)amino)-3-(3-phenylisothiazol-5-ylamino)pyrazine-2-carboxamide (25)

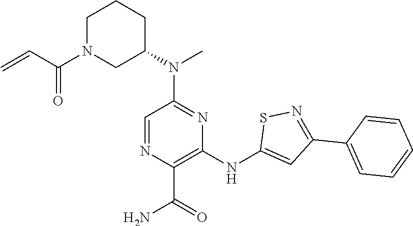 [SMILES: C=CC(=O)N1CCC[C@H](N(C)c2cnc(C(N)=O)c(Nc3cc(-c4ccccc4)ns3)n2)C1]

In a similar manner as described in Example C-23, (S)-5-((1-acryloylpiperidin-3-yl)(methyl)amino)-3-(3-phenylisothiazol-5-ylamino)pyrazine-2-carboxamide was prepared using 5-amino-3-phenylisothiazole. MS found for C23H25N7O2S as (M+H)⁺ 464.4, and (M−H)⁺ 462.2.

### Example C-26: Synthesis of (R)-3-(1-acryloylpiperidin-3-ylamino)-5-(4-isopropylphenylamino)-1,2,4-triazine-6-carboxamide (26)

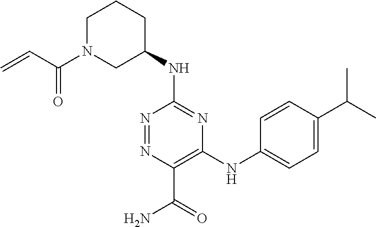 [SMILES: C=CC(=O)N1CCC[C@@H](Nc2nnc(C(N)=O)c(Nc3ccc(C(C)C)cc3)n2)C1]

In a similar manner as described in Example C-17, (R)-3-(1-acryloylpiperidin-3-ylamino)-5-(4-isopropylphenylamino)-1,2,4-triazine-6-carboxamide was prepared using (R)-tert-butyl 3-aminopiperidine-1-carboxylate. MS found for C21H27N7O2 as (M+H)⁺ 410.3, and (M−H)⁻ 408.1.

### Example C-27: Synthesis of (R)-3-((1-acryloylpiperidin-3-yl)(methyl)amino)-5-(4-isopropylphenylamino)-1,2,4-triazine-6-carboxamide (27)

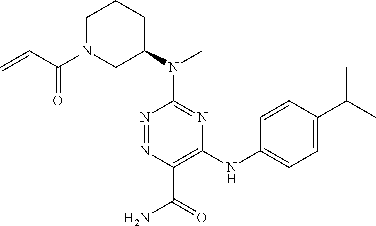 [SMILES: C=CC(=O)N1CCC[C@@H](N(C)c2nnc(C(N)=O)c(Nc3ccc(C(C)C)cc3)n2)C1]

In a similar manner as described in Example C-23, (R)-3-((1-acryloylpiperidin-3-yl)(methyl)amino)-5-(4-isopropylphenylamino)-1,2,4-triazine-6-carboxamide was prepared using (R)-tert-butyl 3-(methylamino)piperidine-1-carboxylate. MS found for C22H29N7O2 as (M+H)⁺ 424.4.

### Example C-28: Synthesis of 3-((2R,3R)-1-acryloyl-2-methylpiperidin-3-ylamino)-5-(4-isopropylphenylamino)-1,2,4-triazine-6-carboxamide (28)

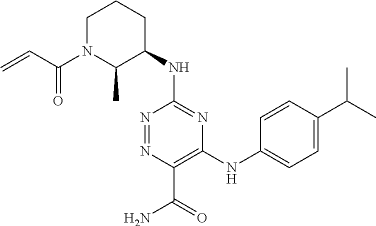 [SMILES: C=CC(=O)N1CCC[C@@H](Nc2nnc(C(N)=O)c(Nc3ccc(C(C)C)cc3)n2)[C@H]1C]

In a similar manner as described in Example C-17, (S)-5-((1-acryloylpiperidin-3-yl)(methyl)amino)-3-(3-phenylisothiazol-5-ylamino)pyrazine-2-carboxamide was prepared using (2R,3R)-tert-butyl 3-amino-2-methylpiperidine-1-carboxylate. MS found for C22H29N7O2 as (M+H)⁺ 424.4, and (M−H)⁻ 422.2.

### Examples C-29-C-89

Compounds in Table 4 were prepared using a method similar to those described herein or methods known in the art.

TABLE 4
Compounds of Formula (C-I)

| Cmpd. No. | Compound Structure | Compound Name | MS |
|---|---|---|---|
| C-29 | 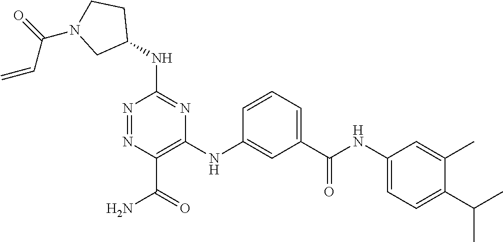 [SMILES: C=CC(=O)N1CC[C@H](Nc2nnc(C(N)=O)c(Nc3cccc(C(=O)Nc4ccc(C(C)C)c(C)c4)c3)n2)C1] | (S)-3-(1- acryloylpyrrolidin-3- ylamino)-5-(3-(4- isopropyl-3- methylphenyl- carbamoyl)phenyl- amino)-1,2,4- triazine-6-carboxamide | |
| C-30 | 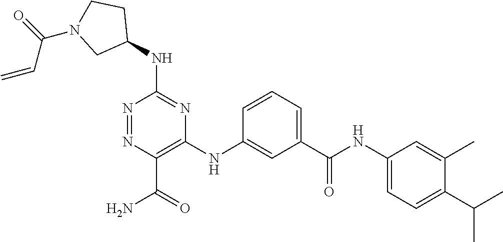 [SMILES: C=CC(=O)N1CC[C@@H](Nc2nnc(C(N)=O)c(Nc3cccc(C(=O)Nc4ccc(C(C)C)c(C)c4)c3)n2)C1] | (R)-3-(1- acryloylpyrrolidin-3- ylamino)-5-(3-(4- isopropyl-3- methylphenyl- carbamoyl)phenyl- amino)-1,2,4- triazine-6-carboxamide | |
| C-31 | 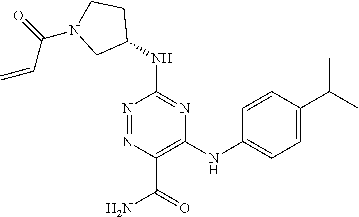 [SMILES: C=CC(=O)N1CC[C@H](Nc2nnc(C(N)=O)c(Nc3ccc(C(C)C)cc3)n2)C1] | (S)-3-(1- acryloylpyrrolidin-3- ylamino)-5-(4- isopropylphenylamino)- 1,2,4-triazine-6- carboxamide | |
| C-32 | 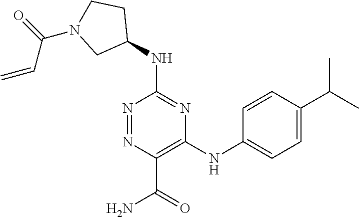 [SMILES: C=CC(=O)N1CC[C@@H](Nc2nnc(C(N)=O)c(Nc3ccc(C(C)C)cc3)n2)C1] | (R)-3-(1- acryloylpyrrolidin-3- ylamino)-5-(4- isopropylphenylamino)- 1,2,4-triazine-6- carboxamide | |
| C-33 | 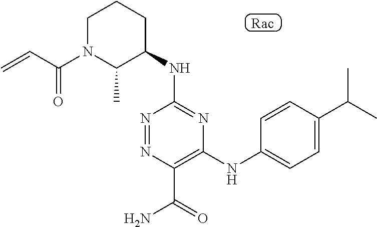 [SMILES: C=CC(=O)N1CCC[C@@H](Nc2nnc(C(N)=O)c(Nc3ccc(C(C)C)cc3)n2)[C@@H]1C ; "Rac"] | 3-((2S,3R)-1-acryloyl- 2-methylpiperidin-3- ylamino)-5-(4- isopropylphenylamino)- 1,2,4-triazine-6- carboxamide (racemic) | M + H = 424.3; M − H = 422.2 |
| C-34 | 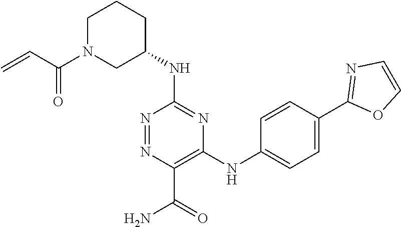 [SMILES: C=CC(=O)N1CCC[C@H](Nc2nnc(C(N)=O)c(Nc3ccc(-c4ncco4)cc3)n2)C1] | (S)-3-(1- acryloylpiperidin-3- ylamino)-5-(4-(oxazol- 2-yl)phenylamino)- 1,2,4-triazine-6- carboxamide | M + H = 435.2; M − H = 433.1 |
| C-35 | 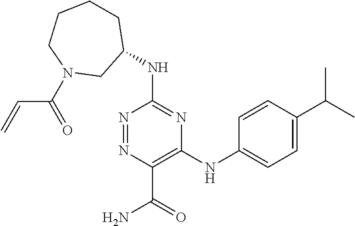 [SMILES: C=CC(=O)N1CCCC[C@H](Nc2nnc(C(N)=O)c(Nc3ccc(C(C)C)cc3)n2)C1] | (S)-3-(1- acryloylazepan-3- ylamino)-5-(4- isopropylphenylamino)- 1,2,4-triazine-6- carboxamide | M + H = 424.4; M − H = 422.2 |
| C-36 | 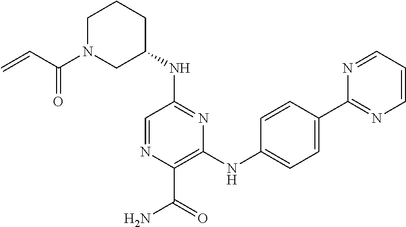 [SMILES: C=CC(=O)N1CCC[C@H](Nc2cnc(C(N)=O)c(Nc3ccc(-c4ncccn4)cc3)n2)C1] | (S)-5-(1- acryloylpiperidin-3- ylamino)-3-(4- (pyrimidin-2- yl)phenylamino) pyrazine-2-carboxamide | M + H = 445.2; M − H = 443.1 |
| C-37 | 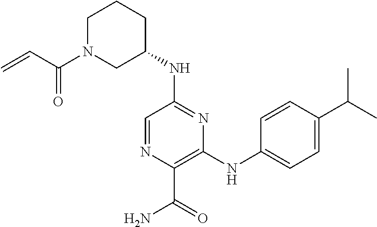 [SMILES: C=CC(=O)N1CCC[C@H](Nc2cnc(C(N)=O)c(Nc3ccc(C(C)C)cc3)n2)C1] | (S)-5-(1- acryloylpiperidin-3- ylamino)-3-(4- isopropylphenylamino) pyrazine-2- carboxamide | M + H = 409.1; M − H = 407.1 |
| C-38 | 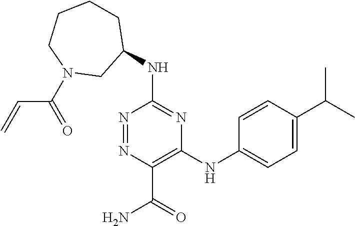 [SMILES: C=CC(=O)N1CCCC[C@@H](Nc2nnc(C(N)=O)c(Nc3ccc(C(C)C)cc3)n2)C1] | (R)-3-(1- acryloylazepan-3- ylamino)-5-(4- isopropylphenylamino)- 1,2,4-triazine-6- carboxamide | M + H = 424.2; M − H = 422.2 |
| C-39 | 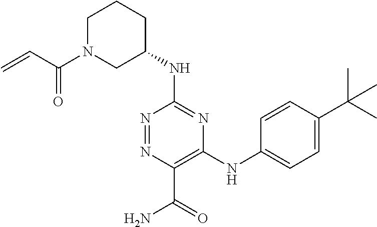 [SMILES: C=CC(=O)N1CCC[C@H](Nc2nnc(C(N)=O)c(Nc3ccc(C(C)(C)C)cc3)n2)C1] | (S)-3-(1- acryloylpiperidin-3- ylamino)-5-(4-tert- butylphenylamino)- 1,2,4-triazine-6- carboxamide | M + H = 424.4; M − H = 422.1 |
| C-40 | 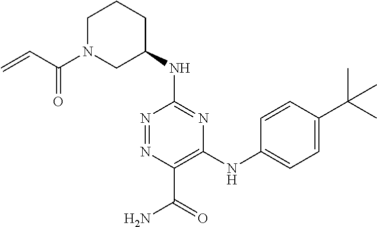 [SMILES: C=CC(=O)N1CCC[C@@H](Nc2nnc(C(N)=O)c(Nc3ccc(C(C)(C)C)cc3)n2)C1] | (R)-3-(1- acryloylpiperidin-3- ylamino)-5-(4-tert- butylphenylamino)- 1,2,4-triazine-6- carboxamide | M + H = 424.4; M − H = 422.1 |
| C-41 | 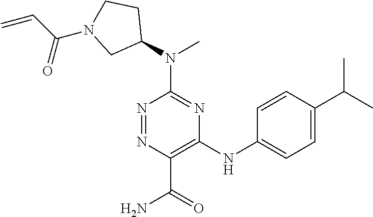 [SMILES: C=CC(=O)N1CC[C@@H](N(C)c2nnc(C(N)=O)c(Nc3ccc(C(C)C)cc3)n2)C1] | (R)-3-((1- acryloylpyrrolidin-3- yl)(methyl)amino)-5- (4- isopropylphenylamino)- 1,2,4-triazine-6- carboxamide | M + H = 410.4; M − H = 408.1 |
| C-42 | 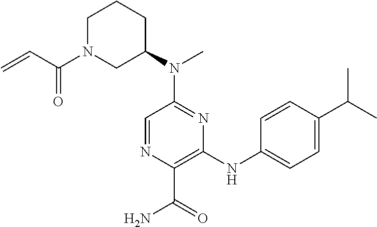 [SMILES: C=CC(=O)N1CCC[C@@H](N(C)c2cnc(C(N)=O)c(Nc3ccc(C(C)C)cc3)n2)C1] | (R)-5-((1- acryloylpiperidin-3- yl)(methyl)amino)-3- (4- isopropylphenylamino) pyrazine-2- carboxamide | M + H = 423.2; |
| C-43 | 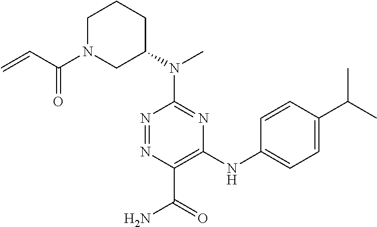 [SMILES: C=CC(=O)N1CCC[C@H](N(C)c2nnc(C(N)=O)c(Nc3ccc(C(C)C)cc3)n2)C1] | (S)-3-((1- acryloylpiperidin-3- yl)(methyl)amino)-5- (4- isopropylphenylamino)- 1,2,4-triazine-6- carboxamide | M + H = 424.3; M − H = 422.1 |
| C-44 | 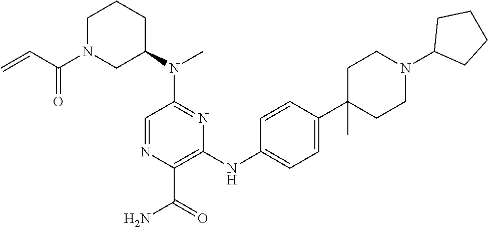 [SMILES: C=CC(=O)N1CCC[C@@H](N(C)c2cnc(C(N)=O)c(Nc3ccc(C4(C)CCN(C5CCCC5)CC4)cc3)n2)C1] | (R)-5-((1- acryloylpiperidin-3- yl)(methyl)amino)-3- (4-(1-cyclopentyl-4- methylpiperidin-4- yl)phenylamino) pyrazine-2-carboxamide | M + H = 546.7 |
| C-45 | 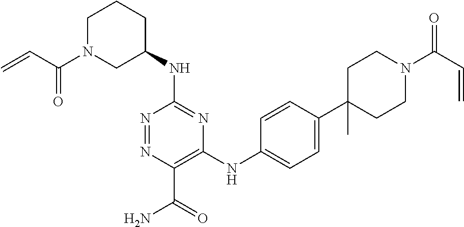 [SMILES: C=CC(=O)N1CCC(C)(c2ccc(Nc3nc(N[C@@H]4CCCN(C(=O)C=C)C4)nnc3C(N)=O)cc2)CC1] | (R)-5-(4-(1-acryloyl-4- methylpiperidin-4- yl)phenylamino)-3-(1- acryloylpiperidin-3- ylamino)-1,2,4-triazine- 6-carboxamide | M + H = 519.4; M − H = 517.2 |
| C-46 | 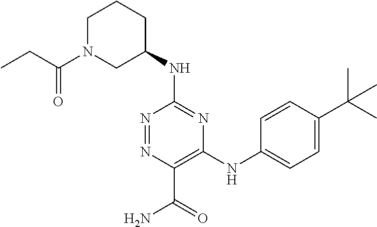 [SMILES: CCC(=O)N1CCC[C@@H](Nc2nnc(C(N)=O)c(Nc3ccc(C(C)(C)C)cc3)n2)C1] | (R)-5-(4-tert- butylphenylamino)-3- (1-propionylpiperidin- 3-ylamino)-1,2,4- triazine-6-carboxamide | M + H = 426.5; M − H = 424.1 |
| C-47 | 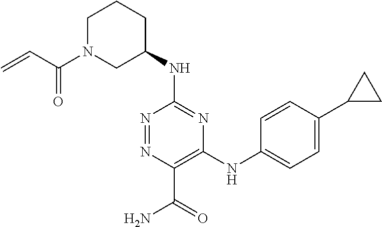 [SMILES: C=CC(=O)N1CCC[C@@H](Nc2nnc(C(N)=O)c(Nc3ccc(C4CC4)cc3)n2)C1] | (R)-3-(1- acryloylpiperidin-3- ylamino)-5-(4- cyclopropylphenyl- amino)-1,2,4-triazine- 6-carboxamide | M⁺H = 408.2; M⁻H = 406.1 |
| C-48 | 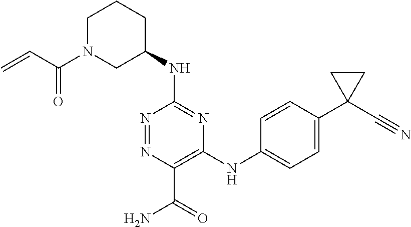 [SMILES: C=CC(=O)N1CCC[C@@H](Nc2nnc(C(N)=O)c(Nc3ccc(C4(C#N)CC4)cc3)n2)C1] | (R)-3-(1- acryloylpiperidin-3- ylamino)-5-(4-(1- cyanocyclopropyl) phenylamino)-1,2,4- triazine-6-carboxamide | M⁺H = 433.1; M⁻H = 431.1 |
| C-49 | 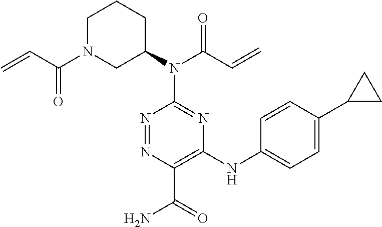 [SMILES: C=CC(=O)N1CCC[C@@H](N(C(=O)C=C)c2nnc(C(N)=O)c(Nc3ccc(C4CC4)cc3)n2)C1] | (R)-3-(N-(1- acryloylpiperidin-3- yl)acrylamido)-5-(4- cyclopropylphenyl- amino)-1,2,4-triazine- 6-carboxamide | M⁺H = 462.4; M⁻H = 460.1 |
| C-50 | 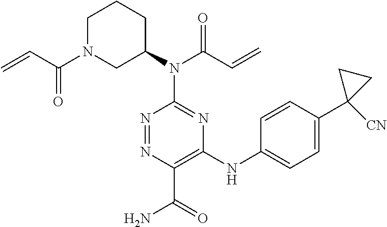 [SMILES: C=CC(=O)N1CCC[C@@H](N(C(=O)C=C)c2nnc(C(N)=O)c(Nc3ccc(C4(C#N)CC4)cc3)n2)C1] | (R)-3-(N-(1- acryloylpiperidin-3- yl)acrylamido)-5-(4-(1- cyanocyclopropyl) phenylamino)-1,2,4- triazine-6-carboxamide | M⁺H = 487.3; M⁻H = 485.1 |
| C-51 | 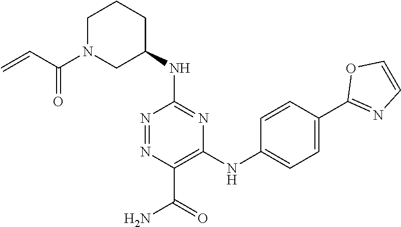 [SMILES: C=CC(=O)N1CCC[C@@H](Nc2nnc(C(N)=O)c(Nc3ccc(-c4ncco4)cc3)n2)C1] | (R)-3-(1- acryloylpiperidin-3- ylamino)-5-(4-(oxazol- 2-yl)phenylamino)- 1,2,4-triazine-6- carboxamide | M + H = 435.3; M − H = 433.1 |
| C-52 | 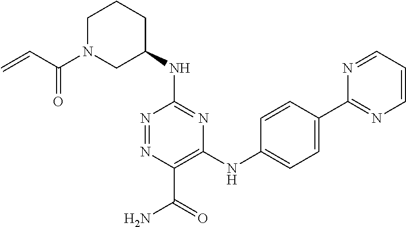 [SMILES: C=CC(=O)N1CCC[C@@H](Nc2nnc(C(N)=O)c(Nc3ccc(-c4ncccn4)cc3)n2)C1] | (R)-3-(1- acryloylpiperidin-3- ylamino)-5-(4- (pyrimidin-2- yl)phenylamino)-1,2,4- triazine-6-carboxamide | M + H = 446.2; M − H = 444.1 |
| C-53 | 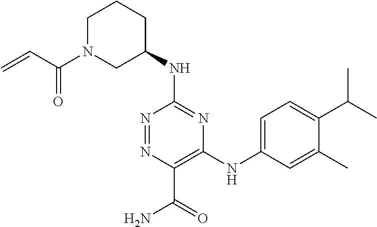 [SMILES: C=CC(=O)N1CCC[C@@H](Nc2nnc(C(N)=O)c(Nc3ccc(C(C)C)c(C)c3)n2)C1] | (R)-3-(1- acryloylpiperidin-3- ylamino)-5-(4- isopropyl-3- methylphenylamino)- 1,2,4-triazine-6- carboxamide | M⁺H = 424.3; M⁻H = 422.1 |
| C-54 | 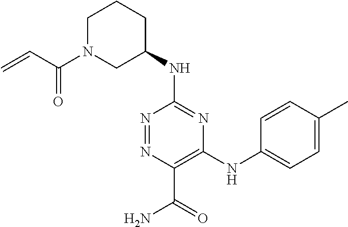 [SMILES: C=CC(=O)N1CCC[C@@H](Nc2nnc(C(N)=O)c(Nc3ccc(C)cc3)n2)C1] | (R)-3-(1- acryloylpiperidin-3- ylamino)-5-(p- tolylamino)-1,2,4- triazine-6-carboxamide | M⁺H = 382.3; M⁻H = 380.1 |
| C-55 | 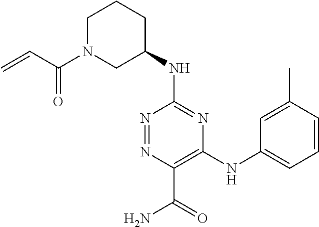 [SMILES: C=CC(=O)N1CCC[C@@H](Nc2nnc(C(N)=O)c(Nc3cccc(C)c3)n2)C1] | (R)-3-(1- acryloylpiperidin-3- ylamino)-5-(m- tolylamino)-1,2,4- triazine-6-carboxamide | M⁺H = 382.1; M⁻H = 380.1 |
| C-56 | 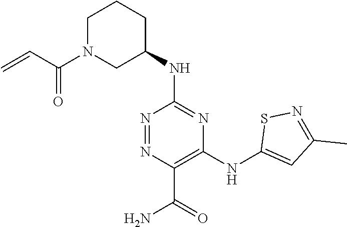 [SMILES: C=CC(=O)N1CCC[C@@H](Nc2nnc(C(N)=O)c(Nc3cc(C)ns3)n2)C1] | (R)-3-(1- acryloylpiperidin-3- ylamino)-5-(3- methylisothiazol-5- ylamino)-1,2,4-triazine- 6-carboxamide | M + H = 389.3; M − H = 387.1 |
| C-57 | 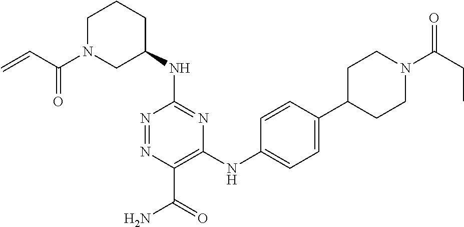 [SMILES: C=CC(=O)N1CCC[C@@H](Nc2nnc(C(N)=O)c(Nc3ccc(C4CCN(C(=O)CC)CC4)cc3)n2)C1] | (R)-3-(1- acryloylpiperidin-3- ylamino)-5-(4-(1- propionylpiperidin-4- yl)phenylamino)-1,2,4- triazine-6-carboxamide | M + H = 507.4; M − H = 505.2 |
| C-58 | 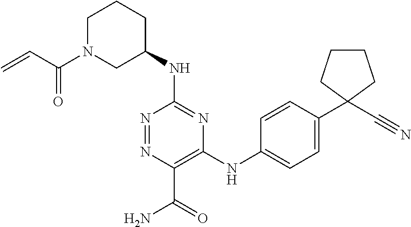 [SMILES: C=CC(=O)N1CCC[C@@H](Nc2nnc(C(N)=O)c(Nc3ccc(C4(C#N)CCCC4)cc3)n2)C1] | (R)-3-(1- acryloylpiperidin-3- ylamino)-5-(4-(1- cyanocyclopentyl) phenylamino)-1,2,4- triazine-6-carboxamide | M + H = 461.4; M − H = 459.1 |
| C-59 | 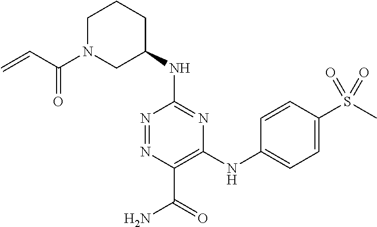 [SMILES: C=CC(=O)N1CCC[C@@H](Nc2nnc(C(N)=O)c(Nc3ccc(S(C)(=O)=O)cc3)n2)C1] | (R)-3-(1- acryloylpiperidin-3- ylamino)-5-(4- (methylsulfonyl)phenyl- amino)-1,2,4-triazine- 6-carboxamide | M + H = 446.1; M − H = 444.0 |
| C-60 | 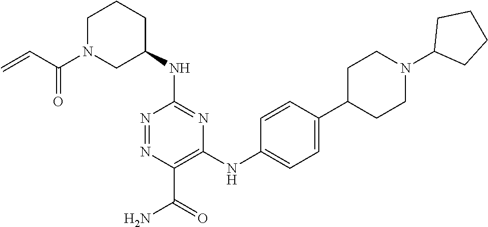 [SMILES: C=CC(=O)N1CCC[C@@H](Nc2nnc(C(N)=O)c(Nc3ccc(C4CCN(C5CCCC5)CC4)cc3)n2)C1] | (R)-3-(1- acryloylpiperidin-3- ylamino)-5-(4-(1- cyclopentylpiperidin-4- yl)phenylamino)-1,2,4- triazine-6-carboxamide | M + H = 519.4; M − H = 517.3 |
| C-61 | 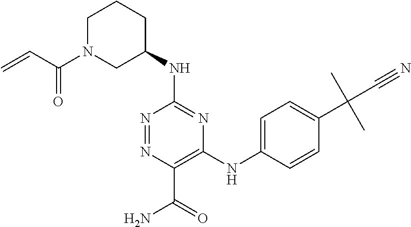 [SMILES: C=CC(=O)N1CCC[C@@H](Nc2nnc(C(N)=O)c(Nc3ccc(C(C)(C)C#N)cc3)n2)C1] | (R)-3-(1- acryloylpiperidin-3- ylamino)-5-(4-(2- cyanopropan-2- yl)phenylamino)-1,2,4- triazine-6-carboxamide | M⁺H = 435.3; M⁻H = 433.1 |
| C-62 | 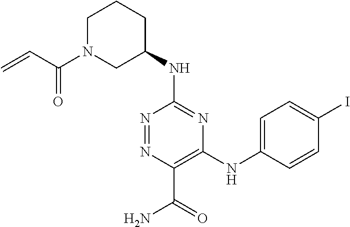 [SMILES: C=CC(=O)N1CCC[C@@H](Nc2nnc(C(N)=O)c(Nc3ccc(I)cc3)n2)C1] | (R)-3-(1- acryloylpiperidin-3- ylamino)-5-(4- iodophenylamino)- 1,2,4-triazine-6- carboxamide | M⁺H = 494.0; M⁻H = 492.0 |
| C-63 | 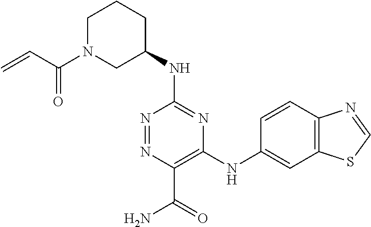 [SMILES: C=CC(=O)N1CCC[C@@H](Nc2nnc(C(N)=O)c(Nc3ccc4ncsc4c3)n2)C1] | (R)-3-(1- acryloylpiperidin-3- ylamino)-5- (benzo[d]thiazol-6- ylamino)-1,2,4-triazine- 6-carboxamide | M + H = 425.0; M − H = 423.0 |
| C-64 | 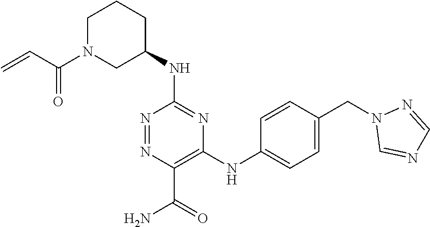 [SMILES: C=CC(=O)N1CCC[C@@H](Nc2nnc(C(N)=O)c(Nc3ccc(Cn4cncn4)cc3)n2)C1] | (R)-5-(4-((1H-1,2,4- triazol-1- yl)methyl)phenyl- amino)-3-(1- acryloylpiperidin-3- ylamino)-1,2,4-triazine- 6-carboxamide | M + H = 449.1; M − H = 447.1 |
| C-65 | 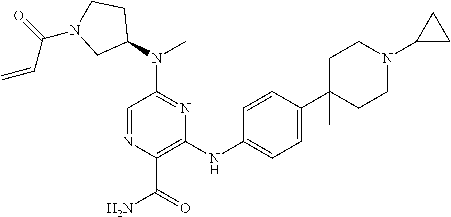 [SMILES: C=CC(=O)N1CC[C@@H](N(C)c2cnc(C(N)=O)c(Nc3ccc(C4(C)CCN(C5CC5)CC4)cc3)n2)C1] | (R)-5-((1- acryloylpyrrolidin-3- yl)(methyl)amino)-3- (4-(1-cyclopropyl-4- methylpiperidin-4- yl)phenylamino) pyrazine-2-carboxamide | M + H = 504.5; M − H = 502.2 |
| C-66 | 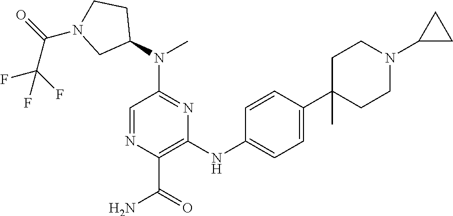 [SMILES: CN(c1cnc(C(N)=O)c(Nc2ccc(C3(C)CCN(C4CC4)CC3)cc2)n1)[C@@H]1CCN(C(=O)C(F)(F)F)C1] | (R)-3-(4-(1- cyclopropyl-4- methylpiperidin-4- yl)phenylamino)-5- (methyl(1-(2,2,2- trifluoroacetyl)pyrrolidin- 3-yl)amino)pyrazine- 2-carboxamide | M + H = 546.5; M − H = 544.2 |
| C-67 | 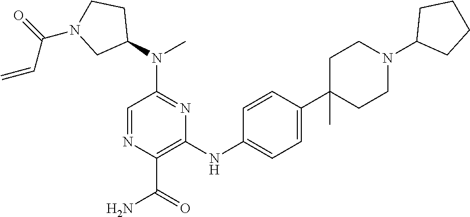 [SMILES: C=CC(=O)N1CC[C@@H](N(C)c2cnc(C(N)=O)c(Nc3ccc(C4(C)CCN(C5CCCC5)CC4)cc3)n2)C1] | (R)-5-((1- acryloylpyrrolidin-3- yl)(methyl)amino)-3- (4-(1-cyclopentyl-4- methylpiperidin-4- yl)phenylamino)pyrazine- 2-carboxamide | M + H = 532.7; M − H = 530.3 |
| C-68 | 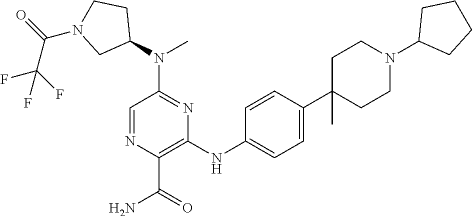 [SMILES: CN(c1cnc(C(N)=O)c(Nc2ccc(C3(C)CCN(C4CCCC4)CC3)cc2)n1)[C@@H]1CCN(C(=O)C(F)(F)F)C1] | (R)-3-(4-(1- cyclopentyl-4- methylpiperidin-4- yl)phenylamino)-5- (methyl(1-(2,2,2- trifluoroacetyl)pyrrolidin- 3-yl)amino)pyrazine- 2-carboxamide | M + H = 574.5; M − H = 572.3 |
| C-69 | 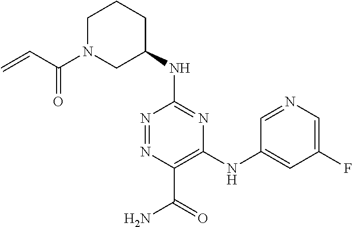 [SMILES: C=CC(=O)N1CCC[C@@H](Nc2nnc(C(N)=O)c(Nc3cncc(F)c3)n2)C1] | (R)-3-(1- acryloylpiperidin-3- ylamino)-5-(5- fluoropyridin-3- ylamino)-1,2,4-triazine- 6-carboxamide | M + H = 387.0; M − H = 385.1 |
| C-70 | 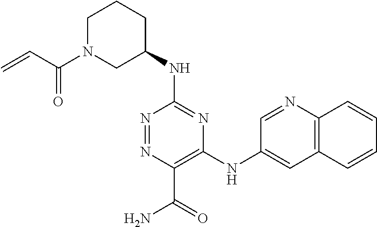 [SMILES: C=CC(=O)N1CCC[C@@H](Nc2nnc(C(N)=O)c(Nc3cnc4ccccc4c3)n2)C1] | (R)-3-(1- acryloylpiperidin-3- ylamino)-5-(quinolin-3- ylamino)-1,2,4-triazine- 6-carboxamide | M + H = 419.2; M − H = 417.1 |
| C-71 | 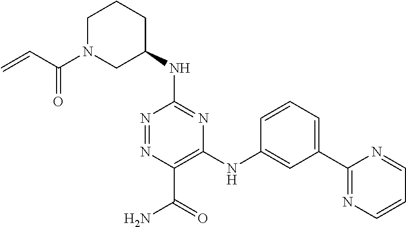 [SMILES: C=CC(=O)N1CCC[C@@H](Nc2nnc(C(N)=O)c(Nc3cccc(-c4ncccn4)c3)n2)C1] | (R)-3-(1- acryloylpiperidin-3- ylamino)-5-(3- (pyrimidin-2- yl)phenylamino)-1,2,4- triazine-6-carboxamide | M + H = 446.2; M − H = 444.1 |
| C-72 | 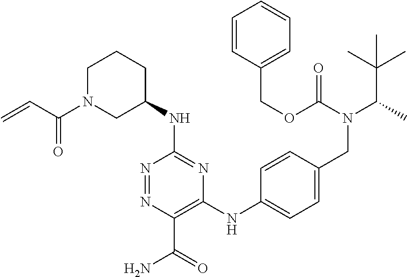 [SMILES: C=CC(=O)N1CCC[C@@H](Nc2nnc(C(N)=O)c(Nc3ccc(CN(C(=O)OCc4ccccc4)[C@@H](C)C(C)(C)C)cc3)n2)C1] | benzyl 4-(3-((R)-1- acryloylpiperidin-3- ylamino)-6-carbamoyl- 1,2,4-triazin-5- ylamino)benzyl((S)- 3,3-dimethylbutan-2- yl)carbamate | M + H = 615.6; M − H = 613.3 |
| C-73 | 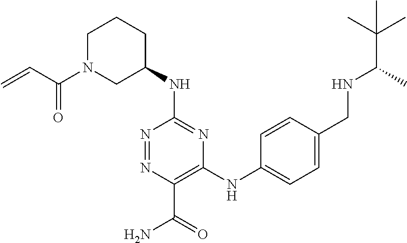 [SMILES: C=CC(=O)N1CCC[C@@H](Nc2nnc(C(N)=O)c(Nc3ccc(CN[C@@H](C)C(C)(C)C)cc3)n2)C1] | 3-((R)-1- acryloylpiperidin-3- ylamino)-5-(4-(((S)- 3,3-dimethylbutan-2- ylamino)methyl)phenyl- amino)-1,2,4-triazine- 6-carboxamide | M + H = 481.3; M − H = 479.2 |
| C-74 | 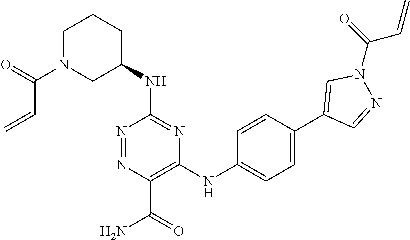 [SMILES: C=CC(=O)N1CCC[C@@H](Nc2nnc(C(N)=O)c(Nc3ccc(-c4cnn(C(=O)C=C)c4)cc3)n2)C1] | (R)-5-(4-1-acryloyl- 1H-pyrazol-4- yl)phenylamino)-3-(1- acryloylpiperidin-3- ylamino)-1,2,4-triazine- 6-carboxamide | M + H = 488.2; M − H = 486.1 |
| C-75 | 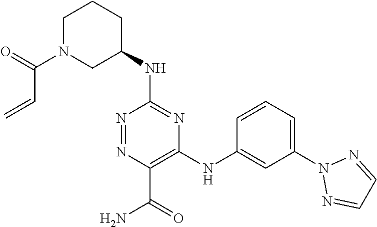 [SMILES: C=CC(=O)N1CCC[C@@H](Nc2nnc(C(N)=O)c(Nc3cccc(-n4nccn4)c3)n2)C1] | (R)-5-(3-(2H-1,2,3- triazol-2- yl)phenylamino)-3-(1- acryloylpiperidin-3- ylamino)-1,2,4-triazine- 6-carboxamide | M + H = 435.3; M − H = 433.1 |
| C-76 | 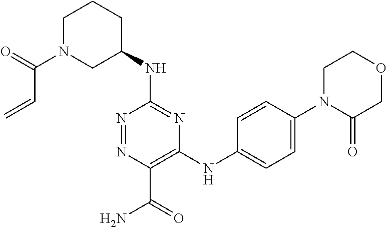 [SMILES: C=CC(=O)N1CCC[C@@H](Nc2nnc(C(N)=O)c(Nc3ccc(N4CCOCC4=O)cc3)n2)C1] | (R)-3-(1- acryloylpiperidin-3- ylamino)-5-(4-(3- oxomorpholino)phenyl- amino)-1,2,4-triazine- 6-carboxamide | M + H = 467.2; M − H = 465.1 |
| C-77 | 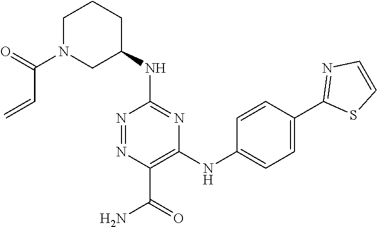 [SMILES: C=CC(=O)N1CCC[C@@H](Nc2nnc(C(N)=O)c(Nc3ccc(-c4nccs4)cc3)n2)C1] | (R)-3-(1- acryloylpiperidin-3- ylamino)-5-(4-(thiazol- 2-yl)phenylamino)- 1,2,4-triazine-6- carboxamide | M + H = 451.2; M − H = 449.0 |
| C-78 | 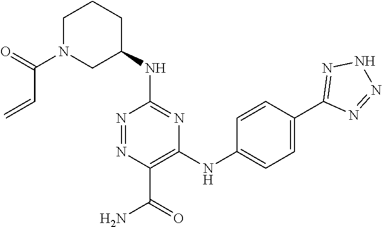 [SMILES: C=CC(=O)N1CCC[C@@H](Nc2nnc(C(N)=O)c(Nc3ccc(-c4nn[nH]n4)cc3)n2)C1] | (R)-5-(4-(2H-tetrazol- 5-yl)phenylamino)-3- (1-acryloylpiperidin-3- ylamino)-1,2,4-triazine- 6-carboxamide | M + H = 436.1; M − H = 434.1 |
| C-79 | 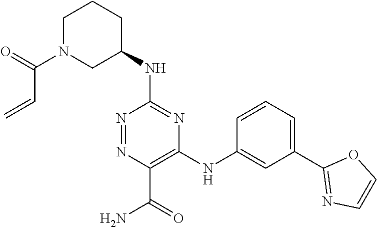 [SMILES: C=CC(=O)N1CCC[C@@H](Nc2nnc(C(N)=O)c(Nc3cccc(-c4ncco4)c3)n2)C1] | (R)-3-(1- acryloylpiperidin-3- ylamino)-5-(3-(oxazol- 2-yl)phenylamino)- 1,2,4-triazine-6- carboxamide | M + H = 435.3; M − H = 433.1 |
| C-80 | 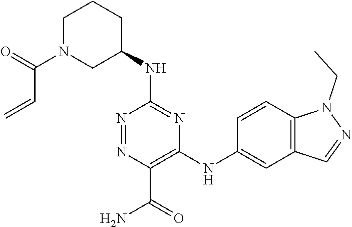 [SMILES: C=CC(=O)N1CCC[C@@H](Nc2nnc(C(N)=O)c(Nc3ccc4c(cnn4CC)c3)n2)C1] | (R)-3-(1- acryloylpiperidin-3- ylamino)-5-(1-ethyl- 1H-indazol-5-ylamino)- 1,2,4-triazine-6- carboxamide | M + H = 436.3; M − H = 434.1 |
| C-81 | 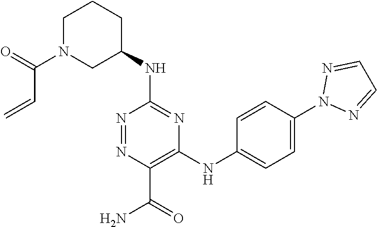 [SMILES: C=CC(=O)N1CCC[C@@H](Nc2nnc(C(N)=O)c(Nc3ccc(-n4nccn4)cc3)n2)C1] | (R)-5-(4-(2H-1,2,3- triazol-2- yl)phenylamino)-3-(1- acryloylpiperidin-3- ylamino)-1,2,4-triazine- 6-carboxamide | M + H = 435.2; M − H = 433.1 |
| C-82 | 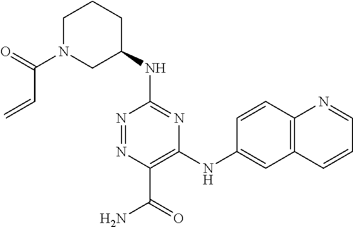 [SMILES: C=CC(=O)N1CCC[C@@H](Nc2nnc(C(N)=O)c(Nc3ccc4ncccc4c3)n2)C1] | (R)-3-(1- acryloylpiperidin-3- ylamino)-5-(quinolin-6- ylamino)-1,2,4-triazine- 6-carboxamide | M + H = 419.1; M − H = 417.1 |
| C-83 | 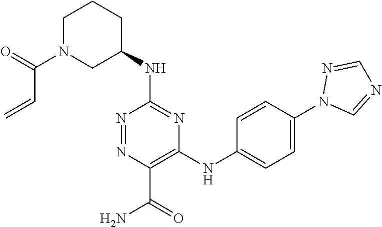 [SMILES: C=CC(=O)N1CCC[C@@H](Nc2nnc(C(N)=O)c(Nc3ccc(-n4cncn4)cc3)n2)C1] | (R)-5-(4-(1H-1,2,4- triazol-1- yl)phenylamino)-3-(1- acryloylpiperidin-3- ylamino)-1,2,4-triazine- 6-carboxamide | M + H = 435.1; M − H = 433.1 |
| C-84 | 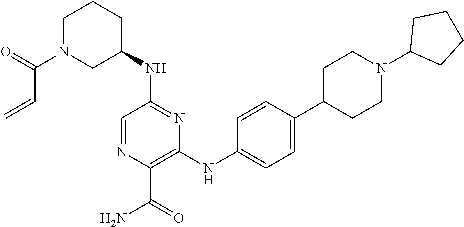 [SMILES: C=CC(=O)N1CCC[C@@H](Nc2cnc(C(N)=O)c(Nc3ccc(C4CCN(C5CCCC5)CC4)cc3)n2)C1] | (R)-5-(1- acryloylpiperidin-3- ylamino)-3-(4-(1- cyclopentylpiperidin-4- yl)phenylamino)pyrazine- 2-carboxamide | M + H = 518.5; M − H = 516.2 |
| C-85 | 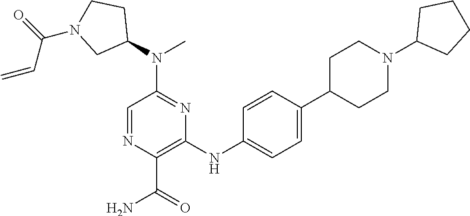 [SMILES: C=CC(=O)N1CC[C@@H](N(C)c2cnc(C(N)=O)c(Nc3ccc(C4CCN(C5CCCC5)CC4)cc3)n2)C1] | (R)-5-((1- acryloylpyrrolidin-3- yl)(methyl)amino)-3- (4-(1- cyclopentylpiperidin-4- yl)phenylamino) pyrazine-2-carboxamide | M + H = 518.6; M − H = 516.3 |
| C-86 | 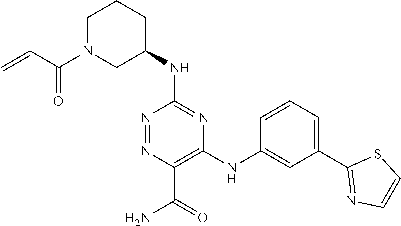 [SMILES: C=CC(=O)N1CCC[C@@H](Nc2nnc(C(N)=O)c(Nc3cccc(-c4nccs4)c3)n2)C1] | (R)-3-(1- acryloylpiperidin-3- ylamino)-5-(3-(thiazol- 2-yl)phenylamino)- 1,2,4-triazine-6- carboxamide | M + H = 451.3; M − H = 449.0 |
| C-87 | 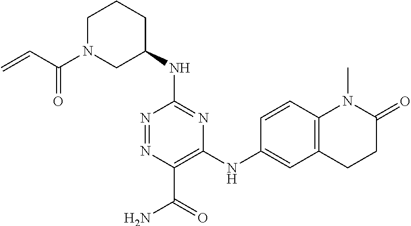 [SMILES: C=CC(=O)N1CCC[C@@H](Nc2nnc(C(N)=O)c(Nc3ccc4c(c3)CCC(=O)N4C)n2)C1] | (R)-3-(1- acryloylpiperidin-3- ylamino)-5-(1-methyl- 2-oxo-1,2,3,4- tetrahydroquinolin-6- ylamino)-1,2,4-triazine- 6-carboxamide | M + H = 451.3; M − H = 449.1 |
| C-88 | 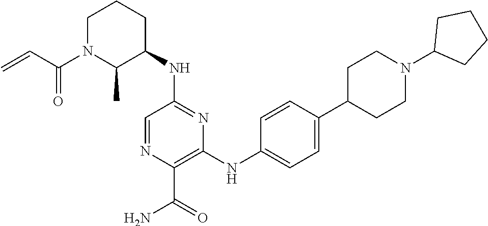 [SMILES: C=CC(=O)N1CCC[C@@H](Nc2cnc(C(N)=O)c(Nc3ccc(C4CCN(C5CCCC5)CC4)cc3)n2)[C@H]1C] | 5-((2R,3R)-1-acryloyl- 2-methylpiperidin-3- ylamino)-3-(4-(1- cyclopentylpiperidin-4- yl)phenylamino) pyrazine-2-carboxamide | M + H = 532.6; M − H = 530.4 |
| C-89 | 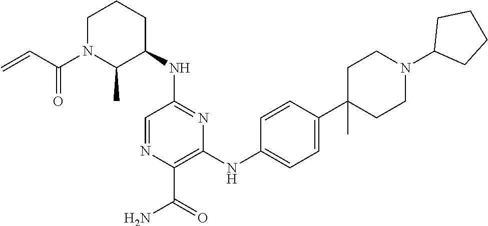 [SMILES: C=CC(=O)N1CCC[C@@H](Nc2cnc(C(N)=O)c(Nc3ccc(C4(C)CCN(C5CCCC5)CC4)cc3)n2)[C@H]1C] | 5-((2R,3R)-1-acryloyl- 2-methylpiperidin-3- ylamino)-3-(4-(1- cyclopentyl-4- methylpiperidin-4- yl)phenylamino) pyrazine-2-carboxamide | M + H = 546.7; M − H = 544.3 |

### Example D-1: Synthesis of 5-[(2R,3R)-3-(4-cyclopropyl-2-fluorobenzamido)-2-methylpiperidin-1-yl]-3-[(1-methyl-1H-pyrazol-4-yl)amino]pyrazine-2-carboxamide (D-1)

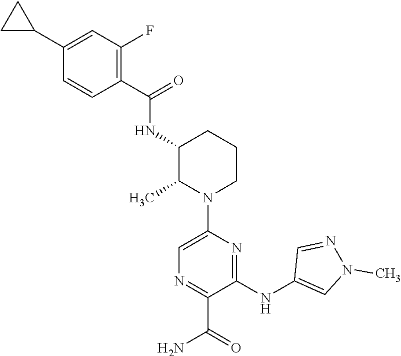 [SMILES: C[C@@H]1[C@H](NC(=O)c2ccc(C3CC3)cc2F)CCCN1c1cnc(C(N)=O)c(Nc2cnn(C)c2)n1]

In a similar manner as described in Example 40, 5-[(2R,3R)-3-(4-cyclopropyl-2-fluoro-benzamido)-2-methylpiperidin-1-yl]-3-[(1-methyl-1H-pyrazol-4-yl)amino]pyrazine-2-carboxamide (D-1) was prepared. MS found for C25H29FN8O2 as (M+H)⁺ 493.4.

¹H NMR (500 MHz, DMSO) δ 10.88 (s, 1H), 8.34 (d, J=6.72 Hz, 1H), 7.69 (d, J=1.92 Hz, 1H), 8.02 (s, 1H), 7.51-7.42 (m, 2H), 7.56 (s, 1H), 7.28 (d, J=1.92 Hz, 1H), 7.05-6.97 (m, 2H), 5.31 (br. s., 1H), 4.18-4.04 (m, 1H), 4.03-3.94 (m, 1H), 3.74 (s, 3H), 3.12-3.02 (m, 1H), 2.05-1.95 (m, 1H), 1.91-1.78 (m, 2H), 1.74-1.49 (m, 2H), 1.11 (d, J=7.00 Hz, 3H), 1.06-0.99 (m, 2H), 0.79-0.72 (m, 2H).

### Example D-2: Synthesis of 5-[(3R)-3-(4-cyclopropyl-2-fluorobenzamido)piperidin-1-yl]-3-[(3-methyl-1,2-oxazol-5-yl)amino]pyrazine-2-carboxamide (D-2)

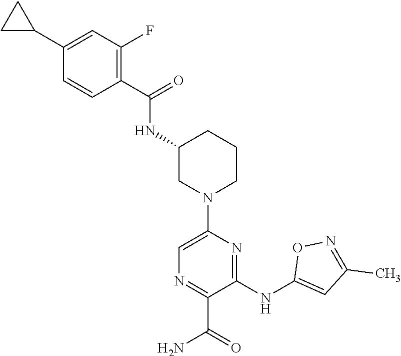 [SMILES: Cc1cc(Nc2nc(N3CCC[C@@H](NC(=O)c4ccc(C5CC5)cc4F)C3)cnc2C(N)=O)on1]

In a similar manner as described in Example 40, 5-[(3R)-3-(4-cyclopropyl-2-fluorobenzamido)piperidin-1-yl]-3-[(3-methyl-1,2-oxazol-5-yl)amino]pyrazine-2 carboxamide (D-2) was prepared. MS found for C24H26FN7O3 as (M+H)⁺ 480.5.

¹H NMR (400 MHz, DMSO) δ 12.28 (s, 1H), 8.21 (d, J=5.52 Hz, 1H), 7.93 (s, 1H), 7.87 (s, 1H), 7.59 (br. s., 1H), 7.44 (t, J=7.91 Hz, 1H), 7.03-6.92 (m, 2H), 6.23 (s, 1H), 4.46 (d, J=10.29 Hz, 1H), 4.15-4.02 (m, 1H), 4.02-3.88 (m, 1H), 3.41-3.14 (m, 2H), 2.13 (s, 3H), 2.05-1.81 (m, 3H), 1.78-1.48 (m, 2H), 1.07-0.95 (m, 2H), 0.79-0.65 (m, 2H).

### Example D-3: Synthesis of 5-[(3R)-3-(4-cyclopropyl-2-fluorobenzamido)piperidin-1-yl]-3-[(3-phenyl-1,2-thiazol-5-yl)amino]pyrazine-2-carboxamide (D-3)

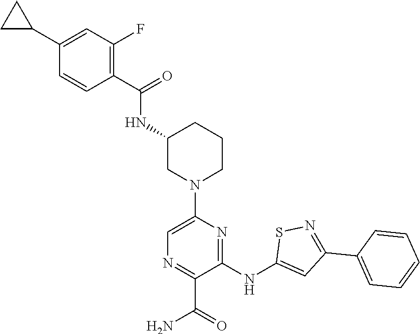 [SMILES: NC(=O)c1ncc(N2CCC[C@@H](NC(=O)c3ccc(C4CC4)cc3F)C2)nc1Nc1cc(-c2ccccc2)ns1]

In a similar manner as described in Example 40, 5-[(3R)-3-(4-cyclopropyl-2-fluorobenzamido)piperidin-1-yl]-3-[(3-phenyl-1,2-thiazol-5-yl)amino]pyrazine-2-carboxamide (D-3) was prepared. MS found for C29H28FN7O2S as (M+H)⁺ 558.3.

¹H NMR (500 MHz, DMSO) δ 12.46 (s, 1H), 8.27 (d, J=7.24 Hz, 1H), 7.99 (d, J=7.04 Hz, 2H), 7.94 (br. s., 1H), 7.85 (s, 1H), 7.67 (s, 1H), 7.58 (br. s., 1H), 7.02-6.90 (m, 2H), 4.42-4.15 (m, 2H), 4.07-3.91 (m, 1H), 3.65-3.45 (m, 2H), 2.09-1.87 (m, 2H), 1.82-1.47 (m, 3H), 1.07-0.94 (m, 2H), 0.81-0.64 (m, 2H).

### Example D-4: Synthesis of 5-[(3R)-3-(4-cyclopropyl-2-fluorobenzamido)piperidin-1-yl]-3-[(2-methyl-1,3-thiazol-5-yl)amino]pyrazine-2-carboxamide (D-4)

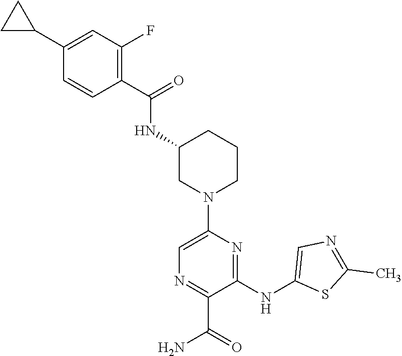 [SMILES: Cc1ncc(Nc2nc(N3CCC[C@@H](NC(=O)c4ccc(C5CC5)cc4F)C3)cnc2C(N)=O)s1]

In a similar manner as described in Example 40, 5-[(3R)-3-(4-cyclopropyl-2-fluorobenzamido)piperidin-1-yl]-3-[(3-phenyl-1,2-thiazol-5-yl)amino]pyrazine-2-carboxamide (D-4) was prepared. MS found for C24H26FN7O2S as (M+H)⁺ 496.2.

¹H NMR (500 MHz, DMSO) δ 11.81 (s, 1H), 8.22 (d, J=6.02 Hz, 1H), 7.80 (br. s., 1H), 7.73 (s, 1H), 7.47-7.40 (m, 2H), 7.37 (s, 1H), 7.01-6.92 (m, 2H), 4.53-4.07 (m, 2H), 4.06-3.93 (m, 1H), 3.50-2.97 (m, 2H), 2.45 (s, 3H), 2.03-1.93 (m, 1H), 2.11-1.51 (m, 4H), 1.04-0.97 (m, 2H), 0.78-0.70 (m, 2H).

### Example D-5: Synthesis of 3-{[4-(4-cyclopentylpiperazin-1-yl)phenyl]amino}-5-[(2R,3R)-3-(4-cyclopropyl-2-fluorobenzamido)-2-methylpiperidin-1-yl]pyrazine-2-carboxamide (D-5)

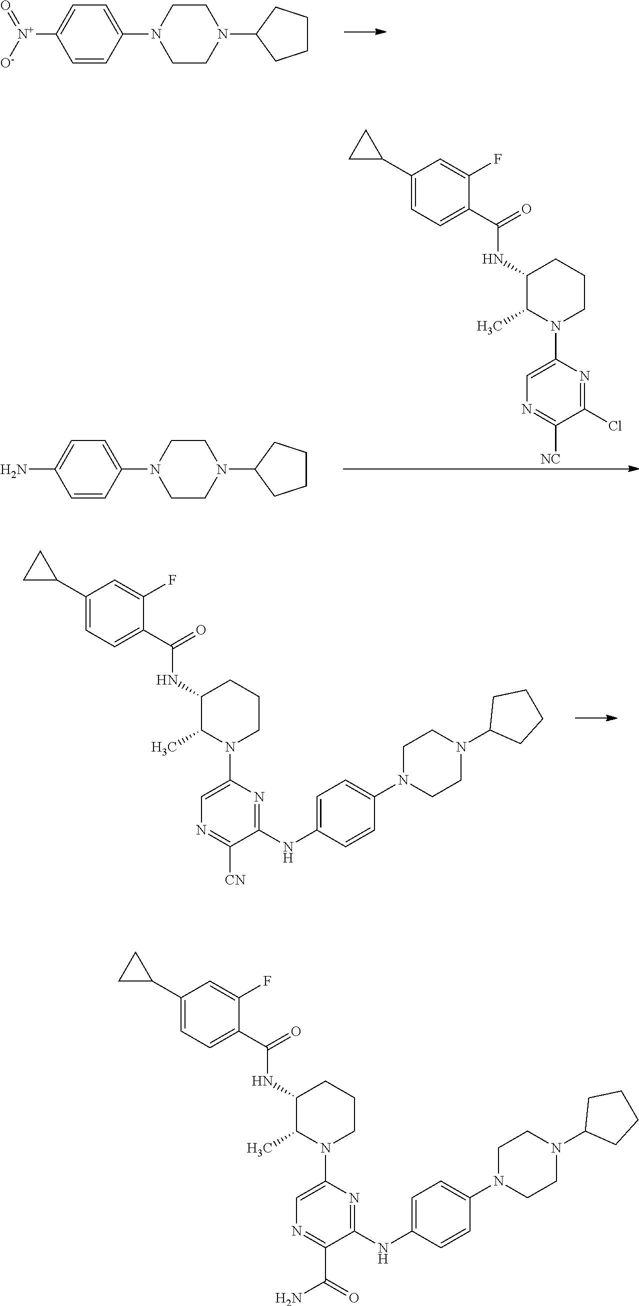 [SMILES: O=[N+]([O-])c1ccc(N2CCN(C3CCCC3)CC2)cc1 ; Nc1ccc(N2CCN(C3CCCC3)CC2)cc1 ; C[C@@H]1[C@H](NC(=O)c2ccc(C3CC3)cc2F)CCCN1c1cnc(C#N)c(Cl)n1 ; C[C@@H]1[C@H](NC(=O)c2ccc(C3CC3)cc2F)CCCN1c1cnc(C#N)c(Nc2ccc(N3CCN(C4CCCC4)CC3)cc2)n1 ; C[C@@H]1[C@H](NC(=O)c2ccc(C3CC3)cc2F)CCCN1c1cnc(C(N)=O)c(Nc2ccc(N3CCN(C4CCCC4)CC3)cc2)n1]

To a solution of 1-cyclopentyl-4-(4-nitrophenyl)piperazine (crude, 1.99 g, 7.09 mmol) in 50 mL of EtOH was added palladium on carbon (0.755 g, 0.709 mmol, 10% wt.). The mixture was reacted for 24 hours under H₂ pressure (4 bar). The solid was filtered off and the solution was concentrated in high vacuum to give crude target amine that was further purified by SCX cartridge to achieve 1-cyclopentyl-4-(4-aminophenyl)piperazine (1.201 g) as red solid. MS found for C15H23N3 as (M+H)⁺ 246.3.

In a similar manner as described in Example 40, 3-{[4-(4-cyclopentylpiperazin-1-yl)phenyl]amino}-5-[(2R,3R)-3-(4-cyclopropyl-2-fluorobenzamido)-2-methylpiperidin-1-yl]pyrazine-2-carboxamide (D-5) was prepared. MS found for C36H45FN8O2 as (M+H)⁺ 641.4. ¹H NMR (500 MHz, DMSO) δ 10.91 (s, 1H), 8.30 (d, J=7.28 Hz, 1H), 7.69 (d, J=2.00 Hz, 1H), 7.59 (s, 1H), 7.49 (t, J=7.78 Hz, 1H), 7.43 (d, J=9.03 Hz, 2H), 7.26 (d, J=2.01 Hz, 1H), 7.07-6.93 (m, 2H), 6.79 (d, J=9.03 Hz, 2H), 5.11 (br. s., 1H), 4.20-3.96 (m, 2H), 3.04 (t, J=11.92 Hz, 1H), 2.89 (br. s., 4H), 2.48-2.39 (m, 5H), 2.06-1.97 (m, 1H), 1.92-1.72 (m, 4H), 1.71-1.43 (m, 6H), 1.42-1.28 (m, 2H), 1.10 (d, J=6.78 Hz, 3H), 1.07-1.00 (m, 2H), 0.73-0.80 (m, 2H).

### Example D-6: Synthesis of 5-[(2R,3R)-3-(6-cyclopropyl-1-oxo-1,2-dihydroisoquinolin-2-yl)-2-methylpiperidin-1-yl]-3-{[4-(4-methylpiperazin-1-yl)phenyl]amino}pyrazine-2-carboxamide (D-6)

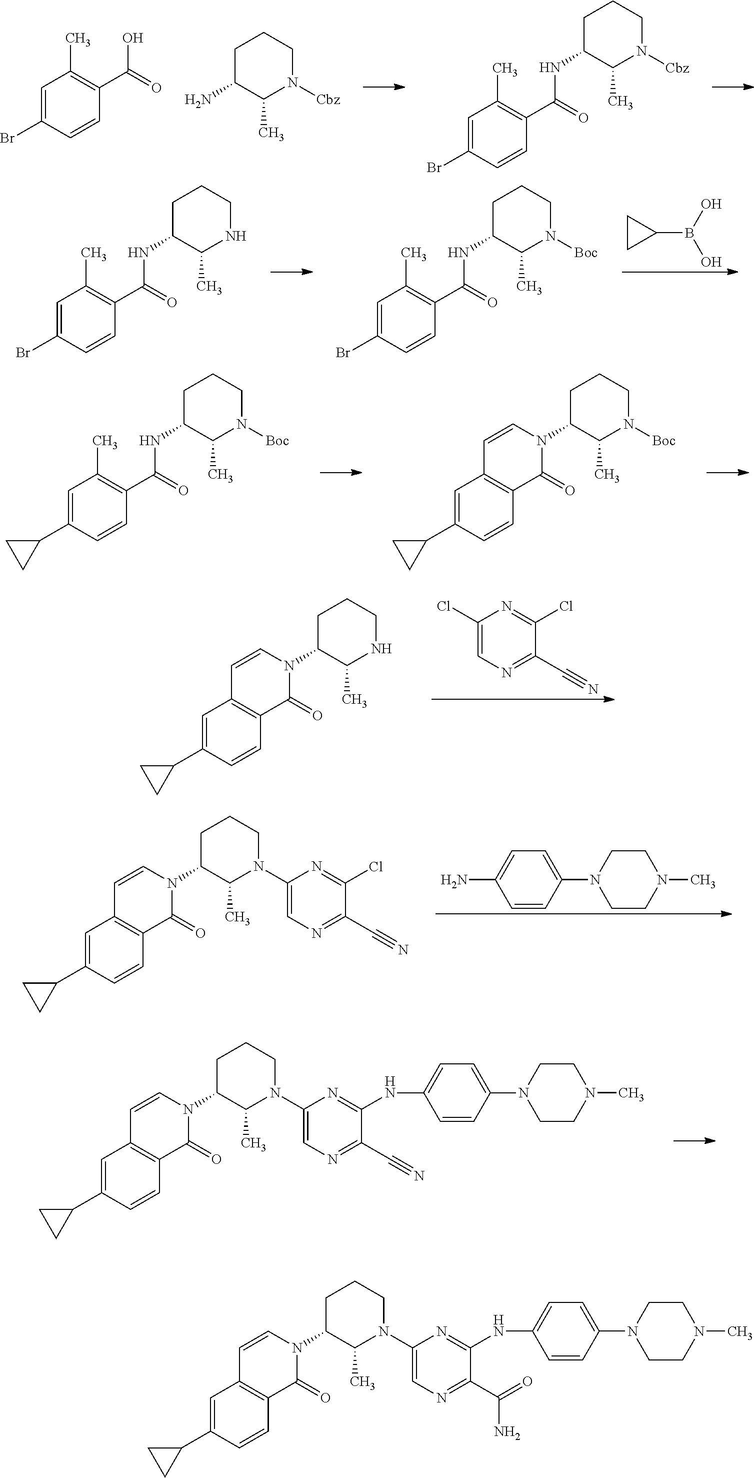

Benzyl (2R,3R)-3-amino-2-methylpiperidine-1-carboxylate (208 mg, 0.805 mmol) and 4-bromo-2-methylbenzoic acid (191 mg, 0.886 mmol) were dissolved in DMF (6 mL), then DIPEA (0.422 mL, 2.42 mmol) and PyBOP (628 mg, 1.208 mmol) were added and the mixture was stirred at room temperature 4 h. Water and DCM were added and the mixture was filtered through a phase separator. The organic solution was concentrated. The crude obtained was purified by silica flash chromatography with 0% to 45% of ethyl acetate in cyclohexane to afford benzyl (2R,3R)-3-(4-bromo-2-methylbenzamido)-2-methylpiperidine-1-carboxylate (305 mg, 85% yield) as a white solid. MS found for C22H25BrN2O3 as (M+H)⁺ 445.0.

To a solution of benzyl (2R,3R)-3-(4-bromo-2-methylbenzamido)-2-methylpiperidine-1-carboxylate (255 mg, 0.573 mmol) in DCM (6 mL) was added at room temperature TFA (1.5 mL) and trifluoromethanesulfonic acid (0.03 mL) and the resulting mixture was stirred at room temperature for 8 hours. The solvent was evaporated and the acids were quenched with Na₂SO₄, methanol was added and the solid was filtered off. The solution was evaporated and the residue was purified by SCX column to give 4-bromo-2-methyl-N-[(2R,3R)-2-methylpiperidin-3-yl]benzamide (1.02 g, quantitative). MS found for C14H19BrN2O as (M+H)⁺ 311.0.

To a solution of 4-bromo-2-methyl-N-[(2R,3R)-2-methylpiperidin-3-yl]benzamide (1.02 g, 0.68 mmol) in DCM (6 mL) was added Boc₂O (171 mg, 0.78 mmol) and TEA (0.57 mL, 4.08 mmol) and the mixture was stirred at room temperature for 6 hours. Water and DCM were added and the mixture was filtered through a phase separator. The organic phase was evaporated and the residue was purified by silica flash chromatography with 0% to 35% of ethyl acetate in cyclohexane to give tert-butyl (2R,3R)-3-(4-bromo-2-methylbenzamido)-2-methylpiperidine-1-carboxylate (270 mg, 97% yield) as a white solid. MS found for C19H27BrN2O3 as (M+H)⁺ 411.0.

To a solution of tert-butyl (2R,3R)-3-(4-bromo-2-methylbenzamido)-2-methylpiperidine-1-carboxylate (270 mg, 0.656 mmol) in 9 mL of toluene was added cyclopropylboronic acid (163 mg, 1.90 mmol), water (0.3 mL), K₃PO₄ (558 mg, 2.63 mmol) and Pd(PPh₃)₄ (151 mg, 0.131 mmol). The resulting mixture was degassed 10 minutes with a stream of N₂ then was stirred at 105° C. for 3 hours. Water was added and the product was extracted with ethyl acetate (three times). The collected organic phases were dried over Na₂SO₄, filtered and concentrated. The residue obtained was purified by silica flash chromatography with 0% to 35% of ethyl acetate in cyclohexane to give tert-butyl (2R,3R)-3-(4-cyclopropyl-2-methylbenzamido)-2-methylpiperidine-1-carboxylate (211 mg, 86% yield) as a white solid. MS found for C22H32N2O3 as (M+H)⁺ 373.1.

To a solution of tert-butyl (2R,3R)-3-(4-cyclopropyl-2-methylbenzamido)-2-methylpiperidine-1-carboxylate (97 mg, 0.260 mmol) in 5 mL of dry THF, cooled at −15° C., was added at the same temperature (internal temperature) 0.35 mL of BuLi (2.5 M in hexane) dropwise until the solution became deep red. After 10 minutes of stirring at −15° C. (internal temperature) was added dry DMF (0.30 mL) dropwise and the mixture was stirred for 15 minutes at the same temperature. Then 1 mL of aqueous HCl 1M was added dropwise (internal temperature reached 0° C.) and the mixture was stirred for 15 minutes. Water was added and the product was extracted with ethyl acetate (three times). The collected organic layers were dried over Na₂SO₄, filtered, concentrated and the residue was purified by silica flash chromatography with 0% to 35% of ethyl acetate in cyclohexane to give tert-butyl (2R,3R)-3-(6-cyclopropyl-1-oxo-1,2-dihydroisoquinolin-2-yl)-2-methylpiperidine-1-carboxylate (45 mg, 45% yield) as a white solid. MS found for C23H30N2O3 as (M+H)⁺ 383.2.

To a solution of tert-butyl (2R,3R)-3-(6-cyclopropyl-1-oxo-1,2-dihydroisoquinolin-2-yl)-2-methylpiperidine-1-carboxylate (45 mg. 0.0118 mmol) in 3 mL of DCM was added at room temperature TFA (1 mL). The solution was stirred at room temperature for 2 hours. The solvent was evaporated. The residue was diluted in DCM, K₂CO₃ was added and the solid was filtered off. The organic solution was evaporated to give 6-cyclopropyl-2-[(2R,3R)-2-methylpiperidin-3-yl]-1,2-dihydroisoquinolin-1-one (36 mg, quant. yield) as a colourless oil. MS found for C18H22N2O as (M+H)⁺ 283.1.

3,5-Dichloropyrazine-2-carbonitrile (24 mg, 0.138 mmol), 6-cyclopropyl-2-[(2R,3R)-2-methylpiperidin-3-yl]-1,2-dihydroisoquinolin-1-one (36 mg, 0.118 mmol) and DIEA (0.050 mL, 0.287 mmol) were dissolved in EtOH (2.5 mL) and stirred at 40° C. for 100 minutes. The reaction was quenched with aqueous NH₄Cl. DCM was added and the mixture was filtered through a phase separator. The organic layer was concentrated under high vacuum and the residue was purified by silica flash chromatography with 0% to 40% of ethyl acetate in cyclohexane to give 3-chloro-5-[(2R,3R)-3-(6-cyclopropyl-1-oxo-1,2-dihydroisoquinolin-2-yl)-2-methylpiperidin-1-yl]pyrazine-2-carbonitrile (39 mg, 79% yield) as a white solid. MS found for C23H22ClN5O as (M+H)⁺ 420.1.

In a similar manner as described in Example 40, 5-[(2R,3R)-3-(6-cyclopropyl-1-oxo-1,2-dihydroisoquinolin-2-yl)-2-methylpiperidin-1-yl]-3-{[4-(4-methylpiperazin-1-yl)phenyl]amino}pyrazine-2-carboxamide (D-6) was prepared from 3-chloro-5-[(2R,3R)-3-(6-cyclopropyl-1-oxo-1,2-dihydroisoquinolin-2-yl)-2-methylpiperidin-1-yl]pyrazine-2-carbonitrile. MS found for C34H40N8O2 as (M+H)⁺ 593.2.

¹H NMR (500 MHz, DMSO) δ 10.93 (br. s., 1H), 8.26 (d, J=8.33 Hz, 1H), 7.78-7.69 (m, 1H), 7.66-7.60 (m, 1H), 7.48-7.42 (m, 3H), 7.41-7.38 (m, 1H), 7.33-7.27 (m, 1H), 7.27-7.22 (m, 1H), 6.73 (d, J=6.14 Hz, 2H), 6.60 (d, J=7.45 Hz, 1H), 5.57-5.14 (m, 1H), 4.93 (d, J=13.37 Hz, 1H), 4.37-3.95 (m, 1H), 3.09 (t, J=12.39 Hz, 1H), 2.95-2.73 (m, 4H), 2.45-2.29 (m, 5H), 2.21 (s, 3H), 2.15-2.06 (m, 1H), 2.04-1.70 (m, 3H), 1.13-1.02 (m, 2H), 0.95-0.77 (m, 5H).

### Example D-7: Synthesis of 5-[(2R,3R)-3-(4-cyclopropyl-2-fluorobenzamido)-2-methylpiperidin-1-yl]-3-({4-[4-(propan-2-yl)piperazin-1-yl]phenyl}amino)pyrazine-2-carboxamide (D-7)

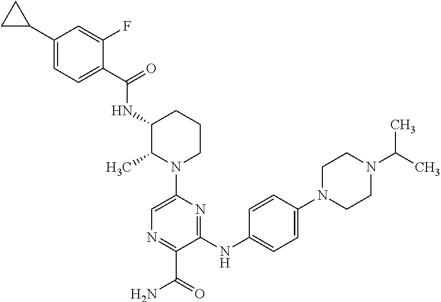 [SMILES: CC(C)N1CCN(c2ccc(Nc3nc(N4CCC[C@@H](NC(=O)c5ccc(C6CC6)cc5F)[C@H]4C)cnc3C(N)=O)cc2)CC1]

In a similar manner as described in Example 40, 5-[(2R,3R)-3-(4-cyclopropyl-2-fluorobenzamido)-2-methylpiperidin-1-yl]-3-({4-[4-(propan-2-yl)piperazin-1-yl]phenyl}amino)pyrazine-2-carboxamide (D-7) was prepared. MS found for C34H43FN8O2 as (M+H)⁺ 615.3.

¹H NMR (500 MHz, DMSO) δ 10.92 (s, 1H), 8.31 (d, J=7.55 Hz, 1H), 7.70 (d, J=1.90 Hz, 1H), 7.59 (s, 1H), 7.49 (t, J=7.75 Hz, 1H), 7.44 (d, J=8.92 Hz, 2H), 7.27 (d, J=1.92 Hz, 1H), 7.06-6.97 (m, 2H), 6.79 (d, J=8.90 Hz, 2H), 5.12 (br. s., 1H), 4.21-3.95 (m, 2H), 3.04 (t, J=12.21 Hz, 1H), 2.89 (br. s., 4H), 2.64 (br. s., 1H), 2.56-2.38 (m, 4H), 2.06-1.97 (m, 1H), 1.90-1.77 (m, 2H), 1.70-1.53 (m, 2H), 1.10 (d, J=6.86 Hz, 3H), 1.06-0.96 (m, 8H), 0.81-0.71 (m, 2H).

### Example D-8: Synthesis of 5-[(2R,3R)-3-(4-cyclopropyl-2-fluorobenzamido)-2-methylpiperidin-1-yl]-3-{[5-(4-methylpiperazin-1-yl)pyridin-2-yl]amino}pyrazine-2-carboxamide (D-8)

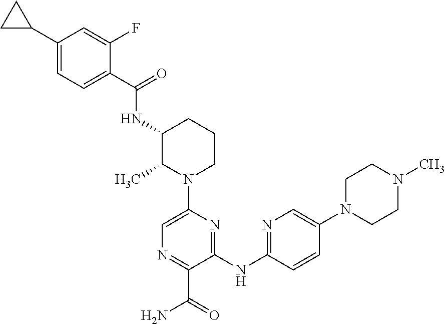 [SMILES: C[C@@H]1[C@H](NC(=O)c2ccc(C3CC3)cc2F)CCCN1c1cnc(C(N)=O)c(Nc2ccc(N3CCN(C)CC3)cn2)n1]

In a similar manner as described in Example 40, 5-[(2R,3R)-3-(4-cyclopropyl-2-fluorobenzamido)-2-methylpiperidin-1-yl]-3-{[5-(4-methylpiperazin-1-yl)pyridin-2-yl]amino}pyrazine-2-carboxamide (D-8) was prepared. MS found for C31H38FN9O2 as (M+H)⁺ 588.3.

¹H NMR (500 MHz, DMSO) δ 11.37 (br. s, 1H), 8.35 (d, J=7.14 Hz, 1H), 8.12 (d, J=9.06 Hz, 1H), 7.96 (d, J=3.02 Hz, 1H), 7.76 (d, J=2.20 Hz, 1H), 7.68 (s, 1H), 7.49 (t, J=7.96 Hz, 1H), 7.36 (d, J=2.47 Hz, 1H), 7.25 (d, J=7.14 Hz, 1H), 7.07-6.94 (m, 2H), 5.34-5.05 (m, 1H), 4.22-3.92 (m, 2H), 3.14-3.05 (m, 1H), 2.99 (br. s., 4H), 2.45-2.35 (m, 4H), 2.21 (s, 3H), 2.09-1.94 (m, 1H), 1.92-1.53 (m, 4H), 1.11 (d, J=6.86 Hz, 3H), 1.04 (dd, J=8.23, 2.20 Hz, 2H), 0.76 (dd, J=4.94, 1.92 Hz, 2H).

### Example D-9: Synthesis of 3-{[4-(1-cyclopentyl-4-methylpiperidin-4-yl)phenyl]amino}-5-[(2R,3R)-2-methyl-3-{[(pyridin-3-yl)carbamoyl]amino}piperidin-1-yl]pyrazine-2-carboxamide (D-9)

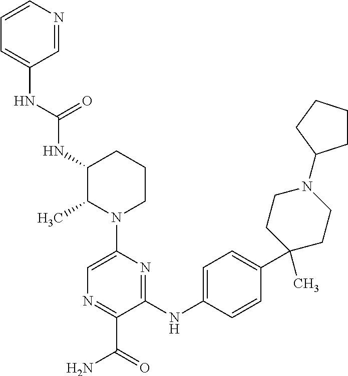 [SMILES: C[C@@H]1[C@H](NC(=O)Nc2cccnc2)CCCN1c1cnc(C(N)=O)c(Nc2ccc(C3(C)CCN(C4CCCC4)CC3)cc2)n1]

In a similar manner as described in Example 1, 3-{[4-(1-cyclopentyl-4-methylpiperidin-4-yl)phenyl]amino}-5-[(2R,3R)-2-methyl-3-{[(pyridin-3-yl)carbamoyl]amino}piperidin-1-yl]pyrazine-2-carboxamide (D-9) was prepared. MS found for C34H45N9O2 as (M+H)⁺ 612.6.

¹H NMR (400 MHz, DMSO) δ 11.25 (s, 1H), 8.68 (s, 1H), 8.57 (d, J=2.52 Hz, 1H), 8.13 (dd, J=4.66, 1.37 Hz, 1H), 8.03-7.95 (m, 1H), 7.64 (s, 1H), 7.55 (d, J=8.66 Hz, 2H), 7.33 (d, J=1.75 Hz, 1H), 7.26 (dd, J=8.28, 4.66 Hz, 1H), 7.20 (d, J=8.66 Hz, 2H), 6.55 (d, J=7.67 Hz, 1H), 5.12 (br. s., 1H), 4.12 (d, J=11.07 Hz, 1H), 3.86-3.74 (m, 1H), 3.13-2.99 (m, 1H), 2.41-2.20 (m, 5H), 1.89-1.14 (m, 16H), 1.10 (d, J=6.91 Hz, 3H), 1.02 (s, 3H).

### Example D-10: Synthesis of 5-[(2R,3R)-3-(4-cyclopropyl-2-fluorobenzamido)-2-methylpiperidin-1-yl]-3-{[4-(4-methyl-2-oxopiperazin-1-yl)phenyl]amino}pyrazine-2-carboxamide (D-10)

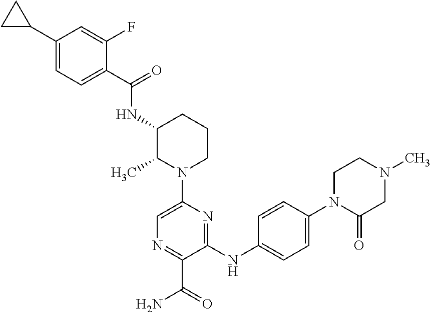 [SMILES: C[C@@H]1[C@H](NC(=O)c2ccc(C3CC3)cc2F)CCCN1c1cnc(C(N)=O)c(Nc2ccc(N3CCN(C)CC3=O)cc2)n1]

In a similar manner as described in Example 40, 5-[(2R,3R)-3-(4-cyclopropyl-2-fluorobenzamido)-2-methylpiperidin-1-yl]-3-{[4-(4-methyl-2-oxopiperazin-1-yl)phenyl]amino}pyrazine-2-carboxamide (D-10) was prepared. MS found for C32H37FN8O3 as (M+H)⁺ 601.2.

¹H NMR (500 MHz, DMSO) δ 11.32 (s, 1H), 8.30 (d, J=7.27 Hz, 1H), 7.79 (br. s., 1H), 7.68 (s, 1H), 7.62 (d, J=8.78 Hz, 2H), 7.46 (t, J=7.89 Hz, 1H), 7.38 (br. s., 1H), 7.20 (d, J=8.78 Hz, 2H), 7.04-6.96 (m, 2H), 5.12 (br. s., 1H), 4.17 (br. s., 1H), 4.07-3.97 (m, 1H), 3.52 (t, J=5.15 Hz, 2H), 3.12-3.02 (m, 3H), 2.64 (t, J=5.49 Hz, 2H), 2.27 (s, 3H), 2.07-1.96 (m, 1H), 1.92-1.77 (m, 2H), 1.74-1.53 (m, 2H), 1.11 (d, J=6.86 Hz, 3H), 1.07-1.00 (m, 2H), 0.81-0.72 (m, 2H).

### Example D-11: Synthesis of 5-[(2R,3R)-2-methyl-3-[4-(trifluoromethyl)benzamido]piperidin-1-yl]-3-{[4-(4-methylpiperazin-1-yl)phenyl]amino}pyrazine-2-carboxamide (D-11)

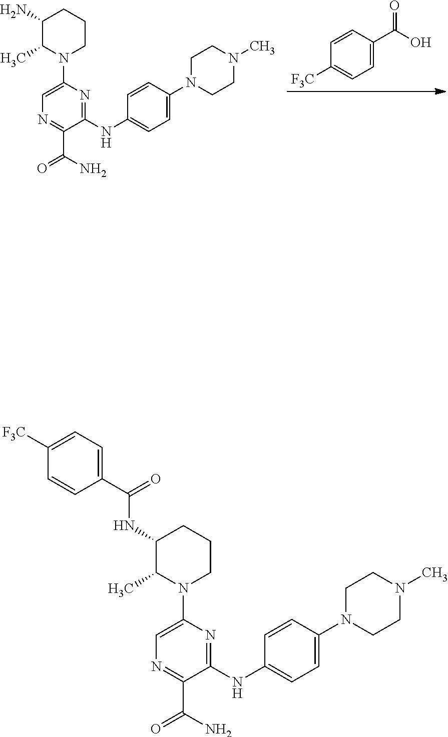

5-[(2R,3R)-3-amino-2-methylpiperidin-1-yl]-3-{[4-(4-methylpiperazin-1-yl)phenyl]amino}pyrazine-2-carboxamide (crude, 201 mg, 0.163 mmol) and 4-(trifluoromethyl)benzoic acid (39 mg, 0.204 mmol) were dissolved in DMF (3.5 mL), then DIPEA (0.284 mL, 1.63 mmol) and TBTU (79 mg, 0.245 mmol) were added and the mixture was stirred at room temperature 2 h. Water was added and the mixture was extracted with ethyl acetate (three times). The collected organic layers were dried over Na₂SO₄, filtered and concentrated. The crude obtained was purified by preparative HPLC to afford 5-[(2R,3R)-2-methyl-3-[4-(trifluoromethyl)benzamido]piperidin-1-yl]-3-{[4-(4-methylpiperazin-1-yl)phenyl]amino}pyrazine-2-carboxamide (27.3 mg, 28% yield) as a yellow solid. MS found for C30H35F3N8O2 as (M+H)⁺ 597.3.

¹H NMR (500 MHz, DMSO) δ 10.93 (br. s., 1H), 8.72 (d, J=7.55 Hz, 1H), 8.13 (d, J=8.10 Hz, 2H), 7.89 (d, J=8.23 Hz, 2H), 7.71 (br. s., 1H), 7.60 (s, 1H), 7.43 (d, J=9.06 Hz, 2H), 7.28 (br. s., 1H), 6.77 (d, J=8.23 Hz, 2H), 5.14 (br. s., 1H), 4.25-4.05 (m, 2H), 3.07 (t, J=12.62 Hz, 1H), 2.88 (br. s., 4H), 2.39-2.27 (m, 4H), 2.17 (s, 3H), 1.95 (qd, J=12.92, 3.77 Hz, 1H), 1.86 (d, J=12.76 Hz, 1H), 1.76-1.54 (m, 2H), 1.09 (d, J=6.72 Hz, 3H).

### Example D-12: Synthesis of 5-[(2R,3R)-3-(4-cyclopropyl-2-fluorobenzamido)-2-methylpiperidin-1-yl]-3-({4-[(2S)-2,4-dimethylpiperazin-1-yl]phenyl}amino)pyrazine-2-carboxamide (D-12)

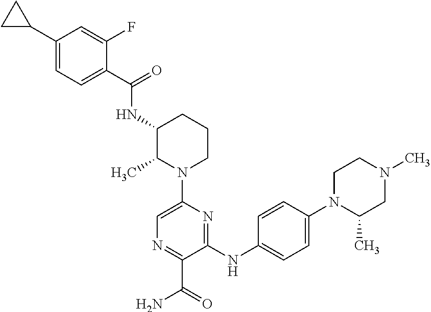 [SMILES: C[C@@H]1[C@H](NC(=O)c2ccc(C3CC3)cc2F)CCCN1c1cnc(C(N)=O)c(Nc2ccc(N3CCN(C)C[C@@H]3C)cc2)n1]

In a similar manner as described in Example 40, 5-[(2R,3R)-3-(4-cyclopropyl-2-fluorobenzamido)-2-methylpiperidin-1-yl]-3-({4-[(2S)-2,4-dimethylpiperazin-1-yl]phenyl}amino)pyrazine-2-carboxamide (D-12) was prepared. MS found for C33H41FN8O2 as (M+H)⁺ 601.7.

¹H NMR (500 MHz, DMSO) δ 10.92 (s, 1H), 8.28 (d, J=7.34 Hz, 1H), 7.70 (br. s., 1H), 7.58 (s, 1H), 7.53-7.46 (m, 1H), 7.43 (d, J=8.80 Hz, 2H), 7.27 (br. s., 1H), 7.05-6.94- (m, 2H), 6.76 (d, J=8.80 Hz, 2H), 5.28-4.97 (m, 1H), 4.21-3.94 (m, 2H), 3.59 (br. s., 1H), 3.03 (t, J=12.47 Hz, 1H), 2.98-2.87 (m, 1H), 2.85-2.72 (m, 1H), 2.59 (d, J=10.76 Hz, 1H), 2.42 (d, J=9.78 Hz, 1H), 2.20 (dd, J=10.76, 2.93 Hz, 1H), 2.16 (s, 3H), 2.06-1.97 (m, 2H), 1.87-1.77 (m, 2H), 1.71-1.51 (m, 2H), 1.07 (d, J=6.85 Hz, 3H), 1.05-1.00 (m, 2H), 0.85 (d, J=6.36 Hz, 3H), 0.78-0.73 (m, 2H).

### Example D-13: Synthesis of 5-[(2R,3R)-3-(4-cyclopropyl-2-fluorobenzamido)-2-methylpiperidin-1-yl]-3-{[6-(4-methylpiperazin-1-yl)pyridin-3-yl]amino}pyrazine-2-carboxamide (D-13)

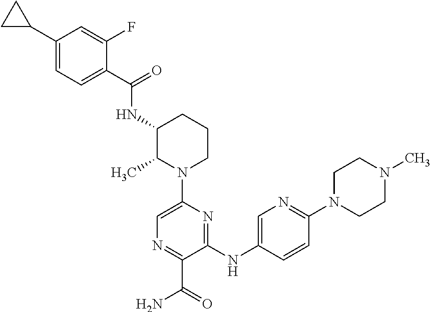 [SMILES: C[C@@H]1[C@H](NC(=O)c2ccc(C3CC3)cc2F)CCCN1c1cnc(C(N)=O)c(Nc2ccc(N3CCN(C)CC3)nc2)n1]

In a similar manner as described in Example 40, 5-[(2R,3R)-3-(4-cyclopropyl-2-fluorobenzamido)-2-methylpiperidin-1-yl]-3-{[6-(4-methylpiperazin-1-yl)pyridin-3-yl]amino}pyrazine-2-carboxamide (D-13) was prepared. MS found for C31H38FN9O2 as (M+H)⁺ 588.7.

¹H NMR (500 MHz, DMSO) δ 10.83 (s, 1H), 8.29 (d, J=7.56 Hz, 1H), 8.16 (br. s., 1H), 7.94 (d, J=8.00 Hz, 1H), 7.71 (br. s., 1H), 7.61 (s, 1H), 7.48 (t, J=7.95 Hz, 1H), 7.30 (br. s., 1H), 7.00 (dd, J=7.34, 6.03 Hz, 2H), 6.70 (d, J=9.10 Hz, 1H), 5.00 (br. s., 1H), 4.23-3.95 (m, 2H), 3.27 (br. s., 4H), 3.02 (t, J=11.84 Hz, 1H), 2.31 (t, J=4.77 Hz, 4H), 2.19 (s, 3H), 2.07-1.95 (m, 1H), 1.92-1.73 (m, 2H), 1.73-1.48 (m, 2H), 1.13-0.99 (m, 5H), 0.80-0.73 (m, 2H).

### Example D-14: Synthesis of 5-[(2R,3R)-3-(4-tert-butylbenzamido)-2-methylpiperidin-1-yl]-3-{[4-(4-methylpiperazin-1-yl)phenyl]amino}pyrazine-2-carboxamide (D-14)

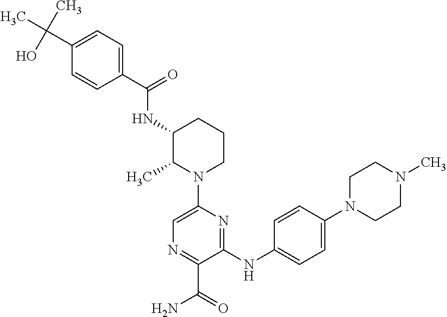 [SMILES: C[C@@H]1[C@H](NC(=O)c2ccc(C(C)(C)O)cc2)CCCN1c1cnc(C(N)=O)c(Nc2ccc(N3CCN(C)CC3)cc2)n1]

In a similar manner as described in Example D-11, 5-[(2R,3R)-3-(4-tert-butylbenzamido)-2-methylpiperidin-1-yl]-3-{[4-(4-methylpiperazin-1-yl)phenyl]amino}pyrazine-2-carboxamide (D-14) was prepared. MS found for C33H44N8O2 as (M+H)⁺ 585.4.

¹H NMR (500 MHz, DMSO) δ 10.93 (s, 1H), 8.36 (d, J=7.45 Hz, 1H), 7.89 (d, J=8.44 Hz, 2H), 7.70 (br. s., 1H), 7.59 (s, 1H), 7.50 (d, J=8.55 Hz, 2H), 7.27 (br. s., 1H), 6.81 (d, J=8.77 Hz, 2H), 5.15 (br. s., 1H), 4.26-3.98 (m, 2H), 3.06 (t, J=12.11 Hz, 1H), 3.00-2.85 (m, 4H). 2.44-2.29 (m, 4H), 2.06-1.78 (m, 2H), 1.75-1.51 (m, 2H), 1.32 (s, 9H), 1.06 (d, J=6.80 Hz, 3H).

### Example D-15: Synthesis of N-[(3R)-1-{5-carbamoyl-6-[(3-methyl-1,2-thiazol-5-yl)amino]pyrazin-2-yl}piperidin-3-yl]-1-oxo-2,3-dihydro-1H-isoindole-2-carboxamide (D-15)

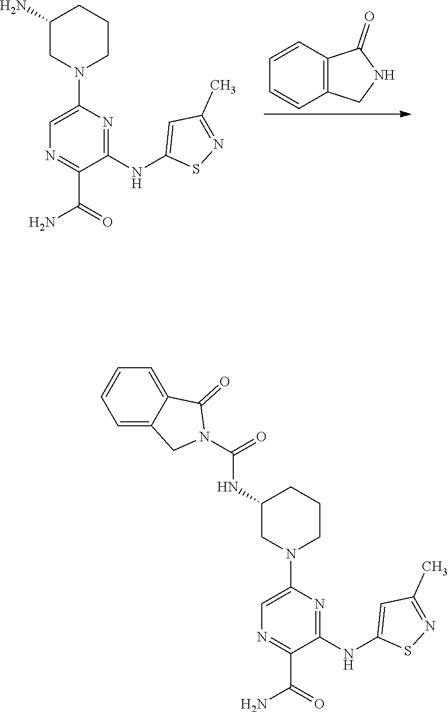 [SMILES: Cc1cc(Nc2nc(N3CCC[C@@H](N)C3)cnc2C(N)=O)sn1 ; O=C1NCc2ccccc21 ; Cc1cc(Nc2nc(N3CCC[C@@H](NC(=O)N4Cc5ccccc5C4=O)C3)cnc2C(N)=O)sn1]

To a solution of isoindolin-1-one (40 mg, 0.304 mmol) in 3 mL of dry DCM was added at room temperature triphosgene (105 mg, 0.354 mmol) and TEA (0.107 mL, 0.828 mmol) dropwise. The mixture was stirred at room temperature for 30 minutes, then a solution of 5-[(3R)-3-aminopiperidin-1-yl]-3-[(3-methyl-1,2-thiazol-5-yl)amino]pyrazine-2-carboxamide (92 mg, 0.276 mmol) in 3 mL of dry DCM was added dropwise to the reaction mixture at room temperature. The reaction was stirred overnight at room temperature. Water was added and the mixture was extracted with ethyl acetate (three times). The collected organic layers were dried over Na₂SO₄, filtered and concentrated. The crude obtained was purified by preparative HPLC to afford N-[(3R)-1-{5-carbamoyl-6-[(3-methyl-1,2-thiazol-5-yl)amino]pyrazin-2-yl}piperidin-3-yl]-1-oxo-2,3-dihydro-1H-isoindole-2-carboxamide (D-15) (25.2 mg, 19% yield) as a red solid. MS found for C23H24N8O3S as (M+H)⁺ 493.2.

¹H NMR (500 MHz, DMSO) δ 12.23 (s, 1H), 8.69 (d, J=7.67 Hz, 1H), 7.95-7.81 (m, 2H), 7.77-7.70 (m, 2H), 7.69-7.64 (m, 1H), 7.58-7.48 (m, 2H), 6.78 (s, 1H), 4.92-4.61 (m, 2H), 4.21-3.74 (m, 5H), 2.26 (s, 3H), 2.11-1.96 (m, 1H), 1.93-1.62 (m, 3H).

### Example D-16: Synthesis of 5-[(2R,3R)-3-(2-chloro-4-cyclopropylbenzamido)-2-methylpiperidin-1-yl]-3-{[4-(4-methylpiperazin-1-yl)phenyl]amino}pyrazine-2-carboxamide (D-16)

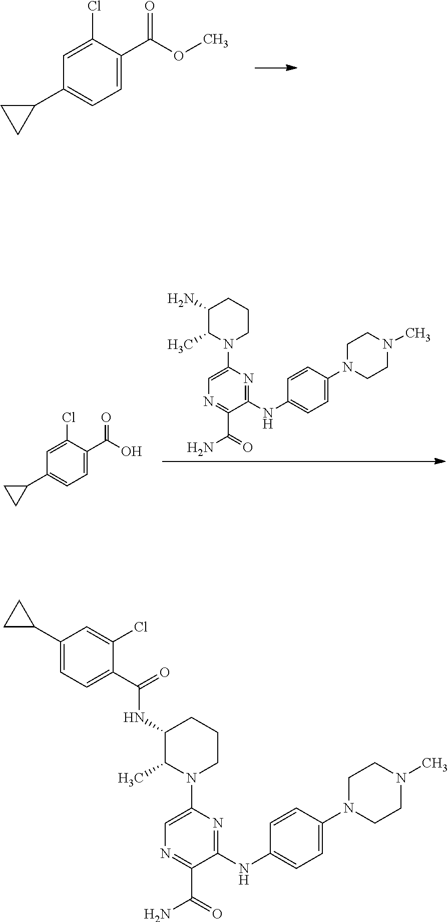

To a solution of methyl 2-chloro-4-cyclopropylbenzoate (crude, 751 mg, 1.98 mmol) in 2.5 mL of THF and 2.5 mL of MeOH was added at room temperature a solution of LiOH (96 mg, 4.01 mmol) in 2.5 mL of water. The reaction mixture was stirred at room temperature overnight. The reaction mixture was diluted with ethyl acetate and water. The basic aqueous solution was acidified with 1.5 mL of aqueous HCl 6N. The product was extracted with ethyl acetate (twice) and the collected organic layers were dried over Na₂SO₄, filtered and evaporated in high vacuum to yield 2-chloro-4-cyclopropylbenzoic acid (crude, 436 mg, quant. yield) as grey solid. MS found for C10H9ClO2 as (M+H)⁺ 197.0.

In a similar manner as described in Example D-11, 5-[(2R,3R)-3-(2-chloro-4-cyclopropylbenzamido)-2-methylpiperidin-1-yl]-3-{[4-(4-methylpiperazin-1-yl)phenyl]amino}pyrazine-2-carboxamide (D-16) was prepared. MS found for C32H39ClN8O2 as (M+H)⁺ 603.3.

¹H NMR (500 MHz, DMSO) δ 11.04 (s, 1H), 8.51 (d, J=7.69 Hz, 1H), 7.72 (br. s., 1H), 7.59 (s, 1H), 7.46 (d, J=9.33 Hz, 2H), 7.36-7.17 (m, 3H), 7.10 (dd, J=7.68, 1.65 Hz, 1H), 6.85 (d, J=8.78 Hz, 2H), 5.12 (br. s., 1H), 4.11 (br. s., 1H), 4.05-3.93 (m, 1H), 3.13-3.01 (m, 1H), 2.97 (br. s., 4H), 2.38 (t, J=4.94 Hz, 4H), 2.20 (s, 3H), 2.04-1.96 (m, 1H), 1.88-1.74 (m, 2H), 1.70-1.49 (m, 2H), 1.13 (d, J=6.59 Hz, 3H), 1.05-0.98 (m, 2H), 0.77-0.69 (m, 2H).

### Example D-17: Synthesis of 5-[(2R,3R)-3-(3-chloro-4-cyclopropylbenzamido)-2-methylpiperidin-1-yl]-3-{[4-(4-methylpiperazin-1-yl)phenyl]amino}pyrazine-2-carboxamide (D-17)

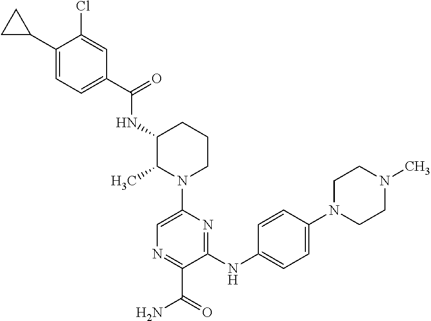 [SMILES: C[C@@H]1[C@H](NC(=O)c2ccc(C3CC3)c(Cl)c2)CCCN1c1cnc(C(N)=O)c(Nc2ccc(N3CCN(C)CC3)cc2)n1]

In a similar manner as described in Example D-11, 5-[(2R,3R)-3-(3-chloro-4-cyclopropylbenzamido)-2-methylpiperidin-1-yl]-3-{[4-(4-methylpiperazin-1-yl)phenyl]amino}pyrazine-2-carboxamide (D-17) was prepared. MS found for C32H39ClN8O2 as (M+H)⁺ 603.3.

¹H NMR (500 MHz, DMSO) δ 10.95 (s, 1H), 8.52 (d, J=7.69 Hz, 1H), 8.00 (d, J=1.65 Hz, 1H), 7.84 (d, J=8.23 Hz, 1H), 7.71 (br. s., 1H), 7.59 (s, 1H), 7.45 (d, J=8.78 Hz, 2H), 7.28 (br. s., 1H), 7.13 (d, J=8.23 Hz, 1H), 6.79 (d, J=8.78 Hz, 2H), 5.35-4.96 (m, 1H), 4.27-3.97 (m, 2H), 3.07 (t, J=12.35 Hz, 1H), 2.92 (br. s., 4H), 2.42-2.32 (m, 4H), 2.28-2.17 (m, 4H), 2.00-1.82 (m, 2H), 1.73-1.51 (m, 2H), 1.13-1.08 (m, 2H), 1.06 (d, J=7.14 Hz, 3H), 0.84-0.76 (m, 2H).

### Example D-18: Synthesis of 5-[(2R,3R)-3-(6-cyclopropylpyridine-3-amido)-2-methylpiperidin-1-yl]-3-[(1-methyl-1H-pyrazol-4-yl)amino]pyrazine-2-carboxamide (D-18)

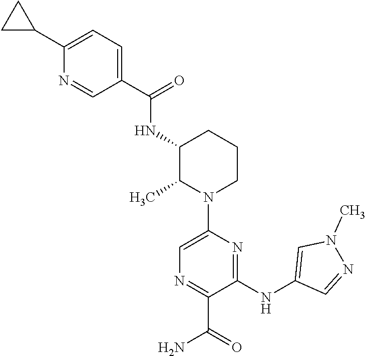 [SMILES: C[C@@H]1[C@H](NC(=O)c2ccc(C3CC3)nc2)CCCN1c1cnc(C(N)=O)c(Nc2cnn(C)c2)n1]

In a similar manner as described in Example D-11, 5-[(2R,3R)-3-(6-cyclopropylpyridine-3-amido)-2-methylpiperidin-1-yl]-3-[(1-methyl-1H-pyrazol-4-yl)amino]pyrazine-2-carboxamide (D-18) was prepared. MS found for C24H29N9O2 as (M+H)⁺ 476.2.

¹H NMR (500 MHz, DMSO) δ 10.87 (s, 1H), 8.89 (d, J=1.96 Hz, 1H), 8.50 (d, J=6.65 Hz, 1H), 8.11 (dd, J=8.22, 2.35 Hz, 1H), 8.01 (s, 1H), 7.69 (br. s., 1H), 7.57 (s, 1H), 7.47 (s, 1H), 7.41 (d, J=7.83 Hz, 1H), 7.27 (br. s., 1H), 5.47-5.12 (m, 1H), 4.22-3.95 (m, 2H), 3.76 (s, 3H), 3.15-3.00 (m, 1H), 2.23-2.12 (m, 1H), 2.02-1.78 (m, 2H), 1.77-1.50 (m, 2H), 1.10 (d, J=7.04 Hz, 3H), 0.94-1.03 (m, 4H).

### Example D-19: Synthesis of 3-[(1-methyl-1H-pyrazol-4-yl)amino]-5-[(2R,3R)-2-methyl-3-{1,5,5-trimethyl-1H,4H,5H,6H-cyclopenta[b]pyrrole-2-amido}piperidin-1-yl]pyrazine-2-carboxamide (D-19)

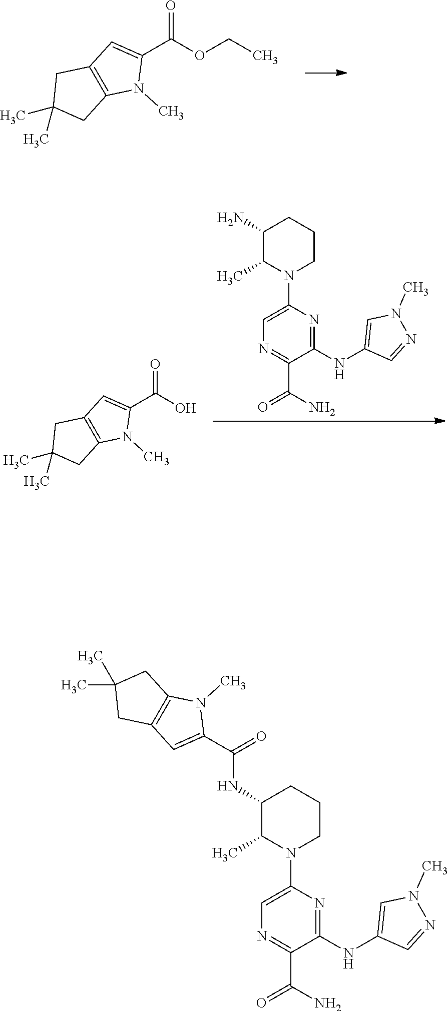 [SMILES: CCOC(=O)c1cc2c(n1C)CC(C)(C)C2 ; Cn1c(C(=O)O)cc2c1CC(C)(C)C2 ; C[C@@H]1[C@H](N)CCCN1c1cnc(C(N)=O)c(Nc2cnn(C)c2)n1 ; C[C@@H]1[C@H](NC(=O)c2cc3c(n2C)CC(C)(C)C3)CCCN1c1cnc(C(N)=O)c(Nc2cnn(C)c2)n1]

To a solution of ethyl 5,5-dimethyl-1H,4H,5H,6H-cyclopenta[b]pyrrole-2-carboxylate (505 mg, 2.44 mmol) in 10 mL of dry DMF was added at 0° C. NaH (116 mg, 2.49 mmol, 60% dispersion in mineral oil) and the resulting mixture was stirred at 0° C. for 5 minutes then was allowed to stir at room temperature for further 10 minutes. MeI (0.18 mL, 2.89 mmol) was added at room temperature and the mixture was stirred at room temperature for 1 hour. Then water was added dropwise and the product was extracted with ethyl acetate (three times). The collected organic layers were dried over Na₂SO₄ to give ethyl 1,5,5-trimethyl-1H,4H,5H,6H-cyclopenta[b]pyrrole-2-carboxylate (588 mg, quant. yield) as a white solid. MS found for C13H19NO2 as (M+H)⁺ 222.1.

To a solution of ethyl 1,5,5-trimethyl-1H,4H,5H,6H-cyclopenta[b]pyrrole-2-carboxylate (588 mg, 2.41 mmol) in 3 mL of THF and 3 mL of MeOH was added at room temperature a solution of LiOH.H₂O (202 mg, 4.82 mmol) in 3 mL of water. The reaction mixture was stirred at room temperature for 18 hours then at 65° C. for further 4 hours and then at 75° C. for further 6 hours. The reaction mixture was quenched with aqueous HCl (6N, 1.5 mL) and the product was extracted with ethyl acetate (three times). The collected organic layers were dried over Na₂SO₄, filtered and evaporated in high vacuum to give 1,5,5-trimethyl-1H,4H,5H,6H-cyclopenta[b]pyrrole-2-carboxylic acid (451 mg, 97% yield) as a grey solid. MS found for C11H15NO2 as (M+H)⁺ 194.2.

In a similar manner as described in Example D-11, 3-[(1-methyl-1H-pyrazol-4-yl)amino]-5-[(2R,3R)-2-methyl-3-{1,5,5-trimethyl-1H,4H,5H,6H-cyclopenta[b]pyrrole-2-amido}piperidin-1-yl]pyrazine-2-carboxamide (D-19) was prepared. MS found for C26H35N9O2 as (M+H)⁺ 506.3.

¹H NMR (500 MHz, DMSO) δ 10.83 (s, 1H), 7.90 (s, 1H), 7.74-7.63 (m, 2H), 7.58-7.51 (m, 2H), 7.27 (br. s., 1H), 6.67 (s, 1H), 5.10 (br. s., 1H), 4.13 (br. s., 1H), 4.00-3.89 (m, 1H), 3.77-3.64 (m, 6H), 3.12-2.98 (m, 1H), 2.52-2.48 (m, 2H), 2.37 (s, 2H), 1.96-1.79 (m, 2H), 1.71-1.48 (m, 2H), 1.18 (s, 6H), 1.08 (d, J=6.85 Hz, 3H).

### Example D-20: Synthesis of 5-[(2R,3R)-3-(2-chloro-4-cyclopropylbenzamido)-2-methylpiperidin-1-yl]-3-[(1-methyl-1H-pyrazol-4-yl)amino]pyrazine-2-carboxamide (D-20)

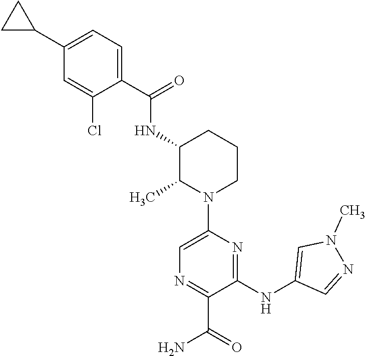 [SMILES: C[C@@H]1[C@H](NC(=O)c2ccc(C3CC3)cc2Cl)CCCN1c1cnc(C(N)=O)c(Nc2cnn(C)c2)n1]

In a similar manner as described in Example D-11, 5-[(2R,3R)-3-(2-chloro-4-cyclopropylbenzamido)-2-methylpiperidin-1-yl]-3-[(1-methyl-1H-pyrazol-4-yl)amino]pyrazine-2-carboxamide (D-20) was prepared. MS found for C25H29ClN8O2 as (M+H)⁺ 509.2.

¹H NMR (500 MHz, DMSO) δ 10.89 (s, 1H), 8.54 (d, J=6.94 Hz, 1H), 8.03 (s, 1H), 7.69 (br. s., 1H), 7.56 (s, 1H), 7.47 (s, 1H), 7.36-7.25 (m, 2H), 7.23 (d, J=1.50 Hz, 1H), 7.10 (dd, J=7.92, 1.47 Hz, 1H), 5.34 (br. s., 1H), 4.09 (d, J=12.10 Hz, 1H), 4.03-3.91 (m, 1H), 3.76 (s, 3H), 3.06 (t, J=12.08 Hz, 1H), 2.04-1.93 (m, 1H), 1.89-1.73 (m, 2H), 1.72-1.49 (m, 2H), 1.14 (d, J=6.85 Hz, 3H), 1.05-0.93 (m, 2H), 0.80-0.69 (m, 2H).

### Example D-21: Synthesis of 5-[(2R,3R)-3-(5-cyclopropylpyridine-2-amido)-2-methylpiperidin-1-yl]-3-[(1-methyl-1H-pyrazol-4-yl)amino]pyrazine-2-carboxamide (D-21)

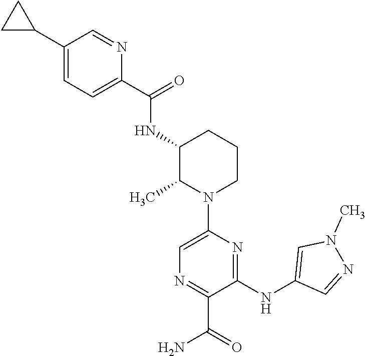 [SMILES: C[C@@H]1[C@H](NC(=O)c2ccc(C3CC3)cn2)CCCN1c1cnc(C(N)=O)c(Nc2cnn(C)c2)n1]

In a similar manner as described in Example D-11, 5-[(2R,3R)-3-(5-cyclopropylpyridine-2-amido)-2-methylpiperidin-1-yl]-3-[(1-methyl-1H-pyrazol-4-yl)amino]pyrazine-2-carboxamide (D-21) was prepared. MS found for C24H29N9O2 as (M+H)⁺ 476.3.

¹H NMR (500 MHz, DMSO) δ 10.87 (s, 1H), 8.56 (d, J=7.00 Hz, 1H), 8.50 (d, J=1.92 Hz, 1H), 8.01 (s, 1H), 7.95 (d, J=8.10 Hz, 1H), 7.70 (br. s., 1H), 7.63 (dd, J=8.23, 2.20 Hz, 1H), 7.57 (s, 1H), 7.47 (s, 1H), 7.28 (br. s., 1H), 5.30 (br. s., 1H), 4.23-3.96 (m, 2H), 3.78 (s, 3H), 3.07 (td, J=13.00, 1.72 Hz, 1H), 2.15-1.97 (m, 2H), 1.92-1.78 (m, 1H), 1.73 (d, J=9.88 Hz, 1H), 1.66-1.54 (m, 1H), 1.20-1.01 (m, 5H), 0.90-0.78 (m, 2H).

### Example D-22: Synthesis of 5-[(2R,3R)-3-(4-tert-butylbenzamido)-2-methylpiperidin-1-yl]-3-[(1-methyl-1H-pyrazol-4-yl)amino]pyrazine-2-carboxamide (D-22)

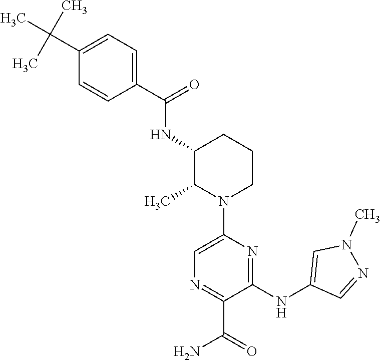 [SMILES: C[C@@H]1[C@H](NC(=O)c2ccc(C(C)(C)C)cc2)CCCN1c1cnc(C(N)=O)c(Nc2cnn(C)c2)n1]

In a similar manner as described in Example D-11, 5-[(2R,3R)-3-(4-tert-butylbenzamido)-2-methylpiperidin-1-yl]-3-[(1-methyl-1H-pyrazol-4-yl)amino]pyrazine-2-carboxamide (D-22) was prepared. MS found for C26H34N8O2 as (M+H)⁺ 491.3.

¹H NMR (500 MHz, DMSO) δ 10.87 (s, 1H), 8.36 (d, J=6.72 Hz, 1H), 8.04 (br. s., 1H), 7.85 (d, J=8.51 Hz, 2H), 7.69 (br. s., 1H), 7.57 (s, 1H), 7.53-7.44 (m, 3H), 7.28 (br. s., 1H), 5.32 (br. s., 1H), 4.31-3.95 (m, 2H), 3.78 (s, 3H), 3.08 (t, J=12.14 Hz, 1H), 1.96 (qd, J=12.99, 3.84 Hz, 1H), 1.86 (d, J=13.17 Hz, 1H), 1.71 (d, J=10.02 Hz, 1H), 1.66-1.54 (m, 1H), 1.31 (s, 9H), 1.09 (d, J=6.86 Hz, 3H).

### Example D-23: Synthesis of 3-[(1-methyl-1H-pyrazol-4-yl)amino]-5-[(2R,3R)-2-methyl-3-[4-(oxetan-3-yl)benzamido]piperidin-1-yl]pyrazine-2-carboxamide (D-23)

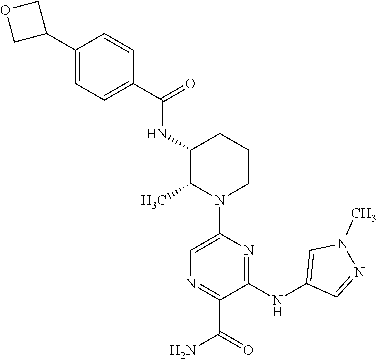 [SMILES: C[C@@H]1[C@H](NC(=O)c2ccc(C3COC3)cc2)CCCN1c1cnc(C(N)=O)c(Nc2cnn(C)c2)n1]

In a similar manner as described in Example D-11, 3-[(1-methyl-1H-pyrazol-4-yl)amino]-5-[(2R,3R)-2-methyl-3-[4-(oxetan-3-yl)benzamido]piperidin-1-yl]pyrazine-2-carboxamide (D-23) was prepared. MS found for C25H30N8O3 as (M+H)⁺ 491.2.

¹H NMR (500 MHz, DMSO) δ 10.87 (s, 1H), 8.43 (d, J=6.80 Hz, 1H), 8.03 (s, 1H), 7.94 (d, J=8.33 Hz, 2H), 7.69 (br. s., 1H), 7.57 (s, 1H), 7.57 (s, 1H), 7.52 (d, J=8.33 Hz, 2H), 7.47 (s, 1H), 7.27 (br. s., 1H), 5.48-5.15 (m, 1H), 4.97 (dd, J=8.55, 5.92 Hz, 2H), 4.64 (t, J=6.25 Hz, 2H), 4.32 (quin, J=7.56 Hz, 1H), 4.21-3.92 (m, 2H), 3.76 (s, 3H), 3.15-3.00 (m, 1H), 2.06-1.82 (m, 2H), 1.77-1.49 (m, 2H), 1.10 (d, J=6.80 Hz, 3H).

### Example D-24: Synthesis of 5-[(2R,3R)-3-[4-(dimethylamino)benzamido]-2-methylpiperidin-1-yl]-3-[(1-methyl-1H-pyrazol-4-yl)amino]pyrazine-2-carboxamide (D-24)

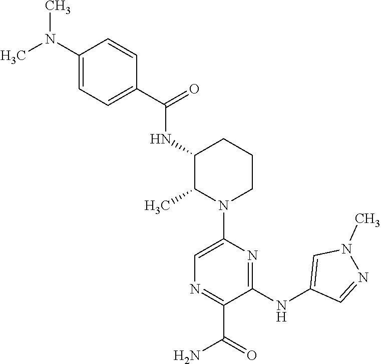 [SMILES: C[C@@H]1[C@H](NC(=O)c2ccc(N(C)C)cc2)CCCN1c1cnc(C(N)=O)c(Nc2cnn(C)c2)n1]

In a similar manner as described in Example 7, 5-[(2R,3R)-3-[4-(dimethylamino)benzamido]-2-methylpiperidin-1-yl]-3-[(1-methyl-1H-pyrazol-4-yl)amino]pyrazine-2-carboxamide (D-24) was prepared. MS found for C24H31N9O2 as (M+H)⁺ 478.5.

¹H NMR (500 MHz, DMSO) δ 10.91-10.80 (m, 1H), 8.05 (d, J=6.85 Hz, 2H), 7.82 (d, J=8.80 Hz, 2H), 7.68 (br. s., 1H), 7.56 (s, 1H), 7.46 (s, 1H), 7.27 (br. s., 1H), 6.72 (d, J=9.29 Hz, 2H), 5.32 (br. s., 1H), 4.10 (br. s., 1H), 4.01 (td, J=11.74, 4.89 Hz, 1H), 3.80-3.75 (m, 3H), 3.08 (t, J=11.98 Hz, 1H), 2.98 (s, 6H), 1.94 (m, J=12.96, 3.67 Hz, 1H), 1.89-1.82 (m, 1H), 1.74-1.66 (m, 1H), 1.65-1.53 (m, 1H), 1.07 (d, J=6.85 Hz, 3H).

### Example D-25: Synthesis of 5-[(2R,3R)-3-[4-cyclopropyl-2-(trifluoromethyl)benzamido]-2-methylpiperidin-1-yl]-3-[(1-methyl-1H-pyrazol-4-yl)amino]pyrazine-2-carboxamide (D-25)

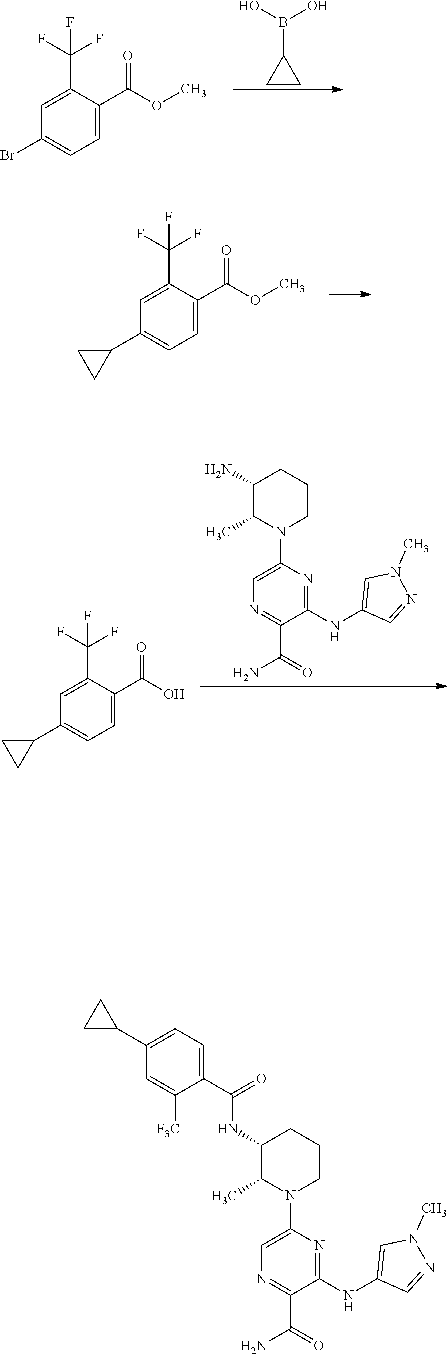 [SMILES: COC(=O)c1ccc(Br)cc1C(F)(F)F ; OB(O)C1CC1 ; COC(=O)c1ccc(C2CC2)cc1C(F)(F)F ; O=C(O)c1ccc(C2CC2)cc1C(F)(F)F ; C[C@@H]1[C@H](N)CCCN1c1cnc(C(N)=O)c(Nc2cnn(C)c2)n1 ; C[C@@H]1[C@H](NC(=O)c2ccc(C3CC3)cc2C(F)(F)F)CCCN1c1cnc(C(N)=O)c(Nc2cnn(C)c2)n1]

To a solution of methyl 4-bromo-2-(trifluoromethyl)benzoate (537 mg, 1.67 mmol) in 13 mL of toluene was added at room temperature cyclopropylboronic acid (220 mg, 2.56 mmol), K₃PO₄ (641 mg, 3.02 mmol), water (0.4 mL) and Pd(Ph₃)₄ (193 mg, 0.167 mmol) and the resulting mixture was degassed for 10 minutes with a stream of N₂. The reaction mixture was stirred at 110° C. for 7 hours. Water was added and product was extracted with ethyl acetate (three times). The collected organic layers were dried over Na₂SO₄, filtered and evaporated in high vacuum to yield methyl 4-cyclopropyl-2-(trifluoromethyl)benzoate (crude, 802 mg) as a grey solid. MS found for C12H11F3O2 as (M+H)⁺ 245.0.

To a solution of methyl 4-cyclopropyl-2-(trifluoromethyl)benzoate (crude, 802 mg) in 2 mL of THF and 2 mL of MeOH was added at room temperature a solution of LiOH.H₂O (140 mg, 3.34 mmol) in 2 mL of water. The reaction mixture was stirred at room temperature overnight. The reaction mixture was quenched with aqueous HCl (6N, 1.5 mL) and the product was extracted with ethyl acetate (twice). The collected organic layers were dried over Na₂SO₄, filtered and evaporated in high vacuum to yield crude 4-cyclopropyl-2-(trifluoromethyl)benzoic acid (247 mg) as a grey solid. MS found for C11H9F3O2 as (M+H)⁺ 231.0.

In a similar manner as described in Example D-11, 5-[(2R,3R)-3-[4-cyclopropyl-2-(trifluoromethyl)benzamido]-2-methylpiperidin-1-yl]-3-[(1-methyl-1H-pyrazol-4-yl)amino]pyrazine-2-carboxamide (D-25) was prepared. MS found for C26H29F3N8O2 as (M+H)⁺ 543.2.

¹H NMR (500 MHz, DMSO) δ 10.90 (s, 1H), 8.60 (d, J=6.85 Hz, 1H), 8.00 (s, 1H), 7.70 (br. s., 1H), 7.55 (s, 1H), 7.50 (s, 1H), 7.47 (s, 1H), 7.44-7.38 (m, 2H), 7.29 (br. s., 1H), 5.39-5.08 (m, 1H), 4.18-4.03 (m, 1H), 4.01-3.93 (m, 1H), 3.73 (s, 3H), 3.06 (t, J=11.98 Hz, 1H), 2.14-2.05 (m, 1H), 1.88-1.75 (m, 2H), 1.71-1.53 (m, 2H), 1.12 (d, J=6.85 Hz, 3H), 1.07-1.00 (m, 2H), 0.81-0.75 (m, 2H).

### Preparation of 4-cyclopropyl-N-(4-hydroxypiperidin-3-yl)benzamide and 4-cyclopropyl-N-(3-hydroxypiperidin-4-yl)benzamide

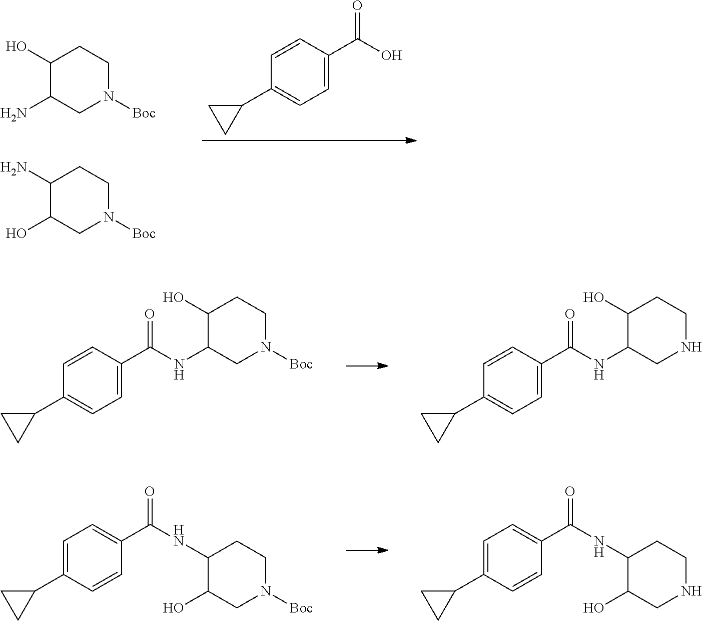

To a mixture of tert-butyl 3-amino-4-hydroxypiperidine-1-carboxylate and tert-butyl 4-amino-3-hydroxypiperidine-1-carboxylate (517 mg, 2.39 mmol) and 4-cyclopropyl-benzoic acid (426 mg. 2.63 mmol) were dissolved in DMF (12 mL), then DIPEA (1.25 mL, 7.17 mmol) and PyBOP (1.55 g, 2.99 mmol) were added and the mixture was stirred at room temperature 2 hours. Water was added and the product was extracted with ethyl acetate (three times). The collected organic layers were dried over Na₂SO₄, filtered and concentrated under high vacuum. The crude obtained was purified by silica flash chromatography to give tert-butyl 3-(4-cyclopropylbenzamido)-4-hydroxypiperidine-1-carboxylate (532 mmol, 52% yield, main isomer) as colorless oil and 4-(4-cyclopropylbenzamido)-3-hydroxypiperidine-1-carboxylate (210 mg, 21% yield) as colorless oil. MS found for C₂₀H₂₈N₂O₄ as (M+H)⁺ 361.1.

To a solution of tert-butyl 4-(4-cyclopropylbenzamido)-3-hydroxypiperidine-1-carboxylate (210 mg, 0.53 mmol) in DCM (6 mL) was added at room temperature TFA (2 mL) and the resulting mixture was stirred at the same temperature for 4.5 hours. The solvent was evaporated and the residue was purified by SCX cartridge to give 4-cyclopropyl-N-(4-hydroxypiperidin-3-yl)benzamide (103 mg, 75% yield) as colorless oil. MS found for C15H20N2O2 as (M+H)⁺ 261.0.

In a similar manner as described in previous procedure 4-cyclopropyl-N-(3-hydroxypiperidin-4-yl)benzamide was prepared. MS found for C15H20N2O2 as (M+H)⁺ 261.0.

### Example D-26: Synthesis of (racemic)-trans-5-[3-(4-cyclopropylbenzamido)-4-hydroxypiperidin-1-yl]-3-[(1-methyl-1H-pyrazol-4-yl)amino]pyrazine-2-carboxamide (D-26)

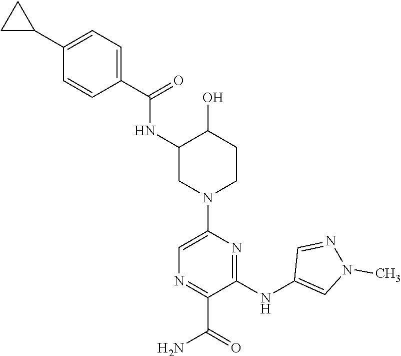 [SMILES: Cn1cc(Nc2nc(N3CCC(O)C(NC(=O)c4ccc(C5CC5)cc4)C3)cnc2C(N)=O)cn1]

In a similar manner as described in Example D-6, 5-[3-(4-cyclopropylbenzamido)-4-hydroxypiperidin-1-yl]-3-[(1-methyl-1H-pyrazol-4-yl)amino]pyrazine-2-carboxamide (D-26) was prepared from 4-cyclopropyl-N-(4-hydroxypiperidin-3-yl)benzamide. MS found for C24H28N8O3 as (M+H)⁺ 477.5.

¹H NMR (400 MHz, DMSO) δ 10.87 (s, 1H), 8.27 (d, J=7.03 Hz, 1H), 8.02 (s, 1H), 7.80 (d, J=8.53 Hz, 2H), 7.71 (br. s., 1H), 7.63 (s, 1H), 7.48 (s, 2H), 7.28 (br. s., 1H), 7.17 (d, J=8.53 Hz, 2H), 5.12-4.97 (m, 1H), 4.64-4.51 (m, 1H), 4.23 (br. s., 2H), 3.88-3.65 (m, 4H), 3.32-3.11 (m, 1H), 2.96-2.81 (m, 1H), 2.13-1.90 (m, 3H), 1.56-1.42 (m, 1H), 1.07-0.94 (m, 2H), 0.81-0.66 (m, 2H).

### Example D-27: Synthesis of 5-[(2R,3R)-3-(5-bromo-1-oxo-2,3-dihydro-1H-isoindol-2-yl)-2-methylpiperidin-1-yl]-3-[(1-methyl-1H-pyrazol-4-yl)amino]pyrazine-2-carboxamide (D-27)

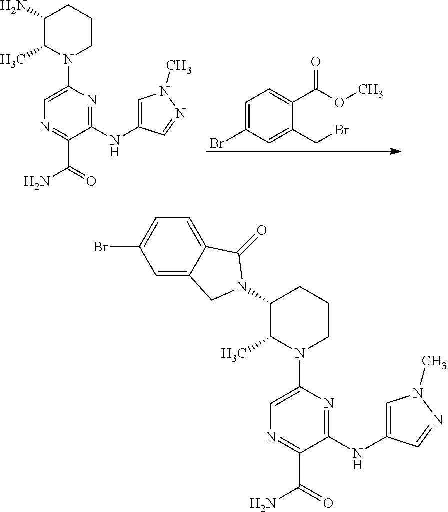 [SMILES: C[C@@H]1[C@H](N)CCCN1c1cnc(C(N)=O)c(Nc2cnn(C)c2)n1 ; COC(=O)c1ccc(Br)cc1CBr ; C[C@@H]1[C@H](N2Cc3cc(Br)ccc3C2=O)CCCN1c1cnc(C(N)=O)c(Nc2cnn(C)c2)n1]

A mixture of 5-[(2R,3R)-3-amino-2-methylpiperidin-1-yl]-3-[(1-methyl-1H-pyrazol-4-yl)amino]pyrazine-2-carboxamide (75 mg, 0.227 mmol), methyl 4-bromo-2-(bromomethyl)benzoate (70 mg, 0.227 mmol) and K₂CO₃ (47 mg, 0.342 mmol) in 3 mL of EtOH was stirred at 45° C. for 34 hours. The mixture was cooled to room temperature, ethyl acetate was added. The solid was filtered off and the organic phase was concentrated. The residue was purified by silica flash chromatography with 0 to 4.5% of MeOH in DCM to give 5-[(2R,3R)-3-(5-bromo-1-oxo-2,3-dihydro-1H-isoindol-2-yl)-2-methylpiperidin-1-yl]-3-[(1-methyl-1H-pyrazol-4-yl)amino]pyrazine-2-carboxamide (57 mg, 35% yield) as a yellow solid (D-27). MS found for C23H25BrN8O2 as (M+H)⁺ 525.4.

¹H NMR (500 MHz, DMSO) δ 10.88 (s, 1H 7.29 (br. s., 1H),), 8.08 (br. s., 1H), 7.90 (s, 1H). 7.76-7.62 (m, 3H), 7.58 (s, 1H), 7.45 (s, 1H), 5.74-5.20 (m, 1H), 4.72-4.51 (m, 2H), 4.26-4.04 (m, 2H), 3.84 (s, 3H), 3.17-3.05 (m, 1H), 2.14 (qd, J=12.81, 3.57 Hz, 1H), 2.05-1.89 (m, 2H), 1.73-1.61 (m, 1H), 1.03 (d, J=6.86 Hz, 3H).

### Example D-28: Synthesis of Trans-5-[4-(4-cyclopropylbenzamido)-3-hydroxypiperidin-1-yl]-3-[(1-methyl-1H-pyrazol-4-yl)amino]pyrazine-2-carboxamide (Enantiomer II) (D-28)

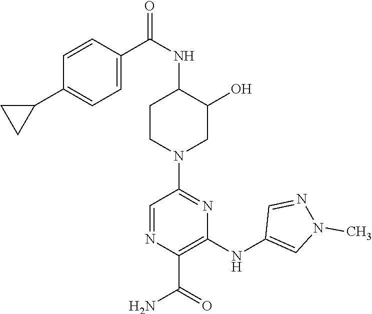 [SMILES: Cn1cc(Nc2nc(N3CCC(NC(=O)c4ccc(C5CC5)cc4)C(O)C3)cnc2C(N)=O)cn1]

In a similar manner as described in Example D-6, followed by chiral-LC, trans-5-[4-(4-cyclopropylbenzamido)-3-hydroxypiperidin-1-yl]-3-[(1-methyl-1H-pyrazol-4-yl)amino]pyrazine-2-carboxamide (Enantiomer 11) (D-28) was prepared using 4-cyclopropyl-N-(3-hydroxypiperidin-4-yl)benzamide. MS found for C24H28N8O3 as (M+H)⁺ 477.1.

¹H NMR (500 MHz, DMSO) δ 10.85 (s, 1H), 8.10 (d, J=8.23 Hz, 1H), 7.86 (s, 1H), 7.77-7.68 (m, 3H), 7.64 (s, 1H), 7.59 (s, 1H), 7.30 (br. s., 1H), 7.13 (d, J=8.51 Hz, 2H), 5.22 (d, J=5.21 Hz, 1H), 4.44 (d, J=10.15 Hz, 1H), 4.29 (d, J=13.45 Hz, 1H), 4.06-3.90 (m, 1H), 3.81 (s, 3H), 3.59 (tt, J=9.64, 4.91 Hz, 1H), 3.16 (t, J=11.80 Hz, 1H), 2.94 (dd, J=12.90, 10.15 Hz, 1H), 2.01-1.88 (m, 2H), 1.60-1.45 (m, 1H), 1.02-0.95 (m, 2H), 0.76-0.66 (m, 2H).

### Example D-29: Synthesis of Trans-5-[4-(4-cyclopropylbenzamido)-3-hydroxypiperidin-1-yl]-3-[(1-methyl-1H-pyrazol-4-yl)amino]pyrazine-2-carboxamide (Enantiomer I) (D-29)

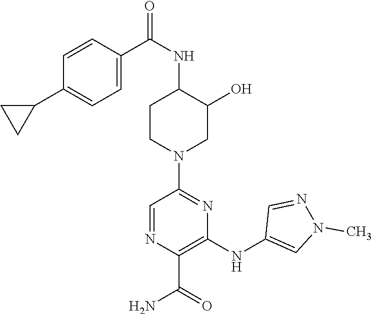 [SMILES: Cn1cc(Nc2nc(N3CCC(NC(=O)c4ccc(C5CC5)cc4)C(O)C3)cnc2C(N)=O)cn1]

In a similar manner as described in Example D-6, followed by chiral-LC, Trans-5-[4-(4-cyclopropylbenzamido)-3-hydroxypiperidin-1-yl]-3-[(1-methyl-1H-pyrazol-4-yl)amino]pyrazine-2-carboxamide (Enantiomer I) (D-29) was prepared. MS found for C24H28N8O3 as (M+H)⁺ 477.1.

¹H NMR (500 MHz, DMSO) δ 10.85 (s, 1H), 8.10 (d, J=8.23 Hz, 1H), 7.86 (s, 1H), 7.77-7.68 (m, 3H), 7.64 (s, 1H), 7.59 (s, 1H), 7.30 (br. s., 1H), 7.13 (d, J=8.51 Hz, 2H), 5.22 (d, J=5.21 Hz, 1H), 4.44 (d, J=10.15 Hz, 1H), 4.29 (d, J=13.45 Hz, 1H), 4.06-3.90 (m, 1H), 3.81 (s, 3H), 3.59 (tt, J=9.64, 4.91 Hz, 1H), 3.16 (t, J=11.80 Hz, 1H), 2.94 (dd, J=12.90, 10.15 Hz, 1H), 2.01-1.88 (m, 2H), 1.60-1.45 (m, 1H), 1.02-0.95 (m, 2H), 0.76-0.66 (m, 2H).

### Example D-30: Synthesis of 5-[(3R,5S)-3-(4-cyclopropylbenzamido)-5-methylpiperidin-1-yl]-3-[(1-methyl-1H-pyrazol-4-yl)amino]pyrazine-2-carboxamide (D-30)

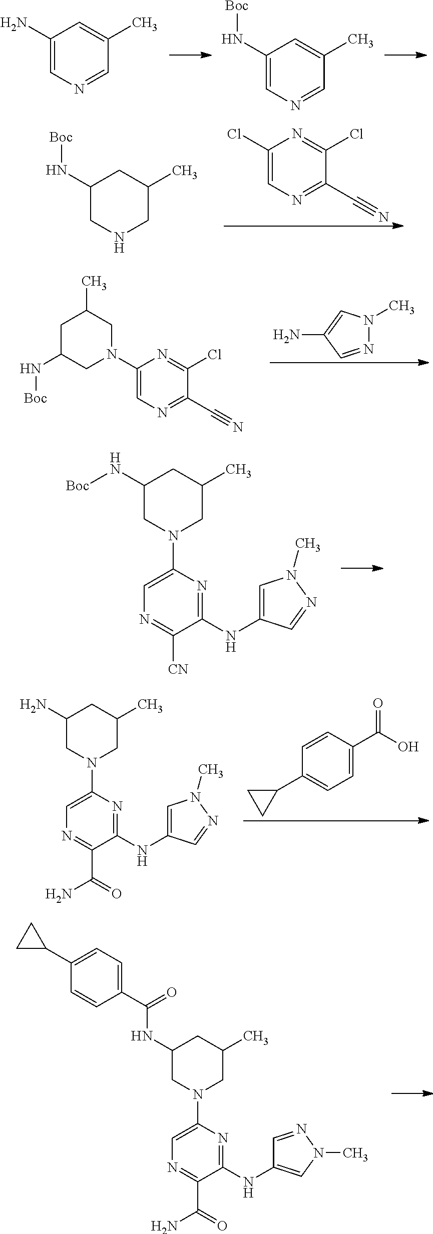

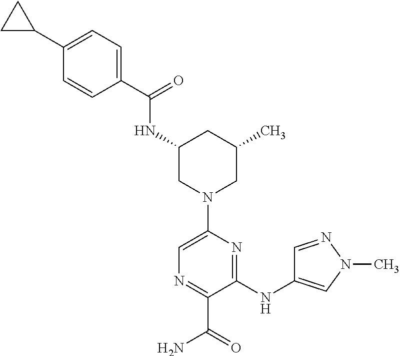 [SMILES: C[C@H]1C[C@@H](NC(=O)c2ccc(C3CC3)cc2)CN(c2cnc(C(N)=O)c(Nc3cnn(C)c3)n2)C1]

To a solution of 3-amino-5-methylpyridine (500 mg, 4.63 mmol) in 8 mL of dry THF was added at room temperature sodium bis(trimethylsilylamide) 1M in THF (10.19 mL, 10.19 mmol) and the resulting mixture was stirred at room temperature for 15 minutes. Then di-tert-butyl dicarbonate (1.10 g, 4.90 mmol) was added at room temperature to the reaction mixture that was stirred at the same temperature overnight. Water was added and the product was extracted with ethyl acetate (three times). The collected organic layers were dried over Na₂SO₄, filtered and concentrated under high vacuum to achieve crude tert-butyl N-(5-methylpyridin-3-yl)carbamate (1.06 g, quant. yield) as orange solid. MS found for C11H16N2O2 as (M+H)⁺ 209.0.

Tert-butyl N-(5-methylpyridin-3-yl)carbamate (crude, 1.06 g, 4.63 mmol) was dissolved in AcOH (20 mL). Platinum on carbon (964 mg, 5%) was added and the mixture was stirred under H₂ pressure (70 psi). Then platinum(IV) oxide (264 mg, 1.16 mmol) was added and the mixture was stirred under H₂ pressure (70 psi) for further 32 hours. The catalyst was filtered off, the solvent evaporated under reduced pressure to give a crude mixture that was further purified by SCX cartridge to give tert-butyl N-(5-methylpiperidin-3-yl)carbamate (0.655 g, 66% yield) as diastereoisomeric mixture. MS found for C11H22N2O2 as (M+H)⁺ 215.1.

3,5-Dichloropyrazine-2-carbonitrile (585 mg, 3.36 mmol), tert-butyl N-(5-methylpiperidin-3-yl)carbamate (655 mg, 3.06 mmol) and DIEA (1.07 mL, 6.12 mmol) were dissolved in EtOH (20 mL) and stirred at 40° C. for 50 minutes. The reaction was concentrated and water was added to the residue. The product was extracted with ethyl acetate (twice). The collected organic layers were dried over Na₂SO₄, filtered and concentrated under high vacuum to give crude tert-butyl N-[1-(6-chloro-5-cyanopyrazin-2-yl)-5-methylpiperidin-3-yl]carbamate (1.18 g, 90% yield) as diastereoisomeric mixture (d.r.=4/11). MS found for C16H22ClN5O2 as (M+H)⁺ 352.0. Tert-butyl N-[1-(6-chloro-5-cyanopyrazin-2-yl)-5-methylpiperidin-3-yl]carbamate (1.18 g, 3.06 mmol), 4-amino-1-methylpyrazole (416 mg, 4.28 mmol) and Cs₂CO₃ (2.99 g, 9.18 mmol) were dissolved in dioxane (44 mL). (+/−) BINAP (380 mg, 0.612 mmol) and Pd(OAc)₂ (140 mg. 0.612 mmol) were added and the mixture was degassed with a stream of N₂ for 10 minutes. The mixture was stirred at 110° C. for 2.5 h. The reaction mixture was cooled to room temperature; water was added and extracted with ethyl acetate (three times). The collected organic layers were dried over Na₂SO₄, filtered and concentrated under high vacuum to give crude mixture that was purified by silica flash chromatography with 10% to 70% ethyl acetate in cyclohexane to give tert-butyl N-(1-{5-cyano-6-[(1-methyl-1H-pyrazol-4-yl)amino]pyrazin-2-yl}-5-methylpiperidin-3-yl)carbamate (0.80 g, 63% yield) as diastereoisomeric mixture. MS found for C20H28N8O2 as (M+H)⁺ 413.5.

Tert-butyl N-(1-{5-cyano-6-[(1-methyl-1H-pyrazol-4-yl)amino]pyrazin-2-yl}-5-methylpiperidin-3-yl)carbamate (0.80 g, 0.552 mmol) was dissolved in TFA (10 mL) and H₂SO₄ (250 μL). The reaction was stirred at 50° C. for 50 minutes. The reaction was concentrated under reduced pressure. The residue was purified by SCX cartridge to give crude 5-(3-amino-5-methylpiperidin-1-yl)-3-[(1-methyl-1H-pyrazol-4-yl)amino]pyrazine-2-carboxamide (650 mg, quant. yield). MS found for C15H22N8O as (M+H)⁺ 331.4.

5-(3-amino-5-methylpiperidin-1-yl)-3-[(1-methyl-1H-pyrazol-4-yl)amino]pyrazine-2-carboxamide (0.771 mmol) and 4-cyclopropyl-benzoic acid (150 mg, 0.925 mmol) were dissolved in DMF (9 mL), then DIPEA (0.40 mL, 2.31 mmol) and TBTU (309 mg, 0.964 mmol) were added and the mixture was stirred at room temperature 16 h. Water was added and the mixture was extracted with ethyl acetate (three times). The collected organic layers were dried over Na₂SO₄, filtered and concentrated. The crude obtained was purified by silica flash chromatography with 0% to 6% of MeOH in DCM to give 291 mg of pure target product as diastereoisomeric mixture that was further purified by chiral-LC to afford 5-[(3R,5S)-3-(4-cyclopropylbenzamido)-5-methylpiperidin-1-yl]-3-[(1-methyl-1H-pyrazol-4-yl)amino]pyrazine-2-carboxamide (83 mg, 23% yield) as a yellow solid (D-30). MS found for C25H30N8O2 as (M+H)⁺ 475.1.

¹H NMR (500 MHz, DMSO) δ 10.86 (s, 1H), 8.33 (d, J=7.83 Hz, 1H), 8.02 (s, 1H), 7.80 (d, J=8.31 Hz, 2H), 7.72-7.59 (m, 2H), 7.48 (s, 1H), 7.29 (br. s., 1H), 7.17 (d, J=8.31 Hz, 2H), 4.88-4.66 (m, 1H), 4.32 (d, J=11.74 Hz, 1H), 4.01-3.85 (m, 1H), 3.74 (s, 3H), 2.68 (t, J=11.74 Hz, 1H), 2.57 (t, J=12.23 Hz, 1H), 2.05-1.91 (m, 2H), 1.82-1.66 (m, 1H), 1.42 (q, J=12.23 Hz, 1H), 1.05-0.90 (m, 5H), 0.79-0.70 (m, 2H).

### Example D-31: Synthesis of 5-[(3R,5R)-3-(4-cyclopropylbenzamido)-5-methylpiperidin-1-yl]-3-[(1-methyl-1H-pyrazol-4-yl)amino]pyrazine-2-carboxamide (D-31)

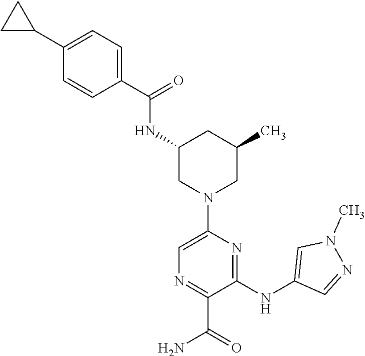 [SMILES: C[C@@H]1C[C@@H](NC(=O)c2ccc(C3CC3)cc2)CN(c2cnc(C(N)=O)c(Nc3cnn(C)c3)n2)C1]

In a similar manner as described in Example D-30, 5-[(3R,5R)-3-(4-cyclopropylbenzamido)-5-methylpiperidin-1-yl]-3-[(1-methyl-1H-pyrazol-4-yl)amino]pyrazine-2-carboxamide (D-31) was prepared. MS found for C25H30N8O2 as (M+H)⁺ 475.1.

¹H NMR (500 MHz, DMSO) δ 10.83 (s, 1H), 8.15 (d, J=6.36 Hz, 1H), 7.95 (s, 1H), 7.61 (d, J=8.31 Hz, 3H), 7.53 (s, 1H), 7.48 (s, 1H), 7.21 (br. s., 1H), 7.08 (d, J=8.31 Hz, 2H), 4.16 (br. s., 1H), 3.99-3.70 (m, 6H), 3.28-3.17 (m, 1H), 2.25 (d, J=3.42 Hz, 1H), 1.98-1.85 (m, 2H), 1.63 (ddd, J=13.21, 8.80, 3.91 Hz, 1H), 1.01-0.92 (m, 5H), 0.73-0.63 (m, 2H).

### Example D-32: Synthesis of 5-[(3S,5R)-3-(4-cyclopropylbenzamido)-5-methylpiperidin-1-yl]-3-[(1-methyl-1H-pyrazol-4-yl)amino]pyrazine-2-carboxamide (D-32)

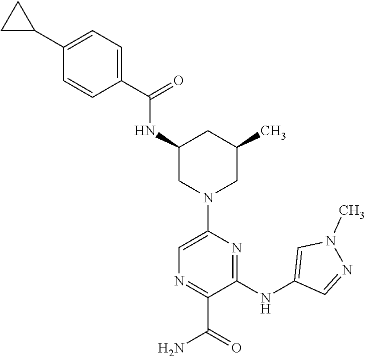 [SMILES: C[C@@H]1C[C@H](NC(=O)c2ccc(C3CC3)cc2)CN(c2cnc(C(N)=O)c(Nc3cnn(C)c3)n2)C1]

In a similar manner as described in Example D-30, 5-[(3S,5R)-3-(4-cyclopropylbenzamido)-5-methylpiperidin-1-yl]-3-[(1-methyl-1H-pyrazol-4-yl)amino]pyrazine-2-carboxamide (D-32) was prepared. MS found for C25H30N8O2 as (M+H)⁺ 475.2.

¹H NMR (500 MHz, DMSO) δ 10.86 (s, 1H), 8.33 (d, J=7.83 Hz, 1H), 8.02 (s, 1H), 7.80 (d, J=8.31 Hz, 2H), 7.73-7.63 (m, 2H), 7.48 (s, 1H), 7.29 (br. s., 1H), 7.17 (d, J=8.31 Hz, 2H), 4.77 (br. s., 1H), 4.32 (d, J=11.74 Hz, 1H), 4.03-3.84 (m, 1H), 3.74 (s, 3H), 2.68 (t, J=11.74 Hz, 1H), 2.57 (t, J=12.23 Hz, 1H), 2.07-1.93 (m, 2H), 1.83-1.69 (m, 1H), 1.42 (q, J=12.23 Hz, 1H), 1.04-0.96 (m, 5H), 0.78-0.71 (m, 2H).

### Example D-33: Synthesis of 5-[(2R,3R)-3-[(dimethylcarbamoyl)amino]-2-methylpiperidin-1-yl]-3-({4-[(1-methylpiperidin-4-yl)oxy]phenyl}amino)pyrazine-2-carboxamide (D-33)

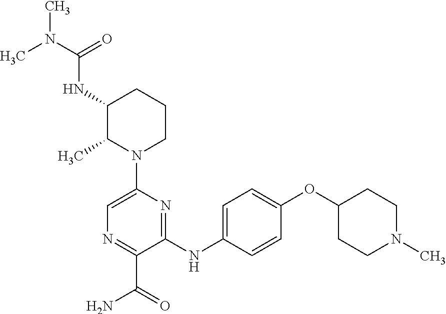

In a similar manner as described in Example 7, 5-[(2R,3R)-3-[(dimethylcarbamoyl)amino]-2-methylpiperidin-1-yl]-3-({4-[(1-methylpiperidin-4-yl)oxy]phenyl}amino)pyrazine-2-carboxamide (D-33) was prepared. MS found for C26H38N8O3 as (M+H)⁺ 511.4.

¹H NMR (500 MHz, DMSO) δ 11.09 (s, 1H), 7.71 (br. s., 1H), 7.57 (s, 1H), 7.50 (d, J=8.78 Hz, 2H), 7.29 (br. s., 1H), 6.88 (d, J=9.06 Hz, 2H), 6.09 (d, J=7.14 Hz, 1H), 5.19-4.67 (m, 1H), 4.34-4.02 (m, 2H), 3.76-3.62 (m, 1H), 3.04-2.94 (m, 1H), 2.85 (s, 6H), 2.59 (br. s., 2H), 2.25-2.06 (m, 5H), 1.96-1.73 (m, 4H), 1.68-1.43 (m, 4H), 1.03 (d, J=6.59 Hz, 3H).

### Example D-34: Synthesis of 5-[(3S,5S)-3-(4-cyclopropylbenzamido)-5-methylpiperidin-1-yl]-3-[(1-methyl-1H-pyrazol-4-yl)amino]pyrazine-2-carboxamide (D-34)

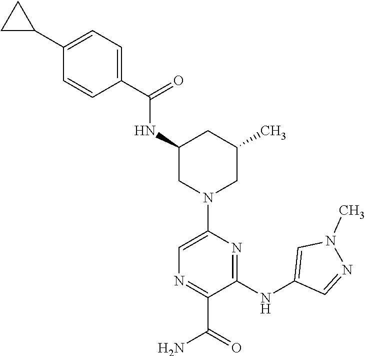 [SMILES: C[C@H]1C[C@H](NC(=O)c2ccc(C3CC3)cc2)CN(c2cnc(C(N)=O)c(Nc3cnn(C)c3)n2)C1]

In a similar manner as described in Example D-30, 5-[(3S,5S)-3-(4-cyclopropylbenzamido)-5-methylpiperidin-1-yl]-3-[(1-methyl-1H-pyrazol-4-yl)amino]pyrazine-2-carboxamide (D-34) was prepared. MS found for C25H30N8O2 as (M+H)⁺ 475.3.

¹H NMR (500 MHz, DMSO) δ 10.83 (s, 1H), 8.15 (d, J=6.36 Hz, 1H), 7.95 (s, 1H), 7.61 (d, J=8.31 Hz, 3H), 7.53 (s, 1H), 7.48 (s, 1H), 7.21 (br. s., 1H), 7.08 (d, J=8.31 Hz, 2H), 4.15 (br. s., 1H), 3.99-3.70 (m, 6H), 3.28-3.18 (m, 1H), 2.31-2.19 (m, 1H), 1.97-1.85 (m, 2H), 1.63 (ddd, J=13.21, 8.80, 3.91 Hz, 1H), 1.00-0.94 (m, 5H), 0.73-0.66 (m, 2H).

### Example D-35: Synthesis of 3-[(1-methyl-1H-pyrazol-4-yl)amino]-5-[(2R,3R)-2-methyl-3-[2-oxo-1-(propan-2-yl)-1,2-dihydropyridine-4-amido]piperidin-1-yl]pyrazine-2-carboxamide (D-35)

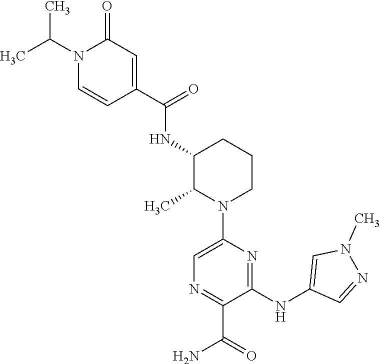 [SMILES: CC(C)n1ccc(C(=O)N[C@@H]2CCCN(c3cnc(C(N)=O)c(Nc4cnn(C)c4)n3)[C@@H]2C)cc1=O]

In a similar manner as described in Example D-11, 3-[(1-methyl-1H-pyrazol-4-yl)amino]-5-[(2R,3R)-2-methyl-3-[2-oxo-1-(propan-2-yl)-1,2-dihydropyridine-4-amido]piperidin-1-yl]pyrazine-2-carboxamide (D-35) was prepared. MS found for C24H31N9O3 as (M+H)⁺ 494.3.

¹H NMR (500 MHz, DMSO) δ 10.87 (s, 1H), 8.60 (d, J=7.02 Hz, 1H), 7.98 (s, 1H), 7.85 (d, J=7.23 Hz, 1H), 7.69 (br. s., 1H), 7.56 (s, 1H), 7.48 (s, 1H), 7.28 (br. s., 1H), 6.87 (d, J=1.75 Hz, 1H), 6.60 (dd, J=7.23, 1.97 Hz, 1H), 5.43-5.13 (m, 1H), 5.05 (quin, J=6.80 Hz, 1H), 4.10 (br. s., 1H), 3.97 (td, J=11.89, 5.37 Hz, 1H), 3.79 (s, 3H), 3.16-3.01 (m, 1H), 2.01-1.50 (m, 4H), 1.30 (d, J=6.80 Hz, 6H), 1.08 (d, J=7.02 Hz, 3H).

### Example D-36: Synthesis of 5-[(2R,3R)-3-(5-cyclopropyl-1-oxo-2,3-dihydro-1H-isoindol-2-yl)-2-methylpiperidin-1-yl]-3-[(1-methyl-1H-pyrazol-4-yl)amino]pyrazine-2-carboxamide (D-36)

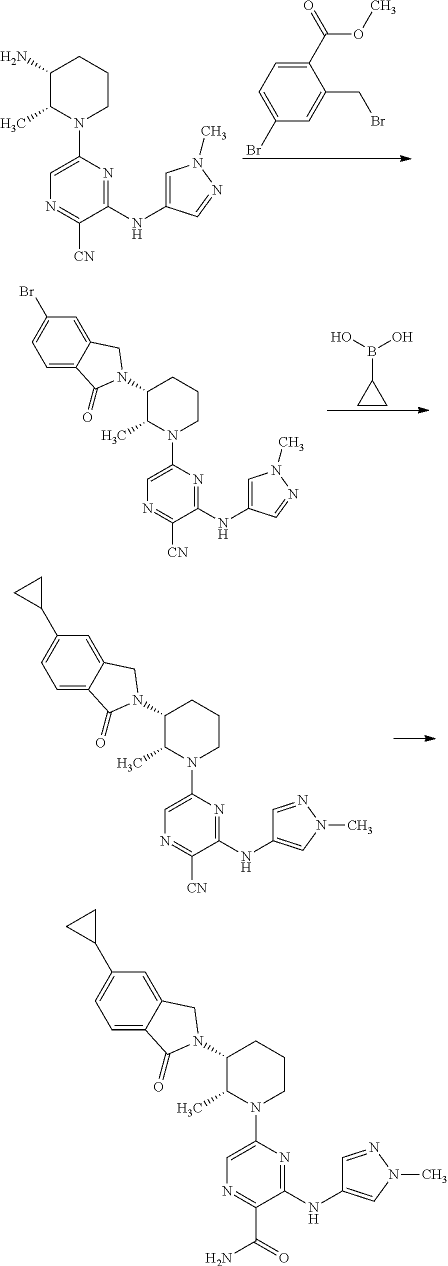 [SMILES: C[C@@H]1[C@H](N)CCCN1c1cnc(C#N)c(Nc2cnn(C)c2)n1 ; COC(=O)c1ccc(Br)cc1CBr ; C[C@@H]1[C@H](N2Cc3cc(Br)ccc3C2=O)CCCN1c1cnc(C#N)c(Nc2cnn(C)c2)n1 ; OB(O)C1CC1 ; C[C@@H]1[C@H](N2Cc3cc(C4CC4)ccc3C2=O)CCCN1c1cnc(C#N)c(Nc2cnn(C)c2)n1 ; C[C@@H]1[C@H](N2Cc3cc(C4CC4)ccc3C2=O)CCCN1c1cnc(C(N)=O)c(Nc2cnn(C)c2)n1]

In a similar manner as described in Example D-27, 5-[(2R,3R)-3-(5-bromo-1-oxo-2,3-dihydro-1H-isoindol-2-yl)-2-methylpiperidin-1-yl]-3-[(1-methyl-1H-pyrazol-4-yl)amino]pyrazine-2-carbonitrile was prepared. MS found for C23H23BrN8O as (M+H)⁺ 507.1.

To a solution of 5-[(2R,3R)-3-(5-bromo-1-oxo-2,3-dihydro-1H-isoindol-2-yl)-2-methylpiperidin-1-yl]-3-[(1-methyl-1H-pyrazol-4-yl)amino]pyrazine-2-carbonitrile (101 mg. 0.20 mmol) in 5 mL of toluene was added cyclopropylboronic acid (26 mg. 0.30 mmol), water (0.2 mL), K₃PO₄ (133 mg, 0.63 mmol) and Pd(PPh₃)₄ (29 mg, 0.025 mmol). The resulting mixture was degassed 10 minutes with a stream of N₂ then was stirred at 110° C. for 30 hours. Water was added and the product was extracted with ethyl acetate (three times). The collected organic layers were dried over Na₂SO₄, filtered and concentrated. The residue obtained was purified by silica flash chromatography with 0% to 4% of MeOH in DCM to give 5-[(2R,3R)-3-(5-cyclopropyl-1-oxo-2,3-dihydro-1H-isoindol-2-yl)-2-methylpiperidin-1-yl]-3-[(1-methyl-1H-pyrazol-4-yl)amino]pyrazine-2-carbonitrile (69 mg, 74% yield) as a yellow solid. MS found for C26H28N8O as (M+H)⁺ 469.0.

To a solution of 5-[(2R,3R)-3-(5-cyclopropyl-1-oxo-2,3-dihydro-1H-isoindol-2-yl)-2-methylpiperidin-1-yl]-3-[(1-methyl-1H-pyrazol-4-yl)amino]pyrazine-2-carbonitrile (69 mg, 0.147 mmol) in 5 mL of MeOH and 0.10 mL of DMSO was added at room temperature a pellet of NaOH (122 mg), 0.20 mL of dry triethylamine and H₂O₂ (0.10 mL, 30% aqueous solution). The mixture was stirred at room temperature for 45 minutes. Then water was added and products were extracted with ethyl acetate (three times). The collected organic layers were dried over Na₂SO₄, filtered and concentrated under high vacuum to give a crude residue that was purified by preparative HPLC to give 5-[(2R,3R)-3-(5-cyclopropyl-1-oxo-2,3-dihydro-1H-isoindol-2-yl)-2-methylpiperidin-1-yl]-3-[(1-methyl-1H-pyrazol-4-yl)amino]pyrazine-2-carboxamide (3.7 mg, 5% yield) as a yellow solid (D-36). MS found for C26H30N8O2 as (M+H)⁺ 487.2.

¹H NMR (400 MHz, DMSO) δ 10.54 (s, 1H), 8.08 (s, 1H), 7.67 (d, J=7.83 Hz, 1H), 7.59 (s, 1H), 7.49 (s, 1H), 7.30 (s, 1H), 7.25 (dd, J=8.22, 1.17 Hz, 1H), 5.66-5.46 (m, 1H), 4.60 (d, J=7.83 Hz, 2H), 4.43-4.30 (m, 1H), 4.20 (d, J=9.39 Hz, 1H), 3.89 (s, 3H), 3.26-3.15 (m, 1H), 2.36-2.18 (m, 1H), 2.12-2.00 (m, 3H), 1.87-1.72 (m, 1H), 1.13 (d, J=7.04 Hz, 3H), 1.08 (dd, J=8.22, 1.96 Hz, 2H), 0.84-0.77 (m, 2H).

### Example D-37: Synthesis of 5-[(2R,3R)-3-(4-cyclopropylbenzamido)-2-methylpiperidin-1-yl]-3-{[4-(piperidin-1-yl)phenyl]amino}pyrazine-2-carboxamide (D-37)

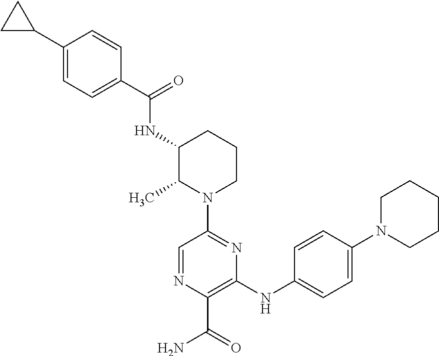 [SMILES: C[C@@H]1[C@H](NC(=O)c2ccc(C3CC3)cc2)CCCN1c1cnc(C(N)=O)c(Nc2ccc(N3CCCCC3)cc2)n1]

In a similar manner as described in Example 40, 5-[(2R,3R)-3-(4-cyclopropylbenzamido)-2-methylpiperidin-1-yl]-3-{[4-(piperidin-1-yl)phenyl]amino}pyrazine-2-carboxamide (D-37) was prepared. MS found for C32H39N7O2 as (M+H)⁺ 554.4.

¹H NMR (500 MHz, DMSO) δ 10.95 (s, 1H), 8.33 (d, J=7.43 Hz, 1H), 7.84 (d, J=8.22 Hz, 2H), 7.70 (br. s., 1H), 7.58 (s, 1H), 7.44 (d, J=9.00 Hz, 2H), 7.27 (br. s., 1H), 7.17 (d, J=8.22 Hz, 2H), 6.79 (d, J=8.80 Hz, 2H), 5.13 (br. s., 1H), 4.28-3.94 (m, 2H), 3.01-3.12 (m, 1H), 3.01-3.12 (m, 1H), 2.91 (br. s., 4H), 2.08-1.77 (m, 3H), 1.74-1.38 (m, 8H), 1.10-0.96 (m, 5H), 0.77-0.68 (m, 2H).

### Example D-38: Synthesis of 3-{[4-(1-cyclopropyl-4-methylpiperidin-4-yl)phenyl]amino}-5-[(2R,3R)-2-methyl-3-(3-methyl-2-oxoimidazolidin-1-yl)piperidin-1-yl]pyrazine-2-carboxamide (D-38)

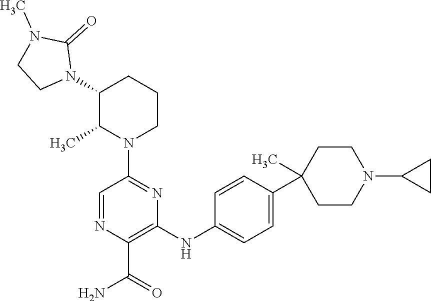 [SMILES: C[C@@H]1[C@H](N2CCN(C)C2=O)CCCN1c1cnc(C(N)=O)c(Nc2ccc(C3(C)CCN(C4CC4)CC3)cc2)n1]

In a similar manner as described in Example 52, 3-{[4-(1-cyclopropyl-4-methylpiperidin-4-yl)phenyl]amino}-5-[(2R,3R)-2-methyl-3-(3-methyl-2-oxoimidazolidin-1-yl)piperidin-1-yl]pyrazine-2-carboxamide (D-38) was prepared using 1-methyl-3-[(2R,3R)-2-methylpiperidin-3-yl]imidazolidin-2-one (prepared according to WO2015084998). MS found for C30H42N8O2 as (M+H)⁺ 547.6.

¹H NMR (400 MHz, DMSO) δ 11.24 (s, 1H), 7.75 (br. s., 1H), 7.61 (s, 1H), 7.56 (d, J=8.77 Hz, 2H), 7.36-7.24 (m, 3H), 5.16-4.89 (m, 1H), 4.28-4.03 (m, 1H), 3.72 (dt, J=12.99, 4.36 Hz, 1H), 3.43-3.35 (m, 2H), 3.30-3.26 (m, 2H), 3.09-2.97 (m, 1H), 2.69 (s, 3H), 2.64-2.53 (m, 3H), 2.48-2.39 (m, 2H), 2.04-1.48 (m, 12H), 1.16 (s, 3H), 1.10 (d, J=7.02 Hz, 3H), 0.41-0.32 (m, 3H), 0.29-0.18 (m, 3H).

### Example D-39: Synthesis of 3-[(3R)-3-[4-(2-hydroxypropan-2-yl)benzamido]piperidin-1-yl]-5-[(1-methyl-1H-pyrazol-4-yl)amino]-1,2,4-triazine-6-carboxamide (D-39)

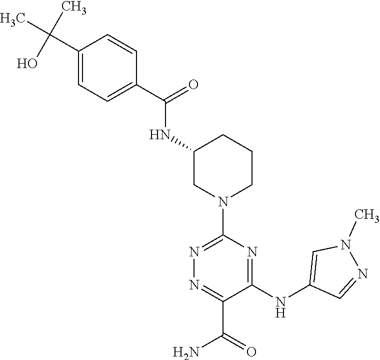 [SMILES: Cn1cc(Nc2nc(N3CCC[C@@H](NC(=O)c4ccc(C(C)(C)O)cc4)C3)nnc2C(N)=O)cn1]

In a similar manner as described in Example D-298, 3-[(3R)-3-[4-(2-hydroxypropan-2-yl)benzamido]piperidin-1-yl]-5-[(I-methyl-1H-pyrazol-4-yl)amino]-1,2,4-triazine-6-carboxamide (D-39) was prepared. MS found for C₂₃H₂₉N₉O₃ as (M+H)⁺ 480.3.

¹H NMR (400 MHz, DMSO) δ 11.00 (s, 1H), 8.43-7.99 (m, 3H), 7.80 (d, J=7.89 Hz, 2H). 7.67 (br. s., 2H), 7.54 (d, J=8.33 Hz, 2H), 5.11 (s, 1H), 4.73 (br. s., 2H), 3.93 (br. s., 1H), 3.85 (s, 3H), 3.27-2.90 (m, 2H), 2.08-1.85 (m, 2H), 1.84-1.51 (m, 2H), 1.43 (s, 6H).

### Example D-40: Synthesis of 5-[(2R,3R)-3-[6-(2-hydroxypropan-2-yl)pyridine-3-amido]-2-methylpiperidin-1-yl]-3-[(1-methyl-1H-pyrazol-4-yl)amino]pyrazine-2-carboxamide (D-40)

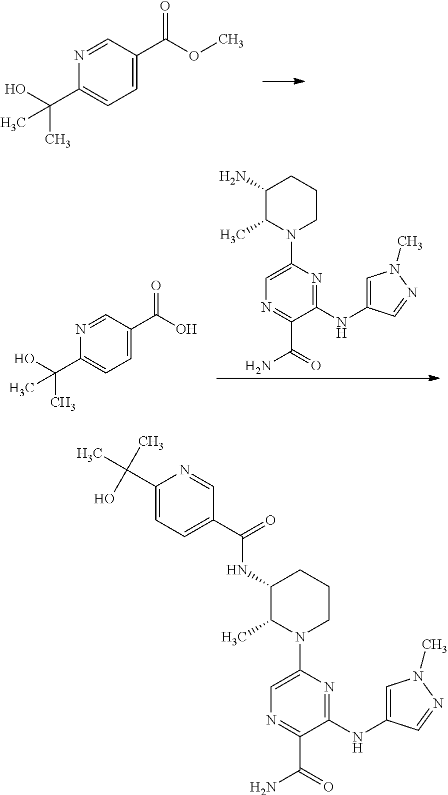 [SMILES: COC(=O)c1ccc(C(C)(C)O)nc1 ; CC(C)(O)c1ccc(C(=O)O)cn1 ; C[C@@H]1[C@H](N)CCCN1c1cnc(C(N)=O)c(Nc2cnn(C)c2)n1 ; C[C@@H]1[C@H](NC(=O)c2ccc(C(C)(C)O)nc2)CCCN1c1cnc(C(N)=O)c(Nc2cnn(C)c2)n1]

To a solution of methyl 6-(2-hydroxypropan-2-yl)pyridine-3-carboxylate (286 mg, 1.47 mmol) in 6 mL of THF was added at room temperature a solution of LiOH.H₂O (123 mg, 2.94 mmol) in 1.5 mL of water. The reaction mixture was stirred at room temperature overnight. The reaction mixture was quenched with aqueous NH₄Cl (20 mL) and aqueous HCl (1N, 3.0 mL) and the product was extracted with ethyl acetate (four times). The collected organic layers were dried over Na₂SO₄, filtered and evaporated in high vacuum to yield 6-(2-hydroxypropan-2-yl)pyridine-3-carboxylic acid (244 mg, 92% yield) as a white solid. MS found for C9H11NO3 as (M+H)⁺ 182.0.

In a similar manner as described in Example D-11, 5-[(2R,3R)-3-[6-(2-hydroxypropan-2-yl)pyridine-3-amido]-2-methylpiperidin-1-yl]-3-[(1-methyl-1H-pyrazol-4-yl)amino]pyrazine-2-carboxamide (D-40) was prepared using 6-(2-hydroxypropan-2-yl)pyridine-3-carboxylic acid and 5-[(2R,3R)-3-amino-2-methylpiperidin-1-yl]-3-[(1-methyl-1H-pyrazol-4-yl)amino]pyrazine-2-carboxamide. MS found for C24H31N9O3 as (M+H)⁺ 494.4.

¹H NMR (500 MHz, DMSO) δ 10.94-10.78 (m, 1H), 8.95 (d, J=1.65 Hz, 1H), 8.58 (d, J=6.59 Hz, 1H), 8.23 (dd, J=8.37, 2.33 Hz, 1H), 8.03 (br. s., 1H), 7.77 (d, J=8.78 Hz, 1H), 7.70 (br. s., 1H), 7.57 (s, 1H), 7.48 (s, 1H), 7.28 (br. s., 1H), 5.34 (s, 1H), 5.56-5.17 (m, 1H), 4.27-3.95 (m, 2H), 3.78 (s, 3H), 3.15-3.02 (m, 1H), 2.03-1.56 (m, 4H), 1.51-1.41 (m, 6H), 1.11 (d, J=6.86 Hz, 3H).

### Example D-41: Synthesis of 3-[(2R,3R)-3-[6-(2-hydroxypropan-2-yl)pyridine-3-amido]-2-methylpiperidin-1-yl]-5-[(1-methyl-1H-pyrazol-4-yl)amino]-1,2,4-triazine-6-carboxamide (D-41)

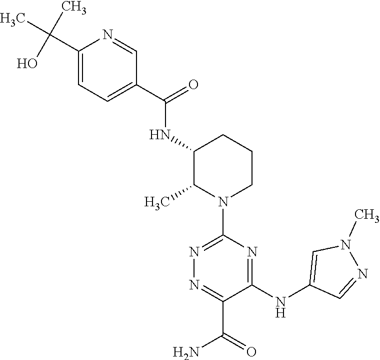 [SMILES: C[C@@H]1[C@H](NC(=O)c2ccc(C(C)(C)O)nc2)CCCN1c1nnc(C(N)=O)c(Nc2cnn(C)c2)n1]

In a similar manner as described in Example D-11, 3-[(2R,3R)-3-[6-(2-hydroxypropan-2-yl)pyridine-3-amido]-2-methylpiperidin-1-yl]-5-[(1-methyl-1H-pyrazol-4-yl)amino]-1,2,4-triazine-6-carboxamide (D-41) was prepared using 6-(2-hydroxypropan-2-yl)pyridine-3-carboxylic acid and 3-[(2R,3R)-3-amino-2-methylpiperidin-1-yl]-5-[(1-methyl-1H-pyrazol-4-yl)amino]-1,2,4-triazine-6-carboxamide. MS found for C23H30N10O3 as (M+H)⁺ 495.0.

¹H NMR (500 MHz, DMSO) δ 11.14-10.85 (m, 1H), 8.96 (br. s., 1H), 8.64-8.47 (m, 1H), 8.36-7.89 (m, 3H), 7.83-7.56 (m, 3H), 5.46 (br. s., 1H), 5.34 (s, 1H), 4.91 (d, J=1.53 Hz, 1H), 4.02 (br. s., 1H), 3.86 (s, 3H), 3.07 (t, J=12.62 Hz, 1H), 2.06-1.52 (m, 4H), 1.46 (s, 6H), 1.13 (d, J=5.76 Hz, 3H).

### Example D-42: Synthesis of 5-[(2R,3R)-3-(4-cyclopropylbenzamido)-2-methylpiperidin-1-yl]-3-{[4-(trifluoromethoxy)phenyl]amino}pyrazine-2-carboxamide (D-42)

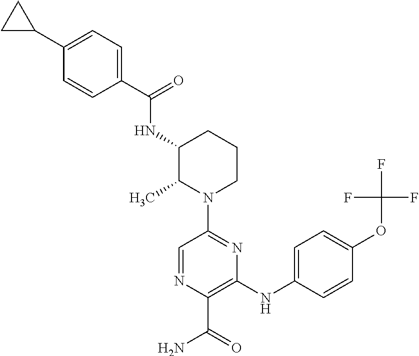 [SMILES: C[C@@H]1[C@H](NC(=O)c2ccc(C3CC3)cc2)CCCN1c1cnc(C(N)=O)c(Nc2ccc(OC(F)(F)F)cc2)n1]

In a similar manner as described in Example 40, 5-[(2R,3R)-3-(4-cyclopropylbenzamido)-2-methylpiperidin-1-yl]-3-{[4-(trifluoromethoxy)phenyl]amino}pyrazine-2-carboxamide (D-42) was prepared. MS found for C28H29F3N603 as (M+H)⁺ 555.4.

¹H NMR (500 MHz, DMSO) δ 11.45 (s, 1H), 8.33 (d, J=7.14 Hz, 1H), 7.87-7.79 (m, 3H), 7.76 (d, J=8.92 Hz, 2H), 7.71 (s, 1H), 7.41 (br. s., 1H), 7.26 (d, 3=8.51 Hz, 2H), 7.17 (d, J=8.23 Hz, 2H), 5.21 (br. s., 1H), 4.40-3.90 (m, 2H), 3.10 (t, J=12.62 Hz, 1H), 2.10-1.78 (m, 3H), 1.76-1.49 (m, 2H), 1.17-0.94 (m, 5H), 0.80-0.66 (m, 2H).

### Example D-43: Synthesis of (racemic)-cis-5-[3-(4-cyclopropylbenzamido)-5-(hydroxymethyl)piperidin-1-yl]-3-[(1-methyl-1H-pyrazol-4-yl)amino]pyrazine-2-carboxamide (D-43)

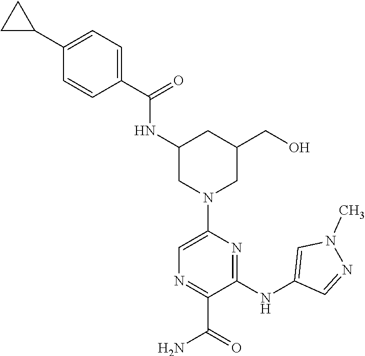 [SMILES: Cn1cc(Nc2nc(N3CC(CO)CC(NC(=O)c4ccc(C5CC5)cc4)C3)cnc2C(N)=O)cn1]

In a similar manner as described in Example 67, (racemic)-cis-5-[3-(4-cyclopropylbenzamido)-5-(hydroxymethyl)piperidin-1-yl]-3-[(1-methyl-1H-pyrazol-4-yl)amino]pyrazine-2-carboxamide carboxamide (D-43) was prepared. MS found for C₂₅H₃₀N₈O₃ as (M+H)⁺ 491.3.

¹H NMR (500 MHz, DMSO) δ 10.88 (s, 1H), 8.36 (d, J=7.41 Hz, 1H), 8.08 (s, 1H), 7.80 (d, J=8.23 Hz, 2H), 7.76-7.66 (m, 1H), 7.61 (s, 1H), 7.47 (s, 1H), 7.32-7.25 (m, 1H), 7.17 (d, J=8.23 Hz, 2H), 4.83 (br. s., 1H), 4.63 (br. s., 2H), 3.92 (d, J=7.41 Hz, 1H), 3.75 (s, 3H), 3.55-3.31 (m, 197H), 2.83-2.71 (m, 1H), 2.70-2.56 (m, 1H), 2.04-1.90 (m, 1H), 1.86-1.74 (m, 1H), 1.47 (q, J=12.17 Hz, 1H), 1.06-0.97 (m, 1H), 0.78-0.70 (m, 1H),

### Example D-44: Synthesis of 5-[(3aR,7aR)-1-(4-cyclopropylbenzoyl)-octahydro-1H-pyrrolo[3,2-b]pyridin-4-yl]-3-[(1-methyl-1H-pyrazol-4-yl)amino]pyrazine-2-carboxamide (D-44)

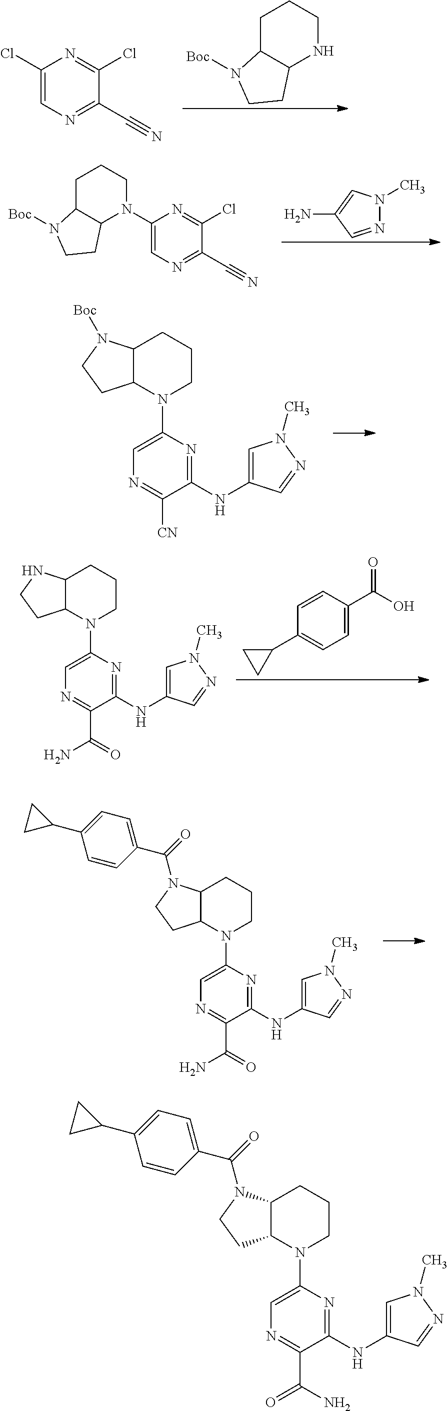

3,5-Dichloropyrazine-2-carbonitrile (202 mg, 1.16 mmol), (racemic)-cis-tert-butyl octahydro-1H-pyrrolo[3,2-b]pyridine-1-carboxylate (250 mg, 1.105 mmol) and DIEA (0.770 mL. 4.42 mmol) were dissolved in EtOH (10 mL) and stirred at 40° C. for 45 minutes. The reaction mixture was diluted with ethyl acetate and washed with aqueous solution of NaHCO₃ and with water. The organic phase was dried over Na₂SO₄, filtered and concentrated under high vacuum to give tert-butyl-4-(6-chloro-5-cyanopyrazin-2-yl)-octahydro-1H-pyrrolo[3,2-b]pyridine-1-carboxylate (477 mg, quant. yield) as racemic-cis mixture. MS found for C17H22ClN5O2 as (M+H)⁺ 364.2.

Tert-butyl-4-(6-chloro-5-cyanopyrazin-2-yl)-octahydro-1H-pyrrolo[3,2-b]pyridine-1-carboxylate (crude, 477 mg, 1.105 mmol), 4-amino-1-methylpyrazole (161 mg, 1.66 mmol) and Cs₂CO₃ (1.08 g, 3.32 mmol) were dissolved in dioxane (44 mL). (+/−) BINAP (138 mg, 0.221 mmol) and Pd(OAc)₂ (53 mg, 0.236 mmol) were added and the mixture was degassed with a stream of N₂ for 10 minutes. The mixture was stirred at 70° C. for 1.5 hours, then at 110° C. for further 1 hour. The reaction mixture was cooled to room temperature, water was added and products were extracted with ethyl acetate (three times). The collected organic layers were dried over Na₂SO₄, filtered and concentrated under high vacuum to give a crude mixture that was purified by silica flash chromatography with 20% to 95% of ethyl acetate in cyclohexane to give tert-butyl-4-{5-cyano-6-[(1-methyl-1H-pyrazol-4-yl)amino]pyrazin-2-yl}-octahydro-1H-pyrrolo[3,2-b]pyridine-1-carboxylate (367 mg, 71% yield) as racemic-cis mixture. MS found for C21H28N8O2 as (M+H)⁺ 425.4.

Tert-butyl-4-{5-cyano-6-[(1-methyl-1H-pyrazol-4-yl)amino]pyrazin-2-yl}-octahydro-1H-pyrrolo[3,2-b]pyridine-1-carboxylate (367 g, 0.867 mmol) was dissolved in TFA (5 mL) and H₂SO₄ (120 μL). The reaction was stirred at room temperature for 2.5 hours. The reaction was concentrated under reduced pressure. The residue was purified by SCX cartridge to give 3-[(1-methyl-1H-pyrazol-4-yl)amino]-5-{octahydro-1H-pyrrolo[3,2-b]pyridin-4-yl}pyrazine-2-carboxamide (224 mg, 76% yield) racemic-cis mixture as a yellow solid. MS found for C16H22N8O as (M+H)⁺ 343.3.

3-[(1-Methyl-1H-pyrazol-4-yl)amino]-5-{octahydro-1H-pyrrolo[3,2-b]pyridin-4-yl}pyrazine-2-carboxamide (118 mg, 0.347 mmol) and 4-cyclopropyl-benzoic acid (79 mg, 0.485 mmol) were dissolved in DMF (4 mL), then DIPEA (0.241 mL, 1.39 mmol) and TBTU (167 mg. 0.52 mmol) were added and the mixture was stirred at room temperature for 1 hour. Water was added and the mixture was extracted with ethyl acetate (three times). The collected organic layers were dried over Na₂SO₄, filtered and concentrated. The crude obtained was purified by silica flash chromatography with 0% to 5% of MeOH in DCM to give 105 mg of the target product as racemic mixture that was further purified by chiral-LC to afford 5-[(3aR,7aR)-1-(4-cyclopropylbenzoyl)-octahydro-1H-pyrrolo[3,2-b]pyridin-4-yl]-3-[(1-methyl-1H-pyrazol-4-yl)amino]pyrazine-2-carboxamide (42.5 mg, 25% yield) as a yellow solid (D-44). MS found for C26H30N8O2 as (M+H)⁺ 487.1.

¹H NMR (500 MHz, DMSO) δ 10.82 (br. s., 1H), 7.90-7.81 (m, 1H), 7.71 (br. s., 1H), 7.64 (s, 1H), 7.56 (m, J=8.30 Hz, 1H), 7.42-7.34 (m, 2H), 7.31 (br. s., 1H), 7.18-7.09 (m, 2H), 5.14-4.74 (m, 1H), 4.42-3.79 (m, 2H), 3.80 (s, 3H), 3.71-3.36 (m, 2H), 3.11-2.82 (m, 1H), 2.29-1.16 (m, 7H), 0.98 (d, J=5.87 Hz, 2H), 0.71 (br. s., 2H).

### Example D-45: Synthesis of 5-[(3aS,7aS)-1-(4-cyclopropylbenzoyl)-octahydro-1H-pyrrolo[3,2-b]pyridin-4-yl]-3-[(1-methyl-1H-pyrazol-4-yl)amino]pyrazine-2-carboxamide (D-45)

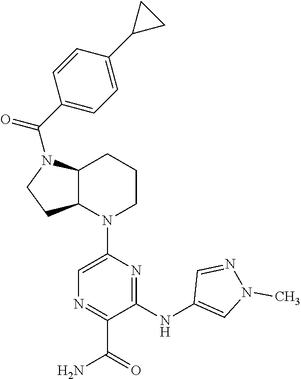 [SMILES: Cn1cc(Nc2nc(N3CCC[C@H]4[C@@H]3CCN4C(=O)c3ccc(C4CC4)cc3)cnc2C(N)=O)cn1]

In a similar manner as described in Example D-44, 5-[(3aS,7aS)-1-(4-cyclopropylbenzoyl)-octahydro-1H-pyrrolo[3,2-b]pyridin-4-yl]-3-[(1-methyl-1H-pyrazol-4-yl)amino]pyrazine-2-carboxamide (D-45). MS found for C26H30N8O2 as (M+H)⁺ 487.1.

¹H NMR (500 MHz, DMSO) δ 10.82 (br. s., 1H), 7.90-7.81 (m, 1H), 7.71 (br. s., 1H), 7.64 (s, 1H), 7.56 (m, J=8.30 Hz, 1H), 7.42-7.34 (m, 2H), 7.31 (br. s., 1H), 7.18-7.09 (m, 2H), 5.14-4.74 (m, 1H), 4.42-3.79 (m, 2H), 3.80 (s, 3H), 3.71-3.36 (m, 2H), 3.11-2.82 (m, 1H), 2.29-1.16 (m, 7H), 0.98 (d, J=5.87 Hz, 2H), 0.71 (br. s., 2H).

### Example D-46: Synthesis of 5-[(4aR,8aR)-5-(4-cyclopropylbenzoyl)-decahydro-1,5-naphthyridin-1-yl]-3-[(1-methyl-1H-pyrazol-4-yl)amino]pyrazine-2-carboxamide (D-46)

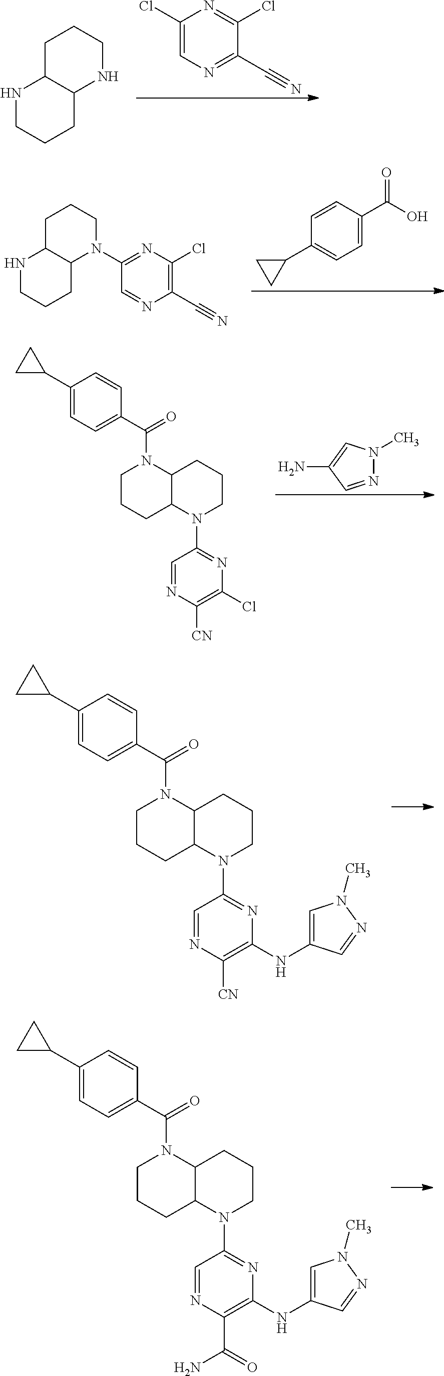

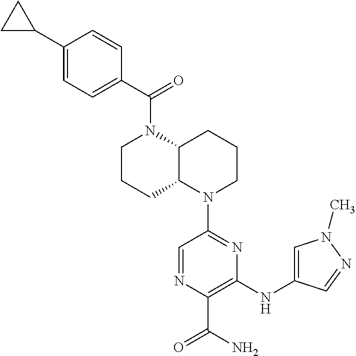 [SMILES: Cn1cc(Nc2nc(N3CCC[C@@H]4[C@H]3CCCN4C(=O)c3ccc(C4CC4)cc3)cnc2C(N)=O)cn1]

3,5-Dichloropyrazine-2-carbonitrile (155 mg, 0.891 mmol). (racemic)-cis-decahydro-1,5-naphthyridine (250 mg, 1.78 mmol) and DIEA (0.155 mL, 0.891 mmol) were dissolved in EtOH (23 mL) and stirred at 35° C. for 3 hours. The reaction mixture was concentrated under high vacuum and the residue was purified by silica-NH flash chromatography (eluent from 100% ethyl acetate to 1% of MeOH, 9% of DCM and 90% of ethyl acetate) to achieve 3-chloro-5-(decahydro-1,5-naphthyridin-1-yl)pyrazine-2-carbonitrile (123 mg, 50% yield) racemic-cis mixture as a white solid. MS found for C13H16ClN5 as (M+H)⁺ 278.2.

3-chloro-5-(decahydro-1,5-naphthyridin-1-yl)pyrazine-2-carbonitrile (123 mg, 0.443 mmol) and 4-cyclopropyl-benzoic acid (88 mg, 0.543 mmol) were dissolved in DMF (5 mL), then DIPEA (0.390 mL, 1.77 mmol) and TBTU (183 mg, 0.570 mmol) were added and the mixture was stirred at room temperature for 50 minutes. Water was added and the mixture was extracted with ethyl acetate (twice). The collected organic layers were dried over Na₂SO₄, filtered and concentrated. The crude obtained was purified by silica flash chromatography with 0% to 75% of ethyl acetate in cyclohexane to give 3-chloro-5-[5-(4-cyclopropylbenzoyl)-decahydro-1,5-naphthyridin-1-yl]pyrazine-2-carbonitrile (105 mg, 59% yield) racemic-cis mixture as a yellowish solid. MS found for C23H24ClN5O as (M+H)⁺ 422.1.

3-Chloro-5-[5-(4-cyclopropylbenzoyl)-decahydro-1,5-naphthyridin-1-yl]pyrazine-2-carbonitrile (110 mg, 0.261 mmol), 4-amino-1-methylpyrazole (45 mg, 0.463 mmol) and Cs₂CO₃ (357 mg, 1.10 mmol) were dissolved in dioxane (6 mL). (+/−) BINAP (32 mg, 0.052 mmol) and Pd(OAc)₂ (18 mg, 0.052 mmol) were added and the mixture was degassed with a stream of N₂ for 10 minutes. The mixture was stirred at 110° C. for 1.5 hours. The reaction mixture was cooled to room temperature, water was added and products were extracted with ethyl acetate (three times). The collected organic layers were dried over Na₂SO₄, filtered and concentrated under high vacuum to give a crude mixture that was purified by silica flash chromatography with 0% to 5% of MeOH in DCM to give 5-[5-(4-cyclopropylbenzoyl)-decahydro-1,5-naphthyridin-1-yl]-3-[(1-methyl-1H-pyrazol-4-yl)amino]pyrazine-2-carbonitrile (108 mg, 86% yield) racemic-cis mixture as a red oil. MS found for C27H30N8O as (M+H)⁺ 483.2.

To a solution of 5-[5-(4-cyclopropylbenzoyl)-decahydro-1,5-naphthyridin-1-yl]-3-[(1-methyl-1H-pyrazol-4-yl)amino]pyrazine-2-carbonitrile (108 mg, 0.224 mmol) in 6 mL of MeOH and 0.10 mL of DMSO was added at room temperature a pellet of NaOH (176 mg), 0.20 mL of dry triethylamine and H₂O₂ (0.10 mL, 30% aqueous solution). The mixture was stirred at room temperature for 40 minutes. Then water was added and products were extracted with ethyl acetate (three times). The collected organic layers were dried over Na₂SO₄, filtered and concentrated under high vacuum to give a crude residue that was purified by SCX cartridge to give 76 mg of the target product as racemate that was further purified by chiral-LC to give 5-[(4aR,8aR)-5-(4-cyclopropylbenzoyl)-decahydro-1,5-naphthyridin-1-yl]-3-[(1-methyl-1H-pyrazol-4-yl)amino]pyrazine-2-carboxamide (33 mg, 29% yield) as a yellow solid. (D-46). MS found for C27H32N8O2 as (M+H)⁺ 501.2.

¹H NMR (500 MHz, DMSO) δ 10.94-10.64 (m, 1H), 7.97-7.46 (m, 3H), 7.40-6.88 (m, 4H), 3.81 (s, 3H), 5.07-2.70 (m, 6H), 2.29-1.28 (m, 9H), 1.08-0.52 (m, 4H).

### Example D-47: Synthesis of 5-[(4aS,8aS)-5-(4-cyclopropylbenzoyl)-decahydro-1,5-naphthyridin-1-yl]-3-[(1-methyl-1H-pyrazol-4-yl)amino]pyrazine-2-carboxamide (D-47)

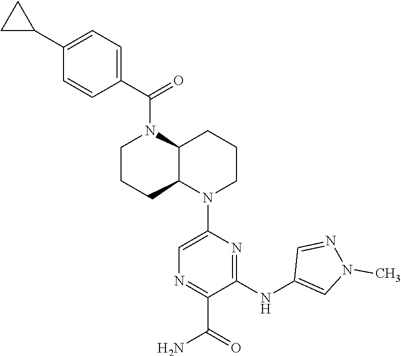 [SMILES: Cn1cc(Nc2nc(N3CCC[C@H]4[C@@H]3CCCN4C(=O)c3ccc(C4CC4)cc3)cnc2C(N)=O)cn1]

In a similar manner as described in Example D46, 5-[(4aS,8aS)-5-(4-cyclopropylbenzoyl)-decahydro-1,5-naphthyridin-1-yl]-3-[(1-methyl-1H-pyrazol-4-yl)amino]pyrazine-2-carboxamide carboxamide (D-47). MS found for C₂₇H₃₂N₈O₂ as (M+H)⁺ 501.1.

¹H NMR (500 MHz, DMSO) δ 10.94-10.64 (m, 1H), 7.97-7.46 (m, 3H), 7.40-6.88 (m, 4H). 3.81 (s, 3H), 5.07-2.70 (m, 6H), 2.29-1.28 (m, 9H), 1.08-0.52 (m, 4H).

### Preparation of 4-[(4,4-difluorocyclohexyl)oxy]aniline

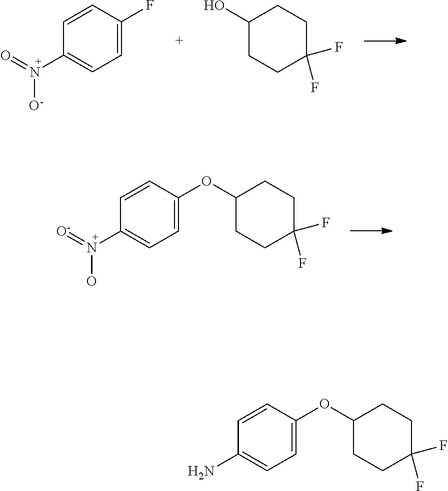

To a solution of 4,4-difluorocyclohexan-1-ol (368 mg, 2.70 mmol) in 14 mL of dry THF was added at room temperature NaH (116 mg, 2.84 mmol, 60% dispersion in mineral oil). The mixture was stirred at room temperature for 5 minutes then 1-fluoro-4-nitrobenzene (0.301 mL, 2.84 mmol) was added and the reaction was stirred at room temperature for 4 hours. Then more NaH was added (95 mg. 60% dispersion in mineral oil) and the mixture was stirred at room temperature for further 2 hours. Et₂O was added to the reaction mixture. The organic phase was washed with water (twice), dried over Na₂SO₄, filtered and concentrated. The residue obtained was purified by silica flash chromatography with 0% to 15% of ethyl acetate in cyclohexane to give 1-[(4,4-difluorocyclohexyl)oxy]-4-nitrobenzene (685 mg, 80% yield) as a yellow solid. MS found for C12H13F2NO3 as (M+H)⁺ 258.0.

To a solution of 1-[(4,4-difluorocyclohexyl)oxy]-4-nitrobenzene (0.685 g, 2.16 mmol) in 50 mL of EtOH was added palladium on carbon (0.230 g, 0.216 mmol, 10% wt.) and the mixture was stirred under hydrogen atmosphere (1 atm) at room temperature overnight. The solid was filtered off, the solution was concentrated to give 4-[(4,4-difluorocyclohexyl)oxy]aniline (515 mg, 87% yield) as a grey oil. MS found for C12H15F2NO as (M+H)⁺ 228.0.

### Example D-48: Synthesis of 5-[(2R,3R)-3-(6-cyclopropylpyridine-3-amido)-2-methylpiperidin-1-yl]-3-({4-[(4,4-difluorocyclohexyl)oxy]phenyl}amino)pyrazine-2-carboxamide (D-48)

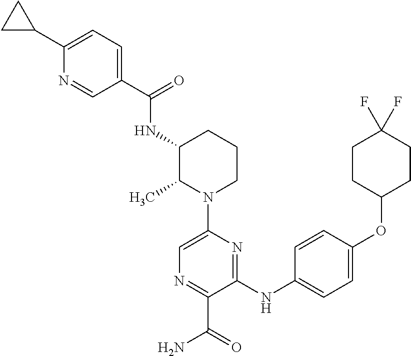 [SMILES: C[C@@H]1[C@H](NC(=O)c2ccc(C3CC3)nc2)CCCN1c1cnc(C(N)=O)c(Nc2ccc(OC3CCC(F)(F)CC3)cc2)n1]

In a similar manner as described in Example D-11, 5-[(2R,3R)-3-(6-cyclopropylpyridine-3-amido)-2-methylpiperidin-1-yl]-3-({4-[(4,4-difluorocyclohexyl)oxy]phenyl}amino)pyrazine-2-carboxamide (D-48) was prepared. MS found for C32H37F2N7O3 as (M+H)⁺ 606.4.

¹H NMR (500 MHz, DMSO) δ 11.05 (s, 1H), 8.89 (s, 1H), 8.49 (d, J=7.43 Hz, 1H), 8.11 (dd, J=8.22, 1.96 Hz, 1H), 7.73 (br. s., 1H), 7.62 (s, 1H), 7.51 (d, J=9.00 Hz, 2H), 7.41 (d, J=8.22 Hz, 1H), 7.30 (br. s., 1H), 6.87 (d, J=8.61 Hz, 2H), 5.32-4.84 (m, 1H), 4.34 (br. s., 1H), 4.23-3.97-4.23 (m, 2H), 3.06 (t, J=12.13 Hz, 1H), 2.24-2.13 (m, 1H), 2.06-1.54 (m, 12H), 1.13-0.87 (m, 7H).

### Example D-49: Synthesis of (racemic)-trans-5-[3-(4-cyclopropylbenzamido)-5-(hydroxymethyl)piperidin-1-yl]-3-[(1-methyl-1H-pyrazol-4-yl)amino]pyrazine-2-carboxamide (D-49)

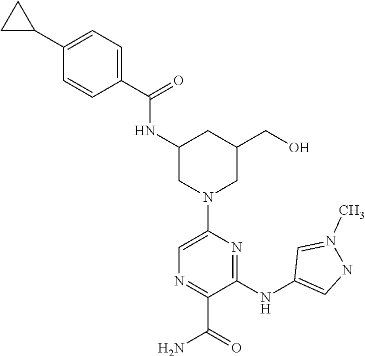

In a similar manner as described in Example 67, (racemic)-trans-5-[3-(4-cyclopropylbenzamido)-5-(hydroxymethyl)piperidin-1-yl]-3-[(1-methyl-1H-pyrazol-4-yl)amino]pyrazine-2-carboxamide (D-49). MS found for C₂₅H₃₀N₈O₃ as (M+H)⁺ 491.1.

¹H NMR (400 MHz, DMSO) δ 8.04 (s, 1H), 7.55 (s, 1H), 7.50 (d, J=8.22 Hz, 2H), 7.46 (s, 1H), 7.06 (d, J=7.72 Hz, 2H), 4.30-4.17 (m, 2H), 4.11 (dd, J=13.30, 5.48 Hz, 1H), 3.82 (s, 3H), 3.88-3.81 (m, 1H), 3.65-3.57 (m, 1H), 3.56-3.48 (m, 1H), 3.47-3.38 (m, 1H), 2.26 (m, J=4.30 Hz, 1H), 2.07-1.77 (m, 3H), 1.00 (dd, J=8.61, 2.35 Hz, 2H), 0.74-0.69 (m, 2H).

### Example D-50: Synthesis of (racemic)-cis-5-[1-(dimethylcarbamoyl)-octahydro-1H-pyrrolo[3,2-b]pyridin-4-yl]-3-[(1-methyl-1H-pyrazol-4-yl)amino]pyrazine-2-carboxamide (D-50)

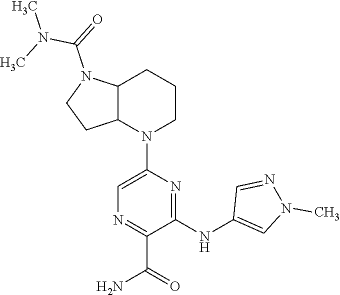

In a similar manner as described in Example 7. (racemic)-cis-5-[1-(dimethylcarbamoyl)-octahydro-1H-pyrrolo[3,2-b]pyridin-4-yl]-3-[(1-methyl-1H-pyrazol-4-yl)amino]pyrazine-2-carboxamide (D-50) was prepared from 3-[(1-methyl-1H-pyrazol-4-yl)amino]-5-{octahydro-1H-pyrrolo[3,2-b]pyridin-4-yl}pyrazine-2-carboxamide. MS found for C19H27N9O2 as (M+H)⁺ 414.1.

¹H NMR (400 MHz, DMSO) δ 10.81 (s, 1H), 7.83 (s, 1H), 7.69 (br. s., 1H), 7.61 (s, 1H), 7.55 (s, 1H), 7.29 (br. s., 1H), 4.81-4.68 (m, 1H), 4.21-4.02 (m, 2H), 3.79 (s, 3H), 3.63-3.53 (m, 1H), 3.46-3.25 (m, 1H), 3.14-2.96 (m, 1H), 2.78 (s, 6H), 2.22-2.05 (m, 1H), 2.04-1.90 (m, 1H), 1.89-1.71 (m, 2H), 1.40-1.57 (m, 2H).

### Example D-51: Synthesis of 5-[(2R,3R)-3-[4-(2-aminopropan-2-yl)benzamido]-2-methylpiperidin-1-yl]-3-[(1-methyl-1H-pyrazol-4-yl)amino]pyrazine-2-carboxamide (D-51)

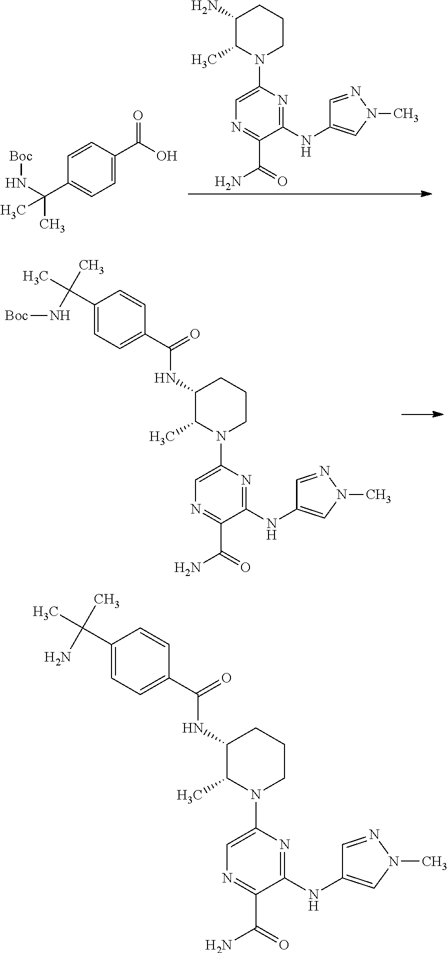

In a similar manner as described in Example D-11, 5-[(2R,3R)-3-[4-(2-aminopropan-2-yl)benzamido]-2-methylpiperidin-1-yl]-3-[(1-methyl-1H-pyrazol-4-yl)amino]pyrazine-2-carboxamide was prepared from 4-(2-{[(tert-butoxy)carbonyl]amino}propan-2-yl)benzoic acid and 5-[(2R,3R)-3-amino-2-methylpiperidin-1-yl]-3-[(1-methyl-1H-pyrazol-4-yl)amino]pyrazine-2-carboxamide. MS found for C30H41N9O4 as (M+H)⁺ 592.2.

To a solution of 5-[(2R,3R)-3-[4-(2-aminopropan-2-yl)benzamido]-2-methylpiperidin-1-yl]-3-[(1-methyl-1H-pyrazol-4-yl)amino]pyrazine-2-carboxamide (39 mg, 0.0659) in 3.5 mL of dry DCM was added at room temperature TFA neat. The mixture was stirred at room temperature for 16 hours. The reaction was concentrated under reduced pressure. The residue was purified by SCX cartridge to give a crude product that was further purified by preparative HPLC to give 5-[(2R,3R)-3-[4-(2-aminopropan-2-yl)benzamido]-2-methylpiperidin-1-yl]-3-[(1-methyl-1H-pyrazol-4-yl)amino]pyrazine-2-carboxamide (19.2 mg, 59% yield) as a yellow solid (D-51). MS found for C₂₅H₃₃N₉O₂ as (M+H)⁺ 492.3.

¹H NMR (500 MHz, DMSO) δ 10.88 (s, 1H), 8.35 (d, J=6.59 Hz, 1H), 8.04 (br. s., 1H), 7.85 (d, J=8.51 Hz, 2H), 7.69 (br. s., 1H), 7.63 (d, J=8.51 Hz, 2H), 7.57 (s, 1H), 7.47 (s, 1H), 7.28 (br. s., 1H), 5.66-5.10 (m, 1H), 4.28-3.95 (m, 2H), 3.78 (s, 3H), 3.09 (t, J=12.21 Hz, 1H), 2.05-1.54 (m, 6H), 1.38 (s, 6H), 1.09 (d, J=6.59 Hz, 3H).

### Example D-52: Synthesis of 5-[(2R,3R)-3-[(dimethylcarbamoyl)amino]-2-methylpiperidin-1-yl]-3-{[2-fluoro-4-(4-methylpiperazin-1-yl)phenyl]amino}pyrazine-2-carboxamide (D-52)

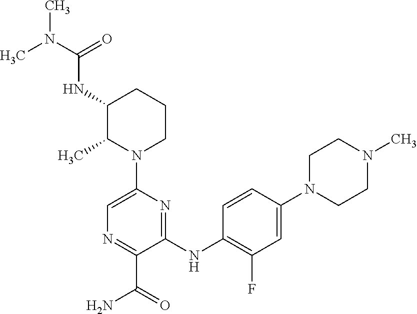

In a similar manner as described in Example 7, 5-[(2R,3R)-3-[(dimethylcarbamoyl)amino]-2-methylpiperidin-1-yl]-3-{[2-fluoro-4-(4-methylpiperazin-1-yl)phenyl]amino}pyrazine-2-carboxamide (D-52) was prepared. MS found for C25H36FN9O2 as (M+H)⁺ 514.3.

¹H NMR (500 MHz, CDCl₃) δ 10.72 (br. s., 1H), 8.07 (t, J=9.05 Hz, 1H), 7.63-7.34 (m, 2H). 6.79-6.62 (m, 2H), 5.14 (br. s., 1H), 4.89 (br. s., 1H), 4.36 (d, J=12.72 Hz, 1H), 4.20 (d, J=6.36 Hz, 1H), 3.98 (d, J=4.40 Hz, 1H), 3.17 (d, J=4.40 Hz, 4H), 2.99-2.82 (m, 7H), 2.66-2.49 (m, 4H), 2.36 (br. s., 3H), 1.93-1.21 (m, 4H), 1.14 (d, J=6.36 Hz, 3H).

### Example D-53: Synthesis of 5-[(2R,3R)-3-[(dimethylcarbamoyl)amino]-2-methylpiperidin-1-yl]-3-[(1H-pyrazol-4-yl)amino]pyrazine-2-carboxamide (D-53)

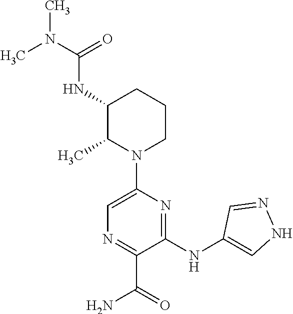 [SMILES: C[C@@H]1[C@H](NC(=O)N(C)C)CCCN1c1cnc(C(N)=O)c(Nc2cn[nH]c2)n1]

In a similar manner as described in Example D-282, 5-[(2R,3R)-3-[(dimethylcarbamoyl)amino]-2-methylpiperidin-1-yl]-3-[(1H-pyrazol-4-yl)amino]pyrazine-2-carboxamide (D-53). MS found for C17H25N9O2 as (M+H)⁺ 432.4.

¹H NMR (400 MHz, DMSO) δ 12.52 (br. s., 1H), 10.85 (s, 1H), 8.07-7.56 (m, 3H), 7.52 (s, 1H), 7.24 (br. s., 1H), 6.09 (d, J=7.02 Hz, 1H), 5.03-4.60 (m, 1H), 4.39-4.09 (m, 1H), 3.77-3.57 (m, 1H), 3.09-2.93 (m, 1H), 2.83 (s, 6H), 1.95-1.45 (m, 4H), 1.09 (d, J=6.80 Hz, 3H).

### Example D-54: Synthesis of (racemic)-cis-5-{1-benzoyl-octahydro-1H-pyrrolo[3,2-b]pyridin-4-yl}-3-[(1-methyl-1H-pyrazol-4-yl)amino]pyrazine-2-carboxamide (D-54)

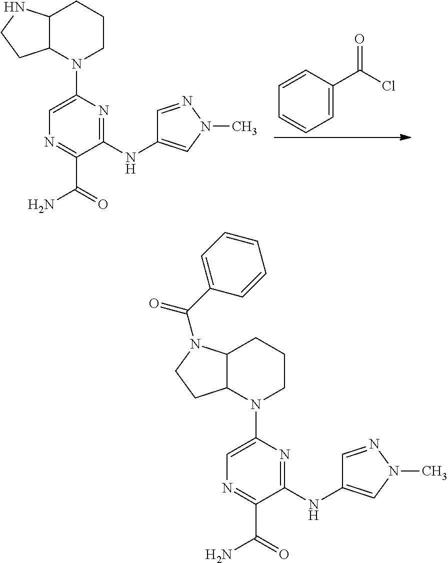

In a similar manner as described in Example 7, (racemic)-cis-5-{1-benzoyl-octahydro-1H-pyrrolo[3,2-b]pyridin-4-yl}-3-[(1-methyl-1H-pyrazol-4-yl)amino]pyrazine-2-carboxamide (D-54) was prepared from 3-[(1-methyl-1H-pyrazol-4-yl)amino]-5-{octahydro-1H-pyrrolo[3,2-b]pyridin-4-yl}pyrazine-2-carboxamide. MS found for C23H26N8O2 as (M+H)⁺ 447.1.

¹H NMR (500 MHz, DMSO) δ 11.06-10.57 (m, 1H), 7.98-7.88 (m, 1H), 7.86-7.81 (m, 1H), 7.70 (br. s., 1H), 7.62 (s, 1H), 7.56-7.51 (m, 1H), 7.50-7.37 (m, 4H), 7.30 (br. s., 1H), 5.05-4.78 (m, 1H), 4.38-3.74 (m, 5H), 3.71-3.36 (m, 2H), 3.07-2.82 (m, 1H), 2.31-1.07 (m, 6H).

### Example D-55: Synthesis of 5-[(2R,3R)-3-(3,3-dimethyl-2,3-dihydro-1-benzofuran-5-amido)-2-methylpiperidin-1-yl]-3-[(1-methyl-1H-pyrazol-4-yl)amino]pyrazine-2-carboxamide (D-55)

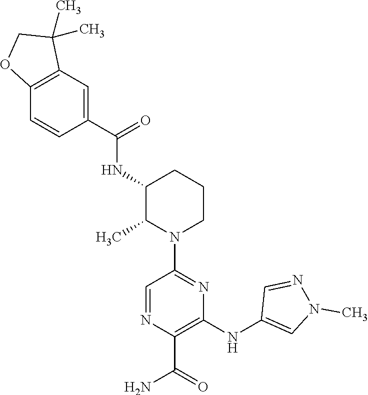 [SMILES: C[C@@H]1[C@H](NC(=O)c2ccc3c(c2)C(C)(C)CO3)CCCN1c1cnc(C(N)=O)c(Nc2cnn(C)c2)n1]

In a similar manner as described in Example D-11, 5-[(2R,3R)-3-(3,3-dimethyl-2,3-dihydro-1-benzofuran-5-amido)-2-methylpiperidin-1-yl]-3-[(I-methyl-1H-pyrazol-4-yl)amino]pyrazine-2-carboxamide (D-55) was prepared. MS found for C26H32N8O3 as (M+H)⁺ 505.2.

¹H NMR (500 MHz, DMSO) δ 10.86 (s, 1H), 8.24 (d, J=6.85 Hz, 1H), 8.03 (br. s., 1H), 7.84-7.76 (m, 2H), 7.69 (br. s., 1H), 7.56 (s, 1H), 7.47 (s, 1H), 7.28 (br. s., 1H), 6.85 (d, J=8.31 Hz, 1H), 5.33 (br. s., 1H), 4.30 (s, 2H), 4.11 (br. s., 1H), 4.03 (m, J=11.98, 11.98, 4.89 Hz, 1H), 4.03 (m, J=11.98, 11.98, 4.89 Hz, 1H), 3.76 (s, 3H), 3.09 (t, J=12.23 Hz, 1H), 1.96 (qd, J=12.96, 3.67 Hz, 1H), 1.96 (qd, J=12.96, 3.67 Hz, 1H), 1.87 (d, J=12.72 Hz, 1H), 1.72 (d, J=9.78 Hz, 1H), 1.67-1.54 (m, 1H), 1.33 (s, 6H), 1.08 (d, J=6.85 Hz, 3H).

### Example D-56: Synthesis of 3-[(1-methyl-1H-pyrazol-4-yl)amino]-5-[(2R,3R)-2-methyl-3-(3-methyl-3,4-dihydro-2H-1-benzopyran-7-amido)piperidin-1-yl]pyrazine-2-carboxamide (D-56)

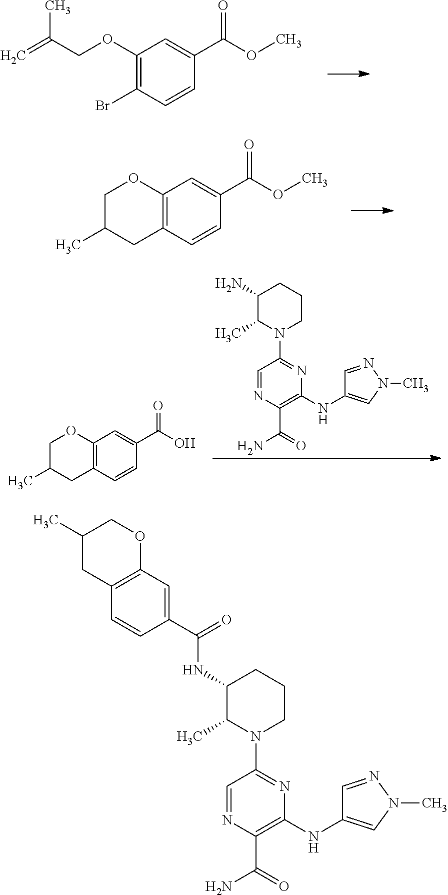

To a solution of methyl 4-bromo-3-[(2-methylprop-2-en-1-yl)oxy]benzoate (289 mg, 1.0 mmol) in 10 mL of dry toluene was added tributhyltin hydride (0.404 mL. 1.50 mmol) and AIBN (6 mg, 0.04 mmol). The mixture was stirred at 110° C. overnight. The mixture was cooled to room temperature and 12 mg of AIBN were added and the reaction was stirred at 120° C. for further 16 hours. The mixture was cooled to room temperature and the toluene was evaporated. The residue was purified by silica flash chromatography with 0% to 50% of ethyl acetate in cyclohexane to give methyl 3-methyl-3,4-dihydro-2H-1-benzopyran-7-carboxylate (17 mg, 8% yield) as a white solid. MS found for C12H1403 as (M+H)⁺ 207.1.

To a solution of methyl 3-methyl-3,4-dihydro-2H-1-benzopyran-7-carboxylate (17 mg, 0.082 mmol) in 1.0 mL of THF and 1.0 mL of MeOH was added at room temperature LiOH.H₂O (7 mg, 0.164 mmol) and 1.0 mL of water. The reaction mixture was stirred at room temperature overnight. The reaction mixture was quenched with aqueous HCl (1N, 5.0 mL) and the product was extracted with ethyl acetate (thee times). The collected organic layers were dried over Na₂SO₄, filtered and evaporated in high vacuum to yield 3-methyl-3,4-dihydro-2H-1-benzopyran-7-carboxylic acid (13 mg, 83% yield) as a white solid. MS found for C11H12O3 as (M+H)⁺ 193.0.

In a similar manner as described in Example D-11, 3-[(1-methyl-1H-pyrazol-4-yl)amino]-5-[(2R,3R)-2-methyl-3-(3-methyl-3,4-dihydro-2H-1-benzopyran-7-amido)piperidin-1-yl]pyrazine-2-carboxamide (D-56) was prepared. MS found for C26H32N8O3 as (M+H)⁺ 505.4.

¹H NMR (500 MHz, DMSO) δ 10.86 (s, 1H), 8.32 (d, J=6.58 Hz, 1H), 8.02 (s, 1H), 7.68 (br. s., 1H), 7.56 (s, 1H), 7.50-7.43 (m, 1H), 7.41-7.36 (m, 1H), 7.33 (s, 1H), 7.29-7.23 (m, 1H), 7.19-7.12 (m, 1H), 5.29 (br. s., 1H), 4.24-3.93 (m, 3H), 3.76 (s, 3H), 3.75-3.66 (m, 1H), 3.07 (t, =12.06 Hz, 1H), 2.86 (dd, J=16.44, 5.04 Hz, 1H), 2.48-2.38 (m, 1H), 2.15-2.01 (m, 1H), 2.03-1.52 (m, 4H), 1.08 (d, J=7.02 Hz, 3H), 1.00 (d, J=6.80 Hz, 3H).

### Example D-57: Synthesis of 5-[(2R,3R)-3-(1-tert-butyl-1H-pyrazole-4-amido)-2-methylpiperidin-1-yl]-3-[(1-methyl-1H-pyrazol-4-yl)amino]pyrazine-2-carboxamide (D-57)

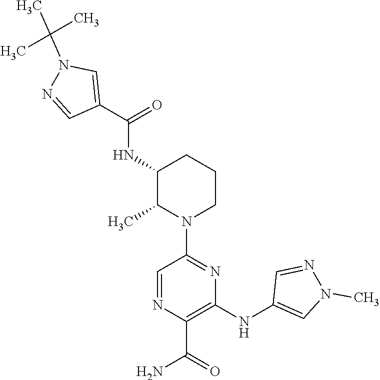 [SMILES: C[C@@H]1[C@H](NC(=O)c2cnn(C(C)(C)C)c2)CCCN1c1cnc(C(N)=O)c(Nc2cnn(C)c2)n1]

In a similar manner as described in Example D-11, 5-[(2R,3R)-3-(1-tert-butyl-1H-pyrazole-4-amido)-2-methylpiperidin-1-yl]-3-[(1-methyl-1H-pyrazol-4-yl)amino]pyrazine-2-carboxamide (D-57) was prepared. MS found for C23H32N10O2 as (M+H)⁺ 481.4.

¹H NMR (500 MHz, DMSO) δ 10.86 (s, 1H), 8.39 (s, 1H), 8.05 (br. s., 1H), 8.00 (d, J=6.86 Hz, 1H), 7.93 (s, 1H), 7.68 (br. s., 1H), 7.55 (s, 1H), 7.45 (s, 1H), 7.27 (br. s., 1H), 5.66-4.98 (m, 1H), 4.24-3.90 (m, 2H), 3.80 (s, 3H), 3.09 (t, J=12.62 Hz, 1H), 1.95-1.58 (m, 4H), 1.57-1.50 (m, 9H), 1.07 (d, J=6.59 Hz, 3H).

### Preparation of 3-{[4-(1-cyclopentyl-4-methylpiperidin-4-yl)phenyl]amino}-5-[3-[(dimethylcarbamoyl)amino]-2-(hydroxymethyl)piperidin-1-yl]pyrazine-2-carboxamide

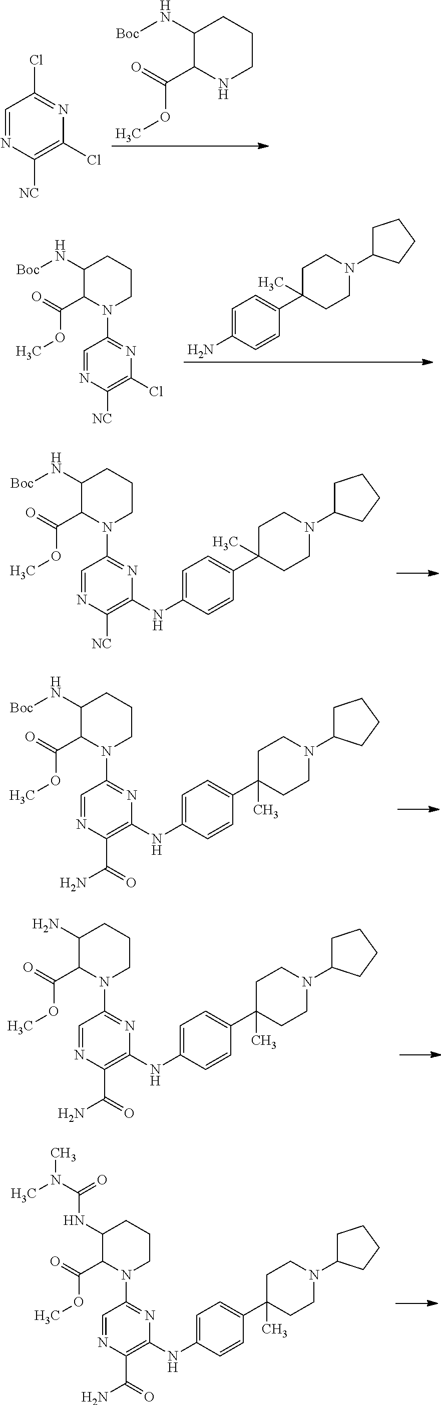

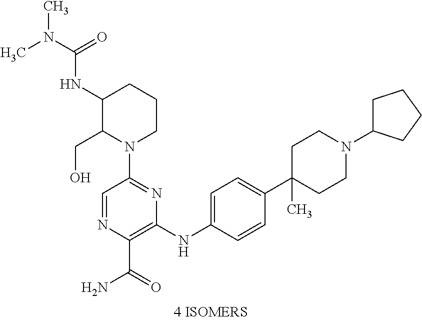 [SMILES: CN(C)C(=O)NC1CCCN(c2cnc(C(N)=O)c(Nc3ccc(C4(C)CCN(C5CCCC5)CC4)cc3)n2)C1CO]

To a solution of 3,5-dichloropyrazine-2-carbonitrile (1.05 g, 6.03 mmol) in DMF (15 mL) was added methyl 3-{[(tert-butoxy)carbonyl]amino}piperidine-2-carboxylate (1.4 g, 5.03 mmol) and DIPEA (1.75 mL, 10.06 mmol). The mixture was stirred at room temperature overnight. The mixture was concentrated in vacuo. The residue was purified by flash chromatography with 30 to 60% ethyl acetate in cyclohexane to give methyl 3-{[(tert-butoxy)carbonyl]amino}-1-(6-chloro-5-cyanopyrazin-2-yl)piperidine-2-carboxylate (1.8 g, 83% yield). MS found for C17H22ClN5O4 as (M+H)⁺ 396.0.

A mixture of 3-{[(tert-butoxy)carbonyl]amino}-1-(6-chloro-5-cyanopyrazin-2-yl)piperidine-2-carboxylate (0.45 g, 1.13 mmol), 4-(1-cyclopentyl-4-methylpiperidin-4-yl)aniline (0.437 g, 1.13 mmol), Pd(OAc)₂ (50.74 mg, 0.226 mmol), (+/−) BINAP (140.7 mg. 0.226 mmol), fine powder Cs₂CO₃ (1.104 g, 3.39 mmol) in dioxane (30 mL) was degassed with a nitrogen stream for 10 min. The mixture was stirred in a nitrogen atmosphere at 115° C. overnight, then cooled to room temperature and concentrated in vacuo. The residue was purified by flash chromatography with 30 to 80% ethyl acetate in cyclohexane to isolate methyl 3-{[(tert-butoxy)carbonyl]amino}-1-(5-cyano-6-{[4-(1-cyclopentyl-4-methylpiperidin-4-yl)phenyl]amino}pyrazin-2-yl)piperidine-2-carboxylate (0.722 g, 78% yield). MS found for C34H47N7O4 as (M+H)⁺ 618.2.

A solution of methyl 3-{[(tert-butoxy)carbonyl]amino}-1-(5-cyano-6-{[4-(1-cyclopentyl-4-methylpiperidin-4-yl}phenyl]amino pyrazin-2-yl)piperidine-2-carboxylate (0.722 g) in TFA (15 mL) and H₂SO₄ (1 mL) was left stirring at room temperature overnight passed through an SCX cartridge and eluted with ammonia in MeOH 7 N. The filtrate was concentrated in vacuo obtaining methyl 3-amino-1-(5-carbamoyl-6-{[4-(1-cyclopentyl-4-methylpiperidin-4-yl)phenyl]amino}pyrazin-2-yl)piperidine-2-carboxylate (522 mg). MS found for C29H41N7O3 as (M+H)⁺ 536.1.

Methyl 3-amino-1-(5-carbamoyl-6-{[4-(1-cyclopentyl-4-methylpiperidin-4-yl)phenyl]amino}pyrazin-2-yl)piperidine-2-carboxylate (522 mg 0.97 mmol) was dissolved in DMF (30 mL). DIPEA (0.334 ml. 1.94 mmol) and N,N-dimethylcarbamoyl chloride (0.098 ml 1.07 mmol) were added and the reaction was left stirring at room temperature overnight. The mixture was concentrated and purified by flash chromatography with 0 to 10% MeOH in DCM to isolate methyl 1-(5-carbamoyl-6-{[4-(1-cyclopentyl-4-methylpiperidin-4-yl)phenyl]amino}pyrazin-2-yl)-3-[(dimethylcarbamoyl)amino]piperidine-2-carboxylate (566 mg 96% yield). MS found for C32H46N8O4 as (M+H)⁺ 607.2.

To a solution of methyl 1-(5-carbamoyl-6-{[4-(1-cyclopentyl-4-methylpiperidin-4-yl)phenyl]amino}pyrazin-2-yl)-3-[(dimethylcarbamoyl)amino]piperidine-2-carboxylate (566 mg, 0.932 mmol) was dissolved in THF (15 ml). To the solution LiAlH₄ 1M solution in THF (1.12 mL) was added dropwise. The mixture was left stirring at room temperature overnight. Further 0.6 mL of LiAlH₄ 1M solution in THF were added and the reaction was stirred 5 hours. Na₂SO₄.10 H₂O was added portionwise, DCM was added and the solid was filtered off. The filtrate was concentrated and purified on by flash chromatography eluting with MeOH in DCM from 2 to 5% obtaining 264 mg as mixture of diasteroisomers. The mixture was purified by preparative chiral HPLC to give:

### Example D-58: 3-{[4-(1-cyclopentyl-4-methylpiperidin-4-yl)phenyl]amino}-5-[(2S,3R)-3-[(dimethylcarbamoyl)amino]-2-(hydroxymethyl)piperidin-1-yl]pyrazine-2-carboxamide (D-58)

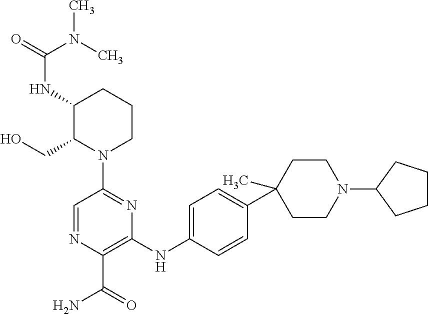 [SMILES: CN(C)C(=O)N[C@@H]1CCCN(c2cnc(C(N)=O)c(Nc3ccc(C4(C)CCN(C5CCCC5)CC4)cc3)n2)[C@@H]1CO]

36.2 mg, 0.062 mmol. MS found for C₃₁H₄₆N₈O₃ as (M+H)⁺ 579.6.

¹H NMR (400 MHz, DMSO) δ 11.26 (s, 1H), 7.72 (br. s., 1H), 7.63 (s, 1H), 7.57 (d, J=8.55 Hz, 2H), 7.28 (d, J=8.55 Hz, 3H), 6.22 (d, J=7.24 Hz, 1H), 4.67 (t, J=5.04 Hz, 1H), 5.11-4.55 (m, 1H), 4.49-4.18 (m, 1H), 3.91-3.66 (m, 3H), 3.08 (t, J=12.39 Hz, 1H), 2.84 (s, 6H), 2.48-2.25 (m, 5H), 2.07-1.18 (m, 16H), 1.14 (s, 3H).

### Example D-59: 3-({[4-(1-cyclopentyl-4-methylpiperidin-4-yl)phenyl]amino}-5-[(2R,3S)-3-[(dimethylcarbamoyl)amino]-2-(hydroxymethyl)piperidin-1-yl]pyrazine-2-carboxamide (D-59)

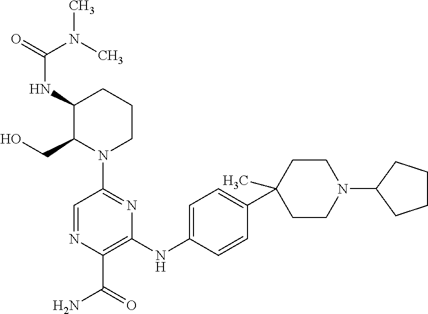 [SMILES: CN(C)C(=O)N[C@H]1CCCN(c2cnc(C(N)=O)c(Nc3ccc(C4(C)CCN(C5CCCC5)CC4)cc3)n2)[C@H]1CO]

32.3 mg, 0.056 mmol; MS found for C31H46N8O3 as (M+H)⁺ 579.6.

¹H NMR (400 MHz, DMSO) δ 11.26 (s, 1H), 7.72 (br. s., 1H), 7.63 (s, 1H), 7.57 (d, J=8.55 Hz, 2H), 7.28 (d, J=8.55 Hz, 3H), 6.22 (d, J=7.24 Hz, 1H), 4.67 (t, J=5.04 Hz, 1H), 5.11-4.55 (m, 1H), 4.49-4.18 (m, 1H), 3.91-3.66 (m, 3H), 3.08 (t, J=12.39 Hz, 1H), 2.84 (s, 6H), 2.48-2.25 (m, 5H), 2.07-1.18 (m, 16H), 1.14 (s, 3H).

### Example D-60: 3-{[4-(1-cyclopentyl-4-methylpiperidin-4-yl)phenyl]amino}-5-[(2R,3R)-3-[(dimethylcarbamoyl)amino]-2-(hydroxymethyl)piperidin-1-yl]pyrazine-2-carboxamide (D-60)

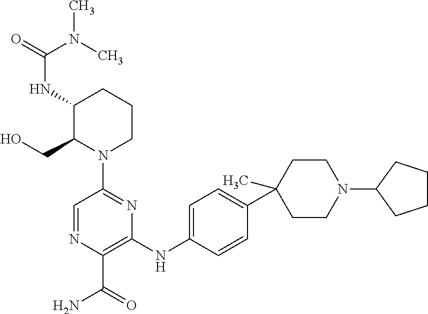 [SMILES: CN(C)C(=O)N[C@@H]1CCCN(c2cnc(C(N)=O)c(Nc3ccc(C4(C)CCN(C5CCCC5)CC4)cc3)n2)[C@H]1CO]

41 mg, 0.07 mmol; MS found for C₃₁H₄₆N₈O₃ as (M+H)⁺ 579.6.

¹H NMR (400 MHz, DMSO) δ 11.23 (s, 1H), 7.74-7.61 (m, 1H), 7.55-7.45 (m, 3H), 7.25-7.16 (m, 3H), 5.78 (d, J=6.80 Hz, 1H), 4.80 (t, J=5.59 Hz, 1H), 4.53-4.41 (m, 1H), 4.19-4.31 (m, 1H), 3.96-3.86 (m, 1H), 3.66-3.53 (m, 2H), 3.10-2.97 (m, 1H), 2.64 (s, 6H), 2.42-2.21 (m, 5H), 2.00-1.15 (m, 16H), 1.08 (s, 3H).

### Example D-61: 3-{[4-(1-cyclopentyl-4-methylpiperidin-4-yl)phenyl]amino}-5-[(2S,3S)-3-[(dimethylcarbamoyl)amino]-2-(hydroxymethyl)piperidin-1-yl]pyrazine-2-carboxamide (D-61)

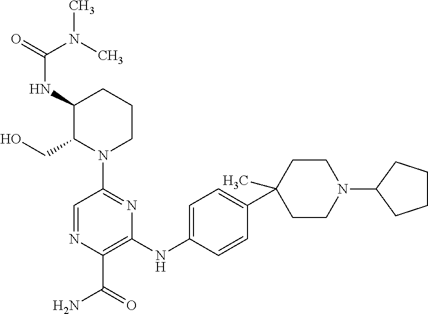 [SMILES: CN(C)C(=O)N[C@H]1CCCN(c2cnc(C(N)=O)c(Nc3ccc(C4(C)CCN(C5CCCC5)CC4)cc3)n2)[C@@H]1CO]

35 mg, 0.06 mmol; MS found for C₃₁H₄₆N₈O₃ as (M+H)⁺ 579.6.

¹H NMR (400 MHz, DMSO) δ 11.23 (s, 1H), 7.74-7.61 (m, 1H), 7.55-7.45 (m, 3H), 7.25-7.16 (m, 3H), 5.78 (d, J=6.80 Hz, 1H), 4.80 (t, J=5.59 Hz, 1H), 4.53-4.41 (m, 1H), 4.19-4.31 (m, 1H), 3.96-3.86 (m, 1H), 3.66-3.53 (m, 2H), 3.10-2.97 (m, 1H), 2.64 (s, 6H), 2.42-2.21 (m, 5H), 2.00-1.15 (m, 16H), 1.08 (s, 3H).

### Example D-62: Synthesis of 5-[(2R,3R)-3-(4-cyclopropyl-2-fluorobenzamido)-2-methylpiperidin-1-yl]-3-({5-methyl-4H,5H,6H,7H-[1,3]thiazolo[5,4-c]pyridin-2-yl}amino)pyrazine-2-carboxamide (D-62)

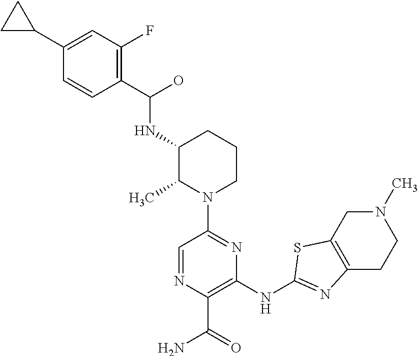 [SMILES: C[C@@H]1[C@H](NC(O)c2ccc(C3CC3)cc2F)CCCN1c1cnc(C(N)=O)c(Nc2nc3c(s2)CN(C)CC3)n1]

In similar manner as described in Example 40, 5-[(2R,3R)-3-(4-cyclopropyl-2-fluorobenzamido)-2-methylpiperidin-1-yl]-3-({5-methyl-4H,5H,6H,7H-[1,3]thiazolo[5,4-c]pyridin-2-yl}amino)pyrazine-2-carboxamide (D-62) was prepared using 5-methyl-4H,5H,6H,7H-1,3]thiazolo[5,4-c]pyridin-2-amine. MS found for C28H33FN8O2S as (M+H)⁺ 565.5

¹H NMR (500 MHz, DMSO) δ 12.38 (br. s., 1H), 8.34 (d, J=7.55 Hz, 1H), 7.91 (d, J=1.65 Hz, 1H), 7.81 (s, 1H), 7.57 (d, J=1.65 Hz, 1H), 7.50-7.40 (m, 1H), 7.07-6.95 (m, 2H), 5.36 (br. s., 1H), 4.53-3.94 (m, 2H), 3.38-3.19 (m, 2H), 3.12 (td, J=13.14, 2.40 Hz, 1H), 2.71-2.56 (m, 4H), 2.27 (br. s., 3H), 2.05-1.96 (m, 1H), 1.94-1.79 (m, 2H), 1.76-1.52 (m, 2H), 1.16 (d, J=6.72 Hz, 3H), 1.05-0.99 (m, 2H), 0.77-0.72 (m, 2H).

### Example D-63: Synthesis of 5-[(2R,3R)-3-(4-cyclopropyl-2-fluorobenzamido)-2-methylpiperidin-1-yl]-3-({5-methyl-4H,5H,6H,7H-[1,3]thiazolo[5,4-c]pyridin-2-yl}amino)pyrazine-2-carboxamide (D-63)

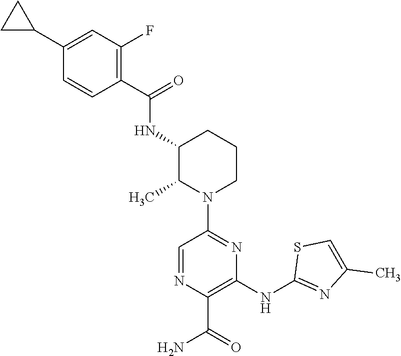 [SMILES: Cc1csc(Nc2nc(N3CCC[C@@H](NC(=O)c4ccc(C5CC5)cc4F)[C@H]3C)cnc2C(N)=O)n1]

In similar manner as described in Example 40, 5-[(2R,3R)-3-(4-cyclopropyl-2-fluorobenzamido)-2-methylpiperidin-1-yl]-3-[(4-methyl-1,3-thiazol-2-yl)amino]pyrazine-2-carboxamide (D-63) was prepared using 4-methyl-1,3-thiazol-2-amine. MS found for C25H28FN7O2S as (M+H)⁺ 510.4.

¹H NMR (500 MHz, DMSO) δ 12.51 (s, 1H), 8.31 (d, J=7.41 Hz, 1H), 7.92 (d, J=1.50 Hz, 1H). 7.83 (s, 1H), 7.59 (d, J=1.51 Hz, 1H), 7.43 (t, J=7.96 Hz, 1H), 7.07-6.94 (m, 2H), 6.63 (s, 1H), 5.17 (br. s., 1H), 4.39 (br. s., 1H), 4.12-3.96 (m, 1H), 3.17-3.03 (m, 1H), 2.24 (s, 3H), 2.04-1.96 (m, 1H), 1.92-1.79 (m, 2H), 1.75-1.56 (m, 2H), 1.18 (d, J=6.86 Hz, 3H), 1.07-0.97 (m, 2H), 0.81-0.70 (m, 2H).

### Example D-64: Synthesis of trans 5-[3-[(dimethylcarbamoyl) amino]-2-(hydroxymethyl) piperidin-1-yl]-3-[(quinolin-6-yl)amino]pyrazine-2-carboxamide (D-64)

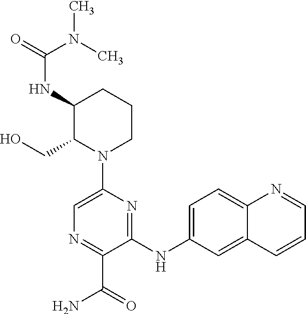 [SMILES: CN(C)C(=O)N[C@H]1CCCN(c2cnc(C(N)=O)c(Nc3ccc4ncccc4c3)n2)[C@@H]1CO]

In similar manner as described in Example D-58, trans-5-{3-[(dimethylcarbamoyl)amino]-2-(hydroxymethyl)piperidin-1-yl}-3-[(quinolin-6-yl)amino]pyrazine-2-carboxamide (D-64) was prepared as racemate using 6-aminoquinoline. MS found for C23H28N8O3 as (M+H)⁺ 465.3.

¹H NMR (400 MHz, DMSO) δ 11.73 (s, 1H), 8.72 (dd, J=4.27, 1.76 Hz, 1H), 8.49 (d, J=2.26 Hz, 1H), 8.26 (d, J=8.28 Hz, 1H), 7.93 (d, J=9.04 Hz, 1H), 7.82 (d, J=2.01 Hz, 1H), 7.75-7.66 (m, 2H), 7.46 (dd, J=8.28, 4.27 Hz, 1H) 7.39 (d, J=2.01 Hz, 1H), 5.89 (d, J=7.03 Hz, 1H), 4.93 (t, J=5.52 Hz, 1H), 4.66 (br. s., 1H), 4.31 (br. s., 1H), 4.13-4.00 (m, 1H), 3.71 (t, J=6.15 Hz, 2H), 3.25-3.14 (m, 1H), 2.66 (s, 6H), 2.08-1.55 (m, 4H).

### Example D-65: Synthesis of cis 5-[3-[(dimethylcarbamoyl) amino]-2-(hydroxymethyl) piperidin-1-yl]-3-[(quinolin-6-yl)amino]pyrazine-2-carboxamide (D-65)

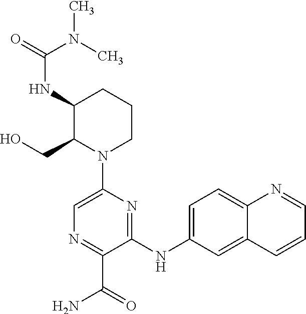 [SMILES: CN(C)C(=O)N[C@H]1CCCN(c2cnc(C(N)=O)c(Nc3ccc4ncccc4c3)n2)[C@H]1CO]

In similar manner as described in Example D-58, cis-5-{3-[(dimethylcarbamoyl)amino]-2-(hydroxymethyl)piperidin-1-yl}-3-[(quinolin-6-yl)amino]pyrazine-2-carboxamide (D-65) was prepared as racemate using 6-aminoquinoline. MS found for C23H28N8O3 as (M+H)⁺ 465.3.

¹H NMR (400 MHz, DMSO) δ 11.71 (s, 1H), 8.72 (dd, J=4.14, 1.63 Hz, 1H), 8.55-8.43 (m, 1H), 8.25 (d, J=7.78 Hz, 1H), 7.94 (d, J=9.03 Hz, 1H), 7.82 (br. s., 1H), 7.76-7.68 (m, 2H), 7.47-7.37 (m, 2H), 6.29 (d, J=7.28 Hz, 1H), 5.32-4.07 (m, 3H), 3.93-3.70 (m, 3H), 3.24-3.08 (m, 1H), 2.85 (s, 6H), 1.96-1.49 (m, 4H).

### Example D-66: Synthesis of 3-{[4-(1-cyclopentyl-4-methylpiperidin-4-yl)phenyl]amino}-5-[(2R,3R)-3-(4-cyclopropyl-2-fluorobenzamido)-2-methylpiperidin-1-yl]pyrazine-2-carboxamide (D-66)

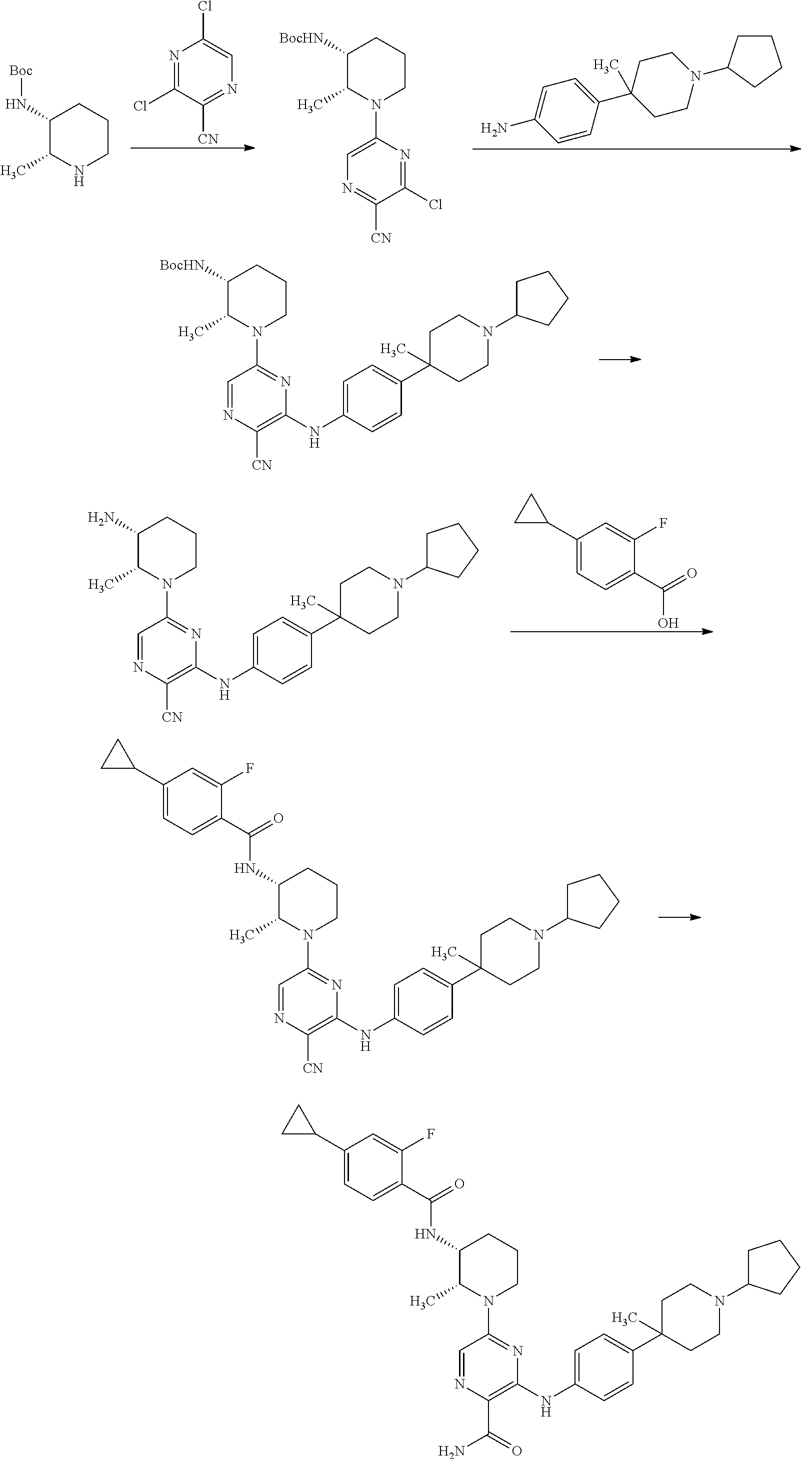

To a solution of 3,5-dichloropyrazine-2-carbonitrile (910 mg, 5.2 mmol) in DMF (15 mL) was added tert-butyl N-[(2R,3R)-2-methylpiperidin-3-yl]carbamate (1.1 g, 1.13 mmol) and DIPEA (1.79 mL, 10.26 mmol). The mixture was stirred at 60° C. for 2 hours. The mixture was concentrated in vacuo. The residue was purified by flash chromatography with 20% ethyl acetate in cyclohexane to isolate tert-butyl N-[(2R,3R)-1-(6-chloro-5-cyanopyrazin-2-yl)-2-methylpiperidin-3-yl]carbamate (1.73 g, 96% yield). MS found for C₁₆H₂₂ClN₅O2 as (M+H)⁺ 352.3.

A mixture of tert-butyl N-[(2R,3R)-1-(6-chloro-5-cyanopyrazin-2-yl)-2-methylpiperidin-3-yl]carbamate (0.2 g, 0.57 mmol), 4-(1-cyclopentyl-4-methylpiperidin-4-yl)aniline (0.220 g, 0.85 mmol), Pd(OAc)₂ (24.7 mg, 0.11 mmol), (+/−) BINAP (68.5 mg, 0.11 mmol), fine powder Cs₂CO₃ (742.9 mg, 2.28 mmol) in dioxane (7 mL) was degassed with a nitrogen stream for 10 min. The mixture was stirred in a nitrogen atmosphere at 90° C. for 4 hours, then cooled to room temperature and concentrated in vacuo. The residue was purified by flash chromatography with 0 to 100% ethyl acetate in cyclohexane to give tert-butyl N-[(2R,3R)-1-(5-cyano-6-{[4-(1-cyclopentyl-4-methylpiperidin-4-yl)phenyl]amino}pyrazin-2-yl)-2-ethylpiperidin-3-yl]carbamate (0.21 g, 64% yield). MS found for C₃₃H4₇N₇O₂ as (M+H)⁺ 572.5.

Tert-butyl N-[(2R,3R)-1-(5-cyano-6-{[4-(1-cyclopentyl-4-methylpiperidin-4-yl) phenyl]amino}pyrazin-2-yl)-2-ethylpiperidin-3-yl]carbamate (0.21 g, 0.37 mmol) was dissolved in HCl in MeOH 1.25M and stirred overnight at room temperature. The solvent was removed and the residue dissolved in MeOH and passed through an SCX cartridge and eluted with ammonia in MeOH 2 N. The filtrate was concentrated in vacuo obtaining 5-[(2R,3R)-3-amino-2-methylpiperidin-1-yl]-3-{[4-(1-cyclopentyl-4-methylpiperidin-4-yl)phenyl]amino}pyrazine-2-carbonitrile (150 mg, 86% yield). MS found for C₂₈H₃₉N₇ as (M+H)⁺ 474.3.

5-[(2R,3R)-3-amino-2-methylpiperidin-1-yl]-3-{[4-(1-cyclopentyl-4-methylpiperidin-4-yl) phenyl]amino}pyrazine-2-carbonitrile (150 mg, 0.32 mmol), 4-cyclopropyl-2-fluorobenzoic acid (69.18 mg, 0.384 mmol) and DIPEA (0.280 ml, 1.6 mmol) were dissolved in DMF (7 ml). PyBOP (199.8 mg. 0.38 mmol) was added and the reaction was left stirring at room temperature overnight. The mixture was concentrated, and taken up with DCM; washed with NaHCO₃ saturated aqueous solution. The layers were separated and the organic one was filtered through a phase separator and concentrated in vacuo. The residue was purified by flash chromatography with 0 to 10% ethyl acetate in cyclohexane to isolate N-[(2R,3R)-1-(5-cyano-6-{[4-(1-cyclopentyl-4-methylpiperidin-4-yl)phenyl]amino}pyrazin-2-yl)-2-methylpiperidin-3-yl]-4-cyclopropyl-2-fluorobenzamide (114 mg, 56% yield). MS found for C₃₈H₄₆FN₇O as (M+H)⁺ 636.2.

N-[(2R,3R)-1-(5-cyano-6-{[4-(1-cyclopentyl-4-methylpiperidin-4-yl)phenyl]amino}pyrazin-2-yl)-2-methylpiperidin-3-yl]-4-cyclopropyl-2-fluorobenzamide (114 mg. 0.179 mmol) was dissolved in MeOH (3 mL) and DMSO (1 mL), TEA (0.5 mL, 3.58 mmol) was added followed by NaOH (18 mg, 0.43 mmol) and H₂O₂ (0.14 ml). The mixture was left stirring at room temperature for 4 hours. The reaction was concentrated in vacuo, DCM and water were added, and the phases were separated. The residue was first filtrated through an SCX cartridge eluting with NH₃ in MeOH 7 N and then purified by flash chromatography eluting with MeOH in DCM from 5 to 10% to give 3-{[4-(1-cyclopentyl-4-methylpiperidin-4-yl)phenyl]amino}-5-[(2R,3R)-3-(4-cyclopropyl-2-fluorobenzamido)-2-methylpiperidin-1-yl]pyrazine-2-carboxamide (D-66) (94 mg, 80% yield). MS found for C38H48FN7O2 as (M+H)⁺ 654.3.

¹H NMR (500 MHz) δ 11.18 (s, 1H), 8.31 (d, J=7.68 Hz, 1H), 7.75 (br. s., 1H), 7.65 (s, 1H), 7.55 (d, J=8.51 Hz, 2H), 7.52-7.45 (m, 1H), 7.33 (br. s., 1H), 7.22 (d, J=8.78 Hz, 2H), 7.05-6.96 (m, 2H), 5.25-5.06 (m, 1H), 4.23-3.97 (m, 2H), 3.07 (t, J=11.94 Hz, 1H), 2.46-2.21 (m, 5H), 2.08-1.95 (m, 1H), 1.73 (br. s., 17H), 1.12 (d, J=6.86 Hz, 3H), 1.06 (s, 3H), 1.06-0.99 (m, 2H), 0.79-0.73 (m, 2H).

### Example D-67: Synthesis of 5-[(2S,3R)-3-[(dimethylcarbamoyl)amino]-2-(hydroxymethyl)piperidin-1-yl]-3-[(3-methyl-1,2-thiazol-5-y) amino]pyrazine-2-carboxamide (D-67)

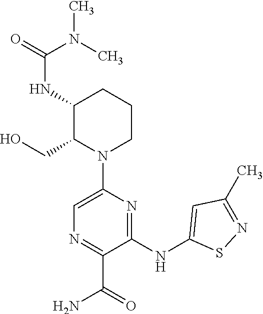 [SMILES: Cc1cc(Nc2nc(N3CCC[C@@H](NC(=O)N(C)C)[C@H]3CO)cnc2C(N)=O)sn1]

In similar manner as described in Example D-58, 5-[(2S,3R)-3-[(dimethylcarbamoyl)amino]-2-(hydroxymethyl)piperidin-1-yl]-3-[(3-methyl-1,2-thiazol-5-yl) amino]pyrazine-2-carboxamide (D-67) was prepared using 5-amino-3-methyl-isothiazole hydrochloride. MS found for C23H28N8O3 as (M+H)⁺ 433.2.

¹H NMR (400 MHz, DMSO) δ 12.15 (s, 1H), 7.84 (br. s., 1H), 7.75 (s, 1H), 7.51-7.41 (m, 1H), 6.80 (s, 1H), 6.22 (d, J=6.80 Hz, 1H), 4.97-4.46 (m, 3H), 3.96-3.60 (m, 3H), 3.14 (t, J=12.28 Hz, 1H), 2.78 (s, 6H), 2.26 (s, 3H), 1.93-1.47 (m, 4H).

### Example D-68: Synthesis of 5-[(2R,3S)-3-[(dimethylcarbamoyl)amino]-2-(hydroxymethyl)piperidin-1-yl]-3-[(3-methyl-1,2-thiazol-5-yl)amino]pyrazine-2-carboxamide (D-68)

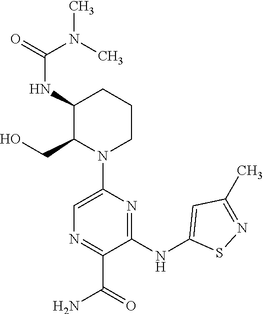 [SMILES: Cc1cc(Nc2nc(N3CCC[C@H](NC(=O)N(C)C)[C@@H]3CO)cnc2C(N)=O)sn1]

In similar manner as described in Example D-58, 5-[(2R,3S)-3-[(dimethylcarbamoyl)amino]-2-(hydroxymethyl)piperidin-1-yl]-3-[(3-methyl-1,2-thiazol-5-yl) amino]pyrazine-2-carboxamide (D-68) was prepared using 5-amino-3-methyl-isothiazole hydrochloride. MS found for C23H28N8O3 as (M+H)⁺ 433.2.

¹H NMR (400 MHz, DMSO) δ 12.15 (s, 1H), 7.84 (br. s., 1H), 7.75 (s, 1H), 7.51-7.41 (m, 1H), 6.80 (s, 1H), 6.22 (d, J=6.80 Hz, 1H), 4.97-4.46 (m, 3H), 3.96-3.60 (m, 3H), 3.14 (t, J=12.28 Hz, 1H), 2.78 (s, 6H), 2.26 (s, 3H), 1.93-1.47 (m, 4H).

### Example D-69: Synthesis of 3-{[4-(4-methylpiperazine-1-carbonyl)phenyl]amino}-5-{[(1r,4r)-4-[(dimethylcarbamoyl) amino]cyclohexyl] amino} pyrazine-2-carboxamide (D-69)

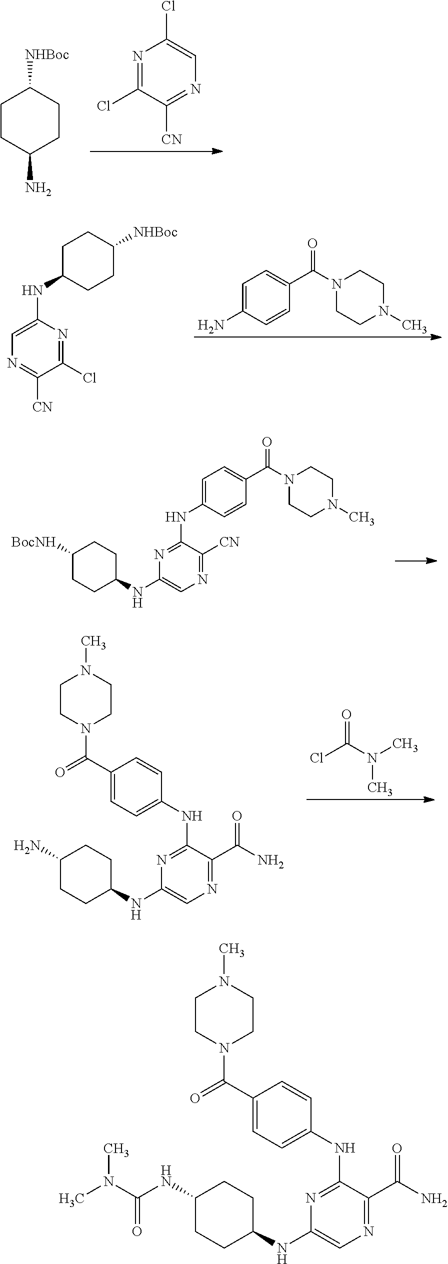

To a solution of 3,5-dichloropyrazine-2-carbonitrile (1.48 g, 8.48 mmol) in DMF (40 mL) was added tert-butyl N-[(1r,4r)-4-aminocyclohexyl]carbamate (2 g, 9.33 mmol) and DIPEA (2.95 mL, 16.96 mmol). The mixture was stirred at room temperature for 4 hours. The mixture was concentrated in vacuo. DCM and water were added and the phases were separated. The organics were washed with brine, filtered through a phase separator and concentrated. The residue triturated with Et₂O and dried in vacuo to give tert-butyl N-[(1r,4r)-4-[(6-chloro-5-cyanopyrazin-2-yl)amino]cyclohexyl]carbamate (2.28 g, 76.4% yield). MS found for C16H22ClN5O2 as (M+H)⁺ 352.05.

A mixture of tert-butyl tert-butyl N-[(1r,4r)-4-[(6-chloro-5-cyanopyrazin-2-yl)amino]cyclohexyl]carbamate (0.3 g, 0.89 mmol), methylpiperazine-1-carbonyl)aniline (0.39 g, 1.77 mmol), Pd(OAc)₂ (40 mg, 0.177 mmol), (+/−) BINAP (110 mg, 0.177 mmol), fine powder Cs₂CO₃ (0.87 g, 2.67 mmol) in dioxane (50 mL) was degassed with a nitrogen stream for 10 min. The mixture was stirred in a nitrogen atmosphere at 110° C. overnight, then cooled to room temperature and concentrated in vacuo. The residue was purified by flash chromatography with 20 to 100% ethyl acetate in cyclohexane to give tert-butyl tert-butyl N-[(1r,4r)-4-[(5-cyano-6-{[4-(4-methylpiperazine-1-carbonyl)phenyl]amino}pyrazin-2-yl)amino]cyclohexyl]carbamate (0.289 g, 61% yield). MS found for C28H38N8O3 as (M+H)⁺ 535.18.

A solution of tert-butyl N-[(1r,4r)-4-[(5-cyano-6-{[4-(4-methylpiperazine-1-carbonyl)phenyl]amino}pyrazin-2-yl)amino]cyclohexyl]carbamate (0.289 g, 0.54 mmol) in TFA (3 mL) and H₂SO₄ (0.1 mL) was left stirring at room temperature overnight quenched with a saturated aqueous solution of NaHCO₃, concentrated and passed through an SCX cartridge eluting with ammonia in MeOH 7 N. The filtrate was concentrated in vacuo obtaining 3-{[4-(4-methylpiperazine-1-carbonyl)phenyl]amino}-5-{[(1r,4r)-4-aminocyclohexyl] amino} pyrazine-2-carboxamide (200 mg, 81.8%). MS found for C₂₃H₃₂N₈O₂ as (M+H)⁺ 453.15. 3-{[4-(4-methylpiperazine-1-carbonyl)phenyl]amino}-5-{[(1r,4r)-4-aminocyclohexyl] amino} pyrazine-2-carboxamide (84 mg 0.185 mmol) was dissolved in DMF (3 ml). DIPEA (0.097 ml, 0.555 mmol) and N,N-dimethylcarbamoyl chloride (0.02 ml 0.222 mmol) were added and the reaction was left stirring at room temperature overnight. The mixture was concentrated and purified by flash chromatography with 5 to 20% MeOH in DCM to isolate 3-{[4-(4-methylpiperazine-1-carbonyl)phenyl]amino}-5-{[(1r,4r)-4-[(dimethylcarbamoyl) amino] cyclohexyl] amino}pyrazine-2-carboxamide (D-69) (49 mg 50.6%0/yield). MS found for C26H37N9O3 as (M+H)⁺ 524.3.

¹H NMR (500 MHz, DMSO) δ 11.65 (s, 1H), 7.85-7.66 (m, 4H), 7.42-7.33 (m, 3H), 7.32-7.27 (m, 1H), 6.00 (d, J=7.96 Hz, 1H), 3.69-3.40 (m, 6H), 2.78 (s, 6H), 2.46-2.26 (m, 4H), 2.25-2.16 (m, 3H), 2.07 (d, J=10.98 Hz, 2H), 1.85 (d, J=11.25 Hz, 2H), 1.47-1.36 (m, 2H), 1.35-1.27 (m, 2H).

### Example D-70: Synthesis of 3-{[4-(4-methylpiperazine-1-carbonyl)phenyl]amino}-5-{[(1r,4r)-4-(4-cyclopropyl-2-fluorobenzamido)cyclohexyl]amino}pyrazine-2-carboxamide (D-70)

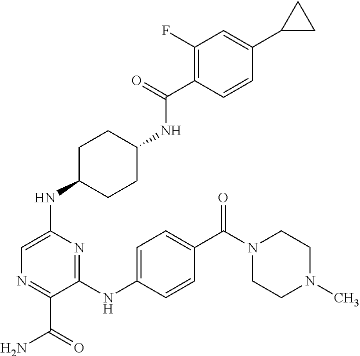 [SMILES: CN1CCN(C(=O)c2ccc(Nc3nc(N[C@H]4CC[C@H](NC(=O)c5ccc(C6CC6)cc5F)CC4)cnc3C(N)=O)cc2)CC1]

In a similar manner as described in Example D-69, 3-{[4-(4-methylpiperazine-1-carbonyl)phenyl]amino}-5-{[(1r,4r)-4-(4-cyclopropyl-2-fluorobenzamido) cyclohexyl]amino}pyrazine-2-carboxamide (D-70) was prepared using 4-cyclopropyl-2-fluorobenzoic acid. MS found for C33H39FN8O3 as (M+H)+ 615.3.

¹H NMR (400 MHz, DMSO) δ 11.64 (s, 1H), 8.09 (d, J=6.14 Hz, 1H), 7.89-7.65 (m, 4H). 7.46 (t, J=7.89 Hz, 1H), 7.41-7.25 (m, 4H), 7.02-6.91 (m, 2H), 3.90-3.76 (m, 1H), 3.71 (br. s., 1H), 3.48 (br. s., 4H), 2.37-2.21 (m, 4H), 2.18-2.04 (m, 5H), 2.04-1.89 (m, 3H), 1.58-1.29 (m, 4H), 1.07-0.96 (m, 2H), 0.78-0.70 (m, 2H).

### Example D-71: Synthesis of 3-{[4-(4-methylpiperazine-1-carbonyl)phenyl]amino}-5-({[(1s,4s)-4-(4-cyclopropyl-2-fluorobenzamido)cyclohexyl]amino}pyrazine-2-carboxamide (D-71)

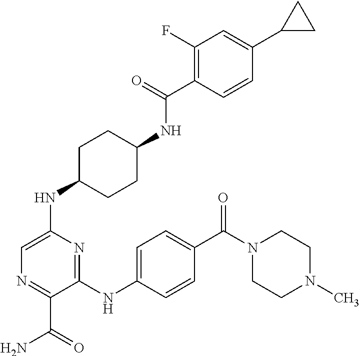 [SMILES: CN1CCN(C(=O)c2ccc(Nc3nc(N[C@H]4CC[C@@H](NC(=O)c5ccc(C6CC6)cc5F)CC4)cnc3C(N)=O)cc2)CC1]

In a similar manner as described in Example D-69, 3-{[4-(4-methylpiperazine-1-carbonyl)phenyl]amino}-5-{[(s,4s)-4-(4-cyclopropyl-2-fluorobenzamido) cyclohexyl] amino}pyrazine-2-carboxamide (D-71) was prepared using 4-cyclopropyl-2-fluorobenzoic acid. MS found for C33H39FN8O3 as (M+H)⁺ 615.2.

¹H NMR (400 MHz, DMSO) δ 11.70-11.54 (m, 1H), 8.05-7.96 (m, 1H), 7.78-7.65 (m, 4H), 7.50-7.42 (m, 2H), 7.35 (d, J=8.77 Hz, 2H), 7.32-7.26 (m, 1H), 7.02-6.93 (m, 2H), 4.04-3.82 (m, 2H), 3.61-3.40 (m, 4H), 2.39-2.26 (m, 4H), 2.20 (s, 3H), 2.05-1.94 (m, 1H), 1.89-1.64 (m, 8H), 1.05-0.97 (m, 2H), 0.80-0.70 (m, 2H).

### Example D-72: Synthesis of 3-{[4-(4-methylpiperazine-1-carbonyl)phenyl]amino}-5-{[(1s,4s)-4-[(dimethylcarbamoyl)amino]cyclohexyl]amino}pyrazine-2-carboxamide (D-72)

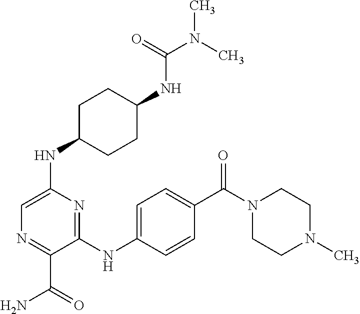 [SMILES: CN1CCN(C(=O)c2ccc(Nc3nc(N[C@H]4CC[C@@H](NC(=O)N(C)C)CC4)cnc3C(N)=O)cc2)CC1]

In a similar manner as described in Example D-69, 3-{[4-(4-methylpiperazine-1-carbonyl)phenyl]amino}-5-{[(1s,4s)-4-[(dimethylcarbamoyl) amino] cyclohexyl] amino} pyrazine-2-carboxamide was prepared. MS found for C26H37N9O3 as (M+H)⁺ 568.3

¹H NMR (400 MHz, DMSO) δ 11.76-11.48 (m, 1H), 7.77-7.69 (m, 3H), 7.68-7.56 (m, 1H), 7.46 (s, 1H), 7.34 (d, J=8.55 Hz, 2H), 7.32-7.27 (m, 1H), 5.76 (d, J=6.36 Hz, 1H), 3.95-3.83 (m, 1H), 3.65-3.42 (m, 5H), 2.79 (s, 6H), 2.39-2.25 (m, 4H), 2.19 (s, 3H), 1.88-1.53 (m, 8H).

### Example D-73: Synthesis of 5-[(2R,3R)-3-(4-cyclopropyl-2-fluorobenzamido)-2-methylpiperidin-1-yl]-3-{[4-(morpholin-4-yl)phenyl]amino}pyrazine-2-carboxamide (D-73)

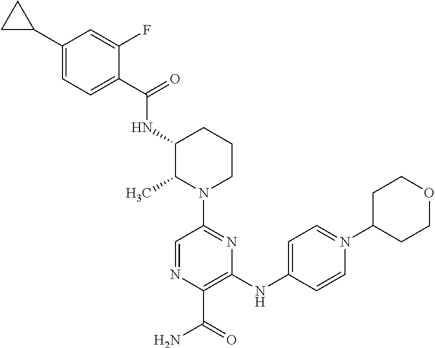

In similar manner as described in Example 40, 5-[(2R,3R)-3-(4-cyclopropyl-2-fluorobenzamido)-2-methylpiperidin-1-yl]-3-{[4-(morpholin-4-yl)phenyl]amino}pyrazine-2-carboxamide (D-73) was prepared using 4-(morpholin-4-yl)aniline. MS found for C31H36FN7O3 as (M+H)⁺ 574.2.

¹H NMR (400 MHz, DMSO) δ 10.97 (s, 1H), 8.30 (d, J=7.45 Hz, 1H), 7.71 (br. s., 1H), 7.60 (s, 1H), 7.53-7.41 (m, 3H), 7.32-7.24 (m, 1H), 7.04-6.96 (m, 2H), 6.83 (d, J=8.99 Hz, 2H), 5.23-4.97 (m, 1H), 4.22-3.98 (m, 2H), 3.68-3.62 (m, 4H), 3.04 (t, J=12.17 Hz, 1H), 2.95-2.83 (m, 4H), 2.09-1.94 (m, 1H), 1.91-1.49 (m, 4H), 1.10 (d, J=7.02 Hz, 3H), 1.07-0.99 (m, 2H), 0.80-0.72 (m, 2H).

### Preparation of 5-{5-[(dimethylcarbamoyl)amino]-2-methylpiperidin-1-yl}-3-[(3-methyl-1,2-thiazol-5-yl)amino]pyrazine-2-carboxamide

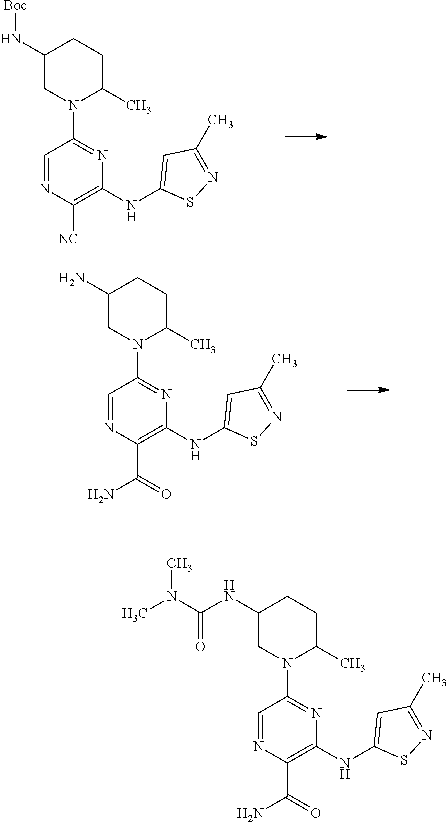

In a similar manner as described in Example D-181, tert-butyl N-(1-{5-cyano-6-[(3-methyl-1,2-thiazol-5-yl)amino]pyrazin-2-yl}-6-methylpiperidin-3-yl)carbamate was prepared as diastereoisomeric mixture. MS found for C20H27N7O2S as (M+H)⁺ 430.1.

A mixture of tert-butyl N-(1-{5-cyano-6-[(3-methyl-1,2-thiazol-5-yl)amino]pyrazin-2-yl}-6-methylpiperidin-3-yl)carbamate (600 mg, 1.39 mmol) in TFA (12 mL) and H₂SO₄ (1 mL) was left stirring at room temperature overnight. To the mixture was added carefully NaHCO₃ saturated solution in water. The mixture was concentrated and passed through an SCX cartridge eluting with NH₃ 7N solution in MeOH. The solvent was removed to give 5-(5-amino-2-methylpiperidin-1-yl)-3-[(3-methyl-1,2-thiazol-5-yl)amino]pyrazine-2-carboxamide (150 mg, 0.43 mmol) as distereoisomeric mixture. MS found for C15H21N7OS as (M+H)⁺ 348.1.

5-(5-amino-2-methylpiperidin-1-yl)-3-[(3-methyl-1,2-thiazol-5-yl)amino]pyrazine-2-carboxamide (150 mg, 0.43 mmol), N,N-dimethylcarbomoylchloride (0.05 mL, 0.51 mmol) and DIPEA (0.224 ml, 1.29 mmol) in DMF (6 ml) were stirred at room temperature for 5 h, then the mixture was concentrated and purified The combined organic phases were concentrated and purified by flash chromatography eluting with MeOH in DCM from 5 to 10% obtaining 97 mg as mixture of diasteroisomers. The mixture was purified by preparative chiral HPLC to give:

### Example D-74: 5-[(2S,5R)-5-[(dimethylcarbamoyl)amino]-2-methylpiperidin-1-yl]-3-[(3-methyl-1,2-thiazol-5-yl)amino]pyrazine-2-carboxamide (D-74)

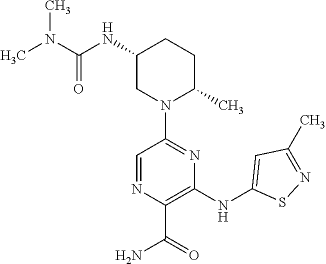

25 mg, 0.06 mmol. MS found for C18H26N8O2S as (M+H)⁺ 419.1.

¹H NMR (500 MHz, CDCl₃) δ 11.84 (s, 1H), 7.70 (s, 1H), 7.43 (br. s., 1H), 6.62 (s, 1H), 5.36 (br. s., 1H), 5.21 (br. s., 1H), 4.49 (dd, J=12.76, 3.84 Hz, 1H), 4.33 (d, J=6.31 Hz, 1H), 3.82-3.70 (m, 1H), 2.96 (s, 6H), 2.89 (t, J=12.80 Hz, 1H), 2.42 (s, 3H), 2.01-1.89 (m, 2H), 1.87-1.60 (m, 2H), 1.35 (d, J=7.00 Hz, 3H).

### Example D-75: 5-[(2R,5S)-5-[(dimethylcarbamoyl)amino]-2-methylpiperidin-1-yl]-3-[(3-methyl-1,2-thiazol-5-yl)amino]pyrazine-2-carboxamide (D-75)

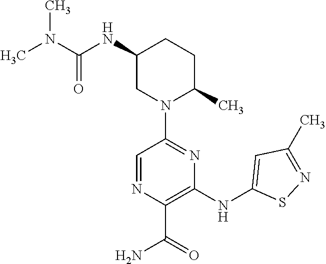

23 mg, 0.055. MS found for C18H26N8O2S as (M+H)⁺ 419.1.

¹H NMR (500 MHz, CDCl₃) δ 11.84 (s, 1H), 7.70 (s, 1H), 7.43 (br. s., 1H), 6.62 (s, 1H), 5.36 (br. s., 1H), 5.21 (br. s., 1H), 4.49 (dd, J=12.76, 3.84 Hz, 1H), 4.33 (d, J=6.31 Hz, 1H), 3.82-3.70 (m, 1H), 2.96 (s, 6H), 2.89 (t, J=12.80 Hz, 1H), 2.42 (s, 3H), 2.01-1.89 (m, 2H), 1.87-1.60 (m, 2H), 1.35 (d, J=7.00 Hz, 3H).

### Example D-76, PCI-58303: 5-[(2S,5S)-5-[(dimethylcarbamoyl)amino]-2-methylpiperidin-1-yl]-3-[(3-methyl-1,2-thiazol-5-yl)amino]pyrazine-2-carboxamide (D-76)

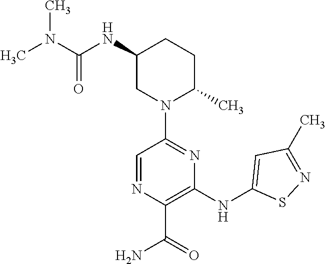

5 mg, 0.012 mmol. MS found for C18H26N8O2S as (M+H)⁺ 419.1.

¹H NMR (500 MHz, CDCl₃) δ 11.91 (br. s., 1H), 7.56 (s, 1H), 7.42 (br. s., 1H), 6.65 (s, 1H), 5.31 (br. s., 1H), 5.03 (br. s., 1H), 4.65 (d, J=6.17 Hz, 1H), 4.44 (d, J=14.00 Hz, 1H), 4.22-4.13 (m, 1H), 3.42 (dd, J=14.00, 2.74 Hz, 1H), 2.81 (s, 6H), 2.44 (s, 3H), 2.17-1.96 (m, 2H), 1.86-1.75 (m, 1H), 1.72-1.55 (m, 1H), 1.37 (d, J=6.72 Hz, 3H).

### Example D-77: 5-[(2R,5R)-5-[(dimethylcarbamoyl)amino]-2-methylpiperidin-1-yl]-3-[(3-methyl-1,2-thiazol-5-yl)amino]pyrazine-2-carboxamide (D-77)

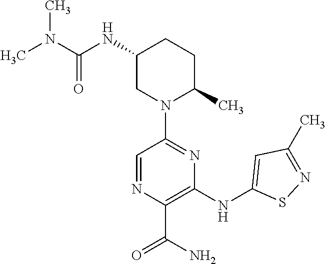

4 mg, 0.009 mmol. MS found for C18H26N8O2S as (M+H)⁺ 419.1.

¹H NMR (500 MHz, CDCl₃) δ 11.91 (br. s., 1H), 7.56 (s, 1H), 7.42 (br. s., 1H), 6.65 (s, 1H), 5.31 (br. s., 1H), 5.03 (br. s., 1H), 4.65 (d, J=6.17 Hz, 1H), 4.44 (d, J=14.00 Hz, 1H), 4.22-4.13 (m, 1H), 3.42 (dd, J=14.00, 2.74 Hz, 1H), 2.81 (s, 6H), 2.44 (s, 3H), 2.17-1.96 (m, 2H), 1.86-1.75 (m, 1H), 1.72-1.55 (m, 1H), 1.37 (d, J=6.72 Hz, 3H).

### Preparation of 5-[5-(4-cyclopropyl-2-fluorobenzamido)-2-methylpiperidin-1-yl]-3-[(3-methyl-1,2-thiazol-5-yl)amino]pyrazine-2-carboxamide

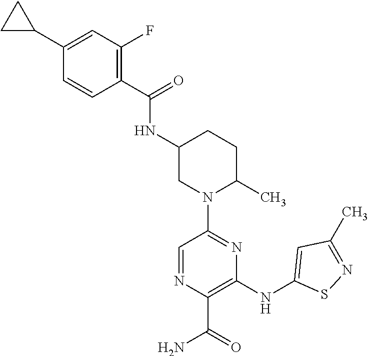 [SMILES: Cc1cc(Nc2nc(N3CC(NC(=O)c4ccc(C5CC5)cc4F)CCC3C)cnc2C(N)=O)sn1]

5-[5-(4-cyclopropyl-2-fluorobenzamido)-2-methylpiperidin-1-yl]-3-[(3-methyl-1,2-thiazol-5-yl)amino]pyrazine-2-carboxamide was obtained in a similar manner as described for D-181 as distereoisomeric mixture. MS found for C25H28FN7O2S as (M+H)⁺ 510.1. The mixture was purified by preparative chiral HPLC to give:

### Example D-78: 5-[(2S,5S)-5-(4-cyclopropyl-2-fluorobenzamido)-2-methylpiperidin-1-yl]-3-[(3-methyl-1,2-thiazol-5-yl)amino]pyrazine-2-carboxamide (D-78)

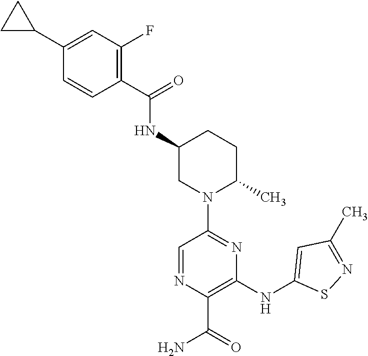 [SMILES: Cc1cc(Nc2nc(N3C[C@@H](NC(=O)c4ccc(C5CC5)cc4F)CC[C@@H]3C)cnc2C(N)=O)sn1]

21 mg, 0.041 mmol. MS found for C25H28FN7O2S as (M+H)⁺ 510.1.

¹H NMR (500 MHz, CDCl₃) δ 11.97 (br. s., 1H), 7.93 (t, J=8.30 Hz, 1H), 7.57 (s, 1H), 7.39 (br. s., 1H), 7.02-6.89 (m, 2H), 6.72-6.59 (m, 2H), 5.29 (br. s., 1H), 5.12 (br. s., 1H), 4.59-4.45 (m, 2H), 3.50 (dd, J=14.13, 2.33 Hz, 1H), 2.46 (s, 3H), 2.22-2.02 (m, 2H), 1.98-1.83 (m, 2H), 1.75-1.67 (m, 1H), 1.41 (d, J=6.86 Hz, 3H), 1.13-0.98 (m, 2H), 0.78-0.66 (m, 2H).

### Example D-79: 5-[(2R,5R)-5-(4-cyclopropyl-2-fluorobenzamido)-2-methylpiperidin-1-yl]-3-[(3-methyl-1,2-thiazol-5-yl)amino]pyrazine-2-carboxamide (D-79)

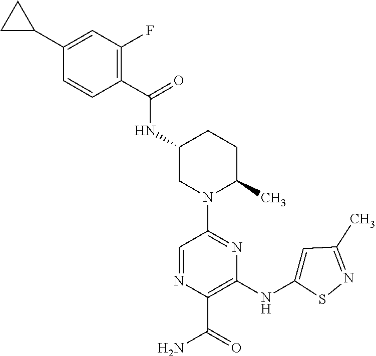 [SMILES: Cc1cc(Nc2nc(N3C[C@H](NC(=O)c4ccc(C5CC5)cc4F)CC[C@H]3C)cnc2C(N)=O)sn1]

20 mg, 0.039 mmol. MS found for C25H28FN7O2S as (M+H)⁺ 510.1.

₁H NMR (500 MHz, CDCl₃) δ 11.97 (br. s., 1H), 7.93 (t, J=8.30 Hz, 1H), 7.57 (s, 1H), 7.39 (br. s., 1H), 7.02-6.89 (m, 2H), 6.72-6.59 (m, 2H), 5.29 (br. s., 1H), 5.12 (br. s., 1H), 4.59-4.45 (m, 2H), 3.50 (dd, J=14.13, 2.33 Hz, 1H), 2.46 (s, 3H), 2.22-2.02 (m, 2H), 1.98-1.83 (m, 2H), 1.75-1.67 (m, 1H), 1.41 (d, J=6.86 Hz, 3H), 1.13-0.98 (m, 2H), 0.78-0.66 (m, 2H).

### Example D-80: 5-[(2R5S)-5-(4-cyclopropyl-2-fluorobenzamido)-2-methylpiperidin-1-yl]-3-[(3-methyl-1,2-thiazol-5-yl)amino]pyrazine-2-carboxamide (D-80)

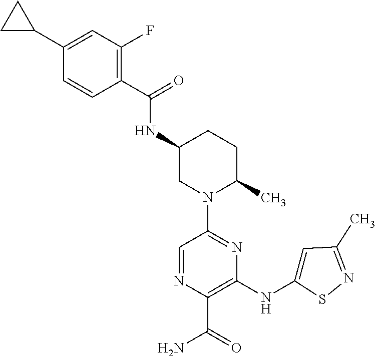 [SMILES: Cc1cc(Nc2nc(N3C[C@@H](NC(=O)c4ccc(C5CC5)cc4F)CC[C@H]3C)cnc2C(N)=O)sn1]

20 mg, 0.05 mmol. MS found for C25H28FN7O2S as (M+H)⁺ 510.1.

¹H NMR (500 MHz, CDCl₃) δ 11.94 (br. s., 1H), 8.01 (t. J=8.37 Hz, 1H), 7.73 (s, 1H), 7.45 (br. s., 1H), 6.99 (dd, J=8.23, 1.51 Hz, 1H), 6.81 (dd, J=13.79, 1.44 Hz, 1H), 6.73-6.59 (m, 2H), 5.34 (br. s., 1H), 5.22 (br. s., 1H), 4.57 (dd, J=12.90, 3.70 Hz, 1H), 4.19-4.05 (m, 1H), 3.03 (dd, J=12.90, 11.25 Hz, 1H), 2.45 (s, 3H), 2.14-1.91 (m, 3H), 1.89-1.77 (m, 2H), 1.39 (d, J=6.86 Hz, 3H), 1.13-1.06 (m, 2H), 0.81-0.75 (m, 2H).

### Example D-81: Synthesis of 5-[(2R,3R)-3-(4-cyclopropylbenzamido)-2-methylpiperidin-1-yl]-3-{[4-(4-methylpiperazin-1-yl)phenyl]amino}pyrazine-2-carboxamide (D-81)

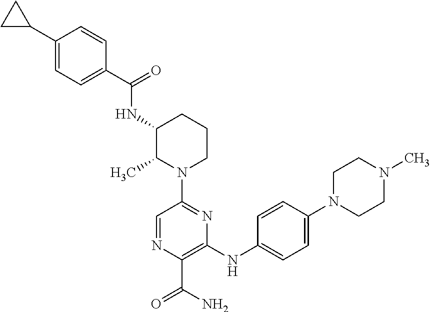 [SMILES: C[C@@H]1[C@H](NC(=O)c2ccc(C3CC3)cc2)CCCN1c1cnc(C(N)=O)c(Nc2ccc(N3CCN(C)CC3)cc2)n1]

In a similar manner as described in Example 8, 5-[(2R,3R)-3-(4-cyclopropylbenzamido)-methylpiperidin-1-yl]-3-{[4-(4methylpiperazin-1-yl)phenyl]amino}pyrazine-2-carboxamide (D-81) was prepared using 4-methylpiperazin-1-yl)aniline MS found for C₃₂H₄₀N₈O₂ as (M+H)⁺ 569.4.

¹H NMR (500 MHz, DMSO) δ 10.93 (br. s., 1H), 8.34 (d, J=7.68 Hz, 1H), 7.84 (d, J=8.23 Hz, 2H), 7.70 (br. s., 1H), 7.59 (s, 1H), 7.44 (d, J=9.06 Hz, 2H), 7.27 (d, J=1.92 Hz, 1H), 7.18 (d, J=8.23 Hz, 2H), 6.78 (d, J=8.23 Hz, 2H), 5.14 (br. s., 1H), 4.34-3.97 (m, 2H), 3.06 (t, J=12.35 Hz, 1H), 2.98-2.77 (m, 4H), 2.36 (d, J=2.47 Hz, 4H), 2.20 (s, 3H), 1.97-2.05 (m, 1H), 1.96-1.89 (m, 1H), 1.84 (d, J=13.17 Hz, 1H), 1.74-1.52 (m, 2H), 1.11-1.00 (m, 5H), 0.79-0.72 (m, 2H).

### Example D-82: Synthesis of 5-[(2R,3R)-3-(4-cyclopropyl-2-fluorobenzamido)-2-methylpiperidin-1-yl]-3-{[3-fluoro-4-(4-methylpiperazin-1-yl)phenyl]amino}pyrazine-2-carboxamide (D-82)

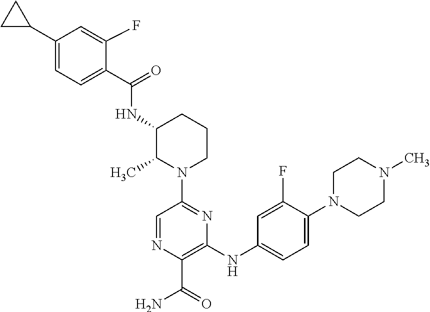 [SMILES: C[C@@H]1[C@H](NC(=O)c2ccc(C3CC3)cc2F)CCCN1c1cnc(C(N)=O)c(Nc2ccc(N3CCN(C)CC3)c(F)c2)n1]

In a similar manner as described in Example 8, 5-[(2R,3R)-3-(4-cyclopropyl-2-fluorobenzamido)-2-methylpiperidin-1-yl]-3-{[3-fluoro-4-(4-methylpiperazin-1-yl)phenyl]amino}pyrazine-2-carboxamide (D-82) was prepared using 4-methylpiperazin-1-yl)aniline MS found for C32H38F2N8O2 as (M+H)⁺ 605.6.

¹H NMR (500 MHz, DMSO) δ 11.17 (s, 1H), 8.30 (d, J=7.55 Hz, 1H), 7.76 (br. s., 1H), 7.66 (s, 1H), 7.53-7.41 (m, 2H), 7.35 (br. s., 1H), 7.26 (d, J=8.37 Hz, 1H), 7.05-6.96 (m, 2H), 6.90 (t, J=9.33 Hz, 1H), 5.03 (br. s., 1H), 4.13 (br. s., 1H), 4.08-3.97 (m, 1H), 3.11-2.97 (m, 1H), 2.84 (br. s., 4H), 2.38 (br. s., 4H), 2.20 (s, 3H), 2.05-1.96 (m, 1H), 1.89-1.78 (m, 2H), 1.69-1.53 (m, 2H), 1.15 (d, J=6.86 Hz, 3H), 1.07-0.99 (m, 2H), 0.81-0.72 (m, 2H).

### Example D-83: Synthesis of 5-[(2R,3R)-3-(4-cyclopropylbenzamido)-2-methylpiperidin-1-yl]-3-({imidazo[1,2-a]pyridin-6-yl}amino)pyrazine-2-carboxamide (D-83)

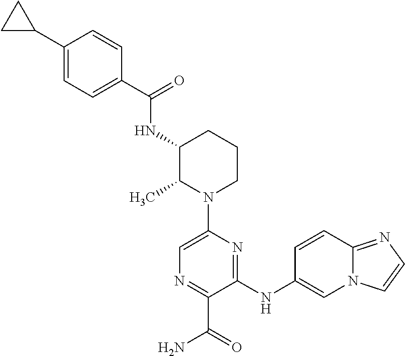 [SMILES: C[C@@H]1[C@H](NC(=O)c2ccc(C3CC3)cc2)CCCN1c1cnc(C(N)=O)c(Nc2ccc3nccn3c2)n1]

In a similar manner as described in Example 8, 5-[(2R,3R)-3-(4-cyclopropylbenzamido)-2-methylpiperidin-1-yl]-3-({imidazo[1,2-a]pyridin-6-yl}amino)pyrazine-2-carboxamide (D-83) was prepared. MS found for C28H30N8O2 as (M+H)⁺ 511.3.

¹H NMR (500 MHz, DMSO) δ 11.54-11.28 (m, 1H), 9.15 (br. s., 1H), 8.42 (d, J=6.86 Hz, 1H), 7.91 (s, 1H), 7.88-7.80 (m, 3H), 7.71 (s, 1H), 7.49 (d, J=9.33 Hz, 1H), 7.44 (br. s., 1H), 7.37 (br. s., 1H), 7.21 (d, J=8.23 Hz, 2H), 7.16 (d, J=9.88 Hz, 1H), 5.42-5.12 (m, 1H), 4.23-4.00 (m, 2H), 3.10 (t, J=12.08 Hz, 1H), 2.07-1.85 (m, 3H), 1.78-1.56 (m, 2H), 1.08 (d, J=6.59 Hz, 3H), 1.05-1.01 (m, 2H), 0.77 (tt, J=4.63, 2.23 Hz, 2H).

### Example D-84: Synthesis of 5-[(2R,3R)-3-[5-(dimethylamino)pyridine-2-amido]-2-methylpiperidin-1-yl]-3-[(1-methyl-1H-pyrazol-4-yl)amino]pyrazine-2-carboxamide (D-84)

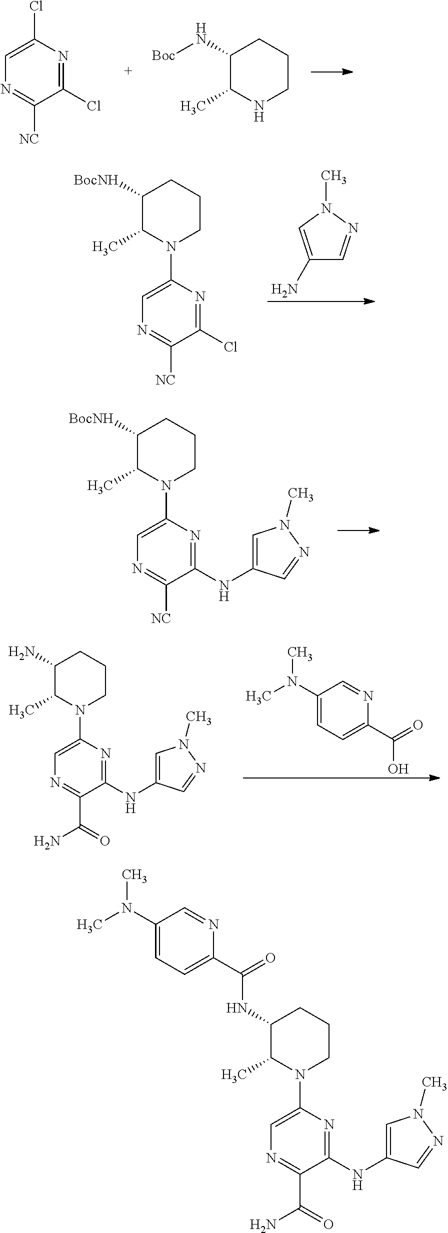

To a solution of 3,5-dichloropyrazine-2-carbonitrile (6.42 g, 36.9 mmol) in DMF (60 mL) was added tert-butyl N-[(2R,3R)-2-methylpiperidin-3-yl]carbamate (7.89 g, 36.9 mmol) and DIPEA (12.8 mL, 73.8 mmol). The mixture was stirred at room temperature overnight. The mixture was poured into an ice/water bath and extracted with ethyl acetate. The organic phasese were collected and dried over Na₂SO₄, filtered and concentrated under vacuo. The crude was purified by flash chromatography with 0% to 70% of ethyl acetate in cyclohexane to give tert-butyl N-[(2R,3R)-1-(6-chloro-5-cyanopyrazin-2-yl)-2-methylpiperidin-3-yl]carbamate (12.25 g, 94% yield). MS found for C16H22ClN5O2 as (M+H)⁺ 352.3.

A mixture of tert-butyl N-[(2R,3R)-1-(6-chloro-5-cyanopyrazin-2-yl)-2-methylpiperidin-3-yl]carbamate (1.5 g, 4.26 mmol), l-methyl-1H-pyrazol-4-amine (1 g, 7.49 mmol), Pd(OAc)₂ (0.19 mg, 0.852 mmol), (+/−) BINAP (0.53 mg, 0.852 mmol), fine powder Cs₂CO₃ (5.55 g, 17.04 mmol) in dioxane (20 mL) was degassed with a nitrogen stream for 10 min. The mixture was stirred in a nitrogen atmosphere at 90° C. for 5 h, then 2 mL of water were added and the mixture was left stirring at 90° C. overnight. Then it was cooled to room temperature, filtered and and concentrated in vacuo. The residue was purified by flash chromatography with 20 to 100% ethyl acetate in cyclohexane to isolate tert-butyl N-[(2R,3R)-1-{5-cyano-6-[(1-methyl-1H-pyrazol-4-yl)amino]pyrazin-2-yl}-2-methylpiperidin-3-yl]carbamate (1.45 g, 83% yield).

MS found for C20H28N8O2 as (M+H)⁺ 413.1.

tert-butyl N-[(2R,3R)-1-(5-cyano-6-{[4-(1-cyclopentyl-4-methylpiperidin-4-yl) phenyl]amino}pyrazin-2-yl)-2-ethylpiperidin-3-yl]carbamate (0.21 g, 0.37 mmol) was dissolved in a mixture of TFA (10 ml) and H₂SO₄ (0.4 mL). The reaction was left stirring at room temperature for 4 h, then concentrated and passed through an SCX cartridge eluting with ammonia in MeOH 2 N. The residue was purified by reverse phase chromatography (H₂O/CH₃CN+0.1% HCOOH from 1/0 to 0/1) to give 5-[(2R,3R)-3-amino-2-methylpiperidin-1-yl]-3-[(1-methyl-1H-pyrazol-4-yl)amino]pyrazine-2-carboxamide (0.95 g, 82% yield). MS found for C5H22N8O as (M+H)⁺ 331.1

5-[(2R,3R)-3-amino-2-methylpiperidin-1-yl]-3-[(1-methyl-1H-pyrazol-4-yl)amino]pyrazine-2-carboxamide (80 mg 0.24 mmol), 5-(dimethylamino)pyridine-2-carboxylic acid (48 mg, 0.29 mmol) and DIPEA (0.12 ml, 0.72 mmol) were dissolved in DMF (3 ml). PyBOP (187 mg, 0.36 mmol) was added and the reaction was left stirring at room temperature overnight. The mixture was concentrated and purified by flash chromatography with 0 to 10% MeOH in DCM to isolate 5-[(2R,3R)-3-[5-(dimethylamino)pyridine-2-amido]-2-methylpiperidin-1-yl]-3-[(1-methyl-1H-pyrazol-4-yl)amino]pyrazine-2-carboxamide (D-84) (17.6 mg 15% yield).MS found for C23H30N10O2 as (M+H)⁺ 475.2.

¹H NMR (500 MHz, DMSO) δ 10.87 (s, 1H), 8.35 (d, J=6.86 Hz, 1H), 8.03 (br. s., 1H), 7.85 (d, J=8.23 Hz, 2H), 7.69 (br. s., 1H), 7.57 (s, 1H), 7.47 (s, 1H), 7.36 (d, J=8.23 Hz, 2H), 7.28 (br. s., 1H), 5.53-5.10 (m, 1H), 4.25-3.97 (m, 2H), 3.77 (s, 3H), 3.04-3.17 (m, 1H), 2.96 (quin, J=6.86 Hz, 1H), 2.08-1.81 (m, 2H), 1.77-1.50 (m, 2H), 1.23 (d, J=6.86 Hz, 6H), 1.09 (d, J=6.86 Hz, 3H).

### Example D-85: Synthesis of 3-[(1-methyl-1H-pyrazol-4-yl)amino]-5-[(2R,3R)-2-methyl-3-[4-(pyrimidin-2-yl)benzamido]piperidin-1-yl]pyrazine-2-carboxamide (D-85)

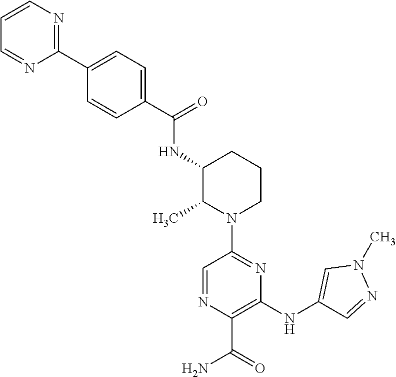 [SMILES: C[C@@H]1[C@H](NC(=O)c2ccc(-c3ncccn3)cc2)CCCN1c1cnc(C(N)=O)c(Nc2cnn(C)c2)n1]

In a similar manner as described in Example D-84, 3-[(1-methyl-1H-pyrazol-4-yl)amino]-5-[(2R,3R)-2-methyl-3-[4-(pyrimidin-2-yl)benzamido]piperidin-1-yl]pyrazine-2-carboxamide (D-85) was prepared using 4-(pyrimidin-2-yl)benzoic acid. MS found for C26H28N10O2 as (M+H)⁺ 513.2.

¹H NMR (400 MHz, DMSO) δ 10.87 (s, 1H), 8.96 (d, J=4.82 Hz, 2H), 8.61 (d, J=6.80 Hz, 1H), 8.50 (d, J=8.33 Hz, 2H), 8.08 (d, J=8.33 Hz, 2H), 8.03 (s, 1H), 7.70 (br. s., 1H), 7.58 (s, 1H), 7.51 (t, J=4.93 Hz, 1H), 7.48 (s, 1H), 7.28 (br. s., 1H), 5.49-5.17 (m, 1H), 4.24-3.98 (m, 2H), 3.76 (s, 3H), 3.10 (t, J=12.94 Hz, 1H), 2.08-1.81 (m, 2H), 1.79-1.52 (m, 2H), 1.13 (d, J=6.80 Hz, 3H).

### Example D-86 5-[(2R,3R)-3-(2-cyclopropylpyrimidine-5-amido)-2-methylpiperidin-1-yl]-3-[(1-methyl-1H-pyrazol-4-yl)amino]pyrazine-2-carboxamide (D-86)

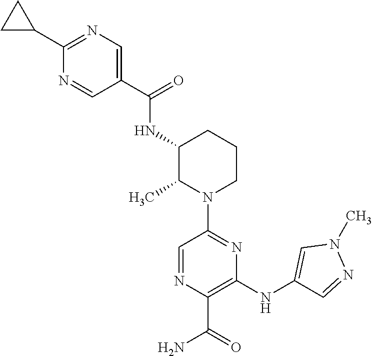 [SMILES: C[C@@H]1[C@H](NC(=O)c2cnc(C3CC3)nc2)CCCN1c1cnc(C(N)=O)c(Nc2cnn(C)c2)n1]

In a similar manner as described in Example D-84, 5-[(2R,3R)-3-(2-cyclopropylpyrimidine-5-amido)-2-methylpiperidin-1-yl]-3-[(1-methyl-1H-pyrazol-4-yl)amino]pyrazine-2-carboxamide (D-86) was prepared using 5-pyrimidinecarboxylic acid, 2-cyclopropyl. MS found for C23H28N10O2 as (M+H)⁺ 477.2.

¹H NMR (500 MHz, DMSO) δ 10.88 (s, 1H) 9.04 (s, 2H), 8.68 (d, J=6.59 Hz, 1H), 8.00 (s, 1H), 7.70 (br. s., 1H), 7.57 (s, 1H), 7.48 (s, 1H), 7.36-7.20 (m, 1H), 5.33 (br. s., 1H), 4.24-3.94 (m, 2H), 3.77 (s, 3H), 3.09 (t, J=12.21 Hz, 1H), 2.33-2.21 (m, 1H), 1.97-1.54 (m, 4H), 1.19-1.03 (m, 7H).

### Example D-87: Synthesis of 3-[(1-methyl-1H-pyrazol-4-yl)amino]-5-[(2R,3R)-2-methyl-3-[5-(trifluoromethyl)pyridine-2-amido]piperidin-1-yl]pyrazine-2-carboxamide (D-87)

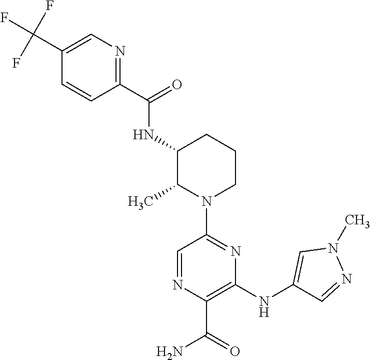 [SMILES: C[C@@H]1[C@H](NC(=O)c2ccc(C(F)(F)F)cn2)CCCN1c1cnc(C(N)=O)c(Nc2cnn(C)c2)n1]

In a similar manner as described in Example D-84, 3-[(1-methyl-1H-pyrazol-4-yl)amino]-5-[(2R,3R)-2-methyl-3-[5-(trifluoromethyl)pyridine-2-amido]piperidin-1-yl]pyrazine-2-carboxamide (D-87) was prepared using 5-(trifluoromethyl)pyridine-2-carboxylic acid. MS found for C22H24F3N9O2 as (M+H)⁺ 504.0.

¹H NMR (400 MHz, DMSO) δ 10.87 (s, 1H), 9.07 (s, 1H), 8.89 (d, J=7.43 Hz, 1H), 8.47 (dd, J=8.22, 1.96 Hz, 1H), 8.27 (d, J=8.22 Hz, 1H), 7.99 (s, 1H), 7.69 (br. s., 1H), 7.58 (s, 1H), 7.48 (s, 1H), 7.28 (br. s., 1H), 5.44-5.15 (m, 1H), 4.20-3.98 (m, 2H), 3.78 (s, 3H), 3.15-2.97 (m, 1H), 2.17-1.52 (m, 4H), 1.10 (d, J=7.04 Hz, 3H).

### Example D-88: Synthesis of 5-[(2R,3R)-3-[4-(1-cyano-1-methylethyl)benzamido]-2-methylpiperidin-1-yl]-3-[(1-methyl-1H-pyrazol-4-yl)amino]pyrazine-2-carboxamide (D-88)

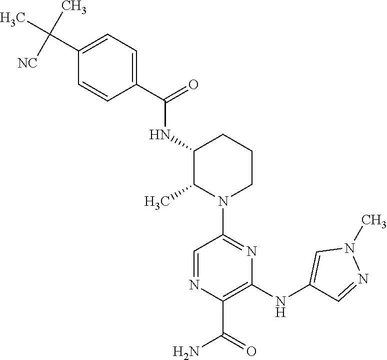 [SMILES: C[C@@H]1[C@H](NC(=O)c2ccc(C(C)(C)C#N)cc2)CCCN1c1cnc(C(N)=O)c(Nc2cnn(C)c2)n1]

In a similar manner as described in Example D-84, 5-[(2R,3R)-3-[4-(1-cyano-1-methylethyl)benzamido]-2-methylpiperidin-1-yl]-3-[(1-methyl-1H-pyrazol-4-yl)amino]pyrazine-2-carboxamide (D-88), was prepared using 4-(2-cyanopropan-2-yl)benzoic acid. MS found for C26H31N9O2 as (M+H)⁺ 477.2.

¹H NMR (500 MHz, DMSO) δ 10.88 (s, 1H), 9.04 (s, 2H), 8.68 (d, J=6.59 Hz, 1H), 8.00 (s, 1H), 7.70 (br. s., 1H), 7.57 (s, 1H), 7.48 (s, 1H), 7.36-7.20 (m, 1H), 5.33 (br. s., 1H), 4.24-3.94 (m, 2H), 3.77 (s, 3H), 3.09 (t, J=12.21 Hz, 1H), 2.33-2.21 (m, 1H), 1.97-1.54 (m, 4H), 1.19-1.03 (m, 7H).

### Example D-89: Synthesis of 5-[(2R,3R)-3-(4-cyclopropylbenzamido)-2-methylpiperidin-1-yl]-3-({4-[(1-methylpiperidin-4-yl)oxy]phenyl}amino)pyrazine-2-carboxamide (D-89)

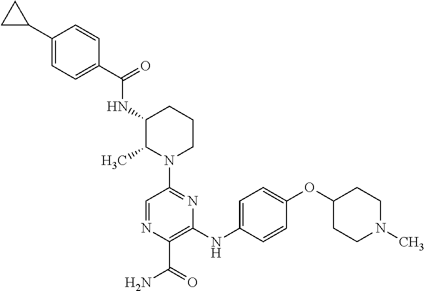 [SMILES: C[C@@H]1[C@H](NC(=O)c2ccc(C3CC3)cc2)CCCN1c1cnc(C(N)=O)c(Nc2ccc(OC3CCN(C)CC3)cc2)n1]

In a similar manner as described in Example D-291, 5-[(2R,3R)-3-(4-cyclopropylbenzamido)-2-methylpiperidin-1-yl]-3-({4-[(1-methylpiperidin-4-yl)oxy]phenyl}amino)pyrazine-2-carboxamide (D-89) was prepared, using cyclopropylbenzoic acid. MS found for C33H41N7O3 as (M+H)⁺ 584.3.

¹H NMR (500 MHz, DMSO) δ 11.05 (s, 1H), 8.31 (d, J=7.13 Hz, 1H), 7.82 (d, J=8.23 Hz, 2H), 7.73 (br. s., 1H), 7.61 (s, 1H), 7.50 (d, J=8.78 Hz, 2H), 7.30 (br. s., 1H), 7.18 (d, J=8.23 Hz, 2H), 6.82 (d, J=8.51 Hz, 2H), 5.37-4.88 (m, 1H), 4.34-3.94 (m, 3H), 3.06 (t, J=12.62 Hz, 1H), 2.58-2.52 (m, 2H), 2.15 (s, 3H), 2.11-1.48 (m, 11H), 1.13-0.97 (m, 5H), 0.78-0.69 (m, 2H).

### Example D-90: Synthesis of 5-[(2R,3R)-3-{bicyclo[1.1.1]pentane-1-amido}-2-methylpiperidin-1-yl]-3-[(1-methyl-1H-pyrazol-4-yl)amino]pyrazine-2-carboxamide (D-90)

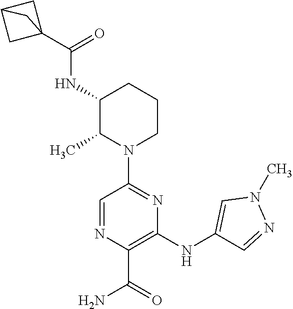 [SMILES: C[C@@H]1[C@H](NC(=O)C23CC(C2)C3)CCCN1c1cnc(C(N)=O)c(Nc2cnn(C)c2)n1]

In a similar manner as described in Example D-84, 5-[(2R,3R)-3-{bicyclo[1.1.1]pentane-1-amido}-2-methylpiperidin-1-yl]-3-[(1-methyl-1H-pyrazol-4-yl)amino]pyrazine-2-carboxamide (D-90) was prepared, using bicyclo[1.1.1]pentane-1-carboxylic acid. MS found for C21H28N8O2 as (M+H)⁺ 425.2.

¹H NMR (400 MHz, DMSO) δ 10.86 (s, 1H), 8.01 (s, 1H), 7.75 (d, J=6.65 Hz, 1H), 7.66 (br. s., 1H), 7.52 (s, 1H), 7.42 (s, 1H), 7.26 (br. s., 1H), 5.22 (br. s., 1H), 4.04 (d, J=10.56 Hz, 1H), 3.86 (s, 3H), 3.81-3.69 (m, 1H), 3.03 (t, J=12.32 Hz, 1H), 2.42 (s, 1H), 2.04-1.94 (m, 6H), 1.89-1.74 (m, 2H), 1.66-1.44 (m, 2H), 0.99 (d, J=6.85 Hz, 3H).

### Example D-91: Synthesis of 3-[(1-methyl-1H-pyrazol-4-yl)amino]-5-[(2R,3R)-2-methyl-3-[6-(trifluoromethyl)pyridine-3-amido]piperidin-1-yl]pyrazine-2-carboxamide (D-91)

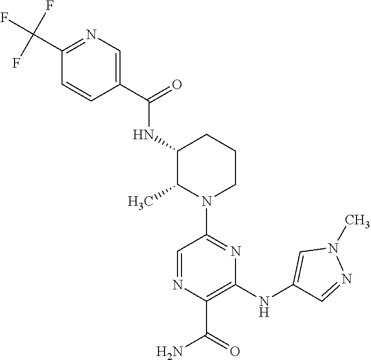 [SMILES: C[C@@H]1[C@H](NC(=O)c2ccc(C(F)(F)F)nc2)CCCN1c1cnc(C(N)=O)c(Nc2cnn(C)c2)n1]

In a similar manner as described in Example D-84, 3-[(1-methyl-1H-pyrazol-4-yl)amino]-5-[(2R,3R)-2-methyl-3-[6-trifluoromethyl)pyridine-3-amido]piperidin-1-yl]pyrizine-2-carboxamide (D-91) was prepared, using 6-(trifluoromethyl)pyridine-3-carboxylic acid. MS found for C22H24F3N9O2 as (M+H)⁺ 504.2.

¹H NMR (500 MHz, DMSO) δ 10.88 (s, 1H), 9.20 (s, 1H), 8.88 (d, J=6.59 Hz, 1H), 8.55-8.50 (m, 1H), 8.08 (d, J=8.23 Hz, 1H), 8.01 (s, 1H), 7.70 (br. s., 1H), 7.58 (s, 1H), 7.49 (s, 1H), 7.29 (br. s., 1H), 5.32 (br. s., 1H), 4.24-3.98 (m, 2H), 3.77 (s, 3H), 3.16-3.05 (m, 1H), 2.06-1.53 (m, 4H), 1.13 (d, J=6.86 Hz, 3H).

### Example D-92: Synthesis of 5-[(2R,3R)-3-(4-cyclopropylbenzamido)-2-methylpiperidin-1-yl]-3-{[4-(4-methylpiperazin-1-yl)-3-(trifluoromethyl)phenyl]amino}pyrazine-2-carboxamide (D-92)

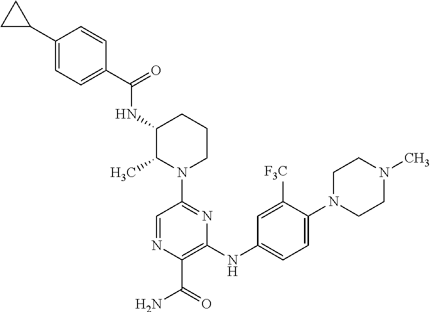 [SMILES: C[C@@H]1[C@H](NC(=O)c2ccc(C3CC3)cc2)CCCN1c1cnc(C(N)=O)c(Nc2ccc(N3CCN(C)CC3)c(C(F)(F)F)c2)n1]

In a similar manner as described in Example 8, 5-[(2R,3R)-3-(4-cyclopropylbenzamido)-2-methylpiperidin-1-yl]-3-{[4-(4-methylpiperazin-1-yl)-3-(trifluoromethyl) phenyl]amino} pyrazine-2-carboxamide (D-92) was prepared, using cyclopropylbenzoic acid and 4-(4-methylpiperazin-1-yl)-3-(trifluoromethyl)aniline. MS found for C33H39F3N8O2 as (M+H)⁺ 637.3.

¹H NMR (400 MHz, DMSO) δ 11.47 (s, 1H), 8.30 (d, J=7.43 Hz, 1H), 8.23-7.95 (m, 1H), 7.88-7.74 (m, 3H), 7.72 (s, 1H), 7.66 (d, J=5.09 Hz, 1H), 7.50-7.36 (m, 2H), 7.16 (d, J=8.22 Hz, 2H), 5.10-4.80 (m, 1H), 4.32-4.12 (m, 1H), 4.10-4.00 (m, 1H), 3.08 (t, J=11.74 Hz, 1H), 2.74 (br. s., 4H), 2.47-2.29 (m, 4H), 2.21 (s, 3H), 2.05-1.76 (m, 3H), 1.72-1.48 (m, 2H), 1.13 (d, J=6.65 Hz, 3H), 1.06-0.98 (m, 2H), 0.80-0.71 (m, 2H).

### Example D-93: Synthesis of 3-[(1-methyl-1H-pyrazol-4-yl)amino]-5-[(2R,3R)-2-methyl-3-(3-methyl-2-oxoimidazolidin-1-yl)piperidin-1-yl]pyrazine-2-carboxamide (D-93)

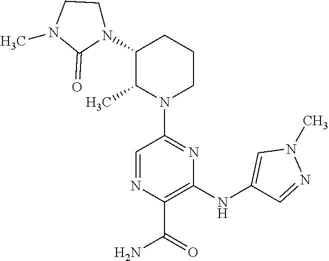

In a similar manner as described in Example 52, 3-[(1-methyl-1H-pyrazol-4-yl)amino]-5-[(2R,3R)-2-methyl-3-(3-methyl-2-oxoimidazolidin-1-yl)piperidin-1-yl]pyrazine-2-carboxamide (D-93) was prepared. MS found for C19H27N9O2 as (M+H)⁺ 414.2.

¹H NMR (500 MHz, DMSO) δ 10.86 (s, 1H), 8.04 (br. s., 1H), 7.68 (br. s., 1H), 7.53 (s, 1H), 7.45 (s, 1H), 7.27 (br. s., 1H), 5.53-4.94 (m, 1H), 4.19-3.96 (m, 1H), 3.85 (s, 3H), 3.72 (dt, J=12.97, 4.36 Hz, 1H), 3.47-3.37 (m, 2H), 3.36-3.22 (m, 2H), 3.03 (t, J=12.35 Hz, 1H). 2.68 (s, 3H), 1.98-1.82 (m, 2H), 1.76 (d, J=10.43 Hz, 1H), 1.57 (q, J=12.99 Hz, 1H), 1.08 (d, J=6.86 Hz, 3H).

### Example D-94: Synthesis of 5-[(2R,3R)-3-[4-(1-hydroxy-2-methylpropan-2-yl)benzamido]-2-methylpiperidin-1-yl]-3-[(1-methyl-1H-pyrazol-4-yl)amino]pyrazine-2-carboxamide (D-94)

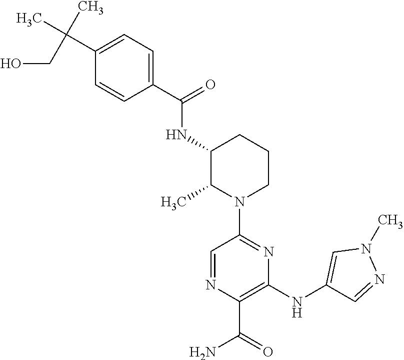 [SMILES: C[C@@H]1[C@H](NC(=O)c2ccc(C(C)(C)CO)cc2)CCCN1c1cnc(C(N)=O)c(Nc2cnn(C)c2)n1]

In a similar manner as described in Example D-216, 5-[(2R,3R)-3-[4-(1-hydroxy-2-methylpropan-2-yl)benzamido]-2-methylpiperidin-1-yl]-3-[(1-methyl-1H-pyrazol-4-yl) amino]pyrazine-2-carboxamide (D-94) was prepared using 4-(1-hydroxy-2-methylpropan-2-yl)benzoic acid. MS found for C26H34N8O3 as (M+H)⁺ 507.1.

¹H NMR (500 MHz, DMSO) δ 10.88 (s, 1H), 8.35 (d, J=6.59 Hz, 1H), 8.04 (br. s., 1H), 7.84 (d, J=8.51 Hz, 2H), 7.69 (br. s., 1H), 7.57 (s, 1H), 7.51-7.43 (m, 3H), 7.28 (br. s., 1H), 5.60-5.09 (m, 1H), 4.71 (t, J=5.35 Hz, 1H), 4.24-3.94 (m 2H),3.78 (s, 3H), 3.45 (d, J=5.49 Hz, 2H), 3.16-3.00 (m, 1H), 1.98-1.90 (m, 1H), 1.86 (d, J=13.17 Hz, 1H), 1.75-1.67 (m, 1H), 1.60 (dt, J=13.10, 4.15 Hz, 1H), 1.25 (s, 6H), 1.09 (d, J=6.86 Hz, 3H).

### Example D-95: Synthesis of 3-[(1-methyl-1H-pyrazol-4-yl)amino]-5-[(2R,3R)-2-methyl-3-[4-(trifluoromethoxy)benzamido]piperidin-1-yl]pyrazine-2-carboxamide (D-95)

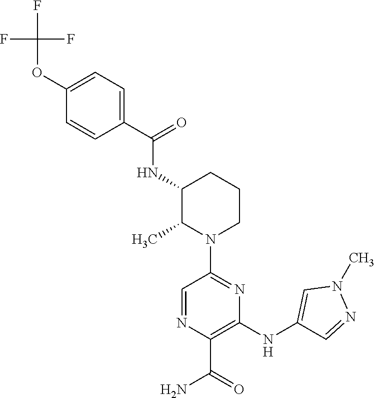 [SMILES: C[C@@H]1[C@H](NC(=O)c2ccc(OC(F)(F)F)cc2)CCCN1c1cnc(C(N)=O)c(Nc2cnn(C)c2)n1]

In a similar manner as described in Example D-216, 3-[(1-methyl-1H-pyrazol-4-yl)amino]-5-[(2R,3R)-2-methyl-3-[4-(trifluoromethoxy)benzamido]piperidin-1-yl]pyrazine-2-carboxamide (D-95) was prepared using 4-(trifluoromethoxy)benzoic acid. MS found for C23H25F3N8O3 as (M+H)⁺ 519.2.

¹H NMR (500 MHz, DMSO) δ 10.87 (s, 1H), 8.58 (d, J=6.72 Hz, 1H), 8.13-7.95 (m, 3H), 7.70 (br. s., 1H), 7.57 (s, 1H), 7.53-7.44 (m, 3H), 7.29 (br. s., 1H), 5.31 (br. s., 1H), 4.31-3.96 (m, 2H), 3.76 (s, 3H), 3.09 (t, J=12.21 Hz, 1H), 1.95 (qd, J=12.92, 3.77 Hz, 1H), 1.87 (d, J=13.17 Hz, 1H), 1.72 (d, J=10.02 Hz, 1H), 1.67-1.51 (m, 1H), 1.10 (d, J=6.86 Hz, 3H).

### Example D-96: Synthesis of 5-[(2R,3R)-3-(4-cyclopropylbenzamido)-2-methylpiperidin-1-yl]-3-{[4-(4,4-difluoropiperidin-1-yl)phenyl]amino}pyrazine-2-carboxamide (D-96)

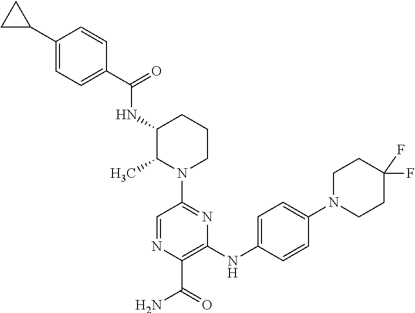 [SMILES: C[C@@H]1[C@H](NC(=O)c2ccc(C3CC3)cc2)CCCN1c1cnc(C(N)=O)c(Nc2ccc(N3CCC(F)(F)CC3)cc2)n1]

In a similar manner as described in Example 8, 5-[(2R,3R)-3-(4-cyclopropylbenzamido)-2-methylpiperidin-1-yl]-3-{[4-(4,4-difluoropiperidin-1-yl)phenyl]amino}pyrazine-2-carboxamide (D-96) was prepared using 4-(4,4-difluoropiperidin-1-yl)aniline (commercial available). MS found for C32H37F2N702 as (M+H)⁺ 590.3.

¹H NMR (500 MHz, DMSO) δ 10.98 (br. s., 1H), 8.35 (d, J=7.41 Hz, 1H), 7.84 (d, J=8.23 Hz, 2H), 7.71 (br. s., 1H), 7.60 (s, 1H), 7.47 (d, J=8.92 Hz, 2H), 7.29 (br. s., 1H), 7.18 (d, J=8.37 Hz, 2H), 6.86 (d, J=7.96 Hz, 2H), 5.14 (br. s., 1H), 4.26-3.99 (m, 2H), 3.19-2.97 (m, 5H), 2.12-1.78 (m, 7H), 1.76-1.49 (m, 2H), 1.12-0.98 (m, 5H), 0.81-0.68 (m, 2H).

### Example D-97: Synthesis of 5-[(2R,3R)-3-(4-cyclopropylbenzamido)-2-methylpiperidin-1-yl]-3-({4-[4-(2,2,2-trifluoroethyl)piperazin-1-yl]phenyl}amino)pyrazine-2-carboxamide (D-97)

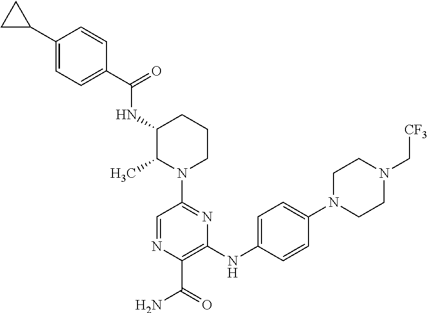 [SMILES: C[C@@H]1[C@H](NC(=O)c2ccc(C3CC3)cc2)CCCN1c1cnc(C(N)=O)c(Nc2ccc(N3CCN(CC(F)(F)F)CC3)cc2)n1]

In a similar manner as described in Example 8, 5-[(2R,3R)-3-(4-cyclopropylbenzamido)-2-methylpiperidin-1-yl]-3-({4-[4-(2,2,2-trifluoroethyl)piperazin-1-yl]phenyl}amino) pyrazine-2-carboxamide (D-97) was prepared using 4-[4-(2,2,2-trifluoroethyl)piperazin-1-yl]aniline. MS found for C33H39F3N8O2 as (M+H)⁺ 637.2.

¹H NMR (500 MHz, DMSO) δ 10.95 (br. s., 1H), 8.35 (d, J=7.41 Hz, 1H), 7.84 (d, J=8.23 Hz, 2H), 7.76-7.65 (m, 1H), 7.59 (s, 1H), 7.45 (d, J=9.06 Hz, 2H), 7.23-7.32 (m, 1H), 7.17 (d, J=8.51 Hz, 2H), 6.72-6.87 (m, 2H), 4.84-5.50 (m, 1H), 4.08 (dd, J=12.08, 7.41 Hz, 2H), 3.21 (q, J=10.15 Hz, 2H), 3.06 (t, J=12.49 Hz, 1H), 2.92 (br. s., 4H), 2.68 (br. s., 4H), 2.08-1.49 (m, 5H), 1.11-0.96 (m, 5H), 0.83-0.68 (m, 2H).

### Preparation of 4-{[1-(2,2,2-trifluoroethyl)piperidin-4-yl]oxy}aniline

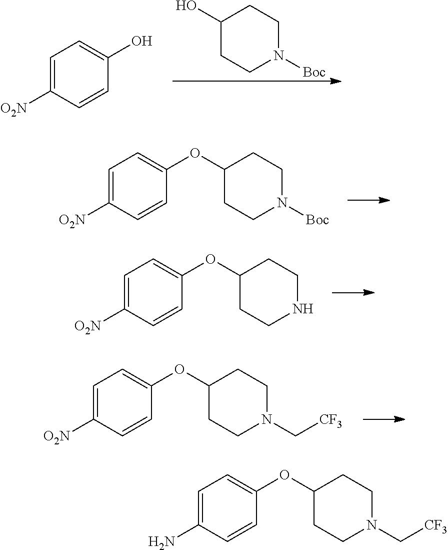

Triphenylphosphine (1.56 g, 5.96 mmol), diisopropyl azadicarboxylate (1.2 ml, 5.96 mmol) and 4-nitrophenol (691 mg, 4.97 mmol) were dissolved in 20 ml of THF at 0° C.; the solution was stirred for 10 min and tert-butyl-4-hydroxypiperidine-1-carboxylate (1 g, 4.97 mmol) was added. The mixture was left to warm up to room temperature and stirred for further 30 min. More triphenylphosphine (2.98 mmol), diisopropyl azadicarboxylate (2.98 mmol) and tert-butyl-4-hydroxypiperidine-1-carboxylate (2.98 mmol), were added to the reaction, and the mixture was left stirring 2 h. Water was added and the reaction was extracted with Et₂O and the organics were washed with brine. The solid was filtered out and the solvent was removed in vacuo. The crude was purified by flash chromatography with 20% of ethyl acetate in cyclohexane to isolate tert-butyl 4-(4-nitrophenoxy)piperidine-1-carboxylate (1.63 g, 99% yield). MS found for C16H22N2O5 as (M+H)⁺ 323.20.

tert-butyl 4-(4-nitrophenoxy)piperidine-1-carboxylate (1.63 g, 4.97 mmol) was dissolved in a mixture of DCM (25 mL) and TFA (5 mL). The reaction was left stirring at room temperature 8 h, then the mixture was concentrated in vacuo and the residue was passed through an SCX cartridge eluting with NH₃ 7N in MeOH. The solvent was removed in vacuo to give 4-(4-nitrophenoxy)piperidine (1.062 g, 96% yield). MS found for C11H14N2O3 as (M+H)⁺ 222.93.

4-(4-nitrophenoxy)piperidine (300 mg, 1.35 mmol), 1,1,1-trifluoro-2-iodoethane (340 mg, 1.62 mmol) and TEA (0.23 mL, 1.62 mmol) were dissolved in DMSO (5.5 ml). The reaction was stirred at 100° C. overnight and at 120° C. further 4 h. The reaction was diluted with water and extracted with Et₂O. The organics were dried over Na₂SO₄ and concentrated under reduced pressure. The crude was purified by flash chromatography with 0% to 20% of ethyl acetate in cyclohexane to isolate 4-(4-nitrophenoxy)-1-(2,2,2-trifluoroethyl)piperidine (126 mg, 31% yield). MS found for C13H15F3N2O3 as (M+H)⁺ 305.18.

4-(4-nitrophenoxy)-1-(2,2,2-trifluoroethyl)piperidine (126 mg, 0.41 mmol) was suspended in EtOH. Pd/C 10 wt % (25 mg) was added and the reaction was left stirring under H₂ atmosphere overnight. The catalyst was filtered out and the filtrated was evaporated to isolate 4-{[1-(2,2,2-trifluoroethyl)piperidin-4-yl]oxy}aniline (108 mg, 95% yield). MS found for C13H17F3N2O as (M+H)⁺ 275.1.

### Example D-98: Synthesis of 5-[(2R,3R)-3-(4-cyclopropylbenzamido)-2-methylpiperidin-1-yl]-3-[(4-{[1-(2,2,2-trifluoroethyl)piperidin-4-yl]oxy}phenyl)amino]pyrazine-2-carboxamide (D-98)

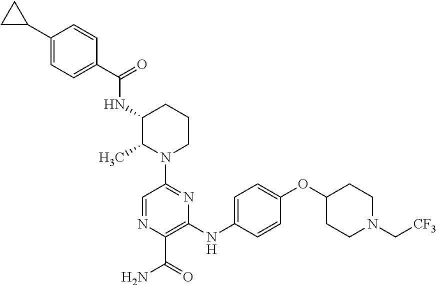 [SMILES: C[C@@H]1[C@H](NC(=O)c2ccc(C3CC3)cc2)CCCN1c1cnc(C(N)=O)c(Nc2ccc(OC3CCN(CC(F)(F)F)CC3)cc2)n1]

In a similar manner as described in Example D-291, 5-[(2R,3R)-3-(4-cyclopropylbenzamido)-2-methylpiperidin-1-yl]-3-[(4-{[1-(2,2,2-trifluoroethyl)piperidin-4-yl]oxy}phenyl)amino]pyrazine-2-carboxamide (D-98) was prepared using 4-{[1-(2,2,2-trifluoroethyl)piperidin-4-yl]oxy}aniline. MS found for C34H40F3N7O3 as (M+H)⁺ 652.4.

¹H NMR (400 MHz, DMSO) δ 11.05 (s, 1H), 8.30 (d, J=7.04 Hz, 1H), 7.82 (d, J=8.22 Hz, 2H), 7.72 (br. s., 1H), 7.61 (s, 1H), 7.50 (d, J=8.61 Hz, 2H), 7.29 (br.s., 1H), 7.18 (d, J=8.22 Hz, 2H), 6.83 (d, J=9.00 Hz, 2H), 5.29-4.93 (m, 1H), 4.24-3.96 (m, 3H), 3.15 (q. J=10.17 Hz, 2H), 3.06 (t, J=12.13 Hz, 1H), 2.78 (br. s., 2H), 2.50 (s, 2H), 2.05-1.73 (m, 5H), 1.72-1.47 (m, 4H), 1.13-0.96 (m, 5H), 0.69-0.80 (m, 2H).

### Preparation of 7-(4-nitrophenyl)-2-oxa-7-azaspiro[3.5]nonane

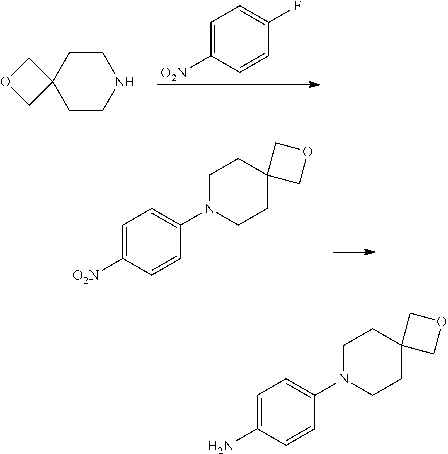 [SMILES: C1CC2(CCN1)COC2 ; O=[N+]([O-])c1ccc(F)cc1 ; O=[N+]([O-])c1ccc(N2CCC3(CC2)COC3)cc1 ; Nc1ccc(N2CCC3(CC2)COC3)cc1]

A mixture of 2-oxa-7-azaspiro[3,5]nonane (150 mg, 1.2 mmol), 1-fluoro-4-nitrobenzene (183 mg, 1.3 mmol), K₂CO₃ (331.7 mg, 2.4 mmol) in DMF (8 mL) was left stirring at room temperature for 8 h and further 8 hours at 50° C. Water was added to the mixture, a precipitate occurred. It was filtered and dried to isolate 7-(4-nitrophenyl)-2-oxa-7-azaspiro[3.5]nonane (194 mg, 65% yield). MS found for C13H16N2O3 as (M+H)⁺ 249.0.

7-(4-nitrophenyl)-2-oxa-7-azaspiro[3.5]nonane (194 mg, 0.79 mmol) was suspended in 10 ml of EtOH. Pd/C 10 wt % (30 mg) was added and the reaction was left stirring under H₂ atmosphere 8 h. The catalyst was filtered out and the filtrated was evaporated to give 4-{2-oxa-7-azaspiro[3.5]nonan-7-yl}aniline (168 mg, 97.4% yield). MS found for C₁₃H₁₈N₂O as (M+H)⁺ 219.2.

### Example D-99: Synthesis of 5-[(2R,3R)-3-(4-cyclopropylbenzamido)-2-methylpiperidin-1-yl]-3-[(4-{2-oxa-7-azaspiro[3.5]nonan-7-yl}phenyl)amino]pyrazine-2-carboxamide (D-99)

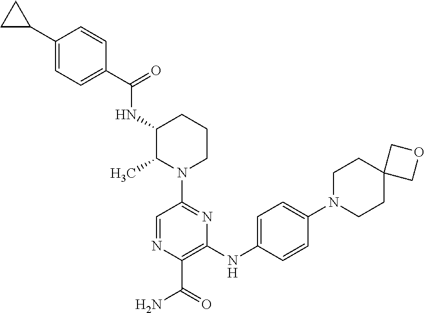 [SMILES: C[C@@H]1[C@H](NC(=O)c2ccc(C3CC3)cc2)CCCN1c1cnc(C(N)=O)c(Nc2ccc(N3CCC4(CC3)COC4)cc2)n1]

In a similar manner as described in Example 8, 5-[(2R,3R)-3-(4-cyclopropylbenzamido)-2-methylpiperidin-1-yl]-3-[(4-{2-oxa-7-azaspiro[3.5]nonan-7-yl}phenyl)amino]pyrazine-2-carboxamide (D-99) was prepared using 4-{2-oxa-7-azaspiro[3.5]nonan-7-yl}aniline. MS found for C34H41N7O3 as (M+H)⁺ 594.3.

¹H NMR (400 MHz, DMSO) δ 10.93 (s, 1H), 8.34 (d, J=7.63 Hz, 1H), 7.84 (d, J=8.22 Hz, 2H), 7.69 (br. s., 1H), 7.58 (s, 1H), 7.43 (d, J=9.00 Hz, 2H), 7.27 (br. s., 1H), 7.20 (d, J=8.22 Hz, 2H), 6.79 (d, J=8.61 Hz, 2H), 5.13 (br. s., 1H), 4.35-4.26 (m, 4H), 4.19-3.97 (m, 2H), 3.06 (t, J=11.74 Hz, 1H), 2.83 (br. s., 4H), 2.14-1.74 (m, 7H), 1.73-1.46 (m, 2H), 1.12-0.98 (m, 5H), 0.82-0.71 (m, 2H).

### Preparation of 2-[1-(4-aminophenyl)piperidin-4-yl]propan-2-ol

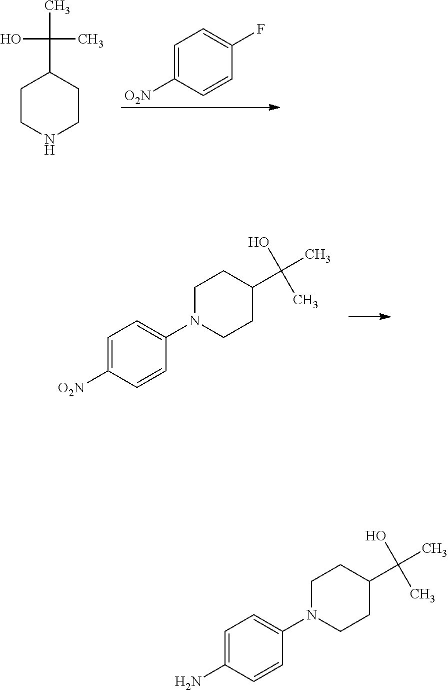

A mixture of 2-(piperidin-1-yl)propan-2-ol (300 mg, 2.08 mmol), 1-fluoro-4-nitrobenzene (323 mg, 2.29 mmol), K₂CO₃ (575 mg, 4.16 mmol) in DMF (14 mL) was left stirring at room temperature for 8 h and further 8 h at 50° C. Water was added to the mixture and a precipitate occurred. It was filtered and dried to isolate 2-[1-(4-nitrophenyl)piperidin-1-yl)propan-2-ol (494 mg, 81.5% yield). MS found for C13H19N3O3 as (M+H)⁺ 265.21.

2-[1-(4-nitrophenyl)piperidin-1-yl)propan-2-ol (494 mg, 1.86 mmol) was suspended in 40 mL of EtOH. Pd/C 10 wt % (100 mg) was added and the reaction was left stirring under H₂ atmosphere 4 h. The catalyst was filtered out and the filtrated was evaporated to isolate 2-[1-(4-aminophenyl)piperidin-4-yl]propan-2-ol (300 mg, 1.3 mmol). MS found for C14H2N2O as (M+H)⁺ 235.3.

### Example D-100: Synthesis of 5-[(2R,3R)-3-(4-cyclopropylbenzamido)-2-methylpiperidin-1-yl]-3-({4-[4-(2-hydroxypropan-2-yl)piperidin-1-yl]phenyl}amino)pyrazine-2-carboxamide (D-100)

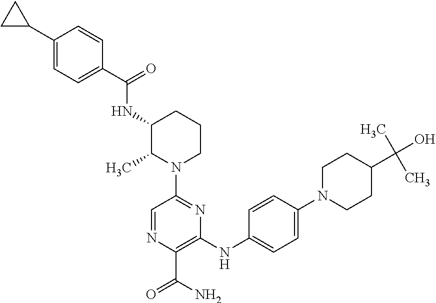 [SMILES: C[C@@H]1[C@H](NC(=O)c2ccc(C3CC3)cc2)CCCN1c1cnc(C(N)=O)c(Nc2ccc(N3CCC(C(C)(C)O)CC3)cc2)n1]

In a similar manner as described in Example 8, 5-[(2R,3R)-3-(4-cyclopropylbenzamido)-2-methylpiperidin-1-yl]-3-({4-[4-(2-hydroxypropan-2-yl)piperidin-1-yl]phenyl}amino)pyrazine-2-carboxamide (D-100) was prepared using 2-[1-(4-aminophenyl)piperidin-4-yl]propan-2-ol. MS found for C35H45N7O3 as (M+H)⁺ 612.4.

¹H NMR (500 MHz, DMSO) δ 10.93 (br. s., 1H), 8.34 (d, J=7.41 Hz, 1H), 7.85 (d, J=7.96 Hz, 2H), 7.70 (br. s., 1H), 7.58 (s, 1H), 7.43 (d, J=8.78 Hz, 2H), 7.26 (br. s., 1H), 7.17 (d, J=8.23 Hz, 2H), 6.79 (d, J=8.23 Hz, 2H), 5.34-4.89 (m, 1H), 4.22-3.98 (m, 3H), 3.64-3.42 (m, 2H), 3.06 (t, J=13.17 Hz, 1H), 2.45-2.30 (m, 2H), 2.05-1.51 (m, 7H), 1.35-1.17 (m, 3H), 1.11-0.97 (m, 11H), 0.79-0.71 (m, 2H).

### Example D-101: Synthesis of 3-({4-[(1-cyclopentylpiperidin-4-yl)oxy]phenyl}amino)-5-[(2R,3R)-3-(4-cyclopropylbenzamido)-2-methylpiperidin-1-yl]pyrazine-2-carboxamide (D-101)

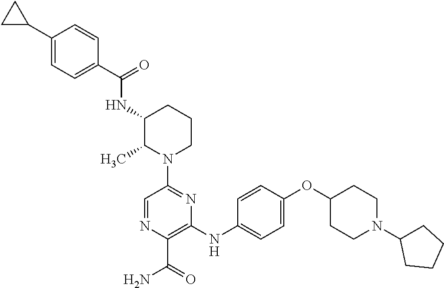 [SMILES: C[C@@H]1[C@H](NC(=O)c2ccc(C3CC3)cc2)CCCN1c1cnc(C(N)=O)c(Nc2ccc(OC3CCN(C4CCCC4)CC3)cc2)n1]

In a similar manner as described in Example D-291, 3-({4-[(1-cyclopentylpiperidin-4-yl)oxy]phenyl}amino)-5-[(2R,3R)-3-(4-cyclopropylbenzamido)-2-methylpiperidin-1-yl]pyrazine-2-carboxamide (D-101) was prepared. MS found for C37H47N7O3 as (M+H)⁺ 638.4.

¹H NMR (400 MHz, DMSO) δ 11.04 (s, 1H), 8.28 (d, J=7.04 Hz, 1H), 7.81 (d, J=8.61 Hz, 2H), 7.70 (br. s., 1H), 7.59 (s, 1H), 7.49 (d, J=9.00 Hz, 2H), 7.28 (br. s., 1H), 7.16 (d, J=8.22 Hz, 2H), 6.81 (d, J=9.00 Hz, 2H), 5.17 (br. s., 1H), 4.22-3.86 (m, 3H), 3.14-2.96 (m, 1H), 2.76-2.59 (m, 2H), 2.56-2.40 (m, 1H), 2.17-1.19 (m, 19H), 1.10-0.94 (m, 5H), 0.78-0.65 (m, 2H).

### Example D-102: Synthesis of 3-({4-[(1-cyclopentylpiperidin-4-yl)oxy]phenyl}amino)-5-[(2R,3R)-3-[(dimethylcarbamoyl)amino]-2-methylpiperidin-1-yl]pyrazine-2-carboxamide (D-102)

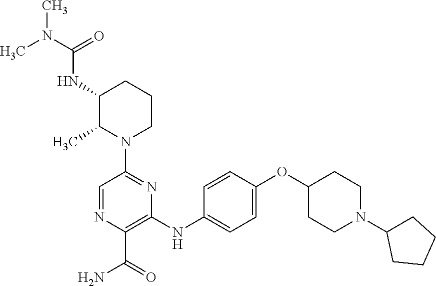 [SMILES: C[C@@H]1[C@H](NC(=O)N(C)C)CCCN1c1cnc(C(N)=O)c(Nc2ccc(OC3CCN(C4CCCC4)CC3)cc2)n1]

In a similar manner as described in Example D-291, 3-({4-[(1-cyclopentylpiperidin-4-yl)oxy]phenyl}amino)-5-[(2R,3R)-3-[(dimethylcarbamoyl)amino]-2-methylpiperidin-1-yl]pyrazine-2-carboxamide (D-102) was prepared. MS found for C30H44N8O3 as (M+H)⁺ 565.5.

¹H NMR (400 MHz, DMSO) δ 11.09 (s, 1H), 7.71 (br. s., 1H), 7.57 (s, 1H), 7.50 (d, J=8.77 Hz, 2H), 7.28 (br. s., 1H), 6.88 (d, J=8.77 Hz, 2H), 6.09 (d, J=7.02 Hz, 1H), 5.11-4.78 (m, 1H), 4.35-4.04 (m, 2H), 3.69 (d, J=4.82 Hz, 1H), 3.07-2.92 (m, 1H), 2.85 (s, 6H), 2.80-2.70 (m, 2H), 2.18 (br. s., 2H), 2.01-1.68 (m, 7H), 1.67-1.41 (m, 8H), 1.40-1.28 (m, 2H), 1.03 (d, J=6.80 Hz, 3H).

### Example D-103: Synthesis of 5-[(2R,3R)-3-(4-cyclopropylbenzamido)-2-methylpiperidin-1-yl]-3-{[2-fluoro-4-(4-methylpiperazin-1-yl)phenyl]amino}pyrazine-2-carboxamide (D-103)

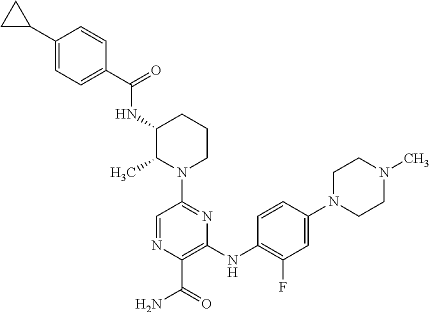 [SMILES: C[C@@H]1[C@H](NC(=O)c2ccc(C3CC3)cc2)CCCN1c1cnc(C(N)=O)c(Nc2ccc(N3CCN(C)CC3)cc2F)n1]

In a similar manner as described in Example 8, 5-[(2R,3R)-3-(4-cyclopropylbenzamido)-2-methylpiperidin-1-yl]-3-{[2-fluoro-4-(4-methylpiperazin-1-yl)phenyl]amino}pyrazine-2-carboxamide (D-103) was prepared using 2-fluoro-4-(4-methylpiperazin-1-yl)aniline. MS found for C32H39FN8O2 as (M+H)⁺ 587.4.

¹H NMR (500 MHz, DMSO) δ 11.00 (s, 1H), 8.35 (d, J=7.41 Hz, 1H), 8.13 (t, J=9.33 Hz, 1H), 7.83 (d, J=8.23 Hz, 2H), 7.72 (br. s., 1H), 7.63 (s, 1H), 7.30 (br. s., 1H), 7.17 (d, J=8.23 Hz, 2H), 6.80 (dd, J=14.27, 1.92 Hz, 1H), 6.55 (br. s., 1H), 5.37-4.91 (m, 1H), 4.25-3.95 (m, 2H), 3.07 (t, J=12.62 Hz, 1H), 2.92 (br. s., 4H), 2.42-2.29 (m, 4H), 2.20 (s, 3H), 2.04-1.97 (m, 1H), 1.97-1.52 (m, 4H), 1.10-0.97 (m, 5H), 0.80-0.71 (m, 2H).

### Example D-104: Synthesis of 5-[(2R,3R)-3-(4-cyclopropylbenzamido)-2-methylpiperidin-1-yl]-3-[(4-{[(1R,5S)-8-methyl-8-azabicyclo[3.2.1]octan-3-yl]oxy}phenyl)amino]pyrazine-2-carboxamide (D-104)

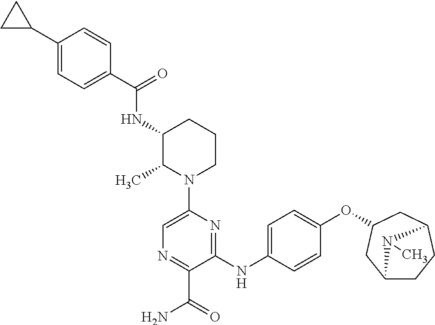

In a similar manner as described in Example D-291, 5-[(2R,3R)-3-(4-cyclopropylbenzamido)-2-methylpiperidin-1-yl]-3-(4-{[(1R,5S)-8-methyl-8-azabicyclo[3.2.1]octan-3-yl]oxy} phenyl) amino]pyrazine-2-carboxamide (D-104) was prepared using 4-{[(1R,3S,5S)-8-methyl-8-azabicyclo[3.2.1]octan-3-yl]oxy}aniline. MS found for C35H43N7O3 as (M+H)⁺ 608.4.

¹H NMR (500 MHz, DMSO) δ 11.08 (s, 1H), 8.34-8.25 (m, 1H), 7.80 (d, J=7.83 Hz, 2H), 7.73 (br. s., 1H), 7.61 (s, 1H), 7.48 (d, J=8.31 Hz, 2H), 7.30 (br. s., 1H), 7.17 (d, J=8.31 Hz, 2H), 6.81 (d, J=8.31 Hz, 2H), 5.12 (br. s., 1H), 4.34 (br. s., 1H), 4.24-3.93 (m, 2H), 3.13-2.99 (m, 3H), 2.20 (s, 3H), 2.03-1.97 (m, 1H), 1.96-1.37 (m, 12H), 1.09 (d, J=6.85 Hz, 3H), 1.04-0.99 (m, 2H), 0.78-0.69 (m, 2H).

### Example D-105: Synthesis of 5-[(2R,3R)-3-(4-cyclopropylbenzamido)-2-methylpiperidin-1-yl]-3-({4-[(1-methylpiperidin-3-yl)oxy]phenyl}amino)pyrazine-2-carboxamide (D-105)

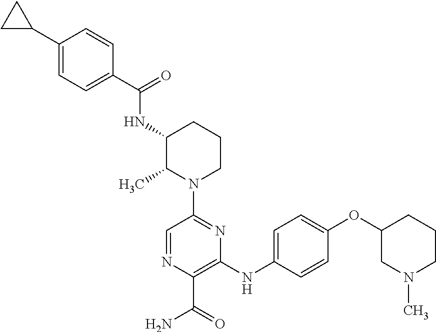 [SMILES: C[C@@H]1[C@H](NC(=O)c2ccc(C3CC3)cc2)CCCN1c1cnc(C(N)=O)c(Nc2ccc(OC3CCCN(C)C3)cc2)n1]

In a similar manner as described in Example D-291, 5-[(2R,3R)-3-(4-cyclopropyl benzamido)-2-methylpiperidin-1-yl]-3-(4-[(1-methylpiperidin-3-yl)oxy] phenyl) amino) pyrazine-2-carboxamide (D-105) was prepared using 4-[(1-methylpiperidin-3-yl)oxy]aniline. MS found for C₃₃H₄₁N₇O₃ as (M+H)⁺ 584.2.

¹H NMR (500 MHz, DMSO) 11.07 (s, 1H), 8.31 (d, J=7.41 Hz, 1H), 7.83 (dd, J=8.10, 5.35 Hz, 2H), 7.73 (br. s., 1H), 7.61 (s, 1H), 7.51 (dd, J=8.78, 2.20 Hz, 2H), 7.30 (br. s., 1H), 7.18 (d, J=8.23 Hz, 2H), 6.83 (d, J=8.51 Hz, 2H), 5.42-4.85 (m, 1H), 4.16 (br. s., 2H), 4.08-4.00 (m, 1H), 3.05 (t, J=12.90 Hz, 1H), 2.85-2.45 (m, 2H), 2.19-2.10 (m, 3H), 2.06-1.22 (m, 11H), 1.11-0.94 (m, 5H), 0.78-0.65 (m, 2H).

### Example D-106: Synthesis of 5-[(2R,3R)-3-(4-cyclopropylbenzamido)-2-methylpiperidin-1-yl]-3-[(4-{[(1R,5S)-8-methyl-8-azabicyclo[3.2.1]octan-3-yl]oxy}phenyl)amino]pyrazine-2-carboxamide (D-106)

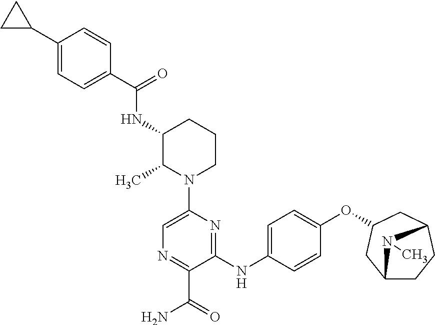

In a similar manner as described in Example D-291, 5-[(2R,3R)-3-(4-cyclopropyl benzamido)-2-methylpiperidin-1-yl]-3-[(4-{[(1R,5S)-8-methyl-8-azabicyclo[3.2.1]octan-3-yl]oxy}phenyl)amino]pyrazine-2-carboxamide (D-106) was prepared using 4-{[(1R,3S,5S)-8-methyl-8-azabicyclo[3.2.]octan-3-yl]oxy}aniline. MS found for C35H43N7O3 as (M+H)⁺ 608.4.

¹H NMR (500 MHz, DMSO) δ 11.08 (s, 1H), 8.34-8.25 (m, 1H), 7.80 (d, J=7.83 Hz, 2H). 7.73 (br. s., 1H), 7.61 (s, 1H), 7.48 (d, J=8.31 Hz, 2H), 7.30 (br. s., 1H), 7.17 (d, J=8.31 Hz, 2H), 6.81 (d, J=8.31 Hz, 2H), 5.12 (br. s., 1H), 4.34 (br. s., 1H), 4.24-3.93 (m, 2H), 3.13-2.99 (m, 3H), 2.20 (s, 3H), 2.03-1.97 (m, 1H), 1.96-1.37 (m, 12H), 1.09 (d, J=6.85 Hz, 3H), 1.04-0.99 (m, 2H), 0.78-0.69 (m, 2H).

### Preparation of 7-(4-nitrophenyl)-2-oxa-7-azaspiro[3.5]nonane.

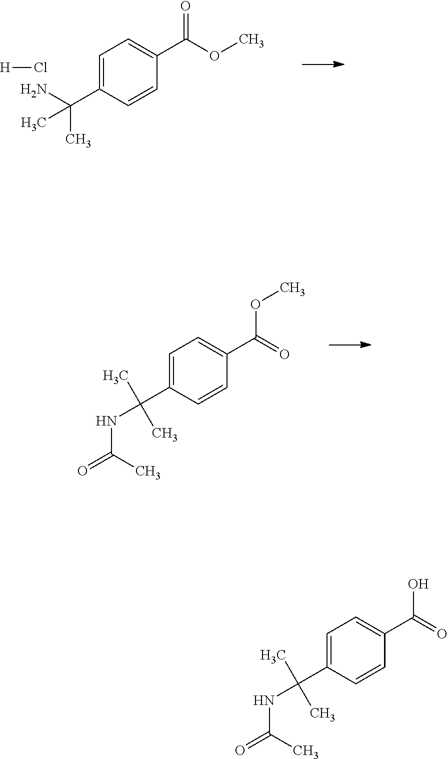

To a solution of methyl 4-(1-amino-1-methylethyl)benzoate hydrochloride (305 mg, 1.33 mmol) in DCM (6 mL) was added tryethylamine (0.463 mL, 3.32 mmol) followed by the addition dropwise of acetyl chloride (0.108 mL, 1.53 mmol) at room temperature. The reaction was left stirring 2 h then quenched with water and extracted with more DCM. The organic layers were dried over Na₂SO₄, filtered and concentrated to give methyl 4-(2-acetamidopropan-2-yl)benzoate (249 mg, 80% yield). MS found for C13H17NO3 as (M+H)⁺ 236.07.

To a mixture of methyl 4-(2-acetamidopropan-2-yl)benzoate (249 mg, 1.06 mmol) in THF (5 mL) and water (1.5 mL), LiOH.H₂O (89 mg, 2.12 mmol) was added and the reaction was left stirring at room temperature overnight. HCl 1N aqueous solution (15 mL) was added and the mixture was extracted with ethyl acetate. The collected organic layers were dried over Na₂SO₄, filtered and concentrated to give 4-(2-acetamidopropan-2-yl)benzoic acid (408.7 mg. 1.06 mmol). MS found for C12H15NO3 as (M+H)⁺ 222.0.

### Example D-107: Synthesis of 5-[(2R,3R)-3-[4-(2-acetamidopropan-2-yl)benzamido]-2-methylpiperidin-1-yl]-3-[(1-methyl-1H-pyrazol-4-yl)amino]pyrazine-2-carboxamide (D-107)

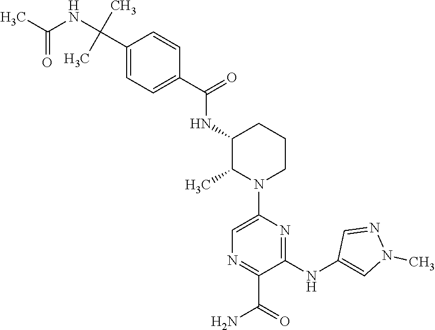 [SMILES: CC(=O)NC(C)(C)c1ccc(C(=O)N[C@@H]2CCCN(c3cnc(C(N)=O)c(Nc4cnn(C)c4)n3)[C@@H]2C)cc1]

In a similar manner as described in Example D-84, 5-[(2R,3R)-3-[4-(2-acetamidopropan-2-yl)benzamido]-2-methylpiperidin-1-yl]-3-[(1-methyl-1H-pyrazol-4-yl)amino]pyrazine-2-carboxamide (D-107) was prepared using 4-(2-acetamidopropan-2-yl)benzoic acid. MS found for C27H35N9O3 as (M+H)⁺ 534.2.

¹H NMR (500 MHz, DMSO) δ 10.88 (s, 1H), 8.35 (d, J=6.85 Hz, 1H), 8.13 (s, 1H), 8.08-7.97 (m, 1H), 7.81 (d, J=8.31 Hz, 2H), 7.70 (br. s., 1H), 7.56 (s, 1H), 7.47 (s, 1H), 7.40 (d, J=8.31 Hz, 2H), 7.28 (br. s., 1H), 5.66-5.00 (m, 1H), 4.25-3.95 (m, 2H), 3.78 (s, 3H), 3.08 (t, J=12.23 Hz, 1H), 1.83 (s, 5H), 1.77-1.46 (m, 8H), 1.09 (d, J=6.85 Hz, 3H).

### Preparation of 7-(4-nitrophenyl)-2-oxa-7-azaspiro[3.5]nonane

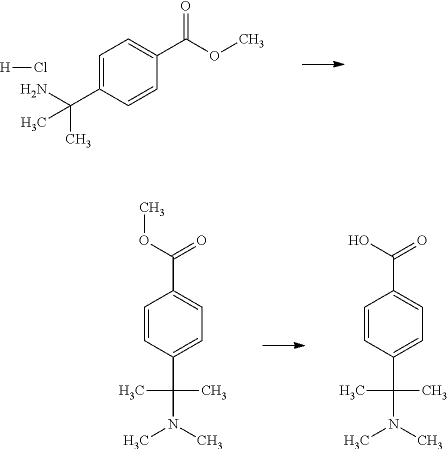

To a solution of methyl 4-(1-amino-1-methylethyl)benzoate hydrochloride (333 mg, 1.45 mmol) in DCM (16 mL) were added formaldehyde solution 37 wt. % in H₂O (0.353 mL, 435 mmol), Na₂SO₄ (105 mg) and sodium triacetoxyborohydride (1.84 g, 8.7 mmol) at room temperature. The reaction was left stirring 2 h then quenched with NaHCO₃ sat sol. DCM was added and the mixture was filtered through a phase separator. The organic layer was removed to give methyl 4-[2-(dimethylamino)propan-2-yl]benzoate (280 mg, 87% yield). MS found for C13H19NO2 as (M+H)⁺ 222.1.

To a mixture of methyl methyl 4-[2-(dimethylamino)propan-2-yl]benzoate (280 mg, 1.27 mmol) in THF (6 mL) and water (1.5 mL), LiOH.H2O (107 mg, 2.54 mmol) was added and the reaction was left stirring at room temperature for 40 h. HCl 6N aqueous solution (0.5 mL) was added and the mixture was extracted with ethyl acetate. The collected organic layers were dried over Na₂SO₄, filtered and concentrated to give 4-[2-(dimethylamino)propan-2-yl]benzoic acid (614 mg, 1.27 mmol). MS found for C12H17NO2 as (M+H)⁺ 208.1.

### Example D-108: Synthesis of 5-[(2R,3R)-3-{4-[2-(dimethylamino)propan-2-yl]benzamido}-2-methylpiperidin-1-yl]-3-[(1-methyl-1H-pyrazol-4-yl)amino]pyrazine-2-carboxamide (D-108)

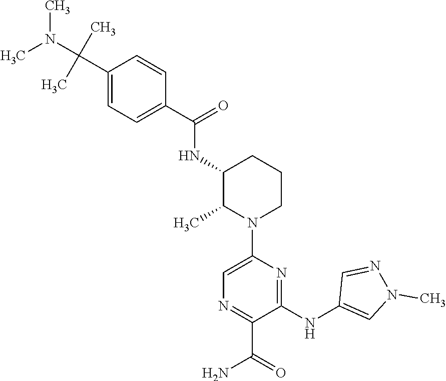

In a similar manner as described in Example D-84, 5-[(2R,3R)-3-{4-[2-(dimethylamino)propan-2-yl]benzamido}-2-methylpiperidin-1-yl]-3-[(1-methyl-1H-pyrazol-4-yl)amino]pyrazine-2-carboxamide (D-108) was prepared using 4-[2-(dimethylamino)propan-2-yl]benzoic acid. MS found for C27H37N9O2 as (M+H)⁺ 520.3

¹H NMR (400 MHz, DMSO) δ 10.87 (s, 1H), 8.38 (d, J=6.80 Hz, 1H), 8.03 (s, 1H), 7.85 (d, J=8.33 Hz, 2H), 7.69 (br. s., 1H), 7.62-7.55 (m, 3H), 7.47 (s, 1H), 7.27 (br. s., 1H), 5.32 (br. s., 1H), 4.29-3.95 (m, 2H), 3.77 (s, 3H), 3.08 (t, J=11.95 Hz, 1H), 2.09 (s, 6H), 2.02-1.52 (m, 4H), 1.30 (s, 6H), 1.10 (d, J=6.80 Hz, 3H).

### Example D-109: Synthesis of 3-[(1-methyl-1H-pyrazol-4-yl)amino]-5-[(2R,3R)-2-methyl-3-methylcarbamoyl)amino]piperidin-1-yl pyrazine-2-carboxamide (D-109)

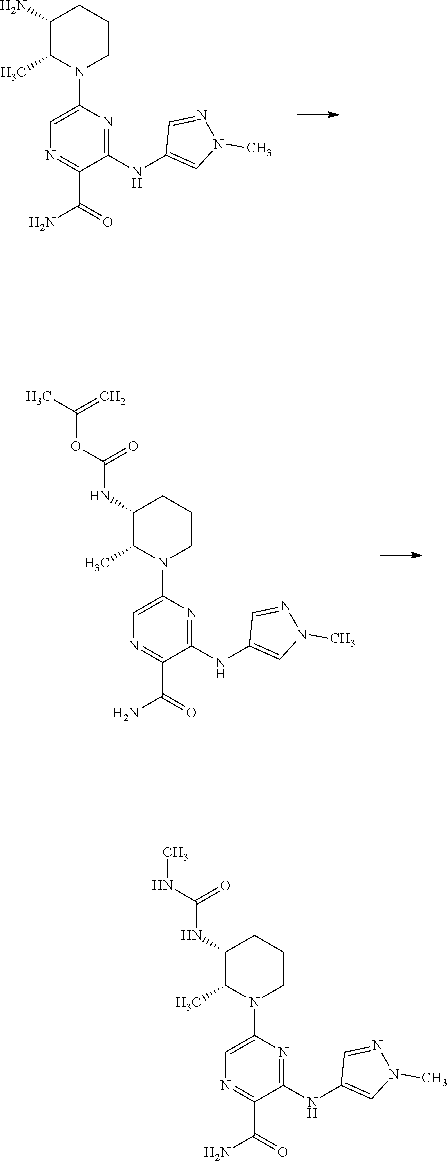 [SMILES: C[C@@H]1[C@H](N)CCCN1c1cnc(C(N)=O)c(Nc2cnn(C)c2)n1 ; C=C(C)OC(=O)N[C@@H]1CCCN(c2cnc(C(N)=O)c(Nc3cnn(C)c3)n2)[C@@H]1C ; CNC(=O)N[C@@H]1CCCN(c2cnc(C(N)=O)c(Nc3cnn(C)c3)n2)[C@@H]1C]

To a solution of 5-[(2R,3R)-3-amino-2-methylpiperidin-1-yl]-3-[(1-methyl-1H-pyrazol-4-yl)amino]pyrazine-2-carboxamide (100 mg, 0.303 mmol) in DCM (4 mL) were added DIPEA (0.106 mL, 0.606 mmol) and isopropenyl chloroformate (0.036 mL, 0.333 mmol) at 0° C. The reaction was left stirring at room temperature overnight NaHCO₃ sat. sol and DCM were added, and the mixture was extracted with DCM. The organic phase was dried and concentrated in vacuo to give prop-1-en-2-yl N-[(2R,3R)-1-{5-carbamoyl-6-(1-methyl-1H-pyrazol-4-yl)amino]pyrazin-2-yl}-2-methylpiperidin-3-yl]carbamate (123 mg, 98% yield). MS found for C19H26N8O3 as (M+H)⁺ 415.14

To a solution of prop-1-en-2-yl N-[(2R,3R)-1-{5-carbamoyl-6-[(1-methyl-1H-pyrazol-4-yl)amino]pyrazin-2-yl}-2-methylpiperidin-3-yl]carbamate (123 mg, 0.297 mmol) in 3 mL of DMF, methanamine 2 M solution in THF (0.594 mL, 1.188 mmol) was added and the reaction was left stirring and heated at 90° C. for 1 h. After this time 1.188 mmol of methanamine 2M solution in THF were added and the reaction was stirred and heated at 100° C. for 1 hour. The solvent was removed in vacuo and the residue purified by silica flash chromatography eluting with MeOH in DCM from 0 to 7% obtaining 3-[(1-methyl-1H-pyrazol-4-yl)amino]-5-[(2R,3R)-2-methyl-3-[(methylcarbamoyl)amino]piperidin-1-yl]pyrazine-2-carboxamide (D-109) (54.8 mg, 47% yield). MS found for C17H25N9O2 as (M+H)⁺ 388.1.

¹H NMR (500 MHz, DMSO) δ 10.85 (s, 1H), 8.03 (s, 1H), 7.67 (br. s., 1H), 7.53 (s, 1H), 7.46 (s, 1H), 7.26 (br. s., 1H), 6.05 (d, J=7.14 Hz, 1H), 5.69 (q, J=4.21 Hz, 1H), 5.14 (br. s., 1H), 4.17-4.01 (m, 1H), 3.86 (s, 3H), 3.68-3.58 (m, 1H), 3.04-2.96 (m, 1H), 2.62-2.56 (m, 3H). 1.87-1.46 (m, 4H), 1.03 (d, J=6.86 Hz, 3H).

### Example D-110: Synthesis of 5-[(2R,3R)-3-[(dimethylcarbamoyl)amino]-2-methylpiperidin-1-yl]-3-{[4-(4-methylpiperazine-1-carbonyl)phenyl]amino}pyrazine-2-carboxamide (D-110)

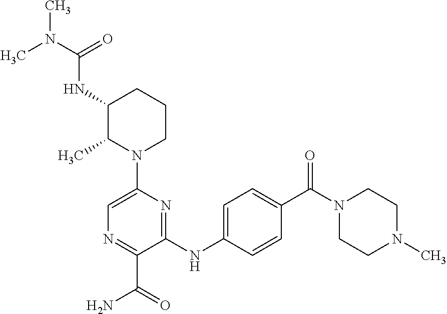

In a similar manner as described in Example 59, 5-[(2R,3R)-3-[(dimethylcarbamoyl)amino]-2-methylpiperidin-1-yl]-3-([4-(4-methylpiperazine-1-carbonyl)phenyl]amino pyrazine-2-carboxamide (D-110) was prepared using 1-[(2R,3R)-1-(6-chloro-5-cyanopyrazin-2-yl)-2-methylpiperidin-3-yl]-3,3-dimethylurea. MS found for C26H37N9O3 as (M+H)⁺ 524.49.

¹H NMR (400 MHz, DMSO) δ 11.51 (s, 1H), 7.80 (d, J=2.20 Hz, 1H), 7.71-7.65 (m, 3H), 7.40 (d, J=2.20 Hz, 1H), 7.35 (d, J=8.78 Hz, 2H), 6.12 (d, J=6.86 Hz, 1H), 5.00 (br. s., 1H), 4.15 (br. s., 1H), 3.75-3.66 (m, 1H), 3.49 (d, J=12.35 Hz, 4H), 3.03 (t, J=12.21 Hz, 1H), 2.85 (s, 6H), 2.30 (br. s., 4H), 2.19 (s, 3H), 1.88-1.74 (m, 2H), 1.67-1.46 (m, 2H), 1.05 (d, J=6.86 Hz, 3H).

### Example D-111: Synthesis of 5-[(2R,3R)-3-[(dimethylcarbamoyl)amino]-2-methylpiperidin-1-yl]-3-{[4-(4-methylpiperazin-1-yl)phenyl]amino}pyrazine-2-carboxamide (D-111)

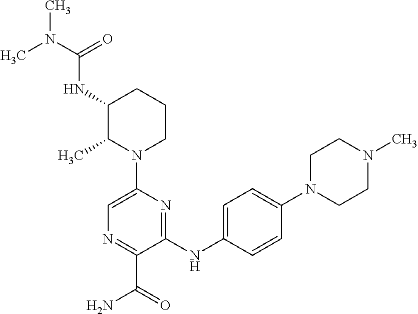

In a similar manner as described in Example 59, 5-[(2R,3R)-3-[(dimethylcarbamoyl)amino]-2-methylpiperidin-1-yl]-3-{[4-(4-methylpiperazin-1-yl)phenyl]amino}pyrazine-2-carboxamide (D-111) was prepared using 1-[(2R,3R)-1-(6-chloro-5-cyanopyrazin-2-yl)-2-methylpiperidin-3-yl]-3,3-dimethylurea. MS found for C25H37N9O2 as (M+H)⁺ 496.52.

¹H NMR (400 MHz, DMSO) δ 11.04 (s, 1H), 7.69 (br. s., 1H), 7.54 (s, 1H), 7.47 (d, J=8.99 Hz, 2H), 7.26 (br. s., 1H), 6.89 (d, J=9.21 Hz, 2H), 6.08 (d, J=7.02 Hz, 1H), 4.93 (br. s., 1H), 4.15 (br. s., 1H), 3.78-3.62 (m, 1H), 3.12-3.02 (m, 4H), 3.03-2.91 (m, 1H), 2.84 (s, 6H), 2.47-2.41 (m, 4H), 2.21 (s, 3H), 1.88-1.44 (m, 4H), 1.04 (d, J=7.02 Hz, 3H).

### Example D-112: Synthesis of 3-{[4-(1-cyclopentyl-4-methylpiperidin-4-yl)phenyl]amino}-5-[(2R,3R)-3-(4-cyclopropylbenzamido)-2-methylpiperidin-1-yl]pyrazine-2-carboxamide (D-112)

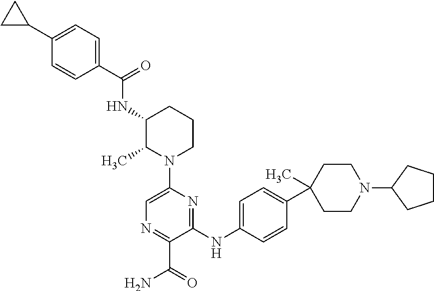 [SMILES: C[C@@H]1[C@H](NC(=O)c2ccc(C3CC3)cc2)CCCN1c1cnc(C(N)=O)c(Nc2ccc(C3(C)CCN(C4CCCC4)CC3)cc2)n1]

In a similar manner as described in Example 65, 3-{[4-(1-cyclopentyl-4-methylpiperidin-4-yl)phenyl]amino}-5-[(2R,3R)-3-(4-cyclopropylbenzamido)-2-methylpiperidin-1-yl]pyrazine-2-carboxamide (D-112) was prepared using N-[(2R,3R)-1-(6-chloro-5-cyanopyrazin-2-yl)-2-methylpiperidin-3-yl]-4-cyclopropylbenzamide. MS found for C38H49N7O2 as (M+H)+ 636.59.

¹H NMR (500 MHz, DMSO) δ 11.18 (br. s., 1H), 8.34 (d, J=6.36 Hz, 1H), 7.85 (d, J=6.60 Hz, 2H), 7.75 (br. s., 1H), 7.64 (d, J=1.96 Hz, 1H), 7.55 (d, J=7.09 Hz, 2H), 7.32 (br. s., 1H), 7.24-7.11 (m, 4H), 5.21 (br. s., 1H), 4.09 (d, J=4.65 Hz, 2H), 3.08 (t, J=12.72 Hz, 1H), 2.45-2.11 (m, 5H), 2.03-1.22 (m, 17H), 1.16-0.91 (m, 8H).

### Example D-113: Synthesis of 5-[(2R,3R)-3-(4-cyclopropyl-2-fluorobenzamido)-2-methylpiperidin-1-yl]-3-({4-[(4-methylpiperazin-1-yl)methyl]phenyl}amino)pyrazine-2-carboxamide (D-113)

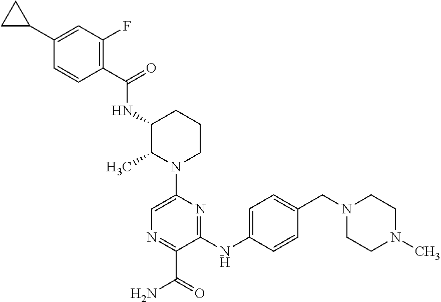 [SMILES: C[C@@H]1[C@H](NC(=O)c2ccc(C3CC3)cc2F)CCCN1c1cnc(C(N)=O)c(Nc2ccc(CN3CCN(C)CC3)cc2)n1]

In a similar manner as described in Example 65, 5-[(2R,3R)-3-(4-cyclopropyl-2-fluorobenzamido)-2-methylpiperidin-1-yl]-3-({4-[(4-methylpiperazin-1-yl)methyl]phenyl}amino)pyrazine-2-carboxamide (D-113) was prepared using N-[(2R,3R)-1-(6-chloro-5-cyanopyrazin-2-yl)-2-methylpiperidin-3-yl]-4-cyclopropyl-2-fluorobenzamide. MS found for C33H41FN8O2 as (M+H)⁺ 601.30.

¹H NMR (400 MHz, DMSO) δ 11.29 (s, 1H), 8.27 (d, J=7.13 Hz, 1H), 7.77 (d, J=2.08 Hz, 1H),7.66 (s, 1H), 7.56 (d, J=8.44 Hz, 2H), 7.51-7.43 (m, 1H), 7.35 (d, J=2.08 Hz, 1H), 7.18 (d, J=8.44 Hz, 2H), 7.06-6.96 (m, 2H), 5.11 (br. s., 1H), 4.16 (d, J=11.84 Hz, 1H), 4.09-3.96 (m, 1H), 3.34 (s, 2H), 3.13-3.00 (m, 1H), 2.44-2.06 (m, 11H), 2.05-1.96 (m, 1H), 1.92-1.76 (m, 2H), 1.72-1.52 (m, 2H), 1.12 (d, J=6.91 Hz, 3H), 1.08-0.99 (m, 2H), 0.80-0.72 (m, 2H).

### Example D-114: Synthesis of 5-[(2R,3R)-3-(4-cyclopropyl-2-fluorobenzamido)-2-methylpiperidin-1-yl]-3-[(quinolin-6-yl)amino]pyrazine-2-carboxamide (D-114)

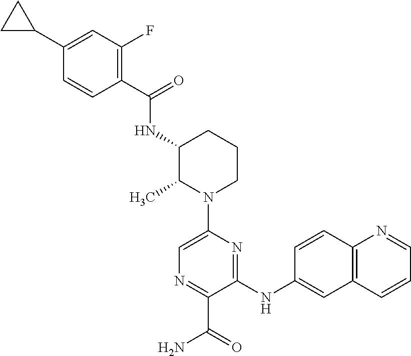 [SMILES: C[C@@H]1[C@H](NC(=O)c2ccc(C3CC3)cc2F)CCCN1c1cnc(C(N)=O)c(Nc2ccc3ncccc3c2)n1]

In a similar manner as described in Example 65, 5-[(2R,3R)-3-(4-cyclopropyl-2-fluorobenzamido)-2-methylpiperidin-1-yl]-3-[(quinolin-6-yl)amino]pyrazine-2-carboxamide (D-114) was prepared using N-[(2R,3R)-1-(6-chloro-5-cyanopyrazin-2-yl)-2-methylpiperidin-3-yl]-4-cyclopropyl-2-fluorobenzamide. MS found for C30H30FN7O2 as (M+H)⁺ 540.56.

¹H NMR (400 MHz, DMSO) δ 11.79 (s, 1H), 8.67 (dd, J=4.11, 1.76 Hz, 1H), 8.48 (d, J=2.35 Hz, 1H), 8.41 (d, J=7.04 Hz, 1H), 8.12 (d, J=8.22 Hz, 1H), 7.92 (d, J=9.00 Hz, 1H), 7.87 (s, 1H), 7.78 (s, 1H), 7.71 (dd, J=9.00, 2.35 Hz, 1H), 7.55-7.44 (m, 2H), 7.17-6.99 (m, 3H), 5.27 (br. s., 1H), 4.33-3.98 (m, 2H), 3.12 (t, J=11.93 Hz, 1H), 2.14-1.97 (m, 1H), 1.89 (d, J=11.35 Hz, 2H), 1.78-1.54 (m, 2H), 1.33-1.13 (m, 3H), 1.11-0.98 (m, 2H), 0.890-0.66 (m, 2H).

### Example D-115: Synthesis of 5-[(2R,3R)-3-(4-cyclopropyl-2-fluorobenzamido)-2-methylpiperidin-1-yl]-3-{[4-(4-methylpiperazine-1-carbonyl)phenyl]amino}pyrazine-2-carboxamide (D-115)

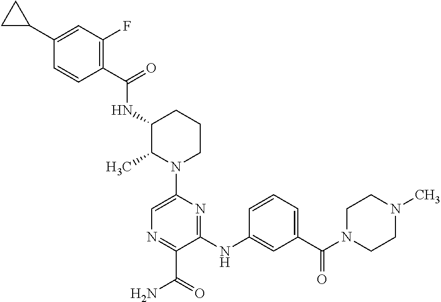 [SMILES: C[C@@H]1[C@H](NC(=O)c2ccc(C3CC3)cc2F)CCCN1c1cnc(C(N)=O)c(Nc2cccc(C(=O)N3CCN(C)CC3)c2)n1]

In a similar manner as described in Example D-216, 5-[(2R,3R)-3-(4-cyclopropyl-2-fluorobenzamido)-2-methylpiperidin-1-yl]-3-{[4-(4-methylpiperazine-1-carbonyl)phenyl]amino}pyrazine-2-carboxamide (D-115) was prepared using 4-cyclopropyl-2-fluorobenzoic acid. MS found for C33H39FN8O3 as (M+H)⁺ 615.63.

¹H NMR (400 MHz, DMSO) δ 11.53 (s, 1H), 8.31 (d, J=7.03 Hz, 1H), 7.81 ((br. s., 1H), 7.74-7.65 (m, 3H), 7.51-7.38 (m, 2H), 7.33 (d, J=8.53 Hz, 2H), 7.06-6.92 (m, 2H), 5.37-5.11 (m, 1H), 4.22-4.09 (m, 1H), 4.07-3.91 (m, 1H), 3.57-3.35 (m, 4H), 3.15-3.02 (m, 1H), 2.20 (m, J=6.50 Hz, 4H), 2.11 (s, 3H), 2.06-1.95 (m, 1H), 1.93-1.78 (m, 2H), 1.74-1.51 (m, 2H), 1.13 (d, J=6.78 Hz, 3H), 1.06-0.99 (m, 2H), 0.80-0.68 (m, 2H).

### Example D-116: Synthesis of 5-[(2R,3R)-3-(4-cyclopropyl-2-fluorobenzamido)-2-methylpiperidin-1-yl]-3-[(3-methyl-1,2-thiazol-5-yl)amino]pyridine-2-carboxamide (D-116)

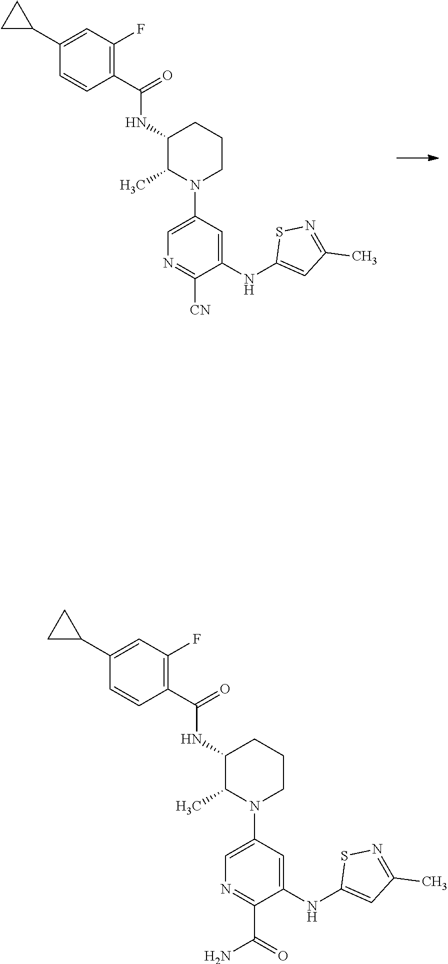 [SMILES: Cc1cc(Nc2cc(N3CCC[C@@H](NC(=O)c4ccc(C5CC5)cc4F)[C@H]3C)cnc2C#N)sn1 ; Cc1cc(Nc2cc(N3CCC[C@@H](NC(=O)c4ccc(C5CC5)cc4F)[C@H]3C)cnc2C(N)=O)sn1]

In a similar manner as described in Example D-170, N-[(2R,3R)-1-{6-cyano-5-[(3-methyl-1,2-thiazol-5-yl)amino]pyridin-3-yl}-2-methylpiperidin-3-yl]-4-cyclopropyl-2-fluorobenzamide (D-116) was prepared. MS found for C26H27FN6OS as (M+H)⁺ 491.13.

To a solution of N-[(2R,3R)-1-{6-cyano-5-[(3-methyl-1,2-thiazol-5-yl)amino]pyridin-3-yl}-2-methylpiperidin-3-yl]-4-cyclopropyl-2-fluorobenzamide (91.6 mg, 0.186 mmol) in MeOH/DMSO (3/1 mL), NaOH (18 mg, 0.45 mmol). TEA (0.52 mL, 3.72 mmol) and H1O₂ 30% in water (0.08 mL) were added. The mixture was stirred at room temperature for 1 h then it was partitioned between ethyl acetate and water. The organic phase was dried over Na₂SO₄, concentrated and purified by silica flash chromatography, MeOH in DCM from 0 to 10% to give 5-[(2R,3R)-3-(4-cyclopropyl-2-fluorobenzamido)-2-methylpiperidin-1-yl]-3-[(3-methyl-1,2-thiazol-5-yl)amino]pyridine-2-carboxamide (D-116), (47.6 mg, 50% yield) as a yellow solid. MS found for C26H29FN6O2S as (M+H)⁺ 509.49.

¹H NMR (400 MHz, DMSO) δ 12.02 (s, 1H), 8.27 (d, J=7.03 Hz, 1H), 8.02 (d, J=2.26 Hz, 1H). 7.91 (d, J=2.51 Hz, 1H), 7.57 (d, J=2.26 Hz, 1H), 7.45 (t, J=7.91 Hz, 1H), 7.03-6.96 (m, 2H), 6.93 (d, J=2.26 Hz, 1H), 6.85 (s, 1H), 4.55-4.44 (m, 1H), 4.10-3.99 (m, 1H), 3.70 (d, J=11.54 Hz, 1H), 3.12-3.01 (m, 1H), 2.31 (s, 3H), 2.05-1.95 (m, 1H), 1.82 (d, J=10.54 Hz, 2H), 1.73-1.58 (m, 2H), 1.12 (d, J=6.78 Hz, 3H), 1.07-0.97 (m, 2H), 0.79-0.69 (m, 2H).

### Example D-117: Synthesis of 5-[(2R,3R)-3-(4-cyclopropyl-2-fluorobenzamido)-2-methylpiperidin-1-yl]-3-[(1-methyl-1H-pyrazol-4-yl)amino]pyridine-2-carboxamide (D-117)

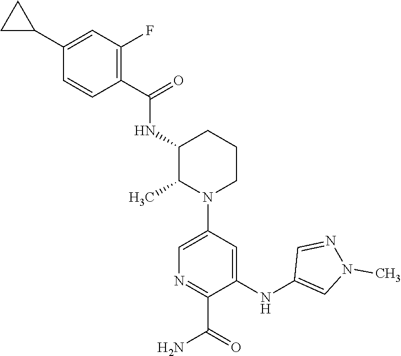 [SMILES: C[C@@H]1[C@H](NC(=O)c2ccc(C3CC3)cc2F)CCCN1c1cnc(C(N)=O)c(Nc2cnn(C)c2)c1]

In a similar manner as described in Example D-116, 5-[(2R,3R)-3-(4-cyclopropyl-2-fluorobenzamido)-2-methylpiperidin-1-yl]-3-[(1-methyl-1H-pyrazol-4-yl)amino]pyridine-2-carboxamide (D-117) was prepared. MS found for C26H30FN7O2 as (M+H)⁺ 492.51.

¹H NMR (400 MHz, DMSO) δ 9.96 (s, 1H), 8.21 (d, J=7.03 Hz, 1H), 7.83 (s, 1H), 7.76 (br. s., 1H), 7.66 (d, J=2.26 Hz, 1H), 7.51-7.40 (m, 2H), 7.21 (d, J=2.76 Hz, 1H), 7.07-6.93 (m, 2H), 6.62 (d, J=2.26 Hz, 1H), 4.41-4.33 (m, 1H), 4.09-3.95 (m, 1H), 3.81 (s, 3H), 3.51 (d, J=13.05 Hz, 1H), 3.00-2.90 (m, 1H), 2.05-1.94 (m, 1H), 1.88-1.47 (m, 4H), 1.09-0.97 (m, 5H), 0.81-0.71 (m, 2H).

### Example D-118: Synthesis of 5-[(2R,3R)-3-[(dimethylcarbamoyl)amino]-2-methylpiperidin-1-yl]-3-{[3-(morpholin-4-ylmethyl)-1,2-thiazol-5-yl]amino}pyrazine-2-carboxamide (D-118)

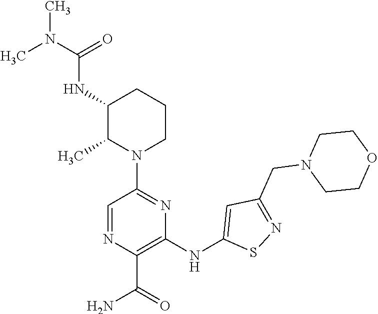 [SMILES: C[C@@H]1[C@H](NC(=O)N(C)C)CCCN1c1cnc(C(N)=O)c(Nc2cc(CN3CCOCC3)ns2)n1]

In a similar manner as described in Example 7, 5-[(2R,3R)-3-[(dimethylcarbamoyl)amino]-2-methylpiperidin-1-yl]-3-{[3-(morpholin-4-ylmethyl)-1,2-thiazol-5-yl]amino}pyrazine-2-carboxamide (D-118) was prepared using dimethylcarbamyl chloride. MS found for C22H33N903S as (M+H)⁺ 504.08.

¹H NMR (400 MHz, DMSO) δ 12.31 (s, 1H), 7.89 (br. s., 1H), 7.78 (s, 1H), 7.55 (s, 1H), 6.94 (s, 1H), 6.13 (d, J=6.80 Hz, 1H), 5.01-4.66 (m, 1H), 4.61-4.23 (m, 1H), 3.79-3.65 (m, 1H), 3.61-3.53 (m, 4H), 3.47 (d, J=1.97 Hz, 2H), 3.15-3.06 (m, 1H), 2.82 (s, 6H), 2.43-2.37 (m, 4H), 1.85 (d, J=12.06 Hz, 2H), 1.59 (br. s., 2H), 1.17 (d, J=7.02 Hz, 3H).

### Example D-119: Synthesis of 3-[(dimethyl-1,2-thiazol-5-yl)amino]-5-[(2R,3R)-3-[(dimethylcarbamoyl)amino]-2-methylpiperidin-1-yl]pyrazine-2-carboxamide (D-119)

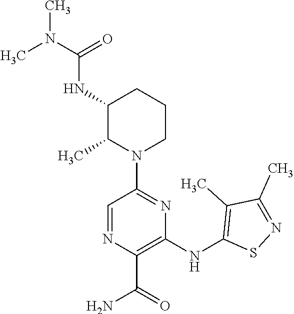 [SMILES: Cc1nsc(Nc2nc(N3CCC[C@@H](NC(=O)N(C)C)[C@H]3C)cnc2C(N)=O)c1C]

In a similar manner as described in Example 7, 3-[(dimethyl-1,2-thiazol-5-yl)amino]-5-[(2R,3R)-3-[(dimethylcarbamoyl)amino]-2-methylpiperidin-1-yl]pyrazine-2-carboxamide (D-119) was prepared using dimethylcarbamyl chloride. MS found for C19H28N8O2S as (M+H)⁺ 433.05.

¹H NMR (400 MHz, DMSO) δ 12.27 (s, 1H), 7.90 (br. s., 1H), 7.75 (s, 1H), 7.53 (br.s., 1H), 6.12 (d, J=7.15 Hz, 1H), 4.83 (br. s., 1H), 4.47 (br. s., 1H), 3.79-3.67 (m, 1H), 3.16-305 (m, 1H), 2.82 (s, 6H), 2.27 (s, 3H), 2.12 (s, 3H), 1.93-1.74 (m, 2H), 1.67-1.51 (m, 2H), 1.17 (d, J=6.90 Hz, 3H).

### Example D-120: Synthesis of 5-[(2R,3R)-3-[(dimethylcarbamoyl)amino]-2-methylpiperidin-1-yl]-3-((4-[(4-methylpiperazin-1-yl)methyl]phenyl) amino)pyridine-2-carboxamide (D-120)

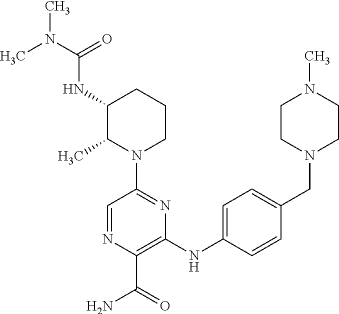 [SMILES: C[C@@H]1[C@H](NC(=O)N(C)C)CCCN1c1cnc(C(N)=O)c(Nc2ccc(CN3CCN(C)CC3)cc2)n1]

In a similar manner as described in Example 7, 5-[(2R,3R)-3-[(dimethylcarbamoyl)amino]-2-methylpiperidin-1-yl]-3-({4-[(4-methylpiperazin-1-yl)methyl]phenyl}amino)pyridine-2-carboxamide (D-120) was prepared using dimethylcarbamyl chloride. MS found for C27H40N8O2 as (M+H)⁺ 509.54.

¹H NMR (500 MHz, DMSO) δ 10.50 (s, 1H), 7.84 (d, J=2.45 Hz, 1H), 7.72 (d, J=2.20 Hz, 1H), 7.29 (d, J=2.50 Hz, 1H), 7.25 (d, J=8.31 Hz, 2H), 7.17 (d, J=8.31 Hz, 2H), 6.88 (d, J=2.20 Hz, 1H), 6.03 (d, J=6.85 Hz, 1H), 4.28-4.17 (m, 1H), 3.71 (d, J=4.65 Hz, 1H), 3.55-336 (m, 3H), 2.98-2.85 (m, 1H), 2.80 (s, 6H), 2.62-2.08 (m, 11H), 1.80-1.66 (m, 2H), 1.61-1.46 (m, 2H), 0.97 (d, J=6.85 Hz, 3H).

### Example D-121: Synthesis of 5-[(2R,3R)-3-[(dimethylcarbamoyl)amino]-2-methylpiperidin-1-yl]-3-[(quinolin-7-yl)amino]pyrazine-2-carboxamide (D-121)

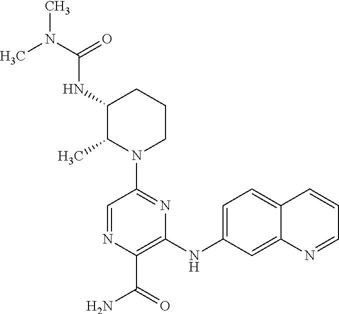 [SMILES: C[C@@H]1[C@H](NC(=O)N(C)C)CCCN1c1cnc(C(N)=O)c(Nc2ccc3cccnc3c2)n1]

In a similar manner as described in Example 7, 5-[(2R,3R)-3-[(dimethylcarbamoyl)amino]-2-methylpiperidin-1-yl]-3-[(quinolin-7-yl)amino]pyrazine-2-carboxamide (D-121) was prepared using dimethylcarbamyl chloride. MS found for C23H28N8O2 as (M+H)⁺ 449.43.

¹H NMR (500 MHz, DMSO) δ 11.75 (br. s., 1H), 8.80 (br. s., 1H), 8.42 (br. s., 1H), 8.31-8.13 (m, 1H), 7.96-7.82 (m, 2H), 7.73 (s, 2H), 7.50-7.43 (m, 1H), 7.39-7.32 (m, 1H), 6.14 (d, J=6.36 Hz, 1H), 4.83 (br. s., 1H), 4.38 (br. s., 1H), 3.74 (d, J=4.40 Hz, 1H), 3.09 (t, J=11.98 Hz, 1H), 2.83 (s, 6H), 1.92-1.44 (m, 4H), 1.17 (d, J=6.85 Hz, 3H).

### Example D-122: Synthesis of 5-[(2R,3R)-3-(4-cyclopropyl-2-fluorobenzamido)-2-methylpiperidin-1-yl]-3-({3-[(dimethylamino)methyl]-1,2-thiazol-5-yl}amino)pyrazine-2-carboxamide (D-122)

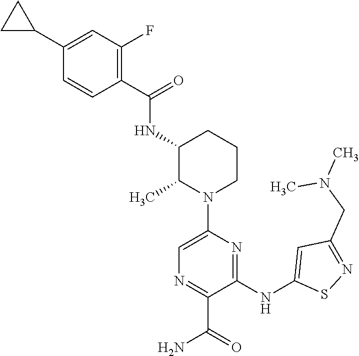 [SMILES: C[C@@H]1[C@H](NC(=O)c2ccc(C3CC3)cc2F)CCCN1c1cnc(C(N)=O)c(Nc2cc(CN(C)C)ns2)n1]

In a similar manner as described in Example D-216, 5-[(2R,3R)-3-(4-cyclopropyl-2-fluorobenzamido)-2-methylpiperidin-1-yl]-3-({3-[(dimethylamino)methyl]-1,2-thiazol-5-yl}amino)pyrazine-2-carboxamide (D-122) was prepared using 4-cyclopropyl-2-fluorobenzoic acid. MS found for C27H33FN8O2S as (M+H)⁺ 553.13.

¹H NMR (500 MHz, DMSO) δ 12.34 (s, 1H), 8.32 (d, J=7.41 Hz, 1H), 7.91 (br. s., 1H), 7.83 (s, 1H), 7.56 (br. s., 1H), 7.46 (t, J=7.89 Hz, 1H), 7.05-6.96 (m, 2H), 6.91 (s, 1H), 5.13 (br. s., 1H), 4.40 (br. s., 1H), 4.13-3.96 (m, 1H), 3.43-3.35 (m, 2H), 3.15 (t, J=12.28 Hz, 1H), 2.16 (s, 6H), 2.05-1.96 (m, 1H), 1.94-1.82 (m, 2H), 1.77-1.57 (m, 2H), 1.23 (d, J=6.86 Hz, 3H), 1.06-1.00 (m, 2H), 0.79-0.73 (m, 2H).

### Example D-123: Synthesis of 5-[(2R,3R)-3-(4-cyclopropyl-2-fluorobenzamido)-2-methylpiperidin-1-yl]-3-{[1-(propan-2-yl)-1H-pyrazol-4-yl]amino}pyrazine-2-carboxamide (D-123)

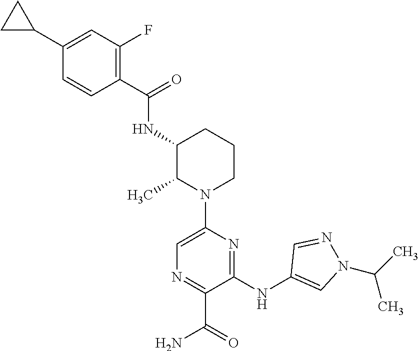 [SMILES: CC(C)n1cc(Nc2nc(N3CCC[C@@H](NC(=O)c4ccc(C5CC5)cc4F)[C@H]3C)cnc2C(N)=O)cn1]

In a similar manner as described in Example D-216, 5-[(2R,3R)-3-(4-cyclopropyl-2-fluorobenzamido)-2-methylpiperidin-1-yl]-3-{[1-(propan-2-yl)-1H-pyrazol-4-yl]amino}pyrazine-2-carboxamide (D-123) was prepared using 4-cyclopropyl-2-fluorobenzoic acid. MS found for C27H33FN8O2 as (M+H)⁺ 521.15.

¹H NMR (500 MHz, DMSO) δ 10.88 (s, 1H), 8.32 (d, J=6.86 Hz, 1H), 7.99 (s, 1H), 7.69 (br. s., 1H), 7.58 (s, 1H), 7.51-7.39 (m, 2H), 6.91 (s, 1H), 7.28 (br. s., 1H), 7.08-6.94 (m, 2H), 5.19 (br. s., 1H), 4.38 (quin, J=6.72 Hz, 1H), 4.12 (br. s., 1H), 4.07-3.98 (m, 1H), 3.13-3.00 (m, 1H), 2.07-1.96 (m, 1H), 1.93-1.78 (m, 2H), 1.74-1.53 (m, 2H), 1.26 (d, J=6.59 Hz, 3H), 1.20 (d, J=6.31 Hz, 3H), 1.15 (d, J=7.14 Hz, 3H), 1.06-0.99 (m, 2H), 0.80-0.71 (m, 2H).

### Example D-124: Synthesis of 5-[(2R,3R)-3-(4-cyclopropyl-2-fluorobenzamido)-2-methylpiperidin-1-yl]-3-[(quinolin-7-yl)amino]pyrazine-2-carboxamide (D-124)

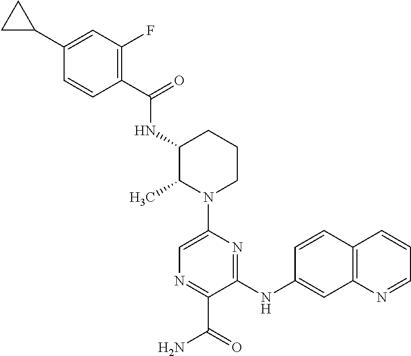 [SMILES: C[C@@H]1[C@H](NC(=O)c2ccc(C3CC3)cc2F)CCCN1c1cnc(C(N)=O)c(Nc2ccc3cccnc3c2)n1]

In a similar manner as described in Example D-216, 5-[(2R,3R)-3-(4-cyclopropyl-2-fluorobenzamido)-2-methylpiperidin-1-yl]-3-[(quinolin-7-yl)amino]pyrazine-2-carboxamide (D-124) was prepared using 4-cyclopropyl-2-fluorobenzoic acid. MS found for C30H30FN7O2 as (M+H)⁺ 540.47.

¹H NMR (400 MHz, DMSO) δ 12.77 (s, 1H), 8.72 (d, J=2.41 Hz, 1H), 8.45 (s, 1H), 8.32 (d, J=7.24 Hz, 1H), 8.20 (d, J=7.23 Hz, 1H), 7.88 (d, J=8.77 Hz, 2H), 7.78 (s, 1H), 7.69 (d, J=8.11 Hz, 1H), 7.54-7.43 (m, 2H), 7.34 (dd, J=8.11, 4.38 Hz, 1H), 7.09-6.94 (m, 2H), 5.04 (br. s., 1H), 4.36 (br. s., 1H), 4.16-3.95 (m, 1H), 3.22-3.02 (m, 1H), 2.08-1.97 (m, 1H), 1.95-1.79 (m, 2H), 1.76-1.58 (m, 2H), 1.25 (d, J=6.80 Hz, 3H), 1.10-0.98 (m, 2H), 0.87-0.70 (m, 2H).

### Example D-125: Synthesis of 5-[(2R,3R)-3-(4-cyclopropyl-2-fluorobenzamido)-2-methylpiperidin-1-yl]-3-(4-[(4-methylpiperazin-1-yl)methyl]phenyl amino)pyridine-2-carboxamide (D-125)

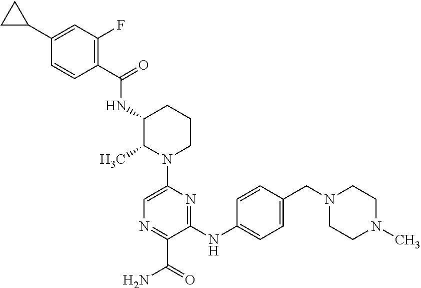 [SMILES: C[C@@H]1[C@H](NC(=O)c2ccc(C3CC3)cc2F)CCCN1c1cnc(C(N)=O)c(Nc2ccc(CN3CCN(C)CC3)cc2)n1]

In a similar manner as described in Example D-216, 5-[(2R,3R)-3-(4-cyclopropyl-2-fluorobenzamido)-2-methylpiperidin-1-yl]-3-({4-[(4-methylpiperazin-1-yl)methyl]phenyl}amino)pyridine-2-carboxamide (D-125) was prepared using 4-cyclopropyl-2-fluorobenzoic acid. MS found for C34H42FN7O2 as (M+H)⁺ 600.53.

¹H NMR (400 MHz, DMSO) δ 10.49 (s, 1H), 8.19 (d, J=6.80 Hz, 1H), 7.85 (d, J=2.63 Hz, 1H), 7.75 (d, J=2.41 Hz, 1H), 7.46-7.39 (m, 1H), 7.35-7.29 (m, 1H), 7.27-7.22 (m, 2H), 7.21-7.15 (m, 2H), 7.04-6.88 (m, 3H), 4.39-4.28 (m, 1H), 4.10-3.96 (m, 1H), 3.59-3.47 (m, 1H), 3.39 (s, 2H), 3.01-2.89 (m, 1H), 2.44-2.16 (m, 8H), 2.11 (s, 3H), 2.03-1.93 (m, 1H), 1.86-1.50 (m, 4H), 1.08-0.98 (m, 5H), 0.79-0.69 (m, 2H).

### Example D-126: Synthesis of 5-[(2R,3R)-3-(4-cyclopropyl-2-fluorobenzamido)-2-methylpiperidin-1-yl]-3-{[4-(4-methylpiperazin-1-yl)phenyl]amino}pyridine-2-carboxamide (D-126)

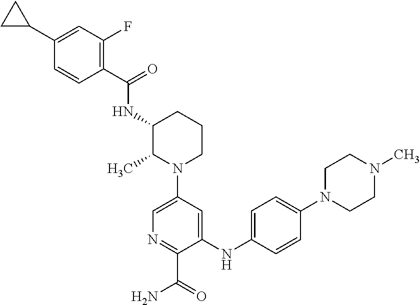 [SMILES: C[C@@H]1[C@H](NC(=O)c2ccc(C3CC3)cc2F)CCCN1c1cnc(C(N)=O)c(Nc2ccc(N3CCN(C)CC3)cc2)c1]

In a similar manner as described in Example D-116, 5-[(2R,3R)-3-(4-cyclopropyl-2-fluorobenzamido)-2-methylpiperidin-1-yl]-3-([4-(4-methylpiperazin-1-yl)phenyl]amino)pyridine-2-carboxamide (D-126) was prepared. MS found for C33H40FN7O2 as (M+H)⁺ 586.52.

¹H NMR (500 MHz, DMSO) δ 10.17 (s, 1H), 8.18 (d, J=6.86 Hz, 1H), 7.79 (d, J=2.74 Hz, 1H), 7.67 (d, J=2.47 Hz, 1H), 7.47-7.39 (m, 1H), 7.23 (d, J=3.29 Hz, 1H), 7.09 (d, J=8.78 Hz, 2H), 7.01-6.94 (m, 2H), 6.92 (d, J=9.06 Hz, 2H), 6.69-6.66 (m, 1H), 4.35-4.22 (m, 1H), 4.10-3.93 (m, 1H), 3.45-3.36 (m, 1H), 3.12-3.01 (m, 4H), 2.94-2.83 (m, 1H), 2.45-2.38 (m, 4H), 2.20 (s, 3H), 2.04-1.93 (m, 1H), 1.82-1.49 (m, 4H), 1.07-0.95 (m, 5H), 0.77-0.70 (m, 2H).

### Example D-127: Synthesis of 5-[(2R,3R)-3-[(dimethylcarbamoyl)amino]-2-methylpiperidin-1-yl]-3-{[4-(4-methylpiperazin-1-yl)phenyl]amino}pyridine-2-carboxamide (D-127)

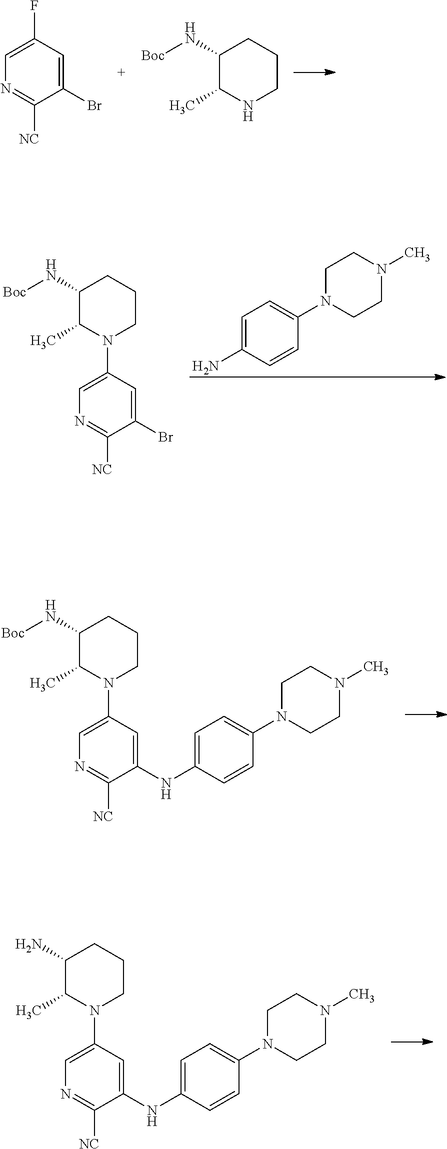

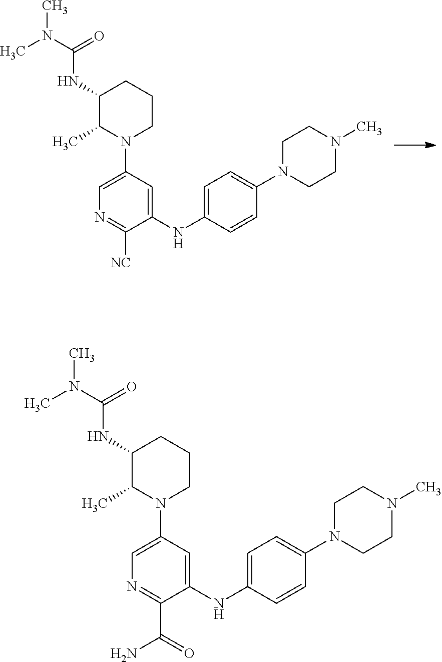

3-bromo-5-fluoropyridine-2-carbonitrile (470.0 mg, 2.33 mmol), tert-butyl N-[(2R,3R)-2-methylpiperidin-3-yl]carbamate (500.0 mg. 2.33 mmol) and DIPEA (820.0 μL, 4.66 mmol) were dissolved in DMF (8 mL). The mixture was stirred at room temperature overnight. 3-Bromo-5-fluoropyridine-2-carbonitrile (50.0 mg, 0.25 mmol) was added and the reaction stirred overnight at 90° C. DMF was evaporated and the product purified by silica flash chromatography with 0 to 100% ethyl acetate in cyclohexane to give tert-butyl N-[(2R,3R)-1-(5-bromo-6-cyanopyridin-3-yl)-2-methylpiperidin-3-yl]carbamate (869.0 mg, 95% yield). MS found for C17H23BrN4O2 as (M+H)⁺ 395.3, 397.3.

Tert-butyl N-[(2R,3R)-1-(5-bromo-6-cyanopyridin-3-yl)-2-methylpiperidin-3-yl]carbamate (200.0 mg, 0.50 mmol) were suspended in dioxane (10 mL), 4-(4-methylpiperazin-1-yl)aniline (145.5 mg, 0.76 mmol). Cs₂CO₃ (6580.0 mg, 2.02 mmol). (+/−) BINAP (70.0 mg, 0.11) and Pd(OAc)₂ (25.0 mg, 0.11 mmol) were added while degassing with nitrogen. The mixture was heated at 110° C. for 2 h. The solvent was evaporated and the residue purified by silica flash chromatography with cyclohexane:ethyl acetate from 0 to 100% and then 0 to 20% MeOH in ethyl acetate to give tert-butyl N-[(2R,3R)-1-(6-cyano-5-{[4-(4-methylpiperazin-1-yl)phenyl]amino}pyridin-3-yl)-2-methylpiperidin-3-yl]carbamate (238.3 mg, 93% yield). MS found for C28H39N7O2 as (M+H)⁺ 506.53.

Tert-butyl N-[(2R,3R)-1-(6-cyano-5-{[4-(4-methylpiperazin-1-yl)phenyl]amino}pyridin-3-yl)-2-methylpiperidin-3-yl]carbamate (238.3 mg, 0.47 mmol) was dissolved in HCl in MeOH 1.25 N (3.0 ml, 3.77 mmol) and stirred at room temperature overnight. The solvent was evaporated to give a solid which was passed through an SCX cartridge. Evaporation of the ammonia fractions gave 5-[(2R,3R)-3-amino-2-methylpiperidin-1-yl]-3-{[4-(4-methylpiperazin-1-yl)phenyl]amino}pyridine-2-carbonitrile (171.2 mg, 89% yield). MS found for C23H31N7 as (M+H)⁺ 406.54.

To a solution of 5-[(2R,3R)-3-amino-2-methylpiperidin-1-yl]-3-{[4-(4-methylpiperazin-1-yl)phenyl]amino}pyridine-2-carbonitrile (85.6 mg, 0.21 mmol) in DMF (3 ml) DIPEA (0.110 mL, 0.633 mmol) dimethylcarbamyl chloride (24.98 mg, 0.23 mmol) were added. The mixture was stirred at room temperature overnight then concentrated in vacuo. The residue was purified by silica flash chromatography with 0 to 25% MeOH in DCM to afford 1-[(2R,3R)-1-(6-cyano-5-{[4-(4-methylpiperazin-1-yl)phenyl]amino}pyridin-3-yl)-2-methylpiperidin-3-yl]-3,3-dimethylurea (99.0 mg, 98% yield). MS found for C26H36N8O as (M+H)⁺ 477.51.

To a solution of 1-[(2R,3R)-1-(6-cyano-5-{[4-(4-methylpiperazin-1-yl)phenyl]amino}pyridin-3-yl)-2-methylpiperidin-3-yl]-3,3-dimethylurea (99.0 mg, 0.207 mmol) in MeOH/DMSO (6/2 mL), NaOH (20 mg, 0.498 mmol), TEA (0.58 mL, 4.14 mmol) and H₂O₂ 30% in water (0.15 mL) were added. The mixture was stirred at room temperature for 1 h then concentrated in vacuo and after it was partitioned between ethyl acetate and water. The organic phase was dried over Na₂SO₄, concentrated and purified by flash chromatography silica, cyclohexane:ethyl acetate from 50 to 100 and then with 0 to 20% MeOH in ethyl acetate to give 5-[(2R,3R)-3-[(dimethylcarbamoyl)amino]-2-methylpiperidin-1-yl]-3-{[4-(4-methylpiperazin-1-yl)phenyl]amino}pyridine-2-carboxamide (D-127, 50.9 mg, 49% yield) as a yellow solid. MS found for C₂₆H₃₈N₈O₂ as (M+H)⁺ 495.52.

¹H NMR (500 MHz, DMSO) δ 10.09-10.28 (m, 1H), 7.77 (d, J=3.02 Hz, 1H), 7.64 (d, J=2.47 Hz, 1H), 7.21 (d, J=3.02 Hz, 1H), 7.08 (d, J=9.06 Hz, 2H), 6.97-6.88 (m, 2H), 6.65 (d, J=2.20 Hz, 1H), 6.00 (d, J=6.86 Hz, 1H), 4.23-4.13 (m, 1H), 3.76-3.64 (m, 1H), 3.43-3.34 (m, 1H), 3.12-3.04 (m, 4H), 2.89-2.81 (m, 1H), 2.78 (s, 6H), 2.46-2.40 (m, 4H), 2.21 (s, 3H), 1.78-1.40 (m, 4H), 0.93 (d, J=6.86 Hz, 3H).

### Example D-128: Synthesis of 5-[(2R,3R)-3-[(dimethylcarbamoyl)amino]-2-methylpiperidin-1-yl]-3-[(1-methyl-1H-pyrazol-4-yl)amino]pyrazine-2-carboxamide (D-128)

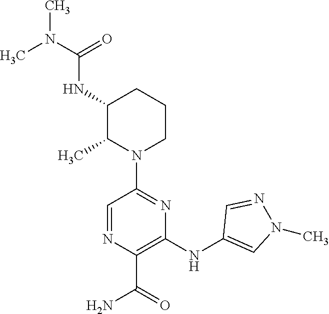

In a similar manner as described in Example 7, 5-[(2R,3R)-3-[(dimethylcarbamoyl)amino]-2-methylpiperidin-1-yl]-3-[(1-methyl-1H-pyrazol-4-yl)amino]pyrazine-2-carboxamide (D-128) was prepared using dimethylcarbamyl chloride. MS found for C18H27N9O2 as (M+H)⁺ 402.01.

¹H NMR (400 MHz, DMSO) δ 10.85 (s, 1H), 8.09-7.96 (m, 1H), 7.73-7.61 (m, 1H), 7.52 (s, 2H), 7.49-7.42 (m, 1H), 7.30-7.22 (m, 1H), 6.10 (d, J=6.59 Hz, 1H), 5.53-4.93 (m, 1H), 4.17-3.97 (m, 1H), 3.84 (s, 3H), 3.75-3.60 (m, 1H), 3.07-2.97 (m, 1H), 2.84 (s, 6H), 1.88-1.44 (m, 4H), 1.03 (d, J=6.86 Hz, 3H).

### Example D-129: Synthesis of 5-[(2R,3R)-3-(4-cyclopropyl-2-fluorobenzamido)-2-methylpiperidin-1-yl]-3-[(1-methyl-1H-pyrazol-3-yl)amino]pyrazine-2-carboxamide (D-129)

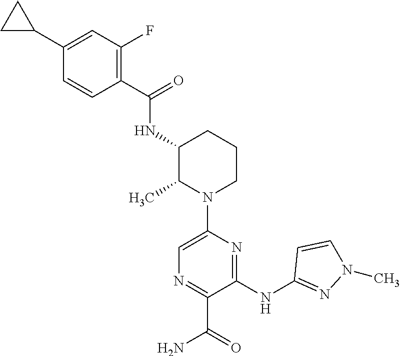 [SMILES: C[C@@H]1[C@H](NC(=O)c2ccc(C3CC3)cc2F)CCCN1c1cnc(C(N)=O)c(Nc2ccn(C)n2)n1]

In a similar manner as described in Example 65, 5-[(2R,3R)-3-(4-cyclopropyl-2-fluorobenzamido)-2-methylpiperidin-1-yl]-3-[(1-methyl-1H-pyrazol-3-yl)amino]pyrazine-2-carboxamide (D-129) was prepared using N-[(2R,3R)-1-(6-chloro-5-cyanopyrazin-2-yl)-2-methylpiperidin-3-yl]-4-cyclopropyl-2-fluorobenzamide. MS found for C25H29FN8O2 as (M+H)⁺ 492.22.

¹H NMR (500 MHz, DMSO) δ 11.32 (s, 1H), 8.28 (d, J=7.41 Hz, 1H), 7.73 (br. s., 1H), 7.63 (s, 1H), 7.49 (d, J=2.20 Hz, 1H), 7.47-7.42 (m, 1H), 7.33 (d, J=1.92 Hz, 1H), 7.04-6.96 (m, 2H), 6.56 (d, J=2.20 Hz, 1H), 5.06 (br. s., 1H), 4.20 (br. s., 1H), 4.06-3.94 (m, 1H), 3.72 (s, 3H), 3.09-2.98 (m, 1H), 2.09-1.95 (m, 1H), 1.91-1.77 (m, 2H), 1.73-1.52 (m, 2H), 1.13 (d, J=6.86 Hz, 3H), 1.06-0.99 (m, 2H), 0.82-0.70 (m, 2H).

### Example D-130: Synthesis of 5-[(2R,3R)-3-(4-cyclopropyl-2-fluorobenzamido)-2-methylpiperidin-1-yl]-3-({4-[(4-methylpiperazin-1-yl)sulfonyl]phenyl}amino)pyrazine-2-carboxamide (D-130)

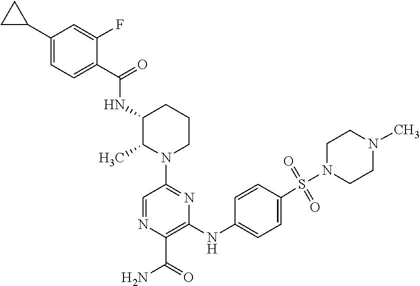 [SMILES: C[C@@H]1[C@H](NC(=O)c2ccc(C3CC3)cc2F)CCCN1c1cnc(C(N)=O)c(Nc2ccc(S(=O)(=O)N3CCN(C)CC3)cc2)n1]

In a similar manner as described in Example 65, 5-[(2R,3R)-3-(4-cyclopropyl-2-fluorobenzamido)-2-methylpiperidin-1-yl]-3-({4-[(4-methylpiperazin-1-yl)sulfonyl]phenyl}amino)pyrazine-2-carboxamide (D-130) was prepared using N-[(2R,3R)-1-(6-chloro-5-cyanopyrazin-2-yl)-2-methylpiperidin-3-yl]-4-cyclopropyl-2-fluorobenzamide. MS found for C32H39FN8O4S as (M+H)⁺ 651.20.

¹H NMR (500 MHz, DMSO) δ 11.77 (s, 1H), 8.35 (d, J=7.14 Hz, 1H), 7.93-7.85 (m, 3H), 7.79 (s, 1H), 7.63 (d, J=8.78 Hz, 1H), 7.53-7.44 (m, 2H), 7.06-6.96 (m, 2H), 5.30 (br. s., 1H), 4.13 (br. s., 1H), 4.08-3.99 (m, 1H), 3.12 (t, J=12.60 Hz, 1H), 2.74 (br. s., 1H), 2.24 (br. s., 1H), 2.09 (s, 3H), 2.05-1.98 (m, 1H), 1.92-1.80 (m, 2H), 1.72-1.55 (m, 2H), 1.13 (d, J=6.86 Hz, 3H), 1.07-1.00 (m, 2H), 0.80-0.70 (m, 2H).

### Example D-131: Synthesis of 3-[(3-methyl-1,2-thiazol-5-yl)amino]-5-[(2R,3R)-2-methyl-3-[5-(4-methylphenyl)-1H-imidazol-2-yl]piperidin-1-yl]pyrazine-2-carboxamide (D-131)

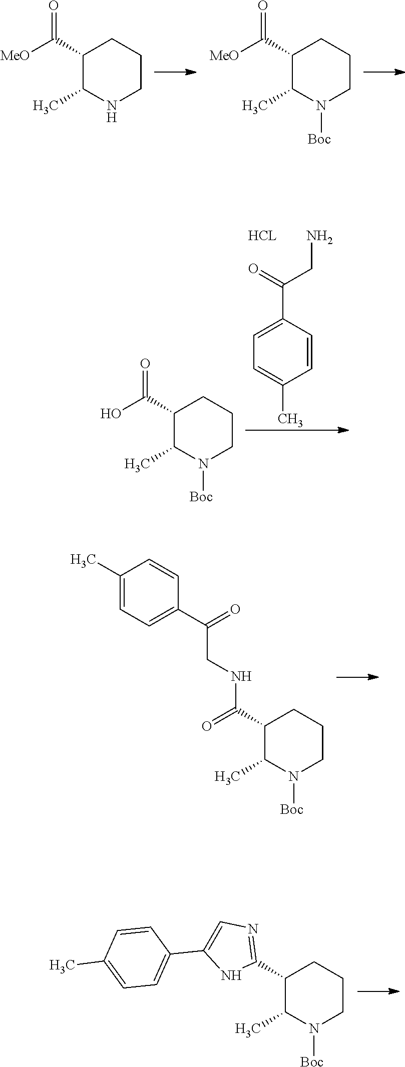

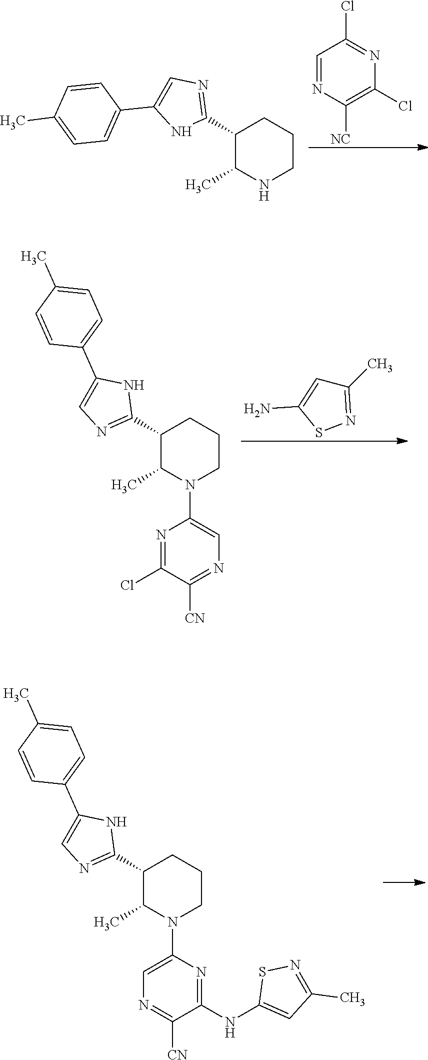

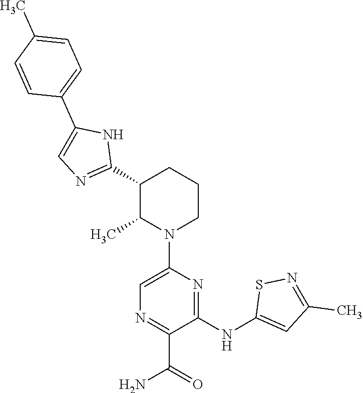 [SMILES: Cc1ccc(-c2cnc([C@@H]3CCCN(c4cnc(C(N)=O)c(Nc5cc(C)ns5)n4)[C@@H]3C)[nH]2)cc1]

Methyl (2R,3R)-2-methylpiperidine-3-carboxylate (4 g, 25.0 mmol) were dissolved in THF 50% aqueous (75 mL) and sodium carbonate (2.72 g, 25.6 mmol) were added. The solution was stirred at 0° C., di-tert-butyl dicarbonate (6.24 g, 28.6 mmol) and sodium carbonate (3.2 g, 30.2 mmol) dissolved in THF 50% aqueous (60 mL) was added at 0° C. and then left at room temperature overnight. The mixture was neutralized with HCl 6 N and then extracted with DCM. The organic phase was separated, dried over Na₂SO₄ and concentrated under reduced pressure to afford 1-tert-butyl 3-methyl (2R,3R)-2-methylpiperidine-1,3-dicarboxylate (6.32 g, 98% yield) as a white solid. MS found for C13H23NO4 as (M+H)⁺ 258.34.

1-Tert-butyl 3-methyl (2R,3R)-2-methylpiperidine-1,3-dicarboxylate (6.32 g, 24.57 mmol) was dissolved in THF (60 mL) and LiOH 2 M (61.5 mL, 122.85 mmol) were added and the mixture stirred at room temperature overnight. The mixture was concentrated under reduced pressure, dissolved in water (100 mL), neutralized with HCl 1N and extracted with ethyl acetate. The organic phase washed with water, brine, separated, dried over Na₂SO₄ and concentrated to give (2R,3R)-1-[(teroom temperature-butoxy)carbonyl]-2-methylpiperidine-3-carboxylic acid (5.95 g, 99% yield) as a white solid. MS found for C12H21NO4 as (M+H)⁺ 275.12.

(2R,3R)-1-[(Tert-butoxy)carbonyl]-2-methylpiperidine-3-carboxylic acid (817.55 mg, 1.25 mmol) was dissolved in THF (25 mL), cooled at −10° C. and N-methylmorpholine (0.89 mL, 8.07 mmol) was added. After 5 minutes isobutyl chloroformate (0.35 mL, 2.69 mmol) was added and the mixture was stirred in these conditions for 2 h. Then 2-amino-1-(4-methylphenyl)ethan-1-one hydrochloride (500.0 mg, 2.69 mmol) was added and the mixture was stirred at room temperature overnight. To the mixture was added DCM, the insoluble material was filtered off and the filtrate was washed with NaHCO₃ sat. aqueous solution. The organic phase was separated, dried over Na₂SO₄ and concentrated. The residue was purified by silica flash chromatography with 0 to 100% ethyl acetate in cyclohexane to give tert-butyl (2R,3R)-2-methyl-3-{[2-(4-methylphenyl)-2-oxoethyl]carbamoyl}piperidine-1-carboxylate (538.5 mg, 53% yield). MS found for C21H30N2O4 as (M+H)⁺ 375.43.

Tert-butyl (2R,3R)-2-methyl-3-{[2-(4-methylphenyl)-2-oxoethyl]carbamoyl}piperidine-1-carboxylate (538.5 mg, 1.44 mmol) was dissolved in 1-butanol (5 mL), TEA (200.0 μL, 1.43 mmol) and ammonium acetate (3.3 g, 43.14 mmol) were added and the mixture was heated at 150° C. for 3 h. The mixture was concentrated; the residue was redissolved in ethyl acetate and washed with water. The organic phase was separated, dried over Na₂SO₄ and concentrated. The residue purified by silica flash chromatography with 0 to 100% ethyl acetate in cyclohexane to afford tert-butyl (2R,3R)-2-methyl-3-[5-(4-methylphenyl)-1H-imidazol-2-yl]piperidine-1-carboxylate (347.6 mg, 68% yield) as a yellow solid. MS found for C21H29N3O2 as (M+H)⁺ 356.44.

Tert-butyl (2R,3R)-2-methyl-3-[5-(4-methylphenyl)-1H-imidazol-2-yl]piperidine-1-carboxylate (347.6 mg, 0.978 mmol) was dissolved in DCM (9 mL), cooled at −10° C. and HCl 4 M in dioxane (4.5 mL, 18.78 mmol) were added and the mixture was stirred for 2 h. The solvent was evaporated to give a white solid which was passed through an SCX cartridge. Evaporation of the ammonia fractions gave (2R,3R)-2-methyl-3-[5-(4-methylphenyl)-1H-imidazol-2-yl]piperidine (250.0 mg, 100% yield). MS found for C16H21N3 as (M+H)⁺ 256.33.

3,5-Dichloropyrazine-2-carbonitrile (170.46 mg, 0.97 mmol) and (2R,3R)-2-methyl-3-[5-(4-methylphenyl)-1H-imidazol-2-yl]piperidine (250.0 mg, 0.97 mmol) were dissolved in DMF (3 mL), DIPEA (350.0 μL, 1.96 mmol) and the mixture was stirred at room temperature overnight. The mixture was poured into ice and extracted with ethyl acetate. The organic phase was separated, dried over Na₂SO₄ and concentrated. The residue purified by silica flash chromatography with 0 to 100% ethyl acetate in cyclohexane to give 3-chloro-5-[(2R,3R)-2-methyl-3-[5-(4-methylphenyl)-1H-imidazol-2-yl]piperidin-1-yl]pyrazine-2-carbonitrile (296.2 mg, 77% yield) as a white solid. MS found for C21H21ClN6 as (M+H)⁺ 393.39.

3-Chloro-5-[(2R,3R)-2-methyl-3-[5-(4-methylphenyl)-1H-imidazol-2-yl]piperidin-1-yl]pyrazine-2-carbonitrile (100.0 mg, 0.255 mmol), 3-methyl-1,2-thiazol-5-amine hydrochloride (57.61 mg, 0.38 mmol), and Cs₂CO₃ (350.0 mg, 1.07 mmol) were suspended in dioxane (5 mL). (+/−) BINAP (32 mg, 0.051 mmol) and Pd(OAc)₂ (14.0 mg, 0.051 mmol) were added under nitrogen and the mixture stirred for 2 h at 120° C. The insoluble material was filtered off and the filtrate concentrated. The residue purified by silica flash chromatography with 0 to 100% ethyl acetate in cyclohexane to give 3-[(3-methyl-1,2-thiazol-5-yl)amino]-5-[(2R,3R)-2-methyl-3-[5-(4-methylphenyl)-1H-imidazol-2-yl]piperidin-1-yl]pyrazine-2-carbonitrile (72.3 mg, 60% yield). MS found for C25H26N8S as (M+H)⁺ 471.50.

To a suspension of 3-[(3-methyl-1,2-thiazol-5-yl)amino]-5-[(2R,3R)-2-methyl-3-[5-(4-methylphenyl)-1H-imidazol-2-yl]piperidin-1-yl]pyrazine-2-carbonitrile (72.3 mg, 0.154 mmol) in MeOH/DMSO (6 mL/2 mL), NaOH (14.76 mg, 0.37 mmol), TEA (0.45 mL, 3.09 mmol) and H₂O₂ (0.45 mL) were added. The mixture was stirred overnight at room temperature, then it was partitioned between DCM and H₂O. The combined organic phase were dried over Na₂SO₄ and concentrated. The crude was purified by silica flash chromatography with 50 to 100% ethyl acetate in cyclohexane to give 3-[(3-methyl-1,2-thiazol-5-yl)amino]-5-[(2R,3R)-2-methyl-3-[5-(4-methylphenyl)-1H-imidazol-2-yl]piperidin-1-yl]pyrazine-2-carboxamide (D-131) (53.0 mg, 70% yield) as a yellow solid. MS found for C25H28N8OS as (M+H)⁺ 489.11.

¹H NMR (400 MHz, DMSO) δ 12.30 (s, 1H), 12.07-11.83 (m, 1H), 7.96-7.84 (m, 2H), 7.78-7.48 (m, 2H), 7.59-7.47 (m, 2H), 7.26-7.09 (m, 2H), 6.85 (s, 1H), 5.83-4.04 (m, 2H), 3.29-3.13 (m, 2H), 2.35-2.27 (m, 6H), 2.26-2.11 (m, 1H), 2.07-1.88 (m, 2H), 1.77-1.58 (m, 1H), 1.06 (d, J=6.80 Hz, 3H).

### Example D-132: Synthesis of 5-[(2R,3R)-3-(4-cyclopropyl-2-fluorobenzamido)-2-methylpiperidin-1-yl]-3-({5-[(4-methylpiperazin-1-yl)methyl]pyridin-2-yl}amino)pyrazine-2-carboxamide (D-132)

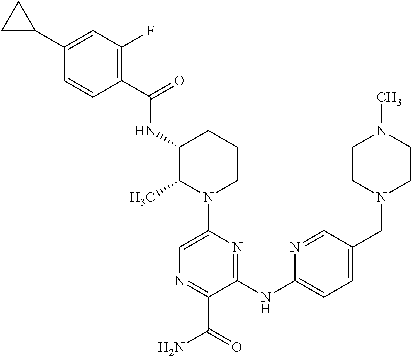 [SMILES: C[C@@H]1[C@H](NC(=O)c2ccc(C3CC3)cc2F)CCCN1c1cnc(C(N)=O)c(Nc2ccc(CN3CCN(C)CC3)cn2)n1]

In a similar manner as described in Example 65, 5-[(2R,3R)-3-(4-cyclopropyl-2-fluorobenzamido)-2-methylpiperidin-1-yl]-3-({5-[(4-methylpiperazin-1-yl)methyl]pyridin-2-yl}amino)pyrazine-2-carboxamide (D-132) was prepared using N-[(2R,3R)-1-(6-chloro-5-cyanopyrazin-2-yl)-2-methylpiperidin-3-yl]-4-cyclopropyl-2-fluorobenzamide. MS found for C32H40FN9O2 as (M+H)⁺ 602.23.

¹H NMR (500 MHz, DMSO) δ 11.69 (s, 1H), 8.32 (d, J=6.86 Hz, 1H), 8.26 (d, J=8.51 Hz, 1H), 8.12 (d, J=1.92 Hz, 1H), 7.82 (d, J=1.92 Hz, 1H), 7.75 (s, 1H), 7.62 (dd, J=8.51, 2.20 Hz, 1H), 7.47 (t, J=7.96 Hz, 1H), 7.43 (d, J=2.20 Hz, 1H), 7.06-6.96 (m, 2H), 5.24 (br. s., 1H), 4.15 (br. s., 1H), 4.02 (td, J=12.08, 4.67 Hz, 1H), 3.38 (s, 2H), 3.14-3.02 (m, 1H), 2.47-2.05 (m, 8H), 2.12 (s, 3H), 2.04-1.98 (m, 1H), 1.89-1.80 (m, 2H), 1.73-1.53 (m, 2H), 1.13 (d, J=6.86 Hz, 3H), 1.07-0.99 (m, 2H), 0.79-0.71 (m, 2H).

### Example D-133: Synthesis of 5-[(2R,3R)-3-(4-cyclopropyl-2-fluorobenzamido)-2-methylpiperidin-1-yl]-3-[(4-methanesulfonylphenyl)amino]pyrazine-2-carboxamide (D-133)

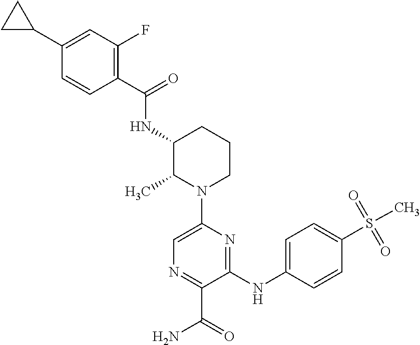 [SMILES: C[C@@H]1[C@H](NC(=O)c2ccc(C3CC3)cc2F)CCCN1c1cnc(C(N)=O)c(Nc2ccc(S(C)(=O)=O)cc2)n1]

In a similar manner as described in Example 65, 5-[(2R,3R)-3-(4-cyclopropyl-2-fluorobenzamido)-2-methylpiperidin-1-yl]-3-[(4-methanesulfonylphenyl)amino]pyrazine-2-carboxamide (D-133) was prepared using N-[(2R,3R)-1-(6-chloro-5-cyanopyrazin-2-yl)-2-methylpiperidin-3-yl]-4-cyclopropyl-2-fluorobenzamide. MS found for C28H31FN6O4S as (M+H)⁺ 567.16.

¹H NMR (500 MHz, DMSO) δ 11.83 (s, 1H), 8.30 (d, J=7.02 Hz, 1H), 7.96-7.75 (m, 6H), 7.57-7.46 (m, 2H), 7.08-6.93 (m, 2H), 5.22 (br. s., 1H), 4.19 (d, J=1.07 Hz, 1H), 4.08-3.97 (m, 1H), 3.18-3.05 (m, 4H), 2.07-1.96 (m, 1H), 1.95-1.78 (m, 2H), 1.77-1.54 (m, 2H), 1.13 (d, J=6.91 Hz, 3H), 1.07-0.98 (m, 2H), 0.81-0.71 (m, 2H).

### Example D-134: Synthesis of 3-{[4-(1-cyclobutyl-4-methylpiperidin-4-yl)phenyl]amino}-5-[(2R,3R)-3-(4-cyclopropyl-2-fluorobenzamido)-2-methylpiperidin-1-yl]pyrazine-2-carboxamide (D-134)

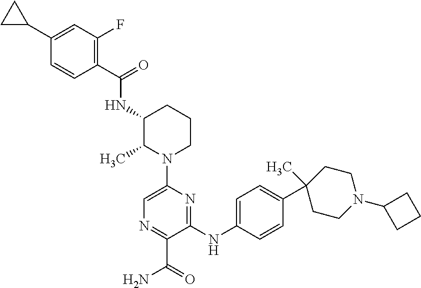 [SMILES: C[C@@H]1[C@H](NC(=O)c2ccc(C3CC3)cc2F)CCCN1c1cnc(C(N)=O)c(Nc2ccc(C3(C)CCN(C4CCC4)CC3)cc2)n1]

In a similar manner as described in Example 65, 3-{[4-(1-cyclobutyl-4-methylpiperidin-4-yl)phenyl]amino}-5-[(2R,3R)-3-(4-cyclopropyl-2-fluorobenzamido)-2-methylpiperidin-1-yl]pyrazine-2-carboxamide (D-134) was prepared using N-[(2R,3R)-1-(6-chloro-5-cyanopyrazin-2-yl)-2-methylpiperidin-3-yl]-4-cyclopropyl-2-fluorobenzamide. MS found for C37H46FN7O2 as (M+H)⁺ 640.34.

¹H NMR (500 MHz, DMSO) δ 11.17 (s, 1H), 8.31 (d, J=7.68 Hz, 1H), 7.75 (br. s., 1H), 7.65 (s, 1H), 7.54 (d, J=8.65 Hz, 2H), 7.51-7.43 (m, 1H), 7.33 (d, J=2.06 Hz, 1H), 7.21 (d, J=8.60 Hz, 2H), 7.06-6.95 (m, 2H), 5.17 (br. s., 1H), 4.22-4.00 (m, 2H), 3.07 (t. J=12.08 Hz, 1H), 2.56 (quin, J=7.79 Hz, 1H), 2.24-1.95 (m, 5H), 1.94-1.44 (m, 14H), 1.12 (d, J=6.86 Hz, 3H), 1.07-1.00 (m, 5H), 0.78-0.71 (m, 2H).

### Example D-135: Synthesis of 5-[(2R,3R)-3-(4-cyclopropylbenzamido)-2-methylpiperidin-1-yl]-3-[(1-methyl-1H-pyrazol-4-yl)amino]pyrazine-2-carboxamide (D-135)

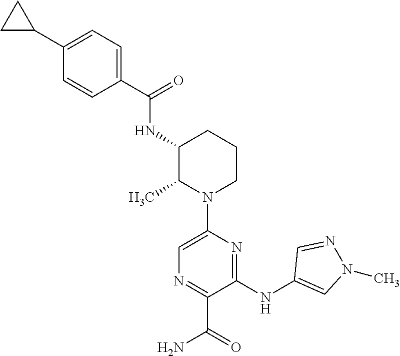 [SMILES: C[C@@H]1[C@H](NC(=O)c2ccc(C3CC3)cc2)CCCN1c1cnc(C(N)=O)c(Nc2cnn(C)c2)n1]

In a similar manner as described in Example D-216, 5-[(2R,3R)-3-(4-cyclopropylbenzamido)-2-methylpiperidin-1-yl]-3-[(1-methyl-1H-pyrazol-4-yl)amino]pyrazine-2-carboxamide (D-135) was prepared using 4-cyclopropylbenzoic acid. MS found for C25H30N8O2 as (M+H)⁺ 475.04.

¹H NMR (400 MHz, DMSO) δ 10.86 (s, 1H), 8.33 (d, J=6.65 Hz, 1H), 8.03 (s, 1H), 7.82 (d, J=1=8.61 Hz, 2H), 7.68 (br. s., 1H), 7.56 (s, 1H), 7.47 (s, 1H), 7.27 (d, J=1.57 Hz, 1H), 7.17 (d, J=8.61 Hz, 2H), 5.48-5.15 (m, 1H), 4.04 (br. s., 2H), 3.75 (s, 3H), 3.13-3.00 (m, 1H), 1.98 (d, J=4.70 Hz, 3H), 1.72 (d, J=2.35 Hz, 2H), 1.08 (d, J=6.65 Hz, 3H), 1.05-0.96 (m, 2H), 0.80-0.69 (m, 2H).

### Example D-136: Synthesis of 3-[(3-methyl-1,2-thiazol-5-yl)amino]-5-[(3S)-3-[5-(4-methylphenyl)-1H-imidazol-2-yl]piperidin-1-yl]pyrazine-2-carboxamide (D-136)

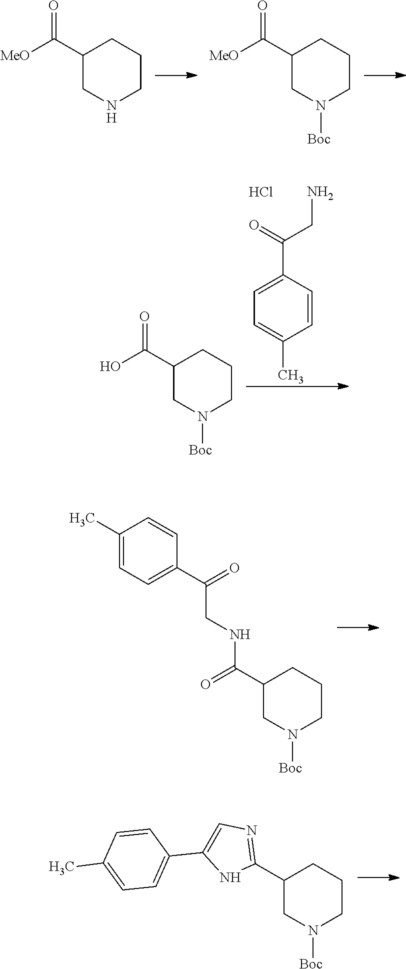

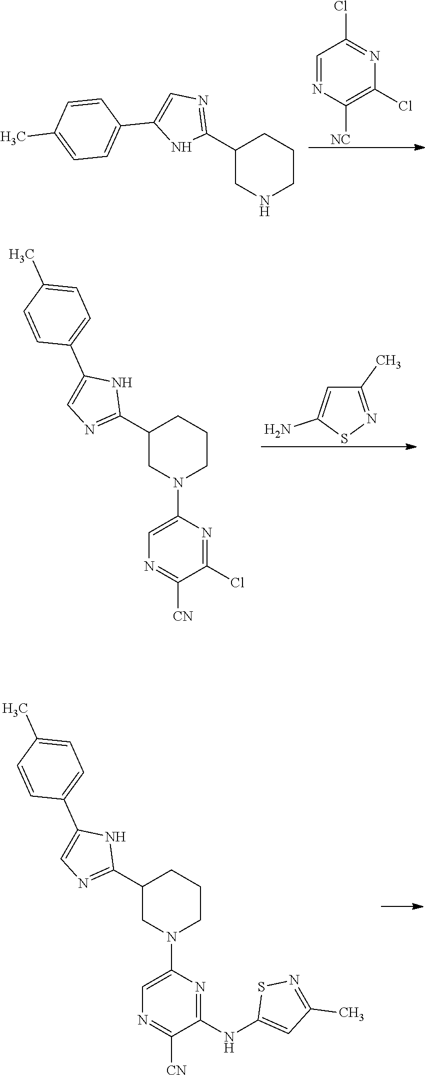

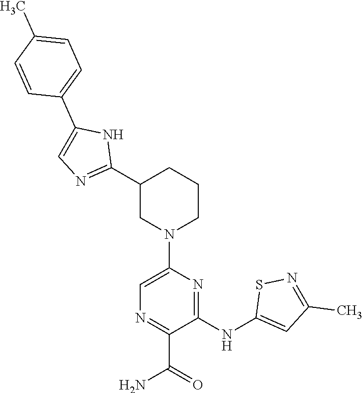 [SMILES: Cc1ccc(-c2cnc(C3CCCN(c4cnc(C(N)=O)c(Nc5cc(C)ns5)n4)C3)[nH]2)cc1]

In a similar manner as described in Example D-131, 3-[(3-methyl-1,2-thiazol-5-yl)amino]-5-{3-[5-(4-methylphenyl)-1H-imidazol-2-yl]piperidin-1-yl}pyrazine-2carboxamide was prepared using 1-[(tert-butoxy)carbonyl]piperidine-3-carboxylic acid. 3-[(3-methyl-1,2-thiazol-5-yl)amino]-5-{3-[5-(4-methylphenyl)-1H-imidazol-2-yl]piperidin-1-yl}pyrazine-2-carboxamide, was submitted to chiral separation to give 3-[(3-methyl-1,2-thiazol-5-yl)amino]-5-[(3S)-3-[5-(4-methylphenyl)-1H-imidazol-2-yl]piperidin-1-yl]pyrazine-2-carboxamide (D-136) (10.0 mg, 10% yield) as yellow solid. MS found for C24H26N8OS as (M+H)⁺ 475.13.

¹H NMR (500 MHz, DMSO) δ 12.29 (s, 1H), 12.14-11.89 (m, 1H), 7.97-7.86 (m, 2H), 7.55 (br. s., 1H), 7.70-7.49 (m, 2H), 7.49-7.17 (m, 1H), 7.23-7.09 (m, 2H), 6.84 (s, 1H), 4.88-4.45 (m, 2H), 3.57-3.20 (m, 2H), 3.04-2.92 (m, 1H), 2.36-2.23 (m, 6H), 2.21-2.09 (m, 1H), 2.03-1.84 (m, 2H), 1.75-1.58 (m, 1H).

### Example D-137: Synthesis of 3-[(3-methyl-1,2-thiazol-5-yl)amino]-5-[(3R)-3-[5-(4-methylphenyl)-1H-imidazol-2-yl]piperidin-1-yl]pyrazine-2-carboxamide (D-137)

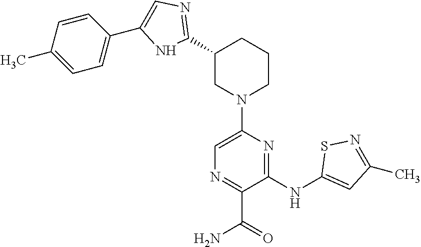

In the same experimental procedure as in Example D-136, 3-[(3-methyl-1,2-thiazol-5-yl)amino]-5-[(3R)-3-[5-(4-methylphenyl)-1H-imidazol-2-yl]piperidin-1-yl]pyrazine-2-carboxamide (D-137) was prepared. MS found for C24H26N8OS as (M+H)⁺ 475.51.

¹H NMR (500 MHz, DMSO) δ 12.29 (s, 1H), 12.14-11.89 (m, 1H), 7.97-7.86 (m, 2H), 7.55 (br. s., 1H), 7.70-7.49 (m, 2H), 7.49-7.17 (m, 1H), 7.23-7.09 (m, 2H), 6.84 (s, 1H), 4.88-4.45 (m, 2H), 3.57-3.20 (m, 2H), 3.04-2.92 (m, 1H), 2.36-2.23 (m, 6H), 2.21-2.09 (m, 1H), 2.03-1.84 (m, 2H), 1.75-1.58 (m, 1H).

### Example D-138: Synthesis of 5-[(2R,3R)-3-(4-cyclopropyl-2-fluorobenzamido)-2-methylpiperidin-1-yl]-3-{[4-(1-cyclopropyl-4-methylpiperidin-4-yl)phenyl]amino}pyrazine-2-carboxamide (D-138)

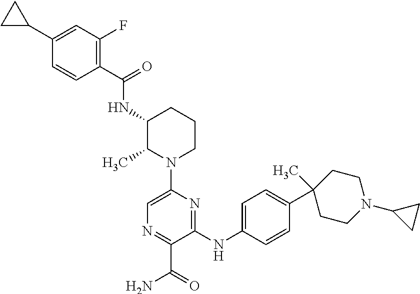 [SMILES: C[C@@H]1[C@H](NC(=O)c2ccc(C3CC3)cc2F)CCCN1c1cnc(C(N)=O)c(Nc2ccc(C3(C)CCN(C4CC4)CC3)cc2)n1]

In a similar manner as described in Example 65, 5-[(2R,3R)-3-(4-cyclopropyl-2-fluorobenzamido)-2-methylpiperidin-1-yl]-3-{[4-(1-cyclopropyl-4-methylpiperidin-4-yl)phenyl]amino}pyrazine-2-carboxamide (D-138) was prepared using N-[(2R,3R)-1-(6-chloro-5-cyanopyrazin-2-yl)-2-methylpiperidin-3-yl]-4-cyclopropyl-2-fluorobenzamide. MS found for C36H44FN7O2 as (M+H)⁺ 626.31.

¹H NMR (500 MHz, DMSO) δ 11.18 (s, 1H), 8.31 (d, J=7.68 Hz, 1H), 7.75 (br. s., 1H), 7.65 (s, 1H), 7.55 (d, J=8.64 Hz, 2H), 7.51-7.44 (m, 1H), 7.33 (br. s., 1H), 7.22 (d, J=8.64 Hz, 2H), 7.05-6.96 (m, 2H), 5.17 (br. s., 1H), 4.26-3.98 (m, 2H), 3.07 (t, J=12.08 Hz, 1H), 2.55-2.33 (m, 4H), 2.08-1.96 (m, 1H), 1.92-1.76 (m, 4H), 1.72-1.54 (m, 2H), 1.54-1.43 (m, 3H). 1.11 (d, J=6.86 Hz, 3H), 1.09-1.02 (m, 5H), 0.82-0.71 (m, 2H), 0.40-0.30 (m, 2H).

### Example D-139: Synthesis of 5-[(2R,3R)-3-(4-cyclopropyl-2-fluorobenzamido)-2-methylpiperidin-1-yl]-3-{[4-(pyrrolidine-1-sulfonyl)phenyl]amino}pyrazine-2-carboxamide (D-139)

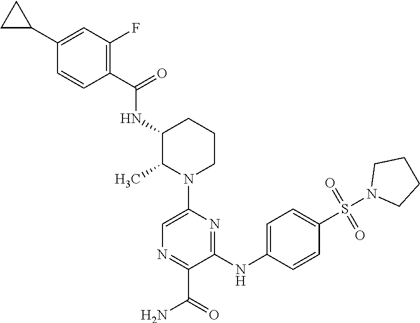 [SMILES: C[C@@H]1[C@H](NC(=O)c2ccc(C3CC3)cc2F)CCCN1c1cnc(C(N)=O)c(Nc2ccc(S(=O)(=O)N3CCCC3)cc2)n1]

In a similar manner as described in Example 65, 5-[(2R,3R)-3-(4-cyclopropyl-2-fluorobenzamido)-2-methylpiperidin-1-yl]-3-{[4-(pyrrolidine-1-sulfonyl)phenyl]amino}pyrazine-2-carboxamide (D-139) was prepared using N-[(2R,3R)-1-(6-chloro-5-cyanopyrazin-2-yl)-2-methylpiperidin-3-yl]-4-cyclopropyl-2-fluorobenzamide. MS found for C31H36FN7O4S as (M+H)⁺ 626.31.

¹H NMR (500 MHz, DMSO) δ 11.75 (s, 1H), 8.34 (d, J=7.13 Hz, 1H), 7.86 (d, J=8.92 Hz, 3H), 7.78 (s, 1H), 7.70 (d, J=8.78 Hz, 2H), 7.55-7.39 (m, 2H), 7.04-6.93 (m, 2H), 5.28 (br. s., 1H), 4.14 (br. s., 1H), 4.03 (td, J=12.14, 4.53 Hz, 1H), 3.17-3.06 (m, 1H), 3.03-2.91 (m, 4H), 2.07-1.96 (m, 1H), 1.93-1.77 (m, 2H), 1.73-1.45 (m. 6H), 1.13 (d, J=6.86 Hz, 3H). 1.07-0.98 (m, 2H), 0.79-0.70 (m, 2H).

### Example D-140: Synthesis of 5-[(2R,3R)-3-(4-cyclopropylbenzamido)-2-methylpiperidin-1-yl]-3-{[3-(piperidin-1-ylmethyl)-1,2-thiazol-5-yl]amino}pyrazine-2-carboxamide (D-140)

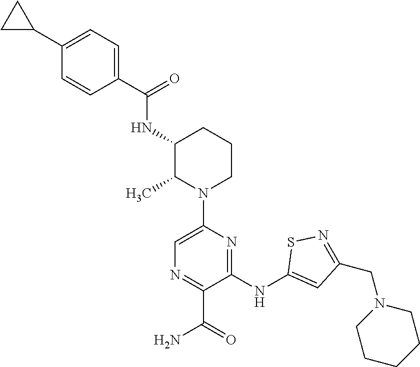 [SMILES: C[C@@H]1[C@H](NC(=O)c2ccc(C3CC3)cc2)CCCN1c1cnc(C(N)=O)c(Nc2cc(CN3CCCCC3)ns2)n1]

In a similar manner as described in Example 65, 5-[(2R,3R)-3-(4-cyclopropylbenzamido)-2-methylpiperidin-1-yl]-3-{[3-(piperidin-1-ylmethyl)-1,2-thiazol-5-yl]amino}pyrazine-2-carboxamide (D-140) was prepared using N-[(2R,3R)-1-(6-chloro-5-cyanopyrazin-2-yl)-2-methylpiperidin-3-yl]-4-cyclopropyl-2-fluorobenzamide. MS found for C30H38N8O2S as (M+H)⁺ 575.18.

¹H NMR (400 MHz, DMSO) δ 12.32 (s, 1H), 8.34 (d, J=7.43 Hz, 1H), 7.91 (br. s., 1H), 7.85-7.73 (m, 3H), 7.56 (br. s., 1H), 7.17 (d, J=8.61 Hz, 2H), 6.91 (s, 1H), 5.34-4.17 (m, 2H), 4.07 (m, J=9.39, 4.70 Hz, 1H), 3.48-3.34 (m, 2H), 3.24-3.11 (m, 1H), 2.33 (br. s., 1H), 2.07-1.83 (m, 3H), 1.76-1.57 (m, 2H), 1.49 (quin, J=5.38 Hz, 4H), 1.37 (d, J=4.70 Hz, 2H), 1.22 (d, J=7.04 Hz, 3H), 1.06-0.98 (m, 2H), 0.78-0.70 (m, 2H).

### Example D-141: Synthesis of 5-[(3R)-3-(6-cyclopropyl-1-oxo-1,2-dihydroisoquinolin-2-yl)piperidin-1-yl]-3-{[1-(1-methylpiperidin-4-yl)-1H-pyrazol-4-yl]amino}pyrazine-2-carboxamide (D-141)

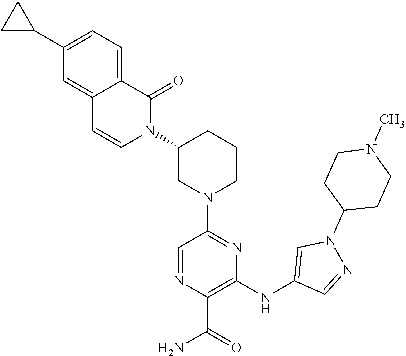 [SMILES: CN1CCC(n2cc(Nc3nc(N4CCC[C@@H](n5ccc6cc(C7CC7)ccc6c5=O)C4)cnc3C(N)=O)cn2)CC1]

In a similar manner as described in Example D-165, 5-[(3R)-3-(6-cyclopropyl-1-oxo-1,2-dihydroisoquinolin-2-yl)piperidin-1-yl]-3-{[1-(1-methylpiperidin-4-yl)-1H-pyrazol-4-yl]amino}pyrazine-2-carboxamide (D-141) was prepared using 1-(1-methylpiperidin-4-yl)-1H-pyrazol-4-amine. MS found for C31H37N9O2 as (M+H)⁺ 568.56.

¹H NMR (400 MHz, DMSO) δ 10.81 (s, 1H), 8.12 (d, J=8.61 Hz, 1H), 7.86 (s, 1H), 7.72 (br. s., 1H), 7.68 (s, 1H), 7.62 (d, J=7.83 Hz, 1H), 7.45 (s, 1H), 7.38 (d, J=1.57 Hz, 1H), 7.31 (br. s., 1H), 7.23 (dd, J=8.41, 1.76 Hz, 1H), 6.64 (d, J=7.43 Hz, 1H), 4.90 (t, J=11.74 Hz, 1H), 4.58 (d, J=1.74 Hz, 1H), 4.37 (d, J=13.30, 1H), 3.58 (br. s., 1H), 3.42-3.35 (m, 3H), 3.20-3.06 (m, 1H), 2.48-2.21 (m, 2H), 2.21-2.12 (m, 1H), 2.10-2.03 (m, 1H), 1.98 (s, 3H), 1.92 (d, J=10.56 Hz, 2H), 1.78-1.63 (m, 3H), 1.60-1.18 (m, 4H), 1.11-1.03 (m, 2H), 0.87-0.78 (m, 2H).

### Example D-142: Synthesis of 5-[(2S,5R)-5-(4-cyclopropylbenzamido)-2-methylpiperidin-1-yl]-3-[(1-methyl-1H-pyrazol-4-yl)amino]pyrazine-2-carboxamide (D-142)

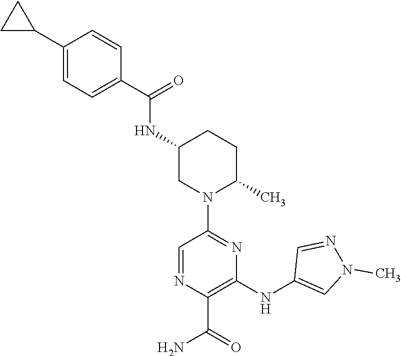 [SMILES: C[C@H]1CC[C@@H](NC(=O)c2ccc(C3CC3)cc2)CN1c1cnc(C(N)=O)c(Nc2cnn(C)c2)n1]

In a similar manner as described in Example D-181, 5-[5-(4-cyclopropylbenzamido)-2-methylpiperidin-1-yl]-3-[(1-methyl-1H-pyrazol-4-yl)amino]pyrazine-2-carboxamide was prepared using 4-cyclopropylbenzoic acid. 5-[5-(4-cyclopropylbenzamido)-2-methylpiperidin-1-yl]-3-[(1-methyl-1H-pyrazol-4-yl)amino]pyrazine-2-carboxamide, was submitted to chiral separation to give 5-[(2S,5R)-5-(4-cyclopropylbenzamido)-2-methylpiperidin-1-yl]-3-[(1-methyl-1H-pyrazol-4-yl)amino]pyrazine-2-carboxamide (D-142) (102.0 mg. 13% yield) as yellow solid. MS found for C25H30N8O2 as (M+H)⁺ 475.48.

¹H NMR (400 MHz, CDCl₃) δ 10.85 (s, 1H), 8.34 (d, J=7.56 Hz, 1H), 7.96 (s, 1H), 7.81 (d, J=8.22 Hz, 2H), 7.68 (br. s., 1H), 7.58 (s, 1H), 7.48 (s, 1H), 7.27 (br. s., 1H), 7.18 (d, J=8.33 Hz, 2H), 4.77-4.51 (m, 2H), 3.97-3.84 (m, 1H), 3.74 (s, 3H), 2.85 (t, J=12.00 Hz, 1H), 2.05-1.64 (m, 5H), 1.25 (d, J=6.69 Hz, 3H), 1.08-0.95 (m, 2H), 0.82-0.68 (m, 2H).

### Example D-143: Synthesis of 5-[(2R,5R)-5-(4-cyclopropylbenzamido)-2-methylpiperidin-1-yl]-3-[(1-methyl-1H-pyrazol-4-yl)amino]pyrazine-2-carboxamide (D-143)

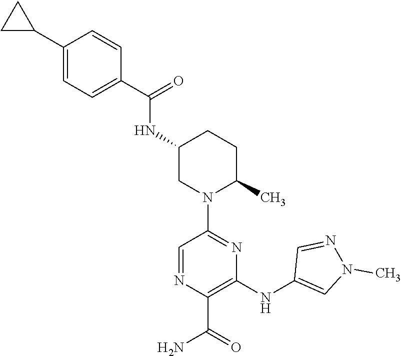 [SMILES: C[C@@H]1CC[C@@H](NC(=O)c2ccc(C3CC3)cc2)CN1c1cnc(C(N)=O)c(Nc2cnn(C)c2)n1]

In the same experimental procedure as in Example D-142, 5-[(2R,5R)-5-(4-cyclopropylbenzamido)-2-methylpiperidin-1-yl-3-(1-methyl-1H-pyrazol-4-yl)amino]pyrazine-2-carboxamide (D-143) was prepared. MS found for C24H26N8OS as (M+H)⁺ 475.47.

¹H NMR (400 MHz, CDCl₃) δ 10.81 (s, 1H), 8.14 (d, J=6.47 Hz, 1H), 7.85 (s, 1H), 7.60 (d, J=8.33 Hz, 3H), 7.51-7.40 (m, 2H), 7.18 (d, J=1.75 Hz, 1H), 7.06 (d, J=8.33 Hz, 2H), 4.68-4.50 (m, 2H), 4.23 (br. s., 1H), 3.81 (s, 3H), 3.37-3.21 (m, 1H), 2.32-2.14 (m, 1H), 2.12-1.97 (m, 1H), 1.97-1.85 (m, 1H), 1.77-1.62 (m, 1H), 1.56-1.41 (m, 1H), 1.25 (d, J=6.58 Hz, 3H), 0.99-0.91 (m, 2H), 0.71-0.63 (m, 2H).

### Example D-144: Synthesis of 5-[(2S,5S)-5-(4-cyclopropylbenzamido)-2-methylpiperidin-1-yl]-3-[(1-methyl-1H-pyrazol-4-yl)amino]pyrazine-2-carboxamide (D-144)

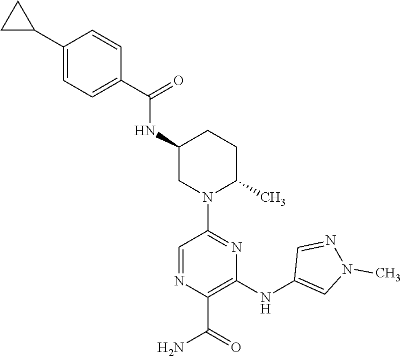 [SMILES: C[C@H]1CC[C@H](NC(=O)c2ccc(C3CC3)cc2)CN1c1cnc(C(N)=O)c(Nc2cnn(C)c2)n1]

In the same experimental procedure as in Example D-142, 5-[(2S,5S)-5-(4-cyclopropylbenzamido)-2-methylpiperidin-1-yl]-3-[(1-methyl-1H-pyrazol-4-yl)amino]pyrazine-2-carboxamide (D-144) was prepared. MS found for C24H26N8OS as (M+H)⁺ 475.47.

¹H NMR (400 MHz, CDCl₃) δ 10.81 (s, 1H), 8.14 (d, J=6.47 Hz, 1H), 7.85 (s, 1H), 7.60 (d, J=8.33 Hz, 3H), 7.51-7.40 (m, 2H), 7.18 (d, J=1.75 Hz, 1H), 7.06 (d, J=8.33 Hz, 2H), 4.68-4.50 (m, 2H), 4.23 (br. s., 1H), 3.81 (s, 3H), 3.37-3.21 (m, 1H), 2.32-2.14 (m, 1H), 2.12-1.97 (m, 1H), 1.97-1.85 (m, 1H), 1.77-1.62 (m, 1H), 1.56-1.41 (m, 1H), 1.25 (d, J=6.58 Hz, 3H), 0.99-0.91 (m, 2H), 0.71-0.63 (m, 2H).

### Example D-145: Synthesis of 5-[(2R,5S)-5-(4-cyclopropylbenzamido)-2-methylpiperidin-1-yl]-3-[(1-methyl-1H-pyrazol-4-yl)amino]pyrazine-2-carboxamide (D-145)

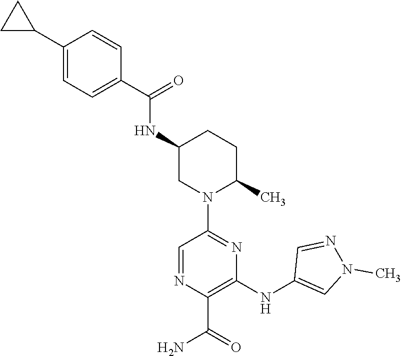 [SMILES: C[C@@H]1CC[C@H](NC(=O)c2ccc(C3CC3)cc2)CN1c1cnc(C(N)=O)c(Nc2cnn(C)c2)n1]

In the same experimental procedure as in Example D-142, 5-[(2R,5S)-5-(4-cyclopropylbenzamido)-2-methylpiperidin-1-yl]-3-[(1-methyl-1H-pyrazol-4-yl)amino]pyrazine-2-carboxamide (D-145) was prepared. MS found for C24H26N8OS as (M+H)⁺ 475.55.

¹H NMR (400 MHz, CDCl₃) δ 10.85 (s, 1H), 8.34 (d, J=7.56 Hz, 1H), 7.96 (s, 1H), 7.81 (d, J=8.22 Hz, 2H), 7.68 (br. s., 1H), 7.58 (s, 1H), 7.48 (s, 1H), 7.27 (br. s., 1H), 7.18 (d, J=8.33 Hz, 2H), 4.77-4.51 (m, 2H), 3.97-3.84 (m, 1H), 3.74 (s, 3H), 2.85 (t, J=12.00 Hz, 1H), 2.05-1.64 (m, 5H), 1.25 (d, J=6.69 Hz, 3H), 1.08-0.95 (m, 2H), 0.82-0.68 (m, 2H).

### Example D-146: Synthesis of 5-[(2R,3R)-3-(4-cyclopropylbenzamido)-2-methylpiperidin-1-yl]-3-{[3-fluoro-4-(4-methylpiperazin-1-yl)phenyl]amino}pyrazine-2-carboxamide (D-146)

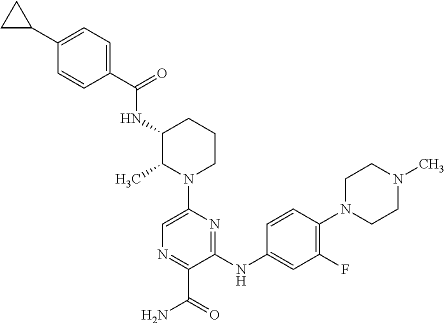 [SMILES: C[C@@H]1[C@H](NC(=O)c2ccc(C3CC3)cc2)CCCN1c1cnc(C(N)=O)c(Nc2ccc(N3CCN(C)CC3)c(F)c2)n1]

In a similar manner as described in Example 8, 5-[(2R,3R)-3-(4-cyclopropylbenzamido)-2-methylpiperidin-1-yl]-3-{[3-fluoro-4-(4-methylpiperazin-1-yl)phenyl]amino}pyrazine-2-carboxamide (D-146) was prepared using 5-[(2R,3R)-3-amino-2-methylpiperidin-1-yl]-3-{[3-fluoro-4-(4-methylpiperazin-1-yl)phenyl]amino}pyrazine-2-carboxamide. MS found for C32H39FN8O2 as (M+H)⁺ 587.63.

¹H NMR (400 MHz, DMSO) δ 11.17 (s, 1H), 8.33 (d, J=7.45 Hz, 1H), 7.83-7.74 (m, 3H), 7.66 (s, 1H), 7.47 (d, J=15.13 Hz, 1H), 7.35 (br. s., 1H), 7.28 (d, J=8.33 Hz, 1H), 7.16 (d, J=8.33 Hz, 2H), 6.89 (t, J=9.32 Hz, 1H), 5.14-4.92 (m, 1H), 4.24-3.99 (m, 2H), 3.14-3.01 (m, 1H), 2.83 (br. s., 4H), 2.39 (br. s., 4H), 2.21 (s, 3H), 2.07-1.79 (m, 3H), 1.72-1.48 (m, 2H), 1.12 (d, J=6.80 Hz, 3H), 1.06-0.98 (m, 2H), 0.79-0.71 (m, 2H).

### Example D-147: Synthesis of 5-[3-(4-cyclopropylphenyl)-2-oxo-1-oxa-3,7-diazaspiro[4.5]decan-7-yl]-3-[(1-methyl-1H-pyrazol-4-yl)amino]pyrazine-2-carboxamide (D-147)

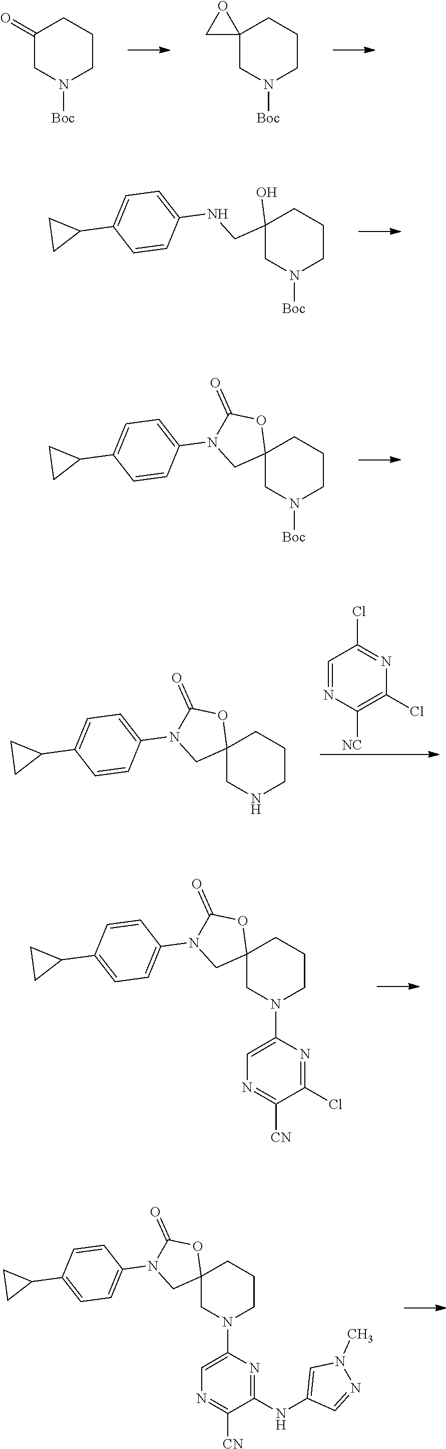

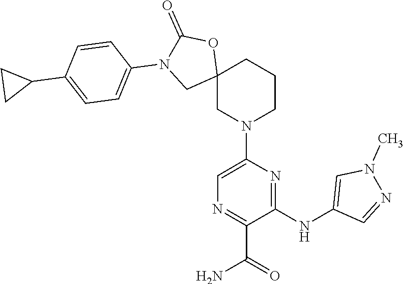 [SMILES: Cn1cc(Nc2nc(N3CCCC4(C3)CN(c3ccc(C5CC5)cc3)C(=O)O4)cnc2C(N)=O)cn1]

Trimethylsulfoxonium iodide (1.1 g, 5.0 mmol) was dissolved in DMSO (5 mL) and stirred at room temperature for 1 h. NaH (0.24 g, 6 mmol) was added and the solution was stirred at 0° C. under nitrogen atmosphere for 1.3 h. To this mixture tert-butyl 3-oxopiperidine-1-carboxylate (1.1 g, 5 mmol) was added and then left at room temperature overnight. The mixture was poured into ice and extracted with ether. The organic phase was separated, washed with water and then brine, dried over Na₂SO₄ and concentrated under reduced pressure. The residue purified by silica flash chromatography with 5 to 50% ethyl acetate in cyclohexane to afford tert-butyl 1-oxa-5-azaspiro[2.5]octane-5-carboxylate (441 mg, 41% yield) as a white solid. MS found for C11H19NO3 as (M+H)⁺ 214.26.

Tert-butyl 1-oxa-5-azaspiro[2.5]octane-5-carboxylate (441 mg, 2.07 mmol) was dissolved in EtOH (5 mL) and 4-cyclopropylaniline (316 mg, 2.37 mmol) were added and the mixture stirred at room temperature overnight. The mixture was concentrated under reduced pressure, dissolved in water (50 mL), and extracted with ethyl acetate (150 mL×3). The organic phase was dried over Na₂SO₄ and concentrated. The residue purified by silica flash chromatography with 0 to 40% ethyl acetate in cyclohexane to give tert-butyl 3-{[(4-cyclopropylphenyl)amino]methyl}-3-hydroxypiperidine-1-carboxylate (239.1 mg, 33% yield). MS found for C₂₀H₃₀N₂O₃ as (M+H)⁺ 347.40.

tert-butyl 3-{[(4-cyclopropylphenyl)amino]methyl}-3-hydroxypiperidine-1-carboxylate (239.1 mg, 0.69 mmol) was dissolved in DCM (2 mL), cooled at 0° C. and TEA (0.55 mL, 4.14 mmol), triphosgene (82.0 mg. 0.276 mmol) dissolved in DCM (2 mL) were added dropwise. The mixture was stirred at room temperature for 4 h. To the mixture a NaHCO₃ sat. aqueous solution was added and stirred for 15 minutes. The mixture was diluted with DCM and extracted. The organic phase was separated, dried over Na₂SO₄ and concentrated. The residue purified by silica flash chromatography with 0 to 50% ethyl acetate in cyclohexane to give tert-butyl 3-(4-cyclopropylphenyl)-2-oxo-1-oxa-3,7-diazaspiro[4.5]decane-7-carboxylate (252.5 mg, 98% yield). MS found for C21H28N2O4 as (M+H)⁺ 373.07.

Tert-butyl 3-(4-cyclopropylphenyl)-2-oxo-1-oxa-3,7-diazaspiro[4.5]decane-7-carboxylate (252.5 mg, 0.678 mmol) was dissolved in DCM (4 ml), and HCl 4 M in dioxane (3 mL, 12.20 mmol) were added and the mixture was stirred for 2 h. The solvent was evaporated to give a white solid which was passed through an SCX cartridge. Evaporation of the ammonia fractions gave 3-(4-cyclopropylphenyl)-1-oxa-3,7-diazaspiro[4.5]decan-2-one (162.5 mg, 88% yield). MS found for C16H20N2O2 as (M+H)⁺ 273.01.

3,5-Dichloropyrazine-2-carbonitrile (108.0 mg. 0.62 mmol) and 3-(4-cyclopropylphenyl)-1-oxa-3,7-diazaspiro[4.5]decan-2-one (162.5 mg, 0.597 mmol) were dissolved in DMF (1.5 mL), DIPEA (210.0 μL, 1.194 mmol) and the mixture was stirred at room temperature overnight under a nitrogen atmosphere. The mixture was poured into ice and extracted with ethyl acetate. The organic phase was separated, dried over Na₂SO₄ and concentrated. The residue purified by silica flash chromatography with 0 to 100% ethyl acetate in cyclohexane to give 3-chloro-5-[3-(4-cyclopropylphenyl)-2-oxo-1-oxa-3,7-diazaspiro[4.5]decan-7-yl]pyrazine-2-carbonitrile (161.5.2 mg, 66% yield). MS found for C21H20ClN5O2 as (M+H)⁺ 409.97.

3-chloro-5-[3-(4-cyclopropylphenyl)-2-oxo-1-oxa-3,7-diazaspiro[4.5]decan-7-yl]pyrazine-2-carbonitrile (80.0 mg, 0.195 mmol), 3-methyl-1,2-thiazol-5-amine hydrochloride (38.0 mg, 0.391 mmol), and Cs₂CO₃ (250.0 mg, 0.782 mmol) were suspended in dioxane (4 mL). (+/−) BINAP (25 mg, 0.039 mmol) and Pd(OAc)₂ (9.0 mg, 0.0391 mmol) were added under nitrogen and the mixture stirred for 2 h at 120° C. The mixture was concentrated and the residue purified by silica flash chromatography with 0 to 100% ethyl acetate in cyclohexane and then with 0 to 20% MeOH in ethyl acetate to give 5-[3-(4-cyclopropylphenyl)-2-oxo-1-oxa-3,7-diazaspiro[4.5]decan-7-yl]-3-[(1-methyl-1H-pyrazol-4-yl)amino]pyrazine-2-carbonitrile (90.3 mg, 98% yield). MS found for C25H26N8O2 as (M+H)⁺ 471.03.

To a suspension of 5-[3-(4-cyclopropylphenyl)-2-oxo-1-oxa-3,7-diazaspiro[4.5]decan-7-yl]-3-[(1-methyl-1H-pyrazol-4-yl)amino]pyrazine-2-carbonitrile (90.3 mg, 0.192 mmol) in MeOH/DMSO (3 mL/0.3 mL), NaOH (18.6 mg, 0.46 mmol), TEA (0.60 mL, 4.30 mmol) and H₂O₂ (0.9 mL) were added. The mixture was stirred for 1 h at room temperature, then it was partitioned between DCM and H₂O. The combined organic phase were dried over Na₂SO₄ and concentrated. The crude was purified by silica flash chromatography with 50 to 100% ethyl acetate in cyclohexane and then with 0 to 20% MeOH in ethyl acetate to give 5-[3-(4-cyclopropylphenyl)-2-oxo-1-oxa-3,7-diazaspiro[4.5]decan-7-yl]-3-[(1-methyl-1H-pyrazol-4-yl)amino]pyrazine-2-carboxamide (D-147) (32.8 mg, 35% yield) as a yellow solid. MS found for C25H28N8O3 as (M+H)⁺ 489.47.

¹H NMR (400 MHz, DMSO) δ 10.80 (s, 1H), 7.75 (s, 1H), 7.71-7.62 (m, 2H), 7.50 (s, 1H), 7.42 (d, J=8.61 Hz, 2H), 7.28 (br. s., 1H), 7.07 (d, J=8.61 Hz, 2H), 4.17 (d, J=13.30 Hz, 1H), 3.96 (m, J=13.30 Hz, 1H), 3.85 (q, J=9.39 Hz, 2H), 3.78 (d, J=13.30 Hz, 1H), 3.63 (s, 3H), 3.59-3.48 (m, 1H), 2.11-2.00 (m, 2H), 1.94-1.77 (m, 2H), 1.73 (d, J=3.52 Hz, 1H), 0.98-0.88 (m, 2H), 0.66-0.59 (m, 2H).

### Example D-148: Synthesis of 3-[(1-methyl-1H-pyrazol-4-yl)amino]-5-{3-[5-(3-methylphenyl)-1H-imidazol-2-yl]piperidin-1-yl}pyrazine-2-carboxamide (D-148)

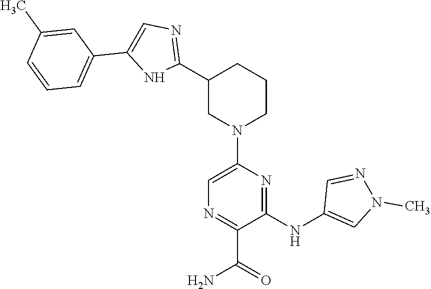

In a similar manner as described in Example D-136, 3-[(1-methyl-1H-pyrazol-4-yl)amino]-5-{3-[5-(3-methylphenyl)-1H-imidazol-2-yl]piperidin-1-yl}pyrazine-2-carboxamide (D-148) was prepared. MS found for C₂₄H₂₇N₉O as (M+H)⁺ 458.51.

¹H NMR (400 MHz, DMSO) δ 12.23-11.85 (m, 1H), 10.83 (s, 1H), 7.86 (s, 1H), 7.73-7.65 (m, 2H), 7.54 (d, J=12.52 Hz, 4H), 7.20 (d, J=15.26 Hz, 2H), 7.07-6.92 (m, 1H), 4.76-4.56 (m, 1H), 4.45-4.26 (m, 1H), 3.76-3.60 (m, 3H), 3.36-3.07 (m, 2H), 3.03-2.90 (m, 1H), 2.36-2.29 (m, 3H), 2.25-1.52 (m, 4H).

### Example D-149: Synthesis of 5-[(2R15 S)-5-(4-cyclopropylbenzamido)-2-methylpiperidin-1-yl]-3-{[3-fluoro-4-(4-methylpiperazin-1-yl)phenyl]amino}pyrazine-2-carboxamide (D-149)

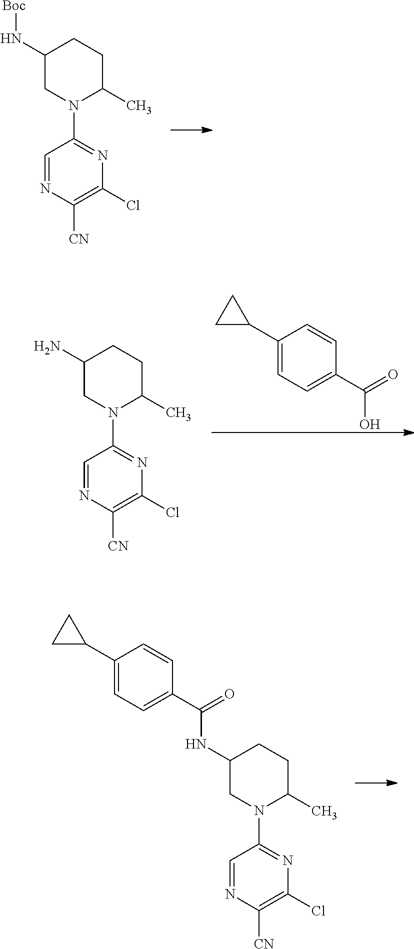 [SMILES: CC1CCC(NC(=O)OC(C)(C)C)CN1c1cnc(C#N)c(Cl)n1 ; CC1CCC(N)CN1c1cnc(C#N)c(Cl)n1 ; O=C(O)c1ccc(C2CC2)cc1 ; CC1CCC(NC(=O)c2ccc(C3CC3)cc2)CN1c1cnc(C#N)c(Cl)n1]

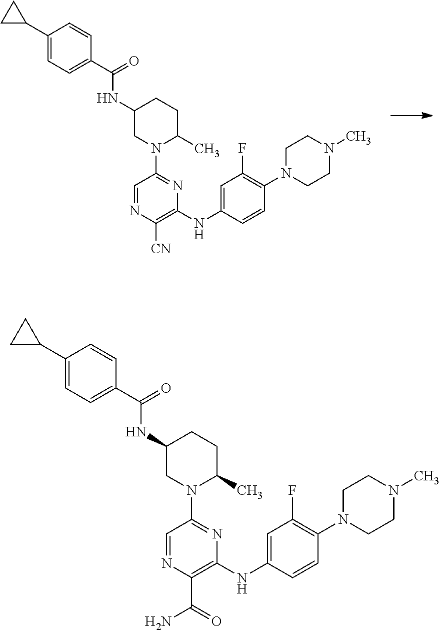 [SMILES: CC1CCC(NC(=O)c2ccc(C3CC3)cc2)CN1c1cnc(C#N)c(Nc2ccc(N3CCN(C)CC3)c(F)c2)n1 ; C[C@@H]1CC[C@H](NC(=O)c2ccc(C3CC3)cc2)CN1c1cnc(C(N)=O)c(Nc2ccc(N3CCN(C)CC3)c(F)c2)n1]

In a similar manner as described in Example D-181, tert-butyl N-[1-(6-chloro-5-cyanopyrazin-2-yl)-6-methylpiperidin-3-yl]carbamate was prepared. MS found for C16H22ClN5O2 as (M+H)⁺ 353.0.

Tert-butyl N-[1-(6-chloro-5-cyanopyrazin-2-yl)-6-methylpiperidin-3-yl]carbamate (1.93 g, 5.48 mmol) was dissolved in HCl 1.25 M (20 mL) and stirred at room temperature for 4 h. The solvent was evaporated to give a white solid which was passed through an SCX cartridge. Evaporation of the ammonia fractions gave (5-(5-amino-2-methylpiperidin-1-yl)-3-chloropyrazine-2-carbonitrile (1.57 g, quant. yield). MS found for C11H14ClN5 as (M+H)⁺ 252.21.

(5-(5-Amino-2-methylpiperidin-1-yl)-3-chloropyrazine-2-carbonitrile (1.57 g, 5.48 mmol) were dissolved in DMF (10 mL), 4-cyclopropylbenzoic acid (1.16 g, 7.12 mmol), DIPEA (5.0 mL, 27.5 mmol) and PyBop (3.72 g, 7.12 mmol) were added. The mixture was stirred for 2 h, and then it was partitioned between ethyl acetate and H₂O. The organic phase was dried with Na₂SO₄, concentrated and purified by silica flash chromatography with 0 to 100% ethyl acetate in cyclohexane to give N-[1-(6-chloro-5-cyanopyrazin-2-yl)-6-methylpiperidin-3-yl]-4-cyclopropylbenzamide (1.76 g, 82% yield). MS found for C21H22ClN5O as (M+H)⁺ 396.40. N-[1-(6-chloro-5-cyanopyrazin-2-yl)-6-methylpiperidin-3-yl]-4-cyclopropylbenzamide (538 mg, 1.36 mmol) were suspended in dioxane (5 mL), 3-fluoro-4-(4-methylpiperazin-1-yl)aniline (428.0 mg, 2.05 mmol), Cs₂CO₃ (1780.32 mg, 5.45 mmol), (+/−) BINAP (170.0 mg, 0.272 mmol) and Pd(OAc)₂ (62.0 mg, 0.272 mmol) were added while degassing with nitrogen. The mixture was heated at 110° C. for 2h. The solvent was evaporated and the residue purified by silica flash chromatography with cyclohexane:ethyl acetate from 80 to 100% to give N-[1-(5-cyano-6-{[3-fluoro-4-(4-methylpiperazin-1-yl)phenyl]amino}pyrazin-2-yl)-6-methylpiperidin-3-yl]-4-cyclopropylbenzamide (452.5 mg, 58% yield). MS found for C32H37FN8O as (M+H)⁺ 569.54. To a suspension of N-[1-(5-cyano-6-{[3-fluoro-4-(4-methylpiperazin-1-yl)phenyl]amino}pyrazin-2-yl)-6-methylpiperidin-3-yl]-4-cyclopropylbenzamide (452.5 mg, 0.795 mmol) in MeOH/DMSO (12 mL 4 mL), NaOH (78.0 mg, 1.91 mmol), TEA (2.2 mL, 15.9 mmol) and H₂O₂ (0.9 mL) were added. The mixture was stirred for 1 h at room temperature, then it partitioned between DCM and H₂O. The combined organic phase were dried over Na₂SO₄ and concentrated. The crude was submitted to chiral separation to give 5-[(2R,5S)-5-(4-cyclopopropylbenzamido)-2-methylpiperidin-1-yl]-3-{[3-fluoro-4-(4-methylpiperazin-1-yl)phenyl]amino}pyrazine-2-carboxamide (D-149) (53.7 mg, 12% yield) as a yellow solid. MS found for C32H39FN8O2 as (M+H)⁺ 587.58.

¹H NMR (400 MHz, DMSO) δ 11.16 (br. s., 1H), 8.34 (d, J=8.07 Hz, 1H), 7.85-7.74 (m, 3H), 7.69 (s, 1H), 7.53-7.40 (m, 1H), 7.36 (br. s., 1H), 7.27 (d, J=7.09 Hz, 1H), 7.17 (d, J=8.31 Hz, 2H), 6.94-6.82 (m, 1H), 4.84-4.32 (m, 2H), 4.01-3.83 (m, 1H), 3.04-2.70 (m, 5H), 2.59-2.34 (m, 4H), 2.26 (br. s., 3H), 2.04-1.95 (m, 1H), 1.95-1.68 (m, 4H), 1.24 (d, J=6.85 Hz, 3H), 1.07-0.98 (m, 2H), 0.80-0.71 (m, 2H).

### Example D-150: Synthesis of 5-[(2S,5R)-5-(4-cyclopropylbenzamido)-2-methylpiperidin-1-yl]-3-{[3-fluoro-4-(4-methylpiperazin-1-yl)phenyl]amino}pyrazine-2-carboxamide (D-150)

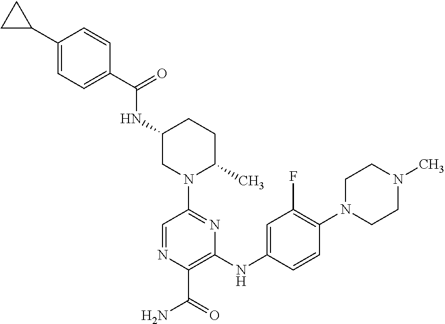 [SMILES: C[C@H]1CC[C@@H](NC(=O)c2ccc(C3CC3)cc2)CN1c1cnc(C(N)=O)c(Nc2ccc(N3CCN(C)CC3)c(F)c2)n1]

In the same experimental procedure as in Example D-149, 5-[(2S,5R)-5-(4-cyclopropylbenzamido)-2-methylpiperidin-1-yl]-3-{[3-fluoro-4-(4-methylpiperazin-1-yl)phenyl]amino}pyrazine-2-carboxamide (D-150) was prepared. MS found for C32H39FN8O2 as (M+H)⁺ 587.57.

¹H NMR (400 MHz, DMSO) δ 11.16 (br. s., 1H), 8.34 (d, J=8.07 Hz, 1H), 7.85-7.74 (m, 3H), 7.69 (s, 1H), 7.53-7.40 (m, 1H), 7.36 (br. s., 1H), 7.27 (d, J=7.09 Hz, 1H), 7.17 (d, J=8.31 Hz, 2H), 6.94-6.82 (m, 1H), 4.84-4.32 (m, 2H), 4.01-3.83 (m, 1H), 3.04-2.70 (m, 5H), 2.59-2.34 (m, 4H), 2.26 (br. s., 3H), 2.04-1.95 (m, 1H), 1.95-1.68 (m, 4H), 1.24 (d, J=6.85 Hz, 3H), 1.07-0.98 (m, 2H), 0.80-0.71 (m, 2H).

### Example D-151: Synthesis of 5-[(2R,5S)-5-[(dimethylcarbamoyl)amino]-2-methylpiperidin-1-yl]-3-[(1-methyl-1H-pyrazol-4-yl)amino]pyrazine-2carboxamide (D-151)

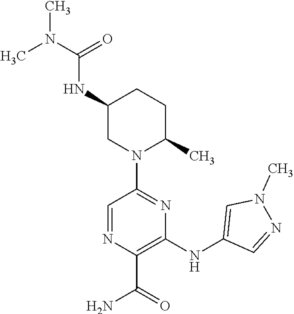 [SMILES: C[C@@H]1CC[C@H](NC(=O)N(C)C)CN1c1cnc(C(N)=O)c(Nc2cnn(C)c2)n1]

In a similar manner as described in Example D-181, 5-[(2R,5S)-5-[(dimethylcarbamoyl)amino]-2-methylpiperidin-1-yl]-3-[(1-methyl-1H-pyrazol-4-yl)amino]pyrazine-2-carboxamide (D-151) was prepared using dimethylcarbamyl chloride. MS found for C18H27N9O2 as (M+H)⁺ 402.08.

¹H NMR (400 MHz, DMSO) δ 10.75 (s, 1H), 7.95 (s, 1H), 7.68-6.77 (m, 4H), 6.00 (d, J=7.04 Hz, 2H), 4.76-4.45 (m, 2H), 3.84 (s, 3H), 3.65-3.45 (m, 1H), 2.85 (s, 6H), 2.81-2.74 (m, 1H), 1.89-1.67 (m, 4H), 1.24 (d, J=6.65 Hz, 3H).

### Example D-152: Synthesis of 5-[(2S,5R)-5-[(dimethylcarbamoyl)amino]-2-methylpiperidin-1-yl]-3-[(1-methyl-1H-pyrazol-4-yl)amino]pyrazine-2-carboxamide (D-150)

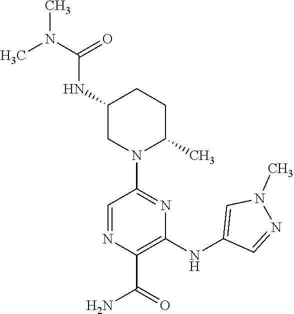 [SMILES: C[C@H]1CC[C@@H](NC(=O)N(C)C)CN1c1cnc(C(N)=O)c(Nc2cnn(C)c2)n1]

In a similar manner as described in Example D-181, 5-[(2S,5R)-5-[(dimethylcarbamoyl)amino]-2-methylpiperidin-1-yl]-3-[(1-methyl-1H-pyrazol-4-yl)amino]pyrazine-2-carboxamide (D-152) was prepared using dimethylcarbamyl chloride. MS found for C18H27N9O2 as (M+H)⁺ 419.

¹H NMR (400 MHz, DMSO) δ 10.84 (s, 1H), 7.97 (s, 1H), 7.67 (br. s., 1H), 7.54 (s, 1H), 7.47 (s, 1H), 7.26 (br. s., 1H), 6.17 (d, J=7.41 Hz, 1H), 4.78-4.40 (m, 2H), 3.83 (s, 3H), 3.60-3.46 (m, 1H), 2.83 (s, 6H), 2.73-2.65 (m, 1H), 1.89-1.64 (m, 4H), 1.22 (d, J=6.86 Hz, 3H).

### Example D-153: Synthesis 5-[(2R,5S)-5-(4-cyclopropylbenzamido)-2-methylpiperidin-1-yl]-3-{[4-(4-methylpiperazin-1-yl)phenyl]amino}pyrazine-2-carboxamide (D-153)

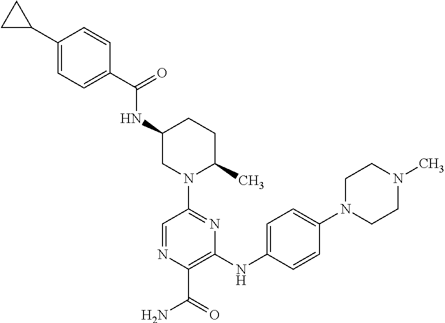 [SMILES: C[C@@H]1CC[C@H](NC(=O)c2ccc(C3CC3)cc2)CN1c1cnc(C(N)=O)c(Nc2ccc(N3CCN(C)CC3)cc2)n1]

In a similar manner as described in Example D-149, 5-[(2R,5S)-5-(4-cyclopropylbenzamido)-2-methylpiperidin-1-yl]-3-{[4-(4-methylpiperazin-1-yl)phenyl]amino}pyrazine-2-carboxamide (D-153) was prepared using 4-(4-methylpiperazin-1-yl)aniline. MS found for C32H40N8O2 as (M+H)⁺ 569.57.

¹H NMR (500 MHz, DMSO) δ 10.89 (br. s., 1H), 8.34 (d, J=7.96 Hz, 1H), 7.84 (d, J=8.23 Hz, 2H), 7.70 (br. s., 1H), 7.62 (s, 1H), 7.40 (d, J=8.78 Hz, 2H), 7.30-7.24 (m, 1H), 7.19 (d, J=8.51 Hz, 2H), 6.80-6.68 (m, 2H), 4.60 (br. s., 2H), 4.01-3.88 (m, 1H), 2.96-2.70 (m. 5H), 2.42-2.26 (m, 4H), 2.20 (s, 3H), 2.05-1.96 (m, 1H), 1.94-1.62 (m, 4H), 1.24 (d, J=6.86 Hz, 3H), 1.08-0.98 (m, 2H), 0.81-0.71 (m, 2H).

### Example D-154: Synthesis 5-[(2S,5R)-5-(4-cyclopropylbenzamido)-2-methylpiperidin-1-yl]-3-{[4-(4-methylpiperazin-1-yl)phenyl]amino}pyrazine-2-carboxamide (D-154)

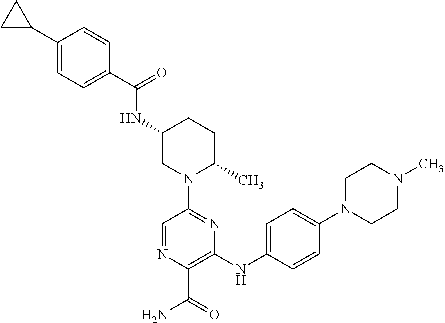 [SMILES: C[C@H]1CC[C@@H](NC(=O)c2ccc(C3CC3)cc2)CN1c1cnc(C(N)=O)c(Nc2ccc(N3CCN(C)CC3)cc2)n1]

In a similar manner as described in Example D-149, 5-[(2S,5R)-5-(4-cyclopropylbenzamido)-2-methylpiperidin-1-yl]-3-{[4-(4-methylpiperazin-1-yl)phenyl]amino}pyrazine-2-carboxamide (D-154) was prepared using 4-(4-methylpiperazin-1-yl)aniline. MS found for C32H40N8O2 as (M+H)⁺ 569.57.

¹H NMR (500 MHz, DMSO) δ 10.89 (br. s., 1H), 8.34 (d, J=7.96 Hz, 1H), 7.84 (d, J=8.23 Hz, 2H), 7.70 (br. s., 1H), 7.62 (s, 1H), 7.40 (d, J=8.78 Hz, 2H), 7.30-7.24 (m, 1H), 7.19 (d, J=8.51 Hz, 2H), 6.80-6.68 (m, 2H), 4.60 (br. s., 2H), 4.01-3.88 (m, 1H), 2.96-2.70 (m, 5H), 2.42-2.26 (m, 4H), 2.20 (s, 3H), 2.05-1.96 (m, 1H), 1.94-1.62 (m, 4H), 1.24 (d, J=6.86 Hz, 3H), 1.08-0.98 (m, 2H), 0.81-0.71 (m, 2H).

### Example D-155: 5-[(3R)-3-[6-(dimethylamino)-1-oxo-1,2-dihydroisoquinolin-2-yl]piperidin-1-yl]-3-[(1-methyl-1H-pyrazol-4-yl)amino]pyrazine-2-carboxamide (D-155)

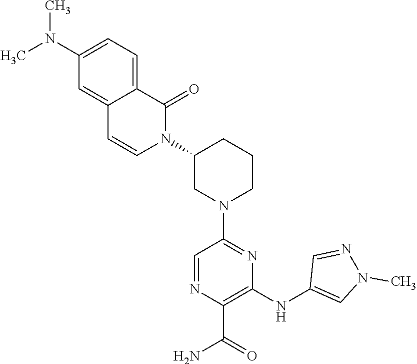

In a similar manner as described in Example D-165, 5-[(3R)-3-[6-(dimethylamino)-1-oxo-1,2-dihydroisoquinolin-2-yl]piperidin-1-yl]-3-[(1-methyl-1H-pyrazol-4-yl)amino]pyrazine-2-carboxamide (D-155) was prepared using 1-methyl-1H-pyrazol-4-amine. MS found for C25H29N9O2 as (M+H)⁺ 488.38.

¹H NMR (400 MHz, DMSO) δ 10.82 (s, 1H), 8.05 (d, J=9.10 Hz, 1H), 7.88 (s, 1H), 7.71 (br. s., 1H), 7.66 (s, 1H), 7.52-7.43 (m, 2H), 7.30 (br. s., 1H), 6.96 (dd, J=9.10, 2.52 Hz, 1H), 6.70 (d, J=2.52 Hz, 1H), 6.52 (d, J=7.56 Hz, 1H), 4.96-4.80 (m, 1H), 4.55 (d, J=10.96 Hz, 1H). 4.39 (d, J=12.50 Hz, 1H), 3.59 (s, 3H), 3.29-3.17 (m, 1H), 3.17-2.98 (m, 7H), 2.24-2.07 (m, 1H), 1.92 (br. s., 2H), 1.76-1.57 (m, 1H).

### Example D-156: 5-[(3R)-3-[6-(dimethylamino)-1-oxo-1,2,3,4-tetrahydroisoquinolin-2-yl]piperidin-1-yl]-3-[(1-methyl-1H-pyrazol-4-yl)amino]pyrazine-2-carboxamide (D-156)

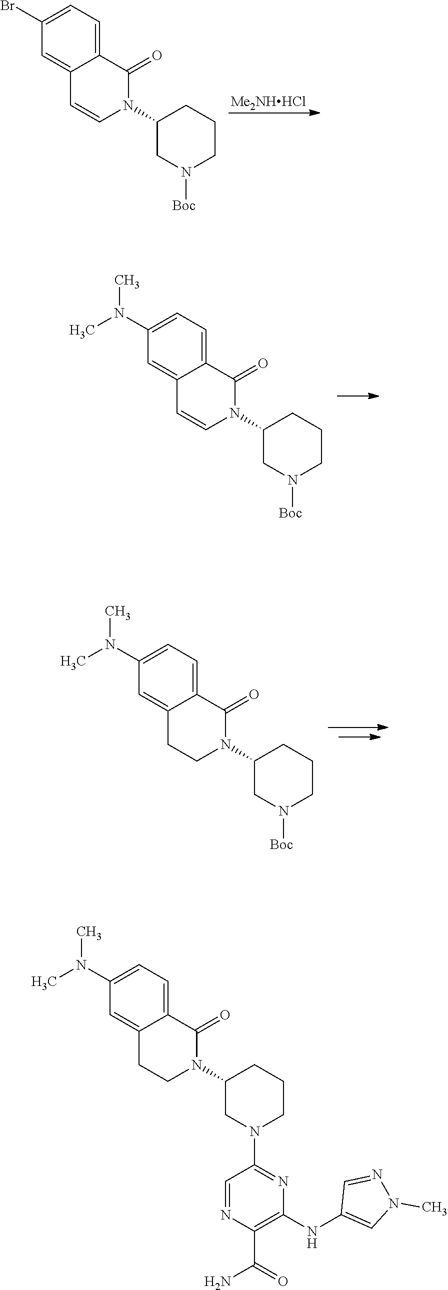

To a solution of tert-butyl (3R)-3-(6-bromo-1-oxoisoquinolin-2-yl)piperidine-1-carboxylate (498.4 mg, 1.23 mmol) in t-BuOH (5 ml), N-methylmethamine hydrochloride (445.0, 5.46 mmol) was added and the solution was degassed. Then Pd(OAc)₂ (20.0 mg, 0.089 mmol), JohnPhos (66.0 mg, 0.22 mmol), sodium t-butoxide (820.0 mg, 8.53 mmol) were added and the mixture was stirred at 90° C. overnight, then left to reach room temperature, absorbed on silica gel column and the crude was purified by silica flash chromatography with 50 to 100% ethyl acetate in cyclohexane to give tert-butyl (3R)-3-[6-(dimethylamino)-1-oxo-1,2-dihydroisoquinolin-2-yl]piperidine-1-carboxylate (168.0 mg, 36% yield). MS found for C21H29N3O3 as (M+H)⁺ 372.06.

To a solution of tert-butyl (3R)-3-[6-(dimethylamino)-1-oxo-1,2-dihydroisoquinolin-2-yl]piperidine-1-carboxylate (168.0 mg, 0.45 mmol) in EtOH (40 ml), Pd/C (130 mg) were added and the mixture was stirred under hydrogen atmosphere (6 psi) at 70° C. for 2 days. The mixture was left to reach room temperature then filtered and evaporated to dryness to give tert-butyl (3R)-3-[6-(dimethylamino)-1-oxo-1,2,3,4-tetrahydroisoquinolin-2-yl]piperidine-1-carboxylate (160.8 mg, 95% yield). MS found for C21H31N3O3 as (M+H)⁺ 374.08.

In a similar manner as described in Example D-165, 5-[(3R)-3-[6-(dimethylamino)-1-oxo-1,2,3,4-tetrahydroisoquinolin-2-yl]piperidin-1-yl]-3-[(1-methyl-1H-pyrazol-4-yl)amino]pyrazine-2-carboxamide (D-156) was prepared using 1-methyl-1H-pyrazol-4-amine. MS found for C25H31N9O2 as (M+H)⁺ 490.43.

¹H NMR (400 MHz, DMSO) δ 10.80 (s, 1H), 7.89 (s, 1H), 7.75-7.65 (m, 2H), 7.62 (s, 1H), 7.46 (s, 1H), 7.28 (br. s., 1H), 6.64 (dd, J=8.88, 2.52 Hz, 1H), 6.52 (d, J=2.19 Hz, 1H), 4.61-4.48 (m, 1H), 4.44 (d, J=11.29 Hz, 1H), 4.34 (d, J=13.04 Hz, 1H), 3.66-3.44 (m, 5H), 3.13 (t, J=11.84 Hz, 1H), 2.04-1.72 (m, 3H), 1.69-1.51 (m, 1H).

### Example D-157: Synthesis of 3-[(2S,5R)-5-[4-(2-hydroxypropan-2-yl)benzamido]-2-methylpiperidin-1-yl]-5-[(1-methyl-1H-pyrazol-4-yl)amino]-1,2,4-triazine-6-carboxamide (D-157)

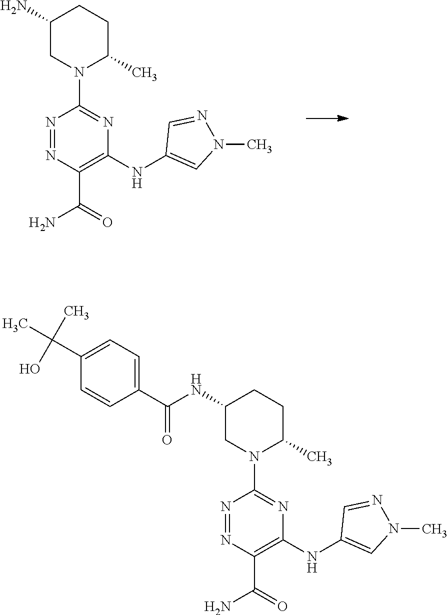 [SMILES: C[C@H]1CC[C@@H](N)CN1c1nnc(C(N)=O)c(Nc2cnn(C)c2)n1 ; C[C@H]1CC[C@@H](NC(=O)c2ccc(C(C)(C)O)cc2)CN1c1nnc(C(N)=O)c(Nc2cnn(C)c2)n1]

In a similar manner as described in Example D-277, 3-[(2S,5R)-5-amino-2-methylpiperidin-1-yl]-5-[(1-methyl-1H-pyrazol-4-yl)amino]-1,2,4-triazine-6-carboxamide was prepared. MS found for C14H21N9O as (M+H)⁺ 332.10.

3-[(2S,5R)-5-amino-2-methylpiperidin-1-yl]-5-[(1-methyl-1H-pyrazol-4-yl)amino]-1,2,4-triazine-6-carboxamide (35.0 mg, 0.106 mmol) was dissolved in DMF (3 ml), 4-(2-hydroxypropan-2-yl)benzoic acid (29.9 mg, 0.166 mmol). DIPEA (0.06 mL, 0.318 mmol) and TBTU (43.0 mg, 0.132 mmol) were added. The mixture was stirred at room temperature overnight. Then was poured into water and extracted with ethyl acetate. The organic phase was separated, dried over Na₂SO₄ and concentrated. The residue was purified by silica flash chromatography with 0 to 100% ethyl acetate in cyclohexane and then with 0 to 20% MeOH in ethyl acetate to give 3-[(2S,5R)-5-[4-(2-hydroxypropan-2-yl)benzamido]-2-methylpiperidin-1-yl]-5-[(1-methyl-1H-pyrazol-4-yl)amino]-1,2,4-triazine-6-carboxamide (D-157, 26.7 mg, 51% yield) as a yellow solid. MS found for C24H31N9O3 as (M+H)⁺ 494.39.

¹H NMR (400 MHz, DMSO) δ 10.99 (s, 1H), 8.57-7.93 (m, 3H), 7.86 (d, J=7.02 Hz, 2H), 7.75-7.51 (m, 4H), 5.45-4.95 (m, 2H), 4.83 (d, J=8.11 Hz, 1H), 3.98-3.78 (m, 4H), 3.01-2.78 (m, 1H), 2.05-1.70 (m, 4H), 1.44 (s, 6H), 1.27 (d, J=7.02 Hz, 3H).

### Example D-158: Synthesis of 5-[(3R)-3-[4-(2-hydroxypropan-2-yl)benzamido]piperidin-1-yl]-3-[(3-methyl-1,2-thiazol-5-yl)amino]pyrazine-2-carboxamide (D-158)

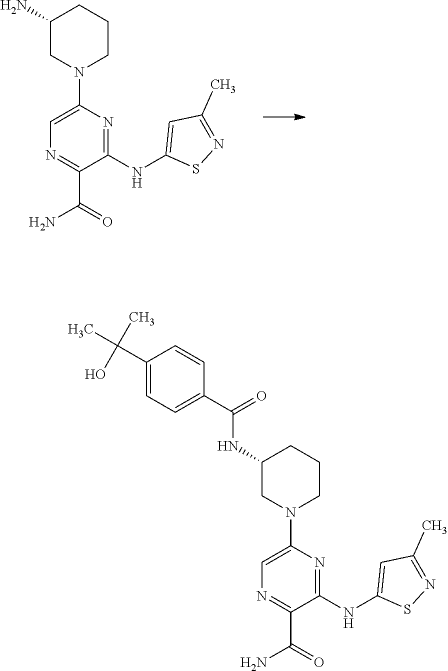 [SMILES: Cc1cc(Nc2nc(N3CCC[C@@H](N)C3)cnc2C(N)=O)sn1 ; Cc1cc(Nc2nc(N3CCC[C@@H](NC(=O)c4ccc(C(C)(C)O)cc4)C3)cnc2C(N)=O)sn1]

In a similar manner as described in Example D-216, 5-[(3R)-3-aminopiperidin-1-yl]-3-[(3-methyl-1,2-thiazol-5-yl)amino]pyrazine-2-carboxamide was prepared. MS found for C14H19N7OS as (M+H)⁺ 334.18.

5-[(3R)-3-aminopiperidin-1-yl]-3-[(3-methyl-1,2-thiazol-5-yl)amino]pyrazine-2-carboxamide (40.0 mg, 0.12 mmol) was dissolved in DMF (3 ml), 4-(2-hydroxypropan-2-yl)benzoic acid (26.4 mg, 0.146 mmol), DIPEA (0.07 mL, 0.40 mmol) and TBTU (53.3 mg, 0.166 mmol) were added. The mixture was stirred at room temperature overnight. Then was poured into water and extracted with ethyl acetate. The organic phase was separated, dried over Na₂SO₄ and concentrated. The residue purified by silica flash chromatography with 20 to 100% ethyl acetate in cyclohexane and then purified again with 0 to 20%0 MeOH in ethyl acetate. The compound was purified again by silica C18 flash chromatography with 5 to 40% CH₃CN in H₂O with 0.1% HCOOH to give 5-[(3R)-3-[4-(2-hydroxypropan-2-yl)benzamido]piperidin-1-yl]-3-[(3-methyl-1,2-thiazol-5-yl)amino]pyrazine-2-carboxamide (D-158) (13.4 mg, 22% yield) as a yellow solid. MS found for C24H29N7O3S as (M+H)⁺ 496.29.

¹H NMR (500 MHz, DMSO) δ 12.29 (s, 1H), 8.37 (d, J=7.41 Hz, 1H), 7.91 (br. s., 1H) 7.84 (s, 1H), 7.76 (d, J=8.51 Hz, 2H), 7.53 (d, J=8.51 Hz, 3H), 6.85 (s, 1H), 5.11 (s, 1H), 4.61-4.28 (m, 2H), 4.08-3.82 (m, 1H), 3.31-3.22 (m, 2H), 2.28 (s, 3H), 2.08-1.87 (m, 2H), 1.82-1.57 (m, 2H), 1.43 (s, 6H).

### Example D-159: Synthesis of 5-[(2R,3R)-3-[4-(2-hydroxypropan-2-yl)benzamido]-2-methylpiperidin-1-yl]-3-[(3-methyl-1,2-thiazol-5-yl)amino]pyrazine-2-carboxamide (D-159)

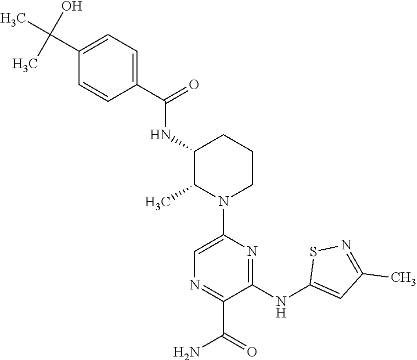 [SMILES: Cc1cc(Nc2nc(N3CCC[C@@H](NC(=O)c4ccc(C(C)(C)O)cc4)[C@H]3C)cnc2C(N)=O)sn1]

In a similar manner as described in Example D-216 5-[(2R,3R)-3-[4-(2-hydroxypropan-2-yl)benzamido]-2-methylpiperidin-1-yl]-3-[(3-methyl-1,2-thiazol-5-yl)amino]pyrazine-2-carboxamide (D-159) was prepared using 5-[(2R,3R)-3-amino-2-methylpiperidin-1-yl]-3-[(3-methyl-1,2-thiazol-5-yl)amino]pyrazine-2-carboxamide. MS found for C25H31N7O3S as (M+H)⁺ 510.08.

¹H NMR (400 MHz, DMSO) δ 12.28 (s, 1H), 8.36 (d, J=7.53 Hz, 1H), 7.94-7.87 (m, 1H), 7.86-7.79 (m, 3H), 7.60-7.51 (m, 3H), 6.83 (s, 1H), 5.11 (s, 2H), 4.60-3.78 (m, 2H), 3.24-3.11 (m, 1H), 2.27 (s, 3H), 2.05-1.58 (m, 4H), 1.45 (s, 6H), 1.22 (d, J=6.78 Hz, 3H).

### Example D-160: Synthesis of 5-[(2R,3R)-3-[3-fluoro-4-(2-hydroxypropan-2-yl)benzamido]-2-methylpiperidin-1-yl]-3-[(1-methyl-1H-pyrazol-4-yl)amino]pyrazine-2-carboxamide (D-160)

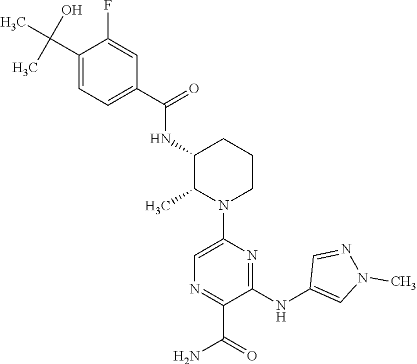 [SMILES: C[C@@H]1[C@H](NC(=O)c2ccc(C(C)(C)O)c(F)c2)CCCN1c1cnc(C(N)=O)c(Nc2cnn(C)c2)n1]

In a similar manner as described in Example D-216 5-[(2R,3R)-3-[3-fluoro-4-(2-hydroxypropan-2-yl)benzamido]-2-methylpiperidin-1-yl]-3-[(1-methyl-1H-pyrazol-4-yl)amino]pyrazine-2-carboxamide (D-160) was prepared using 5-[(2R,3R)-3-amino-2-methylpiperidin-1-yl]-3-[(1-methyl-1H-pyrazol-4-yl)amino]pyrazine-2-carboxamide. MS found for C25H31 FN8O3 as (M+H)⁺ 511.16.

¹H NMR (500 MHz, DMSO) δ 10.87 (s, 1H), 8.46 (d, J=6.59 Hz, 1H), 8.01 (br. s., 1H), 7.81-7.60 (m, 4H), 7.56 (s, 1H), 7.48 (s, 1H), 7.28 (br. s., 1H), 5.40 (s, 2H), 4.22-3.95 (m, 2H), 3.77 (s, 3H), 3.08 (t, J=12.21 Hz, 1H), 2.05-1.55 (m, 4H), 1.50 (s, 6H), 1.09 (d, J=6.86 Hz, 3H).

### Example D-161: Synthesis of 5-[(2R,3R)-3-[4-(2-methoxypropan-2-yl)benzamido-2-methylpiperidin-1-yl]-3-(1-methyl-1H-pyrazol-4-yl)amino]pyrazine-2-carboxamide (D-161)

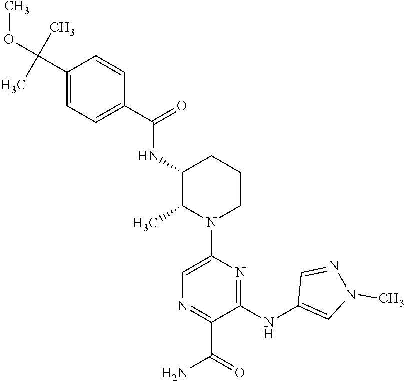 [SMILES: COC(C)(C)c1ccc(C(=O)N[C@@H]2CCCN(c3cnc(C(N)=O)c(Nc4cnn(C)c4)n3)[C@@H]2C)cc1]

In a similar manner as described in Example D-216 5-[(2R,3R)-3-[4-(2-methoxypropan-2-yl)benzamido]-2-methylpiperidin-1-yl]-3-[(1-methyl-1H-pyrazol-4-yl)amino]pyrazine-2-carboxamide (D-161) was prepared using 5-[(2R,3R)-3-amino-2-methylpiperidin-1-yl]-3-[(1-methyl-1H-pyrazol-4-yl)amino]pyrazine-2-carboxamide. MS found for C26H34N8O3 as (M+H)⁺ 507.19.

¹H NMR (500 MHz, DMSO) δ 10.88 (s, 1H), 8.42 (d, J=6.36 Hz, 1H), 8.03 (br. s., 1H), 7.90 (d, J=8.31 Hz, 2H), 7.70 (br. s., 1H), 7.57 (s, 1H), 7.53-7.48 (m, 3H), 7.28 (br. s., 1H), 5.57-5.05 (m, 1H), 4.27-3.98 (m, 2H), 3.77 (s, 3H), 3.09 (t, J=12.96 Hz, 1H), 3.00 (s, 3H), 2.03-1.54 (m, 4H), 1.47 (s, 6H), 1.10 (d, J=6.36 Hz, 3H).

### Example D-162: Synthesis of 5-[(2R,3R)-3-[(dimethylcarbamoyl)amino]-2-methylpiperidin-1-yl]-3-{[3-(oxan-4-yl)-1,2-thiazol-5-yl]amino}pyrazine-2-carboxamide (D-162)

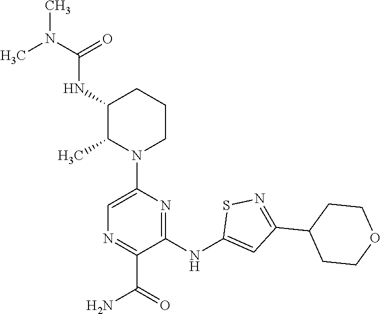 [SMILES: C[C@@H]1[C@H](NC(=O)N(C)C)CCCN1c1cnc(C(N)=O)c(Nc2cc(C3CCOCC3)ns2)n1]

In a similar manner as described in Example 59, 5-[(2R,3R)-3-[(dimethylcarbamoyl)amino]-2-methylpiperidin-1-yl]-3-{[3-(oxan-4-yl)-1,2-thiazol-5-yl]amino}pyrazine-2-carboxamide (D-162) was prepared using 1-[(2R,3R)-1-(6-chloro-5-cyanopyrazin-2-yl)-2-methylpiperidin-3-yl]-3,3-dimethylurea. MS found for C22H32N8O3S as (M+H)⁺ 489.10.

¹H NMR (500 MHz, DMSO) δ 12.29 (s, 1H), 7.90 (br. s., 1H), 7.77 (s, 1H), 7.55 (br. s., 1H), 6.96 (s, 1H), 6.14 (d, J=6.85 Hz, 1H), 5.14-4.25 (m, 2H), 3.90 (dd, J=11.25, 1.96 Hz, 2H), 3.78-3.62 (m, 1H), 3.50-3.37 (m, 2H), 3.11 (t, J=12.23 Hz, 1H), 2.94-2.78 (m, 7H), 1.91-1.51 (m, 8H), 1.17 (d, J=6.85 Hz, 3H).

### Example D-163: Synthesis of 3-{[3-(1-cyclopentylpiperidin-4-yl)-1,2-thiazol-5-yl]amino}-5-[(2R,3R)-3-[(dimethylcarbamoyl)amino]-2-methylpiperidin-1-yl]pyrazine-2-carboxamide (D-163)

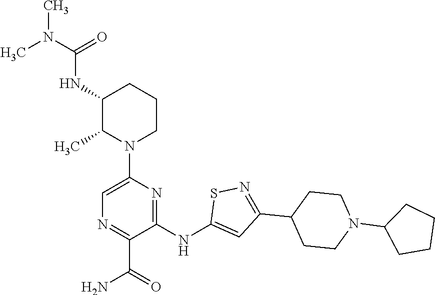 [SMILES: C[C@@H]1[C@H](NC(=O)N(C)C)CCCN1c1cnc(C(N)=O)c(Nc2cc(C3CCN(C4CCCC4)CC3)ns2)n1]

In a similar manner as described in Example 59, 3-{[3-(1-cyclopentylpiperidin-4-yl)-1,2-thiazol-5-yl]amino}-5-[(2R,3R)-3-[(dimethylcarbamoyl)amino]-2-methylpiperidin-1-yl]pyrazine-2-carboxamide (D-163) was prepared using 1-[(2R,3R)-1-(6-chloro-5-cyanopyrazin-2-yl)-2-methylpiperidin-3-yl]-3,3-dimethylurea. MS found for C27H41N9O2S as (M+H)⁺ 556.26.

¹H NMR (400 MHz, DMSO) δ 12.27 (s, 1H), 7.88 (s, 1H), 7.77 (s, 1H), 7.53 (br. s., 1H), 6.93 (s, 1H), 6.13 (d, J=7.24 Hz, 1H), 5.14-4.36 (m, 2H), 3.72 (d, J=4.60 Hz, 1H), 3.17-2.93 (m, 3H), 2.65-2.56 (m, 2H), 2.20-1.29 (m, 20H), 1.21-1.12 (m, 3H).

### Example D-164: Synthesis of 3-[(3-methyl-1,2-thiazol-5-yl)amino]-5-[3-(4-phenyl-3H-imidazol-2-yl)piperidin-1-yl]pyrazine-2-carboxamide (D-164)

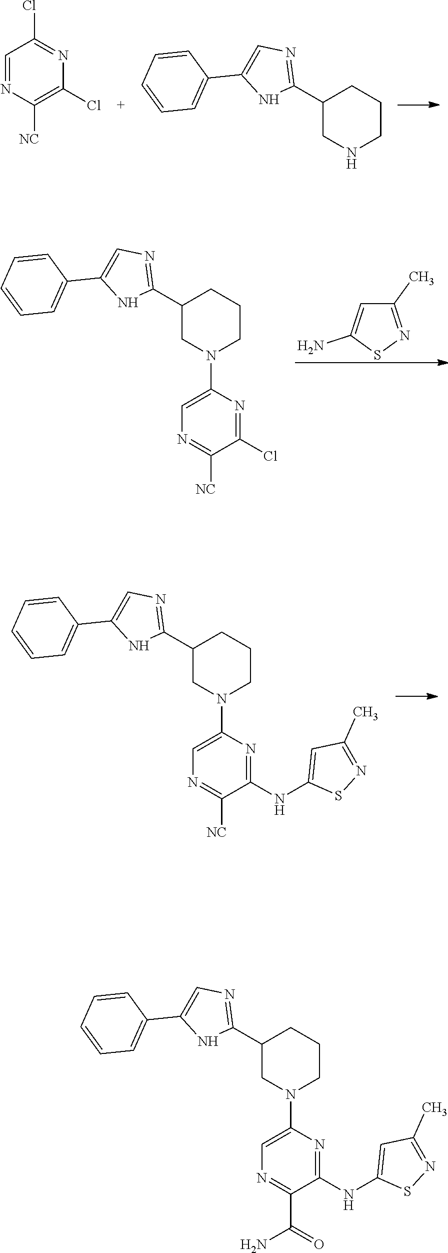

3,5-Dichloropyrazine-2-carbonitrile (275.0 mg, 1.58 mmol) and 3-(4-phenyl-3H-imidazol-2-yl)piperidine (300.0 mg, 1.32 mmol) were dissolved in DMF (5 mL), DIPEA (460.0 μL, 2.64 mmol) was added and the mixture was heated at 60° C. for 2 h. DMF was evaporated and the residue purified by silica flash chromatography with 20 to 100% ethyl acetate in cyclohexane to give 3-chloro-5-[3-(4-phenyl-3H-imidazol-2-yl)piperidin-1-yl]pyrazine-2-carbonitrile (270.0 mg, 47% yield) as a white solid. MS found for C19H17ClN6 as (M+H)⁺ 365.3.

3-chloro-5-[3-(4-phenyl-3H-imidazol-2-yl)piperidin-1-yl]pyrazine-2-carbonitrile (270.0 mg, 0.74 mmol), 3-methyl-1,2-thiazol-5-amine hydrochloride (102.0 mg, 0.89 mmol), and Cs₂CO₃ (723.0 mg, 2.22 mmol) were suspended in dioxane (10 mL). (+/−) BINAP (93.4 mg, 0.15 mmol) and Pd(OAc)₂ (33.7 mg, 0.15 mmol) were added under nitrogen and the mixture stirred for 2 h at 90° C. The insoluble material was filtered off and the filtrate concentrated. The residue was purified by silica flash chromatography with 20 to 100% ethyl acetate in cyclohexane to give 3-chloro-5-[3-(4-phenyl-3H-imidazol-2-yl)piperidin-1-yl]pyrazine-2-carbonitrile (100.0 mg, 34% yield) as a yellow solid. MS found for C23H22N8S as (M+H)⁺ 443.0.

3-Chloro-5-[3-(4-phenyl-3H-imidazol-2-yl)piperidin-1-yl]pyrazine-2-carbonitrile (100.0 mg, 0.23 mmol) was dissolved in TFA (2 mL), H₂SO₄ (200 μl) was added. The mixture was stirred at 50° C. for 2 h. The solvent was evaporated and the residue taken up with DCM and washed with NaHCO₃ sat. aqueous solution. The organic phase was separated, dried over Na₂SO₄ and concentrated. The residue was purified by silica NH flash chromatography with 0 to 10% MeOH in DCM. The product was then purified by preparative HPLC to give 3-[(3-methyl-1,2-thiazol-5-yl)amino]-5-[3-(4-phenyl-3H-imidazol-2-yl)piperidin-1-yl]pyrazine-2-carboxamide (D-164) (10.0 mg, 9% yield) as a white solid. MS found for C23H24N8OS as (M+H)⁺ 461.0.

¹H NMR (500 MHz, DMSO) δ 12.29 (s, 1H), 12.19-11.74 (m, 1H), 7.92 (s, 1H), 7.90-7.88 (m, 1H), 7.81-7.65 (m, 2H), 7.61-7.42 (m, 2H), 7.33 (t, J=7.41 Hz, 2H), 7.19-7.13 (m, 1H), 6.84 (s, 1H), 4.94-4.31 (m, 2H), 3.70-3.21 (m, 2H), 3.09-2.92 (m, 1H), 2.28 (s, 3H), 2.21-2.10 (m, 1H), 2.03-1.84 (m, 2H), 1.74-1.58 (m, 1H).

### Example D-165: Synthesis of 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyrazine-2-carboxamide (D-165)

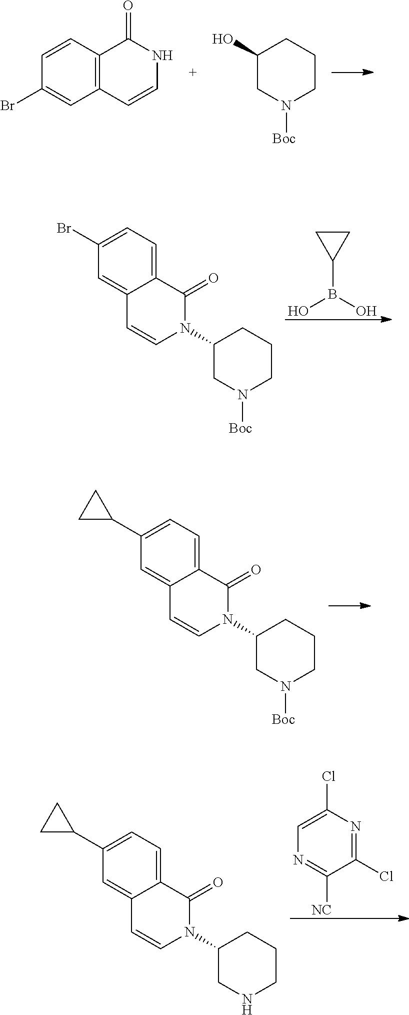

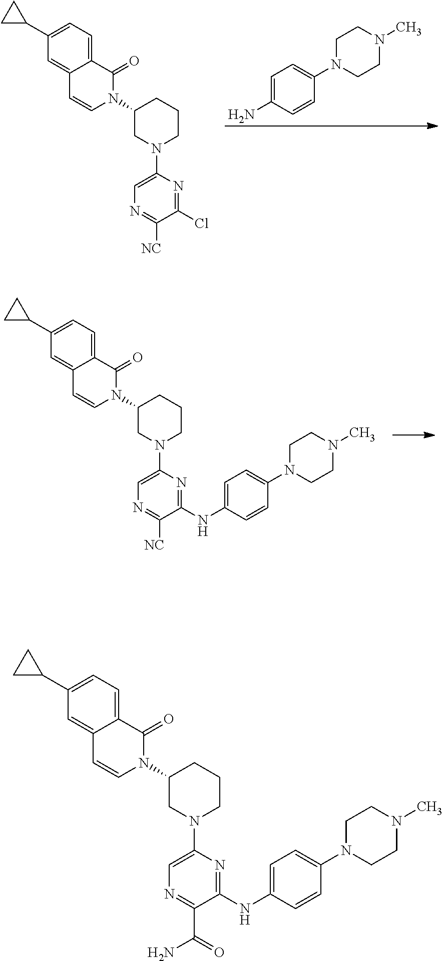 [SMILES: N#Cc1ncc(N2CCC[C@@H](n3ccc4cc(C5CC5)ccc4c3=O)C2)nc1Cl ; CN1CCN(c2ccc(N)cc2)CC1 ; CN1CCN(c2ccc(Nc3nc(N4CCC[C@@H](n5ccc6cc(C7CC7)ccc6c5=O)C4)cnc3C#N)cc2)CC1 ; CN1CCN(c2ccc(Nc3nc(N4CCC[C@@H](n5ccc6cc(C7CC7)ccc6c5=O)C4)cnc3C(N)=O)cc2)CC1]

To a solution of 6-bromo-2H-isoquinolin-1-one (2 g, 8.93 mmol) in THF (40 mL), tert-butyl (3S)-3-hydroxypiperidine-1-carboxylate (2.7 g, 13.39 mmol) and Ph₃P (5.8 g, 22.3 mmol) were added followed by the addition of DIAD (4.4 mL, 22.3 mmol) dropwise. The mixture was stirred at room temperature for 10 h then it was concentrated. Ethyl acetate, H₂O and brine were added. The organic phase was dried over Na₂SO₄, concentrated and purified by silica flash chromatography with 0 to 10% ethyl acetate in DCM. The product, obtained as yellow oil was repurified by C18 reverse phase silica flash chromatography with 0 to 100/ACN in H₂O to give tert-butyl (3R)-3-(6-bromo-1-oxoisoquinolin-2-yl)piperidine-1-carboxylate (197 mg, 5% yield) as white foam. MS found for C19H23BrN2O3 as (M+H)⁺ 406.9, 408.9.

To a solution of tert-butyl (3R)-3-(6-bromo-1-oxoisoquinolin-2-yl)piperidine-1-carboxylate (197.0 mg, 0.48 mmol) in toluene/H₂O (4 mL/1 mL), K₃PO₄ (204.0 mg, 0.96 mmol), cyclopropylboronic acid (48.3, 0.56 mmol), PCy₃ (15.4 mg, 0.055 mmol) and Pd(OAc)₂ (6.2 mg, 0.027 mmol) were added. The mixture was stirred at 100° C. for 1 day, then left to reach room temperature, concentrated and taken up with ethyl acetate. The organic phase was separated to give a crude that was purified by silica flash chromatography with 10 to 40% ethyl acetate in cyclohexane to give tert-butyl (3R)-3-(6-cyclopropyl-1-oxoisoquinolin-2-yl)piperidine-1-carboxylate (131.0 mg, 74% yield) as a white foam. MS found for C₂₂H₂₈N₂O₃ as (M+H)⁺ 369.0.

To a solution of tert-butyl (3R)-3-(6-cyclopropyl-1-oxoisoquinolin-2-yl)piperidine-1-carboxylate (131.0 mg, 0.35 mmol) in DCM (5 mL), 4N HCl in dioxane (0.9 mL, 3.5 mmol) was added. The mixture was stirred at room temperature for 2 h then it was evaporated to dryness to give 6-cyclopropyl-2-[(3R)-piperidin-3-yl]isoquinolin-1-one hydrochloride (111.0 mg, quant. yield) as a white solid. MS found for C₁₇H₂₀N₂O as (M+H)⁺ 269.2.

To a solution of 6-cyclopropyl-2-[(3R)-piperidin-3-yl]isoquinolin-1-one hydrochloride (111.0 mg, 0.36 mmol) in DMF (2 mL), DIPEA (0.2 mL, 1.08 mmol) and 3,5-dichloropyrazine-2-carbonitrile (68.9 mg, 0.39 mmol) were added. The mixture was stirred at room temperature for 2 h, then it was concentrated and purified by silica flash chromatography with 20 to 40% ethyl acetate in cyclohexane to give 3-chloro-5-[(3R)-3-(6-cyclopropyl-1-oxoisoquinolin-2-yl)piperidin-1-yl]pyrazine-2-carbonitrile (138.0 mg, 94% yield) as a pink solid. MS found for C22H20ClN5O as (M+H)⁺ 406.3.

To a solution of 3-chloro-5-[(3R)-3-(6-cyclopropyl-1-oxoisoquinolin-2-yl)piperidin-1-yl]pyrazine-2-carbonitrile (69.0 mg, 0.17 mmol) in 1,4-dioxane (3 mL), Cs₂CO₃ (225.0 mg, 0.69 mmol), 4-(4-methylpiperazin-1-yl)aniline (66.0 mg, 0.34 mmol), (+/−) BINAP (22.0 mg, 0.034 mmol) and Pd(OAc)₂ (8.0 mg, 0.034 mmol) were added and the mixture stirred for 2 h at 90° C., then it was left to cool to room temperature. The mixture was partitioned between H₂O and ethyl acetate. The combined organic phases were purified by silica NH flash chromatography with 0 to 5% MeOH in DCM to give 5-[(3R)-3-(6-cyclopropyl-1-oxoisoquinolin-2-yl)piperidin-1-yl]-3-{[4-(4-methylpiperazin-1-yl)phenyl]amino}pyrazine-2-carbonitrile (56.0 mg, 59% yield) as a yellow solid. MS found for C₃₃H₃₆N₈O as (M+H)⁺ 561.5.

To a suspension of 5-[(3R)-3-(6-cyclopropyl-1-oxoisoquinolin-2-yl)piperidin-1-yl]-3-{[4-(4-methylpiperazin-1-yl)phenyl]amino}pyrazine-2-carbonitrile (56 mg, 0.099 mmol), in MeOH/DMSO (2 mL/0.2 mL), TEA (0.9 mL), NaOH (11.3 mg, 0.28 mmol) and H₂O₂ (0.1 mL) were added. The mixture was stirred overnight at room temperature, and then it was partitioned between DCM and H₂O. The combined organic phases were dried over Na₂SO₄ and concentrated. The crude was purified by silica NH flash chromatography with 50 to 100% ethyl acetate in cyclohexane to give 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyrazine-2-carboxamide (D-165) (33.8 mg, 59% yield) as a yellow solid. MS found for C33H38N8O2 as (M+H)⁺ 579.5.

¹H NMR (500 MHz, DMSO) δ 10.78 (br. s., 1H), 8.22 (d, J=8.50 Hz, 1H), 7.75-7.66 (m, 2H), 7.60-7.54 (m, 1H), 7.42-7.37 (m, 1H), 7.36-7.27 (m, 3H), 7.25 (dd, J=8.50, 1.60 Hz, 1H), 6.63 (d, J=7.58 Hz, 1H), 6.57-6.42 (m, 2H), 5.00-4.87 (m, 1H), 4.56 (d, J=11.00 Hz, 1H), 4.34 (d, J=12.96 Hz, 1H), 3.26-3.07 (m, 2H), 2.80-2.56 (m, 4H), 2.33-2.04 (m, 9H), 2.02-1.85 (m, 2H), 1.79-1.61 (m, 1H), 1.14-1.05 (m, 2H), 0.91-0.76 (m, 2H).

### Example D-166: Synthesis of 5-[(3R)-3-(6-cyclopropyl-1-oxo-1,2-dihydroisoquinolin-2-yl)piperidin-1-yl]-3-[(3-methyl-1,2-thiazol-5-yl)amino]pyrazine-2-carboxamide (D-166)

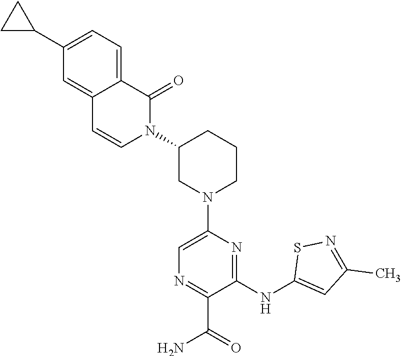 [SMILES: Cc1cc(Nc2nc(N3CCC[C@@H](n4ccc5cc(C6CC6)ccc5c4=O)C3)cnc2C(N)=O)sn1]

In a similar manner as described in Example D-165, 5-[(3R)-3-(6-cyclopropyl-1-oxo-1,2-dihydroisoquinolin-2-yl)piperidin-1-yl]-3-[(3-methyl-1,2-thiazol-5-yl)amino]pyrazine-2-carboxamide (D-166) was prepared. MS found for C26H27N7O2S as (M+H)⁺ 502.4.

¹H NMR (500 MHz, DMSO) δ 12.29 (s, 1H), 8.09 (d, J=8.37 Hz, 1H), 7.94 (s, 1H), 7.91 (s, 1H), 7.62-7.55 (m, 2H), 7.35 (d, J=1.65 Hz, 1H), 7.21 (dd, J=8.44, 1.60 Hz, 1H), 6.84 (s, 1H), 6.62 (d, J=7.55 Hz, 1H), 4.92-4.80 (m, 1H), 4.64 (br. s., 2H), 3.53 (t, J=12.01 Hz, 1H), 3.23-3.13 (m, 1H), 2.29-2.13 (m, 4H), 2.09-2.02 (m, 1H), 2.01-1.90 (m, 2H), 1.80-1.67 (m, 1H), 1.10-1.03 (m, 2H), 0.86-0.77 (m, 2H).

### Preparation of 4-[(4-methylpiperazin-1-yl)methyl]aniline

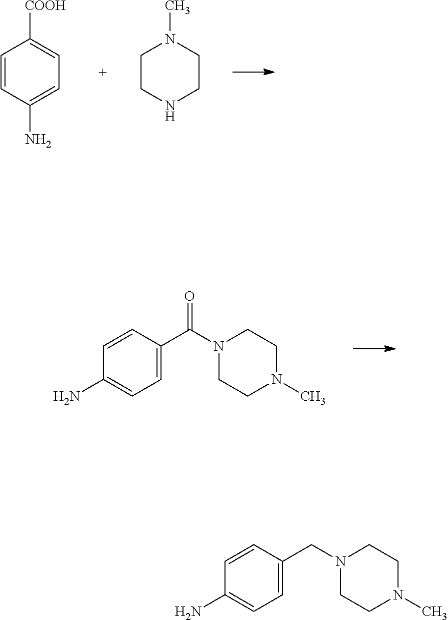

To a mixture of 4-aminobenzoic acid (5 g, 36.5 mmol), 1-methylpiperazine (3.7 mL, 32.8 mmol) and TEA (16.0 mL, 114.8 mmol) in DCM (100 mL) was added EDC.HCl (10.5 g, 54.8 mmol) and the mixture was stirred at room temperature overnight. The solvent was removed and the residue was purified by silica flash chromatography with 0 to 10% MeOH in DCM. The light yellow oil obtained was dissolved in DCM and filtered. The yellow solid obtained was dissolved in DCM washed with brine then with Na₂CO₃ 2M. The organic phase was dried and evaporated to give 4-(4-methylpiperazine-1-carbonyl)aniline (3 g, 42% yield). MS found for C12H17N3O as (M+H)⁺ 220.2.

A solution of 4-(4-methylpiperazine-1-carbonyl)aniline (1.03 g, 4.56 mmol) in THF (50 mL) was cooled to 0° C. and LiAlH₄ 1 M in THF (14 mL, 14 mmol) was added dropwise. The mixture was let to warm to room temperature, and then refluxed for 3 h. After cooling to 0° C. Na₂SO₄.10H₂O was added and the solution was filtered off. The solution was concentrated and purified by silica flash chromatography with 0 to 5% MeOH in DCM to give 4-[(4-methylpiperazin-1-yl)methyl]aniline (500 mg, 53% yield). MS found for C₁₂H₁₉N₃ as (M+H)⁺ 206.3.

### Example D-167: Synthesis of 5-[(3R)-3-(6-cyclopropyl-1-oxo-1,2-dihydroisoquinolin-2-yl)piperidin-1-yl]-3-({4-[(4-methylpiperazin-1-yl)methyl]phenyl}amino)pyrazine-2-carboxamide (D-167)

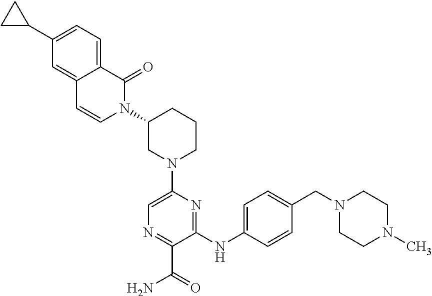 [SMILES: CN1CCN(Cc2ccc(Nc3nc(N4CCC[C@@H](n5ccc6cc(C7CC7)ccc6c5=O)C4)cnc3C(N)=O)cc2)CC1]

In a similar manner as described in Example D-165, 5-[(3R)-3-(6-cyclopropyl-1-oxo-1,2-dihydroisoquinolin-2-yl)piperidin-1-yl]-3-({4-[(4-methylpiperazin-1-yl)methyl]phenyl}amino)pyrazine-2-carboxamide (D-167) was prepared. MS found for C34H40N8O2 as (M+H)⁺ 593.6.

¹H NMR (400 MHz, DMSO) δ 11.20 (s, 1H), 8.18 (d, J=8.33 Hz, 1H), 7.79 (br. s., 1H), 7.75 (s, 1H), 7.58 (d, J=7.45 Hz, 1H), 7.47 (d, J=8.33 Hz, 2H), 7.37 (s, 2H), 7.24 (dd, J=8.33, 1.75 Hz, 1H), 7.00 (d, J=7.45 Hz, 2H), 6.62 (d, J=7.45 Hz, 1H), 4.90 (t, J=11.40 Hz, 1H), 4.54 (d, J=12.72 Hz, 1H), 4.40 (d, J=12.28 Hz, 1H), 3.29-3.05 (m, 4H), 2.11 (s, 12H), 1.99-1.90 (m, 2H), 1.79-1.59 (m, 1H), 1.12-1.03 (m, 2H), 0.87-0.77 (m, 2H).

### Example D-168: Synthesis of 5-[(3R)-3-(6-cyclopropyl-1-oxo-1,2-dihydroisoquinolin-2-yl)piperidin-1-yl]-3-[(quinolin-6-yl)amino]pyrazine-2-carboxamide (D-168)

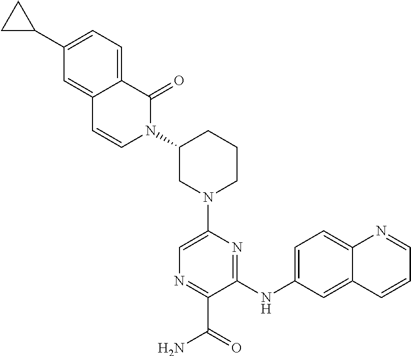 [SMILES: NC(=O)c1ncc(N2CCC[C@@H](n3ccc4cc(C5CC5)ccc4c3=O)C2)nc1Nc1ccc2ncccc2c1]

In a similar manner as described in Example D-165, 5-[(3R)-3-(6-cyclopropyl-1-oxo-1,2-dihydroisoquinolin-2-yl)piperidin-1-yl]-3-[(quinolin-6-yl)amino]pyrazine-2-carboxamide (D-168) was prepared. MS found for C₃₁H₂₉N₇O₂ as (M+H)⁺ 532.5.

¹H NMR (400 MHz, DMSO) δ 11.71 (s, 1H), 8.60 (d, J=3.07 Hz, 1H), 8.34 (d, J=2.19 Hz, 1H), 8.15 (d, J=8.33 Hz, 1H), 7.93-7.88 (m, 1H), 7.87-7.85 (m, 1H), 7.82 (s, 1H), 7.78-7.66 (m, 2H), 7.64 (d, J=7.45 Hz, 1H), 7.49 (br. s., 1H), 7.41 (d, J=1.32 Hz, 1H), 7.29 (dd, J=8.55, 1.53 Hz, 1H), 6.90 (br. s., 1H), 6.66 (d, J=7.45 Hz, 1H), 5.00 (t, J=11.62 Hz, 1H), 4.67 (d, J=10.96 Hz, 1H), 4.45 (d, J=11.84 Hz, 1H), 3.38-3.32 (m, 1H), 3.15 (t, J=12.06 Hz, 1H), 2.21 (dd, J=12.06, 3.73 Hz, 1H), 2.15-2.06 (m, 1H), 1.99 (br. s., 2H), 1.78 (d, J=13.15 Hz, 1H), 1.19-1.03 (m, 2H), 0.93-0.77 (m, 2H).

### Example D-169: Synthesis of 5-[(3R)-3-(6-cyclopropyl-1-oxo-1,2-dihydroisoquinolin-2-yl)piperidin-1-yl]-3-{[4-(4-methylpiperazine-1-carbonyl)phenyl]amino}pyrazine-2-carboxamide (D-169)

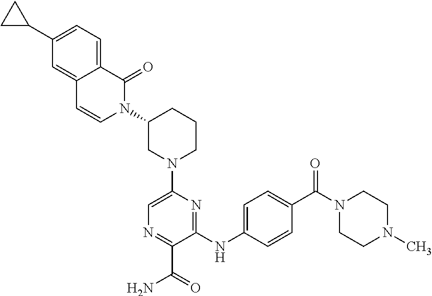 [SMILES: CN1CCN(C(=O)c2ccc(Nc3nc(N4CCC[C@@H](n5ccc6cc(C7CC7)ccc6c5=O)C4)cnc3C(N)=O)cc2)CC1]

In a similar manner as described in Example D-165, 5-[(3R)-3-(6-cyclopropyl-1-oxo-1,2-dihydroisoquinolin-2-yl)piperidin-1-yl]-3-{[4-(4-methylpiperazine-1-carbonyl)phenyl]amino}pyrazine-2-carboxamide (D-169) was prepared. MS found for C34H38N8O3 as (M+H)⁺ 607.6.

¹H NMR (400 MHz, DMSO) δ 11.45 (s, 1H), 8.15 (d, J=8.28 Hz, 1H), 7.89-7.77 (m, 2H), 7.67-7.55 (m, 3H), 7.43 (br. s., 1H), 7.37 (d, J=1.38 Hz, 1H), 7.23 (dd, J=8.30, 1.40 Hz, 1H), 7.16 (d, J=8.03 Hz, 2H), 6.62 (d, J=7.53 Hz, 1H), 5.02-4.84 (m, 1H), 4.56 (d, J=12.30 Hz, 1H), 4.42 (d, J=10.42 Hz, 1H), 3.43-3.23 (m, 1H), 3.20-3.06 (m, 1H), 2.30-1.87 (m, 15H), 1.81-1.64 (m, 1H), 1.13-0.99 (m, 2H), 0.91-0.73 (m, 2H).

### Example D-170: Synthesis of 5-[(2R,3R)-3-{2-fluoro-4-[(1E)-prop-1-en-1-yl]benzamido}-2-methylpiperidin-1-yl]-3-[(3-methyl-1,2-thiazol-5-yl)amino]pyridine-2-carboxamide (D-170)

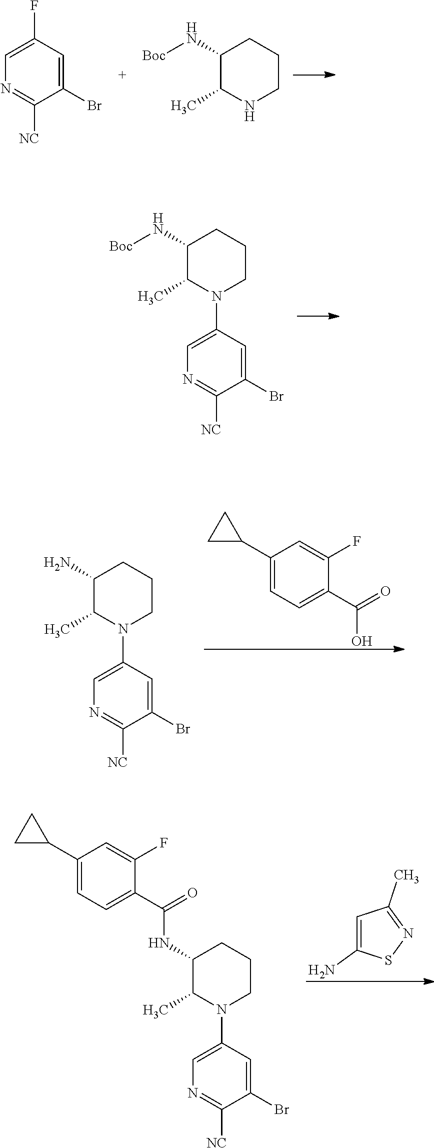

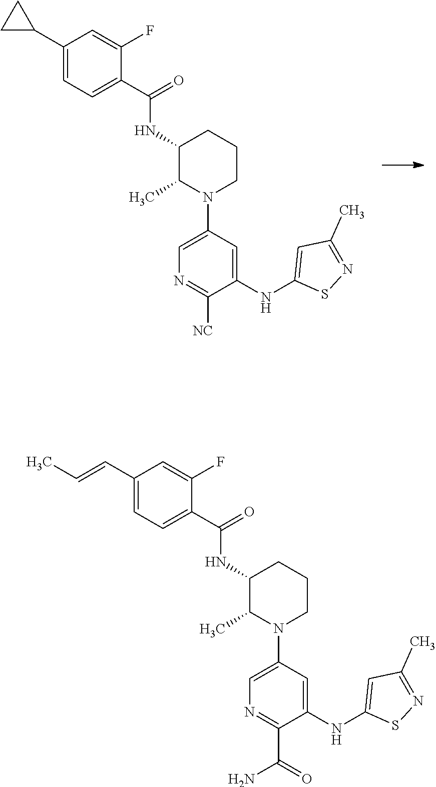 [SMILES: Cc1cc(Nc2cc(N3CCC[C@@H](NC(=O)c4ccc(C5CC5)cc4F)[C@H]3C)cnc2C#N)sn1 ; C/C=C/c1ccc(C(=O)N[C@@H]2CCCN(c3cnc(C(N)=O)c(Nc4cc(C)ns4)c3)[C@@H]2C)c(F)c1]

3-Bromo-5-fluoropyridine-2-carbonitrile (400.0 mg, 1.99 mmol), tert-butyl N-[(2R,3R)-2-methylpiperidin-3-yl]carbamate (427.0 mg, 1.99 mmol) and DIPEA (700.0 μL, 3.98 mmol) were dissolved in DMF (8 mL), the mixture was stirred for 2 h at 90° C. 3-Bromo-5-fluoropyridine-2-carbonitrile (200.0 mg, 1.0 mmol) was added and the reaction stirred for 2 h at 90° C. DMF was evaporated and the product purified by silica flash chromatography with 10 to 100% ethyl acetate in cyclohexane to give tert-butyl N-[(2R,3R)-1-(5-bromo-6-cyanopyridin-3-yl)-2-methylpiperidin-3-yl]carbamate (630.0 mg, 80 yield). MS found for C17H23BrN4O2 as (M+H)⁺ 395.2, 397.2.

Tert-butyl N-[(2R,3R)-1-(5-bromo-6-cyanopyridin-3-yl)-2-methylpiperidin-3-yl]carbamate (630.0 mg, 1.59 mmol) was dissolved in HCl in MeOH 1.25 N (5.1 mL, 6.37 mmol) and stirred at room temperature for 4 h. The solvent was evaporated to give a white solid which was passed through an SCX cartridge. Evaporation of the ammonia fractions gave 5-[(2R,3R)-3-amino-2-methylpiperidin-1-yl]-3-bromopyridine-2-carbonitrile (300.0 mg, 64% yield). MS found for C12H15BrN4 as (M+H)⁺ 295.0, 297.0.

4-Cyclopropyl-2-fluorobenzoic acid (300.0 mg, 1.02 mmol) was dissolved in DCM (5 mL), (COCl)₂ (863 μL, 1.02 mmol) was added, followed by a drop of DMF. Et₃N (142 μL, 1.02 mmol) was added and the mixture stirred at 50° C. for 4 h. 5-[(2R,3R)-3-amino-2-methylpiperidin-1-yl]-3-bromopyridine-2-carbonitrile (300.0 mg, 1.02 mmol) was added and the mixture stirred at room temperature overnight. The solvent was evaporated and the residue purified by silica flash chromatography with 20 to 100% ethyl acetate in cyclohexane to give N-[(2R,3R)-1-(5-bromo-6-cyanopyridin-3-yl)-2-methylpiperidin-3-yl]-4-cyclopropyl-2-fluorobenzamide (150.0 mg, 32% yield). MS found for C22H22BrFN4O as (M+H)⁺ 457.0, 459.0.

N-[(2R,3R)-1-(5-bromo-6-cyanopyridin-3-yl)-2-methylpiperidin-3-yl]-4-cyclopropyl-2-fluorobenzamide (150.0 mg, 0.33 mmol), 3-methyl-1,2-thiazol-5-amine hydrochloride (75.0 mg, 0.50 mmol), and Cs₂CO₃ (430.0 mg, 1.32 mmol) were suspended in dioxane (4 mL), Pd(OAc)₂ (15.0 mg, 0.066 mmol) and (+/−) BINAP (41.0 mg, 0.066) were added while degassing with nitrogen. The mixture was heated at 90° C. overnight for 4 h. The solvent was evaporated and the residue purified by silica flash chromatography with 0 to 20% MeOH in DCM to give N-[(2R,3R)-1-(6-cyano-5-[(3-methyl-1,2-thiazol-5-yl)amino]pyridin-3-yl)-2-methylpiperidin-3-yl]-4-cyclopropyl-2-fluorobenzamide (50.0 mg, 31% yield). MS found for C26H27FN6OS as (M+H)⁺ 491.13.

N-[(2R,3R)-1-(6-cyano-5-[(3-methyl-1,2-thiazol-5-yl)amino]pyridin-3-yl)-2-methylpiperidin-3-yl]-4-cyclopropyl-2-fluorobenzamide (50.0 mg, 0.10 mmol) was dissolved in H₂SO₄ (200.0 μL) and TFA (2 mL). The mixture was stirred at room temperature for 4 h. TFA was evaporated and the residue treated with DCM then washed with NaHCO₃ aq. solution. The DCM phase was concentrated and the residue purified by silica flash chromatography with 0 to 30% MeOH in DCM. The product, obtained was repurified by C18 reverse phase silica flash chromatography with 5 to 100%0/ACN in H₂O 0.1% HCOOH to give 5-[(2R,3R)-3-{2-fluoro-4-[(1E)-prop-1-en-1-yl]benzamido}-2-methylpiperidin-1-yl]-3-[(3-methyl-1,2-thiazol-5-yl)amino]pyridine-2-carboxamide (D-170) (9.0 mg, 17% yield) as white solid. MS found for C26H29FN6O2S as (M+H)⁺ 509.1.

¹H NMR (400 MHz, DMSO) δ 12.02 (s, 1H), 8.34 (d, J=7.03 Hz, 1H), 8.02 (d, J=2.38 Hz, 1H), 7.91 (d, J=2.30 Hz, 1H), 7.57 (d, J=2.40 Hz, 1H), 7.51 (t, J=7.78 Hz, 1H), 7.36-7.25 (m, 2H), 6.93 (d, J=2.26 Hz, 1H), 6.85 (s, 1H), 6.57-6.41 (m, 2H), 4.50 (quin, J=6.15 Hz, 1H), 4.19-3.97 (m, 1H), 3.70 (d, J=11.92 Hz, 1H), 3.07 (t, J=1.90 Hz, 1H), 2.31 (s, 3H), 1.93-1.75 (m, 5H), 1.74-1.58 (m, 2H), 1.13 (d, J=6.10 Hz, 3H).

### Example D-171: Synthesis of 3-[(3-methyl-1,2-thiazol-5-yl)amino]-5-[(3R)-3-(5-phenyl-1H-imidazol-2-yl)piperidin-1-yl]pyrazine-2-carboxamide (D-171)

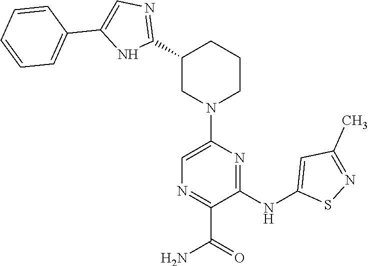 [SMILES: Cc1cc(Nc2nc(N3CCC[C@@H](c4ncc(-c5ccccc5)[nH]4)C3)cnc2C(N)=O)sn1]

Chiral separation of 3-[(3-methyl-1,2-thiazol-5-yl)amino]-5-[3-(4-phenyl-3H-imidazol-2-yl)piperidin-1-yl]pyrazine-2-carboxamide (D-164) afforded 3-[(3-methyl-1,2-thiazol-5-yl)amino]-5-[(3R)-3-(5-phenyl-1H-imidazol-2-yl)piperidin-1-yl]pyrazine-2-carboxamide (D-171). MS found for C23H24N8OS as (M+H)⁺ 461.1.

¹H NMR (400 MHz, DMSO) δ 12.29 (s, 1H), 11.97 (br. s., 1H), 7.97-7.86 (m, 2H), 7.77 (d, J=7.04 Hz, 2H), 7.54 (d, J=1.96 Hz, 2H), 7.31 (t, J=7.63 Hz, 2H), 7.25-7.08 (m, 1H), 6.84 (s, 1H), 5.00-4.34 (m, 2H), 3.59-3.16 (m, 2H), 3.00 (t, J=10.76 Hz, 1H), 2.28 (s, 3H), 2.15 (d, J=12.91 Hz, 1H), 2.05-1.79 (m, 2H), 1.66 (d, J=12.13 Hz, 1H).

### Example D-172: Synthesis of 3-[(3-methyl-1,2-thiazol-5-yl)amino]-5-[(3S)-3-(5-phenyl-1H-imidazol-2-yl)piperidin-1-yl]pyrazine-2-carboxamide (D-172)

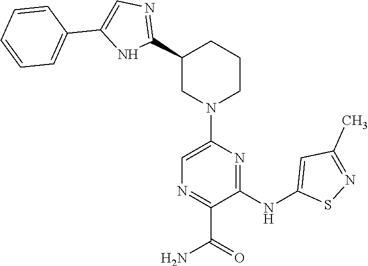 [SMILES: Cc1cc(Nc2nc(N3CCC[C@H](c4ncc(-c5ccccc5)[nH]4)C3)cnc2C(N)=O)sn1]

Chiral separation of 3-[(3-methyl-1,2-thiazol-5-yl)amino]-5-[3-(4-phenyl-3H-imidazol-2-yl)piperidin-1-yl]pyrazine-2-carboxamide (D-164) afforded 3-[(3-methyl-1,2-thiazol-5-yl)amino]-5-[(3S)-3-(5-phenyl-1H-imidazol-2-yl)piperidin-1-yl]pyrazine-2-carboxamide (D-172). MS found for C23H24N8OS as (M+H)⁺ 461.1.

¹H NMR (400 MHz, DMSO) δ 12.29 (s, 1H), 12.19-11.65 (m, 1H), 7.96-7.84 (m, 2H), 7.77 (d, J=7.28 Hz, 2H), 7.54 (d, J=1.25 Hz, 2H), 7.44-7.28 (m, 2H), 7.24-7.10 (m, 1H), 6.84 (s, 1H), 4.90-4.41 (m, 2H), 3.57-3.21 (m, 2H), 3.00 (t, J=10.67 Hz, 1H), 2.31-2.22 (m, 3H), 2.16 (d, J=10.04 Hz, 1H), 2.04-1.80 (m, 2H), 1.76-1.57 (m, 1H).

### Example D-173: Synthesis of 5-[(2R,3R)-3-(4-cyclopropyl-2-fluorobenzamido)-2-methylpiperidin-1-yl]-3-{[3-(morpholin-4-ylmethyl)-1,2-thiazol-5-yl]amino}pyrazine-2-carboxamide (D-173)

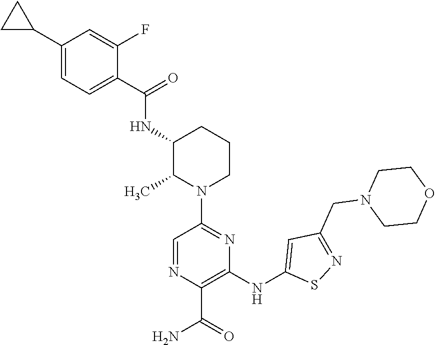 [SMILES: C[C@@H]1[C@H](NC(=O)c2ccc(C3CC3)cc2F)CCCN1c1cnc(C(N)=O)c(Nc2cc(CN3CCOCC3)ns2)n1]

In a similar manner as described in Example 54, 5-[(2R,3R)-3-(4-cyclopropyl-2-fluorobenzamido)-2-methylpiperidin-1-yl]-3-{[3-(morpholin-4-ylmethyl)-1,2-thiazol-5-yl]amino}pyrazine-2-carboxamide was prepared. MS found for C29H35FN8O3S as (M+H)⁺ 595.1.

¹H NMR (400 MHz, DMSO) δ 12.33 (s, 1H), 8.31 (d, J=7.28 Hz, 1H), 7.90 (br. s., 1H), 7.83 (s, 1H), 7.56 (br. s., 1H), 7.46 (t, J=7.84 Hz, 1H), 7.04-6.97 (m, 2H), 6.95 (s, 1H), 5.13 (br. s., 1H), 4.39 (br. s., 1H), 4.14-3.97 (m, 1H), 3.63-3.53 (m, 4H), 3.52-3.41 (m, 2H), 3.20-3.08 (m, 1H), 2.43-2.34 (m, 4H), 2.06-1.96 (m, 1H), 1.95-1.80 (m, 2H), 1.76-1.54 (m, 2H), 1.23 (d, J=6.90 Hz, 3H), 1.07-0.98 (m, 2H), 0.80-0.73 (m, 2H).

### Example D-174: Synthesis of 5-[(3R)-3-(6-cyclopropyl-8-fluoro-1-oxo-1,2-dihydroisoquinolin-2-yl)piperidin-1-yl]-3-{[4-(4-methylpiperazin-1-yl)phenyl]amino}pyrazine-2-carboxamide (D-174)

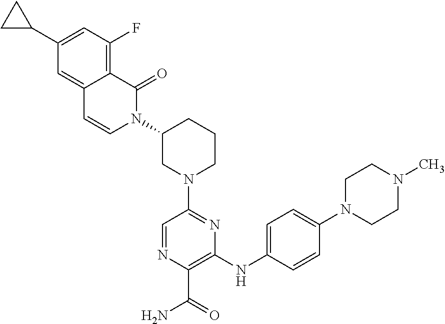 [SMILES: CN1CCN(c2ccc(Nc3nc(N4CCC[C@@H](n5ccc6cc(C7CC7)cc(F)c6c5=O)C4)cnc3C(N)=O)cc2)CC1]

In a similar manner as described in Example D-165, 5-[(3R)-3-(6-cyclopropyl-8-fluoro-1-oxo-1,2-dihydroisoquinolin-2-yl)piperidin-1-yl]-3-{[4-(4-methylpiperazin-1-yl)phenyl]amino}pyrazine-2-carboxamide (D-174) was prepared. MS found for C33H37FN8O2 as (M+H)⁺ 597.6.

¹H NMR (500 MHz, DMSO) δ 10.80 (br. s., 1H), 7.72 (br. s., 1H), 7.69 (s, 1H), 7.59 (d, J=7.34 Hz, 1H), 7.36-7.27 (m, 3H), 7.24 (d, J=1.47 Hz, 1H), 6.98 (d, J=13.21 Hz, 1H), 6.65-6.56 (m, 3H), 4.93-4.82 (m, 1H), 4.54 (d, J=10.76 Hz, 1H), 4.33 (d, J=12.23 Hz, 1H), 3.23-3.02 (m, 2H), 2.84-2.66 (m, 4H), 2.35-2.22 (m, 4H), 2.18 (s, 3H), 2.15-1.61 (m, 5H), 1.14-1.06 (m, 2H), 0.90-0.82 (m, 2H).

### Example D-175: Synthesis of 5-[(2R,3R)-3-[(dimethylcarbamoyl)amino]-2-methylpiperidin-1-yl]-3-[(3-methyl-1,2-thiazol-5-yl)amino]pyridine-2-carboxamide (D-175)

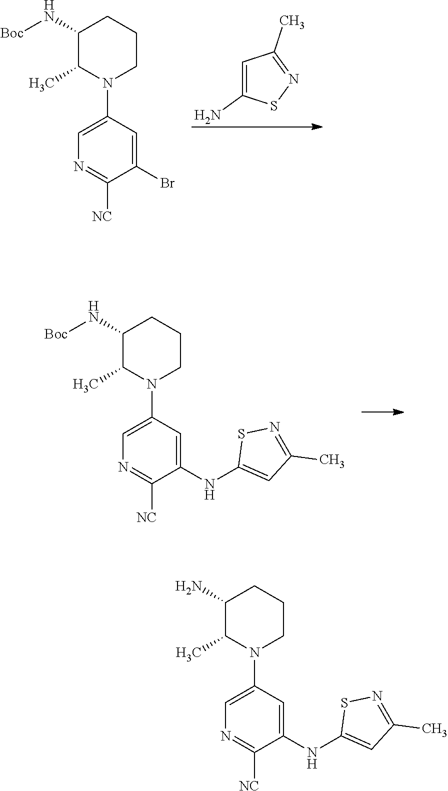

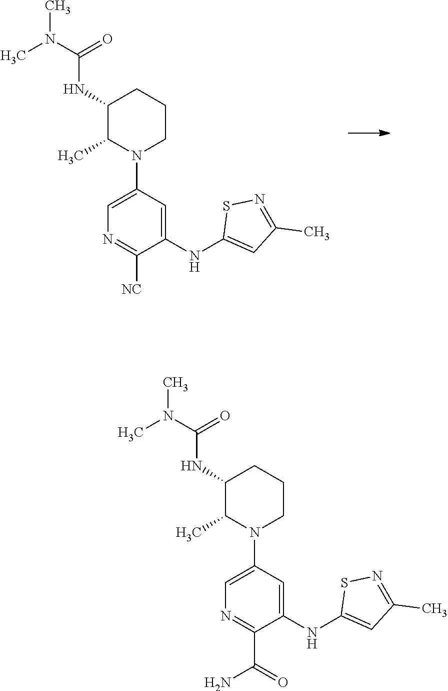

N-[(2R,3R)-1-(5-bromo-6-cyanopyridin-3-yl)-2-methylpiperidin-3-yl]carbamate (150.0 mg, 0.38 mmol), 3-methyl-1,2-thiazol-5-amine (86.0 mg, 0.57 mmol), and Cs₂CO₃ (498.0 mg, 1.52 mmol) were suspended in dioxane (6 mL), Pd(OAc)₂ (20.0 mg, 0.089 mmol) and (+/−) BINAP (48.0 mg, 0.076) were added while degassing with nitrogen. The mixture was heated at 90° C. overnight for 5 h. The solvent was evaporated and the residue purified by silica flash chromatography with 20 to 100% ethyl acetate in cyclohexane to give tert-butyl N-[(2R,3R)-1-{6-cyano-5-[(3-methyl-1,2-thiazol-5-yl)amino]pyridin-3-yl}-2-methylpiperidin-3-yl]carbamate (140.0 mg, 86% yield) as a yellow oil. MS found for C21H28N6O2S as (M+H)⁺ 429.1.

tert-butyl N-[(2R,3R)-1-{6-cyano-5-[(3-methyl-1,2-thiazol-5-yl)amino]pyridin-3-yl}-2-methylpiperidin-3-yl]carbamate (140.0 mg, 0.33 mmol) was dissolved in HCl in MeOH 1.25 N (2.1 mL, 2.64 mmol) and stirred at room temperature for 3 h. The solvent was evaporated to give a product that was passed through an SCX cartridge. Evaporation of the ammonia fractions gave 5-[(2R,3R)-3-amino-2-methylpiperidin-1-yl]-3-[(3-methyl-1,2-thiazol-5-yl)amino]pyridine-2-carbonitrile (108.0 mg, quant. yield). MS found for C16H20N6S as (M+H)⁺ 329.2.

5-[(2R,3R)-3-amino-2-methylpiperidin-1-yl]-3-[(3-methyl-1,2-thiazol-5-yl)amino]pyridine-2-carbonitrile (108.0 0.33 mmol) was dissolved in DMF (2 mL), dimethylcarbamyl chloride (34 μL, 0.36 mmol) was added followed by DIPEA (172.0 μL, 0.99 mmol). The mixture was stirred at room temperature for 1 h. MeOH (10 mL) was added and the mixture evaporated. The residue was purified by silica flash chromatography with ethyl acetate to give 1-[(2R,3R)-1-{6-cyano-5-[(3-methyl-1,2-thiazol-5-yl)amino]pyridin-3-yl}-2-methylpiperidin-3-yl]-3,3-dimethylurea (80.0 mg, 61% yield) as a yellow oil. MS found for C19H25N7OS as (M+H)⁺ 400.0.

1-[(2R,3R)-1-{6-cyano-5-[(3-methyl-1,2-thiazol-5-yl)amino]pyridin-3-yl}-2-methylpiperidin-3-yl]-3,3-dimethylurea (80.0 mg, 0.20 mmol) was dissolved in MeOH/DMSO (3 mL/1 mL), TEA (400.0 μL), NaOH (20.0 mg, 0.48 mmol) and H₂O₂ (80.0 μL) were added. The mixture was stirred overnight at room temperature then diluted with H₂O and extracted with DCM. The combined organic phases were passed through a phase separator concentrated and purified by silica flash chromatography with 0 to 10% MeOH in DCM to give 5-[(2R,3R)-3-[(dimethylcarbamoyl)amino]-2-methylpiperidin-1-yl]-3-[(3-methyl-1,2-thiazol-5-yl)amino]pyridine-2-carboxamide (D-175) (20.0 mg, 24% yield) as a pale brown solid. MS found for C19H27N7O2S as (M+H)⁺ 418.1.

¹H NMR (400 MHz, DMSO) δ 12.12-11.90 (m, 1H), 8.01 (d, J=2.41 Hz, 1H), 7.87 (d, J=2.41 Hz, 1H), 7.55 (d, J=1.97 Hz, 1H), 6.89 (d, J=2.19 Hz, 1H), 6.85 (s, 1H), 6.09 (d, J=6.80 Hz, 1H), 4.43-4.29 (m, 1H), 3.81-3.60 (m, 2H), 3.11-2.99 (m, 1H), 2.81 (s, 6H), 2.32 (s, 3H), 1.79 (d, J=12.50 Hz, 2H), 1.58 (d, J=7.67 Hz, 2H), 1.05 (d, J=6.80 Hz, 3H).

### Example D-176: Synthesis of 5-[(2R,3R)-3-(4-cyclopropyl-2-fluorobenzamido)-2-methylpiperidin-1-yl]-3-{[3-(piperidin-1-ylmethyl)-1,2-thiazol-5-yl]amino}pyrazine-2-carboxamide (D-176)

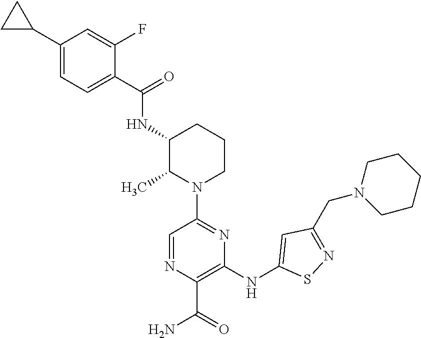 [SMILES: C[C@@H]1[C@H](NC(=O)c2ccc(C3CC3)cc2F)CCCN1c1cnc(C(N)=O)c(Nc2cc(CN3CCCCC3)ns2)n1]

In a similar manner as described in Example 54, 5-[(2R,3R)-3-(4-cyclopropyl-2-fluorobenzamido)-2-methylpiperidin-1-yl]-3-{[3-(piperidin-1-ylmethyl)-1,2-thiazol-5-yl]amino}pyrazine-2-carboxamide was prepared. MS found for C30H37FN8O2S as (M+H)⁺ 593.1.

¹H NMR (500 MHz, DMSO) δ 12.28 (s, 1H), 8.32 (d, J=7.41 Hz, 1H), 7.91 (s, 1H), 7.82 (s, 1H), 7.57 (s, 1H), 7.46 (t, J=7.82 Hz, 1H), 7.06-6.97 (m, 2H), 6.91 (s, 1H), 5.38-4.96 (m, 1H), 4.55-4.25 (m, 1H), 4.13-3.97 (m, 1H), 3.46-3.36 (m, 2H), 3.15 (t, J=12.21 Hz, 1H), 2.43-2.24 (m, 4H), 2.05-1.96 (m, 1H), 1.94-1.58 (m, 4H), 1.49 (quin, J=5.56 Hz, 4H), 1.37 (d, J=4.12 Hz, 2H), 1.23 (d, J=6.86 Hz, 3H), 1.03 (dd, J=8.37, 2.33 Hz, 2H), 0.79-0.72 (m, 2H).

### Example D-177: Synthesis of 3-{[4-(1-cyclopentyl-4-methylpiperidin-4-yl)phenyl]amino}-5-[(3R)-3-(6-cyclopropyl-1-oxo-1,2-dihydroisoquinolin-2-yl)piperidin-1-yl]pyrazine-2-carboxamide (D-177)

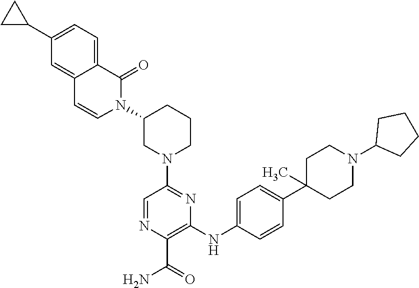 [SMILES: CC1(c2ccc(Nc3nc(N4CCC[C@@H](n5ccc6cc(C7CC7)ccc6c5=O)C4)cnc3C(N)=O)cc2)CCN(C2CCCC2)CC1]

In a similar manner as described in Example D-165, 3-{[4-(1-cyclopentyl-4-methylpiperidin-4-yl)phenyl]amino}-5-[(3R)-3-(6-cyclopropyl-1-oxo-1,2-dihydroisoquinolin-2-yl)piperidin-1-yl]pyrazine-2-carboxamide (D-177) was prepared. MS found for C39H47N7O2 as (M+H)⁺ 646.2.

¹H NMR (400 MHz, DMSO) δ 11.06-10.93 (m, 1H), 8.17 (d, J=8.33 Hz, 1H), 7.78-7.66 (m, 2H), 7.54 (d, J=7.67 Hz, 1H), 7.39 (d, J=8.55 Hz, 2H), 7.36-7.32 (m, 1H), 7.32-7.28 (m, 1H), 7.20 (dd, J=8.33, 1.53 Hz, 1H), 6.91 (d, J=7.67 Hz, 2H), 6.59 (d, J=7.67 Hz, 1H), 4.91 (t, J=11.51 Hz, 1H), 4.56 (d, J=12.28 Hz, 1H), 4.32 (d, J=12.28 Hz, 1H), 3.19 (t, J=1.84 Hz, 1H), 3.10 (t, J=12.39 Hz, 1H), 2.33-1.86 (m, 9H), 1.78-1.12 (m, 13H), 1.05 (dd, J=6.36, 2.19 Hz, 2H), 0.86 (s, 3H), 0.82-0.75 (m, 2H).

### Example D-178: Synthesis of 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyridine-2-carboxamide (D-178)

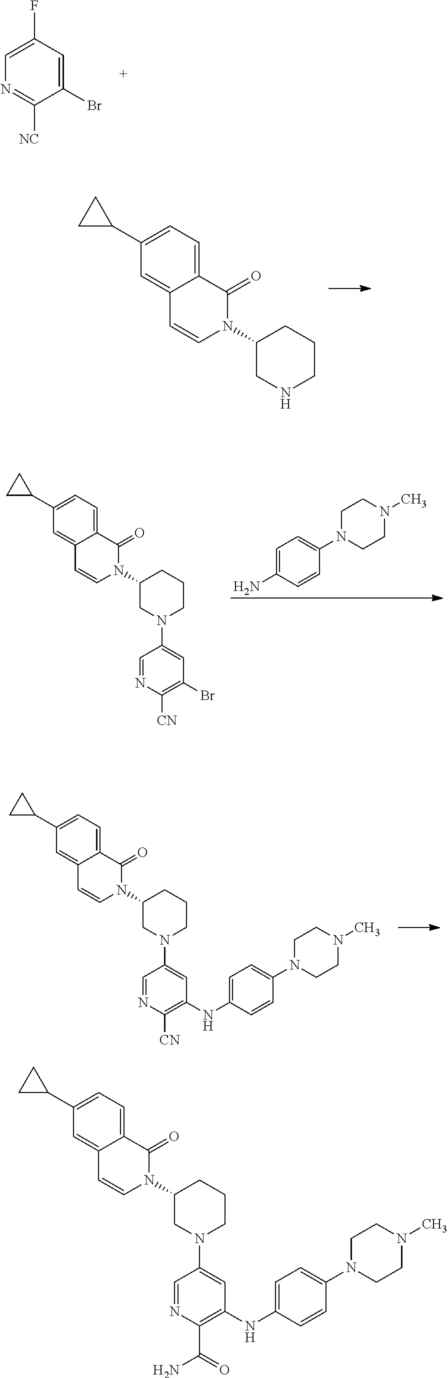 [SMILES: N#Cc1ncc(F)cc1Br ; O=c1c2ccc(C3CC3)cc2ccn1[C@@H]1CCCNC1 ; N#Cc1ncc(N2CCC[C@@H](n3ccc4cc(C5CC5)ccc4c3=O)C2)cc1Br ; CN1CCN(c2ccc(N)cc2)CC1 ; CN1CCN(c2ccc(Nc3cc(N4CCC[C@@H](n5ccc6cc(C7CC7)ccc6c5=O)C4)cnc3C#N)cc2)CC1 ; CN1CCN(c2ccc(Nc3cc(N4CCC[C@@H](n5ccc6cc(C7CC7)ccc6c5=O)C4)cnc3C(N)=O)cc2)CC1]

6-Cyclopropyl-2-[(3R)-piperidin-3-yl]-1,2-dihydroisoquinolin-1-one was prepared as in Example D-165.

To a suspension of 6-cyclopropyl-2-[(3R)-piperidin-3-yl]-1,2-dihydroisoquinolin-1-one (100.0 mg, 0.33 mmol) in DMF (2 mL) DIPEA (0.2 mL, 0.99 mmol) and 3-bromo-5-fluoropyridine-2-carbonitrile (75 mg, 0.36 mmol) were added. The mixture was stirred at 90° C. for 3 h, and then it was purified by silica NH flash chromatography with 20 to 100% ethyl acetate in cyclohexane to give 3-bromo-5-[(3R)-3-(6-cyclopropyl-1-oxo-1,2-dihydroisoquinolin-2-yl)piperidin-1-yl]pyridine-2-carbonitrile (126.0 mg, 85% yield) as a white solid. MS found for C23H21BrN4O as (M+H)⁺ 449.1, 451.1.

To a solution of 3-bromo-5-[(3R)-3-(6-cyclopropyl-1-oxo-1,2-dihydroisoquinolin-2-yl)piperidin-1-yl]pyridine-2-carbonitrile (126.0 mg, 0.28 mmol) in dioxane (3 ml), Cs₂CO₃ (365.0 mg, 01.12 mmol), 4-(4-methylpiperazin-1-yl)aniline (107.0 mg, 0.56 mmol), (+/−) BINAP (70.0 mg, 0.112 mmol) and Pd(OAc)₂ (25.0 mg, 0.112 mmol) were added and the mixture stirred for 3 h at 100° C., then it was left to cool to room temperature. The mixture purified by silica NH flash chromatography with 50 to 100% ethyl acetate in cyclohexane to give 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyridine-2-carbonitrile (63.0 mg, 40% yield) as a yellow solid. MS found for C34H37N7O as (M+H)⁺ 560.2.

To a solution of 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyridine-2-carbonitrile (63.0 mg, 0.11 mmol) in MeOH/DMSO (2 mL/0.2 mL), TEA (0.5 mL), NaOH (13.0 mg, 0.32 mmol) and H₂O₂ (50 μL) were added.

The mixture was stirred overnight at room temperature, and then it was partitioned between DCM and H₂O. The combined organic phases were dried over Na₂SO₄ and concentrated. The crude was purified by silica NH flash chromatography with 50 to 100 ethyl acetate in cyclohexane to give 5-[(3R)-3-(6-cyclopropyl-1-oxo-1,2-dihydroisoquinolin-2-yl)piperidin-1-yl]-3-{[4-(4-methylpiperazin-1-yl)phenyl]amino}pyridine-2-carboxamide (D-178) (51.3 mg, 81% yield) as a yellow solid. MS found for C₃₄H₃₉N₇O₂ as (M+H)⁺ 578.2.

¹H NMR (500 MHz, DMSO) δ 10.19 (s, 1H), 8.18 (d, J=8.23 Hz, 1H), 7.85-7.78 (m, 1H), 7.76 (d, J=2.47 Hz, 1H), 7.53 (d, J=7.68 Hz, 1H), 7.36 (d, J=1.65 Hz, 1H), 7.25 (d, J=2.74 Hz, 1H), 7.23 (dd, J=8.51, 1.65 Hz, 1H), 7.12 (d, J=8.78 Hz, 2H), 6.83 (d, J=9.06 Hz, 2H), 6.74 (d, J=2.47 Hz, 1H), 6.59 (d, J=7.41 Hz, 1H), 4.93 (tt, J=11.63, 3.74 Hz, 1H), 3.89 (d, J=14.00 Hz, 1H), 3.69 (d, J=12.90 Hz, 1H), 3.26 (m, J=11.80 Hz, 1H), 3.04-2.86 (m, 5H), 2.36 (td, J=7.14, 3.84 Hz, 4H), 2.20 (s, 3H), 2.12-2.02 (m, 2H), 1.94-1.62 (m, 3H), 1.11-1.03 (m, 2H), 0.86-0.77 (m, 2H).

### Example D-179: Synthesis of 5-[(3R)-3-(6-cyclopropyl-1-oxo-1,2-dihydroisoquinolin-2-yl)piperidin-1-yl]-3-[5-(4-methylpiperazin-1-yl)pyridin-2-yl]amino) pyrazine-2-carboxamide (D-179)

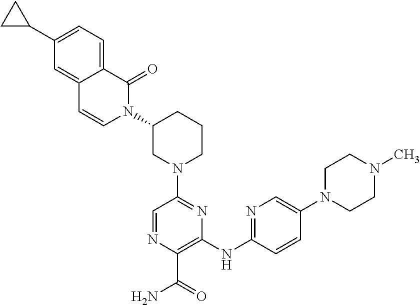 [SMILES: CN1CCN(c2ccc(Nc3nc(N4CCC[C@@H](n5ccc6cc(C7CC7)ccc6c5=O)C4)cnc3C(N)=O)nc2)CC1]

In a similar manner as described in Example D-165, 5-[(3R)-3-(6-cyclopropyl-1-oxo-1,2-dihydroisoquinolin-2-yl)piperidin-1-yl]-3-{[5-(4-methylpiperazin-1-yl)pyridin-2-yl]amino}pyrazine-2-carboxamide (D-179) was prepared. MS found for C32H37N9O2 as (M+H)⁺ 580.2.

¹H NMR (500 MHz, DMSO) δ 11.38-11.17 (m, 1H), 8.20 (d, J=8.23 Hz, 1H), 7.99-7.92 (m, 1H), 7.92-7.88 (m, 1H), 7.84-7.75 (m, 2H), 7.59 (d, J=7.68 Hz, 1H), 7.40 (d, J=1.37 Hz, 1H), 7.39-7.37 (m, 1H), 7.24 (dd, J=8.51, 1.65 Hz, 1H), 6.95-6.73 (m, 1H), 6.67-6.61 (m, 1H), 5.05-4.87 (m, 1H), 4.56 (d, J=9.88 Hz, 1H), 4.39 (d, J=11.25 Hz, 1H), 3.25 (t, J=11.53 Hz, 1H), 3.15 (t, J=12.76 Hz, 1H), 2.82 (br. s., 4H), 2.43-2.30 (m, 4H), 2.20 (s, 3H), 2.19-2.13 (m, 1H), 2.12-2.05 (m, 1H), 2.03-1.67 (m, 3H), 1.13-1.06 (m, 2H), 0.88-0.77 (m, 2H).

### Example D-180: Synthesis of 5-[(2R,3R)-3-[(dimethylcarbamoyl)amino]-2-methylpiperidin-1-yl]-3-{[5-(4-methylpiperazin-1-yl)pyridin-2-yl]amino}pyrazine-2-carboxamide (D-180)

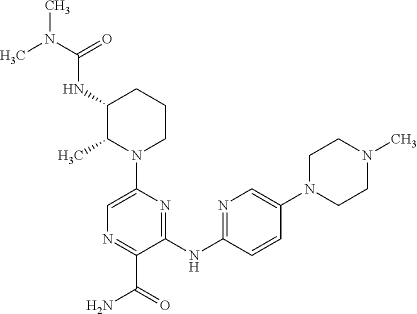

In a similar manner as described in Example 59, 5-[(2R,3R)-3-[(dimethylcarbamoyl)amino]-2-methylpiperidin-1-yl]-3-{[5-(4-methylpiperazin-1-yl)pyridin-2-yl]amino}pyrazine-2-carboxamide (D-180) was prepared using 1-[(2R,3R)-1-(6-chloro-5-cyanopyrazin-2-yl)-2-methylpiperidin-3-yl]-3,3-dimethylurea. MS found for C24H36N10O2 as (M+H)⁺ 497.4.

¹H NMR (500 MHz, DMSO) δ 11.44 (s, 1H), 8.16 (d, J=8.78 Hz, 1H), 7.98 (d, J=3.02 Hz, 1H), 7.74 (br. s., 1H), 7.67-7.61 (m, 1H), 7.41-7.30 (m, 2H), 6.11 (d, J=6.86 Hz, 1H), 5.05 (br. s., 1H), 4.24-4.05 (m, 1H), 3.80-3.60 (m, 1H), 3.12-3.06 (m, 4H), 3.07-2.96 (m, 1H), 2.85 (s, 6H), 2.47-2.41 (m, 4H), 2.22 (s, 3H), 1.88-1.46 (m, 4H), 1.05 (d, J=6.86 Hz, 3H).

### Example D-181: Synthesis of 5-[(2S,5R)-5-(4-cyclopropyl-2-fluorobenzamido)-2-methylpiperidin-1-yl]-3-[(3-methyl-1,2-thiazol-5-yl)amino]pyrazine-2-carboxamide (D-181)

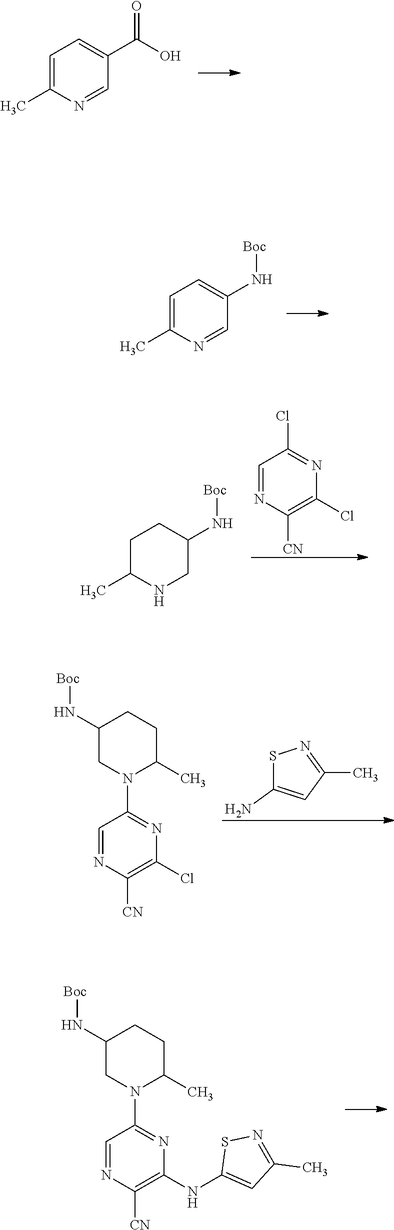

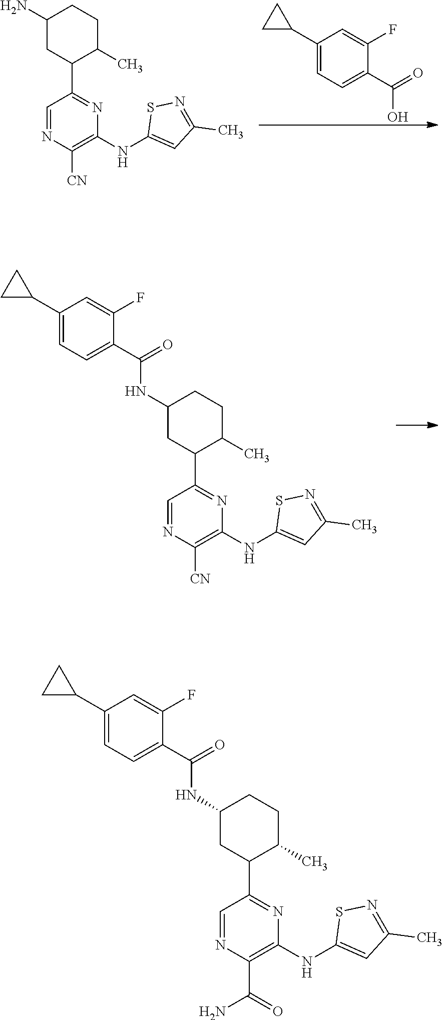 [SMILES: Cc1cc(Nc2nc(C3CC(N)CCC3C)cnc2C#N)sn1 ; O=C(O)c1ccc(C2CC2)cc1F ; Cc1cc(Nc2nc(C3CC(NC(=O)c4ccc(C5CC5)cc4F)CCC3C)cnc2C#N)sn1 ; Cc1cc(Nc2nc(C3C[C@H](NC(=O)c4ccc(C5CC5)cc4F)CC[C@@H]3C)cnc2C(N)=O)sn1]

6-Methylpyridine-3-carboxylic acid (2.0 g, 14.6 mmol) and TEA (4 mL) were dissolved in t-BuOH (50 mL) and stirred at room temperature for 10 min, DPPA (3.150 ml, 14.6 mmol) was added and the reaction refluxed for 5 h. The reaction was let to cool to room temperature then concentrated. The crude material was purified by silica NH flash chromatography with 20 to 50% ethyl acetate in cyclohexane to give tert-butyl N-(6-methylpyridin-3-yl)carbamate (1.67 g, 55% yield) as a white solid. MS found for C11H16N2O2 as (M+H)⁺ 209.1.

Tert-butyl N-(6-methylpyridin-3-yl)carbamate (1.67 g, 8.02 mmol) was dissolved in AcOH (100 mL). PtO₂ (500 mg) was added and the mixture was stirred overnight under H₂ atmosphere (1 atm). The catalyst was filtered off, the solvent evaporated, the residue taken up with DCM and washed with NaHCO₃ sat. aqueous solution. The combined organic phases were concentrated under reduced pressure to give tert-butyl N-(6-methylpiperidin-3-yl)carbamate (1.4 g, 81% yield) as diastereoisomeric mixture. MS found for C11H16N2O2 as (M+H)⁺ 215.4.

3,5-Dichloropyrazine-2-carbonitrile (1.25 g, 7.2 mmol), tert-butyl N-(6-methylpiperidin-3-yl)carbamate (1.45 g, 6.5 mmol) and DIPEA (2.3 mL, 13.1 mmol) were dissolved in DMF (40 mL) and stirred at room temperature for 4 h. The reaction was concentrated and purified by silica flash chromatography with 20 to 50% ethyl acetate in cyclohexane to give tert-butyl N-[1-(6-chloro-5-cyanopyrazin-2-yl)-6-methylpiperidin-3-yl]carbamate (2.1 g, 83% yield). MS found for C16H22ClN5O2 as (M+H)⁺ 353.0.

Tert-butyl N-[1-(6-chloro-5-cyanopyrazin-2-yl)-6-methylpiperidin-3-yl]carbamate (1.0 g, 2.84 mmol), 5-amino-3-methyl-isothiazole (815.0 mg, 4.26 mmol) and Cs₂CO₃ (3.70 g, 11.4 mmol) were dissolved in dioxane (30 mL). The mixture was degassed with nitrogen and (+/−) BINAP (355.0 mg, 0.57 mmol) and Pd(OAc)₂ (127.9 mg, 0.57 mmol) were added. The mixture was stirred at 90° C. overnight for 2 h. The solvent was removed by evaporation and the residue purified by silica flash chromatography with 0 to 100% ethyl acetate in cyclohexane to give tert-butyl N-(1-{5-cyano-6-[(3-methyl-1,2-thiazol-5-yl)amino]pyrazin-2-yl}-6-methylpiperidin-3-yl)carbamate (1.2 g, 98% yield). MS found for C20H27N7O2S as (M+H)⁺ 430.5.

Tert-butyl N-(1-{5-cyano-6-[(3-methyl-1,2-thiazol-5-yl)amino]pyrazin-2-yl}-6-methylpiperidin-3-yl)carbamate was dissolved in TFA (4 mL) and H₂SO₄ (400 μL). The reaction was stirrer at room temperature for 3 h. The reaction was concentrated under reduced pressure. The residue was dissolved in DCM and washed with NaHCO₃ sat. aqueous solution. The DCM phase was dried over Na₂SO₄, concentrated and purified through SCX cartridge eluting with NH₃ 7 N in MeOH. The solution was concentrated in vacuo to give 5-(5-amino-2-methylpiperidin-1-yl)-3-[(3-methyl-1,2-thiazol-5-yl)amino]pyrazine-2-carbonitrile (560.0 mg, 60% yield). MS found for C15H9N7S as (M+H)⁺ 330.3.

5-(5-amino-2-methylpiperidin-1-yl)-3-[(3-methyl-1,2-thiazol-5-yl)amino]pyrazine-2-carbonitrile (560.0 mg, 1.7 mmol), 4-cyclopropyl-2-fluorobenzoic acid (398.0 mg, 2.21 mmol) and PyBop (1150.1 mg, 2.21 mmol) were dissolved in DMF (15 mL), DIPEA (1.48 mL, 8.5 mmol) was added. The mixture was stirred for 4 h. DMF was evaporated and the residue purified by silica flash chromatography with 0 to 10% MeOH in DCM to give N-(1-{5-cyano-6-[(3-methyl-1,2-thiazol-5-yl)amino]pyrazin-2-yl}-6-methylpiperidin-3-yl)-4-cyclopropyl-2-fluorobenzamide (430.0 mg, 51% yield). MS found for C25H26FN7OS as (M+H)⁺ 492.5.

N-(1-{5-cyano-6-[(3-methyl-1,2-thiazol-5-yl)amino]pyrazin-2-yl}-6-methylpiperidin-3-yl)-4-cyclopropyl-2-fluorobenzamide (430.0 mg, 0.87 mmol) was dissolved in MeOH/DMSO (12 mL/4 mL), NaOH (85.0 mg, 2.09 mmol) and H₂O₂ (240.0 μL) were added. The mixture was stirred at room temperature for 4 h, then H₂O₂ (240.0 μL) was added and the reaction stirred at 50° C. for 6 h. The reaction was concentrated, treated with NaHCO₃ sat. aqueous solution and extracted with DCM. The combined organic phases concentrated and purified by preparative HPLC then by chiral HPLC to give 5-[(2S,5R)-5-(4-cyclopropyl-2-fluorobenzamido)-2-methylpiperidin-1-yl]-3-[(3-methyl-1,2-thiazol-5-yl)amino]pyrazine-2-carboxamide (D-181) (75.5 mg, 17% yield) as a yellow solid. MS found for C25H28FN7O2S as (M+H)⁺ 510.1.

¹H NMR (400 MHz, CDCl₃) δ 11.90 (br. s., 1H), 8.01 (t, J=8.22 Hz, 1H), 7.71 (s, 1H), 7.52-7.38 (m, 1H), 6.99 (dd, J=8.22, 1.17 Hz, 1H), 6.81 (dd, J=13.69, 1.17 Hz, 1H), 6.68 (dd, J=14.09, 7.04 Hz, 1H), 6.63 (s, 1H), 5.37 (br. s., 1H), 5.29-5.18 (m, 1H), 4.57 (dd, J=12.91, 4.30 Hz, 1H), 4.20-4.06 (m, 1H), 3.02 (dd, J=12.91, 11.35 Hz, 1H), 2.43 (s, 3H), 2.11-1.76 (m, 5H), 1.38 (d, J=7.04 Hz, 3H), 1.14-1.05 (m, 2H), 0.81-0.75 (m, 2H).

### Example D-182: Synthesis of 3-{[4-(4-cyclopentylpiperazin-1-yl)phenyl]amino}-5-[(2R,3R)-3-[(dimethylcarbamoyl)amino]-2-methylpiperidin-1-yl]pyrazine-2-carboxamide (D-182)

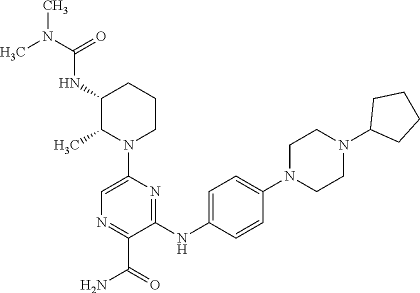 [SMILES: C[C@@H]1[C@H](NC(=O)N(C)C)CCCN1c1cnc(C(N)=O)c(Nc2ccc(N3CCN(C4CCCC4)CC3)cc2)n1]

In a similar manner as described in Example 59, 3-{[4-(4-cyclopentylpiperazin-1-yl)phenyl]amino}-5-[(2R,3R)-3-[(dimethylcarbamoyl)amino]-2-methylpiperidin-1-yl]pyrazine-2-carboxamide (D-182) was prepared using 1-[(2R,3R)-1-(6-chloro-5-cyanopyrazin-2-yl)-2-methylpiperidin-3-yl]-3,3-dimethylurea. MS found for C29H43N9O2 as (M+H)⁺ 550.3.

¹H NMR (500 MHz, DMSO) δ 11.04 (s, 1H), 7.70 (br. s., 1H), 7.59-7.51 (m, 1H), 7.49-7.42 (m, 2H), 7.26 (br. s., 1H), 6.88 (d, J=9.06 Hz, 2H), 6.09 (d, J=6.86 Hz, 1H), 5.01-4.77 (m, 1H), 4.14 (br. s., 1H), 3.78-3.59 (m, 1H), 3.12-3.02 (m, 4H), 3.01-2.93 (m, 1H), 2.84 (s, 6H), 2.57-2.52 (m, 4H), 2.49-2.44 (m, 1H), 1.87-1.28 (m, 12H), 1.04 (d, J=6.86 Hz, 3H).

### Example D-183: Synthesis of 3-{[4-(1-cyclopentyl-4-methylpiperidin-4-yl)phenyl]amino}-5-[(2R,3R)-3-[4-(dimethylamino)benzamido]-2-methylpiperidin-1-yl]pyrazine-2-carboxamide (D-183)

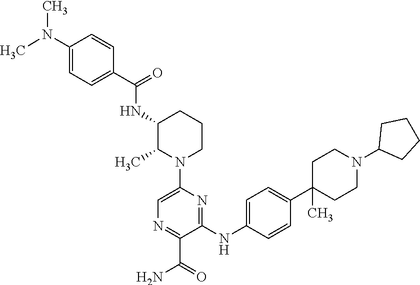 [SMILES: C[C@@H]1[C@H](NC(=O)c2ccc(N(C)C)cc2)CCCN1c1cnc(C(N)=O)c(Nc2ccc(C3(C)CCN(C4CCCC4)CC3)cc2)n1]

In a similar manner as described in Example 7, 3-{[4-(1-cyclopentyl-4-methylpiperidin-4-yl)phenyl]amino}-5-[(2R,3R)-3-[4-(dimethylamino)benzamido]-2-methylpiperidin-1-yl]pyrazine-2-carboxamide (D-183) was prepared using 4-(1-cyclopentyl-4-methylpiperidin-4-yl)aniline. 4-(1-Cyclopentyl-4-methylpiperidin-4-yl)aniline was prepared as reported in WO 2015/084998 A1. MS found for C37H50N8O2 as (M+H)⁺ 639.3.

¹H NMR (500 MHz, DMSO) δ 11.17 (br. s., 1H), 8.04 (d, J=7.41 Hz, 1H), 7.84 (d, J=8.78 Hz, 2H), 7.74 (br. s., 1H), 7.63 (s, 1H), 7.54 (d, J=8.64 Hz, 2H), 7.31 (br. s., 1H), 7.20 (d, J=8.37 Hz, 2H), 6.71 (d, J=8.92 Hz, 2H), 5.20 (br. s., 1H), 4.26-3.99 (m, 2H), 3.12-3.03 (m, 1H), 2.97 (s, 6H), 2.47-2.16 (m, 5H), 2.06-1.16 (m, 16H), 1.12-0.99 (m, 6H).

### Example D-184: Synthesis of 5-[(3R)-3-(6-cyclopropyl-1-oxo-1,2-dihydroisoquinolin-2-yl)piperidin-1-yl]-3-[(1-methyl-1H-pyrazol-4-yl)amino]pyrazine-2-carboxamide (D-184)

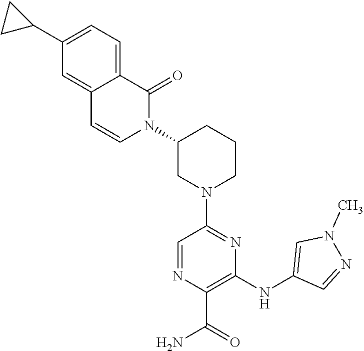 [SMILES: Cn1cc(Nc2nc(N3CCC[C@@H](n4ccc5cc(C6CC6)ccc5c4=O)C3)cnc2C(N)=O)cn1]

In a similar manner as described in Example D-165, 5-[(3R)-3-(6-cyclopropyl-1-oxo-1,2-dihydroisoquinolin-2-yl)piperidin-1-yl]-3-[(1-methyl-1H-pyrazol-4-yl)amino]pyrazine-2-carboxamide (D-184) was prepared. MS found for C26H28N8O2 as (M+H)⁺ 485.2.

¹H NMR (400 MHz, DMSO) δ 10.83 (s, 1H), 8.14 (d, J=8.44 Hz, 1H), 7.86 (s, 1H), 7.72 (br. s., 1H), 7.67 (s, 1H), 7.61 (d, J=7.56 Hz, 1H), 7.47 (s, 1H), 7.36 (d, J=1.50 Hz, 1H), 7.30 (br. s., 1H), 7.23 (dd, J=8.44, 1.53 Hz, 1H), 6.63 (d, J=7.56 Hz, 1H), 4.98-4.84 (m, 1H), 4.56 (d, J=10.96 Hz, 1H), 4.41 (d, J=13.26 Hz, 1H), 3.58 (s, 3H), 3.38-3.22 (m, 1H), 3.10 (t, J=13.04 Hz, 1H), 2.27-2.12 (m, 1H), 2.11-2.02 (m, 1H), 2.01-1.88 (m, 2H), 1.81-1.60 (m, 1H), 1.12-0.99 (m, 2H), 0.88-0.75 (m, 2H).

### Example D-185: Synthesis of 5-[(2R,3R)-3-(4-cyclopropylbenzamido)-2-methylpiperidin-1-yl]-3-{[1-(1-methylpiperidin-4-yl)-1H-pyrazol-4-yl]amino}pyrazine-2-carboxamide (D-185)

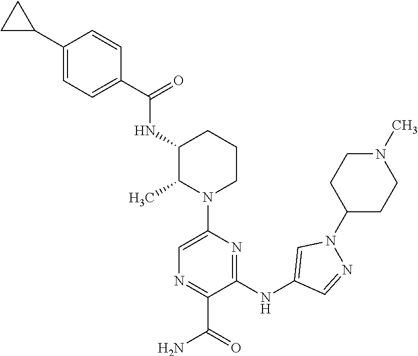 [SMILES: C[C@@H]1[C@H](NC(=O)c2ccc(C3CC3)cc2)CCCN1c1cnc(C(N)=O)c(Nc2cnn(C3CCN(C)CC3)c2)n1]

In a similar manner as described in Example D-269, 5-[(2R,3R)-3-(4-cyclopropylbenzamido)-2-methylpiperidin-1-yl]-3-{[1-(1-methylpiperidin-4-yl)-1H-pyrazol-4-yl]amino}pyrazine-2-carboxamide (D-185) was prepared. MS found for C30H39N9O2 as (M+H)⁺ 558.5.

¹H NMR (500 MHz, DMSO) δ 10.86 (s, 1H), 8.36 (d, J=6.86 Hz, 1H), 8.00 (s, 1H), 7.82 (d, J=8.37 Hz, 2H), 7.68 (br. s., 1H), 7.59 (s, 1H), 7.45 (s, 1H), 7.27 (d, J=1.78 Hz, 1H), 7.18 (d, J=8.23 Hz, 2H), 5.22 (br. s., 1H), 4.23-3.97 (m, 2H), 3.88 (br. s., 1H), 3.17-3.03 (m, 1H), 2.06-1.90 (m, 5H), 1.89-1.54 (m, 7H), 2.67-1.22 (m, 4H), 1.14 (d, J=7.00 Hz, 3H), 1.06-0.96 (m, 2H), 0.78-0.68 (m, 2H).

### Example D-186: Synthesis of 5-[(2R,3R)-3-[5-(4-cyclopropylphenyl)-1H-imidazol-2-yl]-2-methylpiperidin-1-yl]-3-[(3-methyl-1,2-thiazol-5-yl)amino]pyrazine-2-carboxamide (D-186)

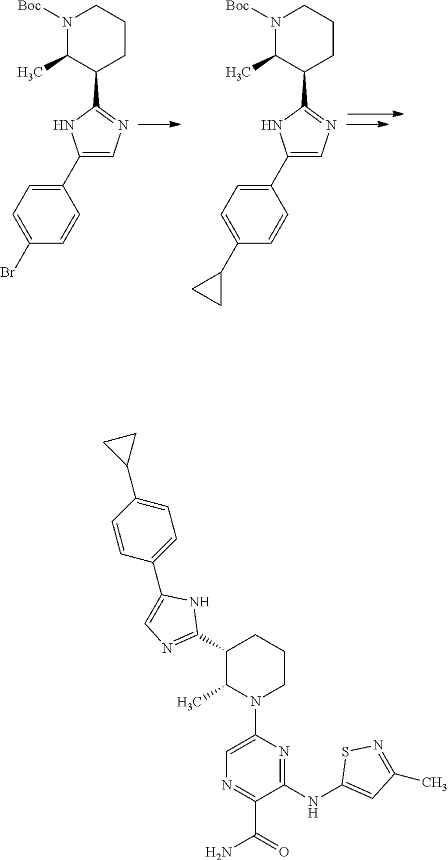 [SMILES: C[C@@H]1[C@H](c2ncc(-c3ccc(Br)cc3)[nH]2)CCCN1C(=O)OC(C)(C)C ; C[C@@H]1[C@H](c2ncc(-c3ccc(C4CC4)cc3)[nH]2)CCCN1C(=O)OC(C)(C)C ; Cc1cc(Nc2nc(N3CCC[C@@H](c4ncc(-c5ccc(C6CC6)cc5)[nH]4)[C@H]3C)cnc2C(N)=O)sn1]

To a solution of tert-butyl (2R,3R)-3-[5-(4-bromophenyl)-1H-imidazol-2-yl]-2-methylpiperidine-1-carboxylate (186.0 mg, 0.44 mmol) in toluene/H₂O (2 ml/0.1 ml), K₃PO₄ (327.0 mg, 1.54 mmol), cyclopropylboronic acid (46.0, 0.53 mmol), PCy₃ (25.0 mg, 0.088 mmol) and Pd(OAc)₂ (10 mg, 0.044 mmol) were added. The mixture was stirred at 100° C. for 1 day, then left to reach room temperature, concentrated and taken up with dichloromethane. The organic phase was separated to give a crude that was purified by NH silica flash chromatography with 10 to 30% ethyl acetate in cyclohexane to give tert-butyl (2R,3R)-3-[5-(4-cyclopropylphenyl)-1H-imidazol-2-yl]-2-methylpiperidine-1-carboxylate (102.0 mg, 36% yield) as a colourless viscous oil. MS found for C23H31N3O2 as (M+H)⁺ 382.18.

In a similar manner as described in Example (D-131) 5-[(2R,3R)-3-[5-(4-cyclopropylphenyl)-1H-imidazol-2-yl]-2-methylpiperidin-1-yl]-3-[(3-methyl-1,2-thiazol-5-yl)amino]pyrazine-2-carboxamide (D-186) was prepared using 3-methyl-1,2-thiazol-5-amine hydrochloride (9.0 mg, 26% yield) as a white solid. MS found for C27H30N8OS as (M+H)⁺ 515.15.

¹H NMR (500 MHz, DMSO) δ 12.30 (s, 1H), 12.10-11.76 (m, 1H), 8.02-7.82 (m, 2H), 7.78-7.42 (m, 3H), 7.53-7.14 (m, 1H), 7.14-6.97 (m, 2H), 6.85 (s, 1H), 5.87-3.90 (m, 2H), 2.30 (s, 3H), 2.25-2.11 (m, 1H), 2.07-1.85 (m, 3H), 1.75-1.60 (m, 1H), 1.05 (d, J=6.85 Hz, 3H), 0.98-0.89 (m, 2H), 0.74-0.60 (m, 2H).

### Example D-187: Synthesis of 5-[(2R,3R)-3-(4-cyclopropylbenzamido)-2-methylpiperidin-1-yl]-3-{(1-[2-(dimethylamino)ethyl]-1H-pyrazol-4-yl}amino)pyrazine-2-carboxamide (D-187)

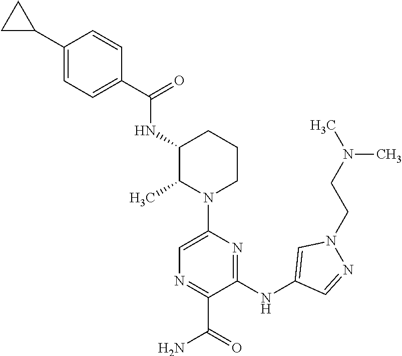 [SMILES: C[C@@H]1[C@H](NC(=O)c2ccc(C3CC3)cc2)CCCN1c1cnc(C(N)=O)c(Nc2cnn(CCN(C)C)c2)n1]

In a similar manner as described in Example D-269, 5-[(2R,3R)-3-(4-cyclopropylbenzamido)-2-methylpiperidin-1-yl]-3-({1-[2-(dimethylamino)ethyl]-1H-pyrazol-4-yl}amino)pyrazine-2-carboxamide (D-187) was prepared. MS found for C28H37N9O2 as (M+H)⁺ 532.6.

¹H NMR (400 MHz, DMSO) δ ppm 10.89 (s, 1H), 8.34 (d, J=6.80 Hz, 1H), 8.04 (s, 1H), 7.82 (d, J=8.33 Hz, 2H), 7.69 (br. s., 1H), 7.57 (s, 1H), 7.45 (s, 1H), 7.27 (br. s., 1H), 7.17 (d, J=8.33 Hz, 2H), 5.45-5.05 (m, 1H), 4.26-3.96 (m, 4H), 3.08 (t, J=12.06 Hz, 1H), 2.46 (d, J=5.92 Hz, 2H), 2.09-1.47 (m, 11H), 1.10 (d, J=6.80 Hz, 3H), 1.05-0.97 (m, 2H), 0.78-0.68 (m, 2H).

### Example D-188: Synthesis of 5-[(2R,3R)-3-(5-cyclopropylpyridine-2-amido)-2-methylpiperidin-1-yl]-3-{[4-(4-methylpiperazin-1-yl)phenyl]amino}pyrazine-2-carboxamide (D-188)

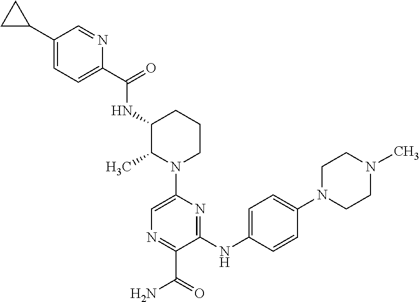 [SMILES: C[C@@H]1[C@H](NC(=O)c2ccc(C3CC3)cn2)CCCN1c1cnc(C(N)=O)c(Nc2ccc(N3CCN(C)CC3)cc2)n1]

In a similar manner as described in Example D-216, 5-[(2R,3R)-3-(5-cyclopropylpyridine-2-amido)-2-methylpiperidin-1-yl]-3-{[4-(4-methylpiperazin-1-yl)phenyl]amino}pyrazine-2-carboxamide (D-188) was prepared. MS found for C31H39N9O2 as (M+H)⁺ 570.6.

¹H NMR (500 MHz, DMSO) δ 10.94 (s, 1H), 8.54 (d, J=8.32 Hz, 1H), 8.50 (d, J=1.96 Hz, 1H), 7.99 (d, J=7.83 Hz, 1H), 7.71 (d, J=1.47 Hz, 1H), 7.64 (dd, J=8.07, 2.20 Hz, 1H), 7.60 (s, 1H), 7.43 (d, J=8.80 Hz, 2H), 7.28 (d, J=1.47 Hz, 1H), 6.79 (d, J=8.80 Hz, 2H), 5.18-4.99 (m, 1H), 4.22-4.04 (m, 2H), 3.10-3.00 (m, 1H), 2.92 (br. s., 4H), 2.43-2.32 (m, 4H), 2.21 (s, 3H), 2.13-1.96 (m, 2H), 1.83 (d, J=12.72 Hz, 1H), 1.74-1.52 (m, 2H), 1.14-1.03 (m, 5H), 0.89-0.80 (m, 2H).

### Example D-189: Synthesis of 5-[(5S)-5-(4-cyclopropylbenzamido)-3,3-difluoropiperidin-1-yl]-3-[(1-methyl-1H-pyrazol-4-yl)amino]pyrazine-2-carboxamide (D-189)

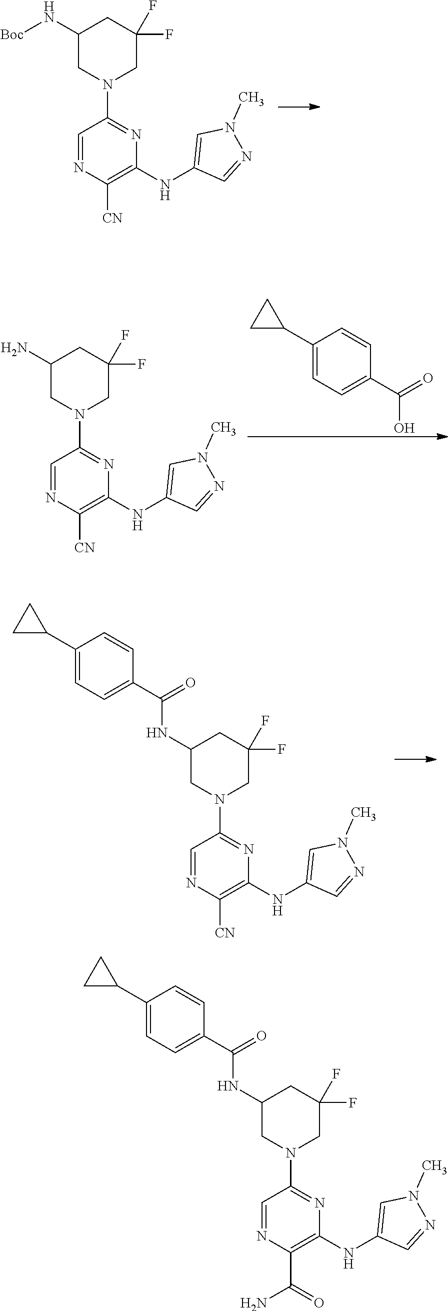 [SMILES: Cn1cc(Nc2nc(N3CC(NC(=O)OC(C)(C)C)CC(F)(F)C3)cnc2C#N)cn1 ; Cn1cc(Nc2nc(N3CC(N)CC(F)(F)C3)cnc2C#N)cn1 ; O=C(O)c1ccc(C2CC2)cc1 ; Cn1cc(Nc2nc(N3CC(NC(=O)c4ccc(C5CC5)cc4)CC(F)(F)C3)cnc2C#N)cn1 ; Cn1cc(Nc2nc(N3CC(NC(=O)c4ccc(C5CC5)cc4)CC(F)(F)C3)cnc2C(N)=O)cn1]

In a similar manner as described in Example 1, tert-butyl N-(1-{5-cyano-6-[(1-methyl-1H-pyrazol-4-yl)amino]pyrazin-2-yl}-5,5-difluoropiperidin-3-yl)carbamate was prepared. MS found for C14H17F2ClN8 as (M+H)⁺ 335.3.

To a solution of tert-butyl N-(1-{5-cyano-6-[(1-methyl-1H-pyrazol-4-yl)amino]pyrazin-2-yl}-5,5-difluoropiperidin-3-yl)carbamate (73.0 mg, 0.17 mmol) in DCM (3 mL), 4N HCl in dioxane (0.25 ml, 0.85 mmol) was added. The mixture was stirred at room temperature overnight, then it was evaporated to dryness to give 5-(5-amino-3,3-difluoropiperidin-1-yl)-3-[(1-methyl-1H-pyrazol-4-yl)amino]pyrazine-2-carbonitrile hydrochloride (69.0 mg, quant. yield) as a yellow solid.

To a solution of 5-(5-amino-3,3-difluoropiperidin-1-yl)-3-[(1-methyl-1H-pyrazol-4-yl)amino]pyrazine-2-carbonitrile hydrochloride (69.0 mg, 0.17 mmol) in DMF (2 mL), DIPEA (1.48 mL, 8.5 mmol), 4-cyclopropylbenzoic acid (34.0 mg, 0.20 mmol) and PyBop (122.0 mg, 0.25 mmol) were added. The mixture was stirred at room temperature for 2 h then it was partitioned between ethyl acetate and H₂O. The organic phase was washed with brine, concentrated and purified by silica NH flash chromatography with 20 to 100% ethyl acetate in cyclohexane then MeOH in ethyl acetate 20% to give N-(1-{5-cyano-6-[(1-methyl-1H-pyrazol-4-yl)amino]pyrazin-2-yl}-5,5-difluoropiperidin-3-yl)-4-cyclopropylbenzamide (111.0 mg, quant. yield). MS found for C24H24F2N8O as (M+H)⁺ 479.5.

To a suspension of N-(1-{5-cyano-6-[(1-methyl-1H-pyrazol-4-yl)amino]pyrazin-2-yl}-5,5-difluoropiperidin-3-yl)-4-cyclopropylbenzamide (111.0 mg, 0.17 mmol), in MeOH/DMSO (2 mL/0.2 mL), TEA (0.4 mL), NaOH (17.0 mg, 0.40 mmol) and H₂O₂ (20.0 μL) were added. The mixture was stirred at room temperature for 3 h, and then it was partitioned between DCM and H₂O. The combined organic phase was dried over Na₂SO₄ filtered and concentrated. The crude was purified by trituration with Et₂O/ethyl acetate 5/1 then submitted to chiral separation to give 5-[(5S)-5-(4-cyclopropylbenzamido)-3,3-difluoropiperidin-1-yl]-3-[(1-methyl-1H-pyrazol-4-yl)amino]pyrazine-2-carboxamide (D-189) (26.5 mg, 31% yield) as yellow solid. MS found for C24H26F2N8O2 as (M+H)⁺ 497.5.

¹H NMR (400 MHz, DMSO) δ 10.89 (s, 1H), 8.51 (d, J=7.43 Hz, 1H), 7.99 (s, 1H), 7.86-7.65 (m, 4H), 7.50 (s, 1H), 7.37 (br. s., 1H), 7.19 (d, J=8.22 Hz, 2H), 4.73 (d, J=10.96 Hz, 2H), 4.17 (d, J=6.26 Hz, 1H), 3.74 (s, 3H), 3.65-3.41 (m, 1H), 3.00 (t, J=12.13 Hz, 1H), 2.56-2.44 (m, 1H), 2.42-2.21 (m, 1H), 2.09-1.90 (m, 1H), 1.09-0.93 (m, 2H), 0.84-0.67 (m, 2H).

### Example D-190: Synthesis of 5-[(5S)-5-(4-cyclopropylbenzamido)-3,3-difluoropiperidin-1-yl]-3-[(1-methyl-1H-pyrazol-4-yl)amino]pyrazine-2-carboxamide (D-190)

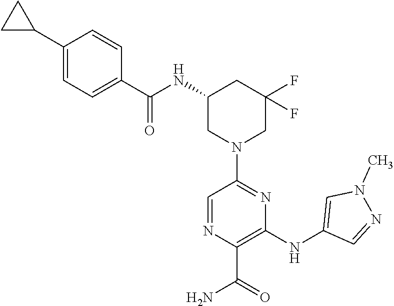 [SMILES: Cn1cc(Nc2nc(N3C[C@H](NC(=O)c4ccc(C5CC5)cc4)CC(F)(F)C3)cnc2C(N)=O)cn1]

In the same experimental procedure as in Example D-189, 5-[(5S)-5-(4-cyclopropylbenzamido)-3,3-difluoropiperidin-1-yl]-3-[(1-methyl-1H-pyrazol-4-yl)amino]pyrazine-2-carboxamide (D-190) was prepared. MS found for C24H26F2N8O2 as (M+H)⁺ 497.5.

¹H NMR (400 MHz, DMSO) δ 10.89 (s, 1H), 8.51 (d, J=7.43 Hz, 1H), 7.99 (s, 1H), 7.88-7.63 (m, 4H), 7.50 (s, 1H), 7.37 (br. s., 1H), 7.19 (d, J=8.22 Hz, 2H), 4.93-4.58 (m, 2H), 4.16 (m, J=6.26 Hz, 1H), 3.74 (s, 3H), 3.70-3.48 (m, 1H), 3.00 (t, J=11.93 Hz, 1H), 2.57-2.43 (m, 1H), 2.44-2.18 (m, 1H), 2.05-1.89 (m, 1H), 1.07-0.94 (m, 2H), 0.81-0.67 (m, 2H).

### Example D-191: Synthesis of 5-[(3R)-3-(4-cyclopropylbenzamido)piperidin-1-yl]-3-[(1-methyl-1H-pyrazol-4-yl)amino]pyrazine-2-carboxamide (D-191)

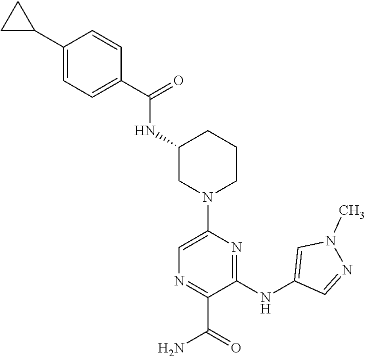 [SMILES: Cn1cc(Nc2nc(N3CCC[C@@H](NC(=O)c4ccc(C5CC5)cc4)C3)cnc2C(N)=O)cn1]

In a similar manner as described in Example D-216 5-[(3R)-3-(4-cyclopropylbenzamido)piperidin-1-yl]-3-[(1-methyl-1H-pyrazol-4-yl)amino]pyrazine-2-carboxamide (D-191) was prepared. MS found for C14H20N8O as (M+H)⁺ 317.1.

¹H NMR (400 MHz, DMSO) δ 10.85 (s, 1H), 8.31 (d, J=7.24 Hz, 1H), 7.99 (s, 1H), 7.83-7.73 (m, 2H), 7.68 (br. s., 1H), 7.60 (s, 1H), 7.48 (s, 1H), 7.26 (br. s., 1H), 7.21-7.11 (m, 2H), 4.57 (d, J=10.96 Hz, 1H), 4.19 (d, J=12.91 Hz, 1H), 4.00-3.87 (m, 1H), 3.74 (s, 3H), 3.11 (t, J=11.05 Hz, 1H), 3.04-2.93 (m, 1H), 2.04-1.92 (m, 2H), 1.92-1.82 (m, 1H), 1.82-1.68 (m, 1H), 1.65-1.49 (m, 1H), 1.06-0.94 (m, 2H), 0.78-0.66 (m, 2H).

### Example D-192: Synthesis of 5-[(3R)-3-(4-cyclopropylbenzamido)-4,4-difluoropiperidin-1-yl]-3-[(1-methyl-1H-pyrazol-4-yl)amino]pyrazine-2-carboxamide (D-192)

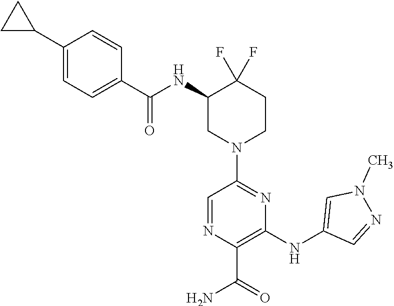 [SMILES: Cn1cc(Nc2nc(N3CCC(F)(F)[C@H](NC(=O)c4ccc(C5CC5)cc4)C3)cnc2C(N)=O)cn1]

In a similar manner as described in Example D-189 5-[(3R)-3-(4-cyclopropylbenzamido)-4,4-difluoropiperidin-1-yl]-3-[(1-methyl-1H-pyrazol-4-yl)amino]pyrazine-2-carboxamide (D-192) was prepared. MS found for C24H26F2N8O2 as (M+H)⁺ 497.5.

¹H NMR (400 MHz, CDCl₃) δ 10.56 (s, 1H), 7.82 (s, 1H), 7.69 (d, J=8.11 Hz, 2H), 7.57 (s, 1H), 7.52 (s, 1H), 7.43 (br. s., 1H), 7.15 (d, J=8.33 Hz, 2H), 6.41 (d, J=8.11 Hz, 1H), 5.27-5.16 (m, 1H), 4.76 (d, J=13.59 Hz, 1H), 4.65-4.50 (m, 1H), 4.37 (d, J=13.37 Hz, 1H), 3.88 (s, 3H), 3.42-3.29 (m, 1H), 3.19-3.08 (m, 1H), 2.42-2.29 (m, 1H), 2.24-2.07 (m, 1H), 2.02-1.89 (m, 1H), 1.12-1.03 (m, 2H), 0.82-0.72 (m, 2H).

### Example D-193: Synthesis of 5-[(3S)-3-(4-cyclopropylbenzamido)-4,4-difluoropiperidin-1-yl]-3-[(1-methylpyrazol-4-yl)amino]pyrazine-2-carboxamide (D-193)

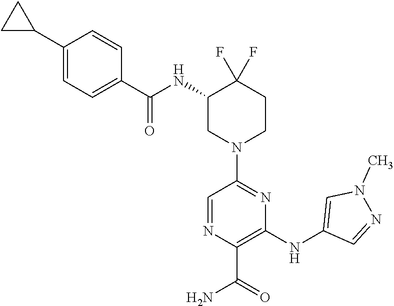 [SMILES: Cn1cc(Nc2nc(N3CCC(F)(F)[C@@H](NC(=O)c4ccc(C5CC5)cc4)C3)cnc2C(N)=O)cn1]

In a similar manner as described in Example D-189 5-[(3S)-3-(4-cyclopropylbenzamido)-4,4-difluoropiperidin-1-yl]-3-[(1-methylpyrazol-4-yl)amino]pyrazine-2-carboxamide (D-193) was prepared. MS found for C24H26F2N8O2 as (M+H)⁺ 497.5.

¹H NMR (400 MHz, CDCl₃) δ 10.60 (s, 1H), 7.88 (s, 1H), 7.69 (d, J=8.33 Hz, 2H), 7.59 (s, 1H), 7.53 (s, 1H), 7.44 (br. s., 1H), 7.15 (d, J=8.33 Hz, 2H), 6.42 (d, J=7.45 Hz, 1H), 5.24 (br. s., 1H), 4.79 (d, J=13.37 Hz, 1H), 4.65-4.47 (m, 1H), 4.36 (d, J=15.79 Hz, 1H), 3.92 (s, 3H), 3.44-3.32 (m, 1H), 3.18-3.07 (m, 1H), 2.46-2.29 (m, 1H), 2.25-2.07 (m, 1H), 1.99-1.92 (m, 1H), 1.11-1.02 (m, 2H), 0.83-0.73 (m, 2H).

### Example D-194: Synthesis of 5-[(2R,3R)-3-[4-(2-hydroxypropan-2-yl)benzamido]-2-methylpiperidin-1-yl]-3-[(1-methyl-1H-pyrazol-4-yl)amino]pyrazine-2-carboxamide (D-194)

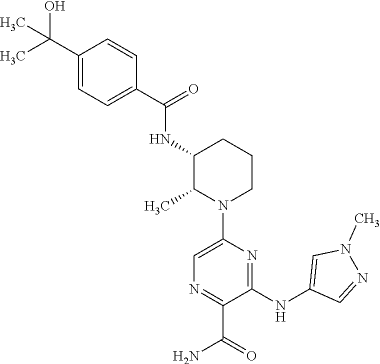 [SMILES: C[C@@H]1[C@H](NC(=O)c2ccc(C(C)(C)O)cc2)CCCN1c1cnc(C(N)=O)c(Nc2cnn(C)c2)n1]

In a similar manner as described in Example D-216 5-[(2R,3R)-3-[4-(2-hydroxypropan-2-yl)benzamido]-2-methylpiperidin-1-yl]-3-[(1-methyl-1H-pyrazol-4-yl)amino]pyrazine-2-carboxamide (D-194) using 4-(2-hydroxypropan-2-yl)benzoic acid (WO2013041535) was prepared. MS found for C25H32N8O3 as (M+H)⁺ 493.0.

¹H NMR (400 MHz, DMSO) δ 10.87 (s, 1H), 8.36 (d, J=6.65 Hz, 1H), 8.04 (s, 1H), 7.86 (d, J=8.22 Hz, 2H), 7.69 (br. s., 1H), 7.60-7.52 (m, 3H), 7.47 (s, 1H), 7.27 (br. s., 1H), 5.48-5.17 (m, 1H), 5.12 (s, 1H), 4.23-3.95 (m, 2H), 3.77 (s, 3H), 3.09 (t, J=12.13 Hz, 1H), 2.04-1.82 (m, 2H), 1.78-1.52 (m, 2H), 1.44 (s, 6H), 1.09 (d, J=7.04 Hz, 3H).

### Example D-195: Synthesis of 3-[(3R)-3-(4-cyclopropylbenzamido)piperidin-1-yl]-5-[(1-methyl-1H-pyrazol-4-yl)amino]-1,2,4-triazine-6-carboxamide (D-195)

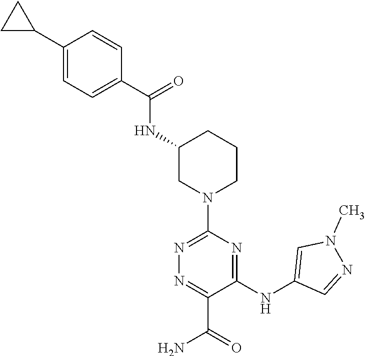 [SMILES: Cn1cc(Nc2nc(N3CCC[C@@H](NC(=O)c4ccc(C5CC5)cc4)C3)nnc2C(N)=O)cn1]

In a similar manner as described in Example D-277 3-[(3R)-3-(4-cyclopropylbenzamido)piperidin-1-yl]-5-[(1-methyl-1H-pyrazol-4-yl)amino]-1,2,4-triazine-6-carboxamide (D-195) was prepared. MS found for C23H27N9O2 as (M+H)⁺ 462.3.

¹H NMR (500 MHz, DMSO) δ 11.00 (s, 1H), 8.36-8.21 (m, 3H), 7.88-7.53 (m, 4H), 7.15 (d, J=8.23 Hz, 2H), 4.72 (br. s., 2H), 3.99-3.88 (m, 1H), 3.83 (s, 3H), 3.24-3.11 (m, 2H), 2.07-1.84 (m, 4H), 1.64-1.51 (m, 1H), 1.00 (dd, J=8.23, 2.20 Hz, 2H), 0.73 (dd, J=4.94, 1.65 Hz, 2H).

### Example D-196: Synthesis of 5-[(2R,3R)-3-[4-(dimethylamino)benzamido]-2-methylpiperidin-1-yl]-3-{[4-(4-methylpiperazin-1-yl)phenyl]amino}pyrazine-2-carboxamide (D-196)

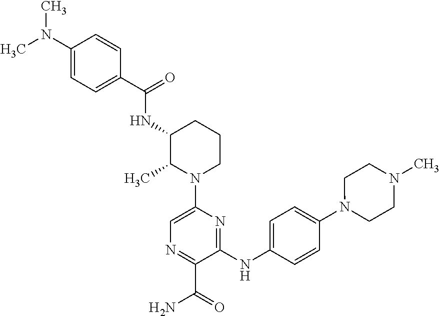

In a similar manner as described in Example 7, 5-[(2R,3R)-3-[4-(dimethylamino)benzamido]-2-methylpiperidin-1-yl]-3-{[4-(4-methylpiperazin-1-yl)phenyl]amino}pyrazine-2-carboxamide (D-196) was prepared. MS found for C31H41N9O2 as (M+H)⁺ 572.3.

¹H NMR (500 MHz, DMSO) δ 10.93 (br. s., 1H), 8.05 (d, J=7.58 Hz, 1H), 7.84 (d, J=8.80 Hz, 2H), 7.70 (br. s., 1H), 7.58 (s, 1H), 7.44 (d, J=8.80 Hz, 2H), 7.27 (br. s., 1H), 6.85-6.68 (m, 4H), 5.12 (br. s., 1H), 4.36-3.92 (m, 2H), 3.18-2.77 (m, 11H), 2.44-2.28 (m, 4H), 2.20 (s, 3H), 1.94 (qd, J=12.88, 3.67 Hz, 1H), 1.84 (d, J=12.96 Hz, 1H), 1.71-1.52 (m, 2H), 1.05 (d, J=6.36 Hz, 3H).

### Example D-197: Synthesis of 3-[(1-methyl-1H-pyrazol-4-yl)amino]-5-[(2R,3R)-2-methyl-3-[4-(propan-2-yl)benzamido]piperidin-1-yl]pyrazine-2-carboxamide (D-197)

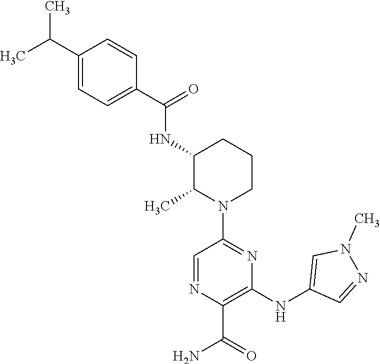 [SMILES: CC(C)c1ccc(C(=O)N[C@@H]2CCCN(c3cnc(C(N)=O)c(Nc4cnn(C)c4)n3)[C@@H]2C)cc1]

In a similar manner as described in Example D-216 3-[(1-methyl-1H-pyrazol-4-yl)amino]-5-[(2R,3R)-2-methyl-3-[4-(propan-2-yl)benzamido]piperidin-1-yl]pyrazine-2-carboxamide (D-197) was prepared. MS found for C25H32N8O2 as (M+H)⁺ 477.3.

¹H NMR (500 MHz, DMSO) δ 10.87 (s, 1H), 8.35 (d, J=6.86 Hz, 1H), 8.03 (br. s., 1H), 7.85 (d, J=8.23 Hz, 2H), 7.69 (br. s., 1H), 7.57 (s, 1H), 7.47 (s, 1H), 7.36 (d, J=8.23 Hz, 2H), 7.28 (br. s., 1H), 5.53-5.10 (m, 1H), 4.25-3.97 (m, 2H), 3.77 (s, 3H), 3.17-3.04 (m, 1H), 2.96 (quin, J=6.86 Hz, 1H), 2.08-1.81 (m, 2H), 1.77-1.50 (m, 2H), 1.23 (d, J=6.86 Hz, 6H), 1.09 (d, J=6.86 Hz, 3H).

### Example D-198: Synthesis of 5-[(2R,3R)-3-(5-cyclopropylpyrimidine-2-amido)-2-methylpiperidin-1-yl]-3-[(1-methyl-1H-pyrazol-4-yl)amino]pyrazine-2-carboxamide (D-198)

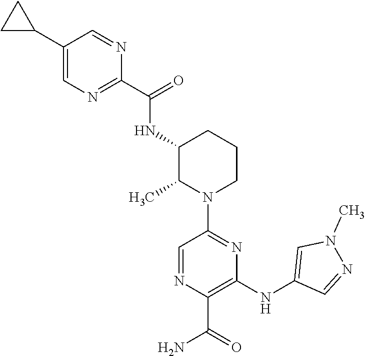 [SMILES: C[C@@H]1[C@H](NC(=O)c2ncc(C3CC3)cn2)CCCN1c1cnc(C(N)=O)c(Nc2cnn(C)c2)n1]

In a similar manner as described in Example D-216, 5-[(2R,3R)-3-(5-cyclopropylpyrimidine-2-amido)-2-methylpiperidin-1-yl]-3-[(1-methyl-1H-pyrazol-4-yl)amino]pyrazine-2-carboxamide (D-198) was prepared. MS found for C23H28N10O2 as (M+H)⁺ 477.5.

¹H NMR (400 MHz, DMSO) δ 10.86 (s, 1H), 8.72 (s, 3H), 8.04 (s, 1H), 7.69 (br. s., 1H), 7.57 (s, 1H), 7.45 (s, 1H), 7.27 (br. s., 1H), 5.32 (br. s., 1H), 4.24-3.96 (m, 2H), 3.82 (s, 3H), 3.17-2.97 (m, 1H), 2.19-1.81 (m, 3H), 1.77-1.52 (m, 2H), 1.16-1.10 (m, 2H), 1.08 (d, J=6.80 Hz, 3H), 0.99-0.93 (m, 2H).

### Example D-199: Synthesis of 5-[2-(4-cyclopropylbenzamido)-8-azabicyclo[3.2.1]octan-8-yl]-3-[(1-methyl-1H-pyrazol-4-yl)amino]pyrazine-2-carboxamide trans enantiomer 1 (D-199)

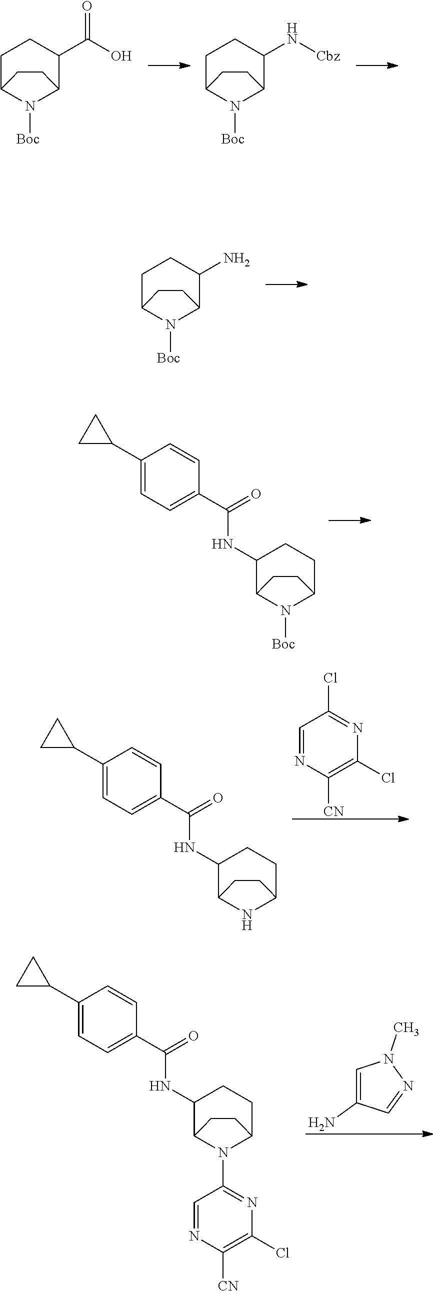

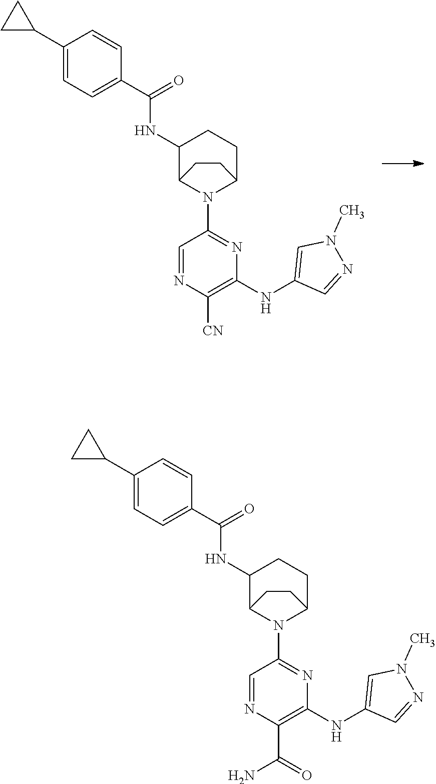 [SMILES: Cn1cc(Nc2nc(N3C4CCC(NC(=O)c5ccc(C6CC6)cc5)C3CC4)cnc2C#N)cn1 ; Cn1cc(Nc2nc(N3C4CCC(NC(=O)c5ccc(C6CC6)cc5)C3CC4)cnc2C(N)=O)cn1]

To a solution of 8-[(tert-butoxy)carbonyl]-8-azabicyclo[3.2.1]octane-2-carboxylic acid (1.456 g, 5.7 mmol) (prepared according to WO2012036997), in toluene (60 mL), TEA (1.6 mL, 11.4 mmol), and DPPA (1.4 mL, 6.0 mmol) were added. The mixture was stirred at 80° C. for 4 h, then benzyl alcohol was added (2.8 mL, 27.0 mmol). The mixture was stirred at 70° C. overnight. Further benzyl alcohol (2.5 mL, 24.1 mmol) was added and the mixture was stirred at 70° C. overnight. Ethyl acetate and H₂O were added; the organic phase was separated, dried over Na₂SO₄, concentrated and purified by C18 reverse phase silica flash chromatography with 0 to 100% ACN 0.1% HCOOH in H₂O to give tert-butyl 2-{[(benzyloxy)carbonyl]amino}-8-azabicyclo[3.2.1]octane-8-carboxylate (1.77 mg, 86% yield) as white solid. MS found for C20H28N2O4 as (M+H)⁺ 361.0.

To a solution of tert-butyl 2-{[(benzyloxy)carbonyl]amino}-8-azabicyclo[3.2.1]octane-8-carboxylate (1.86 g, 5.16 mmol) in MeOH (50 mL), Pd/C (0.4 g) was added. The mixture was stirred under a H₂ atmosphere (1 atm) for 4 h, and then it was filtered. And concentrated to give tert-butyl 2-amino-8-azabicyclo[3.2.1]octane-8-carboxylate (1.19 g, quant. yield) as a colorless oil. MS found for C12H22N2O2 as (M+H)⁺ 227.1.

To a solution of tert-butyl 2-amino-8-azabicyclo[3.2.1]octane-8-carboxylate (1.22 g, 5.3 mmol), in DMF (10 mL), DIPEA (4.5 mL, 25.8 mmol), 4-cyclopropylbenzoic acid (1.0 g, 6.19 mmol) and PyBop (4.0 g, 7.74 mmol) were added. The mixture was stirred at room temperature for 1 h, and then it was partitioned between Et₂O and H₂O. The combined organic phases were dried over Na₂SO₄, concentrated and purified by silica flash chromatography with 0 to 10% ethyl acetate in cyclohexane to give tert-butyl 2-(4-cyclopropylbenzamido)-8-azabicyclo[3.2.1]octane-8-carboxylate (1.75 g, 89% yield). MS found for C22H30N2O3 as (M+H)⁺ 371.1.

To a solution of tert-butyl 2-(4-cyclopropylbenzamido)-8-azabicyclo[3.2.1]octane-8-carboxylate (1.75 g, 4.73 mmol) in DCM (20 mL), 4N HCl in dioxane (4 mL) was added. The mixture was stirred at room temperature for 20 h. The suspension was concentrated to dryness to give N-{8-azabicyclo[3.2.1]octan-2-yl}-4-cyclopropylbenzamide hydrochloride (1.68 g, quant. yield) as a white solid. MS found for C17H22N20Cl as (M+H)⁺ 271.0.

To a solution of N-{8-azabicyclo[3.2.1]octan-2-yl}-4-cyclopropylbenzamide hydrochloride (1.68 g, 4.73 mmol) in DMF (15 mL) DIPEA (3.3 mL, 18.9 mmol), and 3,5-dichloropyrazine-2-carbonitrile (0.93 g, 5.2 mmol) were added. The mixture was stirred at room temperature for 2 h then it was partitioned between ethyl acetate and H₂O. The combined organic phases were dried over Na₂SO₄, and evaporated to dryness to give N-[8-(6-chloro-5-cyanopyrazin-2-yl)-8-azabicyclo[3.2.1]octan-2-yl]-4-cyclopropylbenzamide (2.0 g, quant. yield) as an orange solid. MS found for C22H22ClN5O as (M+H)⁺ 408.0, 410.0.

To a solution of N-[8-(6-chloro-5-cyanopyrazin-2-yl)-8-azabicyclo[3.2.1]octan-2-yl]-4-cyclopropylbenzamide (1.0 g, 2.36 mmol) in dioxane (15 mL), Cs₂CO₃ (3.1 g, 9.44 mmol), 1-methyl-1H-pyrazol-4-amine (0.58 g, 4.3 mmol), (+/−) BINAP (0.25 g, 0.40 mmol) and Pd(OAc)₂ (0.11 g, 0.47 mmol) were added. The mixture was stirred at 90° C. for 2 h, the H₂O (2 mL) was added. The mixture was stirred at 95° C. for 4 h. The suspension was concentrated and purified by silica flash chromatography with 20 to 100% ethyl acetate in cyclohexane to give N-(8-(5-cyano-6-[(1-methyl-1H-pyrazol-4-yl)amino]pyrazin-2-yl)-8-azabicyclo[3.2.1]octan-2-yl)-4-cyclopropylbenzamide (1.09 g, 98% yield) as an orange solid. MS found for C₂₆H₂₈N₈O as (M+H)⁺ 469.1.

To a solution of N-(8-{5-cyano-6-[(1-methyl-1H-pyrazol-4-yl)amino]pyrazin-2-yl}-8-azabicyclo[3.2.1]octan-2-yl)-4-cyclopropylbenzamide (1.09 g, 2.32 mmol) in MeOH/DMSO (10 mL/1 mL), TEA (6.0 mL), NaOH (0.2 g, 5.0 mmol) and H₂O₂ (0.2 mL) were added. The mixture was stirred at room temperature for 2 h, and then it was partitioned between DCM and H₂O. The combined organic phases were concentrated and purified by silica flash chromatography with 20 to 100% ethyl acetate in cyclohexane, then by C18 reverse phase silica flash chromatography with 0 to 100% ACN 0.1% HCOOH in H₂O to give a product that was triturated with Et₂O/ethyl acetate and submitted to chiral separation to give 5-[2-(4-cyclopropylbenzamido)-8-azabicyclo[3.2.1]octan-8-yl]-3-[(1-methyl-1H-pyrazol-4-yl)amino]pyrazine-2-carboxamide trans enantiomer 1 (41.5 g, 4% yield). MS found for C26H30N8O2 as (M+H)⁺ 487.1.

¹H NMR (500 MHz, DMSO) δ 10.84 (s, 1H), 8.10 (d, J=5.76 Hz, 1H), 7.86 (s, 1H), 7.34 (d, J=7.96 Hz, 2H), 7.47 (br. s., 3H), 7.15-7.04 (m, 1H), 6.96 (d, J=6.59 Hz, 2H), 4.97 (br. s., 1H), 4.71 (br. s., 1H), 3.93 (br. s., 1H), 3.80 (s, 3H), 2.33-2.26 (m, 1H), 2.23-2.10 (m, 1H), 2.07-1.75 (m, 5H), 1.53 (dd, J=14.13, 4.53 Hz, 1H), 1.47 (d, J=7.68 Hz, 1H), 0.93 (dd, J=8.23, 1.92 Hz, 2H), 0.68-0.59 (m, 2H).

### Example D-200: Synthesis of 5-[2-(4-cyclopropylbenzamido)-8-azabicyclo[3.2.1]octan-8-yl]-3-[(1-methyl-1H-pyrazol-4-yl)amino]pyrazine-2-carboxamide trans enantiomer 2 (D-200)

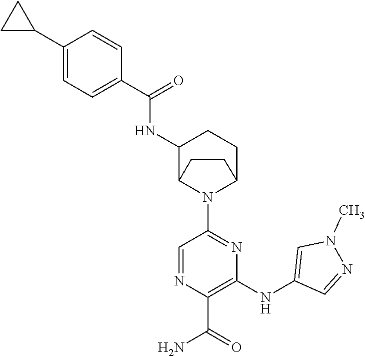 [SMILES: Cn1cc(Nc2nc(N3C4CCC(NC(=O)c5ccc(C6CC6)cc5)C3CC4)cnc2C(N)=O)cn1]

In the same experimental procedure as in Example D-199, 5-[2-(4-cyclopropylbenzamido)-8-azabicyclo[3.2.1]octan-8-yl]-3-[(1-methyl-1H-pyrazol-4-yl)amino]pyrazine-2-carboxamide trans enantiomer 2 (D-200) was prepared. MS found for C26H30N8O2 as (M+H)⁺ 487.1.

¹H NMR (500 MHz, DMSO) δ 10.81 (br. s., 1H), 8.10 (d, J=5.76 Hz, 1H), 7.86 (s, 1H), 7.34 (d, J=7.96 Hz, 2H), 7.67-7.16 (m, 3H), 7.14-7.03 (m, 1H), 6.95 (d, J=6.86 Hz, 2H), 4.98 (br. s., 1H), 4.71 (br. s., 1H), 3.93 (br. s., 1H), 3.80 (s, 4H), 2.32-2.26 (m, 1H), 2.22-2.10 (m, 1H), 2.04-1.72 (m, 5H), 1.53 (dd, J=14.41, 4.25 Hz, 1H), 1.47 (d, J=8.51 Hz, 1H), 0.93 (dd, J=7.96, 1.92 Hz, 2H), 0.64 (d, J=3.84 Hz, 2H).

### Example D-201: Synthesis of 5-[2-(4-cyclopropylbenzamido)-8-azabicyclo[3.2.1]octan-8-yl]-3-[(1-methyl-1H-pyrazol-4-yl)amino]pyrazine-2-carboxamide cis enantiomer 1 (D-201)

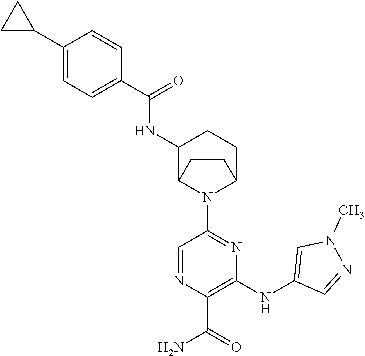 [SMILES: Cn1cc(Nc2nc(N3C4CCC(NC(=O)c5ccc(C6CC6)cc5)C3CC4)cnc2C(N)=O)cn1]

In the same experimental procedure as in Example D-199, 5-[2-(4-cyclopropylbenzamido)-8-azabicyclo[3.2.1]octan-8-yl]-3-[(1-methyl-1H-pyrazol-4-yl)amino]pyrazine-2-carboxamide cis enantiomer 1 (D-201) was prepared. MS found for C26H30N8O2 as (M+H)⁺ 487.1.

¹H NMR (500 MHz, DMSO) δ 11.15-10.64 (m, 1H), 8.22 (br. s., 2H), 7.80 (d, J=7.34 Hz, 2H), 7.98-7.20 (m, 4H), 7.15 (d, J=7.83 Hz, 2H), 4.63 (br. s., 1H), 4.60 (br. s, 1H), 4.29-3.95 (m, 1H), 3.77 (d, J=1.96 Hz, 3H), 2.16 (br. s., 1H), 2.08-1.90 (m, 3H), 1.89-1.65 (m, 4H), 1.61 (d, J=9.78 Hz, 1H), 1.04-0.94 (m, 2H), 0.73 (d, J=3.42 Hz, 2H).

### Example D-202: Synthesis of 5-[2-(4-cyclopropylbenzamido)-8-azabicyclo[3.2.1]octan-8-yl]-3-[(1-methyl-1H-pyrazol-4-yl)amino]pyrazine-2-carboxamide cis enantiomer 2 (D-202)

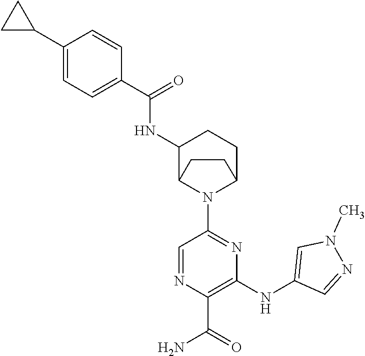 [SMILES: Cn1cc(Nc2nc(N3C4CCC(NC(=O)c5ccc(C6CC6)cc5)C3CC4)cnc2C(N)=O)cn1]

In the same experimental procedure as in Example D-199, 5-[2-(4-cyclopropylbenzamido)-8-azabicyclo[3.2.1]octan-8-yl]-3-[(1-methyl-1H-pyrazol-4-yl)amino]pyrazine-2-carboxamide cis enantiomer 2 (D-202) was prepared. MS found for C26H30N8O2 as (M+H)⁺ 487.1.

¹H NMR (500 MHz, DMSO) δ 10.91 (br. s., 1H), 8.56-8.07 (m, 2H), 7.80 (d, J=7.83 Hz, 2H), 7.95-7.20 (m, 4H), 7.15 (d, J=8.31 Hz, 2H), 4.63 (br. s., 1H), 4.94-4.35 (m, 1H), 4.05 (br. s, 1H), 3.75 (br. s., 3H), 2.17 (br. s., 1H), 2.10-1.91 (m, 3H), 1.89-1.65 (m, 4H), 1.61 (d, J=9.78 Hz, 1H), 0.99 (d, J=1.96 Hz, 2H), 0.78-0.66 (m, 2H).

### Example D-203: Synthesis of 5-[(2R,3R)-3-(6-cyclopropylpyridine-3-amido)-2-methylpiperidin-1-yl]-3-{[4-(4-methylpiperazin-1-yl)phenyl]amino}pyrazine-2-carboxamide (D-203)

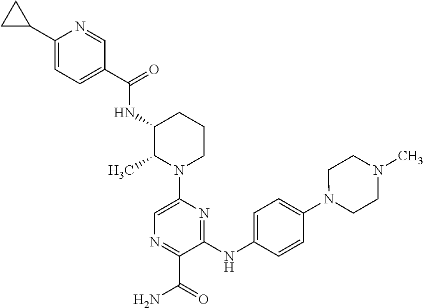 [SMILES: C[C@@H]1[C@H](NC(=O)c2ccc(C3CC3)nc2)CCCN1c1cnc(C(N)=O)c(Nc2ccc(N3CCN(C)CC3)cc2)n1]

In a similar manner as described in Example D-216 5-[(2R,3R)-3-(6-cyclopropylpyridine-3-amido)-2-methylpiperidin-1-yl]-3-{[4-(4-methylpiperazin-1-yl)phenyl]amino}pyrazine-2-carboxamide (D-203) was prepared. MS found for C31H39N9O2 as (M+H)⁺ 570.2.

¹H NMR (400 MHz, DMSO) δ 10.93 (s, 1H), 8.90 (s, 1H), 8.51 (d, J=7.56 Hz, 1H), 8.12 (dd, J=8.17, 1.92 Hz, 1H), 7.70 (br. s., 1H), 7.59 (s, 1H), 7.48-7.38 (m, 3H), 7.27 (br. s., 1H), 6.78 (d, J=8.55 Hz, 2H), 5.11 (br. s., 1H), 4.09 (s, 2H), 3.06 (t, J=12.66 Hz, 1H), 2.90 (br. s., 4H), 2.44-2.27 (m, 4H), 2.25-2.10 (m, 4H), 2.03-1.77 (m, 2H), 1.76-1.48 (m, 2H), 1.20-0.89 (m, 7H).

### Example D-204: Synthesis of 5-[(2R,3R)-3-(4-tert-butylbenzamido)-2-methylpiperidin-1-yl]-3-{[4-(4-methylpiperazin-1-yl)phenyl]amino}pyrazine-2-carboxamide (D-204)

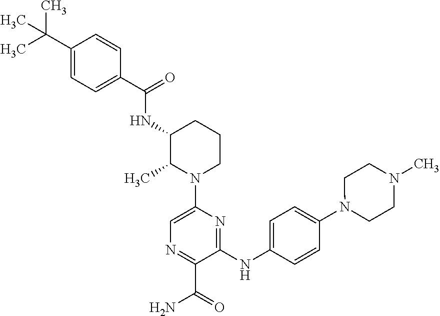 [SMILES: C[C@@H]1[C@H](NC(=O)c2ccc(C(C)(C)C)cc2)CCCN1c1cnc(C(N)=O)c(Nc2ccc(N3CCN(C)CC3)cc2)n1]

In a similar manner as described in Example D-216 5-[(2R,3R)-3-(4-tert-butylbenzamido)-2-methylpiperidin-1-yl]-3-{[4-(4-methylpiperazin-1-yl)phenyl]amino}pyrazine-2-carboxamide (D-204) was prepared. MS found for C33H44N8O2 as (M+H)⁺ 585.4.

¹H NMR (400 MHz, DMSO) δ 10.93 (s, 1H), 8.36 (d, J=7.45 Hz, 1H), 7.89 (d, J=8.11 Hz, 2H), 7.70 (br. s., 1H), 7.59 (s, 1H), 7.51 (d, J=8.33 Hz, 2H), 7.45 (d, J=8.99 Hz, 2H), 7.30-7.23 (m, 1H), 6.81 (d, J=8.77 Hz, 2H), 5.14 (br. s., 1H), 4.09 (m, J=4.80 Hz, 2H), 3.14-3.00 (m, 1H), 2.93 (d, J=3.73 Hz, 4H), 2.43-2.29 (m, 4H), 2.19 (s, 3H), 2.03-1.80 (m, 2H), 1.75-1.52 (m, 2H), 1.32 (s, 9H), 1.06 (d, J=6.80 Hz, 3H).

### Example D-205: Synthesis of 5-[(2R,3R)-3-(5-cyclopropylpyrazine-2-amido)-2-methylpiperidin-1-yl]-3-[(1-methyl-1H-pyrazol-4-yl)amino]pyrazine-2-carboxamide (D-205)

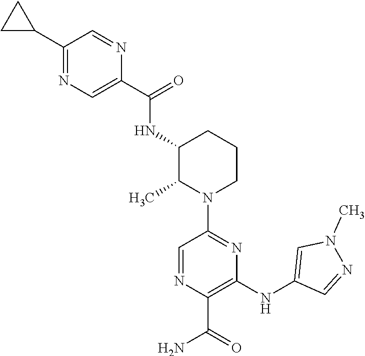 [SMILES: C[C@@H]1[C@H](NC(=O)c2cnc(C3CC3)cn2)CCCN1c1cnc(C(N)=O)c(Nc2cnn(C)c2)n1]

In a similar manner as described in Example D-216 5-[(2R,3R)-3-(5-cyclopropylpyrazine-2-amido)-2-methylpiperidin-1-yl]-3-[(1-methyl-1H-pyrazol-4-yl)amino]pyrazine-2-carboxamide (D-205) was prepared. MS found for C23H28N10O2 as (M+H)⁺ 477.2.

¹H NMR (500 MHz, DMSO) δ 10.95-10.81 (m, 1H), 9.02 (d, J=0.98 Hz, 1H), 8.79-8.67 (m, 2H), 8.01 (s, 1H), 7.70 (br. s., 1H), 7.57 (s, 1H), 7.47 (s, 1H), 7.29 (br. s., 1H), 5.30 (br. s., 1H), 4.21-3.96 (m, 2H), 3.80 (s, 3H), 3.14-2.98 (m, 1H), 2.41-2.30 (m, 1H), 2.15-2.00 (m, 1H), 1.90-1.82 (m, 2H), 1.72 (d, J=9.78 Hz, 1H), 1.67-1.55 (m, 1H), 1.15 (dd, J=8.07, 2.69 Hz, 2H), 1.12-1.04 (m, 5H).

### Example D-206: Synthesis of 3-[(3R)-3-(6-cyclopropyl-1-oxo-1,2-dihydroisoquinolin-2-yl)piperidin-1-yl]-5-[(1-methyl-1H-pyrazol-4-yl)amino]-1,2,4-triazine-6-carboxamide (D-206)

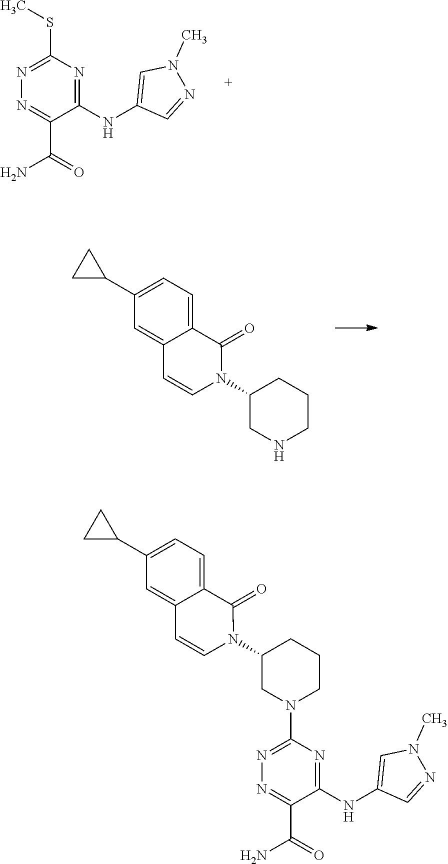 [SMILES: CSc1nnc(C(N)=O)c(Nc2cnn(C)c2)n1 ; O=c1c2ccc(C3CC3)cc2ccn1[C@@H]1CCCNC1 ; Cn1cc(Nc2nc(N3CCC[C@@H](n4ccc5cc(C6CC6)ccc5c4=O)C3)nnc2C(N)=O)cn1]

In a similar manner as described in Example D-165 6-cyclopropyl-2-[(3R)-piperidin-3-yl]-1,2-dihydroisoquinolin-1-one was prepared. MS found for C22H28N2O3 as (M+H)⁺ 369.09. To 5-[(1-methyl-1H-pyrazol-4-yl)amino]-3-(methylsulfanyl)-1,2,4-triazine-6-carboxamide (250.0 mg, 0.94 mmol) suspended in NMP (7 mL) was added m-CPBA (490.0 mg, 2.8 mmol). The mixture was stirred at room temperature for 1 then DIPEA (490 μL, 2.82 mmol) was added followed by 6-cyclopropyl-2-[(3R)-piperidin-3-yl]-1,2-dihydroisoquinolin-1-one (300.0 mg, 1.13 mmol). The mixture was heated at 90° C. for 2 h then let to cool to room temperature. A solid precipitated and was washed with DCM to give 3-[(3R)-3-(6-cyclopropyl-1-oxo-1,2-dihydroisoquinolin-2-yl)piperidin-1-yl]-5-[(1-methyl-1H-pyrazol-4-yl)amino]-1,2,4-triazine-6-carboxamide (131.0 mg, 29% yield). MS found for C25H27N9O2 as (M+H)⁺ 486.5.

¹H NMR (400 MHz, DMSO) δ 10.99 (s, 1H), 8.31 (s, 1H), 8.14 (d, J=8.22 Hz, 1H), 8.04 (br. s., 1H), 7.70 (br. s., 1H), 7.63 (m, J=7.40 Hz, 2H), 7.36 (s, 1H), 7.23 (d, J=8.61 Hz, 1H), 6.63 (d, J=7.43 Hz, 1H), 4.90 (t, J=11.54 Hz, 1H), 5.27-4.44 (m, 2H), 3.62 (br. s., 3H), 3.34-2.99 (m, 2H), 2.12-2.01 (m, 1H), 2.30-1.59 (m, 4H), 1.07 (dd, J=8.41, 2.15 Hz, 2H), 0.88-0.76 (m, 2H).

### Example D-207: Synthesis of 5-[(2R,3R)-3-(4-cyclopropylbenzamido)-2-methylpiperidin-1-yl]-3-[(1-methyl-1H-pyrazol-4-yl)amino]pyridine-2-carboxamide (D-207)

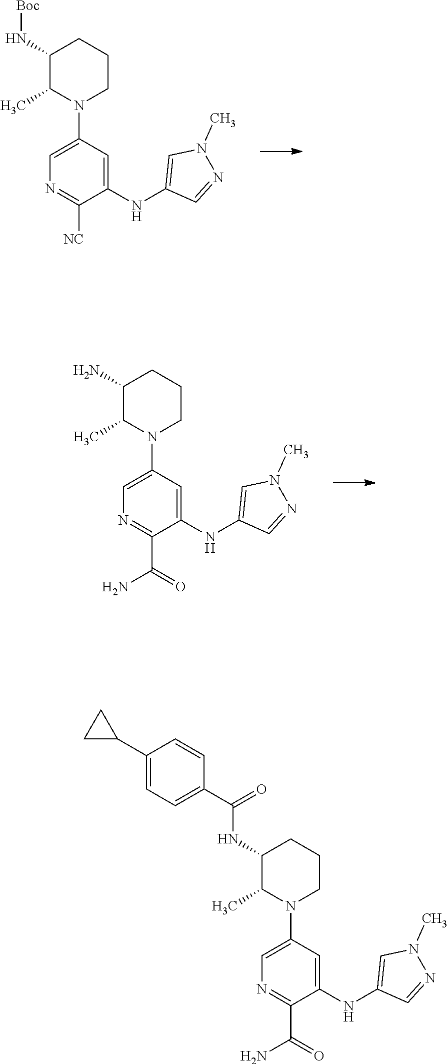 [SMILES: C[C@@H]1[C@H](NC(=O)OC(C)(C)C)CCCN1c1cnc(C#N)c(Nc2cnn(C)c2)c1 ; C[C@@H]1[C@H](N)CCCN1c1cnc(C(N)=O)c(Nc2cnn(C)c2)c1 ; C[C@@H]1[C@H](NC(=O)c2ccc(C3CC3)cc2)CCCN1c1cnc(C(N)=O)c(Nc2cnn(C)c2)c1]

In a similar manner as described in Example D-127 tert-butyl N-[(2R,3R)-1-{6-cyano-5-[(1-methyl-1H-pyrazol-4-yl)amino]pyridin-3-yl}-2-methylpiperidin-3-yl]carbamate was prepared. MS found for C21H29N7O2 as (M+H)⁺ 412.1.

Tert-butyl N-[(2R,3R)-1-{6-cyano-5-[(1-methyl-1H-pyrazol-4-yl)amino]pyridin-3-yl}-2-methylpiperidin-3-yl]carbamate (100.0 mg, 0.24 mmol) was dissolved in TFA (2 mL) then H₂SO₄ (200 μL) was added. The mixture was stirred at room temperature for 2 h. TFA was evaporated and the residue purified through SCX cartridge eluting with NH₃ 7 N in MeOH. The solution was concentrated in vacuo to give 5-[(2R,3R)-3-amino-2-methylpiperidin-1-yl]-3-[(1-methyl-1H-pyrazol-4-yl)amino]pyridine-2-carboxamide (30.0 mg, 38% yield). MS found for C16H23N7O as (M+H)⁺ 330.1.

5-[(2R,3R)-3-amino-2-methylpiperidin-1-yl]-3-[(1-methyl-1H-pyrazol-4-yl)amino]pyridine-2-carboxamide (30.0 mg, 0.09 mmol) was dissolved in DMF (3 mL), 4-cyclopropylbenzoic acid (20.0 mg, 0.12 mmol) was added followed by PyBop (63.0 mg, 0.12 mmol) and DIPEA (73.9 μL, 0.46 mmol). The mixture was stirred at room temperature for 1 h. DMF was evaporated and the residue purified by preparative HPLC to give 5-[(2R,3R)-3-(4-cyclopropylbenzamido)-2-methylpiperidin-1-yl]-3-[(1-methyl-1H-pyrazol-4-yl)amino]pyridine-2-carboxamide (20.4 mg, 47% yield). MS found for C26H31N7O2 as (M+H)⁺ 474.2.

¹H NMR (500 MHz, DMSO) δ 9.97 (s, 1H), 8.26 (d, J=7.00 Hz, 1H), 7.85 (s, 1H), 7.80-7.74 (m, 3H), 7.66 (d, J=2.47 Hz, 1H), 7.42 (s, 1H), 7.22 (br. s., 1H), 7.16 (d, J=8.37 Hz, 2H), 6.62 (d, J=2.33 Hz, 1H), 4.42-4.30 (m, 1H), 4.04 (td, J=12.04, 4.60 Hz, 1H), 3.83 (s, 3H), 3.53 (d, J=12.49 Hz, 1H), 3.04-2.91 (m, 1H), 2.04-1.94 (m, 1H), 1.93-1.82 (m, 1H), 1.76 (d, J=13.04 Hz, 1H), 1.68-1.49 (m, 2H), 1.08-0.96 (m, 5H), 0.78-0.70 (m, 2H).

### Example D-208: Synthesis of 5-[(2R,3R)-3-(5-tert-butylpyridine-2-amido)-2-methylpiperidin-1-yl]-3-[(1-methyl-1H-pyrazol-4-yl)amino]pyrazine-2-carboxamide (D-208)

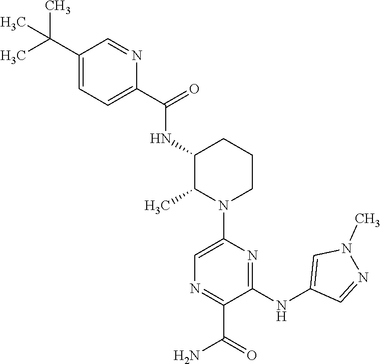 [SMILES: C[C@@H]1[C@H](NC(=O)c2ccc(C(C)(C)C)cn2)CCCN1c1cnc(C(N)=O)c(Nc2cnn(C)c2)n1]

In a similar manner as described in Example D-216 5-[(2R,3R)-3-(5-tert-butylpyridine-2-amido)-2-methylpiperidin-1-yl]-3-[(1-methyl-1H-pyrazol-4-yl)amino]pyrazine-2-carboxamide (D-208) was prepared. MS found for C25H33N9O2 as (M+H)⁺ 492.2.

¹H NMR (500 MHz, DMSO) δ 10.88 (s, 1H), 8.98 (d, J=2.47 Hz, 1H), 8.56 (d, J=6.86 Hz, 1H), 8.18 (dd, J=8.23, 2.47 Hz, 1H), 8.03 (br. s., 1H), 7.70 (br. s., 1H), 7.60-7.54 (m, 2H), 7.48 (s, 1H), 7.29 (br. s., 1H), 5.50-5.14 (m, 1H), 4.21-3.98 (m, 2H), 3.78 (s, 3H), 3.13-3.04 (m, 1H), 2.01-1.81 (m, 2H), 1.78-1.54 (m, 2H), 1.34 (s, 9H), 1.10 (d, J=6.86 Hz, 3H).

### Example D-209: Synthesis of 3-[(1-methyl-1H-pyrazol-4-yl)amino]-5-[(3R)-3-(3-methyl-2-oxoimidazolidin-1-yl)piperidin-1-yl]pyrazine-2-carboxamide (D-209)

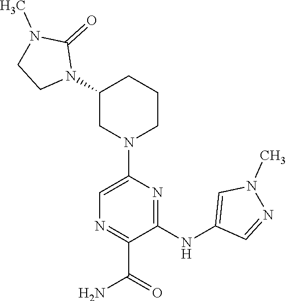 [SMILES: CN1CCN([C@@H]2CCCN(c3cnc(C(N)=O)c(Nc4cnn(C)c4)n3)C2)C1=O]

In a similar manner as described in Example 52, 3-[(1-methyl-1H-pyrazol-4-yl)amino]-5-[(3R)-3-(3-methyl-2-oxoimidazolidin-1-yl)piperidin-1-yl]pyrazine-2-carboxamide (D-209) was prepared. MS found for C18H25N9O2 as (M+H)⁺ 400.1.

¹H NMR (500 MHz, MeOD) δ 7.91 (s, 1H), 7.57 (s, 1H), 7.51 (s, 1H), 4.54 (d, J=1.25 Hz, 1H), 4.31 (d, J=12.90 Hz, 1H), 3.86 (s, 3H), 3.77 (tt, J=11.15, 4.22 Hz, 1H), 3.53-3.41 (m, 2H), 3.41-3.33 (m, 2H), 3.12-2.97 (m, 2H), 2.78 (s, 3H), 2.01-1.78 (m, 3H), 1.75-1.58 (m, 1H).

### Example D-210: Synthesis of 5-[(2R,3R)-3-(4-cyclopropylbenzamido)-2-methylpiperidin-1-yl]-3-[(2-methyl-1,3-thiazol-5-yl)amino]pyrazine-2-carboxamide (D-210)

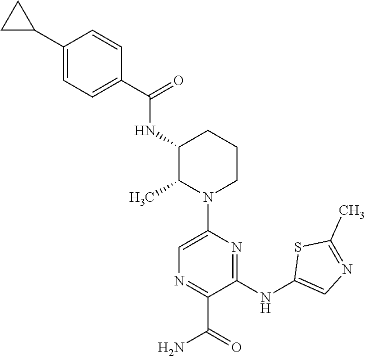 [SMILES: Cc1ncc(Nc2nc(N3CCC[C@@H](NC(=O)c4ccc(C5CC5)cc4)[C@H]3C)cnc2C(N)=O)s1]

In a similar manner as described in Example D-216 5-[(2R,3R)-3-(4-cyclopropylbenzamido)-2-methylpiperidin-1-yl]-3-[(2-methyl-1,3-thiazol-5-yl)amino]pyrazine-2-carboxamide (D-210) was prepared. MS found for C25H29N7O2S as (M+H)⁺ 492.2.

¹H NMR (500 MHz, CDCl₃) δ 11.60-11.29 (m, 1H), 7.68 (d, J=8.23 Hz, 2H), 7.59 (s, 1H), 7.44-7.35 (m, 2H), 7.15 (d, J=8.23 Hz, 2H), 5.94 (d, J=7.14 Hz, 1H), 5.26 (br. s., 2H), 4.44 (br. s., 1H), 4.36-4.24 (m, 1H), 3.19-3.02 (m, 1H), 2.64 (br. s., 3H), 2.07-1.92 (m, 3H), 1.79 (t, J=9.74 Hz, 2H), 1.23 (d, J=6.86 Hz, 3H), 1.12-1.00 (m, 2H), 0.87-0.71 (m, 2H).

### Example D-211: Synthesis of 5-[(2R,3R)-3-(4-cyclopropylbenzamido)-2-methylpiperidin-1-yl]-3-{[4-(octahydroindolizin-7-yloxy)phenyl]amino}pyrazine-2-carboxamide (D-211)

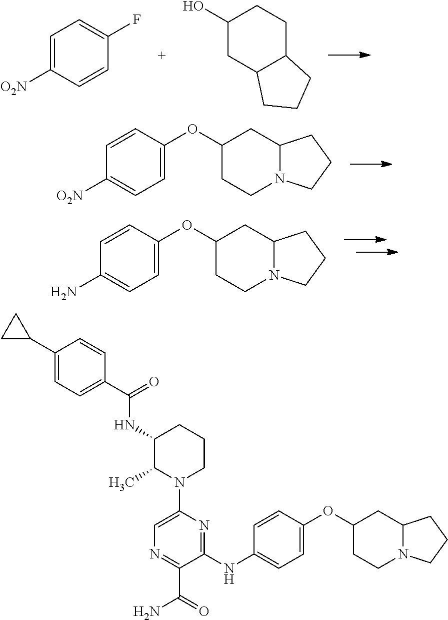

Octahydroindolizin-7-ol (150.0 mg, 1.06 mmol) was dissolved in DMF (10 mL), NaH 60% in mineral oil (80.0 mg, 1.27 mmol) was added portionwise. After 15 min 1-fluoro-4-nitrobenzene (150.0 mg, 1.06 mmol) was added and the reaction was left stirring at 50° C. overnight. Water was added carefully, followed by ethyl acetate. The aqueous phase was extracted with ethyl acetate. The organic phases were dried and concentrated to give 7-(4-nitrophenoxy)-octahydroindolizine (265.0 mg, 95% yield). MS found for C14H18N2O3 as (M+H)⁺ 263.1. 7-(4-Nitrophenoxy)-octahydroindolizine (265.0 mg, 1.01 mmol) was dissolved in EtOH (20 mL), Pd/C (50 mg) was added followed by stirring under H₂ (1 atm) for 2 h. The catalyst was filtered off and the solvent evaporated. 4-(octahydroindolizin-7-yloxy)aniline (211.0 mg, 90% yield) was obtained as a brown oil.

In a similar manner as described in Example D-216 5-[(2R,3R)-3-(4-cyclopropylbenzamido)-2-methylpiperidin-1-yl]-3-{[4-(octahydroindolizin-7-yloxy)phenyl]amino}pyrazine-2-carboxamide (D-211) was prepared using 4-(octahydroindolizin-7-yloxy)aniline. MS found for C₃₅H₄₃N₇O₃ as (M+H)⁺ 610.4.

¹H NMR (400 MHz, CDCl₃) δ 10.68 (s, 1H), 7.68 (d, J=7.83 Hz, 2H), 7.57-7.48 (m, 3H), 7.46-7.37 (m, 1H), 7.15 (d, J=8.22 Hz, 2H), 6.88 (d, J=9.00 Hz, 2H), 5.90 (d, J=7.43 Hz, 1H), 5.21-5.01 (m, 2H), 4.43-4.23 (m, 2H), 4.15 (br. s., 1H), 3.24-2.91 (m, 3H), 2.38-1.34 (m, 15H), 1.19 (d, J=6.65 Hz, 3H), 1.11-1.01 (m, 2H), 0.86-0.74 (m, 2H).

### Example D-212: Synthesis of 5-[(2R,3R)-3-[4-(1-hydroxycyclopentyl)benzamido]-2-methylpiperidin-1-yl]-3-[(1-methyl-1H-pyrazol-4-yl)amino]pyrazine-2-carboxamide (D-212)

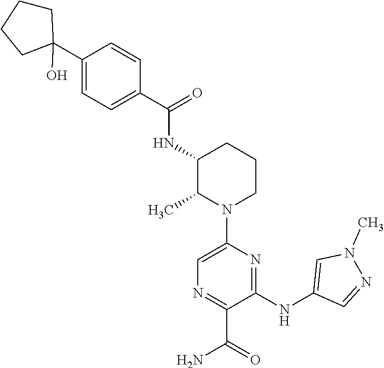 [SMILES: C[C@@H]1[C@H](NC(=O)c2ccc(C3(O)CCCC3)cc2)CCCN1c1cnc(C(N)=O)c(Nc2cnn(C)c2)n1]

In a similar manner as described in Example D-216 5-[(2R,3R)-3-[4-(1-hydroxycyclopentyl)benzamido]-2-methylpiperidin-1-yl]-3-[(1-methyl-1H-pyrazol-4-yl)amino]pyrazine-2-carboxamide (D-212) was prepared staroom temperatureing from 4-(1-hydroxycyclopentyl)benzoic acid (WO20120196869). MS found for C27H34N8O3 as (M+H)⁺ 519.2.

¹H NMR (400 MHz, DMSO) δ 10.87 (s, 1H), 8.36 (d, J=6.53 Hz, 1H), 8.04 (s, 1H), 7.86 (d, J=8.53 Hz, 2H), 7.68 (br. s., 1H), 7.61-7.50 (m, 3H), 7.47 (s, 1H), 7.26 (br. s., 1H), 5.61-5.06 (m, 1H), 4.89 (s, 1H), 4.23-3.96 (m, 2H), 3.77 (s, 3H), 3.09 (t, J=12.55 Hz, 1H), 2.02-1.54 (m, 12H), 1.09 (d, J=6.78 Hz, 3H).

### Example D-213: Synthesis of 3-[(1-methyl-1H-pyrazol-4-yl)amino]-5-[(2R,3R)-2-methyl-3-[4-((1,1,1-trifluoro-2-hydroxypropan-2-yl)benzamido]piperidin-1-yl]pyrazine-2-carboxamide diastereoisomer 1 (D-213)

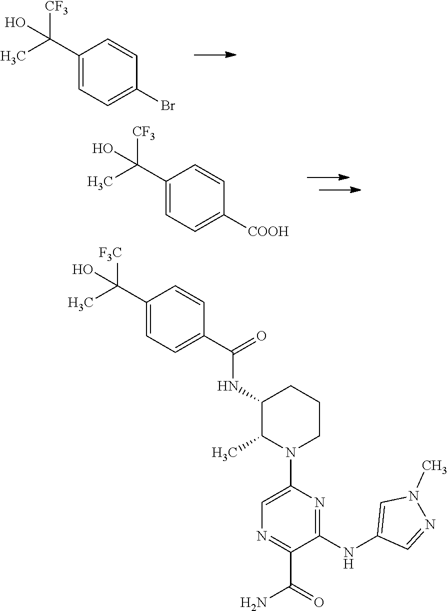

3-[(1-methyl-1H-pyrazol-4-yl)amino]-5-[(2R,3R)-2-methyl-3-[4-(1,1,1-trifluoro-2-hydroxypropan-2-yl)benzamido]piperidin-1-yl]pyrazine-2-carboxamide (150.0 mg, 0.56 mmol) was dissolved in dry THF (5 mL) under N₂. The mixture was cooled to −78° C. then treated with BuLi 2.5 M (446 μL, 1.11 mmol). The mixture was stirred at −78° C. for 20 min then solid CO₂ was poured into the flask. The reaction was let to warm to room temperature, treated with HCl 1N (50 mL) and extracted with ethyl acetate (50 mL×3). The combined organic phases were dried over Na₂SO₄, and concentrated to give 4-(1,1,1-trifluoro-2-hydroxypropan-2-yl)benzoic acid (130.0 mg, quant. yield) as a color less oil. MS found for C10H9F3O3 as (M−H)⁻ 233.2.

In a similar manner as described in Example D-216 3-[(1-methyl-1H-pyrazol-4-yl)amino]-5-[(2R,3R)-2-methyl-3-[4-(1,1,1-trifluoro-2-hydroxypropan-2-yl)benzamido]piperidin-1-yl]pyrazine-2-carboxamide diastereoisomer 1 (D-213) was prepared using 4-(1,1,1-trifluoro-2-hydroxypropan-2-yl)benzoic acid. MS found for C25H29F3N8O3 as (M+H)⁺ 547.1.

¹H NMR (400 MHz, DMSO) δ 10.87 (s, 1H), 8.48 (d, J=6.69 Hz, 1H), 8.03 (s, 1H), 7.92 (d, J=8.44 Hz, 2H), 7.75-7.65 (m, 3H), 7.57 (s, 1H), 7.48 (s, 1H), 7.28 (br. s., 1H), 6.72 (s, 1H), 5.32 (br. s., 1H), 4.26-3.96 (m, 2H), 3.77 (s, 3H), 3.09 (t, J=12.11 Hz, 1H), 2.06-1.81 (m, 2H), 1.79-1.51 (m, 5H), 1.10 (d, J=6.80 Hz, 3H).

### Example D-214: Synthesis of 3-[(1-methyl-1H-pyrazol-4-yl)amino]-5-[(2R,3R)-2-methyl-3-[4-(1,1,1-trifluoro-2-hydroxypropan-2-yl)benzamido]piperidin-1-yl]pyrazine-2-carboxamide diastereoisomer 2 (D-214)

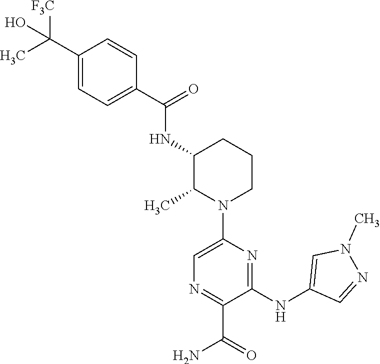 [SMILES: C[C@@H]1[C@H](NC(=O)c2ccc(C(C)(O)C(F)(F)F)cc2)CCCN1c1cnc(C(N)=O)c(Nc2cnn(C)c2)n1]

In the same experimental procedure as in Example D-213, 3-[(1-methyl-1H-pyrazol-4-yl)amino]-5-[(2R,3R)-2-methyl-3-[4-(1,1,1-trifluoro-2-hydroxypropan-2-yl)benzamido]piperidin-1-yl]pyrazine-2-carboxamide diastereoisomer 2 (D-214) was prepared. MS found for C25H29F3N8O3 as (M+H)⁺ 547.1.

¹H NMR (400 MHz, DMSO) δ 10.87 (s, 1H), 8.48 (d, J=6.69 Hz, 1H), 8.03 (s, 1H), 7.92 (d, J=8.44 Hz, 2H), 7.75-7.65 (m, 3H), 7.57 (s, 1H), 7.48 (s, 1H), 7.28 (br. s., 1H), 6.72 (s, 1H), 5.32 (br. s., 1H), 4.26-3.96 (m, 2H), 3.77 (s, 3H), 3.09 (t, J=12.11 Hz, 1H), 2.06-1.81 (m, 2H), 1.79-1.51 (m, 5H), 1.10 (d, J=6.80 Hz, 3H).

### Example D-215: Synthesis of 5-[(2R,3R)-3-(4-cyclopropylbenzamido)-2-methylpiperidin-1-yl]-3-{[1-(2-hydroxy-2-methylpropyl)-1H-pyrazol-4-yl]amino}pyrazine-2-carboxamide (D-215)

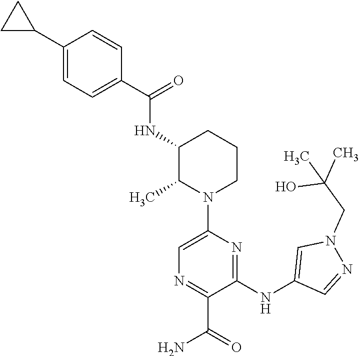 [SMILES: C[C@@H]1[C@H](NC(=O)c2ccc(C3CC3)cc2)CCCN1c1cnc(C(N)=O)c(Nc2cnn(CC(C)(C)O)c2)n1]

In a similar manner as described in Example D-216 5-[(2R,3R)-3-(4-cyclopropylbenzamido)-2-methylpiperidin-1-yl]-3-{[1-(2-hydroxy-2-methylpropyl)-1H-pyrazol-4-yl]amino}pyrazine-2-carboxamide (D-215) was prepared using 1-(4-amino-1H-pyrazol-1-yl)-2-methylpropan-2-ol (WO2015058129). MS found for C28H36N8O3 as (M+H)⁺ 533.2.

¹H NMR (500 MHz, DMSO) δ 10.87 (s, 1H), 8.35 (d, J=6.85 Hz, 1H), 8.05 (s, 1H), 7.81 (d, J=8.07 Hz, 2H), 7.69 (br. s., 1H), 7.57 (s, 1H), 7.48 (s, 1H), 7.28 (br. s., 1H), 7.17 (d, J=8.31 Hz, 2H), 5.22 (br. s., 1H), 4.47 (br. s., 1H), 4.17 (br. s., 1H), 4.05-3.96 (m, 1H), 3.95-3.83 (m, 2H), 3.06 (t, J=12.96 Hz, 1H), 2.03-1.89 (m, 2H), 1.84 (d, J=12.72 Hz, 1H), 1.69 (d, J=10.27 Hz, 1H), 1.65-1.52 (m, 1H), 1.10 (d, J=6.85 Hz, 3H), 1.03-0.98 (m, 2H), 0.88 (br. s., 6H), 0.76-0.68 (m, 2H).

### Example D-216: Synthesis of 5-[(2R,3R)-3-(2,2-difluoro-2H-1,3-benzodioxole-5-amido)-2-methylpiperidin-1-yl]-3-[(1-methyl-1H-pyrazol-4-yl)amino]pyrazine-2-carboxamide (D-216)

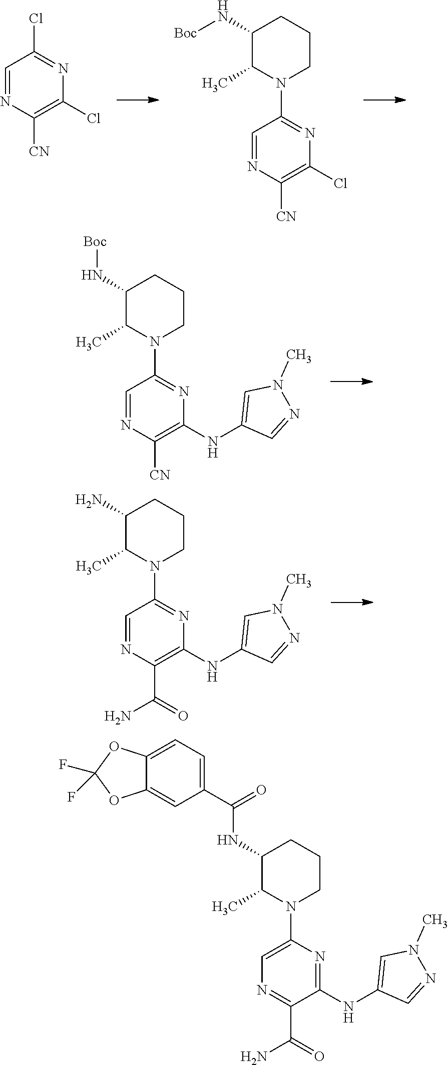

3,5-Dichloropyrazine-2-carbonitrile (6.42 g, 36.9 mmol) and tert-butyl N-[(2R,3R)-2-methylpiperidin-3-yl]carbamate (7.92 g, 36.9 mmol) were dissolved in DMF (60 mL). DIPEA (12.8 mL, 73.8 mmol) was added and the mixture was stirred at room temperature overnight. The reaction mixture was poured into ice then it was extracted with ethyl acetate (3×200 mL). The organic phases were dried over Na₂SO₄, filtered and concentrated. The obtained crude was purified by silica flash chromatography with 0 to 70% ethyl acetate in cyclohexane to afford tert-butyl N-[(2R,3R)-1-(6-chloro-5-cyanopyrazin-2-yl)-2-methylpiperidine-3-yl]carbamate (12.25 g, 94% yield). MS found for C16H22ClN5O2 as (M+H)⁺ 352.4.

To a solution of tert-butyl N-[(2R,3R)-1-(6-chloro-5-cyanopyrazin-2-yl)-2-methylpiperidin-3-yl]carbamate (1.5 g, 4.26 mmol) in 1,4-dioxane (20 mL), Cs₂CO₃ (5.55 g, 17.04 mmol), 1-methyl-1H-pyrazol-4-amine (0.74 g, 7.668 mmol) (+/−) BINAP (0.5 g, 0.85 mmol), Pd(OAc)₂ (0.19 g, 0.852 mmol) and some drops of H₂O were added under nitrogen. The mixture was stirred at 90° C. overnight. Further (+/−) BINAP (0.25 g) and Pd(OAc)2 (0.1 g) were added and the mixture was stirred at 90° C. for other 3 h. It was left to reach room temperature then it was filtered and purified by silica flash chromatography with 20 to 100% ethyl acetate in cyclohexane to give tert-butyl N-[(2R,3R)-1-{5-cyano-6-[(1-methyl-1H-pyrazol-4-yl)amino]pyrazin-2-yl}-2-methylpiperidin-3-yl]carbamate (1.55 g, 88% yield) as a yellow foam. MS found for C20H28N8O2 as (M+H)⁺ 413.1.

To a solution of N-[(2R,3R)-1-{5-cyano-6-[(1-methyl-1H-pyrazol-4-yl)amino]pyrazin-2-yl}-2-methylpiperidin-3-yl]carbamate (1.55 g, 3.76 mmol) in TFA (10 mL) H₂SO₄ (0.4 mL) was added. The mixture was stirred at 40° C. for 3 h then it was concentrated. The obtained crude was purified by SCX cartridge to give 5-[(2R,3R)-3-amino-2-methylpiperidin-1-yl]-3-[(1-methyl-1H-pyrazol-4-yl)amino]pyrazine-2-carboxamide (0.97 g, 78% yield) as a yellow foam. MS found for C₁₅H₂₂N₈O as (M+H)⁺ 331.0

In a similar manner as described in Example 8, 5-[(2R,3R)-3-(2,2-difluoro-2H-1,3-benzodioxole-5-amido)-2-methylpiperidin-1-yl]-3-[(1-methyl-1H-pyrazol-4-yl)amino]pyrazine-2-carboxamide (D-216) was prepared using 2,2-difluoro-2H-1,3-benzodioxole-5-carboxylic acid. MS found for C23H24F2N8O4 as (M+H)⁺ 515.2.

¹H NMR (500 MHz, DMSO) δ 10.87 (s, 1H), 8.51 (d, J=6.86 Hz, 1H), 8.00 (s, 1H), 7.94 (d, J=1.65 Hz, 1H), 7.85 (dd, J=8.51, 1.65 Hz, 1H), 7.70 (br. s., 1H), 7.59-7.52 (m, 2H), 7.50-7.45 (m, 1H), 7.32-7.26 (m, 1H), 5.30 (br. s., 1H), 4.13 (br. s., 1H), 4.03 (td, J=12.01, 4.80 Hz, 1H), 3.76 (s, 3H), 3.14-3.05 (m, 1H), 2.01-1.90 (m, 1H), 1.89-1.82 (m, 1H), 1.77-1.69 (m, 1H), 1.67-1.54 (m, 1H), 1.10 (d, J=6.86 Hz, 3H).

### Example D-217: Synthesis of 5-[(2R,3R)-3-{4-[(2-hydroxyethyl)(methyl)amino]benzamido}-2-methylpiperidin-1-yl]-3-[(1-methyl-1H-pyrazol-4-yl)amino]pyrazine-2-carboxamide (D-217)

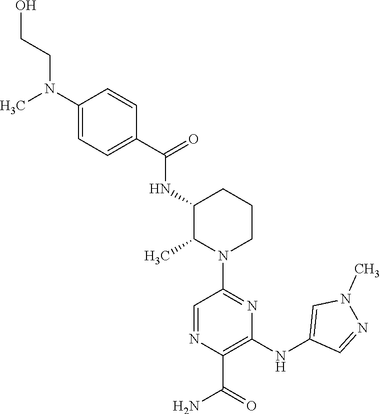 [SMILES: C[C@@H]1[C@H](NC(=O)c2ccc(N(C)CCO)cc2)CCCN1c1cnc(C(N)=O)c(Nc2cnn(C)c2)n1]

In a similar manner as described in Example 8, 5-[(2R,3R)-3-{4-[(2-hydroxyethyl)(methyl)amino]benzamido}-2-methylpiperidin-1-yl]-3-[(1-methyl-1H-pyrazol-4-yl)amino]pyrazine-2-carboxamide (D-217) was prepared using 4-[(2-hydroxyethyl)(methyl)amino]benzoic acid. MS found for C25H33N9O3 as (M+H)⁺ 508.1.

¹H NMR (500 MHz, DMSO) δ 10.84 (s, 1H), 8.04 (br. s., 1H), 8.00 (d, J=6.9 Hz, 1H), 7.77 (d, J=9.1 Hz, 2H), 7.67 (br. s., 1H), 7.54 (s, 1H), 7.44 (s, 1H), 7.26 (br. s., 1H), 6.69 (d, J=9.1 Hz, 2H), 5.31 (br. s., 1H), 4.69 (t, J=5.4 Hz, 1H), 4.09 (br. s., 1H), 3.94-4.01 (m, 1H), 3.76 (s, 3H), 3.50-3.58 (m, 2H), 3.40-3.47 (m, 2H), 3.02-3.10 (m, 1H), 2.97 (s, 3H), 1.89-1.97 (m, 1H), 1.79-1.87 (m, 1H), 1.64-1.72 (m, 1H), 1.52-1.63 (m, 1H), 1.05 (d, J=6.9 Hz, 3H).

### Example D-218: Synthesis of 5-[(2R,3R)-3-[4-(diethylamino)benzamido]-2-methylpiperidin-1-yl]-3-[(1-methyl-1H-pyrazol-4-yl)amino]pyrazine-2-carboxamide (D-218)

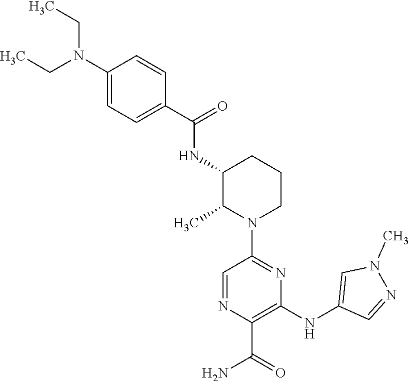 [SMILES: CCN(CC)c1ccc(C(=O)N[C@@H]2CCCN(c3cnc(C(N)=O)c(Nc4cnn(C)c4)n3)[C@@H]2C)cc1]

In a similar manner as described in Example 8, 5-[(2R,3R)-3-[4-(diethylamino)benzamido]-2-methylpiperidin-1-yl]-3-[(1-methyl-1H-pyrazol-4-yl)amino]pyrazine-2-carboxamide (D-218) was prepared using 4-(diethylamino)benzoic acid. MS found for C26H35N9O2 as (M+H)⁺ 506.2.

¹H NMR (500 MHz, DMSO) δ 10.84 (s, 1H), 8.04 (br. s., 1H), 7.97 (d, J=6.9 Hz, 1H), 7.76 (d, J=9.1 Hz, 2H), 7.67 (br. s., 1H), 7.54 (s, 1H), 7.44 (s, 1H), 7.26 (br. s., 1H), 6.65 (d, J=8.8 Hz, 2H), 5.31 (br. s., 1H), 4.09 (br. s., 1H), 3.93-4.02 (m, 1H), 3.77 (s, 3H), 3.38 (q, J=6.9 Hz, 4H), 3.00-3.11 (m, 1H), 1.87-2.00 (m, 1H), 1.80-1.86 (m, 1H), 1.64-1.71 (m, 1H), 1.51-1.63 (m, 1H), 1.09 (t, J=7.0 Hz, 6H), 1.05 (d, J=6.6 Hz, 3H).

### Example D-219: Synthesis of 5-[(2R,3R)-3-(4-cyclopropoxybenzamido)-2-methylpiperidin-1-yl]-3-[(1-methyl-1H-pyrazol-4-yl)amino]pyrazine-2-carboxamide (D-219)

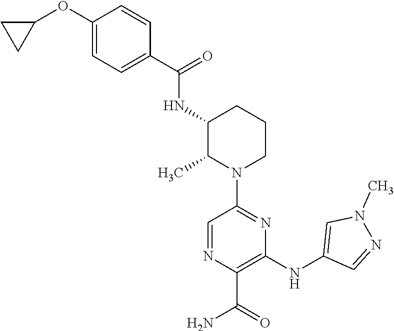 [SMILES: C[C@@H]1[C@H](NC(=O)c2ccc(OC3CC3)cc2)CCCN1c1cnc(C(N)=O)c(Nc2cnn(C)c2)n1]

In a similar manner as described in Example 8, 5-[(2R,3R)-3-(4-cyclopropoxybenzamido)-2-methylpiperidin-1-yl]-3-[(1-methyl-1H-pyrazol-4-yl)amino]pyrazine-2-carboxamide (D-219) was prepared using 4-cyclopropoxybenzoic acid. MS found for C25H30N8O3 as (M+H)⁺ 491.1.

¹H NMR (500 MHz, DMSO) δ 10.85 (s, 1H), 8.27 (d, J=6.9 Hz, 1H), 8.02 (br. s., 1H), 7.90 (d, J=8.8 Hz, 2H), 7.67 (br. s., 1H), 7.55 (s, 1H), 7.45 (s, 1H), 7.26 (br. s., 1H), 7.11 (d, J=8.8 Hz, 2H), 5.30 (br. s., 1H), 4.09 (br. s., 1H), 3.96-4.04 (m, 1H), 3.88-3.93 (m, 1H), 3.75 (s, 3H), 3.02-3.11 (m, 1H), 1.89-1.99 (m, 1H), 1.81-1.87 (m, 1H), 1.66-1.73 (m, 1H), 1.51-1.64 (m, 1H), 1.07 (d, J=6.6 Hz, 3H), 0.77-0.84 (m, 2H), 0.63-0.69 (m, 2H).

### Example D-220: Synthesis of 3-[(1-methyl-1H-pyrazol-4-yl)amino]-5-[(2R,3R)-2-methyl-3-[4-(propan-2-yl)benzenesulfonamido]piperidin-1-yl]pyrazine-2-carboxamide (D-220)

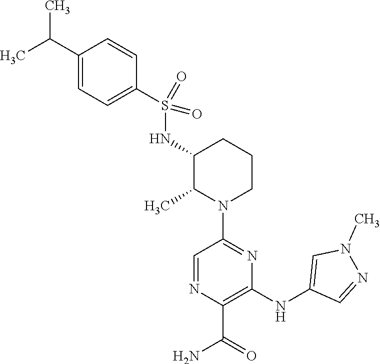 [SMILES: CC(C)c1ccc(S(=O)(=O)N[C@@H]2CCCN(c3cnc(C(N)=O)c(Nc4cnn(C)c4)n3)[C@@H]2C)cc1]

In a similar manner as described in Example 7 (except HCl treatment) 3-[(1-methyl-1H-pyrazol-4-yl)amino]-5-[(2R,3R)-2-methyl-3-[4-(propan-2-yl)benzenesulfonamido]piperidin-1-yl]pyrazine-2-carboxamide (D-220) was prepared using 4-(propan-2-yl)benzene-1-sulfonyl chloride. MS found for C24H32N8O3S as (M+H)⁺ 513.2.

1H NMR (500 MHz, DMSO) δ 10.89 (s, 1H), 7.99-7.88 (m, 2H), 7.75 (d, J=8.37 Hz, 2H), 7.68 (br. s., 1H), 7.53-7.49 (m, 1H), 7.47-7.41 (m, 3H), 7.30 (br. s., 1H), 4.95 (br. s., 1H), 4.10-3.93 (m, 1H), 3.84 (s, 3H), 3.21-3.09 (m, 1H), 3.03-2.87 (m, 2H), 1.80-1.53 (m, 2H), 1.46-1.29 (m, 2H), 1.20 (dd, J=6.93, 1.44 Hz, 6H), 1.15 (d, J=6.86 Hz, 3H).

### Example D-221: Synthesis of 3-[(1-Methyl-1H-pyrazol-4-yl)amino]-5-[(2R,3R)-2-methyl-3-{4-[1-(trifluoromethyl)cyclopropyl]benzamido}piperidin-1-yl]pyrazine-2-carboxamide (D-221)

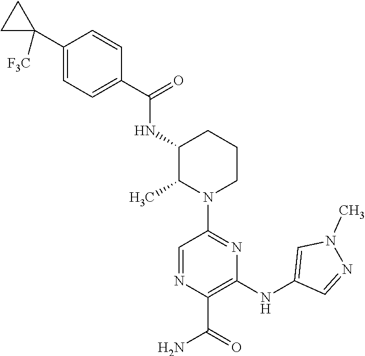 [SMILES: C[C@@H]1[C@H](NC(=O)c2ccc(C3(C(F)(F)F)CC3)cc2)CCCN1c1cnc(C(N)=O)c(Nc2cnn(C)c2)n1]

In a similar manner as described in Example 8, 3-[(1-methyl-1H-pyrazol-4-yl)amino]-5-[(2R,3R)-2-methyl-3-{4-[1-(trifluoromethyl)cyclopropyl]benzamido}piperidin-1-yl]pyrazine-2-carboxamide (D-221) was prepared using 4-[1-(trifluoromethyl)cyclopropyl]benzoic acid. MS found for C26H29F3N8O2 as (M+H)⁺ 543.2.

¹H NMR (500 MHz, DMSO) δ 10.86 (s, 1H), 8.47 (d, J=6.86 Hz, 1H), 8.00 (br. s., 1H), 7.90 (d, J=8.23 Hz, 2H), 7.68 (br. s., 1H), 7.59-7.50 (m, 3H), 7.46 (s, 1H), 7.27 (br. s., 1H), 5.57-5.00 (m, 1H), 4.12 (s, 1H), 4.03-3.98 (m, 1H), 3.75 (s, 3H), 3.07 (t, J=11.94 Hz, 1H), 1.93 (qd, J=12.99, 3.57 Hz, 1H), 1.84 (d, J=13.17 Hz, 1H), 1.70 (d, J=9.61 Hz, 1H), 1.65-1.52 (m, 1H), 1.41-1.33 (m, 2H), 1.16 (br. s., 2H), 1.08 (d, J=6.86 Hz, 3H).

### Example D-222: Synthesis of 3-[(1-Methyl-1H-pyrazol-4-yl)amino]-5-[(3R)-3-[4-(propan-2-yl)benzamido]piperidin-1-yl]pyrazine-2-carboxamide (D-222)

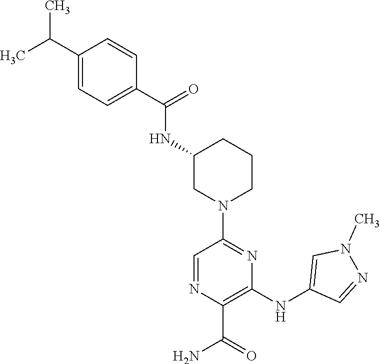 [SMILES: CC(C)c1ccc(C(=O)N[C@@H]2CCCN(c3cnc(C(N)=O)c(Nc4cnn(C)c4)n3)C2)cc1]

In a similar manner as described in Example 8, 3-[(1-methyl-1H-pyrazol-4-yl)amino]-5-[(3R)-3-[4-(propan-2-yl)benzamido]piperidin-1-yl]pyrazine-2-carboxamide (D-222) was prepared using 4-(propan-2-yl)benzoic acid. MS found for C24H30N8O2 as (M+H)⁺ 463.2.

¹H NMR (500 MHz, DMSO) δ 10.86 (s, 1H), 8.33 (d, J=7.14 Hz, 1H), 8.00 (s, 1H), 7.80 (d, J=8.23 Hz, 2H), 7.69 (d, J=2.20 Hz, 1H), 7.60 (s, 1H), 7.49 (s, 1H), 7.34 (d, J=8.23 Hz, 2H), 7.26 (d, J=2.20 Hz, 1H), 4.55 (br. s., 1H), 4.18 (d, J=13.45 Hz, 1H), 4.01-3.87 (m, 1H), 3.76 (s, 3H), 3.19-3.08 (m, 1H), 3.06-2.98 (m, 1H), 2.94 (dt, J=13.72, 6.86 Hz, 1H), 1.98 (dd, J=12.76, 3.98 Hz, 1H), 1.92-1.84 (m, 1H), 1.81-1.69 (m, 1H), 1.65-1.52 (m, 1H), 1.22 (d, J=7.14 Hz, 6H).

### Example D-223: Synthesis of 5-[(2R,3R)-3-(4-tert-butyl-2-fluorobenzamido)-2-methylpiperidin-1-yl]-3-[(1-methyl-1H-pyrazol-4-yl)amino]pyrazine-2-carboxamide (D-223)

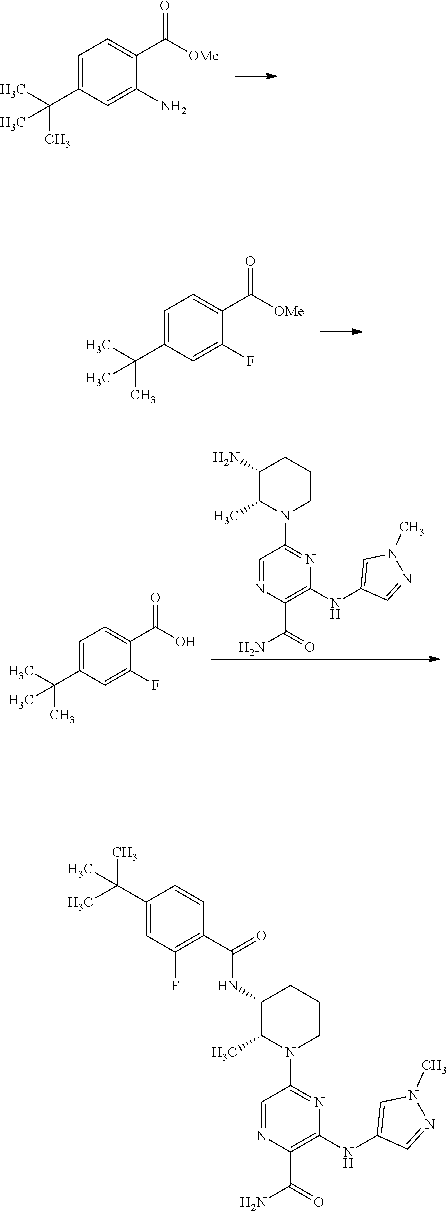 [SMILES: COC(=O)c1ccc(C(C)(C)C)cc1N ; COC(=O)c1ccc(C(C)(C)C)cc1F ; CC(C)(C)c1ccc(C(=O)O)c(F)c1 ; C[C@@H]1[C@H](N)CCCN1c1cnc(C(N)=O)c(Nc2cnn(C)c2)n1 ; C[C@@H]1[C@H](NC(=O)c2ccc(C(C)(C)C)cc2F)CCCN1c1cnc(C(N)=O)c(Nc2cnn(C)c2)n1]

To a solution of BF₃-Et₂O (74 μL, 0.6 mmol) in DME (3 mL) at −10° C. a solution of methyl 2-amino-4-tert-butylbenzoate (100 mg, 0.48 mmol) in DME (3 mL) was added. The reaction mixture was stirred at this temperature for 30 minutes. A solution of t-BuONO (60 μL, 0.5 mmol) in DME (3 mL) was added dropwise. The mixture was stirred at −10/0° C. for 3 h then it was concentrated. The residue was dissolved in chlorobenzene (10 mL) then it was heated to 130° C. and stirred at this temperature for 2 h. The mixture was left to reach room temperature then it was concentrated and purified by silica flash chromatography with 0 to 100% dichloromethane in cyclohexane to give methyl 4-tert-butyl-2-fluorobenzoate (48 mg, 47% yield) as a yellow oil. MS found for C12H15F02 as (M+H)⁺ 211.0

To a solution of 4-tert-butyl-2-fluorobenzoate (48 mg, 0.23 mmol) in MeOH (2 mL) a solution of NaOH (18.4 mg, 0.46 mmol in 0.5 mL of H₂O) was added. The mixture was refluxed for 3 h then it was acidified to pH 3. It was partitioned between DCM and H₂O. The organic phase was dried over Na₂SO₄, filtered and evaporated to dryness to give 4-tert-butyl-2-fluorobenzoic acid (45 mg, quant. yield). MS found for C11H13FO2 as (M+H)⁺ 197.0.

In a similar manner as described in Example 8, 5-[(2R,3R)-3-(4-teroom temperature-butyl-2-fluorobenzamido)-2-methylpiperidin-1-yl]-3-[(1-methyl-1H-pyrazol-4-yl)amino]pyrazine-2-carboxamide (D-223) was prepared using 4-tert-butyl-2-fluorobenzoic acid. MS found for C26H33FN8O2 as (M+H)⁺ 509.2.

¹H NMR (500 MHz, DMSO) δ 10.89 (s, 1H), 8.40 (d, J=6.58 Hz, 1H), 8.03 (s, 1H), 7.69 (br. s., 1H), 7.60-7.45 (m, 3H), 7.36-7.20 (m, 3H), 5.47-5.20 (m, 1H), 4.20-3.94 (m, 2H), 3.77 (s, 3H), 3.16-2.98 (m, 1H), 1.92-1.49 (m, 4H), 1.30 (s, 9H), 1.12 (d, J=7.02 Hz, 3H).

### Example D-224: Synthesis of 5-[(2R,3R)-3-(2,3-dihydro-1,4-benzodioxine-6-amido)-2-methylpiperidin-1-yl]-3-[(1-methyl-1H-pyrazol-4-yl)amino]pyrazine-2-carboxamide (D-224)

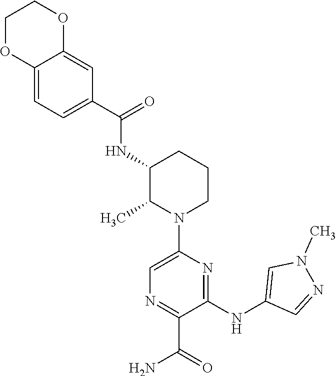 [SMILES: C[C@@H]1[C@H](NC(=O)c2ccc3c(c2)OCCO3)CCCN1c1cnc(C(N)=O)c(Nc2cnn(C)c2)n1]

In a similar manner as described in Example 8, 5-[(2R,3R)-3-(2,3-dihydro-1,4-benzodioxine-6-amido)-2-methylpiperidin-1-yl]-3-[(1-methyl-1H-pyrazol-4-yl)amino]pyrazine-2-carboxamide (D-224) was prepared using 2,3-dihydro-1,4-benzodioxane-6-carboxylic-acid. MS found for C24H28N8O4 as (M+H)⁺ 493.3.

¹H NMR (500 MHz, DMSO) δ 10.86 (s, 1H), 8.26 (d, J=6.80 Hz, 1H), 8.02 (s, 1H), 7.68 (br. s., 1H), 7.56 (s, 1H), 7.50-7.42 (m, 3H), 7.27 (br. s., 1H), 6.94 (d, J=8.33 Hz, 1H), 5.46-5.06 (m, 1H), 4.37-4.24 (m, 4H), 4.20-4.05 (m, 1H), 3.99 (dd, J=11.29, 6.47 Hz, 1H), 3.77 (s, 3H), 3.15-3.01 (m, 1H), 2.04-1.77 (m, 2H), 1.74-1.47 (m, 2H), 1.07 (d, J=7.02 Hz, 3H).

### Example D-225: Synthesis of 5-[(3R)-3-(6-cyclopropyl-1-oxo-1,2-dihydro-2,7-naphthyridin-2-yl)piperidin-1-yl]-3-[(1-methyl-1H-pyrazol-4-yl)amino]pyrazine-2-carboxamide (D-225)

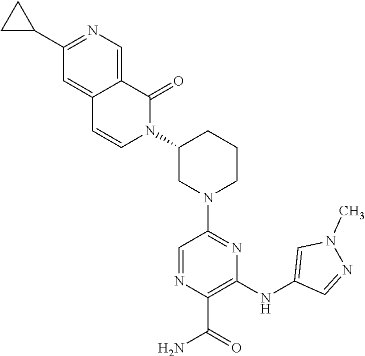 [SMILES: Cn1cc(Nc2nc(N3CCC[C@@H](n4ccc5cc(C6CC6)ncc5c4=O)C3)cnc2C(N)=O)cn1]

In a similar manner as described in Example D-165 5-[(3R)-3-(6-cyclopropyl-1-oxo-1,2-dihydro-2,7-naphthyridin-2-yl)piperidin-1-yl]-3-[(1-methyl-1H-pyrazol-4-yl)amino]pyrazine-2-carboxamide (D-225) was prepared using 5-[(3R)-3-(6-cyclopropyl-1-oxo-1,2-dihydro-2,7-naphthyridin-2-yl)piperidin-1-yl]-3-[(1-methyl-1H-pyrazol-4-yl)amino]pyrazine-2-carbonitrile. MS found for C25H27N9O2 as (M+H)⁺ 486.4.

¹H NMR (500 MHz, DMSO) δ 10.83 (s, 1H), 9.23 (s, 1H), 7.85 (t, J=3.72 Hz, 2H), 7.73 (br. s., 1H), 7.67 (s, 1H), 7.48 (d, J=5.87 Hz, 2H), 7.31 (br. s., 1H), 6.63 (d, J=7.43 Hz, 1H), 4.93-4.80 (m, 1H), 4.56 (d, J=11.74 Hz, 1H), 4.41 (d, J=14.09 Hz, 1H), 3.63 (s, 3H), 3.25-3.37 (m, 1H), 3.09 (t, J=11.93 Hz, 1H), 2.28-2.09 (m, 2H), 2.01-1.90 (m, 2H), 1.70 (d, J=12.52 Hz, 1H), 0.93-1.09 (m, 4H).

### Example D-226: Synthesis of N-[(2R,3R)-1-(5-carbamoyl-6-{[4-(4-methylpiperazin-1-yl)phenyl]amino}pyrazin-2-yl)-2-methylpiperidin-3-yl]-1,3-benzothiazole-5-carboxamide (D-226)

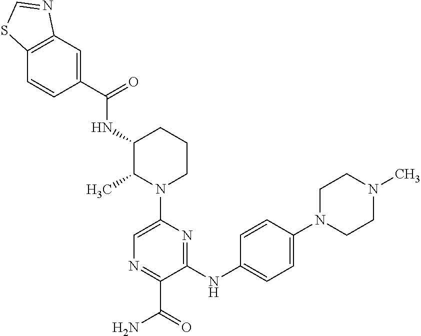 [SMILES: C[C@@H]1[C@H](NC(=O)c2ccc3scnc3c2)CCCN1c1cnc(C(N)=O)c(Nc2ccc(N3CCN(C)CC3)cc2)n1]

In a similar manner as described in Example 8, N-[(2R,3R)-1-(5-carbamoyl-6-{[4-(4-methylpiperazin-1-yl)phenyl]amino}pyrazin-2-yl)-2-methylpiperidin-3-yl]-1,3-benzothiazole-5-carboxamide (D-226) was prepared using benzothiazole-5-carboxylic acid. MS found for C30H35N9O2S as (M+H)⁺ 486.3.

¹H NMR (500 MHz, DMSO) δ 10.96 (s, 1H), 9.52 (s, 1H), 8.70 (d, J=0.98 Hz, 1H), 8.67 (d, J=7.43 Hz, 1H), 8.31 (d, J=8.22 Hz, 1H), 8.07 (dd, J=8.41, 1.17 Hz, 1H), 7.71 (br. s., 1H), 7.61 (s, 1H), 7.46 (d, J=9.00 Hz, 2H), 7.27 (br. s., 1H), 6.80 (d, J=8.61 Hz, 2H), 5.21 (br. s., 1H), 4.26-4.02 (m, 2H), 3.09 (t, J=12.32 Hz, 1H), 2.87 (br. s., 4H), 2.27 (br. s., 4H), 2.16 (s, 3H), 2.07-1.92 (m, 1H), 1.87 (d, J=12.32 Hz, 1H), 1.80-1.69 (m, 1H), 1.68-1.54 (m, 1H), 1.11 (d, J=6.85 Hz, 3H).

### Example D-227: Synthesis of 5-[(2R,3R)-3-(4-methanesulfonylbenzamido)-2-methylpiperidin-1-yl]-3-{[4-(4-methylpiperazin-1-yl)phenyl]amino}pyrazine-2-carboxamide (D-227)

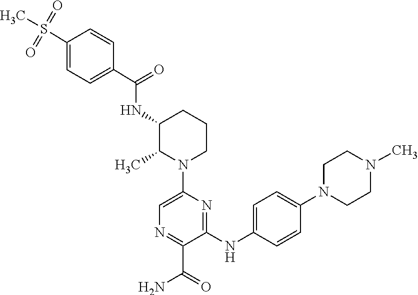

In a similar manner as described in Example 8, 5-[(2R,3R)-3-(4-methanesulfonylbenzamido)-2-methylpiperidin-1-yl]-3-{[4-(4-methylpiperazin-1-yl)phenyl]amino}pyrazine-2-carboxamide (D-227) was prepared using 4-methanesulfonylbenzoic acid. MS found for C30H38N8O4S as (M+H)⁺ 607.3.

¹H NMR (500 MHz, DMSO) δ 10.95 (s, 1H), 8.73 (d, J=7.43 Hz, 1H), 8.21-8.13 (m, 2H), 8.10-8.03 (m, 2H), 7.71 (br. s., 1H), 7.60 (s, 1H), 7.45 (d, J=9.00 Hz, 2H), 7.28 (br. s., 1H), 6.80 (d, J=8.61 Hz, 2H), 5.31-4.96 (m, 1H), 4.25-3.98 (m, 2H), 3.29 (s, 3H), 3.07 (t, J=11.54 Hz, 1H), 2.92 (br. s., 4H), 2.36 (br. s., 4H), 2.19 (s, 3H), 2.07-1.80 (m, 2H), 1.77-1.52 (m, 2H), 1.09 (d, J=7.04 Hz, 3H).

### Example D-228: Synthesis of 3-[(1-Methyl-1H-pyrazol-4-yl)amino]-5-[(2R,3R)-2-methyl-3-[4-(3-methyloxetan-3-yl)benzamido]piperidin-1-yl]pyrazine-2-carboxamide (D-228)

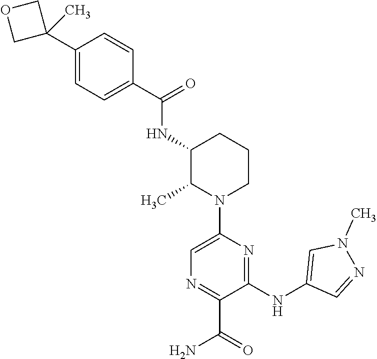 [SMILES: C[C@@H]1[C@H](NC(=O)c2ccc(C3(C)COC3)cc2)CCCN1c1cnc(C(N)=O)c(Nc2cnn(C)c2)n1]

In a similar manner as described in Example 8, 3-[(1-methyl-1H-pyrazol-4-yl)amino]-5-[(2R,3R)-2-methyl-3-[4-(3-methyloxetan-3-yl)benzamido]piperidin-1-yl]pyrazine-2-carboxamide (D-228) was prepared using 4-methanesulfonylbenzoic acid. MS found for C26H32N8O3 as (M+H)⁺ 505.3

¹H NMR (500 MHz, DMSO) δ 10.85 (s, 1H), 8.40 (d, J=6.80 Hz, 1H), 8.01 (br. s., 1H), 7.90 (d, J=8.33 Hz, 2H), 7.67 (br. s., 1H), 7.55 (s, 1H), 7.46 (s, 1H), 7.34 (d, J=8.33 Hz, 2H), 7.26 (br. s., 1H), 5.48-5.10 (m, 1H), 4.81 (d, J=5.48 Hz, 2H), 4.56 (d, J=5.70 Hz, 2H), 4.21-3.93 (m, 2H), 3.76 (s, 3H), 3.07 (t, J=12.17 Hz, 1H), 2.10-1.80 (m, 2H), 1.75-1.49 (m, 5H), 1.08 (d, J=6.80 Hz, 3H).

### Example D-229: Synthesis of 5-[(2R,3R)-3-(5-tert-butylthiophene-2-amido)-2-methylpiperidin-1-yl]-3-[(1-methyl-1H-pyrazol-4-yl)amino]pyrazine-2-carboxamide (D-229)

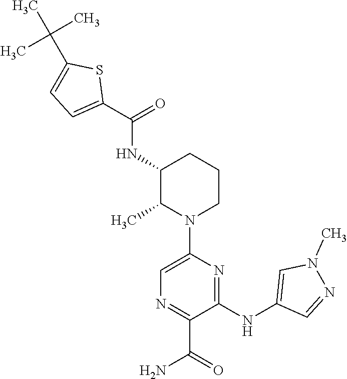 [SMILES: C[C@@H]1[C@H](NC(=O)c2ccc(C(C)(C)C)s2)CCCN1c1cnc(C(N)=O)c(Nc2cnn(C)c2)n1]

In a similar manner as described in Example 7 (except HCl treatment) 5-[(2R,3R)-3-(5-tert-butylthiophene-2-amido)-2-methylpiperidin-1-yl]-3-[(1-methyl-1H-pyrazol-4-yl)amino]pyrazine-2-carboxamide (D-229) was prepared using 5-tert-butyl-thiophene-2-carbonyl chloride. MS found for C24H32N8O2S as (M+H)⁺ 497.3.

¹H NMR (500 MHz, DMSO) δ 10.87 (s, 1H), 8.33 (d, J=6.80 Hz, 1H), 8.02 (s, 1H), 7.74 (d, J=3.95 Hz, 1H), 7.68 (br. s., 1H), 7.55 (s, 1H), 7.46 (s, 1H), 7.27 (br. s., 1H), 6.95 (d, J=3.73 Hz, 1H), 5.54-5.06 (m, 1H), 4.25-4.04 (m, 1H), 4.02-3.90 (m, 1H), 3.82 (s, 3H), 3.08 (t, J=12.17 Hz, 1H), 2.01-1.80 (m, 2H), 1.78-1.52 (m, 2H), 1.35 (s, 9H), 1.07 (d, J=6.80 Hz, 3H).

### Example D-230: Synthesis of 3-[(1-methyl-1H-pyrazol-4-yl)amino]-5-[(2R,3R)-2-methyl-3-[4-(pentafluoro-λ⁶-sulfanyl)benzamido]piperidin-1-yl]pyrazine-2-carboxamide (D-230)

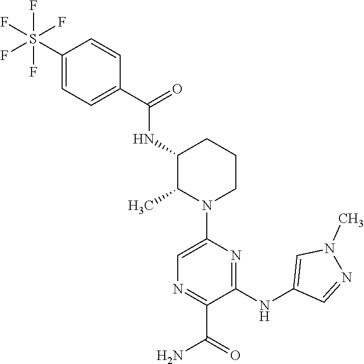 [SMILES: C[C@@H]1[C@H](NC(=O)c2ccc(S(F)(F)(F)(F)F)cc2)CCCN1c1cnc(C(N)=O)c(Nc2cnn(C)c2)n1]

In a similar manner as described in Example 8, 3-[(1-methyl-1H-pyrazol-4-yl)amino]-5-[(2R,3R)-2-methyl-3-[4-(pentafluoro-λ⁶-sulfanyl)benzamido]piperidin-1-yl]pyrazine-2-carboxamide (D-230) was prepared using 4-(pentafluoro-X⁶-sulfanyl)benzoic acid. MS found for C22H25F5N8O2S as (M+H)⁺ 561.1.

¹H NMR (500 MHz, DMSO) δ 10.87 (s, 1H), 8.74 (d, J=7.04 Hz, 1H), 8.16-8.03 (m, 4H), 8.02-7.98 (m, 1H), 7.74-7.66 (m, 1H), 7.57 (s, 1H), 7.48 (s, 1H), 7.28 (br. s., 1H), 5.45-5.16 (m, 1H), 4.05 (m, J=12.50, 7.00 Hz, 2H), 3.76 (s, 3H), 3.09 (t, J=11.74 Hz, 1H), 2.03-1.49 (m, 4H), 1.11 (d, J=6.65 Hz, 3H).

### Example D-231: Synthesis of 5-[(2R,3R)-3-(4-cyclopropyl-3-methoxybenzamido)-2-methylpiperidin-1-yl]-3-[(1-methyl-1H-pyrazol-4-yl)amino]pyrazine-2-carboxamide (D-231)

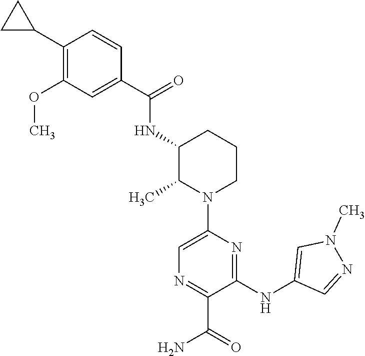 [SMILES: COc1cc(C(=O)N[C@@H]2CCCN(c3cnc(C(N)=O)c(Nc4cnn(C)c4)n3)[C@@H]2C)ccc1C1CC1]

In a similar manner as described in Example 8, 5-[(2R,3R)-3-(4-cyclopropyl-3-methoxybenzamido)-2-methylpiperidin-1-yl]-3-[(1-methyl-1H-pyrazol-4-yl)amino]pyrazine-2-carboxamide (D-231) was prepared using 4-cyclopropyl-3-methoxybenzoic acid. MS found for C26H32N8O₃ as (M+H)⁺ 505.3.

¹H NMR (500 MHz, DMSO) δ 10.88 (s, 1H), 8.36 (d, J=6.59 Hz, 1H), 8.05 (br. s., 1H), 7.69 (br. s., 1H), 7.57 (s, 1H), 7.50-7.38 (m, 3H), 7.28 (br. s., 1H), 6.91 (d, J=7.96 Hz, 1H), 5.65-4.92 (m, 1H), 4.31-3.97 (m, 2H), 3.88 (s, 3H), 3.79 (s, 3H), 3.17-3.01 (m, 1H), 2.23-2.10 (m, 1H), 2.05-1.81 (m, 2H), 1.79-1.54 (m, 2H), 1.09 (d, J=6.86 Hz, 3H), 1.00-0.89 (m, 2H), 0.59-0.77 (m, 2H).

### Example D-232: Synthesis of 3-[(1-methyl-1H-pyrazol-4-yl)amino]-5-[(2R,3R)-2-methyl-3-(4-methylbenzamido)piperidin-1-yl]pyrazine-2-carboxamide (D-232)

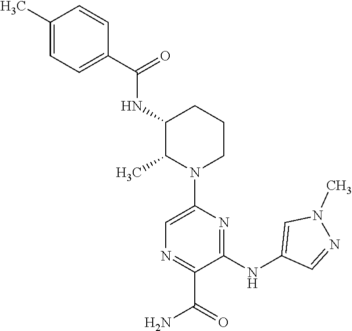 [SMILES: Cc1ccc(C(=O)N[C@@H]2CCCN(c3cnc(C(N)=O)c(Nc4cnn(C)c4)n3)[C@@H]2C)cc1]

In a similar manner as described in Example 8, 3-[(1-methyl-1H-pyrazol-4-yl)amino]-5-[(2R,3R)-2-methyl-3-(4-methylbenzamido)piperidin-1-yl]pyrazine-2-carboxamide (D-232) was prepared using p-toluic acid. MS found for C23H28N8O2 as (M+H)⁺ 449.3.

¹H NMR (500 MHz, DMSO) δ 10.84 (s, 1H), 8.34 (d, J=6.85 Hz, 1H), 8.01 (s, 1H), 7.82 (d, J=8.22 Hz, 2H), 7.67 (br. s., 1H), 7.55 (s, 1H), 7.45 (s, 1H), 7.33-7.20 (m, 3H), 5.29 (br. s., 1H), 4.24-3.94 (m, 2H), 3.73 (s, 3H), 3.07 (t, J=11.64 Hz, 1H), 2.35 (s, 3H), 2.05-1.79 (m, 2H), 1.76-1.50 (m, 2H), 1.07 (d, J=6.85 Hz, 3H).

### Example D-233: Synthesis of 5-[(2R,3R)-2-methyl-3-{4-[(trifluoromethyl)sulfanyl]benzamido}piperidin-1-yl]-3-{[4-(4-methylpiperazin-1-yl)phenyl]amino}pyrazine-2-carboxamide (D-233)

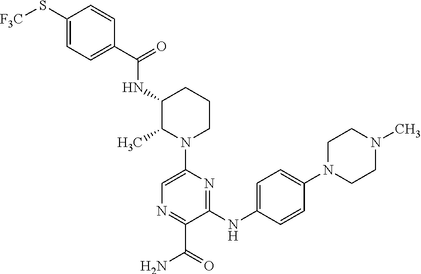 [SMILES: C[C@@H]1[C@H](NC(=O)c2ccc(SC(F)(F)F)cc2)CCCN1c1cnc(C(N)=O)c(Nc2ccc(N3CCN(C)CC3)cc2)n1]

In a similar manner as described in Example 8, 5-[(2R,3R)-2-methyl-3-{4-[(trifluoromethyl)sulfanyl]benzamido}piperidin-1-yl]-3-{[4-(4-methylpiperazin-1-yl)phenyl]amino}pyrazine-2-carboxamide (D-233) was prepared using 4-[(trifluoromethyl)sulfanyl]benzoic acid. MS found for C30H35F3N8O2S as (M+H)⁺ 629.0.

¹H NMR (500 MHz, DMSO) δ 10.96 (s, 1H), 8.66 (d, J=7.04 Hz, 1H), 8.05 (d, J=8.22 Hz, 2H), 7.86 (d, J=8.22 Hz, 2H), 7.71 (br. s., 1H), 7.60 (s, 1H), 7.45 (d, J=9.00 Hz, 2H), 7.28 (br. s., 1H), 6.81 (d, J=9.00 Hz, 2H), 5.32-4.93 (m, 1H), 4.28-4.01 (m, 2H), 3.15-2.99 (m, 2H), 2.97-2.84 (m, 4H), 2.36 (br. s., 4H), 2.19 (s, 3H), 2.01-1.79 (m, 2H), 1.77-1.57 (m, 2H), 1.09 (d, J=7.04 Hz, 2H).

### Example D-234: Synthesis of N-[(2R,3R)-1-(5-carbamoyl-6-{[4-(4-methylpiperazin-1-yl)phenyl]amino}pyrazin-2-yl)-2-methylpiperidin-3-yl]-1-methyl-1H-indazole-5-carboxamide (D-234)

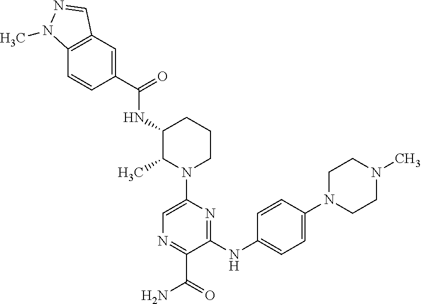

In a similar manner as described in Example 8, N-[(2R,3R)-1-(5-carbamoyl-6-{[4-(4-methylpiperazin-1-yl)phenyl]amino}pyrazin-2-yl)-2-methylpiperidin-3-yl]-1-methyl-1H-indazole-5-carboxamide (D-234) was prepared using 1-methyl-1H-indazole-5-carboxylic acid. MS found for C31H38N10O2 as (M+H)⁺ 583.3.

¹H NMR (500 MHz, DMSO) δ 10.92 (br. s., 1H), 8.47 (d, J=7.41 Hz, 1H), 8.42-8.37 (m, 1H), 8.20 (s, 1H), 7.98 (dd, J=8.78, 1.10 Hz, 1H), 7.74-7.67 (m, 2H), 7.59 (s, 1H), 7.43 (d, J=9.06 Hz, 2H), 7.26 (br. s., 1H), 6.75 (d, J=7.68 Hz, 2H), 5.16 (br. s., 1H), 4.26-3.94 (m, 5H), 3.12-2.99 (m, 1H), 2.90-2.67 (m, 4H), 2.30-2.07 (m, 7H), 2.02-1.51 (m, 4H), 1.09 (d, J=6.59 Hz, 3H).

### Example D-235: Synthesis of 5-[(2R,3R)-3-[4-(1-hydroxycyclopropyl)benzamido]-2-methylpiperidin-1-yl]-3-[(1-methyl-1H-pyrazol-4-yl)amino]pyrazine-2-carboxamide (D-235)

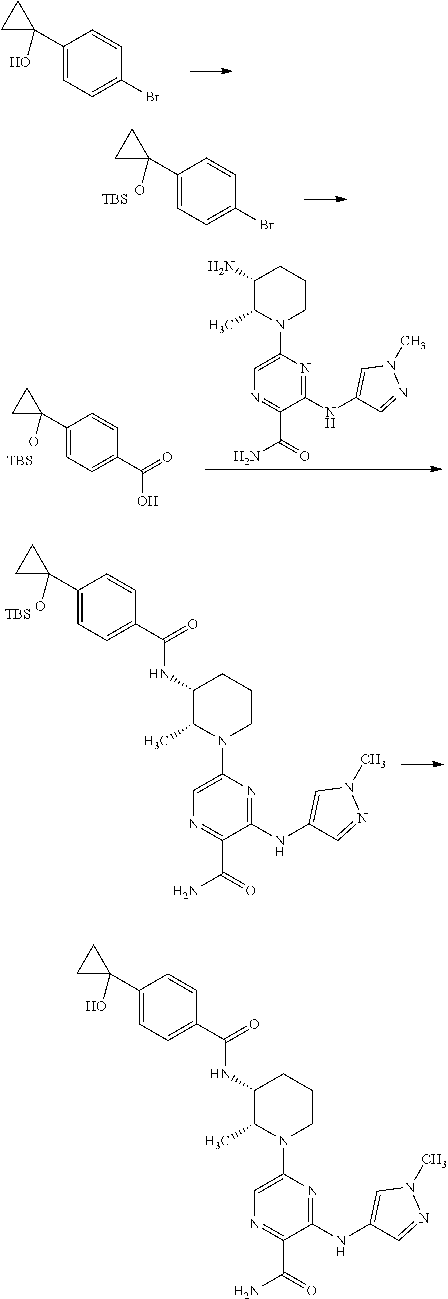

To a solution of 1-(4-bromophenyl)cyclopropan-1-ol (250 mg, 1.17 mmol) in DCM (5 mL) imidazole (96 mg, 1.404 mmol) and TBDMSCl (195 mg, 1.29 mmol) were added. The mixture was stirred at room temperature for 2 h then it was partitioned between H₂O and DCM. The combined organic phases were dried over Na₂SO₄, filtered and evaporated to dryness to give [1-(4-bromophenyl)cyclopropoxy](tert-butyl)dimethylsilane (353 mg, 92% yield).

¹H NMR (400 MHz, DMSO) δ 7.50 (d, J=8.53 Hz, 2H) 7.25 (d, J=8.78 Hz, 2H) 1.14 (d, J=2.26 Hz, 2H) 1.05-0.98 (m, 2H) 0.85 (s, 9H) −0.02 (s, 6H).

To a solution of [1-(4-bromophenyl)cyclopropoxy](tert-butyl)dimethylsilane (353 mg, 1.08 mmol) in THF (5 mL) at −78° C. n-BuLi (0.5 mL, 1.29 mmol) was added. The mixture was stirred at this temperature for 20 minutes then dry ice was added. The mixture was left to reach about −10° C. in 2 h then it was quenched with H₂O. It was partitioned between ethyl acetate and H2O. The combined organic layers were dried over Na₂SO₄, filtered and concentrated. The obtained crude was purified by silica flash chromatography with 20 to 100% ethyl acetate in cyclohexane to give 4-{1-[(tert-butyldimethylsilyl)oxy]cyclopropyl}benzoic acid (22 mg, 7% yield). MS found for C16H24O3Si as (M+H)⁺ 293.22

In a similar manner as described in Example 8, 5-[(2R,3R)-3-(4-{1-[(tert-butyldimethylsilyl)oxy]cyclopropyl}benzamido)-2-methylpiperidin-1-yl]-3-[(1-methyl-1H-pyrazol-4-yl)amino]pyrazine-2-carboxamide, was prepared using 4-{1-[(tert-butyldimethylsilyl)oxy]cyclopropyl}benzoic acid (41 mg, 90% yield). MS found for C31H44N8O3Si as (M+H)⁺ 605.1.

To a solution of 5-[(2R,3R)-3-(4-{1-[(tert-butyldimethylsilyl)oxy]cyclopropyl}benzamido)-2-methylpiperidin-1-yl]-3-[(1-methyl-1H-pyrazol-4-yl)amino]pyrazine-2-carboxamide (41 mg, 0.068 mmol) in THF (3 mL) 1M TBAF (70 μL, 0.068 mmol) was added. The mixture was stirred at room temperature for 2 h then it was partitioned between ethyl acetate and brine. The combined organic phases were dried over Na₂SO₄, filtered and concentrated. The obtained crude was purified by C18 flash chromatography with 0 to 100% acetonitrile in water+0.1% HCOOH to give 5-[(2R,3R)-3-[4-(1-hydroxycyclopropyl)benzamido]-2-methylpiperidin-1-yl]-3-[(1-methyl-1H-pyrazol-4-yl)amino]pyrazine-2-carboxamide (20 mg, 60% yield) as a yellow solid (D-235). MS found for C25H30N8O3 as (M+H)⁺ 491.0.

¹H NMR (500 MHz, DMSO) δ 10.86 (s, 1H), 8.37 (d, J=6.80 Hz, 1H), 8.03 (br. s., 1H), 7.86 (d, J=8.55 Hz, 2H), 7.69 (br. s., 1H), 7.57 (s, 1H), 7.47 (s, 1H), 7.32 (d, J=8.33 Hz, 2H), 7.29-7.25 (m, 1H), 6.04 (s, 1H), 5.52-5.07 (m, 1H), 4.03 (dd, J=11.84, 6.80 Hz, 2H), 3.76 (s, 3H), 3.09 (t, J=12.06 Hz, 1H), 2.09-1.51 (m, 4H), 1.17 (d, J=2.19 Hz, 2H), 1.09 (d, J=6.80 Hz, 3H), 1.02 (d, J=2.41 Hz, 2H).

### Example D-236: Synthesis of 5-[(2S,5R)-5-[4-(2-hydroxypropan-2-yl)benzamido]-2-methylpiperidin-1-yl]-3-[(1-methyl-1H-pyrazol-4-yl)amino]pyrazine-2-carboxamide. (D-236)

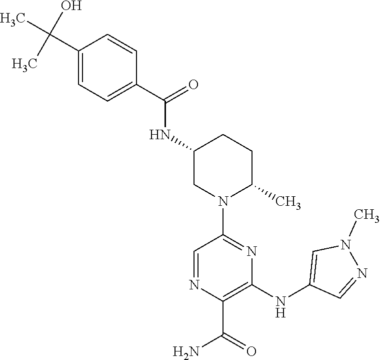 [SMILES: C[C@H]1CC[C@@H](NC(=O)c2ccc(C(C)(C)O)cc2)CN1c1cnc(C(N)=O)c(Nc2cnn(C)c2)n1]

In a similar manner as described in Example D-181, 5-[(2S,5R)-5-[4-(2-hydroxypropan-2-yl)benzamido]-2-methylpiperidin-1-yl]-3-[(1-methyl-1H-pyrazol-4-yl)amino]pyrazine-2-carboxamide (D-236) was prepared starting from tert-butyl N-[(3R,6S)-6-methylpiperidin-3-yl]carbamate. MS found for C25H32N8O3 as (M+H)⁺ 493.1.

¹H NMR (500 MHz, DMSO) δ 10.86 (s, 1H), 8.37 (d, J=7.43 Hz, 1H), 7.97 (s, 1H), 7.85 (d, J=8.61 Hz, 2H), 7.69 (br. s., 1H), 7.62-7.52 (m, 3H), 7.49 (s, 1H), 7.28 (br. s., 1H), 5.13 (s, 1H), 4.85-4.47 (m, 2H), 3.98-3.83 (m, 1H), 3.75 (s, 3H), 3.00-2.76 (m, 1H), 1.97-1.68 (m, 4H), 1.44 (s, 6H), 1.26 (d, J=6.65 Hz, 3H).

### Example D-237: Synthesis of N-[(2R,3R)-1-{5-carbamoyl-6-[(1-methyl-1H-pyrazol-4-yl)amino]pyrazin-2-yl}-2-methylpiperidin-3-yl]-1,3-benzothiazole-5-carboxamide. (D-237)

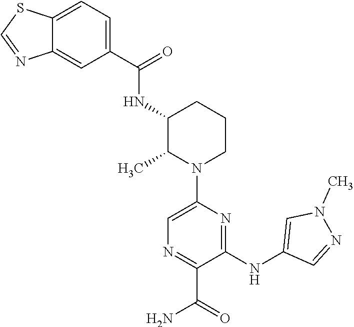 [SMILES: C[C@@H]1[C@H](NC(=O)c2ccc3scnc3c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1,3-benzothiazole-5-carboxamide (D-237) was prepared using benzothiazole-5-carboxylic acid. MS found for C23H25N9O2S as (M+H)⁺ 492.0.

¹H NMR (500 MHz, DMSO) δ 10.88 (s, 1H), 9.51 (s, 1H), 8.70 (d, J=1.10 Hz, 1H), 8.67 (d, J=6.86 Hz, 1H), 8.30 (d, J=8.51 Hz, 1H), 8.04 (m, J=8.20, 1.40 Hz, 2H), 7.70 (br. s., 1H), 7.59 (s, 1H), 7.48 (s, 1H), 5.35 (br.s., 1H), 4.30-4.06 (m, 2H), 3.76 (s, 3H), 3.11 (t, J=12.35 Hz, 1H), 2.13-1.53 (m, 4H), 1.14 (d, J=6.86 Hz, 3H).

### Example D-238: Synthesis of N-[(2R,3R)-1-(5-carbamoyl-6-[(1-methyl-1H-pyrazol-4-yl)amino]pyrazin-2-yl)-2-methylpiperidin-3-yl]-2-methyl-1,3-benzoxazole-5-carboxamide (D-238)

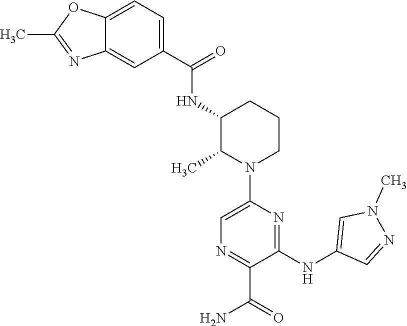 [SMILES: Cc1nc2cc(C(=O)N[C@@H]3CCCN(c4cnc(C(N)=O)c(Nc5cnn(C)c5)n4)[C@@H]3C)ccc2o1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-2-methyl-1,3-benzoxazole-5-carboxamide (D-238) was prepared using 2-methyl-1,3-benzoxazole-5-carboxylic acid. MS found for C24H27N9O3 as (M+H)⁺ 490.3.

¹H NMR (500 MHz, DMSO) δ 10.87 (s, 1H), 8.54 (d, J=6.72 Hz, 1H), 8.26 (d, J=0.82 Hz, 1H), 8.03 (br. s., 1H), 7.96 (dd, J=8.58, 1.44 Hz, 1H), 7.77 (d, J=8.51 Hz, 1H), 7.77 (d, J=8.51 Hz, 1H), 7.70 (br. s., 1H), 7.58 (s, 1H), 7.48 (s, 1H), 7.29 (br. s., 1H), 5.32 (br. s., 1H), 4.25-3.98 (m, 2H), 3.75 (s, 3H), 3.10 (t, J=12.28 Hz, 1H), 2.65 (s, 3H), 2.08-1.92 (m, 1H), 1.88 (d, J=13.31 Hz, 1H), 1.74 (d, J=10.29 Hz, 1H), 1.68-1.55 (m, 1H), 1.12 (d, J=6.86 Hz, 3H).

### Example D-239: Synthesis of 5-[(2R,3R)-3-(2-tert-butyl-1,3-thiazole-5-amido)-2-methylpiperidin-1-yl]-3-[(1-methyl-1H-pyrazol-4-yl)amino]pyrazine-2-carboxamide (D-239)

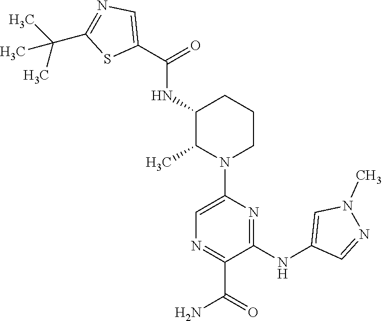 [SMILES: C[C@@H]1[C@H](NC(=O)c2cnc(C(C)(C)C)s2)CCCN1c1cnc(C(N)=O)c(Nc2cnn(C)c2)n1]

In a similar manner as described in Example 8, 5-[(2R,3R)-3-(2-tert-butyl-1,3-thiazole-5-amido)-2-methylpiperidin-1-yl]-3-[(1-methyl-1H-pyrazol-4-yl)amino]pyrazine-2-carboxamide (D-239) was prepared using 2-tert-butyl-1,3-thiazole-5-carboxylic acid. MS found for C23H31N9O2S as (M+H)⁺ 498.0.

¹H NMR (500 MHz, DMSO) δ 10.88 (s, 1H), 8.59 (d, J=6.86 Hz, 1H), 8.38 (s, 1H), 8.00 (br. s., 1H), 7.69 (br. s., 1H), 7.56 (s, 1H), 7.46 (s, 1H), 7.29 (br. s., 1H), 5.29 (br. s., 1H), 4.22-3.90 (m, 2H), 3.80 (s, 3H), 3.09 (t, J=12.35 Hz, 1H), 1.98-1.53 (m, 4H), 1.40 (s, 9H), 1.08 (d, J=6.86 Hz, 3H).

### Example D-240: Synthesis of 5-[(2R,3R)-3-[4-(1,1,1,3,3,3-hexafluoro-2-hydroxypropan-2-yl)benzamido]-2-methylpiperidin-1-yl]-3-[(1-methyl-1H-pyrazol-4-yl)amino]pyrazine-2-carboxamide (D-240)

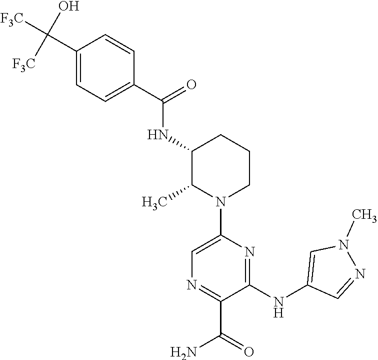 [SMILES: C[C@@H]1[C@H](NC(=O)c2ccc(C(O)(C(F)(F)F)C(F)(F)F)cc2)CCCN1c1cnc(C(N)=O)c(Nc2cnn(C)c2)n1]

In a similar manner as described in Example 8, 5-[(2R,3R)-3-[4-(1,1,1,3,3,3-hexafluoro-2-hydroxypropan-2-yl)benzamido]-2-methylpiperidin-1-yl]-3-[(1-methyl-1H-pyrazol-4-yl)amino]pyrazine-2-carboxamide (D-240) was prepared using 4-(1,1,1,3,3,3-hexafluoro-2-hydroxypropan-2-yl)benzoic acid. MS found for C25H26F6N8O3 as (M+H)⁺ 601.3

¹H NMR (500 MHz, DMSO) δ 10.87 (s, 1H), 8.91 (br. s., 1H), 8.59 (d, J=6.69 Hz, 1H), 8.07-7.96 (m, 3H), 7.81 (d, J=8.22 Hz, 2H), 7.69 (br. s., 1H), 7.57 (s, 1H), 7.48 (s, 1H), 7.28 (br. s., 1H), 5.31 (br. s., 1H), 4.39-3.95 (m, 2H), 3.76 (s, 3H), 3.16-3.02 (m, 1H), 2.06-1.80 (m, 2H), 1.79-1.50 (m, 2H), 1.11 (d, J=6.90 Hz, 3H).

### Example D-241: Synthesis of 3-[(1-methyl-1H-pyrazol-4-yl)amino]-5-[(2R,3R)-2-methyl-3-{4-[(trifluoromethyl)sulfanyl]benzamido}piperidin-1-yl]pyrazine-2-carboxamide (D-241)

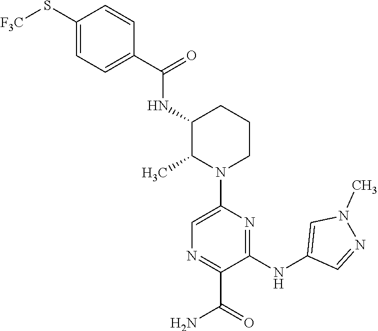 [SMILES: C[C@@H]1[C@H](NC(=O)c2ccc(SC(F)(F)F)cc2)CCCN1c1cnc(C(N)=O)c(Nc2cnn(C)c2)n1]

In a similar manner as described in Example 8, 3-[(1-methyl-1H-pyrazol-4-yl)amino]-5-[(2R,3R)-2-methyl-3-{4-[(trifluoromethyl)sulfanyl]benzamido}piperidin-1-yl]pyrazine-2-carboxamide (D-241) was prepared using 4-[(trifluoromethyl)sulfanyl]benzoic acid. MS found for C23H25F3N8O2S as (M+H)⁺ 535.1.

¹H NMR (500 MHz, DMSO) δ 10.87 (s, 1H), 8.67 (d, J=6.36 Hz, 1H), 8.10-7.95 (m, 3H), 7.86 (d, J=8.31 Hz, 2H), 7.70 (br. s., 1H), 7.57 (s, 1H), 7.48 (s, 1H), 7.29 (br. s., 1H), 5.52-4.96 (m, 1H), 4.31-3.93 (m, 2H), 3.75 (s, 3H), 3.18-3.01 (m, 1H), 2.07-1.51 (m, 4H), 1.11 (d, J=6.85 Hz, 3H).

### Example D-242: Synthesis of N-[(2R,3R)-1-{5-carbamoyl-6-[(1-methyl-1H-pyrazol-4-yl)amino]pyrazin-2-yl}-2-methylpiperidin-3-yl]-2-methyl-1,3-benzothiazole-5-carboxamide (D-242)

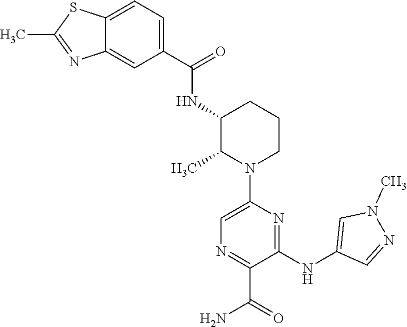 [SMILES: Cc1nc2cc(C(=O)N[C@@H]3CCCN(c4cnc(C(N)=O)c(Nc5cnn(C)c5)n4)[C@@H]3C)ccc2s1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-2-methyl-1,3-benzothiazole-5-carboxamide (D-242) was prepared using 2-methyl-1,3-benzothiazole-5-carboxylic acid. MS found for C24H27N9O2S as (M+H)⁺ 506.2.

¹H NMR (500 MHz, DMSO) δ 10.87 (s, 1H), 8.61 (d, J=6.85 Hz, 1H), 8.50 (d, J=0.98 Hz, 1H), 8.15 (d, J=8.31 Hz, 1H), 8.04 (br. s., 1H), 7.95 (dd, J=8.31, 1.47 Hz, 1H), 7.70 (br. s., 1H), 7.58 (s, 1H), 7.48 (s, 1H), 7.28 (br. s., 1H), 5.55-5.00 (m, 1H), 4.30-3.99 (m, 2H), 3.76 (s, 3H), 3.11 (t, J=12.23 Hz, 1H), 2.84 (s, 3H), 2.08-1.52 (m, 4H), 1.13 (d, J=6.85 Hz, 3H).

### Example D-243: Synthesis of N-[(2R,3R)-1-{5-carbamoyl-6-[(1-methyl-1H-pyrazol-4-yl)amino]pyrazin-2-yl}-2-methylpiperidin-3-yl]-1,3-benzothiazole-6-carboxamide (D-243)

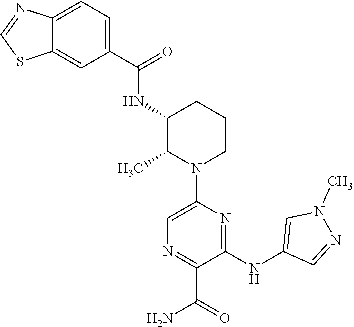 [SMILES: C[C@@H]1[C@H](NC(=O)c2ccc3ncsc3c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1,3-benzothiazole-6-carboxamide (D-243) was prepared using benzothiazole-6-carboxylic acid. MS found for C23H25N9O2S as (M+H)⁺ 492.2.

¹H NMR (500 MHz, DMSO) δ 10.87 (s, 1H), 9.55 (s, 1H), 8.74 (s, 1H), 8.62 (d, J=6.86 Hz, 1H), 8.19 (d, J=8.51 Hz, 1H), 8.08 (dd, J=8.65, 1.51 Hz, 1H), 8.03 (br. s., 1H), 7.70 (br. s., 1H), 7.58 (s, 1H), 7.48 (s, 1H), 7.29 (br. s., 1H), 5.32 (br. s., 1H), 4.24-4.05 (m, 2H), 3.75 (s, 3H), 3.16-3.04 (m, 1H), 2.07-1.56 (m, 4H), 1.14 (d, J=6.59 Hz, 3H).

### Example D-244: Synthesis of 5-[(2R,3R)-3-(3-tert-butyl-1H-pyrazole-5-amido)-2-methylpiperidin-1-yl]-3-[(1-methyl-1H-pyrazol-4-yl)amino]pyrazine-2-carboxamide (D-244)

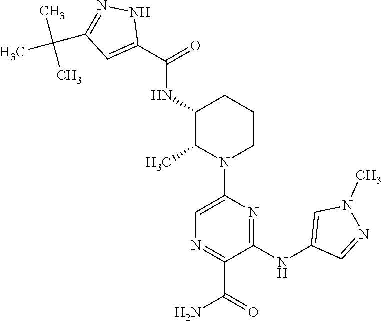 [SMILES: C[C@@H]1[C@H](NC(=O)c2cc(C(C)(C)C)n[nH]2)CCCN1c1cnc(C(N)=O)c(Nc2cnn(C)c2)n1]

In a similar manner as described in Example 8, 5-[(2R,3R)-3-(3-tert-butyl-1H-pyrazole-5-amido)-2-methylpiperidin-1-yl]-3-[(1-methyl-1H-pyrazol-4-yl)amino]pyrazine-2-carboxamide (D-244) was prepared using 3-tert-butyl-1H-pyrazole-5-carboxylic acid. MS found for C23H32N10O2 as (M+H)⁺ 481.2

¹H NMR (500 MHz, DMSO) δ 13.33-12.85 (m, 1H), 10.85 (s, 1H), 8.37-8.01 (m, 1H), 7.99-7.91 (m, 1H), 7.69 (br. s., 1H), 7.55 (s, 1H), 7.46 (s, 1H), 7.27 (br. s., 1H), 6.98-6.40 (m, 1H), 5.51-4.93 (m, 1H), 4.25-3.95 (m, 2H), 3.81 (s, 3H), 3.06 (t, J=12.28 Hz, 1H), 2.07-1.53 (m, 4H), 1.30 (s, 9H), 1.07 (d, J=6.58 Hz, 3H).

### Example D-245: Synthesis of 5-[(2R,3R)-3-(4-cyclopropyl-3-hydroxybenzamido)-2-methylpiperidin-1-yl]-3-[(1-methyl-1H-pyrazol-4-yl)amino]pyrazine-2-carboxamide (D-245)

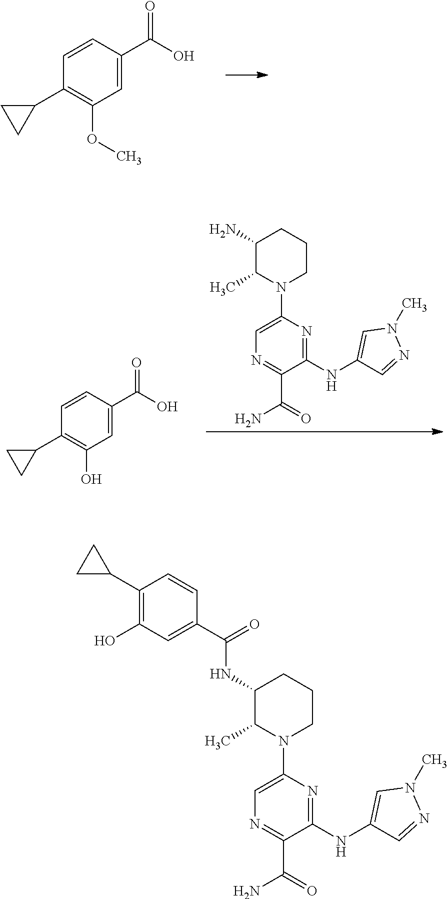 [SMILES: COc1cc(C(=O)O)ccc1C1CC1 ; O=C(O)c1ccc(C2CC2)c(O)c1 ; C[C@@H]1[C@H](N)CCCN1c1cnc(C(N)=O)c(Nc2cnn(C)c2)n1 ; C[C@@H]1[C@H](NC(=O)c2ccc(C3CC3)c(O)c2)CCCN1c1cnc(C(N)=O)c(Nc2cnn(C)c2)n1]

To a solution of 4-cyclopropyl-3-methoxybenzoic acid (83 mg, 0.43 mmol) in DCM (5 mL) at −15° C. 1M BBr₃ (0.6 mL, 0.645 mmol) was added. The mixture was stirred at this temperature for 45 minutes then it was quenched with H₂O. It was partitioned between DCM and brine. The combined organic phases were dried over Na₂SO₄, filtered and concentrated. The crude was purified by C18 flash chromatography with 0 to 100% acetonitrile in water 0.1% HCOOH to give 4-cyclopropyl-3-hydroxybenzoic acid (20 mg, 26% yield) as a white solid. MS found for C10H10O3 as (M+H)⁺ 179.0.

In a similar manner as described in Example 8, 5-[(2R,3R)-3-(4-cyclopropyl-3-hydroxybenzamido)-2-methylpiperidin-1-yl]-3-[(1-methyl-1H-pyrazol-4-yl)amino]pyrazine-2-carboxamide (D-245) was prepared using 4-cyclopropyl-3-hydroxybenzoic acid. MS found for C25H30N8O3 as (M+H)⁺ 491.3.

¹H NMR (500 MHz, DMSO) δ 10.86 (s, 1H), 9.59 (s, 1H), 8.27 (d, J=6.36 Hz, 1H), 8.03 (br. s., 1H), 7.69 (br. s., 1H), 7.56 (s, 1H), 7.50-7.42 (m, 1H), 7.36-7.24 (m, 3H), 6.81 (d, J=7.83 Hz, 1H), 5.31 (br. s., 1H), 4.21-3.90 (m, 2H), 3.77 (s, 3H), 3.07 (t, J=12.47 Hz, 1H), 2.19-2.07 (m, 1H), 2.01-1.49 (m, 4H), 1.07 (d, J=6.36 Hz, 3H), 0.92 (d, J=8.31 Hz, 2H), 0.66 (d, J=4.40 Hz, 2H).

### Example D-246: Synthesis of 3-[(1-methyl-1H-pyrazol-4-yl)amino]-5-[(2R,3R)-2-methyl-3-(2-methyl-1-benzofuran-5-amido)piperidin-1-yl]pyrazine-2-carboxamide (D-246)

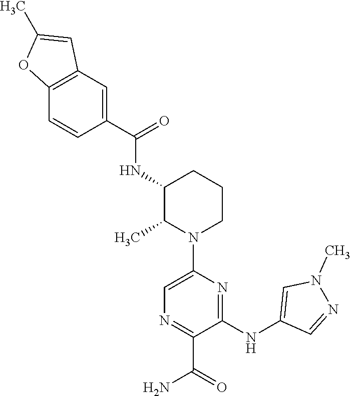 [SMILES: Cc1cc2cc(C(=O)N[C@@H]3CCCN(c4cnc(C(N)=O)c(Nc5cnn(C)c5)n4)[C@@H]3C)ccc2o1]

In a similar manner as described in Example 8, 3-[(1-methyl-1H-pyrazol-4-yl)amino]-5-[(2R,3R)-2-methyl-3-(2-methyl-1-benzofuran-5-amido)piperidin-1-yl]pyrazine-2-carboxamide (D-246) was prepared using 2-methyl-1-benzofuran-5-carboxylic acid. MS found for C25H28N8O3 as (M+H)⁺ 489.4.

¹H NMR (500 MHz, DMSO) δ 10.84 (s, 1H), 8.42 (d, J=6.65 Hz, 1H), 8.11 (d, J=1.57 Hz, 1H), 8.01 (s, 1H), 7.79 (dd, J=8.61, 1.96 Hz, 1H), 7.67 (br. s., 1H), 7.59-7.51 (m, 2H), 7.46 (s, 1H), 7.26 (br. s., 1H), 6.69 (s, 1H), 5.47-5.00 (m, 1H), 4.21-3.93 (m, 2H), 3.73 (s, 3H), 3.08 (t, J=13.11 Hz, 1H), 2.46 (d, J=0.78 Hz, 3H), 2.07-1.48 (m, 4H), 1.10 (d, J=7.04 Hz, 3H).

### Example D-247: Synthesis of 5-[(2R,3R)-3-(5-tert-butyl-1,2-oxazole-3-amido)-2-methylpiperidin-1-yl]-3-[(1-methyl-1H-pyrazol-4-yl)amino]pyrazine-2-carboxamide (D-247)

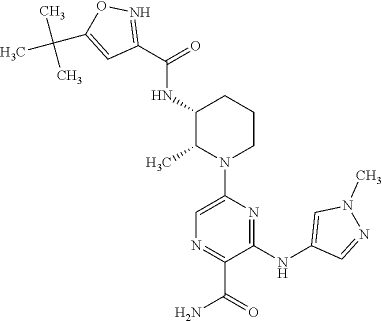 [SMILES: C[C@@H]1[C@H](NC(=O)c2cc(C(C)(C)C)on2)CCCN1c1cnc(C(N)=O)c(Nc2cnn(C)c2)n1]

In a similar manner as described in Example 8, 5-[(2R,3R)-3-(5-tert-butyl-1,2-oxazole-3-amido)-2-methylpiperidin-1-yl]-3-[(1-methyl-1H-pyrazol-4-yl)amino]pyrazine-2-carboxamide (D-247) was prepared using 5-tert-butylisoxazole-3-carboxylic acid. MS found for C23H31N9O3 as (M+H)⁺ 482.1.

¹H NMR (500 MHz, DMSO) δ 10.82 (s, 1H), 8.83 (d, J=6.36 Hz, 1H), 7.97 (br. s., 1H), 7.70 (br. s., 1H), 7.56 (s, 1H), 7.48 (s, 1H), 7.28 (br. s., 1H), 6.61 (s, 1H), 5.26 (br. s., 1H), 4.24-3.92 (m, 2H), 3.79 (s, 3H), 3.06 (t, J=12.96 Hz, 1H), 2.06-1.53 (m, 4H), 1.39-1.29 (m, 9H), 1.08 (d, J=6.36 Hz, 3H).

### Example D-248: Synthesis of 5-[(2R,3R)-3-{4-[(2-methoxyethyl)(methyl)amino]benzamido}-2-methylpiperidin-1-yl]-3-[(1-methyl-1H-pyrazol-4-yl)amino]pyrazine-2-carboxamide (D-248)

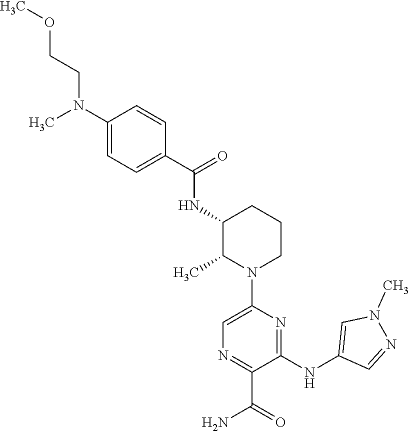 [SMILES: COCCN(C)c1ccc(C(=O)N[C@@H]2CCCN(c3cnc(C(N)=O)c(Nc4cnn(C)c4)n3)[C@@H]2C)cc1]

In a similar manner as described in Example 8, 5-[(2R,3R)-3-{4-[(2-methoxyethyl)(methyl)amino]benzamido}-2-methylpiperidin-1-yl]-3-[(1-methyl-1H-pyrazol-4-yl)amino]pyrazine-2-carboxamide (D-248) was prepared using 4-[(2-methoxyethyl)(methyl)amino]benzoic acid. MS found for C26H35N9O3 as (M+H)⁺ 522.4.

¹H NMR (500 MHz, DMSO) δ 10.86 (s, 1H), 8.10-7.98 (m, 2H), 7.79 (d, J=9.00 Hz, 2H), 7.68 (br.s., 1H), 7.56 (s, 1H), 7.46 (s, 1H), 7.26 (br. s., 1H), 6.72 (d, J=9.00 Hz, 2H), 5.55-5.01 (m, 1H), 4.20-3.93 (m, 2H), 3.78 (s, 3H), 3.63-3.53 (m, 2H), 3.53-3.46 (m, 2H), 3.25 (s, 3H), 3.13-3.02 (m, 1H), 2.98 (s, 3H), 2.05-1.51 (m, 4H), 1.07 (d, J=7.04 Hz, 3H).

### Example D-249: Synthesis of 3-[(1-methyl-1H-pyrazol-4-yl)amino]-5-[(2R,3R)-2-methyl-3-[4-(2,2,2-trifluoro-1-hydroxyethyl)benzamido]piperidin-1-yl]pyrazine-2-carboxamide. Cis, Diastereoisomer 1 (D-249)

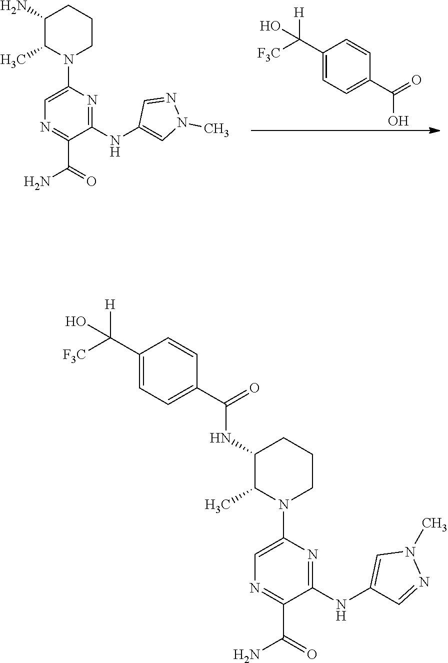 [SMILES: C[C@@H]1[C@H](N)CCCN1c1cnc(C(N)=O)c(Nc2cnn(C)c2)n1 ; O=C(O)c1ccc(C(O)C(F)(F)F)cc1 ; C[C@@H]1[C@H](NC(=O)c2ccc(C(O)C(F)(F)F)cc2)CCCN1c1cnc(C(N)=O)c(Nc2cnn(C)c2)n1]

To a solution of 5-[(2R,3R)-3-amino-2-methylpiperidin-1-yl]-3-[(1-methyl-1H-pyrazol-4-yl)amino]pyrazine-2-carboxamide (192 mg, 0.58 mmol) in DMF (4 mL) DIPEA (0.3 mL), 4-(2,2,2-trifluoro-1-hydroxyethyl)benzoic acid (144 mg, 0.64 mmol) and PyBOP (420 mg, 0.81 mmol) were added. The mixture was stirred at room temperature for for 2 hr then it was partitioned between ethyl acetate/diethyl ether and water. The organic phase was dried over Na₂SO₄, filtered and concentrated. The obtained crude was purified first by silica flash chromatography with 50 to 100% ethyl acetate in cyclohexane, then by chiral chromatography to give 3-[(1-methyl-1H-pyrazol-4-yl)amino]-5-[(2R,3R)-2-methyl-3-[4-(2,2,2-trifluoro-1-hydroxyethyl)benzamido]piperidin-1-yl]pyrazine-2-carboxamide. Cis, diastereoisomer 1 (D-249) 90 mg, 29% yield as a yellow solid. MS found for C24H27F3N8O3 as (M+H)+533.3.

¹H NMR (500 MHz, DMSO) δ 10.87 (s, 1H), 8.50 (d, J=6.59 Hz, 1H), 8.02 (br. s., 1H), 7.93 (d, J=8.23 Hz, 2H), 7.70 (br. s., 1H), 7.61 (d, J=8.23 Hz, 2H), 7.58-7.55 (m, 1H), 7.47 (s, 1H), 7.28 (br. s., 1H), 6.97 (br. s., 1H), 5.27 (m, J=6.30 Hz, 2H), 4.26-3.92 (m, 2H), 3.75 (s, 3H), 3.14-3.03 (m, 1H), 1.99-1.54 (m, 4H), 1.10 (d, J=6.86 Hz, 3H).

### Example D-250: Synthesis of 3-[(1-methyl-1H-pyrazol-4-yl)amino]-5-[(2R,3R)-2-methyl-3-[4-(2,2,2-trifluoro-1-hydroxyethyl)benzamido]piperidin-1-yl]pyrazine-2-carboxamide. Cis, Diastereoisomer 2 (D-250)

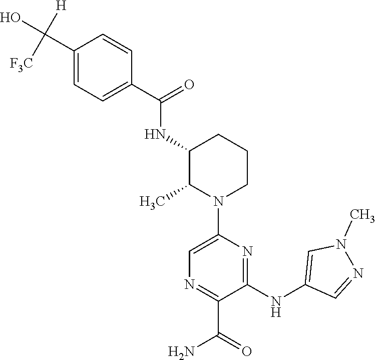 [SMILES: C[C@@H]1[C@H](NC(=O)c2ccc(C(O)C(F)(F)F)cc2)CCCN1c1cnc(C(N)=O)c(Nc2cnn(C)c2)n1]

In a similar manner as described in Example D-249 (PCI-58520), 3-[(1-methyl-1H-pyrazol-4-yl)amino]-5-[(2R,3R)-2-methyl-3-[4-(2,2,2-trifluoro-1-hydroxyethyl)benzamido]piperidin-1-yl]pyrazine-2-carboxamide. Cis, diastereoisomer 2 (D-250) was prepared using 4-(2,2,2-trifluoro-1-hydroxyethyl)benzoic acid. MS found for C24H27F3N8O3 as (M+H)⁺ 533.3.

¹H NMR (500 MHz, DMSO) δ 10.87 (s, 1H), 8.50 (d, J=6.59 Hz, 1H), 8.02 (br. s., 1H), 7.93 (d, J=8.23 Hz, 2H), 7.70 (br. s., 1H), 7.61 (d, J=8.23 Hz, 2H), 7.58-7.55 (m, 1H), 7.47 (s, 1H), 7.28 (br. s., 1H), 6.97 (br. s., 1H), 5.27 (m, J=6.30 Hz, 2H), 4.26-3.92 (m, 2H), 3.75 (s, 3H), 3.14-3.03 (m, 1H), 1.99-1.54 (m, 4H), 1.10 (d, J=6.86 Hz, 3H).

### Example D-251: Synthesis of 5-[(2R,3R)-3-[4-(2-fluoropropan-2-yl)benzamido]-2-methylpiperidin-1-yl]-3-[(1-methyl-1H-pyrazol-4-yl)amino]pyrazine-2-carboxamide (D-251)

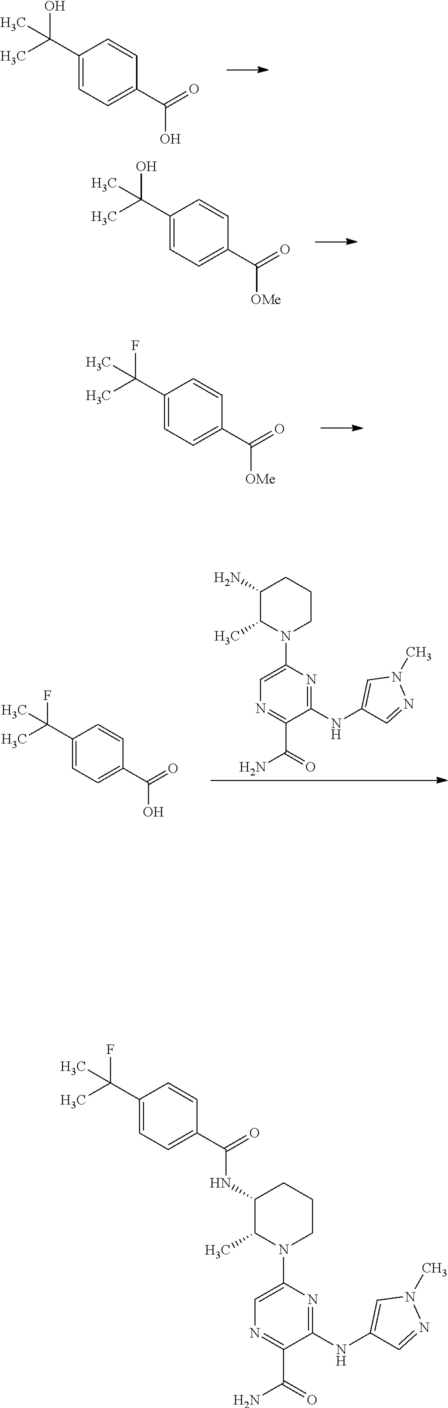

To a solution of 4-(2-hydroxypropan-2-yl)benzoic acid (200 mg, 1.11 mmol) in DCM/MeOH (5/3 mL) 2M TMSCHN₂ in cyclohexane (0.85 mL, 1.67 mmol) was added at 0° C. The mixture was left to reach room temperature in about 1 h then it was evaporated to dryness to give methyl 4-(2-hydroxypropan-2-yl)benzoate (212 mg, 98% yield). MS found for C11H14O3 as (M+H)⁺ 195.0.

To a solution of methyl 4-(2-hydroxypropan-2-yl)benzoate (100 mg, 0.515 mmol) in DCM (3 mL) DAST (90 μL, 0.67 mmol) was added at about −10/0° C. The mixture was stirred at this temperature for 1 h then it was quenched with water. It was partitioned between DCM and water. The organic phase was dried over Na₂SO₄, filtered and concentrated. The obtained crude was purified by silica flash chromatography with 0 to 50% ethyl acetate in cyclohexane to give methyl 4-(2-fluoropropan-2-yl)benzoate (56 mg, 55% yield) as a colorless oil. MS found for C11H13FO2 as (M+H)⁺ 197.1.

To a solution of 4-(2-fluoropropan-2-yl)benzoate (56 mg, 0.285 mmol) in THF/MeOH/H₂O (1/1/1 mL) LiOH.H₂O (24 mg, 0.57 mmol) was added. The mixture was stirred at room temperature overnight then it was treated with 1N HCl to pH about 2. It was extracted with ethyl acetate. The combined organic phases were dried over Na₂SO₄, filtered and concentrated. The obtained crude was purified by C18 flash chromatography with 0 to 100% acetonitrile in water 0.1% HCOOH to give 4-(2-fluoropropan-2-yl)benzoic acid (27 mg, 52% yield) as a white solid. MS found for C10H11FO2 as (M+H)⁺ 183.1.

In a similar manner as described in Example 8, 5-[(2R,3R)-3-[4-(2-fluoropropan-2-yl)benzamido]-2-methylpiperidin-1-yl]-3-[(1-methyl-1H-pyrazol-4-yl)amino]pyrazine-2-carboxamide (D-251) was prepared using 4-(2-fluoropropan-2-yl)benzoic acid. MS found for C25H31FN8O2 as (M+H)⁺ 495.4.

¹H NMR (500 MHz, DMSO) δ 10.88 (s, 1H), 8.45 (d, J=6.59 Hz, 1H), 8.03 (br.s., 1H), 7.93 (d, J=8.23 Hz, 2H), 7.69 (br. s., 1H), 7.57 (s, 1H), 7.53 (d, J=8.51 Hz, 2H), 7.50-7.46 (m, 1H), 7.28 (br. s., 1H), 5.65-5.06 (m, 1H), 4.26-3.94 (m, 2H), 3.77 (s, 3H), 3.17-3.00 (m, 1H), 1.72-1.63 (m, 6H), 2.04-1.55 (m, 4H), 1.10 (d, J=6.86 Hz, 3H).

### Example D-252: Synthesis of 5-[(2R,3R)-3-(3-cyclopropyl-1H-pyrazole-5-amido)-2-methylpiperidin-1-yl]-3-[(1-methyl-1H-pyrazol-4-yl)amino]pyrazine-2-carboxamide (D-252)

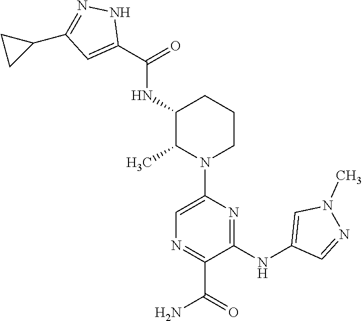 [SMILES: C[C@@H]1[C@H](NC(=O)c2cc(C3CC3)n[nH]2)CCCN1c1cnc(C(N)=O)c(Nc2cnn(C)c2)n1]

In a similar manner as described in Example 8, 5-[(2R,3R)-3-(3-cyclopropyl-1H-pyrazole-5-amido)-2-methylpiperidin-1-yl]-3-[(1-methyl-1H-pyrazol-4-yl)amino]pyrazine-2-carboxamide (D-252) was prepared using 3-cyclopropyl-1H-pyrazole-5-carboxylic acid. MS found for C22H28N10O2 as (M+H)⁺ 465.4.

¹H NMR (500 MHz, DMSO) δ 13.12-12.93 (m, 1H), 10.85 (s, 1H), 8.10-7.89 (m, 1H), 8.36-7.88 (m, 1H), 7.69 (br. s., 1H), 7.60-7.52 (m, 1H), 7.45 (s, 1H), 7.27 (br. s., 1H), 6.72-6.29 (m, 1H), 5.28 (br. s., 1H), 4.24-3.89 (m, 2H), 3.86-3.68 (m, 3H), 3.13-2.98 (m, 1H), 2.06-1.76 (m, 3H), 1.74-1.50 (m, 2H), 1.13-1.01 (m, 3H), 1.00-0.83 (m, 2H), 0.77-0.57 (m, 2H).

### Example D-253: Synthesis of 5-[(2R,3R)-3-(5-cyclopropyl-1-methyl-1H-pyrazole-3-amido)-2-methylpiperidin-1-yl]-3-[(1-methyl-1H-pyrazol-4-yl)amino]pyrazine-2-carboxamide (D-253)

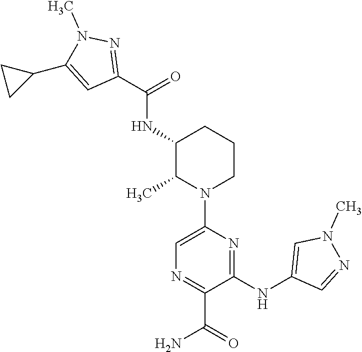 [SMILES: C[C@@H]1[C@H](NC(=O)c2cc(C3CC3)n(C)n2)CCCN1c1cnc(C(N)=O)c(Nc2cnn(C)c2)n1]

In a similar manner as described in Example 8, 5-[(2R,3R)-3-(5-cyclopropyl-1-methyl-1H-pyrazole-3-amido)-2-methylpiperidin-1-yl]-3-[(1-methyl-1H-pyrazol-4-yl)amino]pyrazine-2-carboxamide (D-253) was prepared using 5-cyclopropyl-1-methyl-1H-pyrazole-3-carboxylic acid. MS found for C23H30N10O2 as (M+H)⁺ 479.4.

¹H NMR (400 MHz, DMSO) δ 10.85 (s, 1H), 8.01 (s, 1H), 7.93 (d, J=7.04 Hz, 1H), 7.68 (br. s., 1H), 7.54 (s, 1H), 7.45 (s, 1H), 7.27 (br. s., 1H), 6.31 (s, 1H), 5.46-5.03 (m, 1H), 4.17-3.93 (m, 2H), 3.90 (s, 3H), 3.79 (s, 3H), 3.11-2.98 (m, 1H), 2.07-1.47 (m, 5H), 1.05 (d, J=7.04 Hz, 3H), 1.00-0.92 (m, 2H), 0.59-0.71 (m, 2H).

### Example D-254: Synthesis of N-[(2R,3R)-1-{5-carbamoyl-6-[(1-methyl-1H-pyrazol-4-yl)amino]pyrazin-2-yl}-2-methylpiperidin-3-yl]-1-methyl-1H-indazole-5-carboxamide (D-254)

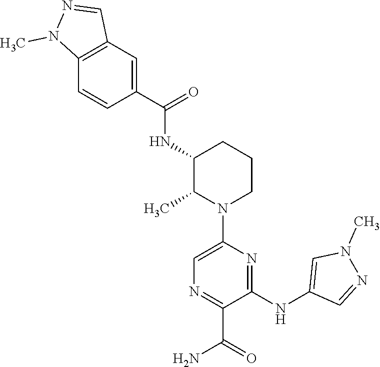 [SMILES: C[C@@H]1[C@H](NC(=O)c2ccc3c(cnn3C)c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1-methyl-1H-indazole-5-carboxamide (D-254) was prepared using 1-methyl-1H-indazole-5-carboxylic acid. MS found for C24H28N10O2 as (M+H)⁺ 489.1.

¹H NMR (400 MHz, DMSO) δ 10.86 (s, 1H), 8.48 (d, J=7.04 Hz, 1H), 8.42 (s, 1H), 8.22 (s, 1H), 8.04 (s, 1H), 7.97 (dd, J=9.00, 1.56 Hz, 1H), 7.79-7.65 (m, 2H), 7.58 (s, 1H), 7.47 (s, 1H), 7.28 (br.s., 1H), 5.52-5.09 (m, 1H), 4.09 (s, 5H), 3.74 (s, 3H), 3.18-3.03 (m, 1H), 1.99 (s, 4H), 1.13 (d, J=6.65 Hz, 3H).

### Example D-255: Synthesis of N-[(2R,3R)-1-{5-carbamoyl-6-[(1-methyl-1H-pyrazol-4-yl)amino]pyrazin-2-yl}-2-methylpiperidin-3-yl]-1-(propan-2-yl)-1H-1,2,3-benzotriazole-5-carboxamide (D-255)

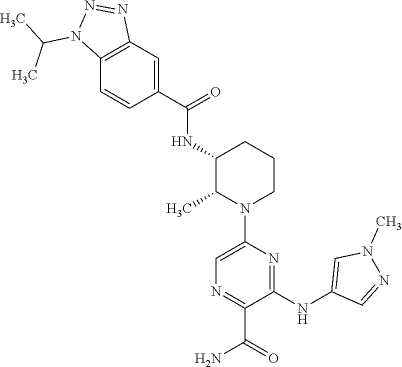 [SMILES: CC(C)n1nnc2cc(C(=O)N[C@@H]3CCCN(c4cnc(C(N)=O)c(Nc5cnn(C)c5)n4)[C@@H]3C)ccc21]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1-(propan-2-yl)-1H-1,2,3-benzotriazole-5-carboxamide (D-255) was prepared using 1-(propan-2-yl)-1H-1,2,3-benzotriazole-5-carboxylic acid. MS found for C25H31N11O2 as (M+H)⁺ 518.2.

¹H NMR (500 MHz, DMSO) δ 10.88 (s, 1H), 8.69 (s, 1H), 8.63 (d, J=6.86 Hz, 1H), 8.15-8.07 (m, 1H), 8.06-7.99 (m, 2H), 7.76-7.67 (m, 1H), 7.59 (s, 1H), 7.48 (s, 1H), 7.29 (br. s., 1H), 5.71-4.96 (m, 2H), 4.25-4.05 (m, 2H), 3.76 (s, 3H), 3.18-3.06 (m, 1H), 2.09-1.74 (m, 3H), 1.71-1.56 (m, 7H), 1.14 (d, J=6.86 Hz, 3H).

### Example D-256: Synthesis of 5-[(2R,3R)-3-[4-(1,2-dihydroxypropan-2-yl)benzamido]-2-methylpiperidin-1-yl]-3-[(1-methyl-1H-pyrazol-4-yl)amino]pyrazine-2-carboxamide. Cis, diastereoisomer 1 (D-256)

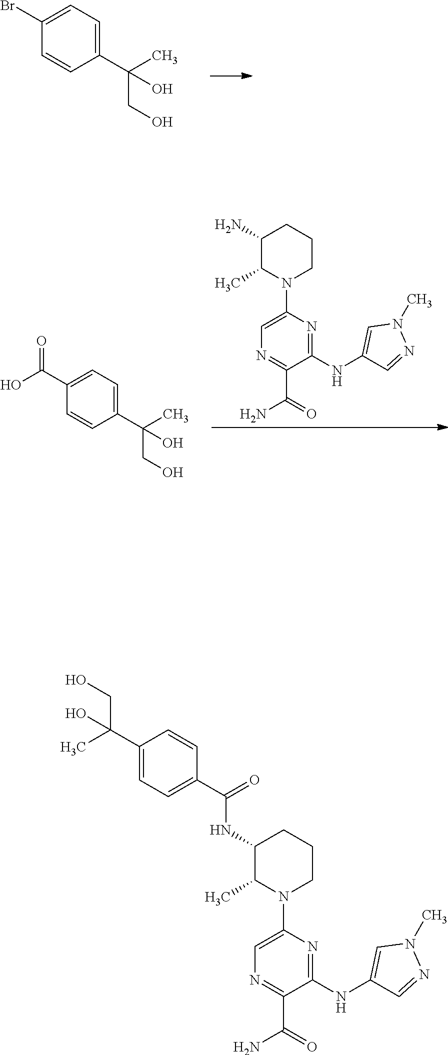 [SMILES: CC(O)(CO)c1ccc(Br)cc1 ; CC(O)(CO)c1ccc(C(=O)O)cc1 ; C[C@@H]1[C@H](N)CCCN1c1cnc(C(N)=O)c(Nc2cnn(C)c2)n1 ; C[C@@H]1[C@H](NC(=O)c2ccc(C(C)(O)CO)cc2)CCCN1c1cnc(C(N)=O)c(Nc2cnn(C)c2)n1]

In a similar manner as described in Example D-235, PCI-58487, 4-(1,2-dihydroxypropan-2-yl)benzoic acid was prepared using 2-(4-bromophenyl)propane-1,2-diol. MS found for C10H12O4 as (M+H)⁺ 179.0.

In a similar manner as described in Example 8, 5-[(2R,3R)-3-[4-(1,2-dihydroxypropan-2-yl)benzamido]-2-methylpiperidin-1-yl]-3-[(1-methyl-1H-pyrazol-4-yl)amino]pyrazine-2-carboxamide cis, diastereoisomer 1 (D-256) was prepared using 4-(1,2-dihydroxypropan-2-yl)benzoic acid. MS found for C25H32N8O4 as (M+H)⁺ 509.4.

¹H NMR (500 MHz, DMSO) δ 10.88 (s, 1H), 8.36 (d, J=6.85 Hz, 1H), 8.04 (br. s., 1H), 7.85 (d, J=8.31 Hz, 2H), 7.69 (br. s., 1H), 7.62-7.51 (m, 3H), 7.47 (s, 1H), 7.28 (br. s., 1H), 5.59-5.11 (m, 1H), 5.00 (s, 1H), 4.72 (t, J=5.62 Hz, 1H), 4.27-3.95 (m, 2H), 3.78 (s, 3H), 3.43 (dd, J=5.62, 1.71 Hz, 2H), 3.08 (t, J=12.23 Hz, 1H), 2.11-1.53 (m, 4H), 1.41 (s, 3H), 1.09 (d, J=6.85 Hz, 3H).

### Example D-257: Synthesis of 5-[(2R,3R)-3-(2-cyclopropyl-1,3-oxazole-4-amido)-2-methylpiperidin-1-yl]-3-[(1-methyl-1H-pyrazol-4-yl)amino]pyrazine-2-carboxamide (D-257)

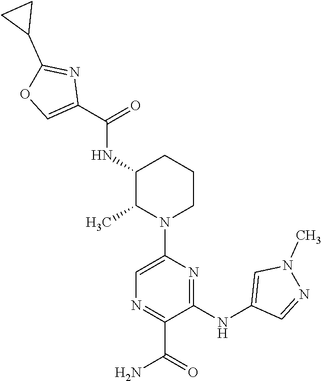 [SMILES: C[C@@H]1[C@H](NC(=O)c2coc(C3CC3)n2)CCCN1c1cnc(C(N)=O)c(Nc2cnn(C)c2)n1]

In a similar manner as described in Example 8, 5-[(2R,3R)-3-(2-cyclopropyl-1,3-oxazole-4-amido)-2-methylpiperidin-1-yl]-3-[(1-methyl-1H-pyrazol-4-yl)amino]pyrazine-2-carboxamide (D-257) was prepared using 2-cyclopropyl-1,3-oxazole-4-carboxylic acid. MS found for C22H27N9O3 as (M+H)⁺ 466.4.

¹H NMR (500 MHz, DMSO) δ 10.85 (s, 1H), 8.47 (s, 1H), 8.12 (d, J=7.34 Hz, 1H), 8.01 (s, 1H), 7.68 (br. s., 1H), 7.55 (s, 1H), 7.45 (s, 1H), 7.27 (br. s., 1H), 5.44-5.10 (m, 1H), 4.20-3.91 (m, 2H), 3.79 (s, 3H), 3.15-2.97 (m, 1H), 2.24-2.12 (m, 1H), 2.07-1.51 (m, 4H), 1.07 (s, 7H).

### Example D-258: Synthesis of 5-[(2R,3R)-3-(2,2-dimethyl-3,4-dihydro-2H-1-benzopyran-6-amido)-2-methylpiperidin-1-yl]-3-[(1-methyl-1H-pyrazzol-4-yl)amino]pyrazine-2-carboxamide (D-258)

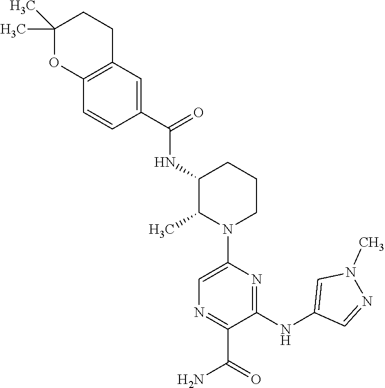 [SMILES: C[C@@H]1[C@H](NC(=O)c2ccc3c(c2)CCC(C)(C)O3)CCCN1c1cnc(C(N)=O)c(Nc2cnn(C)c2)n1]

In a similar manner as described in Example 8, 5-[(2R,3R)-3-(2,2-dimethyl-3,4-dihydro-2H-1-benzopyran-6-amido)-2-methylpiperidin-1-yl]-3-[(1-methyl-1H-pyrazol-4-yl)amino]pyrazine-2-carboxamide (D-258) was prepared using 2,2-dimethyl-3,4-dihydro-2H-1-benzopyran-6-carboxylic acid. MS found for C₂₇H₃₄N₈O₃ as (M+H)⁺ 519.2.

¹H NMR (500 MHz, DMSO) δ 10.93-10.77 (m, 1H), 8.20 (d, J=6.85 Hz, 1H), 8.02 (br. s., 1H), 7.73 (s, 1H), 7.71-7.64 (m, 2H), 7.56 (s, 1H), 7.47 (s, 1H), 7.28 (br. s., 1H), 6.77 (d, J=8.31 Hz, 1H), 5.30 (br. s., 1H), 4.24-3.93 (m, 2H), 3.77 (s, 3H), 3.08 (t, J=12.23 Hz, 1H), 2.79 (t, J=6.85 Hz, 2H), 1.80 (t, J=6.60 Hz, 2H), 1.98-1.53 (m, 4H), 1.30 (s, 6H), 1.08 (d, J=6.85 Hz, 3H).

### Example D-259: Synthesis of N-[(2R,3R)-1-{5-carbamoyl-6-[(1-methyl-1H-pyrazol-4-yl)amino]pyrazin-2-yl}-2-methylpiperidin-3-yl]-1-methyl-1H-indazole-6-carboxamide (D-259)

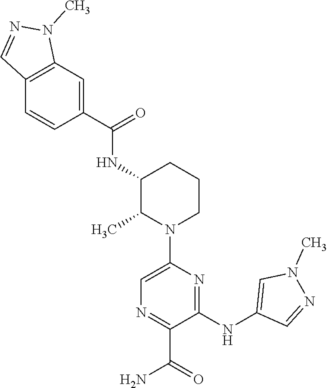 [SMILES: C[C@@H]1[C@H](NC(=O)c2ccc3cnn(C)c3c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1-methyl-1H-indazole-6-carboxamide (D-259) was prepared using 1-methyl-1H-indazole-6-carboxylic acid. MS found for C24H28N10O2 as (M+H)⁺ 489.3.

¹H NMR (500 MHz, DMSO) δ 10.88 (s, 1H), 8.55 (d, J=6.72 Hz, 1H), 8.23 (s, 1H), 8.13 (s, 1H), 8.05 (br. s., 1H), 7.85 (d, J=8.37 Hz, 1H), 7.75-7.64 (m, 2H), 7.59 (s, 1H), 7.48 (s, 1H), 7.29 (br. s., 1H), 5.36 (br. s., 1H), 4.22-4.05 (m, 5H), 3.76 (s, 3H), 3.12 (t, J=12.21 Hz, 1H), 2.08-1.95 (m, 1H), 1.89 (d, J=13.17 Hz, 1H), 1.77 (d, J=10.02 Hz, 1H), 1.71-1.57 (m, 1H), 1.14 (d, J=6.86 Hz, 3H).

### Example D-260: Synthesis of 5-[(2R,3R)-3-{4-[1-(hydroxymethyl)cyclopropyl]benzamido}-2-methylpiperidin-1-yl]-3-[(1-methyl-1H-pyrazol-4-yl)amino]pyrazine-2-carboxamide (D-260)

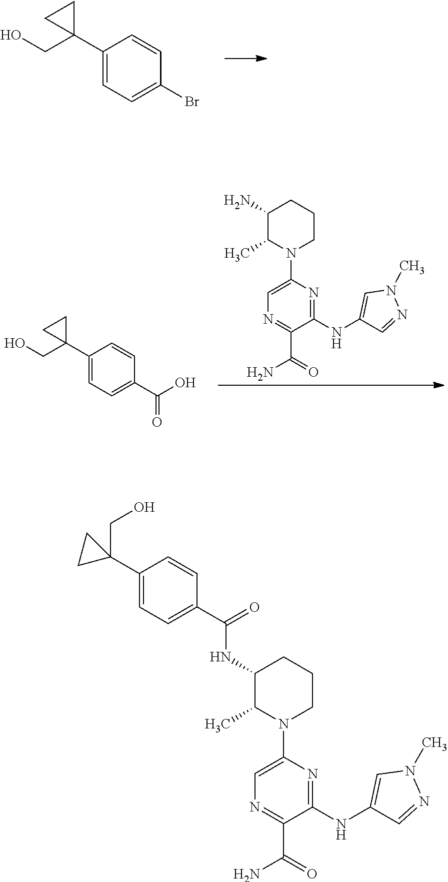 [SMILES: OCC1(c2ccc(Br)cc2)CC1 ; O=C(O)c1ccc(C2(CO)CC2)cc1 ; C[C@@H]1[C@H](N)CCCN1c1cnc(C(N)=O)c(Nc2cnn(C)c2)n1 ; C[C@@H]1[C@H](NC(=O)c2ccc(C3(CO)CC3)cc2)CCCN1c1cnc(C(N)=O)c(Nc2cnn(C)c2)n1]

In a similar manner as described in Example D-235, 4-[1-(hydroxymethyl)cyclopropyl]benzoic acid was prepared using (1-(4-bromophenyl)cyclopropyl)methanol. MS found for C11H12O3 as (M+H)⁺ 193.1.

In a similar manner as described in Example 8, 5-[(2R,3R)-3-{4-[1-(hydroxymethyl)cyclopropyl]benzamido}-2-methylpiperidin-1-yl]-3-[(1-methyl-1H-pyrazol-4-yl)amino]pyrazine-2-carboxamide (D-260) was prepared using 4-[1-(hydroxymethyl)cyclopropyl]benzoic acid. MS found for C26H32N8O3 as (M+H)⁺ 505.2.

¹H NMR (500 MHz, DMSO) δ 10.87 (s, 1H), 8.35 (d, J=6.86 Hz, 1H), 8.03 (br. s., 1H), 7.83 (d, J=8.23 Hz, 2H), 7.69 (br. s., 1H), 7.56 (s, 1H), 7.47 (s, 1H), 7.39 (d, J=8.37 Hz, 2H), 7.28 (br. s., 1H), 5.33 (br. s., 1H), 4.72 (t, J=5.63 Hz, 1H), 4.23-3.96 (m, 2H), 3.77 (s, 3H), 3.57 (d, J=5.63 Hz, 2H), 3.08 (t, J=12.21 Hz, 1H), 2.03-1.90 (m, 1H), 1.86 (d, J=13.04 Hz, 1H), 1.71 (d, J=10.15 Hz, 1H), 1.66-1.54 (m, 1H), 1.08 (d, J=6.72 Hz, 3H), 0.94-0.85 (m, 2H), 0.84-0.76 (m, 2H).

### Example D-261: Synthesis of N-[(2R,3R)-1-{5-carbamoyl-6-[(1-methyl-1H-pyrazol-4-yl)amino]pyrazin-2-yl}-2-methylpiperidin-3-yl]-2-methyl-1H-1,3-benzodiazole-5-carboxamide (D-261)

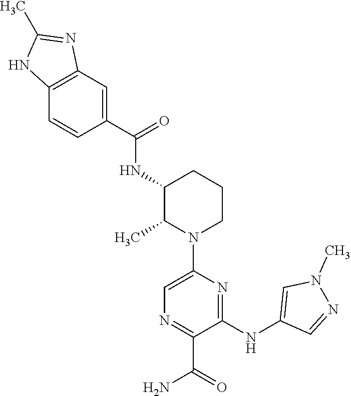 [SMILES: Cc1nc2cc(C(=O)N[C@@H]3CCCN(c4cnc(C(N)=O)c(Nc5cnn(C)c5)n4)[C@@H]3C)ccc2[nH]1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-2-methyl-1H-1,3-benzodiazole-5-carboxamide (D-261) was prepared using 2-methyl-1H-1,3-benzodiazole-5-carboxylic acid. MS found for C24H28N10O2 as (M+H)⁺ 489.3.

¹H NMR (500 MHz, DMSO) δ 12.44 (br. s., 1H), 10.87 (s, 1H), 8.39 (d, J=6.36 Hz, 1H), 8.21-7.91 (m, 2H), 7.75 (d, J=8.31 Hz, 1H), 7.69 (br. s., 1H), 7.58 (s, 1H), 7.47 (s, 2H), 7.28 (br. s., 1H), 5.37 (br. s., 1H), 3.94-4.26 (m, 2H), 3.76 (s, 3H), 3.09 (t, J=12.23 Hz, 1H), 2.53-2.50 (m, 3H), 2.10-1.52 (m, 4H), 1.07-1.15 (m, 3H).

### Example D-262: Synthesis of N-[(2R,3R)-1-{5-carbamoyl-6-[(1-methyl-1H-pyrazol-4-yl)amino]pyrazin-2-yl}-2-methylpiperidin-3-yl]-1H-1,3-benzodiazole-5-carboxamide (D-262)

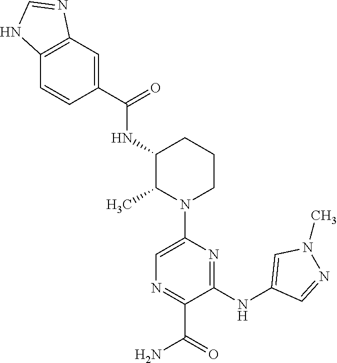 [SMILES: C[C@@H]1[C@H](NC(=O)c2ccc3[nH]cnc3c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-1H-1,3-benzodiazole-5-carboxamide (D-262) was prepared using 5-benzimidazolecarboxylic acid. MS found for C23H26N10O2 as (M+H)⁺ 475.3.

¹H NMR (500 MHz, DMSO) δ 13.36-11.90 (m, 1H), 10.87 (s, 1H), 8.44 (d, J=4.89 Hz, 1H), 8.35 (s, 1H), 8.29-7.96 (m, 2H), 7.88-7.60 (m, 3H), 7.58 (s, 1H), 7.47 (s, 1H), 7.28 (br. s., 1H), 5.35 (br. s., 1H), 4.28-3.94 (m, 2H), 3.76 (s, 3H), 3.10 (t, J=12.23 Hz, 1H), 2.07-1.54 (m, 4H), 1.05-1.19 (m, 3H).

### Example D-263: Synthesis of 3-[(1-methyl-1H-pyrazol-4-yl)amino]-5-[(2R,3R)-2-methyl-3-{4H,5H,6H-pyrrolo[1,2-b]pyrazole-2-amido}piperidin-1-yl]pyrazine-2-carboxamide (D-263)

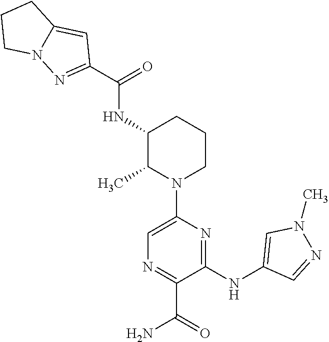 [SMILES: C[C@@H]1[C@H](NC(=O)c2cc3n(n2)CCC3)CCCN1c1cnc(C(N)=O)c(Nc2cnn(C)c2)n1]

In a similar manner as described in Example 8, 3-[(1-methyl-1H-pyrazol-4-yl)amino]-5-[(2R,3R)-2-methyl-3-{4H, 5H,6H-pyrrolo[1,2-b]pyrazole-2-amido}piperidin-1-yl]pyrazine-2-carboxamide (D-263) was prepared using 5,6-dihydro-4H-pyrrolo[1,2-b]pyrazole-2-carboxylic acid. MS found for C22H28N10O2 as (M+H)⁺ 465.1.

¹H NMR (500 MHz, DMSO) δ 10.86 (s, 1H), 8.10-7.96 (m, 2H), 7.68 (br. s., 1H), 7.55 (s, 1H), 7.45 (s, 1H), 7.26 (br. s., 1H), 6.42 (s, 1H), 5.31 (br. s., 1H), 4.27-3.93 (m, 4H), 3.81 (s, 3H), 3.13-2.99 (m, 1H), 2.87 (t, J=7.34 Hz, 2H), 2.62-2.53 (m, 2H), 2.09-1.48 (m, 4H), 1.05 (d, J=7.02 Hz, 3H).

### Example D-264: Synthesis of 5-[(2R,3R)-3-(2-cyclopropyl-1H-imidazole-4-amido)-2-methylpiperidin-1-yl]-3-[(1-methyl-1H-pyrazol-4-yl)amino]pyrazine-2-carboxamide (D-264)

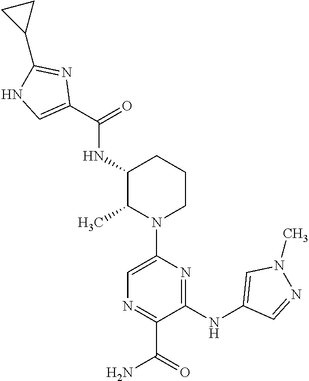 [SMILES: C[C@@H]1[C@H](NC(=O)c2c[nH]c(C3CC3)n2)CCCN1c1cnc(C(N)=O)c(Nc2cnn(C)c2)n1]

In a similar manner as described in Example 8, 5-[(2R,3R)-3-(2-cyclopropyl-1H-imidazole-4-amido)-2-methylpiperidin-1-yl]-3-[(1-methyl-1H-pyrazol-4-yl)amino]pyrazine-2-carboxamide (D-264) was prepared using 2-cyclopropyl-1H-imidazole-4-carboxylic acid hydrochloride. MS found for C22H28N10O2 as (M+H)⁺ 465.3.

¹H NMR (500 MHz, DMSO) δ 12.46-11.99 (m, 1H), 10.83 (s, 1H), 8.03 (br. s., 1H), 7.67 (br. s., 1H), 7.53 (s, 1H), 7.48 (s, 2H), 7.43 (s, 1H), 7.25 (br. s., 1H), 5.27 (br. s., 1H), 3.94 (d, J=4.89 Hz, 2H), 3.85-3.67 (m, 3H), 3.04 (t, J=12.23 Hz, 1H), 2.05-1.49 (m, 5H), 1.04 (d, J=6.85 Hz, 3H), 0.95-0.79 (n, 4H).

### Example D-265: Synthesis of 5-[(2R,3R)-3-[4-(1,2-dihydroxypropan-2-yl)benzamido]-2-methylpiperidin-1-yl]-3-[(1-methyl-1H-pyrazol-4-yl)amino]pyrazine-2-carboxamide. Cis, diastereoisomer 2 (D-265)

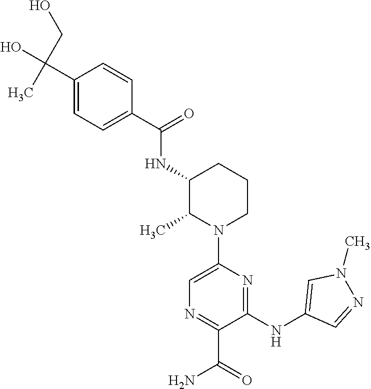 [SMILES: C[C@@H]1[C@H](NC(=O)c2ccc(C(C)(O)CO)cc2)CCCN1c1cnc(C(N)=O)c(Nc2cnn(C)c2)n1]

In a similar manner as described in Example D-256, 5-[(2R,3R)-3-[4-(1,2-dihydroxypropan-2-yl)benzamido]-2-methylpiperidin-1-yl]-3-[(1-methyl-1H-pyrazol-4-yl)amino]pyrazine-2-carboxamide. Cis, diastereoisomer 2 (D-265) was prepared using 4-(1,2-dihydroxypropan-2-yl)benzoic acid. MS found for C25H32N8O4 as (M+H)⁺ 509.4.

¹H NMR (500 MHz, DMSO) δ 10.87 (s, 1H), 8.37 (d, J=6.86 Hz, 1H), 8.04 (s, 1H), 7.85 (d, J=8.23 Hz, 2H), 7.69 (s, 1H), 7.61-7.53 (m, 3H), 7.47 (s, 1H), 7.28 (s, 1H), 5.52-5.13 (m, 1H), 5.01 (br. s., 1H), 4.83-4.65 (m, 1H), 4.18-3.95 (m, 2H), 3.78 (s, 3H), 3.45-3.41 (m, 2H), 3.14-3.01 (m, 1H), 2.05-1.53 (m, 4H), 1.41 (s, 3H), 1.12-1.05 (m, 3H).

### Example D-266: Synthesis of 5-[(2R,3R)-3-{imidazo[1,2-a]pyridine-6-amido}-2-methylpiperidin-1-yl]-3-[(1-methyl-1H-pyrazol-4-yl)amino]pyrazine-2-carboxamide (D-266)

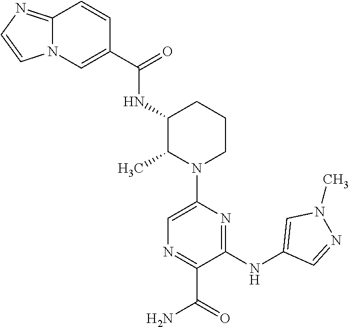 [SMILES: C[C@@H]1[C@H](NC(=O)c2ccc3nccn3c2)CCCN1c1cnc(C(N)=O)c(Nc2cnn(C)c2)n1]

In a similar manner as described in Example 8, 5-[(2R,3R)-3-{imidazo[1,2-a]pyridine-6-amido}-2-methylpiperidin-1-yl]-3-[(1-methyl-1H-pyrazol-4-yl)amino]pyrazine-2-carboxamide (D-266) was prepared using imidazole[1,2-A]pyridine-6-carboxylic acid. MS found for C23H26N10O2 as (M+H)⁺ 475.2.

¹H NMR (500 MHz, DMSO) δ 10.87 (s, 1H), 9.19 (d, J=1.57 Hz, 1H), 8.58 (d, J=7.04 Hz, 1H), 8.12 (s, 1H), 8.01 (s, 1H), 7.81-7.63 (m, 4H), 7.58 (s, 1H), 7.49 (s, 1H), 7.28 (d, J=1.96 Hz, 1H), 5.50-5.07 (m, 1H), 4.27-3.96 (m, 2H), 3.76 (s, 3H), 3.16-3.05 (m, 1H), 2.04-1.54 (m, 4H), 1.14 (d, J=7.04 Hz, 3H).

### Example D-267: Synthesis of N-[(2R,3R)-1-{5-carbamoyl-6-[(1-methyl-1H-pyrazol-4-yl)amino]pyrazin-2-yl}-2-methylpiperidin-3-yl]-2-methyl-2H-indazole-5-carboxamide (D-267)

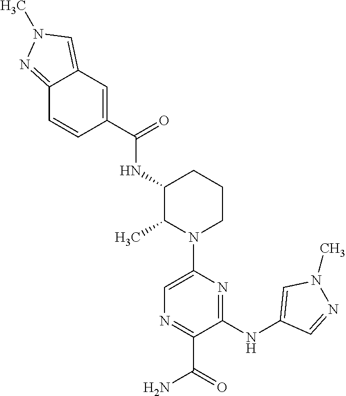 [SMILES: C[C@@H]1[C@H](NC(=O)c2ccc3nn(C)cc3c2)CCCN1c1cnc(C(N)=O)c(Nc2cnn(C)c2)n1]

In a similar manner as described in Example 8, N-[(2R,3R)-1-{5-carbamoyl-6-[(1-methyl-1H-pyrazol-4-yl)amino]pyrazin-2-yl}-2-methylpiperidin-3-yl]-2-methyl-2H-indazole-5-carboxamide (D-267) was prepared using 2-methyl-2H-indazole-5-carboxylic acid. MS found for C24H28N10O2 as (M+H)⁺ 489.4.

¹H NMR (500 MHz, DMSO) δ 10.86 (s, 1H), 8.55 (s, 1H), 8.43 (d, J=6.85 Hz, 1H), 8.39 (s, 1H), 8.04 (br.s., 1H), 7.77 (dd, J=8.80, 1.47 Hz, 1H), 7.70 (br. s., 1H), 7.64 (d, J=9.29 Hz, 1H), 7.58 (s, 1H), 7.47 (s, 1H), 7.28 (br. s., 1H), 5.54-5.06 (m, 1H), 4.20 (s, 3H), 4.15-4.02 (m, 2H), 3.74 (s, 3H), 3.10 (t, J=12.47 Hz, 1H), 2.07-1.56 (m, 4H), 1.12 (d, J=6.85 Hz, 3H).

### Example D-268: Synthesis of 1-N-[(2R,3R)-1-{5-carbamoyl-6-[(1-methyl-1H-pyrazol-4-yl)amino]pyrazin-2-yl}-2-methylpiperidin-3-yl]benzene-1,4-dicarboxamide (D-268)

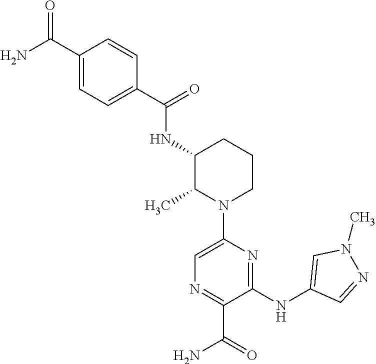 [SMILES: C[C@@H]1[C@H](NC(=O)c2ccc(C(N)=O)cc2)CCCN1c1cnc(C(N)=O)c(Nc2cnn(C)c2)n1]

In a similar manner as described in Example 8, 1-N-[(2R,3R)-1-{5-carbamoyl-6-[(1-methyl-1H-pyrazol-4-yl)amino]pyrazin-2-yl}-2-methylpiperidin-3-yl]benzene-1,4-dicarboxamide (D-268) was prepared using 4-carbamoylbenzoic acid. MS found for C23H27N9O3 as (M+H)⁺ 478.3

¹H NMR (500 MHz, DMSO) δ 10.87 (s, 1H), 8.58 (d, J=6.59 Hz, 1H), 8.10 (s, 1H), 8.06-8.01 (m, 1H), 7.97 (s, 4H), 7.70 (br. s., 1H), 7.57 (s, 1H), 7.51 (br. s., 1H), 7.48 (s, 1H), 7.28 (br. s., 1H), 5.68-4.95 (m, 1H), 4.27-3.97 (m, 2H), 3.75 (s, 3H), 3.09 (t, J=12.08 Hz, 1H), 2.08-1.54 (m, 4H), 1.11 (d, J=6.86 Hz, 3H).

### Example D-269: Synthesis of 5-[(2R,3R)-3-(4-cyclopropylbenzamido)-2-methylpiperidin-1-yl]-3-[(3-{[(2-methoxyethyl)(methyl)amino]methyl}-1,2-thiazol-5-yl)amino]pyrazine-2-carboxamide (D-269)

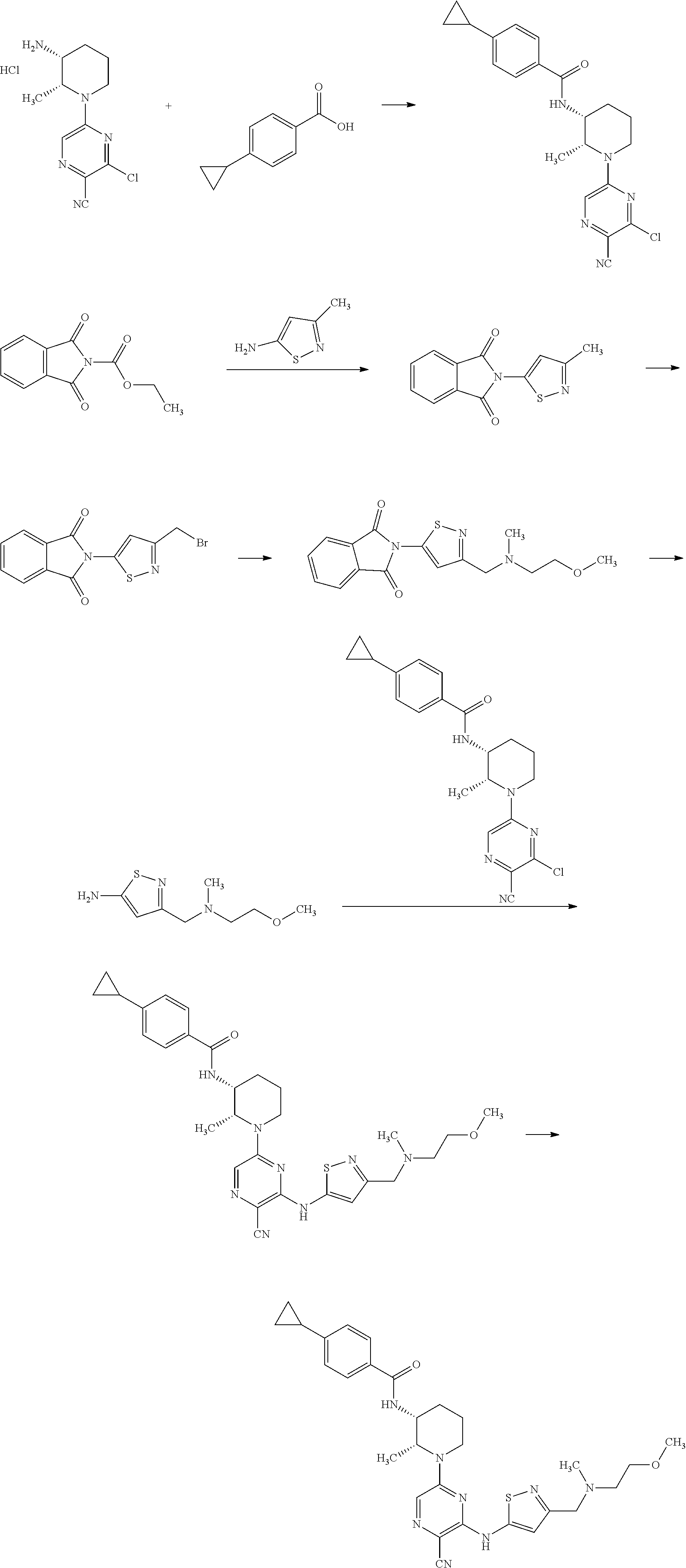

5-[(2R,3R)-3-amino-2-methylpiperidin-1-yl]-3-chloropyrazine-2-carbonitrile hydrochloride (850 mg, 2.95 mmol) and 4-cyclopropylbenzoic acid (718 mg, 4.42 mmol) were dissolved in DMF (20 mL), then DIPEA (2.6 mL, 14.7 mmol) and PyBOP (2.36 g, 4.42 mmol) were added and the mixture was stirred at room temperature 3 h. Water and DCM were added and the mixture was extracted with DCM. The crude obtained was purified by silica flash chromatography with 30% to 50% ethyl acetate in cyclohexane to afford N-[(2R,3R)-1-(6-chloro-5-cyanopyrazin-2-yl)-2-methylpiperidin-3-yl]-4-cyclopropylbenzamide (623.8 mg, 58% yield) as a yellow solid. MS found for C21H22ClN5O as (M+H)⁺ 396.4.

3-Methyl-1,2-thiazol-5-amine hydrochloride (6.002 g, 39.8 mmol) was eluted on a SCX cartridge. The residue obtained was dissolved in toluene (100 mL), then N-carbethoxyphthalimide (8.3 g, 37.8 mmol) was added and the mixture was heated at 110° C. and stirred overnight. Toluene was evaporated, then the residue was diluted with DCM and HCl 1 M and extracted with DCM. Organic layer was evaporated to give a crude which was purified by silica flash chromatography with 20% to 100% ethyl acetate in cyclohexane to afford 2-(3-methyl-1,2-thiazol-5-yl)-2,3-dihydro-1H-isoindole-1,3-dione (6.38 g, 66% yield). MS found for C12H8N2O2S as (M+H)⁺ 244.9.

2-(3-methyl-1,2-thiazol-5-yl)-2,3-dihydro-1H-isoindole-1,3-dione (6.38 g, 26.1 mmol) was dissolved in DCE (100 mL), then NBS (6.001 g, 34 mmol) and AIBN (862 mg, 5.2 mmol) were added and the mixture was stirred at 90° C. for 6 h. After cooling, a saturated solution of Na₂S₂O₅ was added and the layer was extracted with DCM. The collected organic phases were dried over Na₂SO₄, filtered and evaporated to give a crude which was purified by silica flash chromatography with 50% to 100% DCM in cyclohexane, then 100% ethyl acetate, to afford 2-[3-(bromomethyl)-1,2-thiazol-5-yl]-2,3-dihydro-1H-isoindole-1,3-dione (4.1 g, 49% yield) as a pale yellow solid. MS found for C12H7BrN2O2S as (M+H)⁺ 324.9.

2-[3-(Bromomethyl)-1,2-thiazol-5-yl]-2,3-dihydro-1H-isoindole-1,3-dione (600 mg, 1.86 mmol) was dissolved in DMF (9 mL), then DIPEA (0.65 mL, 3.71 mmol) and (2-methoxyethyl)methyl)amine (0.24 mL, 2.23 mmol) were added and the mixture was stirred at room temperature for 1 h. The solvent was evaporated to give a crude which was purified by silica flash chromatography with 50% to 100% ethyl acetate in cyclohexane, to afford 2-(3-{[(2-methoxyethyl)(methyl)amino]methyl}-1,2-thiazol-5-yl)-2,3-dihydro-1H-isoindole-1,3-dione (134 mg, 22% yield) as a yellow oil. MS found for C16H17N3O3S as (M+H)⁺ 332.0. 2-(3-{[(2-Methoxyethyl)(methyl)amino]methyl}-1,2-thiazol-5-yl)-2,3-dihydro-1H-isoindole-1,3-dione (134 mg, 0.4 mmol) was dissolved in EtOH (4 mL), then hydrazine (0.075 mL, 1.01 mmol) was added and the mixture was stirred at 40° C. overnight. The solvent was evaporated, then the mixture was purified by SCX to obtain 3-{[(2-methoxyethyl)(methyl)amino]methyl}-1,2-thiazol-5-amine (83.8 mg, quant. yield) as a colorless oil. MS found for C8H15N3OS as (M+H)⁺ 202.1.

A mixture of N-[(2R,3R)-1-(6-chloro-5-cyanopyrazin-2-yl)-2-methylpiperidin-3-yl]-4-cyclopropylbenzamide (137 mg, 0.35 mmol), 3-{[(2-methoxyethyl)(methyl)amino]methyl}-1,2-thiazol-5-amine (83.8 mg, 0.42 mmol), Pd(OAc)₂ (16 mg, 0.069 mmol), (+/−) BINAP (46 mg, 0.069 mmol), fine powder Cs₂CO₃ (0.541 g, 1.66 mmol) in dioxane (6 mL) was degassed with a nitrogen stream for 10 min. The mixture was stirred in a nitrogen atmosphere at 100° C. for 2 h, then cooled to room temperature, diluted with ethyl acetate, filtered through celite, and concentrated in vacuo. The residue was purified by silica NH flash chromatography with 50 to 100% ethyl acetate in cyclohexane to isolate N-[(2R,3R)-1-{5-cyano-6-[(3-{[(2-methoxyethyl)(methyl)amino]methyl}-1,2-thiazol-5-yl)amino]pyrazin-2-yl}-2-methylpiperidin-3-yl]-4-cyclopropylbenzamide (53 mg, 27% yield). MS found for C29H36N8O2S as (M+H)⁺ 561.2.

To a solution of N-[(2R,3R)-1-{5-cyano-6-[(3-{[(2-methoxyethyl)(methyl)amino]methyl}-1,2-thiazol-5-yl)amino]pyrazin-2-yl}-2-methylpiperidin-3-yl]-4-cyclopropylbenzamide (53 mg, 0.095 mmol) in MeOH (2 mL) and DMSO (0.2 mL) were added TEA (0.5 mL), solid NaOH (9 mg) and 30% H₂O₂ (0.05 mL). The mixture was stirred at room temperature 1 h, then at 60° C. 6 h. Water and DCM were added and the mixture was extracted with DCM. The organic phase was dried over Na₂SO₄, filtered and evaporated to give a crude which was purified by silica NH flash chromatography with 60 to 100% ethyl acetate in cyclohexane to afford 5-[(2R,3R)-3-(4-cyclopropylbenzamido)-2-methylpiperidin-1-yl]-3-[(3-{[(2-methoxyethyl)(methyl)amino]methyl}-1,2-thiazol-5-yl)amino]pyrazine-2-carboxamide (D-269) (31.2 mg, 57% yield) as a yellow solid. MS found for C29H38N8O3S as (M+H)⁺ 579.2.

¹H NMR (500 MHz, DMSO) δ 12.34 (s, 1H), 8.35 (d, J=7.27 Hz, 1H), 7.92 (br. s., 1H), 7.83 (s, 1H), 7.79 (d, J=8.23 Hz, 2H), 7.57 (br. s., 1H), 7.17 (d, J=8.23 Hz, 2H), 6.91 (s, 1H), 5.06 (br. s., 1H), 4.46 (br. s., 1H), 4.16-3.99 (m, 1H), 3.56-3.46 (m, 2H), 3.43 (t, J=5.90 Hz, 2H), 3.23 (s, 3H), 3.17 (t, J=12.14 Hz, 1H), 2.57-2.45 (m, 2H), 2.18 (s, 3H), 2.08-1.81 (m, 3H), 1.76-1.55 (m, 2H), 1.22 (d, J=6.86 Hz, 3H), 1.05-0.98 (m, 2H), 0.77-0.70 (m, 2H).

### Example D-270: Synthesis of 5-[(2R,3R)-3-(4-cyclopropylbenzamido)-2-methylpiperidin-1-yl]-3-{[1-(2-methoxyethyl)-1H-pyrazol-4-yl]amino}pyrazine-2-carboxamide (D-270)

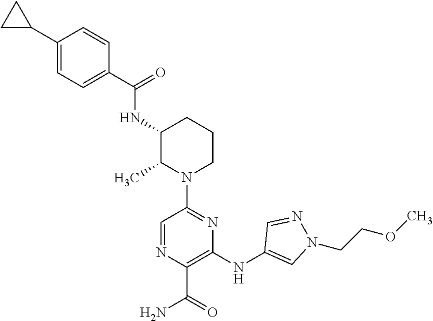 [SMILES: COCCn1cc(Nc2nc(N3CCC[C@@H](NC(=O)c4ccc(C5CC5)cc4)[C@H]3C)cnc2C(N)=O)cn1]

In a similar manner as described in Example D-269, 5-[(2R,3R)-3-(4-cyclopropylbenzamido)-2-methylpiperidin-1-yl]-3-{[1-(2-methoxyethyl)-1H-pyrazol-4-yl]amino}pyrazine-2-carboxamide (D-270) was prepared using 1-(2-methoxyethyl)-1H-pyrazol-4-amine. MS found for as C27H34N8O3 (M+H)⁺ 519.1.

¹H NMR (500 MHz, DMSO) δ 10.87 (s, 1H), 8.34 (d, J=7.04 Hz, 1H), 8.05 (s, 1H), 7.82 (d, J=8.22 Hz, 2H), 7.69 (br. s., 1H), 7.57 (s, 1H), 7.48 (s, 1H), 7.27 (br. s., 1H), 7.17 (d, J=8.41 Hz, 2H), 5.24 (br. s., 1H), 4.27-3.94 (m, 4H), 3.58-3.37 (m, 2H), 3.15-3.00 (m, 4H), 2.06-1.80 (m, 3H), 1.79-1.51 (m, 2H), 1.10 (d, J=6.85 Hz, 3H), 1.06-0.98 (m, 2H), 0.77-0.70 (m, 2H).

### Example D-271: Synthesis of 5-[(2R,3R)-3-(4-cyclopropylbenzamido)-2-methylpiperidin-1-yl]-3-[(1,5-dimethyl-1H-pyrazol-4-yl)amino]pyrazine-2-carboxamide (D-271)

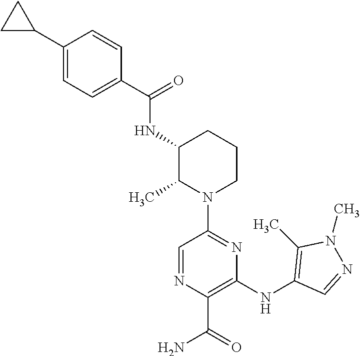 [SMILES: Cc1c(Nc2nc(N3CCC[C@@H](NC(=O)c4ccc(C5CC5)cc4)[C@H]3C)cnc2C(N)=O)cnn1C]

In a similar manner as described in Example D-269, 5-[(2R,3R)-3-(4-cyclopropylbenzamido)-2-methylpiperidin-1-yl]-3-[(1,5-dimethyl-1H-pyrazol-4-yl)amino]pyrazine-2-carboxamide (D-271) was prepared using 4-amino-1,5-dimethylpyrazole dihydrochloride. MS found for C26H32N8O2 as (M+H)⁺ 489.2.

¹H NMR (500 MHz, DMSO) δ 10.58 (s, 1H), 8.27 (d, J=7.23 Hz, 1H), 7.78 (d, J=8.33 Hz, 2H), 7.68 (br. s., 1H), 7.63 (s, 1H), 7.54 (s, 1H), 7.24 (br. s., 1H), 7.16 (d, J=8.33 Hz, 2H), 5.00-4.77 (m, 1H), 4.32-4.11 (m, 1H), 4.01 (td, J=12.39, 4.38 Hz, 1H), 3.68 (s, 3H), 3.06-2.92 (m, 1H), 2.19 (s, 3H), 2.05-1.77 (m, 3H), 1.72-1.47 (m, 2H), 1.09 (d, J=6.80 Hz, 3H), 1.04-0.97 (m, 2H), 0.77-0.69 (m, 2H).

### Example D-272: Synthesis of 5-[(2R,3R)-3-(4-cyclopropylbenzamido)-2-methylpiperidin-1-yl]-3-[(1,3-dimethyl-1H-pyrazol-4-yl)amino]pyrazine-2-carboxamide (D-272)

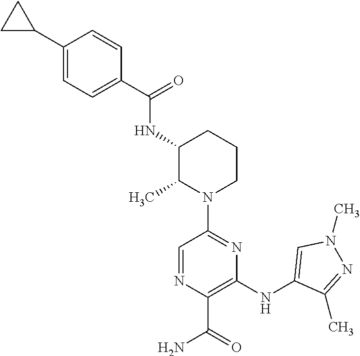 [SMILES: Cc1nn(C)cc1Nc1nc(N2CCC[C@@H](NC(=O)c3ccc(C4CC4)cc3)[C@H]2C)cnc1C(N)=O]

In a similar manner as described in Example D-269, 5-[(2R,3R)-3-(4-cyclopropylbenzamido)-2-methylpiperidin-1-yl]-3-[(1,3-dimethyl-1H-pyrazol-4-yl)amino]pyrazine-2-carboxamide (D-272) was prepared using 1,3-dimethyl-1H-pyrazol-4-amine hydrochloride. MS found for C26H32N8O2 as (M+H)⁺ 489.2.

¹H NMR (500 MHz, DMSO) δ 10.90 (s, 1H), 8.33 (d, J=6.58 Hz, 1H), 8.01 (s, 1H), 7.82 (d, J=8.11 Hz, 2H), 7.69 (br. s., 1H), 7.55 (s, 1H), 7.25 (br. s., 1H), 7.17 (d, J=8.33 Hz, 2H), 5.57-5.20 (m, 1H), 4.21-3.94 (m, 2H), 3.69 (s, 3H), 3.08 (t, J=12.39 Hz, 1H), 2.14 (s, 3H), 1.80-2.05 (m, 3H), 1.77-1.49 (m, 2H), 1.06 (d, J=6.80 Hz, 3H), 1.03-0.96 (m, 2H), 0.80-0.67 (m, 2H).

### Example D-273: Synthesis of 5-[(2R,3R)-3-(4-cyclopropylbenzamido)-2-methylpiperidin-1-yl]-3-[(1-ethyl-1H-pyrazol-4-yl)amino]pyrazine-2-carboxamide (D-273)

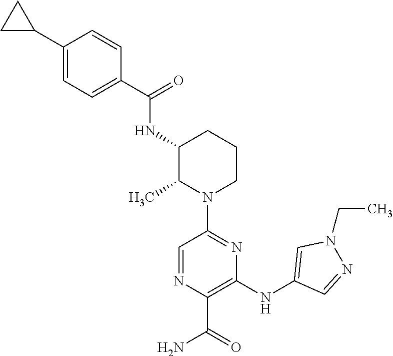 [SMILES: CCn1cc(Nc2nc(N3CCC[C@@H](NC(=O)c4ccc(C5CC5)cc4)[C@H]3C)cnc2C(N)=O)cn1]

In a similar manner as described in Example D-269, 5-[(2R,3R)-3-(4-cyclopropylbenzamido)-2-methylpiperidin-1-yl]-3-[(1-ethyl-1H-pyrazol-4-yl)amino]pyrazine-2-carboxamide (D-273) was prepared using 1-ethyl-1H-pyrazol-4-ylamine. MS found for C26H32N8O2 as (M+H)⁺ 489.2.

¹H NMR (500 MHz, DMSO) δ 10.85 (s, 1H), 8.36 (d, J=6.85 Hz, 1H), 8.04 (s, 1H), 7.81 (d, J=8.22 Hz, 2H), 7.69 (s, 1H), 7.68 (br. s., 1H), 7.57 (s, 1H), 7.45 (s, 1H), 7.27 (d, J=1.96 Hz, 1H), 7.18 (d, J=8.41 Hz, 2H), 5.29 (br. s., 1H), 4.22-3.88 (m, 4H), 3.17-2.98 (m, 1H), 2.05-1.79 (m, 3H), 1.74-1.52 (m, 2H), 1.23-1.06 (m, 6H), 1.05-0.96 (m, 2H), 0.79-0.63 (m, 2H).

### Example D-274: Synthesis of 5-[(2R,3R)-3-(4-cyclopropylbenzamido)-2-methylpiperidin-1-yl]-3-{[1-(oxan-4-yl)-1H-pyrazol-4-yl]amino}pyrazine-2-carboxamide (D-274)

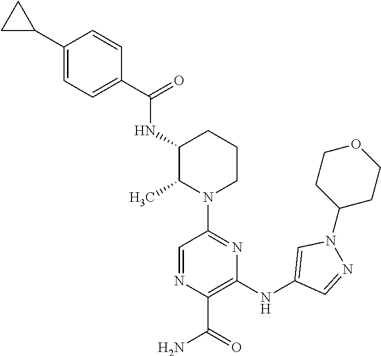 [SMILES: C[C@@H]1[C@H](NC(=O)c2ccc(C3CC3)cc2)CCCN1c1cnc(C(N)=O)c(Nc2cnn(C3CCOCC3)c2)n1]

In a similar manner as described in Example D-269, 5-[(2R,3R)-3-(4-cyclopropylbenzamido)-2-methylpiperidin-1-yl]-3-{[1-(oxan-4-yl)-1H-pyrazol-4-yl]amino}pyrazine-2-carboxamide (D-274) was prepared using 1-tetrahydro-2H-pyran-4-yl-1H-pyrazol-4-amine. MS found for C29H36N8O3 as (M+H)⁺ 545.1.

¹H NMR (500 MHz, DMSO) δ 10.86 (s, 1H), 8.37 (d, J=6.94 Hz, 1H), 8.02 (s, 1H), 7.81 (d, J=8.31 Hz, 2H), 7.68 (br. s., 1H), 7.59 (s, 1H), 7.49 (s, 1H), 7.27 (br. s., 1H), 7.18 (d, J=8.22 Hz, 2H), 5.22 (br. s., 1H), 4.32-3.95 (m, 3H), 3.73 (d, J=10.37 Hz, 1H), 3.49 (br. s., 1H), 3.22-3.02 (m, 2H), 2.86 (br. s., 1H), 1.14 (d, J=6.94 Hz, 3H), 1.08-0.92 (m, 2H), 0.80-0.66 (m, 2H).

### Example D-275: Synthesis of 5-[(2R,3R)-3-(4-cyclopropylbenzamido)-2-methylpiperidin-1-yl]-3-{[1-(difluoromethyl)-1H-pyrazol-4-yl]amino}pyrazine-2-carboxamide (D-275)

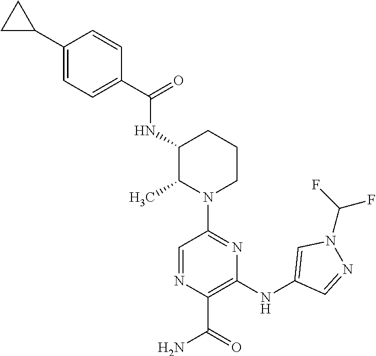 [SMILES: C[C@@H]1[C@H](NC(=O)c2ccc(C3CC3)cc2)CCCN1c1cnc(C(N)=O)c(Nc2cnn(C(F)F)c2)n1]

In a similar manner as described in Example D-269, 5-[(2R,3R)-3-(4-cyclopropylbenzamido)-2-methylpiperidin-1-yl 1]-3-{[1-(difluoromethyl)-1H-pyrazol-4-yl]amino}pyrazine-2-carboxamide (D-275) was prepared using 1-(difluoromethyl)-1H-pyrazol-4-amine hydrochloride. MS found for C25H28F2N8O2 as (M+H)⁺ 511.0.

¹H NMR (500 MHz, DMSO) δ 11.05 (s, 1H), 8.48 (br. s., 1H), 8.38 (d, J=6.72 Hz, 1H), 7.95 (s, 1H), 7.81-7.73 (m, 3H), 7.92-7.66 (m, 1H), 7.66 (s, 1H), 7.36 (br. s., 1H), 7.18 (d, J=8.23 Hz, 2H), 5.27 (br. s., 1H), 4.26-3.93 (m, 2H), 3.19-3.03 (m, 1H), 2.06-1.90 (m, 2H), 1.86 (d, J=13.17 Hz, 1H), 1.76-1.67 (m, 1H), 1.67-1.54 (m, 1H), 1.11 (d, J=6.86 Hz, 3H), 1.05-0.98 (m, 2H), 0.78-0.71 (m, 2H).

### Example D-276: Synthesis of 5-[(2R,3R)-3-(4-cyclopropylbenzamido)-2-methylpiperidin-1-yl]-3-{[1-methyl-5-(trifluoromethyl)-1H-pyrazol-4-yl]amino}pyrazine-2-carboxamide (D-276)

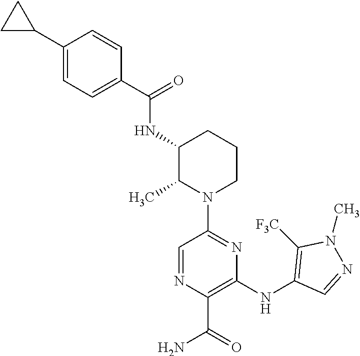 [SMILES: C[C@@H]1[C@H](NC(=O)c2ccc(C3CC3)cc2)CCCN1c1cnc(C(N)=O)c(Nc2cnn(C)c2C(F)(F)F)n1]

In a similar manner as described in Example D-269, 5-[(2R,3R)-3-(4-cyclopropylbenzamido)-2-methylpiperidin-1-yl]-3-{[1-methyl-5-(trifluoromethyl)-1H-pyrazol-4-yl]amino}pyrazine-2-carboxamide (D-276) was prepared using 1-methyl-5-(trifluoromethyl)-1H-pyrazol-4-amine hydrochloride. MS found for C26H29F3N8O2 as (M+H)⁺ 543.1.

¹H NMR (500 MHz, DMSO) δ 11.42 (s, 1H), 8.32 (d, J=7.04 Hz, 1H), 8.14 (s, 1H), 7.83-7.74 (m, 3H), 7.70 (s, 1H), 7.36 (br. s., 1H), 7.17 (d, J=8.22 Hz, 2H), 5.13-4.81 (m, 1H), 4.36-4.13 (m, 1H), 4.03 (br. s., 1H), 3.91 (s, 3H), 3.08 (t, J=12.33 Hz, 1H), 2.06-1.79 (m, 3H), 1.72-1.49 (m, 2H), 1.14 (d, J=7.04 Hz, 3H), 1.07-0.93 (m, 2H), 0.79-0.66 (m, 2H).

### Example D-277: Synthesis of 3-[(2R,3R)-3-(4-cyclopropylbenzamido)-2-methylpiperidin-1-yl]-5-[(1-methyl-1H-pyrazol-4-yl)amino]-1,2,4-triazine-6-carboxamide (D-277)

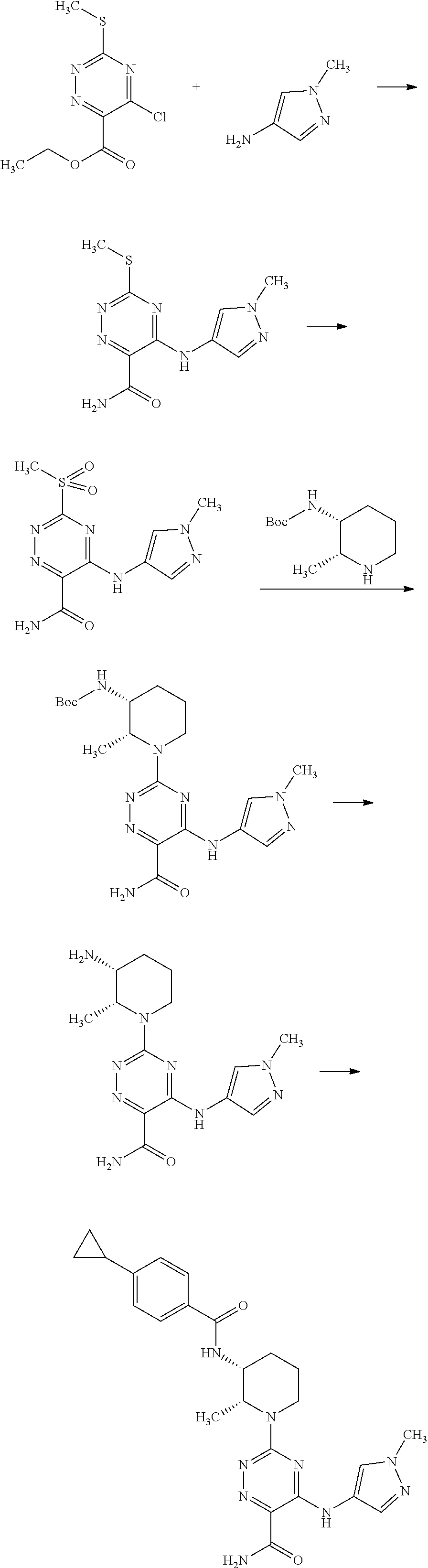

Ethyl 5-chloro-3-(methylsulfanyl)-1,2,4-triazine-6-carboxylate (2.5 g, 10.7 mmol) was dissolved in MeCN, then 1-methylpyrazol-4-amine (1.56 g, 16 mmol) was added, followed by DIPEA (2.8 mL, 16 mmol). The mixture was stirred at room temperature 2 h, then 120 mL of NH₃ 7 M in MeOH were added and the mixture was stirred at room temperature 4 h. The solid precipitated was washed with few MeCN and then with cyclohexane, dried in a oven at 50° C. overnight to obtain 5-[(1-methylpyrazol-4-yl)amino]-3-(methylsulfanyl)-1,2,4-triazine-6-carboxamide (2.4812 g, 87% yield) as a green solid. MS found for C9H11N7OS as (M+H)⁺ 266.0. 5-[(1-methylpyrazol-4-yl)amino]-3-(methylsulfanyl)-1,2,4-triazine-6-carboxamide (618 mg, 2.33 mmol) was suspended in NMP (20 mL), then mCPBA (1.56 g, 6.99 mmol) was added and the mixture was stirred at room temperature 2 h. Ethyl acetate and a mixture 1:1 of NaHCO₃ saturated solution and Na₂S₂O₃ saturated solution were added. The aqueous phase was extracted with ethyl acetate, then the organic layer was dried over Na₂SO₄, filtered and evaporated to afford a crude which was purified by silica flash chromatography with 60% to 100% ethyl acetate in cyclohexane, to obtain 3-methanesulfonyl-5-[(1-methyl-1H-pyrazol-4-yl)amino]-1,2,4-triazine-6-carboxamide in solution with NMP. MS found for C9H11N7O3S as (M+H)⁺ 297.9.

3-methanesulfonyl-5-[(1-methyl-1H-pyrazol-4-yl)amino]-1,2,4-triazine-6-carboxamide was diluted with other 5 mL of NMP, then were added DIPEA (0.6 mL, 3.49 mmol) and tert-butyl N-[(2R,3R)-2-methylpiperidin-3-yl]carbamate (0.3 g, 1.4 mmol); the mixture was stirred at 90° C. 2 h. MTBE and water were added and the mixture was extracted with MTBE. The organic phase was dried over Na₂SO₄, filtered and evaporated to afford a crude which was purified by silica flash chromatography with 70% o to 100% ethyl acetate in cyclohexane to give tert-butyl N-[(2R,3R)-1-{6-carbamoyl-5-[(1-methylpyrazol-4-yl)amino]-1,2,4-triazin-3-yl}-2-methylpiperidin-3-yl]carbamate in solution with NMP. MS found for C19H29N9O3 as (M+H)⁺ 432.1.

Tert-butyl N-[(2R,3R)-1-{6-carbamoyl-5-[(1-methylpyrazol-4-yl)amino]-1,2,4-triazin-3-yl}-2-methylpiperidin-3-yl]carbamate was dissolved in DCM (3 mL) and TFA (3 mL) was added. The mixture was stirred at room temperature 1 h, then the solvent was evaporated and the residue was passed through an SCX cartridge to afford 3-[(2R,3R)-3-amino-2-methylpiperidin-1-yl]-5-[(1-methylpyrazol-4-yl)amino]-1,2,4-triazine-6-carboxamide (73 mg) as a yellow oil. MS found for C14H21N9O as (M+H)⁺ 332.1.

3-[(2R,3R)-3-amino-2-methylpiperidin-1-yl]-5-[(1-methylpyrazol-4-yl)amino]-1,2,4-triazine-6-carboxamide (73 mg, 0.22 mmol) and 4-cyclopropylbenzoic acid (40 mg, 0.25 mmol) were dissolved in DMF (2 mL), then DIPEA (0.15 mL, 0.82 mmol) and PyBOP (0.128 g, 0.25 mmol) were added and the mixture was stirred at room temperature 2 h. Water and DCM were added and the mixture was extracted with DCM. The crude obtained was purified by SCX, then by silica flash chromatography with 60% to 100% ethyl acetate in cyclohexane to afford 3-[(2R,3R)-3-(4-cyclopropylbenzamido)-2-methylpiperidin-1-yl]-5-[(1-methyl-1H-pyrazol-4-yl)amino]-1,2,4-triazine-6-carboxamide (D-277) (34.4 mg, 44% yield) as a yellow solid. MS found for C24H29N9O2 as (M+H)⁺ 476.1.

¹H NMR (500 MHz, DMSO) δ 11.13-10.86 (m, 1H), 8.29 (br. s., 3H), 7.82 (br. s., 2H), 7.68 (br. s., 1H), 7.63-7.46 (m, 1H), 7.18 (d, J=7.68 Hz, 2H), 5.69-5.35 (m, 1H), 5.05-4.27 (m, 1H), 4.15-3.92 (m, 1H), 3.84 (s, 3H), 3.06 (t, J=12.49 Hz, 1H), 2.09-1.47 (m, 5H), 1.10 (d, J=6.59 Hz, 3H), 1.01 (dd, J=8.37, 2.06 Hz, 2H), 0.74 (d, J=3.57 Hz, 2H).

### Example D-278: Synthesis of 5-[(2R,3R)-3-(4-cyclopropylbenzamido)-2-methylpiperidin-1-yl]-3-{[1-methyl-3-(trifluoromethyl)-1H-pyrazol-4-yl]amino}pyrazine-2-carboxamide (D-278)

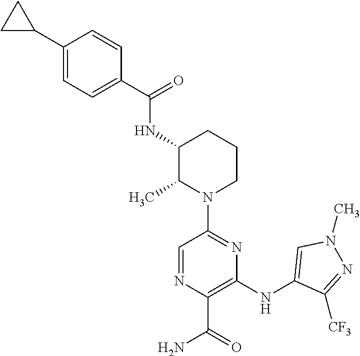 [SMILES: C[C@@H]1[C@H](NC(=O)c2ccc(C3CC3)cc2)CCCN1c1cnc(C(N)=O)c(Nc2cn(C)nc2C(F)(F)F)n1]

In a similar manner as described in Example D-269, 5-[(2R,3R)-3-(4-cyclopropylbenzamido)-2-methylpiperidin-1-yl]-3-{[1-methyl-3-(trifluoromethyl)-1H-pyrazol-4-yl]amino}pyrazine-2-carboxamide (D-278) was prepared using 1-methyl-3-(trifluoromethyl)-1H-pyrazol-4-amine. MS found for C26H29F3N8O2 as (M+H)⁺ 543.1.

¹H NMR (500 MHz, DMSO) δ 11.49 (s, 1H), 8.42-8.32 (m, 2H), 7.82 (d, J=8.23 Hz, 2H), 7.76 (br. s., 1H), 7.67 (s, 1H), 7.34 (br. s., 1H), 7.18 (d, J=8.37 Hz, 2H), 5.45 (br. s., 1H), 4.23-3.95 (m, 2H), 3.88 (s, 3H), 3.12 (t, J=12.14 Hz, 1H), 2.04-1.91 (m, 2H), 1.86 (d, J=13.04 Hz, 1H), 1.78-1.67 (m, 1H), 1.67-1.51 (m, 1H), 1.08 (d, J=6.86 Hz, 3H), 1.05-0.98 (m, 2H), 0.79-0.69 (m, 2H).

### Example D-279: Synthesis of 3-[(1-cyclopropyl-1H-pyrazol-4-yl)amino]-5-[(2R,3R)-3-(4-cyclopropylbenzamido)-2-methylpiperidin-1-yl]pyrazine-2-carboxamide (D-279)

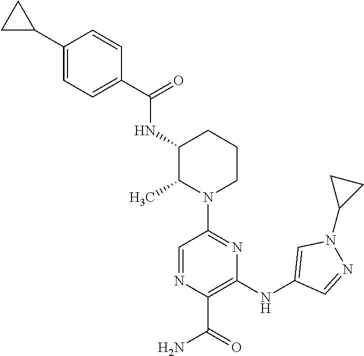 [SMILES: C[C@@H]1[C@H](NC(=O)c2ccc(C3CC3)cc2)CCCN1c1cnc(C(N)=O)c(Nc2cnn(C3CC3)c2)n1]

In a similar manner as described in Example D-269, 3-[(1-cyclopropyl-1H-pyrazol-4-yl)amino]-5-[(2R,3R)-3-(4-cyclopropylbenzamido)-2-methylpiperidin-1-yl]pyrazine-2-carboxamide (D-279) was prepared using 1-cyclopropyl-1H-pyrazol-4-amine. MS found for C27H32N8O2 as (M+H)⁺ 501.2.

¹H NMR (500 MHz, DMSO) δ 10.85 (s, 1H), 8.37 (d, J=7.24 Hz, 1H), 8.00 (s, 1H), 7.80 (d, J=8.33 Hz, 2H), 7.69 (br. s., 1H), 7.59 (s, 1H), 7.47 (s, 1H), 7.28 (br. s., 1H), 7.16 (d, J=8.33 Hz, 2H), 5.15 (br. s., 1H), 4.31-3.90 (m, 2H), 3.62 (tt, J=7.40, 3.78 Hz, 1H), 3.15-2.98 (m, 1H), 2.10-1.80 (m, 3H), 1.76-1.52 (m, 2H), 1.13 (d, J=7.02 Hz, 3H), 1.06-0.97 (m, 2H), 0.95-0.78 (m, 2H), 0.76-0.40 (m, 4H).

### Example D-280: Synthesis of 5-[(2R,3R)-3-(4-cyclopropylbenzamido)-2-methylpiperidin-1-yl]-3-{[1-(piperidin-4-yl)-1H-pyrazol-4-yl]amino}pyrazine-2-carboxamide (D-280)

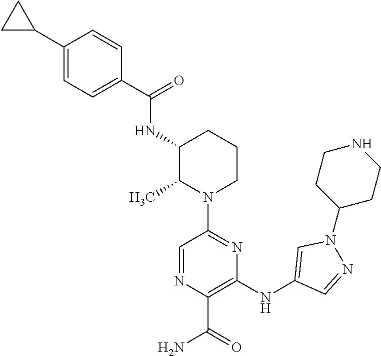 [SMILES: C[C@@H]1[C@H](NC(=O)c2ccc(C3CC3)cc2)CCCN1c1cnc(C(N)=O)c(Nc2cnn(C3CCNCC3)c2)n1]

In a similar manner as described in Example D-269, 5-[(2R,3R)-3-(4-cyclopropylbenzamido)-2-methylpiperidin-1-yl]-3-{[1-(piperidin-4-yl)-1H-pyrazol-4-yl]amino}pyrazine-2-carboxamide (D-280) was prepared using tert-butyl 4-(4-amino-1H-pyrazol-1-yl)piperidine-1-carboxylate. The final product was obtained by removal of the Boc protection with TFA in DCM. MS found for C29H37N9O2 as (M+H)⁺ 544.2.

¹H NMR (500 MHz, DMSO) δ 10.86 (s, 1H), 8.34 (d, J=6.85 Hz, 1H), 7.98 (s, 1H), 7.81 (d, J=8.22 Hz, 2H), 7.68 (br. s., 1H), 7.58 (s, 1H), 7.47 (s, 1H), 7.27 (d, J=1.96 Hz, 1H), 7.17 (d, J=8.41 Hz, 2H), 5.18 (br. s., 1H), 4.27-3.93 (m, 3H), 3.09 (t, J=11.93 Hz, 1H), 2.81 (d, J=11.74 Hz, 1H), 2.72-2.55 (m, 1H), 2.43-2.26 (m, 1H), 2.13 (br. s., 1H), 2.03-1.49 (m, 10H), 1.15 (d, J=7.04 Hz, 3H), 1.05-0.97 (m, 2H), 0.77-0.70 (m, 2H).

### Example D-281: Synthesis of 5-[(2R,3R)-3-(4-cyclopropylbenzamido)-2-methylpiperidin-1-yl]-3-{[1-(2-hydroxyethyl)-1H-pyrazol-4-yl]amino}pyrazine-2-carboxamide (D-281)

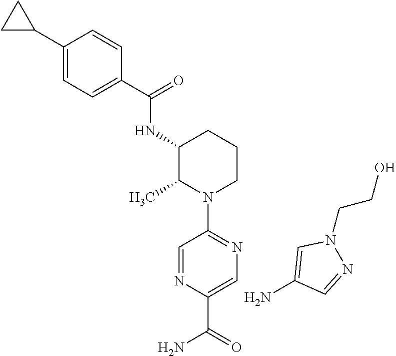

In a similar manner as described in Example D-269, 5-[(2R,3R)-3-(4-cyclopropylbenzamido)-2-methylpiperidin-1-yl]-3-{[1-(2-hydroxyethyl)-1H-pyrazol-4-yl]amino}pyrazine-2-carboxamide (D-281) was prepared using 2-(4-amino-1H-pyrazol-1-yl)ethan-1-ol. MS found for C26H32N8O3 as (M+H)⁺ 505.1

¹H NMR (500 MHz, DMSO) δ 10.87 (s, 1H), 8.34 (d, J=7.02 Hz, 1H), 8.04 (s, 1H), 7.82 (d, J=8.33 Hz, 2H), 7.69 (br. s., 1H), 7.56 (s, 1H), 7.50 (s, 1H), 7.27 (d, J=1.97 Hz, 1H), 7.17 (d, J=8.55 Hz, 2H), 5.42-5.01 (m, 1H), 4.75 (br. s., 1H), 4.31-3.94 (m, 4H), 3.59 (d, J=5.26 Hz, 2H), 3.07 (t, J=12.06 Hz, 1H), 2.05-1.80 (m, 3H), 1.74-1.49 (m, 2H), 1.10 (d, J=7.02 Hz, 3H), 1.05-0.97 (m, 2H), 0.78-0.70 (m, 2H).

### Example D-282: Synthesis of 5-[(2R,3R)-3-(4-cyclopropylbenzamido)-2-methylpiperidin-1-yl]-3-[(1H-pyrazol-4-yl)amino]pyrazine-2-carboxamide (D-282)

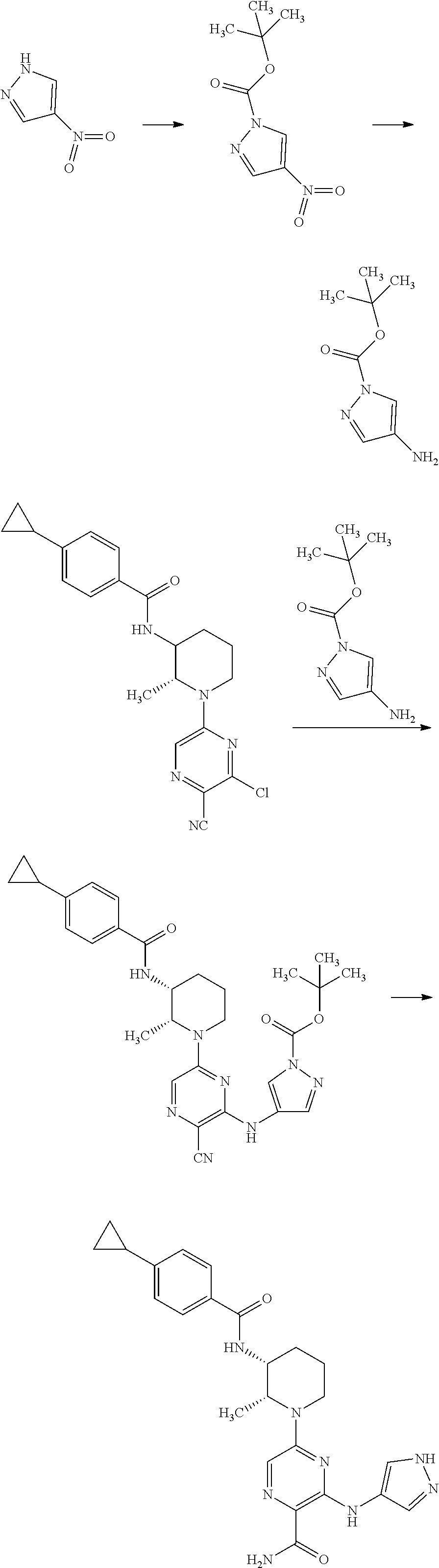

4-Nitro-1H-pyrazole (500 mg, 4.42 mmol) was dissolved in DCM (40 mL), then DMAP (54 mg, 0.44 mmol) was added, followed by Boc₂O (1.06 g, 4.86 mmol). The mixture was stirred at room temperature 2 h, then was washed with HCl 1 M and the aqueous phase was extracted with DCM. Organic layer was dried (Na₂SO₄), filtered and evaporated to give tert-butyl 4-nitro-1H-pyrazole-1-carboxylate (861.8 mg, 91% yield) as a white solid which was used in the next step without furoom temperatureher purification. Tert-butyl 4-nitro-1H-pyrazole-1-carboxylate was dissolved in 40 mL of EtOH, then 200 mg of Pd/C was added and the mixture was stirred under H₂ at ambient pressure overnight. The catalyst was filtered off and the solvent was evaporated to afford tert-butyl 4-amino-1H-pyrazole-1-carboxylate (680 mg, 92% yield) as a pale pink solid. MS found for C8H13N302 as (M+H)⁺ 184.0.

In a similar manner as described in Example D-269, 5-[(2R,3R)-3-(4-cyclopropylbenzamido)-2-methylpiperidin-1-yl]-3-[(1H-pyrazol-4-yl)amino]pyrazine-2-carboxamide (D-282) was prepared using tert-butyl 4-amino-1H-pyrazole-1-carboxylate. The final basic hydrolysis gave also the deprotection of the Boc group. MS found for C₂₄H₂₈N₈O₂ as (M+H)⁺ 461.1.

¹H NMR (500 MHz, DMSO) δ 12.53 (br. s., 1H), 10.87 (s, 1H), 8.29 (d, J=7.24 Hz, 1H), 7.95 (br. s., 1H), 7.82 (d, J=8.11 Hz, 2H), 7.69 (br. s., 1H), 7.63 (br. s., 1H), 7.56 (s, 1H), 7.27 (br. s., 1H), 7.16 (d, J=8.33 Hz, 2H), 5.17-4.85 (m, 1H), 4.38-4.14 (m, 1H), 4.09-3.96 (m, 1H), 3.06 (t, J=13.04 Hz, 1H), 2.03-1.79 (m, 3H), 1.74-1.51 (m, 2H), 1.13 (d, J=6.80 Hz, 3H), 1.01 (dd, J=8.22, 2.08 Hz, 2H), 0.78-0.70 (m, 2H).

### Example D-283: Synthesis of 3-{[1-(1-acetylpiperidin-4-yl)-1H-pyrazol-4-yl]amino}-5-[(2R,3R)-3-(4-cyclopropylbenzamido)-2-methylpiperidin-1-yl]pyrazine-2-carboxamide (D-283)

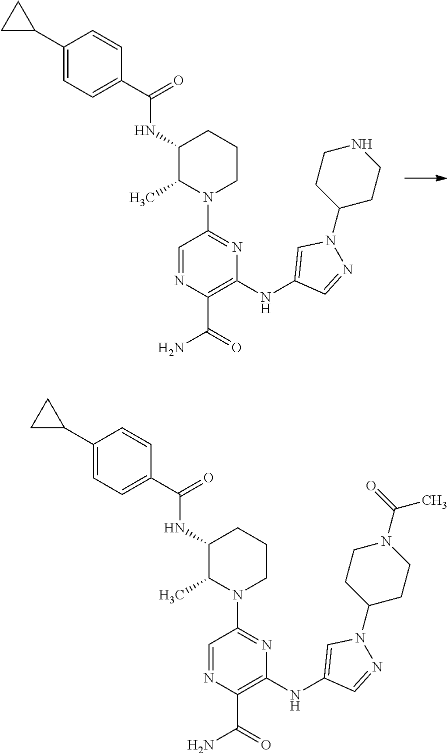 [SMILES: C[C@@H]1[C@H](NC(=O)c2ccc(C3CC3)cc2)CCCN1c1cnc(C(N)=O)c(Nc2cnn(C3CCNCC3)c2)n1 ; CC(=O)N1CCC(n2cc(Nc3nc(N4CCC[C@@H](NC(=O)c5ccc(C6CC6)cc5)[C@H]4C)cnc3C(N)=O)cn2)CC1]

5-[(2R,3R)-3-(4-cyclopropylbenzamido)-2-methylpiperidin-1-yl]-3-{[1-(piperidin-4-yl)-1H-pyrazol-4-yl]amino}pyrazine-2-carboxamide (100 mg, 0.18 mmol) was dissolved in 5 mL of dry DCM, then pyridine (0.015 mL) was added, followed by acetic anhydride (0.02 mL). The mixture was stirred at room temperature 2 h. Water was added and the two phases were separated. The aqueous was further extracted with DCM. The collected organic phases were dried over Na₂SO₄, filtered and evaporated to give a crude which was purified by silica flash chromatography with 0% to 10% methanol in DCM to afford 3-{[1-(1-acetylpiperidin-4-yl)-1H-pyrazol-4-yl]amino)}-5-[(2R,3R)-3-(4-cyclopropylbenzamido)-2-methylpiperidin-1-yl]pyrazine-2-carboxamide (D-283), (78.7 mg, 73% yield) as a pale yellow solid. MS found for C31H39N9O3 as (M+H)⁺ 586.1.

¹H NMR (500 MHz, DMSO) δ 10.91-10.78 (m, 1H), 8.39 (d, J=7.02 Hz, 1H), 8.09-7.98 (m, 1H), 7.86-7.75 (m, 2H), 7.68 (br. s., 1H), 7.58 (s, 1H), 7.50 (s, 1H), 7.27 (br. s., 1H), 7.18 (d, J=8.11 Hz, 2H), 5.23 (br. s., 1H), 4.37-3.96 (m, 3H), 4.43-3.36 (m, 2H), 3.15-3.01 (m, 1H), 2.22-1.49 (m, 12H), 2.91-1.40 (m, 2H), 1.20-1.07 (m, 3H), 1.05-0.94 (m, 2H), 0.79-0.64 (m, 2H).

### Example D-284: Synthesis of 5-[(2R,3R)-3-(4-cyclopropylbenzamido)-2-methylpiperidin-1-yl]-3-{[1-(pyridin-4-yl)-1H-pyrazol-4-yl]amino}pyrazine-2-carboxamide (D-284)

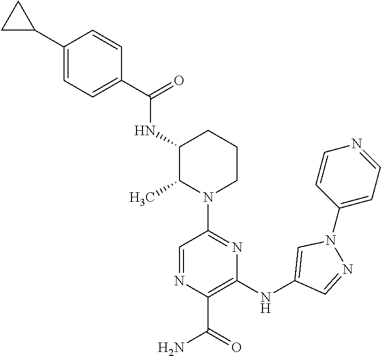 [SMILES: C[C@@H]1[C@H](NC(=O)c2ccc(C3CC3)cc2)CCCN1c1cnc(C(N)=O)c(Nc2cnn(-c3ccncc3)c2)n1]

In a similar manner as described in Example D-269, 5-[(2R,3R)-3-(4-cyclopropylbenzamido)-2-methylpiperidin-1-yl]-3-{[1-(pyridin-4-yl)-1H-pyrazol-4-yl]amino}pyrazine-2-carboxamide (D-284) was prepared using 1-pyridin-4-ylpyrazol-4-amine. MS found for C29H31N9O2 as (M+H)⁺ 538.2.

¹H NMR (500 MHz, DMSO) δ 11.19 (br. s., 1H), 8.62 (br. s., 1H), 8.42 (d, J=6.86 Hz, 1H), 8.08 (s, 3H), 7.77 (d, J=8.23 Hz, 3H), 7.69 (s, 1H), 7.55 (br. s., 2H), 7.39 (br. s., 1H), 7.17 (d, J=8.23 Hz, 2H), 5.23 (br. s., 1H), 4.21 (br. s., 1H), 4.05 (td, J=12.01, 4.80 Hz, 1H), 3.20-3.03 (m, 1H), 2.06-1.81 (m, 3H), 1.75-1.54 (m, 2H), 1.19 (d, J=7.14 Hz, 3H), 1.08-0.98 (m, 2H), 0.76 (dt, J=4.80, 2.95 Hz, 2H).

### Example D-285: Synthesis of 3-[(2R,3R)-3-(4-tert-butylbenzamido)-2-methylpiperidin-1-yl]-5-[(1-methyl-1H-pyrazol-4-yl)amino]-1,2,4-triazine-6-carboxamide (D-285)

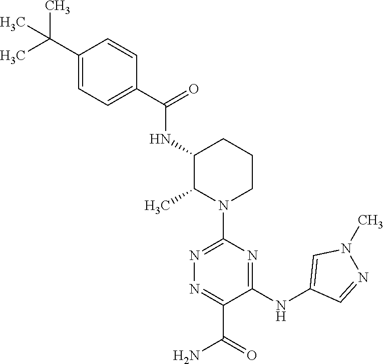 [SMILES: C[C@@H]1[C@H](NC(=O)c2ccc(C(C)(C)C)cc2)CCCN1c1nnc(C(N)=O)c(Nc2cnn(C)c2)n1]

In a similar manner as described in Example 8, 3-[(2R,3R)-3-(4-tert-butylbenzamido)-2-methylpiperidin-1-yl]-5-[(1-methyl-1H-pyrazol-4-yl)amino]-1,2,4-triazine-6-carboxamide (D-285) was prepared using 4-tert-butylbenzoic acid. MS found for C25H33N9O2 as (M+H)⁺ 492.1.

¹H NMR (500 MHz, DMSO) δ 11.25-10.78 (m, 1H), 8.44-7.92 (m, 3H), 7.91-7.77 (m, 2H), 7.74-7.55 (m, 2H), 7.51 (d, J=7.96 Hz, 2H), 5.70-5.34 (m, 1H), 4.91 (d, J=11.80 Hz, 1H), 4.16-3.92 (m, 1H), 3.87 (s, 3H), 3.06 (t, J=12.76 Hz, 1H), 2.09-1.92 (m, 1H), 1.90-1.79 (m, 1H), 1.75 (s, 1H), 1.63-1.49 (m, 1H), 1.31 (s, 9H), 1.11 (d, J=6.59 Hz, 3H).

### Example D-286: Synthesis of 3-[(2R,3R)-3-[(dimethylcarbamoyl)amino]-2-methylpiperidin-1-yl]-5-[(1-methyl-1H-pyrazol-4-yl)amino]-1,2,4-triazine-6-carboxamide (D-286)

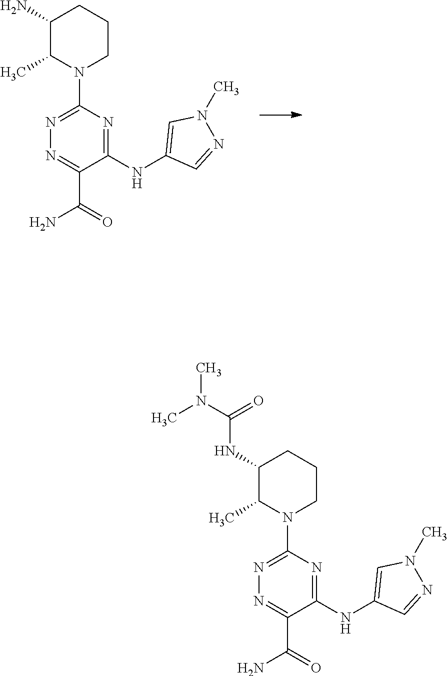

3-[(2R,3R)-3-amino-2-methylpiperidin-1-yl]-5-[(1-methylpyrazol-4-yl)amino]-1,2,4-triazine-6-carboxamide (22.5 mg, 0.068 mmol, for preparation see Example D-277) was dissolved in 2 mL of DMF, then DIPEA (0.06 mL, 0.34 mmol) and dimethylcarbamoyl chloride (0.01 mL, 0.075 mmol) were added and the mixture was stirred at room temperature for 2 h. Water and DCM were added and the mixture was extracted with DCM. The organic phase was dried over Na₂SO₄, filtered and evaporated to give a crude which was purified by silica flash chromatography with 0% to 10% methanol in DCM to afford 3-[(2R,3R)-3-[(dimethylcarbamoyl)amino]-2-methylpiperidin-1-yl]-5-[(1-methyl-1H-pyrazol-4-yl)amino]-1,2,4-triazine-6-carboxamide (D-286), (16.8 mg, 61% yield) as a yellow solid. MS found for C17H26N10O2 as (M+H)⁺ 403.4.

¹H NMR (500 MHz, DMSO) δ 10.98 (br. s., 1H), 8.42-8.13 (m, 2H), 7.74-7.41 (m, 2H), 6.09 (br. s., 1H), 5.33 (br. s., 1H), 4.86 (d, J=10.43 Hz, 1H), 3.90 (br. s., 3H), 3.66 (br. s., 1H), 3.05-2.95 (m, 1H), 2.85 (br. s., 6H), 1.90-1.73 (m, 2H), 1.70-1.39 (m, 2H), 1.05 (d, J=6.59 Hz, 3H).

### Example D-287: Synthesis of 3-{[1-(cyanomethyl)-1H-pyrazol-4-yl]amino}-5-[(2R,3R)-3-(4-cyclopropylbenzamido)-2-methylpiperidin-1-yl]pyrazine-2-carboxamide (D-287)

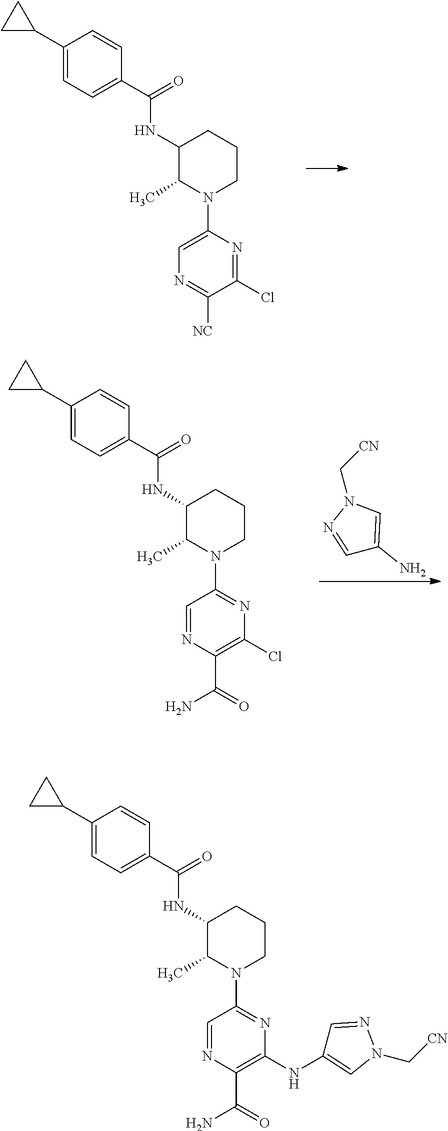 [SMILES: C[C@@H]1C(NC(=O)c2ccc(C3CC3)cc2)CCCN1c1cnc(C#N)c(Cl)n1 ; C[C@@H]1[C@H](NC(=O)c2ccc(C3CC3)cc2)CCCN1c1cnc(C(N)=O)c(Cl)n1 ; N#CCn1cc(N)cn1 ; C[C@@H]1[C@H](NC(=O)c2ccc(C3CC3)cc2)CCCN1c1cnc(C(N)=O)c(Nc2cnn(CC#N)c2)n1]

N-[(2R,3R)-1-(6-chloro-5-cyanopyrazin-2-yl)-2-methylpiperidin-3-yl]-4-cyclopropylbenzamide (120 mg, 0.3 mmol) was dissolved in 4 ml of MeOH, TEA (1 mL), DMSO (0.4 mL), NaOH (30 mg) and H₂O₂ 30% (0.2 mL) were added. The mixture was stirred at room temperature 2 h, then water and DCM were added and the mixture was extracted with DCM. The organic phase was dried over Na₂SO₄, filtered and evaporated to give a crude which was purified by silica flash chromatography with 0% to 10% methanol in DCM to afford 3-chloro-5-[(2R,3R)-3-(4-cyclopropylbenzamido)-2-methylpiperidin-1-yl]pyrazine-2-carboxamide (69.3 mg, 55% yield) as a white solid. MS found for C21H24ClN5O2 as (M+H)⁺ 414.0.

3-Chloro-5-[(2R,3R)-3-(4-cyclopropylbenzamido)-2-methylpiperidin-1-yl]pyrazine-2-carboxamide (69.3 mg, 0.17 mmol) was dissolved in dioxane (7 mL) then 2-(4-amino-1H-pyrazol-1-yl)acetonitrile (25 mg, 0.2 mmol), Pd(OAc)₂ (8 mg, 0.033 mmol), (+/−) BINAP (22 mg, 0.033 mmol) and fine powder Cs₂CO₃ (0.262 g, 0.8 mmol) were added. The mixture was stirred in a nitrogen atmosphere at 100° C. overnight, then cooled to room temperature, diluted with ethyl acetate, filtered through celite, and concentrated in vacuo. The residue was purified by silica flash chromatography with 50 to 100% ethyl acetate in cyclohexane, then it was further purified by preparative HPLC to give 3-{[1-(cyanomethyl)-1H-pyrazol-4-yl]amino}-5-[(2R,3R)-3-(4-cyclopropylbenzamido)-2-methylpiperidin-1-yl]pyrazine-2-carboxamide (D-287), (3.8 mg, 4.5% yield) as a pale yellow solid. MS found for C₂₆H₂₉N₉O₂ as (M+H)⁺ 500.2.

¹H NMR (500 MHz, DMSO) δ 10.96 (s, 1H), 8.43-8.24 (m, 2H), 7.82 (d, J=8.22 Hz, 2H), 7.75-7.64 (m, 2H), 7.60 (s, 1H), 7.36-7.27 (m, 1H), 7.18 (d, J=8.22 Hz, 2H), 5.64-5.29 (m, 3H), 4.24-3.96 (m, 2H), 3.18-3.05 (m, 1H), 2.09-1.79 (m, 3H), 1.78-1.49 (m, 2H), 1.09 (d, J=6.65 Hz, 3H), 1.02 (dd, J=8.22, 1.96 Hz, 2H), 0.75 (d, J=6.65 Hz, 2H).

### Example D-288: Synthesis of 3-{[1-(1-cyclopropanecarbonylpiperidin-4-yl)-1H-pyrazol-4-yl]amino}-5-[(2R,3R)-3-(4-cyclopropylbenzamido)-2-methylpiperidin-1-yl]pyrazine-2-carboxamide (D-288)

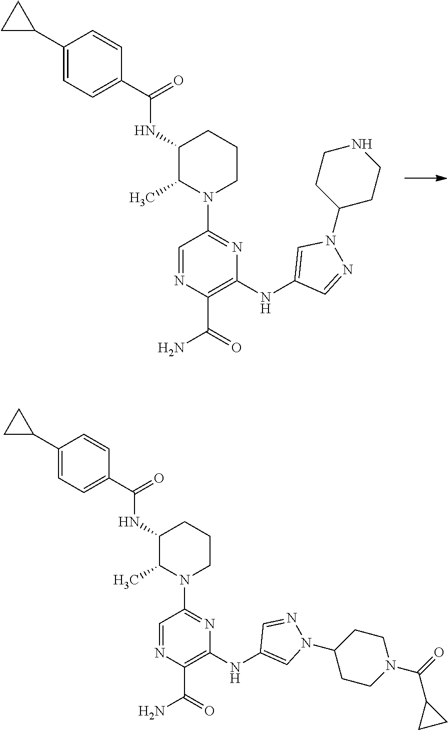 [SMILES: C[C@@H]1[C@H](NC(=O)c2ccc(C3CC3)cc2)CCCN1c1cnc(C(N)=O)c(Nc2cnn(C3CCNCC3)c2)n1 ; C[C@@H]1[C@H](NC(=O)c2ccc(C3CC3)cc2)CCCN1c1cnc(C(N)=O)c(Nc2cnn(C3CCN(C(=O)C4CC4)CC3)c2)n1]

5-[(2R,3R)-3-(4-cyclopropylbenzamido)-2-methylpiperidin-1-yl]-3-{[1-(piperidin-4-yl)-1H-pyrazol-4-yl]amino}pyrazine-2-carboxamide (26 mg, 0.048 mmol) and cyclopropanecarboxylic acid (0.006 mL, 0.072 mmol) were dissolved in 2 mL of dry DMF, then DIPEA (0.1 mL, 0.24 mmol) and PyBOP (40 mg, 0.072 mmol) were added and the mixture was stirred at room temperature 2 h. Water and DCM were added and the mixture was extracted with DCM. Organic layers were dried over Na₂SO₄, filtered and evaporated to give a crude which was purified by SCX, then by silica flash chromatography with 70% to 100% ethyl acetate in cyclohexane to afford 3-{[1-(1-cyclopropanecarbonylpiperidin-4-yl)-1H-pyrazol-4-yl]amino}-5-[(2R,3R)-3-(4-cyclopropylbenzamido)-2-methylpiperidin-1-yl]pyrazine-2-carboxamide (D-288), (19 mg, 65% yield) as a yellow solid. MS found for C33H41N9O3 as (M+H)⁺ 612.2.

¹H NMR (500 MHz, DMSO) δ 10.85 (br. s., 1H), 8.40 (d, J=6.59 Hz, 1H), 8.04 (s, 1H), 7.82 (d, J=6.31 Hz, 2H), 7.69 (br. s., 1H), 7.59 (s, 1H), 7.50 (s, 1H), 7.28 (br. s., 1H), 7.18 (d, J=8.23 Hz, 2H), 5.51-5.09 (m, 1H), 4.38-3.71 (m, 5H), 3.16-3.04 (m, 1H), 2.96-2.35 (m, 2H), 2.07-1.50 (m, 10H), 1.13 (d, J=6.86 Hz, 3H), 0.99 (d, J=8.51 Hz, 2H), 0.79-0.60 (m, 6H).

### Example D-289: Synthesis of 5-[(2R,3R)-3-(4-cyclopropylbenzamido)-2-methylpiperidin-1-yl]-3-({1-[1-(3-methyloxetane-3-carbonyl)piperidin-4-yl]-1H-pyrazol-4-yl}amino)pyrazine-2-carboxamide (D-289)

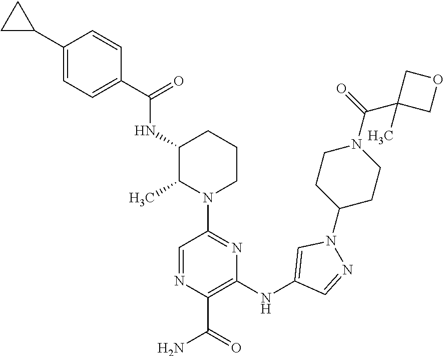 [SMILES: C[C@@H]1[C@H](NC(=O)c2ccc(C3CC3)cc2)CCCN1c1cnc(C(N)=O)c(Nc2cnn(C3CCN(C(=O)C4(C)COC4)CC3)c2)n1]

In a similar manner as described in Example D-288, 5-[(2R,3R)-3-(4-cyclopropylbenzamido)-2-methylpiperidin-1-yl]-3-({1-[1-(3-methyloxetane-3-carbonyl)piperidin-4-yl]-1H-pyrazol-4-yl}amino)pyrazine-2-carboxamide (D-289) was prepared using 3-methyloxetane-3-carboxylic acid. MS found for C34H43N9O4 as (M+H)⁺ 642.3.

¹H NMR (500 MHz, DMSO) δ 10.80-10.85 (m, 1H), 8.40 (d, J=6.6 Hz, 1H), 8.04 (s, 1H), 7.76-7.83 (m, 2H), 7.67 (br. s., 1H), 7.57 (s, 1H), 7.48 (br. s., 1H), 7.26 (br. s., 1H), 7.18 (d, J=7.7 Hz, 2H), 5.27 (br. s., 1H), 4.59-4.76 (m, 2H), 3.95-4.35 (m, 6H), 3.03-3.14 (m, 1H), 2.39-2.95 (m, 2H), 1.54-2.02 (m, 10H), 1.38-1.52 (m, 3H), 1.07-1.15 (m, 3H), 0.93-1.04 (m, 2H), 0.62-0.79 (m, 2H).

### Example D-290: Synthesis of 5-[(2R,3R)-3-(4-cyclopropylbenzamido)-2-methylpiperidin-1-yl]-3-[(5-fluoro-1-methyl-1H-pyrazol-4-yl)amino]pyrazine-2-carboxamide (D-290)

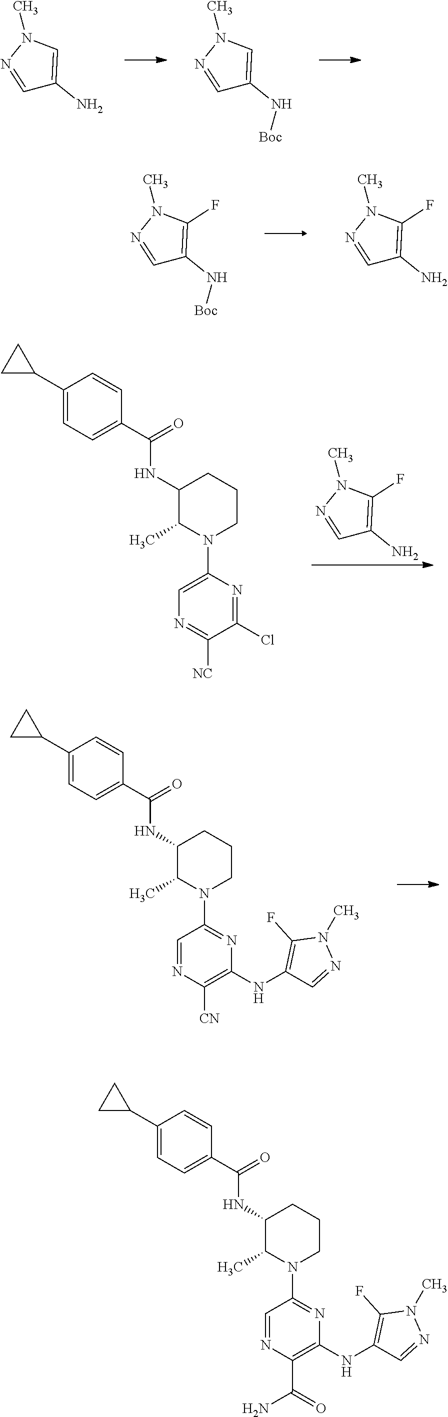

1-Methyl-1H-pyrazol-4-amine hydrochloride (350 mg, 2.62 mmol) was dissolved in DCM (10 mL), then TEA (1.45 mL, 10.48 mmol) was added, followed by Boc₂O (580 mg, 2.62 mmol) and the mixture was stirred at room temperature overnight. Water and DCM were added and the mixture was extracted with DCM. The organic phase was dried over Na₂SO₄, filtered and evaporated to give tert-butyl N-(1-methyl-1H-pyrazol-4-yl)carbamate (526 mg, quant. yield) as a purple oil. MS found for C9H15N3O2 as (M+H)⁺ 198.

Tert-butyl N-(1-methyl-1H-pyrazol-4-yl)carbamate (526 mg, 2.67 mmol) was dissolved in a mixture DMF/DCM (4 mL+4 mL), then selectfluor (950 mg, 2.67 mmol) was added and the mixture was stirred at room temperature overnight. Then, other selectfluor (190 mg) was added and the mixture was stirred at room temperature for further 2 h. Water and DCM were added and the mixture was extracted with DCM, the organic phase was dried over Na₂SO₄, filtered and evaporated to afford tert-butyl N-(5-fluoro-1-methyl-1H-pyrazol-4-yl)carbamate (640 mg) which was used in the next step without further purification. MS found for C9H14FN3O2 as (M+H)⁺ 216.0.

Tert-butyl N-(5-fluoro-1-methyl-1H-pyrazol-4-yl)carbamate (640 mg) was dissolved in DCM (10 mL) then TFA (10 mL) was added and the mixture was stirred at room temperature 2 h. The solvent was evaporated, then the mixture was passed through an SCX cartridge obtaining 5-fluoro-1-methyl-1H-pyrazol-4-amine (195 mg) as a black solid.

In a similar manner as described in Example D-269, 5-[(2R,3R)-3-(4-cyclopropylbenzamido)-2-methylpiperidin-1-yl]-3-[(5-fluoro-1-methyl-1H-pyrazol-4-yl)amino]pyrazine-2-carboxamide (D-290) was prepared using 5-fluoro-1-methyl-1H-pyrazol-4-amine. MS found for C25H29FN8O2 as (M+H)⁺ 493.1.

¹H NMR (500 MHz, DMSO) δ 10.32 (s, 1H), 8.27 (d, J=7.14 Hz, 1H), 7.78 (d, J=8.23 Hz, 2H), 7.71 (br. s., 1H), 7.62-7.57 (m, 2H), 7.30 (br. s., 1H), 7.16 (d, J=8.23 Hz, 2H), 5.07-4.75 (m, 1H), 4.32-4.08 (m, 1H), 4.04-3.92 (m, 1H), 3.64 (s, 3H), 2.98 (t, J=12.21 Hz, 1H), 2.04-1.77 (m, 3H), 1.71-1.45 (m, 2H), 1.07 (d, J=6.59 Hz, 3H), 1.03-0.95 (m, 2H), 0.77-0.68 (m, 2H).

### Example D-291: Synthesis of 5-[(2R,3R)-3-(4-tert-butylbenzamido)-2-methylpiperidin-1-yl]-3-({4-[(1-methylpiperidin-4-yl)oxy]phenyl}amino)pyrazine-2-carboxamide (D-291)

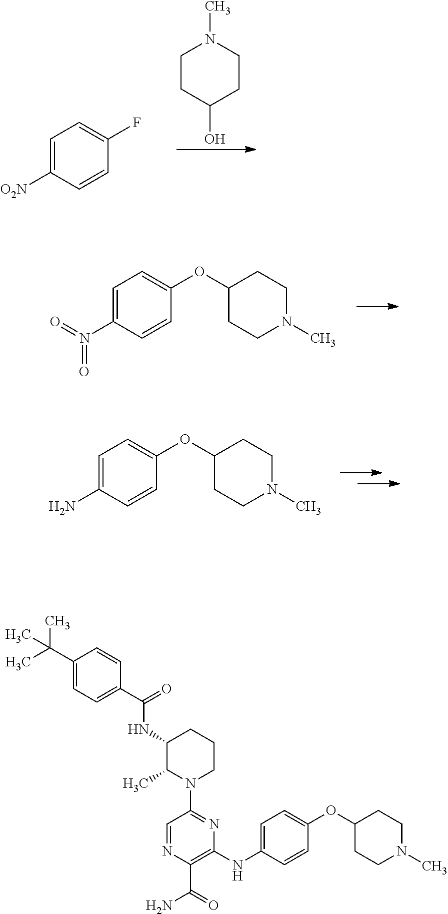

Methylpiperidin-4-ol (1 g, 8.68 mmol) and 1-fluoro-4-nitrobenzene (1 mL, 9.55 mmol) were dissolved in DMSO (40 mL), then potassium i-butoxyde (1.17 g, 10.4 mmol) was added and the mixture was stirred at room temperature overnight. Water was added and the solid that precipitated was collected by filtration, washed with water and dried to obtain 1-methyl-4-(4-nitrophenoxy)piperidine (1.4793 g, 72% yield) as a green solid. MS found for C12H16N2O3 as (M+H)⁺ 237.0.

1-Methyl-4-(4-nitrophenoxy)piperidine (1.4793 g, 6.26 mmol) was dissolved in 60 mL of EtOH, then Pd/C (209 mg) was added and the mixture was stirred under H₂ atmosphere at ambient pressure overnight. The catalyst was filtered off to afford 4-[(1-methylpiperidin-4-yl)oxy]aniline (1.2567 g, 97% yield) as a brown solid, which was used in the next step without further purification.

In a similar manner as described in Example D-216, 5-[(2R,3R)-3-(4-tert-butylbenzamido)-2-methylpiperidin-1-yl]-3-({4-[(1-methylpiperidin-4-yl)oxy]phenyl}amino)pyrazine-2-carboxamide (D-291) was prepared using 4-tert-butylbenzoic acid and 4-[(1-methylpiperidin-4-yl)oxy]aniline. MS found for C34H45N7O3 as (M+H)⁺ 600.2.

¹H NMR (500 MHz, DMSO) δ 11.09 (s, 1H), 8.33 (d, J=7.14 Hz, 1H), 7.86 (d, J=8.23 Hz, 2H), 7.73 (br. s., 1H), 7.62 (s, 1H), 7.55-7.49 (m, 4H), 7.31 (br. s., 1H), 6.86 (d, J=8.51 Hz, 2H), 5.34-4.92 (m, 1H), 4.37-3.94 (m, 3H), 3.06 (t, J=12.62 Hz, 1H), 2.86-2.61 (m, 2H), 2.47-2.03 (m, 5H), 2.01-1.79 (m, 4H), 1.74-1.54 (m, 4H), 1.32 (s, 9H), 1.08 (d, J=6.59 Hz, 3H).

### Example D-292: Synthesis of 5-[(2R,3R)-3-[4-(1-cyano-1-methylethyl)benzamido]-2-methylpiperidin-1-yl]-3-({4-[(1-methylpiperidin-4-yl)oxy]phenyl}amino)pyrazine-2-carboxamide (D-292)

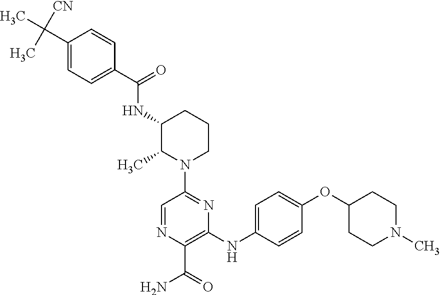 [SMILES: C[C@@H]1[C@H](NC(=O)c2ccc(C(C)(C)C#N)cc2)CCCN1c1cnc(C(N)=O)c(Nc2ccc(OC3CCN(C)CC3)cc2)n1]

In a similar manner as described in Example D-291, 5-[(2R,3R)-3-[4-(1-cyano-1-methylethyl)benzamido]-2-methylpiperidin-1-yl]-3-({4-[(1-methylpiperidin-4-yl)oxy]phenyl}amino)pyrazine-2-carboxamide (D-292) was prepared using 4-(2-cyanopropan-2-yl)benzoic acid and 4-[(1-methylpiperidin-4-yl)oxy]aniline. MS found for C34H42N8O3 as (M+H)⁺ 611.2.

¹H NMR (500 MHz, DMSO) δ 11.08 (s, 1H), 8.46 (d, J=7.34 Hz, 1H), 7.97 (d, J=8.31 Hz, 2H), 7.74 (br. s., 1H), 7.69-7.61 (m, 3H), 7.52 (d, J=8.80 Hz, 2H), 7.31 (d, J=1.47 Hz, 1H), 6.86 (d, J=8.80 Hz, 2H), 5.38-4.97 (m, 1H), 4.41-3.98 (m, 3H), 3.06 (t, J=12.47 Hz, 1H), 3.06 (t, J=12.47 Hz, 1H), 2.82-2.62 (m, 2H), 2.28 (br. s., 5H), 2.02-1.78 (m, 4H), 1.73 (s, 10H), 1.09 (d, J=6.36 Hz, 3H).

### Example D-293: Synthesis of 5-[(2R,3R)-3-(4-cyclopropylbenzamido)-2-methylpiperidin-1-yl]-3-({-[1-(2,2,2-trifluoroethyl)piperidin-4-yl]-1H-pyrazol-4-yl}amino)pyrazine-2-carboxamide (D-293)

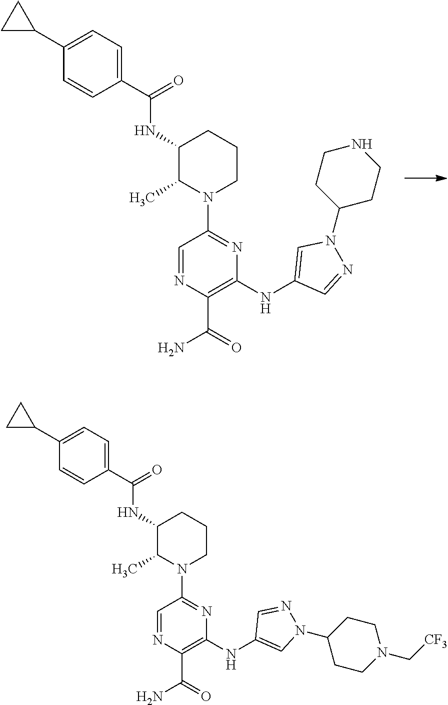 [SMILES: C[C@@H]1[C@H](NC(=O)c2ccc(C3CC3)cc2)CCCN1c1cnc(C(N)=O)c(Nc2cnn(C3CCNCC3)c2)n1 ; C[C@@H]1[C@H](NC(=O)c2ccc(C3CC3)cc2)CCCN1c1cnc(C(N)=O)c(Nc2cnn(C3CCN(CC(F)(F)F)CC3)c2)n1]

5-[(2R,3R)-3-(4-cyclopropylbenzamido)-2-methylpiperidin-1-yl]-3-{[1-(piperidin-4-yl)-1H-pyrazol-4-yl]amino}pyrazine-2-carboxamide (100.5 mg, 0.18 mmol) was dissolved in DMF (2 mL), then DIPEA (0.2 mL, 0.92 mmol) was added, followed by 2,2,2-trifluoroethyl trifluoromethanesulfonate (0.035 mL, 0.22 mmol) in 1 mL of DMF at 0° C. The mixture was then stirred at room temperature 4 h, then MeOH was added and all the organic solvents were removed by evaporation. DCM, water and brine were added and the mixture was extracted with DCM. The mixture was dried over Na₂SO₄, filtered and evaporated to afford a crude which was purified first by silica flash chromatography with 50% to 100% ethyl acetate in cyclohexane, then by preparative HPLC and then by SCX, to give 5-[(2R,3R)-3-(4-cyclopropylbenzamido)-2-methylpiperidin-1-yl]-3-({1-[1-(2,2,2-trifluoroethyl)piperidin-4-yl]-1H-pyrazol-4-yl}amino)pyrazine-2-carboxamide (16.3 mg, 24% yield) as a yellow solid. MS found for C31H38F3N9O2 as (M+H)⁺ 626.3.

¹H NMR (500 MHz, DMSO) δ 10.92 (s, 1H), 8.18 (s, 1H), 7.86 (d, J=8.31 Hz, 2H), 7.68 (d, J=6.85 Hz, 1H), 7.58-7.51 (m, 2H), 7.44 (s, 1H), 7.22 (d, J=8.31 Hz, 2H), 6.41 (br. s., 1H), 5.52 (br. s., 1H), 4.27-4.03 (m, 3H), 3.20 (td, J=13.21, 2.93 Hz, 1H), 3.05-2.86 (m, 3H), 2.69 (br. s., 1H), 2.31 (t, J=11.00 Hz, 1H), 2.04-1.66 (m, 10H), 1.25 (d, J=6.85 Hz, 3H), 1.04 (dd, J=8.31, 1.96 Hz, 2H), 0.86-0.72 (m, 2H).

### Example D-294: Synthesis of 5-[(2R,3R)-3-(6-cyclopropylpyridine-3-amido)-2-methylpiperidin-1-yl]-3-({4-[(1-methylpiperidin-4-yl)oxy]phenyl}amino)pyrazine-2-carboxamide (D-294)

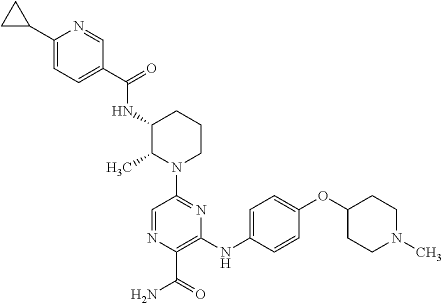 [SMILES: C[C@@H]1[C@H](NC(=O)c2ccc(C3CC3)nc2)CCCN1c1cnc(C(N)=O)c(Nc2ccc(OC3CCN(C)CC3)cc2)n1]

In a similar manner as described in Example D-291, 5-[(2R,3R)-3-(6-cyclopropylpyridine-3-amido)-2-methylpiperidin-1-yl]-3-({4-[(1-methylpiperidin-4-yl)oxy]phenyl}amino)pyrazine-2-carboxamide (D-294) was prepared using 6-cyclopropylnicotinic acid and 4-[(1-methylpiperidin-4-yl)oxy]aniline. MS found for C32H40N8O3 as (M+H)⁺ 585.3.

¹H NMR (500 MHz, DMSO) δ 11.05 (s, 1H), 8.89 (s, 1H), 8.47 (d, J=7.02 Hz, 1H), 8.11 (d, J=7.89 Hz, 1H), 7.72 (br. s., 1H), 7.61 (s, 1H), 7.50 (d, J=7.89 Hz, 2H), 7.42 (d, J=7.89 Hz, 1H), 7.30 (br. s., 1H), 6.82 (d, J=8.11 Hz, 2H), 5.33-4.90 (m, 1H), 4.10 (br. s., 3H), 3.06 (t, J=12.39 Hz, 1H), 2.26-2.13 (m, 4H), 2.13-1.12 (m, 12H), 1.11-0.96 (m, 7H).

### Example D-295: Synthesis of 3-{[4-(4-acetylpiperazin-1-yl)phenyl]amino}-5-[(2R,3R)-3-(4-cyclopropylbenzamido)-2-methylpiperidin-1-yl]pyrazine-2-carboxamide (D-295)

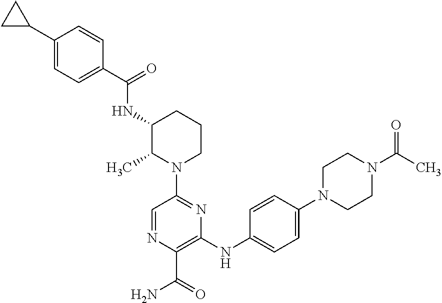 [SMILES: CC(=O)N1CCN(c2ccc(Nc3nc(N4CCC[C@@H](NC(=O)c5ccc(C6CC6)cc5)[C@H]4C)cnc3C(N)=O)cc2)CC1]

In a similar manner as described in Example D-269, 3-{[4-(4-acetylpiperazin-1-yl)phenyl]amino}-5-[(2R,3R)-3-(4-cyclopropylbenzamido)-2-methylpiperidin-1-yl]pyrazine-2-carboxamide (D-295) was prepared using 1-acetyl-4-(4-aminophenyl)piperazine. MS found for C33H40N8O3 as (M+H)⁺ 597.1.

¹H NMR (500 MHz, DMSO) δ 10.98 (br. s., 1H), 8.34 (d, J=7.34 Hz, 1H), 7.85 (d, J=8.31 Hz, 2H), 7.71 (br. s., 1H), 7.60 (s, 1H), 7.47 (d, J=9.05 Hz, 2H), 7.29 (br. s., 1H), 7.19 (d, J=8.31 Hz, 2H), 6.83 (d, J=8.31 Hz, 2H), 5.13 (br. s., 1H), 4.31-3.96 (m, 2H), 3.62-3.41 (m, 4H), 3.13-2.71 (m, 5H), 2.10-1.79 (m, 6H), 1.73-1.51 (m, 2H), 1.17-0.93 (m, 5H), 0.70-0.85 (m, 2H).

### Example D-296: Synthesis of 3-{[4-(1-acetylpiperidin-4-yl)phenyl]amino}-5-[(2R,3R)-3-(4-cyclopropylbenzamido)-2-methylpiperidin-1-yl]pyrazine-2-carboxamide (D-296)

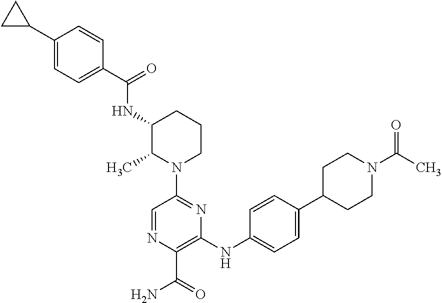 [SMILES: CC(=O)N1CCC(c2ccc(Nc3nc(N4CCC[C@@H](NC(=O)c5ccc(C6CC6)cc5)[C@H]4C)cnc3C(N)=O)cc2)CC1]

In a similar manner as described in Example D-280, 3-{[4-(1-acetylpiperidin-4-yl)phenyl]amino}-5-[(2R,3R)-3-(4-cyclopropylbenzamido)-2-methylpiperidin-1-yl]pyrazine-2-carboxamide (D-296) was prepared using 4-p-aminophenyl-1-Boc-piperidine. MS found for C34H41N7O3 as (M+H)⁺ 596.1.

¹H NMR (500 MHz, DMSO) δ 11.22 (d, J=2.74 Hz, 1H), 8.32 (d, J=7.41 Hz, 1H), 7.87-7.80 (m, 2H), 7.79-7.72 (m, 1H), 7.64 (s, 1H), 7.55 (d, J=8.51 Hz, 2H), 7.39-7.31 (m, 1H), 7.18 (dd, J=8.37, 3.43 Hz, 2H), 7.15-7.11 (m, 2H), 5.40-4.95 (m, 1H), 4.58-4.46 (m, 1H), 4.15 (br. s., 1H), 4.09-4.00 (m, 1H), 3.94-3.82 (m, 1H), 3.08 (d, J=10.43 Hz, 2H), 2.70-2.59 (m, 1H), 2.59-2.51 (m, 1H), 2.09-1.26 (m, 12H), 1.11-1.05 (m, 3H), 1.04-0.99 (m, 2H), 0.85-0.70 (m, 2H).

### Example D-297: Synthesis of 5-[(2R,3R)-3-(5-cyclopropylpyridine-2-amido)-2-methylpiperidin-1-yl]-3-({4-[(1-methylpiperidin-4-yl)oxy]phenyl}amino)pyrazine-2-carboxamide (D-297)

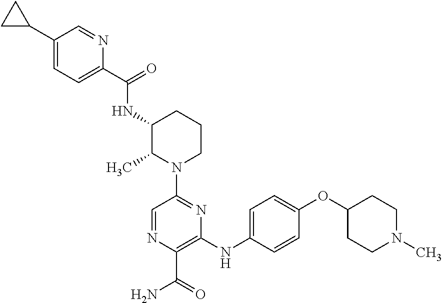 [SMILES: C[C@@H]1[C@H](NC(=O)c2ccc(C3CC3)cn2)CCCN1c1cnc(C(N)=O)c(Nc2ccc(OC3CCN(C)CC3)cc2)n1]

In a similar manner as described in Example D-291, 5-[(2R,3R)-3-(5-cyclopropylpyridine-2-amido)-2-methylpiperidindin-1-yl]-3-({4-[(1-methylpiperidin-4-yl)oxy]phenyl}amino)pyrazine-2-carboxamide (D-297) was prepared using 5-cyclopropylnicotinic acid and 4-[(1-methylpiperidin-4-yl)oxy]aniline. MS found for C32H40N8O3 as (M+H)⁺ 585.3.

¹H NMR (500 MHz, DMSO) δ 8.60-8.38 (m, 2H), 11.04 (s, 1H), 7.98 (d, J=7.96 Hz, 1H), 7.73 (br. s., 1H), 7.66-7.58 (m, 2H), 7.48 (d, J=9.06 Hz, 2H), 7.31 (br. s., 1H), 6.82 (d, J=8.78 Hz, 2H), 5.09 (br. s., 1H), 4.28-3.88 (m, 3H), 3.04 (t, J=12.35 Hz, 1H), 2.60-2.51 (m, 2H), 2.15 (s, 3H), 2.13-1.47 (m, 11H), 1.13-1.05 (m, 5H), 0.94-0.77 (m, 2H).

### Example D-298: Synthesis of 3-[(2R,3R)-3-[4-(2-hydroxypropan-2-yl)benzamido]-2-methylpiperidin-1-yl]-5-[(1-methyl-1H-pyrazol-4-yl)amino]-1,2,4-triazine-6-carboxamide (D-298)

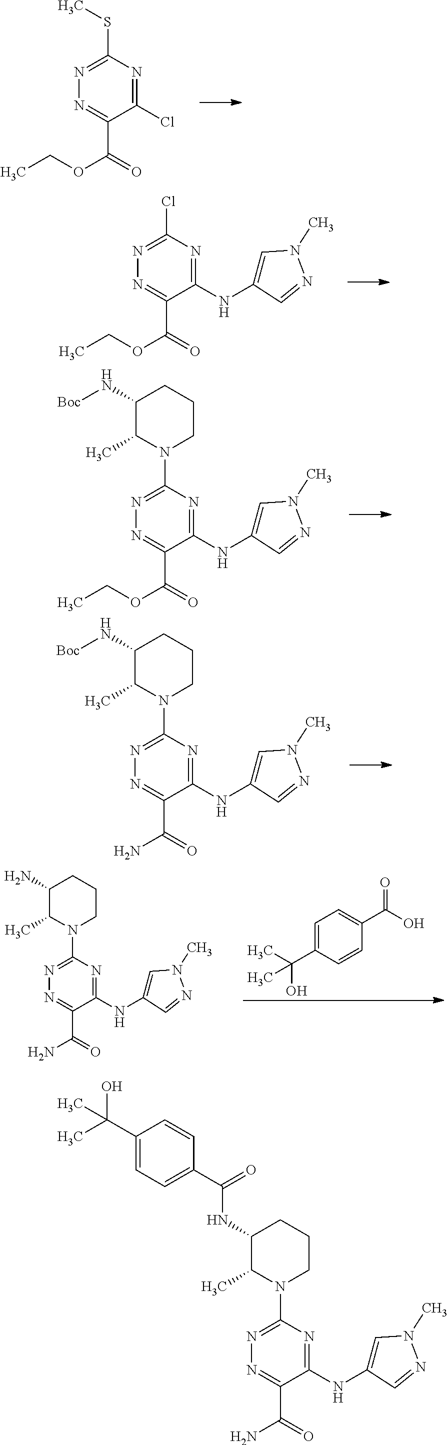

Ethyl 5-chloro-3-(methylthio)-1,2,4-triazine-6-carboxylate (1.5 g, 6.4 mmol) was dissolved in DCM (65 mL), then EtOH (0.4 mL, 6.4 mmol) was added, followed by SO₂C1₂ (3.7 mL, 45 mmol). The mixture was stirred at room temperature 2 h, then DIPEA (22.4 mL, 18.4 mmol) was slowly added at 0° C., followed by 1-methyl-1H-pyrazol-4-amine (624 mg, 6.4 mmol). The mixture was stirred at room temperature 2 h, then water and DCM were added and the mixture was extracted with DCM (3×100 mL). The collected organic phases were dried over Na₂SO₄, filtered and evaporated to afford a crude which was purified by silica flash chromatography with 40% to 70% ethyl acetate in cyclohexane, to obtain ethyl 3-chloro-5-[(1-methyl-1H-pyrazol-4-yl)amino]-1,2,4-triazine-6-carboxylate (430 mg, 24% yield) as a brown solid. MS found for C10H11ClN6O2 as (M+H)⁺ 283.0.

Ethyl 3-chloro-5-[(1-methyl-1H-pyrazol-4-yl)amino]-1,2,4-triazine-6-carboxylate (430 mg, 1.52 mmol) was dissolved in 10 mL of DMF, then tert-butyl N-[(2R,3R)-2-methylpiperidin-3-yl]carbamate (391 mg, 1.83 mmol) was added, followed by DIPEA (0.8 mL, 4.56 mmol). This mixture was stirred at 60° C. 1 h. Water and ethyl acetate were added and the mixture was extracted with ethyl acetate. The organic layer was dried over Na₂SO₄, filtered and evaporated to give a crude which was purified by silica flash chromatography with 50% to 100% ethyl acetate in cyclohexane, to afford ethyl 3-[(2R,3R)-3-{[(tert-butoxy)carbonyl]amino}-2-methylpiperidin-1-yl]-5-[(1-methyl-1H-pyrazol-4-yl)amino]-1,2,4-triazine-6-carboxylate (539.5 mg, 77% yield) as orange solid. MS found for C21H32N8O4 as (M+H)⁺ 461.1.

Ethyl 3-[(2R,3R)-3-{[(tert-butoxy)carbonyl]amino}-2-methylpiperidin-1-yl]-5-[(1-methyl-1H-pyrazol-4-yl)amino]-1,2,4-triazine-6-carboxylate (439.5 mg, 0.95 mmol) was dissolved in 10 mL of NH₃ 7 M in MeOH and stirred at room temperature 5 h. The solvent was evaporated to afford tert-butyl N-[(2R,3R)-1-{6-carbamoyl-5-[(1-methyl-1H-pyrazol-4-yl)amino]-1,2,4-triazin-3-yl}-2-methylpiperidin-3-yl]carbamate (551.8 mg, quant. yield) as orange solid, which was used in the next step without further purification. MS found for C19H29N9O3 as (M+H)⁺ 432.1. Tert-butyl N-[(2R,3R)-1-(6-carbamoyl-5-[(1-methyl-1H-pyrazol-4-yl)amino]-1,2,4-triazin-3-yl)-2-methylpiperidin-3-yl]carbamate (551.8 mg, 1.3 mmol) was dissolved in DCM (10 mL) then TFA (2 mL) was added and the mixture was stirred at room temperature 2 h. The solvent was evaporated then the residue was passed through an SCX cartridge to afford 3-[(2R,3R)-3-amino-2-methylpiperidin-1-yl]-5-[(1-methyl-1H-pyrazol-4-yl)amino]-1,2,4-triazine-6-carboxamide (403.2 mg, 95% yield) as a brown solid. MS found for C14H21N9O as (M+H)⁺ 332.1.

3-[(2R,3R)-3-amino-2-methylpiperidin-1-yl]-5-[(1-methyl-1H-pyrazol-4-yl)amino]-1,2,4-triazine-6-carboxamide (300 mg, 0.91 mmol) and 4-(2-hydroxypropan-2-yl)benzoic acid (245 mg, 1.36 mmol) were dissolved in DMF (9 mL), then PyBOP (707 mg, 1.36 mmol) was added, followed by DIPEA (0.8 mL, 4.53 mmol). The mixture was stirred at room temperature 2 h, then water and DCM were added and the mixture was extracted with DCM. The organic phase was dried over Na₂SO₄, filtered and evaporated to give a crude which purified by SCX and then by silica flash chromatography with 0% to 10% methanol in DCM, to afford 3-[(2R,3R)-3-[4-(2-hydroxypropan-2-yl)benzamido]-2-methylpiperidin-1-yl]-5-[(1-methyl-1H-pyrazol-4-yl)amino]-1,2,4-triazine-6-carboxamide (D-298), (287.4 mg, 64% yield) as a yellow solid. MS found for C24H31N9O3 as (M+H)⁺ 494.4.

¹H NMR (500 MHz, DMSO) δ 11.11-10.88 (m, 1H), 8.48-7.93 (m, 3H), 7.91-7.77 (m, 2H), 7.68 (s, 1H), 7.64-7.47 (m, 3H), 5.70-5.35 (m, 1H), 5.13 (s, 1H), 5.00-4.25 (m, 1H), 4.15-3.92 (m, 1H), 3.86 (s, 3H), 3.07 (t, J=12.13 Hz, 1H), 1.76 (br. s., 4H), 1.45 (s, 6H), 1.12 (d, J=6.26 Hz, 3H).

### Example D-299: Synthesis of 5-[(2R,3R)-3-(4-cyclopropylbenzamido)-2-methylpiperidin-1-yl]-3-{[4-(2-ethoxyethoxy)phenyl]amino}pyrazine-2-carboxamide (D-299)

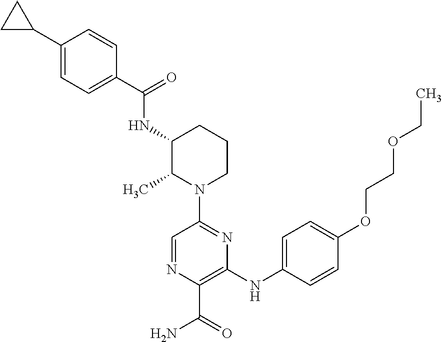 [SMILES: CCOCCOc1ccc(Nc2nc(N3CCC[C@@H](NC(=O)c4ccc(C5CC5)cc4)[C@H]3C)cnc2C(N)=O)cc1]

In a similar manner as described in Example D-269, 5-[(2R,3R)-3-(4-cyclopropylbenzamido)-2-methylpiperidin-1-yl]-3-{[4-(2-ethoxyethoxy)phenyl]amino}pyrazine-2-carboxamide (D-299) was prepared using 4-(2-ethoxyethoxy)aniline. MS found for C31H38N6O4 as (M+H)⁺ 559.2.

¹H NMR (500 MHz, DMSO) δ 11.03 (s, 1H), 8.32 (d, J=7.67 Hz, 1H), 7.83 (d, J=8.55 Hz, 2H), 7.72 (s, 1H), 7.61 (s, 1H), 7.50 (d, J=8.99 Hz, 2H), 7.29 (s, 1H), 7.17 (d, J=8.33 Hz, 2H), 6.82 (d, J=8.77 Hz, 2H), 5.28-4.91 (m, 1H), 4.27-4.00 (m, 2H), 3.99-3.78 (m, 2H), 3.62 (t, J=4.60 Hz, 2H), 3.47 (q, J=7.02 Hz, 2H), 3.05 (t, J=12.28 Hz, 1H), 2.10-1.77 (m, 3H), 1.74-1.52 (m, 2H), 1.19-0.98 (m, 8H), 0.82-0.70 (m, 2H).

### Example D-300: Synthesis of 5-[(2R,3R)-3-(4-cyclopropylbenzamido)-2-methylpiperidin-1-yl]-3-{[(1-methyl-1,2,3,6-tetrahydropyridin-4-yl)methyl]amino}pyrazine-2-carboxamide (D-300)

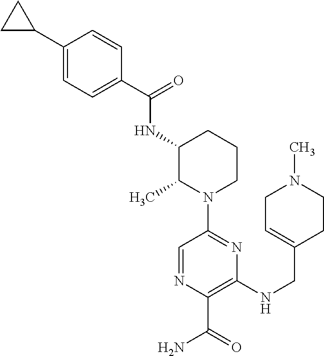 [SMILES: C[C@@H]1[C@H](NC(=O)c2ccc(C3CC3)cc2)CCCN1c1cnc(C(N)=O)c(NCC2=CCN(C)CC2)n1]

In a similar manner as described in Example D-269, 5-[(2R,3R)-3-(4-cyclopropylbenzamido)-2-methylpiperidin-1-yl]-3-{[(1-methyl-1,2,3,6-tetrahydropyridin-4-yl)methyl]amino}pyrazine-2-carboxamide (D-300) was prepared using (1-methyl-1,2,3,6-tetrahydropyridin-4-yl)methanamine. MS found for C28H37N7O2 as (M+H)⁺ 504.5.

¹H NMR (500 MHz, DMSO) δ 8.74 (t, J=5.87 Hz, 1H), 8.25 (d, J=7.43 Hz, 1H), 7.77 (d, J=8.61 Hz, 2H), 7.51 (br. s., 1H), 7.42 (s, 1H), 7.16 (d, J=8.22 Hz, 2H), 7.05 (br. s., 1H), 5.50 (br. s., 1H), 5.07-4.83 (m, 1H), 4.21 (br. s., 1H), 4.04-3.88 (m, 3H), 2.96 (t, J=11.93 Hz, 1H), 2.84 (br. s., 2H), 2.46 (br. s., 2H), 2.22 (s, 3H), 2.10-1.74 (m, 5H), 1.71-1.45 (m, 2H), 1.07 (d, J=7.04 Hz, 3H), 1.04-0.97 (m, 2H), 0.77-0.66 (m, 2H).

### Example D-301: Synthesis of 5-[(2R,3R)-3-(4-cyclopropylbenzamido)-2-methylpiperidin-1-yl]-3-{[4-(1-methylpiperidin-4-yl)phenyl]amino}pyrazine-2-carboxamide (D-301)

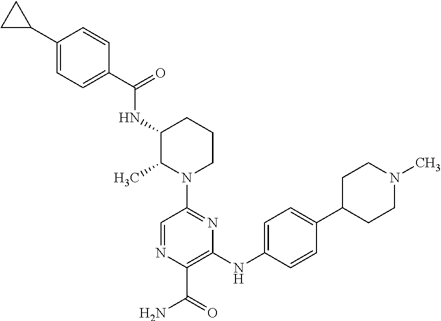 [SMILES: C[C@@H]1[C@H](NC(=O)c2ccc(C3CC3)cc2)CCCN1c1cnc(C(N)=O)c(Nc2ccc(C3CCN(C)CC3)cc2)n1]

In a similar manner as described in Example D-269, 5-[(2R,3R)-3-(4-cyclopropylbenzamido)-2-methylpiperidin-1-yl]-3-{[4-(1-methylpiperidin-4-yl)phenyl]amino}pyrazine-2-carboxamide (D-301) was prepared using 4-(4-aminophenyl)-1-methylpiperidine. MS found for C33H41N7O2 as (M+H)⁺ 568.5.

¹H NMR (500 MHz, DMSO) δ 11.16 (s, 1H), 8.33 (d, J=7.43 Hz, 1H), 7.85 (d, J=8.22 Hz, 2H), 7.75 (br. s., 1H), 7.64 (s, 1H), 7.52 (d, J=8.61 Hz, 2H), 7.32 (br. s., 1H), 7.19 (d, J=8.22 Hz, 2H), 7.10 (d, J=8.22 Hz, 2H), 5.29-4.92 (m, 1H), 4.28-3.93 (m, 2H), 3.07 (t, J=12.13 Hz, 1H), 2.82 (dd, J=6.06, 2.93 Hz, 2H), 2.37-2.26 (m, 1H), 2.20 (s, 3H), 2.06-1.80 (m, 5H), 1.73-1.44 (m, 6H), 1.11-0.95 (m, 5H), 0.76 (s, 1H), 0.81-0.67 (m, 2H).

### Example D-302: Synthesis of 5-[(2R,3R)-3-[2-fluoro-4-(2-hydroxypropan-2-yl)benzamido]-2-methylpiperidin-1-yl]-3-[(1-methyl-1H-pyrazol-4-yl)amino]pyrazine-2-carboxamide (D-302)

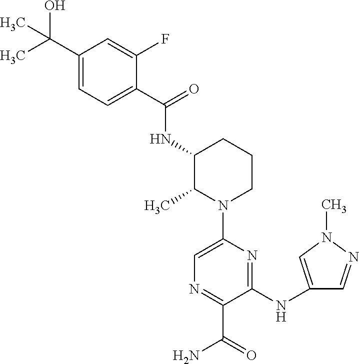 [SMILES: C[C@@H]1[C@H](NC(=O)c2ccc(C(C)(C)O)cc2F)CCCN1c1cnc(C(N)=O)c(Nc2cnn(C)c2)n1]

In a similar manner as described in Example D-216, 5-[(2R,3R)-3-[2-fluoro-4-(2-hydroxypropan-2-yl)benzamido]-2-methylpiperidin-1-yl]-3-[(1-methyl-1H-pyrazol-4-yl)amino]pyrazine-2-carboxamide (D-302) was prepared using 2-fluoro-4-(2-hydroxypropan-2-yl)benzoic acid and 1-methyl-1H-pyrazol-4-amine. MS found for C25H31FN8O3 as (M+H)⁺ 511.4.

¹H NMR (500 MHz, DMSO) δ 10.88 (s, 1H), 8.48-8.39 (m, 1H), 8.03 (s, 1H), 7.69 (br. s., 1H), 7.57 (s, 1H), 7.54-7.48 (m, 1H), 7.47 (s, 1H), 7.39-7.31 (m, 2H), 7.28 (br. s., 1H), 5.44-5.26 (m, 1H), 5.24 (s, 1H), 4.19-4.05 (m, 1H), 4.04-3.93 (m, 1H), 3.76 (s, 3H), 3.13-3.03 (m, 1H), 1.87-1.76 (m, 2H), 1.73-1.53 (m, 2H), 1.43 (s, 6H), 1.12 (d, J=7.04 Hz, 3H).

### Example D-303: Synthesis of 5-[(2R,3R)-3-(4-cyclopropylbenzamido)-2-methylpiperidin-1-yl]-3-[(3,4-dihydro-1H-2-benzopyran-6-yl)amino]pyrazine-2-carboxamide (D-303)

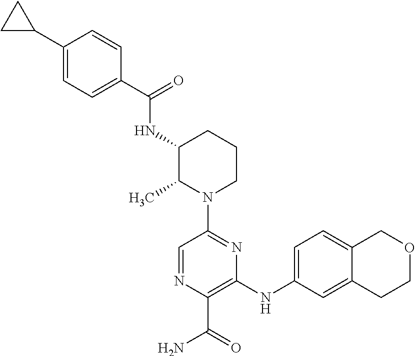 [SMILES: C[C@@H]1[C@H](NC(=O)c2ccc(C3CC3)cc2)CCCN1c1cnc(C(N)=O)c(Nc2ccc3c(c2)CCOC3)n1]

In a similar manner as described in Example D-269, 5-[(2R,3R)-3-(4-cyclopropylbenzamido)-2-methylpiperidin-1-yl]-3-[(3,4-dihydro-1H-2-benzopyran-6-yl)amino]pyrazine-2-carboxamide (D-303) was prepared using 6-aminoisochroman. MS found for C30H34N6O3 as (M+H)⁺ 527.4.

¹H NMR (500 MHz, DMSO) δ 11.29 (s, 1H), 8.37 (d, J=7.45 Hz, 1H), 7.79 (d, J=8.33 Hz, 4H), 7.67 (s, 1H), 7.36 (d, J=2.19 Hz, 1H), 7.18 (d, J=8.33 Hz, 2H), 7.06 (d, J=6.58 Hz, 1H), 6.90 (d, J=8.33 Hz, 1H), 5.14-4.90 (m, 1H), 4.55 (s, 2H), 4.25-3.96 (m, 2H), 3.50 (br. s., 2H), 3.07 (t, J=12.39 Hz, 1H), 2.53-2.39 (m, 2H), 2.07-1.81 (m, 3H), 1.75-1.53 (m, 2H), 1.12 (d, J=7.02 Hz, 3H), 1.06-0.96 (m, 2H), 0.78-0.69 (m, 2H).

### Example D-304: Synthesis of 5-[(2R,3R)-3-{[ethyl(methyl)carbamoyl]amino}-2-methylpiperidin-1-yl]-3-[(1-methyl-1H-pyrazol-4-yl)amino]pyrazine-2-carboxamide (D-304)

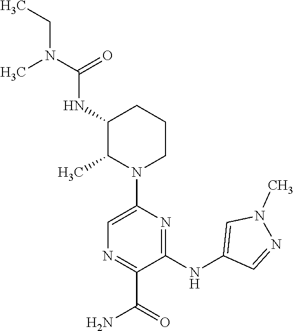 [SMILES: CCN(C)C(=O)N[C@@H]1CCCN(c2cnc(C(N)=O)c(Nc3cnn(C)c3)n2)[C@@H]1C]

In a similar manner as described in Example D-286, 5-[(2R,3R)-3-{[ethyl(methyl)carbamoyl]amino}-2-methylpiperidin-1-yl]-3-[(1-methyl-1H-pyrazol-4-yl)amino]pyrazine-2-carboxamide (D-304) was prepared using N-ethyl-N-methylcarbamoyl chloride. MS found for C19H29N9O2 as (M+H)⁺ 416.4.

¹H NMR (500 MHz, DMSO) δ 10.85 (s, 1H), 8.02 (s, 1H), 7.66 (br. s., 1H), 7.52 (s, 1H), 7.47 (s, 1H), 7.25 (d, J=1.75 Hz, 1H), 6.04 (d, J=6.58 Hz, 1H), 5.31-4.99 (m, 1H), 4.16-3.98 (m, 1H), 3.83 (s, 3H), 3.75-3.64 (m, 1H), 3.42-3.32 (m, 1H), 3.26-3.18 (m, 1H), 3.08-2.96 (m, 1H), 2.82 (s, 3H), 1.90-1.72 (m, 2H), 1.67-1.45 (m, 2H), 1.08-0.97 (m, 6H).

### Example D-305: Synthesis of 5-[(2R,3R)-3-(6-cyclopropylpyridine-3-amido)-2-methylpiperidin-1-yl]-3-{[4-(4,4-difluoropiperidin-1-yl)phenyl]amino}pyrazine-2-carboxamide (D-305)

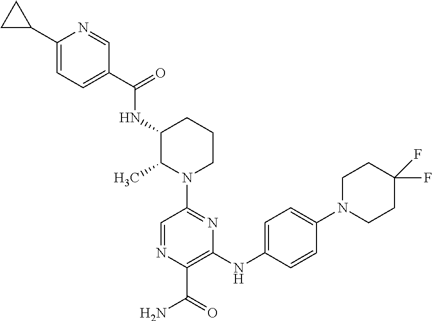 [SMILES: C[C@@H]1[C@H](NC(=O)c2ccc(C3CC3)nc2)CCCN1c1cnc(C(N)=O)c(Nc2ccc(N3CCC(F)(F)CC3)cc2)n1]

In a similar manner as described in Example D-216, 5-[(2R,3R)-3-(6-cyclopropylpyridine-3-amido)-2-methylpiperidin-1-yl]-3-{[4-(4,4-difluoropiperidin-1-yl)phenyl]amino}pyrazine-2-carboxamide (D-305) was prepared using 6-cyclopropylnicotinic acid and 4-(4,4-difluoropiperidin-1-yl)aniline. MS found for C31H36F2N8O22 as (M+H)⁺ 591.4.

¹H NMR (500 MHz, DMSO) δ 10.96 (s, 1H), 8.97-8.83 (m, 1H), 8.49 (d, J=7.45 Hz, 1H), 8.10 (dd, J=8.11, 2.19 Hz, 1H), 7.70 (br. s., 1H), 7.59 (s, 1H), 7.50-7.37 (m, 3H), 7.27 (br. s., 1H), 6.85 (d, J=8.77 Hz, 2H), 5.26-4.93 (m, 1H), 4.23-3.94 (m, 2H), 3.19-2.97 (m, 5H), 2.24-2.11 (m, 1H), 2.04-1.49 (m, 8H), 1.06 (d, J=6.80 Hz, 3H), 1.03-0.93 (m, 4H).

### Example D-306: Synthesis of 3-{[4-(4,4-difluoropiperidin-1-yl)phenyl]amino}-5-[(2R,3R)-3-[(dimethylcarbamoyl)amino]-2-methylpiperidin-1-yl]pyrazine-2-carboxamide (D-306)

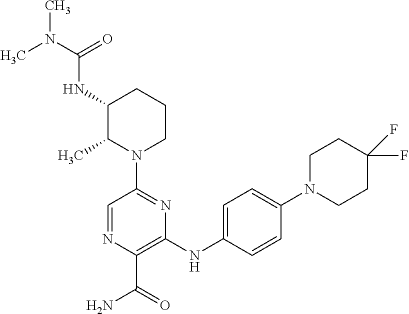 [SMILES: C[C@@H]1[C@H](NC(=O)N(C)C)CCCN1c1cnc(C(N)=O)c(Nc2ccc(N3CCC(F)(F)CC3)cc2)n1]

In a similar manner as described in Example D-286, 3-{[4-(4,4-difluoropiperidin-1-yl)phenyl]amino}-5-[(2R,3R)-3-[(dimethylcarbamoyl)amino]-2-methylpiperidin-1-yl]pyrazine-2-carboxamide (D-306) was prepared using dimethylcarbamyl chloride. MS found for C25H34F2N8O2 as (M+H)⁺ 517.4.

¹H NMR (500 MHz, DMSO) δ 11.07 (s, 1H), 7.70 (br. s., 1H), 7.56 (s, 1H), 7.49 (d, J=8.99 Hz, 2H), 7.27 (d, J=1.97 Hz, 1H), 6.96 (d, J=8.99 Hz, 2H), 6.08 (d, J=7.02 Hz, 1H), 5.07-4.77 (m, 1H), 4.26-4.04 (m, 1H), 3.76-3.64 (m, 1H), 3.27-3.20 (m, 4H), 2.98 (t, J=12.28 Hz, 1H), 2.84 (s, 6H), 2.12-1.98 (m, 4H), 1.88-1.69 (m, 2H), 1.65-1.45 (m, 2H), 1.04 (d, J=6.80 Hz, 3H).

### Example D-307: Synthesis of 5-[(2R,3R)-3-(4-cyclopropylbenzamido)-2-methylpiperidin-1-yl]-3-{[1-(4,4-difluorocyclohexyl)-1H-pyrazol-4-yl]amino}pyrazine-2-carboxamide (D-307)

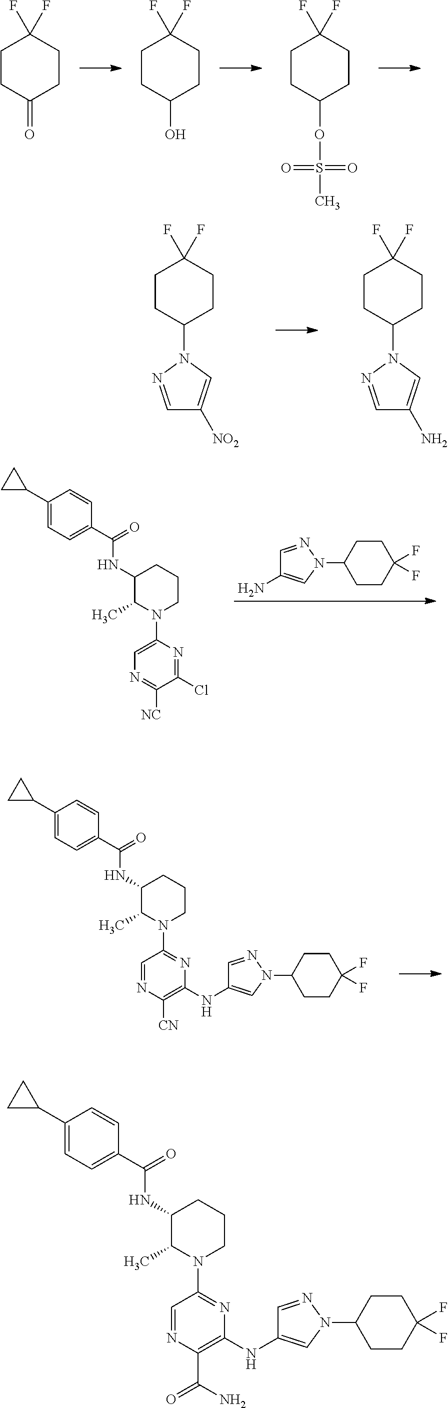

4,4-Difluorocyclohexanone (1 g, 7.46 mmol) was dissolved in EtOH (30 mL). The mixture was cooled to 0° C. and NaBH₄ (560 mg, 14.9 mmol) was added. The mixture was stirred and allowed to warm to room temperature over a period of 3 h. The solvent was removed in vacuo and the residue partitioned between DCM and water. After extraction with DCM, the combined organic extracts were concentrated in vacuo to give 4,4-difluorocyclohexan-1-ol (840 mg, 83% yield) as a clear oil, which was used in the next step without further purification. 4,4-Difluorocyclohexan-1-ol (840 mg, 6.17 mmol) was dissolved in 30 mL of dry DCM, then TEA (1.1 mL, 8.02 mmol) was added. At 0° C., MeSO₂Cl (0.62 mL, 8.02 mmol) was added dropwise and the mixture was stirred at room temperature overnight. The mixture was treated with saturated NaHCO₃ and extracted with DCM. The extracts were dried over Na₂SO₄, filtered and evaporated to give 4,4-difluorocyclohexyl methanesulfonate (1.413 g, quantitative yield) as a yellow solid.

NaH (368 mg, 9.2 mmol) was dissolved under N₂ in DMF (10 mL), the suspension was cooled to 0° C. and a solution of 4-nitro-1H-pyrazole (694 mg, 6.13 mmol) in 10 mL of DMF was added. The solution was stirred at 0° C. for 0.5 h, then a solution of 4,4-difluorocyclohexyl methanesulfonate (1.313 g, 6.13 mmol) in 10 mL of DMF was added. The mixture was heated to room temperature, then at 150° C. for 2 h. Water and ethyl acetate were added and the mixture was extracted with ethyl acetate. The organic phase was dried over Na₂SO₄, filtered and evaporated to give a crude which was purified by silica flash chromatography with 10% to 30% ethyl acetate in cyclohexane to afford 1-(4,4-difluorocyclohexyl)-4-nitro-1H-pyrazole (818.2 mg, 54% yield) as a white solid. MS found for C9H11F2N3O2 as (M+H)⁺ 232.1.

1-(4,4-difluorocyclohexyl)-4-nitro-1H-pyrazole (818.2 mg, 3.54 mmol) was dissolved in 80 mL of EtOH, then Pd/C (170 mg) was added and the mixture was stirred at room temperature under H₂ atmosphere (D-ambient pressure) for 6 h. The catalyst was filtered off then the solvent was evaporated to give 1-(4,4-difluorocyclohexyl)-1H-pyrazol-4-amine (601 mg, 84% yield) as a purple solid, which was used in the next step without further purification. MS found for C9H13F2N3 as (M+H)⁺ 202.1.

In a similar manner as described in Example D-269, 5-[(2R,3R)-3-(4-cyclopropylbenzamido)-2-methylpiperidin-1-yl]-3-{[1-(4,4-difluorocyclohexyl)-1H-pyrazol-4-yl]amino}pyrazine-2-carboxamide (D-306) was prepared using 1-(4,4-difluorocyclohexyl)-1H-pyrazol-4-amine. MS found for C30H36F2N8O2 as (M+H)⁺ 579.4.

¹H NMR (500 MHz, DMSO) δ 10.85 (s, 1H), 8.40 (d, J=7.02 Hz, 1H), 8.04 (s, 1H), 7.82 (d, J=8.33 Hz, 2H), 7.68 (br. s., 1H), 7.59 (s, 1H), 7.48 (s, 1H), 7.28 (br. s., 1H), 7.18 (d, J=8.33 Hz, 2H), 5.40-5.03 (m, 1H), 4.31-3.92 (m, 3H), 3.17-3.04 (m, 1H), 2.08-1.39 (m, 13H), 1.14 (d, J=7.02 Hz, 3H), 1.05-0.95 (m, 2H), 0.77-0.68 (m, 2H).

### Example D-308: Synthesis of 3-[(2R,3R)-3-[2-fluoro-4-(2-hydroxypropan-2-yl)benzamido]-2-methylpiperidin-1-yl]-5-[(1-methyl-1H-pyrazol-4-yl)amino]-1,2,4-triazine-6-carboxamide (D-308)

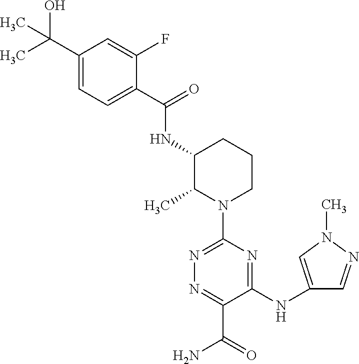 [SMILES: C[C@@H]1[C@H](NC(=O)c2ccc(C(C)(C)O)cc2F)CCCN1c1nnc(C(N)=O)c(Nc2cnn(C)c2)n1]

In a similar manner as described in Example D-298, 3-[(2R,3R)-3-[2-fluoro-4-(2-hydroxypropan-2-yl)benzamido]-2-methylpiperidin-1-yl]-5-[(1-methyl-1H-pyrazol-4-yl)amino]-1,2,4-triazine-6-carboxamide (D-308) was prepared using 2-fluoro-4-(2-hydroxypropan-2-yl)benzoic acid. MS found for C24H30FN9O3 as (M+H)⁺ 512.1.

¹H NMR (500 MHz, DMSO) δ 11.01 (br. s., 1H), 8.56-7.77 (m, 3H), 7.72-7.57 (m, 2H), 7.56-7.46 (m, 1H), 7.39-7.26 (m, 2H), 5.81-5.37 (m, 1H), 5.24 (s, 1H), 5.03-4.24 (m, 1H), 4.16-3.89 (m, 1H), 3.83 (s, 3H), 3.18-2.89 (m, 1H), 1.94-1.49 (m, 4H), 1.43 (s, 6H), 1.14 (d, J=6.80 Hz, 3H).

### Example D-309: Synthesis of 5-[(2R,3R)-3-(6-cyclopropylpyridine-3-amido)-2-methylpiperidin-1-yl]-3-{[4-(oxan-4-yloxy)phenyl]amino}pyrazine-2-carboxamide (D-309)

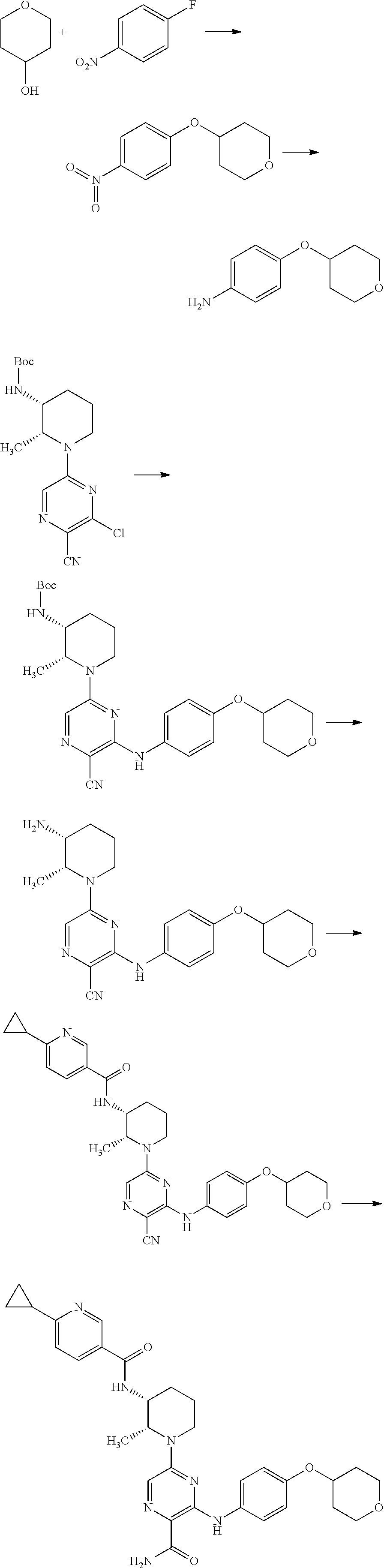

Tetrahydro-4-pyranol (500 mg, 4.9 mmol) was dissolved in 20 mL of THF, then at 0° C., potassium t-butoxyde (1.2 g, 10.8 mmol) was added. The mixture was stirred at 0° C. 20 minutes, then 1-fluoro-4-nitrobenzene (0.6 mL, 5.4 mmol) was added and the mixture was stirred at 0° C. 30 minutes, then at room temperature 4 h. The solvent was evaporated, then water and DCM were added and the mixture was extracted with DCM. The organic phase was dried over Na₂SO₄, filtered and evaporated to give a crude which was purified by silica flash chromatography with 20% to 50% ethyl acetate in cyclohexane, to afford 4-(4-nitrophenoxy)oxane (914.2 mg, 84% yield) as orange solid. MS found for C11H13N04 as (M+H)⁺ 224.1.

4-(4-Nitrophenoxy)oxane (914.2 mg, 4.1 mmol) was dissolved in 80 mL of EtOH, then Pd/C (200 mg) was added and the mixture was stirred at room temperature under H₂ at ambient pressure overnight. The catalyst was filtered off, then the solvent was evaporated to give 4-(oxan-4-yloxy)aniline (710 mg, 90% yield) as a brown solid. MS found for C11H15NO2 as (M+H)⁺ 194.1.

Tert-butyl N-[(2R,3R)-1-(6-chloro-5-cyanopyrazin-2-yl)-2-methylpiperidin-3-yl]carbamate (120 mg, 0.34 mmol) and 4-(oxan-4-yloxy)aniline (79 mg, 0.41 mmol) were dissolved in dioxane (8 mL), (+/−) BINAP (45 mg, 0.068 mmol), Pd(OAc)₂ (15 mg, 0.068 mmol) and Cs₂CO₃ (533 mg, 1.64 mmol) were added. The mixture was stirred at 100° C. 2 h. Ethyl acetate was added and the mixture was filtered on a celite pad, the filtrate was evaporated to give a crude which was purified by silica flash chromatography with 20% to 60% ethyl acetate in cyclohexane to afford tert-butyl N-[(2R,3R)-1-(5-cyano-6-{[4-(oxan-4-yloxy)phenyl]amino}pyrazin-2-yl)-2-methylpiperidin-3-yl]carbamate (139.8 mg, 81% yield) as a yellow solid. MS found for C27H36N6O4 as (M+H)⁺ 509.4.

Tert-butyl N-[(2R,3R)-1-(5-cyano-6-{[4-(oxan-4-yloxy)phenyl]amino}pyrazin-2-yl)-2-methylpiperidin-3-yl]carbamate (139.8 mg, 0.27 mmol) was dissolved in 4 mL of DCM, then TFA (0.4 ml) was added and the mixture was stirred at room temperature 2 h. The solvent was evaporated then the crude was purified by SCX to afford 5-[(2R,3R)-3-amino-2-methylpiperidin-1-yl]-3-{[4-(oxan-4-yloxy)phenyl]amino}pyrazine-2-carbonitrile (102.8 mg, 92% yield) as a yellow solid. MS found for C22H28N6O2 as (M+H)⁺ 409.0.

5-[(2R,3R)-3-amino-2-methylpiperidin-1-yl]-3-{[4-(oxan-4-yloxy)phenyl]amino}pyrazine-2-carbonitrile (102.8 mg, 0.25 mmol) was dissolved in DMF (4 mL), 6-cyclopropylnicotinic acid (62 mg, 0.38 mmol), DIPEA (0.2 mL, 1.26 mmol) and PyBOP (196 mg, 0.38 mmol) were added. The mixture was stirred at room temperature 1 h. Water and DCM were added and the mixture was extracted with DCM. The organic phase was dried over Na₂SO₄, filtered and evaporated to give a crude which was purified by silica flash chromatography with 50% to 100% ethyl acetate in cyclohexane to afford N-[(2R,3R)-1-(5-cyano-6-{[4-(oxan-4-yloxy)phenyl]amino}pyrazin-2-yl)-2-methylpiperidin-3-yl]-6-cyclopropylpyridine-3-carboxamide (120.7 mg, 87% yield) as a yellow solid. MS found for C31H35N7O3 as (M+H)⁺ 554.0.

N-[(2R,3R)-1-(5-cyano-6-{[4-(oxan-4-yloxy)phenyl]amino}pyrazin-2-yl)-2-methylpiperidin-3-yl]-6-cyclopropylpyridine-3-carboxamide (120.7 mg, 0.2 mmol) was dissolved in MeOH (4 mL), TEA (1 mL), DMSO (0.4 mL), NaOH (21 mg, 0.52 mmol) and H₂O₂ 0% (0.2 mL) were added. The mixture was stirred at room temperature 2 h, then water and DCM were added and the mixture was extracted with DCM. The organic phase was dried over Na₂SO₄, filtered and evaporated to give a crude which was purified by SCX, to afford 5-[(2R,3R)-3-(6-cyclopropylpyridine-3-amido)-2-methylpiperidin-1-yl]-3-{[4-(oxan-4-yloxy)phenyl]amino}pyrazine-2-carboxamide (86.6 mg, 69% yield) as a yellow solid. MS found for C31H37N7O4 as (M+H)⁺ 572.1.

¹H NMR (500 MHz, DMSO) δ 11.06 (s, 1H), 8.89 (d, J=1.97 Hz, 1H), 8.48 (d, J=7.24 Hz, 1H), 8.11 (dd, J=8.11, 2.19 Hz, 1H), 7.73 (br. s., 1H), 7.62 (s, 1H), 7.51 (d, J=8.99 Hz, 2H), 7.42 (d, J=8.33 Hz, 1H), 7.30 (br. s., 1H), 6.85 (d, J=8.77 Hz, 2H), 5.32-4.91 (m, 1H), 4.32 (br. s., 1H), 4.24-3.98 (m, 2H), 3.86-3.71 (m, 2H), 3.46-3.33 (m, 2H), 3.06 (t, J=12.39 Hz, 1H), 2.24-2.13 (m, 1H), 1.99-1.79 (m, 4H), 1.73-1.44 (m, 4H), 1.09 (d, J=6.80 Hz, 3H), 1.04-0.95 (m, 4H).

### Example D-310: Synthesis of 5-[(2R,3R)-3-(4-cyclopropylbenzamido)-2-methylpiperidin-1-yl]-3-{[3-(oxan-4-yl)-1,2-thiazol-5-yl]amino}pyrazine-2-carboxamide (D-310)

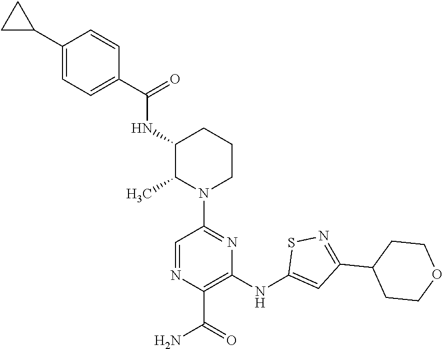 [SMILES: C[C@@H]1[C@H](NC(=O)c2ccc(C3CC3)cc2)CCCN1c1cnc(C(N)=O)c(Nc2cc(C3CCOCC3)ns2)n1]

In a similar manner as described in Example D-269, 5-[(2R,3R)-3-(4-cyclopropylbenzamido)-2-methylpiperidin-1-yl]-3-{[3-(oxan-4-yl)-1,2-thiazol-5-yl]amino}pyrazine-2-carboxamide (D-310) was prepared using 3-(oxan-4-yl)-1,2-thiazol-5-amine. MS found for C29H35N7O3S as (M+H)⁺ 562.2.

¹H NMR (500 MHz, DMSO) δ 12.31 (s, 1H), 8.35 (d, J=6.85 Hz, 1H), 7.92 (br. s., 1H), 7.85-7.69 (m, 3H), 7.56 (br. s., 1H), 7.17 (d, J=7.83 Hz, 2H), 6.96 (s, 1H), 5.47-4.16 (m, 2H), 4.13-3.99 (m, 1H), 3.89 (d, J=10.27 Hz, 2H), 3.40 (t, J=11.49 Hz, 2H), 3.17 (t, J=12.72 Hz, 1H), 2.86 (t, J=11.74 Hz, 1H), 2.11-1.55 (m, 9H), 1.22 (d, J=6.85 Hz, 3H), 1.07-0.93 (m, 2H), 0.74 (d, J=5.38 Hz, 2H).

### Example D-311: Synthesis of 3-{[3-(1-cyclopentylpiperidin-4-yl)-1,2-thiazol-5-yl]amino}-5-[(2R,3R)-3-(4-cyclopropylbenzamido)-2-methylpiperidin-1-yl]pyrazine-2-carboxamide (D-311)

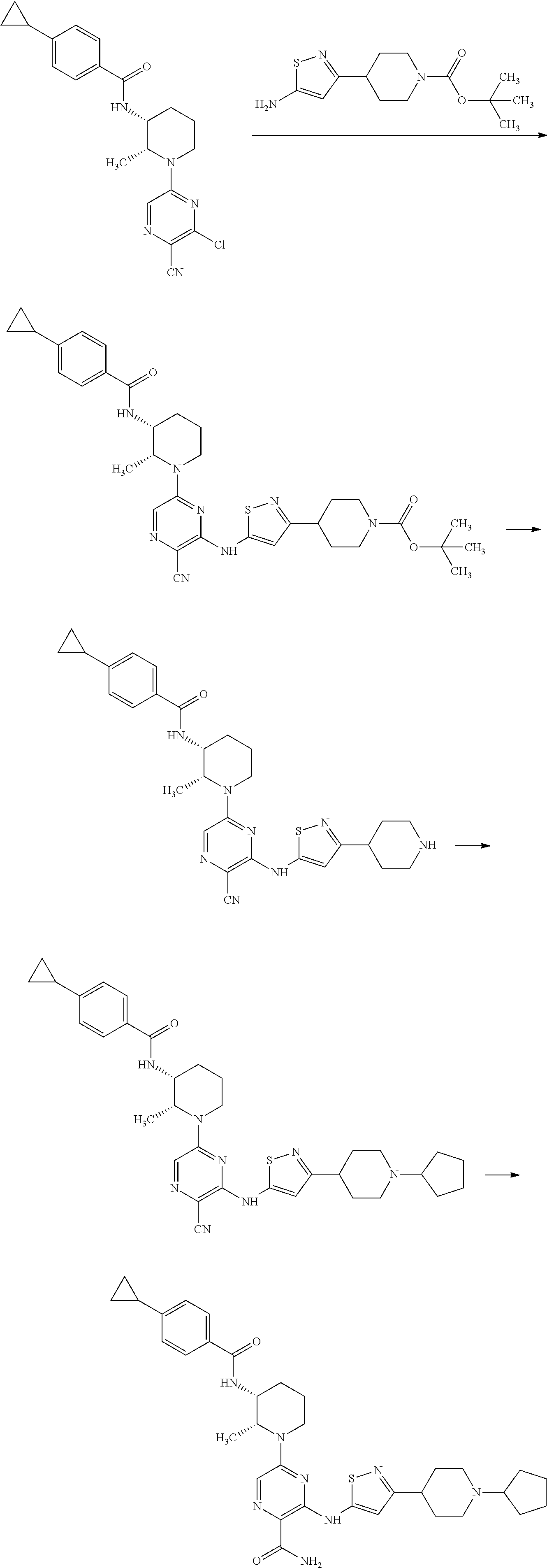 [SMILES: C[C@@H]1[C@H](NC(=O)c2ccc(C3CC3)cc2)CCCN1c1cnc(C#N)c(Cl)n1 ; CC(C)(C)OC(=O)N1CCC(c2cc(N)sn2)CC1 ; C[C@@H]1[C@H](NC(=O)c2ccc(C3CC3)cc2)CCCN1c1cnc(C#N)c(Nc2cc(C3CCN(C(=O)OC(C)(C)C)CC3)ns2)n1 ; C[C@@H]1[C@H](NC(=O)c2ccc(C3CC3)cc2)CCCN1c1cnc(C#N)c(Nc2cc(C3CCNCC3)ns2)n1 ; C[C@@H]1[C@H](NC(=O)c2ccc(C3CC3)cc2)CCCN1c1cnc(C#N)c(Nc2cc(C3CCN(C4CCCC4)CC3)ns2)n1 ; C[C@@H]1[C@H](NC(=O)c2ccc(C3CC3)cc2)CCCN1c1cnc(C(N)=O)c(Nc2cc(C3CCN(C4CCCC4)CC3)ns2)n1]

N-[(2R,3R)-1-(6-chloro-5-cyanopyrazin-2-yl)-2-methylpiperidin-3-yl]-4-cyclopropylbenzamide (300 mg, 0.75 mmol) and tert-butyl 4-(5-amino-1,2-thiazol-3-yl)piperidine-1-carboxylate (258 mg, 0.91 mmol) were dissolved in 10 mL of dioxane not dry, (+/−) BINAP (100 mg, 0.15 mmol), Pd(OAc)₂ (34 mg, 0.15 mmol) and Cs₂CO₃ (1.185 g, 3.63 mmol) were added. The mixture was stirred at 100° C. 2 h, then ethyl acetate was added and the mixture was filtered on a celite pad, the filtrate was evaporated to afford a crude which was purified by silica flash chromatography with 30% to 80% ethyl acetate in cyclohexane to obtain tert-butyl 4-[5-({3-cyano-6-[(2R,3R)-3-(4-cyclopropylbenzamido)-2-methylpiperidin-1-yl]pyrazin-2-yl}amino)-1,2-thiazol-3-yl]piperidine-1-carboxylate (262.3 mg, 54% yield) as a pale yellow solid. MS found for C34H42N8O3S as (M+H)⁺ 643.5.

Tert-butyl 4-[5-({3-cyano-6-[(2R,3R)-3-(4-cyclopropylbenzamido)-2-methylpiperidin-1-yl]pyrazin-2-yl}amino)-1,2-thiazol-3-yl]piperidine-1-carboxylate (262.3 mg, 0.41 mmol) was dissolved in 4 mL of DCM, then TFA (0.6 mL) was added and the mixture was stirred at room temperature 2 h. The solvent was evaporated, the residue was purified by SCX to obtain N-[(2R,3R)-1-(5-cyano-6-{[3-(piperidin-4-yl)-1,2-thiazol-5-yl]amino}pyrazin-2-yl)-2-methylpiperidin-3-yl]-4-cyclopropylbenzamide (211.9 mg, 96% yield) as a yellow solid. MS found for C29H34N8OS as (M+H)⁺ 543.2.

N-[(2R,3R)-1-(5-cyano-6-{[3-(piperidin-4-yl)-1,2-thiazol-5-yl]amino}pyrazin-2-yl)-2-methylpiperidin-3-yl]-4-cyclopropylbenzamide (106 mg, 0.2 mmol) was dissolved in MeOH (4 mL), then cyclopentanone (0.02 mL, 0.2 mmol) was added and the mixture was stirred at room temperature 15 minutes. Then, NaBH₃CN (31 mg, 0.49 mmol) was added and the solution stirred at room temperature overnight. The solvent was evaporated to give a crude which was purified by silica flash chromatography with 0% to 20% MeOH in DCM to obtain N-[(2R,3R)-1-(5-cyano-6-{[3-(1-cyclopentylpiperidin-4-yl)-1,2-thiazol-5-yl]amino}pyrazin-2-yl)-2-methylpiperidin-3-yl]-4-cyclopropylbenzamide (58.9 mg, 49% yield) as a yellow solid. MS found for C34H42N8OS as (M+H)⁺ 611.2.

N-[(2R,3R)-1-(5-cyano-6-{[3-(1-cyclopentylpiperidin-4-yl)-1,2-thiazol-5-yl]amino}pyrazin-2-yl)-2-methylpiperidin-3-yl]-4-cyclopropylbenzamide (58.9 mg, 0.096 mmol) was dissolved in 2 mL of MeOH, TEA (0.5 mL), DMSO (0.2 mL), NaOH (20 mg) and H₂O₂ 30% (0.1 mL) were added. The mixture was stirred at room temperature overnight. Water and DCM were added and the mixture was extracted with DCM. Organic phase was evaporated to give a crude which was purified by SCX and then by silica flash chromatography with 0% to 20% MeOH in DCM to obtain 3-{[3-(1-cyclopentylpiperidin-4-yl)-1,2-thiazol-5-yl]amino}-5-[(2R,3R)-3-(4-cyclopropylbenzamido)-2-methylpiperidin-1-yl]pyrazine-2-carboxamide (36.9 mg, 61% yield) as a pale yellow solid. MS found for C34H44N8O2S as (M+H)⁺ 629.5.

¹H NMR (500 MHz, DMSO) δ 12.30 (s, 1H), 8.34 (d, J=7.45 Hz, 1H), 7.91 (br. s., 1H), 7.85-7.73 (m, 3H), 7.55 (br. s., 1H), 7.17 (d, J=8.33 Hz, 2H), 6.94 (s, 1H), 5.31-4.70 (m, 1H), 4.65-4.17 (m, 1H), 4.12-3.97 (m, 1H), 3.21-2.54 (m, 5H), 2.29-1.28 (m, 19H), 1.22 (d, J=6.80 Hz, 3H), 1.06-0.96 (m, 2H), 0.79-0.68 (m, 2H).

### Example D-312: Synthesis of 5-[(2R,3R)-3-(4-cyclopropylbenzamido)-2-methylpiperidin-1-yl]-3-{[3-(1-methylpiperidin-4-yl)-1,2-thiazol-5-yl]amino}pyrazine-2-carboxamide (D-312)

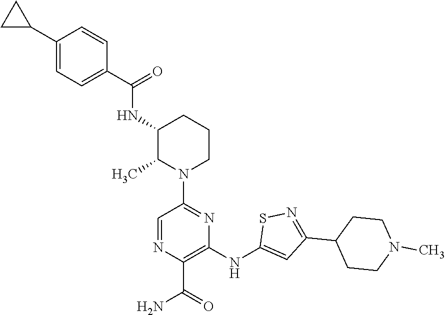 [SMILES: C[C@@H]1[C@H](NC(=O)c2ccc(C3CC3)cc2)CCCN1c1cnc(C(N)=O)c(Nc2cc(C3CCN(C)CC3)ns2)n1]

In a similar manner as described in Example D-311, 5-[(2R,3R)-3-(4-cyclopropylbenzamido)-2-methylpiperidin-1-yl]-3-{[3-(1-methylpiperidin-4-yl)-1,2-thiazol-5-yl]amino}pyrazine-2-carboxamide (D-312) was prepared using formaldehyde. MS found for C30H38N8O2S as (M+H)⁺ 575.4.

¹H NMR (500 MHz, DMSO) δ 12.29 (s, 1H), 8.34 (d, J=7.04 Hz, 1H), 7.91 (br. s., 1H), 7.84-7.75 (m, 3H), 7.56 (br. s., 1H), 7.17 (d, J=8.22 Hz, 2H), 6.93 (s, 1H), 5.04 (br. s., 1H), 4.44 (br. s., 1H), 4.17-3.99 (m, 1H), 3.22-3.10 (m, 1H), 2.83 (d, J=11.54 Hz, 2H), 2.19 (s, 3H), 2.10-1.57 (m, 12H), 1.22 (d, J=6.85 Hz, 3H), 1.07-0.96 (m, 2H), 0.79-0.71 (m, 2H).

### Example D-313: Synthesis of 5-[(2R,3R)-3-(4-cyclopropyl-2-fluorobenzamido)-2-methylpiperidin-1-yl]-3-{[3-fluoro-4-(piperazin-1-yl)phenyl]amino}pyrazine-2-carboxamide (D-313)

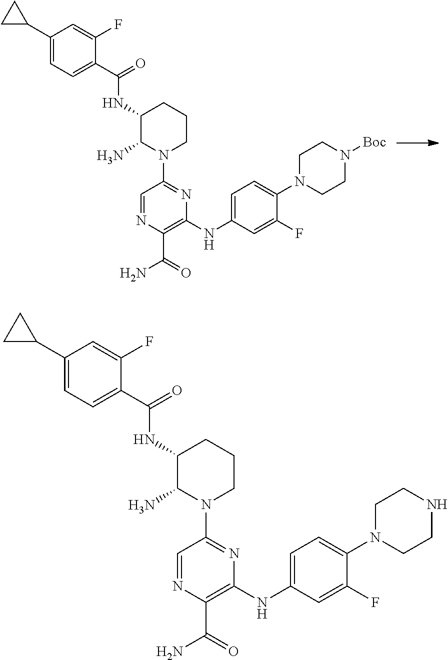 [SMILES: CC(C)(C)OC(=O)N1CCN(c2ccc(Nc3nc(N4CCC[C@@H](NC(=O)c5ccc(C6CC6)cc5F)[C@H]4N)cnc3C(N)=O)cc2F)CC1 ; NC(=O)c1ncc(N2CCC[C@@H](NC(=O)c3ccc(C4CC4)cc3F)[C@H]2N)nc1Nc1ccc(N2CCNCC2)c(F)c1]

In a similar manner as described in Example D-269, 5-[(2R,3R)-3-(4-cyclopropyl-2-fluorobenzamido)-2-methylpiperidin-1-yl]-3-{[3-fluoro-4-(piperazin-1-yl)phenyl]amino}pyrazine-2-carboxamide (D-313) was prepared using 4-(4-Boc-piperazin-1-yl)-3-fluoroaniline. Tert-butyl-4-[4-((3-carbamoyl-6-[(2R,3R)-3-(4-cyclopropyl-2-fluorobenzamido)-2-methylpiperidin-1-yl]pyrazin-2-yl) amino)-2-fluorophenyl]piperazine-1-carboxylate (107 mg) was dissolved in 3 mL of DCM, then TFA (0.5 mL) was added and the mixture was stirred at room temperature 2 h. The solvent was evaporated, then the crude was charged on a SCX cartridge eluting with NH₃ 7 M in MeOH and DCM. The compound was further purified by preparative HPLC to obtain 5-[(2R,3R)-3-(4-cyclopropyl-2-fluorobenzamido)-2-methylpiperidin-1-yl]-3-{[3-fluoro-4-(piperazin-1-yl)phenyl]amino}pyrazine-2-carboxamide (D-313, 22.2 mg, 24% yield) as a yellow solid. MS found for C31H36F2N8O2 as (M+H)⁺ 591.4.

¹H NMR (500 MHz, DMSO) δ 11.19 (s, 1H), 8.29 (d, J=7.43 Hz, 1H), 7.76 (br. s., 1H), 7.66 (s, 1H), 7.53-7.41 (m, 2H), 7.39-7.33 (m, 1H), 7.01 (s, 4H), 5.16-4.90 (m, 1H), 4.21-3.93 (m, 2H), 3.06 (t, J=12.33 Hz, 1H), 2.78 (s, 8H), 2.07-1.96 (m, 1H), 1.84 (br. s., 4H), 1.15 (d, J=6.65 Hz, 3H), 1.09-0.98 (m, 2H), 0.79-0.72 (m, 2H).

### Example D-314: Synthesis of 5-[(2R,3R)-3-{[cyclopropyl(methyl)carbamoyl]amino}-2-methylpiperidin-1-yl]-3-[(1-methyl-1H-pyrazol-4-yl)amino]pyrazine-2-carboxamide (D-314)

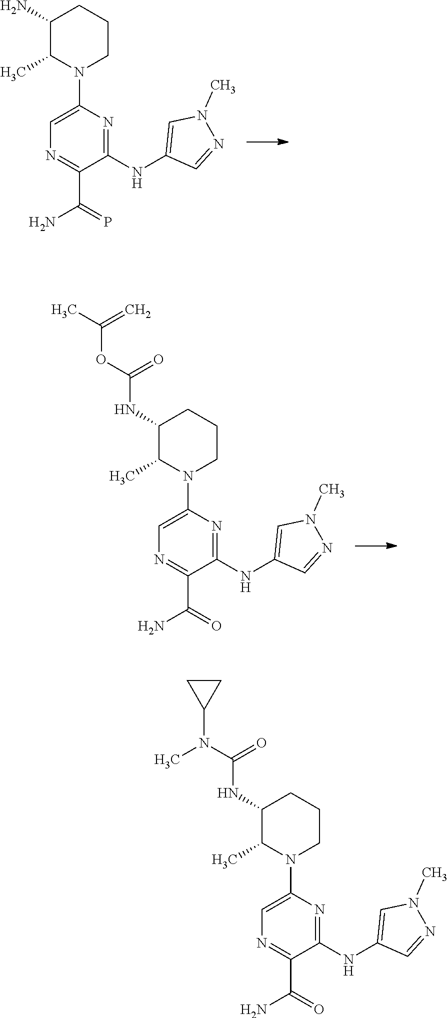

5-[(2R,3R)-3-amino-2-methylpiperidin-1-yl]-3-[(1-methyl-1H-pyrazol-4-yl)amino]pyrazine-2-carboxamide (140 mg, 0.3 mmol) was dissolved in DCM (4 mL), then DIPEA (0.15 mL, 0.84 mmol) was added, followed by isopropenyl chloroformate at 0° C. (0.05 mL, m 0.47 mmol). The reaction was stirred at room temperature 2 h. NaHCO₃ saturated solution and DCM were added and the mixture was extracted with DCM. The organic phase was dried over Na₂SO₄, filtered and evaporated to give prop-1-en-2-yl N-[(2R,3R)-1-{5-carbamoyl-6-[(1-methyl-1H-pyrazol-4-yl)amino]pyrazin-2-yl}-2-methylpiperidin-3-yl]carbamate (197.7 mg) which was used in the next step without further purification. MS found for C19H26N8O3 as (M+H)⁺ 415.1. Prop-1-en-2-yl N-[(2R,3R)-1-{5-carbamoyl-6-[(1-methyl-1H-pyrazol-4-yl)amino]pyrazin-2-yl}-2-methylpiperidin-3-yl]carbamate (197.7 mg, 0.48 mmol) was dissolved in 4 mL of DMF in a microwave tube, then DIPEA (0.2 mL, 0.95 mmol) was added, followed by N-cyclopropylmethylamine (0.04 mL, 0.48 mmol). The microwave tube containing the reaction mixture was heated under microwave at 140° C. for 3 cycles of 20 minutes each, then at 160° C. for 30 minutes. Water and ethyl acetate were added and the mixture was extracted with ethyl acetate. The organic phase was dried over Na₂SO₄, filtered and evaporated to give a crude which was purified by silica flash chromatography with 0% to 10% MeOH in DCM to obtain 5-[(2R,3R)-3-{[cyclopropyl(methyl)carbamoyl]amino}-2-methylpiperidin-1-yl]-3-[(1-methyl-1H-pyrazol-4-yl)amino]pyrazine-2-carboxamide (95.8 mg, 47% yield) as a yellow solid. MS found for C20H29N9O2 as (M+H)⁺ 428.1.

¹H NMR (500 MHz, DMSO) δ 10.85 (s, 1H), 8.02 (s, 1H), 7.67 (br. s., 1H), 7.53 (s, 1H), 7.46 (s, 1H), 7.27 (d, J=1.37 Hz, 1H), 5.87 (d, J=6.59 Hz, 1H), 5.16 (br. s., 1H), 4.17-3.99 (m, 1H), 3.83 (s, 3H), 3.77-3.67 (m, 1H), 3.08-2.98 (m, 1H), 2.80 (s, 3H), 2.58-2.50 (m, 1H), 1.81 (d, J=12.35 Hz, 4H), 1.07 (d, J=6.86 Hz, 3H), 0.91-0.57 (m, 4H).

### Example D-315: Synthesis of 5-[(2R,3R)-3-(4-cyclopropylbenzamido)-2-methylpiperidin-1-yl]-3-[(2,2-difluoro-2H-1,3-benzodioxol-5-yl)amino]pyrazine-2-carboxamide (D-315)

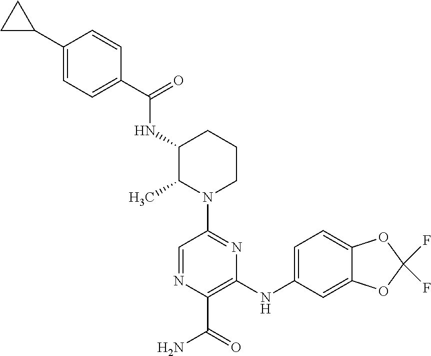 [SMILES: C[C@@H]1[C@H](NC(=O)c2ccc(C3CC3)cc2)CCCN1c1cnc(C(N)=O)c(Nc2ccc3c(c2)OC(F)(F)O3)n1]

In a similar manner as described in Example D-269, 5-[(2R,3R)-3-(4-cyclopropylbenzamido)-2-methylpiperidin-1-yl]-3-[(2,2-difluoro-2H-1,3-benzodioxol-5-yl)amino]pyrazine-2-carboxamide (D-315) was prepared using 2,2-difluoro-5-aminobenzodioxole. MS found for C28H28F2N6O4 as (M+H)⁺ 551.4.

¹H NMR (500 MHz, DMSO) δ 11.48 (s, 1H), 8.31 (d, J=7.24 Hz, 1H), 7.87 (d, J=1.97 Hz, 1H), 7.84-7.76 (m, 3H), 7.73-7.68 (m, 1H), 7.44-7.38 (m, 1H), 7.31-7.21 (m, 2H), 7.18-7.13 (m, 2H), 5.41-4.85 (m, 1H), 4.36-3.88 (m, 2H), 3.09 (t, J=11.95 Hz, 1H), 2.15-1.49 (m, 5H), 1.11 (d, J=7.02 Hz, 3H), 1.05-0.97 (m, 2H), 0.77-0.69 (m, 2H).

### Example D-316: Synthesis of 5-[(2R,3R)-3-(4-cyclopropylbenzamido)-2-methylpiperidin-1-yl]-3-{[4-(morpholin-4-ylmethyl)phenyl]amino}pyrazine-2-carboxamide (D-316)

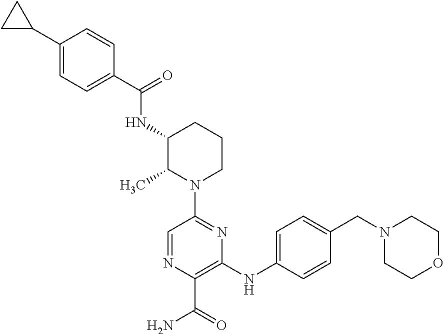 [SMILES: C[C@@H]1[C@H](NC(=O)c2ccc(C3CC3)cc2)CCCN1c1cnc(C(N)=O)c(Nc2ccc(CN3CCOCC3)cc2)n1]

In a similar manner as described in Example D-269, 5-[(2R,3R)-3-(4-cyclopropylbenzamido)-2-methylpiperidin-1-yl]-3-{[4-(morpholin-4-ylmethyl)phenyl]amino}pyrazine-2-carboxamide (D-316) was prepared using 4-(morpholinomethyl)aniline. MS found for C32H39N7O3 as (M+H)⁺ 570.2.

¹H NMR (500 MHz, DMSO) δ 11.29 (s, 1H), 8.31 (d, J=7.45 Hz, 1H), 7.83 (d, J=8.11 Hz, 2H), 7.77 (br. s., 1H), 7.66 (s, 1H), 7.57 (d, J=8.55 Hz, 2H), 7.35 (br. s., 1H), 7.23-7.13 (m, 4H), 5.36-4.87 (m, 1H), 4.34-3.97 (m, 2H), 3.51 (t, J=4.38 Hz, 4H), 3.34 (s, 2H), 3.14-3.00 (m, 1H), 2.29 (br. s., 4H), 2.08-1.52 (m, 5H), 1.09 (d, J=6.80 Hz, 3H), 1.05-0.97 (m, 2H), 0.78-0.71 (m, 2H).

### Example D-317: Synthesis of 5-[(2R,3R)-3-(4-cyclopropylbenzamido)-2-methylpiperidin-1-yl]-3-{[4-(oxan-4-yl)phenyl]amino}pyrazine-2-carboxamide (D-317)

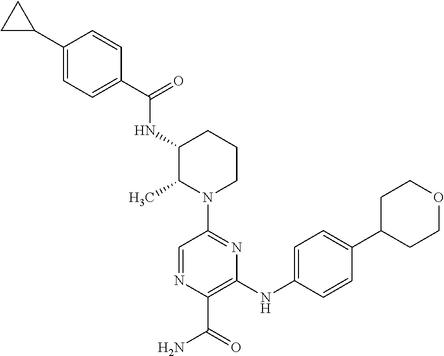 [SMILES: C[C@@H]1[C@H](NC(=O)c2ccc(C3CC3)cc2)CCCN1c1cnc(C(N)=O)c(Nc2ccc(C3CCOCC3)cc2)n1]

In a similar manner as described in Example D-269, 5-[(2R,3R)-3-(4-cyclopropylbenzamido)-2-methylpiperidin-1-yl]-3-{[4-(oxan-4-yl)phenyl]amino}pyrazine-2-carboxamide (D-317) was prepared using 4-(oxan-4-yl)aniline. MS found for C32H38N6O3 as (M+H)⁺ 555.2.

¹H NMR (500 MHz, DMSO) δ 11.18 (s, 1H), 8.33 (d, J=7.43 Hz, 1H), 7.84 (d, J=8.61 Hz, 2H), 7.75 (br. s., 1H), 7.64 (s, 1H), 7.54 (d, J=8.61 Hz, 2H), 7.33 (d, J=1.96 Hz, 1H), 7.18 (d, J=8.61 Hz, 2H), 7.12 (d, J=8.22 Hz, 2H), 5.12 (br. s., 1H), 4.27-3.99 (m, 2H), 3.96-3.83 (m, 2H), 3.46-3.35 (m, 2H), 3.07 (t, J=12.13 Hz, 1H), 2.70-2.55 (m, 1H), 2.07-1.43 (m, 9H), 1.08 (d, J=6.65 Hz, 3H), 1.07-0.98 (m, 2H), 0.81-0.70 (m, 2H).

### Example D-318: Synthesis of 5-[(2R,3R)-3-(4-cyclopropylbenzamido)-2-methylpiperidin-1-yl]-3-{[2-(4-methylpiperazin-1-yl)pyrimidin-5-yl]amino}pyrazine-2-carboxamide (D-318)

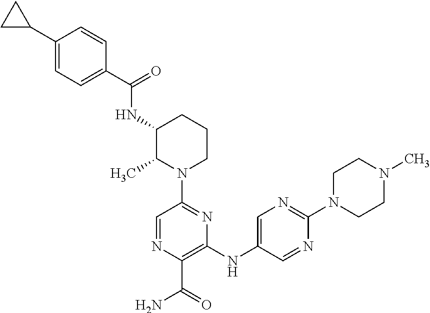 [SMILES: C[C@@H]1[C@H](NC(=O)c2ccc(C3CC3)cc2)CCCN1c1cnc(C(N)=O)c(Nc2cnc(N3CCN(C)CC3)nc2)n1]

In a similar manner as described in Example D-269, 5-[(2R,3R)-3-(4-cyclopropylbenzamido)-2-methylpiperidin-1-yl]-3-{[2-(4-methylpiperazin-1-yl)pyrimidin-5-yl]amino}pyrazine-2-carboxamide (D-318) was prepared using 2-(4-methylpiperazin-1-yl)pyrimidin-5-amine. MS found for C30H38N10O2 as (M+H)⁺ 571.5.

¹H NMR (500 MHz, DMSO) δ 10.70 (s, 1H), 8.55 (s, 2H), 8.28 (d, J=7.67 Hz, 1H), 7.88-7.70 (m, 3H), 7.64 (s, 1H), 7.33 (br. s., 1H), 7.15 (d, J=8.55 Hz, 2H), 4.99-4.73 (m, 1H), 4.28-4.10 (m, 1H), 4.07-3.95 (m, 1H), 3.57 (br. s., 4H), 3.10-2.94 (m, 1H), 2.27 (br. s., 4H), 2.18 (s, 3H), 2.03-1.77 (m, 3H), 1.71-1.48 (m, 2H), 1.10 (d, J=6.80 Hz, 3H), 1.05-0.98 (m, 2H), 0.78-0.70 (m, 2H).

### Example D-319: Synthesis of 5-[(2R,3R)-3-(4-cyclopropylbenzamido)-2-methylpiperidin-1-yl]-3-{[1-(oxan-4-ylmethyl)-1H-pyrazol-4-yl]amino}pyrazine-2-carboxamide (D-319)

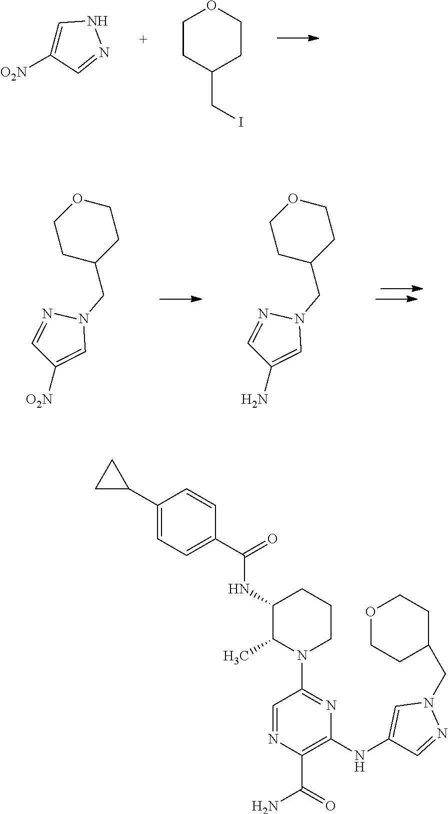 [SMILES: O=[N+]([O-])c1cn[nH]c1 ; ICC1CCOCC1 ; O=[N+]([O-])c1cnn(CC2CCOCC2)c1 ; Nc1cnn(CC2CCOCC2)c1 ; C[C@@H]1[C@H](NC(=O)c2ccc(C3CC3)cc2)CCCN1c1cnc(C(N)=O)c(Nc2cnn(CC3CCOCC3)c2)n1]

4-Nitropyrazole (300 mg, 2.65 mmol) and 4-(iodomethyl)tetrahydro-2H-pyran (600 mg, 2.65 mmol) were dissolved in 10 mL of DMF with 1.7 g (5.3 mmol) of Cs₂CO₃, then the mixture so obtained was stirred at 80° C. 5 h. The mixture was cooled to room temperature, then it was extracted with DCM. The organic phase was dried over Na₂SO₄, filtered and evaporated to give a crude which was purified by silica flash chromatography with 30% to 80% ethyl acetate in cyclohexane to obtain 4-nitro-1-(oxan-4-ylmethyl)-1H-pyrazole (488.4 mg, 87% yield) as a colorless oil. MS found for C9H13N3O3 as (M+H)⁺ 212.1.

4-Nitro-1-(oxan-4-ylmethyl)-1H-pyrazole (488.4 mg, 2.31 mmol) was dissolved in EtOH (20 mL), then Pd/C (100 mg) was added and the mixture was stirred under H₂ at ambient pressure 2 h. The catalyst was filtered off and the solvent was evaporated to give 1-(oxan-4-ylmethyl)-1H-pyrazol-4-amine (393 mg, 94% yield) as a purple oil which was used in the next step without further purification. MS found for C9H15N30 as (M+H)⁺ 182.0.

In a similar manner as described in Example D-269, 5-[(2R,3R)-3-(4-cyclopropylbenzamido)-2-methylpiperidin-1-yl]-3-{[1-(oxan-4-ylmethyl)-1H-pyrazol-4-yl]amino}pyrazine-2-carboxamide (D-319) was prepared using 1-(oxan-4-ylmethyl)-1H-pyrazol-4-amine. MS found for C30H38N8O3 as (M+H)⁺ 559.2.

¹H NMR (500 MHz, DMSO) δ 10.86 (s, 1H), 8.42 (d, J=6.59 Hz, 1H), 8.07 (s, 1H), 7.83 (d, J=8.23 Hz, 2H), 7.68 (br. s., 1H), 7.57 (s, 1H), 7.43 (s, 1H), 7.27 (br. s., 1H), 7.18 (d, J=8.23 Hz, 2H), 5.57-5.15 (m, 1H), 4.22-3.98 (m, 2H), 3.90-3.71 (m, 2H), 3.63 (d, J=9.33 Hz, 2H), 3.18-2.97 (m, 3H), 1.98 (dd, J=4.80, 3.16 Hz, 6H), 1.19-0.67 (m, 11H).

### Example D-320: Synthesis of 5-[(2R,3R)-3-(4-cyclopropylbenzamido)-2-methylpiperidin-1-yl]-3-({4-[4-(dimethylcarbamoyl)piperidin-1-yl]phenyl}amino)pyrazine-2-carboxamide (D-320)

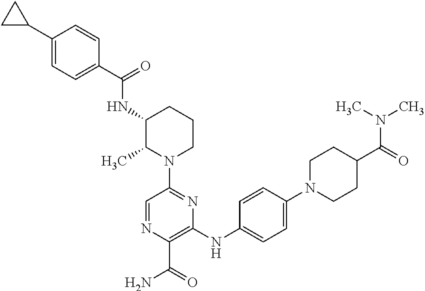 [SMILES: C[C@@H]1[C@H](NC(=O)c2ccc(C3CC3)cc2)CCCN1c1cnc(C(N)=O)c(Nc2ccc(N3CCC(C(=O)N(C)C)CC3)cc2)n1]

In a similar manner as described in Example D-269, 5-[(2R,3R)-3-(4-cyclopropylbenzamido)-2-methylpiperidin-1-yl]-3-({4-[4-(dimethylcarbamoyl)piperidin-1-yl]phenyl}amino)pyrazine-2-carboxamide (D-320) was prepared using 1-(4-aminophenyl)-N,N-dimethylpiperidine-4-carboxamide. MS found for C35H44N8O3 as (M+H)⁺ 625.3.

¹H NMR (500 MHz, DMSO) δ 10.95 (s, 1H), 8.33 (d, J=7.43 Hz, 1H), 7.84 (d, J=8.22 Hz, 2H), 7.70 (br. s., 1H), 7.59 (s, 1H), 7.45 (d, J=9.00 Hz, 2H), 7.27 (d, J=1.96 Hz, 1H), 7.17 (d, J=8.22 Hz, 2H), 6.81 (d, J=9.00 Hz, 2H), 5.49-4.84 (m, 1H), 4.30-3.94 (m, 2H), 3.56-3.38 (m, 2H), 3.18-2.79 (m, 7H), 2.73-2.64 (m, 1H), 2.62-2.51 (m, 2H), 2.02-1.52 (m, 9H), 1.10-0.98 (m, 5H), 0.77-0.70 (m, 2H).

### Example D-321: Synthesis of 5-[(2R,3R)-3-(4-cyclopropylbenzamido)-2-methylpiperidin-1-yl]-3-({1-[(4,4-difluorocyclohexyl)methyl]-1H-pyrazol-4-yl}amino)pyrazine-2-carboxamide (D-321)

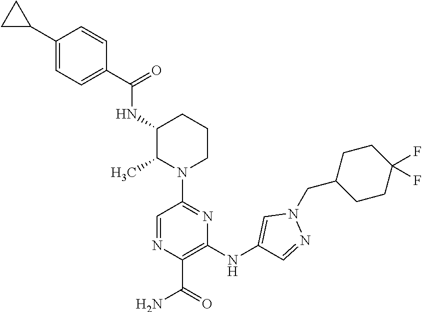 [SMILES: C[C@@H]1[C@H](NC(=O)c2ccc(C3CC3)cc2)CCCN1c1cnc(C(N)=O)c(Nc2cnn(CC3CCC(F)(F)CC3)c2)n1]

In a similar manner as described in Example D-319, 5-[(2R,3R)-3-(4-cyclopropylbenzamido)-2-methylpiperidin-1-yl]-3-({1-[(4,4-difluorocyclohexyl)methyl]-1H-pyrazol-4-yl}amino)pyrazine-2-carboxamide (D-321) was prepared using 1-[(4,4-difluorocyclohexyl)methyl]-1H-pyrazol-4-amine. MS found for C31H38F2N8O2 as (M+H)⁺ 593.3.

¹H NMR (500 MHz, DMSO) δ 10.86 (s, 1H), 8.40 (d, J=7.02 Hz, 1H), 7.82 (d, J=8.33 Hz, 2H), 7.68 (br. s., 1H), 7.57 (s, 1H), 7.45 (s, 1H), 7.27 (br. s., 1H), 7.17 (d, J=8.55 Hz, 2H), 5.13-5.61 (m, 1H), 4.22-3.97 (m, 2H), 3.94-3.66 (m, 2H), 3.10 (t, J=12.06 Hz, 1H), 1.09 (d, J=6.80 Hz, 3H), 1.05-0.99 (m, 2H), 0.76-0.70 (m, 2H), 2.05-0.65 (m, 14H).

### Example E-1. 3-((2R,3R)-3-acrylamido-2-methylpiperidin-1-yl)-5-((4-(1-cyclopropylpiperidin-4-yl)phenyl)amino)-1,2,4-triazine-6-carboxamide

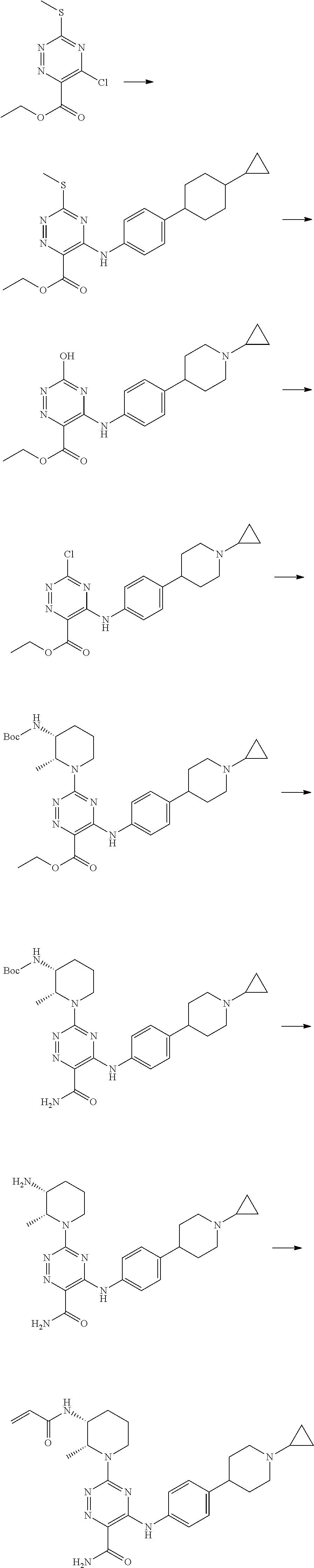

Ethyl 5-chloro-3-(methylthio)-1,2,4-triazine-6-carboxylate (490 mg, 2.10 mmol) was dissolved in 10 mL dry acetonitrile. To it were added 4-(1-cyclopropylpiperidin-4-yl)aniline (500 mg, 2.31 mmol) and then DIEA (diisopropylethylamine, 480 μL, 2.73 mmol) dropwise. The mixture was stirred at RT for 2 hours, concentrated in vacuo and subjected to flash column to isolate ethyl 5-((4-(1-cyclopropylpiperidin-4-yl)phenyl)amino)-3-(methylthio)-1,2,4-triazine-6-carboxylate (854 mg, 98%) using 0 to 6% MeOH in DCM. It was dissolved in 20 mL dioxane. To it were added triethylamine (2 mL), oxone (5.09 g, 8.27 mmol) and water (5 mL). The mixture was stirred at RT for 2 hours. It was diluted with water (20 mL) and extracted using chloroform 6 times. The chloroform extracts were combined, dried over MgSO₄, concentrated and subjected to flash column using 0 to 30% MeOH in DCM to isolate ethyl 5-((4-(1-cyclopropylpiperidin-4-yl)phenyl)amino)-3-hydroxy-1,2,4-triazine-6-carboxylate (concentrated and fully pumped to drive out all the methanol). It was then treated with POCl₃ (20 mL) at 95° C. for 3 hours. The mixture was concentrated in vacuo and quenched with saturated NaHCO₃ solution. It was extracted with chloroform 4 times. All the chloroform extracts were combined, dried over MgSO₄, and pumped to dryness to give crude ethyl 3-chloro-5-((4-(1-cyclopropylpiperidin-4-yl)phenyl)amino)-1,2,4-triazine-6-carboxylate. It was dissolved in 8 mL dry NMP. To it were added tert-butyl ((2R,3R)-2-methylpiperidin-3-yl)carbamate (320 mg, 1.5 mmol) and DIEA (350 μL, 2.0 mmol). The mixture was stirred at 90° C. for 2 hours. It was cooled to RT, diluted with ethyl acetate, washed with water 3 times, dried over MgSO₄, concentrated and subjected to flash column using 0 to 6% MeOH in DCM to isolate ethyl 3-((2R,3R)-3-((tert-butoxycarbonyl)amino)-2-methylpiperidin-1-yl)-5-((4-(1-cyclopropylpiperidin-4-yl)phenyl)amino)-1,2,4-triazine-6-carboxylate. It was dissolved in 8 mL “7N ammonia in methanol” and stirred at RT for overnight with a rubber septum to seal the flask. The mixture was concentrated in vacuo to dryness to give tert-butyl ((2R,3R)-1-(6-carbamoyl-5-((4-(1-cyclopropylpiperidin-4-yl)phenyl)amino)-1,2,4-triazin-3-yl)-2-methylpiperidin-3-yl)carbamate. It was then treated with 5 mL TFA at RT for 30 min and subjected to reverse phase preparative HPLC to isolate 3-((2R,3R)-3-amino-2-methylpiperidin-1-yl)-5-((4-(1-cyclopropylpiperidin-4-yl)phenyl)amino)-1,2,4-triazine-6-carboxamide as HCl salt (75 mg). MS found for C24H34N8O as (M+H)⁺ 451.2.

3-((2R,3R)-3-Amino-2-methylpiperidin-1-yl)-5-((4-(1-cyclopropylpiperidin-4-yl)phenyl)amino)-1,2,4-triazine-6-carboxamide HCl salt (34 mg, 0.07 mmol) was dissolved in 4 mL NMP and stirred in ice bath. To it were added DIEA (100 μL, 0.56 mmol) and then acryloyl chloride (8.5 μL, 0.105 mmol). The reaction was complete in less than 5 minutes, quenched with TFA (0.5 mL) and directly subjected to reverse phase preparative HPLC to isolate the title compound, 3-((2R,3R)-3-acrylamido-2-methylpiperidin-1-yl)-5-((4-(1-cyclopropylpiperidin-4-yl)phenyl)amino)-1,2,4-triazine-6-carboxamide (23 mg). MS found for C27H36N8O2 as (M+H)⁺ 505.2 and (M−H)⁻ 503.2.

Using synthetic schemes similar to what shown above for Example E-1, the following compounds have been prepared:

TABLE 5
Additional Compounds of Formula (A-I)

| | | LC-MS | | |
|---|---|---|---|---|
| Cmpd | | (ESI): | | |
| # | Structure | m/z | Name | Procedure |
| E-2 | 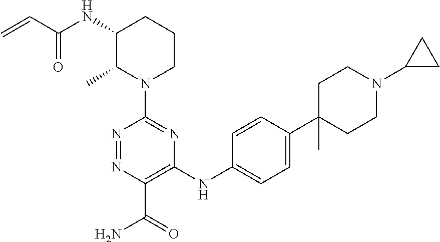 [SMILES: C=CC(=O)N[C@@H]1CCCN(c2nnc(C(N)=O)c(Nc3ccc(C4(C)CCN(C5CC5)CC4)cc3)n2)[C@@H]1C] | 519.3 | 3-((2R,3R)-3- acrylamido-2- methylpiperidin-1-yl)- 5-((4-(1-cyclopropyl- 4-methylpiperidin-4- yl)phenyl)amino)- 1,2,4-triazine-6- carboxamide | Similar to Example E-1 |
| E-3 | 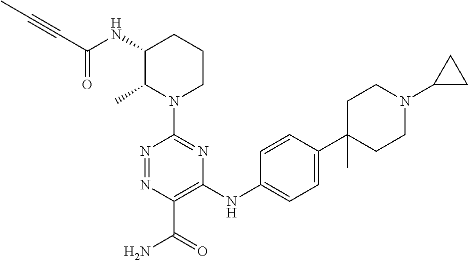 [SMILES: CC#CC(=O)N[C@@H]1CCCN(c2nnc(C(N)=O)c(Nc3ccc(C4(C)CCN(C5CC5)CC4)cc3)n2)[C@@H]1C] | 531.2 | 3-((2R,3R)-3-(but-2- ynamido)-2- methylpiperidin-1-yl)- 5-((4-(1-cyclopropyl)- 4-methylpiperidin-4- yl)phenyl)amino)- 1,2,4-triazine-6- carboxamide | Similar to Example E-1 |
| E-4 | 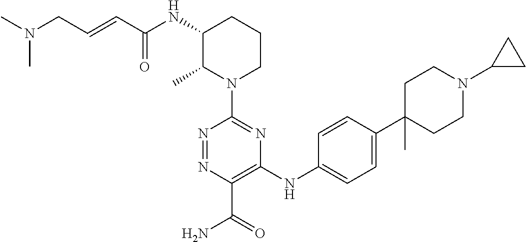 [SMILES: C[C@@H]1[C@H](NC(=O)/C=C/CN(C)C)CCCN1c1nnc(C(N)=O)c(Nc2ccc(C3(C)CCN(C4CC4)CC3)cc2)n1] | 576.2 | 5-((4-(1-cyclopropyl- 4-methylpiperidin-4- yl)phenyl)amino)-3- ((2R,3R)-3-((E)-4- (dimethylamino)but- 2-enamido)-2- methylpiperidin-1-yl)- 1,2,4-triazine-6- carboxamide | Similar to Example E-1 |
| E-5 | 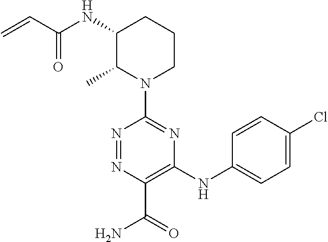 [SMILES: C=CC(=O)N[C@@H]1CCCN(c2nnc(C(N)=O)c(Nc3ccc(Cl)cc3)n2)[C@@H]1C] | 416.0 | 3-((2R,3R)-3- acrylamido-2- methylpiperidin-1-yl)- 5-((4- chlorophenyl)amino)- 1,2,4-triazine-6- carboxamide | Similar to Example E-1 |
| E-6 | 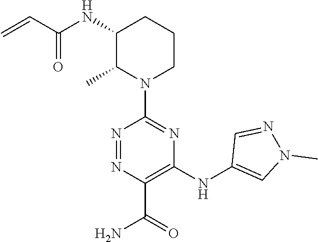 [SMILES: C=CC(=O)N[C@@H]1CCCN(c2nnc(C(N)=O)c(Nc3cnn(C)c3)n2)[C@@H]1C] | 386.1 | 3-((2R,3R)-3- acrylamido-2- methylpiperidin-1-yl)- 5-((1-methyl-1H- pyrazol-4-yl)amino)- 1,2,4-triazine-6- carboxamide | Similar to Example E-1 |

### Example E-7. 3-(((2R,3R)-1-acryloyl-2-methylpiperidin-3-yl)amino)-5-((4-chlorophenyl)amino)-1,2,4-triazine-6-carboxamide

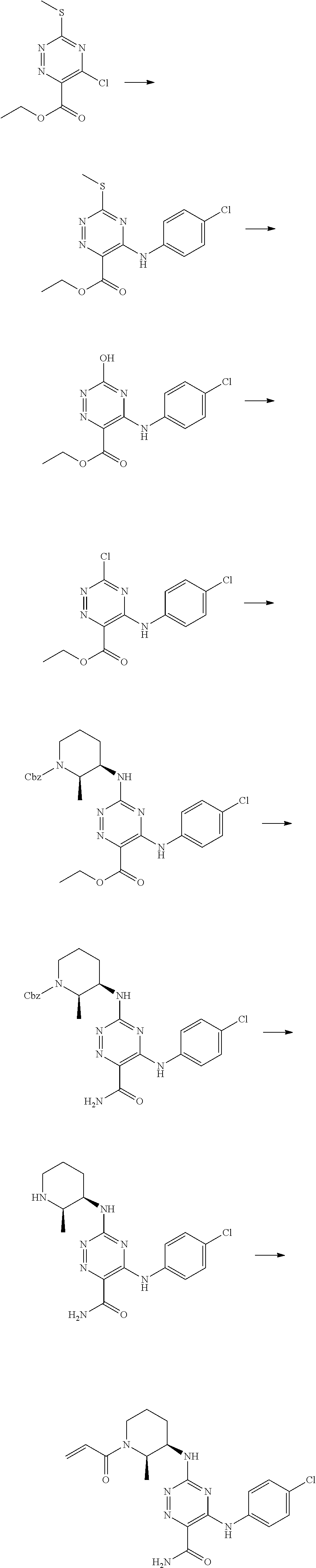

Ethyl 5-chloro-3-(methylthio)-1,2,4-triazine-6-carboxylate (490 mg, 2.10 mmol) was dissolved in 10 mL dry acetonitrile. To it were added 4-chloroaniline (300 mg, 2.31 mmol) and then DIEA (diisopropylethylamine, 480 μL, 2.73 mmol) dropwise. The mixture was stirred at RT for 2 hours, concentrated in vacuo and subjected to flash column to isolate ethyl 5-((4-chlorophenyl)amino)-3-(methylthio)-1,2,4-triazine-6-carboxylate in quantitative yield. Ethyl 5-((4-chlorophenyl)amino)-3-(methylthio)-1,2,4-triazine-6-carboxylate (650 mg, 2.00 mmol) was dissolved in 10 mL wet DCM. MCPBA (70% strength, 2.47 grams, 10.0 mmol) was added and the mixture was stirred at RT for overnight. It was diluted with 40 mL MTBE, and the mixture was vigorously triturated. It was filtered through a ChemGlass OP-6602-12 filter. The solid cake was carefully washed with MTBE till no much meta-chlorobenzoic acid present in the solid. The solid was the desired ethyl 5-((4-chlorophenyl)amino)-3-hydroxy-1,2,4-triazine-6-carboxylate (620 mg, quantitative yield), and dried in vacuo. It was treated with 20 mL POCl₃ at 95° C. for 3 hours. The mixture was concentrated in vacuo, quenched with saturated NaHCO₃ solution, extracted with chloroform three times. The chloroform extracts were combined, dried over MgSO₄, pumped to dryness, and subjected to flash column using 0 to 15% EtOAc in DCM to isolate ethyl 3-chloro-5-((4-chlorophenyl)amino)-1,2,4-triazine-6-carboxylate (329 mg, 53%). This compound (135 mg, 0.43 mmol) was dissolved in 8 mL NMP. To it were added commercial benzyl (2R,3R)-3-amino-2-methylpiperidine-1-carboxylate (213 mg, 0.86 mmol) and DIEA (230 μL, 1.29 mmol). The mixture was stirred at 90° C. for 1 hour, cooled to RT, diluted with EtOAc, washed with water three times, dried, concentrated and subjected to flash column with 0 to 5% MeOH in DCM to isolate ethyl 3-(((2R,3R)-1-((benzyloxy)carbonyl)-2-methylpiperidin-3-yl)amino)-5-((4-chlorophenyl)amino)-1,2,4-triazine-6-carboxylate. It was treated with 8 mL “7N ammonia in methanol” and stirred as a slurry at RT for overnight with a rubber septum to seal the flask. The mixture was concentrated in vacuo to dryness to give benzyl (2R,3R)-3-((6-carbamoyl-5-((4-chlorophenyl)amino)-1,2,4-triazin-3-yl)amino)-2-methylpiperidine-1-carboxylate. It was then treated with DCM (8 mL)/TFA (4 mL)/TfOH (0.4 mL) at RT for 4 hours to cleave the Cbz group. The mixture was concentrated in vacuo and directly subjected to reverse phase preparative HPLC to isolate 5-((4-chlorophenyl)amino)-3-(((2R,3R)-2-methylpiperidin-3-yl)amino)-1,2,4-triazine-6-carboxamide as HCl salt (174 mg). MS found for C16H20ClN7O as (M+H)⁺ 362.0 and (M−H)⁻ 360.0.

5-((4-Chlorophenyl)amino)-3-(((2R,3R)-2-methylpiperidin-3-yl)amino)-1,2,4-triazine-6-carboxamide HCl salt (55 mg, 0.14 mmol) was dissolved in 4 mL NMP and stirred in ice bath. To it were added DIEA (200 μL, 1.12 mmol) and then acryloyl chloride (17 μL, 0.21 mmol). The reaction was complete in less than 5 minutes, quenched with TFA (0.5 mL) and directly subjected to reverse phase preparative HPLC to isolate the title compound, 3-(((2R,3R)-1-acryloyl-2-methylpiperidin-3-yl)amino)-5-((4-chlorophenyl)amino)-1,2,4-triazine-6-carboxamide (38 mg). MS found for C19H22ClN7O2 as (M+H)⁺ 416.0 and (M−H)⁻ 414.0.

Using synthetic schemes similar to what shown above for Example E-1, the following compounds have been prepared:

TABLE 6
Additional Compounds of Formula (C-I)

| | | LC-MS | | |
|---|---|---|---|---|
| Cmpd | | (ESI): | | |
| # | Structure | m/z | Name | Procedure |
| E-8 | 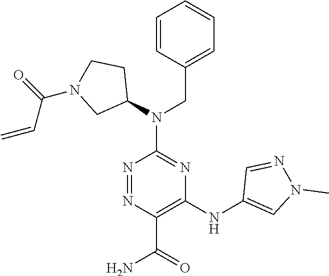 [SMILES: C=CC(=O)N1CC[C@@H](N(Cc2ccccc2)c2nnc(C(N)=O)c(Nc3cnn(C)c3)n2)C1] | 448.1 | (R)-3-((1- acryloylpyrrolidin-3- yl)(benzyl)amino)-5- ((1-methyl-1H- pyrazol-4-yl)amino)- 1,2,4-triazine-6- carboxamide | Similar to Example E-7 |
| E-9 | 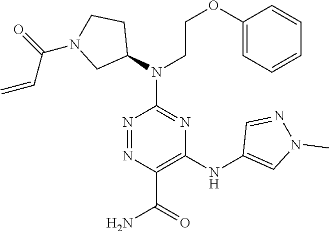 [SMILES: C=CC(=O)N1CC[C@@H](N(CCOc2ccccc2)c2nnc(C(N)=O)c(Nc3cnn(C)c3)n2)C1] | 478.2 | (R)-3-((1- acryloylpyrrolidin-3- yl)(2- phenoxyethyl)amino)- 5-((1-methyl-1H- pyrazol-4-yl)amino)- 1,2,4-triazine-6- carboxamide | Similar to Example E-7 |

### Example E-10. 3-((3R,3'S,4'S)-3-(3-chloro-5-(trifluoromethyl)phenylamino)-4′-hydroxy-2-oxo-1,3′-bipiperidin-1′-yl)-5-(3-methylisothiazol-5-ylamino)-1,2,4-triazine-6-carboxamide

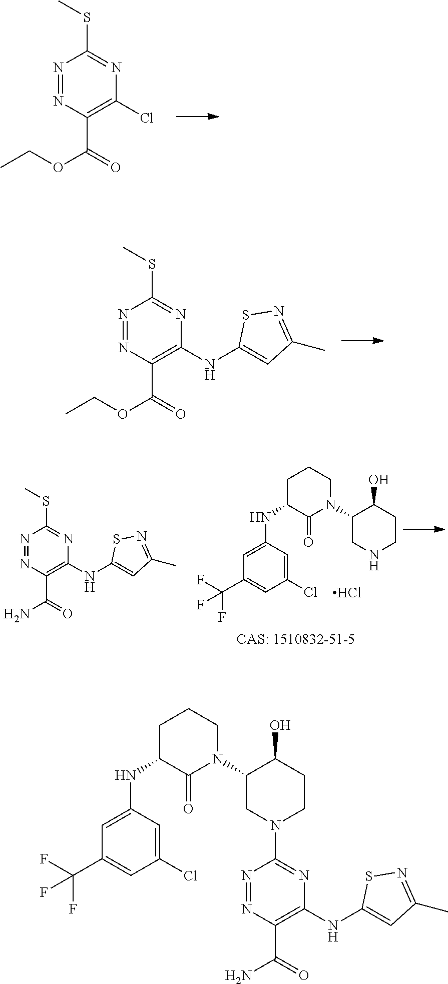

Ethyl 5-chloro-3-(methylthio)-1,2,4-triazine-6-carboxylate (2.02 g, 8.63 mmol) was mixed with 5-amino-3-methylisothiazole HCl (2.60 g, 17.2 mmol), powder cesium carbonate (14.1 g, 43.2 mmol), Pd₂(dba)₃ (1.58 g, 3.44 mmol), XantPhos (2.00 g, 3.44 mmol) in 100 mL toluene. The mixture was degassed using nitrogen stream for 5 min and refluxed gently under nitrogen atmosphere for overnight. The mixture was filtered through a ChemGlass OP-6602-12 filter, and the filtrate was concentrated and subjected to flash column using 0-80% EtOAc in hexane to isolate ethyl 5-((3-methylisothiazol-5-yl)amino)-3-(methylthio)-1,2,4-triazine-6-carboxylate (1.68 g, 62%). It was treated with 30 mL “7N ammonia in methanol” for overnight to get 5-((3-methylisothiazol-5-yl)amino)-3-(methylthio)-1,2,4-triazine-6-carboxamide cleanly, isolated by simple concentration in vacuo and pumped to dryness.

5-((3-Methylisothiazol-5-yl)amino)-3-(methylthio)-1,2,4-triazine-6-carboxamide (100 mg, 0.35 mmol) was dissolved in 4 mL dry NMP. To it was added MCPBA (70% strength, 255 mg, 1.06 mmol). The mixture was stirred at RT for 30 min to get a mixture of sulfone and sulfoxide. To it were added DIEA (610 μL, 3.5 mmol) and (3R,3′S,4′S)-3-((3-chloro-5-(trifluoromethyl)phenyl)amino)-4′-hydroxy-[1,3′-bipiperidin]-2-one (CAS: 1510832-51-5) hydrochloride (205 mg, 0.52 mmol). The mixture was sent to 90° C. to store for 1.5 hour. It was cooled to RT, quenched with 1 mL TFA, and directly subjected to reverse phase preparative HPLC to isolate the title compound, 3-((3R,3′S,4′S)-3-(3-chloro-5-(trifluoromethyl)phenylamino)-4′-hydroxy-2-oxo-1,3′-bipiperidin-1′-yl)-5-(3-methylisothiazol-5-ylamino)-1,2,4-triazine-6-carboxamide (64 mg, with MS found for C25H27ClF3N9O3S as (M+H)⁺ 626.1 and (M−H)⁻ 624.0) and leftover (3R,3'S,4'S)-3-((3-chloro-5-(trifluoromethyl)phenyl)amino)-4′-hydroxy-[1,3′-bipiperidin]-2-one hydrochloride (69 mg).

### Example E-11. (R)-3-(1-acryloylpiperidin-3-ylamino)-5(3-methylisothiazol-5-ylamino)-1,2,4-triazine-6-carboxamide

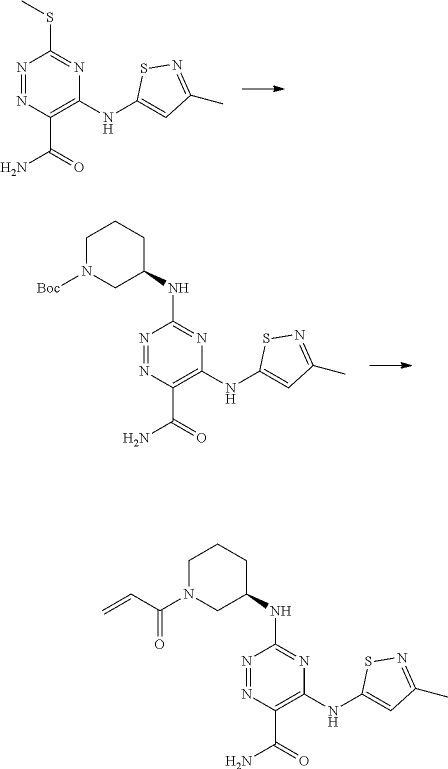

5-((3-Methylisothiazol-5-yl)amino)-3-(methylthio)-1,2,4-triazine-6-carboxamide (210 mg, 0.74 mmol) was dissolved in 8 mL dry NMP. To it was added MCPBA (70% strength, 550 mg, 2.23 mmol). The mixture was stirred at RT for 45 min to get a mixture of sulfone and sulfoxide. To it were added DIEA (1290 μL, 7.4 mmol) and tert-butyl (R)-3-aminopiperidine-1-carboxylate (300 mg, 1.5 mmol). The mixture was stirred at 85° C. for 1 hour, diluted with EtOAc, washed with water three times, concentrated and subjected to flash column using 0 to 3.5% MeOH in DCM to isolate tert-butyl (R)-3-((6-carbamoyl-5-((3-methylisothiazol-5-yl)amino)-1,2,4-triazin-3-yl)amino)piperidine-1-carboxylate (158 mg, 49%). It was dissolved in 10 mL MeOH and treated with 5 mL “4N HCl in dioxane” for 1 hour. The mixture was concentrated in vacuo to dryness. It was dissolved in 4 mL DMF and stirred in ice bath. To it were added DIEA (380 μL, 2.16 mmol) and then acryloyl chloride (35 μL, 0.43 mmol). The reaction was allowed for 5 min and quenched with 0.5 mL TFA. The mixture was then subjected to reverse phase preparative HPLC to isolate the title compound, (R)-3-(1-acryloylpiperidin-3-ylamino)-5-(3-methylisothiazol-5-ylamino)-1,2,4-triazine-6-carboxamide (84 mg). MS found for C16H20N8O2S as (M+H)⁺ 389.0 and (M−H)⁻ 386.9.

Using synthetic schemes similar to what shown above for Example E-11, the following compounds have been prepared:

TABLE 7
Additional Compounds of Formula (C-I)

| | | LC-MS | | |
|---|---|---|---|---|
| Cmpd | | (ESI): | | |
| # | Structure | m/z | Name | Procedure |
| E-12 | 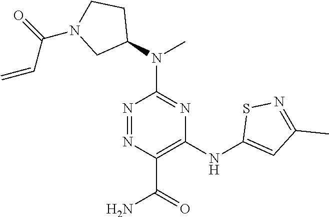 [SMILES: C=CC(=O)N1CC[C@@H](N(C)c2nnc(C(N)=O)c(Nc3cc(C)ns3)n2)C1] | 389.0 | (R)-3-((1- acryloylpyrrolidin-3- yl)(methyl)amino)-5- (3-methylisothiazol-5- ylamino)-1,2,4- triazine-6- carboxamide | Similar to Example E-11 |
| E-13 | 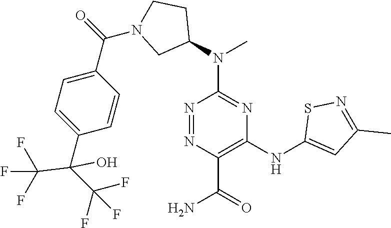 [SMILES: Cc1cc(Nc2nc(N(C)[C@@H]3CCN(C(=O)c4ccc(C(O)(C(F)(F)F)C(F)(F)F)cc4)C3)nnc2C(N)=O)sn1] | 605.1 | (R)-3-((1-(4- (1,1,1,3,3,3- hexafluoro-2- hydroxypropan-2- yl)benzoyl)pyrrolidin- 3-yl)(methyl)amino)- 5-(3-methylisothiazol- 5-ylamino)-1,2,4- triazine-6- carboxamide | Similar to Example E-11 |
| E-14 | 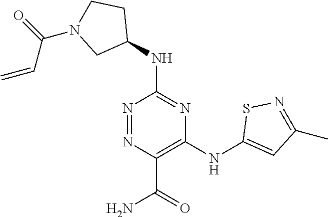 [SMILES: C=CC(=O)N1CC[C@@H](Nc2nnc(C(N)=O)c(Nc3cc(C)ns3)n2)C1] | 375.0 | (R)-3-(1- acryloylpyrrolidin-3- ylamino)-5-(3- methylisothiazol-5- ylamino)-1,2,4- triazine-6- carboxamide | Similar to Example E-11 |
| E-15 | 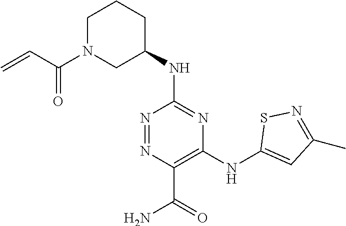 [SMILES: C=CC(=O)N1CCC[C@@H](Nc2nnc(C(N)=O)c(Nc3cc(C)ns3)n2)C1] | 389.3 | (R)-3-(1- acryloylpiperidin-3- ylamino)-5-(3- methylisothiazol-5- ylamino)-1,2,4- triazine-6- carboxamide | Similar to Example E-11 |

### Example E-16. (R)-3-((1-acryloylpyrrolidin-3-yl)(methyl)amino)-5-(4-(1,1,1,3,3,3-hexafluoro-2-hydroxypropan-2-yl)phenylamino)-1,2,4-triazine-6-carboxamide

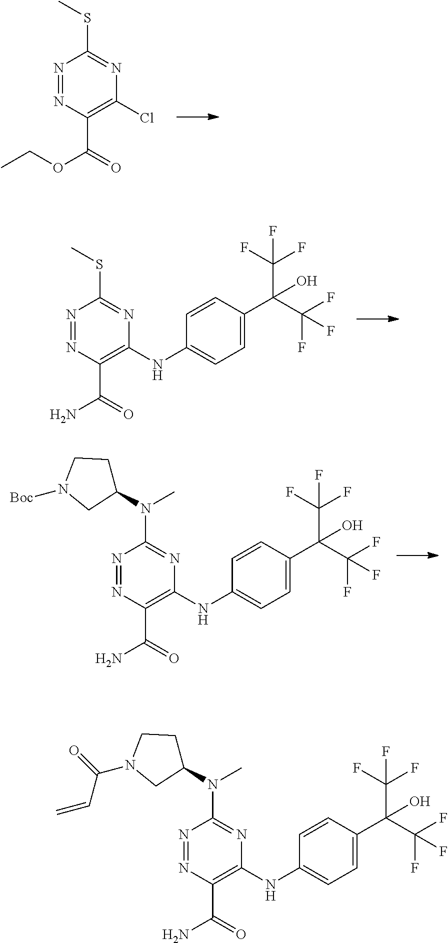

Ethyl 5-chloro-3-(methylthio)-1,2,4-triazine-6-carboxylate (620 mg, 2.66 mmol) was dissolved in 30 mL dry acetonitrile. To it were added commercial 2-(4-aminophenyl)-1,1,1,3,3,3-hexafluoropropan-2-ol (1.04 g, 4.00 mmol) and then DIEA (925 μL, 5.32 mmol) dropwise. The mixture was stirred for 2 hours at RT. To it was then added “7N ammonia in methanol” (7.6 mL, 53.2 mmol). The flask was sealed using a septum and the mixture was stirred for overnight. It was concentrated in vacuo and subjected to flash column using 0 to 10% MeOH in DCM to isolate 5-((4-(1,1,1,3,3,3-hexafluoro-2-hydroxypropan-2-yl)phenyl)amino)-3-(methylthio)-1,2,4-triazine-6-carboxamide in quantitative yield. This compound (100 mg, 0.23 mmol) was dissolved in 5 mL dry NMP. To it was added MCPBA (70% strength, 210 mg, 0.94 mmol). The mixture was stirred at RT for 30 min to produce sulfoxide and sulfone. To it were added DIEA (320 μL, 1.84 mmol) and then tert-butyl (R)-3-(methylamino)pyrrolidine-1-carboxylate (92 mg, 0.46 mmol). The mixture was sent to 90° C. for 1 hour. It was cooled to RT, diluted with EtOAc, washed with water twice, concentrated in vacuo and subjected to flash column using 0 to 100% EtOAc in DCM to isolate tert-butyl (R)-3-((6-carbamoyl-5-((4-(1,1,1,3,3,3-hexafluoro-2-hydroxypropan-2-yl)phenyl)amino)-1,2,4-triazin-3-yl)(methyl)amino)pyrrolidine-1-carboxylate (54 mg, 41%). It was dissolved in 6 mL methanol and treated with 3 mL “4N HCl in dixoane” for 30 min. The mixture was concentrated in vacuo to dryness. It was then dissolved in 4 mL DMF and stirred in ice bath. To it were added DIEA (130 μL, 0.74 mmol) and then acryloyl chloride (15.2 μL, 0.19 mmol). The reaction was allowed for 10 min and quenched with 0.3 mL TFA. The mixture was then subjected to reverse phase preparative HPLC to isolate the title compound, (R)-3-((1-acryloylpyrrolidin-3-yl)(methyl)amino)-5-(4-(1,1,1,3,3,3-hexafluoro-2-hydroxypropan-2-yl)phenylamino)-1,2,4-triazine-6-carboxamide (17 mg). MS found for C21H21F6N7O3 as (M+H)⁺ 534.1 and (M−H)⁻ 532.1.

Using synthetic schemes similar to what shown above for Example E-16, the following compounds have been prepared:

TABLE 8
Additional Compounds of Formula (C-I)

| | | LC-MS | | |
|---|---|---|---|---|
| Cmpd | | (ESI): | | |
| # | Structure | m/z | Name | Procedure |
| E-17 | 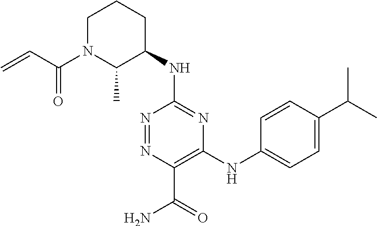 [SMILES: C=CC(=O)N1CCC[C@@H](Nc2nnc(C(N)=O)c(Nc3ccc(C(C)C)cc3)n2)[C@@H]1C] | 424.3 | 3-((2S,3R)-1-acryloyl- 2-methylpiperidin-3- ylamino)-5-(4- isopropylphenylamino)- 1,2,4-triazine-6- carboxamide | Similar to Example E-16 |
| E-18 | 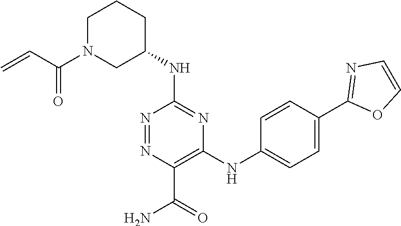 [SMILES: C=CC(=O)N1CCC[C@H](Nc2nnc(C(N)=O)c(Nc3ccc(-c4ncco4)cc3)n2)C1] | 435.2 | (S)-3-(1- acryloylpiperidin-3- ylamino)-5-(4-(oxazol- 2-yl)phenylamino)- 1,2,4-triazine-6- carboxamide | Similar to Example E-16 |
| E-19 | 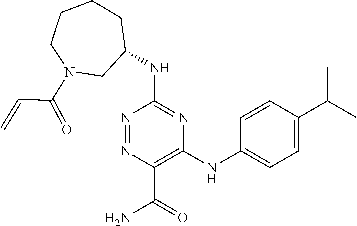 [SMILES: C=CC(=O)N1CCCC[C@H](Nc2nnc(C(N)=O)c(Nc3ccc(C(C)C)cc3)n2)C1] | 424.4 | (S)-3-(1- acryloylazepan-3- ylamino)-5-(4- isopropylphenylamino)- 1,2,4-triazine-6- carboxamide | Similar to Example E-16 |
| E-20 | 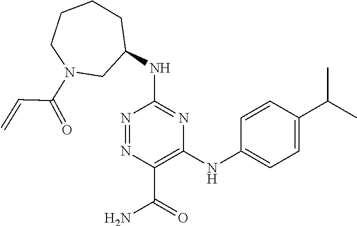 [SMILES: C=CC(=O)N1CCCC[C@@H](Nc2nnc(C(N)=O)c(Nc3ccc(C(C)C)cc3)n2)C1] | 424.2 | (R)-3-(1- acryloylazepan-3- ylamino)-5-(4- isopropylphenylamino)- 1,2,4-triazine-6- carboxamide | Similar to Example E-16 |
| E-21 | 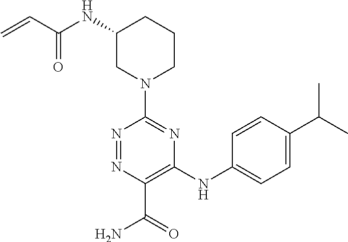 [SMILES: C=CC(=O)N[C@@H]1CCCN(c2nnc(C(N)=O)c(Nc3ccc(C(C)C)cc3)n2)C1] | 410.3 | (R)-3-(3- acrylamidopiperidin-1- yl)-5-(4- isopropylphenylamino)- 1,2,4-triazine-6- carboxamide | Similar to Example E-16 |
| E-22 | 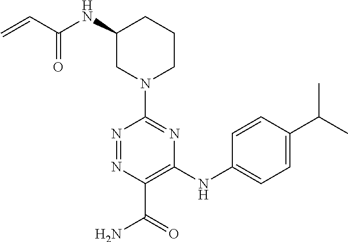 [SMILES: C=CC(=O)N[C@H]1CCCN(c2nnc(C(N)=O)c(Nc3ccc(C(C)C)cc3)n2)C1] | 410.4 | (S)-3-(3- acrylamidopiperidin-1- yl)-5-(4- isopropylphenylamino)- 1,2,4-triazine-6- carboxamide | Similar to Example E-16 |
| E-23 | 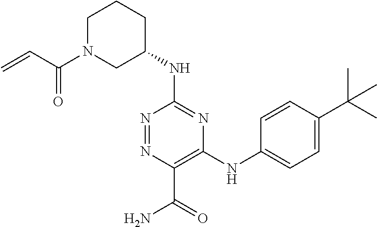 [SMILES: C=CC(=O)N1CCC[C@H](Nc2nnc(C(N)=O)c(Nc3ccc(C(C)(C)C)cc3)n2)C1] | 424.4 | (S)-3-(1- acryloylpiperidin-3- ylamino)-5-(4-tert- butylphenylamino)- 1,2,4-triazine-6- carboxamide | Similar to Example E-16 |
| E-24 | 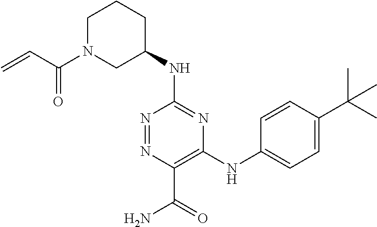 [SMILES: C=CC(=O)N1CCC[C@@H](Nc2nnc(C(N)=O)c(Nc3ccc(C(C)(C)C)cc3)n2)C1] | 424.4 | (R)-3-(1- acryloylpiperidin-3- ylamino)-5-(4-tert- butylphenylamino)- 1,2,4-triazine-6- carboxamide | Similar to Example E-16 |
| E-25 | 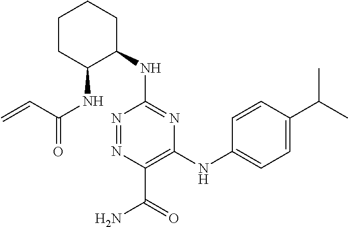 [SMILES: C=CC(=O)N[C@H]1CCCC[C@H]1Nc1nnc(C(N)=O)c(Nc2ccc(C(C)C)cc2)n1] | 424.4 | 3-((1R,2S)-2- acrylamidocyclohexyl- amino)-5-(4- isopropylphenylamino)- 1,2,4-triazine-6- carboxamide | Similar to Example E-16 |
| E-26 | 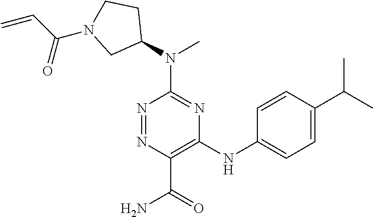 [SMILES: C=CC(=O)N1CC[C@@H](N(C)c2nnc(C(N)=O)c(Nc3ccc(C(C)C)cc3)n2)C1] | 410.4 | (R)-3-((1- acryloylpyrrolidin-3- yl)(methyl)amino)-5- (4- isopropylphenylamino)- 1,2,4-triazine-6- carboxamide | Similar to Example E-16 |
| E-27 | 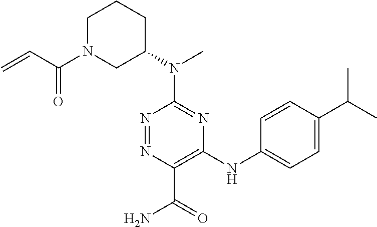 [SMILES: C=CC(=O)N1CCC[C@H](N(C)c2nnc(C(N)=O)c(Nc3ccc(C(C)C)cc3)n2)C1] | 424.3 | (S)-3-((1- acryloylpiperidin-3- yl)(methyl)amino)-5- (4- isopropylphenylamino)- 1,2,4-triazine-6- carboxamide | Similar to Example E-16 |
| E-28 | 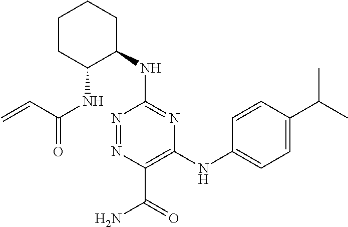 [SMILES: C=CC(=O)N[C@@H]1CCCC[C@H]1Nc1nnc(C(N)=O)c(Nc2ccc(C(C)C)cc2)n1] | 424.4 | 3-((1R,2R)-2- acrylamidocyclo- hexylamino)-5-(4- isopropylphenylamino)- 1,2,4-triazine-6- carboxamide | Similar to Example E-16 |
| E-29 | 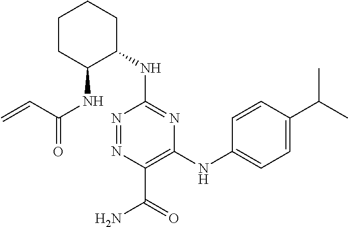 [SMILES: C=CC(=O)N[C@H]1CCCC[C@@H]1Nc1nnc(C(N)=O)c(Nc2ccc(C(C)C)cc2)n1] | 424.4 | 3-((1S,2S)-2- acrylamidocyclohexyl- amino)-5-(4- isopropylphenylamino)- 1,2,4-triazine-6- carboxamide | Similar to Example E-16 |
| E-32 | 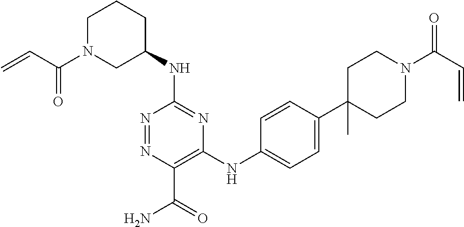 [SMILES: C=CC(=O)N1CCC(C)(c2ccc(Nc3nc(N[C@@H]4CCCN(C(=O)C=C)C4)nnc3C(N)=O)cc2)CC1] | 519.4 | (R)-5-(4-(1-acryloyl-4- methylpiperidin-4- yl)phenylamino)-3-(1- acryloylpiperidin-3- ylamino)-1,2,4-triazine- 6-carboxamide | Similar to Example E-16 |
| E-34 | 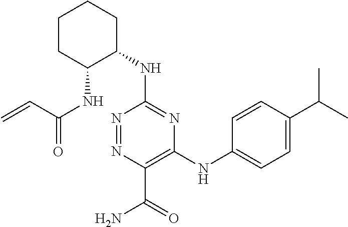 [SMILES: C=CC(=O)N[C@@H]1CCCC[C@@H]1Nc1nnc(C(N)=O)c(Nc2ccc(C(C)C)cc2)n1] | 424.4 | 3-((1S,2R)-2- acrylamidocyclohexyl- amino)-5-(4- isopropylphenylamino)- 1,2,4-triazine-6- carboxamide | Similar to Example E-16 |
| E-35 | 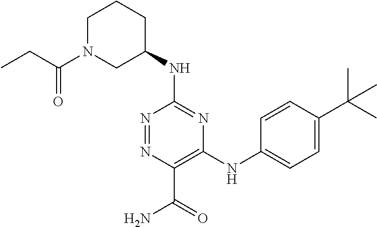 [SMILES: CCC(=O)N1CCC[C@@H](Nc2nnc(C(N)=O)c(Nc3ccc(C(C)(C)C)cc3)n2)C1] | 426.5 | (R)-5-(4-tert- butylphenylamino)-3- (1-propionylpiperidin- 3-ylamino)-1,2,4- triazine-6-carboxamide | Similar to Example E-16 |
| E-36 | 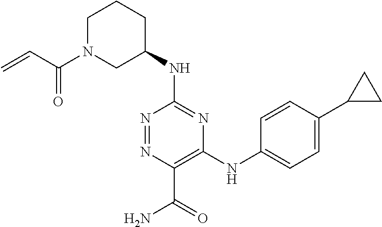 [SMILES: C=CC(=O)N1CCC[C@@H](Nc2nnc(C(N)=O)c(Nc3ccc(C4CC4)cc3)n2)C1] | 408.2 | (R)-3-(1- acryloylpiperidin-3- ylamino)-5-(4- cyclopropylphenyl- amino)-1,2,4-triazine-6- carboxamide | Similar to Example E-16 |
| E-37 | 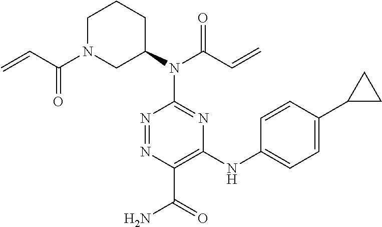 [SMILES: C=CC(=O)N1CCC[C@@H](N(C(=O)C=C)c2nnc(C(N)=O)c(Nc3ccc(C4CC4)cc3)n2)C1] | 462.4 | (R)-3-(N-(1- acryloylpiperidin-3- yl)acrylamido)-5-(4- cyclopropylphenyl- amino)-1,2,4-triazine-6- carboxamide | Similar to Example E-16 |
| E-38 | 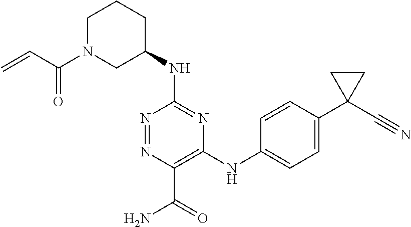 [SMILES: C=CC(=O)N1CCC[C@@H](Nc2nnc(C(N)=O)c(Nc3ccc(C4(C#N)CC4)cc3)n2)C1] | 433.1 | (R)-3-(1- acryloylpiperidin-3- ylamino)-5-(4-(1- cyanocyclopropyl) phenylamino)-1,2,4- triazine-6-carboxamide | Similar to Example E-16 |
| E-39 | 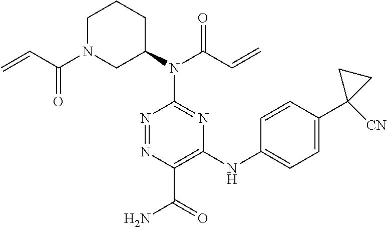 [SMILES: C=CC(=O)N1CCC[C@@H](N(C(=O)C=C)c2nnc(C(N)=O)c(Nc3ccc(C4(C#N)CC4)cc3)n2)C1] | 487.3 | (R)-3-(N-(1- acryloylpiperidin-3- yl)acrylamido)-5-(4-(1- cyanocyclopropyl) phenylamino)-1,2,4- triazine-6-carboxamide | Similar to Example E-16 |
| E-40 | 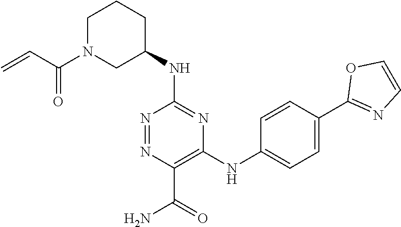 [SMILES: C=CC(=O)N1CCC[C@@H](Nc2nnc(C(N)=O)c(Nc3ccc(-c4ncco4)cc3)n2)C1] | 435.3 | (R)-3-(1- acryloylpiperidin-3- ylamino)-5-(4-(oxazol- 2-yl)phenylamino)- 1,2,4-triazine-6- carboxamide | Similar to Example E-16 |
| E-41 | 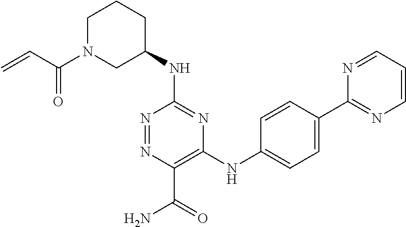 [SMILES: C=CC(=O)N1CCC[C@@H](Nc2nnc(C(N)=O)c(Nc3ccc(-c4ncccn4)cc3)n2)C1] | 446.2 | (R)-3-(1- acryloylpiperidin-3- ylamino)-5-(4- (pyrimidin-2- yl)phenylamino)-1,2,4- triazine-6-carboxamide | Similar to Example E-16 |
| E-42 | 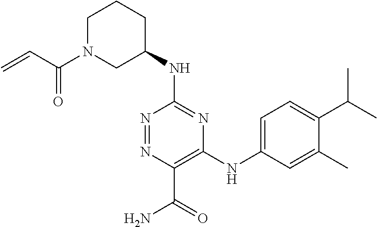 [SMILES: C=CC(=O)N1CCC[C@@H](Nc2nnc(C(N)=O)c(Nc3ccc(C(C)C)c(C)c3)n2)C1] | 424.3 | (R)-3-(1- acryloylpiperidin-3- ylamino)-5-(4- isopropyl-3- methylphenylamino)- 1,2,4-triazine-6- carboxamide | Similar to Example E-16 |
| E-43 | 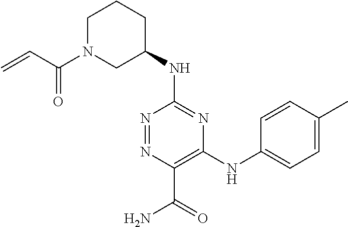 [SMILES: C=CC(=O)N1CCC[C@@H](Nc2nnc(C(N)=O)c(Nc3ccc(C)cc3)n2)C1] | 382.3 | (R)-3-(1- acryloylpiperidin-3- ylamino)-5-(p- tolylamino)-1,2,4- triazine-6-carboxamide | Similar to Example E-16 |
| E-44 | 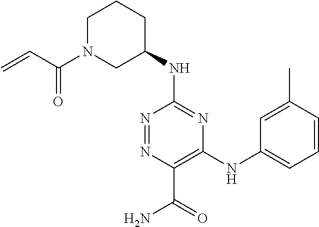 [SMILES: C=CC(=O)N1CCC[C@@H](Nc2nnc(C(N)=O)c(Nc3cccc(C)c3)n2)C1] | 382.1 | (R)-3-(1- acryloylpiperidin-3- ylamino)-5-(m- tolylamino)-1,2,4- triazine-6-carboxamide | Similar to Example E-16 |
| E-45 | 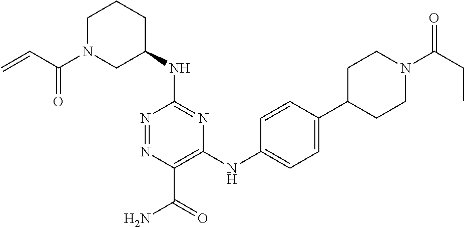 [SMILES: C=CC(=O)N1CCC[C@@H](Nc2nnc(C(N)=O)c(Nc3ccc(C4CCN(C(=O)CC)CC4)cc3)n2)C1] | 507.4 | (R)-3-(1- acryloylpiperidin-3- ylamino)-5-(4-(1- propionylpiperidin-4- yl)phenylamino)-1,2,4- triazine-6-carboxamide | Similar to Example E-16 |
| E-46 | 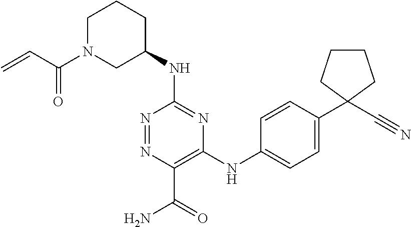 [SMILES: C=CC(=O)N1CCC[C@@H](Nc2nnc(C(N)=O)c(Nc3ccc(C4(C#N)CCCC4)cc3)n2)C1] | 461.4 | (R)-3-(1- acryloylpiperidin-3- ylamino)-5-(4-(1- cyanocyclopentyl) phenylamino)-1,2,4- triazine-6-carboxamide | Similar to Example E-16 |
| E-47 | 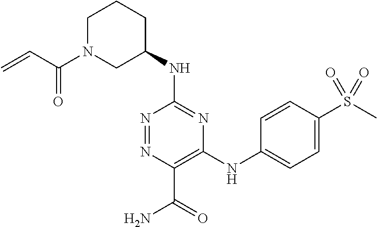 [SMILES: C=CC(=O)N1CCC[C@@H](Nc2nnc(C(N)=O)c(Nc3ccc(S(C)(=O)=O)cc3)n2)C1] | 446.1 | (R)-3-(1- acryloylpiperidin-3- ylamino)-5-(4- (methylsulfonyl)phenyl- amino)-1,2,4-triazine- 6-carboxamide | Similar to Example E-16 |
| E-48 | 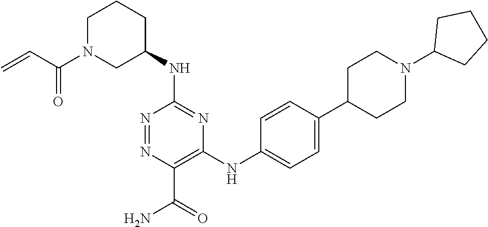 [SMILES: C=CC(=O)N1CCC[C@@H](Nc2nnc(C(N)=O)c(Nc3ccc(C4CCN(C5CCCC5)CC4)cc3)n2)C1] | 519.4 | (R)-3-(1- acryloylpiperidin-3- ylamino)-5-(4-(1- cyclopentylpiperidin-4- yl)phenylamino)-1,2,4- triazine-6-carboxamide | Similar to Example E-16 |
| E-49 | 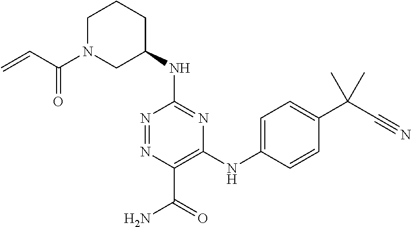 [SMILES: C=CC(=O)N1CCC[C@@H](Nc2nnc(C(N)=O)c(Nc3ccc(C(C)(C)C#N)cc3)n2)C1] | 435.3 | (R)-3-(1- acryloylpiperidin-3- ylamino)-5-(4-(2- cyanopropan-2- yl)phenylamino)-1,2,4- triazine-6-carboxamide | Similar to Example E-16 |
| E-50 | 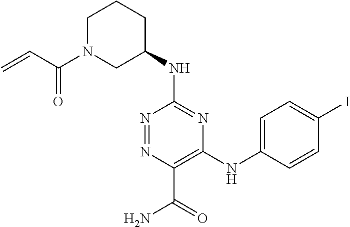 [SMILES: C=CC(=O)N1CCC[C@@H](Nc2nnc(C(N)=O)c(Nc3ccc(I)cc3)n2)C1] | 494.0 | (R)-3-(1- acryloylpiperidin-3- ylamino)-5-(4- iodophenylamino)- 1,2,4-triazine-6- carboxamide | Similar to Example E-16 |
| E-51 | 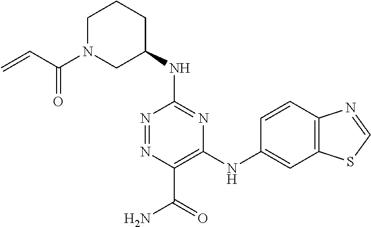 [SMILES: C=CC(=O)N1CCC[C@@H](Nc2nnc(C(N)=O)c(Nc3ccc4ncsc4c3)n2)C1] | 425.0 | (R)-3-(1- acryloylpiperidin-3- ylamino)-5- (benzo[d]thiazol-6- ylamino)-1,2,4-triazine- 6-carboxamide | Similar to Example E-16 |
| E-52 | 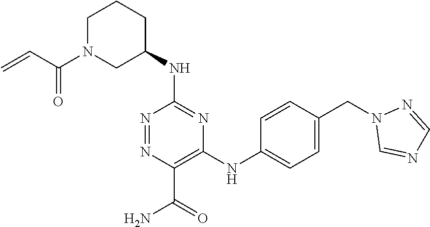 [SMILES: C=CC(=O)N1CCC[C@@H](Nc2nnc(C(N)=O)c(Nc3ccc(Cn4cncn4)cc3)n2)C1] | 449.1 | (R)-5-(4-((1H-1,2,4- triazol-1- yl)methyl)phenyl- amino)-3-(1- acryloylpiperidin-3- ylamino)-1,2,4-triazine- 6-carboxamide | Similar to Example E-16 |
| E-53 | 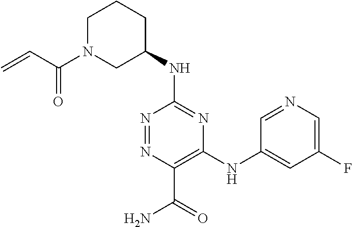 [SMILES: C=CC(=O)N1CCC[C@@H](Nc2nnc(C(N)=O)c(Nc3cncc(F)c3)n2)C1] | 387.0 | (R)-3-(1- acryloylpiperidin-3- ylamino)-5-(5- fluoropyridin-3- ylamino)-1,2,4-triazine- 6-carboxamide | Similar to Example E-16 |
| E-54 | 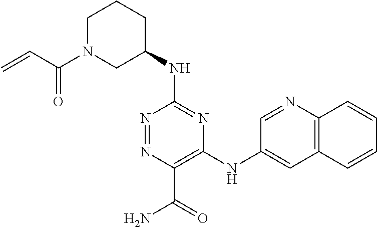 [SMILES: C=CC(=O)N1CCC[C@@H](Nc2nnc(C(N)=O)c(Nc3cnc4ccccc4c3)n2)C1] | 419.2 | (R)-3-(1- acryloylpiperidin-3- ylamino)-5-(quinolin-3- ylamino)-1,2,4-triazine- 6-carboxamide | Similar to Example E-16 |
| E-55 | 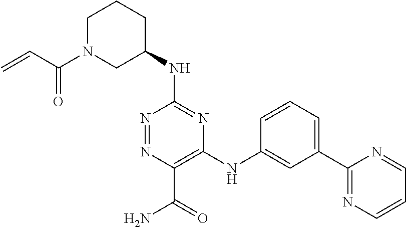 [SMILES: C=CC(=O)N1CCC[C@@H](Nc2nnc(C(N)=O)c(Nc3cccc(-c4ncccn4)c3)n2)C1] | 446.2 | (R)-3-(1- acryloylpiperidin-3- ylamino)-5-(3- (pyrimidin-2- yl)phenylamino)-1,2,4- triazine-6-carboxamide | Similar to Example E-16 |
| E-56 | 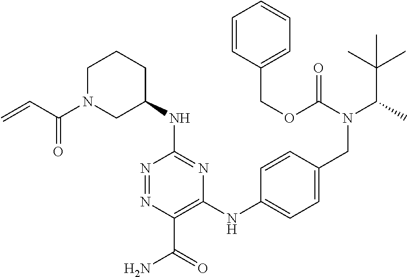 [SMILES: C=CC(=O)N1CCC[C@@H](Nc2nnc(C(N)=O)c(Nc3ccc(CN(C(=O)OCc4ccccc4)[C@@H](C)C(C)(C)C)cc3)n2)C1] | 615.6 | benzyl 4-(3-((R)-1- acryloylpiperidin-3- ylamino)-6-carbamoyl- 1,2,4-triazin-5- ylamino)benzyl((S)- 3,3-dimethylbutan-2- yl)carbamate | Similar to Example E-16 |
| E-57 | 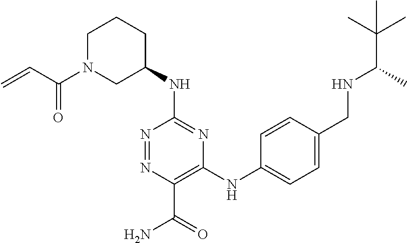 [SMILES: C=CC(=O)N1CCC[C@@H](Nc2nnc(C(N)=O)c(Nc3ccc(CN[C@@H](C)C(C)(C)C)cc3)n2)C1] | 481.3 | 3-((R)-1- acryloylpiperidin-3- ylamino)-5-(4-(((S)- 3,3-dimethylbutan-2- ylamino)methyl)phenyl- amino)-1,2,4-triazine- 6-carboxamide | Similar to Example E-16 |
| E-58 | 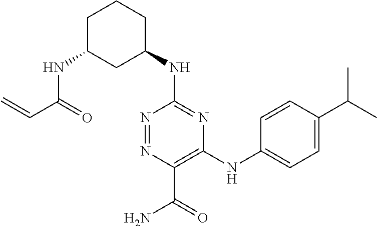 [SMILES: C=CC(=O)N[C@@H]1CCC[C@@H](Nc2nnc(C(N)=O)c(Nc3ccc(C(C)C)cc3)n2)C1] | 424.4 | 3-((1R,3R)-3- acrylamidocyclohexyl- amino)-5-(4- isopropylphenylamino)- 1,2,4-triazine-6- carboxamide | Similar to Example E-16 |
| E-59 | 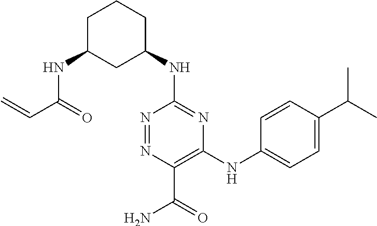 [SMILES: C=CC(=O)N[C@H]1CCC[C@@H](Nc2nnc(C(N)=O)c(Nc3ccc(C(C)C)cc3)n2)C1] | 424.3 | 3-((1R,3S)-3- acrylamidocyclohexyl- amino)-5-(4- isopropylphenylamino)- 1,2,4-triazine-6- carboxamide | Similar to Example E-16 |
| E-60 | 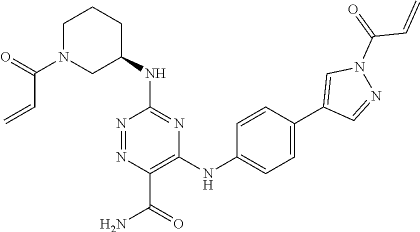 [SMILES: C=CC(=O)N1CCC[C@@H](Nc2nnc(C(N)=O)c(Nc3ccc(-c4cnn(C(=O)C=C)c4)cc3)n2)C1] | 488.2 | (R)-5-(4-(1-acryloyl- 1H-pyrazol-4- yl)phenylamino)-3-(1- acryloylpiperidin-3- ylamino)-1,2,4-triazine- 6-carboxamide | Similar to Example E-16 |
| E-61 | 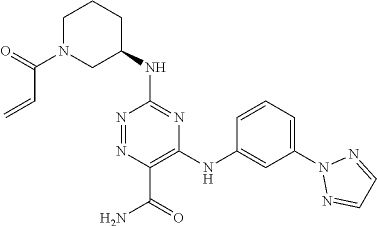 [SMILES: C=CC(=O)N1CCC[C@@H](Nc2nnc(C(N)=O)c(Nc3cccc(-n4nccn4)c3)n2)C1] | 435.3 | (R)-5-(3-(2H-1,2,3- triazol-2- yl)phenylamino)-3-(1- acryloylpiperidin-3- ylamino)-1,2,4-triazine- 6-carboxamide | Similar to Example E-16 |
| E-62 | 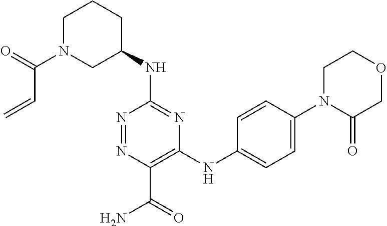 [SMILES: C=CC(=O)N1CCC[C@@H](Nc2nnc(C(N)=O)c(Nc3ccc(N4CCOCC4=O)cc3)n2)C1] | 467.2 | (R)-3-(1- acryloylpiperidin-3- ylamino)-5-(4-(3- oxomorpholino)phenyl- amino)-1,2,4-triazine- 6-carboxamide | Similar to Example E-16 |
| E-63 | 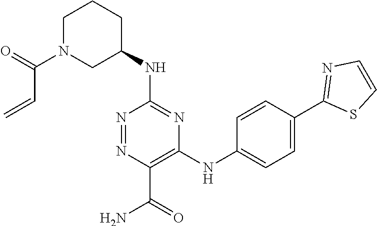 [SMILES: C=CC(=O)N1CCC[C@@H](Nc2nnc(C(N)=O)c(Nc3ccc(-c4nccs4)cc3)n2)C1] | 451.2 | (R)-3-(1- acryloylpiperidin-3- ylamino)-5-(4-(thiazol- 2-yl)phenylamino)- 1,2,4-triazine-6- carboxamide | Similar to Example E-16 |
| E-64 | 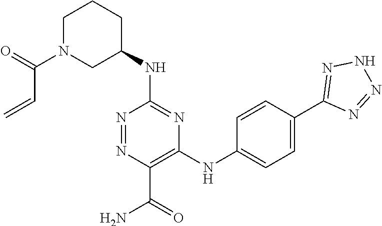 [SMILES: C=CC(=O)N1CCC[C@@H](Nc2nnc(C(N)=O)c(Nc3ccc(-c4nn[nH]n4)cc3)n2)C1] | 436.1 | (R)-5-(4-(2H-tetrazol- 5-yl)phenylamino)-3- (1-acryloylpiperidin-3- ylamino)-1,2,4-triazine- 6-carboxamide | Similar to Example E-16 |
| E-65 | 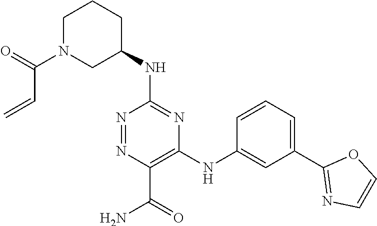 [SMILES: C=CC(=O)N1CCC[C@@H](Nc2nnc(C(N)=O)c(Nc3cccc(-c4ncco4)c3)n2)C1] | 435.3 | (R)-3-(1- acryloylpiperidin-3- ylamino)-5-(3-(oxazol- 2-yl)phenylamino)- 1,2,4-triazine-6- carboxamide | Similar to Example E-16 |
| E-66 | 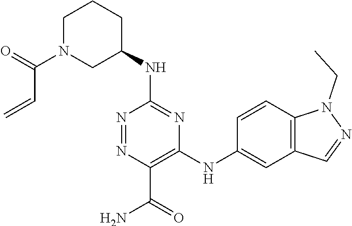 [SMILES: C=CC(=O)N1CCC[C@@H](Nc2nnc(C(N)=O)c(Nc3ccc4c(cnn4CC)c3)n2)C1] | 436.3 | (R)-3-(1- acryloylpiperidin-3- ylamino)-5-(1-ethyl- 1H-indazol-5-ylamino)- 1,2,4-triazine-6- carboxamide | Similar to Example E-16 |
| E-67 | 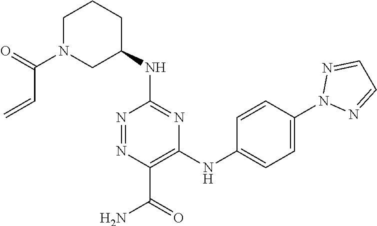 [SMILES: C=CC(=O)N1CCC[C@@H](Nc2nnc(C(N)=O)c(Nc3ccc(-n4nccn4)cc3)n2)C1] | 435.3 | (R)-5-(4-(2H-1,2,3- triazol-2- yl)phenylamino)-3-(1- acryloylpiperidin-3- ylamino)-1,2,4-triazine- 6-carboxamide | Similar to Example E-16 |
| E-68 | 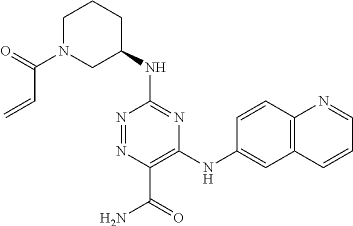 [SMILES: C=CC(=O)N1CCC[C@@H](Nc2nnc(C(N)=O)c(Nc3ccc4ncccc4c3)n2)C1] | 419.1 | (R)-3-(1- acryloylpiperidin-3- ylamino)-5-(quinolin-6- ylamino)-1,2,4-triazine- 6-carboxamide | Similar to Example E-16 |
| E-69 | 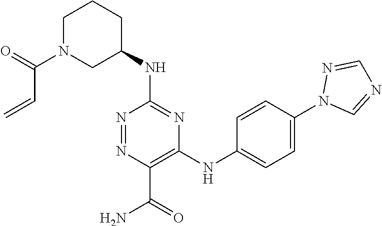 [SMILES: C=CC(=O)N1CCC[C@@H](Nc2nnc(C(N)=O)c(Nc3ccc(-n4cncn4)cc3)n2)C1] | 435.1 | (R)-5-(4-(1H-1,2,4- triazol-1- yl)phenylamino)-3-(1- acryloylpiperidin-3- ylamino)-1,2,4-triazine- 6-carboxamide | Similar to Example E-16 |
| E-70 | 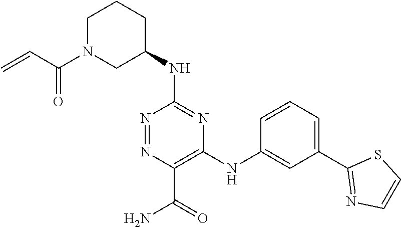 [SMILES: C=CC(=O)N1CCC[C@@H](Nc2nnc(C(N)=O)c(Nc3cccc(-c4nccs4)c3)n2)C1] | 451.3 | (R)-3-(1- acryloylpiperidin-3- ylamino)-5-(3-(thiazol- 2-yl)phenylamino)- 1,2,4-triazine-6- carboxamide | Similar to Example E-16 |
| E-71 | 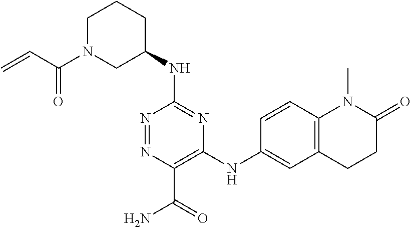 [SMILES: C=CC(=O)N1CCC[C@@H](Nc2nnc(C(N)=O)c(Nc3ccc4c(c3)CCC(=O)N4C)n2)C1] | 451.2 | (R)-3-(1- acryloylpiperidin-3- ylamino)-5-(1-methyl- 2-oxo-1,2,3,4- tetrahydroquinolin-6- ylamino)-1,2,4-triazine- 6-carboxamide | Similar to Example E-16 |
| E-72 | 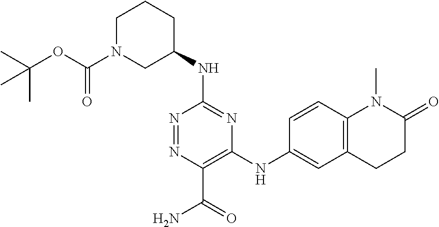 [SMILES: CN1C(=O)CCc2cc(Nc3nc(N[C@@H]4CCCN(C(=O)OC(C)(C)C)C4)nnc3C(N)=O)ccc21] | 497.4 | (R)-tert-butyl 3-(6- carbamoyl-5-(1- methyl-2-oxo-1,2,3,4- tetrahydroquinolin-6- ylamino)-1,2,4-triazin- 3-ylamino)piperidine- 1-carboxylate | Similar to Example E-16 |
| E-73 | 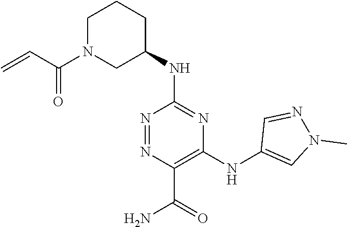 [SMILES: C=CC(=O)N1CCC[C@@H](Nc2nnc(C(N)=O)c(Nc3cnn(C)c3)n2)C1] | 372.2 | (R)-3-(1- acryloylpiperidin-3- ylamino)-5-(1-methyl- 1H-pyrazol-4- ylamino)-1,2,4-triazine- 6-carboxamide | Similar to Example E-16 |
| E-74 | 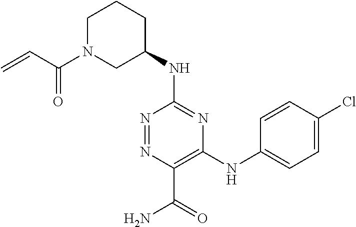 [SMILES: C=CC(=O)N1CCC[C@@H](Nc2nnc(C(N)=O)c(Nc3ccc(Cl)cc3)n2)C1] | 402.1 | (R)-3-(1- acryloylpiperidin-3- ylamino)-5-(4- chlorophenylamino)- 1,2,4-triazine-6- carboxamide | Similar to Example E-16 |
| E-75 | 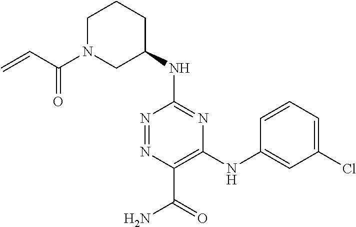 [SMILES: C=CC(=O)N1CCC[C@@H](Nc2nnc(C(N)=O)c(Nc3cccc(Cl)c3)n2)C1] | 402.1 | (R)-3-(1- acryloylpiperidin-3- ylamino)-5-(3- chlorophenylamino)- 1,2,4-triazine-6- carboxamide | Similar to Example E-16 |
| E-76 | 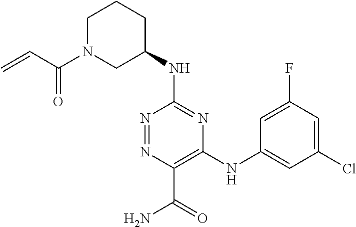 [SMILES: C=CC(=O)N1CCC[C@@H](Nc2nnc(C(N)=O)c(Nc3cc(F)cc(Cl)c3)n2)C1] | 420.1 | (R)-3-(1- acryloylpiperidin-3- ylamino)-5-(3-chloro- 5-fluorophenylamino)- 1,2,4-triazine-6- carboxamide | Similar to Example E-16 |
| E-77 | 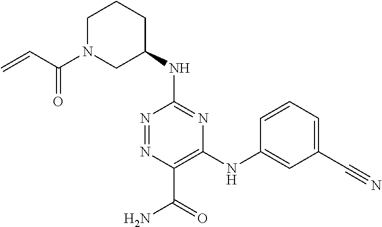 [SMILES: C=CC(=O)N1CCC[C@@H](Nc2nnc(C(N)=O)c(Nc3cccc(C#N)c3)n2)C1] | 393.2 | (R)-3-(1- acryloylpiperidin-3- ylamino)-5-(3- cyanophenylamino)- 1,2,4-triazine-6- carboxamide | Similar to Example E-16 |
| E-78 | 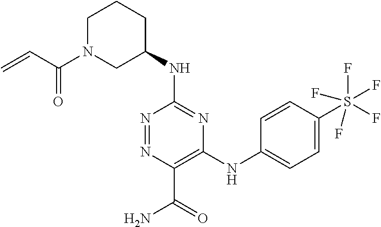 [SMILES: C=CC(=O)N1CCC[C@@H](Nc2nnc(C(N)=O)c(Nc3ccc(S(F)(F)(F)(F)F)cc3)n2)C1] | 494.3 | (R)-3-(1- acryloylpiperidin-3- ylamino)-5-(4-(penta- fluorothio)phenyl- amino)-1,2,4-triazine- 6-carboxamide | Similar to Example E-16 |
| E-79 | 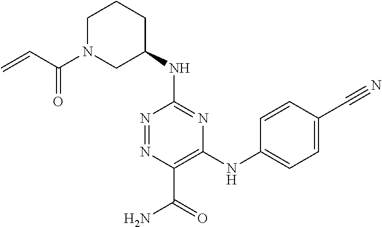 [SMILES: C=CC(=O)N1CCC[C@@H](Nc2nnc(C(N)=O)c(Nc3ccc(C#N)cc3)n2)C1] | 393.1 | (R)-3-(1- acryloylpiperidin-3- ylamino)-5-(4- cyanophenylamino)- 1,2,4-triazine-6- carboxamide | Similar to Example E-16 |
| E-80 | 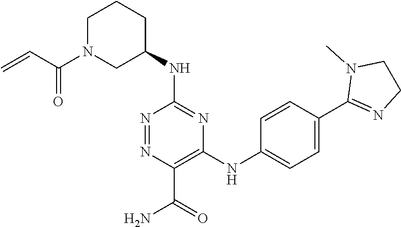 [SMILES: C=CC(=O)N1CCC[C@@H](Nc2nnc(C(N)=O)c(Nc3ccc(C4=NCCN4C)cc3)n2)C1] | 450.2 | (R)-3-(1- acryloylpiperidin-3- ylamino)-5-(4-(1- methyl-4,5-dihydro- 1H-imidazol-2- yl)phenylamino)-1,2,4- triazine-6-carboxamide | Similar to Example E-16 |
| E-81 | 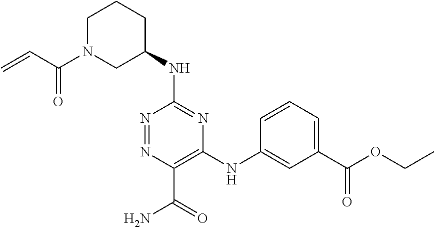 [SMILES: C=CC(=O)N1CCC[C@@H](Nc2nnc(C(N)=O)c(Nc3cccc(C(=O)OCC)c3)n2)C1] | 440.1 | (R)-ethyl 3-(3-(1- acryloylpiperidin-3- ylamino)-6-carbamoyl- 1,2,4-triazin-5- ylamino)benzoate | Similar to Example E-16 |
| E-82 | 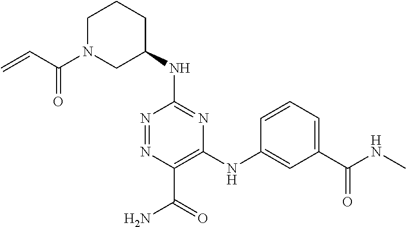 [SMILES: C=CC(=O)N1CCC[C@@H](Nc2nnc(C(N)=O)c(Nc3cccc(C(=O)NC)c3)n2)C1] | 425.1 | (R)-3-(1- acryloylpiperidin-3- ylamino)-5-(3- (methylcarbamoyl) phenylamino)-1,2,4- triazine-6-carboxamide | Similar to Example E-16 |
| E-83 | 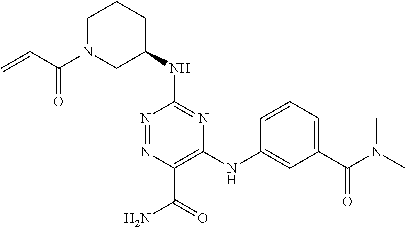 [SMILES: C=CC(=O)N1CCC[C@@H](Nc2nnc(C(N)=O)c(Nc3cccc(C(=O)N(C)C)c3)n2)C1] | 429.1 | (R)-3-(1- acryloylpiperidin-3- ylamino)-5-(3- (dimethylcarbamoyl) phenylamino)-1,2,4- triazine-6-carboxamide | Similar to Example E-16 |
| E-84 | 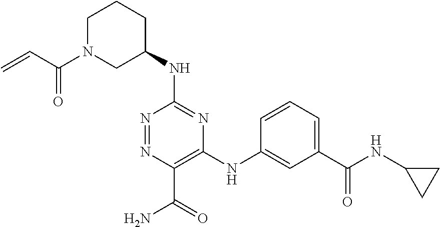 [SMILES: C=CC(=O)N1CCC[C@@H](Nc2nnc(C(N)=O)c(Nc3cccc(C(=O)NC4CC4)c3)n2)C1] | 451.1 | (R)-3-(1- acryloylpiperidin-3- ylamino)-5-(3- (cyclopropyl- carbamoyl)phenyl- amino)-1,2,4- triazine-6-carboxamide | Similar to Example E-16 |
| E-85 | 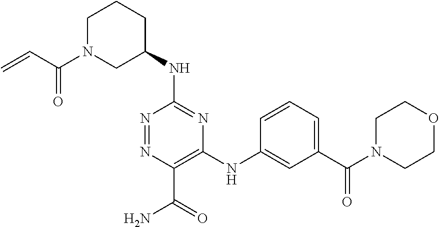 [SMILES: C=CC(=O)N1CCC[C@@H](Nc2nnc(C(N)=O)c(Nc3cccc(C(=O)N4CCOCC4)c3)n2)C1] | 481.1 | (R)-3-(1- acryloylpiperidin-3- ylamino)-5-(3- (morpholine-4- carbonyl)phenylamino)- 1,2,4-triazine-6- carboxamide | Similar to Example E-16 |
| E-86 | 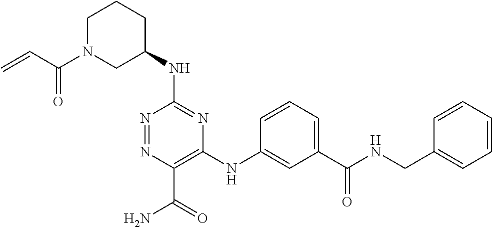 [SMILES: C=CC(=O)N1CCC[C@@H](Nc2nnc(C(N)=O)c(Nc3cccc(C(=O)NCc4ccccc4)c3)n2)C1] | 501.1 | (R)-3-(1- acryloylpiperidin-3- ylamino)-5-(3-(benzyl- carbamoyl)phenyl- amino)-1,2,4-triazine- 6-carboxamide | Similar to Example E-16 |
| E-87 | 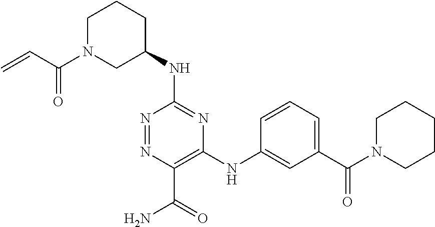 [SMILES: C=CC(=O)N1CCC[C@@H](Nc2nnc(C(N)=O)c(Nc3cccc(C(=O)N4CCCCC4)c3)n2)C1] | 479.2 | (R)-3-(1- acryloylpiperidin-3- ylamino)-5-(3- (piperidine-1- carbonyl)phenylamino)- 1,2,4-triazine-6- carboxamide | Similar to Example E-16 |
| E-88 | 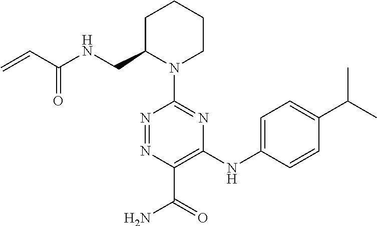 [SMILES: C=CC(=O)NC[C@H]1CCCCN1c1nnc(C(N)=O)c(Nc2ccc(C(C)C)cc2)n1] | 424.1 | (R)-3-(2-(acrylamido- methyl)piperidin-1-yl)- 5-(4-isopropyl- phenylamino)-1,2,4- triazine-6-carboxamide | Similar to Example E-16 |
| E-89 | 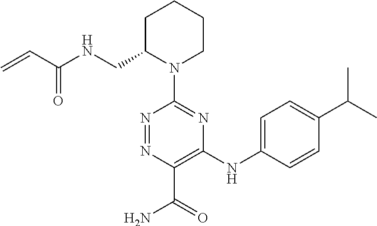 [SMILES: C=CC(=O)NC[C@@H]1CCCCN1c1nnc(C(N)=O)c(Nc2ccc(C(C)C)cc2)n1] | 424.1 | (S)-3-(2-(acrylamido- methyl)piperidin-1-yl)- 5-(4-isopropyl- phenylamino)-1,2,4- triazine-6-carboxamide | Similar to Example E-16 |
| E-90 | 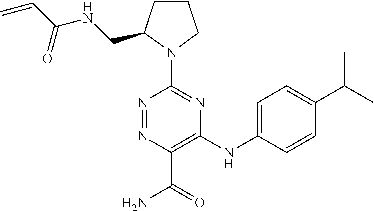 [SMILES: C=CC(=O)NC[C@H]1CCCN1c1nnc(C(N)=O)c(Nc2ccc(C(C)C)cc2)n1] | 410.1 | (R)-3-(2-(acrylamido- methyl)pyrrolidin-1-yl)- 5-(4-isopropylphenyl- amino)-1,2,4-triazine- 6-carboxamide | Similar to Example E-16 |
| E-91 | 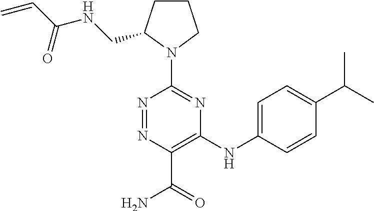 [SMILES: C=CC(=O)NC[C@@H]1CCCN1c1nnc(C(N)=O)c(Nc2ccc(C(C)C)cc2)n1] | 410.1 | (S)-3-(2-(acrylamido- methyl)pyrrolidin-1-yl)- 5-(4-isopropylphenyl- amino)-1,2,4-triazine- 6-carboxamide | Similar to Example E-16 |
| E-92 | 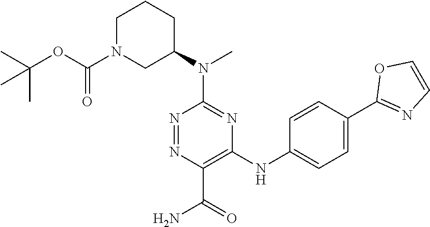 [SMILES: CN(c1nnc(C(N)=O)c(Nc2ccc(-c3ncco3)cc2)n1)[C@@H]1CCCN(C(=O)OC(C)(C)C)C1] | 495.3 | tert-butyl (R)-3-((6- carbamoyl-5-((4- (oxazol-2- yl)phenyl)amino)- 1,2,4-triazin-3- yl)(methyl)amino) piperidine-1- carboxylate | Similar to Example E-16 |

### Example E-93. 5-((2R,3R)-3-acrylamido-2-methylpiperidin-1-yl)-3-(4-(1-cyclopropylpiperidin-4-yl)phenylamino)pyrazine-2-carboxamide

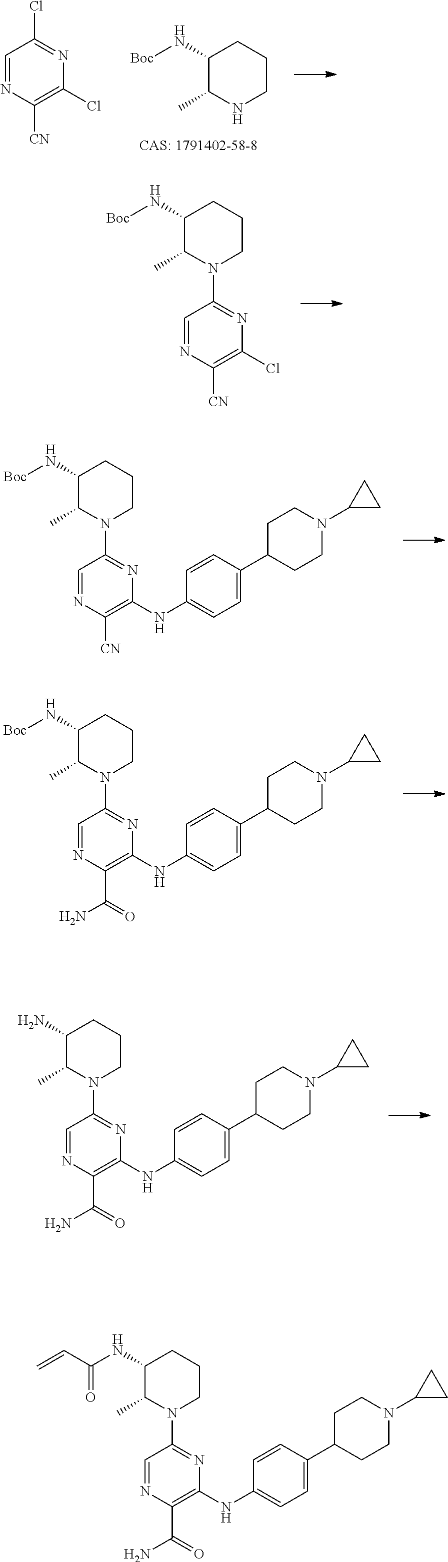

The mixture of 3,5-dichloropyrazine-2-carbonitrile (680 mg, 3.91 mmol), tert-butyl ((2R,3R)-2-methylpiperidin-3-yl)carbamate (CAS: 1791402-58-8; 920 mg, 4.3 mmol) and DIEA (1.02 mL, 5.87 mmol) in 15 mL dry DMF was stirred at RT for overnight. It was concentrated in vacuo and subjected to flash column using 0 to 25% EtOAc in DCM to isolate tert-butyl ((2R,3R)-1-(6-chloro-5-cyanopyrazin-2-yl)-2-methylpiperidin-3-yl)carbamate (1190 mg, 86%). This compound (120 mg, 0.34 mmol) was mixed with 4-(1-cyclopropylpiperidin-4-yl)aniline (145 mg, 0.68 mmol), Pd(OAc)₂ (22 mg, 0.1 mmol), BINAP (62 mg, 0.1 mmol), powder cesium carbonate (440 mg, 1.36 mmol) in 20 mL dioxane. It was degassed with nitrogen stream for 5 min and stirred at 115° C. under nitrogen atmosphere for 1 hour. It was cooled to RT, diluted with 100 mL EtOAc, filtered through a ChemGlass OP-6602-12 filter, concentrated in vacuo and subjected to flash column using 0 to 90% EtOAc in DCM to isolate tert-butyl ((2R,3R)-1-(5-cyano-6-((4-(1-cyclopropylpiperidin-4-yl)phenyl)amino)pyrazin-2-yl)-2-methylpiperidin-3-yl)carbamate. It was dissolved in 10 mL methanol. To it were added 300 μL triethylamine, 1 mL DMSO, 1 NaOH pellet and then 0.5 mL 30% H₂O₂. The mixture was stirred at RT for 1 hour, diluted with 5 mL acetonitrile, stirred, concentrated, diluted with 100 mL EtOAc, washed with water, concentrated in vacuo to dryness to afford crude tert-butyl ((2R,3R)-1-(5-carbamoyl-6-((4-(1-cyclopropylpiperidin-4-yl)phenyl)amino)pyrazin-2-yl)-2-methylpiperidin-3-yl)carbamate. It was stirred in 3 mL methanol and 9 mL “4N HCl in dioxane” at RT for 30 min and concentrated in vacuo to dryness to give crude 5-((2R,3R)-3-amino-2-methylpiperidin-1-yl)-3-((4-(1-cyclopropylpiperidin-4-yl)phenyl)amino)pyrazine-2-carboxamide HCl salt. It was dissolved in 4 mL DMF and stirred in ice bath. To it were added DIEA (350 μL, 2.04 mmol) and then acryloyl chloride (28 μL, 0.34 mmol). The mixture was stirred for 15 min and quenched with 0.5 mL TFA. It was directly subjected to reverse phase preparative HPLC to isolate the title compound, 5-((2R,3R)-3-acrylamido-2-methylpiperidin-1-yl)-3-(4-(1-cyclopropylpiperidin-4-yl)phenylamino)pyrazine-2-carboxamide (72 mg). MS found for C₂₈H₃₇N₇O₂ as (M+H)⁺ 504.4 and (M−H)⁻ 502.2.

### Example E-94. (S)-5-(1-acryloylpiperidin-3-ylamino)-3-(4-(pyrimidin-2-yl)phenylamino)pyrazine-2-carboxamide

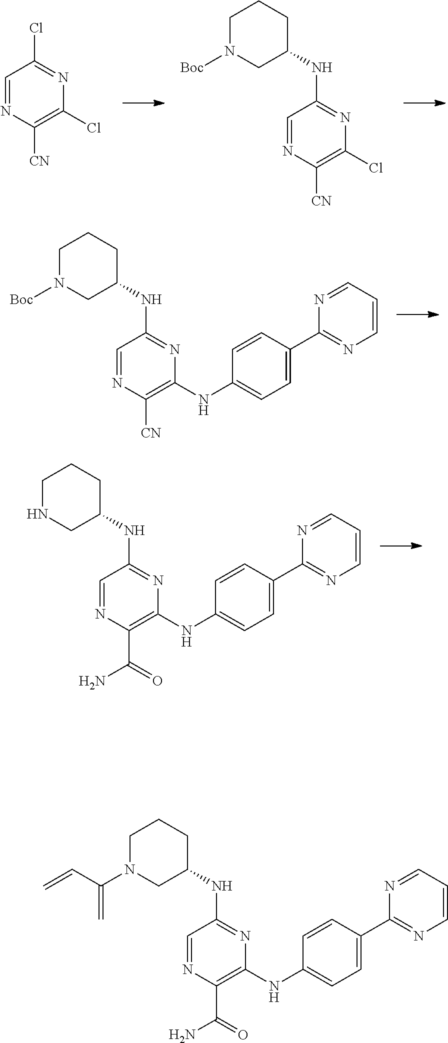 [SMILES: N#Cc1ncc(Cl)nc1Cl ; CC(C)(C)OC(=O)N1CCC[C@H](Nc2cnc(C#N)c(Cl)n2)C1 ; CC(C)(C)OC(=O)N1CCC[C@H](Nc2cnc(C#N)c(Nc3ccc(-c4ncccn4)cc3)n2)C1 ; NC(=O)c1ncc(N[C@H]2CCCNC2)nc1Nc1ccc(-c2ncccn2)cc1 ; C=CC(=C)N1CCC[C@H](Nc2cnc(C(N)=O)c(Nc3ccc(-c4ncccn4)cc3)n2)C1]

3,5-Dichloropyrazine-2-carbonitrile (1600 mg, 9.18 mmol) was dissolved in 50 mL dry acetonitrile. To it were added tert-butyl (S)-3-aminopiperidine-1-carboxylate (2020 mg, 10.1 mmol) and the DIEA (2.40 mL, 13.7 mmol) dropwise. The mixture was stirred at RT for 30m, concentrated in vacuo, diluted with EtOAc, washed with water three times, and subjected to flash column using 5 to 20% EtOAc in DCM to isolate tert-butyl (S)-3-((6-chloro-5-cyanopyrazin-2-yl)amino)piperidine-1-carboxylate (3000 mg, 96%). This compound (120 mg, 0.35 mmol) was mixed with 4-(pyrimidin-2-yl)aniline (120 mg, 0.70 mmol), Pd(OAc)₂ (25 mg, 0.11 mmol), BINAP (68 mg, 0.11 mmol), powder cesium carbonate (570 mg, 1.75 mmol) in 20 mL dioxane. It was degassed with nitrogen stream for 5 min and stirred at 115° C. under nitrogen atmosphere for 1 hour. It was cooled to RT, diluted with 100 mL EtOAc, filtered through a ChemGlass OP-6602-12 filter, concentrated in vacuo and subjected to flash column using 0 to 5% MeOH in DCM to isolate tert-butyl (S)-3-((5-cyano-6-((4-(pyrimidin-2-yl)phenyl)amino)pyrazin-2-yl)amino)piperidine-1-carboxylate. It was dissolved in 6 mL TFA. To it was added 1 mL concentrated H₂SO₄, and the mixture was stirred in 80° C. bath for 30 min. It was cooled to RT, diluted with 5 mL water and subjected to reverse preparative HPLC to isolate (S)-5-(piperidin-3-ylamino)-3-((4-(pyrimidin-2-yl)phenyl)amino)pyrazine-2-carboxamide HCl salt (170 mg). This compound (38 mg, 0.089 mmol) was dissolved in 2 mL NMP and stirred in ice bath. To it were added DIEA (77 μL, 0.445 mmol) and then acryloyl chloride (10 μL, 0.13 mmol). The mixture was stirred for 5 min and quenched with 0.2 mL TFA. It was directly subjected to reverse phase preparative HPLC to isolate the title compound, (S)-5-(1-acryloylpiperidin-3-ylamino)-3-(4-(pyrimidin-2-yl)phenylamino)pyrazine-2-carboxamide (25 mg). MS found for C23H24N8O2 as (M+H)⁺ 445.2 and (M−H)⁻ 443.1.

Using synthetic schemes similar to what shown above for Example E-93 and Example 94, the following compounds have been prepared:

TABLE 9
Additional Compounds of Formula (I)

| | | LC- | | |
|---|---|---|---|---|
| | | MS | | |
| Cmpd | | (ESI): | | |
| # | Structure | m/z | Name | Procedure |
| E-95 | 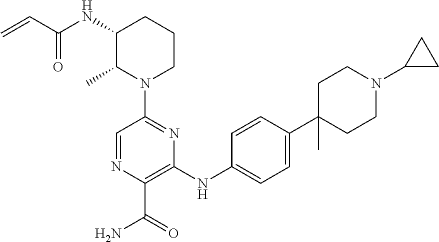 [SMILES: C=CC(=O)N[C@@H]1CCCN(c2cnc(C(N)=O)c(Nc3ccc(C4(C)CCN(C5CC5)CC4)cc3)n2)[C@@H]1C] | 518.2 | 5-((2R,3R)-3- acrylamido-2- methylpiperidin-1-yl)- 3-((4-(1-cyclopropyl-4- methylpiperidin-4- yl)phenyl)amino)pyrazine- 2-carboxamide | Similar to Example E-93 |
| E-96 | 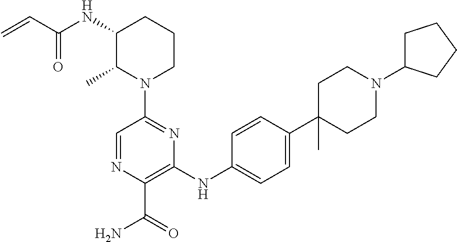 [SMILES: C=CC(=O)N[C@@H]1CCCN(c2cnc(C(N)=O)c(Nc3ccc(C4(C)CCN(C5CCCC5)CC4)cc3)n2)[C@@H]1C] | 546.3 | 5-((2R,3R)-3- acrylamido-2- methylpiperidin-1-yl)- 3-((4-(1-cyclopentyl-4- methylpiperidin-4- yl)phenyl)amino)pyrazine- 2-carboxamide | Similar to Example E-93 |
| E-97 | 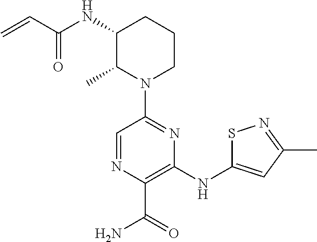 [SMILES: C=CC(=O)N[C@@H]1CCCN(c2cnc(C(N)=O)c(Nc3cc(C)ns3)n2)[C@@H]1C] | 402.3 | 5-((2R,3R)-3- acrylamido-2- methylpiperidin-1-yl)- 3-(3-methylisothiazol- 5-ylamino)pyrazine-2- carboxamide | Similar to Example E-93 |
| E-98 | 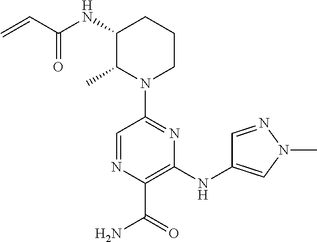 [SMILES: C=CC(=O)N[C@@H]1CCCN(c2cnc(C(N)=O)c(Nc3cnn(C)c3)n2)[C@@H]1C] | 385.3 | 5-((2R,3R)-3- acrylamido-2- methylpiperidin-1-yl)- 3-(1-methyl-1H- pyrazol-4- ylamino)pyrazine-2- carboxamide | Similar to Example E-93 |
| E-99 | 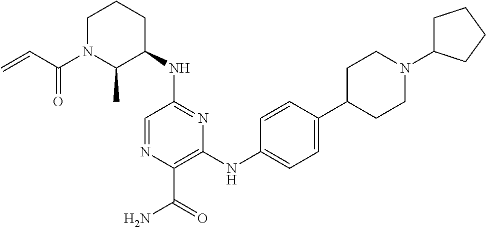 [SMILES: C=CC(=O)N1CCC[C@@H](Nc2cnc(C(N)=O)c(Nc3ccc(C4CCN(C5CCCC5)CC4)cc3)n2)[C@H]1C] | 532.6 | 5-((2R,3R)-1-acryloyl- 2-methylpiperidin-3- ylamino)-3-(4-(1- cyclopentylpiperidin-4- yl)phenylamino)pyrazine- 2-carboxamide | Similar to Example E-93 |
| E-100 | 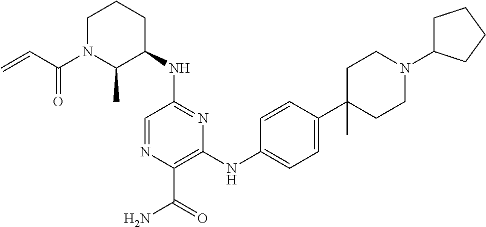 [SMILES: C=CC(=O)N1CCC[C@@H](Nc2cnc(C(N)=O)c(Nc3ccc(C4(C)CCN(C5CCCC5)CC4)cc3)n2)[C@H]1C] | 546.7 | 5-((2R,3R)-1-acryloyl- 2-methylpiperidin-3- ylamino)-3-(4-(1- cyclopentyl-4- methylpiperidin-4- yl)phenylamino)pyrazine- 2-carboxamide | Similar to Example E-93 |
| E-101 | 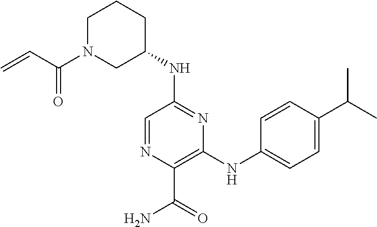 [SMILES: C=CC(=O)N1CCC[C@H](Nc2cnc(C(N)=O)c(Nc3ccc(C(C)C)cc3)n2)C1] | 409.1 | (S)-5-(1- acryloylpiperidin-3- ylamino)-3-(4- isopropylphenylamino) pyrazine-2- carboxamide | Similar to Example E-93 |
| E-102 | 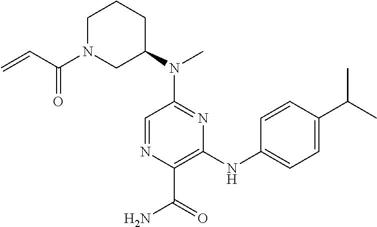 [SMILES: C=CC(=O)N1CCC[C@@H](N(C)c2cnc(C(N)=O)c(Nc3ccc(C(C)C)cc3)n2)C1] | 423.2 | (R)-5-((1- acryloylpiperidin-3- yl)(methyl)amino)-3-(4- isopropylphenylamino) pyrazine-2- carboxamide | Similar to Example E-93 |
| E-103 | 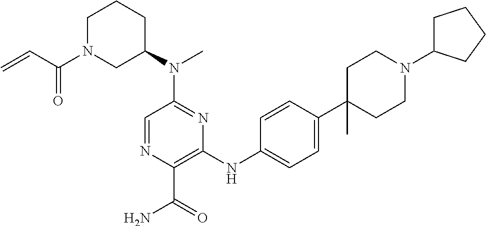 [SMILES: C=CC(=O)N1CCC[C@@H](N(C)c2cnc(C(N)=O)c(Nc3ccc(C4(C)CCN(C5CCCC5)CC4)cc3)n2)C1] | 546.7 | (R)-5-((1- acryloylpiperidin-3- yl)(methyl)amino)-3-(4- (1-cyclopentyl-4- methylpiperidin-4- yl)phenylamino)pyrazine- 2-carboxamide | Similar to Example E-93 |
| E-104 | 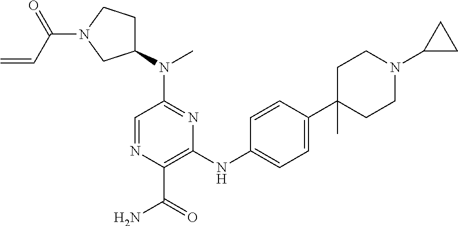 [SMILES: C=CC(=O)N1CC[C@@H](N(C)c2cnc(C(N)=O)c(Nc3ccc(C4(C)CCN(C5CC5)CC4)cc3)n2)C1] | 504.5 | (R)-5-((1- acryloylpyrrolidin-3- yl)(methyl)amino)-3-(4- (1-cyclopropyl-4- methylpiperidin-4- yl)phenylammo)pyrazine- 2-carboxamide | Similar to Example E-93 |
| E-105 | 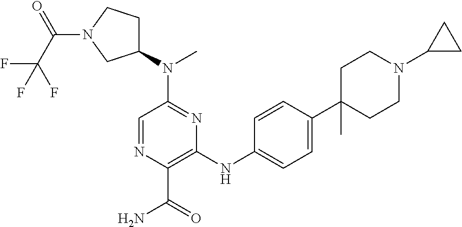 [SMILES: CN(c1cnc(C(N)=O)c(Nc2ccc(C3(C)CCN(C4CC4)CC3)cc2)n1)[C@@H]1CCN(C(=O)C(F)(F)F)C1] | 546.5 | (R)-3-(4-(1- cyclopropyl-4- methylpiperidin-4- yl)phenylamino)-5- (methyl(1-(2,2,2- trifluoroacetyl)pyrrolidin- 3-yl)amino)pyrazine- 2-carboxamide | Similar to Example E-93 |
| E-106 | 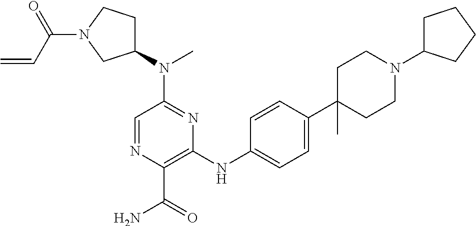 [SMILES: C=CC(=O)N1CC[C@@H](N(C)c2cnc(C(N)=O)c(Nc3ccc(C4(C)CCN(C5CCCC5)CC4)cc3)n2)C1] | 532.7 | (R)-5-((1- acryloylpyrrolidin-3- yl)(methyl)amino)-3-(4- (1-cyclopentyl-4- methylpiperidin-4- yl)phenylamino)pyrazine- 2-carboxamide | Similar to Example E-93 |
| E-107 | 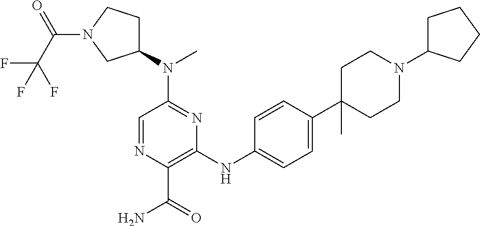 [SMILES: CN(c1cnc(C(N)=O)c(Nc2ccc(C3(C)CCN(C4CCCC4)CC3)cc2)n1)[C@@H]1CCN(C(=O)C(F)(F)F)C1] | 574.5 | (R)-3-(4-(1- cyclopentyl-4- methylpiperidin-4- yl)phenylamino)-5- (methyl(1-(2,2,2- trifluoroacetyl)pyrrolidin- 3-yl)amino)pyrazine- 2-carboxamide | Similar to Example E-93 |
| E-108 | 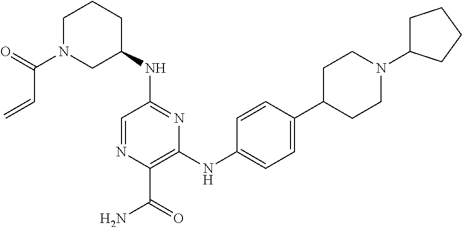 [SMILES: C=CC(=O)N1CCC[C@@H](Nc2cnc(C(N)=O)c(Nc3ccc(C4CCN(C5CCCC5)CC4)cc3)n2)C1] | 518.5 | (R)-5-(1- acryloylpiperidin-3- ylamino)-3-(4-(1- cyclopentylpiperidin-4- yl)phenylamino)pyrazine- 2-carboxamide | Similar to Example E-93 |
| E-109 | 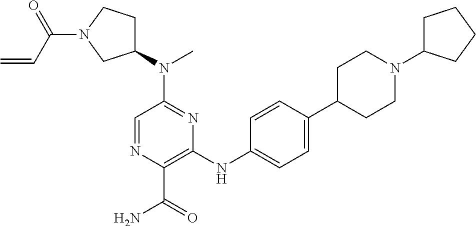 [SMILES: C=CC(=O)N1CC[C@@H](N(C)c2cnc(C(N)=O)c(Nc3ccc(C4CCN(C5CCCC5)CC4)cc3)n2)C1] | 518.6 | (R)-5-((1- acryloylpyrrolidin-3- yl)(methyl)amino)-3-(4- (1- cyclopentylpiperidin-4- yl)phenylamino)pyrazine- 2-carboxamide | Similar to Example E-93 |
| E-110 | 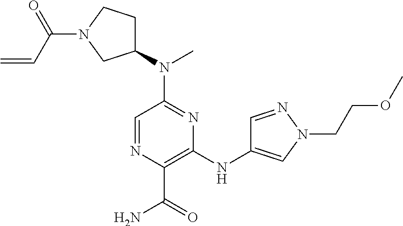 [SMILES: C=CC(=O)N1CC[C@@H](N(C)c2cnc(C(N)=O)c(Nc3cnn(CCOC)c3)n2)C1] | 415.2 | (R)-5-((1- acryloylpyrrolidin-3- yl)(methyl)amino)-3-(1- (2-methoxyethyl)-1H- pyrazol-4- ylamino)pyrazine-2- carboxamide | Similar to Example E-93 |
| E-111 | 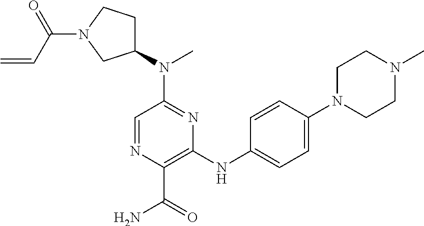 [SMILES: C=CC(=O)N1CC[C@@H](N(C)c2cnc(C(N)=O)c(Nc3ccc(N4CCN(C)CC4)cc3)n2)C1] | 465.3 | (R)-5-((1- acryloylpyrrolidin-3- yl)(methyl)amino)-3-(4- (4-methylpiperazin-1- yl)phenylamino)pyrazine- 2-carboxamide | Similar to Example E-93 |
| E-112 | 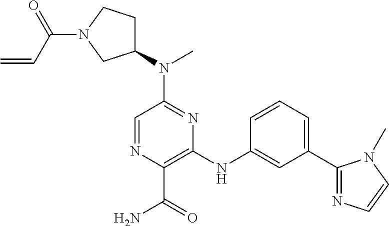 [SMILES: C=CC(=O)N1CC[C@@H](N(C)c2cnc(C(N)=O)c(Nc3cccc(-c4nccn4C)c3)n2)C1] | 447.4 | (R)-5-((1- acryloylpyrrolidin-3- yl)(methyl)amino)-3-(3- (1-methyl-1H-imidazol- 2- yl)phenylamino)pyrazine- 2-carboxamide | Similar to Example E-93 |
| E-113 | 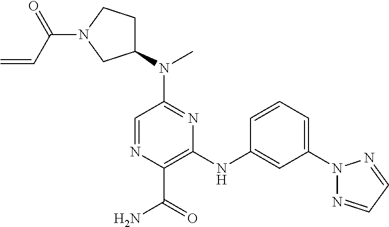 [SMILES: C=CC(=O)N1CC[C@@H](N(C)c2cnc(C(N)=O)c(Nc3cccc(-n4nccn4)c3)n2)C1] | 434.1 | (R)-3-(3-(2H-1,2,3- triazol-2- yl)phenylamino)-5-((1- acryloylpyrrolidin-3- yl)(methyl)amino) pyrazine-2-carboxamide | Similar to Example E-93 |
| E-114 | 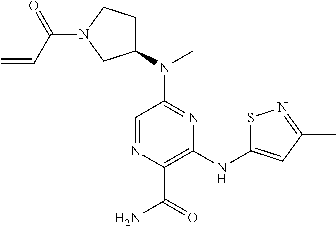 [SMILES: C=CC(=O)N1CC[C@@H](N(C)c2cnc(C(N)=O)c(Nc3cc(C)ns3)n2)C1] | 388.1 | (R)-5-((1- acryloylpyrrolidin-3- yl)(methyl)amino)-3-(3- methylisothiazol-5- ylamino)pyrazine-2- carboxamide | Similar to Example E-93 |
| E-115 | 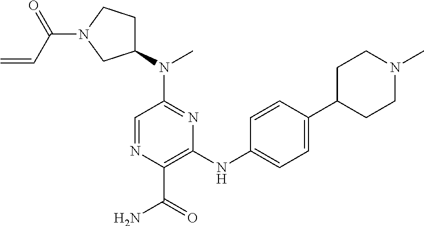 [SMILES: C=CC(=O)N1CC[C@@H](N(C)c2cnc(C(N)=O)c(Nc3ccc(C4CCN(C)CC4)cc3)n2)C1] | 464.2 | (R)-5-((1- acryloylpyrrolidin-3- yl)(methyl)amino)-3-(4- (1-methylpiperidin-4- yl)phenylamino)pyrazine- 2-carboxamide | Similar to Example E-93 |
| E-116 | 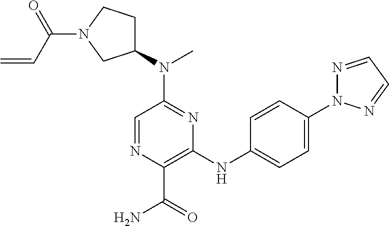 [SMILES: C=CC(=O)N1CC[C@@H](N(C)c2cnc(C(N)=O)c(Nc3ccc(-n4nccn4)cc3)n2)C1] | 434.1 | (R)-3-(4-(2H-1,2,3- triazol-2- yl)phenylamino)-5-((1- acryloylpyrrolidin-3- yl)(methyl)amino) pyrazine-2-carboxamide | Similar to Example E-93 |
| E-117 | 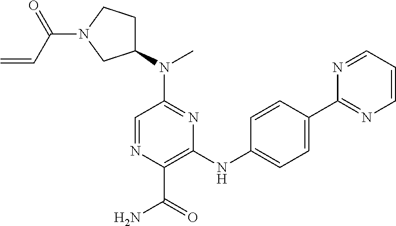 [SMILES: C=CC(=O)N1CC[C@@H](N(C)c2cnc(C(N)=O)c(Nc3ccc(-c4ncccn4)cc3)n2)C1] | 445.2 | (R)-5-((1- acryloylpyrrolidin-3- yl)(methyl)amino)-3-(4- (pyrimidin-2- yl)phenylamino)pyrazine- 2-carboxamide | Similar to Example E-93 |
| E-118 | 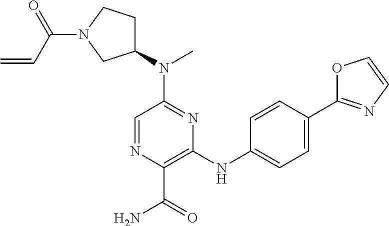 [SMILES: C=CC(=O)N1CC[C@@H](N(C)c2cnc(C(N)=O)c(Nc3ccc(-c4ncco4)cc3)n2)C1] | 434.1 | (R)-5-((1- acryloylpyrrolidin-3- yl)(methyl)amino)-3-(4- (oxazol-2- yl)phenylamino)pyrazine- 2-carboxamide | Similar to Example E-93 |
| E-119 | 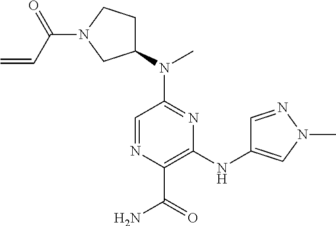 [SMILES: C=CC(=O)N1CC[C@@H](N(C)c2cnc(C(N)=O)c(Nc3cnn(C)c3)n2)C1] | 371.1 | (R)-5-((1- acryloylpyrrolidin-3- yl)(methyl)amino)-3-(1- methyl-1H-pyrazol-4- ylamino)pyrazine-2- carboxamide | Similar to Example E-93 |
| E-120 | 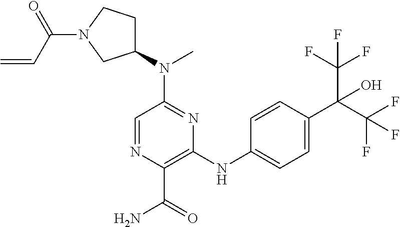 [SMILES: C=CC(=O)N1CC[C@@H](N(C)c2cnc(C(N)=O)c(Nc3ccc(C(O)(C(F)(F)F)C(F)(F)F)cc3)n2)C1] | 533.1 | (R)-5-((1- acryloylpyrrolidin-3- yl)(methyl)amino)-3-(4- (1,1,1,3,3,3-hexafluoro- 2-hydroxypropan-2- yl)phenylamino)pyrazine- 2-carboxamide | Similar to Example E-93 |
| E-121 | 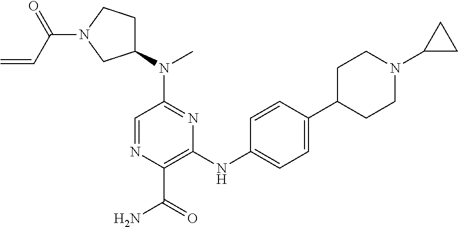 [SMILES: C=CC(=O)N1CC[C@@H](N(C)c2cnc(C(N)=O)c(Nc3ccc(C4CCN(C5CC5)CC4)cc3)n2)C1] | 490.2 | (R)-5-((1- acryloylpyrrolidin-3- yl)(methyl)amino)-3-(4- (1- cyclopropylpiperidin-4- yl)phenylamino)pyrazine- 2-carboxamide | Similar to Example E-93 |
| E-122 | 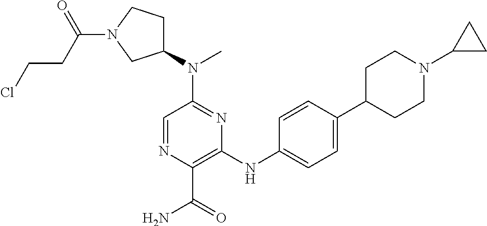 [SMILES: CN(c1cnc(C(N)=O)c(Nc2ccc(C3CCN(C4CC4)CC3)cc2)n1)[C@@H]1CCN(C(=O)CCCl)C1] | 524.2 | (R)-5-((1-(3- chloropropanoyl) pyrrolidin-3- yl)(methyl)amino)-3-(4- (1- cyclopropylpiperidin-4- yl)phenylamino)pyrazine- 2-carboxamide | Similar to Example E-93 |
| E-123 | 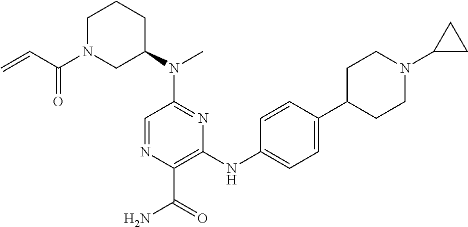 [SMILES: C=CC(=O)N1CCC[C@@H](N(C)c2cnc(C(N)=O)c(Nc3ccc(C4CCN(C5CC5)CC4)cc3)n2)C1] | 504.2 | (R)-5-((1- acryloylpiperidin-3- yl)(methyl)amino)-3-(4- (1- cyclopropylpiperidin-4- yl)phenylamino)pyrazine- 2-carboxamide | Similar to Example E-93 |
| E-124 | 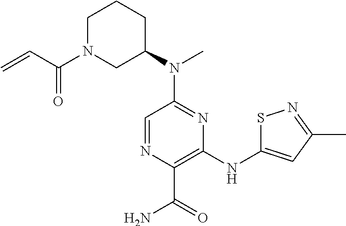 [SMILES: C=CC(=O)N1CCC[C@@H](N(C)c2cnc(C(N)=O)c(Nc3cc(C)ns3)n2)C1] | 402.0 | (R)-5-((1- acryloylpiperidin-3- yl)(methyl)amino)-3-(3- methylisothiazol-5- ylamino)pyrazine-2- carboxamide | Similar to Example E-93 |
| E-125 | 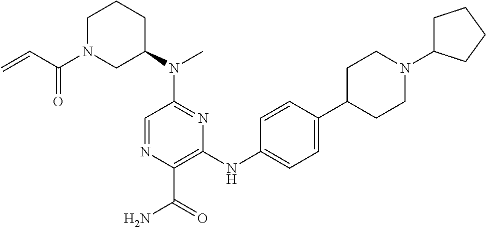 [SMILES: C=CC(=O)N1CCC[C@@H](N(C)c2cnc(C(N)=O)c(Nc3ccc(C4CCN(C5CCCC5)CC4)cc3)n2)C1] | 532.2 | (R)-5-((1- acryloylpiperidin-3- yl)(methyl)amino)-3-(4- (1- cyclopentylpiperidin-4- yl)phenylamino)pyrazine- 2-carboxamide | Similar to Example E-93 |
| E-126 | 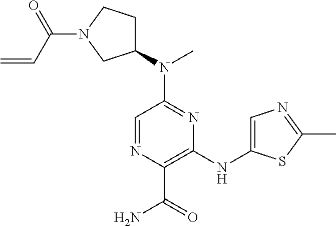 [SMILES: C=CC(=O)N1CC[C@@H](N(C)c2cnc(C(N)=O)c(Nc3cnc(C)s3)n2)C1] | 388.1 | (R)-5-((1- acryloylpyrrolidin-3- yl)(methyl)amino)-3-(2- methylthiazol-5- ylamino)pyrazine-2- carboxamide | Similar to Example E-94 |
| E-127 | 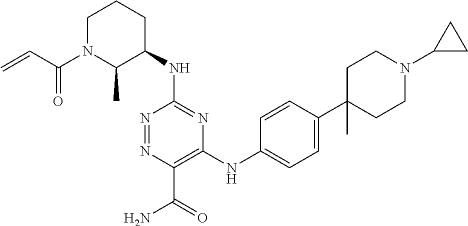 [SMILES: C=CC(=O)N1CCC[C@@H](Nc2nnc(C(N)=O)c(Nc3ccc(C4(C)CCN(C5CC5)CC4)cc3)n2)[C@H]1C] | 519.3 | 3-(((2R,3R)-1-acryloyl- 2-methylpiperidin-3- yl)amino)-5-((4-(1- cyclopropyl-4- methylpiperidin-4- yl)phenyl)amino)-1,2,4- triazine-6-carboxamide | Similar to Example E16 |
| E-128 | 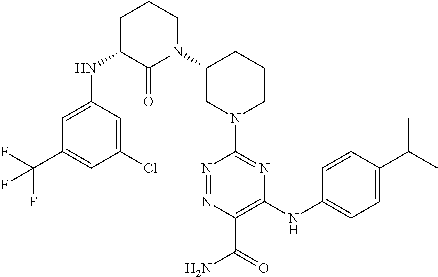 [SMILES: CC(C)c1ccc(Nc2nc(N3CCC[C@@H](N4CCC[C@@H](Nc5cc(Cl)cc(C(F)(F)F)c5)C4=O)C3)nnc2C(N)=O)cc1] | 631.2 | 3-((3R,3′R)-3-((3- chloro-5- (trifluoromethyl)phenyl) amino)-2-oxo-[1,3′- bipiperidin]-1′-yl)-5- ((4- isopropylphenyl)amino)- 1,2,4-triazine-6- carboxamide | Similar to Example E10 |
| E-129 | 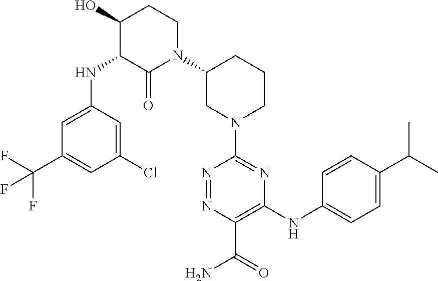 [SMILES: CC(C)c1ccc(Nc2nc(N3CCC[C@@H](N4CC[C@H](O)[C@@H](Nc5cc(Cl)cc(C(F)(F)F)c5)C4=O)C3)nnc2C(N)=O)cc1] | 647.2 | 3-((3R,3′R,4S)-3-((3- chloro-5- (trifluororaethyl)phenyl) amino)-4-hydroxy-2- oxo-[1,3′-bipiperidin]- 1′-yl)-5-((4- isopropylphenyl)amino)- 1,2,4-triazine-6- carboxamide | Similar to Example E10 |
| E-130 | 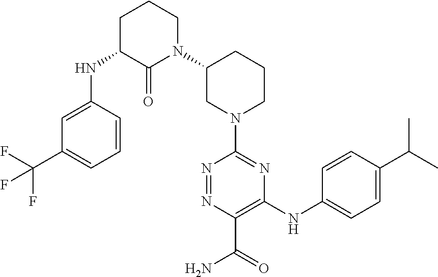 [SMILES: CC(C)c1ccc(Nc2nc(N3CCC[C@@H](N4CCC[C@@H](Nc5cccc(C(F)(F)F)c5)C4=O)C3)nnc2C(N)=O)cc1] | 597.2 | 5-((4- isopropylphenyl)amino)- 3-((3R,3′R)-2-oxo-3- ((3- (trifluoromethyl)phenyl) amino)-[1,3′- bipiperidin]-1′-yl)- 1,2,4-triazine-6- carboxamide | Similar to Example E10 |
| E-131 | 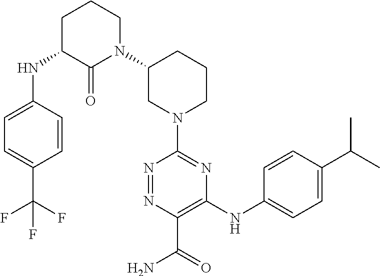 [SMILES: CC(C)c1ccc(Nc2nc(N3CCC[C@@H](N4CCC[C@@H](Nc5ccc(C(F)(F)F)cc5)C4=O)C3)nnc2C(N)=O)cc1] | 597.2 | 5-((4- isopropylphenyl)amino)- 3-((3R,3′R)-2-oxo-3- ((4- (trifluoromethyl)phenyl) amino)-[1,3′- bipiperidin]-1′-yl)- 1,2,4-triazine-6- carboxamide | Similar to Example E10 |
| E-132 | 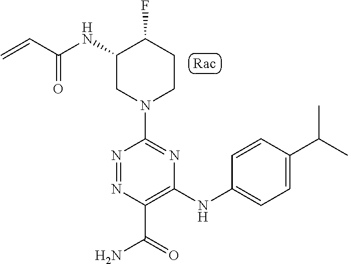 [SMILES: C=CC(=O)N[C@H]1CN(c2nnc(C(N)=O)c(Nc3ccc(C(C)C)cc3)n2)CC[C@H]1F] | 428.2 | rac-3-((3R,4S)-3- acrylamido-4- fluoropiperidin-1-yl)-5- ((4- isopropylphenyl)amino)- 1,2,4-triazine-6- carboxamide | Similar to Example E1 |
| E-133 | 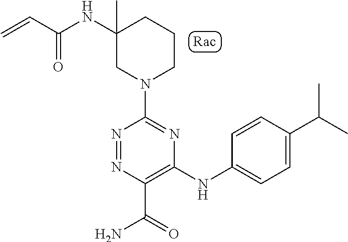 [SMILES: C=CC(=O)NC1(C)CCCN(c2nnc(C(N)=O)c(Nc3ccc(C(C)C)cc3)n2)C1] | 424.2 | 3-(3-acrylamido-3- methylpiperidin-1-yl)- 5-((4- isopropylphenyl)amino)- 1,2,4-triazine-6- carboxamide | Similar to Example E1 |
| E-134 | 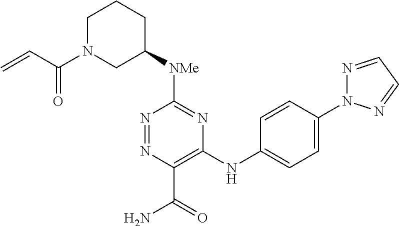 | 449.2 | (R)-5-((4-(2H-1,2,3- triazol-2- yl)phenyl)amino)-3-((1- acryloylpiperidin-3- yl)(methyl)amino)- 1,2,4-triazine-6- carboxamide | Similar to Example E16 |
| E-135 | 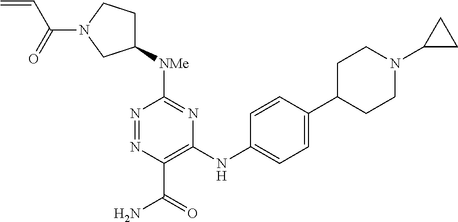 | 491.2 | (R)-3-((1- acryloylpyrrolidin-3- yl)(methyl)amino)-5- ((4-(1- cyclopropylpiperidin-4- yl)phenyl)amino)-1,2,4- triazine-6-carboxamide | Similar to Example E16 |
| E-136 | 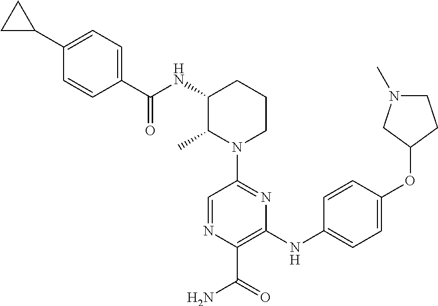 [SMILES: C[C@@H]1[C@H](NC(=O)c2ccc(C3CC3)cc2)CCCN1c1cnc(C(N)=O)c(Nc2ccc(OC3CCN(C)C3)cc2)n1] | 570.4 | 5-((2R,3R)-3-(4- cyclopropylbenzamido)- 2-methylpiperidin-1- yl)-3-((4-((1- methylpyrrolidin-3- yl)oxy)phenyl)amino) pyrazine-2-carboxamide | Similar to Example E93 |
| E-137 | 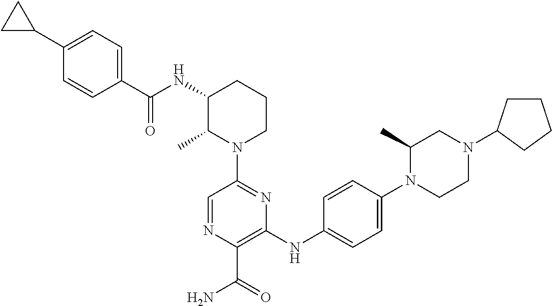 [SMILES: C[C@@H]1[C@H](NC(=O)c2ccc(C3CC3)cc2)CCCN1c1cnc(C(N)=O)c(Nc2ccc(N3CCN(C4CCCC4)C[C@@H]3C)cc2)n1] | 637.6 | 3-((4-((S)-4- cyclopentyl-2- methylpiperazin-1- yl)phenyl)amino)-5- ((2R,3R)-3-(4- cyclopropylbenzamido)- 2-methyipiperidin-1- yl)pyrazine-2- carboxamide | Similar to Example E93 |
| E-138 | 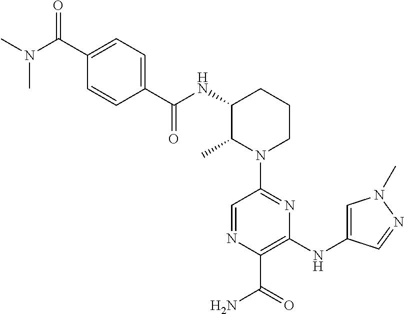 [SMILES: C[C@@H]1[C@H](NC(=O)c2ccc(C(=O)N(C)C)cc2)CCCN1c1cnc(C(N)=O)c(Nc2cnn(C)c2)n1] | 506.4 | N1-((2R,3R)-1-(5- carbamoyl-6-((1- methyl-1H-pyrazol-4- yl)amino)pyrazin-2-yl)- 2-methylpiperidin-3- yl)-N4,N4- dimethylterephthalamide | Similar to Example E93 |
| E-139 | 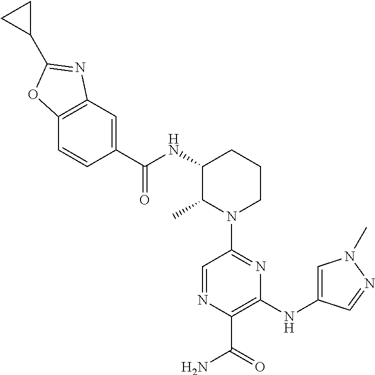 [SMILES: C[C@@H]1[C@H](NC(=O)c2ccc3oc(C4CC4)nc3c2)CCCN1c1cnc(C(N)=O)c(Nc2cnn(C)c2)n1] | 516.4 | N-((2R,3R)-1-(5- carbamoyl-6-((1- methyl-1H-pyrazol-4- yl)amino)pyrazin-2-yl)- 2-methylpiperidin-3- yl)-2- cyclopropylbenzo[d] oxazole-5-carboxamide | Similar to Example E93 |
| E-140 | 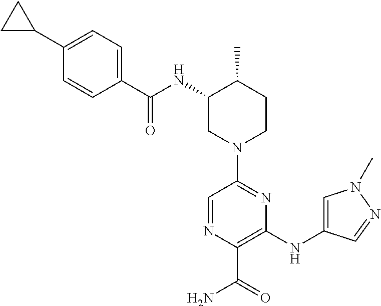 [SMILES: C[C@@H]1CCN(c2cnc(C(N)=O)c(Nc3cnn(C)c3)n2)C[C@@H]1NC(=O)c1ccc(C2CC2)cc1] | 475.4 | 5-((3R,4R)-3-(4- cyclopropylbenzamido)- 4-methylpiperidin-1- yl)-3-((1-methyl-1H- pyrazol-4- yl)amino)pyrazine-2- carboxamide | Similar to Example E93 |
| E-141 | 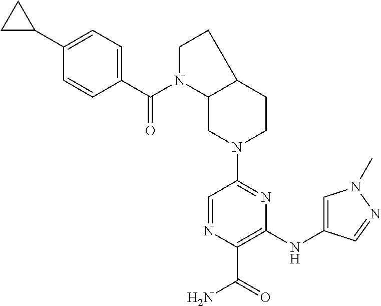 [SMILES: Cn1cc(Nc2nc(N3CCC4CCN(C(=O)c5ccc(C6CC6)cc5)C4C3)cnc2C(N)=O)cn1] | 487.4 | 5-(1-(4- cyclopropylbenzoyl) octahydro-6H-pyrrolo[2,3- c]pyridin-6-yl)-3-((1- methyl-3H-pyrazol-4- yl)amino)pyrazine-2- carboxamide | Similar to Example E93 |
| E-142 | 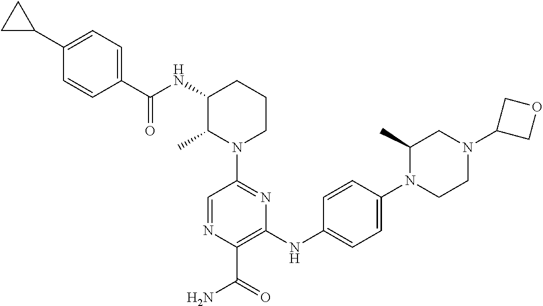 [SMILES: C[C@@H]1[C@H](NC(=O)c2ccc(C3CC3)cc2)CCCN1c1cnc(C(N)=O)c(Nc2ccc(N3CCN(C4COC4)C[C@@H]3C)cc2)n1] | 625.6 | 5-((2R,3R)-3-(4- cyclopropylbenzamido)- 2-methylpiperidin-1- yl)-3-((4-((S)-2-methyl- 4-(oxetan-3- yl)piperazin-1- yl)phenyl)amino)pyrazine- 2-carboxamide | Similar to Example E93 |
| E-143 | 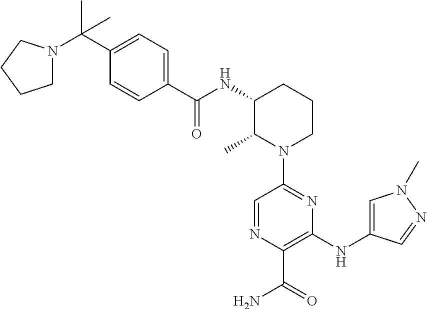 [SMILES: C[C@@H]1[C@H](NC(=O)c2ccc(C(C)(C)N3CCCC3)cc2)CCCN1c1cnc(C(N)=O)c(Nc2cnn(C)c2)n1] | 546.5 | 3-((1-methyl-1H- pyrazol-4-yl)amino)-5- ((2R,3R)-2-methyl-3- (4-(2-(pyrrolidin-1- yl)propan-2- yl)benzamido)piperidin- 1-yl)pyrazine-2- carboxamide | Similar to Example E93 |
| E-144 | 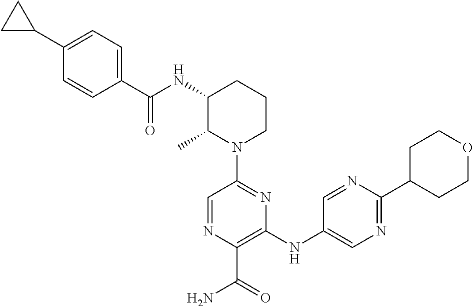 [SMILES: C[C@@H]1[C@H](NC(=O)c2ccc(C3CC3)cc2)CCCN1c1cnc(C(N)=O)c(Nc2cnc(C3CCOCC3)nc2)n1] | 557.5 | 5-((2R,3R)-3-(4- cyclopropylbenzamido)- 2-methylpiperidin-1- yl)-3-((2-(tetrahydro- 2H-pyran-4- yl)pyrimidin-5- yl)amino)pyrazine-2- carboxamide | Similar to Example E93 |
| E-145 | 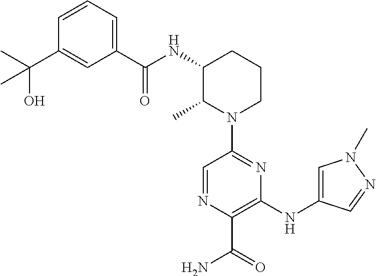 [SMILES: C[C@@H]1[C@H](NC(=O)c2cccc(C(C)(C)O)c2)CCCN1c1cnc(C(N)=O)c(Nc2cnn(C)c2)n1] | 493.4 | 5-((2R,3R)-3-(3-(2- hydroxypropan-2- yl)benzamido)-2- methylpiperidin-1-yl)- 3-((1-methyl-1H- pyrazol-4- yl)amino)pyrazine-2- carboxamide | Similar to Example E93 |
| E-146 | 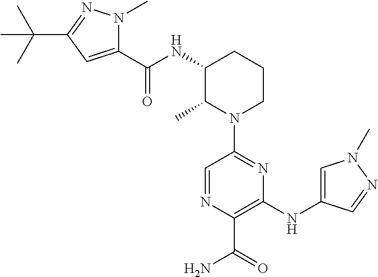 [SMILES: C[C@@H]1[C@H](NC(=O)c2cc(C(C)(C)C)nn2C)CCCN1c1cnc(C(N)=O)c(Nc2cnn(C)c2)n1] | 495.4 | 5-((2R,3R)-3-(3-(tert- butyl)-1-methyl-1H- pyrazole-5- carboxamido)-2- methylpiperidin-1-yl)- 3-((1-methyl-1H- pyrazol-4- yl)amino)pyrazine-2- carboxamide | Similar to Example E93 |
| E-147 | 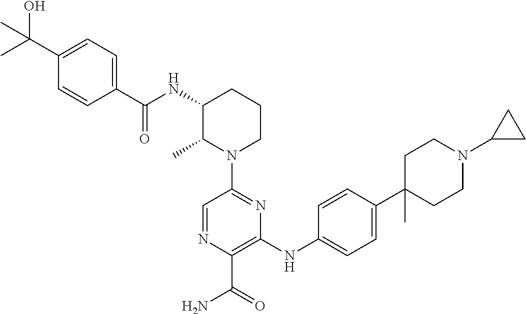 [SMILES: C[C@@H]1[C@H](NC(=O)c2ccc(C(C)(C)O)cc2)CCCN1c1cnc(C(N)=O)c(Nc2ccc(C3(C)CCN(C4CC4)CC3)cc2)n1] | 626.5 | 3-((4-(1-cyclopropyl-4- methylpiperidin-4- yl)phenyl)amino)-5- ((2R,3R)-3-(4-(2- hydroxypropan-2- yl)benzamido)-2- methylpiperidin-1- yl)pyrazine-2- carboxamide | Similar to Example E93 |
| E-148 | 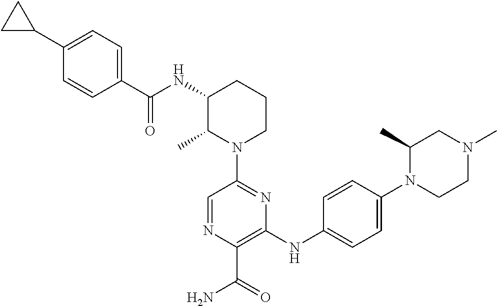 [SMILES: C[C@@H]1[C@H](NC(=O)c2ccc(C3CC3)cc2)CCCN1c1cnc(C(N)=O)c(Nc2ccc(N3CCN(C)C[C@@H]3C)cc2)n1] | 583.5 | 5-((2R,3R)-3-(4- cyclopropylbenzamido)- 2-methylpiperidin-1- yl)-3-((4-((S)-2,4- dimethylpiperazin-1- yl)phenyl)amino)pyrazine- 2-carboxamide | Similar to Example E93 |
| E-149 | 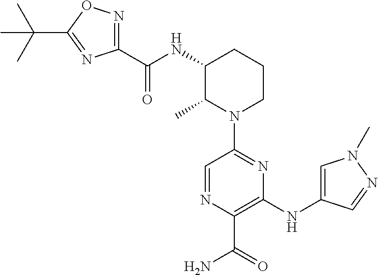 [SMILES: C[C@@H]1[C@H](NC(=O)c2noc(C(C)(C)C)n2)CCCN1c1cnc(C(N)=O)c(Nc2cnn(C)c2)n1] | 483.4 | 5-(tert-butyl)-N- ((2R,3R)-1-(5- carbamoyl-6-((1- methyl-1H-pyrazol-4- yl)amino)pyrazin-2-yl)- 2-methylpiperidin-3- yl)-1,2,4-oxadiazole-3- carboxamide | Similar to Example E93 |
| E-150 | 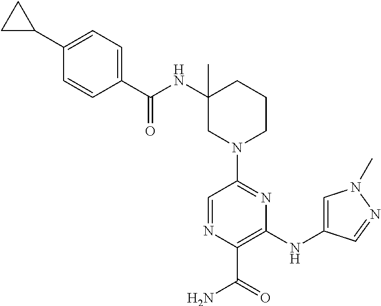 [SMILES: Cn1cc(Nc2nc(N3CCCC(C)(NC(=O)c4ccc(C5CC5)cc4)C3)cnc2C(N)=O)cn1] | 475.4 | 5-(3-(4- cyclopropylbenzamido)- 3-methylpiperidin-1- yl)-3-((1-methyl-1H- pyrazol-4- yl)amino)pyrazine-2- carboxamide | Similar to Example E93 |
| E-151 | 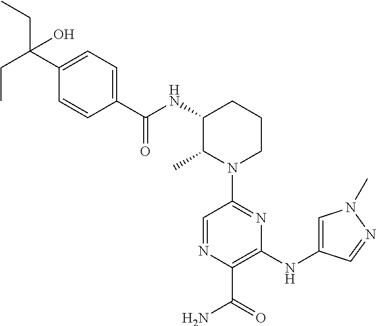 [SMILES: CCC(O)(CC)c1ccc(C(=O)N[C@@H]2CCCN(c3cnc(C(N)=O)c(Nc4cnn(C)c4)n3)[C@@H]2C)cc1] | 521.5 | 5-((2R,3R)-3-(4-(3- hydroxypentan-3- yl)benzamido)-2- methylpiperidin-1-yl)- 3-((1-methyl-1H- pyrazol-4- yl)amino)pyrazine-2- carboxamide | Similar to Example E93 |
| E-152 | 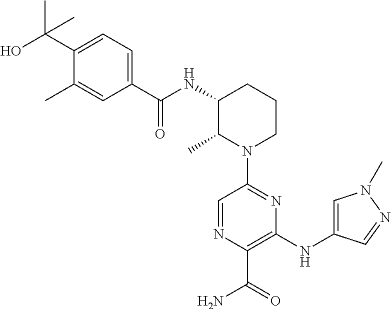 [SMILES: Cc1cc(C(=O)N[C@@H]2CCCN(c3cnc(C(N)=O)c(Nc4cnn(C)c4)n3)[C@@H]2C)ccc1C(C)(C)O] | 507.5 | 5-((2R,3R)-3-(4-(2- hydroxypropan-2-yl)-3- methylbenzamido)-2- methylpiperidin-1-yl)- 3-((1-methyl-1H- pyrazol-4- yl)amino)pyrazine-2- carboxamide | Similar to Example E93 |
| E-153 | 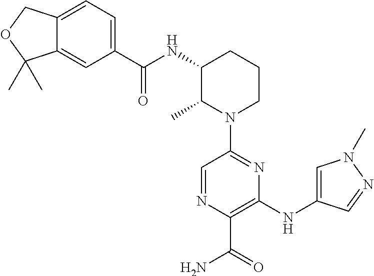 [SMILES: C[C@@H]1[C@H](NC(=O)c2ccc3c(c2)C(C)(C)OC3)CCCN1c1cnc(C(N)=O)c(Nc2cnn(C)c2)n1] | 505.5 | 5-((2R,3R)-3-(3,3- dimethyl-1,3- dihydroisobenzofuran- 5-carboxamido)-2- methylpiperidin-1-yl)- 3-((1-methyl-1H- pyrazol-4- yl)amino)pyrazine-2- carboxamide | Similar to Example E93 |
| E-154 | 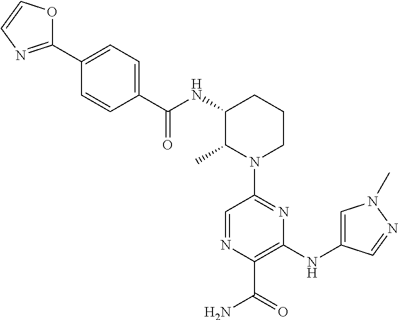 [SMILES: C[C@@H]1[C@H](NC(=O)c2ccc(-c3ncco3)cc2)CCCN1c1cnc(C(N)=O)c(Nc2cnn(C)c2)n1] | 502.4 | 3-((1-methyl-1H- pyrazol-4-yl)amino)-5- ((2R,3R)-2-methyl-3- (4-(oxazol-2- yl)benzamido)piperidin- 1-yl)pyrazine-2- carboxamide | Similar to Example E93 |
| E-155 | 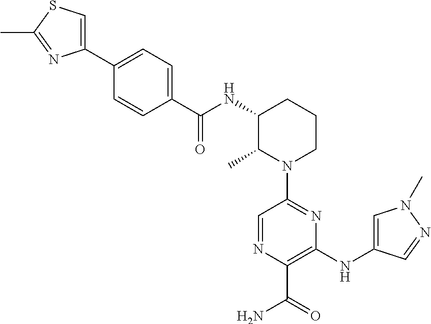 [SMILES: Cc1nc(-c2ccc(C(=O)N[C@@H]3CCCN(c4cnc(C(N)=O)c(Nc5cnn(C)c5)n4)[C@@H]3C)cc2)cs1] | 532.5 | 3-((1-methyl-1H- pyrazol-4-yl)amino)-5- ((2R,3R)-2-methyl-3- (4-(2-methylthiazol-4- yl)benzamido)piperidin- 1-yl)pyrazine-2- carboxamide | Similar to Example E93 |
| E-156 | 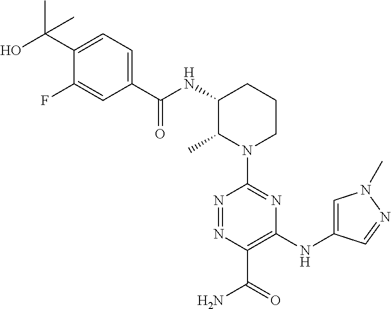 [SMILES: C[C@@H]1[C@H](NC(=O)c2ccc(C(C)(C)O)c(F)c2)CCCN1c1nnc(C(N)=O)c(Nc2cnn(C)c2)n1] | 512.5 | 3-((2R,3R)-3-(3-fluoro- 4-(2-hydroxypropan-2- yl)benzamido)-2- methylpiperidin-1-yl)- 5-((1-methyl-1H- pyrazol-4-yl)amino)- 1,2,4-triazine-6- carboxamide | Similar to Example E1 |
| E-157 | 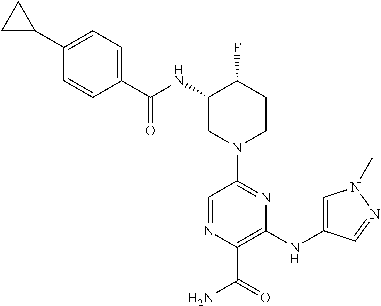 [SMILES: Cn1cc(Nc2nc(N3CC[C@@H](F)[C@@H](NC(=O)c4ccc(C5CC5)cc4)C3)cnc2C(N)=O)cn1] | 479.4 | 5-((3S,4R)-3-(4- cyclopropylbenzamido)- 4-fluoropiperidin-1- yl)-3-((1-methyl-1H- pyrazol-4- yl)amino)pyrazine-2- carboxamide | Similar to Example E93 |
| E-158 | 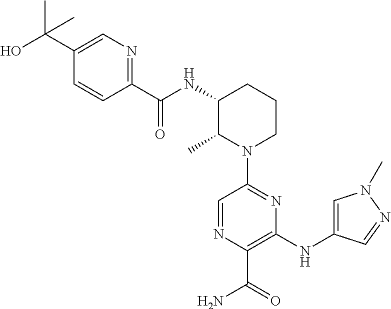 [SMILES: C[C@@H]1[C@H](NC(=O)c2ccc(C(C)(C)O)cn2)CCCN1c1cnc(C(N)=O)c(Nc2cnn(C)c2)n1] | 494.4 | 5-((2R,3R)-3-(5-(2- hydroxypropan-2- yl)picolinamido)-2- methylpiperidin-1-yl)- 3-((1-methyl-1H- pyrazol-4- yl)amino)pyrazine-2- carboxamide | Similar to Example E93 |

### Example A-92a: Btk In Vitro Inhibitory Activity (Method A)

The Btk IC₅₀s of compounds disclosed herein is determined in both a cellular kinase assay and in a cellular functional assay of BCR-induced calcium flux as described below.

Btk kinase activity is determined using a time-resolved fluorescence resonance energy transfer (TR-FRET) methodology. Measurements are performed in a reaction volume of 50 μL using 96-well assay plates. Kinase enzyme, inhibitor, ATP (at the Km for the kinase), and 1 μM peptide substrate (Biotin-AVLESEEELYSSARQ-NH₂) are incubated in a reaction buffer composed of 20 mM Tris, 50 mM NaCl, MgCl₂ (5-25 mM depending on the kinase), MnCl₂ (0-10 mM), 1 mM DTT, 0.1 mM EDTA, 0.01% bovine serum albumin, 0.005% Tween-20, and 10% DMSO at pH 7.4 for one hour. The reaction is quenched by the addition of 1.2 equivalents of EDTA (relative to divalent cation) in 25 μL of 1× Lance buffer (Perkin-Elmer). Streptavidin-APC (Perkin-Elmer) and Eu-labeled p-Tyr100 antibody (Perkin-Elmer) in 1× Lance buffer are added in a 25 μL volume to give final concentrations of 100 nM and 2.5 nM, respectively, and the mixture is allowed to incubate for one hour. The TR-FRET signal is measured on a multimode plate reader with an excitation wavelength (λ_{Ex}) of 330 nm and detection wavelengths (λ_{Em}) of 615 and 665 nm. Activity is determined by the ratio of the fluorescence at 665 nm to that at 615 nm. For each compound, enzyme activity is measured at various concentrations of compound. Negative control reactions are performed in the absence of inhibitor in replicates of six, and two no-enzyme controls are used to determine baseline fluorescence levels. Inhibition constants, Kᵢ(app), were obtained using the program BatchKᵢ(Kuzmic et al. (2000), Anal. Biochem. 286:45-50). IC₅₀s are obtained according to the equation:

IC₅₀={Ki(app)/(1+[ATP]/Kₘ^{ATP})}+[E]ₜₒₜₐ½;

For all kinases, [ATP]=Kₘ^{ATP}, [Btk]tota1=0.5 nM and [Lck]ₜₒₜₐ1=6 nM.

### Example A-92b: Btk In Vitro Inhibitory Activity (Method B)

Kinase activity is measured in vitro using electrophoretic mobility shift assay. The kinase reactions are assembled in a total volume of 25 μL in 384 well plates. The reactions comprise: BTK enzyme (1 nM, N-terminal His6-tagged, recombinant, full-length, human BTK purified from baculovirus Sf21 insect cell system), inhibitor, ATP (16 M, the apparent Kₘ for the kinase), fluorescently labeled peptide substrate (1 μM, FAM-GEEPLYWSFPAKKK-NH2) in a reaction buffer composed of 100 mM HEPES, pH7.5, 5 mM MgCl₂ 1 mM DTT, 0.1% bovine serum albumin, 0.01% Triton X-100, and 1% DMSO. The reaction is incubated for one hour and is quenched by the addition of 45 μL of termination buffer (100 mM HEPES, pH7.5, 0.01% Triton X-100, 30 mM EDTA). The terminated reactions are analyzed using 12 channel LabChip® 3000 microfluidic detection instrument (Caliper Life Sciences). The enzymatic phosphorylation of the peptide results in a change in net charge, enabling electrophoretic separation of product from substrate peptide. As substrate and product peptides are separated, two peaks of fluorescence are observed. Change in the relative fluorescence intensity of the substrate and product peaks is the parameter measured, reflecting enzyme activity. In the presence of an inhibitor, the ratio between product and substrate is altered: the signal of the product decreases, while the signal of the substrate increases.

Activity in each sample is determined as the product to sum ratio (PSR): P/(S+P), where P is the peak height of the product peptide and S is the peak height of the substrate peptide. For each compound, enzyme activity is measured at various concentrations (12 concentrations of compound spaced by 3× dilution intervals). Negative control samples (0%-inhibition in the absence of inhibitor) and positive control samples (100%-inhibition, in the presence of 20 mM EDTA) are assembled in replicates of four and are used to calculate %-inhibition values for each inhibitor at each concentration. Percent inhibition (Pᵢₙₕ) is determined using following equation:

Pᵢₙₕ=(PSR_{0%}−PSRᵢₙₕ)/(PSR_{0%}−PSR_{100%})*100,

where PSRᵢₙₕ is the product sum ratio in the presence of inhibitor, PSR_{0%} is the average product sum ration in the absence of inhibitor and PSR_{100%} is the average product sum ratio in 100%-inhibition control samples;

The IC₅₀ values of inhibitors are determined by 4 parameter sigmoidal dose-response model fitting of the inhibition curves (Pᵢₙₕ versus inhibitor concentration) using XLfit 4 software.

### Example A-92c: Btk In Vitro Inhibitory Activity (Method C)

Human Btk kinase (Genbank accession # NP_000052) was purified from insect cells as a full-length construct containing an N-terminal 6×-His tag. Btk kinase activity was determined using a radiometric filter binding assay. Measurements are performed in a low μL reaction volume 384-well assay plates. BTK enzyme (8 nM final in reaction), inhibitor (at requested doses), and 0.2 mg/mL peptide substrate (Poly-Glu-Tyr, 4:1 ratio) are incubated in a reaction buffer composed of 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO for 15 min. followed by addition of 1 μM ATP to start the assay. Kinase reactions are carried out for 120 min. at room temperature. The reaction was stopped by spotting of reaction sample onto P81 cationic exchange paper (Whatman). Unbound phosphate was removed by extensive washing of filters in 0.75% Phosphoric acid. After subtraction of background derived from control reactions containing inactive enzyme (via addition of saturating EDTA), kinase activity data for each dose of compound tested was expressed as the percent of remaining kinase activity in test samples compared to vehicle (dimethyl sulfoxide) reactions. IC₅₀ values and curve fits were obtained using Prism (GraphPad Software).

The degree of Btk inhibition was determined using one of the methods outlined in Example A-92a, 92b and 92c.

### Example A-93: Inhibition of a Panel of Kinases

The degree of inhibition of a panel of kinases is determined using the in vitro HotSpot kinase assay (purified enzymes, ³³P-ATP, an appropriate substrate and 1 μM ATP).

TABLE 10
IC₅₀ Values for Exemplary Compounds of the Invention (Formula A-I)

| Compound No. | BTK-WT (IC50) | JAK3 (IC50) | LCK (IC50) |
|---|---|---|---|
| A-1 | A | — | A |
| A-2 | A | B | A |
| A-3 | A | — | B |
| A-4 | A | B | A |
| A-5 | A | B | B |
| A-6 | A | B | B |
| A-7 | A | B | B |
| A-8 | A | B | — |
| A-9 | A | — | A |
| A-10 | A | C | B |
| A-11 | A | C | B |
| A-12 | B | C | C |
| A-13 | A | C | B |
| A-14 | A | — | B |
| A-15 | A | — | B |
| A-16 | A | — | B |
| A-17 | A | B | B |
| A-18 | A | D | D |
| A-19 | A | B | B |
| A-20 | A | D | B |
| A-21 | A | B | D |
| A-22 | A | B | B |
| A-23 | A | B | B |
| A-24 | A | D | D |
| A-25 | A | B | B |
| A-26 | A | C | D |
| A-27 | A | C | B |
| A-28 | A | C | B |
| A-29 | A | B | C |
| A-30 | A | C | D |
| A-31 | A | B | B |
| A-32 | A | C | D |
| A-33 | A | C | B |
| A-34 | A | C | C |
| A-36 | A | D | D |
| A-37 | A | D | — |
| A-38 | A | C | — |
| A-39 | A | D | — |
| A-40 | A | D | — |
| A-42 | A | — | B |
| A-43 | A | — | B |
| A-44 | A | C | D |
| A-45 | A | C | D |
| A-46 | A | C | D |
| A-47 | A | C | D |
| A-48 | A | A | A |
| A-49 | A | B | A |
| A-50 | B | D | C |
| A-51 | C | D | D |
| A-52 | A | B | A |
| A-53 | A | C | A |
| A-54 | A | C | B |
| A-55 | A | B | — |
| A-56 | A | C | — |
| A-58 | A | C | C |
| A-59 | A | C | B |
| A-60 | B | C | D |
| A-61 | B | C | B |
| A-62 | A | D | D |
| A-63 | A | D | — |
| A-64 | B | C | D |
| A-65 | C | D | — |
| A-66 | C | D | — |
| A-67 | A | B | — |
| A-68 | B | D | — |
| A-69 | B | D | — |
| A-70 | B | D | — |
| A-71 | C | C | — |
| A-72 | A | C | — |
| A-73 | A | C | — |
| A-74 | A | C | — |
| A-75 | A | A | B |
| A-76 | A | A | B |
| A-77 | A | A | B |
| A-78 | A | C | — |
| A-79 | A | C | — |
| A-80 | A | C | — |
| A-81 | A | C | — |
| A-83 | A | C | — |
| A-84 | A | A | A |
| A-85 | A | A | A |
| A-86 | A | — | — |
| A-87 | A | — | — |
| A-88 | B | — | — |
| A-89 | A | — | — |
| A-90 | A | — | — |
| A-91 | B | — | D |

IC₅₀: A ≤100 nM; 100 nM < B ≤ 1 μM; 1 μM < C ≤ 10 μM; D >10 μM

### Example B-8: Inhibition of a Panel of Kinases

The degree of inhibition of a panel of kinases is determined using the in vitro HotSpot kinase assay (purified enzymes, ³³P-ATP, an appropriate substrate and 1 μM ATP).

TABLE 11
IC₅₀ Values for Exemplary Compounds Described Herein (Formula (B-I)

| Compound No. | BTK-WT (IC50) | JAK3 (IC50) | LCK (IC50) |
|---|---|---|---|
| B-1 | A | B | A |
| B-2 | B | D | — |
| B-3 | A | A | — |
| B-4 | A | A | — |
| B-5 | A | A | B |
| B-6 | A | — | — |
| B-7 | A | — | — |
| B-8 | A | — | — |
| B-9 | A | — | — |
| B-10 | A | — | — |
| B-11 | A | — | — |
| B-12 | A | — | — |
| B-13 | A | B | A |
| B-14 | A | B | B |
| B-15 | A | A | A |
| B-16 | A | C | A |

IC₅₀: A ≤100 nM; 100 nM < B ≤ 1 μM; 1 μM < C ≤ 10 μM; D >10 μM

### Example C-34: Inhibition of a Panel of Kinases

The degree of inhibition of a panel of kinases is determined using the in vitro HotSpot kinase assay (purified enzymes, ³³P-ATP, an appropriate substrate and 1 uM ATP).

TABLE 12
IC₅₀ Values for Exemplary Compounds Described herein (Formula (C-I)

| Compound No. | BTK-WT (IC₅₀) | JAK3 (IC₅₀) | LCK (IC₅₀) |
|---|---|---|---|
| C-1 | A | A | A |
| C-2 | A | B | B |
| C-3 | A | D | D |
| C-4 | A | D | D |
| C-5 | B | B | C |
| C-6 | C | D | D |
| C-7 | D | D | D |
| C-8 | B | C | C |
| C-9 | C | D | — |
| C-10 | A | B | B |
| C-11 | A | C | C |
| C-12 | B | B | B |
| C-13 | A | C | C |
| C-14 | B | C | C |
| C-15 | B | C | C |
| C-16 | B | C | C |
| C-17 | A | — | — |
| C-18 | B | — | — |
| C-19 | B | — | — |
| C-20 | B | — | — |
| C-21 | B | — | — |
| C-22 | A | — | — |
| C-23 | A | — | — |
| C-24 | A | — | — |
| C-25 | A | — | — |
| C-26 | A | — | — |
| C-27 | A | — | — |
| C-28 | A | — | — |
| C-29 | A | A | D |
| C-30 | A | B | D |
| C-31 | A | A | B |
| C-32 | A | A | B |

IC₅₀: A ≤100 nM; 100 nM < B ≤ 1 μM; 1 μM < C ≤ 10 μM; D >10 μM

TABLE 13
IC₅₀ Values for Exemplary Compounds of the
Invention

| Compound No. | BTK-WT | JAK3 | LCK | EGFR | ITK | SRC | TEC |
|---|---|---|---|---|---|---|---|
| A-94 | B | — | — | — | C | — | — |
| A-95 | B | — | — | — | C | — | — |
| A-98 | A | D | C | D | D | C | C |
| A-99 | A | D | C | E | C | C | B |
| A-100 | A | C | C | C | C | B | C |
| A-101 | A | D | C | C | D | C | C |
| A-106 | A | C | B | C | C | C | B |
| A-107 | A | E | E | E | E | E | E |
| A-108 | C | E | E | E | E | E | C |
| A-110 | A | D | D | D | C | C | B |
| A-111 | A | C | C | D | C | C | B |
| A-115 | A | C | C | C | C | C | B |
| A-121 | A | C | A | B | B | A | A |
| A-122 | A | C | C | C | C | C | B |
| A-125 | A | C | C | C | C | B | B |
| A-127 | B | D | C | D | D | C | C |
| A-128 | A | C | B | C | C | B | B |
| A-133 | A | C | C | C | C | B | B |
| A-140 | A | D | B | C | C | B | B |
| A-141 | A | C | B | C | B | B | A |
| A-149 | A | D | B | C | B | B | B |
| A-156 | A | E | D | D | C | C | C |
| A-167 | A | E | D | E | E | D | B |
| A-169 | A | D | C | D | D | C | B |
| A-170 | A | D | B | C | C | B | B |
| A-171 | A | D | B | C | C | B | B |
| A-173 | A | D | B | C | C | B | B |
| A-174 | A | B | B | C | C | C | B |
| A-175 | A | E | E | D | E | E | C |
| A-176 | A | C | B | C | C | C | B |
| A-177 | A | D | C | D | D | C | C |
| A-184 | A | E | E | D | C | D | B |
| A-183 | A | D | B | C | B | B | B |
| A-182 | B | D | C | D | D | C | C |
| A-181 | A | C | B | D | B | B | B |
| A-180 | A | D | B | C | C | C | B |
| A-179 | A | D | C | C | C | B | B |
| A-178 | A | D | C | D | C | C | B |
| A-185 | A | D | C | E | C | B | C |
| A-186 | A | D | C | D | C | C | C |
| A-187 | C | D | E | D | E | E | D |
| A-188 | A | D | C | D | D | C | C |
| A-189 | A | D | C | D | C | C | C |
| A-190 | A | D | C | D | C | B | B |
| A-191 | B | E | D | D | E | D | D |
| A-192 | B | E | E | D | E | E | D |
| A-193 | A | C | C | C | C | C | B |

IC₅₀: A ≤10 nM; 10 nM < B ≤ 100 nM; 100 nM < C ≤ 1 μM; 1 μM < D ≤ 10 μM; E >10 μM

TABLE 14
IC₅₀ Values for Exemplary Compounds of the Invention

| Cmpd. | | | | | | | |
|---|---|---|---|---|---|---|---|
| No. | BTK-WT | ITK* | EGFR* | JAK3* | LCK* | SRC* | TEC* |
| A-198 | <10 nM | 133x | 1276x | 1276x | 112x | 69x | 3x |
| A-200 | <10 nM | 99x | 380x | 411x | 88x | 40x | 3x |
| A-201 | <10 nM | 234x | 2962x | 1102x | 49x | 47x | 8x |
| A-202 | <10 nM | 360x | 5208x | 631x | 107x | 159x | 5x |
| A-203 | <10 nM | 787x | 3304x | 7353x | 684x | 98x | 8x |
| A-205 | <10 nM | 135x | 730x | 4032x | 102x | 40x | 3x |
| A-206 | <10 nM | 157x | 896x | 341x | 63x | 33x | 1x |

*Relative to Btk IC₅₀ value.

### C Compounds

TABLE 15
IC₅₀ Values for Exemplary Compounds Described herein.

| Cmpd. | | | | | | | | |
|---|---|---|---|---|---|---|---|---|
| No. | BTK-WT | ITK | EGFR | JAK3 | LCK | SRC | TEC | TXK |
| C-33 | A | B | — | — | — | — | — | — |
| C-34 | A | B | — | — | — | — | — | — |
| C-35 | A | C | — | — | — | — | — | — |
| C-36 | A | C | — | — | — | — | — | — |
| C-37 | A | C | — | — | — | — | — | — |
| C-38 | A | A | — | — | — | — | — | — |
| C-39 | A | B | — | — | — | — | — | — |
| C-40 | A | A | A | A | C | — | A | A |
| C-41 | A | A | A | A | D | B | A | A |
| C-42 | A | B | — | B | D | C | A | A |
| C-43 | A | B | — | — | — | — | — | — |
| C-44 | A | A | A | A | A | A | A | A |
| C-45 | A | A | — | — | — | — | — | — |
| C-46 | A | C | — | — | — | — | — | — |
| C-47 | A | B | — | — | — | — | — | — |
| C-48 | A | A | — | — | — | — | — | — |
| C-49 | A | D | — | — | — | — | — | — |
| C-50 | A | C | — | — | — | — | — | — |
| C-51 | A | B | A | — | — | — | A | A |
| C-52 | A | B | — | — | — | — | A | A |
| C-53 | A | B | — | — | — | — | A | A |
| C-54 | A | B | — | — | — | — | A | A |
| C-55 | A | B | — | — | — | — | — | — |
| C-56 | A | A | A | A | B | B | A | A |
| C-57 | A | A | A | A | B | B | A | A |
| C-58 | A | A | A | — | — | — | A | A |
| C-59 | A | A | A | — | — | — | A | A |
| C-60 | A | A | — | — | — | — | A | A |
| C-61 | A | A | — | — | — | — | A | A |
| C-62 | A | B | — | — | — | — | A | A |
| C-63 | A | A | — | — | — | — | A | A |
| C-64 | A | A | — | — | — | — | A | A |
| C-65 | A | A | — | — | — | — | A | A |
| C-66 | A | A | — | — | — | — | | |
| C-67 | A | A | — | — | — | — | A | E |
| C-68 | A | A | — | — | — | — | | |
| C-69 | A | C | — | — | — | — | A | A |
| C-70 | A | B | — | — | — | — | A | A |
| C-71 | A | B | B | — | — | — | A | A |
| C-72 | A | B | — | — | — | — | A | B |
| C-73 | A | A | — | — | — | — | — | — |
| C-74 | A | B | — | — | — | — | — | — |
| C-75 | A | B | B | — | — | — | A | A |
| C-76 | A | A | B | — | — | — | A | A |
| C-77 | A | B | A | — | — | — | A | A |
| C-78 | A | B | A | — | — | — | A | A |
| C-79 | A | B | — | — | — | — | A | A |
| C-80 | A | A | — | — | — | — | A | A |
| C-81 | A | B | — | — | — | — | A | A |
| C-82 | A | A | — | — | — | — | A | A |
| C-83 | A | B | — | — | — | — | A | A |
| C-84 | A | A | A | A | B | B | A | A |
| C-85 | A | A | B | A | C | B | A | A |
| C-86 | A | B | — | — | — | — | — | — |
| C-87 | A | A | — | — | — | — | — | — |

IC₅₀: A ≤10 nM; 10 nM < B ≤ 100 nM; 100 nM < C ≤ 1 μM; 1 μM < D ≤ 10 μM; E >10 μM

### D-Compounds: Inhibition of a Panel of Kinases

The degree of inhibition of a panel of kinases is determined using the in vitro HotSpot kinase assay (purified enzymes, ³³P-ATP, an appropriate substrate and 1 μM ATP).

### D-Compounds (Panel Data)

TABLE 16
IC₅₀ Values for Exemplary Compounds Described herein.

| Cmpd. | BTK- | | | | | | |
|---|---|---|---|---|---|---|---|
| No. | WT | EGFR | ITK | JAK3 | LCK | SRC | TEC |
| D-1 | A | D | C | D | D | C | B |
| D-2 | C | E | E | D | E | D | D |
| D-3 | E | E | E | E | E | E | E |
| D-4 | B | E | D | D | E | E | C |
| D-5 | A | C | C | C | C | B | B |
| D-6 | A | C | C | D | B | B | B |
| D-7 | A | C | B | D | B | B | B |
| D-8 | B | E | E | E | E | E | D |
| D-9 | A | B | A | C | A | A | B |
| D-10 | A | C | C | D | C | C | B |
| D-11 | A | C | C | D | B | B | B |
| D-12 | A | C | C | C | B | C | B |
| D-13 | A | D | D | D | C | C | C |
| D-14 | A | D | C | D | B | B | B |
| D-15 | A | C | B | D | B | B | B |
| D-16 | A | C | C | D | B | C | B |
| D-17 | A | D | C | D | C | C | C |
| D-18 | A | D | C | D | C | B | B |
| D-19 | A | C | C | C | C | C | B |
| D-20 | A | D | D | D | C | C | B |
| D-21 | A | D | C | D | C | B | B |
| D-22 | A | D | D | E | D | C | B |
| D-23 | A | D | C | C | C | B | A |
| D-24 | A | D | C | D | B | C | A |
| D-25 | B | D | D | D | C | C | B |
| D-26 | B | E | D | E | D | D | C |
| D-27 | A | C | C | C | C | B | A |
| D-28 | D | E | D | E | E | D | E |
| D-29 | C | E | D | E | E | D | D |
| D-30 | A | E | D | E | C | C | B |
| D-31 | B | E | D | D | D | C | C |
| D-32 | C | E | E | E | D | E | C |
| D-33 | A | C | B | D | B | B | B |
| D-34 | D | E | E | E | E | E | D |
| D-35 | A | D | D | C | C | B | B |
| D-36 | A | C | C | C | C | C | A |
| D-37 | B | C | D | | C | D | B |
| D-38 | A | C | B | B | B | B | B |
| D-39 | A | E | C | C | C | C | B |
| D-40 | A | C | C | C | B | B | A |
| D-41 | A | D | C | C | B | B | B |
| D-42 | C | E | E | E | E | E | C |
| D-43 | A | D | C | C | C | C | B |
| D-44 | B | C | E | D | C | D | C |
| D-45 | B | E | D | D | C | D | C |
| D-46 | B | C | D | D | C | C | B |
| D-47 | D | E | E | E | E | D | D |
| D-48 | B | E | E | D | E | C | B |
| D-49 | B | E | E | E | E | E | C |
| D-50 | B | E | D | D | D | C | C |
| D-51 | A | C | B | C | B | A | A |
| D-52 | B | D | C | E | D | C | C |
| D-53 | B | D | C | D | C | C | B |
| D-54 | B | E | D | D | C | C | C |
| D-55 | A | D | C | E | B | B | A |
| D-56 | A | C | C | E | B | B | A |
| D-57 | A | D | C | D | C | B | B |
| D-58 | A | C | D | D | C | C | C |
| D-59 | C | D | D | E | D | D | E |
| D-60 | C | D | C | D | D | C | D |
| D-61 | C | E | D | E | D | D | E |
| D-62 | C | E | E | E | E | E | C |
| D-63 | D | E | E | E | E | E | E |
| D-64 | D | E | E | E | E | E | D |
| D-65 | C | E | E | E | E | D | D |
| D-66 | A | B | B | C | A | A | A |
| D-67 | B | C | D | D | C | C | C |
| D-68 | D | E | E | D | E | D | E |
| D-69 | B | C | D | C | B | C | D |
| D-70 | B | C | C | C | C | C | C |
| D-71 | B | C | D | B | B | C | C |
| D-72 | B | D | D | D | B | C | D |
| D-73 | A | D | C | E | D | C | B |
| D-74 | C | C | E | C | C | D | D |
| D-75 | B | C | D | C | C | C | C |
| D-76 | C | D | E | D | E | D | D |
| D-77 | C | D | E | D | D | D | D |
| D-78 | C | C | D | D | C | C | D |
| D-79 | C | D | E | D | D | D | E |
| D-80 | B | C | E | D | C | C | D |
| D-81 | A | C | C | D | B | B | B |
| D-82 | A | C | C | D | B | B | B |
| D-83 | B | E | D | D | D | C | B |
| D-84 | A | D | C | E | C | B | A |
| D-85 | A | C | C | C | C | C | A |
| D-86 | A | D | B | C | B | B | A |
| D-87 | A | C | C | C | C | B | B |
| D-88 | A | D | C | D | C | B | B |
| D-89 | A | C | C | C | B | B | B |
| D-90 | B | D | C | D | C | C | B |
| D-91 | A | C | C | C | B | B | B |
| D-92 | A | D | D | E | C | C | B |
| D-93 | B | D | C | C | C | B | B |
| D-94 | A | D | C | C | C | B | B |
| D-95 | A | C | C | D | C | C | B |
| D-96 | B | E | E | D | E | E | B |
| D-97 | B | E | D | D | E | C | B |
| D-98 | A | E | D | D | E | E | C |
| D-99 | A | D | C | C | C | C | A |
| D-100 | A | D | C | D | C | B | A |
| D-101 | A | C | B | D | B | B | A |
| D-102 | A | C | B | C | B | B | B |
| D-103 | A | C | C | E | C | D | C |
| D-104 | A | C | B | C | B | B | A |
| D-105 | A | C | B | C | B | B | A |
| D-106 | A | C | B | C | B | B | A |
| D-107 | A | D | C | D | C | B | A |
| D-108 | A | D | C | E | C | B | B |
| D-109 | A | D | C | D | C | C | B |
| D-110 | A | D | D | D | C | C | C |
| D-111 | A | C | C | C | C | B | C |
| D-112 | A | C | B | C | A | A | A |
| D-113 | A | C | C | C | B | C | B |
| D-114 | B | E | E | E | E | C | B |
| D-115 | A | C | C | C | C | C | B |
| D-116 | A | D | D | D | B | C | C |
| D-117 | B | D | D | D | C | C | C |
| D-118 | A | D | C | D | C | C | C |
| D-119 | C | C | E | E | D | D | E |
| D-120 | C | E | D | D | D | D | E |
| D-121 | B | D | D | D | C | C | D |
| D-122 | A | D | C | D | B | B | B |
| D-123 | A | D | C | E | C | C | B |
| D-124 | B | E | E | E | E | E | D |
| D-125 | A | E | D | D | C | D | D |
| D-126 | B | E | D | E | C | C | D |
| D-127 | C | E | D | E | D | D | D |
| D-128 | A | D | C | D | C | C | B |
| D-129 | C | D | E | E | E | E | E |
| D-130 | A | D | C | E | D | C | C |
| D-131 | B | C | D | C | C | C | C |
| D-132 | B | E | D | E | D | E | D |
| D-133 | A | D | C | D | C | C | C |
| D-134 | A | C | B | C | B | B | B |
| D-135 | A | D | C | D | C | C | B |
| D-136 | B | C | D | D | C | C | C |
| D-137 | B | D | E | E | D | D | D |
| D-138 | A | C | C | B | B | C | B |
| D-139 | A | D | E | E | E | E | C |
| D-140 | A | D | B | C | B | B | B |
| D-141 | A | D | C | D | C | C | B |
| D-142 | A | D | D | C | B | B | B |
| D-143 | B | D | E | E | D | D | C |
| D-144 | C | E | E | E | D | D | D |
| D-145 | B | D | E | E | D | D | D |
| D-146 | A | C | B | D | B | B | B |
| D-147 | B | D | E | D | C | C | C |
| D-148 | B | D | D | D | C | C | C |
| D-149 | B | E | C | D | C | C | C |
| D-150 | A | C | C | D | B | B | A |
| D-151 | B | D | D | E | B | C | B |
| D-152 | B | D | D | D | D | C | B |
| D-153 | A | C | B | C | B | B | A |
| D-154 | A | E | D | C | C | C | C |
| D-155 | A | D | C | C | C | C | B |
| D-156 | A | E | C | D | D | C | B |
| D-157 | A | D | B | C | B | B | A |
| D-158 | A | D | C | D | C | C | B |
| D-159 | A | C | C | C | B | B | A |
| D-160 | A | C | C | C | B | B | A |
| D-161 | A | D | C | D | C | B | A |
| D-162 | A | C | C | D | B | B | A |
| D-163 | A | C | B | C | A | A | B |
| D-164 | B | C | D | C | C | C | C |
| D-165 | A | E | C | D | C | C | B |
| D-166 | A | E | E | E | E | E | E |
| D-167 | A | D | C | C | C | C | B |
| D-168 | C | E | E | E | E | E | C |
| D-169 | A | C | C | C | C | C | B |
| D-170 | B | C | E | D | B | C | C |
| D-171 | B | E | E | C | C | C | C |
| D-172 | B | B | E | C | C | C | C |
| D-173 | A | C | C | C | B | B | B |
| D-174 | A | C | C | C | C | B | B |
| D-175 | B | D | D | D | D | C | D |
| D-176 | A | E | C | C | B | B | B |
| D-177 | A | C | B | C | B | B | A |
| D-178 | B | E | D | E | D | D | D |
| D-179 | C | E | D | E | E | E | D |
| D-180 | C | D | E | E | E | E | E |
| D-181 | A | C | D | C | C | B | B |
| D-182 | A | C | C | C | B | B | C |
| D-183 | A | C | B | C | B | A | B |
| D-184 | A | E | E | E | D | D | B |
| D-185 | A | C | C | D | B | B | B |
| D-186 | B | D | E | D | D | C | C |
| D-187 | B | D | D | D | C | C | C |
| D-188 | A | C | C | D | C | B | B |
| D-189 | C | D | E | D | E | E | D |
| D-190 | A | D | D | D | C | C | C |
| D-191 | B | E | D | D | C | C | C |
| D-192 | D | E | D | D | E | D | D |
| D-193 | B | E | E | D | E | D | C |
| D-194 | A | D | C | D | C | C | A |
| D-195 | A | E | C | D | C | C | B |
| D-196 | A | C | B | C | B | B | B |
| D-197 | A | D | C | D | C | C | B |
| D-198 | A | D | C | C | B | B | B |
| D-199 | D | E | E | D | E | D | D |
| D-200 | C | D | E | E | D | D | D |
| D-201 | A | D | D | D | C | B | A |
| D-202 | B | E | D | E | B | C | C |
| D-203 | A | C | B | D | B | B | A |
| D-205 | A | C | C | D | B | B | A |
| D-206 | A | D | C | D | B | B | A |
| D-207 | A | E | D | C | C | C | C |
| D-208 | A | D | C | D | C | B | B |
| D-209 | B | E | D | D | C | C | B |
| D-210 | A | D | C | D | C | C | A |
| D-211 | A | D | C | C | B | B | A |
| D-212 | A | D | C | C | C | B | A |
| D-213 | A | D | C | D | C | B | A |
| D-214 | A | C | C | D | B | B | A |
| D-215 | A | E | D | E | D | C | C |
| D-216 | A | D | C | C | C | B | A |
| D-217 | A | D | C | C | C | B | A |
| D-218 | A | D | C | D | C | B | A |
| D-219 | A | D | C | D | B | B | A |
| D-220 | D | E | E | D | E | D | D |
| D-221 | A | D | D | E | C | C | B |
| D-222 | A | E | E | E | D | D | B |
| D-223 | A | D | D | E | C | C | B |
| D-224 | A | C | C | D | C | B | A |
| D-225 | A | D | C | C | C | B | A |
| D-226 | A | C | B | C | B | A | A |
| D-227 | A | C | B | C | B | A | A |
| D-228 | A | D | C | D | C | B | A |
| D-229 | A | D | D | E | B | C | B |
| D-230 | A | D | D | E | C | C | B |
| D-231 | A | D | C | E | C | B | B |
| D-232 | A | D | C | D | B | B | A |
| D-233 | A | C | C | D | C | B | B |
| D-234 | A | C | B | C | B | B | A |
| D-235 | A | C | C | C | C | B | A |
| D-236 | A | C | C | C | B | B | A |
| D-237 | A | D | C | C | B | B | A |
| D-238 | A | C | C | C | B | B | A |
| D-239 | A | C | C | C | B | B | A |
| D-240 | A | C | C | D | C | C | B |
| D-241 | A | C | D | D | C | B | B |
| D-242 | A | D | C | C | C | B | A |
| D-243 | A | D | C | C | B | B | A |
| D-244 | A | D | C | C | C | B | B |
| D-245 | A | D | C | C | C | B | A |
| D-246 | A | D | C | D | B | B | A |
| D-247 | A | D | C | D | C | C | B |
| D-248 | A | C | C | C | B | B | A |
| D-249 | A | D | C | D | B | B | A |
| D-250 | A | C | C | C | B | B | A |
| D-251 | A | D | C | D | B | B | A |
| D-252 | A | C | B | C | B | B | A |
| D-253 | A | C | C | C | C | B | A |
| D-254 | A | D | C | D | C | B | A |
| D-255 | A | D | C | C | C | B | A |
| D-256 | A | C | C | C | B | B | A |
| D-257 | A | C | C | C | B | B | A |
| D-258 | A | D | C | D | C | B | A |
| D-259 | A | D | C | D | B | B | A |
| D-260 | A | D | C | D | B | B | A |
| D-261 | A | C | C | D | B | B | A |
| D-262 | A | D | C | D | B | B | A |
| D-263 | A | C | C | D | B | B | A |
| D-264 | A | C | C | D | B | B | A |
| D-265 | A | C | C | C | C | B | A |
| D-266 | A | D | B | D | B | B | A |
| D-267 | A | C | C | D | B | B | A |
| D-268 | A | C | C | D | B | B | A |
| D-269 | A | E | C | D | C | B | C |
| D-270 | A | D | C | D | C | C | C |
| D-271 | B | D | E | E | D | D | D |
| D-272 | B | D | E | E | E | E | D |
| D-273 | A | C | C | D | C | C | B |
| D-274 | A | D | C | D | C | B | A |
| D-275 | A | E | D | E | D | C | B |
| D-276 | D | E | E | E | E | E | E |
| D-277 | A | D | C | C | B | B | A |
| D-278 | E | E | E | E | E | E | E |
| D-279 | A | D | C | E | C | C | A |
| D-280 | A | D | B | D | A | A | A |
| D-281 | A | D | C | D | C | C | B |
| D-282 | A | D | C | E | C | C | B |
| D-283 | A | D | C | D | B | B | A |
| D-284 | A | E | C | E | B | B | A |
| D-285 | A | D | B | C | B | B | A |
| D-286 | A | D | B | D | C | B | B |
| D-287 | A | E | C | D | C | C | A |
| D-288 | A | D | C | D | C | B | A |
| D-289 | A | C | B | D | B | A | A |
| D-290 | B | D | E | E | D | D | C |
| D-291 | A | C | B | D | B | B | B |
| D-292 | A | C | B | D | B | B | A |
| D-293 | A | E | D | D | E | B | B |
| D-294 | A | C | B | C | B | B | A |
| D-295 | A | C | C | C | B | B | A |
| D-296 | A | C | C | D | C | B | B |
| D-297 | A | C | B | C | B | B | A |
| D-298 | A | D | B | C | B | B | A |
| D-299 | B | E | E | E | E | D | B |
| D-300 | E | E | E | E | E | E | E |
| D-301 | A | C | C | D | C | C | B |
| D-302 | A | B | B | C | B | B | A |
| D-303 | B | E | D | E | E | D | B |
| D-304 | A | D | D | D | C | C | B |
| D-305 | A | D | C | E | C | B | B |
| D-306 | A | D | C | D | C | C | B |
| D-307 | A | E | D | E | C | C | B |
| D-308 | A | D | B | D | B | B | A |
| D-309 | A | C | C | C | C | B | A |
| D-310 | A | D | B | C | B | B | A |
| D-311 | A | D | B | C | B | B | B |
| D-312 | A | C | B | C | A | A | A |
| D-313 | A | C | B | C | B | B | A |
| D-314 | A | D | C | D | C | B | B |
| D-315 | D | E | E | E | E | E | D |
| D-316 | A | C | C | D | B | B | B |
| D-317 | A | E | E | D | E | E | B |
| D-318 | A | E | C | E | C | C | B |
| D-319 | A | D | C | E | C | B | A |
| D-320 | A | C | B | D | B | B | A |
| D-321 | B | E | C | E | D | C | B |

IC₅₀: A ≤10 nM; 10 nM < B ≤ 100 nM; 100 nM < C ≤ 1 μM; 1 μM < D ≤ 10 μM; E >10 μM

### E Compounds

TABLE 17
IC₅₀ Values for Exemplary Compounds Described herein.

| Ex. # | BTK-WT | EGFR | ITK | JAK3 | LCK | SRC | TEC |
|---|---|---|---|---|---|---|---|
| E-1 | A | — | A | A | A | — | A |
| E-2 | A | — | A | A | A | — | A |
| E-5 | A | — | C | A | C | — | A |
| E-6 | A | — | B | A | B | — | A |
| E-7 | A | — | B | B | C | — | A |
| E-10 | A | B | A | D | A | A | B |
| E-11 | A | A | A | A | B | B | A |
| E-12 | A | B | A | A | C | B | A |
| E-13 | B | — | C | — | — | — | C |
| E-14 | A | C | C | B | C | C | B |
| E-16 | A | — | A | — | — | — | A |
| E-17 | A | — | B | — | — | — | — |
| E-18 | A | — | B | — | — | — | — |
| E-19 | A | — | C | — | — | — | — |
| E-20 | A | — | A | — | — | — | — |
| E-21 | B | — | C | — | — | — | — |
| E-22 | B | — | C | — | — | — | — |
| E-23 | A | — | B | — | — | — | — |
| E-24 | A | A | A | A | C | — | A |
| E-25 | A | — | C | — | — | — | — |
| E-26 | A | A | A | A | D | B | A |
| E-27 | A | — | B | — | — | — | — |
| E-28 | A | — | C | — | — | — | — |
| E-29 | A | — | C | — | — | — | — |
| E-32 | A | — | A | — | — | — | — |
| E-34 | B | — | C | — | — | — | — |
| E-35 | A | — | C | — | — | — | — |
| E-36 | A | — | B | — | — | — | — |
| E-37 | A | — | D | — | — | — | — |
| E-38 | A | — | A | — | — | — | — |
| E-39 | A | — | C | — | — | — | — |
| E-40 | A | A | B | A | C | C | A |
| E-41 | A | B | B | A | C | C | A |
| E-42 | A | — | B | — | — | — | A |
| E-43 | A | — | B | — | — | — | A |
| E-44 | A | — | B | — | — | — | — |
| E-45 | A | A | A | A | B | B | A |
| E-46 | A | A | A | — | — | — | A |
| E-47 | A | A | A | — | — | — | A |
| E-48 | A | — | A | — | — | — | A |
| E-49 | A | — | A | — | — | — | A |
| E-50 | A | — | B | — | — | — | A |
| E-51 | A | — | A | — | — | — | A |
| E-52 | A | — | A | — | — | — | A |
| E-53 | A | — | C | — | — | — | A |
| E-54 | A | — | B | — | — | — | A |
| E-55 | A | B | B | — | — | — | A |
| E-56 | A | — | B | — | — | — | A |
| E-57 | A | — | A | — | — | — | — |
| E-58 | A | — | C | — | — | — | — |
| E-59 | A | — | C | — | — | — | — |
| E-60 | A | — | B | — | — | — | — |
| E-61 | A | B | B | — | C | — | A |
| E-62 | A | B | A | — | — | — | A |
| E-63 | A | A | B | — | — | — | A |
| E-64 | A | A | B | — | — | — | A |
| E-65 | A | — | B | — | — | — | A |
| E-66 | A | — | A | — | — | — | A |
| E-67 | A | B | B | — | C | — | A |
| E-68 | A | A | A | — | C | — | A |
| E-69 | A | — | B | — | — | — | A |
| E-70 | A | — | B | — | — | — | — |
| E-71 | A | — | A | — | — | — | — |
| E-72 | B | — | D | — | — | — | — |
| E-73 | A | — | A | — | — | — | A |
| E-74 | A | — | B | — | — | — | A |
| E-75 | A | — | B | — | — | — | A |
| E-76 | A | — | B | — | — | — | A |
| E-77 | A | — | B | — | — | — | A |
| E-78 | A | — | B | — | — | — | A |
| E-79 | A | — | B | — | — | — | A |
| E-80 | A | — | A | — | — | — | A |
| E-81 | A | — | C | — | — | — | A |
| E-82 | A | — | B | — | — | — | A |
| E-83 | A | — | B | — | — | — | A |
| E-84 | A | — | B | — | — | — | A |
| E-85 | A | — | B | — | — | — | A |
| E-86 | A | — | B | — | — | — | A |
| E-87 | A | — | C | — | — | — | A |
| E-88 | A | — | C | — | — | — | B |
| E-89 | C | — | D | — | — | — | C |
| E-90 | A | — | B | — | — | — | A |
| E-91 | B | — | D | — | — | — | C |
| E-92 | C | — | D | — | — | — | |
| E-93 | A | B | B | A | B | A | A |
| E-94 | A | — | C | — | — | — | — |
| E-95 | A | — | B | A | A | — | A |
| E-96 | A | — | A | A | A | — | A |
| E-97 | A | B | B | A | C | A | A |
| E-98 | A | C | C | — | — | — | A |
| E-99 | A | A | A | — | B | — | A |
| E-100 | A | A | A | — | B | — | A |
| E-101 | A | — | C | — | — | — | — |
| E-102 | A | — | B | B | D | C | A |
| E-103 | A | A | A | A | A | A | A |
| E-104 | A | A | A | A | B | B | A |
| E-105 | A | — | A | — | — | — | |
| E-106 | A | A | A | A | B | A | A |
| E-107 | A | — | A | — | — | — | |
| E-108 | A | A | A | A | B | B | A |
| E-109 | A | B | A | A | C | B | A |
| E-110 | A | C | A | A | D | C | A |
| E-111 | A | — | A | — | — | — | A |
| E-112 | A | — | B | — | — | — | A |
| E-113 | A | — | B | — | — | — | A |
| E-114 | A | — | A | — | — | — | A |
| E-115 | A | A | A | — | — | — | A |
| E-116 | A | B | A | — | — | — | A |
| E-117 | A | B | A | — | — | — | A |
| E-118 | A | B | A | — | — | — | A |
| E-119 | A | B | A | — | — | — | A |
| E-120 | A | — | A | — | — | — | A |
| E-121 | A | A | A | A | B | B | A |
| E-122 | A | — | A | — | — | — | A |
| E-123 | A | — | A | A | B | B | A |
| E-124 | A | — | A | A | B | A | A |
| E-125 | A | B | A | A | B | A | A |
| E-126 | A | B | A | — | — | — | A |
| E-127 | A | — | A | A | A | A | A |
| E-128 | C | — | B | D | B | B | D |
| E-136 | A | C | B | C | B | B | A |
| E-137 | A | D | B | D | B | B | B |
| E-138 | A | C | C | C | C | B | A |
| E-139 | A | C | C | C | B | B | A |
| E-140 | C | E | E | E | D | E | D |
| E-141 | C | C | D | D | C | C | C |
| E-142 | A | D | B | D | B | B | A |
| E-143 | A | E | C | C | B | B | A |
| E-144 | B | E | C | E | C | C | C |
| E-145 | A | C | C | C | B | B | A |
| E-146 | A | D | C | D | B | B | A |
| E-147 | A | C | B | C | A | A | A |
| E-148 | A | C | B | C | B | B | A |
| E-149 | C | D | D | D | D | D | C |
| E-150 | C | E | E | D | D | D | C |
| E-151 | A | C | C | C | C | B | A |
| E-152 | A | D | C | C | B | B | A |
| E-153 | A | D | C | D | B | B | A |
| E-154 | A | D | C | C | B | B | A |
| E-155 | A | C | C | C | B | B | A |

IC₅₀: A ≤10 nM; 10 nM < B ≤ 100 nM; 100 nM < C ≤ 1 μM; 1 μM < D ≤ 10 μM; E >10 μM

### Example F-1: Pharmaceutical Compositions

The compositions described below are presented with a compound described herein for illustrative purposes.

### Example F-1a: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example F-1b: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example F-1c: Sublingual (Hard Lozenge) Composition

To prepare a pharmaceutical composition for buccal delivery, such as a hard lozenge, mix 100 mg of a compound of described herein with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example F-1d: Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example F-1e: Rectal Gel Composition

To prepare a pharmaceutical composition for rectal delivery, 100 mg of a compound described herein is mixed with 2.5 g of methylcellulose (1500 mPa), 100 mg of methylparapen, 5 g of glycerin and 100 mL of purified water. The resulting gel mixture is then incorporated into rectal delivery units, such as syringes, which are suitable for rectal administration.

### Example F-1f: Topical Gel Composition

To prepare a pharmaceutical topical gel composition, 100 mg of a compound described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example F-1g: Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example F-2: Clinical Trial of the Safety and Efficacy of a Compound Described Herein in Rheumatoid Arthritis Patients

The purpose of this study is to determine the safety and efficacy of a compound described herein in patients with rheumatoid arthritis.

### Inclusion Criteria

Adult males/Females aged 18-80 years.

Patients who are taking NSAIDs for the treatment of rheumatoid arthritis.

Patients who belong to ACR functional class 1, 2, 3.

### Exclusion Criteria

Patients who belong to ACR functional class 4.

Patients who are hypersensitive to clinical trial medicines or excipient.

Patients who have experience of Cerebrovascular bleeding, bleeding disorder.

### Study Design

Allocation: Randomized, placebo-controlled.

Intervention Model: Single Group Assignment.

Masking: Double Blind (Subject, Caregiver).

Primary Purpose: Supportive Care.

### Primary Outcome Measures

Changes in ‘100 mm pain VAS’ value from baseline [Time Frame: −14, 0, 14, 28, 42 day] [Designated as safety issue: No].

Determine PK of an orally administered compound described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (I) having the structure: 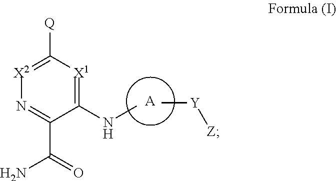
wherein:
Q is 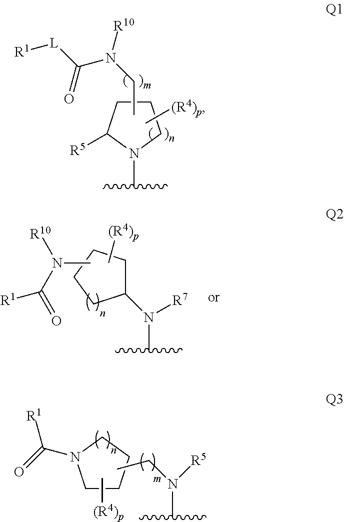
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
L is a bond, or is NR¹¹;
R¹ is substituted or unsubstituted C₂-C₄alkenyl, substituted or unsubstituted C₂-C₄alkynyl, substituted or unsubstituted cyclohexyl, substituted or unsubstituted C₂-C₇heterocycloalkyl, substituted or unsubstituted C₆-C₁₂aryl, or substituted or unsubstituted C₁-C₁₂heteroaryl; or R¹ is substituted or unsubstituted isoindolinyl or CN; or R¹ and R¹⁰ together with the -L-C(O)—N— between them form a substituted or unsubstituted C₁-C₁₂heteroaryl or a substituted or unsubstituted C₂-C₇heterocycloalkyl optionally fused with a substituted or unsubstituted phenyl ring, which C₂-C₇heterocycloalkyl is other than 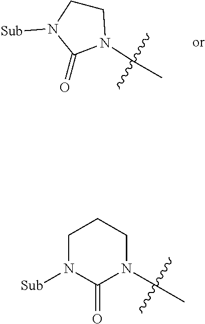
(wherein Sub represents H or a substituent); and further wherein, when m is 1, R¹ may also be substituted or unsubstituted C₁-C₄alkyl;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl:
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or two R⁴ form a C₁-C₄alkylene;
R⁵ is H, halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂; or R⁵ together with one R⁴ form a C₁-C₄alkylene;
R¹⁰ and R¹¹ are independently H, or substituted or unsubstituted C₁-C₄alkyl; or R¹⁰ and R¹¹ connect to form a C₁-C₄alkylene;
m is 0 or 1;
n is 0, 1, 2 or 3; and
p is 0, 1, 2 or 3;
or a pharmaceutically acceptable solvate, pharmaceutically acceptable salt, and/or pharmaceutically acceptable prodrug thereof;
provided that:
(1) when Q is Q1, m is 0, R¹ is substituted or unsubstituted C₂-C₄alkenyl, and X¹ is N, then X² is other than C(Et);
(2) when Q is Q1, and m is 0, then -A-Y—Z is other than 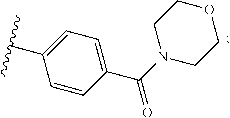 [SMILES: *c1ccc(C(=O)N2CCOCC2)cc1]
(3) when Q is Q1, and m is 0, then R¹ is other than 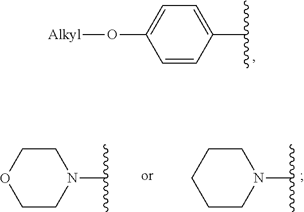
(4) when Q is Q2, R¹ is substituted or unsubstituted C₂-C₄alkenyl, A is substituted or unsubstituted phenyl, R⁷ is H, the group 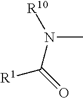
and the group 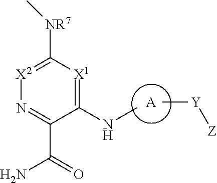
are attached to the same carbon atom or attached to carbon atoms that are adjacent to each other, and X¹ is N, then X² is other than CH or C(Et);
(5) when Q is Q3, and m is 0, then -A-Y—Z is other than 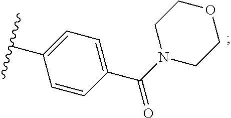 [SMILES: *c1ccc(C(=O)N2CCOCC2)cc1]
(6) when Q is Q3, m is 0, A is quinolinyl, X¹ is N and X² is CH, then R¹ is other than Me;
(7) when Q is Q3, R¹ is substituted or unsubstituted C₁-C₄alkyl or substituted or unsubstituted C₂-C₄alkenyl, m is 0, and X¹ is N, then X² is CH or N; and
(8) the compound is other than:
(R)-3-(3-(4-tert-butylbenzamido)piperidin-1-yl)-5-(5-fluoropyridin-3-ylamino)-1,2,4-triazine-6-carboxamide;
(R)-3-(3-(4-tert-butylbenzamido)piperidin-1-yl)-5-(p-tolylamino)-1,2,4-triazine-6-carboxamide;
(R)-3-(3-(4-tert-butylbenzamido)piperidin-1-yl)-5-(m-tolylamino)-1,2,4-triazine-6-carboxamide;
(R)-3-(3-(4-tert-butylbenzamido)piperidin-1-yl)-5-(4-(methylsulfonyl)phenylamino)-1,2,4-triazine-6-carboxamide;
(R)-3-(3-(4-tert-butylbenzamido)piperidin-1-yl)-5-(4-(pyrimidin-2-yl)phenylamino)-1,2,4-triazine-6-carboxamide;
(R)-3-(3-(4-tert-butylbenzamido)piperidin-1-yl)-5-(3-(pyrimidin-2-yl)phenylamino)-1,2,4-triazine-6-carboxamide;
(R)-3-(3-(4-tert-butylbenzamido)piperidin-1-yl)-5-(4-(oxazol-2-yl)phenylamino)-1,2,4-triazine-6-carboxamide;
(R)-5-(3-(4-tert-butylbenzamido)piperidin-1-yl)-3-(3-methylisothiazol-5-ylamino)picolinamide;
(R)-5-(3-(4-tert-butylbenzamido)piperidin-1-yl)-3-(4-(4-methylpiperazin-1-yl)phenylamino)pyrazine-2-carboxamide;
(R)-5-(3-(4-tert-butylbenzamido)piperidin-1-yl)-3-(4-(1-methylpiperidin-4-yl)phenylamino)pyrazine-2-carboxamide;
(R)-5-(3-(4-fluorobenzamido)piperidin-1-yl)-3-(4-(1-methylpiperidin-4-yl)phenylamino)pyrazine-2-carboxamide;
5-((2R,3R)-3-benzamido-2-methylpiperidin-1-yl)-3-(4-(1-cyclopentylpiperidin-4-yl)phenylamino)pyrazine-2-carboxamide;
5-((2S,3R)-3-benzamido-2-methylpiperidin-1-yl)-3-(4-(1-cyclopentylpiperidin-4-yl)phenylamino)pyrazine-2-carboxamide;
(R)-5-(3-(3-(3-chlorophenyl)-2-oxoimidazolidin-1-yl)piperidin-1-yl)-3-(4-(1-cyclopentylpiperidin-4-yl)phenylamino)pyrazine-2-carboxamide;
(R)-5-(3-benzamidopiperidin-1-yl)-3-(4-(1-cyclopentylpiperidin-4-yl)phenylamino)pyrazine-2-carboxamide;
(R)-3-(4-(1-cyclopentylpiperidin-4-yl)phenylamino)-5-(3-(nicotinamido)piperidin-1-yl)pyrazine-2-carboxamide;
(R)-3-(4-(1-cyclopentylpiperidin-4-yl)phenylamino)-5-(3-(5-fluoronicotinamido)piperidin-1-yl)pyrazine-2-carboxamide;
(R)-3-(4-(1-cyclopentylpiperidin-4-yl)phenylamino)-5-(3-(2-oxopyrrolidin-1-yl)piperidin-1-yl)pyrazine-2-carboxamide;
(R)-3-(4-(1-cyclopentylpiperidin-4-yl)phenylamino)-5-(3-(1-oxoisoindolin-2-yl)piperidin-1-yl)pyrazine-2-carboxamide;
(R)-5-(3-(4-chlorobenzamido)piperidin-1-yl)-3-(4-(1-cyclopropylpiperidin-4-yl)phenylamino)pyrazine-2-carboxamide;
(R)-5-(3-(3-chlorobenzamido)piperidin-1-yl)-3-(4-(1-cyclopropylpiperidin-4-yl)phenylamino)pyrazine-2-carboxamide;
(R)-5-(3-(5-chloronicotinamido)piperidin-1-yl)-3-(4-(1-cyclopropylpiperidin-4-yl)phenylamino)pyrazine-2-carboxamide;
(R)-5-(3-(5-chlorothiophene-2-carboxamido)piperidin-1-yl)-3-(4-(1-cyclopropylpiperidin-4-yl)phenylamino)pyrazine-2-carboxamide;
(R)-5-(3-(benzo[b]thiophene-2-carboxamido)piperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(4,5,6,7-tetrahydrobenzo[b]thiophene-2-carboxamido)piperidin-1-yl)pyrazine-2-carboxamide;
(R)-5-(3-(2-naphthamido)piperidin-1-yl)-3-(4-(1-cyclopropylpiperidin-4-yl)phenylamino)pyrazine-2-carboxamide;
(R)-5-(3-biphenyl-4-ylcarboxamidopiperidin-1-yl)-3-(4-(1-cyclopropylpiperidin-4-yl)phenylamino)pyrazine-2-carboxamide;
(R)-3-(4-(1-cyclopropylpiperidin-4-yl)phenylamino)-5-(3-(6-phenylnicotinamido)piperidin-1-yl)pyrazine-2-carboxamide;
(R)-3-(4-(1-cyclopentylpiperidin-4-yl)phenylamino)-5-(3-(4-fluorobenzamido)piperidin-1-yl)pyrazine-2-carboxamide;
(R)-5-(3-(3-methyl-3-phenylureido)piperidin-1-yl)-3-(phenylamino)pyrazine-2-carboxamide;
(R)-5-(3-(3-(3-chloro-5-(trifluoromethyl)phenyl)-3-methylureido)piperidin-1-yl)-3-(4-fluorophenylamino)pyrazine-2-carboxamide;
(R)-5-(3-(3-(3-chloro-5-(trifluoromethyl)phenyl)-3-methylureido)piperidin-1-yl)-3-(4-(1-cyanocyclopropyl)phenylamino)pyrazine-2-carboxamide;
(R)-3-(4-(1-carbamoylcyclopropyl)phenylamino)-5-(3-(3-(3-chloro-5-(trifluoromethyl)phenyl)-3-methylureido)piperidin-1-yl)pyrazine-2-carboxamide;
(R)—N-(1-(5-carbamoyl-6-(4-(tetrahydro-2H-pyran-4-yl)phenylamino)pyrazin-2-yl)piperidin-3-yl)imidazo[1,2-a]pyridine-6-carboxamide;
(R)—N-(1-(5-carbamoyl-6-(4-(tetrabydro-2H-pyran-4-yl)phenylamino)pyrazin-2-yl)piperidin-3-yl)-5-hydroxyimidazo[1,2-a]pyridine-6-carboxamide;
(R)-3-(cyclopropylamino)-5-(3-(3-methyl-3-phenylureido)piperidin-1-yl)pyrazine-2-carboxamide;
(R)-3-(cyclopentylamino)-5-(3-(3-methyl-3-phenylureido)piperidin-1-yl)pyrazine-2-carboxamide;
(R)-5-(3-(3-(3-chloro-5-(trifluoromethyl)phenyl)-3-methylureido)piperidin-1-yl)-3-(cyclopropylamino)pyrazine-2-carboxamide;
(R)-5-(3-(3-(3-chloro-5-(trifluoromethyl)phenyl)-3-methylureido)piperidin-1-yl)-3-(tetrahydro-2H-pyran-4-ylamino)pyrazine-2-carboxamide;
(R)-5-(3-(3-(tetrabydro-2H-pyran-4-yl)ureido)piperidin-1-yl)-3-(tetrahydro-2H-pyran-4-ylamino)pyrazine-2-carboxamide;
5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide;
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide; or
(R)-3-(5-carbamoyl-6-(3-methylisothiazol-5-ylamino)pyrazin-2-ylamino)-N,N-dimethylazepane-1-carboxamide.

2. The compound of claim 1, wherein the compound has the structure of Formula (A-I):

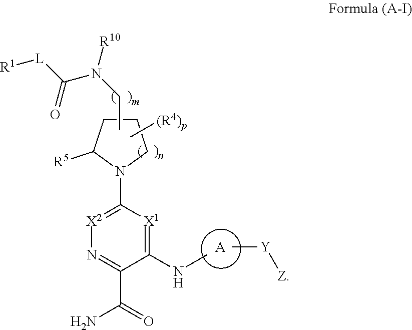

3. The compound of any one of claims 1 or 2, wherein R¹ is substituted or unsubstituted C₆-C₁₂aryl, or substituted or unsubstituted C₁-C₁₂heteroaryl.

4. The compound of any one of claims 1 or 2, wherein R¹ is substituted or unsubstituted C₂-C₄alkenyl, or substituted or unsubstituted C₂-C₄alkynyl.

5. The compound of any one of claims 1-3, wherein R¹ is substituted or unsubstituted isoindolinyl.

6. The compound of any one of claims 1-5, wherein the compound has the structure of Formula (A-IA), (IB), (IC), (ID) or (IE): 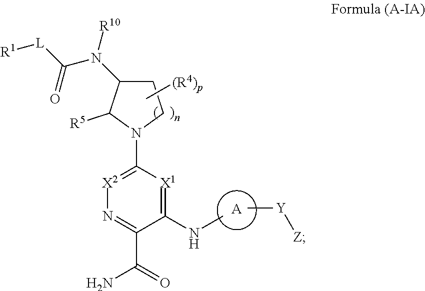 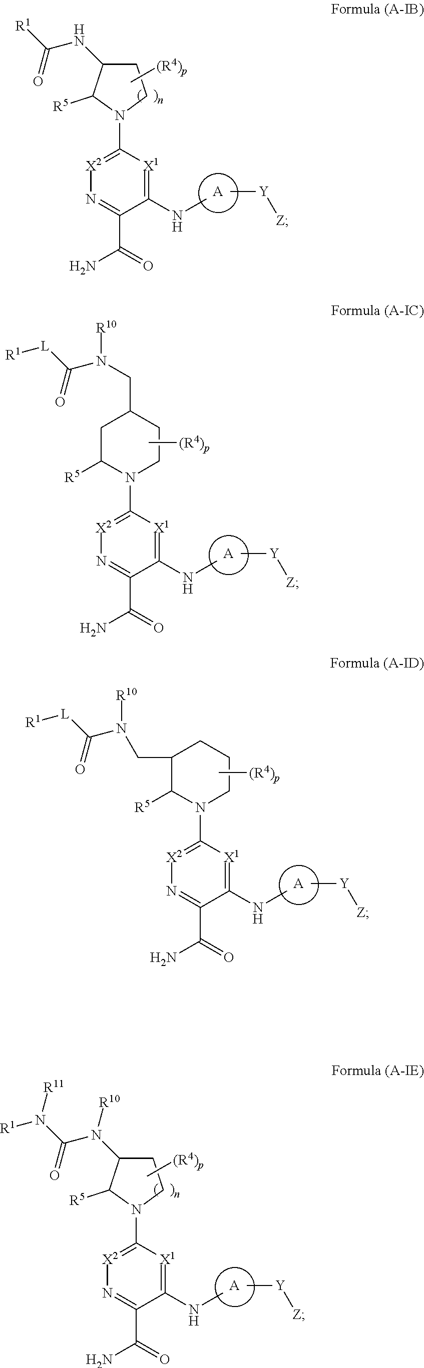
or a pharmaceutically acceptable solvate, pharmaceutically acceptable salt, and/or pharmaceutically acceptable prodrug thereof.

7. The compound of claim 1, wherein the compound has the structure of Formula (A-VI): 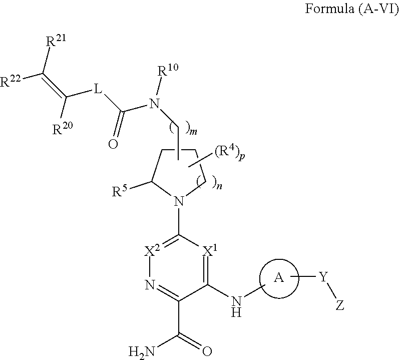
wherein:
R²⁰, R²¹ and R²² are each independently H, CN, halo, substituted or unsubstituted C1-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond;
or a pharmaceutically acceptable solvate, pharmaceutically acceptable salt, and/or pharmaceutically acceptable prodrug thereof.

8. The compound of any one of claims 1-7, wherein R¹⁰ is H or Me.

9. A compound with the structure of Formula (B-I), (B-IA) or (B-IB): 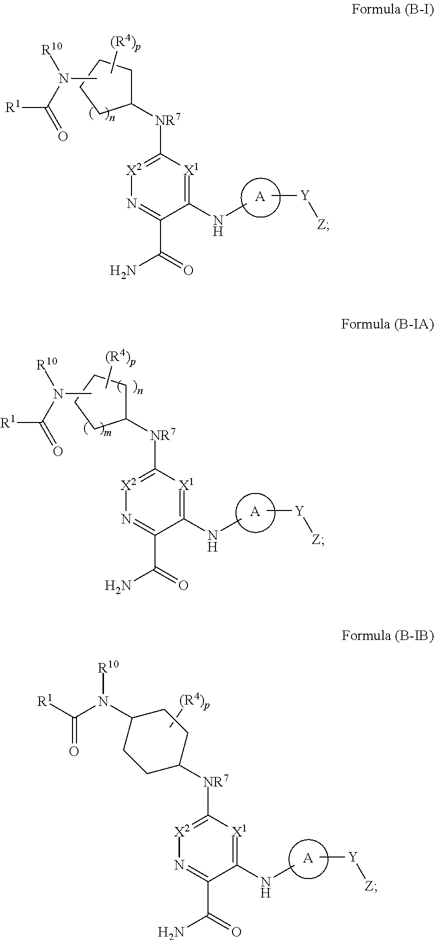
wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is optionally present and when present is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is optionally present and when present is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₅cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is NR⁵R¹¹ or CN; or R¹ and R¹⁰ together with the —C(O)—N— moiety between them form a substituted or unsubstituted C₁-C₁₂heteroaryl or substituted or unsubstituted C₂-C₇heterocycloalkyl optionally fused with a substituted or unsubstituted phenyl ring;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is substituted or unsubstituted C₁-C₆alkyl, substituted or unsubstituted C₂-C₄alkenyl, substituted or unsubstituted C₂-C₄alkynyl, substituted or unsubstituted C₆-C₇cycloalkyl, substituted or unsubstituted C₂-C₇heterocycloalkyl, substituted or unsubstituted C₆-C₁₂aryl, or substituted or unsubstituted C₁-C₁₂heteroaryl;
R⁷ is H, or substituted or unsubstituted C₁-C₄alkyl or C(O)—(C₂-C₄alkenyl);
R¹⁰ and R¹¹ are independently H, or substituted or unsubstituted C₁-C₄alkyl; or R¹⁰ and R¹¹ connect to form a C₁-C₄alkylene;
m is 1, 2, or 3;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when R¹ is substituted or unsubstituted C₂-C₄alkenyl, A is substituted or unsubstituted phenyl, R⁷ is H, the group 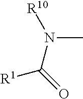
and the group 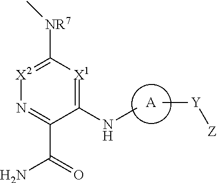
are attached to the same carbon atom or attached to carbon atoms that are adjacent to each other, and X¹ is N, then X² is other than CH or C(Et); and
(2) the compound is other than 5-[[trans-4-(acetylamino)cyclohexyl]amino]-6-ethyl-3-[[3-methyl-4-[4-(4-methyl-1-piperazinyl)-1-piperidinyl]phenyl]amino]-2-pyrazinecarboxamide.

10. The compound according to claim 9, wherein R¹ is substituted or unsubstituted C₆-C₁₂aryl, or substituted or unsubstituted C₁-C₁₂heteroaryl.

11. The compound according to claim 9, wherein R₁ is substituted or unsubstituted C₂-C₄alkenyl, or substituted or unsubstituted C₂-C₄alkynyl.

12. The compound according to any one of claims 9 or 10, wherein R₁ is substituted or unsubstituted isoindolinyl.

13. The compound according to claim 9, wherein the compound is of Formula (B-IIa)-(B-IId) or (B-VIII): 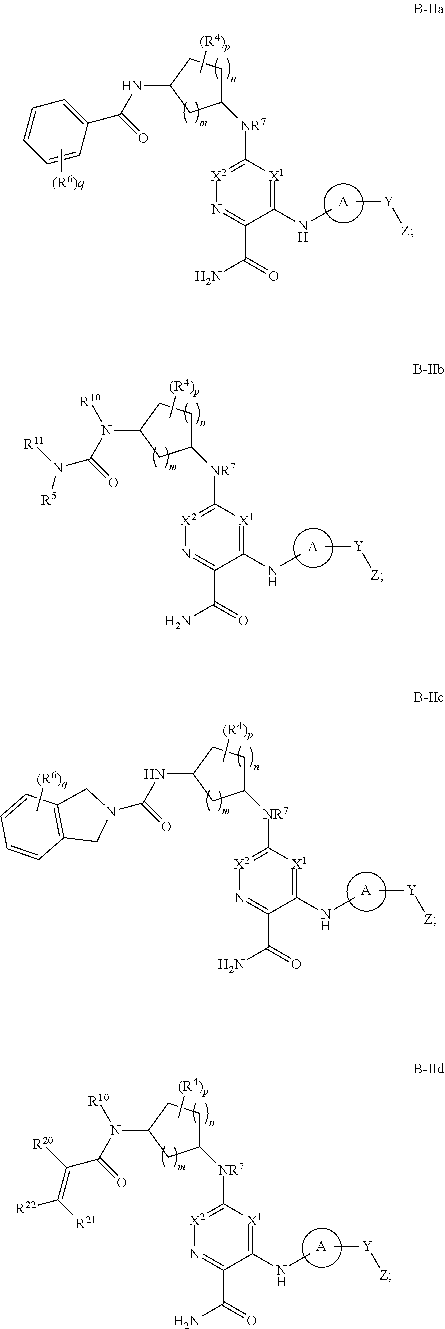 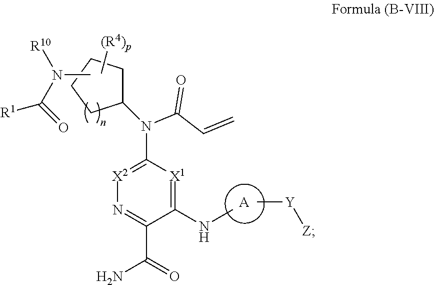
wherein:
each R⁶ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R²⁰, R²¹ and R²² are each independently H, CN, halo, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl; or R²⁰ and R²¹ together form a bond; and
q is 0, 1, 2 or 3;
or a pharmaceutically acceptable solvate, pharmaceutically acceptable salt, or pharmaceutically acceptable prodrug thereof.

14. The compound of any one of claims 9-13, wherein R⁷ is H or Me.

15. A compound of Formula (C-I) having the structure: 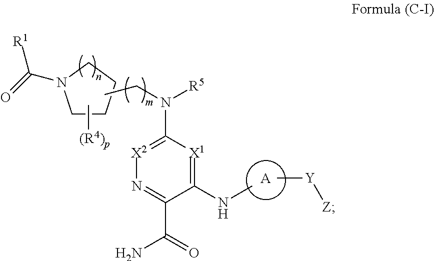
wherein:
ring A is substituted or unsubstituted C₆-C₁₂aryl, or substituted or unsubstituted C₁-C₁₂heteroaryl;
X¹ and X² are both N or are both C(R²); or X¹ is N and X² is C(R²);
Y is a bond, or is —CH₂O—, —OCH₂—, —OCH₂CH₂O—, —O—, —N(R³)—, —C(O)—, —N(R³)C(O)—, —C(O)N(R³)—, —N(R³)C(O)N(R³)—, —S(O)—, —S(O)₂—, —N(R³)S(O)₂—, —S(O)₂N(R³)—, —C(═NH)—, —C(═NH)N(R³)—, —C(═NH)N(R³)—, or substituted or unsubstituted C₁-C₄alkylene;
Z is H, substituted or unsubstituted C₁-C₃alkyl, substituted or unsubstituted C₃-C₆cycloalkyl, substituted or unsubstituted C₂-C₇heterocycloalkyl, substituted or unsubstituted C₆-C₁₂aryl, or substituted or unsubstituted C₁-C₁₂heteroaryl;
R¹ is substituted or unsubstituted C₁-C₄alkyl, substituted or unsubstituted C₂-C₄alkenyl, substituted or unsubstituted C₂-C₄alkynyl, substituted or unsubstituted C₃-C₈cycloalkyl, substituted or unsubstituted C₂-C₇heterocycloalkyl, substituted or unsubstituted C₆-C₁₂aryl, or substituted or unsubstituted C₁-C₁₂heteroaryl; or R¹ is —NR⁷R¹⁰ or CN;
each R² is independently H, —CN, halogen, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
each R³ is independently H, or substituted or unsubstituted C₁-C₄alkyl;
each R⁴ is independently halogen, —CN, —OH, substituted or unsubstituted C₁-C₄alkoxy, substituted or unsubstituted C₁-C₄alkyl, substituted or unsubstituted C₃-C₆cycloalkyl, substituted or unsubstituted C₂-C₆heterocycloalkyl, or —N(R³)₂;
R⁵ is H, or substituted or unsubstituted C₁-C₄alkyl or C(O)—(C₂-C₄alkenyl);
R⁷ is independently substituted or unsubstituted C₁-C₄alkyl, substituted or unsubstituted C₂-C₄alkenyl, substituted or unsubstituted C₂-C₄alkynyl, substituted or unsubstituted C₃-C₆cycloalkyl, substituted or unsubstituted C₂—C-heterocycloalkyl, substituted or unsubstituted C₆-C₁₂aryl, or substituted or unsubstituted C₁-C₁₂heteroaryl:
R¹⁰ is H, or substituted or unsubstituted C₁-C₄alkyl;
m is 0 or 1;
n is 0, 1, 2 or 3; and
p is 0, 1, 2 or 3;
or a pharmaceutically acceptable solvate, pharmaceutically acceptable salt, or pharmaceutically acceptable prodrug thereof;
provided that
(1) when m is 0, then -A-Y—Z is other than 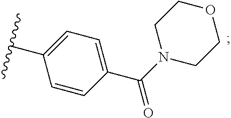 [SMILES: *c1ccc(C(=O)N2CCOCC2)cc1]
(2) when A is quinolinyl, m is 0, X¹ is N and X² is CH, then R¹ is other than Me;
(3) when R¹ is substituted or unsubstituted C₁-C₄alkyl or substituted or unsubstituted C₂-C₄alkenyl, m is 0, and X¹ is N, then X² is CH or N;
(4) the compound is other than
(R)-3-(1-but-2-ynoylpiperidin-3-ylamino)-5-(4-(methylsulfonyl)phenylamino)-1,2,4-triazine-6-carboxamide;
(R,E)-3-(1-(4-(dimethylamino)but-2-enoyl)piperidin-3-ylamino)-5-(4-(methylsulfonyl)phenylamino)-1,2,4-triazine-6-carboxamide;
(R,E)-3-(1-(4-(cyclopropyl(methyl)amino)but-2-enoyl)piperidin-3-ylamino)-5-(4-(methylsulfonyl)phenylamino)-1,2,4-triazine-6-carboxamide;
(R,E)-5-((1-(4-(dimethylamino)but-2-enoyl)piperidin-3-yl)(methyl)amino)-3-(4-phenoxyphenylamino)pyrazine-2-carboxamide; or
(R)-3-(5-carbamoyl-6-(3-methylisothiazol-5-ylamino)pyrazin-2-ylamino)-N,N-dimethylazepane-1-carboxamide;
and a pharmaceutically acceptable solvate, pharmaceutically acceptable salt, or pharmaceutically acceptable prodrug thereof.

16. The compound according to claim 15, wherein R¹ is substituted or unsubstituted C₆-C₁₂aryl, or substituted or unsubstituted C₁-C₁₂heteroaryl.

17. The compound according to claim 15, wherein R¹ is substituted or unsubstituted C₂-C₄alkenyl.

18. The compound according to claim 15, wherein R¹ is substituted or unsubstituted C₂-C₄alkynyl.

19. The compound according to any one of claims 15-18, wherein the compound is of Formula (C-IA), (C-IB) or (C-IC) having the structure: 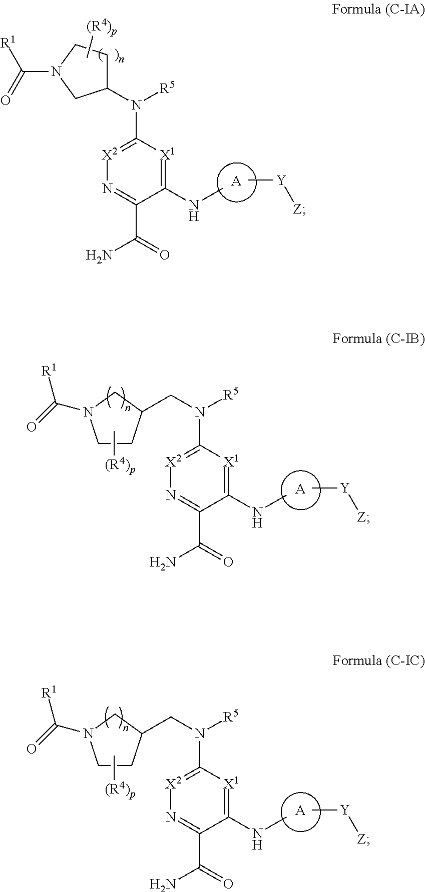
or a pharmaceutically acceptable solvate, pharmaceutically acceptable salt, or pharmaceutically acceptable prodrug thereof,
R²⁰, R²¹ and R²² are each independently H, CN, halo, substituted or unsubstituted C₁-C₄alkyl, substituted or unsubstituted C₃-C₈cycloalkyl, substituted or unsubstituted C₂—C-heterocycloalkyl, substituted or unsubstituted C₆-C₂aryl, or substituted or unsubstituted C₁-C₁₂heteroaryl; or R²⁰ and R²¹ together form a bond.

20. The compound of any one of claims 15-19, wherein R⁵ is H or Me.

21. The compound of any one of claims 1-20, wherein ring A is substituted or unsubstituted C₆-C₁₂aryl.

22. The compound of any one of claims 1-21, wherein ring A is phenyl.

23. The compound of any one of claims 1-20, wherein ring A is substituted or unsubstituted C₁-C₁₂heteroaryl.

24. The compound of claim 23, wherein ring A is pyridyl, pyrazolyl, thiazolyl, isothiazolyl, oxazolyl, or isoxazolyl.

25. The compound of claim 23, wherein ring A is isothiazolyl.

26. The compound of any one of claims 1-25, wherein Y is a bond, —CH₂O—, —OCH₂—, —O—, —N(R³)—, —C(O)—, —N(R³)C(O)—, —C(O)N(R³)—, or substituted or unsubstituted C₁-C₄alkylene.

27. The compound of any one of claims 1-26, wherein Y is a bond, —C(O)—, or —C(O)N(R³)—.

28. The compound of any one of claims 1-27, wherein Z is substituted or unsubstituted C₁-C₃alkyl, substituted or unsubstituted C₂-C₇heterocycloalkyl, substituted or unsubstituted C₆-C₁₂aryl, or substituted or unsubstituted C₁-C₁₂heteroaryl.

29. The compound of claim 28, wherein Z is Me, Et, or i-Pr.

30. The compound of claim 25, wherein ring A is isothiazolyl; Y is a bond; and Z is Me.

31. The compound of any one of claims 1-30, wherein X¹ and X² are both N.

32. The compound of any one of claims 1-30, wherein X¹ and X² are both CH.

33. The compound of any one of claims 1-30, wherein X¹ is N and X² is CH.

34. The compound of any one of claims 1-33, wherein n is 0, 1 or 2.

35. The compound of any one of claims 1-34, wherein n is 2.

36. The compound of any one of claims 1-35, wherein p is 0, 1 or 2.

37. The compound of any one of claims 1-36, wherein p is 0.

38. The compound of claim 13, wherein q is 0, 1 or 2.

39. The compound of claim 38, wherein q is 1.

40. The compound of claim 13, wherein R⁶ is substituted or unsubstituted C₁-C₃alkyl, substituted or unsubstituted C₃-C₆cycloalkyl, or —N(R³)₂.

41. The compound of claim 40, wherein R⁶ is Me, Et, i-Pr, cyclopropyl, cyclobutyl, cyclopentyl, Cl, F, amino, or dimethylamino.

42. The compound of any one of the preceding claims, wherein the compound is selected from the compounds listed in Table N1-N8 or Table 1-Table 17.

43. The compound of claim 1, wherein the compound is any one of compounds selected from the compounds with Compound ID A-1 to A-233, B-1 to B-31, C-1 to C-89, D-1 to D-321, and E-1 to E-158.

44. A pharmaceutical composition comprising a therapeutically effective amount of the compound of any one of claims 1-43, and a pharmaceutically acceptable excipient.

45. The pharmaceutical composition of claim 44, wherein the composition is formulated for a route of administration selected from oral administration, parenteral administration, buccal administration, nasal administration, topical administration, or rectal administration.

46. A method for treating an autoimmune disease or condition comprising administering to a patient in need a therapeutically effective amount of a compound or composition of any one of claims 1-45.

47. The method of claim 46, wherein the autoimmune disease is selected from rheumatoid arthritis or lupus.

48. A method for treating a heteroimmune disease or condition comprising administering to a patient in need a therapeutically effective amount of a compound of any one of claims 1-43.

49. A method for treating a cancer comprising administering to a patient in need a therapeutically effective amount of a compound or composition of any one of claims 1-45.

50. The method of claim 49, wherein the cancer is a B-cell proliferative disorder.

51. The method of claim 49, wherein the B-cell proliferative disorder is diffuse large B cell lymphoma, follicular lymphoma, mantle cell lymphoma, or chronic lymphocytic leukemia.

52. A method for treating mastocytosis comprising administering to a patient in need a therapeutically effective amount of a compound or composition of any one of claims 1-45.

53. A method for treating osteoporosis or bone resorption disorders comprising administering to a patient in need a therapeutically effective amount of a compound or composition of any one of claims 1-45.

54. A method for treating an inflammatory disease or condition comprising administering to a patient in need a therapeutically effective amount of a compound or composition of any one of claims 1-45.
